(19) United States
(12) Patent Application Publication
(10) Pub. No.: US 2025/0092065 A1
(43) Pub. Date: Mar. 20, 2025

(54) 2-(INDAZOL-5-YL)-6-(PIPERIDIN-4-YL)-1,7-NAPHTHYRIDINE DERIVATIVES AND RELATED COMPOUNDS AS MODULATORS FOR SPLICING NUCLEIC ACIDS AND FOR THE TREATMENT OF PROLIFERATIVE DISEASES

(71) Applicant: REMIX THERAPEUTICS INC., Watertown, MA (US)

(72) Inventors: Dominic Reynolds, Stoneham, MA (US); Michael W. Seiler, Belmont, MA (US); Anant A. Agrawal, Waltham, MA (US); Frederic Vaillancourt, Newton, MA (US); Peter Smith, Arlington, MA (US); Sudeep Prajapati, Somerville, MA (US); Allen T. Hopper, Lexington, MA (US); Stepan Vyskocil, Benesov (CZ); Benoît Moreau, Newton, MA (US)

(21) Appl. No.: 18/726,007

(22) PCT Filed: Jan. 5, 2023
(86) PCT No.: PCT/US2023/010228

§ 371 Date: Jul. 1, 2024

Related U.S. Application Data
(60) Provisional application No. 63/296,811, filed on Jan. 5, 2022.

(51) Int. Cl. C07D 519/00; A61K 31/4375; A61K 31/438; A61K 31/4545; A61K 31/496; A61K 31/4985; A61K 31/517; A61K 31/519; A61P 35/00; A61P 35/02; C07D 401/14; C07D 403/14; C07D 417/14; C07D 471/04

(52) U.S. Cl. CPC C07D 519/00; A61K 31/4375; A61K 31/438; A61K 31/4545; A61K 31/496; A61K 31/4985; A61K 31/517; A61K 31/519; A61P 35/00; A61P 35/02; C07D 401/14; C07D 403/14; C07D 417/14; C07D 471/04

(57) ABSTRACT

The present disclosure features compounds and related compositions that, inter alia, modulate nucleic acid splicing, e.g., splicing of a pre-mRNA, as well as methods of use thereof.

### CLAIM OF PRIORITY

This application claims priority to U.S. Application No. 63/296,811, filed on Jan. 5, 2022, the contents of which is incorporated herein by reference in its entirety.

### BACKGROUND

Alternative splicing is a major source of protein diversity in higher eukaryotes, and is frequently regulated in a tissue-specific or development stage-specific manner. Disease associated alternative splicing patterns in pre-mRNAs are often mapped to changes in splice site signals or sequence motifs and regulatory splicing factors (Faustino and Cooper (2003), Genes Dev 17(4):419-37). Current therapies to modulate RNA expression involve oligonucleotide targeting and gene therapy; however, each of these modalities exhibit unique challenges as currently presented. As such, there is a need for new technologies to modulate RNA expression, including the development of small molecule compounds that target splicing.

### SUMMARY

The present disclosure features compounds and related compositions that, inter alia, modulate nucleic acid splicing, e.g., splicing of a pre-mRNA, as well as methods of use thereof. In an embodiment, the compounds described herein are compounds of Formula (I) or (II) and pharmaceutically acceptable salts, solvates, hydrates, tautomers, or stereoisomers thereof. The present disclosure additionally provides methods of using the compounds of the invention (e.g., compounds of Formula (I) or (II) and pharmaceutically acceptable salts, solvates, hydrates, tautomers, stereoisomers thereof), and compositions thereof, e.g., to target, and in embodiments bind or form a complex with, a nucleic acid (e.g., a pre-mRNA or nucleic acid component of a small nuclear ribonucleoprotein (snRNP) or spliceosome), a protein (e.g., a protein component of an snRNP or spliceosome, e.g., a member of the splicing machinery, e.g., one or more of the U1, U2, U4, U5, U6, U11, U12, U4atac, U6atac snRNPs), or a combination thereof. In another aspect, the compounds described herein may be used to alter the composition or structure of a nucleic acid (e.g., a pre-mRNA or mRNA (e.g., a pre-mRNA and the mRNA which arises from the pre-mRNA), e.g., by increasing or decreasing splicing at a splice site. In some embodiments, increasing or decreasing splicing results in modulating the level of a gene product (e.g., an RNA or protein) produced.

In another aspect, the compounds described herein may be used for the prevention and/or treatment of a disease, disorder, or condition, e.g., a disease, disorder or condition associated with splicing, e.g., alternative splicing. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a proliferative disease, disorder, or condition (e.g., a disease, disorder, or condition characterized by unwanted cell proliferation, e.g., a cancer or a benign neoplasm) in a subject. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a non-proliferative disease, disorder, or condition. In some embodiments, the compounds described herein (e.g., compounds of Formula (I) or (II) and pharmaceutically acceptable salts, solvates, hydrates, tautomers, stereoisomers thereof) and compositions thereof are used for the prevention and/or treatment of a neurological disease or disorder, an autoimmune disease or disorder, immunodeficiency disease or disorder, a lysosomal storage disease or disorder, a cardiovascular disease or disorder, a metabolic disease or disorder, a respiratory disease or disorder, a renal disease or disorder, or an infectious disease in a subject.

In one aspect, the present disclosure provides compounds of Formula (I):

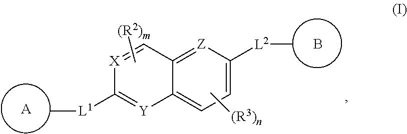

or a pharmaceutically acceptable salt, solvate, hydrate, tautomer, or stereoisomer thereof, wherein each of A, B, L¹, L², X, Y, Z, R², R³, m, n, and subvariables thereof are defined as described herein.

In another aspect, the present disclosure provides compounds of Formula (II):

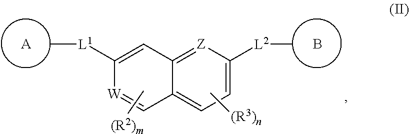

or a pharmaceutically acceptable salt, solvate, hydrate, tautomer, or stereoisomer thereof, wherein each of A, B, Li, L², W, Z, R², R³, m, n, and subvariables thereof are defined as described herein.

In another aspect, the present invention provides pharmaceutical compositions comprising a compound of Formulas (I) or (II), or a pharmaceutically acceptable salt, solvate, hydrate, tautomer, or stereoisomer thereof, and optionally a pharmaceutically acceptable excipient. In an embodiment, the pharmaceutical compositions described herein include an effective amount (e.g., a therapeutically effective amount) of a compound of Formulas (I) or (II), or a pharmaceutically acceptable salt, solvate, hydrate, tautomer, or stereoisomer thereof.

In another aspect, the present disclosure provides methods for modulating splicing, e.g., splicing of a nucleic acid (e.g., a DNA or RNA, e.g., a pre-mRNA) with a compound of Formulas (I) or (II), or a pharmaceutically acceptable salt, solvate, hydrate, tautomer, or stereoisomer thereof. In another aspect, the present disclosure provides compositions for use in modulating splicing, e.g., splicing of a nucleic acid (e.g., a DNA or RNA, e.g., a pre-mRNA) with a compound of Formulas (I) or (II), or a pharmaceutically acceptable salt, solvate, hydrate, tautomer, or stereoisomer thereof. Modulation of splicing may comprise impacting any step involved in splicing and may include an event upstream or downstream of a splicing event. For example, in some embodiments, the compound of Formulas (I) or (II) binds to a target, e.g., a target nucleic acid (e.g., DNA or RNA, e.g., a precursor RNA, e.g., a pre-mRNA), a target protein, or combination thereof (e.g., an snRNP and a pre-mRNA). A target may include a splice site in a pre-mRNA or a component of the splicing machinery, such as the U1 snRNP. In some embodiments, the compound of Formulas (I) or (II) alters a target nucleic acid (e.g., DNA or RNA, e.g., a precursor RNA, e.g., a pre-mRNA), target protein, or combination thereof. In some embodiments, the compound of Formulas (I) or (II) increases or decreases splicing at a splice site on a target nucleic acid (e.g., an RNA, e.g., a precursor RNA, e.g., a pre-mRNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I) or (II), e.g., in a healthy or diseased cell or tissue). In some embodiments, the presence of a compound of Formulas (I) or (II) results an increase or decrease of transcription of a target nucleic acid (e.g., an RNA) by about 0.5% or more (e.g., about 1%, 2%, 3%, 4%, 5%, 10%, 20%, 30%, 40%, 50%, 75%, 90%, 95%, or more), relative to a reference (e.g., the absence of a compound of Formulas (I) or (II), e.g., in a healthy or diseased cell or tissue).

In another aspect, the present disclosure provides methods for preventing and/or treating a disease, disorder, or condition in a subject by administering a compound of Formulas (I) or (II), or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides methods for treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides methods of down-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of up-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides methods of altering the isoform of a target protein with a compound of Formulas (I) or (II), or a pharmaceutically acceptable salt, solvate, hydrate, tautomer, or stereoisomer thereof in a biological sample or subject. Another aspect of the disclosure relates to methods of inhibiting the activity of a target protein in a biological sample or subject. In some embodiments, administration of a compound of Formulas (I) or (II) to a biological sample, a cell, or a subject comprises inhibition of cell growth or induction of cell death.

In another aspect, the present disclosure provides compositions for use in preventing and/or treating a disease, disorder, or condition in a subject by administering a compound of Formulas (I) or (II) or a pharmaceutically acceptable salt, solvate, hydrate, tautomer, or stereoisomer thereof, or related compositions. In some embodiments, the disease or disorder entails unwanted or aberrant splicing. In some embodiments, the disease or disorder is a proliferative disease, disorder, or condition. Exemplary proliferative diseases include cancer, a benign neoplasm, or angiogenesis. In other embodiments, the present disclosure provides methods for treating and/or preventing a non-proliferative disease, disorder, or condition. In still other embodiments, the present disclosure provides compositions for use in treating and/or preventing a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

In another aspect, the present disclosure provides compositions for use in down-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides compositions for use in up-regulating the expression of (e.g., the level of or the rate of production of) a target protein with a compound of Formulas (I) or (II), or a pharmaceutically acceptable salt, solvate, hydrate, tautomer, or stereoisomer thereof in a biological sample or subject. In another aspect, the present disclosure provides compositions for use in altering the isoform of a target protein with a compound of Formulas (I) or (II), or a pharmaceutically acceptable salt, solvate, hydrate, tautomer, or stereoisomer thereof in a biological sample or subject. Another aspect of the disclosure relates to compositions for use in inhibiting the activity of a target protein in a biological sample or subject. In some embodiments, administration of a compound of Formulas (I) or (II) to a biological sample, a cell, or a subject comprises inhibition of cell growth or induction of cell death.

In another aspect, the present disclosure features kits comprising a container with a compound of Formulas (I) or (II), or a pharmaceutically acceptable salt, solvate, hydrate, tautomer, stereoisomer thereof, or a pharmaceutical composition thereof. In certain embodiments, the kits described herein further include instructions for administering the compound of Formulas (I) or (II), or the pharmaceutically acceptable salt, solvate, hydrate, tautomer, stereoisomer thereof, or the pharmaceutical composition thereof.

In any and all aspects of the present disclosure, in some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein other than a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594. In some embodiments, the compound, target nucleic acid (e.g., DNA, RNA, e.g., pre-mRNA), or target protein described herein is a compound, target nucleic acid (e.g., DNA, RNA, e.g., pre-mRNA), or target protein described one of U.S. Pat. No. 8,729,263, U.S. Publication No. 2015/0005289, WO 2014/028459, WO 2016/128343, WO 2016/196386, WO 2017/100726, WO 2018/232039, WO 2018/098446, WO 2019/028440, WO 2019/060917, WO 2019/199972, and WO 2020/004594, each of which is incorporated herein by reference in its entirety.

The details of one or more embodiments of the invention are set forth herein. Other features, objects, and advantages of the invention will be apparent from the Detailed Description, the Examples, and the Claims.

### DETAILED DESCRIPTION

### Selected Chemical Definitions

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorre11, Organic Chemistry, University Science Books, Sausalito, 1999; Smith and March, March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

The abbreviations used herein have their conventional meaning within the chemical and biological arts. The chemical structures and formulae set forth herein are constructed according to the standard rules of chemical valency known in the chemical arts.

When a range of values is listed, it is intended to encompass each value and sub-range within the range. For example “C₁-C₆ alkyl” is intended to encompass, C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆ alkyl.

The following terms are intended to have the meanings presented therewith below and are useful in understanding the description and intended scope of the present invention.

As used herein, “alkyl” refers to a radical of a straight-chain or branched saturated hydrocarbon group having from 1 to 24 carbon atoms (“C₁-C₂₄ alkyl”). In some embodiments, an alkyl group has 1 to 12 carbon atoms (“C₁-C₁₂ alkyl”). In some embodiments, an alkyl group has 1 to 8 carbon atoms (“C₁-C₈ alkyl”). In some embodiments, an alkyl group has 1 to 6 carbon atoms (“C₁-C₆ alkyl”). In some embodiments, an alkyl group has 2 to 6 carbon atoms (“C₂-C₆ alkyl”). In some embodiments, an alkyl group has 1 carbon atom (“C₁ alkyl”). Examples of C₁-C₆alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Each instance of an alkyl group may be independently optionally substituted, i.e., unsubstituted (an “unsubstituted alkyl”) or substituted (a “substituted alkyl”) with one or more substituents; e.g., for instance from 1 to 5 substituents, 1 to 3 substituents, or 1 substituent. In certain embodiments, the alkyl group is unsubstituted C₁-C₁₀ alkyl (e.g., —CH₃). In certain embodiments, the alkyl group is substituted C₁-C₆ alkyl.

As used herein, “alkenyl” refers to a radical of a straight-chain or branched hydrocarbon group having from 2 to 24 carbon atoms, one or more carbon-carbon double bonds, and no triple bonds (“C₂-C₂₄ alkenyl”). In some embodiments, an alkenyl group has 2 to 10 carbon atoms (“C₂-C₁₀ alkenyl”). In some embodiments, an alkenyl group has 2 to 8 carbon atoms (“C₂-C₈ alkenyl”). In some embodiments, an alkenyl group has 2 to 6 carbon atoms (“C₂-C₆ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂-C₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂-C₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Each instance of an alkenyl group may be independently optionally substituted, i.e., unsubstituted (an “unsubstituted alkenyl”) or substituted (a “substituted alkenyl”) with one or more substituents e.g., for instance from 1 to 5 substituents, 1 to 3 substituents, or 1 substituent. In certain embodiments, the alkenyl group is unsubstituted C₁-C₁₀ alkenyl. In certain embodiments, the alkenyl group is substituted C₂-C₆ alkenyl.

As used herein, the term “alkynyl” refers to a radical of a straight-chain or branched hydrocarbon group having from 2 to 24 carbon atoms, one or more carbon-carbon triple bonds (“C₂-C₂₄ alkenyl”). In some embodiments, an alkynyl group has 2 to 10 carbon atoms (“C₂-C₁₀alkynyl”). In some embodiments, an alkynyl group has 2 to 8 carbon atoms (“C₂-C₈ alkynyl”). In some embodiments, an alkynyl group has 2 to 6 carbon atoms (“C₂-C₆ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂-C₄ alkynyl groups include ethynyl (C₂), 1-propynyl (C₃), 2-propynyl (C₃), 1-butynyl (C₄), 2-butynyl (C₄), and the like. Each instance of an alkynyl group may be independently optionally substituted, i.e., unsubstituted (an “unsubstituted alkynyl”) or substituted (a “substituted alkynyl”) with one or more substituents e.g., for instance from 1 to 5 substituents, 1 to 3 substituents, or 1 substituent. In certain embodiments, the alkynyl group is unsubstituted C₂₋₁₀ alkynyl. In certain embodiments, the alkynyl group is substituted C₂₋₆ alkynyl.

As used herein, the term “haloalkyl,” refers to a non-cyclic stable straight or branched chain, or combinations thereof, including at least one carbon atom and at least one halogen selected from the group consisting of F, Cl, Br, and I. The halogen(s) F, Cl, Br, and I may be placed at any position of the haloalkyl group. Exemplary haloalkyl groups include, but are not limited to: —CF₃, —CCl₃, —CH₂—CF₃, —CH₂—CCl₃, —CH₂—CBr₃, —CH₂—Cl₃, —CH₂—CH₂—CH(CF₃)—CH₃, —CH₂—CH₂—CH(Br)—CH₃, and —CH₂—CH═CH—CH₂—CF₃. Each instance of a haloalkyl group may be independently optionally substituted, i.e., unsubstituted (an “unsubstituted haloalkyl”) or substituted (a “substituted haloalkyl”) with one or more substituents e.g., for instance from 1 to 5 substituents, 1 to 3 substituents, or 1 substituent.

As used herein, the term “heteroalkyl,” refers to a non-cyclic stable straight or branched chain, or combinations thereof, including at least one carbon atom and at least one heteroatom selected from the group consisting of O, N, P, Si, and S, and wherein the nitrogen and sulfur atoms may optionally be oxidized, and the nitrogen heteroatom may optionally be quaternized. The heteroatom(s) O, N, P, S, and Si may be placed at any position of the heteroalkyl group. Exemplary heteroalkyl groups include, but are not limited to: —CH₂—CH₂—O—CH₃, —CH₂—CH₂—NH—CH₃, —CH₂—CH₂—N(CH₃)—CH₃, —CH₂—S—CH₂—CH₃, —CH₂—CH₂, —S(O)—CH₃, —CH₂—CH₂—S(O)₂—CH₃, —CH═CHO—CH₃, —Si(CH₃)₃, —CH₂—CH═N—OCH₃, —CH═CH—N(CH₃)—CH₃, —O—CH₃, and —O—CH₂—CH₃. Up to two or three heteroatoms may be consecutive, such as, for example, —CH₂—NH—OCH₃ and —CH₂—O—Si(CH₃)₃. Where “heteroalkyl” is recited, followed by recitations of specific heteroalkyl groups, such as —CH₂O, —NR^{C}R^{D}, or the like, it will be understood that the terms heteroalkyl and —CH₂O or —NR^{C}R^{D} are not redundant or mutually exclusive. Rather, the specific heteroalkyl groups are recited to add clarity. Thus, the term “heteroalkyl” should not be interpreted herein as excluding specific heteroalkyl groups, such as —CH₂O, —NR^{C}R^{D}, or the like. Each instance of a heteroalkyl group may be independently optionally substituted, i.e., unsubstituted (an “unsubstituted heteroalkyl”) or substituted (a “substituted heteroalkyl”) with one or more substituents e.g., for instance from 1 to 5 substituents, 1 to 3 substituents, or 1 substituent.

As used herein, “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆-C₁₄ aryl”). In some embodiments, an aryl group has six ring carbon atoms (“C₆ aryl”; e.g., phenyl). In some embodiments, an aryl group has ten ring carbon atoms (“C₁₀ aryl”; e.g., naphthyl such as 1-naphthyl and 2-naphthyl). In some embodiments, an aryl group has fourteen ring carbon atoms (“C₁₄ aryl”; e.g., anthracyl). An aryl group may be described as, e.g., a C₆-C₁₀-membered aryl, wherein the term “membered” refers to the non-hydrogen ring atoms within the moiety. Aryl groups include phenyl, naphthyl, indenyl, and tetrahydronaphthyl. Each instance of an aryl group may be independently optionally substituted, i.e., unsubstituted (an “unsubstituted aryl”) or substituted (a “substituted aryl”) with one or more substituents. In certain embodiments, the aryl group is unsubstituted C₆-C₁₄ aryl. In certain embodiments, the aryl group is substituted C₆-C₁₄ aryl.

As used herein, “heteroaryl” refers to a radical of a 5-10 membered monocyclic or bicyclic 4n+2 aromatic ring system (e.g., having 6 or 10 π electrons shared in a cyclic array) having ring carbon atoms and 1-4 ring heteroatoms provided in the aromatic ring system, wherein each heteroatom is independently selected from nitrogen, oxygen and sulfur (“5-10 membered heteroaryl”). In heteroaryl groups that contain one or more nitrogen atoms, the point of attachment can be a carbon or nitrogen atom, as valency permits. Heteroaryl bicyclic ring systems can include one or more heteroatoms in one or both rings. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused (aryl/heteroaryl) ring system. Bicyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). A heteroaryl group may be described as, e.g., a 6-10-membered heteroaryl, wherein the term “membered” refers to the non-hydrogen ring atoms within the moiety. Each instance of a heteroaryl group may be independently optionally substituted, i.e., unsubstituted (an “unsubstituted heteroaryl”) or substituted (a “substituted heteroaryl”) with one or more substituents e.g., for instance from 1 to 5 substituents, 1 to 3 substituents, or 1 substituent.

Exemplary 5-membered heteroaryl groups containing one heteroatom include, without limitation, pyrrolyl, furanyl and thiophenyl. Exemplary 5-membered heteroaryl groups containing two heteroatoms include, without limitation, imidazolyl, pyrazolyl, oxazolyl, isoxazolyl, thiazolyl, and isothiazolyl. Exemplary 5-membered heteroaryl groups containing three heteroatoms include, without limitation, triazolyl, oxadiazolyl, and thiadiazolyl. Exemplary 5-membered heteroaryl groups containing four heteroatoms include, without limitation, tetrazolyl. Exemplary 6-membered heteroaryl groups containing one heteroatom include, without limitation, pyridinyl. Exemplary 6-membered heteroaryl groups containing two heteroatoms include, without limitation, pyridazinyl, pyrimidinyl, and pyrazinyl. Exemplary 6-membered heteroaryl groups containing three or four heteroatoms include, without limitation, triazinyl and tetrazinyl, respectively. Exemplary 7-membered heteroaryl groups containing one heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Other exemplary heteroaryl groups include heme and heme derivatives.

As used herein, “cycloalkyl” refers to a radical of a non-aromatic cyclic hydrocarbon group having from 3 to 10 ring carbon atoms (“C₃-C₁₀ cycloalkyl”) and zero heteroatoms in the non-aromatic ring system. In some embodiments, a cycloalkyl group has 3 to 8 ring carbon atoms (“C₃-C₈ cycloalkyl”). In some embodiments, a cycloalkyl group has 3 to 6 ring carbon atoms (“C₃-C₆ cycloalkyl”). In some embodiments, a cycloalkyl group has 3 to 6 ring carbon atoms (“C₃-C₆ cycloalkyl”). In some embodiments, a cycloalkyl group has 5 to 10 ring carbon atoms (“C₅-C₁₀ cycloalkyl”). A cycloalkyl group may be described as, e.g., a C₄-C₇-membered cycloalkyl, wherein the term “membered” refers to the non-hydrogen ring atoms within the moiety. Exemplary C₃-C₆ cycloalkyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃-C₈ cycloalkyl groups include, without limitation, the aforementioned C₃-C₆ cycloalkyl groups as well as cycloheptyl (C₇), cycloheptenyl (C₇), cycloheptadienyl (C₇), cycloheptatrienyl (C₇), cyclooctyl (C₈), cyclooctenyl (C₅), cubanyl (C₅), bicyclo[1.1.1]pentanyl (C₅), bicyclo[2.2.2]octanyl (C₅), bicyclo[2.1.1]hexanyl (C₆), bicyclo[3.1.1]heptanyl (C₇), and the like. Exemplary C₃-C₁₀ cycloalkyl groups include, without limitation, the aforementioned C₃-C₈ cycloalkyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the cycloalkyl group is either monocyclic (“monocyclic cycloalkyl”) or contain a fused, bridged or spiro ring system such as a bicyclic system (“bicyclic cycloalkyl”) and can be saturated or can be partially unsaturated. “Cycloalkyl” also includes ring systems wherein the cycloalkyl ring, as defined above, is fused with one or more aryl groups wherein the point of attachment is on the cycloalkyl ring, and in such instances, the number of carbons continue to designate the number of carbons in the cycloalkyl ring system. Each instance of a cycloalkyl group may be independently optionally substituted, i.e., unsubstituted (an “unsubstituted cycloalkyl”) or substituted (a “substituted cycloalkyl”) with one or more substituents. In certain embodiments, the cycloalkyl group is unsubstituted C₃-C₁₀ cycloalkyl. In certain embodiments, the cycloalkyl group is a substituted C₃-C₁₀ cycloalkyl.

“Heterocyclyl” as used herein refers to a radical of a 3- to 16-membered non-aromatic ring system having ring carbon atoms and 1 to 4 ring heteroatoms, wherein each heteroatom is independently selected from nitrogen, oxygen, sulfur, boron, phosphorus, and silicon (“3-16 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or a fused, bridged or spiro ring system such as a bicyclic system (“bicyclic heterocyclyl”), and can be saturated or can be partially unsaturated. Heterocyclyl bicyclic ring systems can include one or more heteroatoms in one or both rings. “Heterocyclyl” also includes ring systems wherein the heterocyclyl ring, as defined above, is fused with one or more cycloalkyl groups wherein the point of attachment is either on the cycloalkyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. A heterocyclyl group may be described as, e.g., a 3-7-membered heterocyclyl, wherein the term “membered” refers to the non-hydrogen ring atoms, i.e., carbon, nitrogen, oxygen, sulfur, boron, phosphorus, and silicon, within the moiety. Each instance of heterocyclyl may be independently optionally substituted, i.e., unsubstituted (an “unsubstituted heterocyclyl”) or substituted (a “substituted heterocyclyl”) with one or more substituents. In certain embodiments, the heterocyclyl group is unsubstituted 3-16 membered heterocyclyl. In certain embodiments, the heterocyclyl group is substituted 3-16 membered heterocyclyl.

Exemplary 3-membered heterocyclyl groups containing one heteroatom include, without limitation, azirdinyl, oxiranyl, thiorenyl. Exemplary 4-membered heterocyclyl groups containing one heteroatom include, without limitation, azetidinyl, oxetanyl and thietanyl. Exemplary 5-membered heterocyclyl groups containing one heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing two heteroatoms include, without limitation, dioxolanyl, oxasulfuranyl, disulfuranyl, and oxazolidin-2-one. Exemplary 5-membered heterocyclyl groups containing three heteroatoms include, without limitation, triazolinyl, oxadiazolinyl, and thiadiazolinyl. Exemplary 6-membered heterocyclyl groups containing one heteroatom include, without limitation, piperidinyl (e.g., 2,2,6,6-tetramethylpiperidinyl), tetrahydropyranyl, dihydropyridinyl, pyridinonyl (e.g., 1-methylpyridin2-onyl), and thianyl. Exemplary 6-membered heterocyclyl groups containing two heteroatoms include, without limitation, piperazinyl, morpholinyl, pyridazinonyl (2-methylpyridazin-3-onyl), pyrimidinonyl (e.g., 1-methylpyrimidin-2-onyl, 3-methylpyrimidin-4-onyl), dithianyl, dioxanyl. Exemplary 6-membered heterocyclyl groups containing two heteroatoms include, without limitation, triazinanyl. Exemplary 7-membered heterocyclyl groups containing one heteroatom include, without limitation, azepanyl, oxepanyl and thiepanyl. Exemplary 8-membered heterocyclyl groups containing one heteroatom include, without limitation, azocanyl, oxecanyl and thiocanyl. Exemplary 5-membered heterocyclyl groups fused to a C₆ aryl ring (also referred to herein as a 5,6-bicyclic heterocyclyl ring) include, without limitation, indolinyl, isoindolinyl, dihydrobenzofuranyl, dihydrobenzothienyl, benzoxazolinonyl, and the like. Exemplary 5-membered heterocyclyl groups fused to a heterocyclyl ring (also referred to herein as a 5,5-bicyclic heterocyclyl ring) include, without limitation, octahydropyrrolopyrrolyl (e.g., octahydropyrrolo[3,4-c]pyrrolyl), and the like. Exemplary 6-membered heterocyclyl groups fused to a heterocyclyl ring (also referred to as a 4,6-membered heterocyclyl ring) include, without limitation, diazaspirononanyl (e.g., 2,7-diazaspiro[3.5]nonanyl). Exemplary 6-membered heterocyclyl groups fused to an aryl ring (also referred to herein as a 6,6-bicyclic heterocyclyl ring) include, without limitation, tetrahydroquinolinyl, tetrahydroisoquinolinyl, and the like. Exemplary 6-membered heterocyclyl groups fused to a cycloalkyl ring (also referred to herein as a 6,7-bicyclic heterocyclyl ring) include, without limitation, azabicyclooctanyl (e.g., (1,5)-8-azabicyclo[3.2.1]octanyl). Exemplary 6-membered heterocyclyl groups fused to a cycloalkyl ring (also referred to herein as a 6,8-bicyclic heterocyclyl ring) include, without limitation, azabicyclononanyl (e.g., 9-azabicyclo[3.3.1]nonanyl).

The terms “alkylene,” “alkenylene,” “alkynylene,” “haloalkylene,” “heteroalkylene,” “cycloalkylene,” or “heterocyclylene,” alone or as part of another substituent, mean, unless otherwise stated, a divalent radical derived from an alkyl, alkenyl, alkynyl, haloalkylene, heteroalkylene, cycloalkyl, or heterocyclyl respectively. For example, the term “alkenylene,” by itself or as part of another substituent, means, unless otherwise stated, a divalent radical derived from an alkene. An alkylene, alkenylene, alkynylene, haloalkylene, heteroalkylene, cycloalkylene, or heterocyclylene group may be described as, e.g., a C₁-C₆-membered alkylene, C₂-C₆-membered alkenylene, C₂-C₆-membered alkynylene, C₁-C₆-membered haloalkylene, C₁-C₆-membered heteroalkylene, C₃-C₈-membered cycloalkylene, or C₃-C₈-membered heterocyclylene, wherein the term “membered” refers to the non-hydrogen atoms within the moiety. In the case of heteroalkylene and heterocyclylene groups, heteroatoms can also occupy either or both of the chain termini (e.g., alkyleneoxy, alkylenedioxy, alkyleneamino, alkylenediamino, and the like). Still further, no orientation of the linking group is implied by the direction in which the formula of the linking group is written. For example, the formula —C(O)₂R′— may represent both —C(O)₂R′— and —R′C(O)₂—.

As used herein, the terms “cyano” or “—CN” refer to a substituent having a carbon atom joined to a nitrogen atom by a triple bond, e.g., C≡N.

As used herein, the terms “halogen” or “halo” refer to fluorine, chlorine, bromine or iodine.

As used herein, the term “hydroxy” refers to —OH.

As used herein, the term “nitro” refers to a substitutent having two oxygen atoms bound to a nitrogen atom, e.g., —NO₂.

As used herein, the term “nucleobase” as used herein, is a nitrogen-containing biological compounds found linked to a sugar within a nucleoside—the basic building blocks of deoxyribonucleic acid (DNA) and ribonucleic acid (RNA). The primary, or naturally occurring, nucleobases are cytosine (DNA and RNA), guanine (DNA and RNA), adenine (DNA and RNA), thymine (DNA) and uracil (RNA), abbreviated as C, G, A, T, and U, respectively. Because A, G, C, and T appear in the DNA, these molecules are called DNA-bases; A, G, C, and U are called RNA-bases. Adenine and guanine belong to the double-ringed class of molecules called purines (abbreviated as R). Cytosine, thymine, and uracil are all pyrimidines. Other nucleobases that do not function as normal parts of the genetic code, are termed non-naturally occurring. In an embodiment, a nucleobase may be chemically modified, for example, with an alkyl (e.g., methyl), halo, —O-alkyl, or other modification.

As used herein, the term “nucleic acid” refers to deoxyribonucleic acids (DNA) or ribonucleic acids (RNA) and polymers thereof in either single- or double-stranded form. The term “nucleic acid” includes a gene, cDNA, pre-mRNA, or an mRNA. In one embodiment, the nucleic acid molecule is synthetic (e.g., chemically synthesized) or recombinant. Unless specifically limited, the term encompasses nucleic acids containing analogues or derivatives of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions), alleles, orthologs, SNPs, and complementarity sequences as well as the sequence explicitly indicated.

As used herein, “oxo” refers to a carbonyl, i.e., —C(O)—.

The symbol “ [symbol] ” as used herein in relation to a compound of Formula (I) or (II) refers to an attachment point to another moiety or functional group within the compound.

Alkyl, alkenyl, alkynyl, haloalkyl, heteroalkyl, cycloalkyl, heterocyclyl, aryl, and heteroaryl groups, as defined herein, are optionally substituted. In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, such as any of the substituents described herein that result in the formation of a stable compound. The present disclosure contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Two or more substituents may optionally be joined to form aryl, heteroaryl, cycloalkyl, or heterocyclyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

The compounds provided herein may exist in one or more particular geometric, optical, enantiomeric, diasteriomeric, epimeric, stereoisomeric, tautomeric, conformational, or anomeric forms, including but not limited to: cis- and trans-forms; E- and Z-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and half chair-forms; and combinations thereof, hereinafter collectively referred to as “isomers” (or “isomeric forms”).

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. In an embodiment, the stereochemistry depicted in a compound is relative rather than absolute. Isomers can be isolated from mixtures by methods known to those skilled in the art, including chiral high-pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E.L. Eliel, Ed., Univ. of Notre Dame Press, Notre Dame, IN 1972). This disclosure additionally encompasses compounds described herein as individual isomers substantially free of other isomers, and alternatively, as mixtures of various isomers.

As used herein, a pure enantiomeric compound is substantially free from other enantiomers or stereoisomers of the compound (i.e., in enantiomeric excess). In other words, an “S” form of the compound is substantially free from the “R” form of the compound and is, thus, in enantiomeric excess of the “R” form. The term “enantiomerically pure” or “pure enantiomer” denotes that the compound comprises more than 75% by weight, more than 80% by weight, more than 85% by weight, more than 90% by weight, more than 91% by weight, more than 92% by weight, more than 93% by weight, more than 94% by weight, more than 95% by weight, more than 96% by weight, more than 97% by weight, more than 98% by weight, more than 99% by weight, more than 99.5% by weight, or more than 99.9% by weight, of the enantiomer. In certain embodiments, the weights are based upon total weight of all enantiomers or stereoisomers of the compound.

In the compositions provided herein, an enantiomerically pure compound can be present with other active or inactive ingredients. For example, a pharmaceutical composition comprising an enantiomerically pure R-compound can comprise, for example, about 90% excipient and about 10% enantiomerically pure R-compound. In certain embodiments, the enantiomerically pure R-compound in such compositions can, for example, comprise, at least about 95% by weight R-compound and at most about 5% by weight S-compound, by total weight of the compound. For example, a pharmaceutical composition comprising an enantiomerically pure S-compound can comprise, for example, about 90% excipient and about 10% enantiomerically pure S-compound. In certain embodiments, the enantiomerically pure S-compound in such compositions can, for example, comprise, at least about 95% by weight S-compound and at most about 5% by weight R-compound, by total weight of the compound.

In some embodiments, a diastereomerically pure compound can be present with other active or inactive ingredients. For example, a pharmaceutical composition comprising a diastereometerically pure exo compound can comprise, for example, about 90% excipient and about 10% diastereometerically pure exo compound. In certain embodiments, the diastereometerically pure exo compound in such compositions can, for example, comprise, at least about 95% by weight exo compound and at most about 5% by weight endo compound, by total weight of the compound. For example, a pharmaceutical composition comprising a diastereometerically pure endo compound can comprise, for example, about 90% excipient and about 10% diastereometerically pure endo compound. In certain embodiments, the diastereometerically pure endo compound in such compositions can, for example, comprise, at least about 95% by weight endo compound and at most about 5% by weight exo compound, by total weight of the compound.

In some embodiments, an isomerically pure compound can be present with other active or inactive ingredients. For example, a pharmaceutical composition comprising a isomerically pure exo compound can comprise, for example, about 90% excipient and about 10% isomerically pure exo compound. In certain embodiments, the isomerically pure exo compound in such compositions can, for example, comprise, at least about 95% by weight exo compound and at most about 5% by weight endo compound, by total weight of the compound. For example, a pharmaceutical composition comprising an isomerically pure endo compound can comprise, for example, about 90% excipient and about 10% isomerically pure endo compound. In certain embodiments, the isomerically pure endo compound in such compositions can, for example, comprise, at least about 95% by weight endo compound and at most about 5% by weight exo compound, by total weight of the compound.

In certain embodiments, the active ingredient can be formulated with little or no excipient or carrier.

Compound described herein may also comprise one or more isotopic substitutions. For example, H may be in any isotopic form, including ¹H, ²H (D or deuterium), and ³H (T or tritium); C may be in any isotopic form, including ¹²C, ¹³C, and ¹⁴C; O may be in any isotopic form, including ¹⁶O and ¹⁸O; N may be in any isotopic form, including ¹⁴N and ¹⁵N; F may be in any isotopic form, including ¹⁸F, ¹⁹F, and the like.

The term “pharmaceutically acceptable salt” is meant to include salts of the active compounds that are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of pharmaceutically acceptable base addition salts include sodium, potassium, calcium, ammonium, organic amino, or magnesium salt, or a similar salt. When compounds of the present invention contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, e.g., Berge et al, Journal of Pharmaceutical Science 66: 1-19 (1977)). Certain specific compounds of the present invention contain both basic and acidic functionalities that allow the compounds to be converted into either base or acid addition salts. These salts may be prepared by methods known to those skilled in the art. Other pharmaceutically acceptable carriers known to those of skill in the art are suitable for the present invention.

In addition to salt forms, the present disclosure provides compounds in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent.

The term “solvate” refers to forms of the compound that are associated with a solvent, usually by a solvolysis reaction. This physical association may include hydrogen bonding. Conventional solvents include water, methanol, ethanol, acetic acid, DMSO, THF, diethyl ether, and the like. The compounds of Formulas (I) or (II) may be prepared, e.g., in crystalline form, and may be solvated. Suitable solvates include pharmaceutically acceptable solvates and further include both stoichiometric solvates and non-stoichiometric solvates. In certain instances, the solvate will be capable of isolation, for example, when one or more solvent molecules are incorporated in the crystal lattice of a crystalline solid. “Solvate” encompasses both solution-phase and isolable solvates. Representative solvates include hydrates, ethanolates, and methanolates.

The term “hydrate” refers to a compound which is associated with water. Typically, the number of the water molecules contained in a hydrate of a compound is in a definite ratio to the number of the compound molecules in the hydrate. Therefore, a hydrate of a compound may be represented, for example, by the general formula R·x H₂O, wherein R is the compound and wherein x is a number greater than 0. A given compound may form more than one type of hydrates, including, e.g., monohydrates (x is 1), lower hydrates (x is a number greater than 0 and smaller than 1, e.g., hemihydrates (R·0.5H₂O)), and polyhydrates (x is a number greater than 1, e.g., dihydrates (R·2H₂O) and hexahydrates (R·6H₂O)).

The term “tautomer” refers to compounds that are interchangeable forms of a particular compound structure, and that vary in the displacement of hydrogen atoms and electrons. Thus, two structures may be in equilibrium through the movement of R electrons and an atom (usually H). For example, enols and ketones are tautomers because they are rapidly interconverted by treatment with either acid or base. Another example of tautomerism is the aci- and nitro-forms of phenylnitromethane that are likewise formed by treatment with acid or base. Tautomeric forms may be relevant to the attainment of the optimal chemical reactivity and biological activity of a compound of interest.

### Other Definitions

The following definitions are more general terms used throughout the present disclosure.

The articles “a” and “an” refer to one or more than one (e.g., to at least one) of the grammatical object of the article. By way of example, “an element” means one element or more than one element. The term “and/or” means either “and” or “or” unless indicated otherwise.

The term “about” is used herein to mean within the typical ranges of tolerances in the art. For example, “about” can be understood as about 2 standard deviations from the mean. In certain embodiments, about means ±10%. In certain embodiments, about means ±5%. When about is present before a series of numbers or a range, it is understood that “about” can modify each of the numbers in the series or range.

“Acquire” or “acquiring” as used herein, refer to obtaining possession of a value, e.g., a numerical value, or image, or a physical entity (e.g., a sample), by “directly acquiring” or “indirectly acquiring” the value or physical entity. “Directly acquiring” means performing a process (e.g., performing an analytical method or protocol) to obtain the value or physical entity. “Indirectly acquiring” refers to receiving the value or physical entity from another party or source (e.g., a third-party laboratory that directly acquired the physical entity or value). Directly acquiring a value or physical entity includes performing a process that includes a physical change in a physical substance or the use of a machine or device. Examples of directly acquiring a value include obtaining a sample from a human subject. Directly acquiring a value includes performing a process that uses a machine or device, e.g., mass spectrometer to acquire mass spectrometry data.

The terms “administer,” “administering,” or “administration,” as used herein refers to implanting, absorbing, ingesting, injecting, inhaling, or otherwise introducing an inventive compound, or a pharmaceutical composition thereof.

As used herein, the terms “condition,” “disease,” and “disorder” are used interchangeably.

An “effective amount” of a compound of Formulas (I) or (II) refers to an amount sufficient to elicit the desired biological response, i.e., treating the condition. As will be appreciated by those of ordinary skill in this art, the effective amount of a compound of Formulas (I) or (II) may vary depending on such factors as the desired biological endpoint, the pharmacokinetics of the compound, the condition being treated, the mode of administration, and the age and health of the subject. An effective amount encompasses therapeutic and prophylactic treatment. For example, in treating cancer, an effective amount of an inventive compound may reduce the tumor burden or stop the growth or spread of a tumor.

A “therapeutically effective amount” of a compound of Formulas (I) or (II) is an amount sufficient to provide a therapeutic benefit in the treatment of a condition or to delay or minimize one or more symptoms associated with the condition. In some embodiments, a therapeutically effective amount is an amount sufficient to provide a therapeutic benefit in the treatment of a condition or to minimize one or more symptoms associated with the condition. A therapeutically effective amount of a compound means an amount of therapeutic agent, alone or in combination with other therapies, which provides a therapeutic benefit in the treatment of the condition. The term “therapeutically effective amount” can encompass an amount that improves overall therapy, reduces or avoids symptoms or causes of the condition, or enhances the therapeutic efficacy of another therapeutic agent.

The terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprised therein. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types.

“Prevention,” “prevent,” and “preventing” as used herein refers to a treatment that comprises administering a therapy, e.g., administering a compound described herein (e.g., a compound of Formulas (I) or (II)) prior to the onset of a disease, disorder, or condition in order to preclude the physical manifestation of said disease, disorder, or condition. In some embodiments, “prevention,” “prevent,” and “preventing” require that signs or symptoms of the disease, disorder, or condition have not yet developed or have not yet been observed. In some embodiments, treatment comprises prevention and in other embodiments it does not.

A “subject” to which administration is contemplated includes, but is not limited to, humans (i.e., a male or female of any age group, e.g., a pediatric subject (e.g., infant, child, adolescent) or adult subject (e.g., young adult, middle-aged adult, or senior adult)) and/or other non-human animals, for example, mammals (e.g., primates (e.g., cynomolgus monkeys, rhesus monkeys); commercially relevant mammals such as cattle, pigs, horses, sheep, goats, cats, and/or dogs) and birds (e.g., commercially relevant birds such as chickens, ducks, geese, and/or turkeys). In certain embodiments, the animal is a mammal. The animal may be a male or female and at any stage of development. A non-human animal may be a transgenic animal.

As used herein, the terms “treatment,” “treat,” and “treating” refer to reversing, alleviating, delaying the onset of, or inhibiting the progress of one or more of a symptom, manifestation, or underlying cause of a disease, disorder, or condition (e.g., as described herein), e.g., by administering a therapy, e.g., administering a compound described herein (e.g., a compound of Formulas (I) or (II)). In an embodiment, treating comprises reducing, reversing, alleviating, delaying the onset of, or inhibiting the progress of a symptom of a disease, disorder, or condition. In an embodiment, treating comprises reducing, reversing, alleviating, delaying the onset of, or inhibiting the progress of a manifestation of a disease, disorder, or condition. In an embodiment, treating comprises reducing, reversing, alleviating, reducing, or delaying the onset of, an underlying cause of a disease, disorder, or condition. In some embodiments, “treatment,” “treat,” and “treating” require that signs or symptoms of the disease, disorder, or condition have developed or have been observed. In other embodiments, treatment may be administered in the absence of signs or symptoms of the disease or condition, e.g., in preventive treatment. For example, treatment may be administered to a susceptible individual prior to the onset of symptoms (e.g., in light of a history of symptoms and/or in light of genetic or other susceptibility factors). Treatment may also be continued after symptoms have resolved, for example, to delay or prevent recurrence. Treatment may also be continued after symptoms have resolved, for example, to delay or prevent recurrence. In some embodiments, treatment comprises prevention and in other embodiments it does not.

A “proliferative disease” refers to a disease that occurs due to abnormal extension by the multiplication of cells (Walker, Cambridge Dictionary of Biology; Cambridge University Press: Cambridge, UK, 1990). A proliferative disease may be associated with: 1) the pathological proliferation of normally quiescent cells; 2) the pathological migration of cells from their normal location (e.g., metastasis of neoplastic cells); 3) the pathological expression of proteolytic enzymes such as the matrix metalloproteinases (e.g., collagenases, gelatinases, and elastases); 4) the pathological angiogenesis as in proliferative retinopathy and tumor metastasis; or 5) evasion of host immune surveillance and elimination of neoplastic cells. Exemplary proliferative diseases include cancers (i.e., “malignant neoplasms”), benign neoplasms, and angiogenesis.

A “non-proliferative disease” refers to a disease that does not primarily extend through the abnormal multiplication of cells. A non-proliferative disease may be associated with any cell type or tissue type in a subject. Exemplary non-proliferative diseases include neurological diseases or disorders (e.g., a repeat expansion disease); autoimmune disease or disorders; immunodeficiency diseases or disorders; lysosomal storage diseases or disorders; inflammatory diseases or disorders; cardiovascular conditions, diseases, or disorders; metabolic diseases or disorders; respiratory conditions, diseases, or disorders; renal diseases or disorders; and infectious diseases.

### Compounds

In one aspect, the present disclosure features a compound of Formula (I):

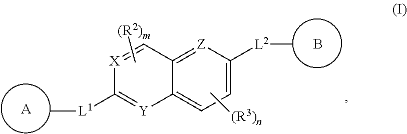

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, cycloalkyl, heterocyclyl, or —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m is 0 or 1; n is 0, 1, or 2; and x is 0, 1, or 2. In an embodiment, when X is CH and Y and Z are each independently N, one of L¹ and L² is independently not N(CH₃) or O. In an embodiment, when X is CH and Y and Z are each independently N, L¹ is not N(CH₃) or O. In an embodiment, when X is CH and Y and Z are each independently N, L² is not N(CH₃) or O. In an embodiment, when X is CH and Y and Z are each independently N, each of L¹ and L² is independently not N(CH₃) or O.

In another aspect, the present invention features a compound of Formula (II):

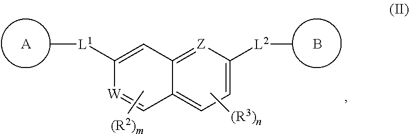

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; W and Z are each N or C(R⁶), wherein at least one of W and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

As generally described herein for compounds of Formula (I) and (II), each of A or B are independently cycloalkyl, heterocyclyl, aryl, or heteroaryl, each of which is optionally substituted with one or more R¹.

In some embodiments, each of A and B are independently a monocyclic ring, e.g., monocyclic cycloalkyl, monocyclic heterocyclyl, monocyclic aryl, or monocyclic heteroaryl. The monocyclic ring may be saturated, partially unsaturated, or fully unsaturated (e.g., aromatic). In some embodiments, A or B are independently a monocyclic ring comprising between 3 and 10 ring atoms (e.g., 3, 4, 5, 6, 7, 8, 9, or 10 ring atoms). In some embodiments, A is a 4-membered monocyclic ring. In some embodiments, B is a 4-membered monocyclic ring. In some embodiments, A is a 5-membered monocyclic ring. In some embodiments, B is a 5-membered monocyclic ring. In some embodiments, A is a 6-membered monocyclic ring. In some embodiments, B is a 6-membered monocyclic ring. In some embodiments, A is a 7-membered monocyclic ring. In some embodiments, B is a 7-membered monocyclic ring. In some embodiments, A is an 8-membered monocyclic ring. In some embodiments, B is an 8-membered monocyclic ring. In some embodiments, A or B are independently a monocyclic ring optionally substituted with one or more R¹.

In some embodiments, A or B are independently a bicyclic ring, e.g., bicyclic cycloalkyl, bicyclic heterocyclyl, bicyclic aryl, or bicyclic heteroaryl. The bicyclic ring may be saturated, partially unsaturated, or fully unsaturated (e.g., aromatic). In some embodiments, A or B are independently a bicyclic ring comprising a fused, bridged, or spiro ring system. In some embodiments, A or B are independently a bicyclic ring comprising between 4 and 18 ring atoms (e.g., 4, 5, 6, 7, 8, 9, 10, 11, 12, 13, 14, 15, 16, 17, or 18 ring atoms). In some embodiments, A is a 6-membered bicyclic ring. In some embodiments, B is a 6-membered bicyclic ring. In some embodiments, A is a 7-membered bicyclic ring. In some embodiments, B is a 7-membered bicyclic ring. In some embodiments, A is an 8-membered bicyclic ring. In some embodiments, B is an 8-membered bicyclic ring. In some embodiments, A is a 9-membered bicyclic ring. In some embodiments, B is a 9-membered bicyclic ring. In some embodiments, A is a 10-membered bicyclic ring. In some embodiments, B is a 10-membered bicyclic ring. In some embodiments, A is an 11-membered bicyclic ring. In some embodiments, B is an 11-membered bicyclic ring. In some embodiments, A is a 12-membered bicyclic ring. In some embodiments, B is a 12-membered bicyclic ring. In some embodiments, A or B are independently a bicyclic ring optionally substituted with one or more R¹.

In some embodiments, A or B are independently a tricyclic ring, e.g., tricyclic cycloalkyl, tricyclic heterocyclyl, tricyclic aryl, or tricyclic heteroaryl. The tricyclic ring may be saturated, partially unsaturated, or fully unsaturated (e.g., aromatic). In some embodiments, A or B are independently a tricyclic ring that comprises a fused, bridged, or spiro ring system, or a combination thereof. In some embodiments, A or B are independently a tricyclic ring comprising between 6 and 24 ring atoms (e.g., 6, 7, 8, 9, 10, 11, 12, 13, 14, 15, 16, 17, 18, 19, 20, 21, 22, 23, or 24 ring atoms). In some embodiments, A is an 8-membered tricyclic ring. In some embodiments, B is an 8-membered tricyclic ring. In some embodiments, A is a 9-membered tricyclic ring. In some embodiments, B is a 9-membered tricyclic ring. In some embodiments, A is a 10-membered tricyclic ring. In some embodiments, B is a 10-membered tricyclic ring. In some embodiments, A or B are independently a tricyclic ring optionally substituted with one or more R¹.

In some embodiments, A or B are independently monocyclic cycloalkyl, monocyclic heterocyclyl, monocyclic aryl, or monocyclic heteroaryl. In some embodiments, A or B are independently bicyclic cycloalkyl, bicyclic heterocyclyl, bicyclic aryl, or bicyclic heteroaryl. In some embodiments, A or B are independently tricyclic cycloalkyl, tricyclic heterocyclyl, tricyclic aryl, or tricyclic heteroaryl. In some embodiments, A is monocyclic heterocyclyl. In some embodiments, B is monocyclic heterocyclyl. In some embodiments, A is bicyclic heterocyclyl. In some embodiments, B is bicyclic heterocyclyl. In some embodiments, A is monocyclic heteroaryl. In some embodiments, B is monocyclic heteroaryl. In some embodiments, A is bicyclic heteroaryl. In some embodiments, B is bicyclic heteroaryl.

In some embodiments, A or B are independently a nitrogen-containing heterocyclyl, e.g., heterocyclyl comprising one or more nitrogen atom. The one or more nitrogen atom of the nitrogen-containing heterocyclyl may be at any position of the ring. In some embodiments, the nitrogen-containing heterocyclyl is monocyclic, bicyclic, or tricyclic. In some embodiments, A or B are independently heterocyclyl comprising at least 1, at least 2, at least 3, at least 4, at least 5, or at least 6 nitrogen atoms. In some embodiments, A is heterocyclyl comprising 1 nitrogen atom. In some embodiments, B is heterocyclyl comprising 1 nitrogen atom. In some embodiments, A is heterocyclyl comprising 2 nitrogen atoms. In some embodiments, B is heterocyclyl comprising 2 nitrogen atoms. In some embodiments, A is heterocyclyl comprising 3 nitrogen atoms. In some embodiments, B is heterocyclyl comprising 3 nitrogen atoms. In some embodiments, A is heterocyclyl comprising 4 nitrogen atoms. In some embodiments, B is heterocyclyl comprising 4 nitrogen atoms. In some embodiments, A or B are independently a nitrogen-containing heterocyclyl comprising one or more additional heteroatoms, e.g., one or more of oxygen, sulfur, boron, silicon, or phosphorus. In some embodiments, the one or more nitrogen of the nitrogen-containing heterocyclyl is substituted, e.g., with R¹.

In some embodiments, A or B are independently a nitrogen-containing heteroaryl, e.g., heteroaryl comprising one or more nitrogen atom. The one or more nitrogen atom of the nitrogen-containing heteroaryl may be at any position of the ring. In some embodiments, the nitrogen-containing heteroaryl is monocyclic, bicyclic, or tricyclic. In some embodiments, A or B are independently heteroaryl comprising at least 1, at least 2, at least 3, at least 4, at least 5, or at least 6 nitrogen atoms. In some embodiments, A is heteroaryl comprising 1 nitrogen atom. In some embodiments, B is heteroaryl comprising 1 nitrogen atom. In some embodiments, A is heteroaryl comprising 2 nitrogen atoms. In some embodiments, B is heteroaryl comprising 2 nitrogen atoms. In some embodiments, A is heteroaryl comprising 3 nitrogen atoms. In some embodiments, B is heteroaryl comprising 3 nitrogen atoms. In some embodiments, A is heteroaryl comprising 4 nitrogen atoms. In some embodiments, B is heteroaryl comprising 4 nitrogen atoms. In some embodiments, A or B are independently a nitrogen-containing heteroaryl comprising one or more additional heteroatoms, e.g., one or more of oxygen, sulfur, boron, silicon, or phosphorus. In some embodiments, the one or more nitrogen of the nitrogen-containing heteroaryl is substituted, e.g., with R¹.

In some embodiments, A is a 6-membered nitrogen-containing heterocyclyl, e.g., a 6-membered heterocyclyl comprising one or more nitrogen. In some embodiments, A is a 6-membered heterocyclyl comprising 1 nitrogen atom. In some embodiments, A is a 6-membered heterocyclyl comprising 2 nitrogen atoms. In some embodiments, A is a 6-membered heterocyclyl comprising 3 nitrogen atoms. In some embodiments, A is a 6-membered heterocyclyl comprising 4 nitrogen atoms. The one or more nitrogen atom of the 6-membered nitrogen-containing heterocyclyl may be at any position of the ring. In some embodiments, A is a 6-membered nitrogen-containing heterocyclyl optionally substituted with one or more R¹. In some embodiments, the one or more nitrogen of the 6-membered nitrogen-containing heterocyclyl is substituted, e.g., with R¹. In some embodiments, A is a 6-membered nitrogen-containing heterocyclyl comprising one or more additional heteroatoms, e.g., one or more of oxygen, sulfur, boron, silicon, or phosphorus.

In some embodiments, B is a 5-membered nitrogen-containing heterocyclyl or heteroaryl, e.g., a 5-membered heterocyclyl or heteroaryl comprising one or more nitrogen. In some embodiments, B is a 5-membered heterocyclyl comprising 1 nitrogen atom. In some embodiments, B is a 5-membered heteroaryl comprising 1 nitrogen atom. In some embodiments, B is a 5-membered heterocyclyl comprising 2 nitrogen atoms. In some embodiments, B is a 5-membered heteroaryl comprising 2 nitrogen atoms. In some embodiments, B is a 5-membered heterocyclyl comprising 3 nitrogen atoms. In some embodiments, B is a 5-membered heteroaryl comprising 3 nitrogen atoms. The one or more nitrogen atom of the 5-membered nitrogen-containing heterocyclyl or heteroaryl may be at any position of the ring. In some embodiments, B is a 5-membered nitrogen-containing heterocyclyl optionally substituted with one or more R¹. In some embodiments, B is a 5-membered nitrogen-containing heteroaryl optionally substituted with one or more R¹. In some embodiments, the one or more nitrogen of the 5-membered nitrogen-containing heterocyclyl or heteroaryl is substituted, e.g., with R¹. In some embodiments, B is a 5-membered nitrogen-containing heterocyclyl or heteroaryl comprising one or more additional heteroatoms, e.g., one or more of oxygen, sulfur, boron, silicon, or phosphorus.

In some embodiments, B is a nitrogen-containing bicyclic heteroaryl (e.g., a 9-membered nitrogen-containing bicyclic heteroaryl), that is optionally substituted with one or more R¹. In some embodiments, B is a 9-membered bicyclic heteroaryl comprising 1 nitrogen atom. In some embodiments, B is a 9-membered bicyclic heteroaryl comprising 2 nitrogen atoms. In some embodiments, B is a 9-membered bicyclic heteroaryl comprising 3 nitrogen atoms. In some embodiments, B is a 9-membered bicyclic heteroaryl comprising 4 nitrogen atoms. The one or more nitrogen atom of the 9-membered bicyclic heteroaryl may be at any position of the ring. In some embodiments, B is a 9-membered bicyclic heteroaryl substituted with one or more R¹.

In some embodiments, each of A and B are independently selected from:

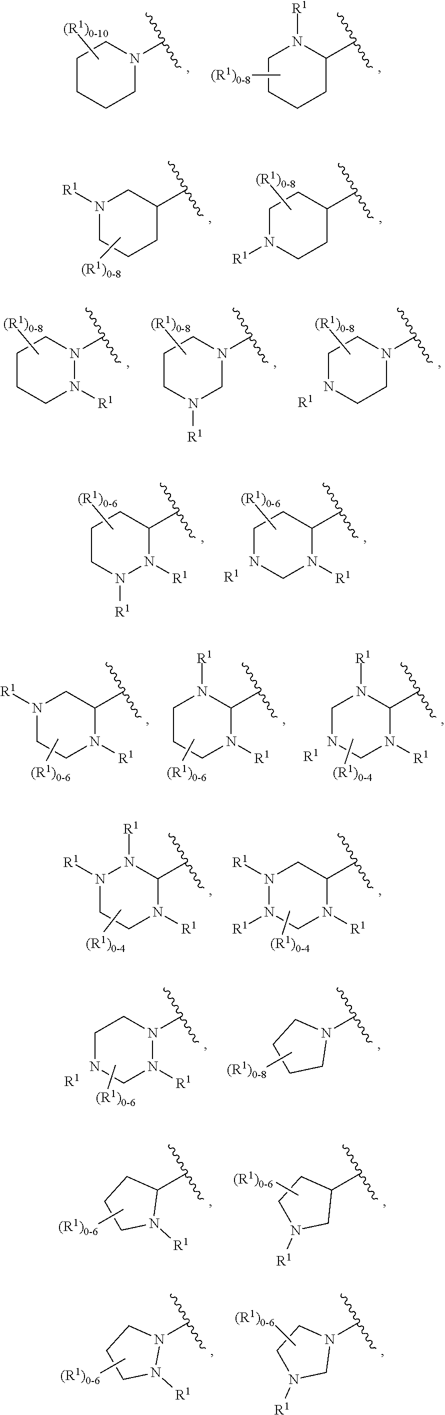

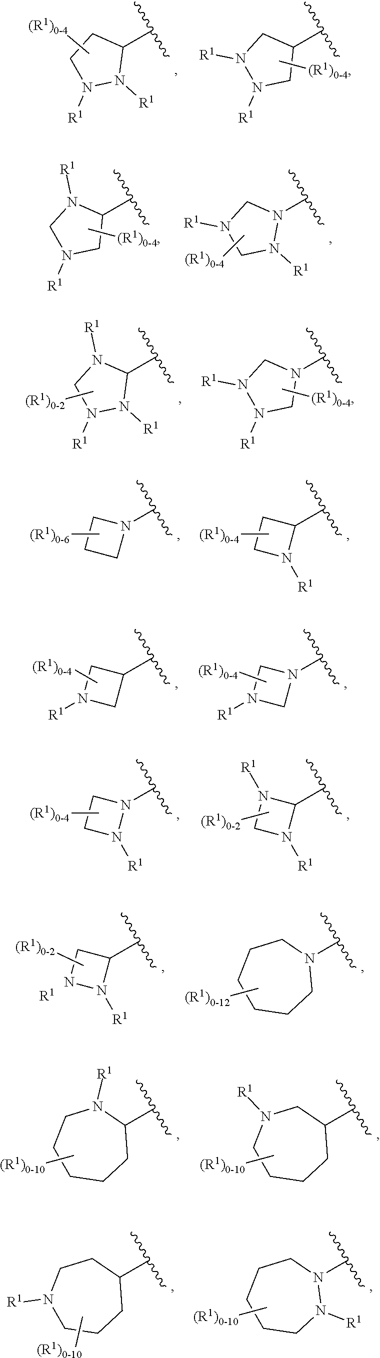

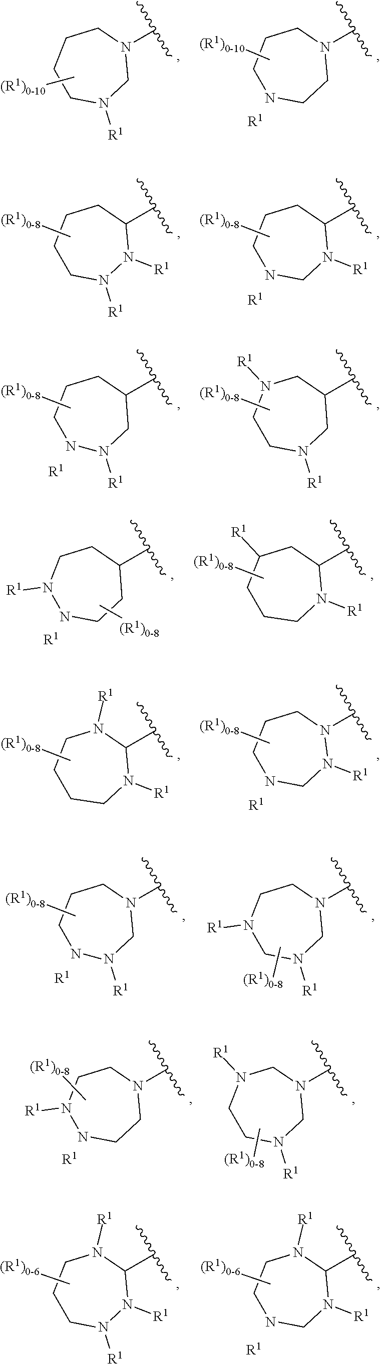

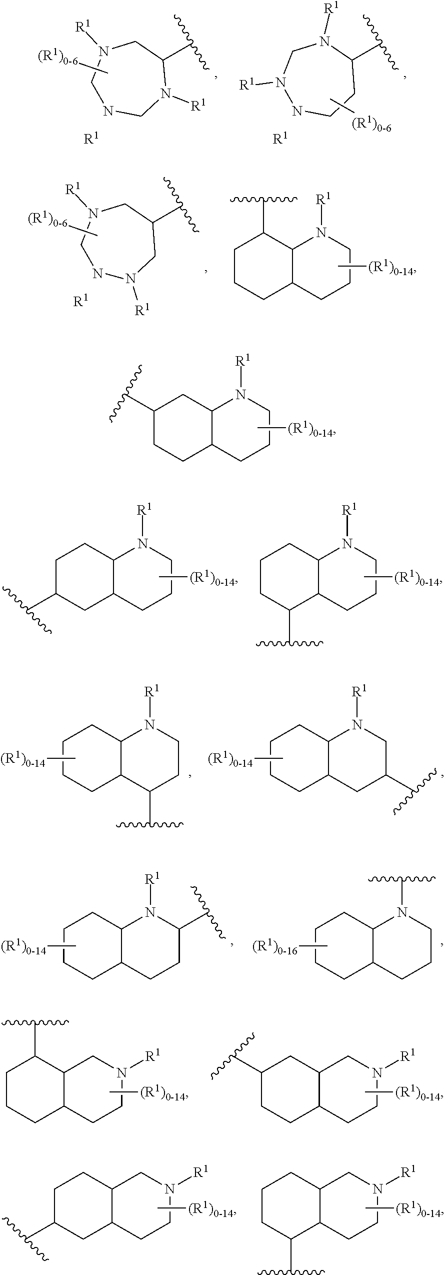

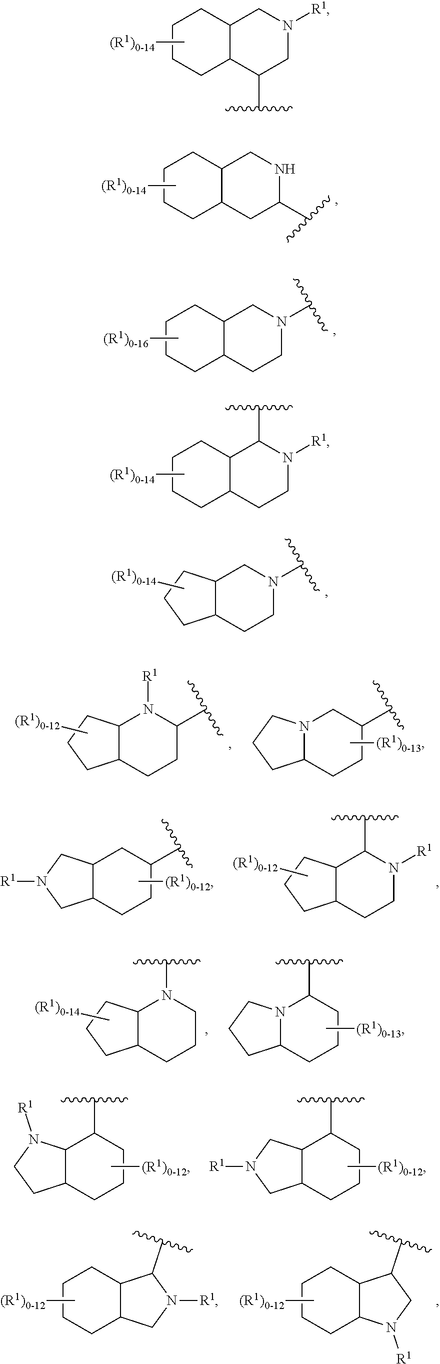

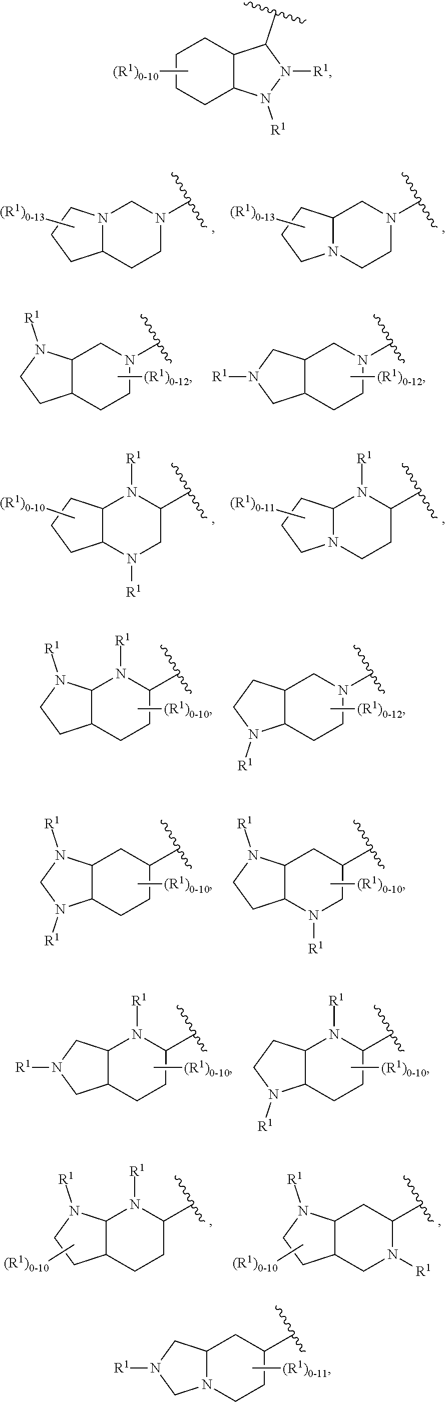

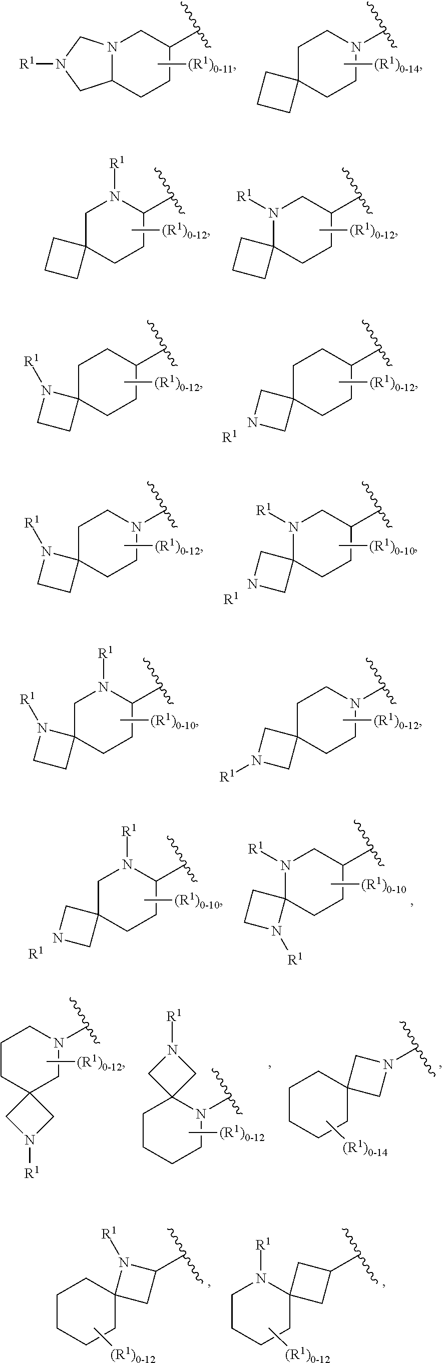

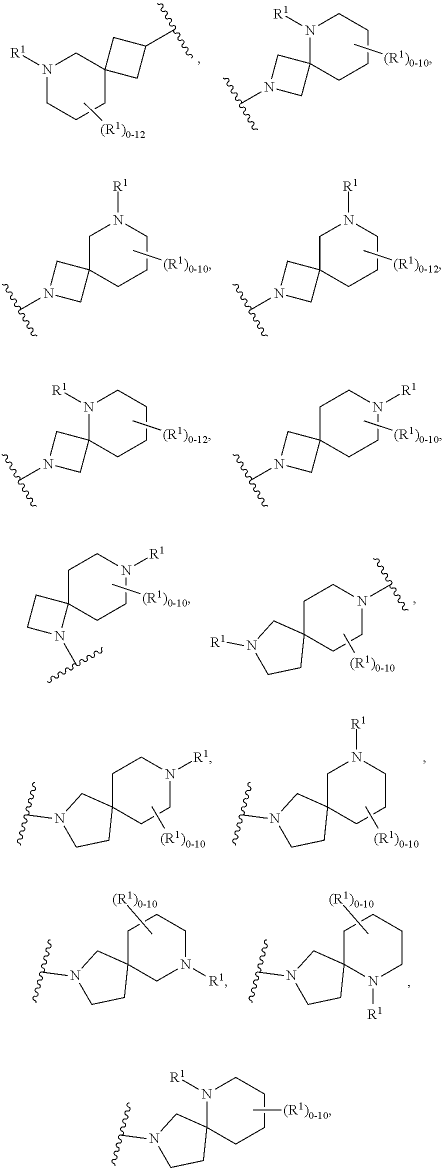

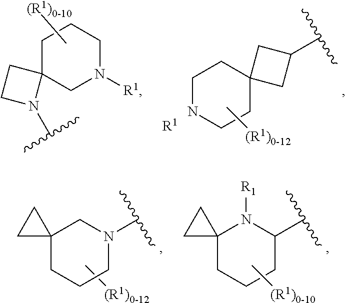

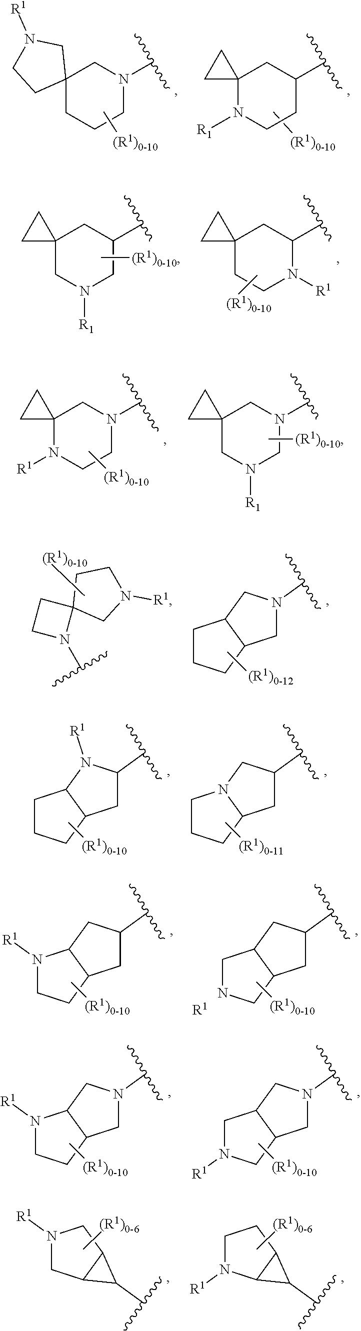

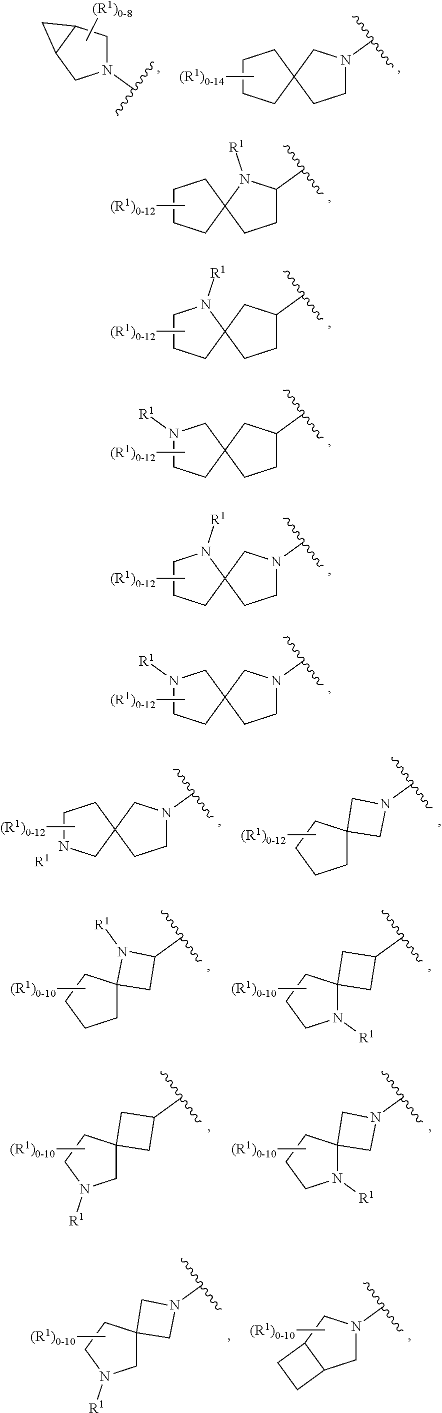

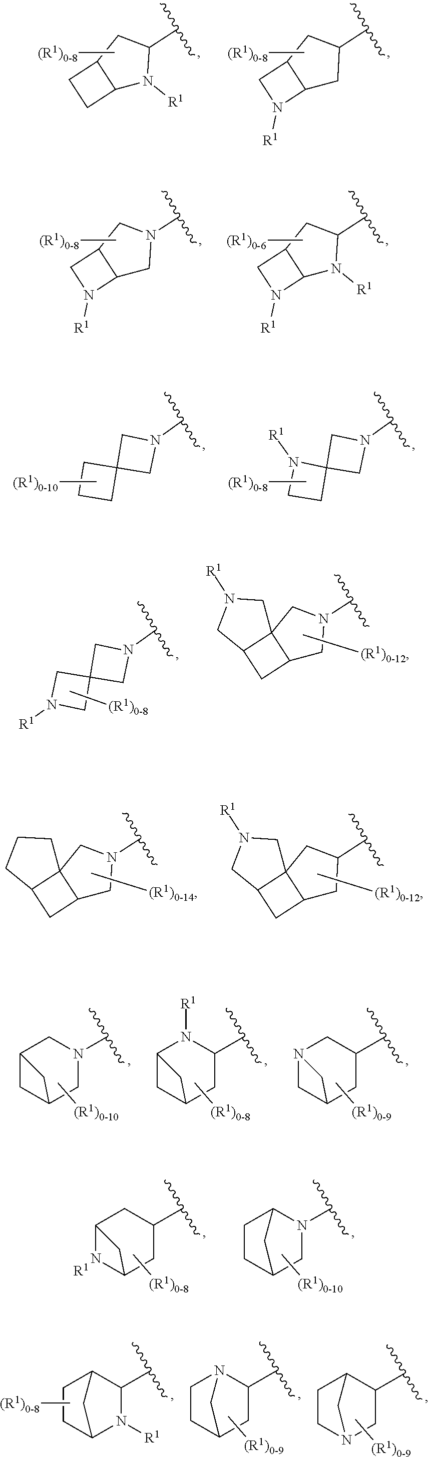

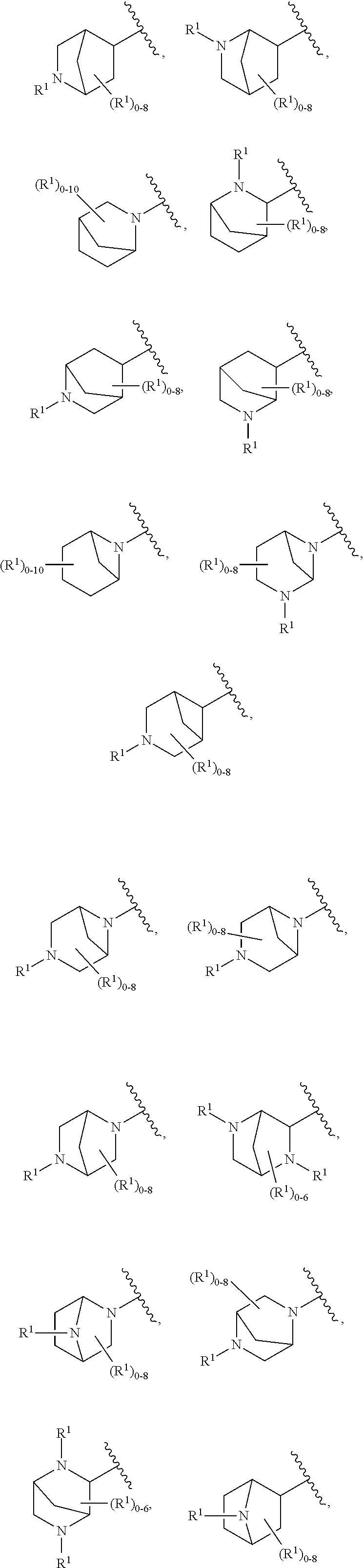

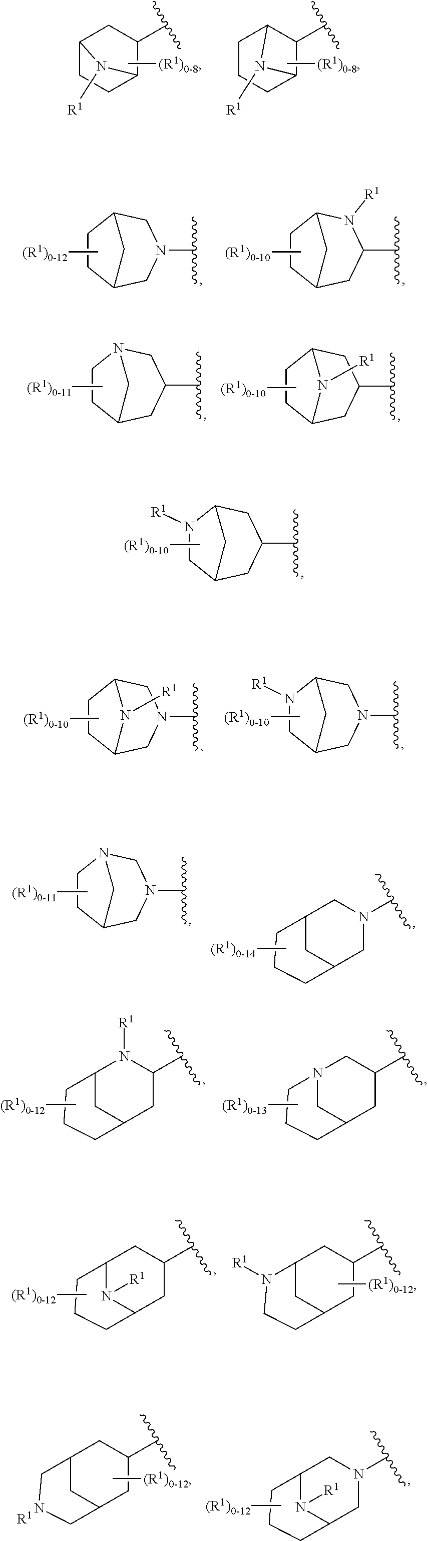

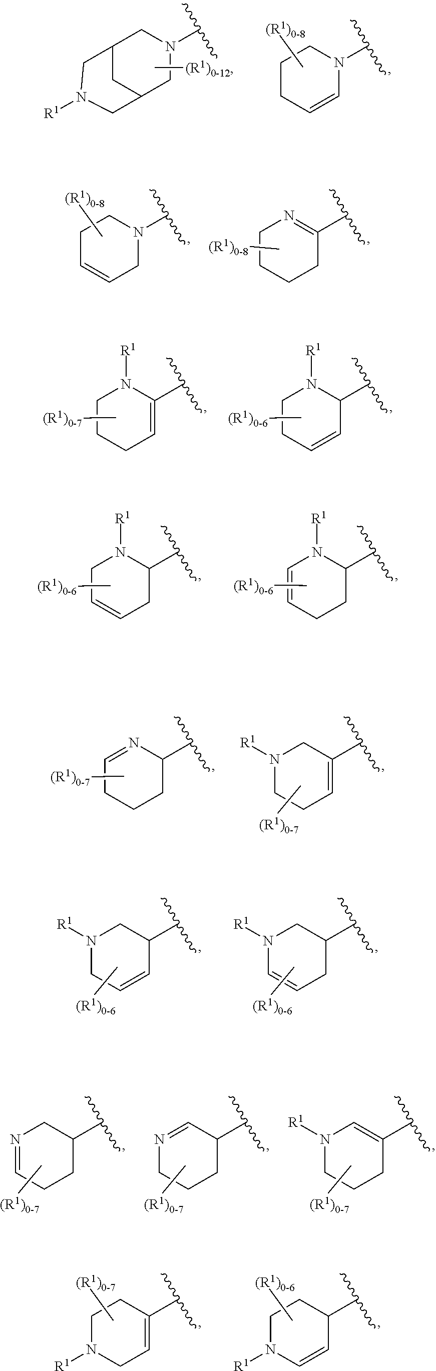

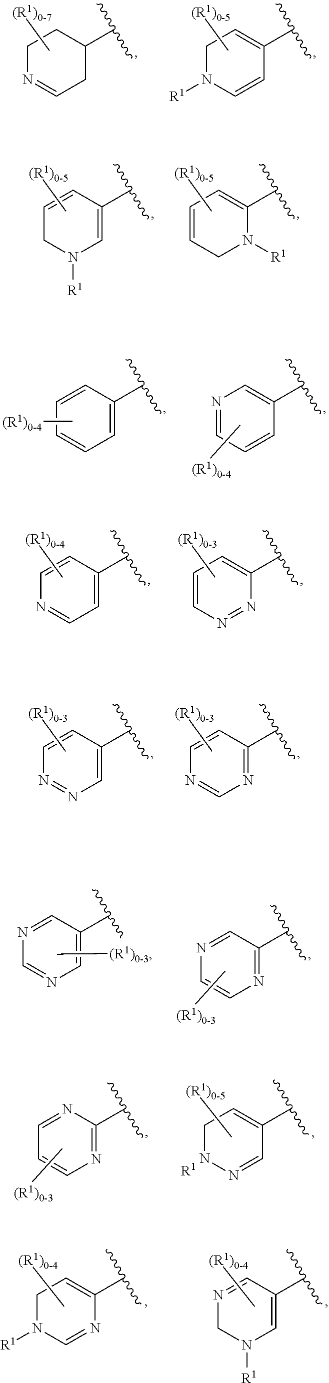

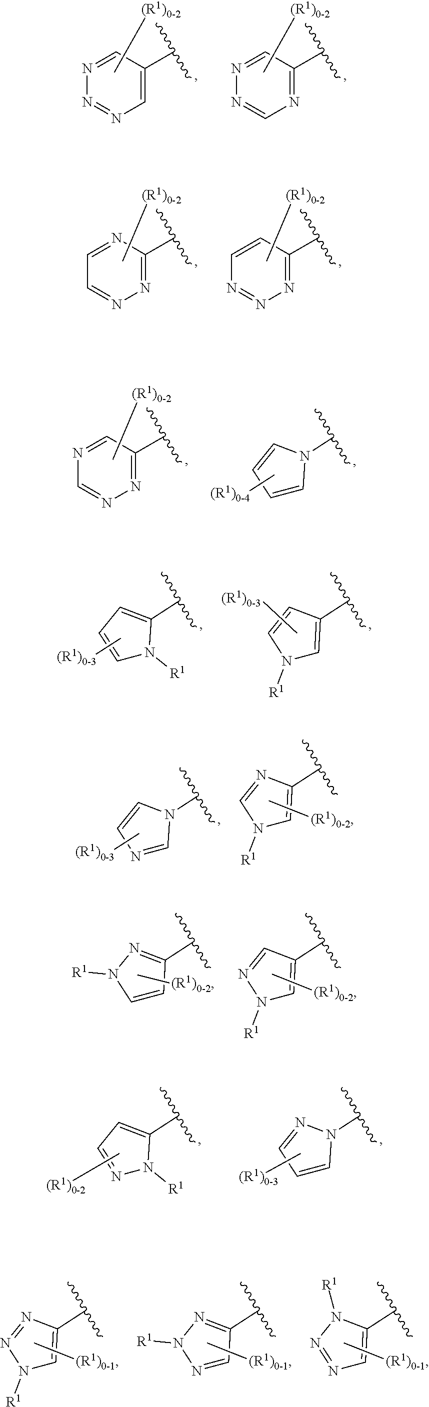

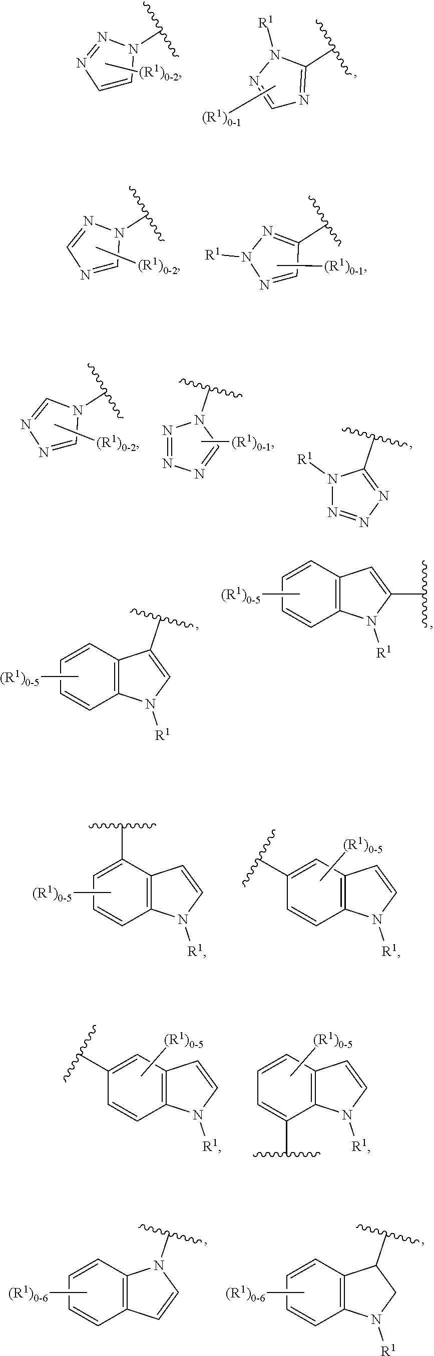

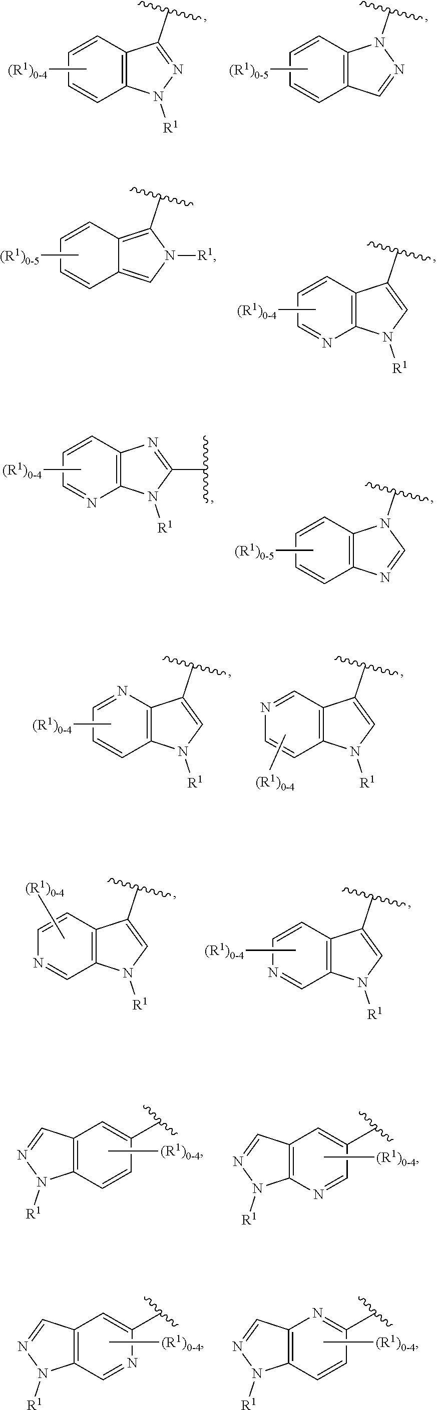

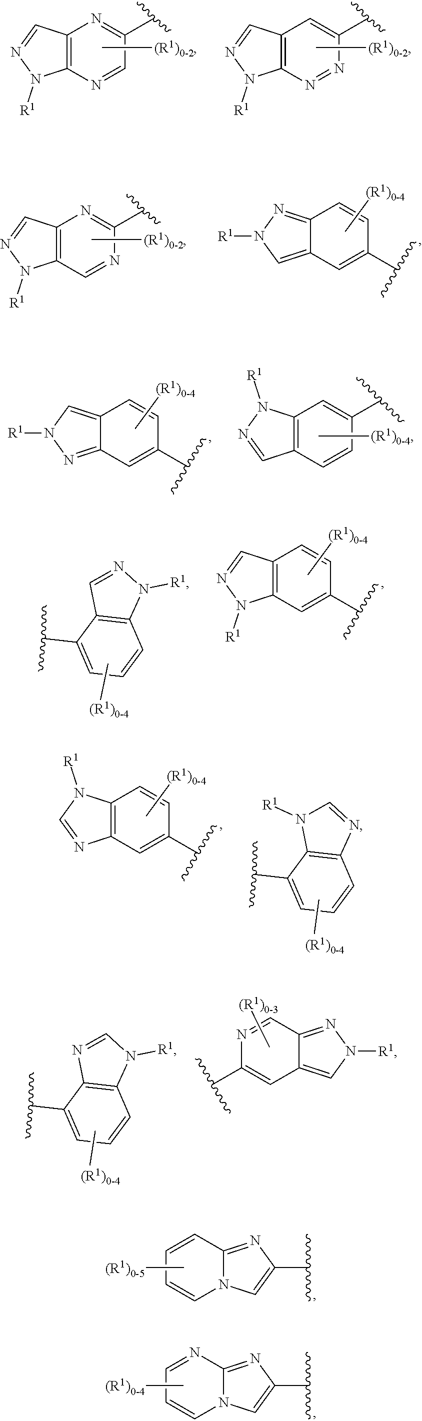

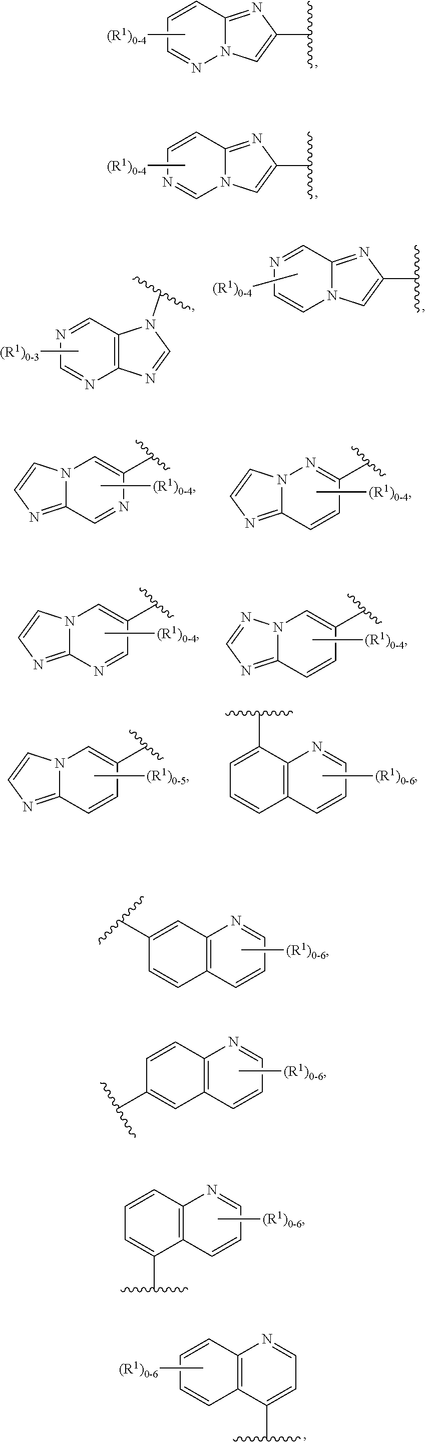

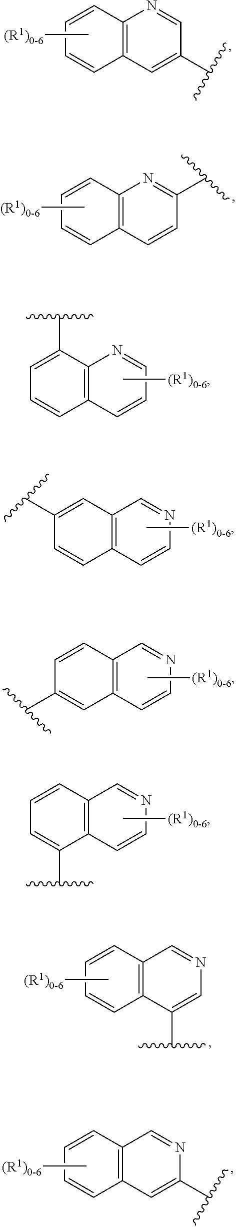

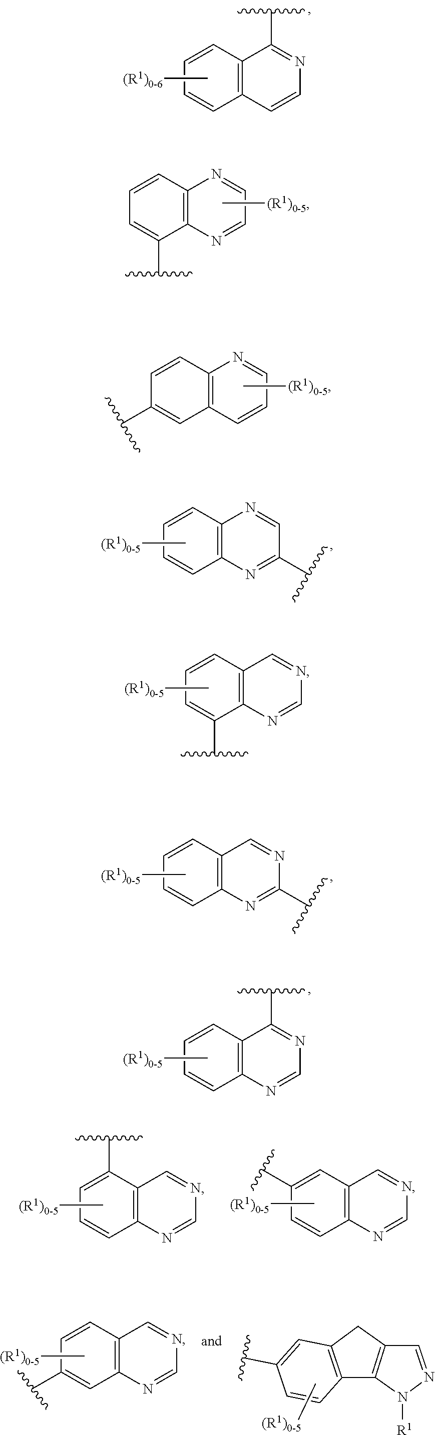

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.

In some embodiments, each of A and B are independently selected from:

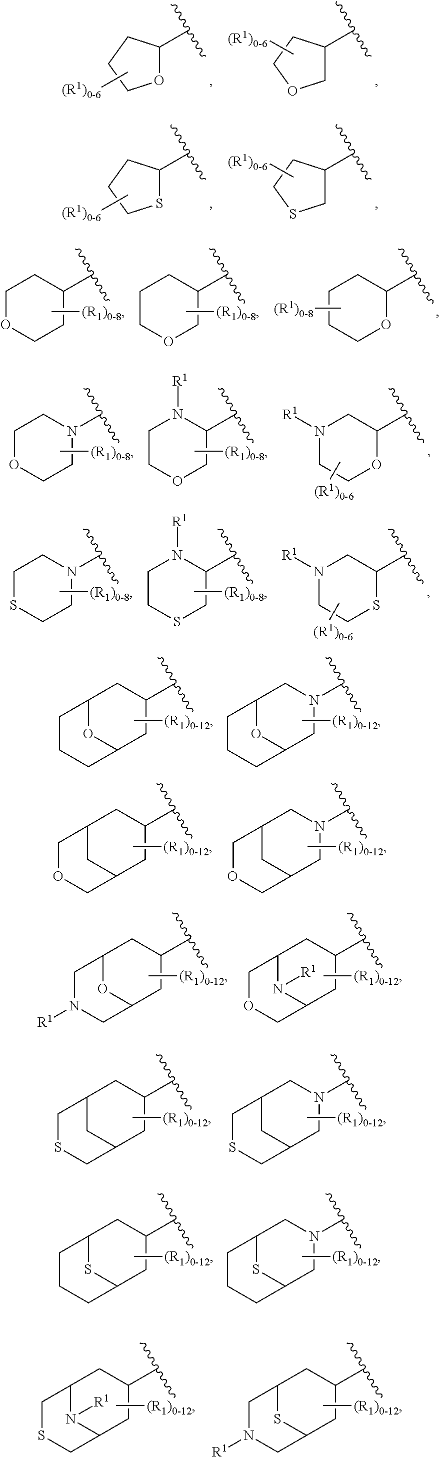

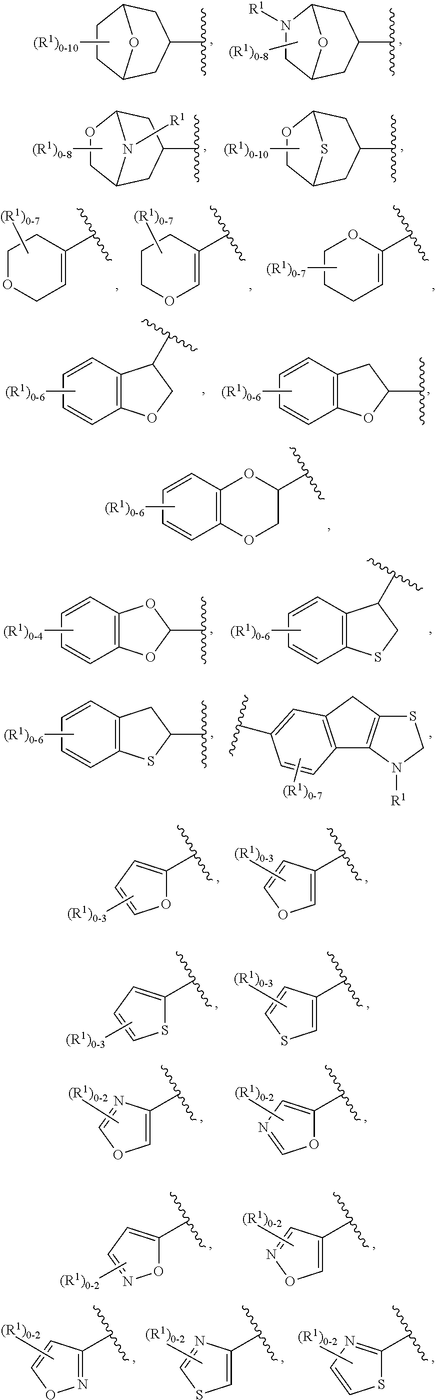

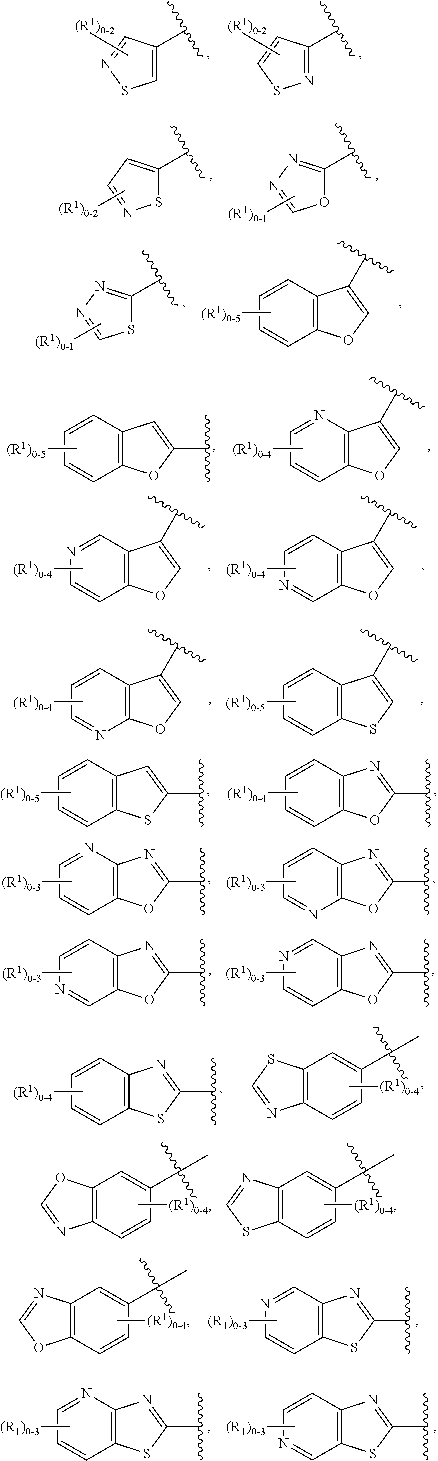

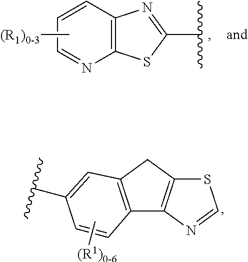

wherein each R¹ is as defined herein. In an embodiment, A and B are each independently a saturated, partially saturated, or unsaturated (e.g., aromatic) derivative of one of the rings described above. In an embodiment, A and B are each independently a stereoisomer of one of the rings described above.
For each of Formulas (I) or (II), in some embodiments, one of A and B is independently a monocyclic heteroaryl or bicyclic heteroaryl, each of which is optionally substituted with one or more R¹. In some embodiments, one of A and B is independently a bicyclic heteroaryl optionally substituted with one or more R¹. In some embodiments, one of A and B is independently a nitrogen-containing heteroaryl optionally substituted with one or more R¹. In some embodiments, one of A and B is independently selected from

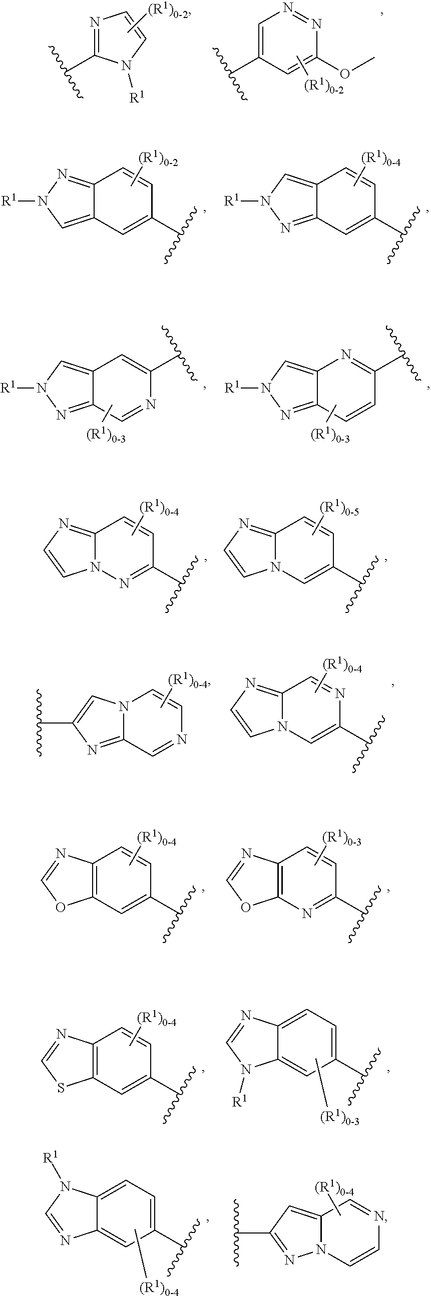

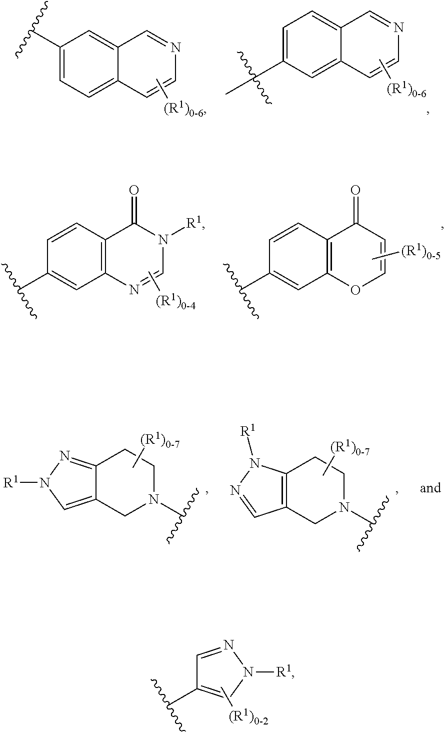

wherein R¹ is as described herein. In some embodiments, one of A and B is independently selected from

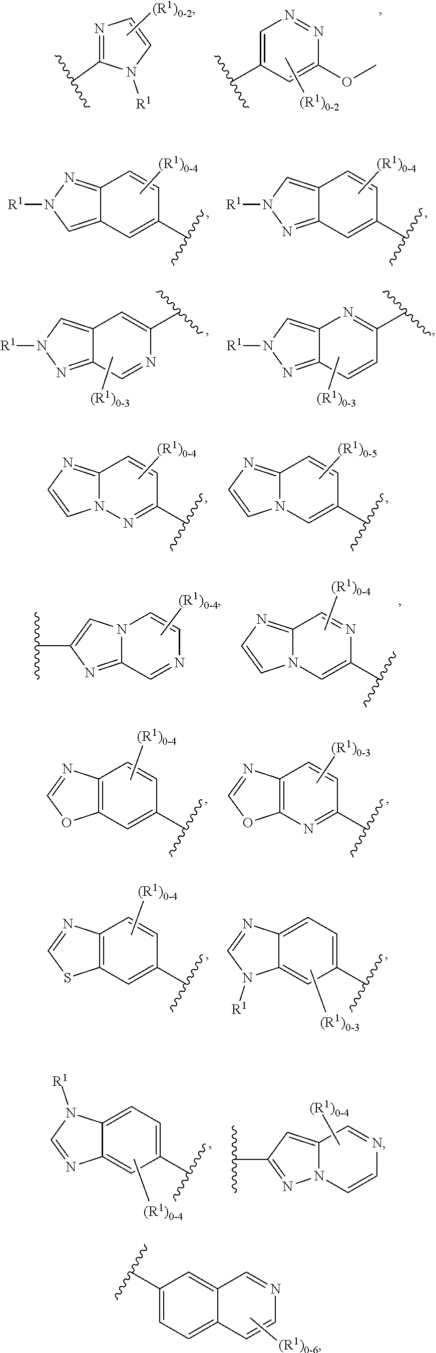

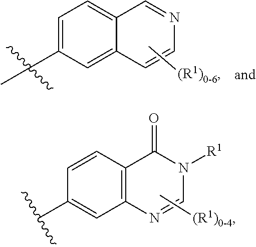

wherein R¹ is as described herein. In some embodiments, one of A and B is independently selected from

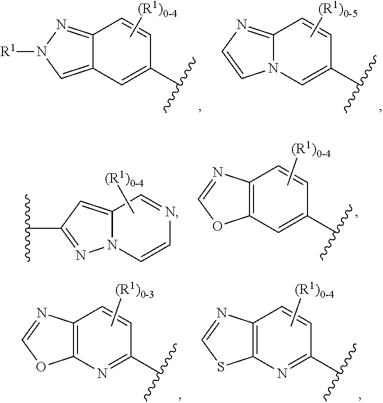

wherein R¹ is as described herein. In some embodiments, one of A and B is independently selected from

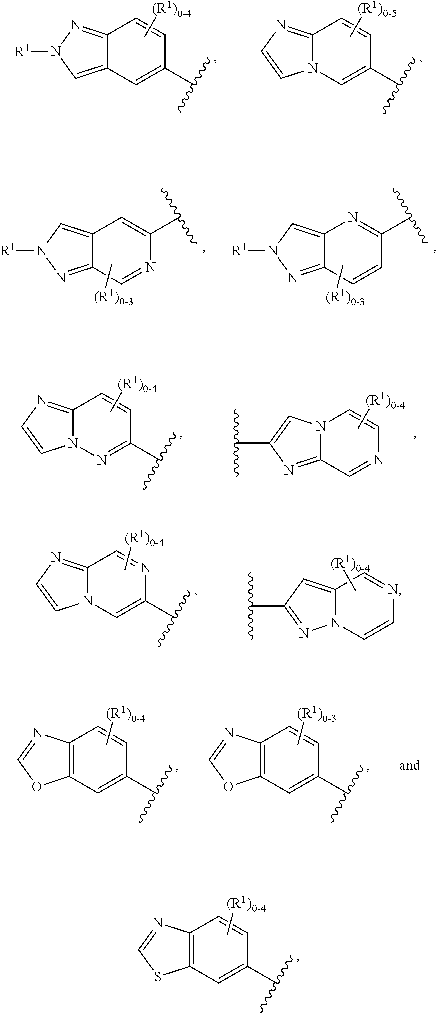

wherein R¹ is as described herein. In some embodiments, one of A and B is independently selected from

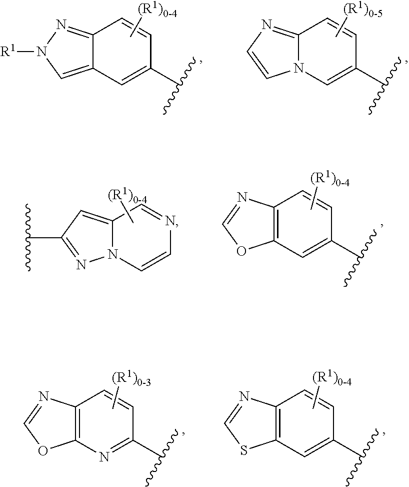

wherein R¹ is as described herein. In some embodiments, A is independently selected from

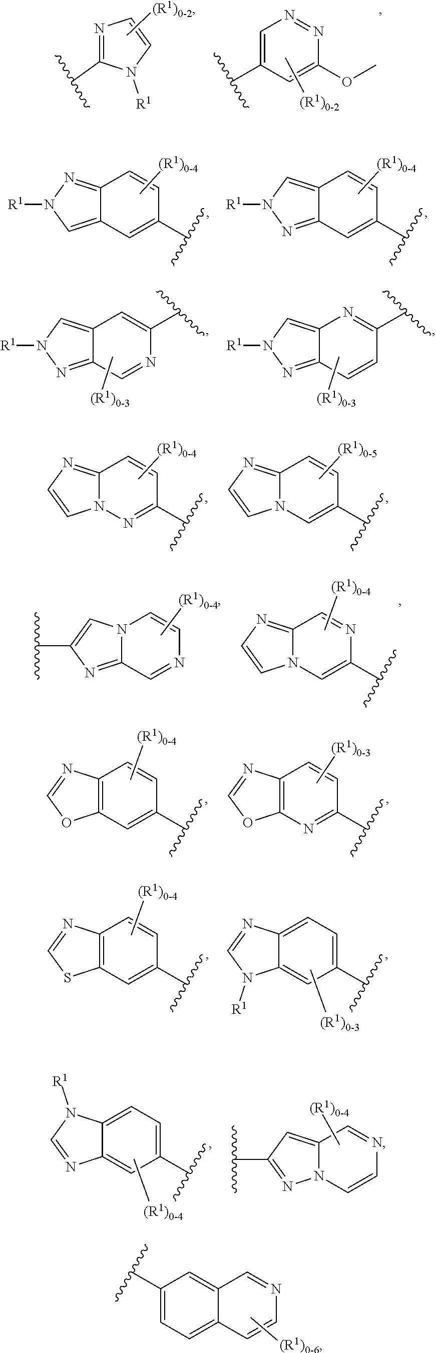

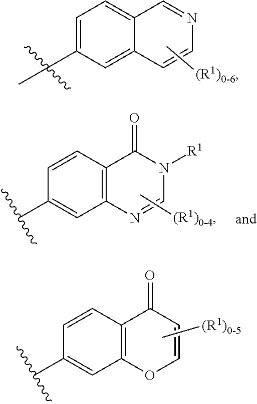

wherein R¹ is as described herein. In some embodiments, A is selected from

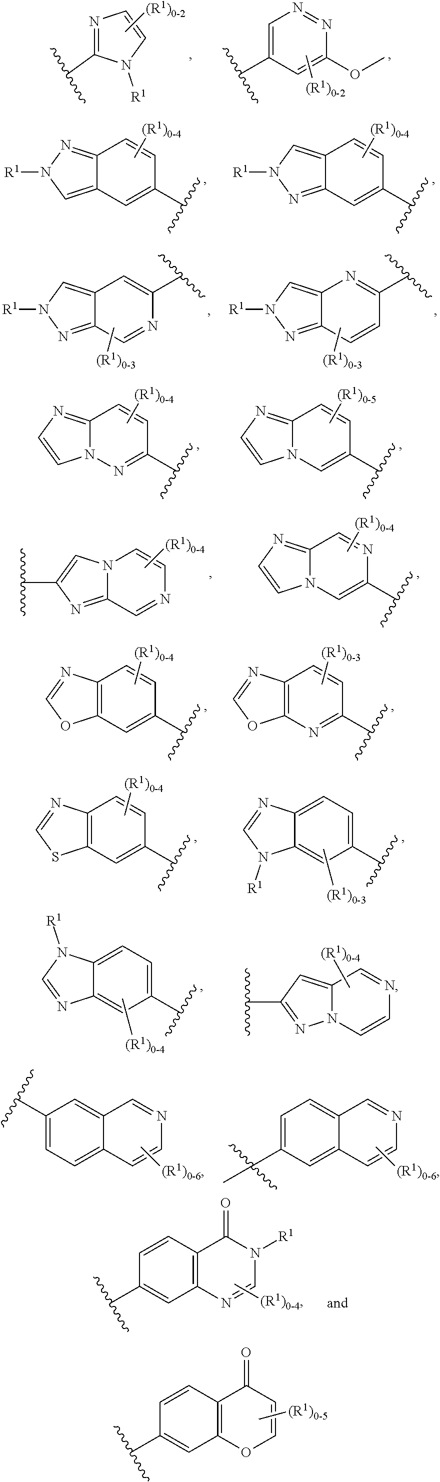

wherein R¹ is as described herein. In some embodiments, A is

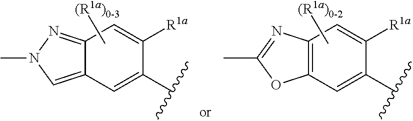

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, B is selected from

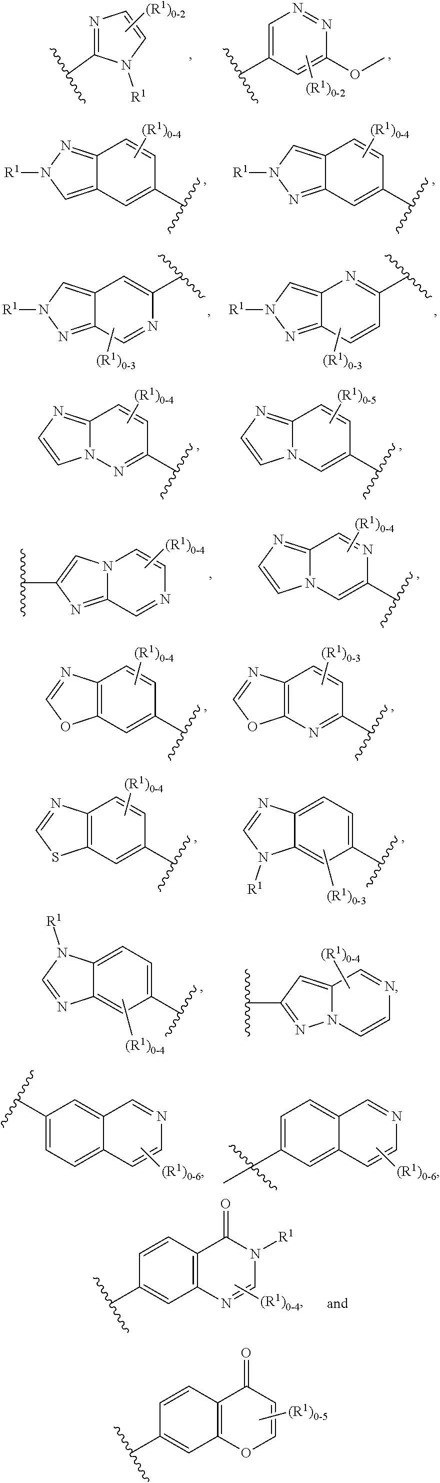

wherein R¹ is as described herein. In some embodiments, B is

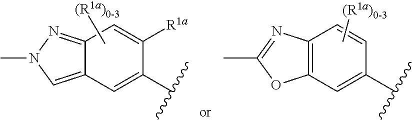

wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

In some embodiments, one of A and B is independently selected from

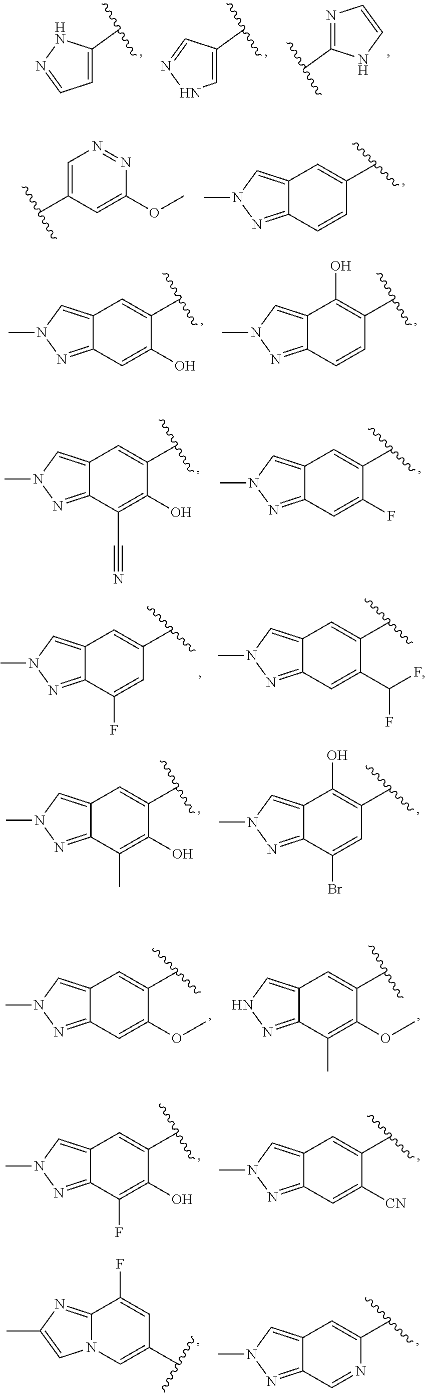

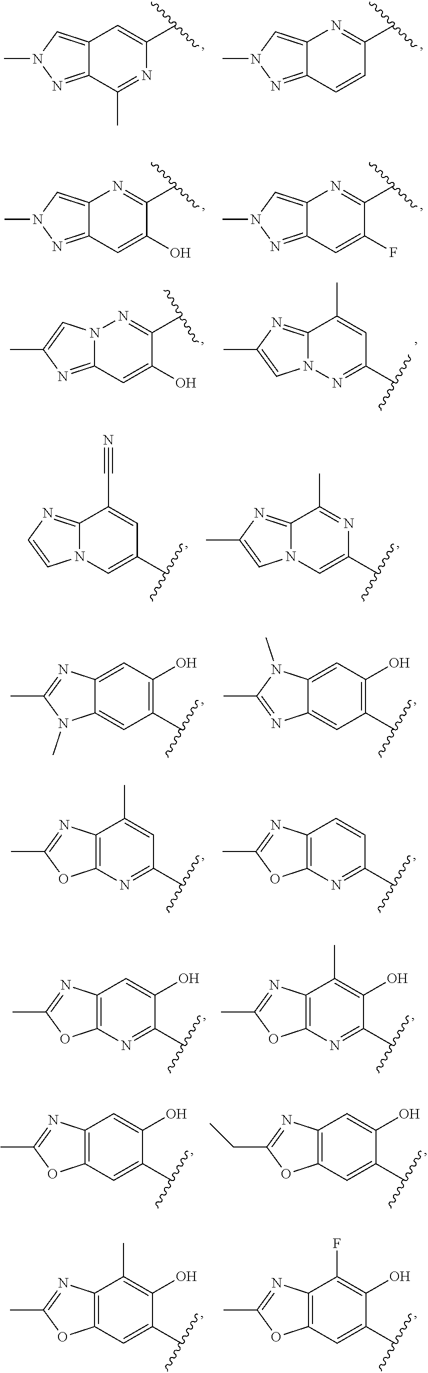

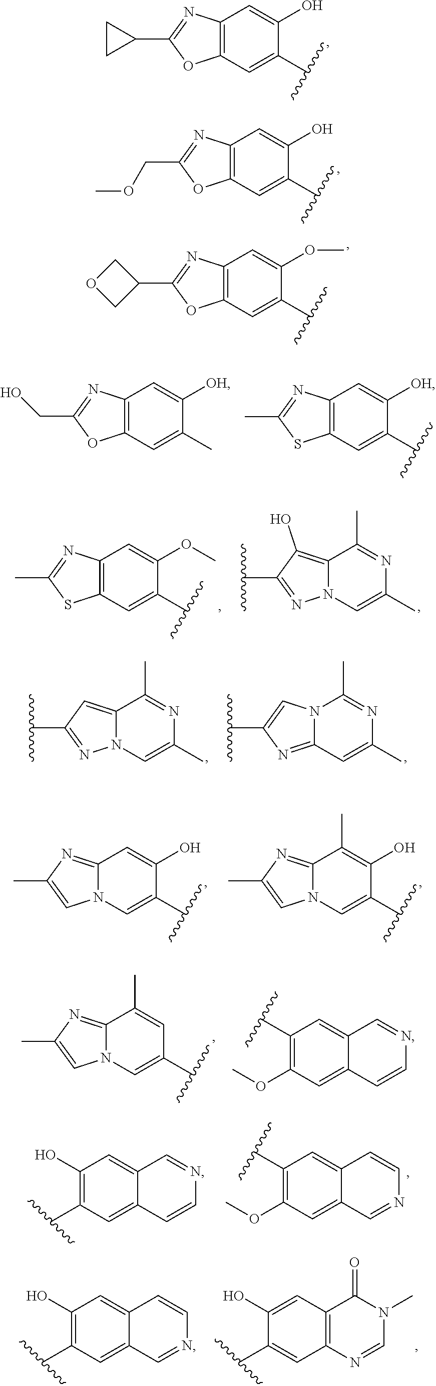

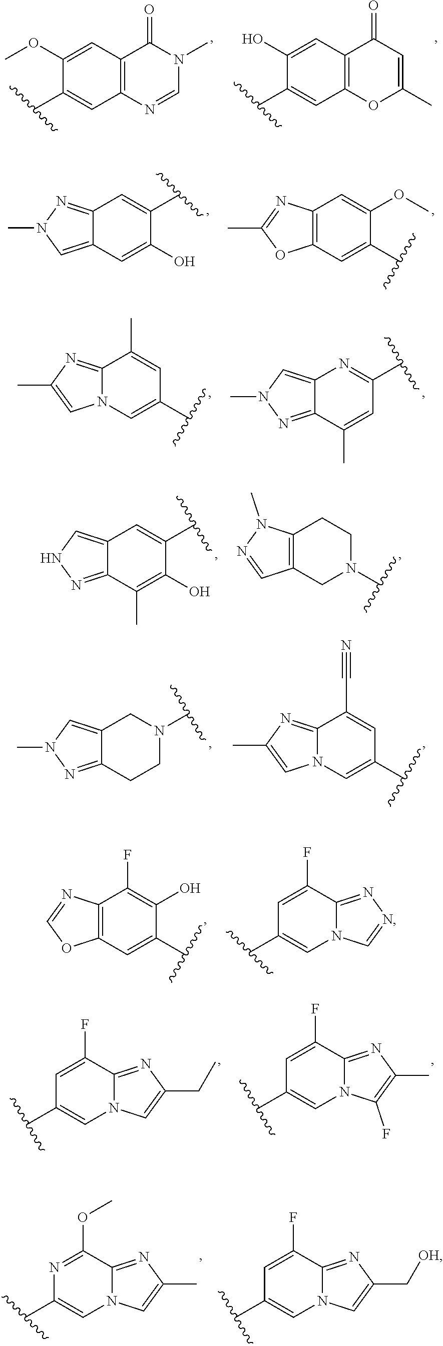

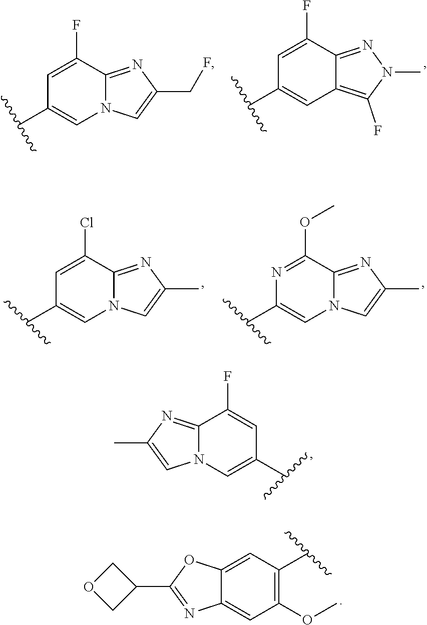 [SMILES: *c1cc(F)c2nc(CF)cn2c1 ; *c1cc(F)c2nn(C)c(F)c2c1 ; *c1cc(Cl)c2nc(C)cn2c1 ; *c1cn2cc(C)nc2c(OC)n1 ; *c1cc(F)c2nc(C)cn2c1 ; *c1cc2oc(C3COC3)nc2cc1OC]

In some embodiments, one of A and B is independently selected from

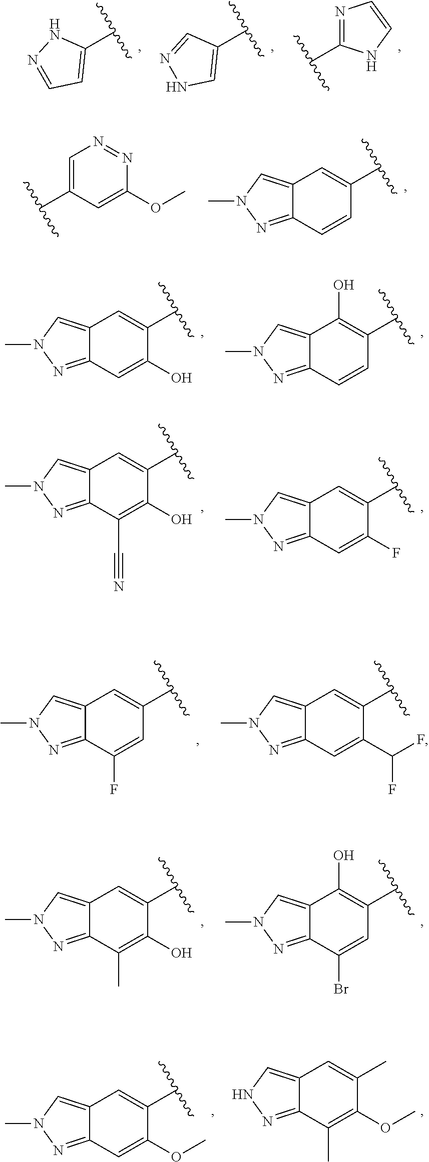

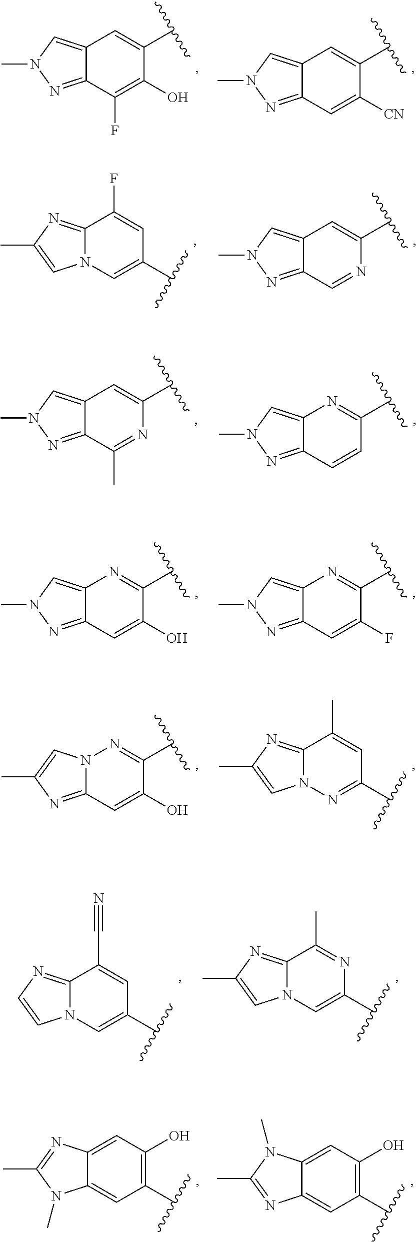

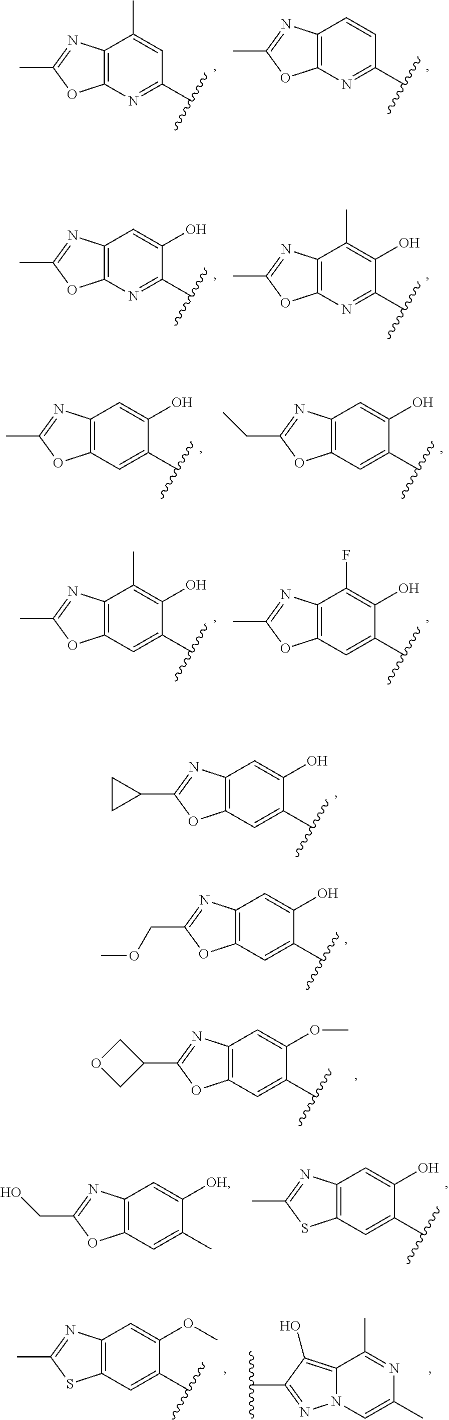

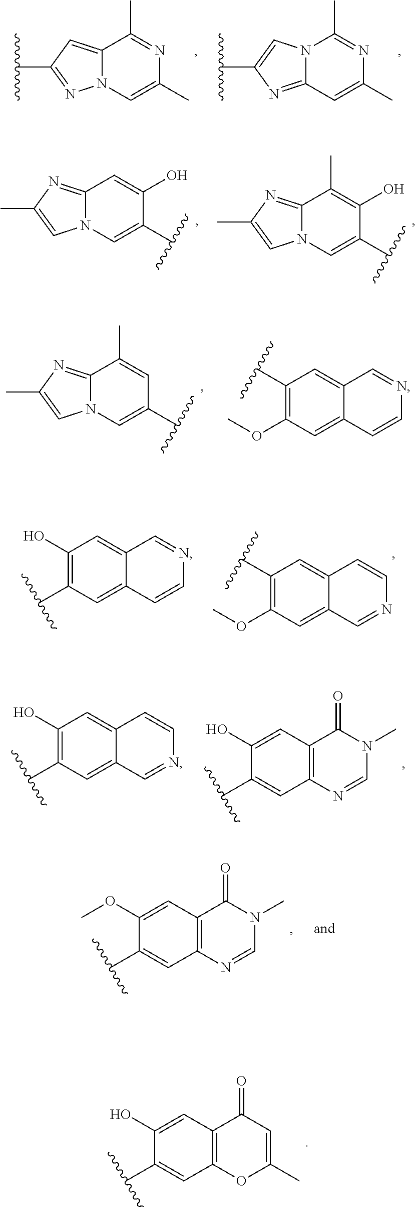

In some embodiments, one of A and B is independently selected from

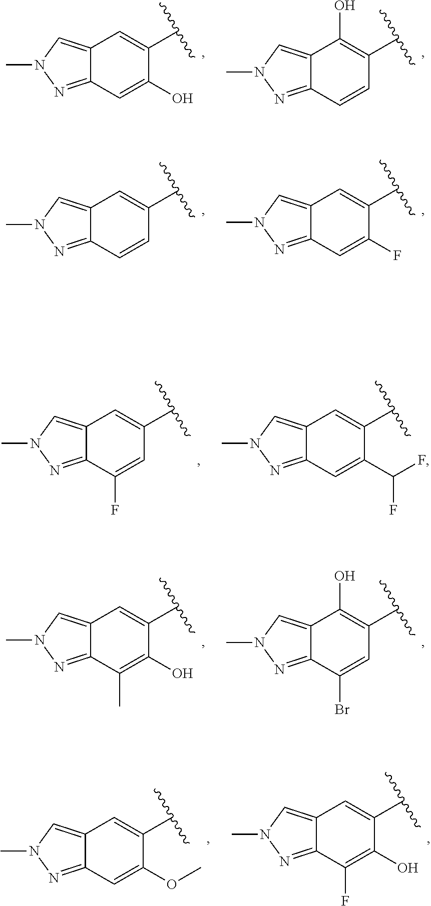

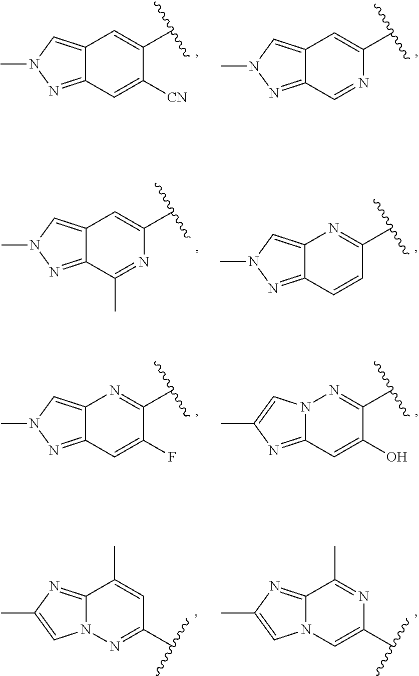

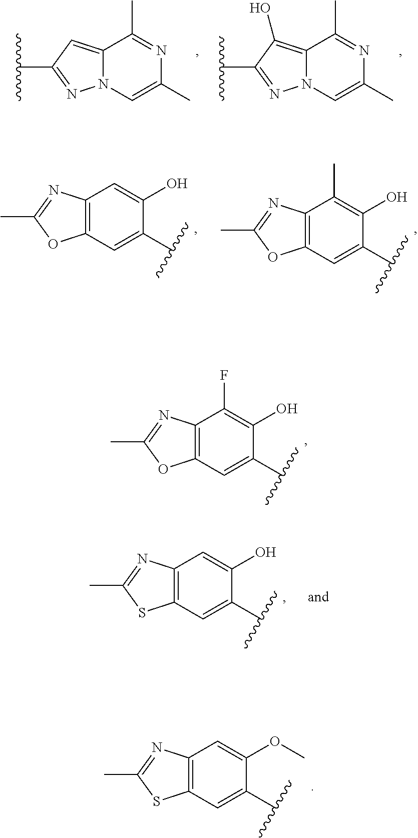

In some embodiments, one of A and B is independently selected from

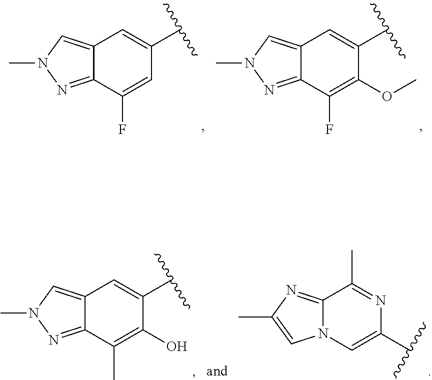

In some embodiments, one In some embodiments, A is selected from

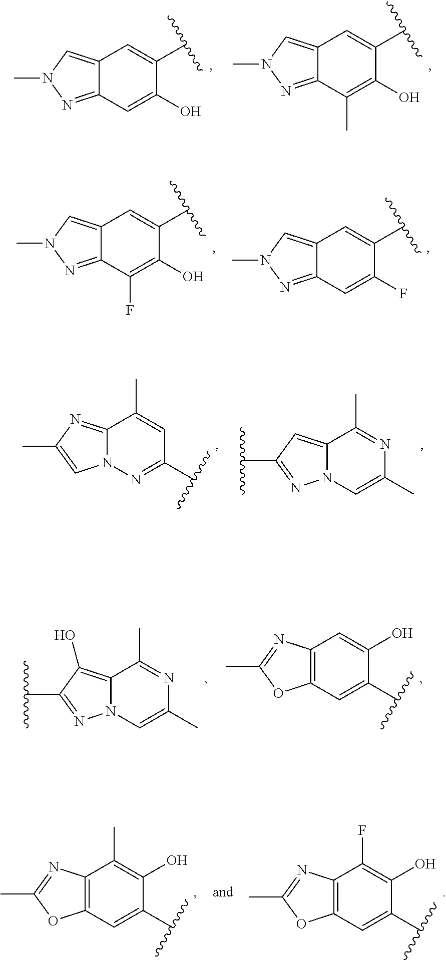

In some embodiments, B is selected from

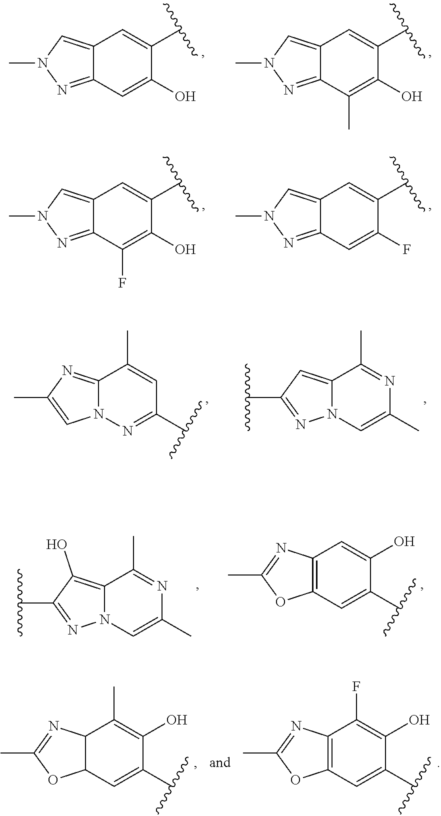

For each of Formulas (I) or (II), in some embodiments, one of A and B is independently a monocyclic heterocyclyl or bicyclic heterocyclyl, each of which is optionally substituted with one or more R¹. In some embodiments, one of A and B is independently a monocyclic heterocyclyl optionally substituted with one or more R¹. In some embodiments, one of A and B is independently a bicyclic heterocyclyl optionally substituted with one or more R¹. In some embodiments, one of A and B is independently a nitrogen-containing heterocyclyl optionally substituted with one or more R¹. In some embodiments, one of A and B is independently selected from

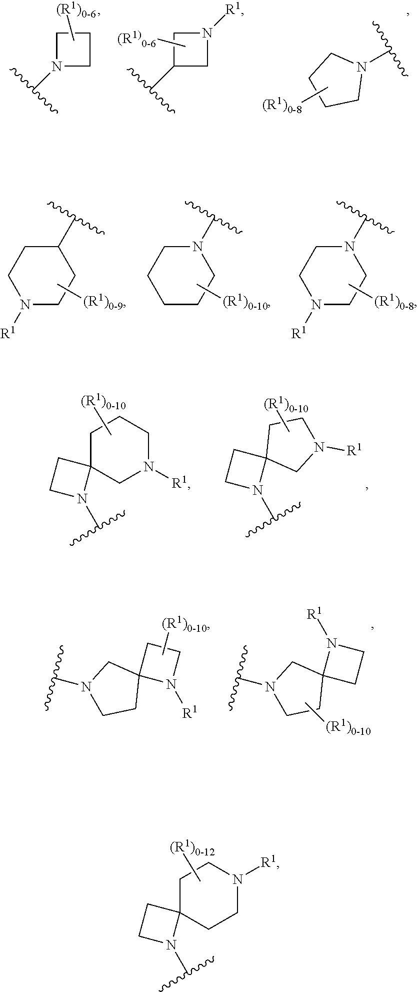

wherein R¹ is as described herein. In some embodiments, A is selected from

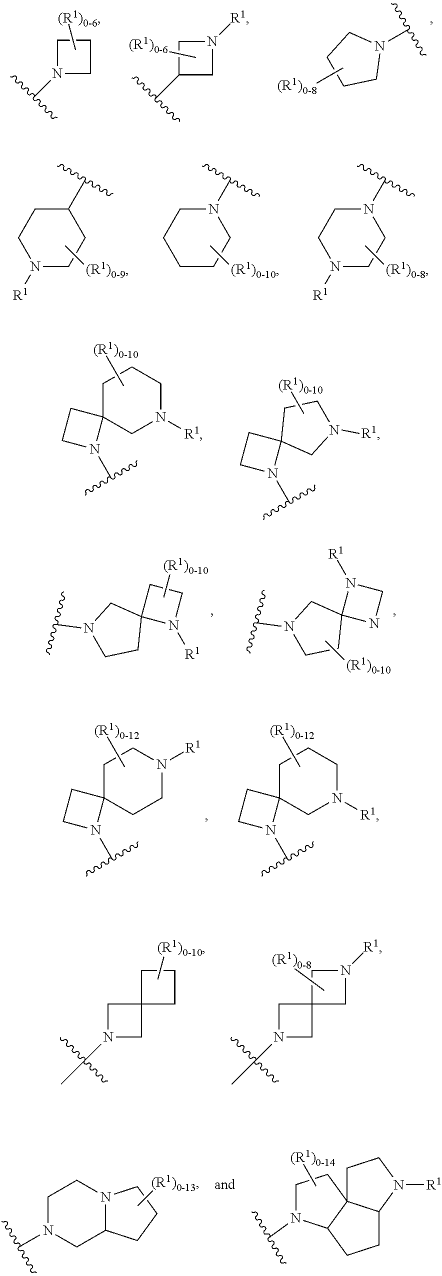

wherein R¹ is as described herein. In some embodiments, B is selected from,

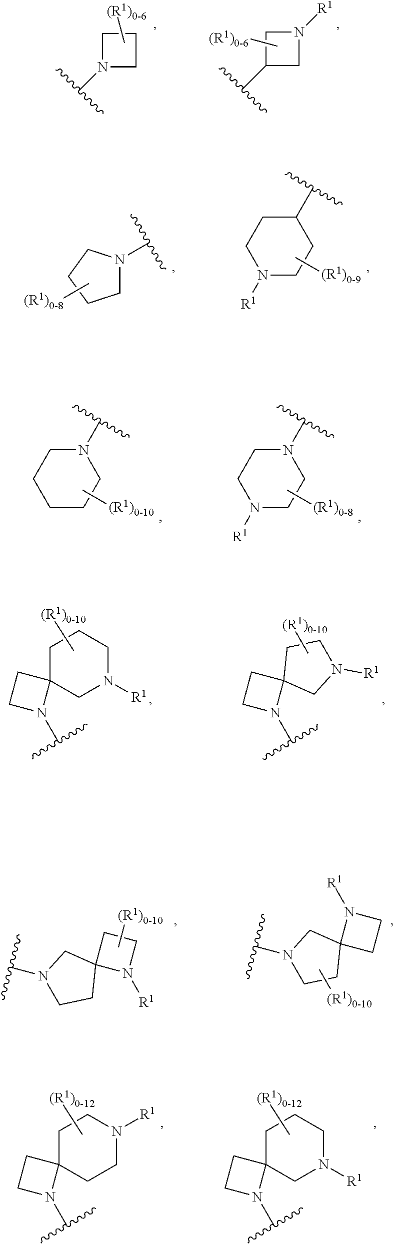

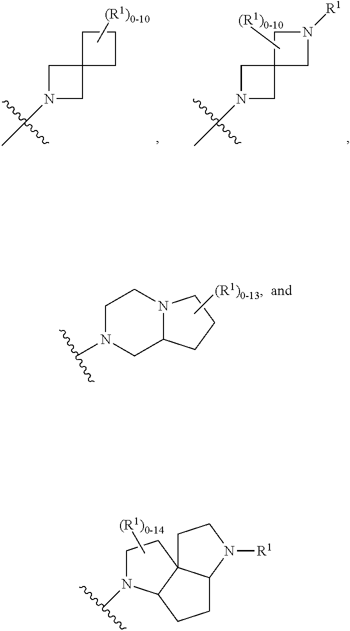

wherein R¹ is as described herein.

In some embodiments, one of A and B is independently selected from

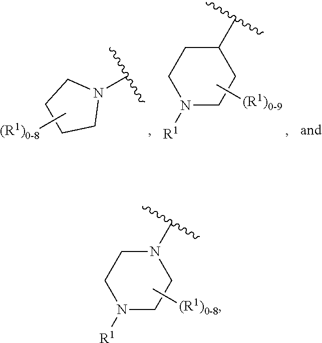

wherein R¹ is as described herein. In some embodiments, one of A and B is

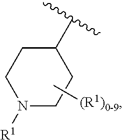

wherein R¹ is as described herein. In some embodiments, A is

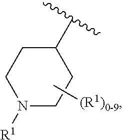

wherein R¹ is as described herein. In some embodiments, B is

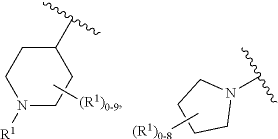

wherein R¹ is as described herein. In some embodiments, one of A and B is independently and R¹ is as described herein. In some embodiments, one of A and B is independently

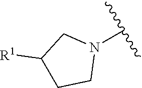

and R¹ is as described herein. In some embodiments, one of A and B is independently selected from

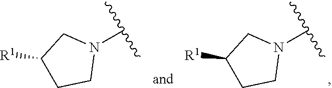

and R¹ is as described herein.

In some embodiments, one of A and B is independently

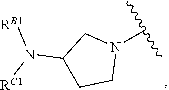

and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹. In some embodiments, R^{B1} is hydrogen and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹. In some embodiments, one of A and B is independently selected from

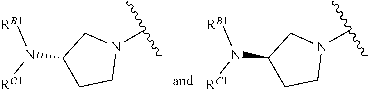

and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹. In some embodiments, R^{B1} is hydrogen and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.
In some embodiments, one of A and B is independently is selected

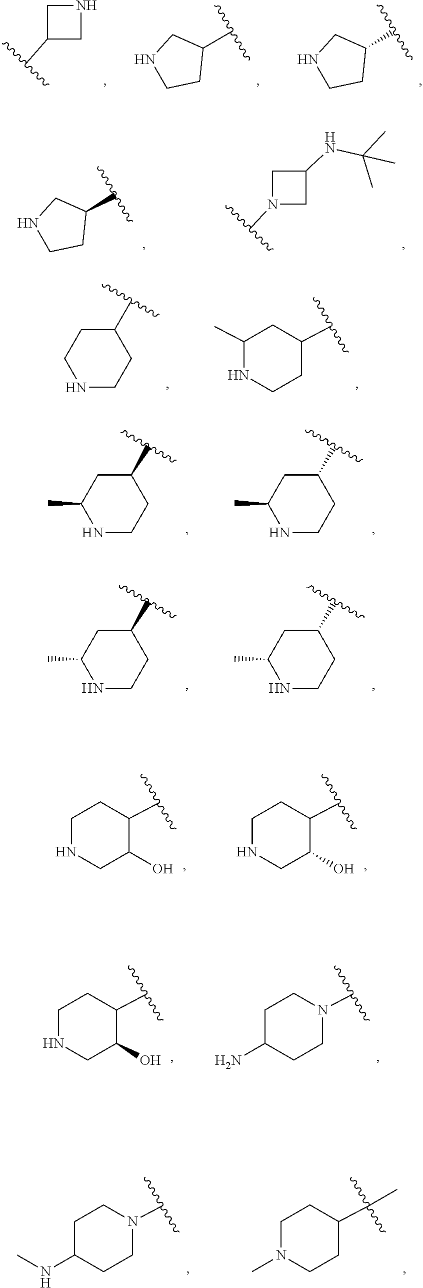

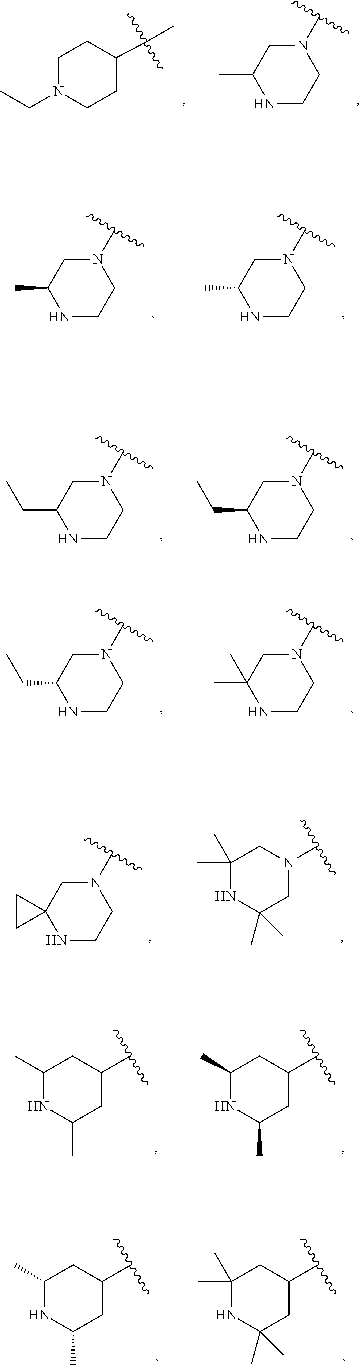

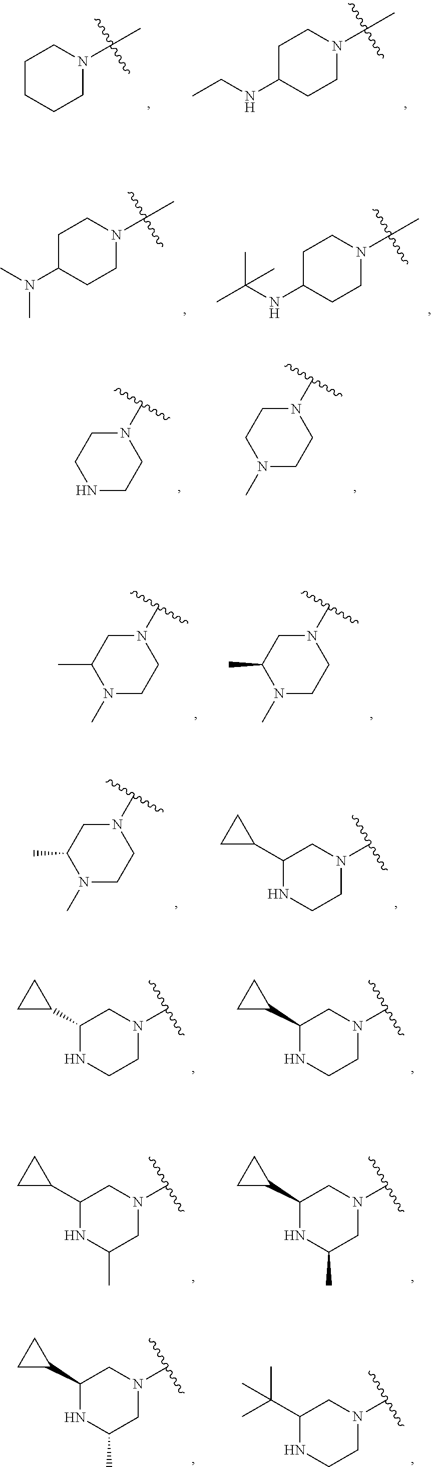

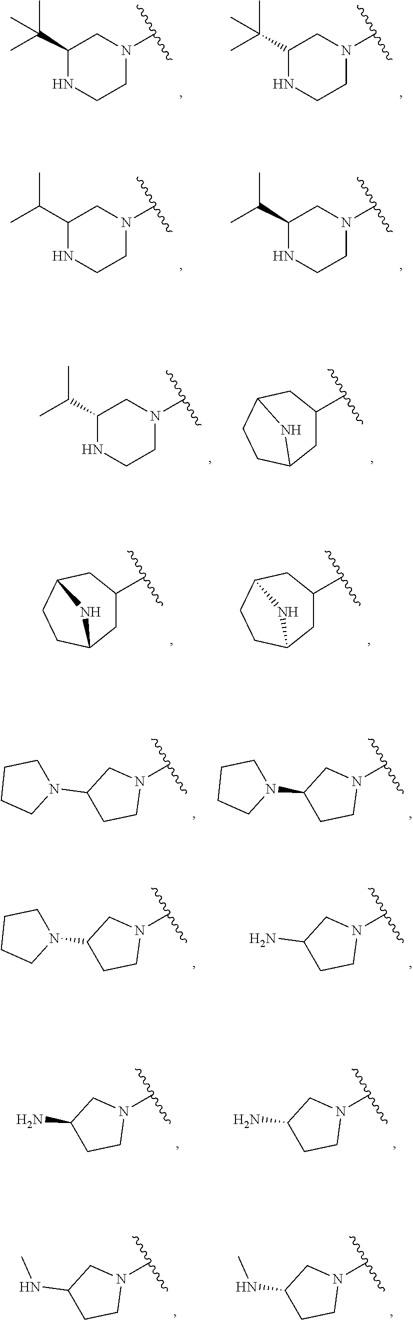

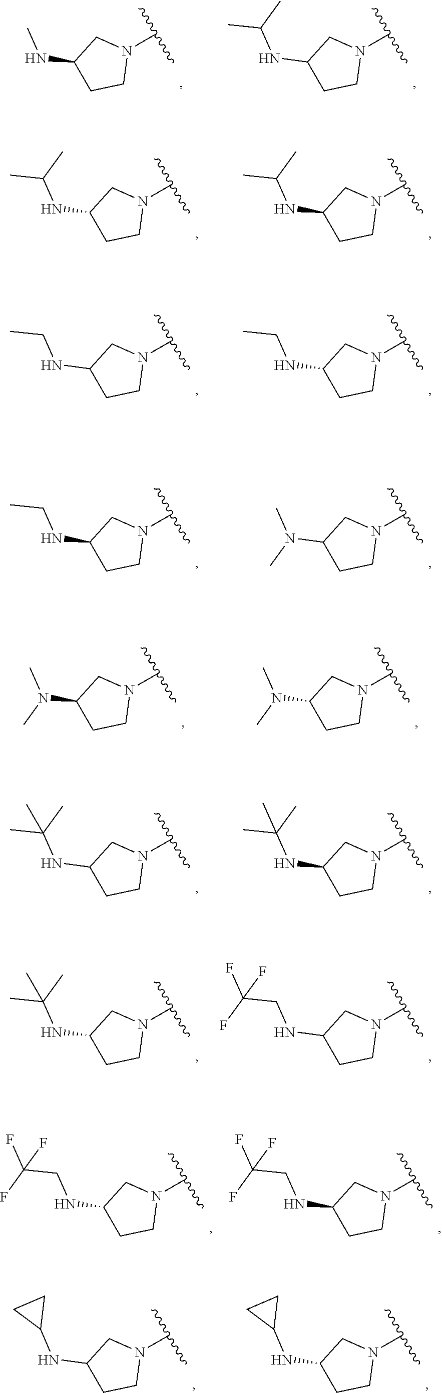

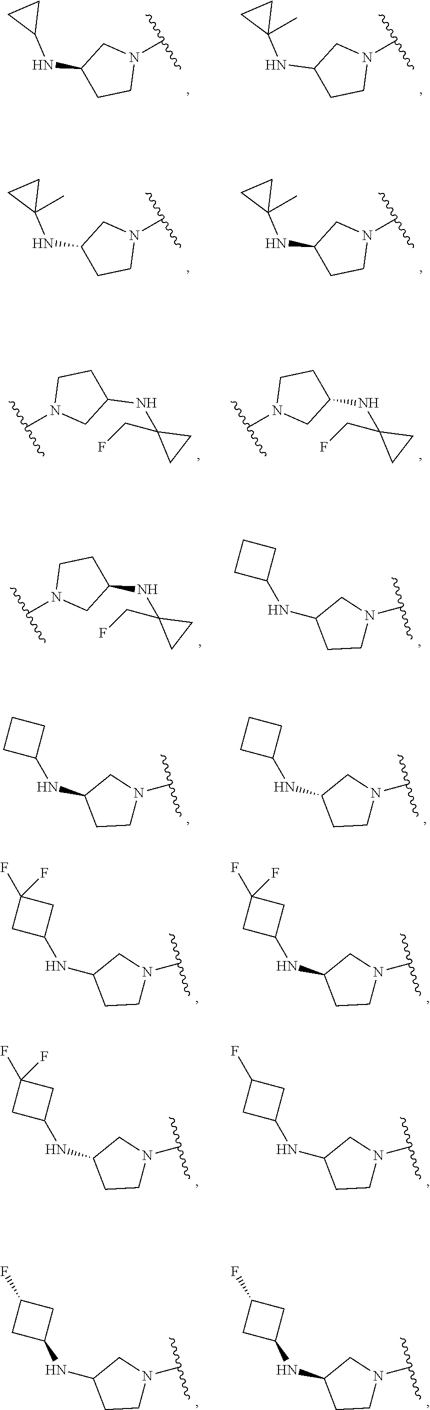

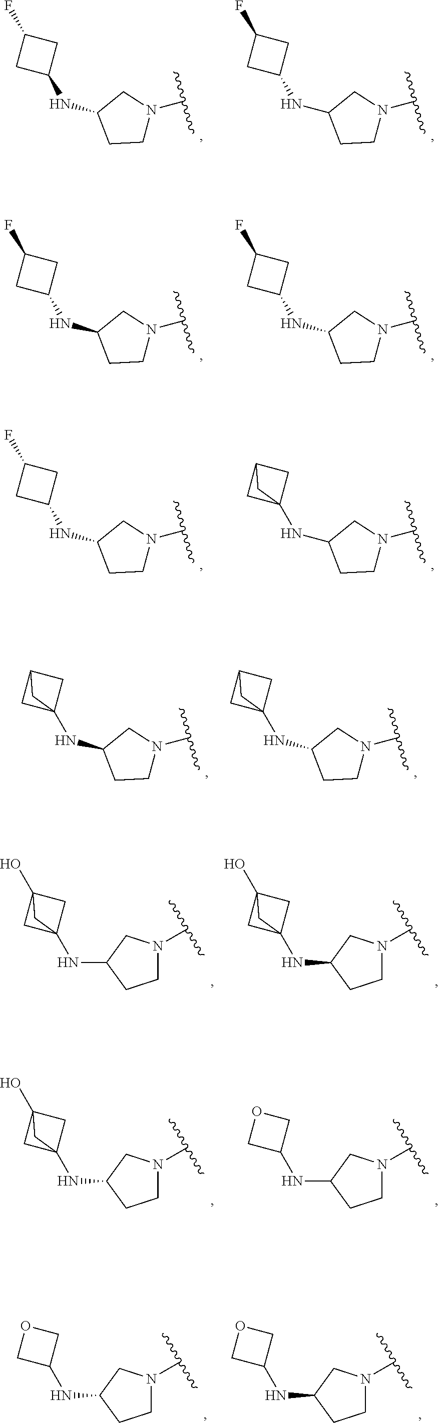

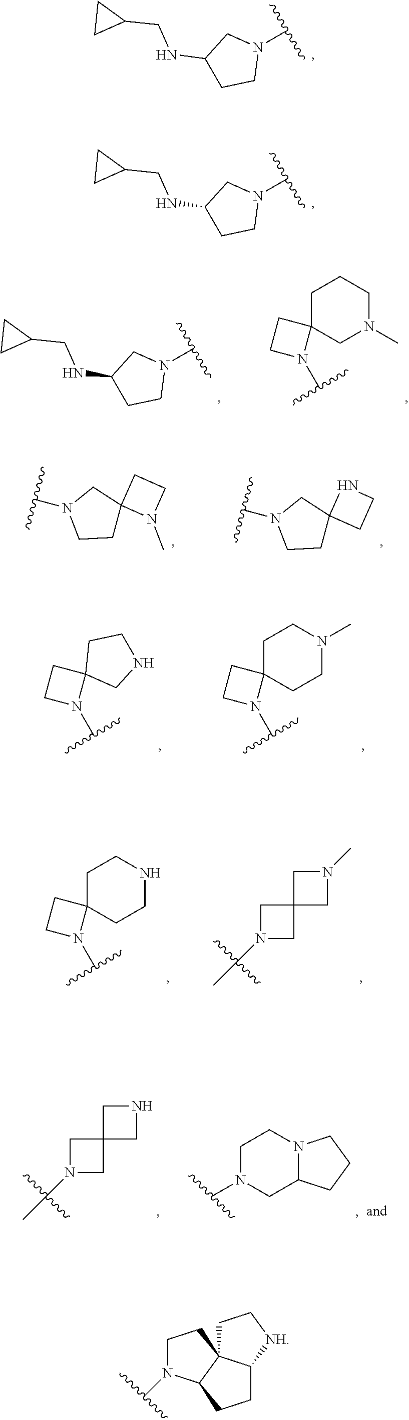

In some embodiments, one of A and B is independently is selected from

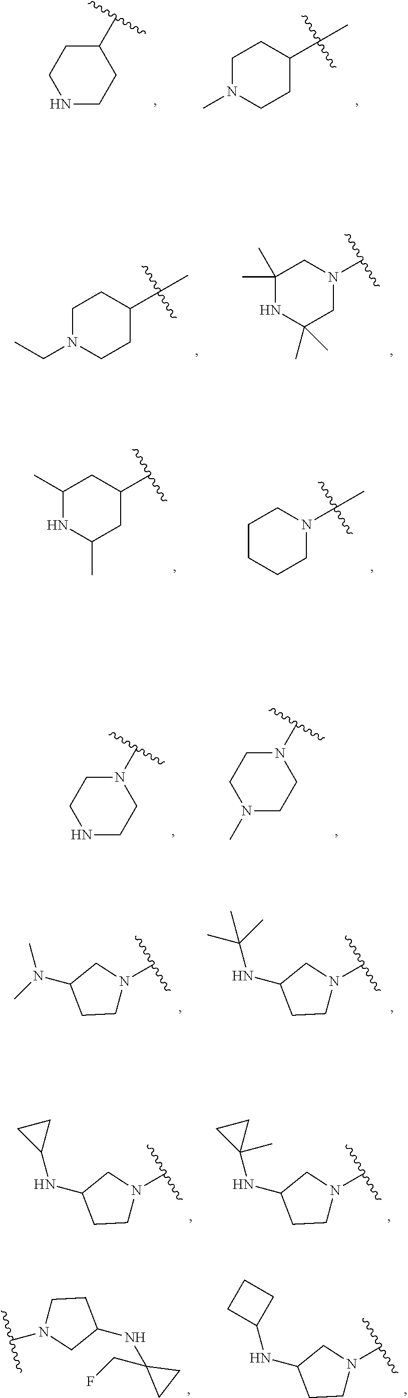

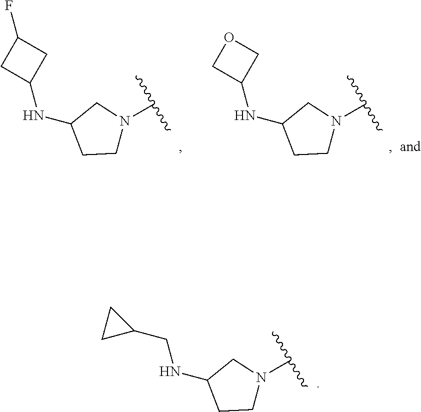 [SMILES: *N1CCC(NC2CC(F)C2)C1 ; *N1CCC(NC2COC2)C1 ; *N1CCC(NCC2CC2)C1]

In some embodiments, wherein A is selected from

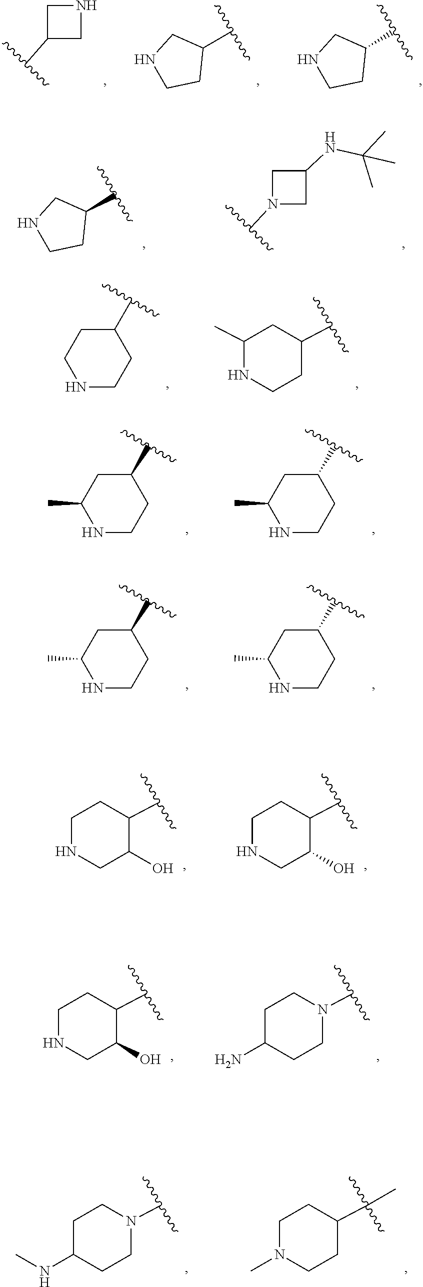

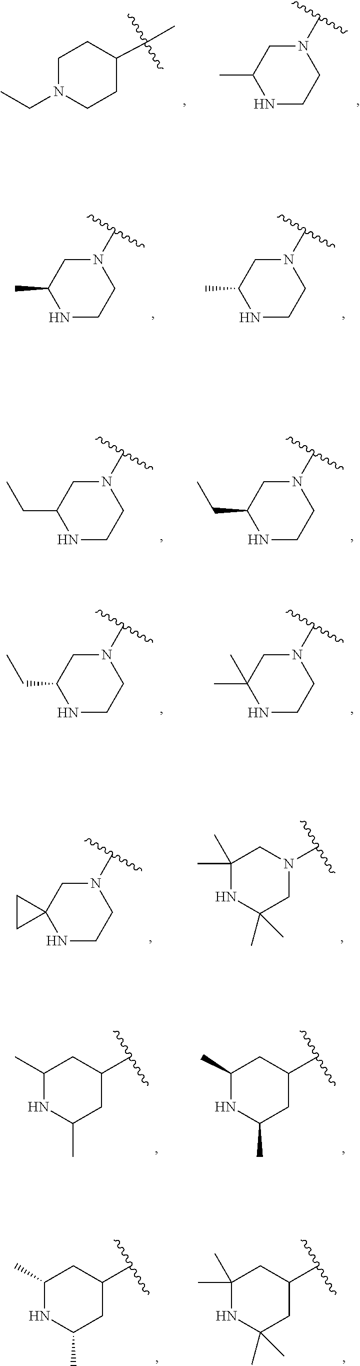

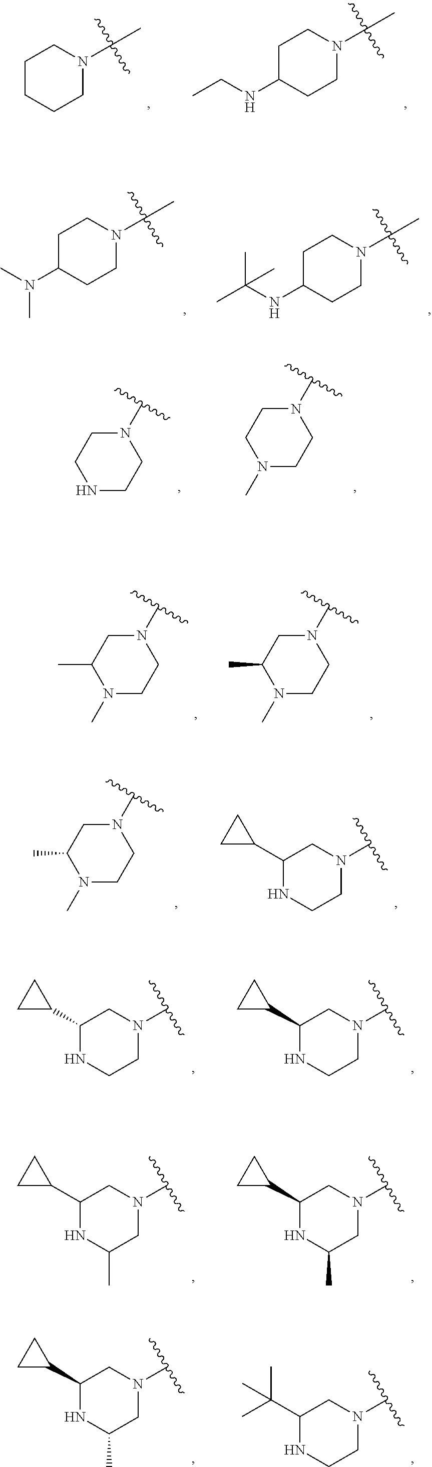

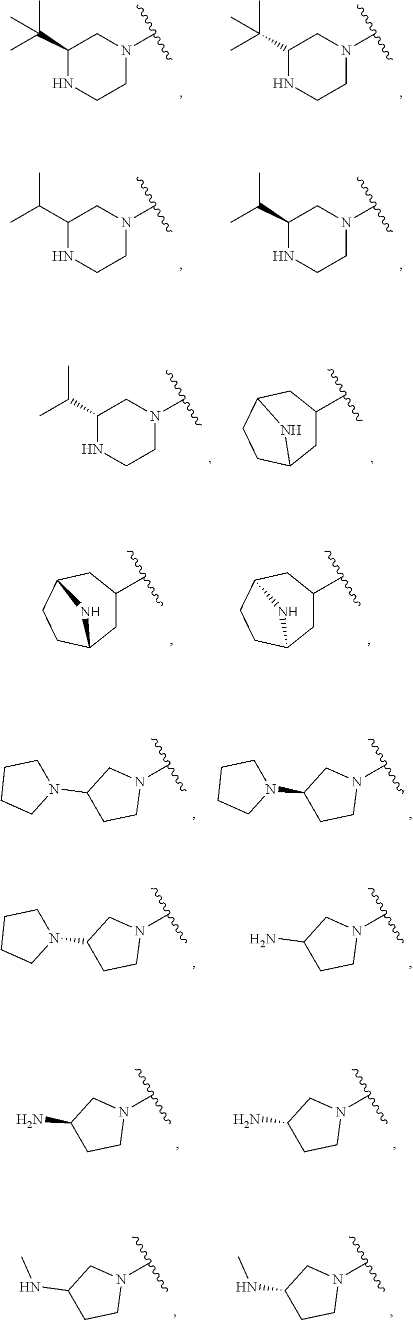

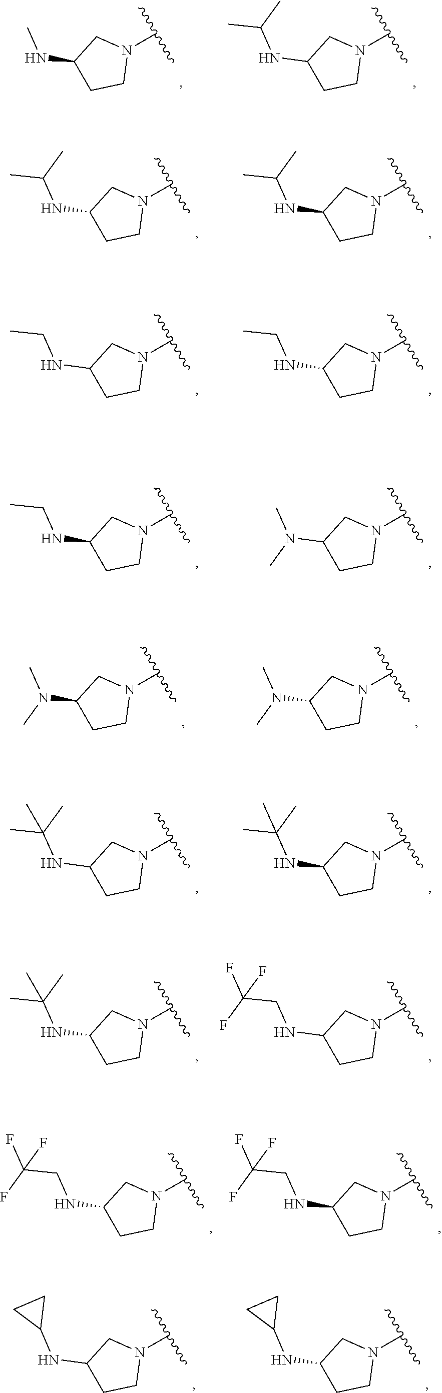

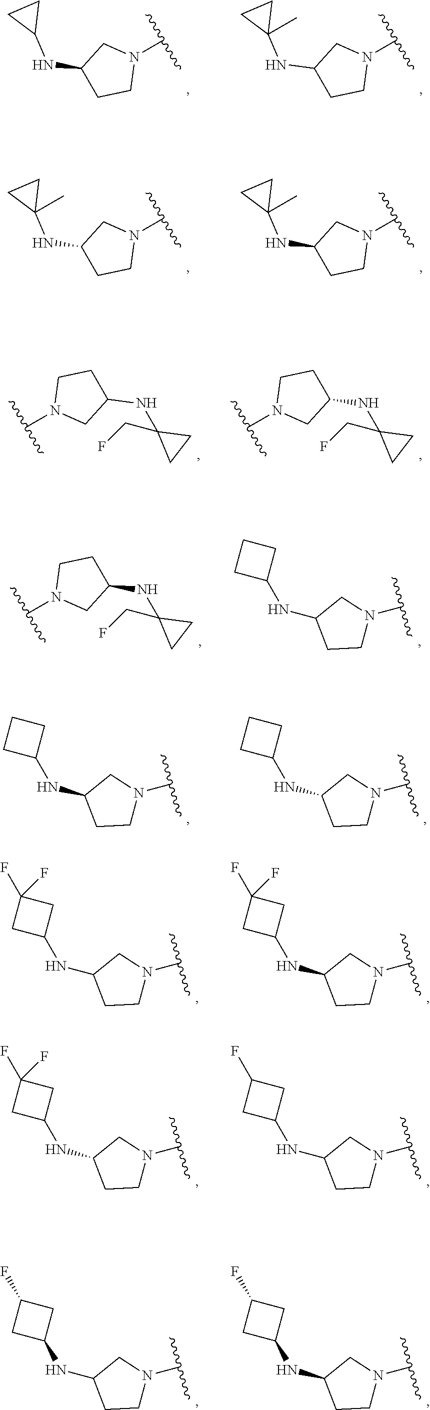

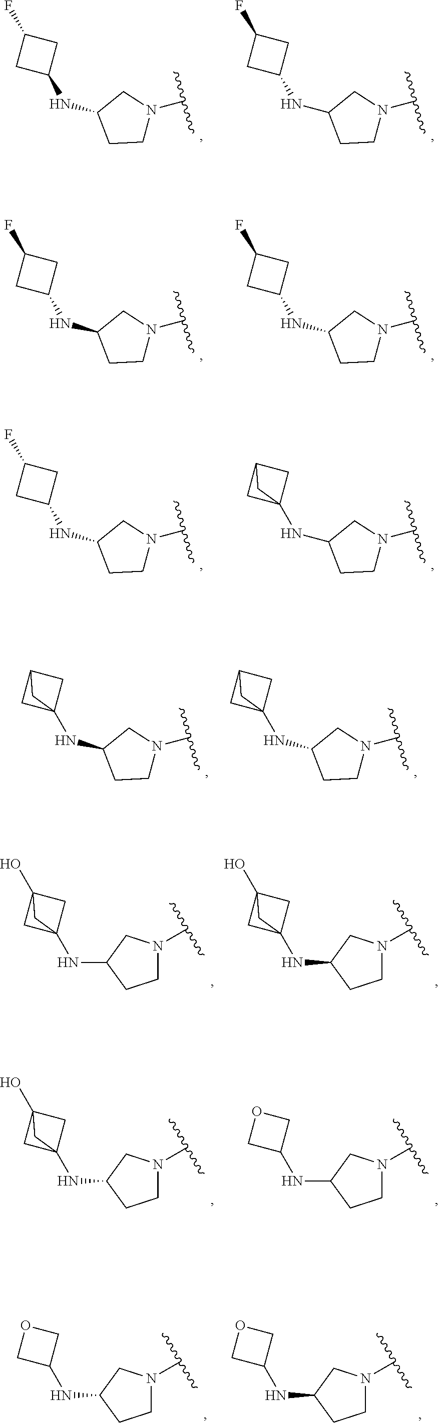

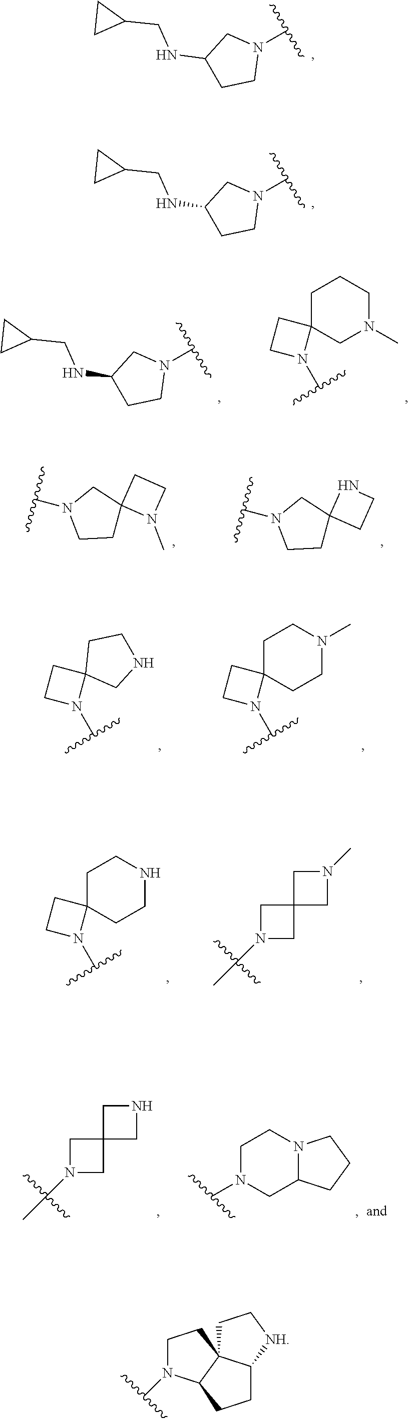

In some embodiments wherein B is selected from

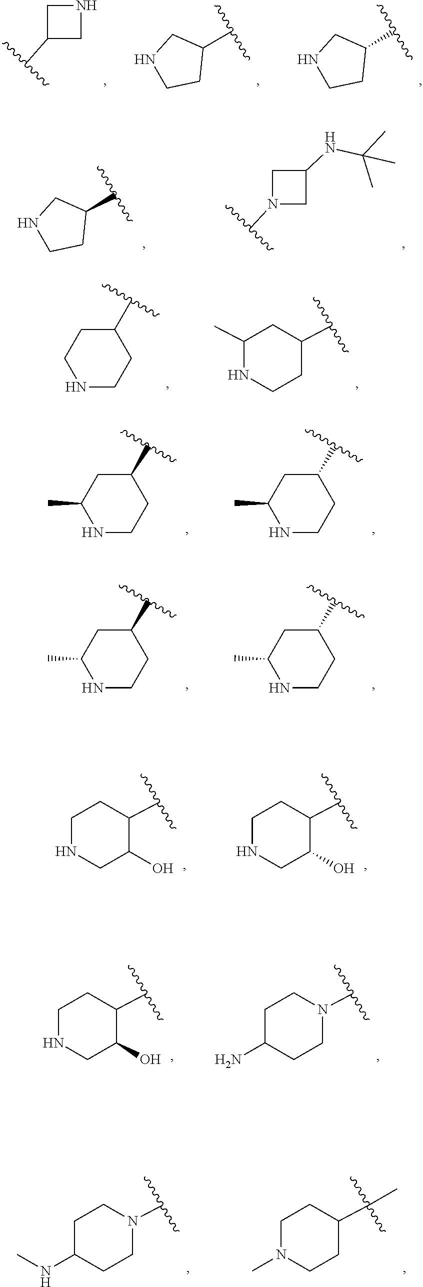

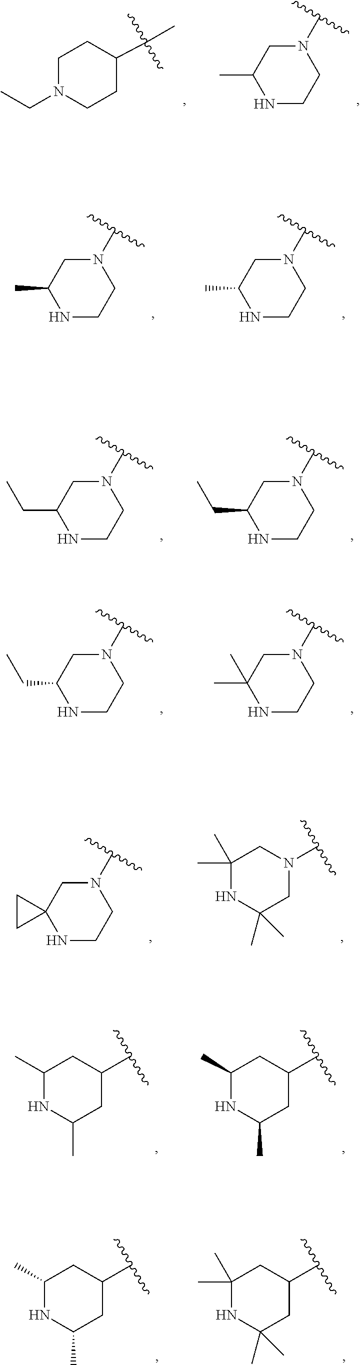

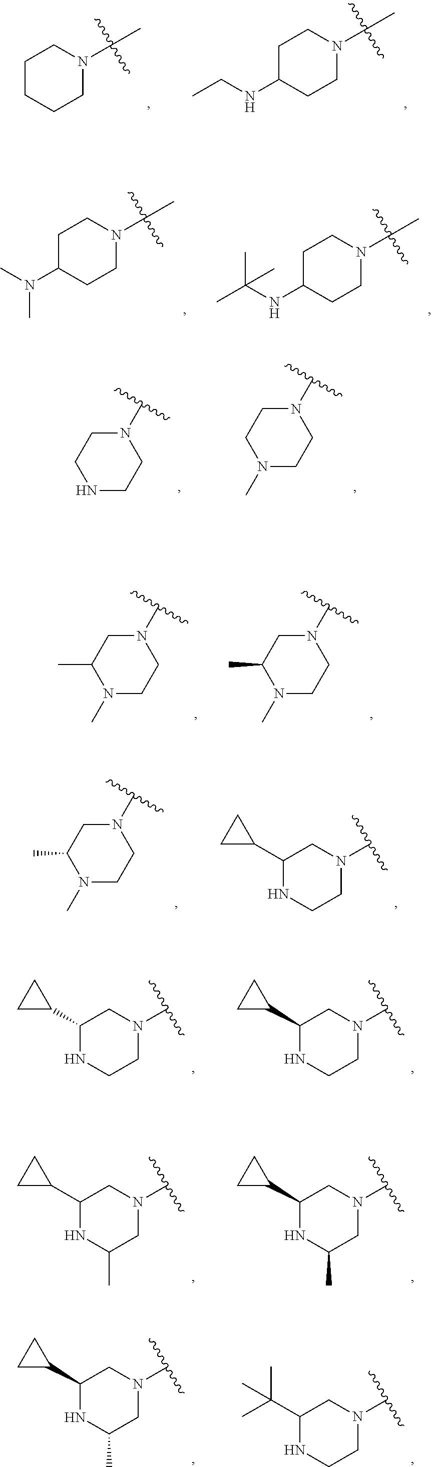

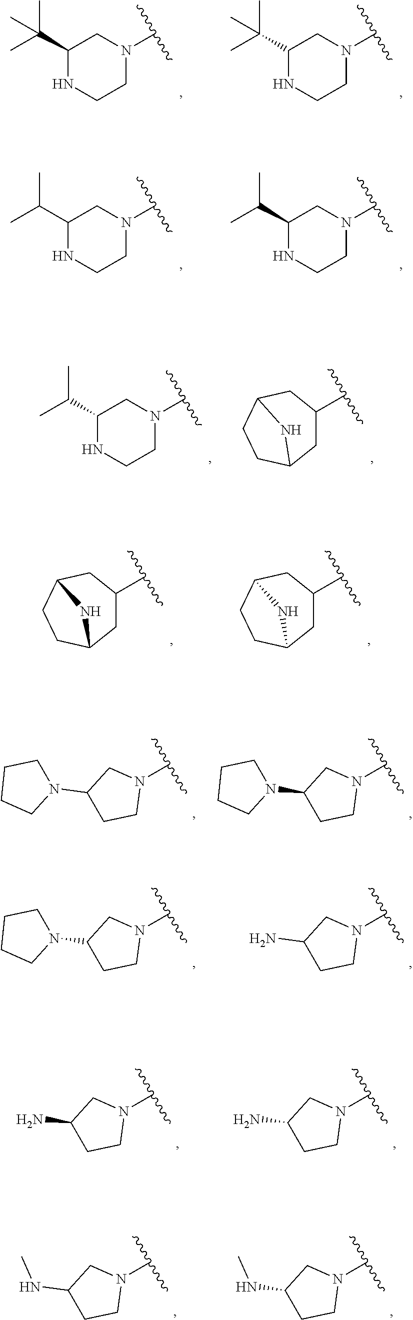

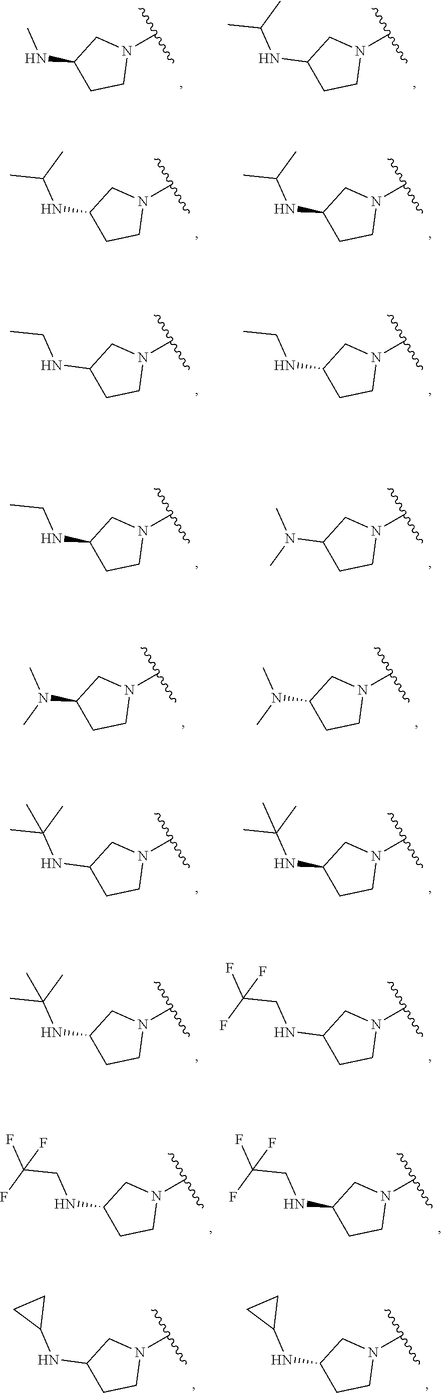

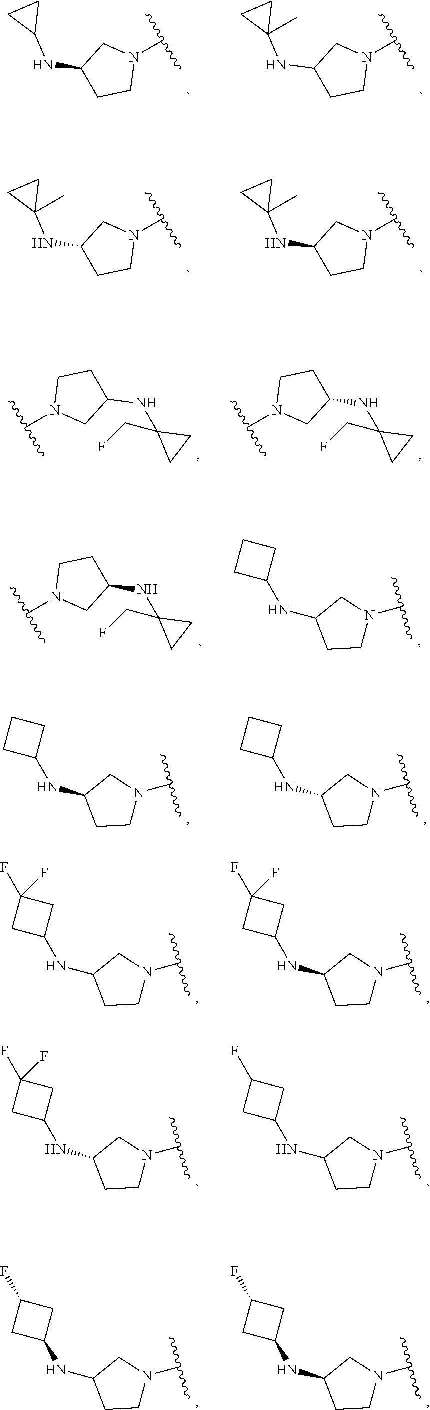

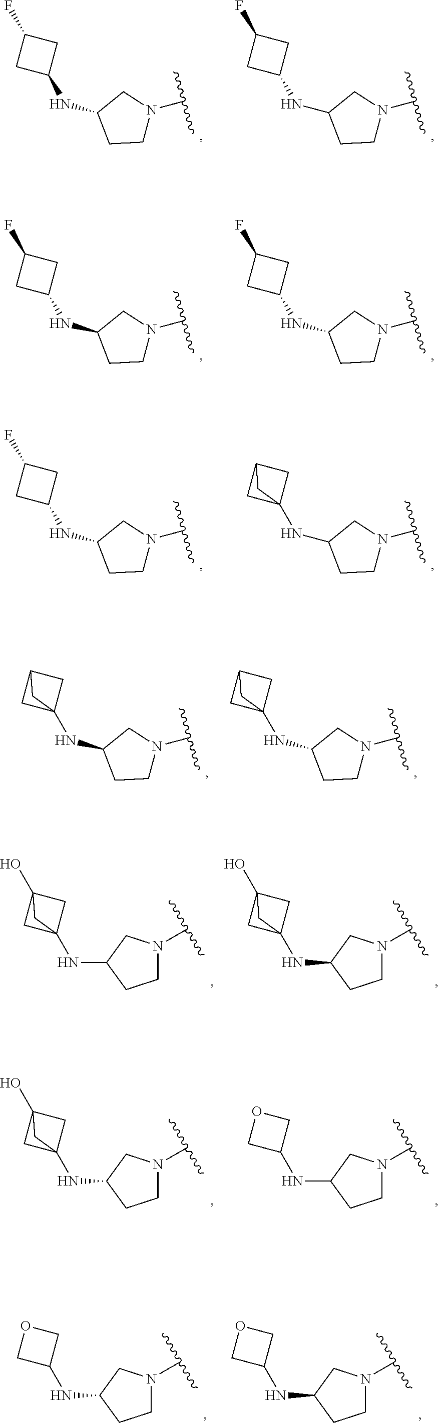

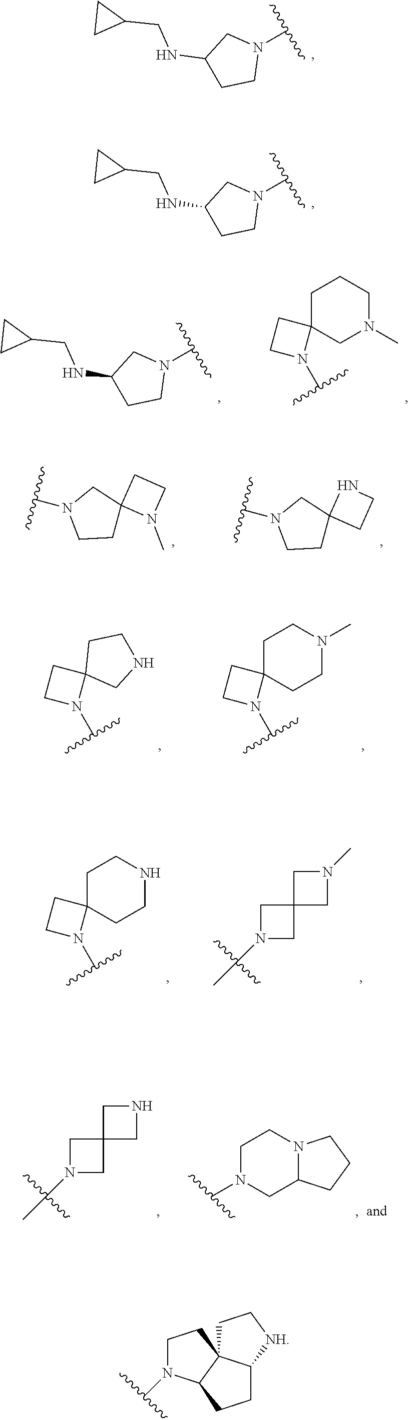

In some embodiments, one of A and B is

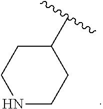 [SMILES: *C1CCNCC1]

In some embodiments, A is

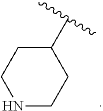 [SMILES: *C1CCNCC1]

In some embodiments, B is

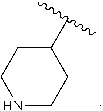 [SMILES: *C1CCNCC1]

As generally described for Formulas (I) and (II), each of L¹ and L² may independently be absent or refer to a C₁-C₆-alkylene, C₁-C₆-heteroalkylene, —O—, —C(O)—, —N(R⁸)—, —N(R′)C(O)—, or —C(O)N(R⁸)— group, wherein each alkylene and heteroalkylene is optionally substituted with one or more R⁹. In some embodiments, L¹ is absent or C₁-C₆-heteroalkylene. In some embodiments, L¹ is absent. In some embodiments, L¹ is C₁-C₆-heteroalkylene (e.g., —N(CH₃)—). In some embodiments, L² is absent or C₁-C₆-heteroalkylene. In some embodiments, L² is absent. In some embodiments, L² is C₁-C₆-heteroalkylene (e.g., —N(CH₃)—).

As generally described for Formulas (I) and (II), each R² and R³ is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}. In some embodiments, R² is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}. In some embodiments, R² is C₁-C₆-alkyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —R^{B}C(O)R^{D}, —C(O)NR^{B}R^{C}, —C(O)R^{D}, —C(O)OR^{D}. In some embodiments, R² is C₁-C₆-alkyl, C₁-C₆-heteroalkyl, halo, cyano, cycloalkyl, heterocyclyl, aryl, heteroaryl, —OR^{A}, or —C(O)NR^{B}R^{C}. In some embodiments, R² is C₁-C₆-alkyl. In some embodiments, R² is C₁-C₆-heteroalkyl. In some embodiments, R² is halo (e.g., chloro or fluoro). In some embodiments, R² is cyano. In some embodiments, R² is cycloalkyl (e.g., cyclopropyl or cyclobutyl). In some embodiments, R² is heterocyclyl. In some embodiments, R² is heteroaryl. In some embodiments, R² is —OR^{A}. In some embodiments, R² is —C(O)NR^{B}R^{C}.

In some embodiments, R³ is C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}. In some embodiments, R³ is C₁-C₆-alkyl, C₁-C₆-heteroalkyl, C₁-C₆-haloalkyl, halo, cyano, cycloalkyl, heterocyclyl, aryl, heteroaryl, —OR^{A}, —NR^{B}R^{C}, —R^{B}C(O)R^{D}, —C(O)NR^{B}R^{C}, —C(O)R^{D}, —C(O)OR^{D}. In some embodiments, R³ is C₁-C₆-alkyl, C₁-C₆-heteroalkyl, halo, cyano, cycloalkyl, heterocyclyl, aryl, heteroaryl, —OR^{A}, or —C(O)NR^{B}R^{C}. In some embodiments, R³ is C₁-C₆-alkyl. In some embodiments, R³ is C₁-C₆-heteroalkyl. In some embodiments, R³ is halo (e.g., chloro or fluoro). In some embodiments, R³ is cyano. In some embodiments, R³ is cycloalkyl (e.g., cyclopropyl or cyclobutyl). In some embodiments, R³ is heterocyclyl. In some embodiments, R³ is heteroaryl. In some embodiments, R³ is —OR^{A}. In some embodiments, R³ is —C(O)NR^{B}R^{C}.

In some embodiments, R¹ is C₁-C₆-alkyl. In some embodiments, R¹ is CH₃. In some embodiments, A is substituted with 0 or 1 R¹. In some embodiments, B is substituted with 0, 1, or 2 R¹.

In some embodiments, the compound of Formula (I) is a compound of Formula (I-a):

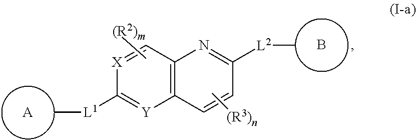

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X and Y are each N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁶ is independently hydrogen, halo, cyano, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; n is 0, 1, or 2; m is 0 or 1; and x is 0, 1, or 2.

In some embodiments, the compound of Formula (I) is a compound of Formula (I-a-i):

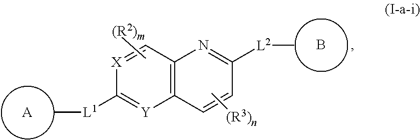

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X and Y are each N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, halo, cyano, oxo, —OR^{A}, —NR^{B}R^{C}, —N^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁶ is independently hydrogen, halo, cyano, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹; each R⁸ is independently C₁-C₆ alkyl, C₁-C₆ heteroalkyl, C₁-C₆ haloalkyl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; n is 0, 1, or 2; m is 0 or 1; and x is 0, 1, or 2.

In some embodiments, the compound of Formula (I) is a compound of Formula (I-b):

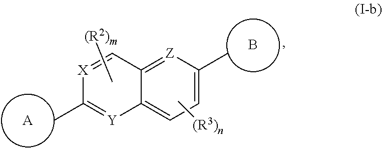

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D} NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. In some embodiments, each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-c):

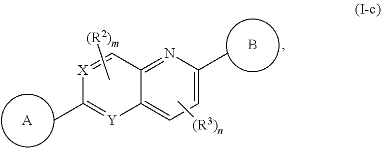

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X, and Y are each N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁—C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-d):

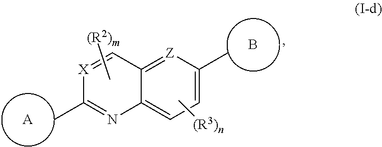

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X and Z are each N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-e):

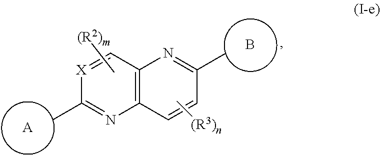

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; X is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)_{X}R^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-f):

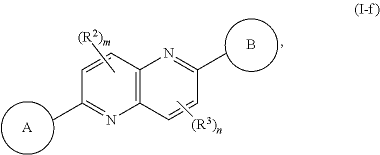

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-g):

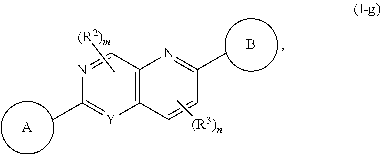

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; Y is N or C(R⁶); each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)_{X}R^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-h):

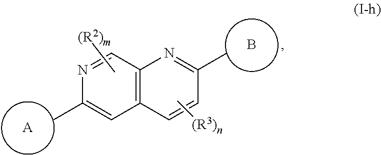

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —R^{B}C(O)R^{D}, —NO₂—C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2. each R² and R³ is independently C₁-C₆-alkyl, C₂-C₆-alkenyl, C₂-C₆-alkynyl, C₁-C₆-heteroalkyl, C₁-C₆-haloalkyl, halo, cyano, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}.

In some embodiments, the compound of Formula (I) is a compound of Formula (I-h-i):

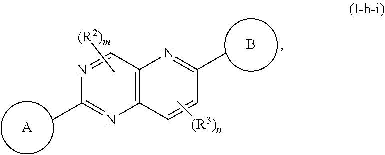

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —R^{B}C(O)R^{D}, —NO₂—C(O)NR^{B}R^{C}, —C(O)R^{D}, —C(O)OR^{D}, or —S(O)ₓR^{D}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

In some embodiments, the compound of Formula (I) is a compound of Formula (I-h-ii):

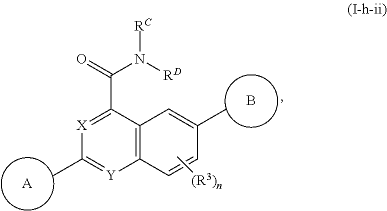

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)_{X}R^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)_{X}R^{D}, wherein each alkyl, alkylene, heteroalkyl, haloalkyl, cycloalkyl, heterocyclyl, aryl, and heteroaryl is optionally substituted with one or more R⁹; each R^{B} and R^{C} is independently hydrogen, C₁-C₆ alkyl, C₁-C₆ heteroalkyl, cycloalkyl, heterocyclyl, or —OR^{A}, wherein each alkyl, alkylene, heteroalkyl, haloalkyl, cycloalkyl, heterocyclyl, aryl, and heteroaryl is optionally substituted with one or more R⁹; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m is 0 or 1; n is 0, 1, or 2; and x is 0, 1, or 2. In an embodiment, when X is CH and Y and Z are each independently N, one of L¹ and L² is independently not N(CH₃) or O. In an embodiment, when X is CH and Y and Z are each independently N, L¹ is not N(CH₃) or O. In an embodiment, when X is CH and Y and Z are each independently N, L² is not N(CH₃) or O. In an embodiment, when X is CH and Y and Z are each independently N, each of L¹ and L² is independently not N(CH₃) or O.

In some embodiments, the compound of Formula (I) is a compound of Formula (I-i):

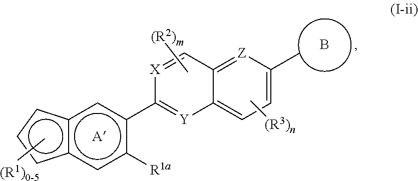

or a pharmaceutically acceptable salt, solvate, hydrate, tautomer, or stereoisomer thereof, wherein A′ is bicyclic heteroaryl or heterocyclyl; B is cycloalkyl, heterocyclyl, aryl, or heteroaryl, each of which is optionally substituted with one or more R¹; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R^{1a} is C₁-C₆-alkyl, C₁-C₆-heteroalkyl, C₁-C₆-haloalkyl, halo, —OR^{A}, —NR^{B}R^{C}, —^{B}C(O)R^{D}, —C(O)NR^{B}R^{C}, —C(O)R^{D}, or —C(O)OR^{D}; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

In some embodiments, the compound of Formula (I) is a compound of Formula (I-j):

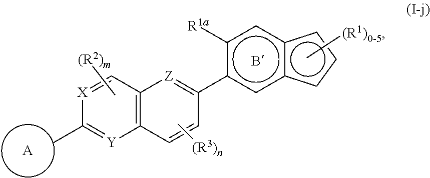

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; B′ is bicyclic heteroaryl or heterocyclyl; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; R^{1a} is C₁-C₆-alkyl, C₁-C₆-heteroalkyl, C₁-C₆-haloalkyl, halo, —OR^{A}, —NR^{B}R^{C}, —^{B}C(O)R^{D}, —C(O)NR^{B}R^{C}, —C(O)R^{D}, or —C(O)OR^{D}; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

In some embodiments, the compound of Formula (I) is a compound of Formula (I-k):

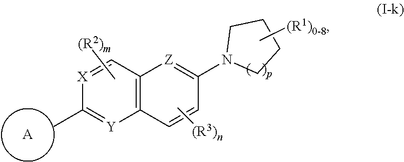

or a pharmaceutically acceptable salt, solvate, hydrate, tautomer, or stereoisomer thereof, wherein A is cycloalkyl, heterocyclyl, aryl, or heteroaryl, each of which is optionally substituted with one or more R¹; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; p is 0, 1, 2, or 3; and x is 0, 1, or 2.

In some embodiments, the compound of Formula (I) is a compound of Formula (I-1):

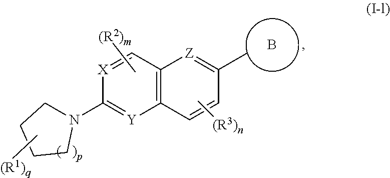

or a pharmaceutically acceptable salt, solvate, hydrate, tautomer, or stereoisomer thereof, wherein B is cycloalkyl, heterocyclyl, aryl, or heteroaryl, each of which is optionally substituted with one or more R¹; X, Y, and Z are each N or C(R⁶), wherein at least one of X, Y, and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C}together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁—C₆-alkyl; m and n are each independently 0, 1, or 2; p is 0, 1, 2, or 3; q is 0, 1, 2, 3, 4, 5, 6, 7, 8, 9, 10, 11, 12, 13, or 14; and x is 0, 1, or 2.

In some embodiments, the compound of Formula (I) is selected from a compound in Table 1, or a pharmaceutically acceptable salt, solvate, hydrate, tautomer, or stereoisomer thereof.

TABLE 1
Exemplary compounds of Formula (I)

| Cmpd | |
|---|---|
| No. | Structure |
| 100 | 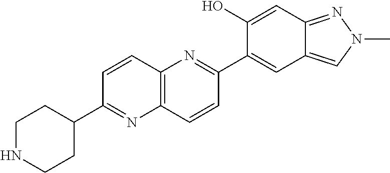 [SMILES: Cn1cc2cc(-c3ccc4nc(C5CCNCC5)ccc4n3)c(O)cc2n1] |
| 101 | 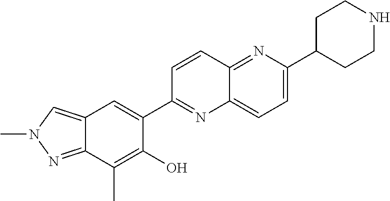 [SMILES: Cc1c(O)c(-c2ccc3nc(C4CCNCC4)ccc3n2)cc2cn(C)nc12] |
| 102 | 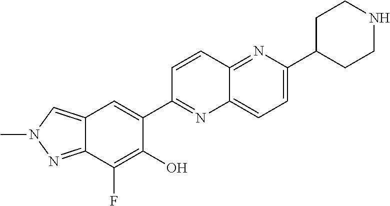 [SMILES: Cn1cc2cc(-c3ccc4nc(C5CCNCC5)ccc4n3)c(O)c(F)c2n1] |
| 103 | 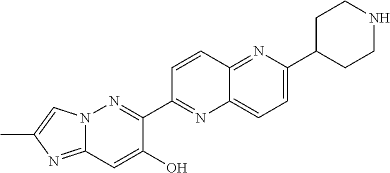 [SMILES: Cc1cn2nc(-c3ccc4nc(C5CCNCC5)ccc4n3)c(O)cc2n1] |
| 104 | 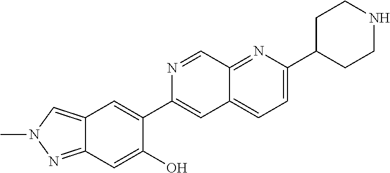 [SMILES: Cn1cc2cc(-c3cc4ccc(C5CCNCC5)nc4cn3)c(O)cc2n1] |
| 105 | 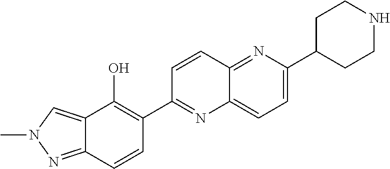 [SMILES: Cn1cc2c(O)c(-c3ccc4nc(C5CCNCC5)ccc4n3)ccc2n1] |
| 106 | 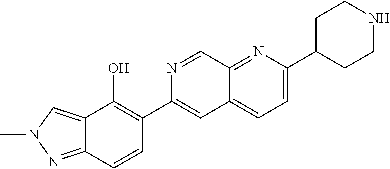 [SMILES: Cn1cc2c(O)c(-c3cc4ccc(C5CCNCC5)nc4cn3)ccc2n1] |
| 107 | 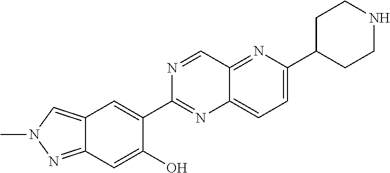 [SMILES: Cn1cc2cc(-c3ncc4nc(C5CCNCC5)ccc4n3)c(O)cc2n1] |
| 108 | 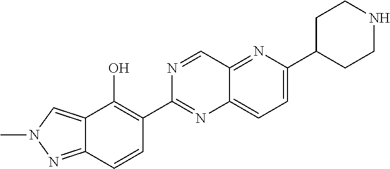 [SMILES: Cn1cc2c(O)c(-c3ncc4nc(C5CCNCC5)ccc4n3)ccc2n1] |
| 109 | 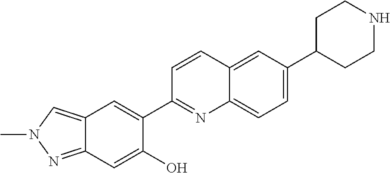 [SMILES: Cn1cc2cc(-c3ccc4cc(C5CCNCC5)ccc4n3)c(O)cc2n1] |
| 113 | 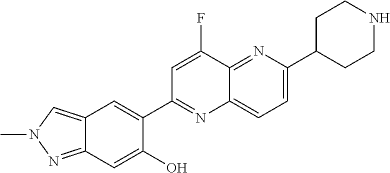 [SMILES: Cn1cc2cc(-c3cc(F)c4nc(C5CCNCC5)ccc4n3)c(O)cc2n1] |
| 114 | 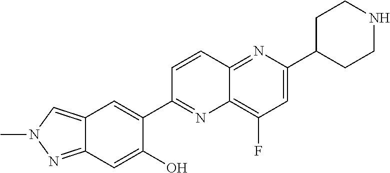 [SMILES: Cn1cc2cc(-c3ccc4nc(C5CCNCC5)cc(F)c4n3)c(O)cc2n1] |
| 115 | 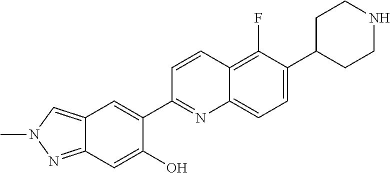 [SMILES: Cn1cc2cc(-c3ccc4c(F)c(C5CCNCC5)ccc4n3)c(O)cc2n1] |
| 116 | 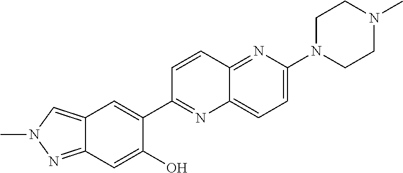 |
| 117 | 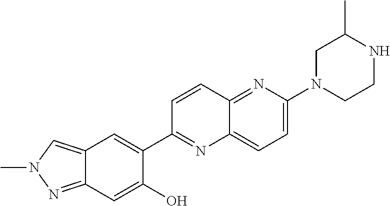 [SMILES: CC1CN(c2ccc3nc(-c4cc5cn(C)nc5cc4O)ccc3n2)CCN1] |
| 118 | 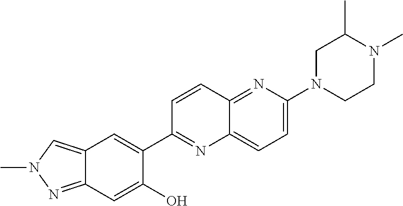 |
| 119 | 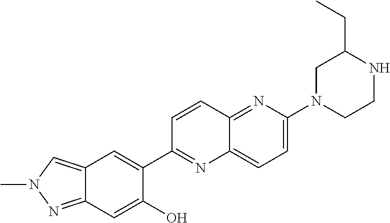 [SMILES: CCC1CN(c2ccc3nc(-c4cc5cn(C)nc5cc4O)ccc3n2)CCN1] |
| 120 | 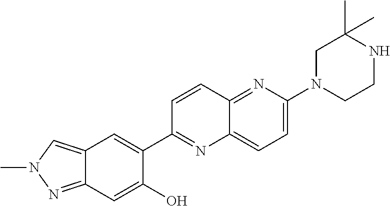 |
| 121 | 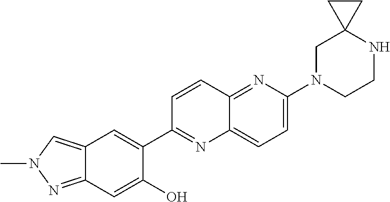 [SMILES: Cn1cc2cc(-c3ccc4nc(N5CCNC6(CC6)C5)ccc4n3)c(O)cc2n1] |
| 122 | 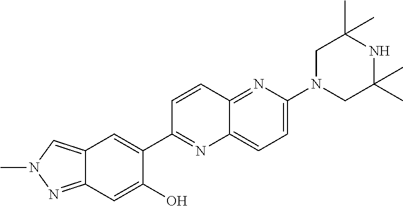 |
| 123 | 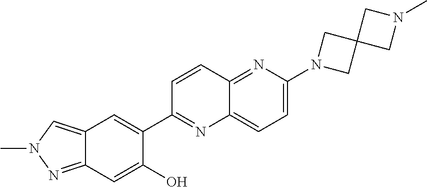 |
| 124 | 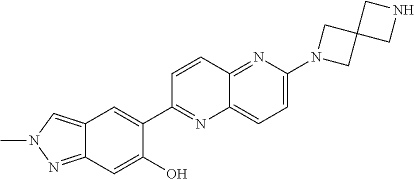 [SMILES: Cn1cc2cc(-c3ccc4nc(N5CC6(CNC6)C5)ccc4n3)c(O)cc2n1] |
| 125 | 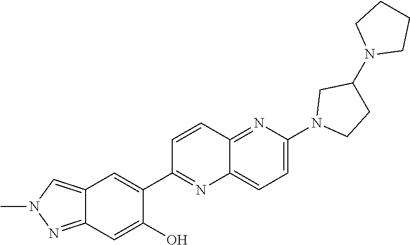 [SMILES: Cn1cc2cc(-c3ccc4nc(N5CCC(N6CCCC6)C5)ccc4n3)c(O)cc2n1] |
| 126 | 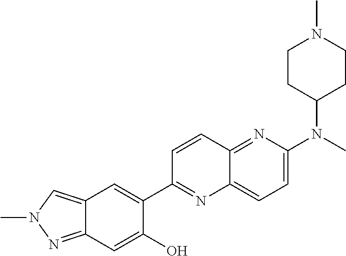 [SMILES: CN1CCC(N(C)c2ccc3nc(-c4cc5cn(C)nc5cc4O)ccc3n2)CC1] |
| 127 | 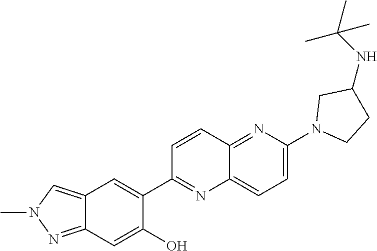 [SMILES: Cn1cc2cc(-c3ccc4nc(N5CCC(NC(C)(C)C)C5)ccc4n3)c(O)cc2n1] |
| 129 | 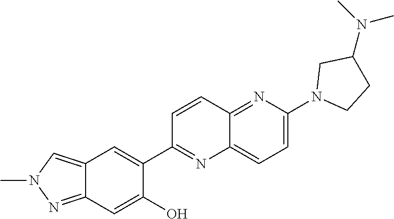 |
| 130 | 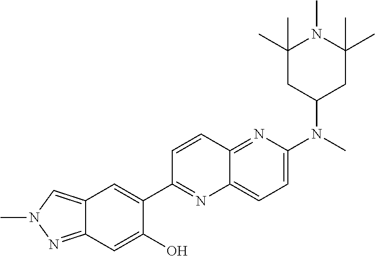 |
| 131 | 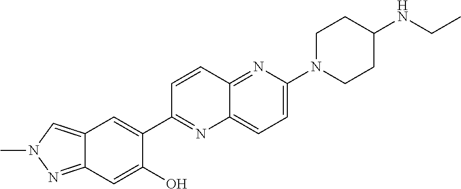 [SMILES: CCNC1CCN(c2ccc3nc(-c4cc5cn(C)nc5cc4O)ccc3n2)CC1] |
| 132 | 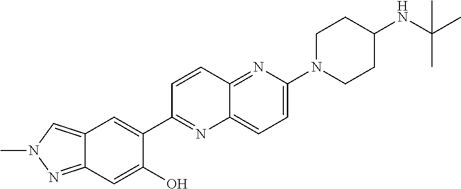 [SMILES: Cn1cc2cc(-c3ccc4nc(N5CCC(NC(C)(C)C)CC5)ccc4n3)c(O)cc2n1] |
| 133 | 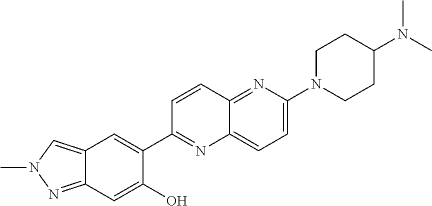 |
| 134 | 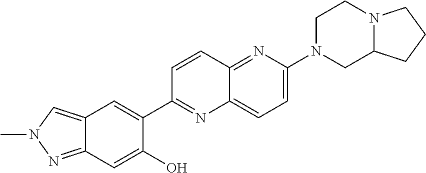 [SMILES: Cn1cc2cc(-c3ccc4nc(N5CCN6CCCC6C5)ccc4n3)c(O)cc2n1] |
| 135 | 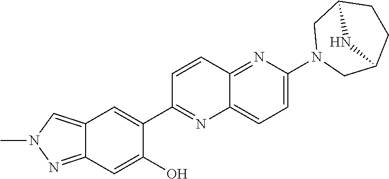 [SMILES: Cn1cc2cc(-c3ccc4nc(N5C[C@H]6CC[C@@H](C5)N6)ccc4n3)c(O)cc2n1] |
| 136 | 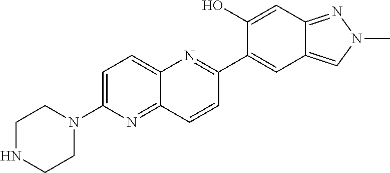 [SMILES: Cn1cc2cc(-c3ccc4nc(N5CCNCC5)ccc4n3)c(O)cc2n1] |
| 137 | 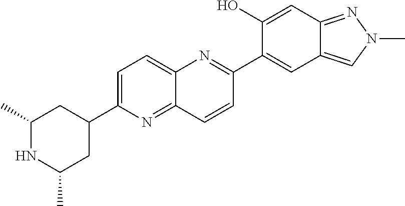 |
| 138 | 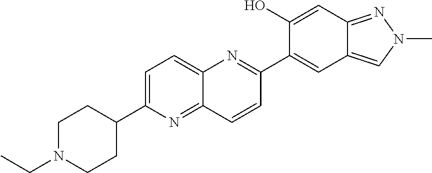 [SMILES: CCN1CCC(c2ccc3nc(-c4cc5cn(C)nc5cc4O)ccc3n2)CC1] |
| 139 | 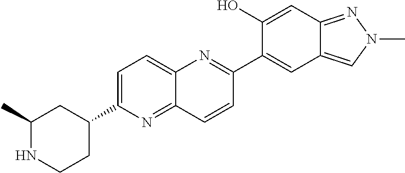 |
| 140 | 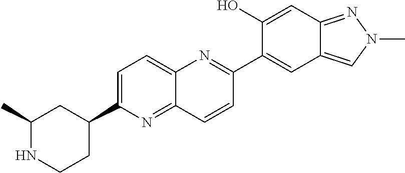 |
| 141 | 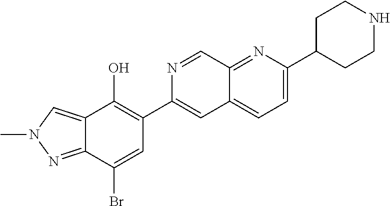 [SMILES: Cn1cc2c(O)c(-c3cc4ccc(C5CCNCC5)nc4cn3)cc(Br)c2n1] |
| 142 | 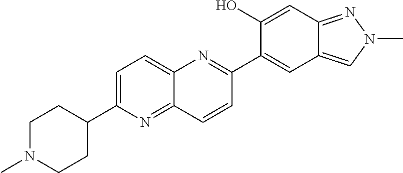 |
| 143 | 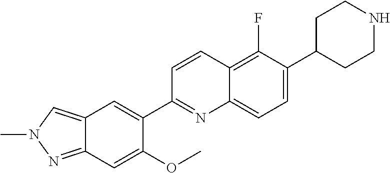 [SMILES: COc1cc2nn(C)cc2cc1-c1ccc2c(F)c(C3CCNCC3)ccc2n1] |
| 144 | 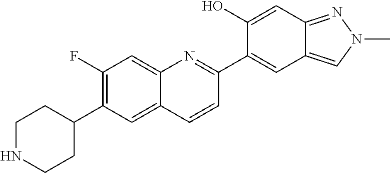 [SMILES: Cn1cc2cc(-c3ccc4cc(C5CCNCC5)c(F)cc4n3)c(O)cc2n1] |
| 145 | 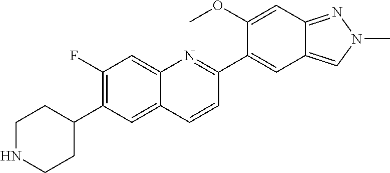 [SMILES: COc1cc2nn(C)cc2cc1-c1ccc2cc(C3CCNCC3)c(F)cc2n1] |
| 146 | 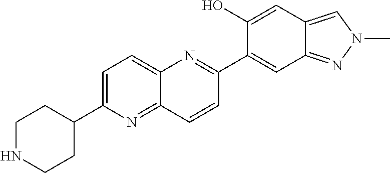 [SMILES: Cn1cc2cc(O)c(-c3ccc4nc(C5CCNCC5)ccc4n3)cc2n1] |
| 147 | 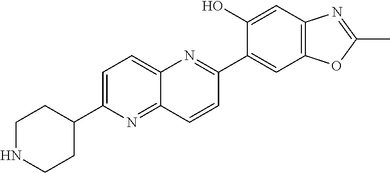 [SMILES: Cc1nc2cc(O)c(-c3ccc4nc(C5CCNCC5)ccc4n3)cc2o1] |
| 148 | 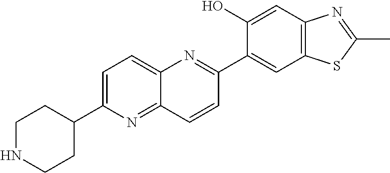 [SMILES: Cc1nc2cc(O)c(-c3ccc4nc(C5CCNCC5)ccc4n3)cc2s1] |
| 149 | 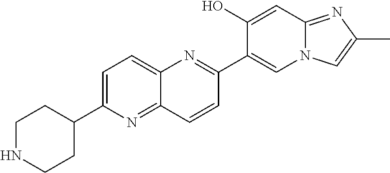 [SMILES: Cc1cn2cc(-c3ccc4nc(C5CCNCC5)ccc4n3)c(O)cc2n1] |
| 150 | 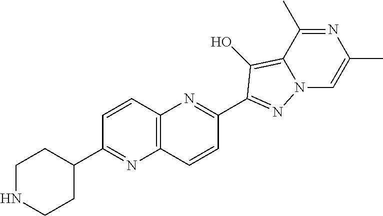 [SMILES: Cc1cn2nc(-c3ccc4nc(C5CCNCC5)ccc4n3)c(O)c2c(C)n1] |
| 151 | 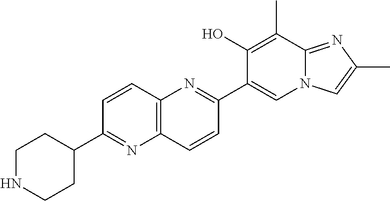 [SMILES: Cc1cn2cc(-c3ccc4nc(C5CCNCC5)ccc4n3)c(O)c(C)c2n1] |
| 152 | 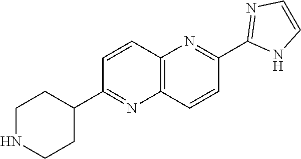 [SMILES: c1c[nH]c(-c2ccc3nc(C4CCNCC4)ccc3n2)n1] |
| 153 | 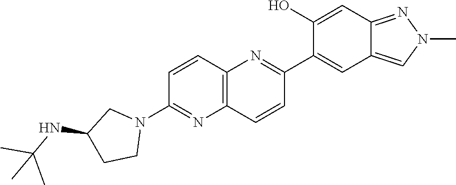 [SMILES: Cn1cc2cc(-c3ccc4nc(N5CC[C@@H](NC(C)(C)C)C5)ccc4n3)c(O)cc2n1] |
| 154 | 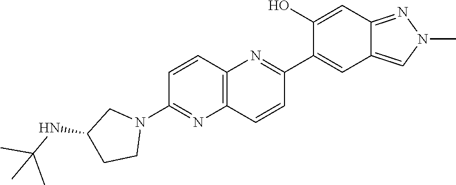 [SMILES: Cn1cc2cc(-c3ccc4nc(N5CC[C@H](NC(C)(C)C)C5)ccc4n3)c(O)cc2n1] |
| 155 | 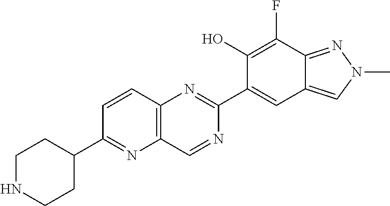 [SMILES: Cn1cc2cc(-c3ncc4nc(C5CCNCC5)ccc4n3)c(O)c(F)c2n1] |
| 156 | 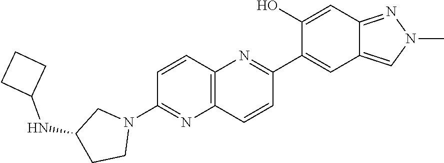 [SMILES: Cn1cc2cc(-c3ccc4nc(N5CC[C@H](NC6CCC6)C5)ccc4n3)c(O)cc2n1] |
| 157 | 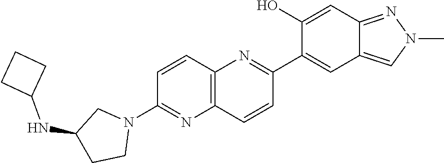 [SMILES: Cn1cc2cc(-c3ccc4nc(N5CC[C@@H](NC6CCC6)C5)ccc4n3)c(O)cc2n1] |
| 158 | 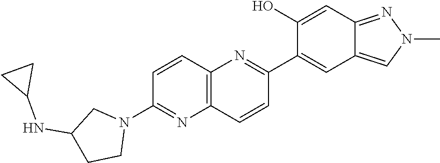 [SMILES: Cn1cc2cc(-c3ccc4nc(N5CCC(NC6CC6)C5)ccc4n3)c(O)cc2n1] |
| 159 | 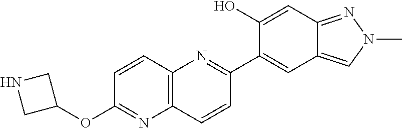 [SMILES: Cn1cc2cc(-c3ccc4nc(OC5CNC5)ccc4n3)c(O)cc2n1] |
| 160 | 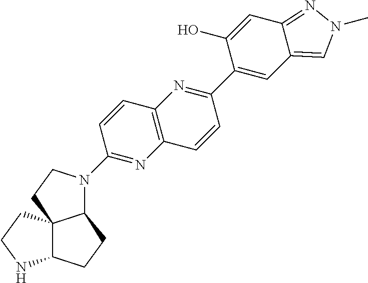 [SMILES: Cn1cc2cc(-c3ccc4nc(N5CC[C@]67CCN[C@H]6CC[C@H]57)ccc4n3)c(O)cc2n1] |
| 161 | 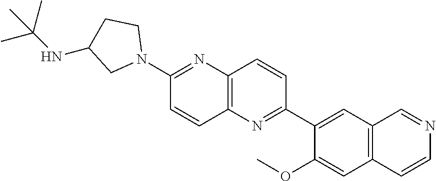 [SMILES: COc1cc2ccncc2cc1-c1ccc2nc(N3CCC(NC(C)(C)C)C3)ccc2n1] |
| 162 | 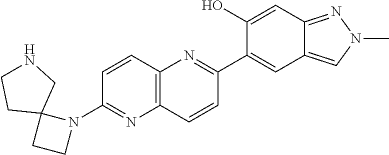 [SMILES: Cn1cc2cc(-c3ccc4nc(N5CCC56CCNC6)ccc4n3)c(O)cc2n1] |
| 163 | 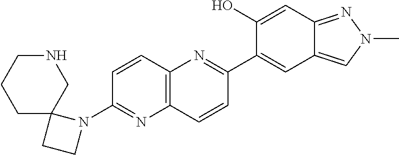 [SMILES: Cn1cc2cc(-c3ccc4nc(N5CCC56CCCNC6)ccc4n3)c(O)cc2n1] |
| 164 | 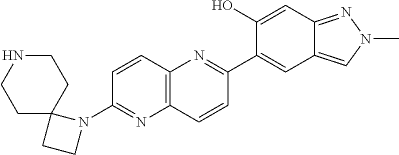 [SMILES: Cn1cc2cc(-c3ccc4nc(N5CCC56CCNCC6)ccc4n3)c(O)cc2n1] |
| 165 | 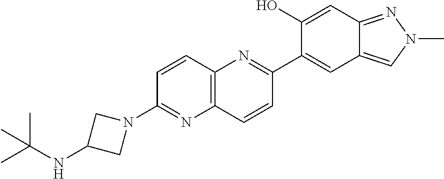 [SMILES: Cn1cc2cc(-c3ccc4nc(N5CC(NC(C)(C)C)C5)ccc4n3)c(O)cc2n1] |
| 166 | 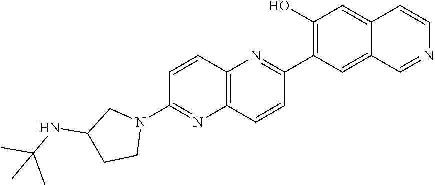 [SMILES: CC(C)(C)NC1CCN(c2ccc3nc(-c4cc5cnccc5cc4O)ccc3n2)C1] |
| 167 | 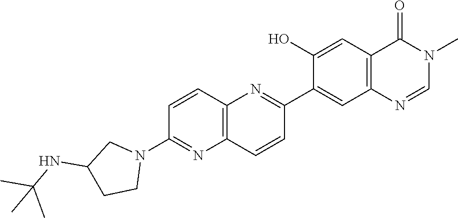 [SMILES: Cn1cnc2cc(-c3ccc4nc(N5CCC(NC(C)(C)C)C5)ccc4n3)c(O)cc2c1=O] |
| 168 | 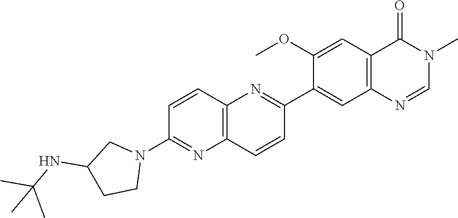 [SMILES: COc1cc2c(=O)n(C)cnc2cc1-c1ccc2nc(N3CCC(NC(C)(C)C)C3)ccc2n1] |
| 169 | 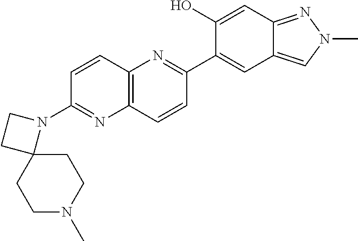 [SMILES: CN1CCC2(CC1)CCN2c1ccc2nc(-c3cc4cn(C)nc4cc3O)ccc2n1] |
| 170 | 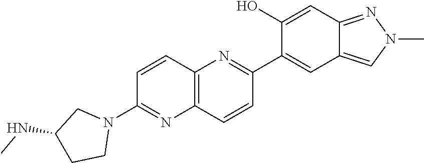 |
| 171 | 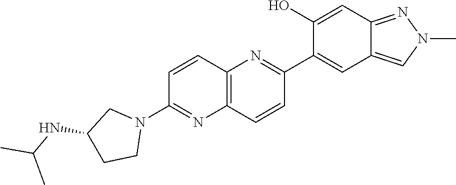 [SMILES: CC(C)N[C@H]1CCN(c2ccc3nc(-c4cc5cn(C)nc5cc4O)ccc3n2)C1] |
| 172 | 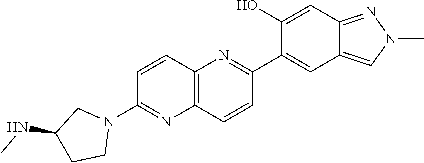 |
| 173 | 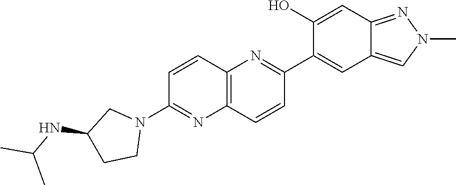 [SMILES: CC(C)N[C@@H]1CCN(c2ccc3nc(-c4cc5cn(C)nc5cc4O)ccc3n2)C1] |
| 174 | 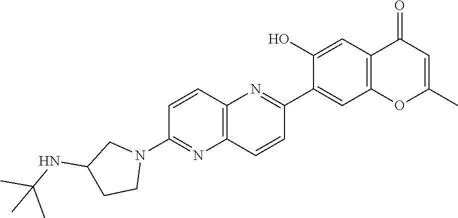 [SMILES: Cc1cc(=O)c2cc(O)c(-c3ccc4nc(N5CCC(NC(C)(C)C)C5)ccc4n3)cc2o1] |
| 175 | 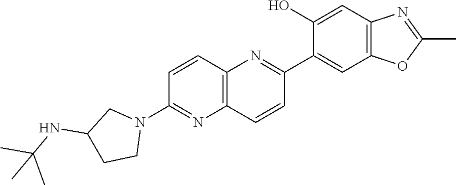 [SMILES: Cc1nc2cc(O)c(-c3ccc4nc(N5CCC(NC(C)(C)C)C5)ccc4n3)cc2o1] |
| 176 | 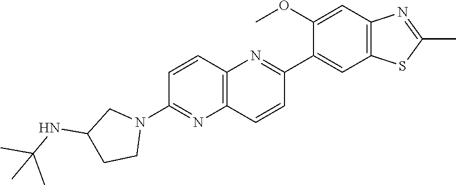 [SMILES: COc1cc2nc(C)sc2cc1-c1ccc2nc(N3CCC(NC(C)(C)C)C3)ccc2n1] |
| 177 | 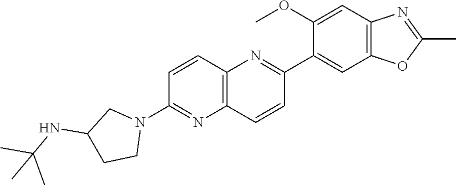 [SMILES: COc1cc2nc(C)oc2cc1-c1ccc2nc(N3CCC(NC(C)(C)C)C3)ccc2n1] |
| 178 | 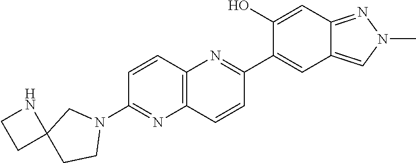 [SMILES: Cn1cc2cc(-c3ccc4nc(N5CCC6(CCN6)C5)ccc4n3)c(O)cc2n1] |
| 179 | 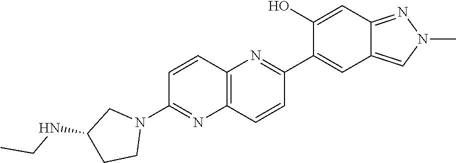 [SMILES: CCN[C@H]1CCN(c2ccc3nc(-c4cc5cn(C)nc5cc4O)ccc3n2)C1] |
| 180 | 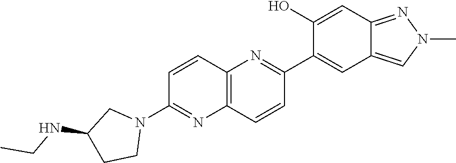 [SMILES: CCN[C@@H]1CCN(c2ccc3nc(-c4cc5cn(C)nc5cc4O)ccc3n2)C1] |
| 181 | 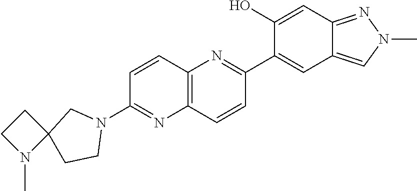 [SMILES: CN1CCC12CCN(c1ccc3nc(-c4cc5cn(C)nc5cc4O)ccc3n1)C2] |
| 182 | 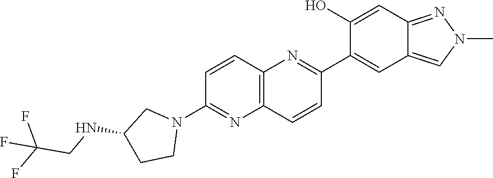 [SMILES: Cn1cc2cc(-c3ccc4nc(N5CC[C@H](NCC(F)(F)F)C5)ccc4n3)c(O)cc2n1] |
| 183 | 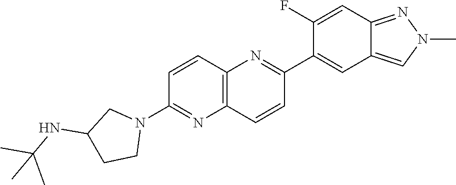 [SMILES: Cn1cc2cc(-c3ccc4nc(N5CCC(NC(C)(C)C)C5)ccc4n3)c(F)cc2n1] |
| 184 | 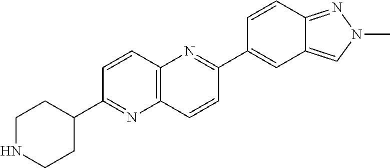 [SMILES: Cn1cc2cc(-c3ccc4nc(C5CCNCC5)ccc4n3)ccc2n1] |
| 185 | 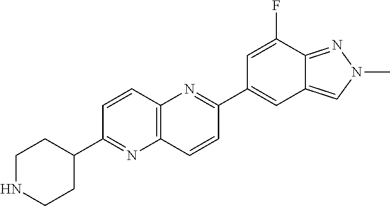 [SMILES: Cn1cc2cc(-c3ccc4nc(C5CCNCC5)ccc4n3)cc(F)c2n1] |
| 186 | 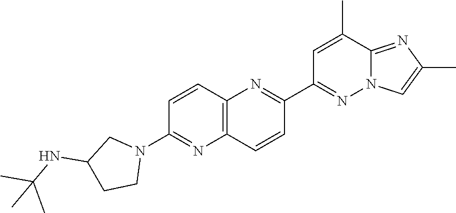 [SMILES: Cc1cn2nc(-c3ccc4nc(N5CCC(NC(C)(C)C)C5)ccc4n3)cc(C)c2n1] |
| 187 | 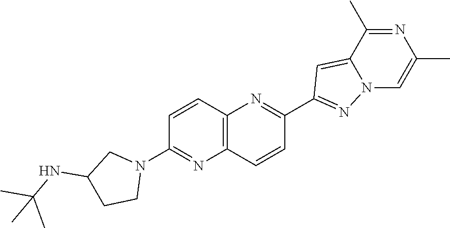 [SMILES: Cc1cn2nc(-c3ccc4nc(N5CCC(NC(C)(C)C)C5)ccc4n3)cc2c(C)n1] |
| 188 | 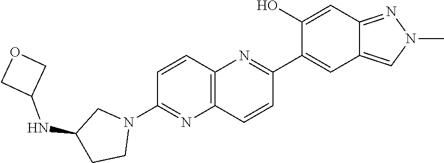 [SMILES: Cn1cc2cc(-c3ccc4nc(N5CC[C@@H](NC6COC6)C5)ccc4n3)c(O)cc2n1] |
| 189 | 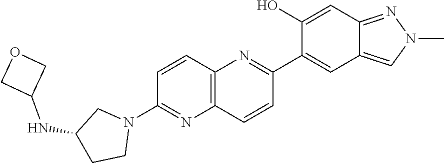 [SMILES: Cn1cc2cc(-c3ccc4nc(N5CC[C@H](NC6COC6)C5)ccc4n3)c(O)cc2n1] |
| 190 | 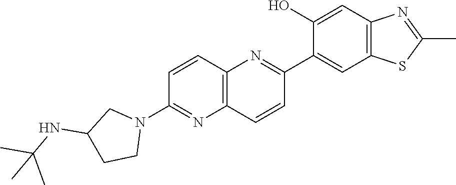 [SMILES: Cc1nc2cc(O)c(-c3ccc4nc(N5CCC(NC(C)(C)C)C5)ccc4n3)cc2s1] |
| 191 | 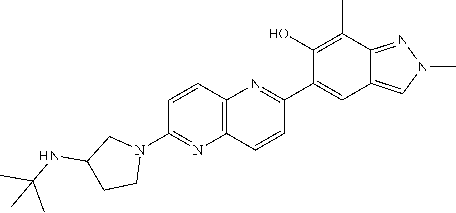 [SMILES: Cc1c(O)c(-c2ccc3nc(N4CCC(NC(C)(C)C)C4)ccc3n2)cc2cn(C)nc12] |
| 192 | 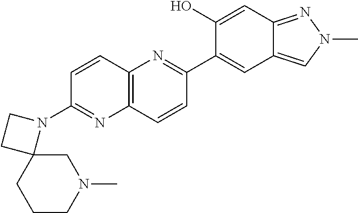 [SMILES: CN1CCCC2(CCN2c2ccc3nc(-c4cc5cn(C)nc5cc4O)ccc3n2)C1] |
| 193 | 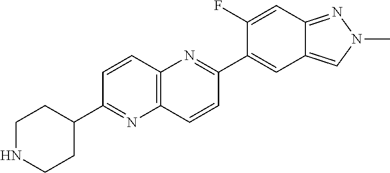 [SMILES: Cn1cc2cc(-c3ccc4nc(C5CCNCC5)ccc4n3)c(F)cc2n1] |
| 194 | 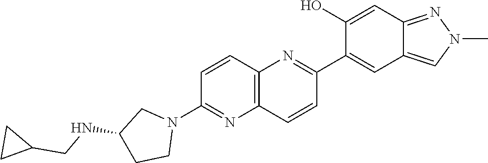 [SMILES: Cn1cc2cc(-c3ccc4nc(N5CC[C@H](NCC6CC6)C5)ccc4n3)c(O)cc2n1] |
| 195 | 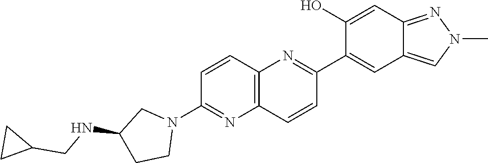 [SMILES: Cn1cc2cc(-c3ccc4nc(N5CC[C@@H](NCC6CC6)C5)ccc4n3)c(O)cc2n1] |
| 196 | 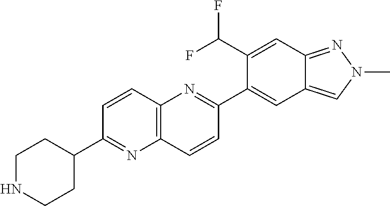 [SMILES: Cn1cc2cc(-c3ccc4nc(C5CCNCC5)ccc4n3)c(C(F)F)cc2n1] |
| 197 | 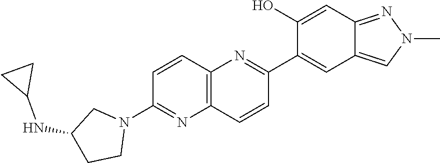 [SMILES: Cn1cc2cc(-c3ccc4nc(N5CC[C@H](NC6CC6)C5)ccc4n3)c(O)cc2n1] |
| 198 | 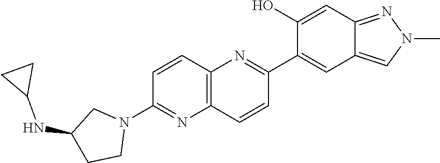 [SMILES: Cn1cc2cc(-c3ccc4nc(N5CC[C@@H](NC6CC6)C5)ccc4n3)c(O)cc2n1] |
| 199 | 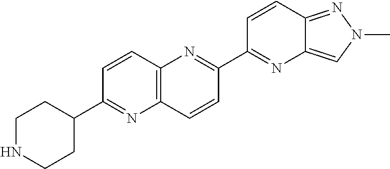 [SMILES: Cn1cc2nc(-c3ccc4nc(C5CCNCC5)ccc4n3)ccc2n1] |
| 200 | 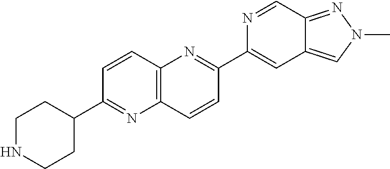 [SMILES: Cn1cc2cc(-c3ccc4nc(C5CCNCC5)ccc4n3)ncc2n1] |
| 201 | 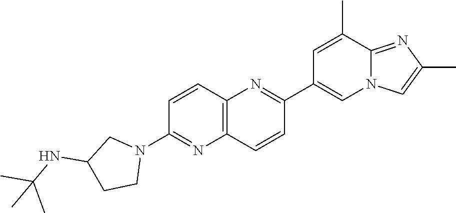 [SMILES: Cc1cn2cc(-c3ccc4nc(N5CCC(NC(C)(C)C)C5)ccc4n3)cc(C)c2n1] |
| 202 | 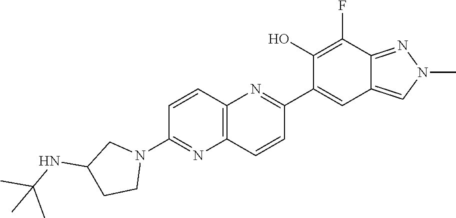 [SMILES: Cn1cc2cc(-c3ccc4nc(N5CCC(NC(C)(C)C)C5)ccc4n3)c(O)c(F)c2n1] |
| 203 | 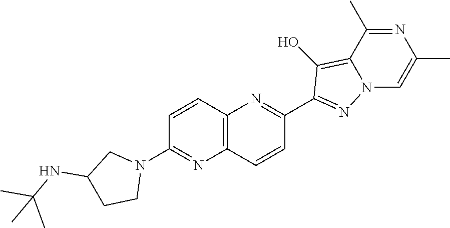 [SMILES: Cc1cn2nc(-c3ccc4nc(N5CCC(NC(C)(C)C)C5)ccc4n3)c(O)c2c(C)n1] |
| 204 | 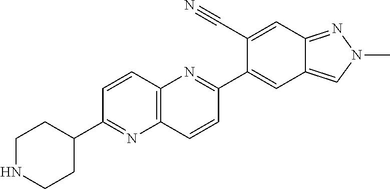 [SMILES: Cn1cc2cc(-c3ccc4nc(C5CCNCC5)ccc4n3)c(C#N)cc2n1] |
| 205 | 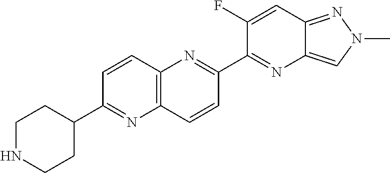 [SMILES: Cn1cc2nc(-c3ccc4nc(C5CCNCC5)ccc4n3)c(F)cc2n1] |
| 206 | 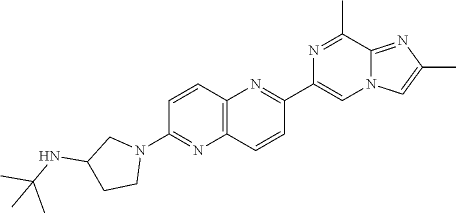 [SMILES: Cc1cn2cc(-c3ccc4nc(N5CCC(NC(C)(C)C)C5)ccc4n3)nc(C)c2n1] |
| 207 | 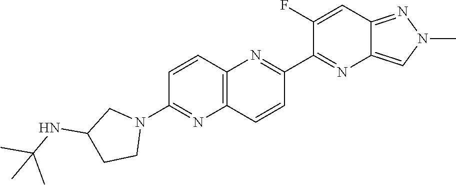 [SMILES: Cn1cc2nc(-c3ccc4nc(N5CCC(NC(C)(C)C)C5)ccc4n3)c(F)cc2n1] |
| 208 | 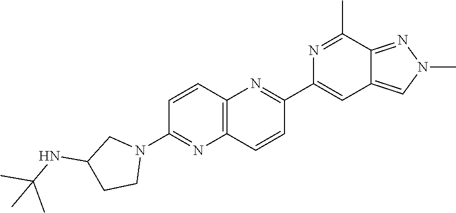 [SMILES: Cc1nc(-c2ccc3nc(N4CCC(NC(C)(C)C)C4)ccc3n2)cc2cn(C)nc12] |
| 209 | 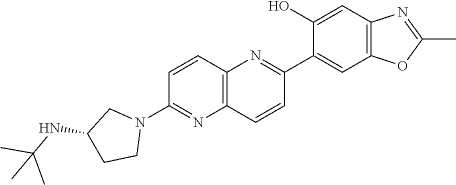 [SMILES: Cc1nc2cc(O)c(-c3ccc4nc(N5CC[C@H](NC(C)(C)C)C5)ccc4n3)cc2o1] |
| 210 | 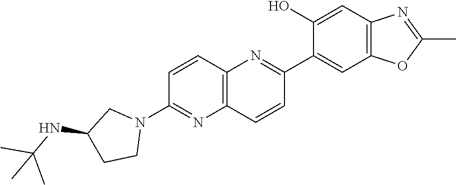 [SMILES: Cc1nc2cc(O)c(-c3ccc4nc(N5CC[C@@H](NC(C)(C)C)C5)ccc4n3)cc2o1] |
| 211 | 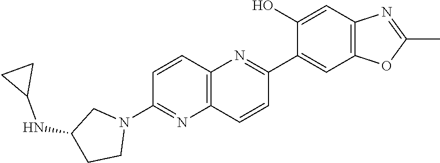 [SMILES: Cc1nc2cc(O)c(-c3ccc4nc(N5CC[C@H](NC6CC6)C5)ccc4n3)cc2o1] |
| 212 | 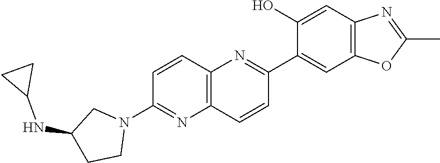 [SMILES: Cc1nc2cc(O)c(-c3ccc4nc(N5CC[C@@H](NC6CC6)C5)ccc4n3)cc2o1] |
| 213 | 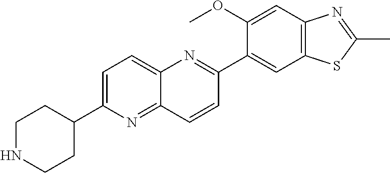 [SMILES: COc1cc2nc(C)sc2cc1-c1ccc2nc(C3CCNCC3)ccc2n1] |
| 214 | 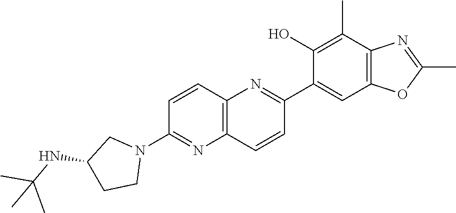 [SMILES: Cc1nc2c(C)c(O)c(-c3ccc4nc(N5CC[C@H](NC(C)(C)C)C5)ccc4n3)cc2o1] |
| 215 | 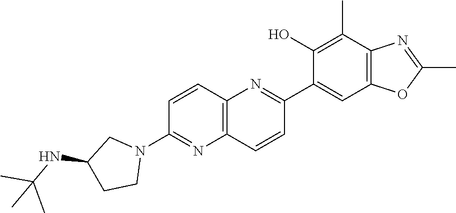 [SMILES: Cc1nc2c(C)c(O)c(-c3ccc4nc(N5CC[C@@H](NC(C)(C)C)C5)ccc4n3)cc2o1] |
| 216 | 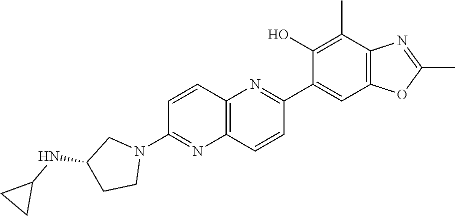 [SMILES: Cc1nc2c(C)c(O)c(-c3ccc4nc(N5CC[C@H](NC6CC6)C5)ccc4n3)cc2o1] |
| 217 | 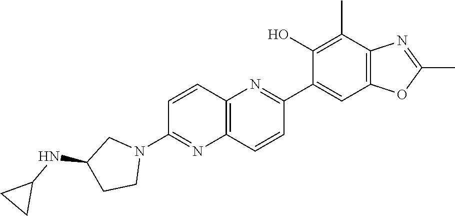 [SMILES: Cc1nc2c(C)c(O)c(-c3ccc4nc(N5CC[C@@H](NC6CC6)C5)ccc4n3)cc2o1] |
| 218 | 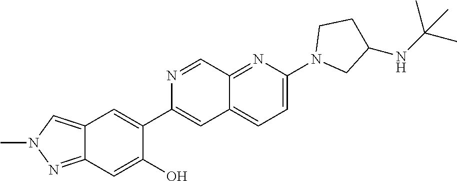 [SMILES: Cn1cc2cc(-c3cc4ccc(N5CCC(NC(C)(C)C)C5)nc4cn3)c(O)cc2n1] |
| 219 | 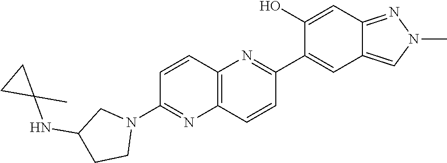 [SMILES: Cn1cc2cc(-c3ccc4nc(N5CCC(NC6(C)CC6)C5)ccc4n3)c(O)cc2n1] |
| 220 | 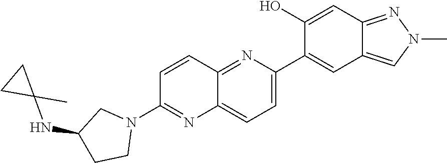 [SMILES: Cn1cc2cc(-c3ccc4nc(N5CC[C@@H](NC6(C)CC6)C5)ccc4n3)c(O)cc2n1] |
| 221 | 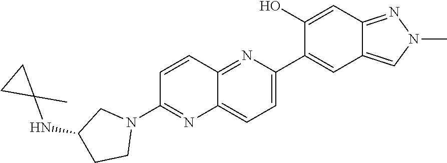 [SMILES: Cn1cc2cc(-c3ccc4nc(N5CC[C@H](NC6(C)CC6)C5)ccc4n3)c(O)cc2n1] |
| 222 | 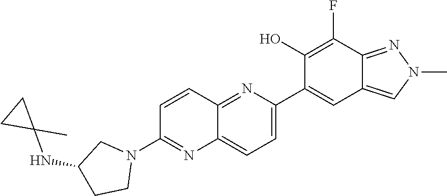 [SMILES: Cn1cc2cc(-c3ccc4nc(N5CC[C@H](NC6(C)CC6)C5)ccc4n3)c(O)c(F)c2n1] |
| 223 | 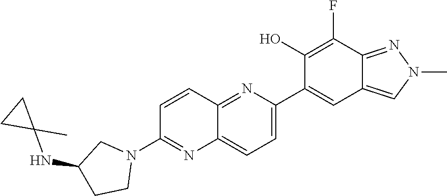 [SMILES: Cn1cc2cc(-c3ccc4nc(N5CC[C@@H](NC6(C)CC6)C5)ccc4n3)c(O)c(F)c2n1] |
| 224 | 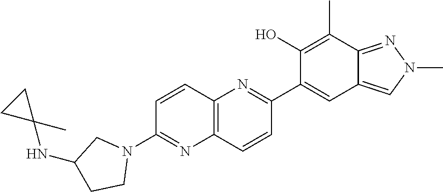 [SMILES: Cc1c(O)c(-c2ccc3nc(N4CCC(NC5(C)CC5)C4)ccc3n2)cc2cn(C)nc12] |
| 225 | 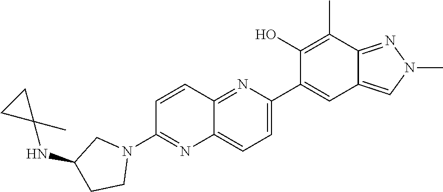 [SMILES: Cc1c(O)c(-c2ccc3nc(N4CC[C@@H](NC5(C)CC5)C4)ccc3n2)cc2cn(C)nc12] |
| 226 | 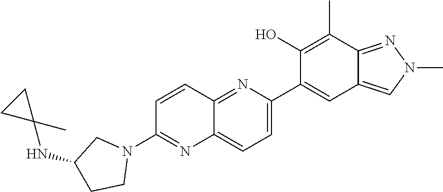 [SMILES: Cc1c(O)c(-c2ccc3nc(N4CC[C@H](NC5(C)CC5)C4)ccc3n2)cc2cn(C)nc12] |
| 228 | 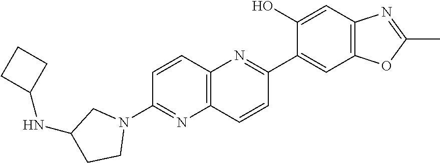 [SMILES: Cc1nc2cc(O)c(-c3ccc4nc(N5CCC(NC6CCC6)C5)ccc4n3)cc2o1] |
| 229 | 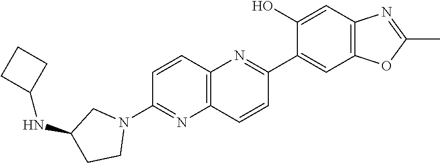 [SMILES: Cc1nc2cc(O)c(-c3ccc4nc(N5CC[C@@H](NC6CCC6)C5)ccc4n3)cc2o1] |
| 230 | 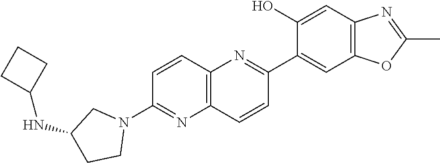 [SMILES: Cc1nc2cc(O)c(-c3ccc4nc(N5CC[C@H](NC6CCC6)C5)ccc4n3)cc2o1] |
| 231 | 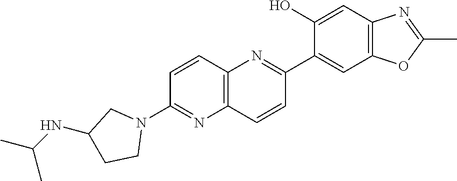 [SMILES: Cc1nc2cc(O)c(-c3ccc4nc(N5CCC(NC(C)C)C5)ccc4n3)cc2o1] |
| 232 | 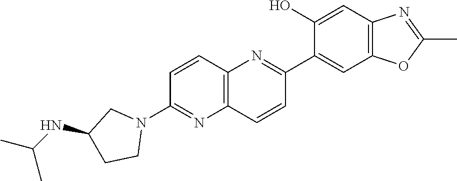 [SMILES: Cc1nc2cc(O)c(-c3ccc4nc(N5CC[C@@H](NC(C)C)C5)ccc4n3)cc2o1] |
| 233 | 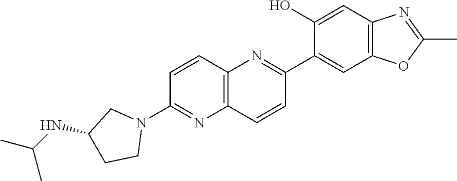 [SMILES: Cc1nc2cc(O)c(-c3ccc4nc(N5CC[C@H](NC(C)C)C5)ccc4n3)cc2o1] |
| 234 | 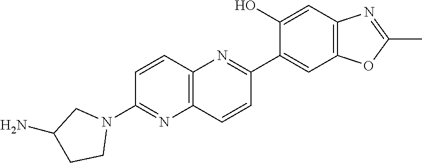 [SMILES: Cc1nc2cc(O)c(-c3ccc4nc(N5CCC(N)C5)ccc4n3)cc2o1] |
| 235 | 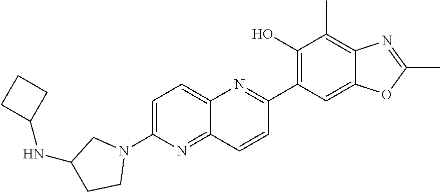 [SMILES: Cc1nc2c(C)c(O)c(-c3ccc4nc(N5CCC(NC6CCC6)C5)ccc4n3)cc2o1] |
| 236 | 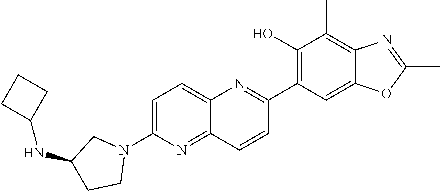 [SMILES: Cc1nc2c(C)c(O)c(-c3ccc4nc(N5CC[C@@H](NC6CCC6)C5)ccc4n3)cc2o1] |
| 237 | 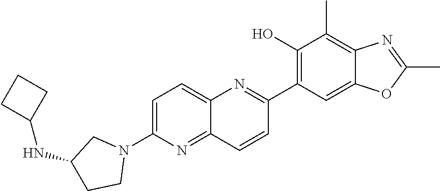 [SMILES: Cc1nc2c(C)c(O)c(-c3ccc4nc(N5CC[C@H](NC6CCC6)C5)ccc4n3)cc2o1] |
| 238 | 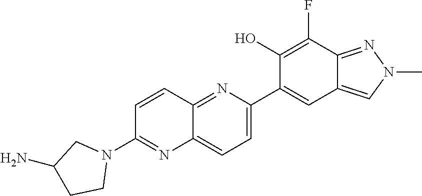 [SMILES: Cn1cc2cc(-c3ccc4nc(N5CCC(N)C5)ccc4n3)c(O)c(F)c2n1] |
| 241 | 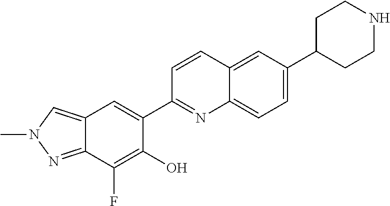 [SMILES: Cn1cc2cc(-c3ccc4cc(C5CCNCC5)ccc4n3)c(O)c(F)c2n1] |
| 245 | 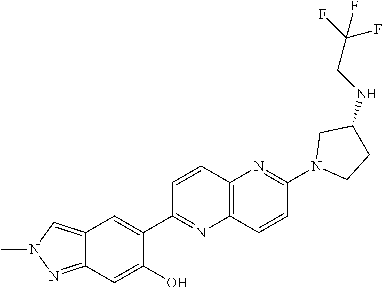 [SMILES: Cn1cc2cc(-c3ccc4nc(N5CC[C@@H](NCC(F)(F)F)C5)ccc4n3)c(O)cc2n1] |
| 246 | 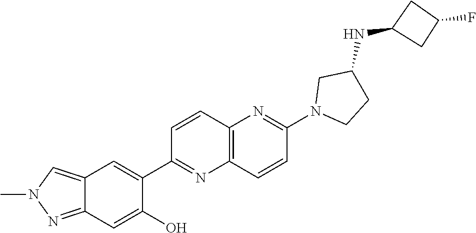 [SMILES: Cn1cc2cc(-c3ccc4nc(N5CC[C@@H](N[C@H]6C[C@H](F)C6)C5)ccc4n3)c(O)cc2n1] |
| 247 | 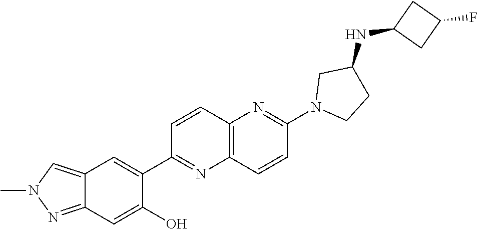 [SMILES: Cn1cc2cc(-c3ccc4nc(N5CC[C@H](N[C@H]6C[C@H](F)C6)C5)ccc4n3)c(O)cc2n1] |
| 248 | 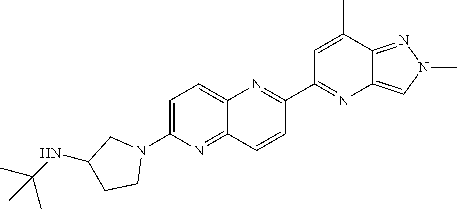 [SMILES: Cc1cc(-c2ccc3nc(N4CCC(NC(C)(C)C)C4)ccc3n2)nc2cn(C)nc12] |
| 249 | 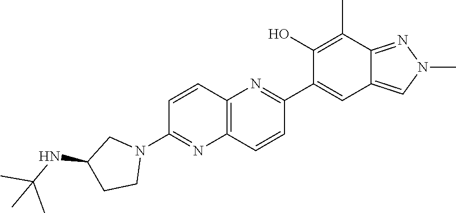 [SMILES: Cc1c(O)c(-c2ccc3nc(N4CC[C@@H](NC(C)(C)C)C4)ccc3n2)cc2cn(C)nc12] |
| 250 | 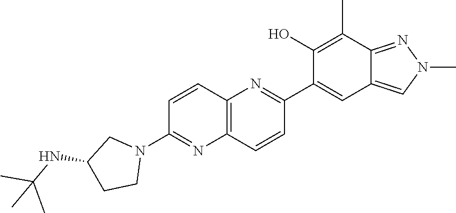 [SMILES: Cc1c(O)c(-c2ccc3nc(N4CC[C@H](NC(C)(C)C)C4)ccc3n2)cc2cn(C)nc12] |
| 251 | 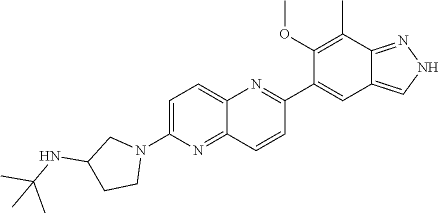 [SMILES: COc1c(-c2ccc3nc(N4CCC(NC(C)(C)C)C4)ccc3n2)cc2c[nH]nc2c1C] |
| 252 | 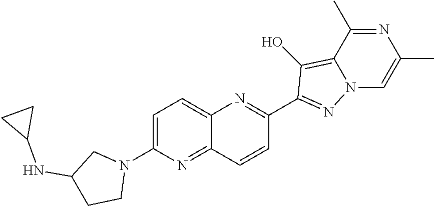 [SMILES: Cc1cn2nc(-c3ccc4nc(N5CCC(NC6CC6)C5)ccc4n3)c(O)c2c(C)n1] |
| 253 | 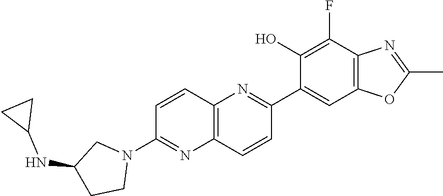 [SMILES: Cc1nc2c(F)c(O)c(-c3ccc4nc(N5CC[C@@H](NC6CC6)C5)ccc4n3)cc2o1] |
| 254 | 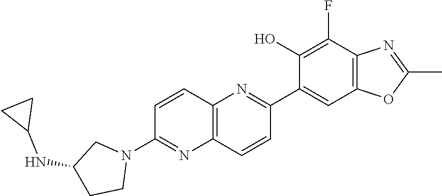 [SMILES: Cc1nc2c(F)c(O)c(-c3ccc4nc(N5CC[C@H](NC6CC6)C5)ccc4n3)cc2o1] |
| 255 | 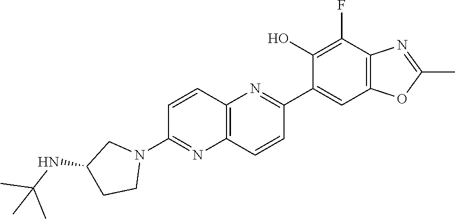 [SMILES: Cc1nc2c(F)c(O)c(-c3ccc4nc(N5CC[C@H](NC(C)(C)C)C5)ccc4n3)cc2o1] |
| 256 | 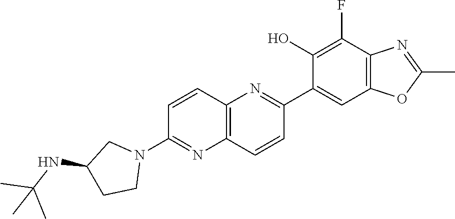 [SMILES: Cc1nc2c(F)c(O)c(-c3ccc4nc(N5CC[C@@H](NC(C)(C)C)C5)ccc4n3)cc2o1] |
| 257 | 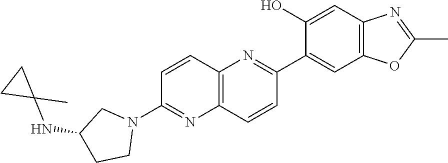 [SMILES: Cc1nc2cc(O)c(-c3ccc4nc(N5CC[C@H](NC6(C)CC6)C5)ccc4n3)cc2o1] |
| 258 | 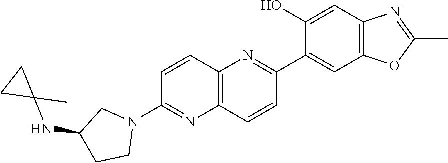 [SMILES: Cc1nc2cc(O)c(-c3ccc4nc(N5CC[C@@H](NC6(C)CC6)C5)ccc4n3)cc2o1] |
| 259 | 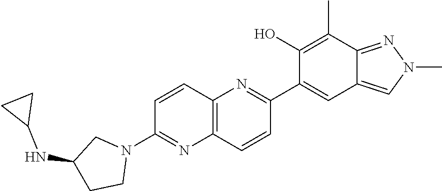 [SMILES: Cc1c(O)c(-c2ccc3nc(N4CC[C@@H](NC5CC5)C4)ccc3n2)cc2cn(C)nc12] |
| 260 | 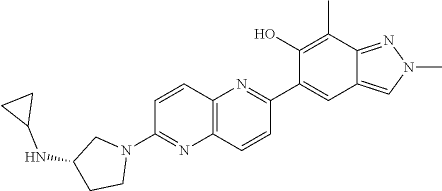 [SMILES: Cc1c(O)c(-c2ccc3nc(N4CC[C@H](NC5CC5)C4)ccc3n2)cc2cn(C)nc12] |
| 262 | 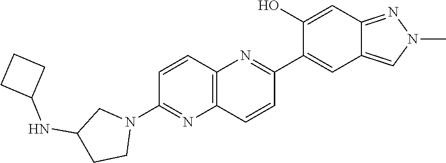 [SMILES: Cn1cc2cc(-c3ccc4nc(N5CCC(NC6CCC6)C5)ccc4n3)c(O)cc2n1] |
| 263 | 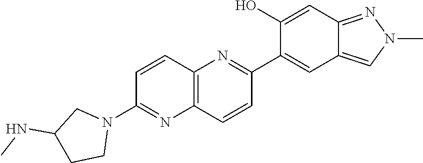 |
| 264 | 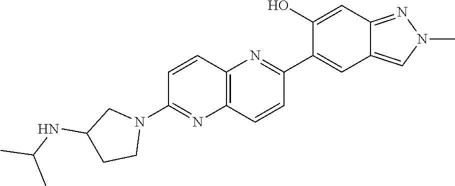 [SMILES: CC(C)NC1CCN(c2ccc3nc(-c4cc5cn(C)nc5cc4O)ccc3n2)C1] |
| 265 | 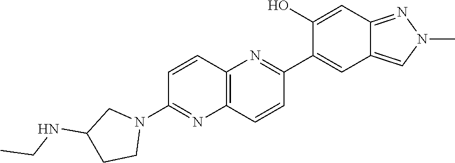 [SMILES: CCNC1CCN(c2ccc3nc(-c4cc5cn(C)nc5cc4O)ccc3n2)C1] |
| 266 | 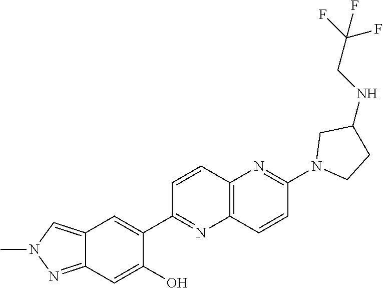 [SMILES: Cn1cc2cc(-c3ccc4nc(N5CCC(NCC(F)(F)F)C5)ccc4n3)c(O)cc2n1] |
| 267 | 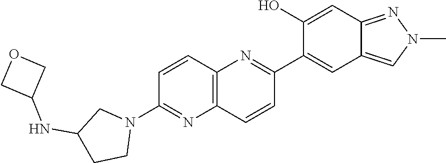 [SMILES: Cn1cc2cc(-c3ccc4nc(N5CCC(NC6COC6)C5)ccc4n3)c(O)cc2n1] |
| 268 | 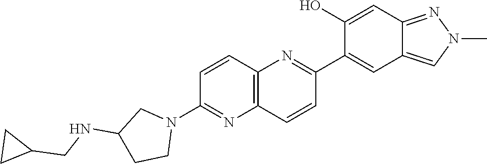 [SMILES: Cn1cc2cc(-c3ccc4nc(N5CCC(NCC6CC6)C5)ccc4n3)c(O)cc2n1] |
| 269 | 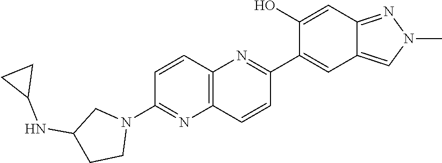 [SMILES: Cn1cc2cc(-c3ccc4nc(N5CCC(NC6CC6)C5)ccc4n3)c(O)cc2n1] |
| 270 | 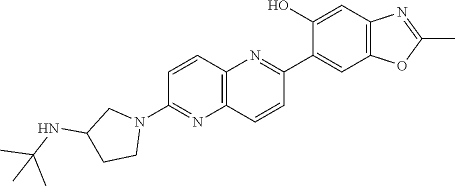 [SMILES: Cc1nc2cc(O)c(-c3ccc4nc(N5CCC(NC(C)(C)C)C5)ccc4n3)cc2o1] |
| 271 | 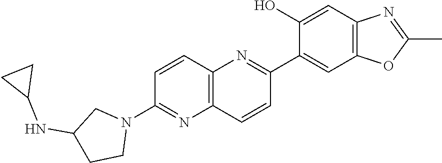 [SMILES: Cc1nc2cc(O)c(-c3ccc4nc(N5CCC(NC6CC6)C5)ccc4n3)cc2o1] |
| 272 | 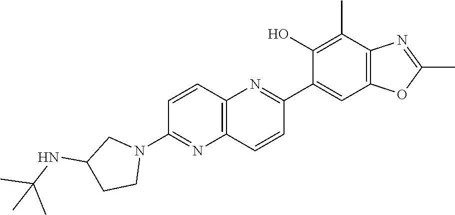 [SMILES: Cc1nc2c(C)c(O)c(-c3ccc4nc(N5CCC(NC(C)(C)C)C5)ccc4n3)cc2o1] |
| 273 | 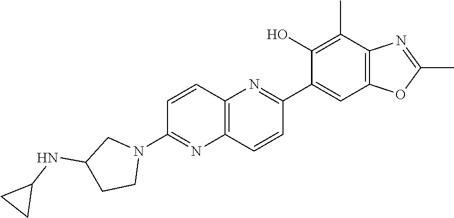 [SMILES: Cc1nc2c(C)c(O)c(-c3ccc4nc(N5CCC(NC6CC6)C5)ccc4n3)cc2o1] |
| 274 | 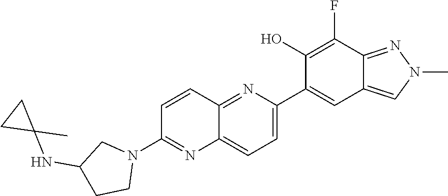 [SMILES: Cn1cc2cc(-c3ccc4nc(N5CCC(NC6(C)CC6)C5)ccc4n3)c(O)c(F)c2n1] |
| 275 | 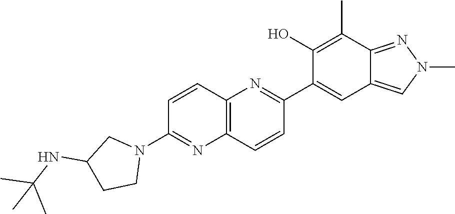 [SMILES: Cc1c(O)c(-c2ccc3nc(N4CCC(NC(C)(C)C)C4)ccc3n2)cc2cn(C)nc12] |
| 277 | 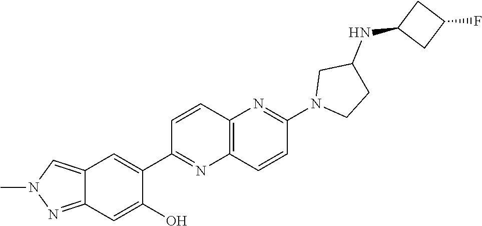 [SMILES: Cn1cc2cc(-c3ccc4nc(N5CCC(N[C@H]6C[C@H](F)C6)C5)ccc4n3)c(O)cc2n1] |
| 278 | 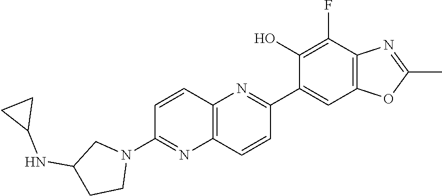 [SMILES: Cc1nc2c(F)c(O)c(-c3ccc4nc(N5CCC(NC6CC6)C5)ccc4n3)cc2o1] |
| 279 | 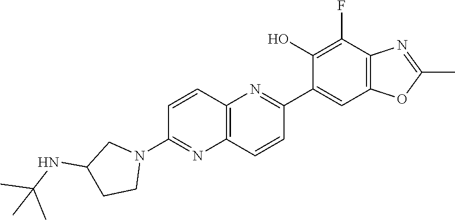 [SMILES: Cc1nc2c(F)c(O)c(-c3ccc4nc(N5CCC(NC(C)(C)C)C5)ccc4n3)cc2o1] |
| 280 | 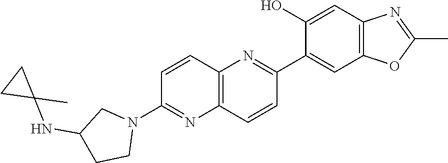 [SMILES: Cc1nc2cc(O)c(-c3ccc4nc(N5CCC(NC6(C)CC6)C5)ccc4n3)cc2o1] |
| 281 | 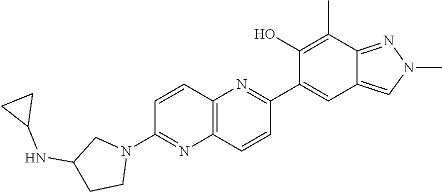 [SMILES: Cc1c(O)c(-c2ccc3nc(N4CCC(NC5CC5)C4)ccc3n2)cc2cn(C)nc12] |
| 282 | 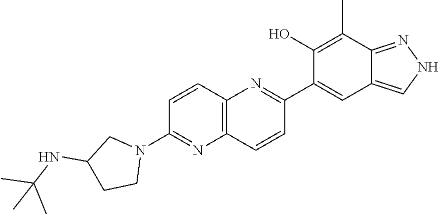 [SMILES: Cc1c(O)c(-c2ccc3nc(N4CCC(NC(C)(C)C)C4)ccc3n2)cc2c[nH]nc12] |
| 283 | 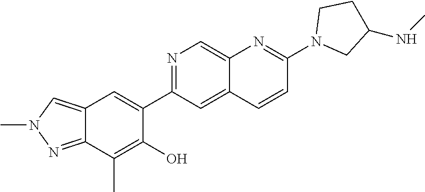 |
| 284 | 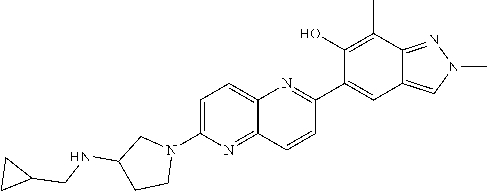 [SMILES: Cc1c(O)c(-c2ccc3nc(N4CCC(NCC5CC5)C4)ccc3n2)cc2cn(C)nc12] |
| 285 | 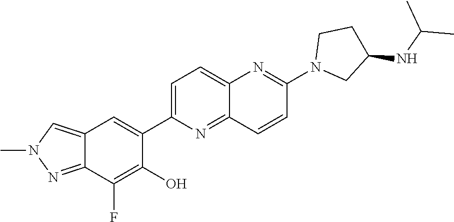 [SMILES: CC(C)N[C@@H]1CCN(c2ccc3nc(-c4cc5cn(C)nc5c(F)c4O)ccc3n2)C1] |
| 286 | 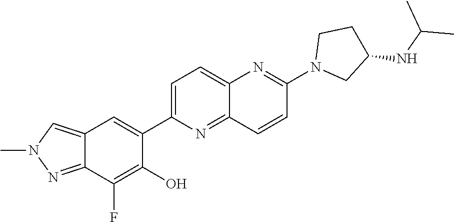 [SMILES: CC(C)N[C@H]1CCN(c2ccc3nc(-c4cc5cn(C)nc5c(F)c4O)ccc3n2)C1] |
| 287 | 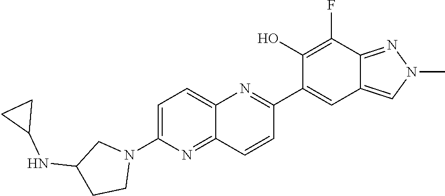 [SMILES: Cn1cc2cc(-c3ccc4nc(N5CCC(NC6CC6)C5)ccc4n3)c(O)c(F)c2n1] |
| 288 | 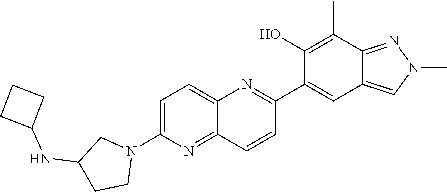 [SMILES: Cc1c(O)c(-c2ccc3nc(N4CCC(NC5CCC5)C4)ccc3n2)cc2cn(C)nc12] |
| 289 | 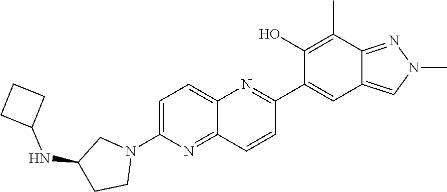 [SMILES: Cc1c(O)c(-c2ccc3nc(N4CC[C@@H](NC5CCC5)C4)ccc3n2)cc2cn(C)nc12] |
| 290 | 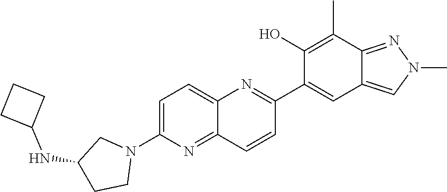 [SMILES: Cc1c(O)c(-c2ccc3nc(N4CC[C@H](NC5CCC5)C4)ccc3n2)cc2cn(C)nc12] |
| 291 | 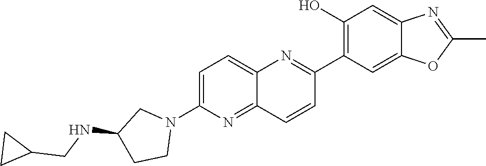 [SMILES: Cc1nc2cc(O)c(-c3ccc4nc(N5CC[C@@H](NCC6CC6)C5)ccc4n3)cc2o1] |
| 292 | 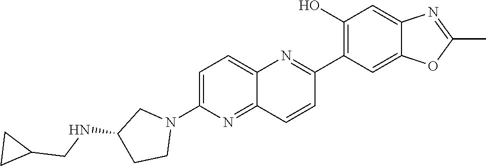 [SMILES: Cc1nc2cc(O)c(-c3ccc4nc(N5CC[C@H](NCC6CC6)C5)ccc4n3)cc2o1] |
| 293 | 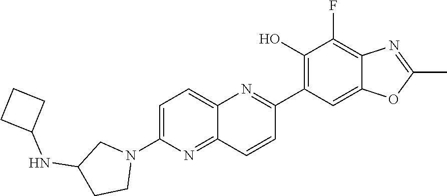 [SMILES: Cc1nc2c(F)c(O)c(-c3ccc4nc(N5CCC(NC6CCC6)C5)ccc4n3)cc2o1] |
| 294 | 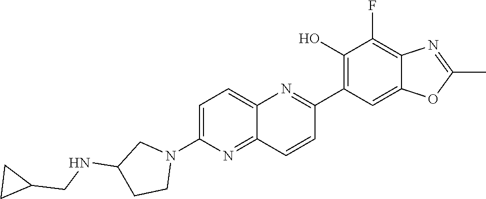 [SMILES: Cc1nc2c(F)c(O)c(-c3ccc4nc(N5CCC(NCC6CC6)C5)ccc4n3)cc2o1] |
| 295 | 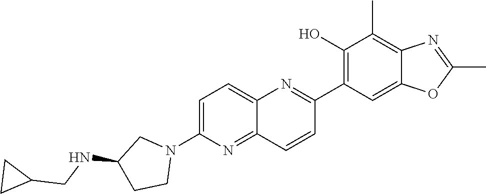 [SMILES: Cc1nc2c(C)c(O)c(-c3ccc4nc(N5CC[C@@H](NCC6CC6)C5)ccc4n3)cc2o1] |
| 296 | 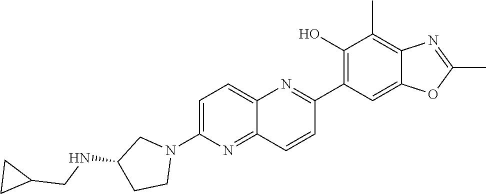 [SMILES: Cc1nc2c(C)c(O)c(-c3ccc4nc(N5CC[C@H](NCC6CC6)C5)ccc4n3)cc2o1] |
| 297 | 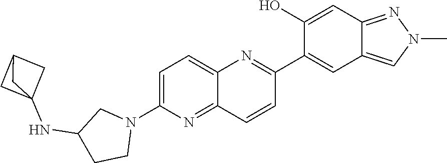 [SMILES: Cn1cc2cc(-c3ccc4nc(N5CCC(NC67CC(C6)C7)C5)ccc4n3)c(O)cc2n1] |
| 298 | 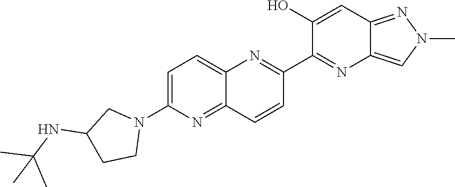 [SMILES: Cn1cc2nc(-c3ccc4nc(N5CCC(NC(C)(C)C)C5)ccc4n3)c(O)cc2n1] |
| 299 | 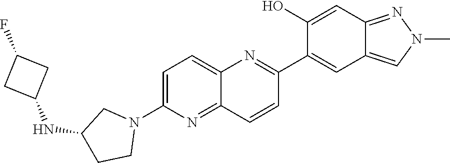 [SMILES: Cn1cc2cc(-c3ccc4nc(N5CC[C@H](N[C@H]6C[C@@H](F)C6)C5)ccc4n3)c(O)cc2n1] |
| 300 | 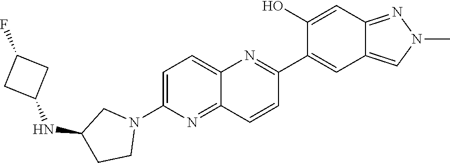 [SMILES: Cn1cc2cc(-c3ccc4nc(N5CC[C@@H](N[C@H]6C[C@@H](F)C6)C5)ccc4n3)c(O)cc2n1] |
| 301 | 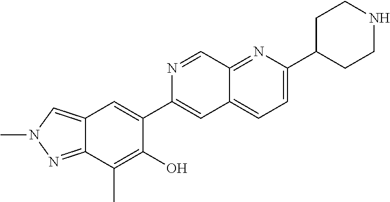 [SMILES: Cc1c(O)c(-c2cc3ccc(C4CCNCC4)nc3cn2)cc2cn(C)nc12] |
| 302 | 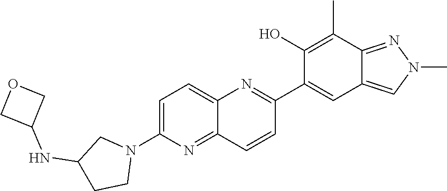 [SMILES: Cc1c(O)c(-c2ccc3nc(N4CCC(NC5COC5)C4)ccc3n2)cc2cn(C)nc12] |
| 303 | 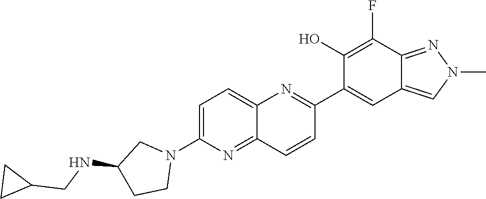 [SMILES: Cn1cc2cc(-c3ccc4nc(N5CC[C@@H](NCC6CC6)C5)ccc4n3)c(O)c(F)c2n1] |
| 304 | 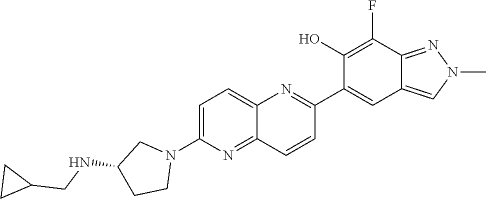 [SMILES: Cn1cc2cc(-c3ccc4nc(N5CC[C@H](NCC6CC6)C5)ccc4n3)c(O)c(F)c2n1] |
| 305 | 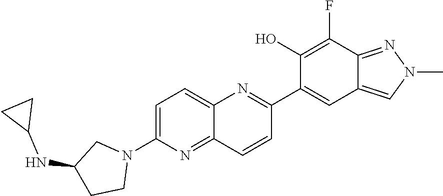 [SMILES: Cn1cc2cc(-c3ccc4nc(N5CC[C@@H](NC6CC6)C5)ccc4n3)c(O)c(F)c2n1] |
| 306 | 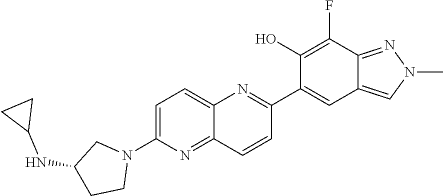 [SMILES: Cn1cc2cc(-c3ccc4nc(N5CC[C@H](NC6CC6)C5)ccc4n3)c(O)c(F)c2n1] |
| 307 | 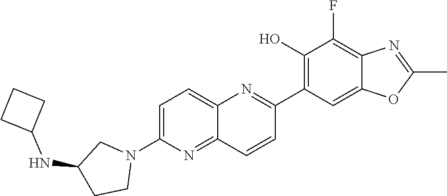 [SMILES: Cc1nc2c(F)c(O)c(-c3ccc4nc(N5CC[C@@H](NC6CCC6)C5)ccc4n3)cc2o1] |
| 308 | 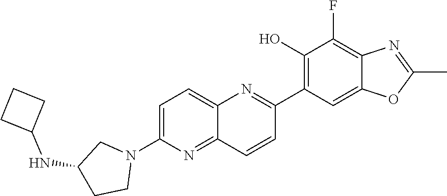 [SMILES: Cc1nc2c(F)c(O)c(-c3ccc4nc(N5CC[C@H](NC6CCC6)C5)ccc4n3)cc2o1] |
| 309 | 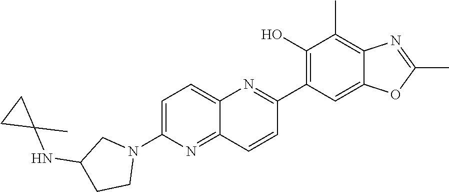 [SMILES: Cc1nc2c(C)c(O)c(-c3ccc4nc(N5CCC(NC6(C)CC6)C5)ccc4n3)cc2o1] |
| 310 | 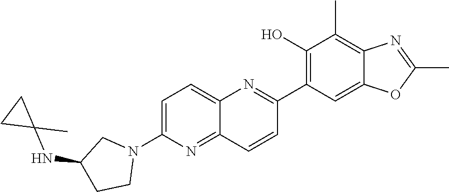 [SMILES: Cc1nc2c(C)c(O)c(-c3ccc4nc(N5CC[C@@H](NC6(C)CC6)C5)ccc4n3)cc2o1] |
| 311 | 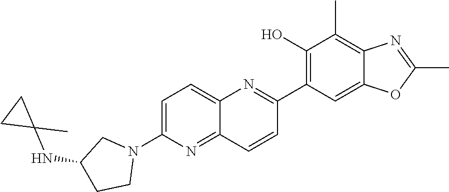 [SMILES: Cc1nc2c(C)c(O)c(-c3ccc4nc(N5CC[C@H](NC6(C)CC6)C5)ccc4n3)cc2o1] |
| 312 | 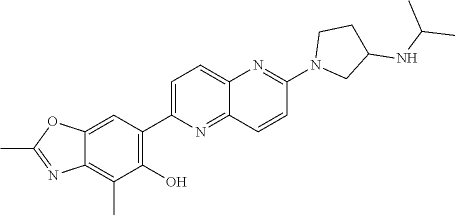 [SMILES: Cc1nc2c(C)c(O)c(-c3ccc4nc(N5CCC(NC(C)C)C5)ccc4n3)cc2o1] |
| 313 | 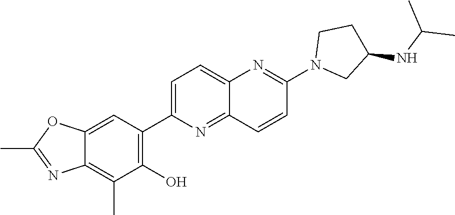 [SMILES: Cc1nc2c(C)c(O)c(-c3ccc4nc(N5CC[C@@H](NC(C)C)C5)ccc4n3)cc2o1] |
| 314 | 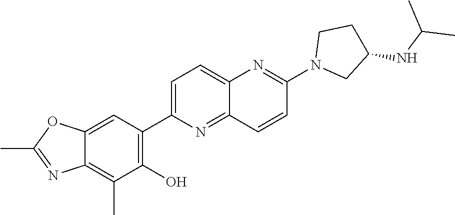 [SMILES: Cc1nc2c(C)c(O)c(-c3ccc4nc(N5CC[C@H](NC(C)C)C5)ccc4n3)cc2o1] |
| 315 | 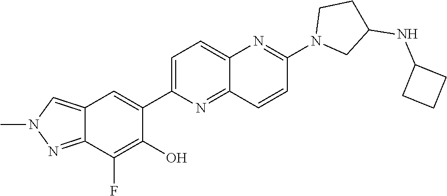 [SMILES: Cn1cc2cc(-c3ccc4nc(N5CCC(NC6CCC6)C5)ccc4n3)c(O)c(F)c2n1] |
| 316 | 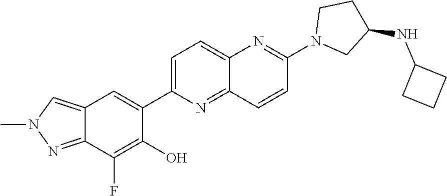 [SMILES: Cn1cc2cc(-c3ccc4nc(N5CC[C@@H](NC6CCC6)C5)ccc4n3)c(O)c(F)c2n1] |
| 317 | 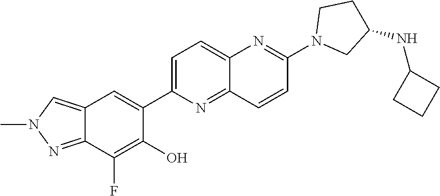 [SMILES: Cn1cc2cc(-c3ccc4nc(N5CC[C@H](NC6CCC6)C5)ccc4n3)c(O)c(F)c2n1] |
| 318 | 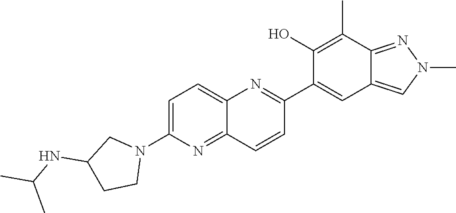 [SMILES: Cc1c(O)c(-c2ccc3nc(N4CCC(NC(C)C)C4)ccc3n2)cc2cn(C)nc12] |
| 319 | 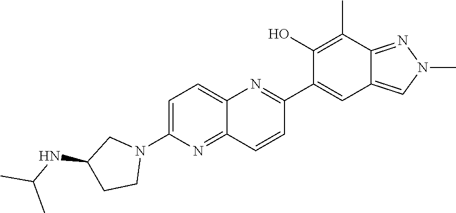 [SMILES: Cc1c(O)c(-c2ccc3nc(N4CC[C@@H](NC(C)C)C4)ccc3n2)cc2cn(C)nc12] |
| 320 | 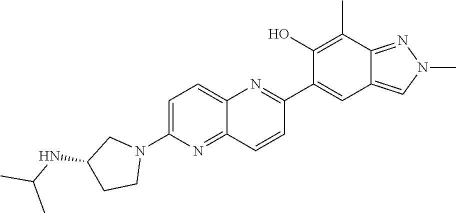 [SMILES: Cc1c(O)c(-c2ccc3nc(N4CC[C@H](NC(C)C)C4)ccc3n2)cc2cn(C)nc12] |
| 321 | 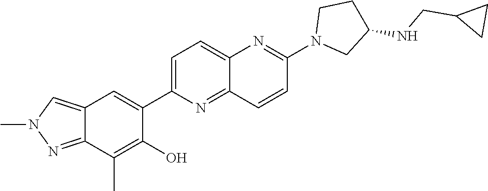 [SMILES: Cc1c(O)c(-c2ccc3nc(N4CC[C@H](NCC5CC5)C4)ccc3n2)cc2cn(C)nc12] |
| 322 | 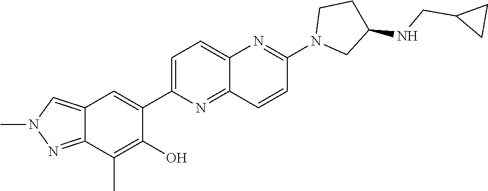 [SMILES: Cc1c(O)c(-c2ccc3nc(N4CC[C@@H](NCC5CC5)C4)ccc3n2)cc2cn(C)nc12] |
| 323 | 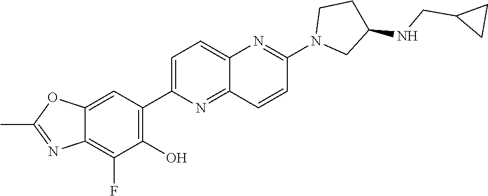 [SMILES: Cc1nc2c(F)c(O)c(-c3ccc4nc(N5CC[C@@H](NCC6CC6)C5)ccc4n3)cc2o1] |
| 324 | 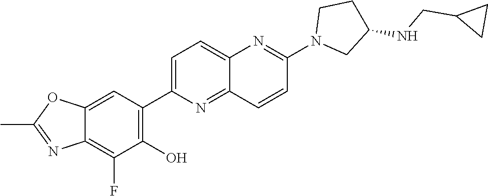 [SMILES: Cc1nc2c(F)c(O)c(-c3ccc4nc(N5CC[C@H](NCC6CC6)C5)ccc4n3)cc2o1] |
| 325 | 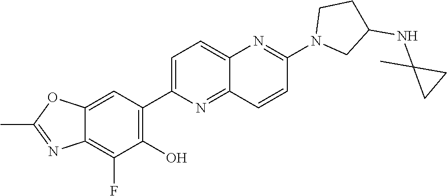 [SMILES: Cc1nc2c(F)c(O)c(-c3ccc4nc(N5CCC(NC6(C)CC6)C5)ccc4n3)cc2o1] |
| 326 | 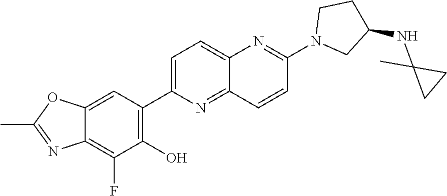 [SMILES: Cc1nc2c(F)c(O)c(-c3ccc4nc(N5CC[C@@H](NC6(C)CC6)C5)ccc4n3)cc2o1] |
| 327 | 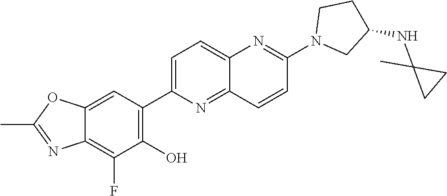 [SMILES: Cc1nc2c(F)c(O)c(-c3ccc4nc(N5CC[C@H](NC6(C)CC6)C5)ccc4n3)cc2o1] |
| 328 | 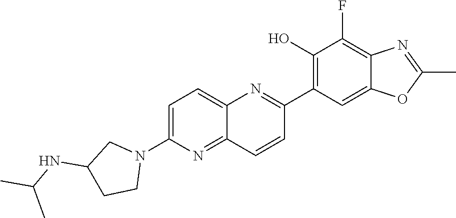 [SMILES: Cc1nc2c(F)c(O)c(-c3ccc4nc(N5CCC(NC(C)C)C5)ccc4n3)cc2o1] |
| 329 | 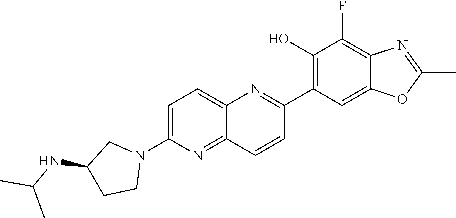 [SMILES: Cc1nc2c(F)c(O)c(-c3ccc4nc(N5CC[C@@H](NC(C)C)C5)ccc4n3)cc2o1] |
| 330 | 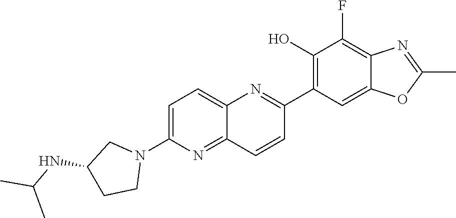 [SMILES: Cc1nc2c(F)c(O)c(-c3ccc4nc(N5CC[C@H](NC(C)C)C5)ccc4n3)cc2o1] |
| 331 | 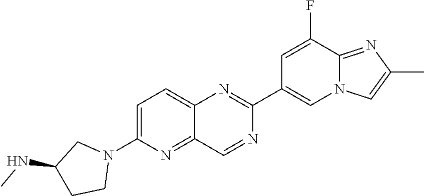 |
| 332 | 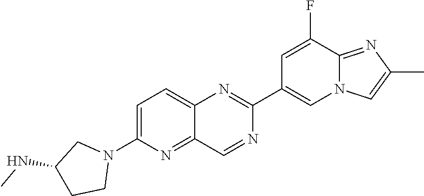 |
| 333 | 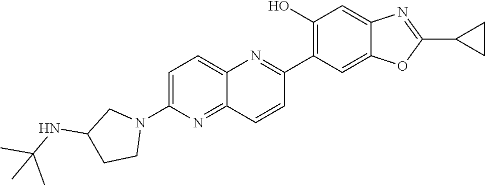 [SMILES: CC(C)(C)NC1CCN(c2ccc3nc(-c4cc5oc(C6CC6)nc5cc4O)ccc3n2)C1] |
| 334 | 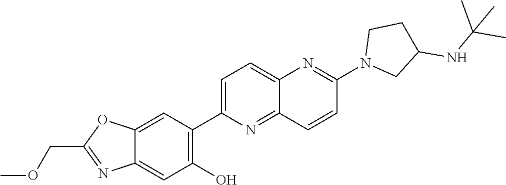 [SMILES: COCc1nc2cc(O)c(-c3ccc4nc(N5CCC(NC(C)(C)C)C5)ccc4n3)cc2o1] |
| 335 | 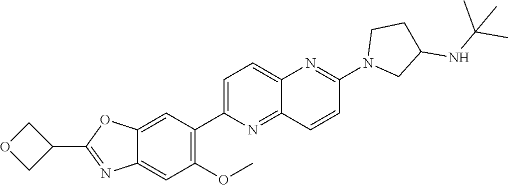 [SMILES: COc1cc2nc(C3COC3)oc2cc1-c1ccc2nc(N3CCC(NC(C)(C)C)C3)ccc2n1] |
| 336 | 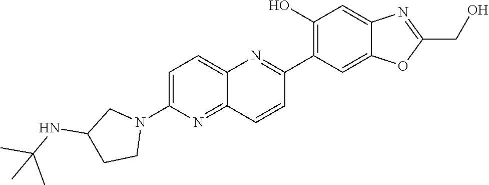 [SMILES: CC(C)(C)NC1CCN(c2ccc3nc(-c4cc5oc(CO)nc5cc4O)ccc3n2)C1] |
| 337 | 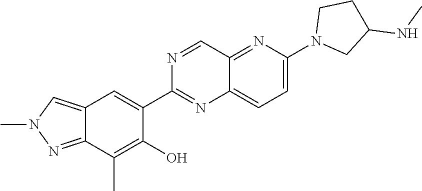 |
| 338 | 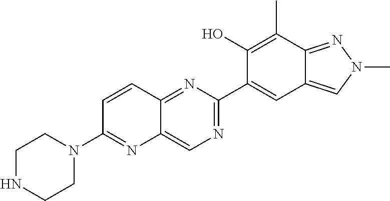 [SMILES: Cc1c(O)c(-c2ncc3nc(N4CCNCC4)ccc3n2)cc2cn(C)nc12] |
| 339 | 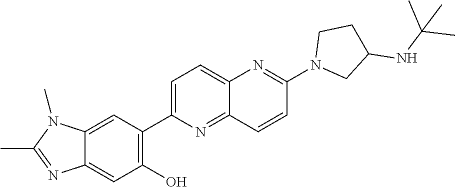 [SMILES: Cc1nc2cc(O)c(-c3ccc4nc(N5CCC(NC(C)(C)C)C5)ccc4n3)cc2n1C] |
| 340 | 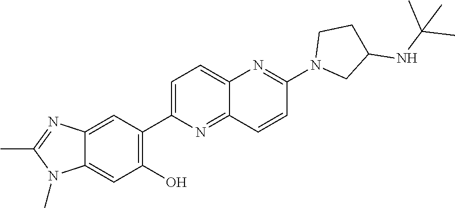 [SMILES: Cc1nc2cc(-c3ccc4nc(N5CCC(NC(C)(C)C)C5)ccc4n3)c(O)cc2n1C] |
| 341 | 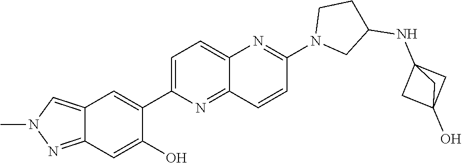 [SMILES: Cn1cc2cc(-c3ccc4nc(N5CCC(NC67CC(O)(C6)C7)C5)ccc4n3)c(O)cc2n1] |
| 342 | 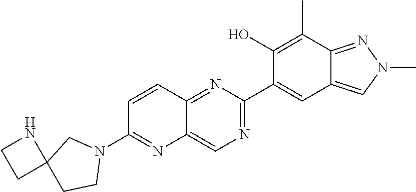 [SMILES: Cc1c(O)c(-c2ncc3nc(N4CCC5(CCN5)C4)ccc3n2)cc2cn(C)nc12] |
| 343 | 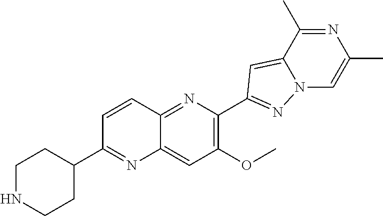 [SMILES: COc1cc2nc(C3CCNCC3)ccc2nc1-c1cc2c(C)nc(C)cn2n1] |
| 344 | 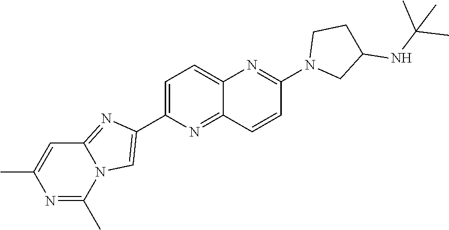 [SMILES: Cc1cc2nc(-c3ccc4nc(N5CCC(NC(C)(C)C)C5)ccc4n3)cn2c(C)n1] |
| 345 | 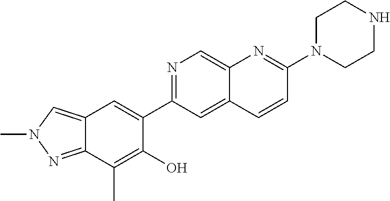 [SMILES: Cc1c(O)c(-c2cc3ccc(N4CCNCC4)nc3cn2)cc2cn(C)nc12] |
| 346 | 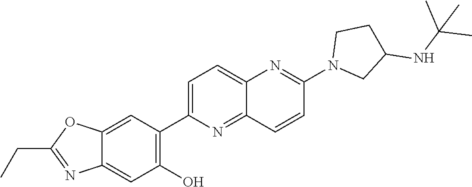 [SMILES: CCc1nc2cc(O)c(-c3ccc4nc(N5CCC(NC(C)(C)C)C5)ccc4n3)cc2o1] |
| 347 | 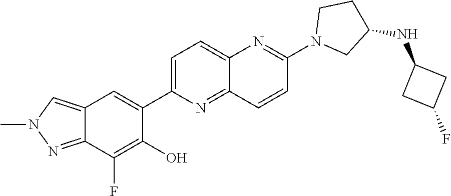 [SMILES: Cn1cc2cc(-c3ccc4nc(N5CC[C@H](N[C@H]6C[C@H](F)C6)C5)ccc4n3)c(O)c(F)c2n1] |
| 348 | 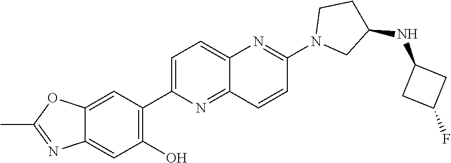 [SMILES: Cc1nc2cc(O)c(-c3ccc4nc(N5CC[C@@H](N[C@H]6C[C@H](F)C6)C5)ccc4n3)cc2o1] |
| 349 | 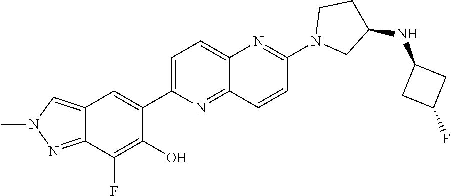 [SMILES: Cn1cc2cc(-c3ccc4nc(N5CC[C@@H](N[C@H]6C[C@H](F)C6)C5)ccc4n3)c(O)c(F)c2n1] |
| 350 | 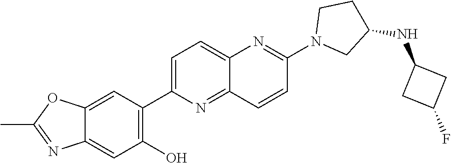 [SMILES: Cc1nc2cc(O)c(-c3ccc4nc(N5CC[C@H](N[C@H]6C[C@H](F)C6)C5)ccc4n3)cc2o1] |
| 351 | 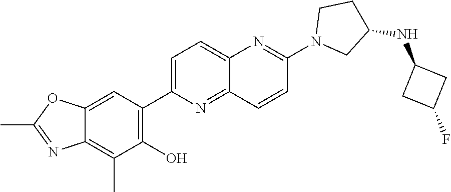 [SMILES: Cc1nc2c(C)c(O)c(-c3ccc4nc(N5CC[C@H](N[C@H]6C[C@H](F)C6)C5)ccc4n3)cc2o1] |
| 352 | 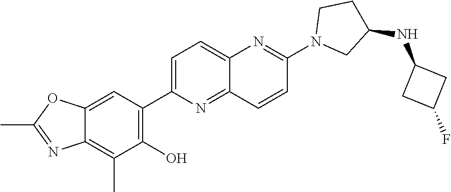 [SMILES: Cc1nc2c(C)c(O)c(-c3ccc4nc(N5CC[C@@H](N[C@H]6C[C@H](F)C6)C5)ccc4n3)cc2o1] |
| 353 | 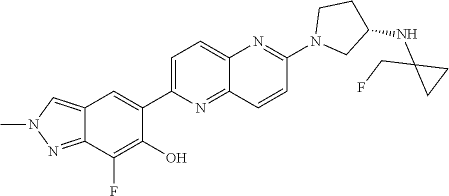 [SMILES: Cn1cc2cc(-c3ccc4nc(N5CC[C@H](NC6(CF)CC6)C5)ccc4n3)c(O)c(F)c2n1] |
| 354 | 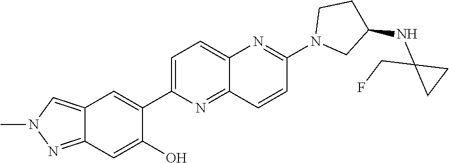 [SMILES: Cn1cc2cc(-c3ccc4nc(N5CC[C@@H](NC6(CF)CC6)C5)ccc4n3)c(O)cc2n1] |
| 355 | 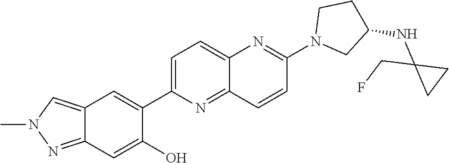 [SMILES: Cn1cc2cc(-c3ccc4nc(N5CC[C@H](NC6(CF)CC6)C5)ccc4n3)c(O)cc2n1] |
| 356 | 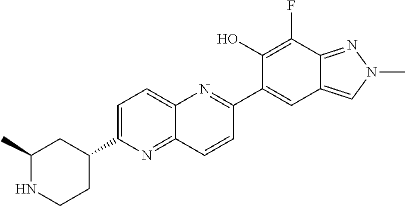 |
| 357 | 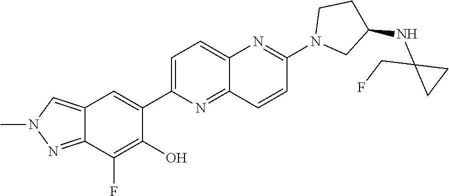 [SMILES: Cn1cc2cc(-c3ccc4nc(N5CC[C@@H](NC6(CF)CC6)C5)ccc4n3)c(O)c(F)c2n1] |
| 358 | 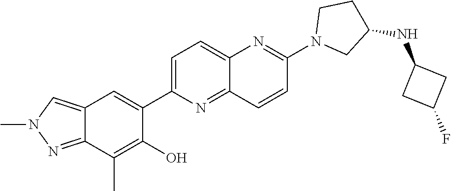 [SMILES: Cc1c(O)c(-c2ccc3nc(N4CC[C@H](N[C@H]5C[C@H](F)C5)C4)ccc3n2)cc2cn(C)nc12] |
| 359 | 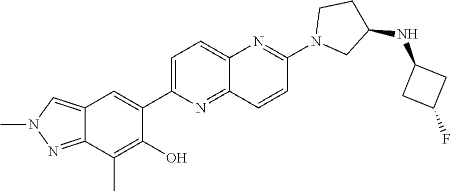 [SMILES: Cc1c(O)c(-c2ccc3nc(N4CC[C@@H](N[C@H]5C[C@H](F)C5)C4)ccc3n2)cc2cn(C)nc12] |
| 360 | 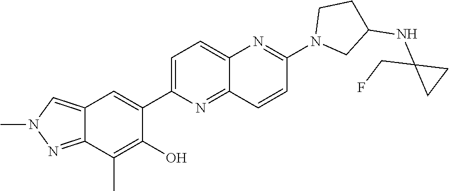 [SMILES: Cc1c(O)c(-c2ccc3nc(N4CCC(NC5(CF)CC5)C4)ccc3n2)cc2cn(C)nc12] |
| 361 | 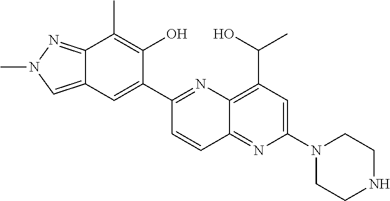 [SMILES: Cc1c(O)c(-c2ccc3nc(N4CCNCC4)cc(C(C)O)c3n2)cc2cn(C)nc12] |
| 362 | 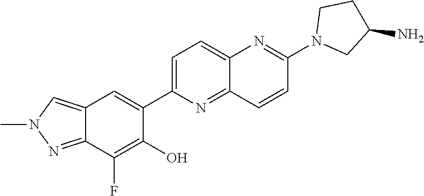 [SMILES: Cn1cc2cc(-c3ccc4nc(N5CC[C@@H](N)C5)ccc4n3)c(O)c(F)c2n1] |
| 363 | 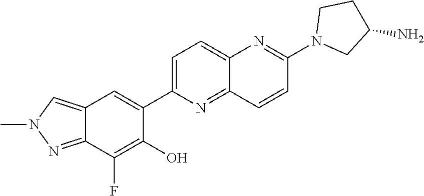 [SMILES: Cn1cc2cc(-c3ccc4nc(N5CC[C@H](N)C5)ccc4n3)c(O)c(F)c2n1] |
| 364 | 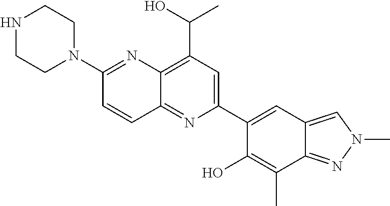 [SMILES: Cc1c(O)c(-c2cc(C(C)O)c3nc(N4CCNCC4)ccc3n2)cc2cn(C)nc12] |
| 365 | 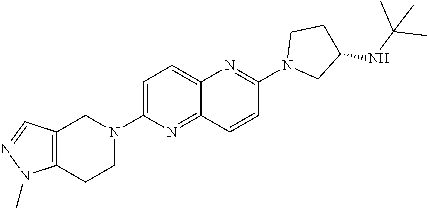 [SMILES: Cn1ncc2c1CCN(c1ccc3nc(N4CC[C@H](NC(C)(C)C)C4)ccc3n1)C2] |
| 366 | 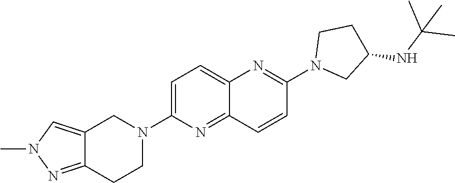 [SMILES: Cn1cc2c(n1)CCN(c1ccc3nc(N4CC[C@H](NC(C)(C)C)C4)ccc3n1)C2] |
| 367 | 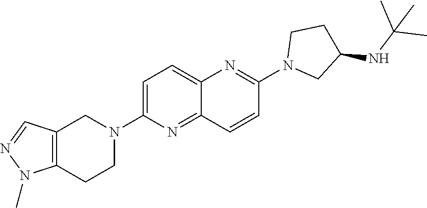 [SMILES: Cn1ncc2c1CCN(c1ccc3nc(N4CC[C@@H](NC(C)(C)C)C4)ccc3n1)C2] |
| 368 | 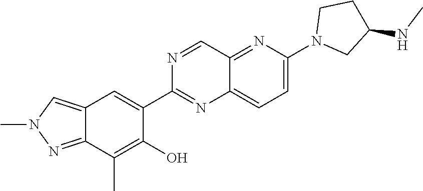 |
| 369 | 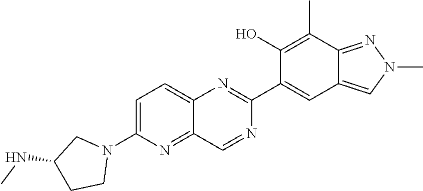 |
| 370 | 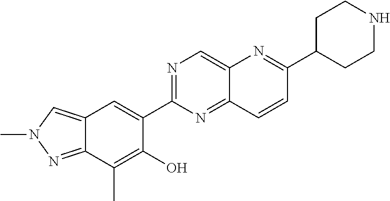 [SMILES: Cc1c(O)c(-c2ncc3nc(C4CCNCC4)ccc3n2)cc2cn(C)nc12] |
| 371 | 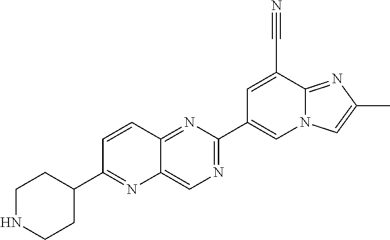 [SMILES: Cc1cn2cc(-c3ncc4nc(C5CCNCC5)ccc4n3)cc(C#N)c2n1] |
| 372 | 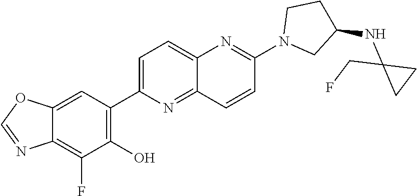 [SMILES: Oc1c(-c2ccc3nc(N4CC[C@@H](NC5(CF)CC5)C4)ccc3n2)cc2ocnc2c1F] |
| 373 | 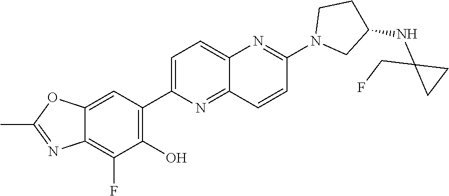 [SMILES: Cc1nc2c(F)c(O)c(-c3ccc4nc(N5CC[C@H](NC6(CF)CC6)C5)ccc4n3)cc2o1] |
| 374 | 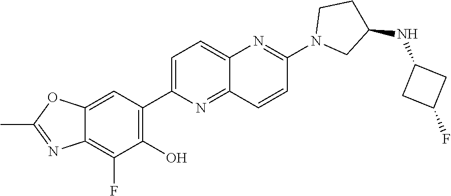 [SMILES: Cc1nc2c(F)c(O)c(-c3ccc4nc(N5CC[C@@H](N[C@H]6C[C@@H](F)C6)C5)ccc4n3)cc2o1] |
| 375 | 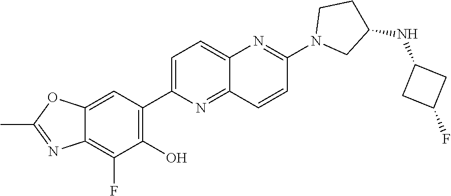 [SMILES: Cc1nc2c(F)c(O)c(-c3ccc4nc(N5CC[C@H](N[C@H]6C[C@@H](F)C6)C5)ccc4n3)cc2o1] |
| 376 | 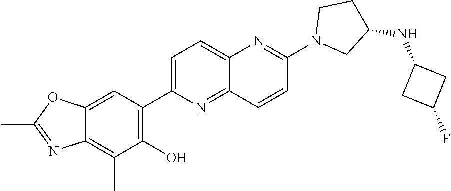 [SMILES: Cc1nc2c(C)c(O)c(-c3ccc4nc(N5CC[C@H](N[C@H]6C[C@@H](F)C6)C5)ccc4n3)cc2o1] |
| 377 | 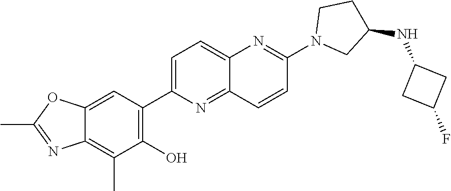 [SMILES: Cc1nc2c(C)c(O)c(-c3ccc4nc(N5CC[C@@H](N[C@H]6C[C@@H](F)C6)C5)ccc4n3)cc2o1] |
| 378 | 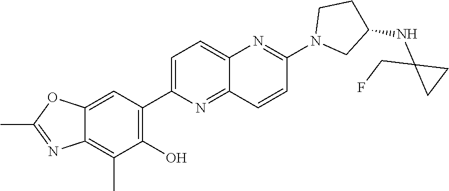 [SMILES: Cc1nc2c(C)c(O)c(-c3ccc4nc(N5CC[C@H](NC6(CF)CC6)C5)ccc4n3)cc2o1] |
| 379 | 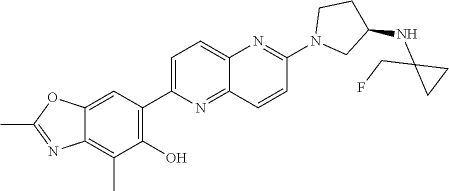 [SMILES: Cc1nc2c(C)c(O)c(-c3ccc4nc(N5CC[C@@H](NC6(CF)CC6)C5)ccc4n3)cc2o1] |
| 380 | 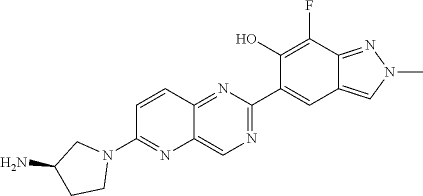 [SMILES: Cn1cc2cc(-c3ncc4nc(N5CC[C@@H](N)C5)ccc4n3)c(O)c(F)c2n1] |
| 381 | 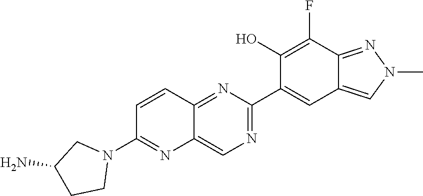 [SMILES: Cn1cc2cc(-c3ncc4nc(N5CC[C@H](N)C5)ccc4n3)c(O)c(F)c2n1] |
| 382 | 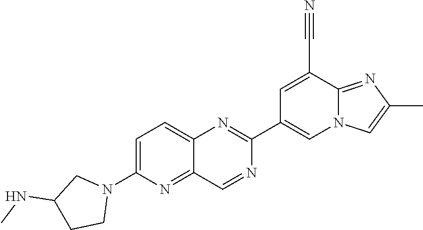 |
| 383 | 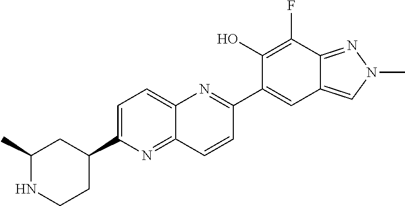 |
| 384 | 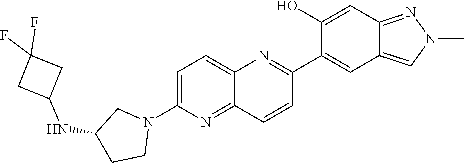 [SMILES: Cn1cc2cc(-c3ccc4nc(N5CC[C@H](NC6CC(F)(F)C6)C5)ccc4n3)c(O)cc2n1] |
| 385 | 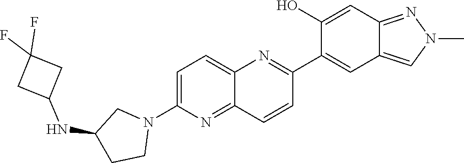 [SMILES: Cn1cc2cc(-c3ccc4nc(N5CC[C@@H](NC6CC(F)(F)C6)C5)ccc4n3)c(O)cc2n1] |
| 386 | 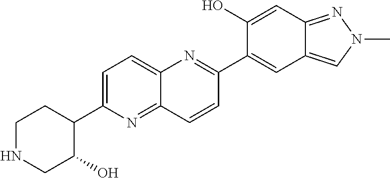 [SMILES: Cn1cc2cc(-c3ccc4nc(C5CCNC[C@H]5O)ccc4n3)c(O)cc2n1] |
| 387 | 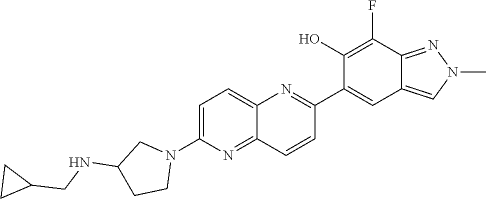 [SMILES: Cn1cc2cc(-c3ccc4nc(N5CCC(NCC6CC6)C5)ccc4n3)c(O)c(F)c2n1] |
| 388 | 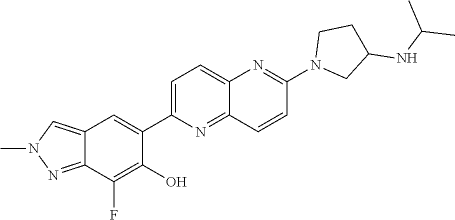 [SMILES: CC(C)NC1CCN(c2ccc3nc(-c4cc5cn(C)nc5c(F)c4O)ccc3n2)C1] |
| 389 | 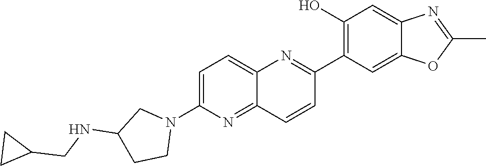 [SMILES: Cc1nc2cc(O)c(-c3ccc4nc(N5CCC(NCC6CC6)C5)ccc4n3)cc2o1] |
| 390 | 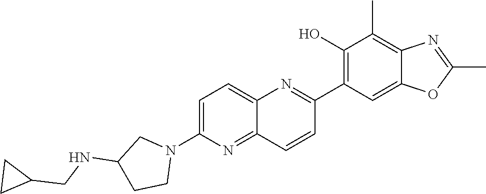 [SMILES: Cc1nc2c(C)c(O)c(-c3ccc4nc(N5CCC(NCC6CC6)C5)ccc4n3)cc2o1] |
| 391 | 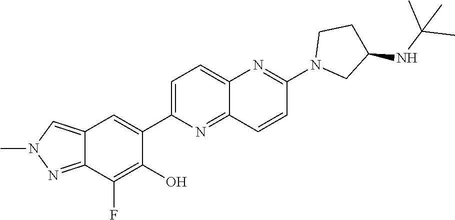 [SMILES: Cn1cc2cc(-c3ccc4nc(N5CC[C@@H](NC(C)(C)C)C5)ccc4n3)c(O)c(F)c2n1] |
| 392 | 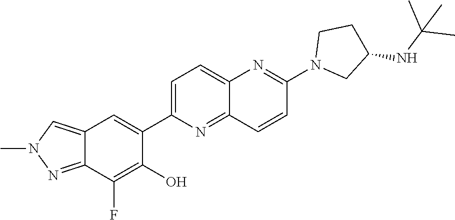 [SMILES: Cn1cc2cc(-c3ccc4nc(N5CC[C@H](NC(C)(C)C)C5)ccc4n3)c(O)c(F)c2n1] |
| 393 | 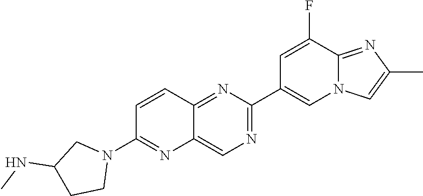 |
| 395 | 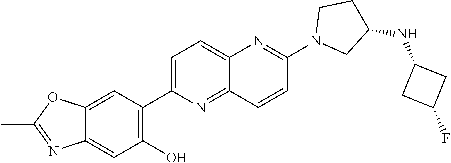 [SMILES: Cc1nc2cc(O)c(-c3ccc4nc(N5CC[C@H](N[C@H]6C[C@@H](F)C6)C5)ccc4n3)cc2o1] |
| 396 | 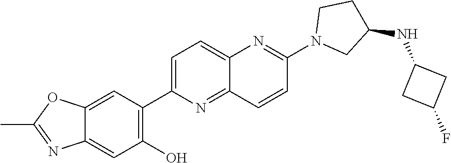 [SMILES: Cc1nc2cc(O)c(-c3ccc4nc(N5CC[C@@H](N[C@H]6C[C@@H](F)C6)C5)ccc4n3)cc2o1] |
| 397 | 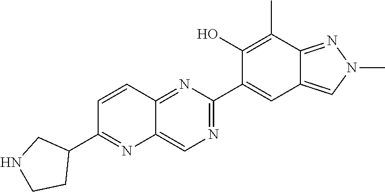 [SMILES: Cc1c(O)c(-c2ncc3nc(C4CCNC4)ccc3n2)cc2cn(C)nc12] |
| 398 | 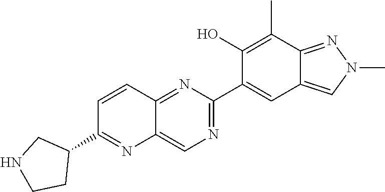 [SMILES: Cc1c(O)c(-c2ncc3nc([C@@H]4CCNC4)ccc3n2)cc2cn(C)nc12] |
| 399 | 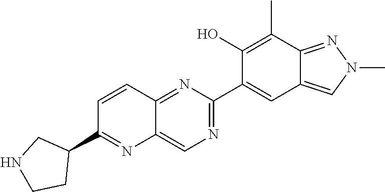 [SMILES: Cc1c(O)c(-c2ncc3nc([C@H]4CCNC4)ccc3n2)cc2cn(C)nc12] |
| 400 | 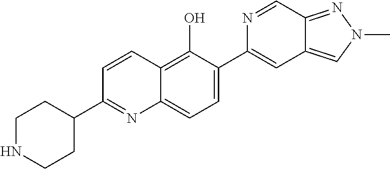 [SMILES: Cn1cc2cc(-c3ccc4nc(C5CCNCC5)ccc4c3O)ncc2n1] |
| 401 | 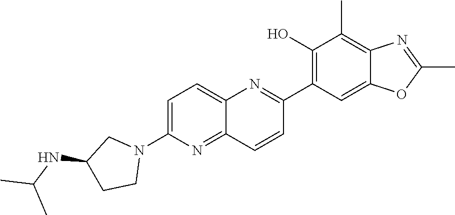 [SMILES: Cc1nc2c(C)c(O)c(-c3ccc4nc(N5CC[C@@H](NC(C)C)C5)ccc4n3)cc2o1] |
| 402 | 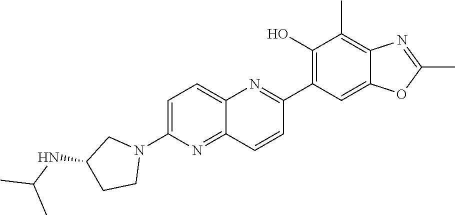 [SMILES: Cc1nc2c(C)c(O)c(-c3ccc4nc(N5CC[C@H](NC(C)C)C5)ccc4n3)cc2o1] |
| 403 | 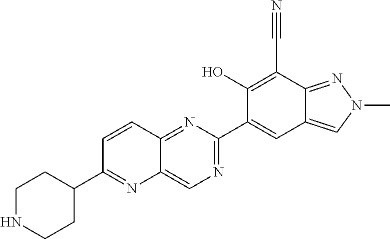 [SMILES: Cn1cc2cc(-c3ncc4nc(C5CCNCC5)ccc4n3)c(O)c(C#N)c2n1] |
| 404 | 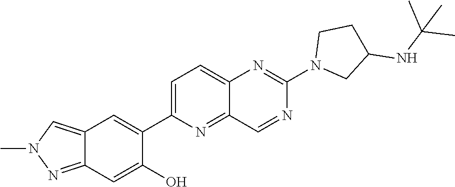 [SMILES: Cn1cc2cc(-c3ccc4nc(N5CCC(NC(C)(C)C)C5)ncc4n3)c(O)cc2n1] |
| 405 | 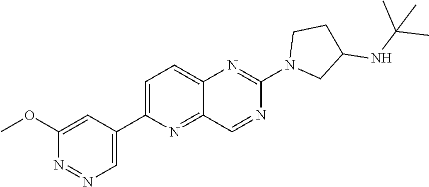 [SMILES: COc1cc(-c2ccc3nc(N4CCC(NC(C)(C)C)C4)ncc3n2)cnn1] |
| 406 | 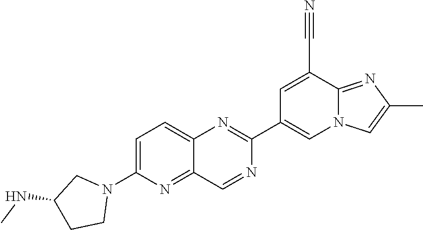 |
| 407 | 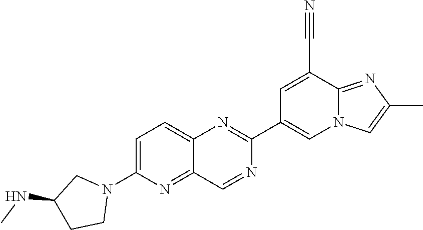 |
| 408 | 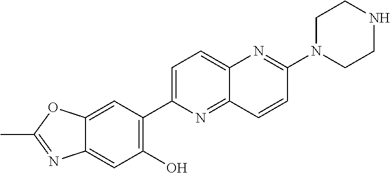 [SMILES: Cc1nc2cc(O)c(-c3ccc4nc(N5CCNCC5)ccc4n3)cc2o1] |
| 409 | 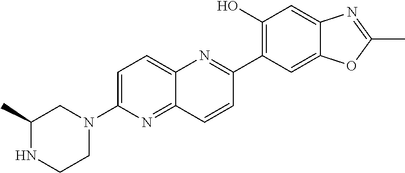 |
| 410 | 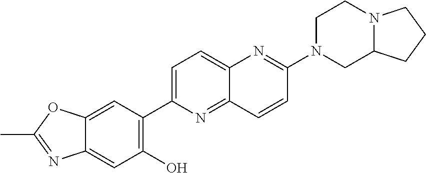 [SMILES: Cc1nc2cc(O)c(-c3ccc4nc(N5CCN6CCCC6C5)ccc4n3)cc2o1] |
| 411 | 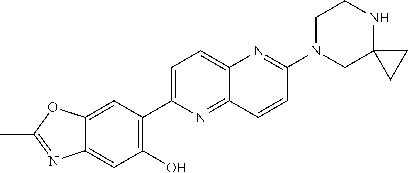 [SMILES: Cc1nc2cc(O)c(-c3ccc4nc(N5CCNC6(CC6)C5)ccc4n3)cc2o1] |
| 412 | 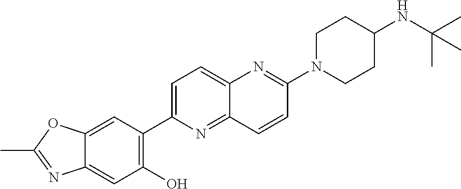 [SMILES: Cc1nc2cc(O)c(-c3ccc4nc(N5CCC(NC(C)(C)C)CC5)ccc4n3)cc2o1] |
| 413 | 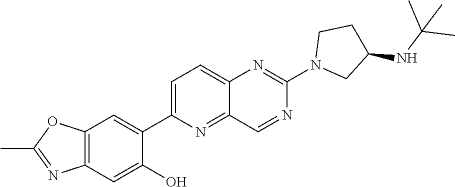 [SMILES: Cc1nc2cc(O)c(-c3ccc4nc(N5CC[C@@H](NC(C)(C)C)C5)ncc4n3)cc2o1] |
| 414 | 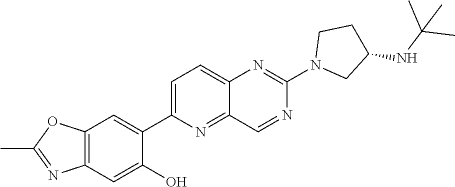 [SMILES: Cc1nc2cc(O)c(-c3ccc4nc(N5CC[C@H](NC(C)(C)C)C5)ncc4n3)cc2o1] |
| 415 | 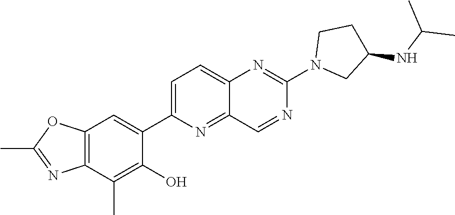 [SMILES: Cc1nc2c(C)c(O)c(-c3ccc4nc(N5CC[C@@H](NC(C)C)C5)ncc4n3)cc2o1] |
| 416 | 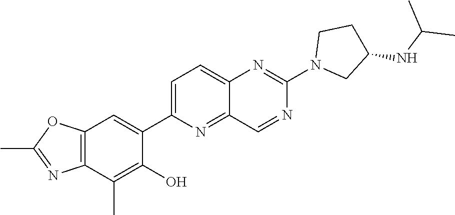 [SMILES: Cc1nc2c(C)c(O)c(-c3ccc4nc(N5CC[C@H](NC(C)C)C5)ncc4n3)cc2o1] |
| 417 | 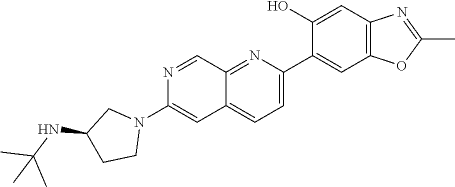 [SMILES: Cc1nc2cc(O)c(-c3ccc4cc(N5CC[C@@H](NC(C)(C)C)C5)ncc4n3)cc2o1] |
| 418 | 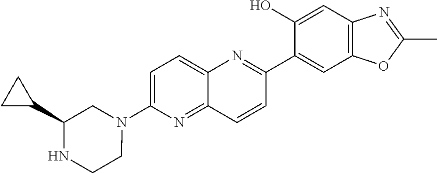 [SMILES: Cc1nc2cc(O)c(-c3ccc4nc(N5CCN[C@@H](C6CC6)C5)ccc4n3)cc2o1] |
| 419 | 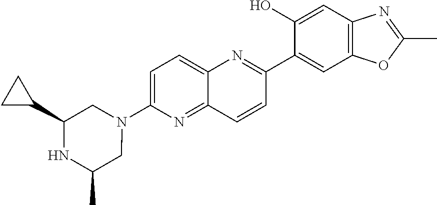 [SMILES: Cc1nc2cc(O)c(-c3ccc4nc(N5C[C@@H](C)N[C@@H](C6CC6)C5)ccc4n3)cc2o1] |
| 420 | 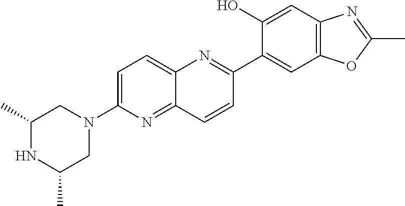 |
| 421 | 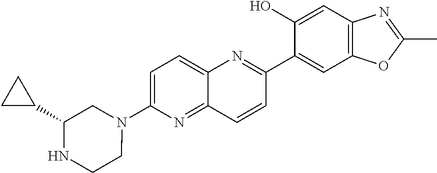 [SMILES: Cc1nc2cc(O)c(-c3ccc4nc(N5CCN[C@H](C6CC6)C5)ccc4n3)cc2o1] |
| 422 | 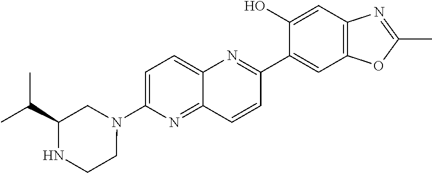 [SMILES: Cc1nc2cc(O)c(-c3ccc4nc(N5CCN[C@@H](C(C)C)C5)ccc4n3)cc2o1] |
| 423 | 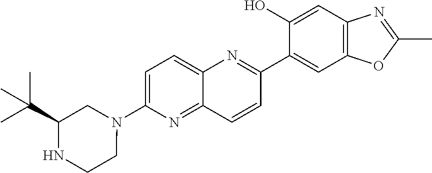 [SMILES: Cc1nc2cc(O)c(-c3ccc4nc(N5CCN[C@@H](C(C)(C)C)C5)ccc4n3)cc2o1] |
| 424 | 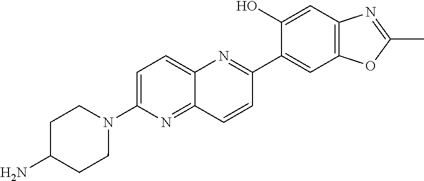 [SMILES: Cc1nc2cc(O)c(-c3ccc4nc(N5CCC(N)CC5)ccc4n3)cc2o1] |
| 425 | 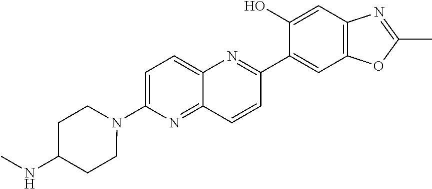 |
| 426 | 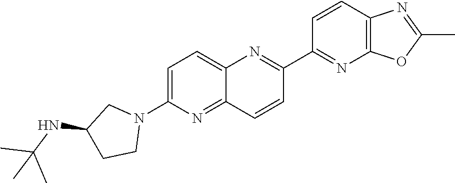 [SMILES: Cc1nc2ccc(-c3ccc4nc(N5CC[C@@H](NC(C)(C)C)C5)ccc4n3)nc2o1] |
| 427 | 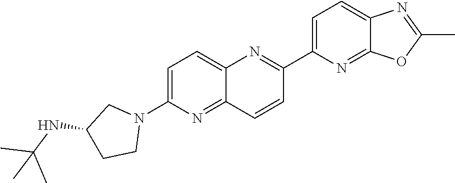 [SMILES: Cc1nc2ccc(-c3ccc4nc(N5CC[C@H](NC(C)(C)C)C5)ccc4n3)nc2o1] |
| 428 | 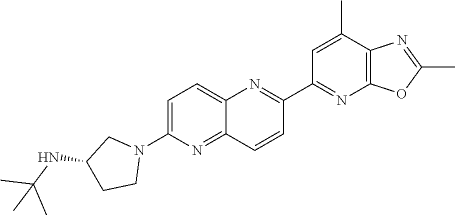 [SMILES: Cc1nc2c(C)cc(-c3ccc4nc(N5CC[C@H](NC(C)(C)C)C5)ccc4n3)nc2o1] |
| 429 | 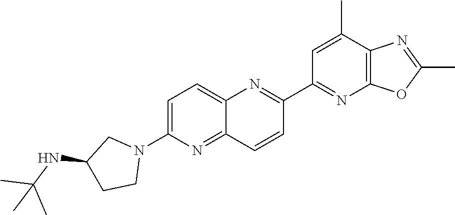 [SMILES: Cc1nc2c(C)cc(-c3ccc4nc(N5CC[C@@H](NC(C)(C)C)C5)ccc4n3)nc2o1] |
| 430 | 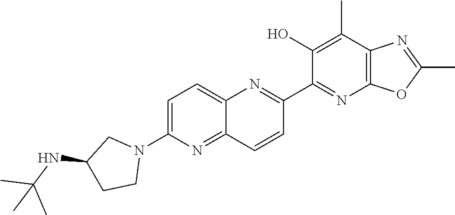 [SMILES: Cc1nc2c(C)c(O)c(-c3ccc4nc(N5CC[C@@H](NC(C)(C)C)C5)ccc4n3)nc2o1] |
| 431 | 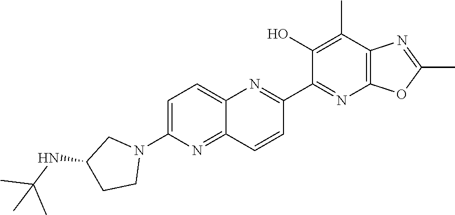 [SMILES: Cc1nc2c(C)c(O)c(-c3ccc4nc(N5CC[C@H](NC(C)(C)C)C5)ccc4n3)nc2o1] |
| 432 | 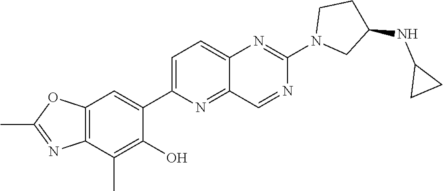 [SMILES: Cc1nc2c(C)c(O)c(-c3ccc4nc(N5CC[C@@H](NC6CC6)C5)ncc4n3)cc2o1] |
| 433 | 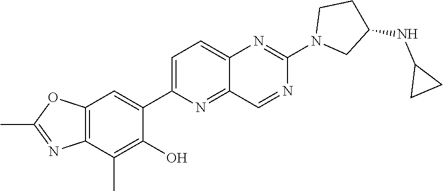 [SMILES: Cc1nc2c(C)c(O)c(-c3ccc4nc(N5CC[C@H](NC6CC6)C5)ncc4n3)cc2o1] |
| 434 | 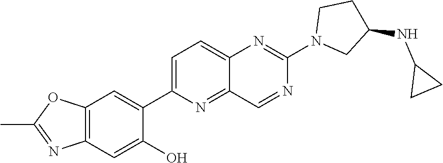 [SMILES: Cc1nc2cc(O)c(-c3ccc4nc(N5CC[C@@H](NC6CC6)C5)ncc4n3)cc2o1] |
| 435 | 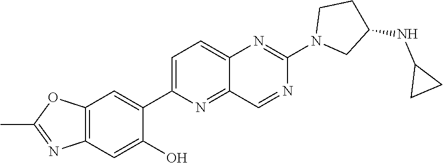 [SMILES: Cc1nc2cc(O)c(-c3ccc4nc(N5CC[C@H](NC6CC6)C5)ncc4n3)cc2o1] |
| 436 | 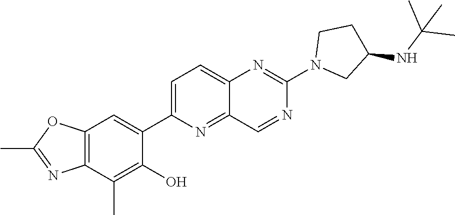 [SMILES: Cc1nc2c(C)c(O)c(-c3ccc4nc(N5CC[C@@H](NC(C)(C)C)C5)ncc4n3)cc2o1] |
| 437 | 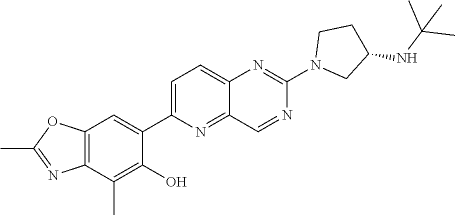 [SMILES: Cc1nc2c(C)c(O)c(-c3ccc4nc(N5CC[C@H](NC(C)(C)C)C5)ncc4n3)cc2o1] |
| 438 | 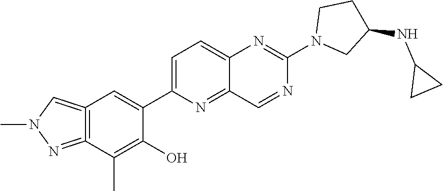 |
| 439 | 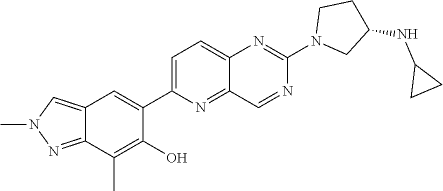 [SMILES: Cc1c(O)c(-c2ccc3nc(N4CC[C@H](NC5CC5)C4)ncc3n2)cc2cn(C)nc12] |
| 440 | 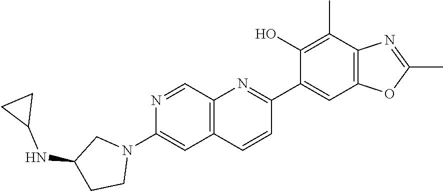 [SMILES: Cc1nc2c(C)c(O)c(-c3ccc4cc(N5CC[C@@H](NC6CC6)C5)ncc4n3)cc2o1] |
| 441 | 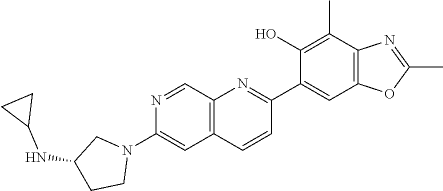 [SMILES: Cc1nc2c(C)c(O)c(-c3ccc4cc(N5CC[C@H](NC6CC6)C5)ncc4n3)cc2o1] |
| 442 | 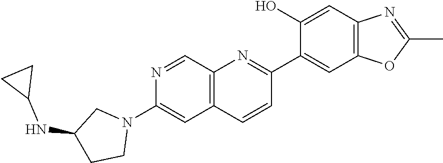 [SMILES: Cc1nc2cc(O)c(-c3ccc4cc(N5CC[C@@H](NC6CC6)C5)ncc4n3)cc2o1] |
| 443 | 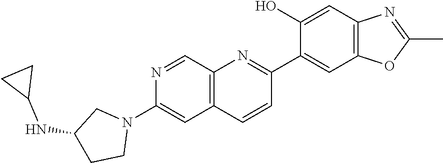 [SMILES: Cc1nc2cc(O)c(-c3ccc4cc(N5CC[C@H](NC6CC6)C5)ncc4n3)cc2o1] |
| 444 | 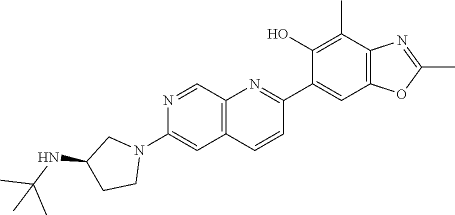 [SMILES: Cc1nc2c(C)c(O)c(-c3ccc4cc(N5CC[C@@H](NC(C)(C)C)C5)ncc4n3)cc2o1] |
| 445 | 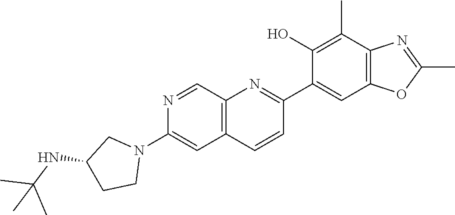 [SMILES: Cc1nc2c(C)c(O)c(-c3ccc4cc(N5CC[C@H](NC(C)(C)C)C5)ncc4n3)cc2o1] |
| 446 | 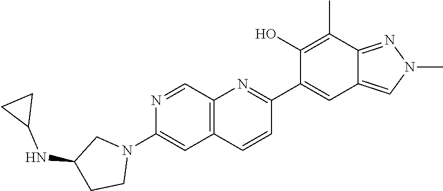 [SMILES: Cc1c(O)c(-c2ccc3cc(N4CC[C@@H](NC5CC5)C4)ncc3n2)cc2cn(C)nc12] |
| 447 | 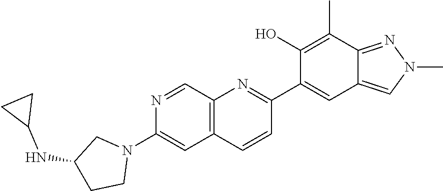 [SMILES: Cc1c(O)c(-c2ccc3cc(N4CC[C@H](NC5CC5)C4)ncc3n2)cc2cn(C)nc12] |
| 448 | 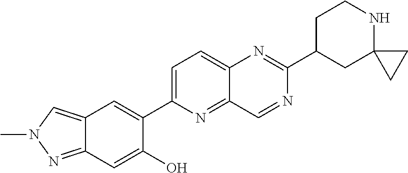 [SMILES: Cn1cc2cc(-c3ccc4nc(C5CCNC6(CC6)C5)ncc4n3)c(O)cc2n1] |
| 449 | 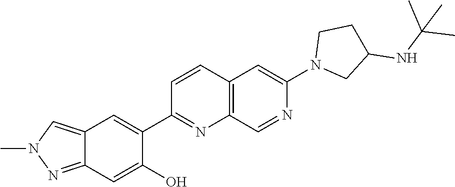 [SMILES: Cn1cc2cc(-c3ccc4cc(N5CCC(NC(C)(C)C)C5)ncc4n3)c(O)cc2n1] |
| 450 | 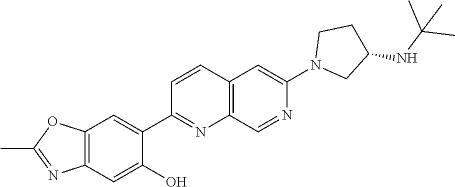 [SMILES: Cc1nc2cc(O)c(-c3ccc4cc(N5CC[C@H](NC(C)(C)C)C5)ncc4n3)cc2o1] |
| 451 | 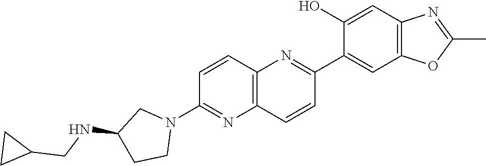 [SMILES: Cc1nc2cc(O)c(-c3ccc4nc(N5CC[C@@H](NCC6CC6)C5)ccc4n3)cc2o1] |
| 452 | 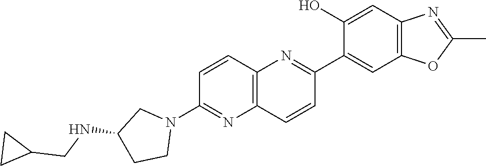 [SMILES: Cc1nc2cc(O)c(-c3ccc4nc(N5CC[C@H](NCC6CC6)C5)ccc4n3)cc2o1] |
| 453 | 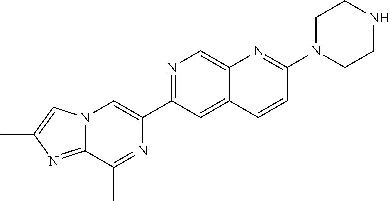 [SMILES: Cc1cn2cc(-c3cc4ccc(N5CCNCC5)nc4cn3)nc(C)c2n1] |
| 454 | 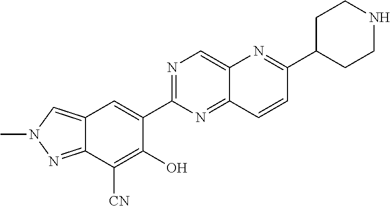 [SMILES: Cn1cc2cc(-c3ncc4nc(C5CCNCC5)ccc4n3)c(O)c(C#N)c2n1] |
| 455 | 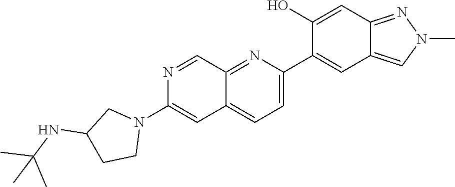 [SMILES: Cn1cc2cc(-c3ccc4cc(N5CCC(NC(C)(C)C)C5)ncc4n3)c(O)cc2n1] |
| 456 | 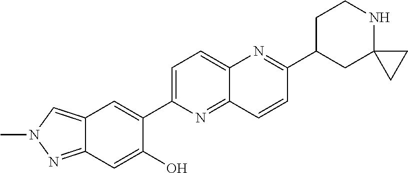 [SMILES: Cn1cc2cc(-c3ccc4nc(C5CCNC6(CC6)C5)ccc4n3)c(O)cc2n1] |
| 457 | 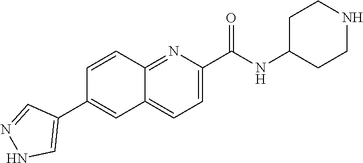 [SMILES: O=C(NC1CCNCC1)c1ccc2cc(-c3cn[nH]c3)ccc2n1] |
| 458 | 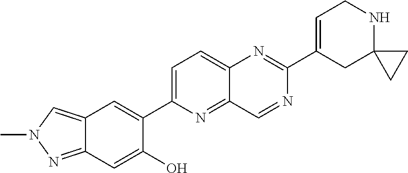 [SMILES: Cn1cc2cc(-c3ccc4nc(C5=CCNC6(CC6)C5)ncc4n3)c(O)cc2n1] |
| 459 | 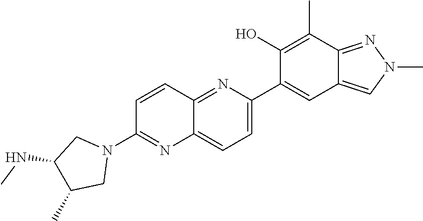 |
| 460 | 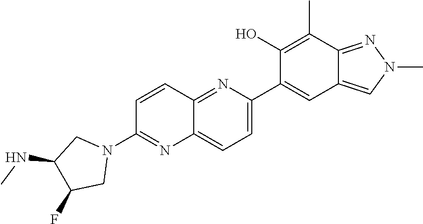 |
| 461 | 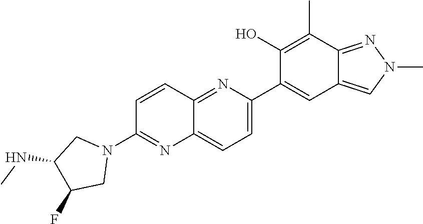 |
| 462 | 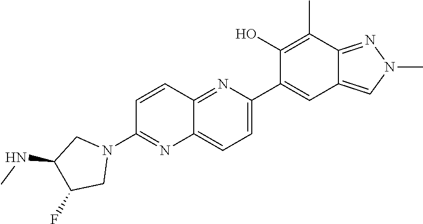 |
| 463 | 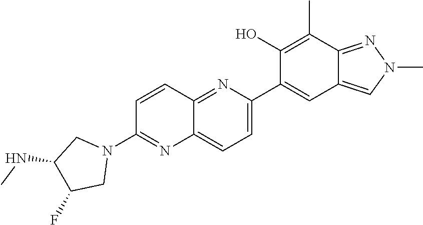 |
| 464 | 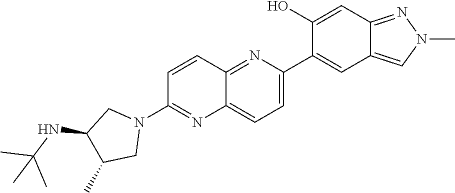 [SMILES: C[C@H]1CN(c2ccc3nc(-c4cc5cn(C)nc5cc4O)ccc3n2)C[C@@H]1NC(C)(C)C] |
| 465 | 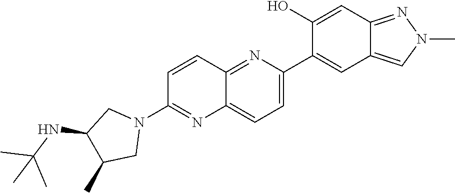 [SMILES: C[C@@H]1CN(c2ccc3nc(-c4cc5cn(C)nc5cc4O)ccc3n2)C[C@@H]1NC(C)(C)C] |
| 466 | 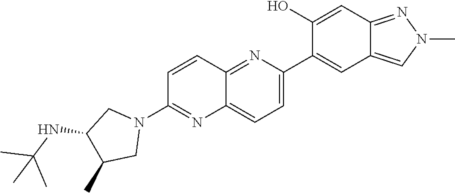 [SMILES: C[C@@H]1CN(c2ccc3nc(-c4cc5cn(C)nc5cc4O)ccc3n2)C[C@H]1NC(C)(C)C] |
| 467 | 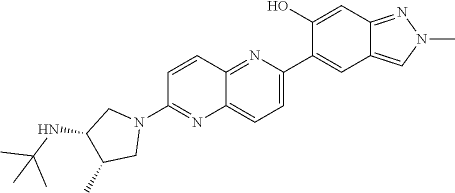 [SMILES: C[C@H]1CN(c2ccc3nc(-c4cc5cn(C)nc5cc4O)ccc3n2)C[C@H]1NC(C)(C)C] |
| 468 | 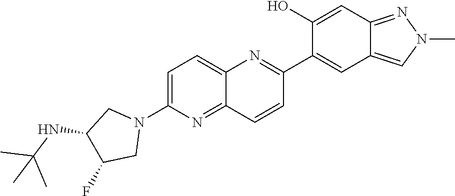 [SMILES: Cn1cc2cc(-c3ccc4nc(N5C[C@H](F)[C@H](NC(C)(C)C)C5)ccc4n3)c(O)cc2n1] |
| 469 | 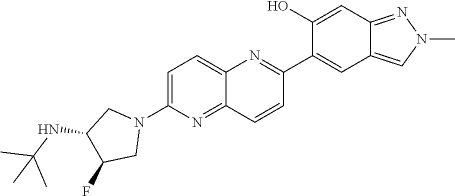 [SMILES: Cn1cc2cc(-c3ccc4nc(N5C[C@@H](F)[C@H](NC(C)(C)C)C5)ccc4n3)c(O)cc2n1] |
| 470 | 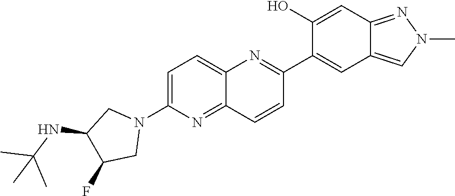 [SMILES: Cn1cc2cc(-c3ccc4nc(N5C[C@@H](F)[C@@H](NC(C)(C)C)C5)ccc4n3)c(O)cc2n1] |
| 471 | 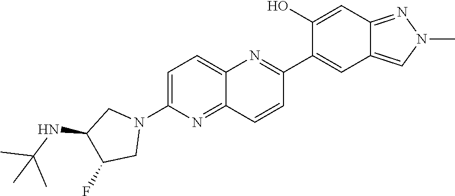 [SMILES: Cn1cc2cc(-c3ccc4nc(N5C[C@H](NC(C)(C)C)[C@@H](F)C5)ccc4n3)c(O)cc2n1] |
| 472 | 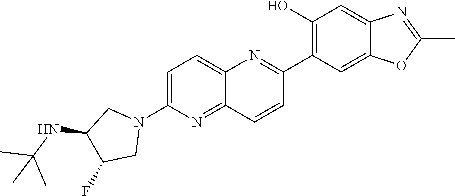 [SMILES: Cc1nc2cc(O)c(-c3ccc4nc(N5C[C@H](NC(C)(C)C)[C@@H](F)C5)ccc4n3)cc2o1] |
| 473 | 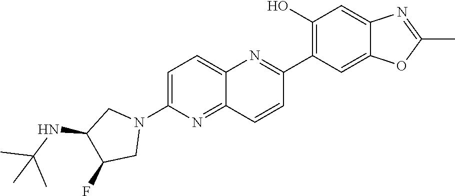 [SMILES: Cc1nc2cc(O)c(-c3ccc4nc(N5C[C@@H](F)[C@@H](NC(C)(C)C)C5)ccc4n3)cc2o1] |
| 474 | 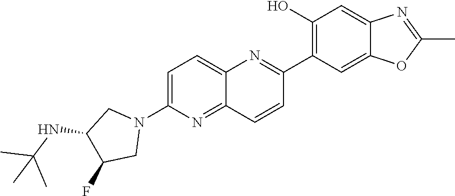 [SMILES: Cc1nc2cc(O)c(-c3ccc4nc(N5C[C@@H](F)[C@H](NC(C)(C)C)C5)ccc4n3)cc2o1] |
| 475 | 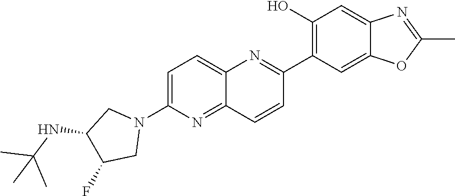 [SMILES: Cc1nc2cc(O)c(-c3ccc4nc(N5C[C@H](F)[C@H](NC(C)(C)C)C5)ccc4n3)cc2o1] |
| 476 | 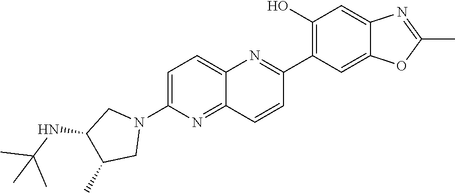 [SMILES: Cc1nc2cc(O)c(-c3ccc4nc(N5C[C@H](C)[C@H](NC(C)(C)C)C5)ccc4n3)cc2o1] |
| 477 | 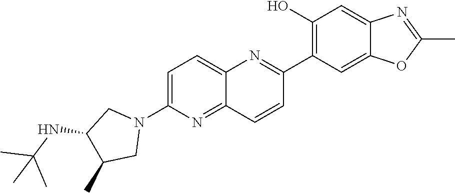 [SMILES: Cc1nc2cc(O)c(-c3ccc4nc(N5C[C@@H](C)[C@H](NC(C)(C)C)C5)ccc4n3)cc2o1] |
| 478 | 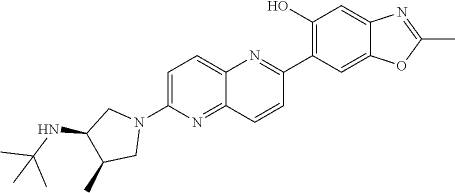 [SMILES: Cc1nc2cc(O)c(-c3ccc4nc(N5C[C@@H](C)[C@@H](NC(C)(C)C)C5)ccc4n3)cc2o1] |
| 479 | 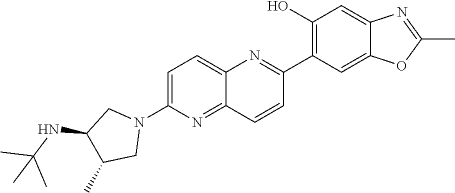 [SMILES: Cc1nc2cc(O)c(-c3ccc4nc(N5C[C@H](NC(C)(C)C)[C@@H](C)C5)ccc4n3)cc2o1] |
| 480 | 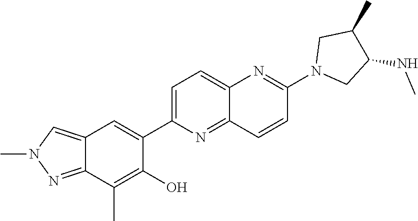 |
| 481 | 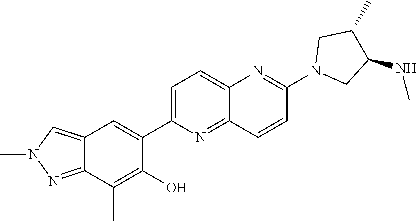 |
| 482 | 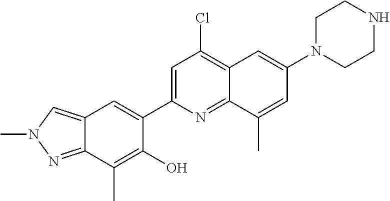 [SMILES: Cc1c(O)c(-c2cc(Cl)c3cc(N4CCNCC4)cc(C)c3n2)cc2cn(C)nc12] |
| 483 | 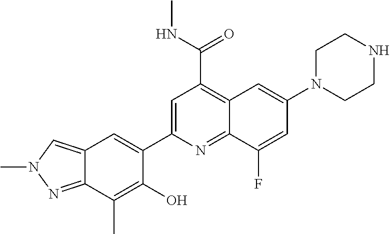 [SMILES: CNC(=O)c1cc(-c2cc3cn(C)nc3c(C)c2O)nc2c(F)cc(N3CCNCC3)cc12] |
| 484 | 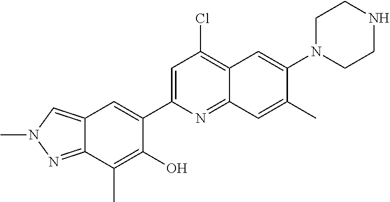 [SMILES: Cc1cc2nc(-c3cc4cn(C)nc4c(C)c3O)cc(Cl)c2cc1N1CCNCC1] |
| 485 | 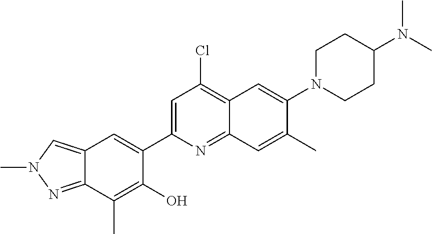 |
| 486 | 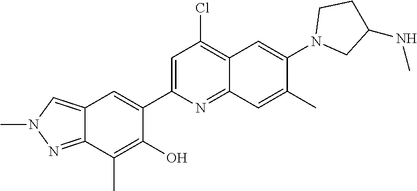 |
| 487 | 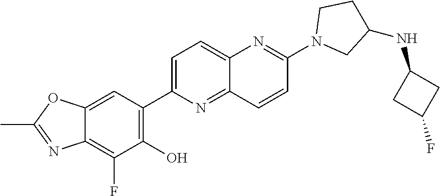 [SMILES: Cc1nc2c(F)c(O)c(-c3ccc4nc(N5CCC(N[C@H]6C[C@H](F)C6)C5)ccc4n3)cc2o1] |
| 488 | 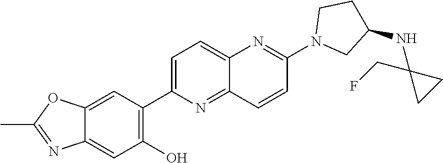 [SMILES: Cc1nc2cc(O)c(-c3ccc4nc(N5CC[C@@H](NC6(CF)CC6)C5)ccc4n3)cc2o1] |
| 489 | 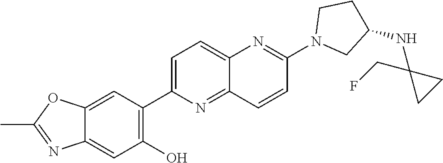 [SMILES: Cc1nc2cc(O)c(-c3ccc4nc(N5CC[C@H](NC6(CF)CC6)C5)ccc4n3)cc2o1] |
| 490 | 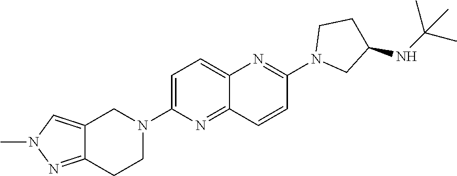 [SMILES: Cn1cc2c(n1)CCN(c1ccc3nc(N4CC[C@@H](NC(C)(C)C)C4)ccc3n1)C2] |
| 491 | 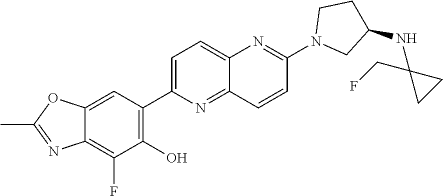 [SMILES: Cc1nc2c(F)c(O)c(-c3ccc4nc(N5CC[C@@H](NC6(CF)CC6)C5)ccc4n3)cc2o1] |
| 492 | 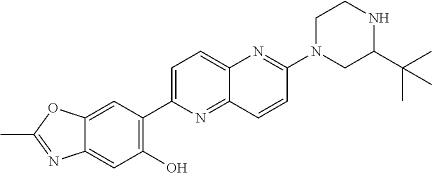 [SMILES: Cc1nc2cc(O)c(-c3ccc4nc(N5CCNC(C(C)(C)C)C5)ccc4n3)cc2o1] |
| 493 | 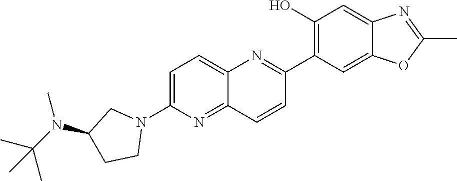 [SMILES: Cc1nc2cc(O)c(-c3ccc4nc(N5CC[C@@H](N(C)C(C)(C)C)C5)ccc4n3)cc2o1] |
| 494 | 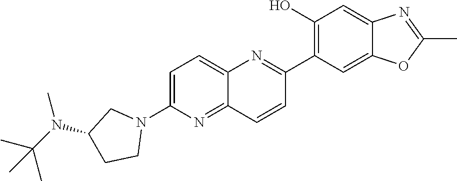 [SMILES: Cc1nc2cc(O)c(-c3ccc4nc(N5CC[C@H](N(C)C(C)(C)C)C5)ccc4n3)cc2o1] |
| 495 | 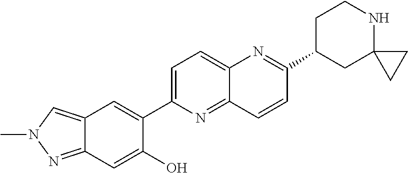 [SMILES: Cn1cc2cc(-c3ccc4nc([C@@H]5CCNC6(CC6)C5)ccc4n3)c(O)cc2n1] |
| 496 | 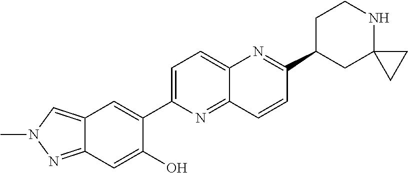 [SMILES: Cn1cc2cc(-c3ccc4nc([C@H]5CCNC6(CC6)C5)ccc4n3)c(O)cc2n1] |
| 497 | 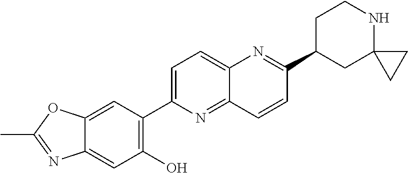 [SMILES: Cc1nc2cc(O)c(-c3ccc4nc([C@H]5CCNC6(CC6)C5)ccc4n3)cc2o1] |
| 498 | 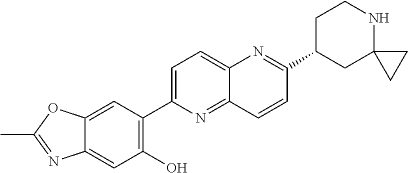 [SMILES: Cc1nc2cc(O)c(-c3ccc4nc([C@@H]5CCNC6(CC6)C5)ccc4n3)cc2o1] |
| 499 | 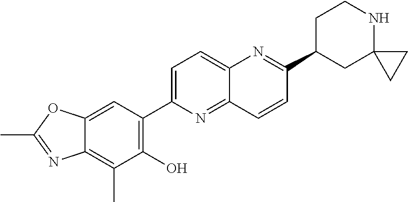 [SMILES: Cc1nc2c(C)c(O)c(-c3ccc4nc([C@H]5CCNC6(CC6)C5)ccc4n3)cc2o1] |
| 500 | 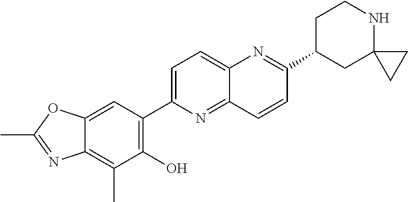 [SMILES: Cc1nc2c(C)c(O)c(-c3ccc4nc([C@@H]5CCNC6(CC6)C5)ccc4n3)cc2o1] |
| 501 | 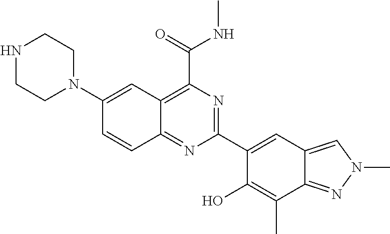 [SMILES: CNC(=O)c1nc(-c2cc3cn(C)nc3c(C)c2O)nc2ccc(N3CCNCC3)cc12] |
| 502 | 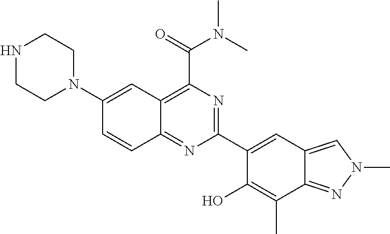 [SMILES: Cc1c(O)c(-c2nc(C(=O)N(C)C)c3cc(N4CCNCC4)ccc3n2)cc2cn(C)nc12] |
| 503 | 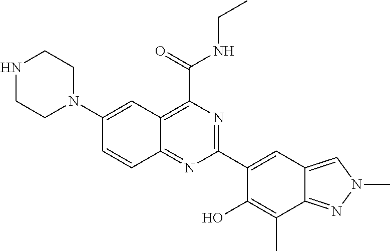 [SMILES: CCNC(=O)c1nc(-c2cc3cn(C)nc3c(C)c2O)nc2ccc(N3CCNCC3)cc12] |
| 504 | 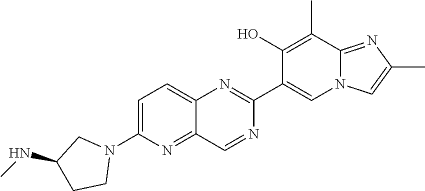 |
| 505 | 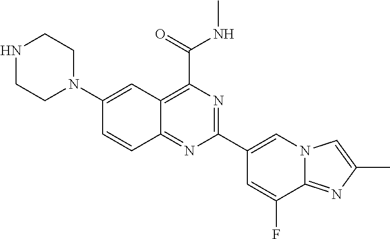 [SMILES: CNC(=O)c1nc(-c2cc(F)c3nc(C)cn3c2)nc2ccc(N3CCNCC3)cc12] |
| 506 | 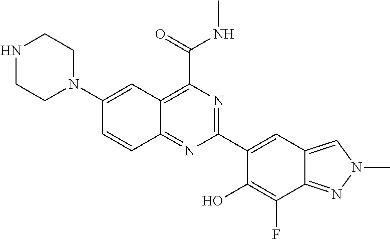 [SMILES: CNC(=O)c1nc(-c2cc3cn(C)nc3c(F)c2O)nc2ccc(N3CCNCC3)cc12] |
| 507 | 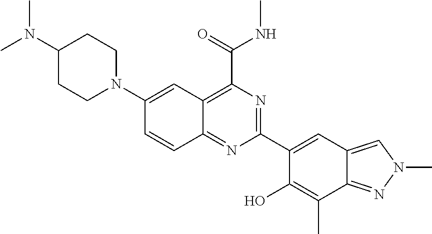 |
| 508 | 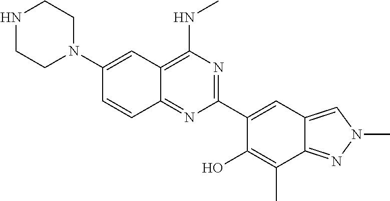 [SMILES: CNc1nc(-c2cc3cn(C)nc3c(C)c2O)nc2ccc(N3CCNCC3)cc12] |
| 509 | 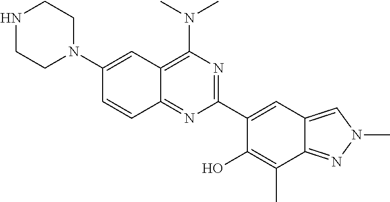 [SMILES: Cc1c(O)c(-c2nc(N(C)C)c3cc(N4CCNCC4)ccc3n2)cc2cn(C)nc12] |
| 510 | 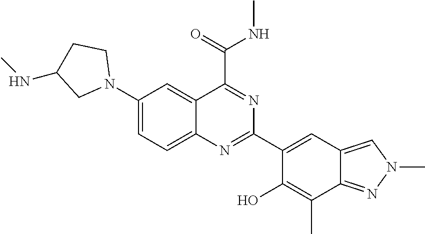 |
| 511 | 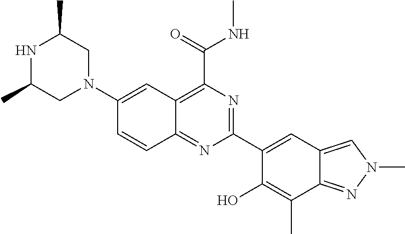 |
| 512 | 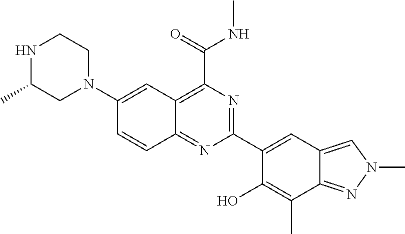 |
| 513 | 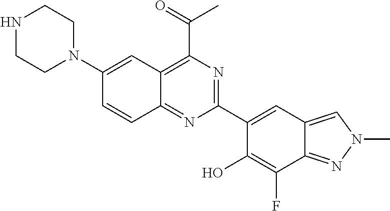 [SMILES: CC(=O)c1nc(-c2cc3cn(C)nc3c(F)c2O)nc2ccc(N3CCNCC3)cc12] |
| 514 | 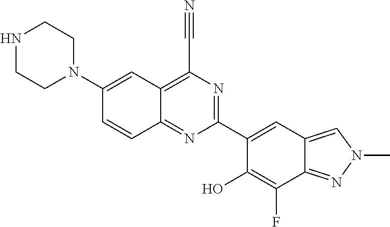 [SMILES: Cn1cc2cc(-c3nc(C#N)c4cc(N5CCNCC5)ccc4n3)c(O)c(F)c2n1] |
| 515 | 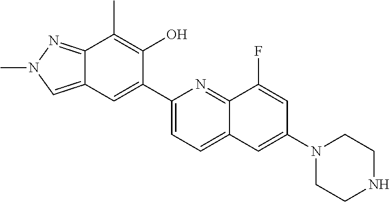 [SMILES: Cc1c(O)c(-c2ccc3cc(N4CCNCC4)cc(F)c3n2)cc2cn(C)nc12] |
| 516 | 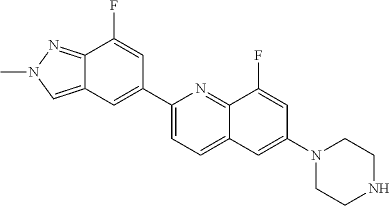 [SMILES: Cn1cc2cc(-c3ccc4cc(N5CCNCC5)cc(F)c4n3)cc(F)c2n1] |
| 517 | 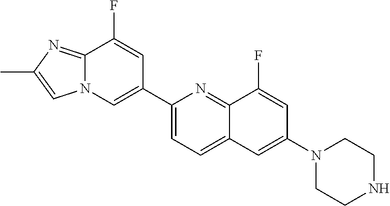 [SMILES: Cc1cn2cc(-c3ccc4cc(N5CCNCC5)cc(F)c4n3)cc(F)c2n1] |
| 518 | 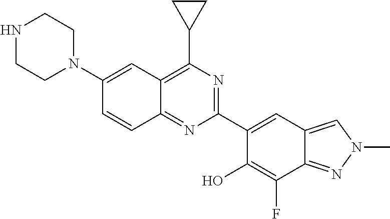 [SMILES: Cn1cc2cc(-c3nc(C4CC4)c4cc(N5CCNCC5)ccc4n3)c(O)c(F)c2n1] |
| 519 | 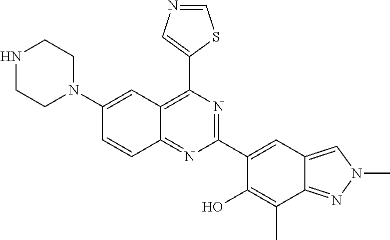 [SMILES: Cc1c(O)c(-c2nc(-c3cncs3)c3cc(N4CCNCC4)ccc3n2)cc2cn(C)nc12] |
| 520 | 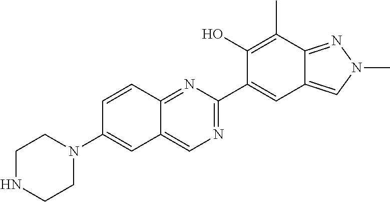 [SMILES: Cc1c(O)c(-c2ncc3cc(N4CCNCC4)ccc3n2)cc2cn(C)nc12] |
| 521 | 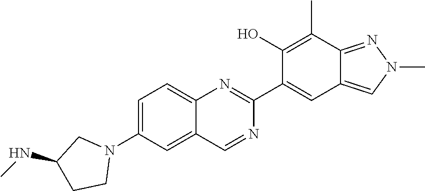 |
| 522 | 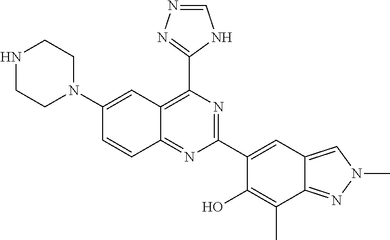 [SMILES: Cc1c(O)c(-c2nc(-c3nnc[nH]3)c3cc(N4CCNCC4)ccc3n2)cc2cn(C)nc12] |
| 523 | 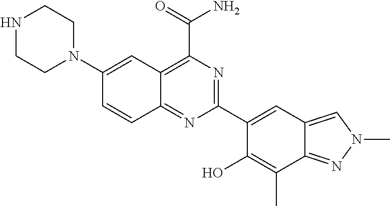 [SMILES: Cc1c(O)c(-c2nc(C(N)=O)c3cc(N4CCNCC4)ccc3n2)cc2cn(C)nc12] |
| 524 | 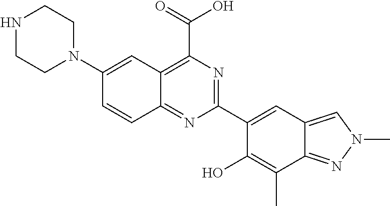 [SMILES: Cc1c(O)c(-c2nc(C(=O)O)c3cc(N4CCNCC4)ccc3n2)cc2cn(C)nc12] |
| 525 | 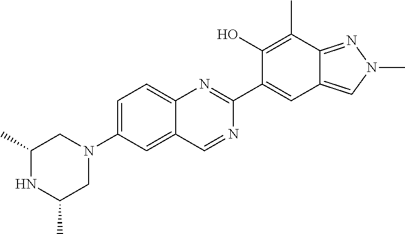 |
| 526 | 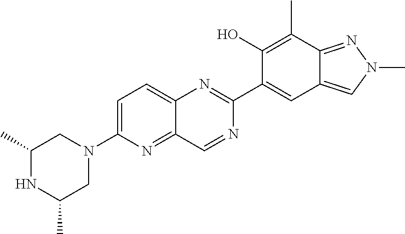 |
| 527 | 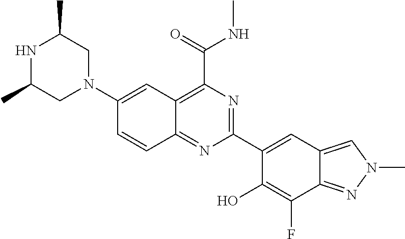 |
| 528 | 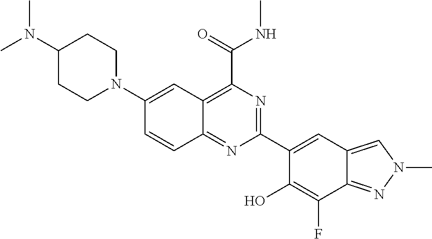 |
| 529 | 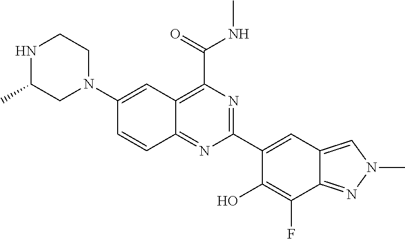 |
| 530 | 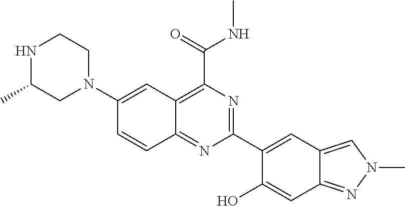 |
| 531 | 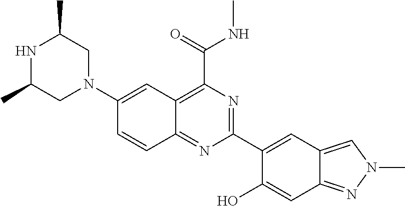 |
| 532 | 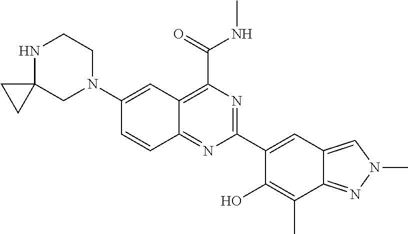 [SMILES: CNC(=O)c1nc(-c2cc3cn(C)nc3c(C)c2O)nc2ccc(N3CCNC4(CC4)C3)cc12] |
| 533 | 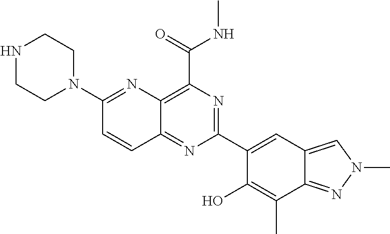 [SMILES: CNC(=O)c1nc(-c2cc3cn(C)nc3c(C)c2O)nc2ccc(N3CCNCC3)nc12] |
| 534 | 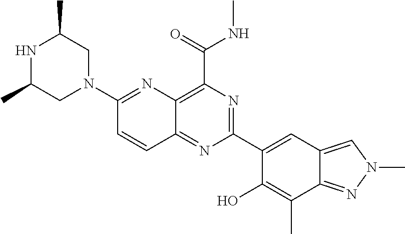 |
| 535 | 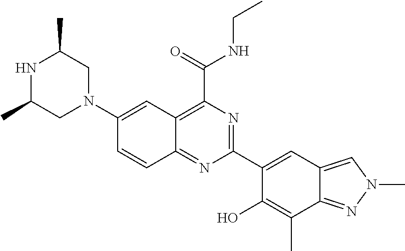 |
| 536 | 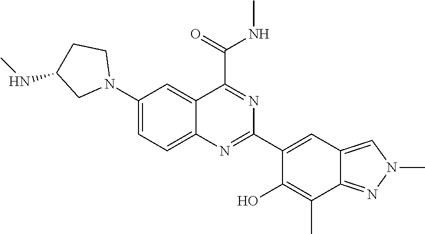 |
| 537 | 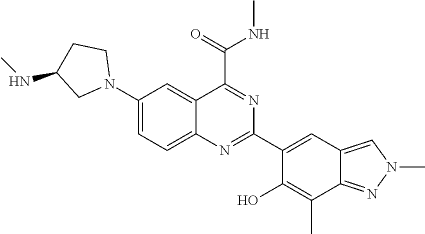 |
| 538 | 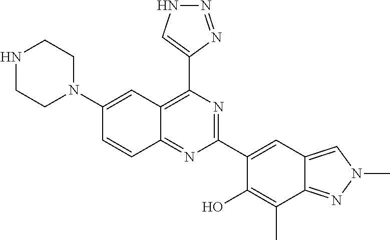 [SMILES: Cc1c(O)c(-c2nc(-c3c[nH]nn3)c3cc(N4CCNCC4)ccc3n2)cc2cn(C)nc12] |
| 539 | 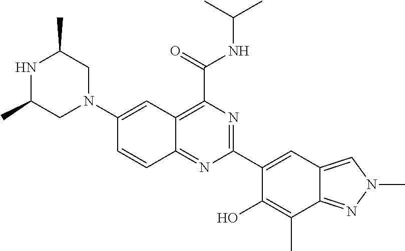 |
| 540 | 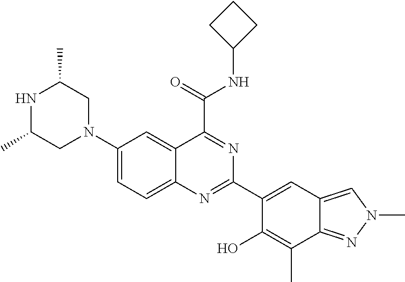 |
| 541 | 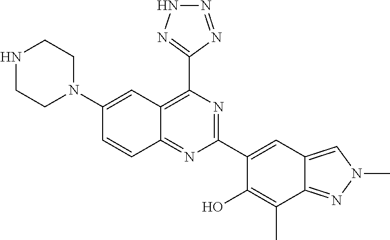 [SMILES: Cc1c(O)c(-c2nc(-c3nn[nH]n3)c3cc(N4CCNCC4)ccc3n2)cc2cn(C)nc12] |
| 542 | 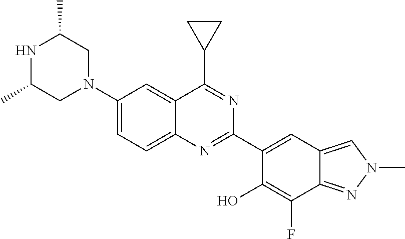 |
| 543 | 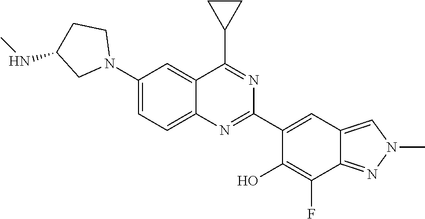 |
| 544 | 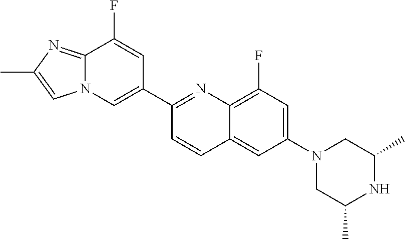 |
| 545 | 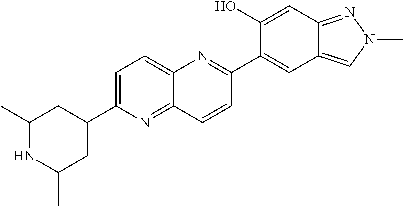 |
| 546 | 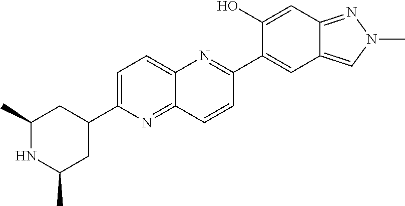 |
| 547 | 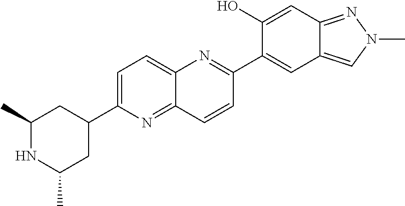 |
| 548 | 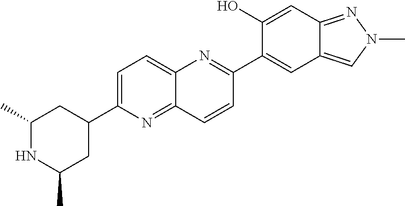 |
| 549 | 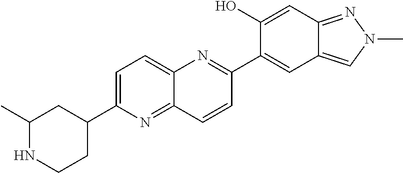 |
| 550 | 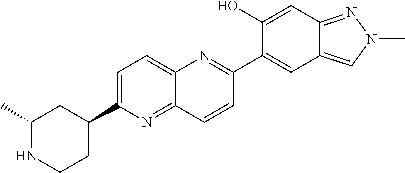 |
| 551 | 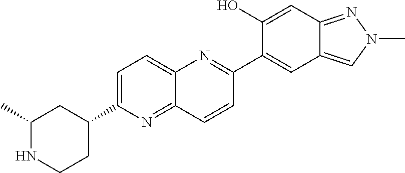 |
| 552 | 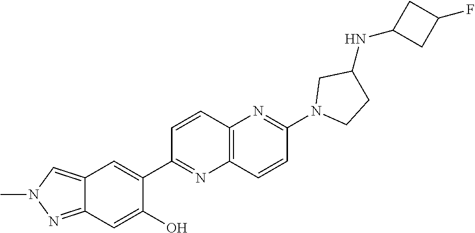 [SMILES: Cn1cc2cc(-c3ccc4nc(N5CCC(NC6CC(F)C6)C5)ccc4n3)c(O)cc2n1] |
| 553 | 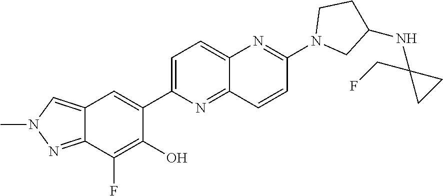 [SMILES: Cn1cc2cc(-c3ccc4nc(N5CCC(NC6(CF)CC6)C5)ccc4n3)c(O)c(F)c2n1] |
| 554 | 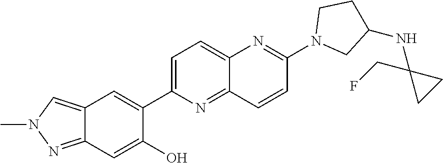 [SMILES: Cn1cc2cc(-c3ccc4nc(N5CCC(NC6(CF)CC6)C5)ccc4n3)c(O)cc2n1] |
| 555 | 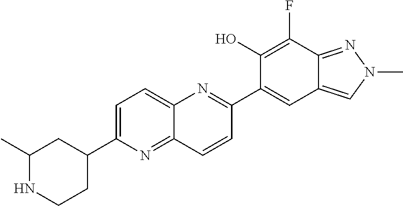 |
| 556 | 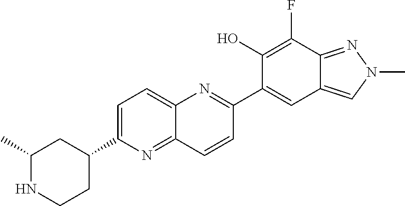 |
| 557 | 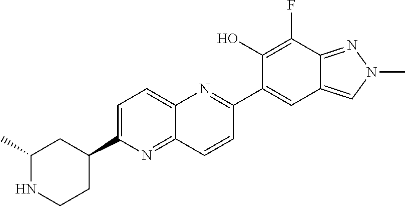 |
| 558 | 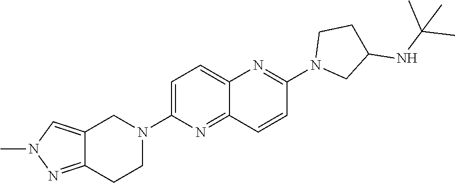 [SMILES: Cn1cc2c(n1)CCN(c1ccc3nc(N4CCC(NC(C)(C)C)C4)ccc3n1)C2] |
| 559 | 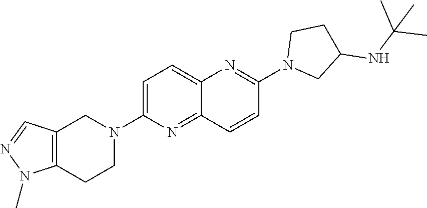 [SMILES: Cn1ncc2c1CCN(c1ccc3nc(N4CCC(NC(C)(C)C)C4)ccc3n1)C2] |
| 560 | 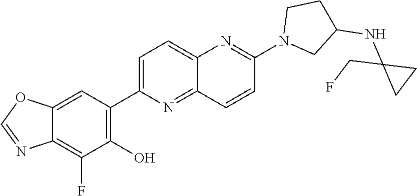 [SMILES: Oc1c(-c2ccc3nc(N4CCC(NC5(CF)CC5)C4)ccc3n2)cc2ocnc2c1F] |
| 561 | 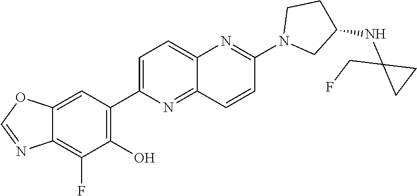 [SMILES: Oc1c(-c2ccc3nc(N4CC[C@H](NC5(CF)CC5)C4)ccc3n2)cc2ocnc2c1F] |
| 562 | 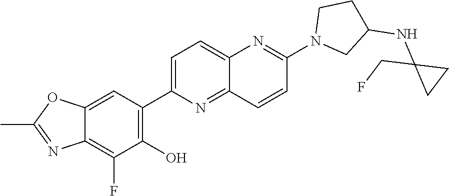 [SMILES: Cc1nc2c(F)c(O)c(-c3ccc4nc(N5CCC(NC6(CF)CC6)C5)ccc4n3)cc2o1] |
| 563 | 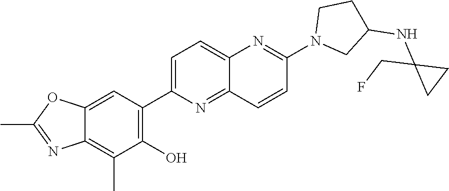 [SMILES: Cc1nc2c(C)c(O)c(-c3ccc4nc(N5CCC(NC6(CF)CC6)C5)ccc4n3)cc2o1] |
| 564 | 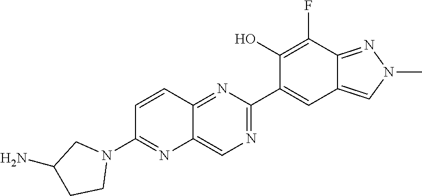 [SMILES: Cn1cc2cc(-c3ncc4nc(N5CCC(N)C5)ccc4n3)c(O)c(F)c2n1] |
| 565 | 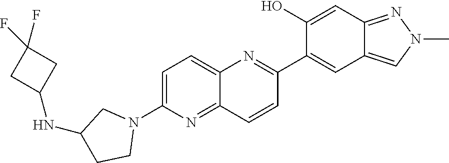 [SMILES: Cn1cc2cc(-c3ccc4nc(N5CCC(NC6CC(F)(F)C6)C5)ccc4n3)c(O)cc2n1] |
| 566 | 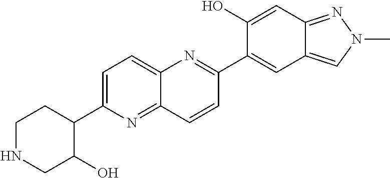 [SMILES: Cn1cc2cc(-c3ccc4nc(C5CCNCC5O)ccc4n3)c(O)cc2n1] |
| 567 | 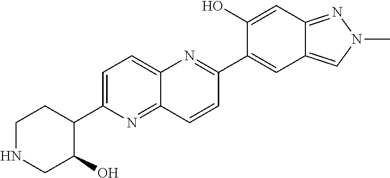 [SMILES: Cn1cc2cc(-c3ccc4nc(C5CCNC[C@@H]5O)ccc4n3)c(O)cc2n1] |
| 568 | 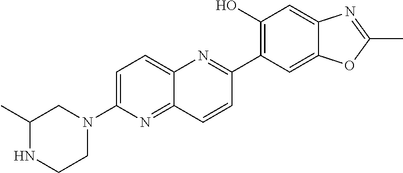 |
| 569 | 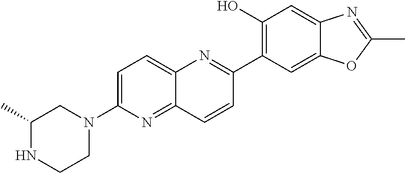 |
| 570 | 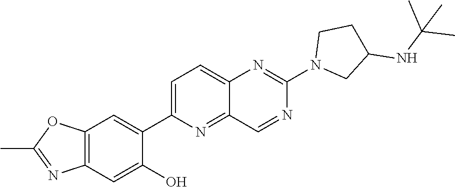 [SMILES: Cc1nc2cc(O)c(-c3ccc4nc(N5CCC(NC(C)(C)C)C5)ncc4n3)cc2o1] |
| 571 | 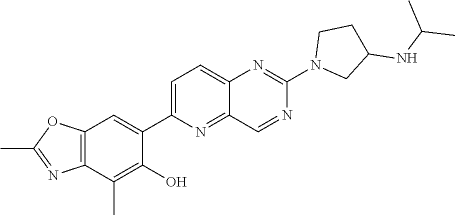 [SMILES: Cc1nc2c(C)c(O)c(-c3ccc4nc(N5CCC(NC(C)C)C5)ncc4n3)cc2o1] |
| 572 | 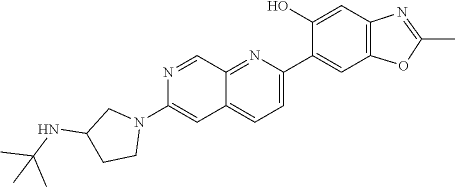 [SMILES: Cc1nc2cc(O)c(-c3ccc4cc(N5CCC(NC(C)(C)C)C5)ncc4n3)cc2o1] |
| 573 | 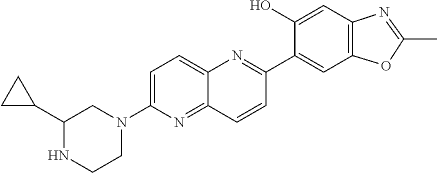 [SMILES: Cc1nc2cc(O)c(-c3ccc4nc(N5CCNC(C6CC6)C5)ccc4n3)cc2o1] |
| 574 | 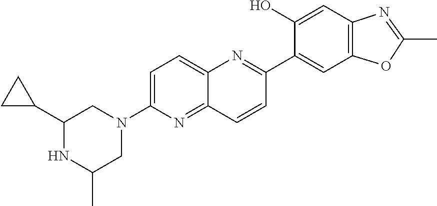 [SMILES: Cc1nc2cc(O)c(-c3ccc4nc(N5CC(C)NC(C6CC6)C5)ccc4n3)cc2o1] |
| 575 | 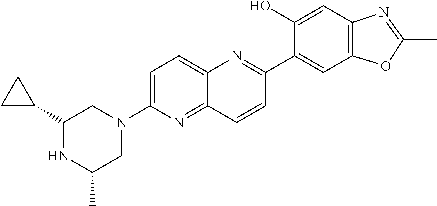 |
| 576 | 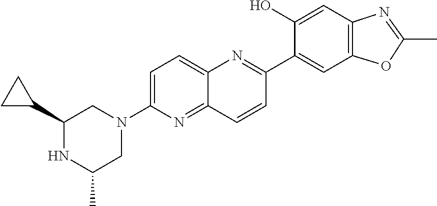 [SMILES: Cc1nc2cc(O)c(-c3ccc4nc(N5C[C@H](C)N[C@@H](C6CC6)C5)ccc4n3)cc2o1] |
| 577 | 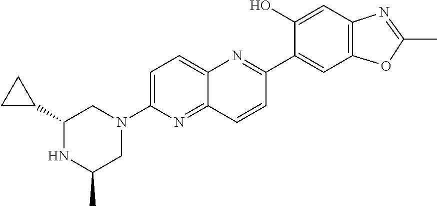 [SMILES: Cc1nc2cc(O)c(-c3ccc4nc(N5C[C@@H](C)N[C@H](C6CC6)C5)ccc4n3)cc2o1] |
| 578 | 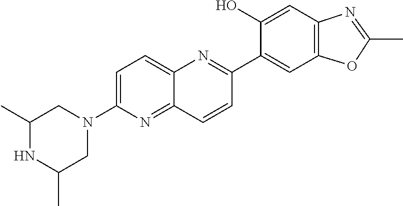 |
| 579 | 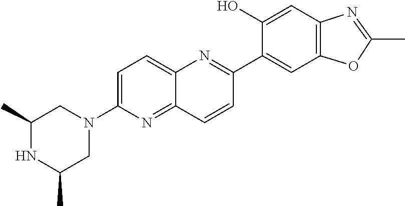 |
| 580 | 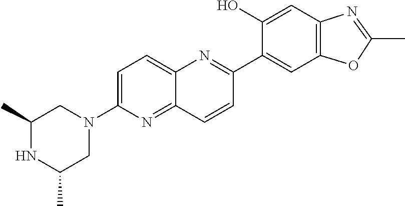 |
| 581 | 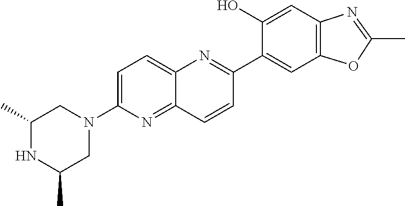 |
| 582 | 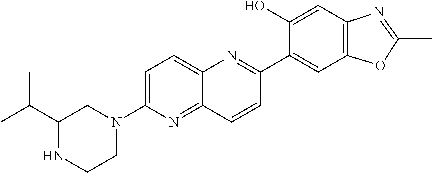 [SMILES: Cc1nc2cc(O)c(-c3ccc4nc(N5CCNC(C(C)C)C5)ccc4n3)cc2o1] |
| 583 | 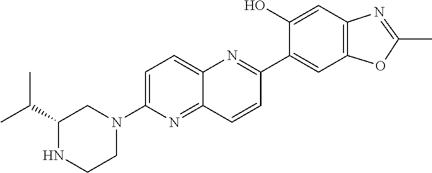 [SMILES: Cc1nc2cc(O)c(-c3ccc4nc(N5CCN[C@H](C(C)C)C5)ccc4n3)cc2o1] |
| 584 | 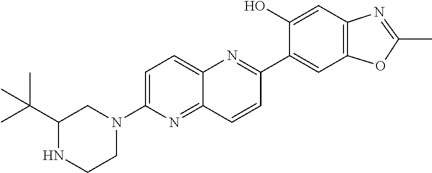 [SMILES: Cc1nc2cc(O)c(-c3ccc4nc(N5CCNC(C(C)(C)C)C5)ccc4n3)cc2o1] |
| 585 | 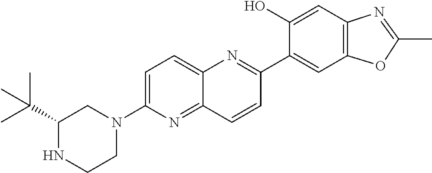 [SMILES: Cc1nc2cc(O)c(-c3ccc4nc(N5CCN[C@H](C(C)(C)C)C5)ccc4n3)cc2o1] |
| 586 | 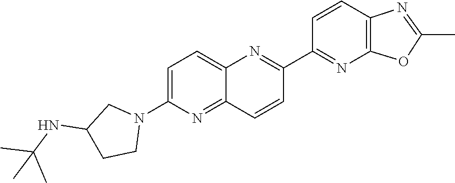 [SMILES: Cc1nc2ccc(-c3ccc4nc(N5CCC(NC(C)(C)C)C5)ccc4n3)nc2o1] |
| 587 | 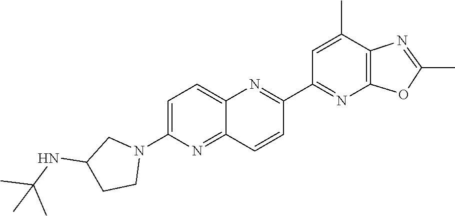 [SMILES: Cc1nc2c(C)cc(-c3ccc4nc(N5CCC(NC(C)(C)C)C5)ccc4n3)nc2o1] |
| 588 | 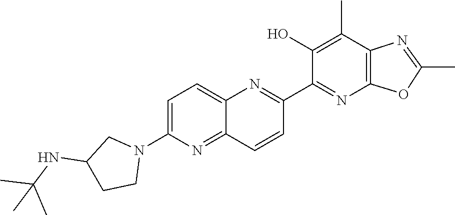 [SMILES: Cc1nc2c(C)c(O)c(-c3ccc4nc(N5CCC(NC(C)(C)C)C5)ccc4n3)nc2o1] |
| 589 | 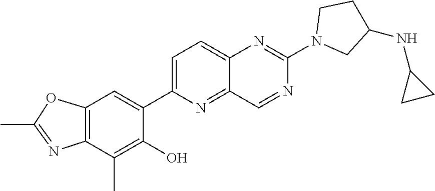 [SMILES: Cc1nc2c(C)c(O)c(-c3ccc4nc(N5CCC(NC6CC6)C5)ncc4n3)cc2o1] |
| 590 | 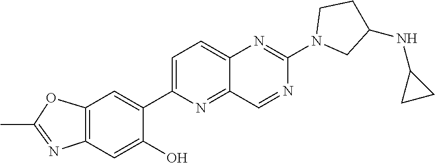 [SMILES: Cc1nc2cc(O)c(-c3ccc4nc(N5CCC(NC6CC6)C5)ncc4n3)cc2o1] |
| 591 | 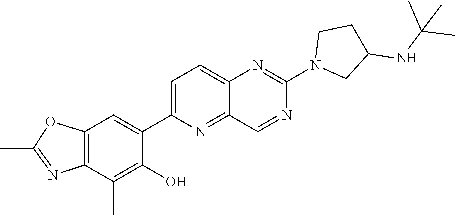 [SMILES: Cc1nc2c(C)c(O)c(-c3ccc4nc(N5CCC(NC(C)(C)C)C5)ncc4n3)cc2o1] |
| 592 | 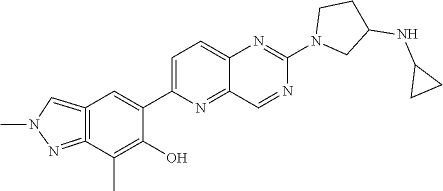 |
| 593 | 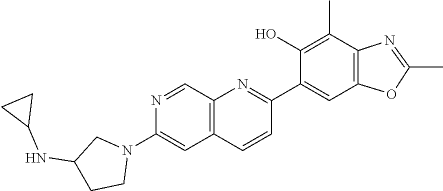 [SMILES: Cc1nc2c(C)c(O)c(-c3ccc4cc(N5CCC(NC6CC6)C5)ncc4n3)cc2o1] |
| 594 | 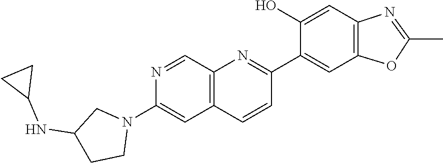 [SMILES: Cc1nc2cc(O)c(-c3ccc4cc(N5CCC(NC6CC6)C5)ncc4n3)cc2o1] |
| 595 | 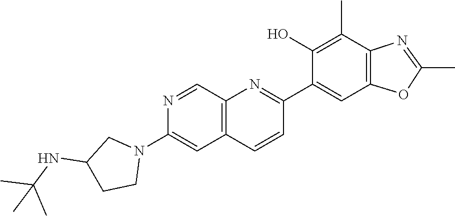 [SMILES: Cc1nc2c(C)c(O)c(-c3ccc4cc(N5CCC(NC(C)(C)C)C5)ncc4n3)cc2o1] |
| 596 | 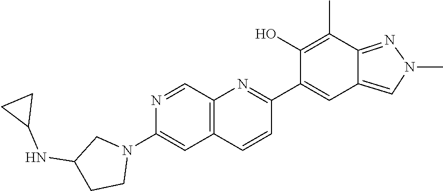 [SMILES: Cc1c(O)c(-c2ccc3cc(N4CCC(NC5CC5)C4)ncc3n2)cc2cn(C)nc12] |
| 597 | 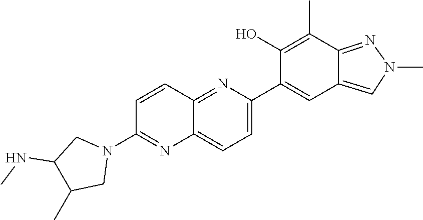 |
| 598 | 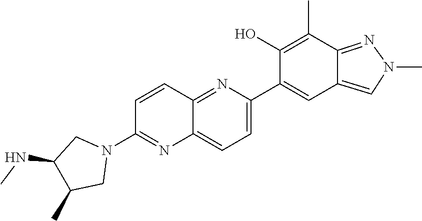 |
| 599 | 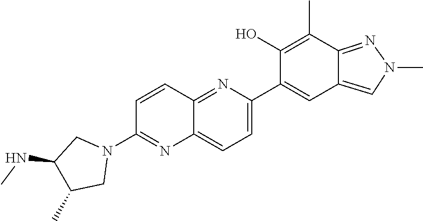 |
| 600 | 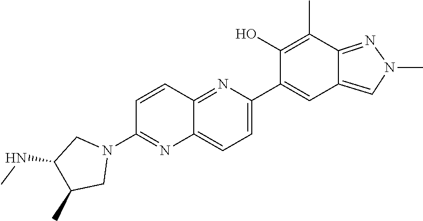 |
| 601 | 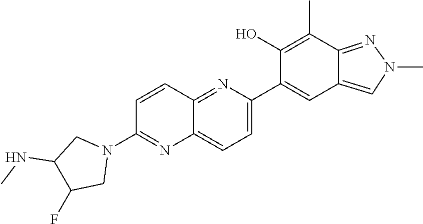 |
| 602 | 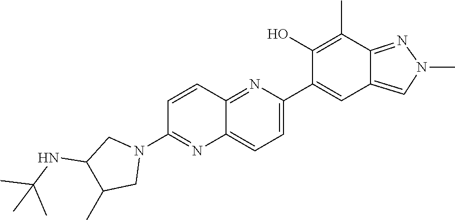 [SMILES: Cc1c(O)c(-c2ccc3nc(N4CC(C)C(NC(C)(C)C)C4)ccc3n2)cc2cn(C)nc12] |
| 603 | 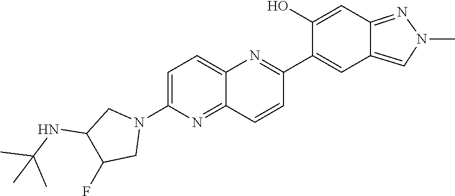 [SMILES: Cn1cc2cc(-c3ccc4nc(N5CC(F)C(NC(C)(C)C)C5)ccc4n3)c(O)cc2n1] |
| 604 | 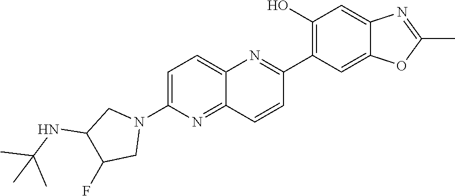 [SMILES: Cc1nc2cc(O)c(-c3ccc4nc(N5CC(F)C(NC(C)(C)C)C5)ccc4n3)cc2o1] |
| 605 | 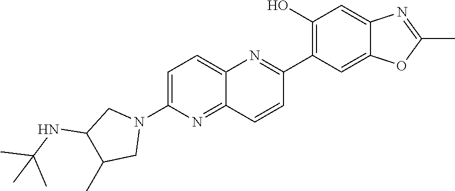 [SMILES: Cc1nc2cc(O)c(-c3ccc4nc(N5CC(C)C(NC(C)(C)C)C5)ccc4n3)cc2o1] |
| 606 | 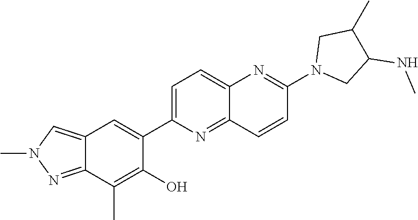 |
| 607 | 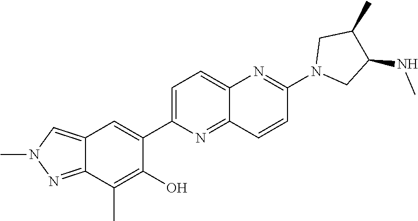 |
| 608 | 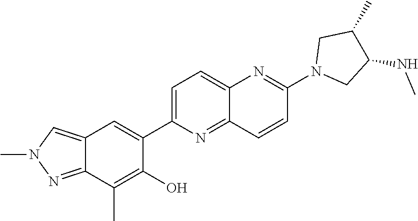 |
| 609 | 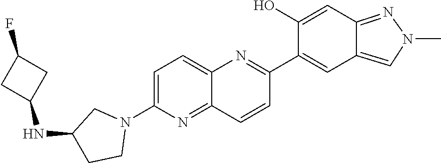 [SMILES: Cn1cc2cc(-c3ccc4nc(N5CC[C@@H](N[C@H]6C[C@@H](F)C6)C5)ccc4n3)c(O)cc2n1] |
| 610 | 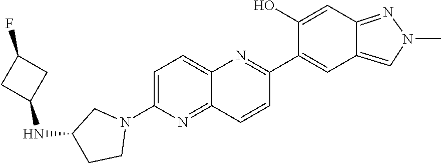 [SMILES: Cn1cc2cc(-c3ccc4nc(N5CC[C@H](N[C@H]6C[C@@H](F)C6)C5)ccc4n3)c(O)cc2n1] |
| 611 | 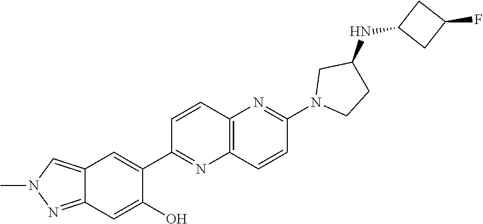 [SMILES: Cn1cc2cc(-c3ccc4nc(N5CC[C@H](N[C@H]6C[C@H](F)C6)C5)ccc4n3)c(O)cc2n1] |
| 612 | 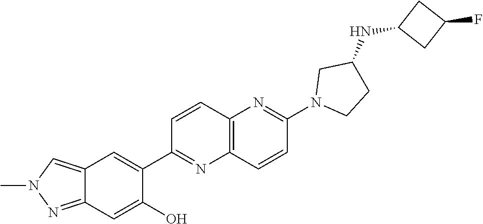 [SMILES: Cn1cc2cc(-c3ccc4nc(N5CC[C@@H](N[C@H]6C[C@H](F)C6)C5)ccc4n3)c(O)cc2n1] |
| 613 | 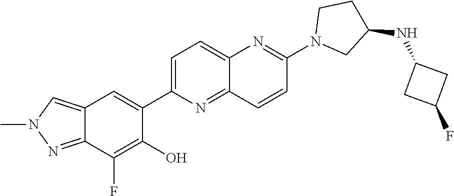 [SMILES: Cn1cc2cc(-c3ccc4nc(N5CC[C@@H](N[C@H]6C[C@H](F)C6)C5)ccc4n3)c(O)c(F)c2n1] |
| 614 | 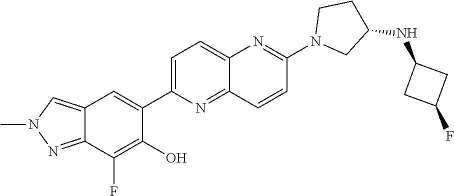 [SMILES: Cn1cc2cc(-c3ccc4nc(N5CC[C@H](N[C@H]6C[C@@H](F)C6)C5)ccc4n3)c(O)c(F)c2n1] |
| 615 | 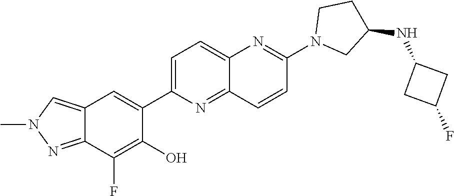 [SMILES: Cn1cc2cc(-c3ccc4nc(N5CC[C@@H](N[C@H]6C[C@@H](F)C6)C5)ccc4n3)c(O)c(F)c2n1] |
| 616 | 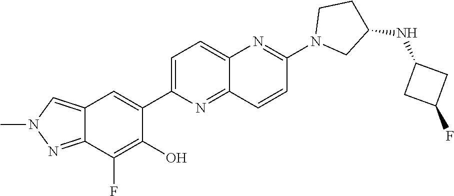 [SMILES: Cn1cc2cc(-c3ccc4nc(N5CC[C@H](N[C@H]6C[C@H](F)C6)C5)ccc4n3)c(O)c(F)c2n1] |
| 617 | 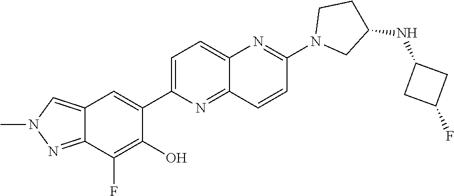 [SMILES: Cn1cc2cc(-c3ccc4nc(N5CC[C@H](N[C@H]6C[C@@H](F)C6)C5)ccc4n3)c(O)c(F)c2n1] |
| 618 | 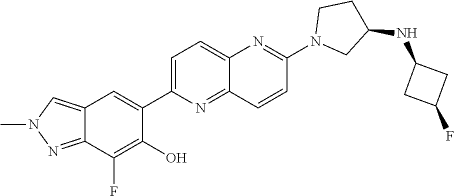 [SMILES: Cn1cc2cc(-c3ccc4nc(N5CC[C@@H](N[C@H]6C[C@@H](F)C6)C5)ccc4n3)c(O)c(F)c2n1] |
| 619 | 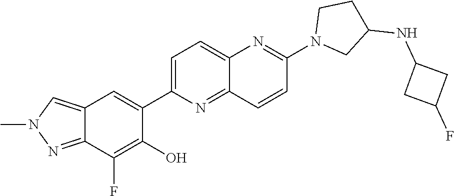 [SMILES: Cn1cc2cc(-c3ccc4nc(N5CCC(NC6CC(F)C6)C5)ccc4n3)c(O)c(F)c2n1] |
| 620 | 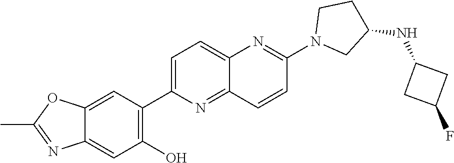 [SMILES: Cc1nc2cc(O)c(-c3ccc4nc(N5CC[C@H](N[C@H]6C[C@H](F)C6)C5)ccc4n3)cc2o1] |
| 621 | 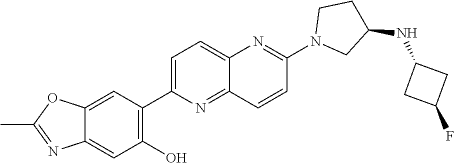 [SMILES: Cc1nc2cc(O)c(-c3ccc4nc(N5CC[C@@H](N[C@H]6C[C@H](F)C6)C5)ccc4n3)cc2o1] |
| 622 | 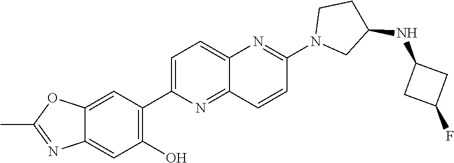 [SMILES: Cc1nc2cc(O)c(-c3ccc4nc(N5CC[C@@H](N[C@H]6C[C@@H](F)C6)C5)ccc4n3)cc2o1] |
| 623 | 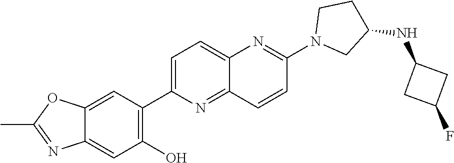 [SMILES: Cc1nc2cc(O)c(-c3ccc4nc(N5CC[C@H](N[C@H]6C[C@@H](F)C6)C5)ccc4n3)cc2o1] |
| 624 | 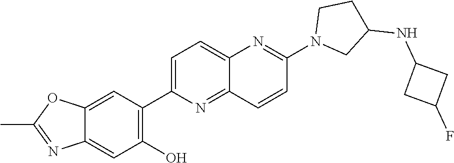 [SMILES: Cc1nc2cc(O)c(-c3ccc4nc(N5CCC(NC6CC(F)C6)C5)ccc4n3)cc2o1] |
| 625 | 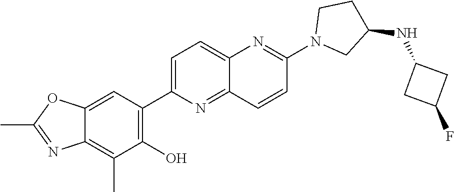 [SMILES: Cc1nc2c(C)c(O)c(-c3ccc4nc(N5CC[C@@H](N[C@H]6C[C@H](F)C6)C5)ccc4n3)cc2o1] |
| 626 | 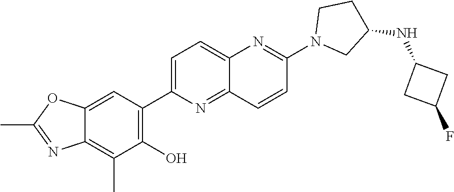 [SMILES: Cc1nc2c(C)c(O)c(-c3ccc4nc(N5CC[C@H](N[C@H]6C[C@H](F)C6)C5)ccc4n3)cc2o1] |
| 627 | 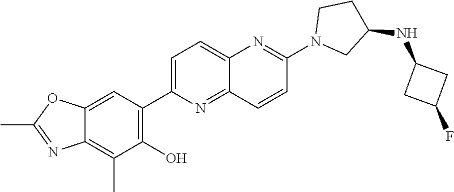 [SMILES: Cc1nc2c(C)c(O)c(-c3ccc4nc(N5CC[C@@H](N[C@H]6C[C@@H](F)C6)C5)ccc4n3)cc2o1] |
| 628 | 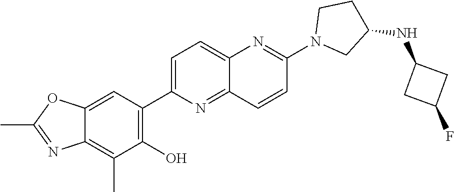 [SMILES: Cc1nc2c(C)c(O)c(-c3ccc4nc(N5CC[C@H](N[C@H]6C[C@@H](F)C6)C5)ccc4n3)cc2o1] |
| 629 | 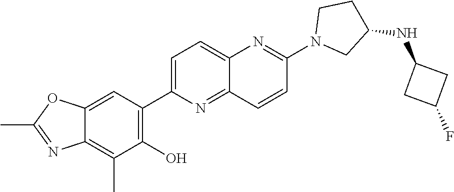 [SMILES: Cc1nc2c(C)c(O)c(-c3ccc4nc(N5CC[C@H](N[C@H]6C[C@H](F)C6)C5)ccc4n3)cc2o1] |
| 630 | 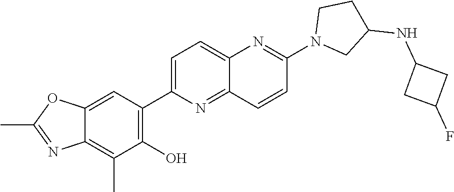 [SMILES: Cc1nc2c(C)c(O)c(-c3ccc4nc(N5CCC(NC6CC(F)C6)C5)ccc4n3)cc2o1] |
| 631 | 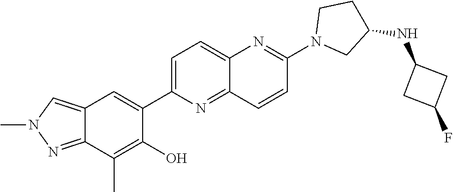 [SMILES: Cc1c(O)c(-c2ccc3nc(N4CC[C@H](N[C@H]5C[C@@H](F)C5)C4)ccc3n2)cc2cn(C)nc12] |
| 632 | 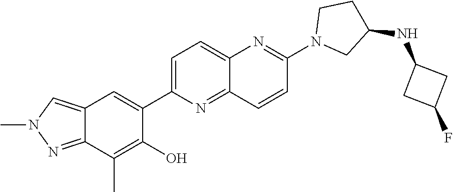 [SMILES: Cc1c(O)c(-c2ccc3nc(N4CC[C@@H](N[C@H]5C[C@@H](F)C5)C4)ccc3n2)cc2cn(C)nc12] |
| 633 | 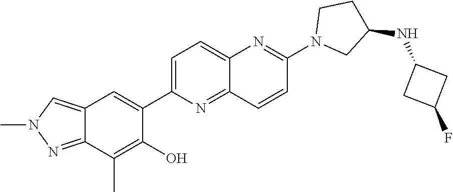 [SMILES: Cc1c(O)c(-c2ccc3nc(N4CC[C@@H](N[C@H]5C[C@H](F)C5)C4)ccc3n2)cc2cn(C)nc12] |
| 634 | 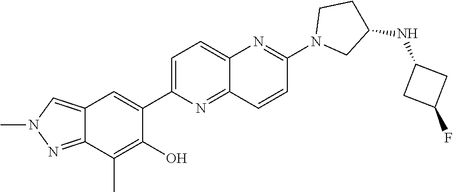 [SMILES: Cc1c(O)c(-c2ccc3nc(N4CC[C@H](N[C@H]5C[C@H](F)C5)C4)ccc3n2)cc2cn(C)nc12] |
| 635 | 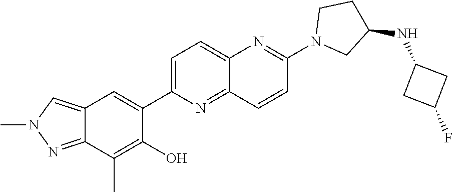 [SMILES: Cc1c(O)c(-c2ccc3nc(N4CC[C@@H](N[C@H]5C[C@@H](F)C5)C4)ccc3n2)cc2cn(C)nc12] |
| 636 | 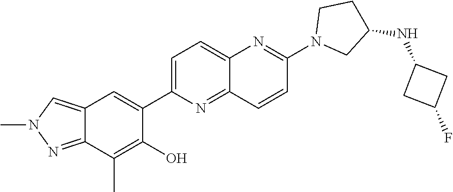 [SMILES: Cc1c(O)c(-c2ccc3nc(N4CC[C@H](N[C@H]5C[C@@H](F)C5)C4)ccc3n2)cc2cn(C)nc12] |
| 637 | 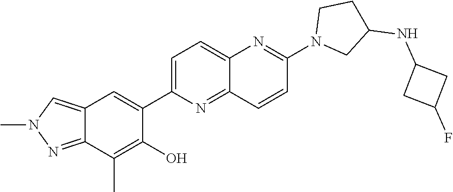 [SMILES: Cc1c(O)c(-c2ccc3nc(N4CCC(NC5CC(F)C5)C4)ccc3n2)cc2cn(C)nc12] |
| 638 | 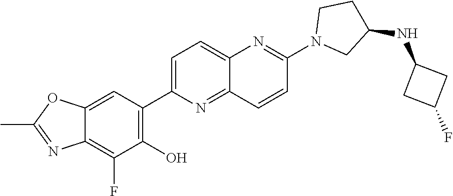 [SMILES: Cc1nc2c(F)c(O)c(-c3ccc4nc(N5CC[C@@H](N[C@H]6C[C@H](F)C6)C5)ccc4n3)cc2o1] |
| 639 | 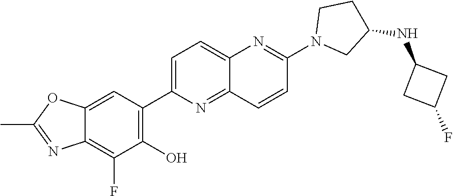 [SMILES: Cc1nc2c(F)c(O)c(-c3ccc4nc(N5CC[C@H](N[C@H]6C[C@H](F)C6)C5)ccc4n3)cc2o1] |
| 640 | 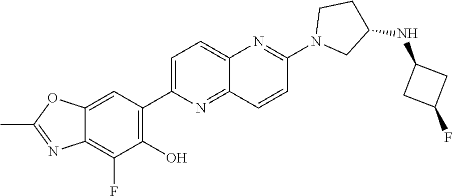 [SMILES: Cc1nc2c(F)c(O)c(-c3ccc4nc(N5CC[C@H](N[C@H]6C[C@@H](F)C6)C5)ccc4n3)cc2o1] |
| 641 | 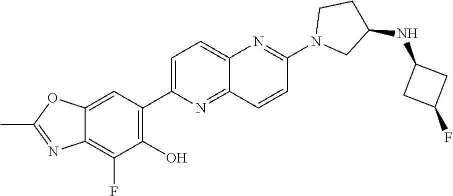 [SMILES: Cc1nc2c(F)c(O)c(-c3ccc4nc(N5CC[C@@H](N[C@H]6C[C@@H](F)C6)C5)ccc4n3)cc2o1] |
| 642 | 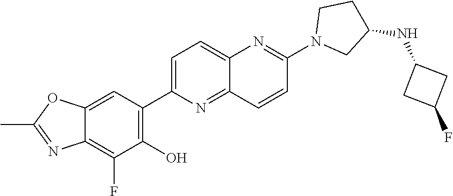 [SMILES: Cc1nc2c(F)c(O)c(-c3ccc4nc(N5CC[C@H](N[C@H]6C[C@H](F)C6)C5)ccc4n3)cc2o1] |
| 643 | 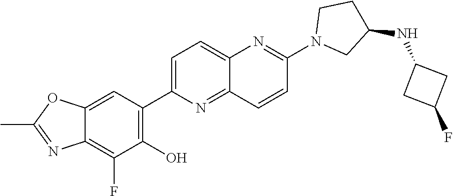 [SMILES: Cc1nc2c(F)c(O)c(-c3ccc4nc(N5CC[C@@H](N[C@H]6C[C@H](F)C6)C5)ccc4n3)cc2o1] |
| 644 | 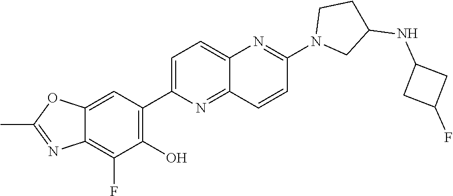 [SMILES: Cc1nc2c(F)c(O)c(-c3ccc4nc(N5CCC(NC6CC(F)C6)C5)ccc4n3)cc2o1] |
| 645 | 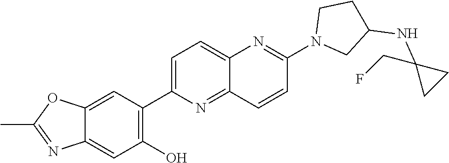 [SMILES: Cc1nc2cc(O)c(-c3ccc4nc(N5CCC(NC6(CF)CC6)C5)ccc4n3)cc2o1] |
| 646 | 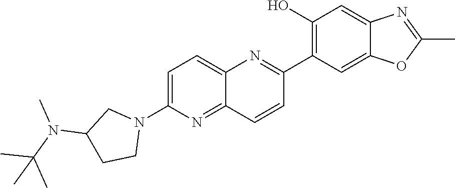 [SMILES: Cc1nc2cc(O)c(-c3ccc4nc(N5CCC(N(C)C(C)(C)C)C5)ccc4n3)cc2o1] |
| 647 | 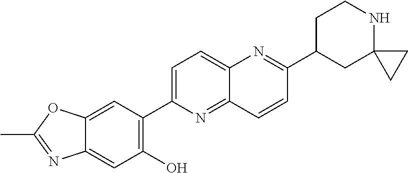 [SMILES: Cc1nc2cc(O)c(-c3ccc4nc(C5CCNC6(CC6)C5)ccc4n3)cc2o1] |
| 648 | 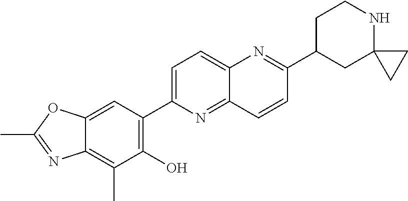 [SMILES: Cc1nc2c(C)c(O)c(-c3ccc4nc(C5CCNC6(CC6)C5)ccc4n3)cc2o1] |
| 649 | 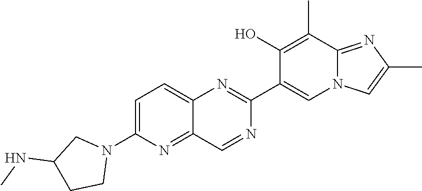 |
| 650 | 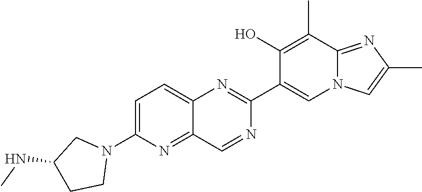 |
| 651 | 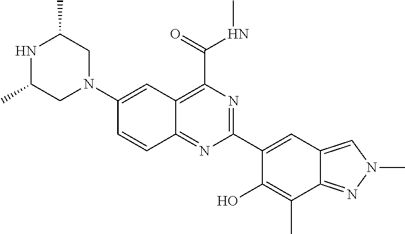 |
| 652 | 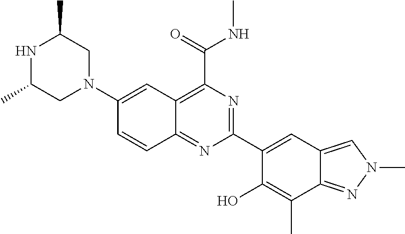 |
| 653 | 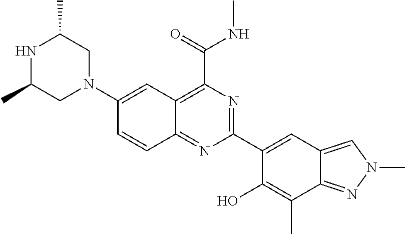 |
| 654 | 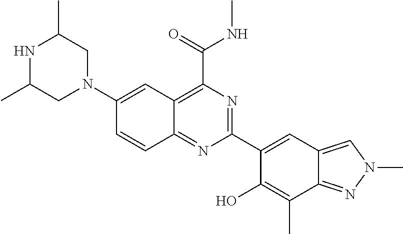 |
| 655 | 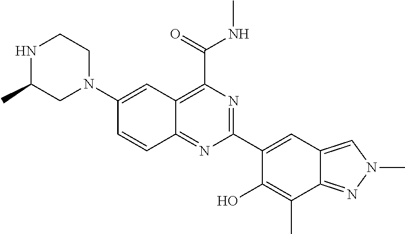 |
| 656 | 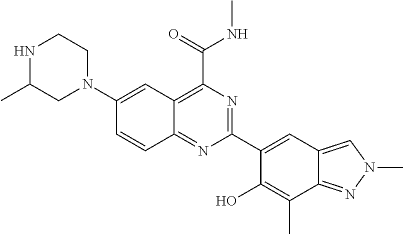 |
| 657 | 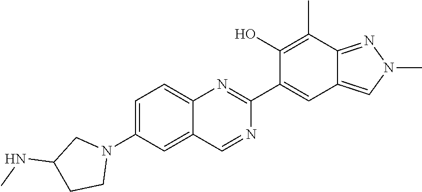 |
| 658 | 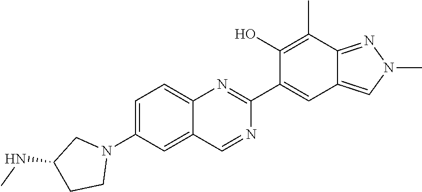 |
| 659 | 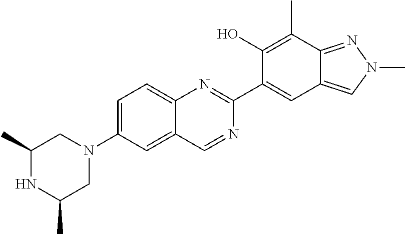 |
| 660 | 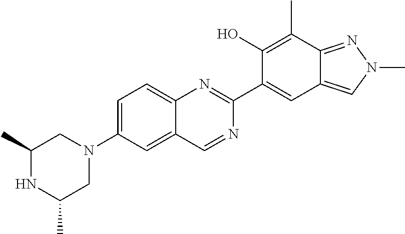 |
| 661 | 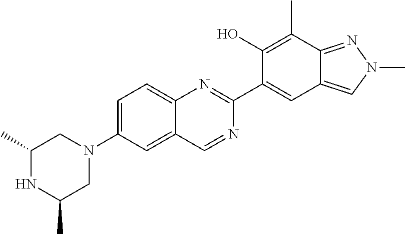 |
| 662 | 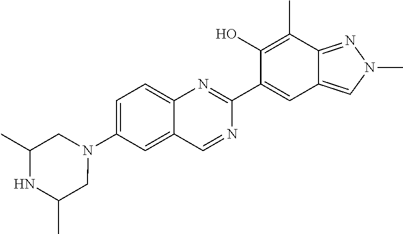 |
| 663 | 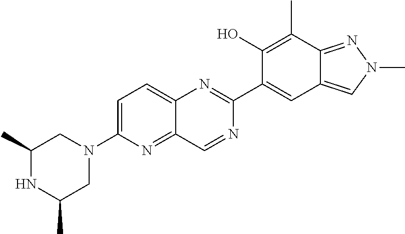 |
| 664 | 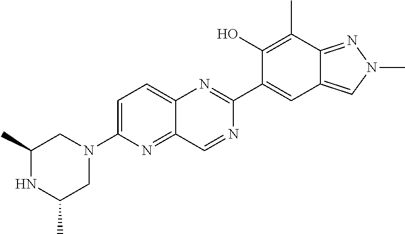 |
| 665 | 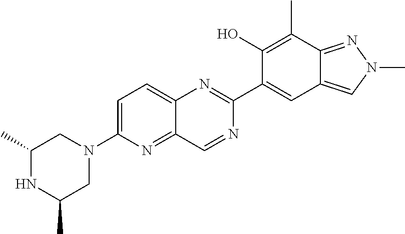 |
| 666 | 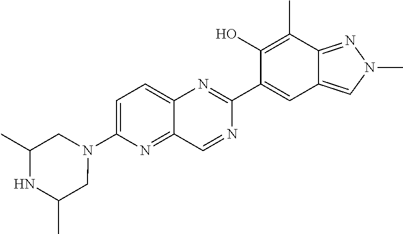 |
| 667 | 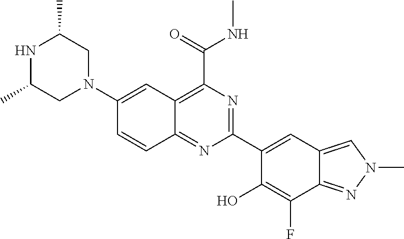 |
| 668 | 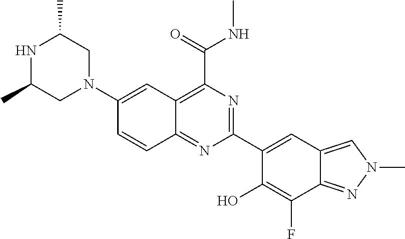 |
| 669 | 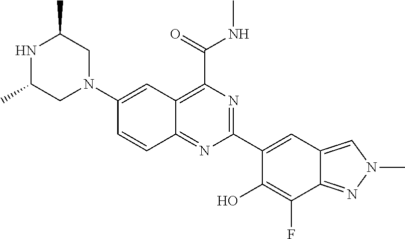 |
| 670 | 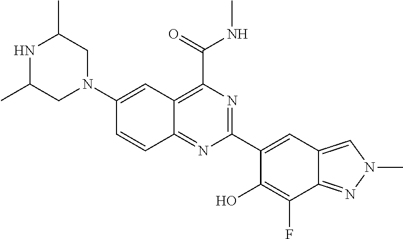 |
| 671 | 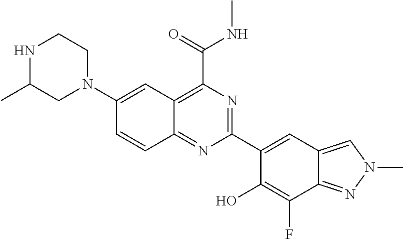 |
| 672 | 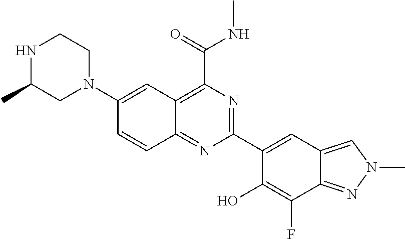 |
| 673 | 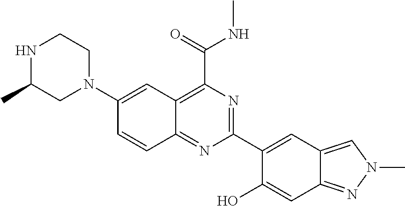 |
| 674 | 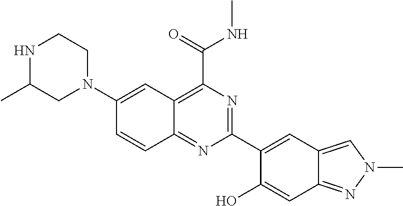 |
| 675 | 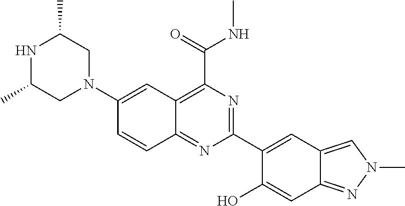 |
| 676 | 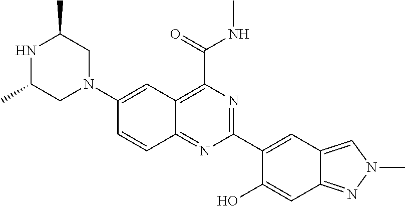 |
| 677 | 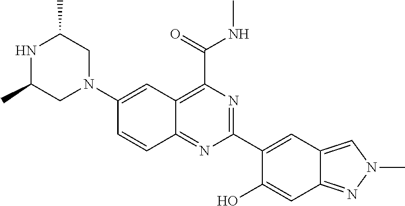 |
| 678 | 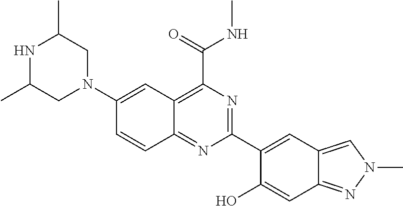 |
| 679 | 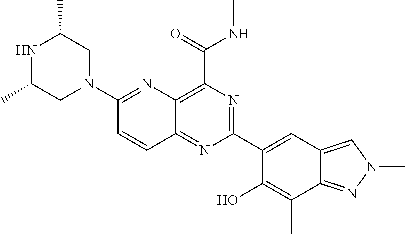 |
| 680 | 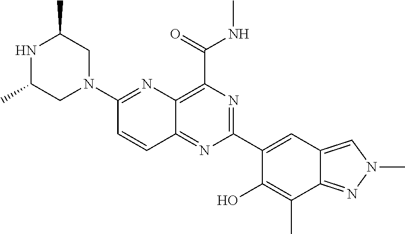 |
| 681 | 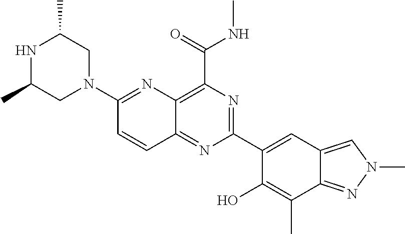 |
| 682 | 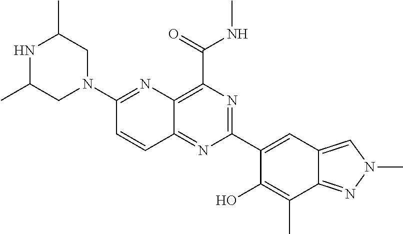 |
| 683 | 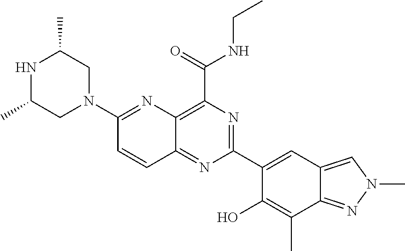 |
| 684 | 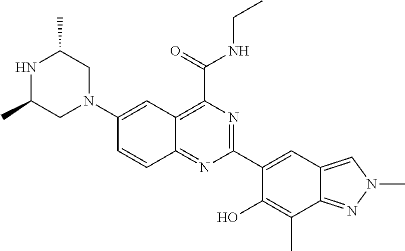 |
| 685 | 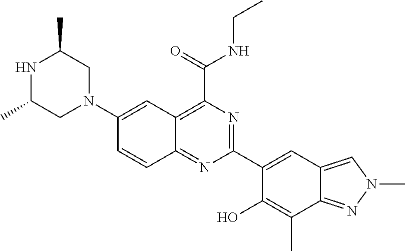 |
| 686 | 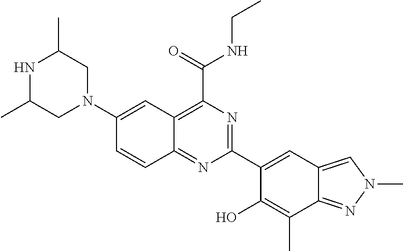 |
| 687 | 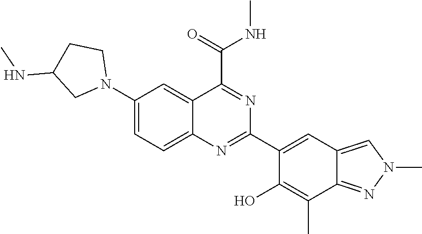 |
| 688 | 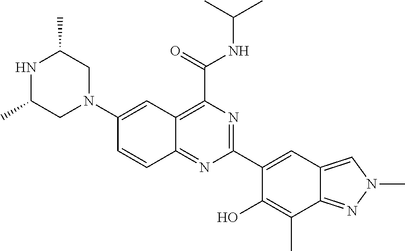 |
| 689 | 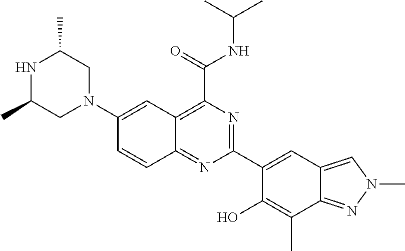 |
| 690 | 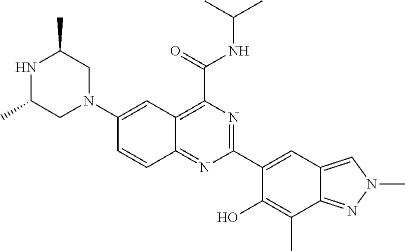 |
| 691 | 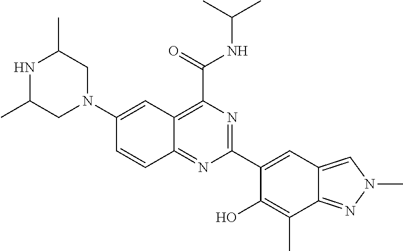 |
| 692 | 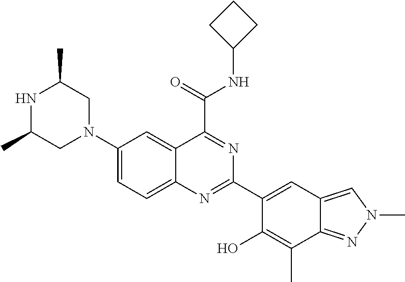 |
| 693 | 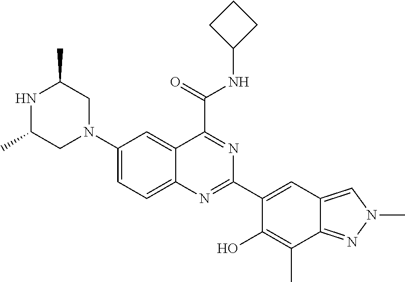 |
| 694 | 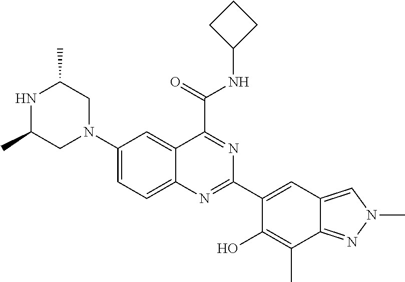 |
| 695 | 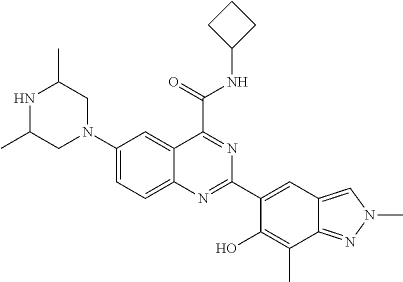 |
| 696 | 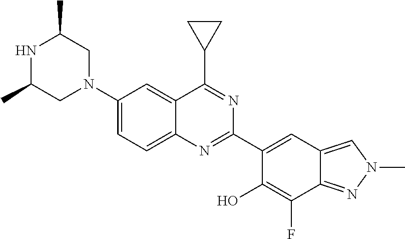 |
| 697 | 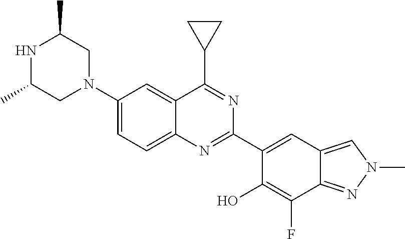 |
| 698 | 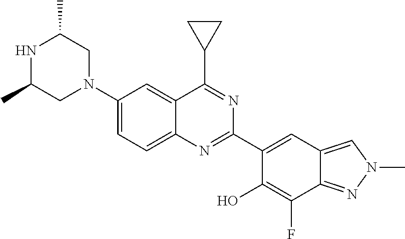 |
| 699 | 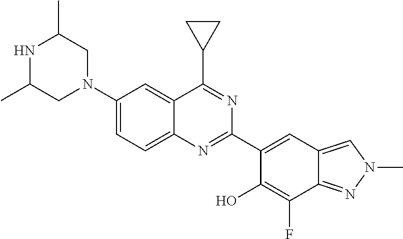 |
| 700 | 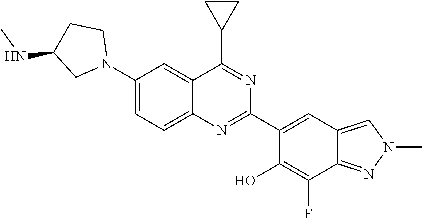 |
| 701 | 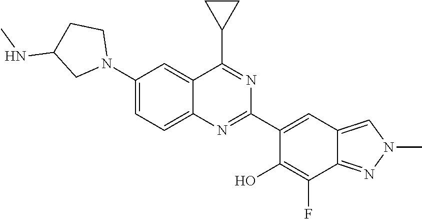 |
| 702 | 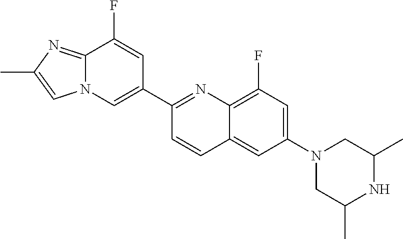 |
| 703 | 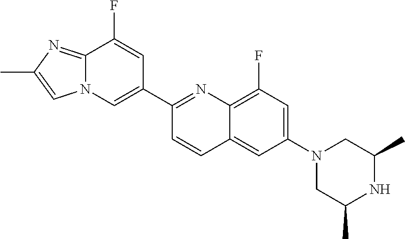 |
| 704 | 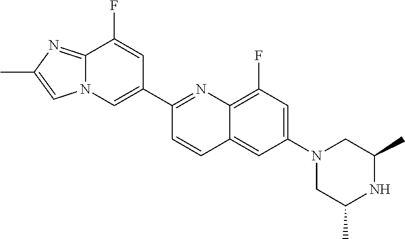 |
| 705 | 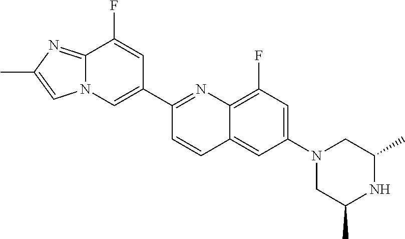 |
| 706 | 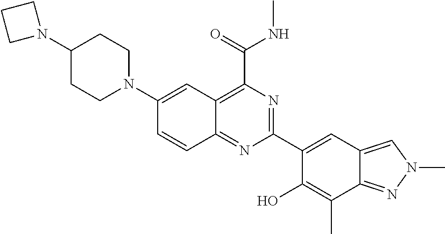 [SMILES: CNC(=O)c1nc(-c2cc3cn(C)nc3c(C)c2O)nc2ccc(N3CCC(N4CCC4)CC3)cc12] |
| 707 | 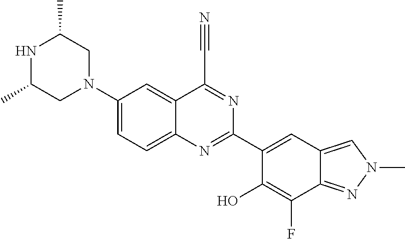 |
| 708 | 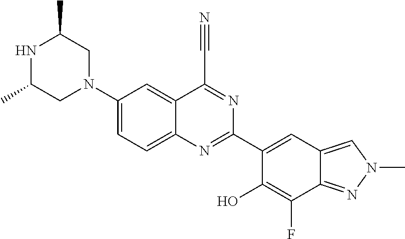 |
| 709 | 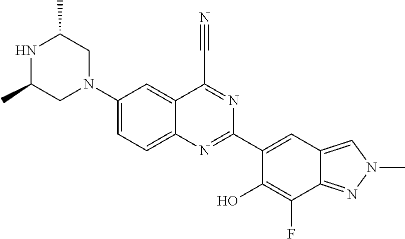 |
| 710 | 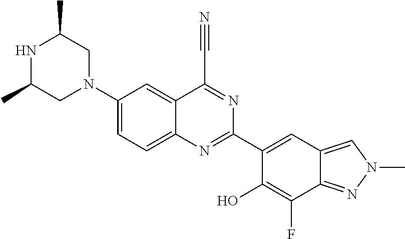 |
| 711 | 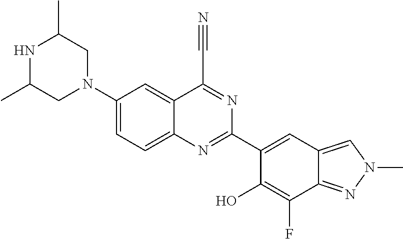 |
| 712 | 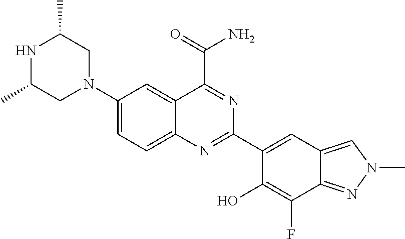 |
| 713 | 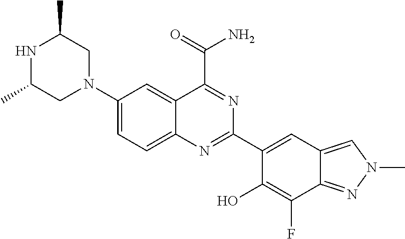 |
| 714 | 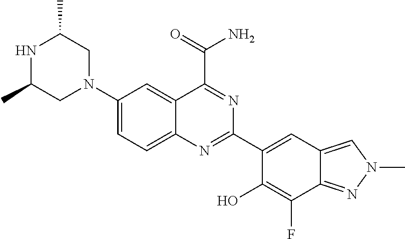 |
| 715 | 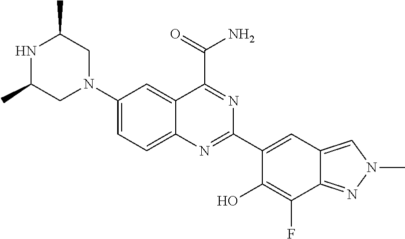 |
| 716 | 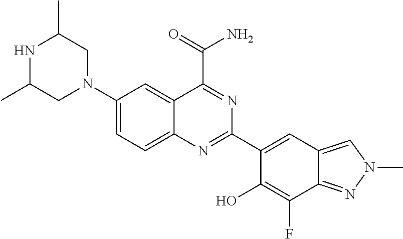 |
| 717 | 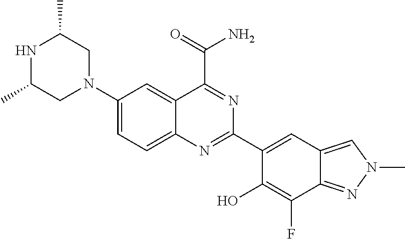 |
| 718 | 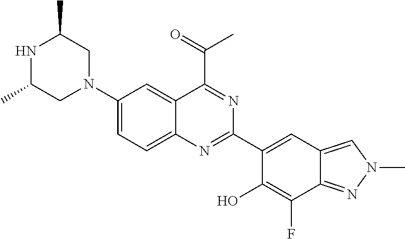 |
| 719 | 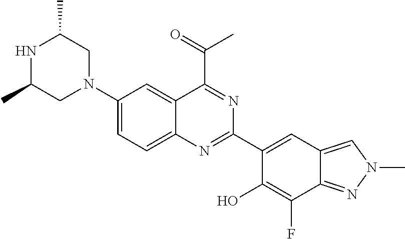 |
| 720 | 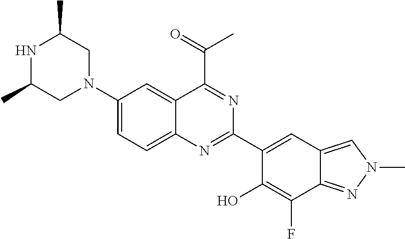 |
| 721 | 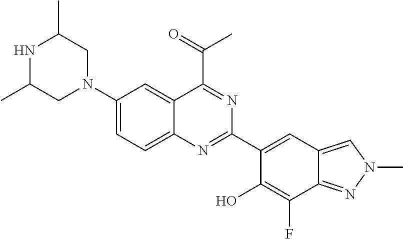 |
| 722 | 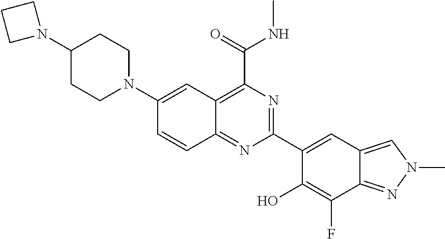 [SMILES: CNC(=O)c1nc(-c2cc3cn(C)nc3c(F)c2O)nc2ccc(N3CCC(N4CCC4)CC3)cc12] |
| 723 | 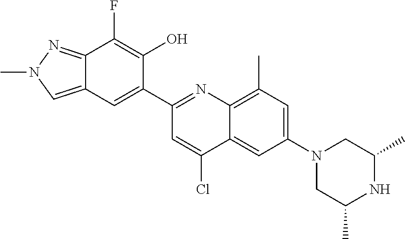 |
| 724 | 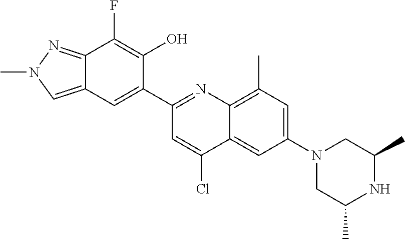 |
| 725 | 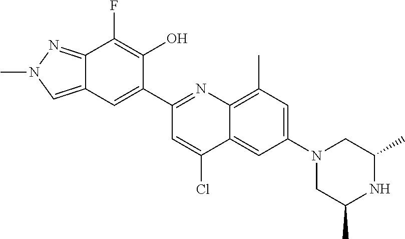 |
| 726 | 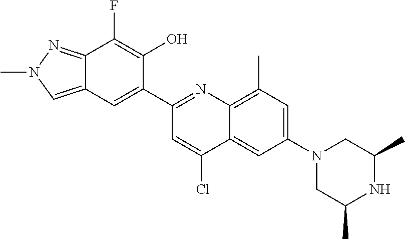 |
| 727 | 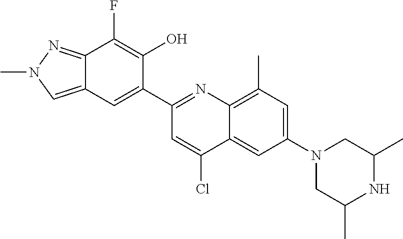 |
| 728 | 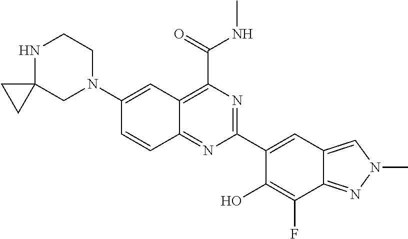 [SMILES: CNC(=O)c1nc(-c2cc3cn(C)nc3c(F)c2O)nc2ccc(N3CCNC4(CC4)C3)cc12] |
| 729 | 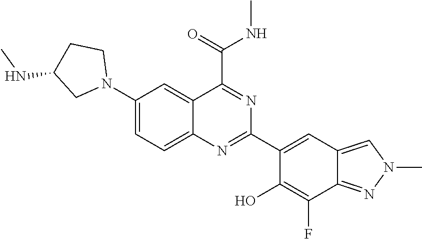 |
| 730 | 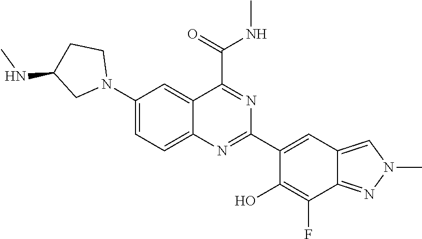 |
| 731 | 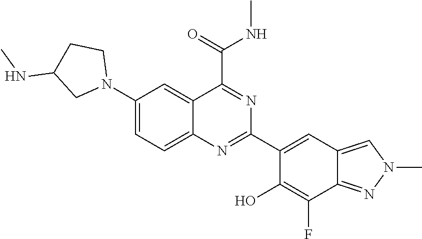 |
| 732 | 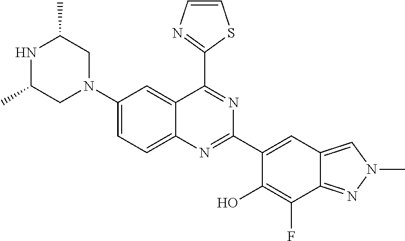 |
| 733 | 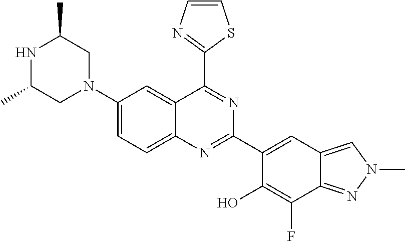 |
| 734 | 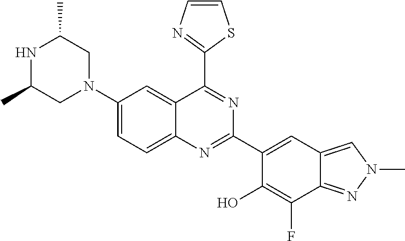 |
| 735 | 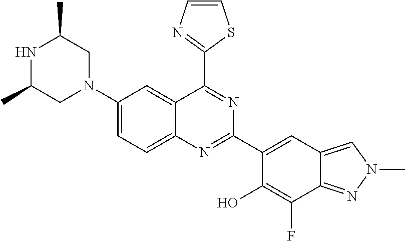 |
| 736 | 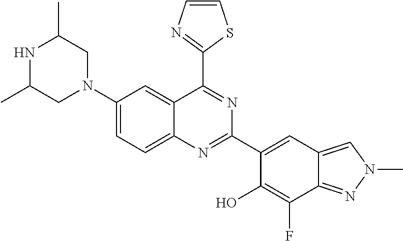 |
| 737 | 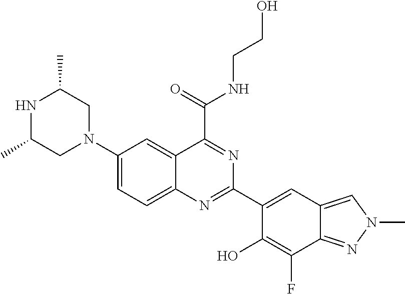 |
| 738 | 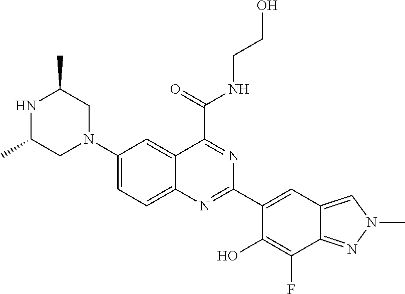 |
| 739 | 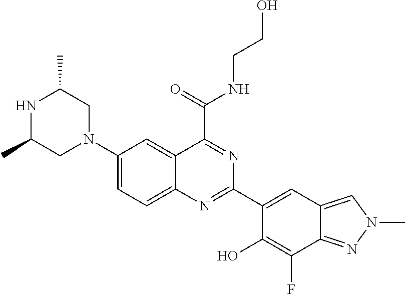 |
| 740 | 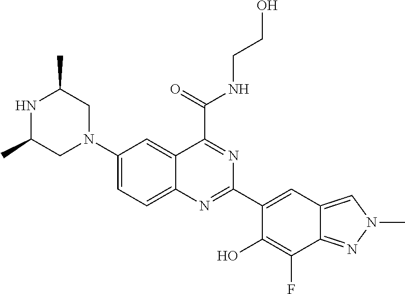 |
| 741 | 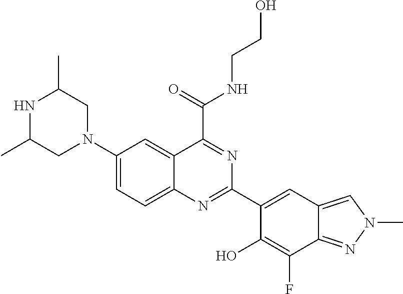 |
| 742 | 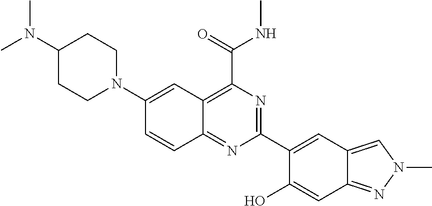 |
| 743 | 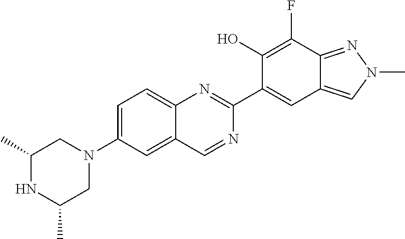 |
| 744 | 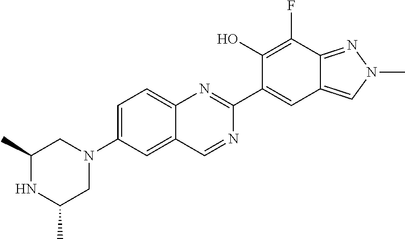 |
| 745 | 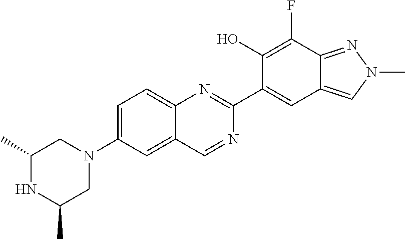 |
| 746 | 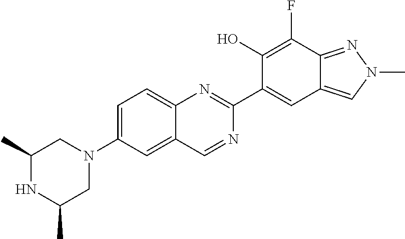 |
| 747 | 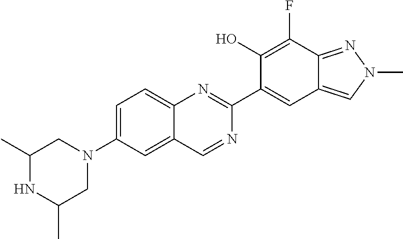 |
| 748 | 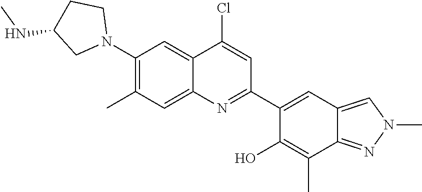 |
| 749 | 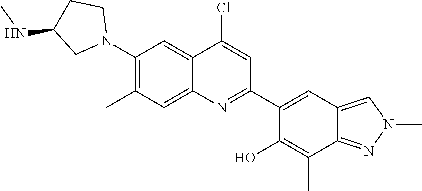 |
| 750 | 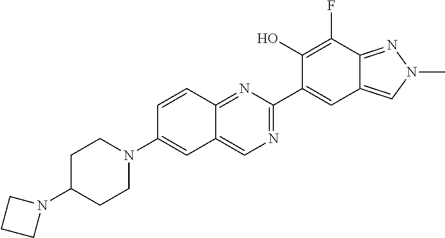 [SMILES: Cn1cc2cc(-c3ncc4cc(N5CCC(N6CCC6)CC5)ccc4n3)c(O)c(F)c2n1] |
| 751 | 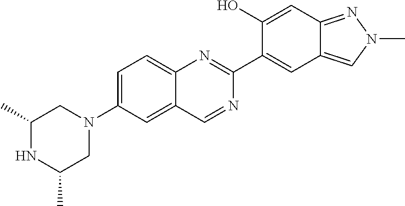 |
| 752 | 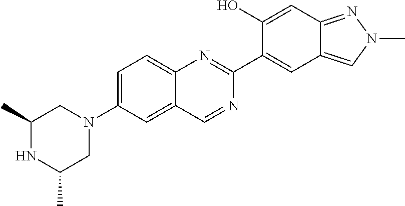 |
| 753 | 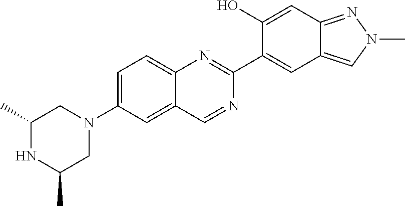 |
| 754 | 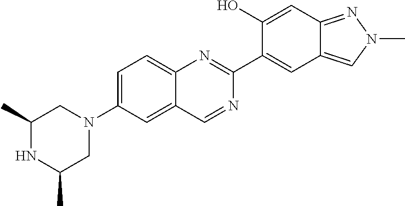 |
| 755 | 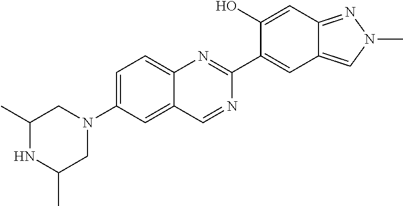 |
| 756 | 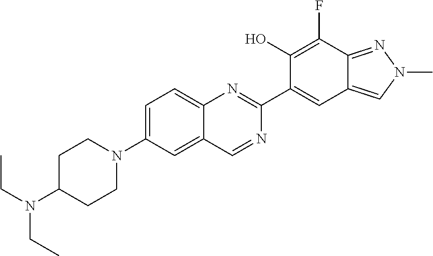 [SMILES: CCN(CC)C1CCN(c2ccc3nc(-c4cc5cn(C)nc5c(F)c4O)ncc3c2)CC1] |
| 757 | 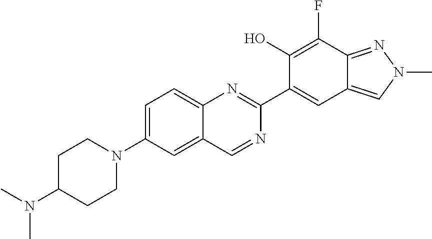 |
| 758 | 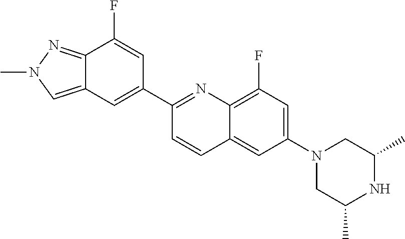 |
| 759 | 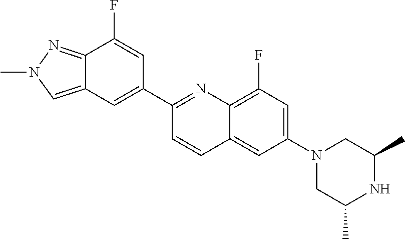 |
| 760 | 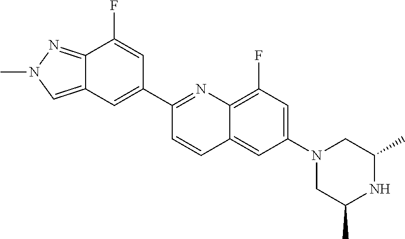 |
| 761 | 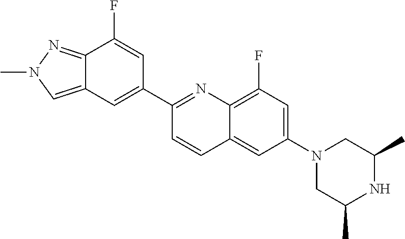 |
| 762 | 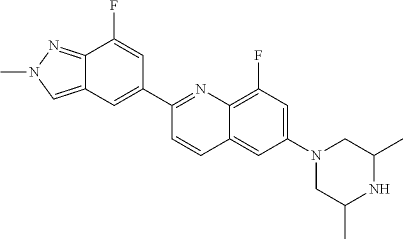 |
| 763 | 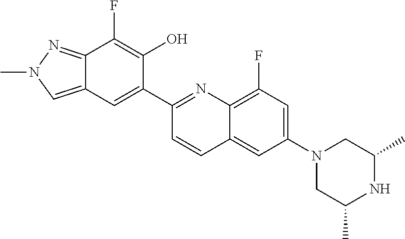 |
| 764 | 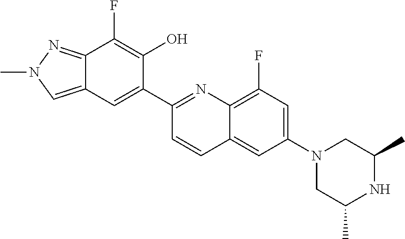 |
| 765 | 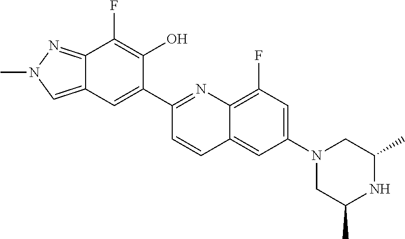 |
| 766 | 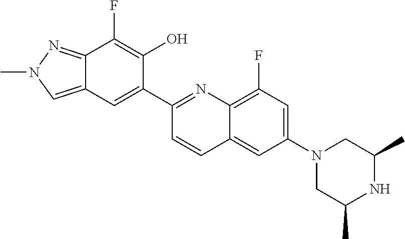 |
| 767 | 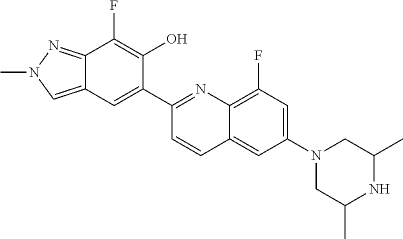 |
| 768 | 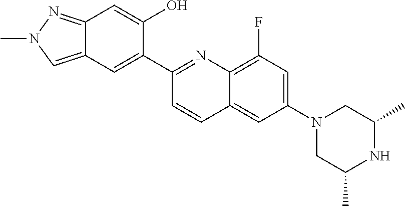 |
| 769 | 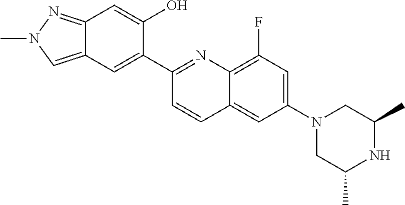 |
| 770 | 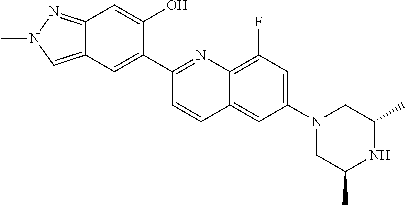 |
| 771 | 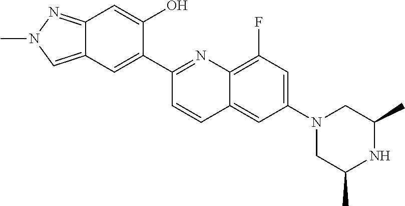 |
| 772 | 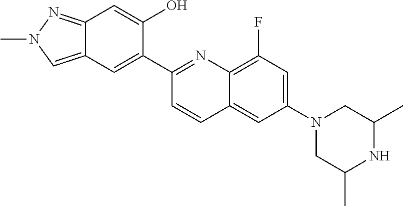 |
| 773 | 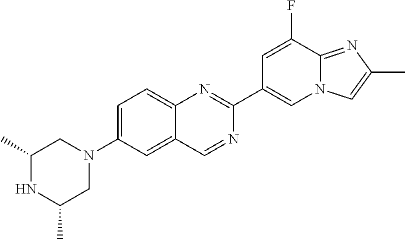 |
| 774 | 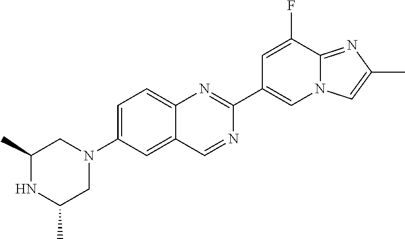 |
| 775 | 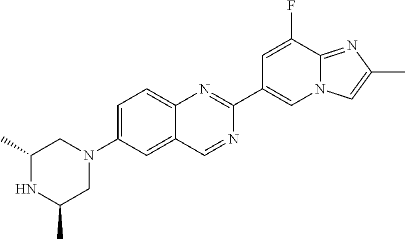 |
| 776 | 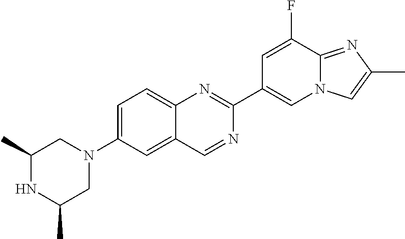 |
| 777 | 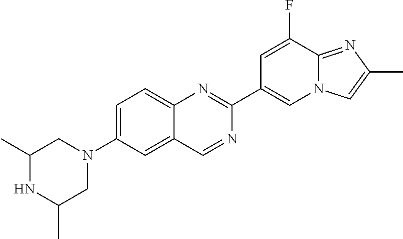 |
| 778 | 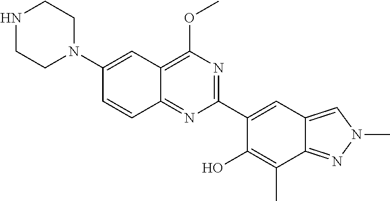 [SMILES: COc1nc(-c2cc3cn(C)nc3c(C)c2O)nc2ccc(N3CCNCC3)cc12] |
| 779 | 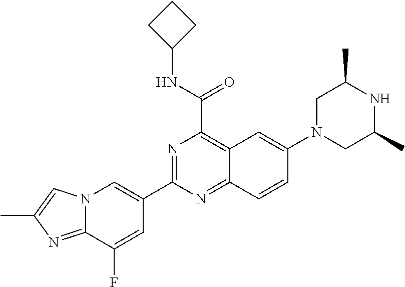 |
| 780 | 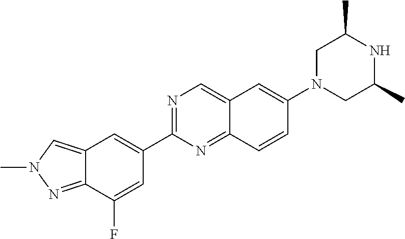 |
| 781 | 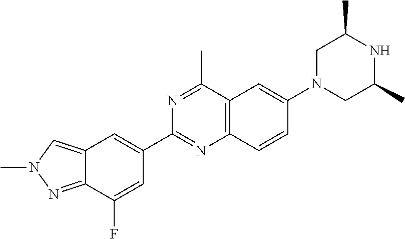 |
| 782 | 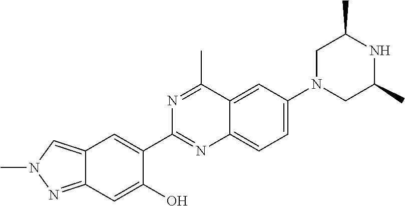 |
| 783 | 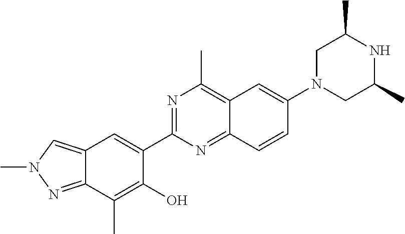 |
| 784 | 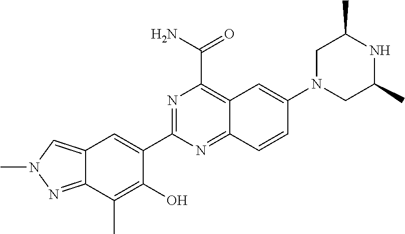 |
| 785 | 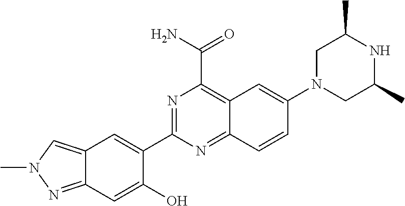 |
| 786 | 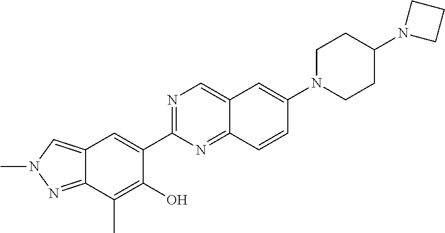 [SMILES: Cc1c(O)c(-c2ncc3cc(N4CCC(N5CCC5)CC4)ccc3n2)cc2cn(C)nc12] |
| 787 | 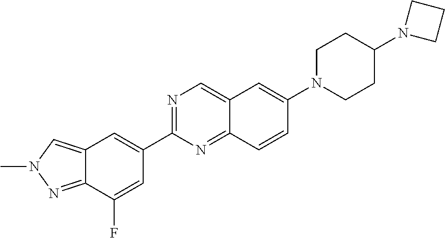 [SMILES: Cn1cc2cc(-c3ncc4cc(N5CCC(N6CCC6)CC5)ccc4n3)cc(F)c2n1] |
| 788 | 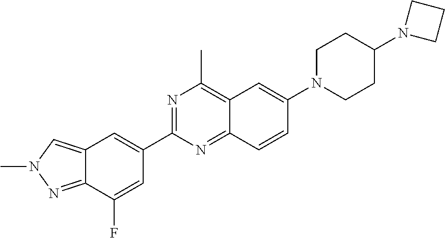 [SMILES: Cc1nc(-c2cc(F)c3nn(C)cc3c2)nc2ccc(N3CCC(N4CCC4)CC3)cc12] |
| 789 | 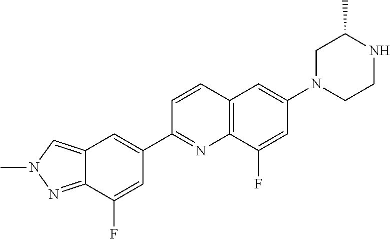 [SMILES: C[C@H]1CN(c2cc(F)c3nc(-c4cc(F)c5nn(C)cc5c4)ccc3c2)CCN1] |
| 790 | 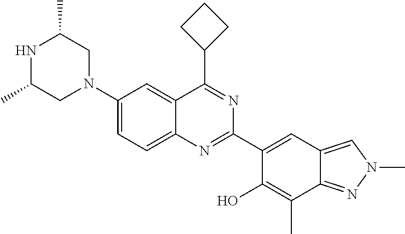 |
| 791 | 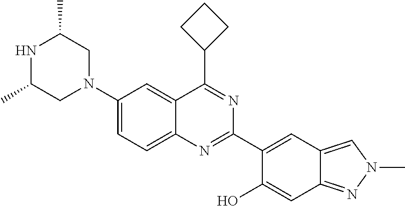 |
| 792 | 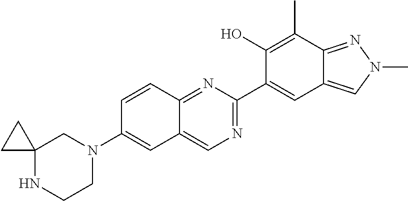 [SMILES: Cc1c(O)c(-c2ncc3cc(N4CCNC5(CC5)C4)ccc3n2)cc2cn(C)nc12] |
| 793 | 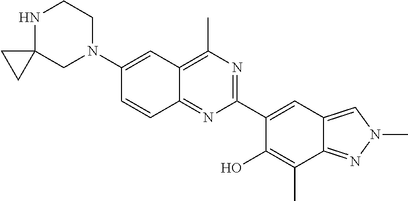 [SMILES: Cc1nc(-c2cc3cn(C)nc3c(C)c2O)nc2ccc(N3CCNC4(CC4)C3)cc12] |
| 794 | 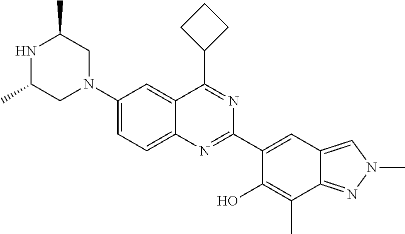 |
| 795 | 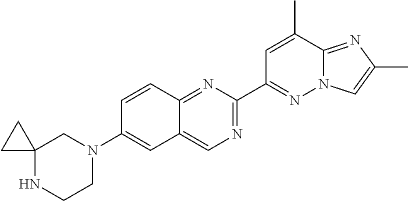 [SMILES: Cc1cn2nc(-c3ncc4cc(N5CCNC6(CC6)C5)ccc4n3)cc(C)c2n1] |
| 796 | 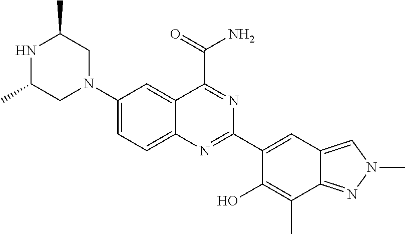 |
| 797 | 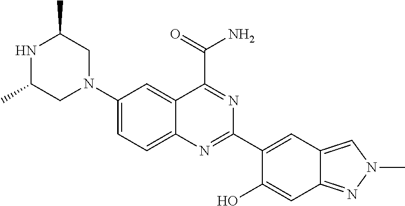 |
| 798 | 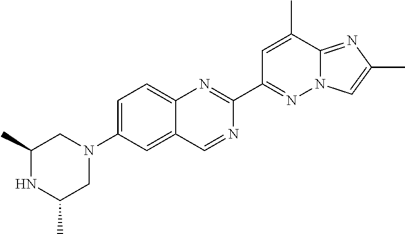 |
| 799 | 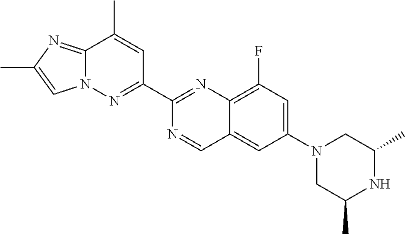 |
| 800 | 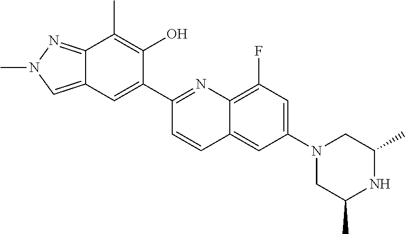 |
| 801 | 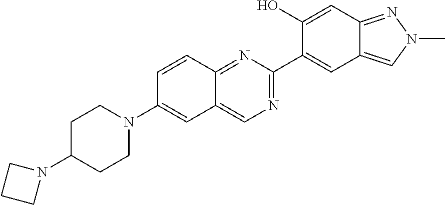 [SMILES: Cn1cc2cc(-c3ncc4cc(N5CCC(N6CCC6)CC5)ccc4n3)c(O)cc2n1] |
| 802 | 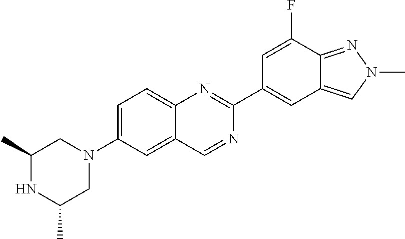 |
| 803 | 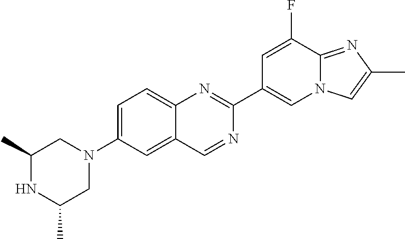 |
| 804 | 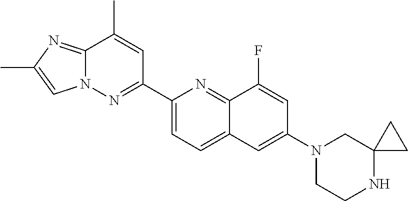 [SMILES: Cc1cn2nc(-c3ccc4cc(N5CCNC6(CC6)C5)cc(F)c4n3)cc(C)c2n1] |
| 805 | 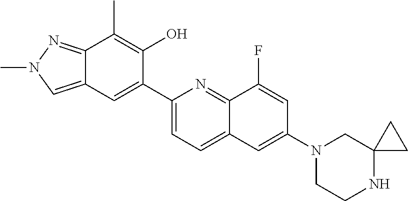 [SMILES: Cc1c(O)c(-c2ccc3cc(N4CCNC5(CC5)C4)cc(F)c3n2)cc2cn(C)nc12] |
| 806 | 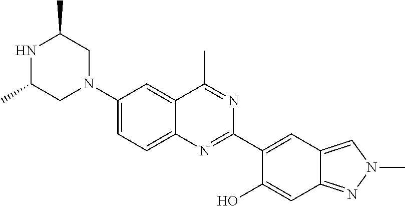 |
| 807 | 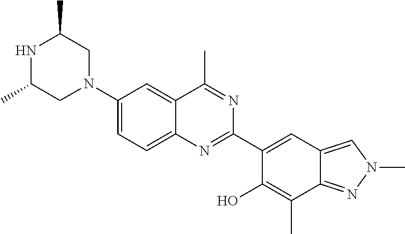 |
| 808 | 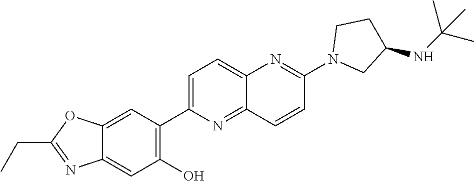 [SMILES: CCc1nc2cc(O)c(-c3ccc4nc(N5CC[C@@H](NC(C)(C)C)C5)ccc4n3)cc2o1] |
| 809 | 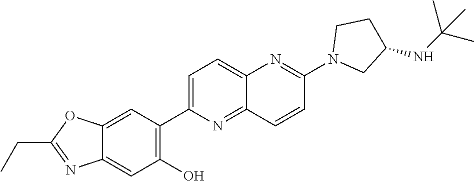 [SMILES: CCc1nc2cc(O)c(-c3ccc4nc(N5CC[C@H](NC(C)(C)C)C5)ccc4n3)cc2o1] |
| 810 | 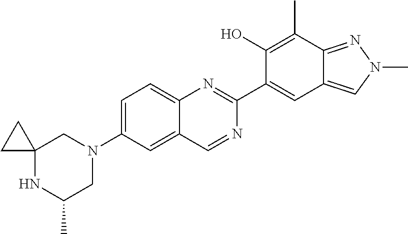 [SMILES: Cc1c(O)c(-c2ncc3cc(N4C[C@H](C)NC5(CC5)C4)ccc3n2)cc2cn(C)nc12] |
| 811 | 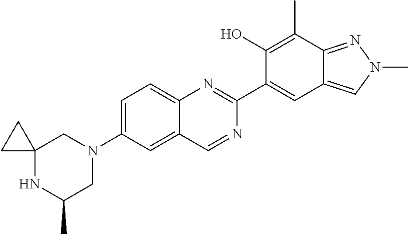 [SMILES: Cc1c(O)c(-c2ncc3cc(N4C[C@@H](C)NC5(CC5)C4)ccc3n2)cc2cn(C)nc12] |
| 812 | 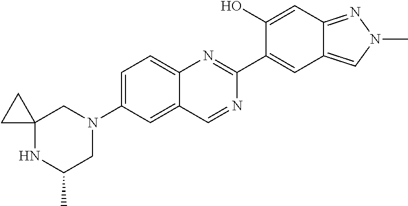 [SMILES: C[C@H]1CN(c2ccc3nc(-c4cc5cn(C)nc5cc4O)ncc3c2)CC2(CC2)N1] |
| 813 | 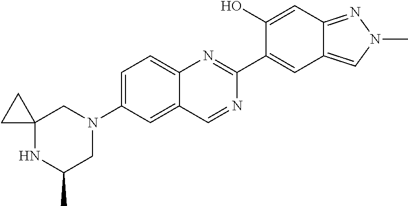 [SMILES: C[C@@H]1CN(c2ccc3nc(-c4cc5cn(C)nc5cc4O)ncc3c2)CC2(CC2)N1] |
| 814 | 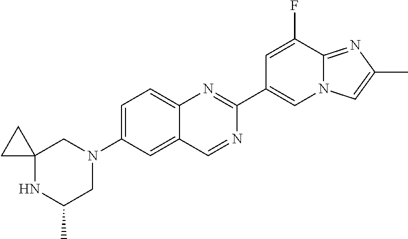 [SMILES: Cc1cn2cc(-c3ncc4cc(N5C[C@H](C)NC6(CC6)C5)ccc4n3)cc(F)c2n1] |
| 815 | 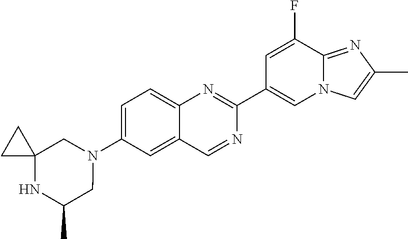 [SMILES: Cc1cn2cc(-c3ncc4cc(N5C[C@@H](C)NC6(CC6)C5)ccc4n3)cc(F)c2n1] |
| 816 | 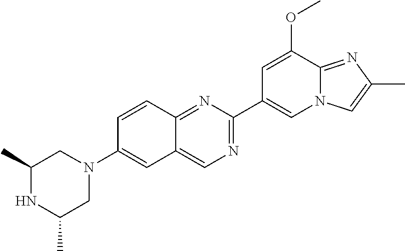 |
| 817 | 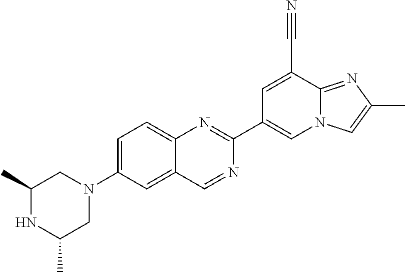 |
| 818 | 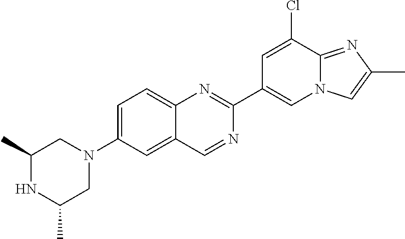 |
| 819 | 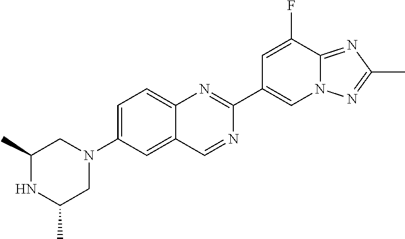 |
| 820 | 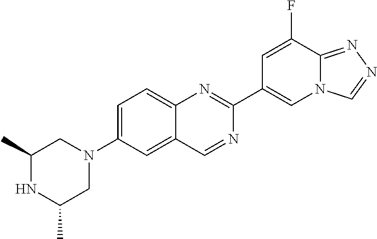 [SMILES: C[C@H]1CN(c2ccc3nc(-c4cc(F)c5nncn5c4)ncc3c2)C[C@H](C)N1] |
| 821 | 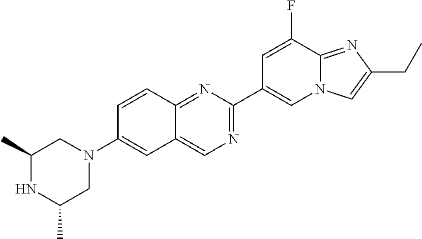 [SMILES: CCc1cn2cc(-c3ncc4cc(N5C[C@H](C)N[C@@H](C)C5)ccc4n3)cc(F)c2n1] |
| 822 | 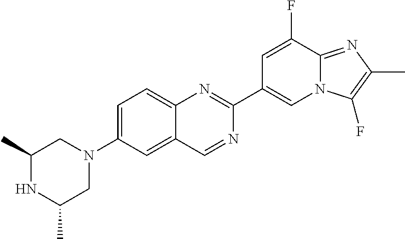 |
| 823 | 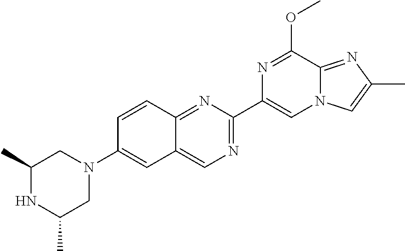 |
| 824 | 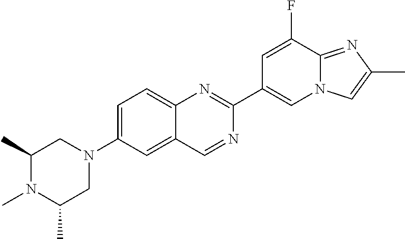 |
| 825 | 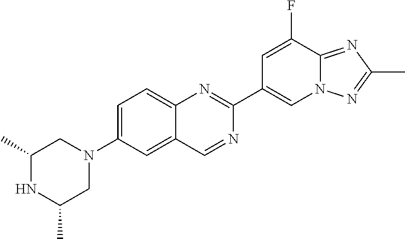 |
| 826 | 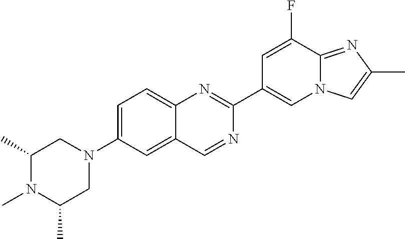 |
| 827 | 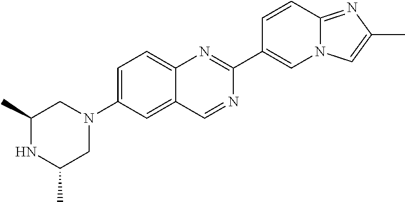 |
| 828 | 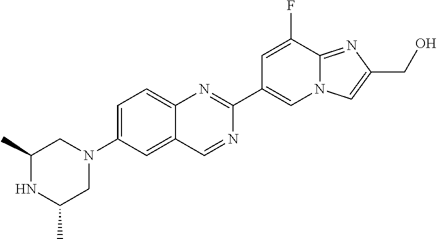 [SMILES: C[C@H]1CN(c2ccc3nc(-c4cc(F)c5nc(CO)cn5c4)ncc3c2)C[C@H](C)N1] |
| 829 | 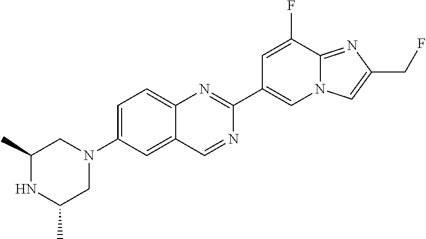 [SMILES: C[C@H]1CN(c2ccc3nc(-c4cc(F)c5nc(CF)cn5c4)ncc3c2)C[C@H](C)N1] |
| 830 | 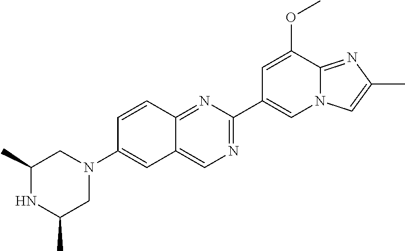 |
| 831 | 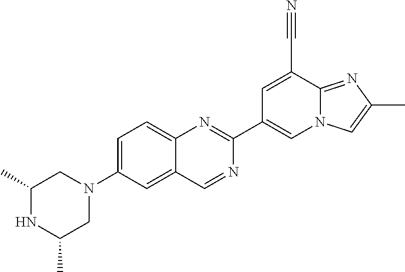 |
| 832 | 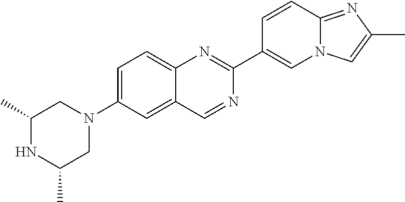 |
| 833 | 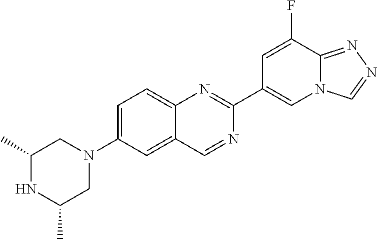 [SMILES: C[C@@H]1CN(c2ccc3nc(-c4cc(F)c5nncn5c4)ncc3c2)C[C@H](C)N1] |
| 834 | 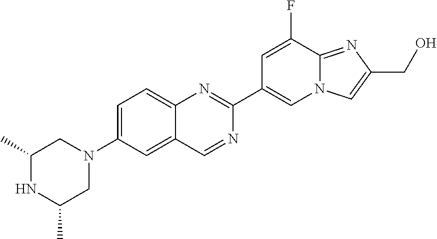 [SMILES: C[C@@H]1CN(c2ccc3nc(-c4cc(F)c5nc(CO)cn5c4)ncc3c2)C[C@H](C)N1] |
| 835 | 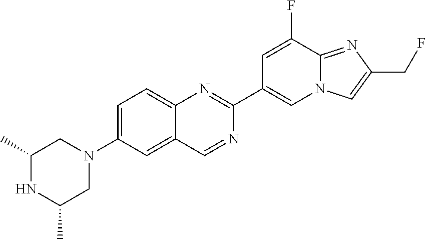 [SMILES: C[C@@H]1CN(c2ccc3nc(-c4cc(F)c5nc(CF)cn5c4)ncc3c2)C[C@H](C)N1] |
| 836 | 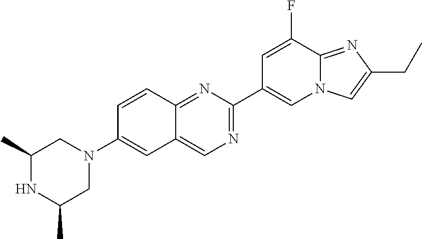 [SMILES: CCc1cn2cc(-c3ncc4cc(N5C[C@@H](C)N[C@@H](C)C5)ccc4n3)cc(F)c2n1] |
| 837 | 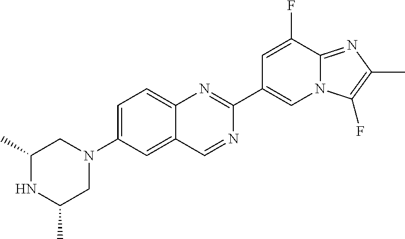 |
| 838 | 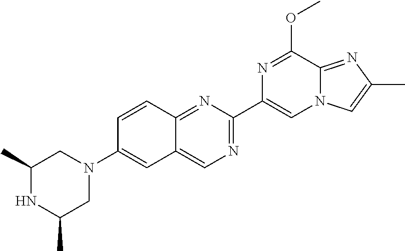 |
| 839 | 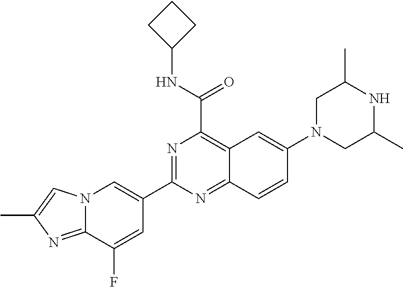 |
| 840 | 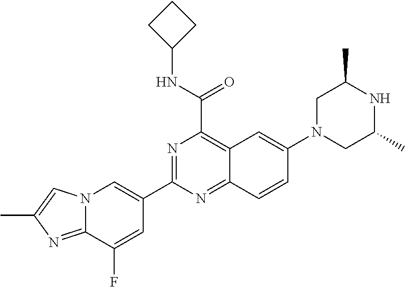 |
| 841 | 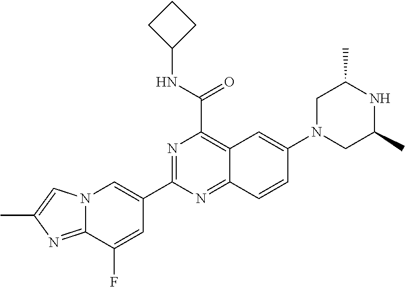 |
| 842 | 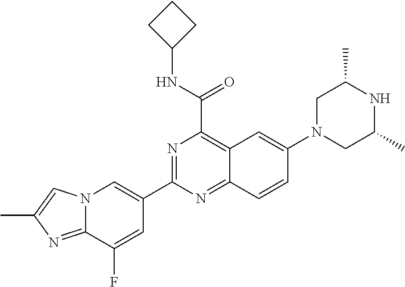 |
| 843 | 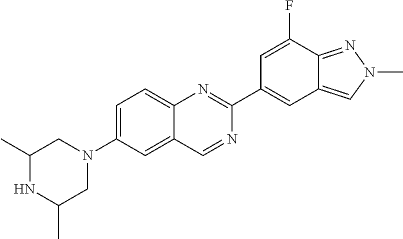 |
| 844 | 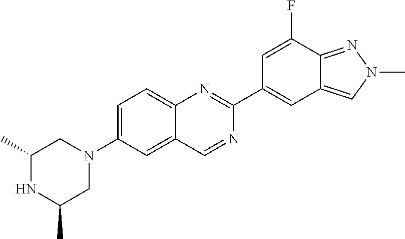 |
| 845 | 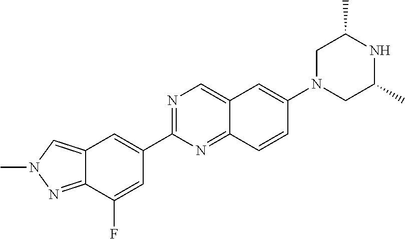 |
| 846 | 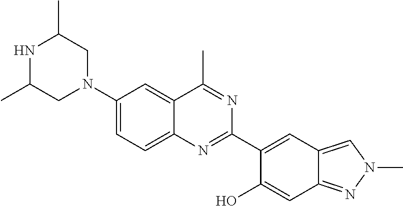 |
| 847 | 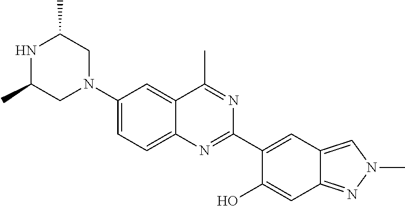 |
| 848 | 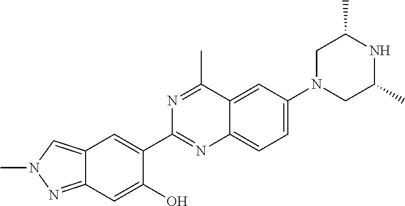 |
| 849 | 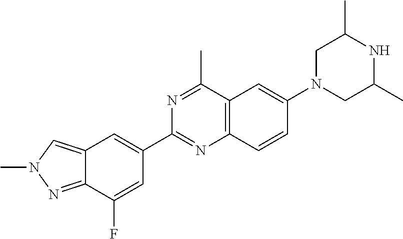 |
| 850 | 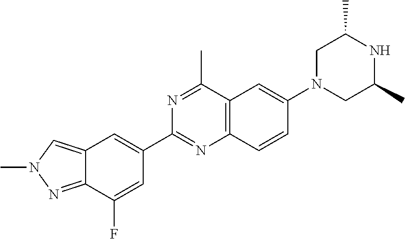 |
| 851 | 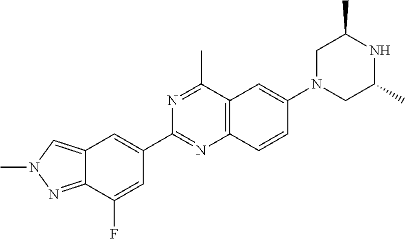 |
| 852 | 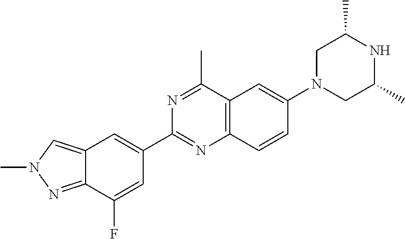 |
| 853 | 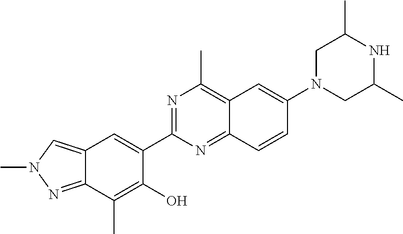 |
| 854 | 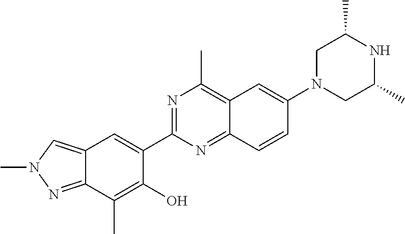 |
| 855 | 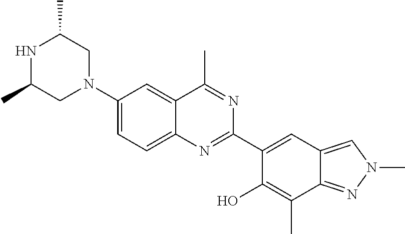 |
| 856 | 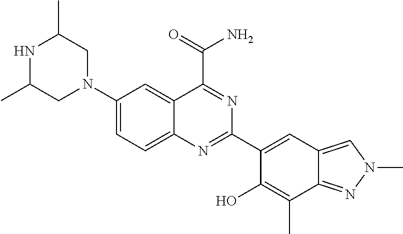 |
| 857 | 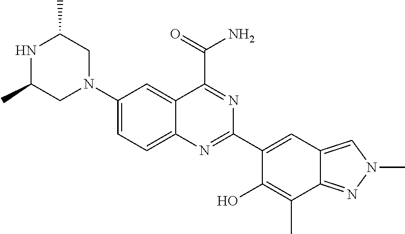 |
| 858 | 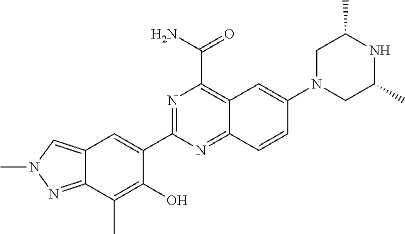 |
| 859 | 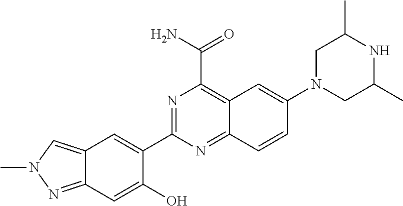 |
| 860 | 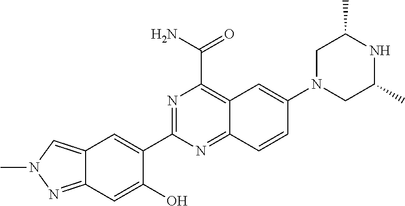 |
| 861 | 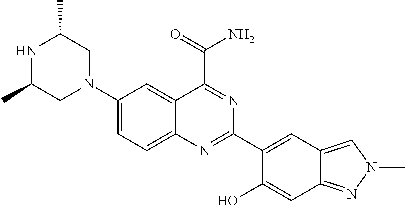 |
| 862 | 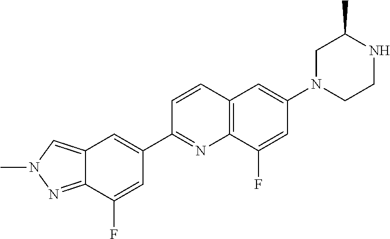 [SMILES: C[C@@H]1CN(c2cc(F)c3nc(-c4cc(F)c5nn(C)cc5c4)ccc3c2)CCN1] |
| 863 | 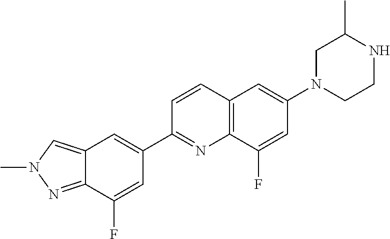 [SMILES: CC1CN(c2cc(F)c3nc(-c4cc(F)c5nn(C)cc5c4)ccc3c2)CCN1] |
| 864 | 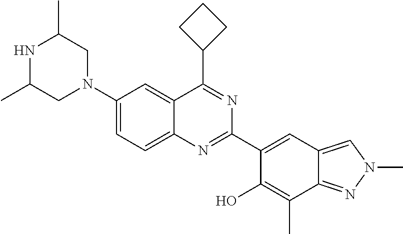 |
| 865 | 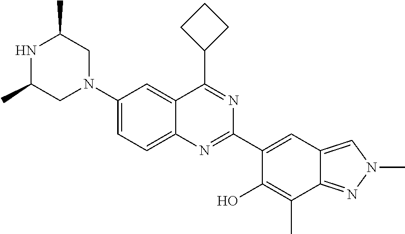 |
| 866 | 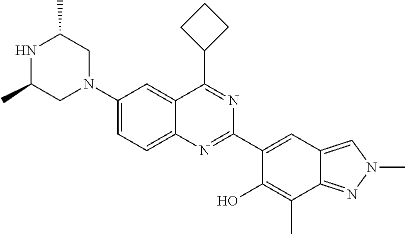 |
| 867 | 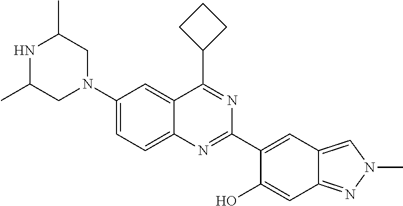 |
| 868 | 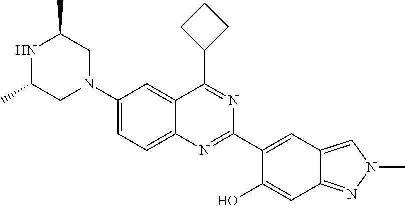 |
| 869 | 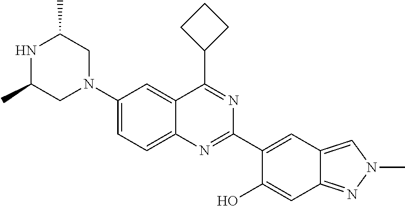 |
| 870 | 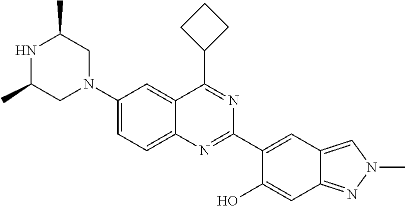 |
| 871 | 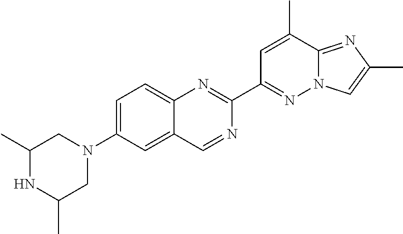 |
| 872 | 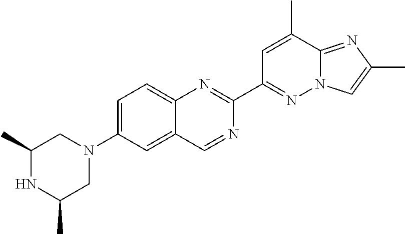 |
| 873 | 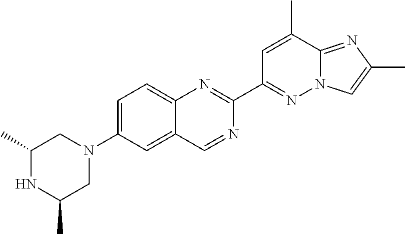 |
| 874 | 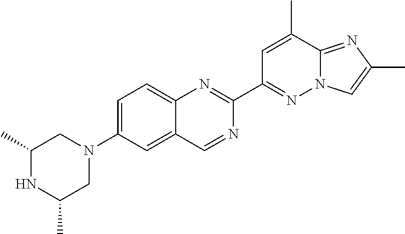 |
| 875 | 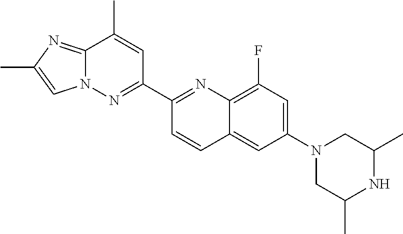 |
| 876 | 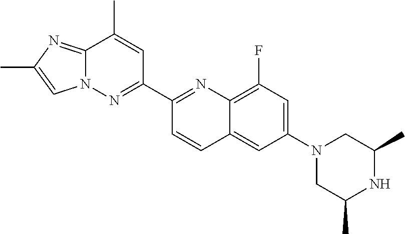 |
| 877 | 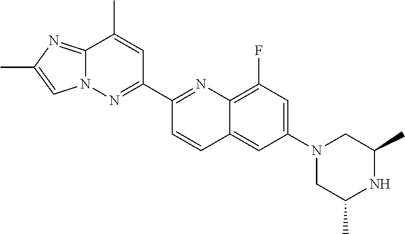 |
| 878 | 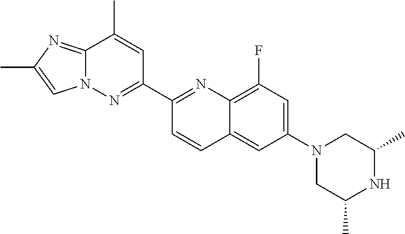 |
| 879 | 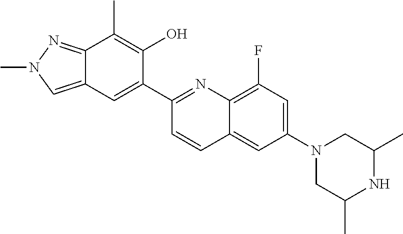 |
| 880 | 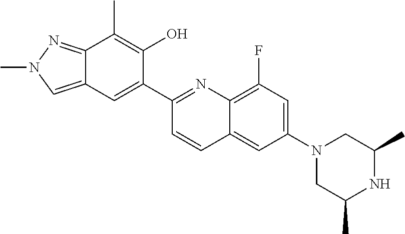 |
| 881 | 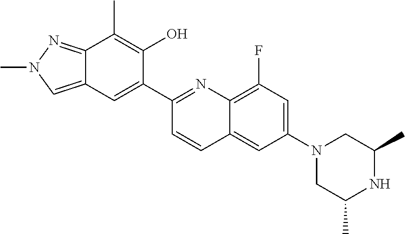 |
| 882 | 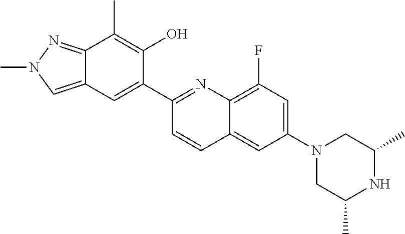 |
| 883 | 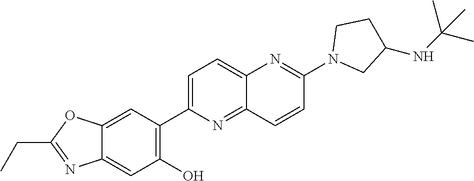 [SMILES: CCc1nc2cc(O)c(-c3ccc4nc(N5CCC(NC(C)(C)C)C5)ccc4n3)cc2o1] |
| 884 | 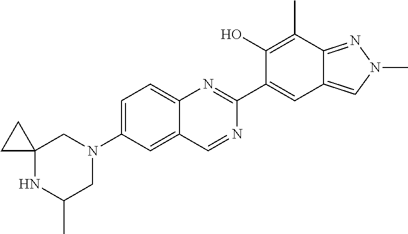 [SMILES: Cc1c(O)c(-c2ncc3cc(N4CC(C)NC5(CC5)C4)ccc3n2)cc2cn(C)nc12] |
| 885 | 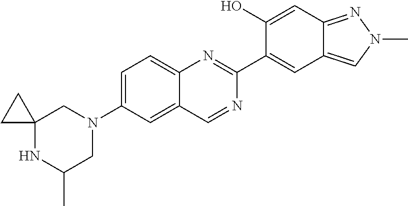 [SMILES: CC1CN(c2ccc3nc(-c4cc5cn(C)nc5cc4O)ncc3c2)CC2(CC2)N1] |
| 886 | 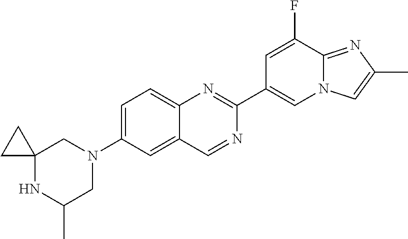 [SMILES: Cc1cn2cc(-c3ncc4cc(N5CC(C)NC6(CC6)C5)ccc4n3)cc(F)c2n1] |
| 887 | 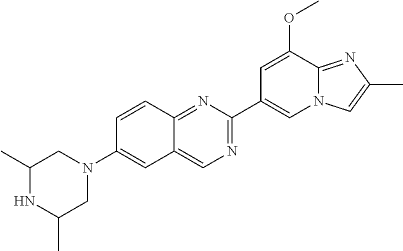 |
| 888 | 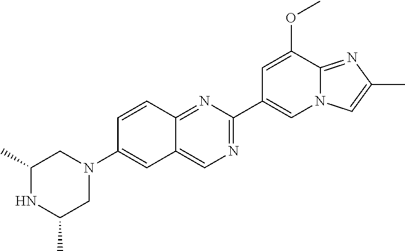 |
| 889 | 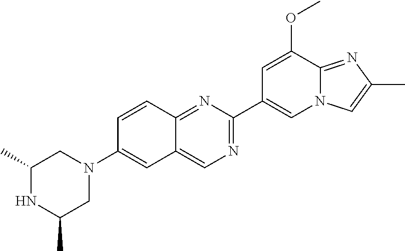 |
| 890 | 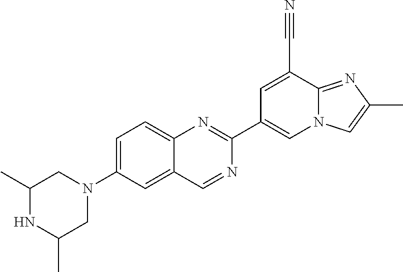 |
| 891 | 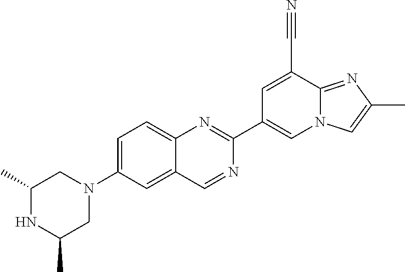 |
| 892 | 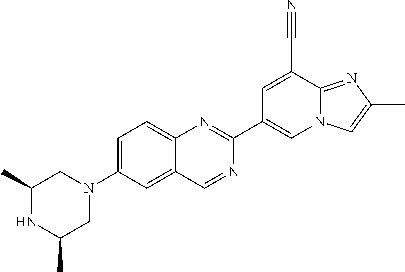 |
| 893 | 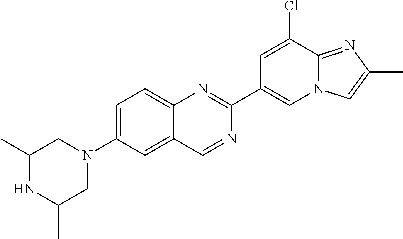 |
| 894 | 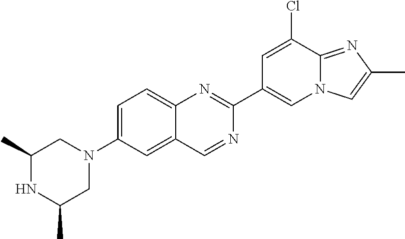 |
| 895 | 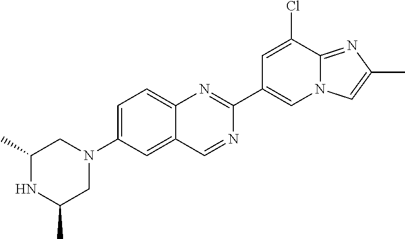 |
| 896 | 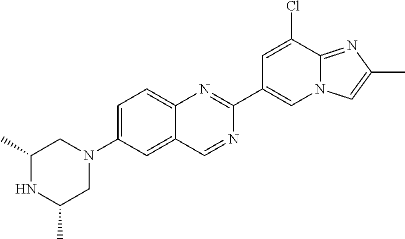 |
| 897 | 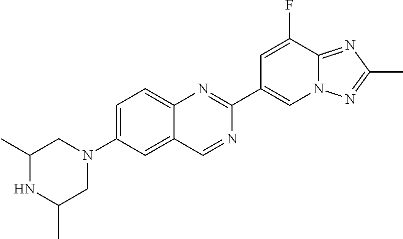 |
| 898 | 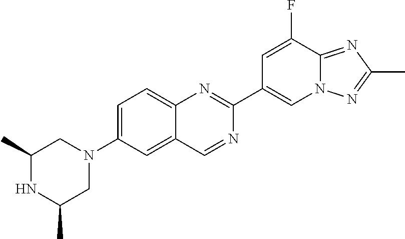 |
| 899 | 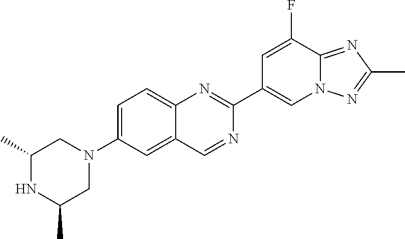 |
| 900 | 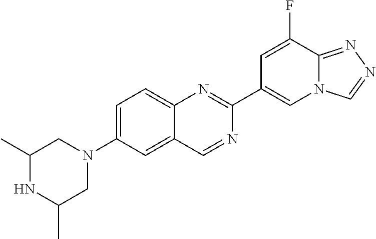 [SMILES: CC1CN(c2ccc3nc(-c4cc(F)c5nncn5c4)ncc3c2)CC(C)N1] |
| 901 | 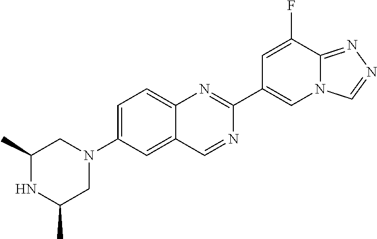 [SMILES: C[C@@H]1CN(c2ccc3nc(-c4cc(F)c5nncn5c4)ncc3c2)C[C@H](C)N1] |
| 902 | 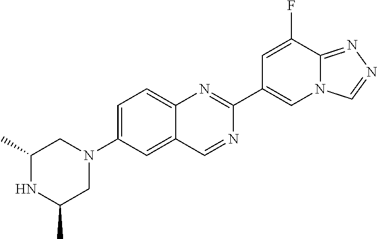 [SMILES: C[C@@H]1CN(c2ccc3nc(-c4cc(F)c5nncn5c4)ncc3c2)C[C@@H](C)N1] |
| 903 | 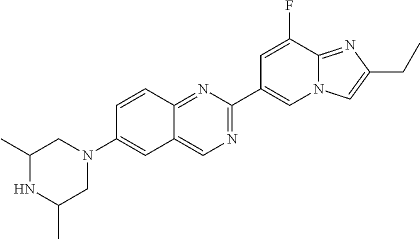 [SMILES: CCc1cn2cc(-c3ncc4cc(N5CC(C)NC(C)C5)ccc4n3)cc(F)c2n1] |
| 904 | 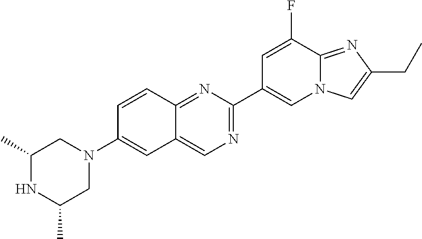 [SMILES: CCc1cn2cc(-c3ncc4cc(N5C[C@@H](C)N[C@@H](C)C5)ccc4n3)cc(F)c2n1] |
| 905 | 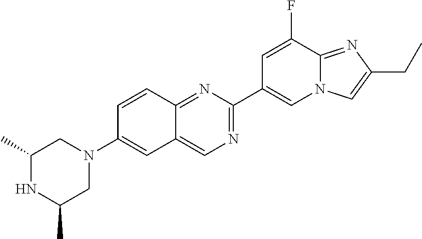 [SMILES: CCc1cn2cc(-c3ncc4cc(N5C[C@@H](C)N[C@H](C)C5)ccc4n3)cc(F)c2n1] |
| 906 | 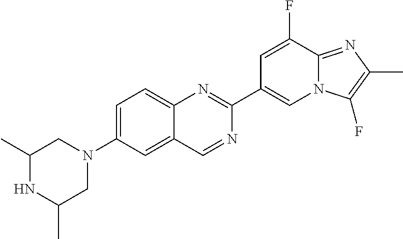 |
| 907 | 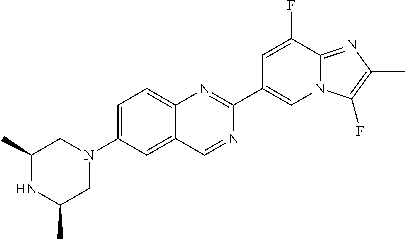 |
| 908 | 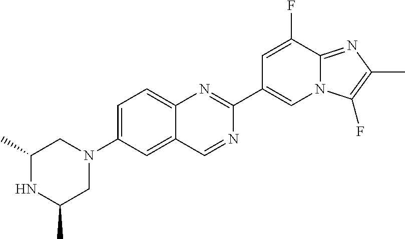 |
| 909 | 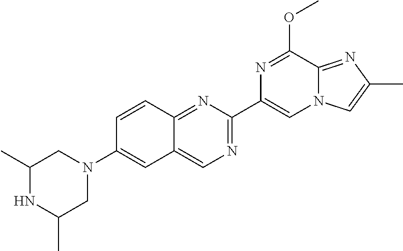 |
| 910 | 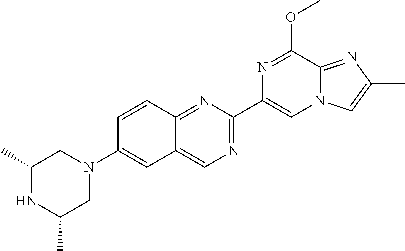 |
| 911 | 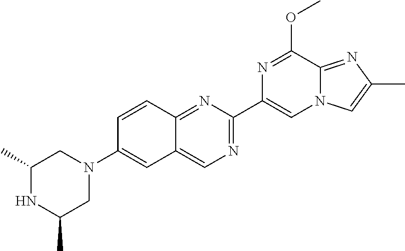 |
| 912 | 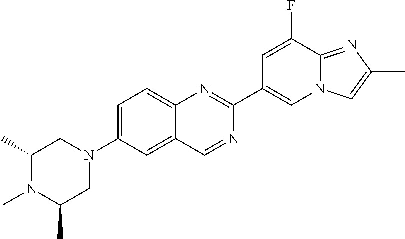 |
| 913 | 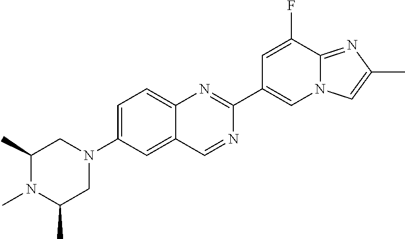 |
| 914 | 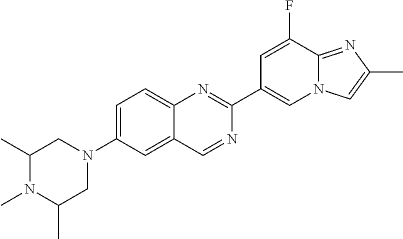 |
| 915 | 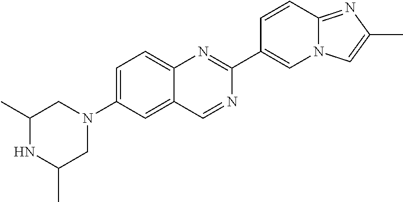 |
| 916 | 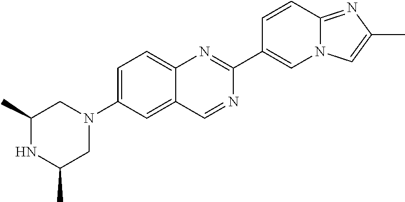 |
| 917 | 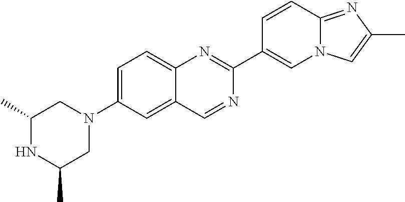 |
| 918 | 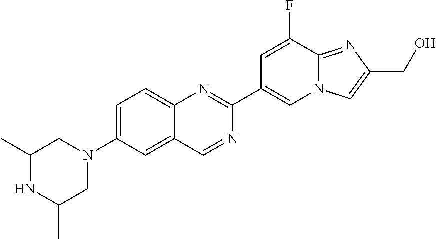 [SMILES: CC1CN(c2ccc3nc(-c4cc(F)c5nc(CO)cn5c4)ncc3c2)CC(C)N1] |
| 919 | 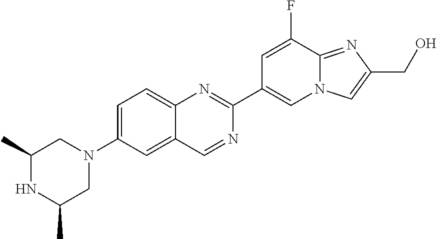 [SMILES: C[C@@H]1CN(c2ccc3nc(-c4cc(F)c5nc(CO)cn5c4)ncc3c2)C[C@H](C)N1] |
| 920 | 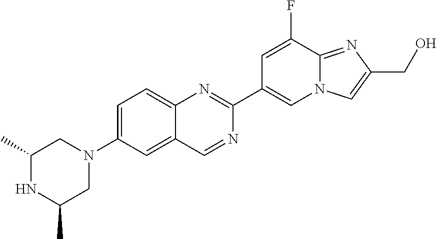 [SMILES: C[C@@H]1CN(c2ccc3nc(-c4cc(F)c5nc(CO)cn5c4)ncc3c2)C[C@@H](C)N1] |
| 921 | 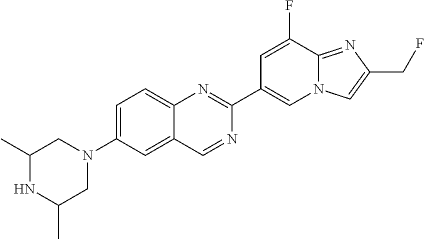 [SMILES: CC1CN(c2ccc3nc(-c4cc(F)c5nc(CF)cn5c4)ncc3c2)CC(C)N1] |
| 922 | 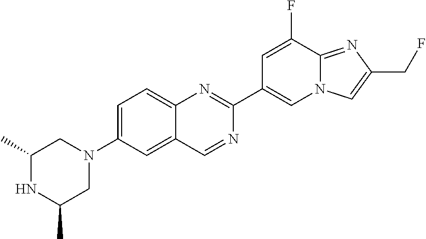 [SMILES: C[C@@H]1CN(c2ccc3nc(-c4cc(F)c5nc(CF)cn5c4)ncc3c2)C[C@@H](C)N1] |
| 923 | 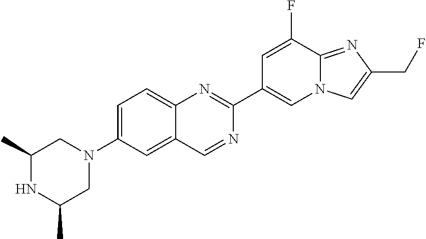 [SMILES: C[C@@H]1CN(c2ccc3nc(-c4cc(F)c5nc(CF)cn5c4)ncc3c2)C[C@H](C)N1] |
| 924 | 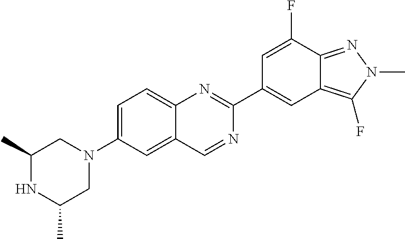 |
| 925 | 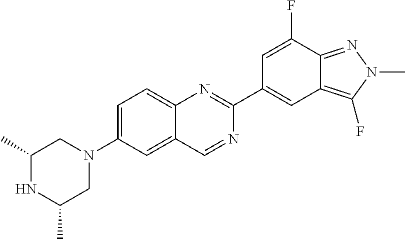 |
| 926 | 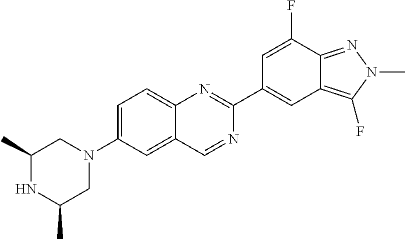 |
| 927 | 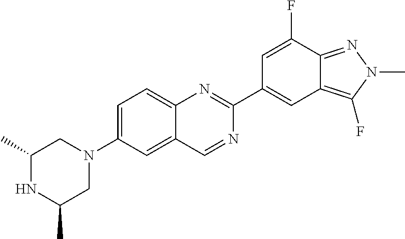 |
| 928 | 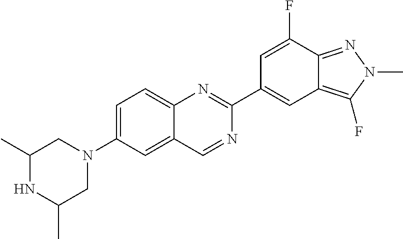 |
| 929 | 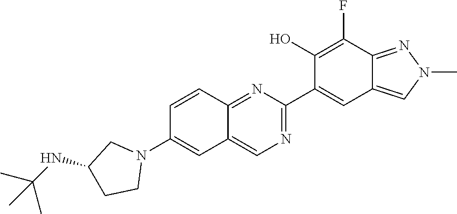 [SMILES: Cn1cc2cc(-c3ncc4cc(N5CC[C@H](NC(C)(C)C)C5)ccc4n3)c(O)c(F)c2n1] |
| 930 | 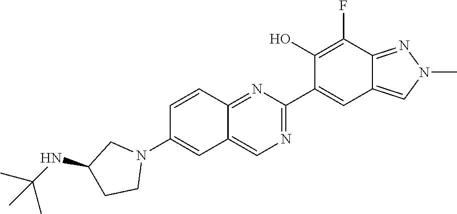 [SMILES: Cn1cc2cc(-c3ncc4cc(N5CC[C@@H](NC(C)(C)C)C5)ccc4n3)c(O)c(F)c2n1] |
| 931 | 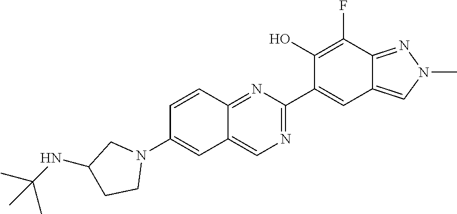 [SMILES: Cn1cc2cc(-c3ncc4cc(N5CCC(NC(C)(C)C)C5)ccc4n3)c(O)c(F)c2n1] |
| 932 | 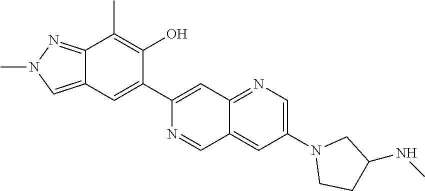 |
| 933 | 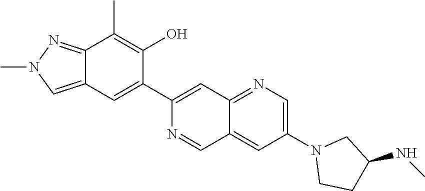 |
| 934 | 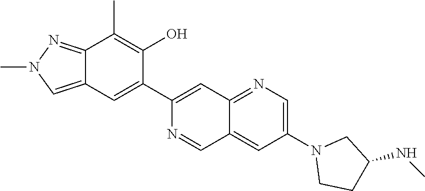 |
| 935 | 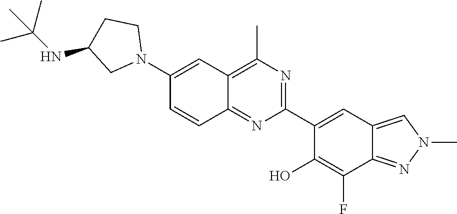 [SMILES: Cc1nc(-c2cc3cn(C)nc3c(F)c2O)nc2ccc(N3CC[C@H](NC(C)(C)C)C3)cc12] |
| 936 | 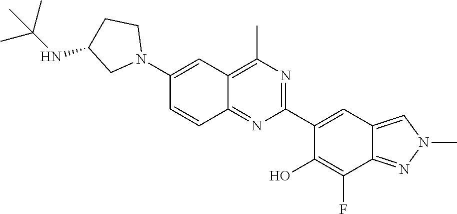 [SMILES: Cc1nc(-c2cc3cn(C)nc3c(F)c2O)nc2ccc(N3CC[C@@H](NC(C)(C)C)C3)cc12] |
| 937 | 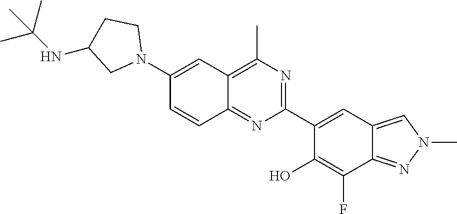 [SMILES: Cc1nc(-c2cc3cn(C)nc3c(F)c2O)nc2ccc(N3CCC(NC(C)(C)C)C3)cc12] |
| 938 | 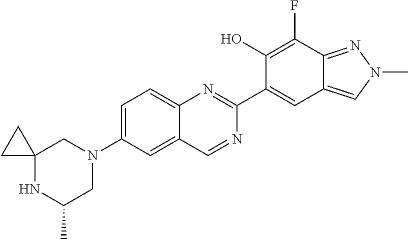 [SMILES: C[C@H]1CN(c2ccc3nc(-c4cc5cn(C)nc5c(F)c4O)ncc3c2)CC2(CC2)N1] |
| 939 | 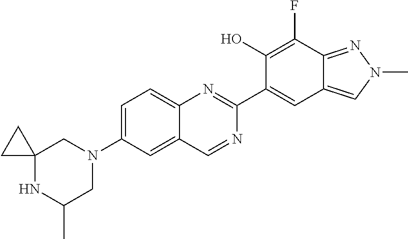 [SMILES: CC1CN(c2ccc3nc(-c4cc5cn(C)nc5c(F)c4O)ncc3c2)CC2(CC2)N1] |
| 940 | 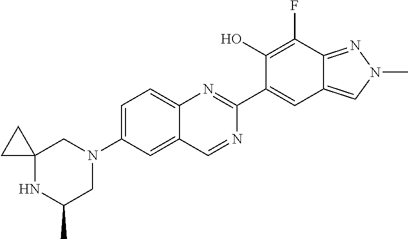 [SMILES: C[C@@H]1CN(c2ccc3nc(-c4cc5cn(C)nc5c(F)c4O)ncc3c2)CC2(CC2)N1] |
| 941 | 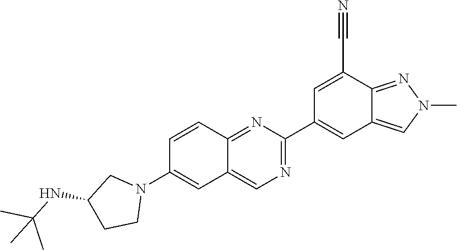 [SMILES: Cn1cc2cc(-c3ncc4cc(N5CC[C@H](NC(C)(C)C)C5)ccc4n3)cc(C#N)c2n1] |
| 942 | 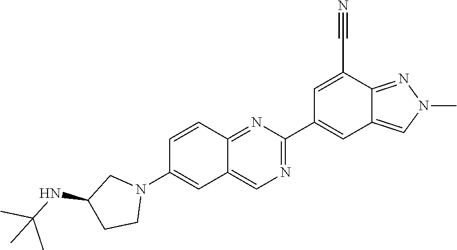 [SMILES: Cn1cc2cc(-c3ncc4cc(N5CC[C@@H](NC(C)(C)C)C5)ccc4n3)cc(C#N)c2n1] |
| 943 | 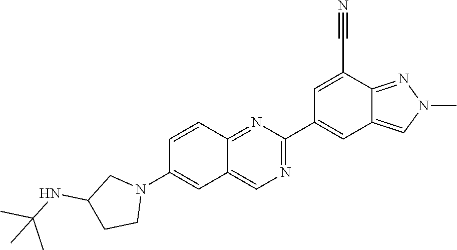 [SMILES: Cn1cc2cc(-c3ncc4cc(N5CCC(NC(C)(C)C)C5)ccc4n3)cc(C#N)c2n1] |
| 944 | 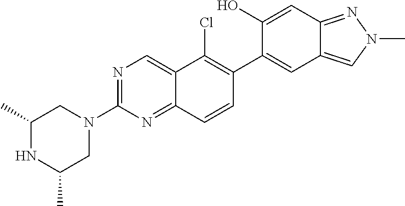 |
| 945 | 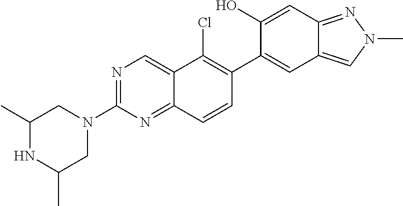 |
| 946 | 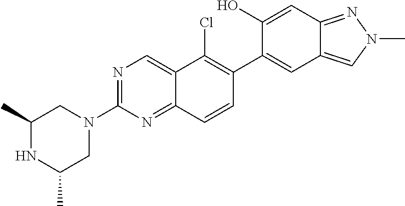 |
| 947 | 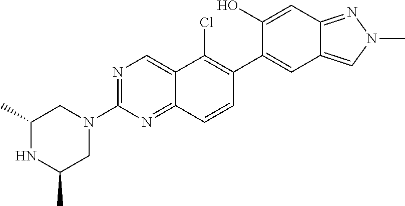 |
| 948 | 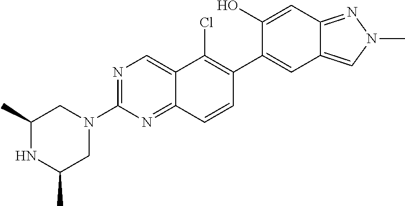 |
| 949 | 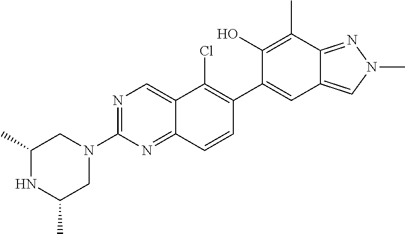 |
| 950 | 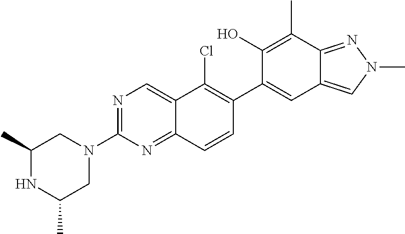 |
| 951 | 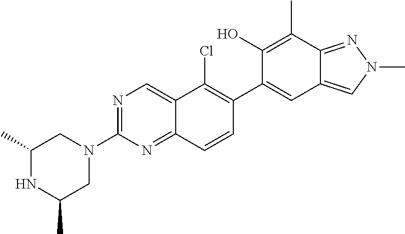 |
| 952 | 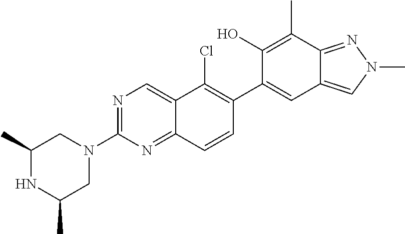 |
| 953 | 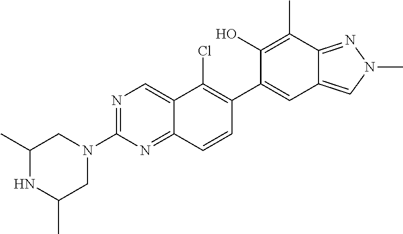 |
| 954 | 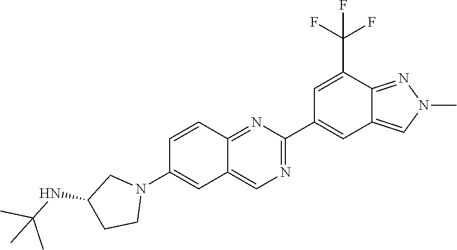 [SMILES: Cn1cc2cc(-c3ncc4cc(N5CC[C@H](NC(C)(C)C)C5)ccc4n3)cc(C(F)(F)F)c2n1] |
| 955 | 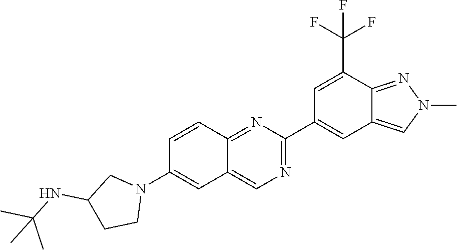 [SMILES: Cn1cc2cc(-c3ncc4cc(N5CCC(NC(C)(C)C)C5)ccc4n3)cc(C(F)(F)F)c2n1] |
| 956 | 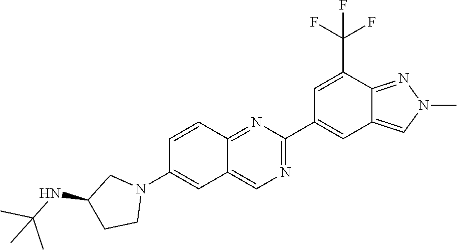 [SMILES: Cn1cc2cc(-c3ncc4cc(N5CC[C@@H](NC(C)(C)C)C5)ccc4n3)cc(C(F)(F)F)c2n1] |
| 957 | 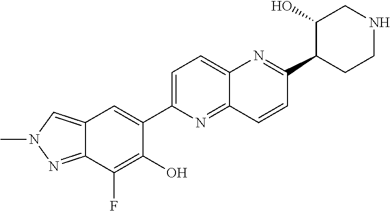 [SMILES: Cn1cc2cc(-c3ccc4nc([C@@H]5CCNC[C@H]5O)ccc4n3)c(O)c(F)c2n1] |
| 958 | 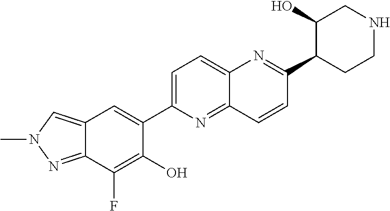 [SMILES: Cn1cc2cc(-c3ccc4nc([C@@H]5CCNC[C@@H]5O)ccc4n3)c(O)c(F)c2n1] |
| 959 | 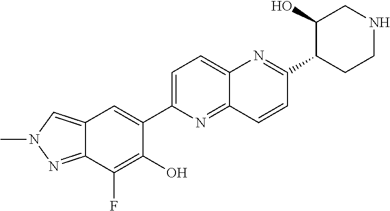 [SMILES: Cn1cc2cc(-c3ccc4nc([C@H]5CCNC[C@@H]5O)ccc4n3)c(O)c(F)c2n1] |
| 960 | 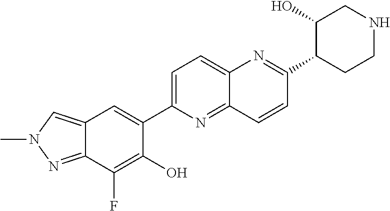 [SMILES: Cn1cc2cc(-c3ccc4nc([C@H]5CCNC[C@H]5O)ccc4n3)c(O)c(F)c2n1] |
| 961 | 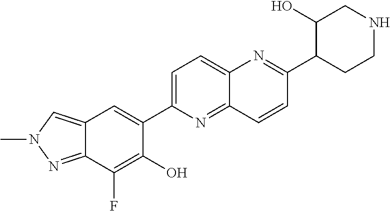 [SMILES: Cn1cc2cc(-c3ccc4nc(C5CCNCC5O)ccc4n3)c(O)c(F)c2n1] |
| 962 | 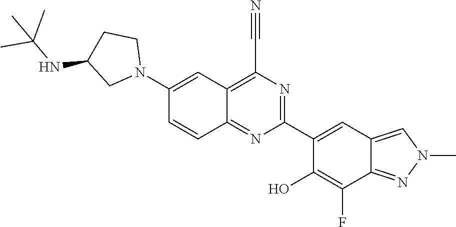 [SMILES: Cn1cc2cc(-c3nc(C#N)c4cc(N5CC[C@H](NC(C)(C)C)C5)ccc4n3)c(O)c(F)c2n1] |
| 963 | 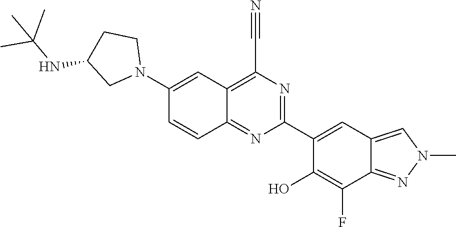 [SMILES: Cn1cc2cc(-c3nc(C#N)c4cc(N5CC[C@@H](NC(C)(C)C)C5)ccc4n3)c(O)c(F)c2n1] |
| 964 | 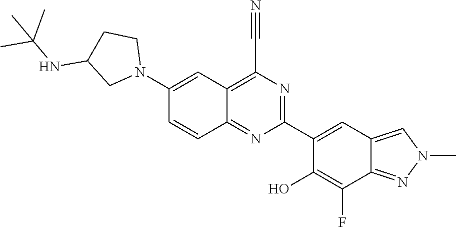 [SMILES: Cn1cc2cc(-c3nc(C#N)c4cc(N5CCC(NC(C)(C)C)C5)ccc4n3)c(O)c(F)c2n1] |

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 100, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 101, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 102, or a pharmaceutically acceptable salt, solvate, hydrate, tautomer, or stereoisomer thereof.

In some embodiments, for Formula (I), A is bicyclic heteroaryl (e.g., 7-hydroxy-2-methylimidazo[1,2-b]pyridazinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 103, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X and Z are N; Y is C(R^{5a}) (e.g., —CH—); and m and n are 0. In some embodiments, the compound of Formula (I) is Compound 104, or a pharmaceutically acceptable salt, solvate, hydrate, tautomer, or stereoisomer thereof.

In some embodiments, for Formula (I), A is bicyclic heteroaryl (e.g., 4-hydroxy-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 105, or a pharmaceutically acceptable salt, solvate, hydrate, tautomer, or stereoisomer thereof.

In some embodiments, for Formula (I), A is bicyclic heteroaryl (e.g., 4-hydroxy-2-methyl-2H-indazolyl); B is monocyclic heterocyclyl (e.g., piperidinyl); L¹ and L² are absent; X and Z are N; Y is C(R^{5a}) (e.g., —CH—); and m and n are 0. In some embodiments, the compound of Formula (I) is Compound 106, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 107, or a pharmaceutically acceptable salt, solvate, hydrate, tautomer, or stereoisomer thereof.

In some embodiments, for Formula (I), A is bicyclic heteroaryl (e.g., 4-hydroxy-2-methyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 108, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X and Y are independently C(R^{5a}) (e.g., —CH—); Z is N; and m and n are 0. In some embodiments, the compound of Formula (I) is Compound 109, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is C(R^{5a}) (e.g., —CH—); Y and Z are N; R² is halo (e.g., fluoro); m is 1; and n is 0. In some embodiments, the compound of Formula (I) is Compound 113, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is C(R^{5a}) (e.g., —CH—); Y and Z are N; R³ is halo (e.g., fluoro); m is 0; and n is 1. In some embodiments, the compound of Formula (I) is Compound 114, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is C(R^{5a}) (e.g., —CH—); Y is C(R^{5a}) (e.g., —CF—); Z is N; and m and n are 0. In some embodiments, the compound of Formula (I) is Compound 115, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-methyl-piperazyl); L¹ and L² are absent; X is C(R^{5a}) (e.g., —CH—); Y and Z are N; and m and n are 0. In some embodiments, the compound of Formula (I) is Compound 116, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2-methylpiperazyl); L¹ and L² are absent; X is C(R^{5a}) (e.g., —CH—); Y and Z are N; and m and n are 0. In some embodiments, the compound of Formula (I) is Compound 117, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-methyl-2-methylpiperazyl); L¹ and L² are absent; X is C(R^{5a}) (e.g., —CH—); Y and Z are N; and m and n are 0. In some embodiments, the compound of Formula (I) is Compound 118, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2-ethylpiperazyl); L¹ and L² are absent; X is C(R^{5a}) (e.g., —CH—); Y and Z are N; and m and n are 0. In some embodiments, the compound of Formula (I) is Compound 119, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,2-dimethylpiperazyl); L¹ and L² are absent; X is C(R^{5a}) (e.g., —CH—); Y and Z are N; and m and n are 0. In some embodiments, the compound of Formula (I) is Compound 120, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 4,7-diazaspiro[2.5]octanyl); L¹ and L² are absent; X is C(R^{5a}) (e.g., —CH—); Y and Z are N; and m and n are 0. In some embodiments, the compound of Formula (I) is Compound 121, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,2,6,6-tetramethylpiperazyl); L¹ and L² are absent; X is C(R^{5a}) (e.g., —CH—); Y and Z are N; and m and n are 0. In some embodiments, the compound of Formula (I) is Compound 122, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 2-methyl-2,6-diazaspiro[3.3]heptanyl); L¹ and L² are absent; X is C(R^{5a}) (e.g., —CH—); Y and Z are N; and m and n are 0. In some embodiments, the compound of Formula (I) is Compound 123, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 2,6-diazaspiro[3.3]heptanyl); L¹ and L² are absent; X is C(R^{5a}) (e.g., —CH—); Y and Z are N; and m and n are 0. In some embodiments, the compound of Formula (I) is Compound 124, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1,3-bipyrrolidinyl); L¹ and L² are absent; X is C(R^{5a}) (e.g., —CH—); Y and Z are N; and m and n are 0. In some embodiments, the compound of Formula (I) is Compound 125, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-methylpiperidinyl); L¹ is absent; L² is —N(R⁴)— (e.g., —N(CH₃)—); X is C(R^{5a}) (e.g., —CH—); Y and Z are N; and m and n are 0. In some embodiments, the compound of Formula (I) is Compound 126, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 127, 153, 154, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N,N-(dimethyl)amino)pyrrolidinyl); L¹ and L² are absent; X is C(R^{5a}) (e.g., —CH—); Y and Z are N; and m and n are 0. In some embodiments, the compound of Formula (I) is Compound 129, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,2,6,6-tetramethylpiperidinyl); L¹ is absent; L² is —N(R⁴)— (e.g., —N(CH₃)—); X is C(R^{5a}) (e.g., —CH—); Y and Z are N; and m and n are 0. In some embodiments, the compound of Formula (I) is Compound 130, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 4-(N-(ethyl)amino)piperidinyl); L¹ and L² are absent; X is C(R^{5a}) (e.g., —CH—); Y and Z are N; and m and n are 0. In some embodiments, the compound of Formula (I) is Compound 131, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 4-(N-(tert-butyl)amino)piperidinyl); L¹ and L² are absent; X is C(R^{5a}) (e.g., —CH—); Y and Z are N; and m and n are 0. In some embodiments, the compound of Formula (I) is Compound 132, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 4-(N,N-(dimethyl)amino)piperidinyl); L¹ and L² are absent; X is C(R^{5a}) (e.g., —CH—); Y and Z are N; and m and n are 0. In some embodiments, the compound of Formula (I) is Compound 133, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., octahydropyrrolo[1,2-a]pyrazinyl); L¹ and L² are absent; X is C(R^{5a}) (e.g., —CH—); Y and Z are N; and m and n are 0. In some embodiments, the compound of Formula (I) is Compound 134, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 3,6-diazabicyclo[3.1.1]heptanyl); L¹ and L² are absent; X is C(R^{5a}) (e.g., —CH—); Y and Z are N; and m and n are 0. In some embodiments, the compound of Formula (I) is Compound 135, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperazyl); L¹ and L² are absent; X is C(R^{5a}) (e.g., —CH—); Y and Z are N; and m and n are 0. In some embodiments, the compound of Formula (I) is Compound 136, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperidinyl); L¹ and L² are absent; X is C(R^{5a}) (e.g., —CH—); Y and Z are N; and m and n are 0. In some embodiments, the compound of Formula (I) is Compound 137, 545, 546, 547, 548, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-ethylpiperidinyl); L¹ and L² are absent; X is C(R^{5a}) (e.g., —CH—); Y and Z are N; and m and n are 0. In some embodiments, the compound of Formula (I) is Compound 138, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2-methylpiperidinyl); L¹ and L² are absent; X is C(R^{5a}) (e.g., —CH—); Y and Z are N; and m and n are 0. In some embodiments, the compound of Formula (I) is Compound 139, 140, 549, 550, 551, or a pharmaceutically acceptable salt, solvate, hydrate, tautomer, or stereoisomer thereof.

In some embodiments, for Formula (I), A is bicyclic heteroaryl (e.g., 4-hydroxy-7-bromo-2-methyl-2H-indazolyl); B is monocyclic heterocyclyl (e.g., piperidinyl); L¹ and L² are absent; Y is C(R^{5a}) (e.g., —CH—); X and Z are N; and m and n are 0. In some embodiments, the compound of Formula (I) is Compound 141, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., N-methylpiperidinyl); L¹ and L² are absent; X is C(R^{5a}) (e.g., —CH—); Y and Z are N; and m and n are 0. In some embodiments, the compound of Formula (I) is Compound 142, or a pharmaceutically acceptable salt, solvate, hydrate, tautomer, or stereoisomer thereof.

In some embodiments, for Formula (I), A is bicyclic heteroaryl (e.g., 6-methoxy-2-methyl-2H-indazolyl); B is monocyclic heterocyclyl (e.g., piperidinyl); L¹ and L² are absent; X is C(R^{5a}) (e.g., —CH—); Y is C(R^{5a}) (e.g., —CF—); Z is N; and m and n are 0. In some embodiments, the compound of Formula (I) is Compound 143, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 144, or a pharmaceutically acceptable salt, solvate, hydrate, tautomer, or stereoisomer thereof.

In some embodiments, for Formula (I), A is bicyclic heteroaryl (e.g., 6-methoxy-2-methyl-2H-indazolyl); B is monocyclic heterocyclyl (e.g., piperidinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 145, or a pharmaceutically acceptable salt, solvate, hydrate, tautomer, or stereoisomer thereof.

In some embodiments, for Formula (I), A is bicyclic heteroaryl (e.g., 5-hydroxy-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 146, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 147, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]thiazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 148, or a pharmaceutically acceptable salt, solvate, hydrate, tautomer, or stereoisomer thereof.

In some embodiments, for Formula (I), A is bicyclic heteroaryl (e.g., 7-hydroxy-2-methylimidazo[1,2-a]pyridinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 149, or a pharmaceutically acceptable salt, solvate, hydrate, tautomer, or stereoisomer thereof.

In some embodiments, for Formula (I), A is bicyclic heteroaryl (e.g., 3-hydroxy-4,6-dimethylpyrazolo[1,5-a]pyrazinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 150, or a pharmaceutically acceptable salt, solvate, hydrate, tautomer, or stereoisomer thereof.

In some embodiments, for Formula (I), A is bicyclic heteroaryl (e.g., 7-hydroxy-2,8-dimethylimidazo[1,2-a]pyridinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 151, or a pharmaceutically acceptable salt, solvate, hydrate, tautomer, or stereoisomer thereof.

In some embodiments, for Formula (I), A is monocyclic heteroaryl (e.g., 1H-imidazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 152, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 155, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 156, 157, 262, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 158, 197, 198, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., azetidinyl); L¹ is absent; L² is —O—; X is C(R^{5a}) (e.g., —CH—); Y and Z are N; and m and n are 0. In some embodiments, the compound of Formula (I) is Compound 159, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is tricyclic heterocyclyl (e.g., octahydro-1H-6λ²-cyclopenta[2,1-b:5,1-b]dipyrrolyl); L¹ and L² are absent; X is C(R^{5a}) (e.g., —CH—); Y and Z are N; and m and n are 0.

In some embodiments, the compound of Formula (I) is Compound 160, or a pharmaceutically acceptable salt, solvate, hydrate, tautomer, or stereoisomer thereof.

In some embodiments, for Formula (I), A is bicyclic heteroaryl (e.g., 6-methoxyisoquinolinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 161, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1,6-diazaspiro[3.4]octanyl); L¹ and L² are absent; X is C(R^{5a}) (e.g., —CH—); Y and Z are N; and m and n are 0. In some embodiments, the compound of Formula (I) is Compound 162, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1,6-diazaspiro[3.5]nonanyl); L¹ and L² are absent; X is C(R^{5a}) (e.g., —CH—); Y and Z are N; and m and n are 0. In some embodiments, the compound of Formula (I) is Compound 163, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1,7-diazaspiro[3.5]nonanyl); L¹ and L² are absent; X is C(R^{5a}) (e.g., —CH—); Y and Z are N; and m and n are 0. In some embodiments, the compound of Formula (I) is Compound 164, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(tert-butyl)amino)azetidinyl); L¹ and L² are absent; X is C(R^{5a}) (e.g., —CH—); Y and Z are N; and m and n are 0. In some embodiments, the compound of Formula (I) is Compound 165, or a pharmaceutically acceptable salt, solvate, hydrate, tautomer, or stereoisomer thereof.

In some embodiments, for Formula (I), A is bicyclic heteroaryl (e.g., 6-hydroxy-isoquinolinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 166, or a pharmaceutically acceptable salt, solvate, hydrate, tautomer, or stereoisomer thereof.

In some embodiments, for Formula (I), A is bicyclic heteroaryl (e.g., 6-hydroxy-3-methylquinazolin-4(3H)-onyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 167, or a pharmaceutically acceptable salt, solvate, hydrate, tautomer, or stereoisomer thereof.

In some embodiments, for Formula (I), A is bicyclic heteroaryl (e.g., 6-methoxy-3-methylquinazolin-4(3H)-onyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 168, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 7-methyl-1,7-diazaspiro[3.5]nonanyl); L¹ and L² are absent; X is C(R^{5a}) (e.g., —CH—); Y and Z are N; and m and n are 0. In some embodiments, the compound of Formula (I) is Compound 169, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(methyl)amino)pyrrolidinyl); L¹ and L² are absent; X is C(R^{5a}) (e.g., —CH—); Y and Z are N; and m and n are 0. In some embodiments, the compound of Formula (I) is Compound 170, 172, 263, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 171, 173, 264, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-4H-chromen-4-onyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 174, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 175, 209, 210, or a pharmaceutically acceptable salt, solvate, hydrate, tautomer, or stereoisomer thereof.

In some embodiments, for Formula (I), A is bicyclic heteroaryl (e.g., 5-methoxy-2-methylbenzo[d]thiazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 176, or a pharmaceutically acceptable salt, solvate, hydrate, tautomer, or stereoisomer thereof.

In some embodiments, for Formula (I), A is bicyclic heteroaryl (e.g., 5-methoxy-2-methylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 177, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1,6-diazaspiro[3.4]octanyl); L¹ and L² are absent; X is C(R^{5a}) (e.g., —CH—); Y and Z are N; and m and n are 0. In some embodiments, the compound of Formula (I) is Compound 178, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(ethyl)amino)pyrrolidinyl); L¹ and L² are absent; X is C(R^{5a}) (e.g., —CH—); Y and Z are N; and m and n are 0. In some embodiments, the compound of Formula (I) is Compound 179, 180, 265, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 1-methyl-1,6-diazaspiro[3.4]octanyl); L¹ and L² are absent; X is C(R^{5a}) (e.g., —CH—); Y and Z are N; and m and n are 0. In some embodiments, the compound of Formula (I) is Compound 181, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(2,2,2-trifluoroethyl)amino)pyrrolidinyl); L¹ and L² are absent; X is C(R^{5a}) (e.g., —CH—); Y and Z are N; and m and n are 0. In some embodiments, the compound of Formula (I) is Compound 182, 245, 266, or a pharmaceutically acceptable salt, solvate, hydrate, tautomer, or stereoisomer thereof.

In some embodiments, for Formula (I), A is bicyclic heteroaryl (e.g., 6-fluoro-2-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 183, or a pharmaceutically acceptable salt, solvate, hydrate, tautomer, or stereoisomer thereof.

In some embodiments, for Formula (I), A is bicyclic heteroaryl (e.g., 2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 184, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 185, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 186, or a pharmaceutically acceptable salt, solvate, hydrate, tautomer, or stereoisomer thereof.

In some embodiments, for Formula (I), A is bicyclic heteroaryl (e.g., 4,6-dimethylpyrazolo[1,5-a]pyrazinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 187, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(oxetanyl)amino)pyrrolidinyl); L¹ and L² are absent; X is C(R^{5a}) (e.g., —CH—); Y and Z are N; and m and n are 0. In some embodiments, the compound of Formula (I) is Compound 188, 189, 267, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]thiazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 190, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 191, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 6-methyl-1,6-diazaspiro[3.5]nonanyl); L¹ and L² are absent; X is C(R^{5a}) (e.g., —CH—); Y and Z are N; and m and n are 0. In some embodiments, the compound of Formula (I) is Compound 192, or a pharmaceutically acceptable salt, solvate, hydrate, tautomer, or stereoisomer thereof.

In some embodiments, for Formula (I), A is bicyclic heteroaryl (e.g., 6-fluoro-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 193, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 194, 195, 268, or a pharmaceutically acceptable salt, solvate, hydrate, tautomer, or stereoisomer thereof.

In some embodiments, for Formula (I), A is bicyclic heteroaryl (e.g., 6-(difluoromethyl)-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 196, or a pharmaceutically acceptable salt, solvate, hydrate, tautomer, or stereoisomer thereof.

In some embodiments, for Formula (I), A is bicyclic heteroaryl (e.g., 2-methyl-2H-pyrazolo[4,3-b]pyridinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 199, or a pharmaceutically acceptable salt, solvate, hydrate, tautomer, or stereoisomer thereof.

In some embodiments, for Formula (I), A is bicyclic heteroaryl (e.g., 2-methyl-2H-pyrazolo[3,4-c]pyridinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 200, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 201, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 202, or a pharmaceutically acceptable salt, solvate, hydrate, tautomer, or stereoisomer thereof.

In some embodiments, for Formula (I), A is bicyclic heteroaryl (e.g., 3-hydroxy-4,6-dimethylpyrazolo[1,5-a]pyrazinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 203, or a pharmaceutically acceptable salt, solvate, hydrate, tautomer, or stereoisomer thereof.

In some embodiments, for Formula (I), A is bicyclic heteroaryl (e.g., 6-cyano-2-methyl-2H-indazolyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 204, or a pharmaceutically acceptable salt, solvate, hydrate, tautomer, or stereoisomer thereof.

In some embodiments, for Formula (I), A is bicyclic heteroaryl (e.g., 6-fluoro-2-methyl-2H-pyrazolo[4,3-b]pyridinyl); B is monocyclic heterocyclyl (e.g., piperidinyl); L¹ and L² are absent; X is C(R^{5a}) (e.g., —CH—); Y and Z are N; and m and n are 0. In some embodiments, the compound of Formula (I) is Compound 205, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyrazinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 206, or a pharmaceutically acceptable salt, solvate, hydrate, tautomer, or stereoisomer thereof.

In some embodiments, for Formula (I), A is bicyclic heteroaryl (e.g., 6-fluoro-2-methyl-2H-pyrazolo[4,3-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 207, or a pharmaceutically acceptable salt, solvate, hydrate, tautomer, or stereoisomer thereof.

In some embodiments, for Formula (I), A is bicyclic heteroaryl (e.g., 2,7-dimethyl-2H-pyrazolo[3,4-c]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 208, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 211, 212, 271, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is 214, 215, 272, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 216, 217, 273, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(tert-butyl)amino)pyrrolidinyl); L¹ and L² are absent; Y is C(R^{5a}) (e.g., —CH—); X and Z are N; and m and n are 0. In some embodiments, the compound of Formula (I) is Compound 218, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 219, 220, 221, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 222, 223, 274, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 224, 225, 226, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 228, 229, 230, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 231, 232, 233, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-aminopyrrolidinyl); L¹ and L² are absent; X is C(R^{5a}) (e.g., —CH—); Y and Z are N; and m and n are 0. In some embodiments, the compound of Formula (I) is Compound 234, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is 235, 236, 237, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-aminopyrrolidinyl); L¹ and L² are absent; X is C(R^{5a}) (e.g., —CH—); Y and Z are N; and m and n are 0. In some embodiments, the compound of Formula (I) is Compound 238, 362, 363, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., piperidinyl); L¹ and L² are absent; X and Y are each independently C(R^{5a}) (e.g., —CH—); Z is N; and m and n are 0. In some embodiments, the compound of Formula (I) is Compound 241, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 246, 247, 277, 299, 300, 552, 609, 610, 611, 612, or a pharmaceutically acceptable salt, solvate, hydrate, tautomer, or stereoisomer thereof.

In some embodiments, for Formula (I), A is bicyclic heteroaryl (e.g., 2,7-dimethyl-2H-pyrazolo[4,3-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 248, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 249, 250, or a pharmaceutically acceptable salt, solvate, hydrate, tautomer, or stereoisomer thereof.

In some embodiments, for Formula (I), A is bicyclic heteroaryl (e.g., 6-methoxy-7-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 251, or a pharmaceutically acceptable salt, solvate, hydrate, tautomer, or stereoisomer thereof.

In some embodiments, for Formula (I), A is bicyclic heteroaryl (e.g., 3-hydroxy-4,6-dimethylpyrazolo[1,5-a]pyrazinyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 252, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 253, 254, 278, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 255, 256, 279, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 257, 258, 280, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 259, 260, 281, or a pharmaceutically acceptable salt, solvate, hydrate, tautomer, or stereoisomer thereof.

In some embodiments, for Formula (I), A is bicyclic heteroaryl (e.g., 6-hydroxy-7-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 282, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methyl)amino)pyrrolidinyl); L¹ and L² are absent; Y is C(R^{5a}) (e.g., —CH—); X and Z are N; and m and n are 0. In some embodiments, the compound of Formula (I) is Compound 283, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 284, 321, 322, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 285, 286, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 287, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 288, 289, 290, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 291, 292, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 293, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 294, 323, 324, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 295, 296, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(bicyclo[1.1.1]pentan-1-yl)amino)pyrrolidinyl); L¹ and L² are absent; X is C(R^{5a}) (e.g., —CH—); Y and Z are N; and m and n are 0. In some embodiments, the compound of Formula (I) is Compound 297, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-pyrazolo[4,3-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 298, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperidinyl); L¹ and L² are absent; Y is C(R^{5a}) (e.g., —CH—); X and Z are N; and m and n are 0. In some embodiments, the compound of Formula (I) is Compound 301, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(oxetanyl)amino)pyrrolidinyl); L¹ and L² are absent; X is C(R^{5a}) (e.g., —CH—); Y and Z are N; and m and n are 0. In some embodiments, the compound of Formula (I) is Compound 302, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 303, 304, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 305, 306, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 307, 308, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is 309, 310, 311, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is 312, 313, 314, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(cyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 315, 316, 317, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 318, 319, 320, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 325, 326, 327, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 328, 329, 330, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., 3-(N-(methyl)amino)pyrrolidinyl); L¹ and L² are absent; X, Y, and Z are N; and m and n are 0. In some embodiments, the compound of Formula (I) is Compound 331, 332, 393, or a pharmaceutically acceptable salt, solvate, hydrate, tautomer, or stereoisomer thereof.

In some embodiments, for Formula (I), A is bicyclic heteroaryl (e.g., 5-hydroxy-2-cyclopropylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 333, or a pharmaceutically acceptable salt, solvate, hydrate, tautomer, or stereoisomer thereof.

In some embodiments, for Formula (I), A is bicyclic heteroaryl (e.g., 5-hydroxy-2-(methoxymethyl)benzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 334, or a pharmaceutically acceptable salt, solvate, hydrate, tautomer, or stereoisomer thereof.

In some embodiments, for Formula (I), A is bicyclic heteroaryl (e.g., 5-methoxy-2-(oxetan-3-yl)benzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 335, or a pharmaceutically acceptable salt, solvate, hydrate, tautomer, or stereoisomer thereof.

In some embodiments, for Formula (I), A is bicyclic heteroaryl (e.g., 5-hydroxy-2-(hydroxymethyl)benzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 336, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methyl)amino)pyrrolidinyl); L¹ and L² are absent; X, Y, and Z are N; and m and n are 0. In some embodiments, the compound of Formula (I) is Compound 337, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazyl); L¹ and L² are absent; X, Y, and Z are N; and m and n are 0. In some embodiments, the compound of Formula (I) is Compound 338, or a pharmaceutically acceptable salt, solvate, hydrate, tautomer, or stereoisomer thereof.

In some embodiments, for Formula (I), A is bicyclic heteroaryl (e.g., 5-hydroxy-1,2-dimethyl-1H-benzo[d]imidazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 339, 340, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(pyrrolidin-3-ylamino)-1-hydroxy-bicyclo[1.1.1]pentanyl); L¹ and L² are absent; X is C(R^{5a}) (e.g., —CH—); Y and Z are N; and m and n are 0. In some embodiments, the compound of Formula (I) is Compound 341, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 1,6-diazaspiro[3.4]octanyl); L¹ and L² are absent; X, Y, and Z are N; and m and n are 0. In some embodiments, the compound of Formula (I) is Compound 342, or a pharmaceutically acceptable salt, solvate, hydrate, tautomer, or stereoisomer thereof.

In some embodiments, for Formula (I), A is bicyclic heteroaryl (e.g., 4,6-dimethylpyrazolo[1,5-a]pyrazinyl); B is monocyclic heterocyclyl (e.g., piperdinyl); L¹ and L² are absent; X is C(R^{5a}) (e.g., —C(OCH₃)—); Y and Z are N; and m and n are 0. In some embodiments, the compound of Formula (I) is Compound 343, or a pharmaceutically acceptable salt, solvate, hydrate, tautomer, or stereoisomer thereof.

In some embodiments, for Formula (I), A is bicyclic heteroaryl (e.g., 5,7-dimethylimidazo[1,2-c]pyrimidinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 344, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazyl); L¹ and L² are absent; Y is C(R^{5a}) (e.g., —CH—); X and Z are N; and m and n are 0. In some embodiments, the compound of Formula (I) is Compound 345, or a pharmaceutically acceptable salt, solvate, hydrate, tautomer, or stereoisomer thereof.

In some embodiments, for Formula (I), A is bicyclic heteroaryl (e.g., 5-hydroxy-2-(ethyl)benzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 346, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 347, 349, 613, 614, 615, 616, 617, 618, 619, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 348, 350, 395, 396, 620, 621, 622, 623, 624, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 351, 352, 376, 377, 625, 626, 627, 628, 629, 630, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(1-(fluoromethyl)cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 353, 357, 553, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(1-(fluoromethyl)cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 354, 355, 554, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2-methylpiperidinyl); L¹ and L² are absent; X is C(R^{5a}) (e.g., —CH—); Y and Z are N; and m and n are 0. In some embodiments, the compound of Formula (I) is Compound 356, 555, 556, 557, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 358, 359, 631, 632, 633, 634, 635, 636, 637, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(1-(fluoromethyl)cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 360, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazyl); L¹ and L² are absent; X is C(R^{5a}) (e.g., —CH—); Y and Z are N; R³ is (e.g., —CH(OH)CH₃); m is 0; and n is 1. In some embodiments, the compound of Formula (I) is Compound 361, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazyl); L¹ and L² are absent; X is C(R^{5a}) (e.g., —CH—); Y and Z are N; R² is (e.g., —CH(OH)CH₃); m is 1; and n is 0. In some embodiments, the compound of Formula (I) is Compound 364, or a pharmaceutically acceptable salt, solvate, hydrate, tautomer, or stereoisomer thereof.

In some embodiments, for Formula (I), A is bicyclic heterocyclyl (e.g., 1-methyl-4,5,6,7-tetrahydro-1H-pyrazolo[4,3-c]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 365, 367, 559 or a pharmaceutically acceptable salt, solvate, hydrate, tautomer, or stereoisomer thereof.

In some embodiments, for Formula (I), A is bicyclic heterocyclyl (e.g., 2-methyl-4,5,6,7-tetrahydro-2H-pyrazolo[4,3-c]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 366, 490, 558, or a pharmaceutically acceptable salt, solvate, hydrate, tautomer, or stereoisomer thereof.

In some embodiments, for Formula (I), A is bicyclic heterocyclyl (e.g., 6-hydroxy-2,7-dimethyl-2H-indazolyl); B is monocyclic heterocyclyl (e.g., 3-(N-(methyl)amino)pyrrolidinyl); L¹ and L² are absent; X, Y, and Z are N; and m and n are 0. In some embodiments, the compound of Formula (I) is Compound 368, 369, or a pharmaceutically acceptable salt, solvate, hydrate, tautomer, or stereoisomer thereof.

In some embodiments, for Formula (I), A is bicyclic heterocyclyl (e.g., 6-hydroxy-2,7-dimethyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 370, or a pharmaceutically acceptable salt, solvate, hydrate, tautomer, or stereoisomer thereof.

In some embodiments, for Formula (I), A is bicyclic heterocyclyl (e.g., 8-cyano-2-methylimidazo[1,2-a]pyridinyl); B is monocyclic heterocyclyl (e.g., piperidinyl); L¹ and L² are absent; X, Y, and Z are N; and m and n are 0. In some embodiments, the compound of Formula (I) is Compound 371, or a pharmaceutically acceptable salt, solvate, hydrate, tautomer, or stereoisomer thereof.

In some embodiments, for Formula (I), A is bicyclic heteroaryl (e.g., 5-hydroxy-4-fluorobenzo[d]oxazolyl); B is monocyclic heterocyclyl (e.g., 3-(N-(1-(fluoromethyl)cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 372, 560, 561, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(1-(fluoromethyl)-cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 373, 491, 562, or a pharmaceutically acceptable salt, solvate, hydrate, tautomer, or stereoisomer thereof.

In some embodiments, for Formula (I), A is bicyclic heteroaryl (e.g., 5-hydroxy-4-fluoro-2-methylbenzo[d]oxazolyl); B is monocyclic heterocyclyl (e.g., 3-(N-(3-fluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 374, 375, 487, 638, 639, 640, 641, 642, 643, 644, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(1-methylcyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 378, 379, 563, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-aminopyrrolidinyl); L¹ and L² are absent; X, Y, and Z are N; and m and n are 0. In some embodiments, the compound of Formula (I) is Compound 380, 381, 564, or a pharmaceutically acceptable salt, solvate, hydrate, tautomer, or stereoisomer thereof.

In some embodiments, for Formula (I), A is bicyclic heterocyclyl (e.g., 8-cyano-2-methylimidazo[1,2-a]pyridinyl); B is monocyclic heterocyclyl (e.g., 3-(N-(methyl)amino)pyrrolidinyl); L¹ and L² are absent; X, Y, and Z are N; and m and n are 0. In some embodiments, the compound of Formula (I) is Compound 382, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., 2-methylpiperidinyl); L¹ and L² are absent; X is C(R^{5a}) (e.g., —CH—); Y and Z are N; and m and n are 0. In some embodiments, the compound of Formula (I) is Compound 383, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(3,3-difluorocyclobutyl)amino)pyrrolidinyl); L¹ and L² are absent; X is C(R^{5a}) (e.g., —CH—); Y and Z are N; and m and n are 0. In some embodiments, the compound of Formula (I) is Compound 384, 385, 565, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-hydroxypiperidinyl); L¹ and L² are absent; X is C(R^{5a}) (e.g., —CH—); Y and Z are N; and m and n are 0. In some embodiments, the compound of Formula (I) is Compound 386, 566, 567, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., 3-(N-(cyclopropylmethyl)amino)pyrrolidinyl); L¹ and L² are absent; X is C(R^{5a}) (e.g., —CH—); Y and Z are N; and m and n are 0. In some embodiments, the compound of Formula (I) is Compound 387, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is Compound 388, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(cyclopropylmethyl)amino)pyrrolidinyl); L¹ and L² are absent; X is C(R^{5a}) (e.g., —CH—); Y and Z are N; and m and n are 0. In some embodiments, the compound of Formula (I) is Compound 389, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(cyclopropylmethyl)amino)pyrrolidinyl); L¹ and L² are absent; X is C(R^{5a}) (e.g., —CH—); Y and Z are N; and m and n are 0. In some embodiments, the compound of Formula (I) is Compound 390, or a pharmaceutically acceptable salt, solvate, hydrate, tautomer, or stereoisomer thereof.

In some embodiments, for Formula (I), A is bicyclic heteroaryl (e.g., 7-fluoro-6-hydroxy-2-methyl-2H-indazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 391, 392, or a pharmaceutically acceptable salt, solvate, hydrate, tautomer, or stereoisomer thereof.

In some embodiments, for Formula (I), A is bicyclic heterocyclyl (e.g., 6-hydroxy-2,7-dimethyl-2H-indazolyl); B is monocyclic heterocyclyl (e.g., pyrrolidinyl); L¹ and L² are absent; X, Y, and Z are N; and m and n are 0. In some embodiments, the compound of Formula (I) is Compound 397, 398, 399, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., piperidinyl); L¹ and L² are absent; X is C(R^{5a}) (e.g., —CH—); Y is C(R^{5a}) (e.g., —C(OH)—); and Z are N; and m and n are 0. In some embodiments, the compound of Formula (I) is Compound 400, or a pharmaceutically acceptable salt, solvate, hydrate, tautomer, or stereoisomer thereof.

In some embodiments, for Formula (I), A is bicyclic heteroaryl (e.g., 5-hydroxy-2,4-dimethylbenzo[d]oxazolyl); B is monocyclic heterocyclyl (e.g., 3-(N-(isopropyl)amino)pyrrolidinyl); L¹ and L² are absent; X is C(R^{5a}) (e.g., —CH—); Y and Z are N; and m and n are 0. In some embodiments, the compound of Formula (I) is 401, 402, or a pharmaceutically acceptable salt, solvate, hydrate, tautomer, or stereoisomer thereof.

In some embodiments, for Formula (I), A is bicyclic heterocyclyl (e.g., 7-cyano-6-hydroxy-2-methyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 403, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); A is bicyclic heteroaryl (e.g., 6-hydroxy-2-methyl-2H-indazolyl); L¹ and L² are absent; X, Y, and Z are N; and m and n are 0. In some embodiments, the compound of Formula (I) is Compound 404, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); A is monocyclic heteroaryl (e.g., 3-methoxypyridazinyl); L¹ and L² are absent; X, Y, and Z are N; and m and n are 0. In some embodiments, the compound of Formula (I) is Compound 405, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridinyl); B is monocyclic heterocyclyl (e.g., 3-(N-(methyl)amino)pyrrolidinyl); L¹ and L² are absent; X, Y and Z are N; and m and n are 0. In some embodiments, the compound of Formula (I) is Compound 406, 407, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., piperazinyl); L¹ and L² are absent; X is C(R^{5a}) (e.g., —CH—); Y and Z are N; and m and n are 0. In some embodiments, the compound of Formula (I) is Compound 408, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2-methylpiperazinyl); L¹ and L² are absent; X is C(R^{5a}) (e.g., —CH—); Y and Z are N; and m and n are 0. In some embodiments, the compound of Formula (I) is Compound 409, 568, 569, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is bicyclic heterocyclyl (e.g., octahydropyrrolo[1,2-a]pyrazinyl); L¹ and L² are absent; X is C(R^{5a}) (e.g., —CH—); Y and Z are N; and m and n are 0. In some embodiments, the compound of Formula (I) is Compound 410, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is bicyclic heterocyclyl (e.g., 4,7-diazaspiro[2.5]octanyl); L¹ and L² are absent; X is C(R^{5a}) (e.g., —CH—); Y and Z are N; and m and n are 0. In some embodiments, the compound of Formula (I) is Compound 411, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is bicyclic heterocyclyl (e.g., 4-(N-(tert-butyl)amino)piperidinyl); L¹ and L² are absent; X is C(R^{5a}) (e.g., —CH—); Y and Z are N; and m and n are 0. In some embodiments, the compound of Formula (I) is Compound 412, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); A is bicyclic heteroaryl (e.g., 5-hydroxy-2-methylbenzo[d]oxazolyl); L¹ and L² are absent; X, Y, and Z are N; and m and n are 0. In some embodiments, the compound of Formula (I) is Compound 413, 414, 570, or a pharmaceutically acceptable salt, solvate, hydrate, tautomer, or stereoisomer thereof.

In some embodiments, for Formula (I), A is monocyclic heterocyclyl (e.g., 3-(N-(isopropyl)amino)pyrrolidinyl); A is bicyclic heteroaryl (e.g., 5-hydroxy-2,4-dimethylbenzo[d]oxazolyl); L¹ and L² are absent; X, Y, and Z are N; and m and n are 0. In some embodiments, the compound of Formula (I) is Compound 415, 416, 571, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); B is bicyclic heteroaryl (e.g., 5-hydroxy-2-methylbenzo[d]oxazolyl); L¹ and L² are absent; X and Z are N; Y is C(R^{5a}) (e.g., —CH—); and m and n are 0. In some embodiments, the compound of Formula (I) is Compound 417, 572, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2-cyclopropylpiperazinyl); L¹ and L² are absent; X is C(R^{5a}) (e.g., —CH—); Y and Z are N; and m and n are 0. In some embodiments, the compound of Formula (I) is Compound 418, 421, 573, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2-cyclopropyl-6-methylpiperazinyl); L¹ and L² are absent; X is C(R^{5a}) (e.g., —CH—); Y and Z are N; and m and n are 0. In some embodiments, the compound of Formula (I) is Compound 419, 574, 575, 576, 577, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2,6-dimethylpiperazinyl); L¹ and L² are absent; X is C(R^{5a}) (e.g., —CH—); Y and Z are N; and m and n are 0. In some embodiments, the compound of Formula (I) is Compound 420, 578, 579, 580, 581, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2-isopropylpiperazinyl); L¹ and L² are absent; X is C(R^{5a}) (e.g., —CH—); Y and Z are N; and m and n are 0. In some embodiments, the compound of Formula (I) is Compound 422, 582, 583, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 2-tert-butylpiperazinyl); L¹ and L² are absent; X is C(R^{5a}) (e.g., —CH—); Y and Z are N; and m and n are 0. In some embodiments, the compound of Formula (I) is Compound 423, 492, 584, 585, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is bicyclic heterocyclyl (e.g., 4-(N-amino)piperidinyl); L¹ and L² are absent; X is C(R^{5a}) (e.g., —CH—); Y and Z are N; and m and n are 0. In some embodiments, the compound of Formula (I) is Compound 424, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is bicyclic heterocyclyl (e.g., 4-(N-methylamino)piperidinyl); L¹ and L² are absent; X is C(R^{5a}) (e.g., —CH—); Y and Z are N; and m and n are 0. In some embodiments, the compound of Formula (I) is Compound 425, or a pharmaceutically acceptable salt, solvate, hydrate, tautomer, or stereoisomer thereof.

In some embodiments, for Formula (I), A is bicyclic heteroaryl (e.g., 2-methyloxazolo[5,4-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 426, 427, 586, or a pharmaceutically acceptable salt, solvate, hydrate, tautomer, or stereoisomer thereof.

In some embodiments, for Formula (I), A is bicyclic heteroaryl (e.g., 2,7-dimethyloxazolo[5,4-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 428, 429, 587, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyloxazolo[5,4-b]pyridinyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 430, 431, 588, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); A is bicyclic heteroaryl (e.g., 5-hydroxy-2,4-dimethylbenzo[d]oxazolyl); L¹ and L² are absent; X, Y, and Z are N; and m and n are 0. In some embodiments, the compound of Formula (I) is Compound 432, 433, 589, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); A is bicyclic heteroaryl (e.g., 5-hydroxy-2-methylbenzo[d]oxazolyl); L¹ and L² are absent; X, Y, and Z are N; and m and n are 0. In some embodiments, the compound of Formula (I) is Compound 434, 435, 590, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); A is bicyclic heteroaryl (e.g., 5-hydroxy-2,4-dimethylbenzo[d]oxazolyl); L¹ and L² are absent; X, Y, and Z are N; and m and n are 0. In some embodiments, the compound of Formula (I) is Compound 436, 437, 591, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); A is bicyclic heteroaryl (e.g., 6-hydroxy-2,7-dimethyl-2H-indazolyl; L¹ and L² are absent; X, Y, and Z are N; and m and n are 0. In some embodiments, the compound of Formula (I) is Compound 438, 439, 592, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); B is bicyclic heteroaryl (e.g., 5-hydroxy-2,4-dimethylbenzo[d]oxazolyl); L¹ and L² are absent; X and Z are N; Y is C(R^{5a}) (e.g., —CH—); and m and n are 0. In some embodiments, the compound of Formula (I) is Compound 440, 441, 593, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); B is bicyclic heteroaryl (e.g., 5-hydroxy-2-methylbenzo[d]oxazolyl); L¹ and L² are absent; X and Z are N; Y is C(R^{5a}) (e.g., —CH—); and m and n are 0. In some embodiments, the compound of Formula (I) is Compound 442, 443, 594, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); B is bicyclic heteroaryl (e.g., 5-hydroxy-2,4-dimethylbenzo[d]oxazolyl); L¹ and L² are absent; X and Z are N; Y is C(R^{5a}) (e.g., —CH—); and m and n are 0. In some embodiments, the compound of Formula (I) is Compound 444, 445, 595, or a pharmaceutically acceptable salt, solvate, hydrate, tautomer, or stereoisomer thereof.

In some embodiments, for Formula (I), A is monocyclic heterocyclyl (e.g., 3-(N-(cyclopropyl)amino)pyrrolidinyl); B is bicyclic heteroaryl (e.g., 6-hydroxy-2,7-dimethyl-2H-indazolyl); L¹ and L² are absent; X and Z are N; Y is C(R^{5a}) (e.g., —CH—); and m and n are 0. In some embodiments, the compound of Formula (I) is Compound 446, 447, 596 or a pharmaceutically acceptable salt, solvate, hydrate, tautomer, or stereoisomer thereof.

In some embodiments, for Formula (I), A is bicyclic heterocyclyl (e.g., 4-azaspiro[2.5]octanyl); A is bicyclic heteroaryl (e.g., 6-hydroxy-2-methyl-2H-indazolyl); L¹ and L² are absent; X, Y, and Z are N; and m and n are 0. In some embodiments, the compound of Formula (I) is Compound 448, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); B is bicyclic heteroaryl (e.g., 6-hydroxy-2-methyl-2H-indazolyl); L¹ and L² are absent; X and Z are N; Y is C(R^{5a}) (e.g., —CH—); and m and n are 0. In some embodiments, the compound of Formula (I) is Compound 449, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); B is bicyclic heteroaryl (e.g., 6-hydroxy-2-methyl-2H-indazolyl); L¹ and L² are absent; X and Z are N; Y is C(R^{5a}) (e.g., —CH—); and m and n are 0. In some embodiments, the compound of Formula (I) is Compound 450, or a pharmaceutically acceptable salt, solvate, hydrate, tautomer, or stereoisomer thereof.

In some embodiments, for Formula (I), A is monocyclic heterocyclyl (e.g., 3-(N-(methylcyclopropyl)amino)pyrrolidinyl); B is bicyclic heteroaryl (e.g., 6-hydroxy-2-methyl-2H-indazolyl); L¹ and L² are absent; X is C(R^{5a}) (e.g., —CH—); Y and Z are N; and m and n are 0. In some embodiments, the compound of Formula (I) is Compound 451, 452, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-a]pyrazinyl); B is monocyclic heterocyclyl (e.g., piperazinyl); L¹ and L² are absent; X and Z are N; Y is C(R^{5a}) (e.g., —CH—); and m and n are 0. In some embodiments, the compound of Formula (I) is Compound 453, or a pharmaceutically acceptable salt, solvate, hydrate, tautomer, or stereoisomer thereof.

In some embodiments, for Formula (I), A is bicyclic heteroaryl (e.g., 7-cyano-6-hydroxy-2-methyl-2H-indazolyl); B is monocyclic heterocyclyl (e.g., piperidinyl); L¹ and L² are absent; X, Y, and Z are N; and m and n are 0. In some embodiments, the compound of Formula (I) is Compound 454, or a pharmaceutically acceptable salt, solvate, hydrate, tautomer, or stereoisomer thereof.

In some embodiments, for Formula (I), A is monocyclic heterocyclyl (e.g., 3-(N-(tert-butyl)amino)pyrrolidinyl); B is bicyclic heteroaryl (e.g., 6-hydroxy-2-methyl-2H-indazolyl); L¹ and L² are absent; X and Z are N; Y is C(R^{5a}) (e.g., —CH—); and m and n are 0. In some embodiments, the compound of Formula (I) is Compound 455, or a pharmaceutically acceptable salt, solvate, hydrate, tautomer, or stereoisomer thereof.

In some embodiments, for Formula (I), A is bicyclic heterocyclyl (e.g., 4-azaspiro[2.5]octanyl); A is bicyclic heteroaryl (e.g., 6-hydroxy-2-methyl-2H-indazolyl); L¹ and L² are absent; X is C(R^{5a}) (e.g., —CH—); Y and Z are N; and m and n are 0. In some embodiments, the compound of Formula (I) is Compound 456, 495, 496, or a pharmaceutically acceptable salt, solvate, hydrate, tautomer, or stereoisomer thereof.

In some embodiments, for Formula (I), A is monocyclic heteroaryl (e.g., pyrazolyl); B is monocyclic heterocyclyl (e.g., piperidinyl); L¹ is absent; L² is —C(O)N(R⁴)— (e.g., —C(O)N(H)—); X and Y are each independently C(R^{5a}) (e.g., —CH—); Z is N; and m and n are 0. In some embodiments, the compound of Formula (I) is Compound 457, or a pharmaceutically acceptable salt, solvate, hydrate, tautomer, or stereoisomer thereof.

In some embodiments, for Formula (I), A is bicyclic heterocyclyl (e.g., 4-azaspiro[2.5]oct-6-enyl); B is bicyclic heteroaryl (e.g., 6-hydroxy-2-methyl-2H-indazolyl); L¹ and L² are absent; X, Y, and Z are N; and m and n are 0. In some embodiments, the compound of Formula (I) is Compound 458, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methyl)amino)-4-methylpyrrolidinyl); L¹ and L² are absent; X is C(R^{5a}) (e.g., —CH—); Y and Z are N; and m and n are 0. In some embodiments, the compound of Formula (I) is Compound 459, 597, 598, 599, 600, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methyl)amino)-4-fluoropyrrolidinyl); L¹ and L² are absent; X is C(R^{5a}) (e.g., —CH—); Y and Z are N; and m and n are 0. In some embodiments, the compound of Formula (I) is Compound 460, 461, 462, 463, 601, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(tert-butyl)amino)-4-methylpyrrolidinyl); L¹ and L² are absent; X is C(R^{5a}) (e.g., —CH—); Y and Z are N; and m and n are 0. In some embodiments, the compound of Formula (I) is Compound 464, 465, 466, 467, 602, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 3-(N-(tert-butyl)amino)-4-fluoropyrrolidinyl); L¹ and L² are absent; X is C(R^{5a}) (e.g., —CH—); Y and Z are N; and m and n are 0. In some embodiments, the compound of Formula (I) is Compound 468, 469, 470, 471, 603, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(tert-butyl)amino)-4-fluoropyrrolidinyl); L¹ and L² are absent; X is C(R^{5a}) (e.g., —CH—); Y and Z are N; and m and n are 0. In some embodiments, the compound of Formula (I) is Compound 472, 473, 474, 475, 604, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(tert-butyl)amino)-4-methylpyrrolidinyl); L¹ and L² are absent; X is C(R^{5a}) (e.g., —CH—); Y and Z are N; and m and n are 0. In some embodiments, the compound of Formula (I) is Compound 476, 477, 478, 479, 605, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-(methyl)amino)-4-methylpyrrolidinyl); L¹ and L² are absent; X is C(R^{5a}) (e.g., —CH—); Y and Z are N; and m and n are 0. In some embodiments, the compound of Formula (I) is Compound 480, 481, 606, 607, 608, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X is N; Y and Z are each independently is C(R^{5a}) (e.g., —CH—); R² is halo (e.g., chloro); R³ is C₁-C₆ alkyl (e.g., methyl); m is 1; and n is 1. In some embodiments, the compound of Formula (I) is Compound 482, 484, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X is N; Y and Z are each independently is C(R^{5a}) (e.g., —CH—); R² is —C(O)NR^{B}R^{C} (e.g., —C(O)NHCH₃); R³ is halo (e.g., fluoro); m is 1; and n is 1. In some embodiments, the compound of Formula (I) is Compound 483, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 4-(N,N-dimethyl)aminopiperidinyl); L¹ and L² are absent; X is N; Y and Z are each independently is C(R^{5a}) (e.g., —CH—); R² is halo (e.g., chloro); R³ is C₁-C₆ alkyl (e.g., methyl); m is 1; and n is 1. In some embodiments, the compound of Formula (I) is Compound 485, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X is N; Y and Z are each independently is C(R^{5a}) (e.g., —CH—); R² is halo (e.g., chloro); R³ is C₁-C₆ alkyl (e.g., methyl); m is 1; and n is 1. In some embodiments, the compound of Formula (I) is Compound 486, 748, 749, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(1-(fluoromethyl)cyclopropyl)amino)pyrrolidinyl); L¹ and L² are absent; X is C(R^{5a}) (e.g., —CH—); Y and Z are N; and m and n are 0. In some embodiments, the compound of Formula (I) is Compound 488, 489, 645, or a pharmaceutically acceptable salt, solvate, hydrate, tautomer, or stereoisomer thereof.

In some embodiments, for Formula (I), A is bicyclic heteroaryl (e.g., 5-hydroxy-2-methylbenzo[d]oxazolyl); B is monocyclic heterocyclyl (e.g., 3-(N-(tert-butyl)-N-methylamino)pyrrolidinyl); L¹ and L² are absent; X is C(R^{5a}) (e.g., —CH—); Y and Z are N; and m and n are 0. In some embodiments, the compound of Formula (I) is Compound 493, 494, 646, or a pharmaceutically acceptable salt, solvate, hydrate, tautomer, or stereoisomer thereof.

In some embodiments, for Formula (I), A is bicyclic heterocyclyl (e.g., 4-azaspiro[2.5]octanyl); A is bicyclic heteroaryl (e.g., 5-hydroxy-2-methylbenzo[d]oxazolyl); L¹ and L² are absent; X is C(R^{5a}) (e.g., —CH—); Y and Z are N; and m and n are 0. In some embodiments, the compound of Formula (I) is Compound 497, 498, 647, or a pharmaceutically acceptable salt, solvate, hydrate, tautomer, or stereoisomer thereof.

In some embodiments, for Formula (I), A is bicyclic heterocyclyl (e.g., 4-azaspiro[2.5]octanyl); A is bicyclic heteroaryl (e.g., 5-hydroxy-2,6-dimethylbenzo[d]oxazolyl); L¹ and L² are absent; X is C(R^{5a}) (e.g., —CH—); Y and Z are N; and m and n are 0. In some embodiments, the compound of Formula (I) is Compound 499, 500, 648, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 501, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)N(CH₃)₂); m is 1; and n is 0. In some embodiments, the compound of Formula (I) is Compound 502, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)NHCH₂CH₃); m is 1; and n is 0. In some embodiments, the compound of Formula (I) is Compound 503, or a pharmaceutically acceptable salt, solvate, hydrate, tautomer, or stereoisomer thereof.

In some embodiments, for Formula (I), A is bicyclic heteroaryl (e.g., 7-hydroxy-2,8-dimethylimidazo[1,2-a]pyridinyl); B is monocyclic heterocyclyl (e.g., 3-(N-(methyl)amino)pyrrolidinyl); L¹ and L² are absent; X, Y, and Z are N; and m and n are 0. In some embodiments, the compound of Formula (I) is Compound 504, 649, 650, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 505, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 506, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 4-(N,N-dimethylamino)piperidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 507, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —NR^{B}R^{C} (e.g., —NHCH₃); m is 1; and n is 0. In some embodiments, the compound of Formula (I) is Compound 508, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —NR^{B}R^{C} (e.g., —N(CH₃)₂); m is 1; and n is 0. In some embodiments, the compound of Formula (I) is Compound 509, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-methylamino)pyrrolidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 510, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 511, 651, 652, 653, 654, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2-methylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 512, 655, 656. or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., piperazinyl); L¹ and L² are absent; X and Y are N; Z is C(R^{5a}) (e.g., —CH—); R² is —C(O)R^{D} (e.g., —C(O)CH₃); m is 1; and n is 0. In some embodiments, the compound of Formula (I) is Compound 513, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., piperazinyl); L¹ and L² are absent; X and Y are N; Z is C(R^{5a}) (e.g., —CH—); R² is cyano; m is 1; and n is 0. In some embodiments, the compound of Formula (I) is Compound 514, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 515, or a pharmaceutically acceptable salt, solvate, hydrate, tautomer, or stereoisomer thereof.

In some embodiments, for Formula (I), A is bicyclic heteroaryl (e.g., 7-fluoro-2H-indazolyl); B is monocyclic heterocyclyl (e.g., piperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 516, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., piperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 517, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., piperazinyl); L¹ and L² are absent; X and Y are N; Z is C(R^{5a}) (e.g., —CH—); R² is cycloalkyl (e.g., cyclopropyl); m is 1; and n is 0.

In some embodiments, the compound of Formula (I) is Compound 518, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is heteroaryl (e.g., thiazolyl); m is 1; and n is 0. In some embodiments, the compound of Formula (I) is Compound 519, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); m is 0; and n is 0. In some embodiments, the compound of Formula (I) is Compound 520, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X and Y are N; Z is C(R^{5a}) (e.g., —CH—); m is 0; and n is 0. In some embodiments, the compound of Formula (I) is Compound 521, 657, 658, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is heteroaryl (e.g., triazolyl); m is 1; and n is 0. In some embodiments, the compound of Formula (I) is Compound 522, 538, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —C(O)NR^{B}R^{C} (e.g., —C(O)NH₂); m is 1; and n is 0. In some embodiments, the compound of Formula (I) is Compound 523, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —C(O)OR^{D} (e.g., —C(O)OH); m is 1; and n is 0. In some embodiments, the compound of Formula (I) is Compound 524, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 525, 659, 660, 661, 662, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X, Y, and Z are N; m is 0; and n is 0. In some embodiments, the compound of Formula (I) is Compound 526, 663, 664, 665, 666, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 527, 667, 668, 669, 670, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 4-(N,N-dimethyl)aminopiperidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 528, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2-methylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 529, 671, 672, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2-methylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 530, 673, 674, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 531, 675, 676, 677, 678, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 4,7-diazaspiro[2.5]octanyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 532, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X, Y, and Z are N; R² is —C(O)NR^{B}R^{C} (e.g., —C(O)NHCH₃); m is 1; and n is 0. In some embodiments, the compound of Formula (I) is Compound 533, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X, Y, and Z are N; R² is —C(O)NR^{B}R^{C} (e.g., —C(O)NHCH₃); m is 1; and n is 0. In some embodiments, the compound of Formula (I) is Compound 534, 679, 680, 681, 682, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH₂CH₃); m is 1; and n is 0. In some embodiments, the compound of Formula (I) is Compound 535, 683, 684, 685, 686, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 3-(N-methyl)aminopyrrolidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 536, 537, 687, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH(CH₃)₂); m is 1; and n is 0. In some embodiments, the compound of Formula (I) is Compound 539, 688, 689, 690, 691, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NH(cyclobutyl)); m is 1; and n is 0. In some embodiments, the compound of Formula (I) is Compound 540, 692, 693, 694, 695, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is heteroaryl (e.g., tetrazolyl); m is 1; and n is 0. In some embodiments, the compound of Formula (I) is Compound 541, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is cycloalkyl (e.g., cyclopropyl); m is 1; and n is 0. In some embodiments, the compound of Formula (I) is Compound 542, 696, 697, 698, 699, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-methyl)aminopyrrolidinyl); L¹ and L² are absent; X and Y are N; Z is C(R^{5a}) (e.g., —CH—); R² is cycloalkyl (e.g., cyclopropyl); m is 1; and n is 0. In some embodiments, the compound of Formula (I) is Compound 543, 700, 701, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., 2,6-dimethylpiperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 544, 702, 703, 704, 705, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 706, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is cyano; m is 1; and n is 0. In some embodiments, the compound of Formula (I) is Compound 707, 708, 709, 710, 711, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NH₂); m is 1; and n is 0. In some embodiments, the compound of Formula (I) is Compound 712, 713, 714, 715, 716, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)R^{D} (e.g., —C(O)CH₃); m is 1; and n is 0. In some embodiments, the compound of Formula (I) is Compound 717, 718, 719, 720, 721, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 722, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X is N; Y and Z are each independently is C(R^{5a}) (e.g., —CH—); R² is halo (e.g., chloro); R³ is C₁-C₆ alkyl (e.g., methyl); m is 1; and n is 1. In some embodiments, the compound of Formula (I) is Compound 723, 724, 725, 726, 727, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is bicyclic heterocyclyl (e.g., 4,7-diazaspiro[2.5]octanyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 728, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-methyl)aminopyrrolidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 729, 730, 731, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is heteroaryl (e.g., thiazolyl); m is 1; and n is 0. In some embodiments, the compound of Formula (I) is Compound 732, 733 734, 735, 736, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NHCH₂CH₂OH); m is 1; and n is 0. In some embodiments, the compound of Formula (I) is Compound 737, 738, 739, 740, 741, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 4-(N,N-dimethyl)aminopiperidinyl); L¹ and L² are absent; X and Y are N; Z is C(R^{5a}) (e.g., —CH—); R² is —C(O)NR^{B}R^{C} (e.g., —C(O)NHCH₃); m is 1; and n is 0. In some embodiments, the compound of Formula (I) is Compound 742, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 743, 744, 745, 746, 747, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); m is 0; and n is 0. In some embodiments, the compound of Formula (I) is Compound 750, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 751, 752, 753, 754, 755, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 4-(N,N-diethyl)aminopiperidinyl); L¹ and L² are absent; X and Y are N; Z is C(R^{5a}) (e.g., —CH—); m is 0; and n is 0. In some embodiments, the compound of Formula (I) is Compound 756, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 4-(N,N-dimethyl)aminopiperidinyl); L¹ and L² are absent; X and Y are N; Z is C(R^{5a}) (e.g., —CH—); m is 0; and n is 0. In some embodiments, the compound of Formula (I) is Compound 757, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2,6-dimethylpiperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 758, 759, 760, 761, 762, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 2,6-dimethylpiperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 763, 764, 765, 766, 767, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X is N; Y and Z are each independently is C(R^{5a}) (e.g., —CH—); R³ is halo (e.g., fluoro); m is 0; and n is 1. In some embodiments, the compound of Formula (I) is Compound 768, 769, 770, 771, 772, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl; B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 773, 774, 775, 776, 777, 803, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., piperazinyl); L¹ and L² are absent; X and Y are N; Z is C(R^{5a}) (e.g., —CH—); R² is —OR^{A} (e.g., —OCH₃); m is 1; and n is 0. In some embodiments, the compound of Formula (I) is Compound 778, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NH(cyclobutyl)); m is 1; and n is 0. In some embodiments, the compound of Formula (I) is Compound 779, 839, 840, 841, 842, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 780, 802, 843, 844, 845, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is C₁-C₆-alkyl (e.g., methyl); m is 1; and n is 0. In some embodiments, the compound of Formula (I) is Compound 781, 849, 850, 851, 852, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is C₁-C₆-alkyl (e.g., methyl); m is 1; and n is 0. In some embodiments, the compound of Formula (I) is Compound 782, 806, 846, 847, 848, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is C₁-C₆-alkyl (e.g., methyl); m is 1; and n is 0. In some embodiments, the compound of Formula (I) is Compound 783, 807, 853, 854, 855, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NH₂); m is 1; and n is 0. In some embodiments, the compound of Formula (I) is Compound 784, 796, 856, 857, 858, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); R² is —C(O)NR^{B}R^{C} (e.g., —C(O)NH₂); m is 1; and n is 0. In some embodiments, the compound of Formula (I) is Compound 785, 797, 859, 860, 861, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); m is 0; and n is 0. In some embodiments, the compound of Formula (I) is Compound 786, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); m is 0; and n is 0. In some embodiments, the compound of Formula (I) is Compound 787, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); R² is C₁-C₆-alkyl (e.g., methyl); m is 1; and n is 0. In some embodiments, the compound of Formula (I) is Compound 788, or a pharmaceutically acceptable salt, solvate, hydrate, tautomer, or stereoisomer thereof.

In some embodiments, for Formula (I), A is bicyclic heteroaryl (e.g., 7-fluoro-2-methyl-2H-indazolyl); B is monocyclic heterocyclyl (e.g., 2-methylpiperazinyl); L¹ and L² are absent; Y is N; X and Z are C(R^{5a}) (e.g., —CH—); R³ is halo (e.g., fluoro); m is 1; and n is 0. In some embodiments, the compound of Formula (I) is Compound 789, 862, 863, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazyl); L¹ and L² are absent; X and Y are N; Z is C(R^{5a}) (e.g., —CH—); R² is C₁-C₆-alkyl (e.g., cyclobutyl); m is 1; and n is 0. In some embodiments, the compound of Formula (I) is Compound 790, 794, 864, 865, 866, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 2,6-dimethylpiperazyl); L¹ and L² are absent; X and Y are N; Z is C(R^{5a}) (e.g., —CH—); R² is C₁-C₆-alkyl (e.g., cyclobutyl); m is 1; and n is 0. In some embodiments, the compound of Formula (I) is Compound 791, 867, 868, 869, 870, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 4,7-diazaspiro[2.5]octanyl); L¹ and L² are absent; X and Y are N; Z is C(R^{5a}) (e.g., —CH—); m is 0; and n is 0. In some embodiments, the compound of Formula (I) is Compound 792, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 4,7-diazaspiro[2.5]octanyl); L¹ and L² are absent; X and Y are N; Z is C(R^{5a}) (e.g., —CH—); R² is C₁-C₆-alkyl (e.g., methyl); m is 1; and n is 0. In some embodiments, the compound of Formula (I) is Compound 793, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4,7-diazaspiro[2.5]octanyl); L¹ and L² are absent; X and Y are N; Z is C(R^{5a}) (e.g., —CH—); m is 0; and n is 0. In some embodiments, the compound of Formula (I) is Compound 795, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2,6-dimethylpiperazyl); L¹ and L² are absent; X and Y are N; Z is C(R^{5a}) (e.g., —CH—); m is 0; and n is 0. In some embodiments, the compound of Formula (I) is Compound 798, 871, 872, 873, 874, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is monocyclic heterocyclyl (e.g., 2,6-dimethylpiperazyl); L¹ and L² are absent; Y is N; X and Z are C(R^{5a}) (e.g., —CH—); R³ is halo (e.g., fluoro); m is 1; and n is 0. In some embodiments, the compound of Formula (I) is Compound 799, 875, 876, 877, 878, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is monocyclic heterocyclyl (e.g., 2,6-dimethylpiperazyl); L¹ and L² are absent; Y is N; X and Z are C(R^{5a}) (e.g., —CH—); R³ is halo (e.g., fluoro); m is 1; and n is 0. In some embodiments, the compound of Formula (I) is Compound 800, 879, 880, 881, 882, or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is monocyclic heterocyclyl (e.g., 4-(azetidin-1-yl)piperidinyl); L¹ and L² are absent; X and Y are N; Z is C(R^{5a}) (e.g., —CH—); m is 0; and n is 0. In some embodiments, the compound of Formula (I) is Compound 801, or a pharmaceutically acceptable salt, solvate, hydrate, tautomer, or stereoisomer thereof.

In some embodiments, for Formula (I), A is bicyclic heteroaryl (e.g., 2,8-dimethylimidazo[1,2-b]pyridazyl); B is bicyclic heterocyclyl (e.g., 4,7-diazaspiro[2.5]octanyl); L¹ and L² are absent; Y is N; X and Z are C(R^{5a}) (e.g., —CH—); R³ is halo (e.g., fluoro); m is 1; and n is 0. In some embodiments, the compound of Formula (I) is Compound 804, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 4,7-diazaspiro[2.5]octanyl); L¹ and L² are absent; Y is N; X and Z are C(R^{5a}) (e.g., —CH—); R³ is halo (e.g., fluoro); m is 1; and n is 0. In some embodiments, the compound of Formula (I) is Compound 805, or a pharmaceutically acceptable salt, solvate, hydrate, tautomer, or stereoisomer thereof.

In some embodiments, for Formula (I), A is bicyclic heteroaryl (e.g., 5-hydroxy-2-ethylbenzo[d]oxazolyl); B is monocyclic heterocyclyl (e.g., 3-(N-(tert-butyl)amino)pyrrolidinyl); L¹ and L² are absent; X is C(R^{5a}) (e.g., —CH—); Y and Z are N; and m and n are 0. In some embodiments, the compound of Formula (I) is Compound 808, 809, 883, or a pharmaceutically acceptable salt, solvate, hydrate, tautomer, or stereoisomer thereof.

In some embodiments, for Formula (I), A is bicyclic heteroaryl (e.g., 6-hydroxy-2,7-dimethyl-2H-indazolyl); B is bicyclic heterocyclyl (e.g., 5-methyl-4,7-diazaspiro[2.5]octanyl); L¹ and L² are absent; X and Y are N; Z is C(R^{5a}) (e.g., —CH—); m is 0; and n is 0. In some embodiments, the compound of Formula (I) is Compound 810, 811, 884 or a pharmaceutically acceptable salt, solvate, hydrate, tautomer, or stereoisomer thereof.

In some embodiments, for Formula (I), A is bicyclic heteroaryl (e.g., 6-hydroxy-2-methyl-2H-indazolyl); B is bicyclic heterocyclyl (e.g., 5-methyl-4,7-diazaspiro[2.5]octanyl); L¹ and L² are absent; X and Y are N; Z is C(R^{5a}) (e.g., —CH—); m is 0; and n is 0. In some embodiments, the compound of Formula (I) is Compound 812, 813, 885, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is bicyclic heterocyclyl (e.g., 5-methyl-4,7-diazaspiro[2.5]octanyl); L¹ and L² are absent; X and Y are N; Z is C(R^{5a}) (e.g., —CH—); m is 0; and n is 0. In some embodiments, the compound of Formula (I) is Compound 814, 815, 886, or a pharmaceutically acceptable salt, solvate, hydrate, tautomer, or stereoisomer thereof.

In some embodiments, for Formula (I), A is bicyclic heteroaryl (e.g., 8-methoxy-2-methylimidazo[1,2-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 816, 830, 887, 888, 889, or a pharmaceutically acceptable salt, solvate, hydrate, tautomer, or stereoisomer thereof.

In some embodiments, for Formula (I), A is bicyclic heteroaryl (e.g., 8-cyano-2-methylimidazo[1,2-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 817, 831, 890, 891, 892, or a pharmaceutically acceptable salt, solvate, hydrate, tautomer, or stereoisomer thereof.

In some embodiments, for Formula (I), A is bicyclic heteroaryl (e.g., 8-chloro-2-methylimidazo[1,2-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 818, 893, 894, 895, 896, or a pharmaceutically acceptable salt, solvate, hydrate, tautomer, or stereoisomer thereof.

In some embodiments, for Formula (I), A is bicyclic heteroaryl (e.g., 8-fluoro-2-methyl-[1,2,4]triazolo[1,5-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 819, 825, 897, 898, 899, or a pharmaceutically acceptable salt, solvate, hydrate, tautomer, or stereoisomer thereof.

In some embodiments, for Formula (I), A is bicyclic heteroaryl (e.g., 8-fluoro-[1,2,4]triazolo[4,3-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 820, 833, 900, 901, 902, or a pharmaceutically acceptable salt, solvate, hydrate, tautomer, or stereoisomer thereof.

In some embodiments, for Formula (I), A is bicyclic heteroaryl (e.g., 2-ethyl-8-fluoroimidazo[1,2-a]pyridinyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 821, 836, 903, 904, 905, or a pharmaceutically acceptable salt, solvate, hydrate, tautomer, or stereoisomer thereof.

In some embodiments, for Formula (I), A is bicyclic heteroaryl (e.g., 3,8-difluoro-2-methylimidazo[1,2-a]pyridinyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 822, 837, 906, 907, 908, or a pharmaceutically acceptable salt, solvate, hydrate, tautomer, or stereoisomer thereof.

In some embodiments, for Formula (I), A is bicyclic heteroaryl (e.g., 8-methoxy-2-methylimidazo[1,2-a]pyrazinyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 823, 838, 909, 910, 911, or a pharmaceutically acceptable salt, solvate, hydrate, tautomer, or stereoisomer thereof.

In some embodiments, for Formula (I), A is bicyclic heteroaryl (e.g., 8-fluoro-2-methylimidazo[1,2-a]pyridinyl); B is monocyclic heterocyclyl (e.g., 1,2,6-trimethylpiperazinyl); L¹ and L² are absent; X and Y are N; Z is C(R^{5a}) (e.g., —CH—); m is 0; and n is 0. In some embodiments, the compound of Formula (I) is Compound 824, 826, 912, 913, 914, or a pharmaceutically acceptable salt, solvate, hydrate, tautomer, or stereoisomer thereof.

In some embodiments, for Formula (I), A is bicyclic heteroaryl (e.g., 2-methylimidazo[1,2-a]pyridyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 827, 832, 915, 916, 917, or a pharmaceutically acceptable salt, solvate, hydrate, tautomer, or stereoisomer thereof.

In some embodiments, for Formula (I), A is bicyclic heteroaryl (e.g., 8-fluoro-2-(hydroxymethyl)imidazo[1,2-a]pyridinyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 828, 834, 918, 919, 920, or a pharmaceutically acceptable salt, solvate, hydrate, tautomer, or stereoisomer thereof.

In some embodiments, for Formula (I), A is bicyclic heteroaryl (e.g., 8-fluoro-2-(fluoromethyl)imidazo[1,2-a]pyridinyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 829, 835, 921, 922, 923, or a pharmaceutically acceptable salt, solvate, hydrate, tautomer, or stereoisomer thereof.

In some embodiments, for Formula (I), A is bicyclic heteroaryl (e.g., 3,7-difluoro-2-methyl-2H-indazolyl); B is monocyclic heterocyclyl (e.g., 2,6-dimethylpiperazinyl); L¹ and L² are absent; X and Y are N; Z is C(R^{5a}) (e.g., —CH—); m is 0; and n is 0. In some embodiments, the compound of Formula (I) is Compound 924, 925, 926, 927, 928, or a pharmaceutically acceptable salt, solvate, hydrate, tautomer, or stereoisomer thereof.

In some embodiments, for Formula (I), A is bicyclic heteroaryl (e.g., 6-hydroxy-7-fluoro-2-methyl-2H-indazolyl); B is monocyclic heterocyclyl (e.g., 3-(N-(tert-butyl)amino)pyrrolidinyl); L¹ and L² are absent; X and Y are N; Z is C(R^{5a}) (e.g., —CH—); m is 0; and n is 0. In some embodiments, the compound of Formula (I) is Compound 929, 930, 931, or a pharmaceutically acceptable salt, solvate, hydrate, tautomer, or stereoisomer thereof.

In some embodiments, the compound of Formula (II) is a compound of Formula (II-a):

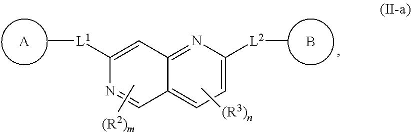

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁴ is independently hydrogen, C₁-C₆-alkyl, or C₁-C₆-haloalkyl; each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

In some embodiments, the compound of Formula (II) is a compound of Formula (II-b):

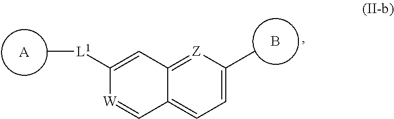

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and Z are each N or C(R⁶), wherein at least one of W and Z is N; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —N^{B}C(O)R^{D}—NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, —OR^{A}, —NR^{B}R^{C}, —N^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —R^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A}1; each R^{A1} is hydrogen or C₁-C₆-alkyl; m and n are each independently 0, 1, or 2; and x is 0, 1, or 2.

In some embodiments, the compound of Formula (II) is a compound of Formula (II-c):

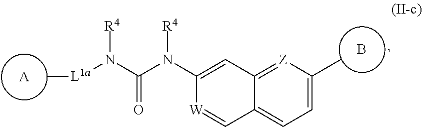

or a pharmaceutically acceptable salt, solvate, hydrate, tautomer, or stereoisomer thereof, wherein A and B are each independently cycloalkyl, heterocyclyl, aryl, or heteroaryl, each of which is optionally substituted with one or more R¹; W and Z are each N or C(R⁶), wherein at least one of W and Z is N; L^{1a} is absent or C₁-C₆-alkylene; each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷; each R² and R³ is independently C₁-C₆-alkyl, C₂-C₆-alkenyl, C₂-C₆-alkynyl, C₁-C₆-heteroalkyl, C₁-C₆-haloalkyl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}; each R⁶ is independently hydrogen, halo, C₁-C₆-alkyl, C₁-C₆-haloalkyl, or —OR^{A}; each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸; each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D}; each R^{B} and R^{C} is independently hydrogen, C₁-C₆ alkyl, C₁-C₆ heteroalkyl, cycloalkyl, heterocyclyl, or —OR^{A}; or R^{B} and R^{C} together with the atom to which they are attached form a 3-7-membered heterocyclyl ring optionally substituted with one or more R⁹; each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl; each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A}; each R⁹ is C₁-C₆-alkyl, halo, cyano, oxo, or —OR^{A1}; each R^{A1} is hydrogen or C₁-C₆-alkyl; m and n are each independently 0, 1, or 2; and x is 0, 1, or 2.

In some embodiments, the compound of Formula (II) is selected from a compound in Table 2, or a pharmaceutically acceptable salt, solvate, hydrate, tautomer, or stereoisomer thereof.

TABLE 2
Exemplary compounds of Formula (II)

| Compound | Ref. | Protocol/ | |
|---|---|---|---|
| No. | No. | Example | Structure |
| 110 | RMX-104 | Ex 4 | 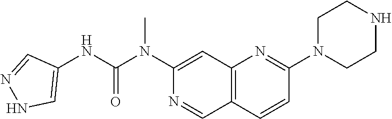 [SMILES: CN(C(=O)Nc1cn[nH]c1)c1cc2nc(N3CCNCC3)ccc2cn1] |
| 111 | RMX-85 | Ex 2 | 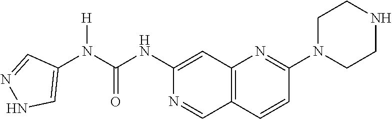 [SMILES: O=C(Nc1cn[nH]c1)Nc1cc2nc(N3CCNCC3)ccc2cn1] |
| 112 | RMX-97 | Ex 3 | 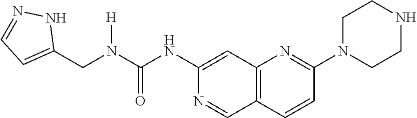 [SMILES: O=C(NCc1ccn[nH]1)Nc1cc2nc(N3CCNCC3)ccc2cn1] |

In some embodiments, for Formula (II), A is monocyclic heteroaryl (e.g., pyrazolyl); B is monocyclic heterocyclyl (e.g., piperazinyl); L¹ is —N(R⁴)C(O)N(R⁴)— (e.g., —NHC(O)N(CH₃)—); L² is absent; W and Z are N; m is 0; and n is 0. In some embodiments, the compound of Formula (I) is Compound 110, or a pharmaceutically acceptable salt, solvate, hydrate, tautomer, or stereoisomer thereof.

In some embodiments, for Formula (II), A is monocyclic heteroaryl (e.g., pyrazolyl); B is monocyclic heterocyclyl (e.g., piperazinyl); L¹ is —N(R⁴)C(O)N(R⁴)— (e.g., —NHC(O)NH—); L² is absent; W and Z are N; m is 0; and n is 0. In some embodiments, the compound of Formula (I) is Compound 111, or a pharmaceutically acceptable salt, solvate, hydrate, tautomer, or stereoisomer thereof.

In some embodiments, for Formula (II), A is monocyclic heteroaryl (e.g., pyrazolyl); B is monocyclic heterocyclyl (e.g., piperazinyl); L¹ is C₁-C₆ alkylene-N(R⁴)C(O)N(R⁴)— (e.g., —CH₂NHC(O)NH—); L² is absent; W and Z are N; m is 0; and n is 0. In some embodiments, the compound of Formula (I) is Compound 112, or a pharmaceutically acceptable salt, solvate, hydrate, tautomer, or stereoisomer thereof.

### Pharmaceutical Compositions, Kits, and Administration

The present invention provides pharmaceutical compositions comprising a compound of Formula (I) or (II), e.g., a compound of Formula (I) or (II) or a pharmaceutically acceptable salt, solvate, hydrate, tautomer, or stereoisomer, as described herein, and optionally a pharmaceutically acceptable excipient. In certain embodiments, the pharmaceutical composition described herein comprises a compound of Formula (I) or (II) or a pharmaceutically acceptable salt thereof, and optionally a pharmaceutically acceptable excipient. In certain embodiments, the compound of Formula (I) or (II) or a pharmaceutically acceptable salt, solvate, hydrate, tautomer, or stereoisomer thereof, is provided in an effective amount in the pharmaceutical composition. In certain embodiments, the effective amount is a therapeutically effective amount. In certain embodiments, the effective amount is a prophylactically effective amount.

Pharmaceutical compositions described herein can be prepared by any method known in the art of pharmacology. In general, such preparatory methods include the steps of bringing the compound of Formula (I) or (II) (the “active ingredient”) into association with a carrier and/or one or more other accessory ingredients, and then, if necessary and/or desirable, shaping and/or packaging the product into a desired single- or multi-dose unit.

Pharmaceutical compositions can be prepared, packaged, and/or sold in bulk, as a single unit dose, and/or as a plurality of single unit doses. As used herein, a “unit dose” is a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

Relative amounts of the active ingredient, the pharmaceutically acceptable excipient, and/or any additional ingredients in a pharmaceutical composition of the invention will vary, depending upon the identity, size, and/or condition of the subject treated and further depending upon the route by which the composition is to be administered. By way of example, the composition may comprise between 0.1% and 100% (w/w) active ingredient.

The term “pharmaceutically acceptable excipient” refers to a non-toxic carrier, adjuvant, diluent, or vehicle that does not destroy the pharmacological activity of the compound with which it is formulated. Pharmaceutically acceptable excipients useful in the manufacture of the pharmaceutical compositions of the invention are any of those that are well known in the art of pharmaceutical formulation and include inert diluents, dispersing and/or granulating agents, surface active agents and/or emulsifiers, disintegrating agents, binding agents, preservatives, buffering agents, lubricating agents, and/or oils. Pharmaceutically acceptable excipients useful in the manufacture of the pharmaceutical compositions of the invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

Compositions of the present invention may be administered orally, parenterally (including subcutaneous, intramuscular, intravenous and intradermal), by inhalation spray, topically, rectally, nasally, buccally, vaginally or via an implanted reservoir. In some embodiments, provided compounds or compositions are administrable intravenously and/or orally.

The term “parenteral” as used herein includes subcutaneous, intravenous, intramuscular, intraocular, intravitreal, intra-articular, intra-synovial, intrasternal, intrathecal, intrahepatic, intraperitoneal intralesional and intracranial injection or infusion techniques. Preferably, the compositions are administered orally, subcutaneously, intraperitoneally, or intravenously. Sterile injectable forms of the compositions of this invention may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium.

Pharmaceutically acceptable compositions of this invention may be orally administered in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient is combined with emulsifying and suspending agents. If desired, certain sweetening, flavoring or coloring agents may also be added. In some embodiments, a provided oral formulation is formulated for immediate release or sustained/delayed release. In some embodiments, the composition is suitable for buccal or sublingual administration, including tablets, lozenges and pastilles. A provided compound can also be in micro-encapsulated form.

Alternatively, pharmaceutically acceptable compositions of this invention may be administered in the form of suppositories for rectal administration. Pharmaceutically acceptable compositions of this invention may also be administered topically, especially when the target of treatment includes areas or organs readily accessible by topical application, including diseases of the eye, the skin, or the lower intestinal tract. Suitable topical formulations are readily prepared for each of these areas or organs.

For ophthalmic use, provided pharmaceutically acceptable compositions may be formulated as micronized suspensions or in an ointment such as petrolatum.

In order to prolong the effect of a drug, it is often desirable to slow the absorption of the drug from subcutaneous or intramuscular injection. This can be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Although the descriptions of pharmaceutical compositions provided herein are principally directed to pharmaceutical compositions which are suitable for administration to humans, it will be understood by the skilled artisan that such compositions are generally suitable for administration to animals of all sorts. Modification of pharmaceutical compositions suitable for administration to humans in order to render the compositions suitable for administration to various animals is well understood, and the ordinarily skilled veterinary pharmacologist can design and/or perform such modification with ordinary experimentation.

Compounds provided herein are typically formulated in dosage unit form, e.g., single unit dosage form, for ease of administration and uniformity of dosage. It will be understood, however, that the total daily usage of the compositions of the present invention will be decided by the attending physician within the scope of sound medical judgment. The specific therapeutically effective dose level for any particular subject or organism will depend upon a variety of factors including the disease being treated and the severity of the disorder; the activity of the specific active ingredient employed; the specific composition employed; the age, body weight, general health, sex and diet of the subject; the time of administration, route of administration, and rate of excretion of the specific active ingredient employed; the duration of the treatment; drugs used in combination or coincidental with the specific active ingredient employed; and like factors well known in the medical arts.

The exact amount of a compound required to achieve an effective amount will vary from subject to subject, depending, for example, on species, age, and general condition of a subject, severity of the side effects or disorder, identity of the particular compound(s), mode of administration, and the like. The desired dosage can be delivered three times a day, two times a day, once a day, every other day, every third day, every week, every two weeks, every three weeks, or every four weeks. In certain embodiments, the desired dosage can be delivered using multiple administrations (e.g., two, three, four, five, six, seven, eight, nine, ten, eleven, twelve, thirteen, fourteen, or more administrations).

In certain embodiments, an effective amount of a compound for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, of a compound per unit dosage form.

In certain embodiments, the compounds of Formula (I) or (II) may be at dosage levels sufficient to deliver from about 0.001 mg/kg to about 100 mg/kg, from about 0.01 mg/kg to about 50 mg/kg, preferably from about 0.1 mg/kg to about 40 mg/kg, preferably from about 0.5 mg/kg to about 30 mg/kg, from about 0.01 mg/kg to about 10 mg/kg, from about 0.1 mg/kg to about 10 mg/kg, and more preferably from about 1 mg/kg to about 25 mg/kg, of subject body weight per day, one or more times a day, to obtain the desired therapeutic effect.

It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult.

It will be also appreciated that a compound or composition, as described herein, can be administered in combination with one or more additional pharmaceutical agents. The compounds or compositions can be administered in combination with additional pharmaceutical agents that improve their bioavailability, reduce and/or modify their metabolism, inhibit their excretion, and/or modify their distribution within the body. It will also be appreciated that the therapy employed may achieve a desired effect for the same disorder, and/or it may achieve different effects.

The compound or composition can be administered concurrently with, prior to, or subsequent to, one or more additional pharmaceutical agents, which may be useful as, e.g., combination therapies. Pharmaceutical agents include therapeutically active agents. Pharmaceutical agents also include prophylactically active agents. Each additional pharmaceutical agent may be administered at a dose and/or on a time schedule determined for that pharmaceutical agent. The additional pharmaceutical agents may also be administered together with each other and/or with the compound or composition described herein in a single dose or administered separately in different doses. The particular combination to employ in a regimen will take into account compatibility of the inventive compound with the additional pharmaceutical agents and/or the desired therapeutic and/or prophylactic effect to be achieved. In general, it is expected that the additional pharmaceutical agents utilized in combination be utilized at levels that do not exceed the levels at which they are utilized individually. In some embodiments, the levels utilized in combination will be lower than those utilized individually.

Exemplary additional pharmaceutical agents include, but are not limited to, anti-proliferative agents, anti-cancer agents, anti-diabetic agents, anti-inflammatory agents, immunosuppressant agents, and a pain-relieving agent. Pharmaceutical agents include small organic molecules such as drug compounds (e.g., compounds approved by the U.S. Food and Drug Administration as provided in the Code of Federal Regulations (CFR)), peptides, proteins, carbohydrates, monosaccharides, oligosaccharides, polysaccharides, nucleoproteins, mucoproteins, lipoproteins, synthetic polypeptides or proteins, small molecules linked to proteins, glycoproteins, steroids, nucleic acids, DNAs, RNAs, nucleotides, nucleosides, oligonucleotides, antisense oligonucleotides, lipids, hormones, vitamins, and cells.

Also encompassed by the invention are kits (e.g., pharmaceutical packs). The inventive kits may be useful for preventing and/or treating a proliferative disease or a non-proliferative disease, e.g., as described herein. The kits provided may comprise an inventive pharmaceutical composition or compound and a container (e.g., a vial, ampule, bottle, syringe, and/or dispenser package, or other suitable container). In some embodiments, provided kits may optionally further include a second container comprising a pharmaceutical excipient for dilution or suspension of an inventive pharmaceutical composition or compound. In some embodiments, the inventive pharmaceutical composition or compound provided in the container and the second container are combined to form one-unit dosage form.

Thus, in one aspect, provided are kits including a first container comprising a compound described herein, or a pharmaceutically acceptable salt, solvate, hydrate, tautomer, or stereoisomer thereof, or a pharmaceutical composition thereof. In certain embodiments, the kit of the disclosure includes a first container comprising a compound described herein, or a pharmaceutically acceptable salt thereof, or a pharmaceutical composition thereof. In certain embodiments, the kits are useful in preventing and/or treating a disease, disorder, or condition described herein in a subject (e.g., a proliferative disease or a non-proliferative disease). In certain embodiments, the kits further include instructions for administering the compound, or a pharmaceutically acceptable salt, solvate, hydrate, tautomer, or stereoisomer thereof, or a pharmaceutical composition thereof, to a subject to prevent and/or treat a proliferative disease or a non-proliferative disease.

### Methods of Use

Described herein are compounds useful for modulating splicing. In some embodiments, a compound of Formula (I) or (II) may be used to alter the amount, structure, or composition of a nucleic acid (e.g., a precursor RNA, e.g., a pre-mRNA, or the resulting mRNA) by increasing or decreasing splicing at a splice site. In some embodiments, increasing or decreasing splicing results in modulating the level or structure of a gene product (e.g., an RNA or protein) produced. In some embodiments, a compound of Formula (I) or (II) may modulate a component of the splicing machinery, e.g., by modulating the interaction with a component of the splicing machinery with another entity (e.g., nucleic acid, protein, or a combination thereof). The splicing machinery as referred to herein comprises one or more spliceosome components. Spliceosome components may comprise, for example, one or more of major spliceosome members (U1, U2, U4, U5, U6 snRNPs), or minor spliceosome members (U11, U12, U4atac, U6atac snRNPs) and their accessory splicing factors.

In another aspect, the present disclosure features a method of modifying of a target (e.g., a precursor RNA, e.g., a pre-mRNA) through inclusion of a splice site in the target, wherein the method comprises providing a compound of Formula (I) or (II). In some embodiments, inclusion of a splice site in a target (e.g., a precursor RNA, e.g., a pre-mRNA, or the resulting mRNA) results in addition or deletion of one or more nucleic acids to the target (e.g., a new exon, e.g. a skipped exon). Addition or deletion of one or more nucleic acids to the target may result in an increase in the levels of a gene product (e.g., RNA, e.g., mRNA, or protein).

In another aspect, the present disclosure features a method of modifying a target (e.g., a precursor RNA, e.g., a pre-mRNA, or the resulting mRNA) through exclusion of a splice site in the target, wherein the method comprises providing a compound of Formula (I) or (II). In some embodiments, exclusion of a splice site in a target (e.g., a precursor RNA, e.g., a pre-mRNA) results in deletion or addition of one or more nucleic acids from the target (e.g., a skipped exon, e.g. a new exon). Deletion or addition of one or more nucleic acids from the target may result in a decrease in the levels of a gene product (e.g., RNA, e.g., mRNA, or protein). In other embodiments, the methods of modifying a target (e.g., a precursor RNA, e.g., a pre-mRNA, or the resulting mRNA) comprise suppression of splicing at a splice site or enhancement of splicing at a splice site (e.g., by more than about 0.5%, e.g., 1%, 5%, 10%, 15%, 20%, 25%, 30%, 35%, 40%, 45%, 50%, 55%, 60%, 65%, 70%, 75%, 80%, 85%, 90%, 95%, 99%, or more), e.g., as compared to a reference (e.g., the absence of a compound of Formula (I) or (II), or in a healthy or diseased cell or tissue).

The methods described herein can be used to modulate splicing, e.g., of a nucleic acid comprising a particular sequence (e.g., a target sequence). Exemplary genes encoding a target sequence (e.g., a target sequence comprising DNA or RNA, e.g., pre-mRNA) include, inter alia, ABCA4, ABCA9, ABCB1, ABCB5, ABCC9, ABCD1, ACADL, ACADM, ACADSB, ACSS2, ACTB, ACTG2, ADA, ADAL, ADAM10, ADAM15, ADAM22, ADAM32, ADAMTS12, ADAMTS13, ADAMTS20, ADAMTS6, ADAMTS9, ADAR, ADCY3, ADCY10, ADCY8, ADNP, ADRBK2, AFP, AGL, AGT, AHCTF1, AHR, AKAP10, AKAP3, AKNVA, ALAS1, ALS2CL, ALB, ALDH3A2, ALG6, AMBRA1, ANK3, ANTXR2, ANXA10, ANXA11, ANGPTL3, AP2A2, AP4E1, APC, APOA1, APOB, APOC3, APOH, AR, ARID2, ARID3A, ARID3B, ARFGEF1, ARFGEF2, ARHGAP1, ARHGAP8, ARHGAP18, ARHGAP26, ARHGEF18, ARHGEF2, ARPC3, ARS2, ASH1L, ASHIL-IT1, ASNSD1, ASPM, ATAD5, ATF1, ATG4A, ATG16L2, ATM, ATN1, ATP11C, ATP6VJG3, ATP13A5, ATP7A, ATP7B, ATR, ATXN2, ATXN3, ATXN7, ATXN10, AXIN1, B2M, B4GALNT3, BBS4, BCL2, BCL2L1, BCL2-like 11 (BIM), BCL11B, BBOXI, BCSIL, BEAN1, BHLHE40, BMPR2, BMP2K, BPTF, BRAF, BRCA1, BRCA2, BRCC3, BRSK1, BRSK2, BTAF1, BTK, C2orf55, C4orf29, C6orf118, C9orf43, C9orf72, C10orf137, C11orf30, C11orf65, C11orf70, C11orf87, C12orf51, C13orf1, C13orf15, C14orf11, C14orf118, C15orf29, C15orf42, C15orf60, C16orf33, C16orf38, C16orf48, C18orf8, C19orf42, C1orf107, C1orf114, C1orf130, C1orf149, C1orf27, C1orf71, C1orf94, C1R, C20orf74, C21orf70, C3orf23, C4orf18, C5orf34, C8B, C8orf33, C9orf114, C9orf86, C9orf98, C3, CA11, CAB39, CACHD1, CACNA1A, CACNA1B, CACNA1C, CACNA2D1, CACNA1G, CACNA1H, CALCA, CALCOCO2, CAMK1D, CAMKK1, CAPN3, CAPN9, CAPSL, CARD11, CARKD, CASZ1, CAT, CBLB, CBX1, CBX3, CCDC102B, CCDC11, CCDC15, CCDC18, CCDC5, CCDC81, CCDC131, CCDC146, CD4, CD274, CD1B, CDC14A, CDC16, CDC2L5, CDC42BPB, CDCA8, CDH10, CDH11, CDH24, CDH8, CDH9, CDK5RAP2, CDK6, CDK8, CDK11B, CD33, CD46, CDH1, CDH23, CDK6, CDK11B, CDK13, CEBPZ, CEL, CELSR3, CENPA, CENP1, CENPT, CENTB2, CENTG2, CEP110, CEP170, CEP192, CETP, CFB, CFTR, CFH, CGN, CGNL1, CHAF1A, CHD9, CHIC2, CHL1, CHN1, CHM, CLEC16A, CLIC2, CLCN1, CLINT1, CLK1, CLPB, CLPTM1, CMIP, CMYA5, CNGA3, CNOT1, CNOT7, CNTN6, COG3, COL11A1, COL11A2, COL12A1, COL14A1, COL15A1, COL17AM, COL19A1, COL1A1, COL1A2, COL2A, COL3A1, COL4A1, COL4A2, COL4A5, COL4A6, COL5A2, COL6A1, COL7A, COL9A, COL9A2, COL22A1, COL24A1, COL25A1, COL29A1, COLQ, COMTDI, COPA, COPB2, COPS7B, COPZ2, CPSF2, CPXM2, CR1, CRBN, CRYZ, CREBBP, CRKRS, CSE1L, CSTB, CSTF3, CT45-6, CTNNB1, CUBN, CUL4B, CUL5, CXorf41, CXXC1, CYBB, CYFIP2, CYP3A4, CYP3A43, CYP3A5, CYP4F2, CYP4F3, CYP17, CYP19, CYP24A1, CYP27A1, DAB1, DAZ2, DCBLD1, DCC, DCTN3, DCUN1D4, DDA1, DDEF1, DDX1, DDX24, DDX4, DENND2D, DEPDC2, DES, DGAT2, DHFR, DHRS7, DHRS9, DHX8, DIP2A, DMD, DMTF, DNAH3, DNAH8, DNA1J, DNAJA4, DNAJC3, DNAJC7, DNMT1, DNTTIP2, DOCK4, DOCK5, DOCKO, DOCK11, DOTIL, DPP3, DPP4, DPY19L2P2, DR1, DSCC1, DVL3, DUX4, DYNC1H1, DYSF, E2F1, E2F3, E2F8, E4F1, EBF1, EBF3, ECM2, EDEM3, EFCAB3, EFCAB4B, EFNA4, EFTUD2, EGFR, EIF3A, ELA1, ELA2A, ELF2, ELF3, ELF4, EMCN, EMD, EML5, ENO3, ENPP3, EP300, EPAS1, EPB41L5, EPHA3, EPHA4, EPHB1, EPHB2, EPHB3, EPS15, ERBB4, ERCC1, ERCC8, ERGIC3, ERMN, ERMP1, ERN1, ERN2, ESR1, ESRRG, ETS2, ETV3, ETV4, ETV5, ETV6, EVC2, EWSR1, EXO1, EXOC4, F3, F11, F13A1, F5, F7, F8, FAH, FAM13A1, FAM13B1, FAM13C1, FAM134A, FAM161A, FAM176B, FAM184A, FAM19A1, FAM20A, FAM23B, FAM65C, FANCA, FANCC, FANCG, FANCM, FANK1, FAR2, FBN1, FBXO15, FBX018, FBXO38, FCGBP, FECH, FEZ2, FGA, FGD6, FGFR2, FGFR10P, FGFR10P2, FGFR2, FGG, FGR, FIX FKBP3, FLI1, FLJ35848, FLJ36070, FLNA, FN1, FNBP1L, FOLH1, FOSL1, FOSL2, FOXK1, FOAM1, FOXO1, FOXP4, FRAS1, FUT9, FXN, FZD3, FZD6, GAB1, GABPA, GALC, GALNT3, GAPDH, GART, GAS2L3, GATA3, GATAD2A, GBA, GBGT1, GCG, GCGR, GCK, GFI1, GFM1, GH1, GHR, GHV, GJA1, GLA, GLT8D1, GNA11, GNAQ, GNAS, GNB5, GOLGB1, GOLTIA, GOLTIB, GPATCH1, GPR158, GPR160, GPX4, GRAMD3, GRHL1, GRHL2, GRHPR, GRIA1, GRIA3, GRIA4, GRIN2B, GRM3, GRM4, GRN, GSDMB, GSTCD, GSTO2, GTF2I, GTPBP4, HADHA, HAND2, HBA2, HBB, HCK, HDAC3, HDAC5, HDX, HEPACAM2, HERC1, HES7, HEXA, HEXB, HHEX, HIPK3, HLA-DPB1, HLA-G, HLCS, HLTF, HMBS, HMGA1, HMGCL, HNF1A, HNF1B, HNF4A, HNF4G, HNRNPH1, HOXCO, HP1BP3, HPGD, HPRT1, HPRT2, HSF1, HSF4, HSF2BP, HSPA9, HSPG2, HTT, HXA, ICA1, IDH1, IDS, IFI44L, IKBKAP, IKZF, IKZF3, IL1R2, IL5RA, IL7RA, IMA&LT, INPP5D, INSR, INTS3, INTU, IPO4, IP08, IQGAP2, IRF2, IRF4, IRF8, IRX3, ISL1, ISL2, ITFG1, ITGA6, ITGAL, ITGB1, ITGB2, ITGB3, ITGB4, ITIH1, ITPR2, IWS1, JAK1, JAK2, JAG1, JMJD1C, JPH3, KALRN, KAT6A, KATNAL2, KCNN2, KCNT2, KDM2A, KIAA0256, KIAA0528, KIAA0564, KIAA0586, KIAA1033, KIAA1166, KIAA1219, KIAA1409, KIAA1622, KIAA1787, KIF3B, KIF15, KIF16B, KIFSA, KIE5B, KIF9, KIN, KIR2DL5B, KIR3DL2, KIR3DL3, KIT, KLF3, KLF5, KLF7, KLFO, KLF12, KLF16, KLHL20, KLK12, KLKBI, KMT2A, KMT2B, KPNA5, KRAS, KREMENI, KRIT1, KRT5, KRTCAP2, KYNU, LICAM, L3MBTL, L3MBTL2, LACE1, LAMA1, LAMA2, LAMA3, LAMB1, LARP7, LDLR, LEF1, LENG1, LGALS3, LGMN, LHCGR, LHX3, LHX6, LIMCH1, LIMK2, LIN28B, LIN54, LMBRD1, LMBRD2, LMLN, LMNA, LMO2, LMO7, LOC389634, LOC390110, LPA, LPCAT2, LPL, LRP4, LRPPRC, LRRK2, LRRC19, LRRC42, LRWD1, LUM, LVRN, LYN, LYST, MADD, MAGI1, MAGT1, MALT1, MAP2K1, MAP4K4, MAPK8IP3, MAPK9, MAPT, MARC1, MARCH5, MATN2, MBD3, MCF2L2, MCM6, MDGA2, MDM4, ASXL1, FUS, SPR54, MECOM, MEF2C, MEF2D, MEGF10, MEGF1, MEMO1, MET, MGA, MGAM, MGAT4A, MGAT5, MGC16169, MGC34774, MKKS, MIB1, MIER2, MITF, MKL2, MLANA, MLH1, MLL5, MLX MME, MPDZ, MPI, MRAP2, MRPL11, MRPL39, MRPS28, MRPS35, MS4A13, MSH2, MSH3, MSMB, MST1R, MTDH, MTERF3, MTF1, MTF2, MTIF2, MTHFR, MUC2, MUT, MVK, MYB, MYBL2, MYC, MYCBP2, MYH2, MYRF, MYT1, MY019, MY03A, MY09B, MYOM2, MYOM3, NAG, NARG1, NARG2, NCOA1, NDC80, NDFIP2, NEB, NEDD4, NEK1, NEK5, NEK11, NF1, NF2, NFATC2, NFE2L2, NFIA, NFIB, NFIX, NFKB1, NFKB2, NFKBIL2, NFRKB, NFYA, NFYB, NIPA2, NKAIN2, NKAP, NLRC3, NLRC5, NLRP3, NLRP7, NLRP8, NLRP13, NME1, NME1-NME2, NME2, NME7, NOL10, NOP561, NOS1, NOS2A, NOTCH1, NPAS4, NPM1, NRID1, NRIH3, NRIH4, NR4A3, NR5A1, NRXNI, NSMAF, NSMCE2, NT5C, NT5C2, NT5C3, NUBP1, NUBPL, NUDT5, NUMA1, NUP88, NUP98, NUP160, NUPL1, OAT, OAZI, OBFC2A, OBFC2B, OLIG2, OMA1, OPA1, OPN4, OPTN, OSBPLI1, OSBPL8, OSGEPLI, OTC, OTX2, OVOL2, OXT, PA2G4, PAD14, PAH, PAN2, PAOX, PAPOLG, PARD3, PARPI, PARVB, PAWR, PAX3, PAX8, PBGD, PBRM1, PBX2, PCBP4, PCCA, PCGF2, PCNX, PCOTH, PDCD4, PDE4D, PDE8B, PDE10A, PD1A3, PDH1, PDLIM5, PDXK, PDZRN3, PEL12, PDK4, PDS5A, PDS5B, PGK1, PGM2, PHACTR4, PHEX, PHKB, PHLDB2, PHOX2B, PHTF1, PIAS1, PIEZO1, PIGF, PIGN, PIGT, PIK3C2G, PIK3CA, PIK3CD, PIK3CG, PIK3R1, PIP5K1A, PITRM1, PIWIL3, PKD1, PKHD1L1, PKD2, PKIB, PKLR, PKM1, PKM2, PLAGL2, PLCB1, PLCB4, PLCG1, PLD1, PLEKHA5, PLEKHA7, PLEKHM1, PLKR, PLXNC1, PMFBP1, POLN, POLR3D, POMT2, POSTN, POU2AF1, POU2F2, POU2F3, PPARA, PPFIA2, PPP1R12A, PPP3CB, PPP4C, PPP4RIL, PPP4R2, PRAME, PRC1, PRDM1, PREX1, PREX2, PRIM1, PRIM2, PRKAR1A, PRKCA, PRKG1, PRMT7, PROC, PROCR, PROSC, PRODH, PROX1, PRPF40B, PRPF4B, PRRG2, PRUNE2, PSD3, PSEN1, PSMAL, PTCH1, PTEN, PTK2, PTK2B, PTPN2, PTPN3, PTPN4, PTPN11, PTPN22, PTPRD, PTPRK, PTPRM, PTPRN2, PTPRT, PUS10, PVRL2, PYGM, QRSL1, RABI1FIP2, RAB23, RAF1, RALBP1, RALGDS, RBICCI, RBL2, RBM39, RBM45, RBP1, RBSN, REC8, RELB, RFC4, RFT1, RFTN1, RHOA, RHPN2, RIF1, RIT1, RLN3, RMND5B, RNFI1, RNF32, RNFTI, RNGTT, ROCK1, ROCK2, RORA, RP1, RP6KA3, RP11-265F1, RP13-36C9, RPAP3, RPNI, RPGR, RPL22, RPL22L1, RPS6KA6, RREBI, RRMI, RRPIB, RSK2, RTEL1, RTF1, RUFY1, RUNX1, RUNX2, RXRA, RYR3, SAALI, SAE1, SALL4, SAT1, SATB2, SBCAD, SCNIA, SCN2A, SCN3A, SCN4A, SCN5A, SCN8A, SCNA, SCN11A, SCO1, SCYL3, SDC1, SDK1, SDK2, SEC24A, SEC24D, SEC31A, SEL1L, SENP3, SENP6, SENP7, SERPINA1, SETD3, SETD4, SETDBI, SEZ6, SFRS12, SGCE, SGOL2, SGPLI, SH2DIA, SH3BGRL2, SH3PXD2A, SH3PXD2B, SH3RF2, SH3TC2, SHOC2, SIPA1L2, SIPA1L3, SIVA1, SKAP1, SKIV2L2, SLC6A11, SLC6A13, SLC6A6, SLC7A2, SLC12A3, SLC13A1, SLC22A17, SLC25A14, SLC28A3, SLC33A1, SLC35F6, SLC38A1, SLC38A4, SLC39A10, SLC4A2, SLC6A8, SMARCA1, SMARCA2, SMARCA5, SMARCC2, SMC5, SMN2, SMOX SMS, SMTN, SNCAIP, SNORD86, SNRK, SNRP70, SNX5, SNX6, SOD1, SODI0, SOS, SOS2, SOX5, SOX6, SOX8, SP1, SP2, SP3, SPH10, SPAG9, SPATA13, SPATA4, SPATS1, SPECC1L, SPDEF, SPI1, SPINK5, SPP2, SPTAI, SRF, SRM, SRP72, SSX3, SSX5, SSX9, STAG1, STAG2, STAMBPLI, STARD6, STAT1, STAT3, STAT5A, STAT5B, STAT6, STK17B, STX3, STXBPI, SUCLG2, SULF2, SUPT6H, SUPT16H, SV2C, SYCP2, SYT6, SYCP1, SYTL3, SYTL5, TAF2, TARDBP, TBCID3G, TBCID8B, TBCID26, TBCID29, TBCEL, TBK1, TBP, TBPL1, TBR1, TBX, TCEB3, TCF3, TCF4, TCF7L2, TCFL5, TCF12, TCP11L2, TDRD3, TEAD1, TEAD3, TEAD4, TECTB, TEK, TERF1, TERF2, TET2, TFAP2A, TFAP2B, TFAP2C, TFAP4, TFDP1, TFRC, TG, TGM7, TGSI, THAP7, THAP12, THOC2, TIAL1, TIAM2, TIMM50, TLK2, TM4SF20, TM6SF1, TMEM27, TMEM77, TMEM156, TMEM194A, TMFI, TMPRSS6, TNFRSF10A, TNFRSFOB, TNFRSF8, TNK2, TNKS, TNKS2, TOM1L1, TOM1L2, TOP2B, TP53, TP53INP1, TP53BP2, TP53I3, TP63, TRAF3IP3, TRAPPC2, TRIM44, TRIM65, TRIML1, TRIML2, TRPM3, TRPM5, TRPM7, TRPSI, TSC1, TSC2, TSHB, TSPAN7, TTC17, TTF1, TTLL5, TTLL9, TTN, TTPAL, TTR, TUSC3, TXNDCIO, UBE3A, UCK1, UGTIA1, UHRFIBPI, UNC45B, UNC5C, USH2A, USF2, USPI, USP6, USP18, USP38, USP39, UTP20, UTP15, UTP18, UTRN, UTX, UTY, UVRAG, UXT, VAPA, VEGFA, VPS29, VPS35, VPS39, VT11A, VT11B, VWA3B, WDFY2, WDR16, WDR17, WDR26, WDR44, WDR67, WDTC1, WRN, WRNIP1, WT1, WWC3, XBP1, XRN1, XRN2, XX—FW88277, YAP1, YARS, YBX1, YGM, YY1, ZBTB18, ZBTB20, ZC3HAV1, ZC3HC1, ZC3H7A, ZDHHC19, ZEB1, ZEB2, ZFPM1, ZFYVE1, ZFX ZIC2, ZNF37A, ZNF91, ZNF114, ZNF155, ZNF169, ZNF205, ZNF236, ZNF317, ZNF320, ZNF326, ZNF335, ZNF365, ZNF367, ZNF407, ZNF468, ZNF506, ZNF511, ZNF511-PRAP1, ZNF519, ZNF521, ZNF592, ZNF618, ZNF763, and ZWINT.

Additional exemplary genes encoding a target sequence (e.g., a target sequence comprising DNA or RNA, e.g., pre-mRNA) include genes include A1CF, A4GALT, AAR2, ABAT, ABCA11P, ZNF721, ABCA5, ABHD10, ABHD13, ABHD2, ABHD6, AC0000120.3, KRIT1, AC004076.1, ZNF772, AC004076.9, ZNF772, AC004223.3, RAD51D, AC004381.6, AC006486.1, ERF, AC0007390.5, AC0007780.1, PRKAR1A, AC0007998.2, INO80C, AC0009070.1, CMC2, AC009879.2, AC009879.3, ADHFE1, AC010487.3, ZNF816-ZNF321P, ZNF816, AC010328.3, AC010522.1, ZNF587B, AC010547.4, ZNF19, AC012313.3, ZNF497, AC012651.1, CAPN3, AC013489.1, DET1, AC016747.4, C2orf74, AC020907.6, FXYD3, AC021087.5, PDCD6, AHRR, AC022137.3, ZNF761, AC025283.3, NAA60, AC027644.4, RABGEF1, AC055811.2, FLCN, AC069368.3, ANKDD1A, AC073610.3, ARF3, AC074091.1, GPN1, AC079447.1, LIPT1, AC092587.1, AC079594.2, TRIM59, AC091060.1, C18orf21, AC092143.3, MC1R, AC093227.2, ZNF607, AC093512.2, ALDOA, AC098588.1, ANAPC10, AC107871.1, CALML4, AC114490.2, ZMYM6, AC138649.1, NIPA1, AC138894.1, CLN3, AC139768.1, AC242426.2, CHD1L, ACADM, ACAP3, ACKR2, RP11-141M3.5, KRBOX1, ACMSD, ACOT9, ACP5, ACPL2, ACSBG1, ACSF2, ACSF3, ACSL1, ACSL3, ACVR1, ADAL, ADAM29, ADAMTS10, ADAMTSL5, ADARB1, ADAT2, ADCK3, ADD3, ADGRG1, ADGRG2, ADHIB, ADIPOR1, ADNP, ADPRH, AGBL5, AGPATI, AGPAT3, AGR2, AGTR1, AHDC1, AHI1, AHNAK, AIFM1, AIFM3, AIMP2, AK4, AKAP1, AKNAD1, CLCC1, AKRIA1, AKT1, AKTISI, AKT2, AL139011.2, PEX19, AL157935.2, ST6GALNAC6, AL358113.1, TJP2, AL441992.2, KYAT1, AL449266.1, CLCC1, AL590556.3, LINC00339, CDC42, ALAS1, ALB, ALDH16A1, ALDHIB1, ALDH3A1, ALDH3B2, ALDOA, ALKBH2, ALPL, AMD1, AMICA1, AMN1, AMOTL2, AMY1B, AMY2B, ANAPC10, ANAPC11, ANAPC15, ANG, RNASE4, AL163636.2, ANGEL2, ANGPTL1, ANKMY1, ANKRD11, ANKRD28, ANKRD46, ANKRD9, ANKS3, ANKS3, RP11-127I20.7, ANKS6, ANKZF1, ANPEP, ANXA11, ANXA2, ANXA8L2, AL603965.1, AOC3, AP000304.12, CRYZ1, AP000311.1, CRYZ1, AP000893.2, RAB30, AP001267.5, ATP5MG, AP002495.2, AP003175.1, OR2AT4, AP003419.1, CLCF1, AP005263.1, ANKRD12, AP006621.5, AP006621.1, AP1G1, AP3M1, AP3M2, APBA2, APBB1, APLP2, APOA2, APOL1, APOL3, APTX, ARAP1, STARD10, ARF4, ARFIP1, ARFIP2, ARFRP1, ARHGAP11A, ARHGAP33, ARHGAP4, ARHGEF10, ARHGEF3, ARHGEF35, OR2A1-AS1, ARHGEF35, OR2A1-AS1, ARHGEF34P, ARID1B, ARHGEF35, OR2A20P, OR2A1-AS1, ARHGEF9, ARL1, ARL13B, ARL16, ARL6, ARMC6, ARMC8, ARMCX2, ARMCX5, RP4-769N13.6, ARMCX5-GPRASP2, BHLHB9, ARMCX5-GPRASP2, GPRASP1, ARMCX5-GPRASP2, GPRASP2, ARMCX6, ARNT2, ARPP19, ARRB2, ARSA, ART3, ASB3, GPR75-ASB3, ASCC2, ASNS, ASNS, AC079781.5, ASPSCR1, ASS1, ASUN, ATE1, ATF1, ATF7IP2, ATG13, ATG4D, ATG7, ATG9A, ATM, ATOX1, ATP1B3, ATP2C1, ATP5FIA, ATP5G2, ATP5J, ATP5MD, ATP5PF, ATP6AP2, ATP6VOB, ATP6V1C1, ATP6VID, ATP7B, ATXN1, ATXA1L, IST1, ATXA3, ATXA7L1, AURKA, AURKB, AXDND1, B3GALNT1, B3GALT5, AF064860.1, B3GALT5, AF064860.5, B3GNT5, B4GALT3, B4GALT4, B9D1, BACH1, BAIAP2, BANF1, BANF2, BAX, BAZ2A, BBIP1, BCHE, BCL2L14, BCL6, BCL9L, BCSIL, BDH1, BDKRB2, AL355102.2, BEST1, BEST3, BEX4, BHLHB9, BID, BIN3, BIRC2, BIVM, BIVM-ERCC5, BIVM, BLCAP, BLK, BLOCIS1, RP11-644F5.10, BLOCIS6, AC090527.2, BLOCIS6, RP11-96020.4, BLVRA, BMF, BOLA1, BORCS8-MEF2B, BORCS8, BRCA1, BRD1, BRDT, BRINP3, BROX BTBDIO, BTBD3, BTBD9, BTD, BTF3L4, BTNL9, BUBIB-PAK6, PAK6, BUB3, C10orf68, C11orf1, C11orf48, C11orf54, C11orf54, AP001273.2, C11orf57, C11orf63, C11orf82, C12orf23, C12orf4, C12orf65, C12orf79, C14orf159, C14orf93, C17orf62, C18orf21, C19orf12, C19orf40, C19orf47, C19orf48, C19orf54, C1D, C1GALT1, CIQB, CIQTNF1, CIS, C10rf101, C1orf112, C1orf116, C1orf159, C1orf63, C2, C2, CFB, C20orf27, C21orf58, C2CD4D, C2orf15, LIPT1, MRPL30, C2orf80, C2orf81, C3orf14, C3orf17, C3orf18, C3orf22, C3orf33, AC104472.3, C4orf33, C5orf28, C5orf34, C6orf118, C6orf203, C6orf211, C6orf48, C7orf50, C7orf55, C7orf55-LUC7L2, LUC7L2, C8orf44-SGK3, C8orf44, C8orf59, C9, DAB2, C9orf153, C9orf9, CA5BP1, CASB, CABYR, CALCA, CALCOCO1, CALCOCO2, CALM1, CALM3, CALML4, RP11-315D16.2, CALN, CALU, CANT1, CANX, CAP1, CAPN12, CAPS2, CARD8, CARHSP1, CARNS1, CASCI, CASP3, CASP7, CBFA2T2, CBS, CBY1, CCBL1, CCBL2, RBMXL1, CCDC12, CCDC126, CCDC14, CCDC149, CCDC150, CCDC169-SOHLH2, CCDC169, CCDC171, CCDC37, CCDC41, CCDC57, CCDC63, CCDC7, CCDC74B, CCDC77, CCDC82, CCDC90B, CCDC91, CCDC92, CCNE1, CCHCR1, CCL28, CCNB11P1, CCNC, CCND3, CCNG1, CCP11O, CCR9, CCT7, CCT8, CD151, CD1D, CD200, CD22, CD226, CD276, CD36, CD59, CDC26, CDC42, CDC42SEM, CDC42SE2, CDHR3, CDK10, CDK16, CDK4, CDKAL1, CDKL3, CTD-2410N18.4, CDKNIA, CDKN2A, CDNF, CEBPZOS, CELF1, CEMIP, CENPK, CEP170B, CEP250, CEP57, CEP57L1, CEP63, CERS4, CFL1, CFL2, CFLAR, CGNL1, CHCHD7, CHD1L, CHD8, CHFR, ZNF605, CHIA, CHID1, CHL1, CHM, CHMP1A, CHMP3, RNF103-CHMP3, CHRNA2, CIDEC, CIRBP, CITED1, CKLF-CMTM1, CMTM1, CKMT1B, CLDN12, CTB-13L3.1, CLDND1, AC021660.3, CLDND1, CPOX, CLHC1, CLIP1, CLUL1, CMC4, MTCPl, CNDP2, CNFN, CNOT1, CNOT6, CNOT7, CNOT8, CNR1, CNR2, CNTFR, CNTRL, COA1, COASY, COCH, COL8A1, COLCA1, COLEC11, COMMD3-BMI1, BMI1, COPS5, COPS7B, COQ8A, CORO6, COTL1, COX14, RP4-60503.4, COX7A2, COX7A2L, COX7B2, CPA4, CPA5, CPEB1, CPNE1, AL109827.1, RBM12, CPNE1, RP1-309K20.6, RBM12, CPNE3, CPSF3L, CPT1C, CREB3L2, CREM, CRP, CRYZ, CS, AC073896.1, CS, RP11-977G19.10, CSAD, CSDE1, CSF2RA, CSGALNACT1, CSK, CSNK2A1, CSRNP2, CT45A4, CT45A4, CT45A5, CT45A6, CTBP2, CTCFL, CTD-2116N17.1, KIAA0101, CTD-2349B8.1, SYT17, CTD-2528L19.4, ZNF607, CTD-2619J13.8, ZNF497, CTNNA1, CTNNBIP1, CTNND1, CTPS2, CTSB, CTSL, CTTN, CUL2, CUL9, CWC15, CXorf40B, CYB561A3, CYBC1, CYLD, CYP1LA1, CYP2R1, CYP4B1, CYP4F22, DAG1, DAGLB, KDELR2, DARS, DBNL, DCAF11, DCAF8, PEX19, DCLRE1C, DCTD, DCTN1, DCTN4, DCUNID2, DDR1, DDX11, DDX19B, AC012184.2, DDX19B, RP11-529K1.3, DDX25, DDX39B, ATP6V1G2-DDX39B, SNORD84, DDX42, DDX60L, DEDD, DEDD2, DEFA1, DEFAIB, DEFAIB, DEFA3, DENND1C, DENND2A, DENND4B, DET1, DGKA, DGKZ, DGLUCY, DHRS4L2, DHRS9, DHX40, DIABLO, AC048338.1, DIAPH1, DICER1, DKKL1, DLG1, DLG3, DLST, DMC1, DMKN, DMTF1, DMTN, DNAJC14, DNAJC19, DNAL1, DNASE1L1, DNMT3A, DOC2A, DOCK8, DOK1, DOPEY1, DPAGT1, DPP8, DRAM2, DRD2, DROSHA, DSN1, DTNA, DTX2, DTX3, DUOX1, DUOXA1, DUS2, DUSP10, DUSP13, DUSP18, DUSP22, DYDC1, DYDC2, DYNLL1, DYNLT1, DYRK1A, DYRK2, DYRK4, RPL1-500M8.7, DZIP1L, E2F6, ECHDC1, ECS1, ECT2, EDC3, EDEM1, EDEM2, MA&P24-AS1, RP4-61404.11, EEFIAKNMT, EEF1D, EFEMP1, EFHC1, EGFL7, EHF, EI24, EIF1AD, EIF2B5, EIF4G1, EIF2B5, POLR2H, EIF3E, EIF3K, EIF4E3, EIF4G1, ELF1, ELMO2, ELMOD1, AP000889.3, ELMOD3, ELOC, ELOF1, ELOVL, ELOVL7, ELP1, ELP6, EML3, EMP3, ENC1, ENDOV, ENO1, ENPP5, ENTHD2, ENTPD6, EP400NL, EPB41L1, EPDR1, NME8, EPHX1, EPM2A, EPN1, EPN2, EPN3, EPS8L2, ERBB3, ERC1, ERCC1, ERG, ERI2, ERI2, DCUN1D3, ERLIN2, ERMARD, ERRF11, ESR2, RP11-544I20.2, ESRRA, ESRRB, ESRRG, ETFA, ETFRF1, ETV1, ETV4, ETV7, EVA1A, EVC2, EVX1, EXD2, EXO5, EXOC1, EXOC2, FAAP24, FABP6, FADS1, FADS2, FAHD2B, FAM107B, FAM111A, FAMIIB, FAM114A1, FAM114A2, FAM115C, FAM115C, FAM115D, FAM120B, FAM133B, FAM135A, FAM153A, FAM153B, FAM154B, FAM156A, FAM156B, FAM168B, FAM172A, FAM182B, FAM192A, FAM19A2, FAM200B, FAM220A, FAM220A, AC009412.1, FAM222B, FAM227B, FAM234A, AC004754.1, FAM3C, FAM45A, FAM49B, FAM60A, FAM63A, FAM81A, FAM86B1, FAM86B2, FANCI, FANK1, FAR2, FAXC, FAXDC2, FBF1, FBH1, FBXL4, FBXO18, FBXO22, FBXO31, FBXO41, FBXO44, FBXO45, FBXW9, FCHOI, FCHSD2, FDFT1, FDPS, FER, FETUB, FGD4, FGF1, FGFR1, FGFRL1, FGL1, FHL2, FIBCD1, FIGNL1, FIGNL1, DDC, FKBP5, FKRP, FLRT2, FLRT3, FMC1, LUC7L2, FMC1-LUC7L2, FNDC3B, FOLH1, FOLR1, FOXP1, FOXK1, FOAM1, FOXO1, FOXP4, AC097634.4, FOXRED1, FPR1, FPR2, FRG1B, FRS2, FTO, FTSJ1, FUK, FUT10, FUT3, FUT6, FXYD3, FZD3, G2E3, GAA, GABARAPL1, GABPB1, GABRA5, GAL3ST1, GALE, GALNT11, GALNT14, GALNT6, GAPVD1, GARNL3, GAS2L3, GAS8, GATA1, GATA2, GATA4, GBA, GCNT1, GDPD2, GDPD5, GEMIN7, MARK4, GEMIN8, GGA3, GGACT, AL356966.1, GGPS1, GHRL, GID8, GIGYF2, GIMAP8, GIPC1, GJB1, GJB6, GLBIL, GLI1, GLT8D1, GMFG, GMPR2, GNAI2, GNAQ, GNB1, GNB2, GNE, GNG2, GNGT2, GNPDA1, GNPDA2, GOLGA3, CHFR, GOLGA4, GOLPH3L, GOLT1B, GPBP1L1, GPER1, GPR116, GPR141, EPDR1, GPR155, GPR161, GPR56, GPR63, GPR75-ASB3, ASB3, GPR85, GPSM2, GRAMD1B, GRB10, GRB7, GREM2, GRIA2, GSDMB, GSE1, GSN, GSTA4, GSTZ1, GTDC1, GTF2H1, GTF2H4, VARS2, GTF3C2, GUCY1A3, GUCYIB3, GUK1, GULP1, GYPC, GYS1, GZF1, HAGH, HA02, HAPLN3, HAVCR1, HAX1, HBG2, AC104389.4, HBG2, AC104389.4, HBE1, HBG2, AC104389.4, HBE1, OR51B5, HBG2, HBE1, AC104389.28, HBS1L, HCFC1R1, HCK, HDAC2, HDAC6, HDAC7, HDLBP, HEATR4, HECTD4, HEXIM2, HHAT, HHATL, CCDC13, HINFP, HIRA, C22orf39, HIVEP3, HJV, HKRI, HLF, HMBOXI, HMGA1, HMGB3, HMGCR, HMGN4, HMOX2, HNRNPC, HNRNPD, HNRNPH1, HNRNPH3, HNRNPR, HOMER3, HOPX HOXA3, HOXB3, HOXB3, HOXB4, HOXC4, HOXD3, HOXD3, HOXD4, HPCAL1, HPS4, HPS5, HRH1, HS3ST3A1, HSH2D, HSP90AA1, HSPD1, HTT, HUWE1, HYOU1, IAH1, ICA1L, ICAM2, ICE2, ICK, IDH2, IDH3G, IDS, IFI27, IFI44, IFT20, IFT22, IFT88, IGF2, INS-IGF2, IGF2BP3, IGFBP6, IKBKAP, IKBKB, IL11, IL18BP, IL18RAP, ILIRAP, IL1RL1, IL18R1, ILIRN, IL32, IL4I1, NUP62, AC011452.1, IL4I1, NUP62, CTC-326K19.6, IL6ST, ILVBL, IMAMP1L, IMPDH1, INCA1, ING1, INIP, INPP1, INPP51, INPP5K, INSIG2, INTS11, INTS12, INTS14, IP6K2, IP6K3, IPOI1, LRRC70, IQCE, IQGAP3, IRAK4, IRF3, IRF5, IRF6, ISG20, IST1, ISYNA1, ITFG2, ITGBIBP1, ITGB7, ITIH4, RP5-966M1.6, ITPRIPL1, JADE1, JAK2, JARID2, JDP2, KANK1, KANK1, RP11-31F19.1, KANK2, KANSL1L, KAT6A, KBTBD2, KBTBD3, KCNAB2, KCNE3, KCNG1, KCNJ16, KCNJ9, KCNMB2, AC117457.1, LINC01014, KCTD20, KCTD7, RABGEF1, KDMIB, KDM4A, AL451062.3, KHNYN, KIAA0040, KIAA0125, KIAA0196, KIAA0226L, PPPIR2P4, KIAA0391, KIAA0391, AL121594.1, KIAA0391, PSMA6, KIAA0753, KIAA0895, KIAA0895L, KIAA1191, KIAA1407, KIAA1841, C2orf74, KIF12, KIF14, KIF27, KIF9, KIFC3, KIN, KIRREL1, KITLG, KLC1, APOPT1, AL139300.1, KLC4, KLHDC4, KLHDC8A, KLHL13, KLHL18, KLHL2, KLHL24, KLHL7, KLKI1, KLK2, KLK5, KLK6, KLK7, KNOP1, KRBA2, AC135178.2, KRBA2, RP11-849F2.7, KRIT1, KRT15, KRT8, KTN1, KXD1, KYAT3, RBMXL1, KYNU, L3MBTL1, LACC1, LARGE, LARP4, LARP7, LAT2, LBHD1, LCA5, LCA5L, LCTL, LEPROTLI, LGALS8, LGALS9C, LGMN, LHFPL2, LIG4, LIMCH, LIMK2, LIMS2, LINC00921, ZNF263, LIPF, LLGL2, LMAN2L, LMCD1, LMF1, RP11-161M6.2, LMO1, LMO3, LOXHDI, LPAR1, LPAR2, LPAR4, LPAR5, LPAR6, LPHN1, LPIN2, LPIN3, LPP, LRFN5, LRIFI, LRMP, LRRC14, LRRC20, LRRC24, C8orf82, LRRC39, LRRC42, LRRC48, LRRC4C, LRRC8A, LRRC8B, LRRD1, LRTOMT, LRTOMT, AP000812.5, LSM7, LTB4R, LTBP3, LUC7L2, FMC1-LUC7L2, LUC7L3, LUZP1, LYG1, LYL1, LYPD4, LYPD6B, LYRM1, LYRM5, LYSMD4, MACC1, MAD1L1, MAD1L1, AC069288.1, MAEA, MAFF, MAFG, MAFK, MAGEA12, CSAG4, MAGEA2, MAGEA2B, MAGEA4, MAGEB1, MAGOHB, MAN2A2, MANBAL, MAOB, MAP2K3, MAP3K7CL, MAP3K8, MAP7, MAP9, MAPK6, MAPK7, MAPK8, MAPKAP1, 10-Mar, 7-Mar, 8-Mar, MARK2, MASP1, MATK, MATR3, MATR3, SNHG4, MB, MBD5, MBNL1, MBOAT7, MCC, MCFD2, MCM9, MCOLN3, MCRSI, MDC1, MDGA2, MDH2, MDM2, ME1, MEAK7, MECR, MED4, MEF2A, MEF2B, BORCS8-MEF2B, MEF2BNB-MEF2B, MEF2B, MEF2BNB, MEF2C, MEF2D, MEGFJO, MEl1, MEIS2, MELK, MET, METTL13, METTL23, MFF, MFN2, MFSD2A, MGST3, MIB2, MICAL1, MICAL3, MICOSO, NBL1, MICOS1O-NBL1, MID1, MINA, MINOSI-NBL1, MINOS1, MIOS, MIPOL1, MIS12, MKLN1, MKNKI, MKNKI, MOB3C, MLF2, MLH1, MMP17, MOBP, MOCS1, MOGS, MOK, MORF4L1, MPC1, MPC2, MPG, MPI, MPP1, MPP2, MPPE1, MPST, MRAS, MRO, MROH1, MROH7-TTC4, MROH7, MRPL14, MRPL24, MRPL33, BABAM2, MRPL33, BRE, MRPL47, MRPL48, MRPL55, MRRF, MRTFA, MRTFB, MRVI1, MS4A1, MS4A15, MS4A3, MS4A6E, MS4A7, MS4A14, MSANTD3, MSANTD4, MSH5, MSH5-SAPCD1, MSL2, MSRB3, MSS51, MTCP1, CMC4, MTERF, MTERF1, MTERF3, MTERFD2, MTERFD3, MTF2, MTG2, MTHFD2, MTHFD2L, MTIF2, MTIF3, MTMRI0, MTRF1, MTRR, MTUS2, MUTYH, MVK, MX1, MX2, MYH10, MYL12A, MYB, MYD88, MYL5, MYLIP, MYNN, MYOl5A, MYO1B, MYOM2, MZF1, N4BP2L2, NAA60, NAB1, NAE1, NAGK, NAP1L1, NAPIL4, NAPG, NARFL, NARG2, NAT1, NATIO, NBPFI1, WI2-3658N16.1, NBPF12, NBPF15, NBPF24, NBPF6, NBPF9, NBR1, NCAPG2, NCBP2, NCEH1, NCOA1, NCOA4, NDC1, NDRG1, NDRG2, NDRG4, NDST1, NDUFAF6, NDUFB2, NDUFC1, NDUFS1, NDUFS8, NDUFVI, NEDD1, NEIL1, NEIL2, NEK10, NEK11, NEK6, NEK9, NELFA, NEU4, NFAT5, NFE2, NFE2L2, AC019080.1, NFRKB, NFYA, NFYC, NIF3L1, NIPA2, NKIRAS1, NKX2-1, NLRC3, NME1, NME1-NME2, NME2, NME1-NME2, NME2, NME4, NME6, NME9, NOD1, NOL10, NOL8, NONO, NPAS1, NPIPA8, RP11-1212A22.1, NPIPB3, NPIPB4, NPIPB9, NPL, NPMJ, NPPA, NQO2, NRIH3, NR2C2, NR2F2, NR4A1, NRDC, NREP, NRF1, NRG4, NRIP1, NSD2, NSDHL, NSGI, NSMCE2, NSRPI, NT5C2, NTF4, NTMT1, NTNG2, NUBP2, NUCB2, NUDT1, NUDT2, NUDT4, NUF2, NUMBL, NUP50, NUP54, NUP85, NVL, NXF, NXPEI, NXPE3, OARD1, OAT, OAZ2, OCIAD1, OCLN, ODF2, OGDHL, OGFOD2, AC026362.1, OGFOD2, RP11-197N18.2, OLA1, OPRLI, OPTN, OR2H1, ORAI2, ORMDLI, ORMDL2, ORMDL3, OSBPL2, OSBPL3, OSBPL5, OSBPL9, OSER1, OSGIN1, OSR2, P2RX4, P2RY2, P2RY6, P4HA2, PABPC1, PACRGL, PACSIN3, PADI1, PAIP2, PAK1, PAK3, PAK4, PAK7, PALB2, PANK2, PAQR6, PARPH1, PARVG, PASK, PAX6, PBRM1, PBXIP1, PCBP3, PCBP4, AC115284.1, PCBP4, RPI1-155D18.14, RPI1-155D18.12, PCGF3, PCGF5, PCNP, PCSK9, PDCD10, PDCD6, AHRR, PDDC1, PDGFRB, PDIA6, PDIKIL, PDLIM7, PDP1, PDPK1, PDPN, PDZD11, PEA15, PEX2, PEX5, PEX5L, PFKM, PFN4, PGAP2, PGAP2, AC090587.2, PGAP3, PGM3, PGPEPI, PHB, PHC2, PHF20, PHF21A, PHF23, PHKB, PHLDB1, PHOSPHO1, PHOSPHO2, KLHL23, PI4 KB, PIAS2, PICALM, PIF1, PIGN, PIGO, PIGT, PIK3CD, PILRB, STAG3L5P-PVRIG2P-PILRB, PIP5K1B, PIR, PISD, PIWIL4, FUT4, PKD2, PKIA, PKIG, PKM, PKN2, PLA1A, PLA2G2A, PLA2G5, PLA2G7, PLAC8, PLAGL1, PLD1, PLD3, PLEKHAI, PLEKHA2, PLEKHA6, PLEKHG5, PLIN1, PLSI, PLS3, PLSCRI, PLSCR2, PLSCR4, PLXNBI, PLXNB2, PMP22, PMS1, PNISR, PNKP, AKTIS1, PNMT, PNPLA4, PNPLA8, PNPO, PNRCI, POCIB, POFUT1, POLB, POLD1, POLH, POLI, POLL, POLRIB, POM121, POM121C, AC006014.7, POM121C, AC211429.1, POMC, POMT1, POP1, PORCN, POU5F1, PSORSIC3, PPARD, PPARG, PPHLN, PPIL3, PPIL4, PPM1A, PPM1B, AC013717.1, PPPICB, PPPIRII, PPPIR13L, PPPIR26, PPPIR9A, PPP2R2B, PPP3CA, PPP6R1, PPP6R3, PPT2, PPT2-EGFL8, EGFL8, PPWD1, PRDM2, PRDM8, PRELID3A, PREPL, PRICKLE1, PRKAGI, PRMT2, PRMT5, PRMT7, PROM1, PRPS1, PRPSAP2, PRR14L, PRR15L, PRR5, PRR5-ARHGAP8, PRR5L, PRR7, PRRC2B, PRRT4, PRSS50, PRSS45, PRSS44, PRUNE, PRUNE1, PSEN1, PSMA2, PSMF1, PSORSIC1, PSPH, PSRC1, PTBP3, PTHLH, PTK2, PTPDC1, PTPRM, PUF60, PUM2, PUS1, PUS10, PXA, PXYLP1, PYCR1, QRICH1, R3HCCIL, R3HDM2, RAB17, RAB23, RAB3A, RAB3D, TMEM205, RAB4B-EGLN2, EGLN2, AC008537.1, RAB5B, RAB7L1, RABL2A, RABL2B, RABL5, RACGAPa, RAD17, RAD51L3-RFFL, RAD51D, RAD52, RAE1, RAI14, RAI2, RALBP1, RAN, RANGAP1, RAP1A, RAP1B, RAP1GAP, RAPGEF4, RAPGEFL1, RASGRP2, RASSF1, RBCK1, RBMI2B, RBM14, RBM4, RBM14-RBM4, RBM23, RBM4, RBM14-RBM4, RBM47, RBM7, AP002373.1, RBM7, RPI1-212D19.4, RBMS2, RBMYIE, RBP1, RBPMS, RBSN, RCBTB2, RCC1, RCC1, SNHG3, RCCD1, RECQL, RELL2, REPIN1, AC073111.3, REPIN1, ZNF775, RER1, RERE, RFWD3, RFX3, RGL2, RGMB, RGS11, RGS3, RGS5, AL592435.1, RHBDDI, RHNOI, TULP3, RHOC, AL603832.3, RHOC, RP11-426L16.10, RHOH, RIC8B, RIMKLB, RIN1, RIPK2, RIT1, RLIM, RNASE4, ANG, AL163636.6, RNASEK, RNASEK-C17orf49, RNF111, RNF123, RNF13, RNF14, RNF185, RNF216, RNF24, RNF32, RNF34, RNF38, RNF4, RNF44, RNH1, RNMT, RNPS1, R060, ROPN1, ROPN1B, ROR2, RP1-102H19.8, C6orf163, RP1-283E3.8, CDK11A, RP11-120M18.2, PRKAR1A, RP11-133K10.2, PAK6, RP11-164J13.1, CAPN3, RP11-21J18.1, ANKRD12, RP11-322E1.6, INO80C, RP11-337C18.10, CHDIL, RP1-432B6.3, TRIM59, RP11-468E2.4, IRF9, RP11-484M3.5, UPKIB, RP11-517H2.6, CCR6, RP1-613MI0.9, SLC25A51, RP11-659G9.3, RAB30, RP11-691N7.6, CTNND1, RP11-849H4.2, RP11-896JI0.3, NKX2-1, RPI1-96020.4, SQRDL, RP1-986E7.7, SERPINA3, RP4-769N13.6, GPRASP1, RP4-769N13.6, GPRASP2, RP4-798P15.3, SEC16B, RP5-10211I20.4, ZNF410, RP6-109B7.3, FLJ27365, RPE, RPH3AL, RPL15, RPL17, RPL17-C18orf32, RPL17, RPL23A, RPL36, HSDI1BIL, RPP38, RPS20, RPS27A, RPS3A, RPS6KA3, RPS6KC1, RPS6KL1, RPUSD1, RRAGD, RRAS2, RRBP1, RSL1D1, RSRC2, RSRP1, RUBCNL, RUNX1T1, RUVBL2, RWDD1, RWDD4, S100A13, AL162258.1, S100A13, RP1-178F15.5, S100A16, S100A4, S100A3, S100A6, S100PBP, SAA1, SACMIL, SAMD4B, SARIA, SARAF, SARNP, RP11-762I7.5, SCAMP5, SCAP, SCAPER, SCFD1, SCGB3A2, SCIN, SCML1, SCNNID, SCO2, SCOC, SCRNI, SDC2, SDC4, SEC13, SEC14L1, SEC14L2, SEC22C, SEC23B, SEC24C, SEC61G, SEMA4A, SEMA4C, SEMA4D, SEMA6C, SENP7, SEPPI, 11-Sep, 2-Sep, SERGEF, AC055860.1, SERP1, SERPINA1, SERPINA5, SERPINB6, SERPING1, SERPINH1, SERTAD3, SETD5, SFMBT1, AC096887.1, SFTPA1, SFTPA2, SFXN2, SGCD, SGCE, SGK3, SGK3, C8orf44, SH2B1, SH2D6, SH3BP1, Z83844.3, SH3BP2, SH3BP5, SH3D19, SH3YL1, SHC1, SHISA5, SHMT1, SHMT2, SHOC2, SHROOM1, SIGLEC5, SIGLEC14, SIL1, SIN3A, SIRT2, SIRT6, SKP1, STAT4, AC104109.3, SLAIN1, SLCI0A3, SLC12A9, SLC14A1, SLC16A6, SLCIA2, SLCIA6, SLC20A2, SLC25A18, SLC25A19, SLC25A22, SLC25A25, SLC25A29, SLC25A30, SLC25A32, SLC25A39, SLC25A44, SLC25A45, SLC25A53, SLC26A11, SLC26A4, SLC28A1, SLC29A1, SLC2A14, SLC2A5, SLC2A8, SLC35B2, SLC35B3, SLC35C2, SLC37A1, SLC38A1, SLC38A11, SLC39A13, SLC39A14, SLC41A3, SLC44A3, SLC4A7, SLC4A8, SLC5A10, SLC5A11, SLC6A1, SLC6A12, SLC6A9, SLC7A2, SLC7A6, SLC7A7, SLCO1A2, SLCO1C1, SLCO2B1, SLFNI1, SLFN12, SLFNL1, SLMO1, SLTM, SLU7, SMAD2, SMAP2, SMARCA2, SMARCE1, AC073508.2, SMARCE1, KRT222, SMC6, SMG7, SMIM22, SMOX, SMPDL3A, SMTN, SMU1, SMUG1, SNAP25, SNCA, SNRK, SNRPC, SNRPDl, SNRPD2, SNRPN, SNRPN, SNURF, SNUPN, SNXI1, SNX16, SNX17, SOATI, SOHLH2, CCDC169-SOHLH2, CCDC169, SORBSI, SORBS2, SOX5, SP2, SPART, SPATA20, SPATA21, SPATS2, SPATS2L, SPDYE2, SPECC1, SPECCIL, SPECCIL-ADORA2A, SPECCIL-ADORA2A, ADORA2A, SPEG, SPG20, SPG21, SPIDR, SPIN1, SPOCDI, SPOP, SPRR2A, SPRR2B, SPRR2E, SPRR2B, SPRR2F, SPRR2D, SPRR3, SPRY1, SPRY4, SPTBN2, SRC, SRGAP1, SRP68, SRSF11, SSX1, SSX2IP, ST3GAL4, ST3GAL6, ST5, ST6GALNAC6, ST7L, STAC3, STAG1, STAG2, STAMBP, STAMBPLI, STARD3NL, STAT6, STAU1, STAU2, AC022826.2, STAU2, RP11-463D19.2, STEAP2, STEAP3, STIL, STK25, STK33, STK38L, STK40, STMNI, STONI, STONI-GTF2AIL, STRAP, STRBP, STRC, AC011330.5, STRC, CATSPER2, STRC, CATSPER2, AC011330.5, STRC, STRCPI, STT3A, STX16-NPEPLI, NPEPL1, STX5, STX6, STX8, STXBP6, STYK1, SULTIAl, SULTIA2, SUMF2, SUN1, SUN2, SUN2, DNAL4, SUOX, SUPT6H, SUV39H2, SV2B, SYBU, SYNCRIP, SYNJ2, SYT1, SYTL4, TAB2, TACC1, TADA2B, TAFIC, TAF6, AC073842.2, TAF6, RP11-506M12.1, TAF9, TAGLN, TANK, TAPSARI, PSMB9, TAPT1, TATDN1, TAZ, TBC1D1, TBC1D12, HELLS, TBC1D15, TBCID3H, TBCID3G, TBC1D5, TBCID5, SATB1, TBCA, TBCEL, TBCEL, AP000646.1, TBL1XR1, TBP, TBX5, TBXAS1, TCAF1, TCEA2, TCEAL4, TCEAL8, TCEAL9, TCEANC, TCEB1, TCF19, TCF25, TCF4, TCP1, TCPIOL, AP000275.65, TCPI1, TCP11L2, TCTNI, TDG, TDP1, TDRD7, TEAD2, TECR, TENC1, TENT4A, TEX264, TEX30, TEX37, TFDP1, TFDP2, TFEB, TFG, TFP1, TF, TFP1, TGIF1, THAP6, THBS3, THOC5, THRAP3, THUMPD3, TIAL1, TIMM9, TIMP1, TIRAP, TJAP1, TJP2, TK2, TLDC1, TLE3, TLE6, TLN1, TLRIO, TM9SF1, TMBIMI, TMBIM4, TMBIM6, TMC6, TMCCI, TMCO4, TMEM126A, TMEM139, TMEM150B, TMEM155, TMEM161B, TMEM164, TMEM168, TMEM169, TMEM175, TMEM176B, TMEM182, TMEM199, CTB-96E2.3, TMEM216, TMEM218, TMEM230, TMEM263, TMEM45A, TMEM45B, TMEM62, TMEM63B, TMEM66, TMEM68, TMEM98, TMEM9B, TMPRSS11D, TMPRSS5, TMSB15B, TMTC4, TMUB2, TMX2-CTNND1, RP11-691N7.6, CTNND1, TNFAIP2, TNFAIP8L2, SCNM1, TNFRSF10C, TNFRSF9, TNFRSF8, TNFSF12-TNFSF3, TNFSF2, TNFSF3, TNFSF12-TNFSF3, TNFSF3, TNIPI, TNK2, TNNT1, TNRC18, TNS3, TOB2, TOM1L1, TOP1MT, TOP3B, TOX2, TP53, RPJJ-199F11.2, TP5311, TP53INP2, TPCN1, TPM3P9, AC022137.3, TPT1, TRA2B, TRAF2, TRAF3, TRAPPC12, TRAPPC3, TREH, TREX1, TREX2, TRIB2, TRIM3, TRIM36, TRIM39, TRIM46, TRIM6, TRIM6-TRIM34, TRIM6-TRIM34, TRIM34, TRIM66, TRIM73, TRIT1, TRMTIOB, TRMT2B, TRMT2B-AS1, TRNT1, TRO, TROVE2, TRPS1, TRPT1, TSC2, TSGA10, TSPANJ4, TSPAN3, TSPAN4, TSPAN5, TSPAN6, TSPAN9, TSPO, TTCJ2, TTC23, TTC3, TTC39A, TTC39C, TTLL1, TTLL7, TTPAL, TUBD1, TWNK, TXNL4A, TXNL4B, TXNRD1, TYK2, U2AF1, UBA2, UBA52, UBAP2, UBE2D2, UBE2D3, UBE2E3, UBE2I, UBE2J2, UBE3A, UBL7, UBXNJ1, UBXN7, UGDH, UGGTI, UGP2, UMAD1, AC007161.3, UNC45A, UQCCI, URGCP-MRPS24, URGCP, USMG5, USP16, USP21, USP28, USP3, USP33, USP35, USP54, USP9Y, USPLI, UTP15, VARS2, VASH2, VAV3, VDACl, VDAC2, VDR, VEZT, VGF, VILI, VILL, VIPRI, VPS29, VPS37C, VPS8, VPS9D1, VRK2, VWAI, VWA5A, WARS, WASFI, WASHC5, WBP5, WDHDI, WDPCP, WDR37, WDR53, WDR6, WDR72, WDR74, WDR81, WDR86, WDYHV1, WFDC3, WHSC1, WIPF1, WSCD2, WWP2, XAGE1A, XAGE1B, XKR9, XPNPEP1, ARCC3, XRN2, XXYLT1, YIF1A, YIF1B, YIPF1, YIPF5, YPEL5, YWHAB, YWHAZ, YYJAP1, ZBTB1, ZBTB14, ZBTB18, ZBTB20, ZBTB21, ZBTB25, ZBTB33, ZBTB34, ZBTB38, ZBTB43, ZBTB49, ZBTB7B, ZBTB7C, ZBTB8OS, ZC3HJJA, ZBED6, ZC3H13, ZCCHC17, ZCCHC7, ZDHHC11, ZDHHCJ3, ZEB2, ZFAND5, ZFAND6, ZFPI, ZFP62, ZFX, ZFYVE16, ZFYVE19, ZFYVE20, ZFYVE27, ZHX2, AC016405.1, ZHX3, ZIK1, ZIM2, PEG3, ZKSCAN1, ZKSCAN3, ZKSCAN8, ZMAT3, ZMAT5, ZMIZ2, ZMYM6, ZMYNDII, ZNFIO, AC026786.1, ZNF33, ZNF146, ZNF16, ZNF77, ZNF18, ZNF200, ZNF202, ZNF211, ZNF219, ZNF226, ZNF227, ZNF23, AC010547.4, ZNF23, AC010547.9, ZNF239, ZNF248, ZNF25, ZNF253, ZNF254, ZNF254, AC092279.1, ZNF263, ZNF274, ZNF275, ZNF28, ZNF468, ZNF283, ZNF287, ZNF3, ZNF320, ZNF322, ZNF324B, ZNF331, ZNF334, ZNF34, ZNF350, ZNF385A, ZNF395, FBXOJ6, ZNF415, ZNF418, ZNF43, ZNF433-AS1, AC0008770.4, ZNF438, ZNF444, ZNF445, ZNF467, ZNF480, ZNF493, ZNF493, CTD-2561J22.3, ZNF502, ZNF507, ZNF512, AC074091.1, ZNF512, RP1-158I13.2, ZNF512B, ZNF512B, SAMDIO, ZNF521, ZNF532, ZNF544, AC020915.5, ZNF544, CTD-3138B18.4, ZNF559, ZNF177, ZNF562, ZNF567, ZNF569, ZNF570, ZNF571-AS1, ZNF540, ZNF577, ZNF580, ZNF581, ZNF580, ZNF581, CCDC106, ZNF600, ZNF611, ZNF613, ZNF615, ZNF619, ZNF620, ZNF639, ZNF652, ZNF665, ZNF667, ZNF668, ZNF671, ZNF682, ZNF687, ZNF691, ZNF696, ZNF701, ZNF706, ZNF707, ZNF714, ZNF717, ZNF718, ZNF720, ZNF721, ZNF730, ZNF763, ZNF780B, AC005614.5, ZNF782, ZNF786, ZNF79, ZNF791, ZNF81, ZNF83, ZNF837, ZNF839, ZNF84, ZNF845, ZNF846, ZNF865, ZNF91, ZNF92, ZNHIT3, ZSCAN21, ZSCAN25, ZSCAN30, and ZSCAN32.

In some embodiments, the gene encoding a target sequence comprises the HTT gene. In some embodiments, the gene encoding a target sequence comprises the MYB gene. In some embodiments, the gene encoding a target sequence comprises the SMN2 gene. In some embodiments, the gene encoding a target sequence comprises the FOM1 gene.

Exemplary genes that may be modulated by the compounds of Formula (I) or (II) described herein may also include, inter alia, AC005258.1, AC005943.1, AC007849.1, AC008770.2, AC010487.3, AC011477.4, AC012651.1, AC012531.3, AC034102.2, AC073896.4, AC104472.3, AL109811.3, AL133342.1, AL137782.1, AL157871.5, AF241726.2, AL355336.1, AL358113.1, AL360181.3, AL445423.2, AL691482.3, AP001267.5, RF01169, and RF02271.

The compounds described herein may further be used to modulate a sequence comprising a particular splice site sequence, e.g., an RNA sequence (e.g., a pre-mRNA sequence). In some embodiments, the splice site sequence comprises a 5′ splice site sequence. In some embodiments, the splice site sequence comprises a 3′ splice site sequence. Exemplary gene sequences and splice site sequences (e.g., 5′ splice site sequences) include AAAgcaaguu (SEQ ID NO: 1), AAAguaaaaa (SEQ ID NO: 2), AAAguaaaau (SEQ ID NO: 3), AAAguaaagu (SEQ ID NO: 4), AAAguaaaua (SEQ ID NO: 5), AAAguaaaug (SEQ ID NO: 6), AAAguaaauu (SEQ ID NO: 7), AAAguaacac (SEQ ID NO: 8), AAAguaacca (SEQ ID NO: 9), AAAguaacuu (SEQ ID NO: 10), AAAguaagaa (SEQ ID NO: 11), AAAguaagac (SEQ ID NO: 12), AAAguaagag (SEQ ID NO: 13), AAAguaagau (SEQ ID NO: 14), AAAguaagca (SEQ ID NO: 15), AAAguaagcc (SEQ ID NO: 16), AAAguaagcu (SEQ ID NO: 17), AAAguaagga (SEQ ID NO: 18), AAAguaaggg (SEQ ID NO: 19), AAAguaaggu (SEQ ID NO: 20), AAAguaagua (SEQ ID NO: 21), AAAguaaguc (SEQ ID NO: 22), AAAguaagug (SEQ ID NO: 23), AAAguaaguu (SEQ ID NO: 24), AAAguaaucu (SEQ ID NO: 25), AAAguaauua (SEQ ID NO: 26), AAAguacaaa (SEQ ID NO: 27), AAAguaccgg (SEQ ID NO: 28), AAAguacuag (SEQ ID NO: 29), AAAguacugg (SEQ ID NO: 30), AAAguacuuc (SEQ ID NO: 31), AAAguacuug (SEQ ID NO: 32), AAAguagcuu (SEQ ID NO: 33), AAAguaggag (SEQ ID NO: 34), AAAguaggau (SEQ ID NO: 35), AAAguagggg (SEQ ID NO: 36), AAAguaggua (SEQ ID NO: 37), AAAguaguaa (SEQ ID NO: 38), AAAguauauu (SEQ ID NO: 39), AAAguauccu (SEQ ID NO: 40), AAAguaucuc (SEQ ID NO: 41), AAAguaugga (SEQ ID NO: 42), AAAguaugua (SEQ ID NO: 43), AAAguaugug (SEQ ID NO: 44), AAAguauguu (SEQ ID NO: 45), AAAguauugg (SEQ ID NO: 46), AAAguauuuu (SEQ ID NO: 47), AAAgucagau (SEQ ID NO: 48), AAAgucugag (SEQ ID NO: 49), AAAgugaaua (SEQ ID NO: 50), AAAgugagaa (SEQ ID NO: 51), AAAgugagac (SEQ ID NO: 52), AAAgugagag (SEQ ID NO: 53), AAAgugagau (SEQ ID NO: 54), AAAgugagca (SEQ ID NO: 55), AAAgugagcu (SEQ ID NO: 56), AAAgugaggg (SEQ ID NO: 57), AAAgugagua (SEQ ID NO: 58), AAAgugaguc (SEQ ID NO: 59), AAAgugagug (SEQ ID NO: 60), AAAgugaguu (SEQ ID NO: 61), AAAgugcguc (SEQ ID NO: 62), AAAgugcuga (SEQ ID NO: 63), AAAguggguc (SEQ ID NO: 64), AAAguggguu (SEQ ID NO: 65), AAAgugguaa (SEQ ID NO: 66), AAAguguaug (SEQ ID NO: 67), AAAgugugug (SEQ ID NO: 68), AAAguguguu (SEQ ID NO: 69), AAAguuaagu (SEQ ID NO: 70), AAAguuacuu (SEQ ID NO: 71), AAAguuagug (SEQ ID NO: 72), AAAguuaugu (SEQ ID NO: 73), AAAguugagu (SEQ ID NO: 74), AAAguuugua (SEQ ID NO: 75), AACguaaaac (SEQ ID NO: 76), AACguaaagc (SEQ ID NO: 77), AACguaaagg (SEQ ID NO: 78), AACguaagca (SEQ ID NO: 79), AACguaaggg (SEQ ID NO: 80), AACguaaguc (SEQ ID NO: 81), AACguaagug (SEQ ID NO: 82), AACguaaugg (SEQ ID NO: 83), AACguaguga (SEQ ID NO: 84), AACguaugua (SEQ ID NO: 85), AACguauguu (SEQ ID NO: 86), AACgugagca (SEQ ID NO: 87), AACgugagga (SEQ ID NO: 88), AACgugauuu (SEQ ID NO: 89), AACgugggau (SEQ ID NO: 90), AACgugggua (SEQ ID NO: 91), AACguguguu (SEQ ID NO: 92), AACguuggua (SEQ ID NO: 93), AAGgcaaauu (SEQ ID NO: 94), AAGgcaagag (SEQ ID NO: 95), AAGgcaagau (SEQ ID NO: 96), AAGgcaagcc (SEQ ID NO: 97), AAGgcaagga (SEQ ID NO: 98), AAGgcaaggg (SEQ ID NO: 99), AAGgcaagug (SEQ ID NO: 100), AAGgcaaguu (SEQ ID NO: 101), AAGgcacugc (SEQ ID NO: 102), AAGgcagaaa (SEQ ID NO: 103), AAGgcaggau (SEQ ID NO: 104), AAGgcaggca (SEQ ID NO: 105), AAGgcaggga (SEQ ID NO: 106), AAGgcagggg (SEQ ID NO: 107), AAGgcaggua (SEQ ID NO: 108), AAGgcaggug (SEQ ID NO: 109), AAGgcaucuc (SEQ ID NO: 110), AAGgcaugcu (SEQ ID NO: 111), AAGgcaugga (SEQ ID NO: 112), AAGgcauguu (SEQ ID NO: 113), AAGgcauuau (SEQ ID NO: 114), AAGgcgagcu (SEQ ID NO: 115), AAGgcgaguc (SEQ ID NO: 116), AAGgcgaguu (SEQ ID NO: 117), AAGgcuagcc (SEQ ID NO: 118), AAGguaaaaa (SEQ ID NO: 119), AAGguaaaac (SEQ ID NO: 120), AAGguaaaag (SEQ ID NO: 121), AAGguaaaau (SEQ ID NO: 122), AAGguaaaca (SEQ ID NO: 123), AAGguaaacc (SEQ ID NO: 124), AAGguaaacu (SEQ ID NO: 125), AAGguaaaga (SEQ ID NO: 126), AAGguaaagc (SEQ ID NO: 127), AAGguaaagg (SEQ ID NO: 128), AAGguaaagu (SEQ ID NO: 129), AAGguaaaua (SEQ ID NO: 130), AAGguaaauc (SEQ ID NO: 131), AAGguaaaug (SEQ ID NO: 132), AAGguaaauu (SEQ ID NO: 133), AAGguaacaa (SEQ ID NO: 134), AAGguaacau (SEQ ID NO: 135), AAGguaaccc (SEQ ID NO: 136), AAGguaacua (SEQ ID NO: 137), AAGguaacuc (SEQ ID NO: 138), AAGguaacug (SEQ ID NO: 139), AAGguaacuu (SEQ ID NO: 140), AAGguaagaa (SEQ ID NO: 141), AAGguaagac (SEQ ID NO: 142), AAGguaagag (SEQ ID NO: 143), AAGguaagau (SEQ ID NO: 144), AAGguaagca (SEQ ID NO: 145), AAGguaagcc (SEQ ID NO: 146), AAGguaagcg (SEQ ID NO: 147), AAGguaagcu (SEQ ID NO: 148), AAGguaagga (SEQ ID NO: 149), AAGguaaggc (SEQ ID NO: 150), AAGguaaggg (SEQ ID NO: 151), AAGguaaggu (SEQ ID NO: 152), AAGguaagua (SEQ ID NO: 153), AAGguaaguc (SEQ ID NO: 154), AAGguaagug (SEQ ID NO: 155), AAGguaaguu (SEQ ID NO: 156), AAGguaauaa (SEQ ID NO: 157), AAGguaauac (SEQ ID NO: 158), AAGguaauag (SEQ ID NO: 159), AAGguaauau (SEQ ID NO: 160), AAGguaauca (SEQ ID NO: 161), AAGguaaucc (SEQ ID NO: 162), AAGguaaucu (SEQ ID NO: 163), AAGguaauga (SEQ ID NO: 164), AAGguaaugc (SEQ ID NO: 165), AAGguaaugg (SEQ ID NO: 166), AAGguaaugu (SEQ ID NO: 167), AAGguaauua (SEQ ID NO: 168), AAGguaauuc (SEQ ID NO: 169), AAGguaauug (SEQ ID NO: 170), AAGguaauuu (SEQ ID NO: 171), AAGguacaaa (SEQ ID NO: 172), AAGguacaag (SEQ ID NO: 173), AAGguacaau (SEQ ID NO: 174), AAGguacacc (SEQ ID NO: 175), AAGguacacu (SEQ ID NO: 176), AAGguacagg (SEQ ID NO: 177), AAGguacagu (SEQ ID NO: 178), AAGguacaua (SEQ ID NO: 179), AAGguacaug (SEQ ID NO: 180), AAGguacauu (SEQ ID NO: 181), AAGguaccaa (SEQ ID NO: 182), AAGguaccag (SEQ ID NO: 183), AAGguaccca (SEQ ID NO: 184), AAGguacccu (SEQ ID NO: 185), AAGguaccuc (SEQ ID NO: 186), AAGguaccug (SEQ ID NO: 187), AAGguaccuu (SEQ ID NO: 188), AAGguacgaa (SEQ ID NO: 189), AAGguacggg (SEQ ID NO: 190), AAGguacggu (SEQ ID NO: 191), AAGguacguc (SEQ ID NO: 192), AAGguacguu (SEQ ID NO: 193), AAGguacuaa (SEQ ID NO: 194), AAGguacuau (SEQ ID NO: 195), AAGguacucu (SEQ ID NO: 196), AAGguacuga (SEQ ID NO: 197), AAGguacugc (SEQ ID NO: 198), AAGguacugu (SEQ ID NO: 199), AAGguacuuc (SEQ ID NO: 200), AAGguacuug (SEQ ID NO: 201), AAGguacuuu (SEQ ID NO: 202), AAGguagaaa (SEQ ID NO: 203), AAGguagaac (SEQ ID NO: 204), AAGguagaca (SEQ ID NO: 205), AAGguagacc (SEQ ID NO: 206), AAGguagacu (SEQ ID NO: 207), AAGguagagu (SEQ ID NO: 208), AAGguagaua (SEQ ID NO: 209), AAGguagcaa (SEQ ID NO: 210), AAGguagcag (SEQ ID NO: 211), AAGguagcca (SEQ ID NO: 212), AAGguagccu (SEQ ID NO: 213), AAGguagcua (SEQ ID NO: 214), AAGguagcug (SEQ ID NO: 215), AAGguagcuu (SEQ ID NO: 216), AAGguaggaa (SEQ ID NO: 217), AAGguaggag (SEQ ID NO: 218), AAGguaggau (SEQ ID NO: 219), AAGguaggca (SEQ ID NO: 220), AAGguaggcc (SEQ ID NO: 221), AAGguaggcu (SEQ ID NO: 222), AAGguaggga (SEQ ID NO: 223), AAGguagggc (SEQ ID NO: 224), AAGguagggg (SEQ ID NO: 225), AAGguagggu (SEQ ID NO: 226), AAGguaggua (SEQ ID NO: 227), AAGguagguc (SEQ ID NO: 228), AAGguaggug (SEQ ID NO: 229), AAGguagguu (SEQ ID NO: 230), AAGguaguaa (SEQ ID NO: 231), AAGguaguag (SEQ ID NO: 232), AAGguagucu (SEQ ID NO: 233), AAGguagugc (SEQ ID NO: 234), AAGguagugg (SEQ ID NO: 235), AAGguaguuc (SEQ ID NO: 236), AAGguaguuu (SEQ ID NO: 237), AAGguauaaa (SEQ ID NO: 238), AAGguauaau (SEQ ID NO: 239), AAGguauaca (SEQ ID NO: 240), AAGguauacu (SEQ ID NO: 241), AAGguauaua (SEQ ID NO: 242), AAGguauauc (SEQ ID NO: 243), AAGguauaug (SEQ ID NO: 244), AAGguauauu (SEQ ID NO: 245), AAGguaucac (SEQ ID NO: 246), AAGguaucag (SEQ ID NO: 247), AAGguauccc (SEQ ID NO: 248), AAGguauccu (SEQ ID NO: 249), AAGguaucuc (SEQ ID NO: 250), AAGguaucug (SEQ ID NO: 251), AAGguaucuu (SEQ ID NO: 252), AAGguaugaa (SEQ ID NO: 253), AAGguaugac (SEQ ID NO: 254), AAGguaugag (SEQ ID NO: 255), AAGguaugau (SEQ ID NO: 256), AAGguaugca (SEQ ID NO: 257), AAGguaugcc (SEQ ID NO: 258), AAGguaugcu (SEQ ID NO: 259), AAGguaugga (SEQ ID NO: 260), AAGguauggc (SEQ ID NO: 261), AAGguauggg (SEQ ID NO: 262), AAGguaugua (SEQ ID NO: 263), AAGguauguc (SEQ ID NO: 264), AAGguaugug (SEQ ID NO: 265), AAGguauguu (SEQ ID NO: 266), AAGguauuaa (SEQ ID NO: 267), AAGguauuac (SEQ ID NO: 268), AAGguauuag (SEQ ID NO: 269), AAGguauuau (SEQ ID NO: 270), AAGguauucc (SEQ ID NO: 271), AAGguauuga (SEQ ID NO: 272), AAGguauugu (SEQ ID NO: 273), AAGguauuua (SEQ ID NO: 274), AAGguauuuc (SEQ ID NO: 275), AAGguauuug (SEQ ID NO: 276), AAGguauuuu (SEQ ID NO: 277), AAGgucaaau (SEQ ID NO: 278), AAGgucaaga (SEQ ID NO: 279), AAGgucaagu (SEQ ID NO: 280), AAGgucacag (SEQ ID NO: 281), AAGgucagaa (SEQ ID NO: 282), AAGgucagac (SEQ ID NO: 283), AAGgucagag (SEQ ID NO: 284), AAGgucagca (SEQ ID NO: 285), AAGgucagcc (SEQ ID NO: 286), AAGgucagcg (SEQ ID NO: 287), AAGgucagcu (SEQ ID NO: 288), AAGgucagga (SEQ ID NO: 289), AAGgucaggc (SEQ ID NO: 290), AAGgucaggg (SEQ ID NO: 291), AAGgucaggu (SEQ ID NO: 292), AAGgucagua (SEQ ID NO: 293), AAGgucaguc (SEQ ID NO: 294), AAGgucagug (SEQ ID NO: 295), AAGgucaguu (SEQ ID NO: 296), AAGgucauag (SEQ ID NO: 297), AAGgucaucu (SEQ ID NO: 298), AAGguccaca (SEQ ID NO: 299), AAGguccaga (SEQ ID NO: 300), AAGguccaua (SEQ ID NO: 301), AAGgucccag (SEQ ID NO: 302), AAGgucccuc (SEQ ID NO: 303), AAGguccuuc (SEQ ID NO: 304), AAGgucgagg (SEQ ID NO: 305), AAGgucuaau (SEQ ID NO: 306), AAGgucuacc (SEQ ID NO: 307), AAGgucuaua (SEQ ID NO: 308), AAGgucuccu (SEQ ID NO: 309), AAGgucucug (SEQ ID NO: 310), AAGgucucuu (SEQ ID NO: 311), AAGgucugaa (SEQ ID NO: 312), AAGgucugag (SEQ ID NO: 313), AAGgucugga (SEQ ID NO: 314), AAGgucuggg (SEQ ID NO: 315), AAGgucugua (SEQ ID NO: 316), AAGgucuguu (SEQ ID NO: 317), AAGgucuucu (SEQ ID NO: 318), AAGgucuuuu (SEQ ID NO: 319), AAGgugaaac (SEQ ID NO: 320), AAGgugaaag (SEQ ID NO: 321), AAGgugaaau (SEQ ID NO: 322), AAGgugaacu (SEQ ID NO: 323), AAGgugaagc (SEQ ID NO: 324), AAGgugaagg (SEQ ID NO: 325), AAGgugaagu (SEQ ID NO: 326), AAGgugaaua (SEQ ID NO: 327), AAGgugaaug (SEQ ID NO: 328), AAGgugaauu (SEQ ID NO: 329), AAGgugacaa (SEQ ID NO: 330), AAGgugacag (SEQ ID NO: 331), AAGgugacau (SEQ ID NO: 332), AAGgugacug (SEQ ID NO: 333), AAGgugacuu (SEQ ID NO: 334), AAGgugagaa (SEQ ID NO: 335), AAGgugagac (SEQ ID NO: 336), AAGgugagag (SEQ ID NO: 337), AAGgugagau (SEQ ID NO: 338), AAGgugagca (SEQ ID NO: 339), AAGgugagcc (SEQ ID NO: 340), AAGgugagcg (SEQ ID NO: 341), AAGgugagcu (SEQ ID NO: 342), AAGgugagga (SEQ ID NO: 343), AAGgugaggc (SEQ ID NO: 344), AAGgugaggg (SEQ ID NO: 345), AAGgugaggu (SEQ ID NO: 346), AAGgugagua (SEQ ID NO: 347), AAGgugaguc (SEQ ID NO: 348), AAGgugagug (SEQ ID NO: 349), AAGgugaguu (SEQ ID NO: 350), AAGgugauaa (SEQ ID NO: 351), AAGgugauca (SEQ ID NO: 352), AAGgugaucc (SEQ ID NO: 353), AAGgugauga (SEQ ID NO: 354), AAGgugaugc (SEQ ID NO: 355), AAGgugaugu (SEQ ID NO: 356), AAGgugauua (SEQ ID NO: 357), AAGgugauug (SEQ ID NO: 358), AAGgugauuu (SEQ ID NO: 359), AAGgugcaca (SEQ ID NO: 360), AAGgugcauc (SEQ ID NO: 361), AAGgugcccu (SEQ ID NO: 362), AAGgugccug (SEQ ID NO: 363), AAGgugcgug (SEQ ID NO: 364), AAGgugcguu (SEQ ID NO: 365), AAGgugcucc (SEQ ID NO: 366), AAGgugcuga (SEQ ID NO: 367), AAGgugcugc (SEQ ID NO: 368), AAGgugcugg (SEQ ID NO: 369), AAGgugcuua (SEQ ID NO: 370), AAGgugcuuu (SEQ ID NO: 371), AAGguggaua (SEQ ID NO: 372), AAGguggcua (SEQ ID NO: 373), AAGguggcug (SEQ ID NO: 374), AAGguggcuu (SEQ ID NO: 375), AAGgugggaa (SEQ ID NO: 376), AAGgugggag (SEQ ID NO: 377), AAGgugggau (SEQ ID NO: 378), AAGgugggca (SEQ ID NO: 379), AAGgugggcc (SEQ ID NO: 380), AAGgugggcg (SEQ ID NO: 381), AAGgugggga (SEQ ID NO: 382), AAGguggggu (SEQ ID NO: 383), AAGgugggua (SEQ ID NO: 384), AAGgugggug (SEQ ID NO: 385), AAGguggguu (SEQ ID NO: 386), AAGgugguaa (SEQ ID NO: 387), AAGgugguac (SEQ ID NO: 388), AAGgugguau (SEQ ID NO: 389), AAGguggugg (SEQ ID NO: 390), AAGgugguua (SEQ ID NO: 391), AAGgugguuc (SEQ ID NO: 392), AAGgugguuu (SEQ ID NO: 393), AAGguguaag (SEQ ID NO: 394), AAGgugucaa (SEQ ID NO: 395), AAGgugucag (SEQ ID NO: 396), AAGgugucug (SEQ ID NO: 397), AAGgugugaa (SEQ ID NO: 398), AAGgugugag (SEQ ID NO: 399), AAGgugugca (SEQ ID NO: 400), AAGgugugga (SEQ ID NO: 401), AAGguguggu (SEQ ID NO: 402), AAGgugugua (SEQ ID NO: 403), AAGguguguc (SEQ ID NO: 404), AAGgugugug (SEQ ID NO: 405), AAGguguguu (SEQ ID NO: 406), AAGguguucu (SEQ ID NO: 407), AAGguguugc (SEQ ID NO: 408), AAGguguugg (SEQ ID NO: 409), AAGguguuug (SEQ ID NO: 410), AAGguuaaaa (SEQ ID NO: 411), AAGguuaaca (SEQ ID NO: 412), AAGguuaagc (SEQ ID NO: 413), AAGguuaauu (SEQ ID NO: 414), AAGguuacau (SEQ ID NO: 415), AAGguuagaa (SEQ ID NO: 416), AAGguuagau (SEQ ID NO: 417), AAGguuagca (SEQ ID NO: 418), AAGguuagcc (SEQ ID NO: 419), AAGguuagga (SEQ ID NO: 420), AAGguuaggc (SEQ ID NO: 421), AAGguuagua (SEQ ID NO: 422), AAGguuaguc (SEQ ID NO: 423), AAGguuagug (SEQ ID NO: 424), AAGguuaguu (SEQ ID NO: 425), AAGguuauag (SEQ ID NO: 426), AAGguuauga (SEQ ID NO: 427), AAGguucaaa (SEQ ID NO: 428), AAGguucaag (SEQ ID NO: 429), AAGguuccuu (SEQ ID NO: 430), AAGguucggc (SEQ ID NO: 431), AAGguucguu (SEQ ID NO: 432), AAGguucuaa (SEQ ID NO: 433), AAGguucuga (SEQ ID NO: 434), AAGguucuua (SEQ ID NO: 435), AAGguugaau (SEQ ID NO: 436), AAGguugacu (SEQ ID NO: 437), AAGguugagg (SEQ ID NO: 438), AAGguugagu (SEQ ID NO: 439), AAGguugaua (SEQ ID NO: 440), AAGguugcac (SEQ ID NO: 441), AAGguugcug (SEQ ID NO: 442), AAGguuggaa (SEQ ID NO: 443), AAGguuggca (SEQ ID NO: 444), AAGguuggga (SEQ ID NO: 445), AAGguugggg (SEQ ID NO: 446), AAGguuggua (SEQ ID NO: 447), AAGguugguc (SEQ ID NO: 448), AAGguuggug (SEQ ID NO: 449), AAGguugguu (SEQ ID NO: 450), AAGguuguaa (SEQ ID NO: 451), AAGguugucc (SEQ ID NO: 452), AAGguugugc (SEQ ID NO: 453), AAGguuguua (SEQ ID NO: 454), AAGguuuacc (SEQ ID NO: 455), AAGguuuaua (SEQ ID NO: 456), AAGguuuauu (SEQ ID NO: 457), AAGguuuccu (SEQ ID NO: 458), AAGguuucgu (SEQ ID NO: 459), AAGguuugag (SEQ ID NO: 460), AAGguuugca (SEQ ID NO: 461), AAGguuugcc (SEQ ID NO: 462), AAGguuugcu (SEQ ID NO: 463), AAGguuugga (SEQ ID NO: 464), AAGguuuggu (SEQ ID NO: 465), AAGguuugua (SEQ ID NO: 466), AAGguuuguc (SEQ ID NO: 467), AAGguuugug (SEQ ID NO: 468), AAGguuuuaa (SEQ ID NO: 469), AAGguuuuca (SEQ ID NO: 470), AAGguuuucg (SEQ ID NO: 471), AAGguuuugc (SEQ ID NO: 472), AAGguuuugu (SEQ ID NO: 473), AAGguuuuuu (SEQ ID NO: 474), AAUgcaagua (SEQ ID NO: 475), AAUgcaaguc (SEQ ID NO: 476), AAUguaaaca (SEQ ID NO: 477), AAUguaaaua (SEQ ID NO: 478), AAUguaaauc (SEQ ID NO: 479), AAUguaaaug (SEQ ID NO: 480), AAUguaaauu (SEQ ID NO: 481), AAUguaacua (SEQ ID NO: 482), AAUguaagaa (SEQ ID NO: 483), AAUguaagag (SEQ ID NO: 484), AAUguaagau (SEQ ID NO: 485), AAUguaagcc (SEQ ID NO: 486), AAUguaagcu (SEQ ID NO: 487), AAUguaagga (SEQ ID NO: 488), AAUguaagua (SEQ ID NO: 489), AAUguaaguc (SEQ ID NO: 490), AAUguaagug (SEQ ID NO: 491), AAUguaaguu (SEQ ID NO: 492), AAUguaauca (SEQ ID NO: 493), AAUguaauga (SEQ ID NO: 494), AAUguaaugu (SEQ ID NO: 495), AAUguacauc (SEQ ID NO: 496), AAUguacaug (SEQ ID NO: 497), AAUguacgau (SEQ ID NO: 498), AAUguacgua (SEQ ID NO: 499), AAUguacguc (SEQ ID NO: 500), AAUguacgug (SEQ ID NO: 501), AAUguacucu (SEQ ID NO: 502), AAUguaggca (SEQ ID NO: 503), AAUguagguu (SEQ ID NO: 504), AAUguaucua (SEQ ID NO: 505), AAUguaugaa (SEQ ID NO: 506), AAUguaugua (SEQ ID NO: 507), AAUguaugug (SEQ ID NO: 508), AAUguauguu (SEQ ID NO: 509), AAUgucagag (SEQ ID NO: 510), AAUgucagau (SEQ ID NO: 511), AAUgucagcu (SEQ ID NO: 512), AAUgucagua (SEQ ID NO: 513), AAUgucaguc (SEQ ID NO: 514), AAUgucagug (SEQ ID NO: 515), AAUgucaguu (SEQ ID NO: 516), AAUgucggua (SEQ ID NO: 517), AAUgucuguu (SEQ ID NO: 518), AAUgugagaa (SEQ ID NO: 519), AAUgugagca (SEQ ID NO: 520), AAUgugagcc (SEQ ID NO: 521), AAUgugagga (SEQ ID NO: 522), AAUgugagua (SEQ ID NO: 523), AAUgugaguc (SEQ ID NO: 524), AAUgugagug (SEQ ID NO: 525), AAUgugaguu (SEQ ID NO: 526), AAUgugauau (SEQ ID NO: 527), AAUgugcaua (SEQ ID NO: 528), AAUgugcgua (SEQ ID NO: 529), AAUgugcguc (SEQ ID NO: 530), AAUgugggac (SEQ ID NO: 531), AAUguggguc (SEQ ID NO: 532), AAUgugggug (SEQ ID NO: 533), AAUgugguuu (SEQ ID NO: 534), AAUgugugua (SEQ ID NO: 535), AAUguuaagu (SEQ ID NO: 536), AAUguuagaa (SEQ ID NO: 537), AAUguuagau (SEQ ID NO: 538), AAUguuagua (SEQ ID NO: 539), AAUguuggug (SEQ ID NO: 540), ACAgcaagua (SEQ ID NO: 541), ACAguaaaua (SEQ ID NO: 542), ACAguaaaug (SEQ ID NO: 543), ACAguaagaa (SEQ ID NO: 544), ACAguaagca (SEQ ID NO: 545), ACAguaagua (SEQ ID NO: 546), ACAguaaguc (SEQ ID NO: 547), ACAguaagug (SEQ ID NO: 548), ACAguaaguu (SEQ ID NO: 549), ACAguacgua (SEQ ID NO: 550), ACAguaggug (SEQ ID NO: 551), ACAguauaac (SEQ ID NO: 552), ACAguaugua (SEQ ID NO: 553), ACAgucaguu (SEQ ID NO: 554), ACAgugagaa (SEQ ID NO: 555), ACAgugagcc (SEQ ID NO: 556), ACAgugagcu (SEQ ID NO: 557), ACAgugagga (SEQ ID NO: 558), ACAgugaggu (SEQ ID NO: 559), ACAgugagua (SEQ ID NO: 560), ACAgugaguc (SEQ ID NO: 561), ACAgugagug (SEQ ID NO: 562), ACAgugaguu (SEQ ID NO: 563), ACAgugggua (SEQ ID NO: 564), ACAguggguu (SEQ ID NO: 565), ACAguguaaa (SEQ ID NO: 566), ACAguuaagc (SEQ ID NO: 567), ACAguuaagu (SEQ ID NO: 568), ACAguuaugu (SEQ ID NO: 569), ACAguugagu (SEQ ID NO: 570), ACAguuguga (SEQ ID NO: 571), ACCguaagua (SEQ ID NO: 572), ACCgugagaa (SEQ ID NO: 573), ACCgugagca (SEQ ID NO: 574), ACCgugaguu (SEQ ID NO: 575), ACCgugggug (SEQ ID NO: 576), ACGguaaaac (SEQ ID NO: 577), ACGguaacua (SEQ ID NO: 578), ACGguaagua (SEQ ID NO: 579), ACGguaagug (SEQ ID NO: 580), ACGguaaguu (SEQ ID NO: 581), ACGguaauua (SEQ ID NO: 582), ACGguaauuu (SEQ ID NO: 583), ACGguacaau (SEQ ID NO: 584), ACGguacagu (SEQ ID NO: 585), ACGguaccag (SEQ ID NO: 586), ACGguacggu (SEQ ID NO: 587), ACGguacgua (SEQ ID NO: 588), ACGguaggaa (SEQ ID NO: 589), ACGguaggag (SEQ ID NO: 590), ACGguaggug (SEQ ID NO: 591), ACGguaguaa (SEQ ID NO: 592), ACGguauaau (SEQ ID NO: 593), ACGguaugac (SEQ ID NO: 594), ACGguaugcg (SEQ ID NO: 595), ACGguaugua (SEQ ID NO: 596), ACGguauguc (SEQ ID NO: 597), ACGgugaaac (SEQ ID NO: 598), ACGgugaagu (SEQ ID NO: 599), ACGgugaauc (SEQ ID NO: 600), ACGgugacag (SEQ ID NO: 601), ACGgugacca (SEQ ID NO: 602), ACGgugagaa (SEQ ID NO: 603), ACGgugagau (SEQ ID NO: 604), ACGgugagcc (SEQ ID NO: 605), ACGgugagua (SEQ ID NO: 606), ACGgugagug (SEQ ID NO: 607), ACGgugaguu (SEQ ID NO: 608), ACGgugcgug (SEQ ID NO: 609), ACGguggcac (SEQ ID NO: 610), ACGguggggc (SEQ ID NO: 611), ACGgugggug (SEQ ID NO: 612), ACGguguagu (SEQ ID NO: 613), ACGgugucac (SEQ ID NO: 614), ACGgugugua (SEQ ID NO: 615), ACGguguguu (SEQ ID NO: 616), ACGguuagug (SEQ ID NO: 617), ACGguuaguu (SEQ ID NO: 618), ACGguucaau (SEQ ID NO: 619), ACUguaaaua (SEQ ID NO: 620), ACUguaagaa (SEQ ID NO: 621), ACUguaagac (SEQ ID NO: 622), ACUguaagca (SEQ ID NO: 623), ACUguaagcu (SEQ ID NO: 624), ACUguaagua (SEQ ID NO: 625), ACUguaaguc (SEQ ID NO: 626), ACUguaaguu (SEQ ID NO: 627), ACUguacguu (SEQ ID NO: 628), ACUguacuge (SEQ ID NO: 629), ACUguaggcu (SEQ ID NO: 630), ACUguaggua (SEQ ID NO: 631), ACUguauauu (SEQ ID NO: 632), ACUguaugaa (SEQ ID NO: 633), ACUguaugcu (SEQ ID NO: 634), ACUguaugug (SEQ ID NO: 635), ACUguauucc (SEQ ID NO: 636), ACUgucagcu (SEQ ID NO: 637), ACUgucagug (SEQ ID NO: 638), ACUgugaacg (SEQ ID NO: 639), ACUgugagca (SEQ ID NO: 640), ACUgugagcg (SEQ ID NO: 641), ACUgugagcu (SEQ ID NO: 642), ACUgugagua (SEQ ID NO: 643), ACUgugaguc (SEQ ID NO: 644), ACUgugagug (SEQ ID NO: 645), ACUgugaguu (SEQ ID NO: 646), ACUgugggua (SEQ ID NO: 647), ACUgugugug (SEQ ID NO: 648), ACUguuaagu (SEQ ID NO: 649), AGAgcaagua (SEQ ID NO: 650), AGAguaaaac (SEQ ID NO: 651), AGAguaaacg (SEQ ID NO: 652), AGAguaaaga (SEQ ID NO: 653), AGAguaaagu (SEQ ID NO: 654), AGAguaaauc (SEQ ID NO: 655), AGAguaaaug (SEQ ID NO: 656), AGAguaacau (SEQ ID NO: 657), AGAguaacua (SEQ ID NO: 658), AGAguaagaa (SEQ ID NO: 659), AGAguaagac (SEQ ID NO: 660), AGAguaagag (SEQ ID NO: 661), AGAguaagau (SEQ ID NO: 662), AGAguaagca (SEQ ID NO: 663), AGAguaagcu (SEQ ID NO: 664), AGAguaagga (SEQ ID NO: 665), AGAguaaggc (SEQ ID NO: 666), AGAguaaggg (SEQ ID NO: 667), AGAguaaggu (SEQ ID NO: 668), AGAguaaguc (SEQ ID NO: 669), AGAguaagug (SEQ ID NO: 670), AGAguaaguu (SEQ ID NO: 671), AGAguaauaa (SEQ ID NO: 672), AGAguaaugu (SEQ ID NO: 673), AGAguaauuc (SEQ ID NO: 674), AGAguaauuu (SEQ ID NO: 675), AGAguacacc (SEQ ID NO: 676), AGAguaccug (SEQ ID NO: 677), AGAguacgug (SEQ ID NO: 678), AGAguacucu (SEQ ID NO: 679), AGAguacuga (SEQ ID NO: 680), AGAguacuuu (SEQ ID NO: 681), AGAguagcug (SEQ ID NO: 682), AGAguaggaa (SEQ ID NO: 683), AGAguaggga (SEQ ID NO: 684), AGAguagggu (SEQ ID NO: 685), AGAguagguc (SEQ ID NO: 686), AGAguaggug (SEQ ID NO: 687), AGAguagguu (SEQ ID NO: 688), AGAguauaua (SEQ ID NO: 689), AGAguauauu (SEQ ID NO: 690), AGAguaugaa (SEQ ID NO: 691), AGAguaugac (SEQ ID NO: 692), AGAguaugau (SEQ ID NO: 693), AGAguauguc (SEQ ID NO: 694), AGAguaugug (SEQ ID NO: 695), AGAguauguu (SEQ ID NO: 696), AGAguauuaa (SEQ ID NO: 697), AGAguauuau (SEQ ID NO: 698), AGAgucagug (SEQ ID NO: 699), AGAgugagac (SEQ ID NO: 700), AGAgugagag (SEQ ID NO: 701), AGAgugagau (SEQ ID NO: 702), AGAgugagca (SEQ ID NO: 703), AGAgugagua (SEQ ID NO: 704), AGAgugaguc (SEQ ID NO: 705), AGAgugagug (SEQ ID NO: 706), AGAgugaguu (SEQ ID NO: 707), AGAgugcguc (SEQ ID NO: 708), AGAgugggga (SEQ ID NO: 709), AGAgugggug (SEQ ID NO: 710), AGAgugugug (SEQ ID NO: 711), AGAguguuuc (SEQ ID NO: 712), AGAguuagua (SEQ ID NO: 713), AGAguugaga (SEQ ID NO: 714), AGAguugagu (SEQ ID NO: 715), AGAguugguu (SEQ ID NO: 716), AGAguuugau (SEQ ID NO: 717), AGCguaagcu (SEQ ID NO: 718), AGCguaagug (SEQ ID NO: 719), AGCgugagcc (SEQ ID NO: 720), AGCgugagug (SEQ ID NO: 721), AGCguuguuc (SEQ ID NO: 722), AGGgcagagu (SEQ ID NO: 723), AGGgcagccu (SEQ ID NO: 724), AGGgcuagua (SEQ ID NO: 725), AGGguaaaga (SEQ ID NO: 726), AGGguaaaua (SEQ ID NO: 727), AGGguaaauc (SEQ ID NO: 728), AGGguaaauu (SEQ ID NO: 729), AGGguaacca (SEQ ID NO: 730), AGGguaacug (SEQ ID NO: 731), AGGguaacuu (SEQ ID NO: 732), AGGguaagaa (SEQ ID NO: 733), AGGguaagag (SEQ ID NO: 734), AGGguaagau (SEQ ID NO: 735), AGGguaagca (SEQ ID NO: 736), AGGguaagga (SEQ ID NO: 737), AGGguaaggc (SEQ ID NO: 738), AGGguaaggg (SEQ ID NO: 739), AGGguaagua (SEQ ID NO: 740), AGGguaaguc (SEQ ID NO: 741), AGGguaagug (SEQ ID NO: 742), AGGguaaguu (SEQ ID NO: 743), AGGguaauac (SEQ ID NO: 744), AGGguaauga (SEQ ID NO: 745), AGGguaauua (SEQ ID NO: 746), AGGguaauuu (SEQ ID NO: 747), AGGguacacc (SEQ ID NO: 748), AGGguacagu (SEQ ID NO: 749), AGGguacggu (SEQ ID NO: 750), AGGguaggac (SEQ ID NO: 751), AGGguaggag (SEQ ID NO: 752), AGGguaggca (SEQ ID NO: 753), AGGguaggcc (SEQ ID NO: 754), AGGguaggga (SEQ ID NO: 755), AGGguagggu (SEQ ID NO: 756), AGGguagguc (SEQ ID NO: 757), AGGguaggug (SEQ ID NO: 758), AGGguagguu (SEQ ID NO: 759), AGGguauaua (SEQ ID NO: 760), AGGguaugac (SEQ ID NO: 761), AGGguaugag (SEQ ID NO: 762), AGGguaugau (SEQ ID NO: 763), AGGguaugca (SEQ ID NO: 764), AGGguaugcu (SEQ ID NO: 765), AGGguauggg (SEQ ID NO: 766), AGGguauggu (SEQ ID NO: 767), AGGguaugua (SEQ ID NO: 768), AGGguauguc (SEQ ID NO: 769), AGGguaugug (SEQ ID NO: 770), AGGguauuac (SEQ ID NO: 771), AGGguauucu (SEQ ID NO: 772), AGGguauuuc (SEQ ID NO: 773), AGGgucagag (SEQ ID NO: 774), AGGgucagca (SEQ ID NO: 775), AGGgucagga (SEQ ID NO: 776), AGGgucaggg (SEQ ID NO: 777), AGGgucagug (SEQ ID NO: 778), AGGgucaguu (SEQ ID NO: 779), AGGguccccu (SEQ ID NO: 780), AGGgucggga (SEQ ID NO: 781), AGGgucugca (SEQ ID NO: 782), AGGgucuguu (SEQ ID NO: 783), AGGgugaaga (SEQ ID NO: 784), AGGgugacua (SEQ ID NO: 785), AGGgugagaa (SEQ ID NO: 786), AGGgugagac (SEQ ID NO: 787), AGGgugagag (SEQ ID NO: 788), AGGgugagca (SEQ ID NO: 789), AGGgugagcc (SEQ ID NO: 790), AGGgugagcu (SEQ ID NO: 791), AGGgugagga (SEQ ID NO: 792), AGGgugaggg (SEQ ID NO: 793), AGGgugaggu (SEQ ID NO: 794), AGGgugagua (SEQ ID NO: 795), AGGgugaguc (SEQ ID NO: 796), AGGgugagug (SEQ ID NO: 797), AGGgugaguu (SEQ ID NO: 798), AGGgugggga (SEQ ID NO: 799), AGGguggggu (SEQ ID NO: 800), AGGgugggua (SEQ ID NO: 801), AGGgugggug (SEQ ID NO: 802), AGGgugugua (SEQ ID NO: 803), AGGgugugug (SEQ ID NO: 804), AGGguuaaug (SEQ ID NO: 805), AGGguuagaa (SEQ ID NO: 806), AGGguuaguu (SEQ ID NO: 807), AGGguuggug (SEQ ID NO: 808), AGGguuugug (SEQ ID NO: 809), AGGguuuguu (SEQ ID NO: 810), AGUguaaaag (SEQ ID NO: 811), AGUguaaaua (SEQ ID NO: 812), AGUguaaauu (SEQ ID NO: 813), AGUguaagaa (SEQ ID NO: 814), AGUguaagag (SEQ ID NO: 815), AGUguaagau (SEQ ID NO: 816), AGUguaagca (SEQ ID NO: 817), AGUguaagcc (SEQ ID NO: 818), AGUguaagua (SEQ ID NO: 819), AGUguaagug (SEQ ID NO: 820), AGUguaaguu (SEQ ID NO: 821), AGUguaauug (SEQ ID NO: 822), AGUguaggac (SEQ ID NO: 823), AGUguagguc (SEQ ID NO: 824), AGUguaugag (SEQ ID NO: 825), AGUguaugua (SEQ ID NO: 826), AGUguauguu (SEQ ID NO: 827), AGUguauugu (SEQ ID NO: 828), AGUguauuua (SEQ ID NO: 829), AGUgucaguc (SEQ ID NO: 830), AGUgugagag (SEQ ID NO: 831), AGUgugagca (SEQ ID NO: 832), AGUgugagcc (SEQ ID NO: 833), AGUgugagcu (SEQ ID NO: 834), AGUgugagua (SEQ ID NO: 835), AGUgugaguc (SEQ ID NO: 836), AGUgugagug (SEQ ID NO: 837), AGUgugaguu (SEQ ID NO: 838), AGUgugggua (SEQ ID NO: 839), AGUgugggug (SEQ ID NO: 840), AGUgugugua (SEQ ID NO: 841), AGUguuccua (SEQ ID NO: 842), AGUguugggg (SEQ ID NO: 843), AGUguuucag (SEQ ID NO: 844), AUAguaaaua (SEQ ID NO: 845), AUAguaagac (SEQ ID NO: 846), AUAguaagau (SEQ ID NO: 847), AUAguaagca (SEQ ID NO: 848), AUAguaagua (SEQ ID NO: 849), AUAguaagug (SEQ ID NO: 850), AUAguaaguu (SEQ ID NO: 851), AUAguaggua (SEQ ID NO: 852), AUAguauguu (SEQ ID NO: 853), AUAgucucac (SEQ ID NO: 854), AUAgugagac (SEQ ID NO: 855), AUAgugagag (SEQ ID NO: 856), AUAgugagau (SEQ ID NO: 857), AUAgugagcc (SEQ ID NO: 858), AUAgugaggc (SEQ ID NO: 859), AUAgugagua (SEQ ID NO: 860), AUAgugaguc (SEQ ID NO: 861), AUAgugagug (SEQ ID NO: 862), AUAgugcguc (SEQ ID NO: 863), AUAgugugua (SEQ ID NO: 864), AUAguucagu (SEQ ID NO: 865), AUCguaagcc (SEQ ID NO: 866), AUCguaaguu (SEQ ID NO: 867), AUCguauucc (SEQ ID NO: 868), AUCgugagua (SEQ ID NO: 869), AUGgcaagcg (SEQ ID NO: 870), AUGgcaagga (SEQ ID NO: 871), AUGgcaaguu (SEQ ID NO: 872), AUGgcaggua (SEQ ID NO: 873), AUGgcaugug (SEQ ID NO: 874), AUGgcgccau (SEQ ID NO: 875), AUGgcuugug (SEQ ID NO: 876), AUGguaaaac (SEQ ID NO: 877), AUGguaaaau (SEQ ID NO: 878), AUGguaaacc (SEQ ID NO: 879), AUGguaaaga (SEQ ID NO: 880), AUGguaaaua (SEQ ID NO: 881), AUGguaaaug (SEQ ID NO: 882), AUGguaaauu (SEQ ID NO: 883), AUGguaacag (SEQ ID NO: 884), AUGguaacau (SEQ ID NO: 885), AUGguaacua (SEQ ID NO: 886), AUGguaacuc (SEQ ID NO: 887), AUGguaacuu (SEQ ID NO: 888), AUGguaagaa (SEQ ID NO: 889), AUGguaagac (SEQ ID NO: 890), AUGguaagag (SEQ ID NO: 891), AUGguaagau (SEQ ID NO: 892), AUGguaagca (SEQ ID NO: 893), AUGguaagcc (SEQ ID NO: 894), AUGguaagcu (SEQ ID NO: 895), AUGguaagga (SEQ ID NO: 896), AUGguaaggg (SEQ ID NO: 897), AUGguaagua (SEQ ID NO: 898), AUGguaaguc (SEQ ID NO: 899), AUGguaagug (SEQ ID NO: 900), AUGguaaguu (SEQ ID NO: 901), AUGguaauaa (SEQ ID NO: 902), AUGguaauau (SEQ ID NO: 903), AUGguaauga (SEQ ID NO: 904), AUGguaaugg (SEQ ID NO: 905), AUGguaauug (SEQ ID NO: 906), AUGguaauuu (SEQ ID NO: 907), AUGguacage (SEQ ID NO: 908), AUGguacauc (SEQ ID NO: 909), AUGguaccag (SEQ ID NO: 910), AUGguaccug (SEQ ID NO: 911), AUGguacgag (SEQ ID NO: 912), AUGguacggu (SEQ ID NO: 913), AUGguagauc (SEQ ID NO: 914), AUGguagcag (SEQ ID NO: 915), AUGguagcug (SEQ ID NO: 916), AUGguaggaa (SEQ ID NO: 917), AUGguaggau (SEQ ID NO: 918), AUGguaggca (SEQ ID NO: 919), AUGguaggcu (SEQ ID NO: 920), AUGguagggg (SEQ ID NO: 921), AUGguagggu (SEQ ID NO: 922), AUGguaggua (SEQ ID NO: 923), AUGguaggug (SEQ ID NO: 924), AUGguaguuu (SEQ ID NO: 925), AUGguauagu (SEQ ID NO: 926), AUGguauaua (SEQ ID NO: 927), AUGguaucag (SEQ ID NO: 928), AUGguaucuu (SEQ ID NO: 929), AUGguaugau (SEQ ID NO: 930), AUGguaugca (SEQ ID NO: 931), AUGguaugcc (SEQ ID NO: 932), AUGguaugcg (SEQ ID NO: 933), AUGguaugcu (SEQ ID NO: 934), AUGguaugga (SEQ ID NO: 935), AUGguauggc (SEQ ID NO: 936), AUGguaugug (SEQ ID NO: 937), AUGguauguu (SEQ ID NO: 938), AUGguauuau (SEQ ID NO: 939), AUGguauuga (SEQ ID NO: 940), AUGguauuug (SEQ ID NO: 941), AUGgucaggg (SEQ ID NO: 942), AUGgucaguc (SEQ ID NO: 943), AUGgucagug (SEQ ID NO: 944), AUGgucauuu (SEQ ID NO: 945), AUGgugaaaa (SEQ ID NO: 946), AUGgugaaac (SEQ ID NO: 947), AUGgugaaau (SEQ ID NO: 948), AUGgugaacu (SEQ ID NO: 949), AUGgugaaga (SEQ ID NO: 950), AUGgugacgu (SEQ ID NO: 951), AUGgugagaa (SEQ ID NO: 952), AUGgugagac (SEQ ID NO: 953), AUGgugagag (SEQ ID NO: 954), AUGgugagca (SEQ ID NO: 955), AUGgugagcc (SEQ ID NO: 956), AUGgugagcg (SEQ ID NO: 957), AUGgugagcu (SEQ ID NO: 958), AUGgugaggc (SEQ ID NO: 959), AUGgugaggg (SEQ ID NO: 960), AUGgugagua (SEQ ID NO: 961), AUGgugaguc (SEQ ID NO: 962), AUGgugagug (SEQ ID NO: 963), AUGgugaguu (SEQ ID NO: 964), AUGgugauuu (SEQ ID NO: 965), AUGgugcgau (SEQ ID NO: 966), AUGgugegug (SEQ ID NO: 967), AUGgugggua (SEQ ID NO: 968), AUGgugggug (SEQ ID NO: 969), AUGguggguu (SEQ ID NO: 970), AUGgugguua (SEQ ID NO: 971), AUGguguaag (SEQ ID NO: 972), AUGgugugaa (SEQ ID NO: 973), AUGgugugua (SEQ ID NO: 974), AUGgugugug (SEQ ID NO: 975), AUGguuacuc (SEQ ID NO: 976), AUGguuagca (SEQ ID NO: 977), AUGguuaguc (SEQ ID NO: 978), AUGguuagug (SEQ ID NO: 979), AUGguuaguu (SEQ ID NO: 980), AUGguucagu (SEQ ID NO: 981), AUGguucguc (SEQ ID NO: 982), AUGguuggua (SEQ ID NO: 983), AUGguugguc (SEQ ID NO: 984), AUGguugguu (SEQ ID NO: 985), AUGguuguuu (SEQ ID NO: 986), AUGguuugca (SEQ ID NO: 987), AUGguuugua (SEQ ID NO: 988), AUUgcaagua (SEQ ID NO: 989), AUUguaaaua (SEQ ID NO: 990), AUUguaagau (SEQ ID NO: 991), AUUguaagca (SEQ ID NO: 992), AUUguaagga (SEQ ID NO: 993), AUUguaaggc (SEQ ID NO: 994), AUUguaagua (SEQ ID NO: 995), AUUguaaguc (SEQ ID NO: 996), AUUguaaguu (SEQ ID NO: 997), AUUguaauua (SEQ ID NO: 998), AUUguaauuu (SEQ ID NO: 999), AUUguacaaa (SEQ ID NO: 1000), AUUguaccuc (SEQ ID NO: 1001), AUUguacgug (SEQ ID NO: 1002), AUUguacuug (SEQ ID NO: 1003), AUUguaggua (SEQ ID NO: 1004), AUUguaugag (SEQ ID NO: 1005), AUUguaugua (SEQ ID NO: 1006), AUUgucuguu (SEQ ID NO: 1007), AUUgugagcu (SEQ ID NO: 1008), AUUgugagua (SEQ ID NO: 1009), AUUgugaguc (SEQ ID NO: 1010), AUUgugaguu (SEQ ID NO: 1011), AUUgugcgug (SEQ ID NO: 1012), AUUgugggug (SEQ ID NO: 1013), AUUguuagug (SEQ ID NO: 1014), CAAguaaaaa (SEQ ID NO: 1015), CAAguaaaua (SEQ ID NO: 1016), CAAguaaauc (SEQ ID NO: 1017), CAAguaaaug (SEQ ID NO: 1018), CAAguaaccc (SEQ ID NO: 1019), CAAguaacua (SEQ ID NO: 1020), CAAguaacug (SEQ ID NO: 1021), CAAguaagaa (SEQ ID NO: 1022), CAAguaagac (SEQ ID NO: 1023), CAAguaagau (SEQ ID NO: 1024), CAAguaaggu (SEQ ID NO: 1025), CAAguaagua (SEQ ID NO: 1026), CAAguaaguc (SEQ ID NO: 1027), CAAguaagug (SEQ ID NO: 1028), CAAguaaguu (SEQ ID NO: 1029), CAAguaaucc (SEQ ID NO: 1030), CAAguaaucu (SEQ ID NO: 1031), CAAguaauua (SEQ ID NO: 1032), CAAguaauuc (SEQ ID NO: 1033), CAAguaauug (SEQ ID NO: 1034), CAAguaauuu (SEQ ID NO: 1035), CAAguacaca (SEQ ID NO: 1036), CAAguacguu (SEQ ID NO: 1037), CAAguacuuu (SEQ ID NO: 1038), CAAguagcug (SEQ ID NO: 1039), CAAguaggau (SEQ ID NO: 1040), CAAguaggua (SEQ ID NO: 1041), CAAguagguc (SEQ ID NO: 1042), CAAguaggug (SEQ ID NO: 1043), CAAguagguu (SEQ ID NO: 1044), CAAguaguuu (SEQ ID NO: 1045), CAAguauaac (SEQ ID NO: 1046), CAAguauaug (SEQ ID NO: 1047), CAAguaucuu (SEQ ID NO: 1048), CAAguaugag (SEQ ID NO: 1049), CAAguaugua (SEQ ID NO: 1050), CAAguauguc (SEQ ID NO: 1051), CAAguaugug (SEQ ID NO: 1052), CAAguauguu (SEQ ID NO: 1053), CAAguauuga (SEQ ID NO: 1054), CAAguauuuc (SEQ ID NO: 1055), CAAgucagac (SEQ ID NO: 1056), CAAgucagua (SEQ ID NO: 1057), CAAgucuaua (SEQ ID NO: 1058), CAAgucugau (SEQ ID NO: 1059), CAAgugacuu (SEQ ID NO: 1060), CAAgugagaa (SEQ ID NO: 1061), CAAgugagac (SEQ ID NO: 1062), CAAgugagca (SEQ ID NO: 1063), CAAgugaggc (SEQ ID NO: 1064), CAAgugaggg (SEQ ID NO: 1065), CAAgugagua (SEQ ID NO: 1066), CAAgugaguc (SEQ ID NO: 1067), CAAgugagug (SEQ ID NO: 1068), CAAgugaucc (SEQ ID NO: 1069), CAAgugaucu (SEQ ID NO: 1070), CAAgugauuc (SEQ ID NO: 1071), CAAgugauug (SEQ ID NO: 1072), CAAgugauuu (SEQ ID NO: 1073), CAAgugccuu (SEQ ID NO: 1074), CAAgugggua (SEQ ID NO: 1075), CAAguggguc (SEQ ID NO: 1076), CAAgugggug (SEQ ID NO: 1077), CAAgugugag (SEQ ID NO: 1078), CAAguuaaaa (SEQ ID NO: 1079), CAAguuaagu (SEQ ID NO: 1080), CAAguuaauc (SEQ ID NO: 1081), CAAguuagaa (SEQ ID NO: 1082), CAAguuaguu (SEQ ID NO: 1083), CAAguucaag (SEQ ID NO: 1084), CAAguuccgu (SEQ ID NO: 1085), CAAguuggua (SEQ ID NO: 1086), CAAguuuagu (SEQ ID NO: 1087), CAAguuucca (SEQ ID NO: 1088), CAAguuuguu (SEQ ID NO: 1089), CACguaagag (SEQ ID NO: 1090), CACguaagca (SEQ ID NO: 1091), CACguaauug (SEQ ID NO: 1092), CACguaggac (SEQ ID NO: 1093), CACguaucga (SEQ ID NO: 1094), CACgucaguu (SEQ ID NO: 1095), CACgugagcu (SEQ ID NO: 1096), CACgugaguc (SEQ ID NO: 1097), CACgugagug (SEQ ID NO: 1098), CAGgcaagaa (SEQ ID NO: 1099), CAGgcaagac (SEQ ID NO: 1100), CAGgcaagag (SEQ ID NO: 1101), CAGgcaagga (SEQ ID NO: 1102), CAGgcaagua (SEQ ID NO: 1103), CAGgcaagug (SEQ ID NO: 1104), CAGgcaaguu (SEQ ID NO: 1105), CAGgcacgca (SEQ ID NO: 1106), CAGgcagagg (SEQ ID NO: 1107), CAGgcaggug (SEQ ID NO: 1108), CAGgcaucau (SEQ ID NO: 1109), CAGgcaugaa (SEQ ID NO: 1110), CAGgcaugag (SEQ ID NO: 1111), CAGgcaugca (SEQ ID NO: 1112), CAGgcaugcg (SEQ ID NO: 1113), CAGgcaugug (SEQ ID NO: 1114), CAGgcgagag (SEQ ID NO: 1115), CAGgcgccug (SEQ ID NO: 1116), CAGgcgugug (SEQ ID NO: 1117), CAGguaaaaa (SEQ ID NO: 1118), CAGguaaaag (SEQ ID NO: 1119), CAGguaaaca (SEQ ID NO: 1120), CAGguaaacc (SEQ ID NO: 1121), CAGguaaaga (SEQ ID NO: 1122), CAGguaaagc (SEQ ID NO: 1123), CAGguaaagu (SEQ ID NO: 1124), CAGguaaaua (SEQ ID NO: 1125), CAGguaaauc (SEQ ID NO: 1126), CAGguaaaug (SEQ ID NO: 1127), CAGguaaauu (SEQ ID NO: 1128), CAGguaacag (SEQ ID NO: 1129), CAGguaacau (SEQ ID NO: 1130), CAGguaacca (SEQ ID NO: 1131), CAGguaaccg (SEQ ID NO: 1132), CAGguaacgu (SEQ ID NO: 1133), CAGguaacua (SEQ ID NO: 1134), CAGguaacuc (SEQ ID NO: 1135), CAGguaacug (SEQ ID NO: 1136), CAGguaacuu (SEQ ID NO: 1137), CAGguaagaa (SEQ ID NO: 1138), CAGguaagac (SEQ ID NO: 1139), CAGguaagag (SEQ ID NO: 1140), CAGguaagau (SEQ ID NO: 1141), CAGguaagcc (SEQ ID NO: 1142), CAGguaagga (SEQ ID NO: 1143), CAGguaaggc (SEQ ID NO: 1144), CAGguaaggg (SEQ ID NO: 1145), CAGguaaggu (SEQ ID NO: 1146), CAGguaagua (SEQ ID NO: 1147), CAGguaagug (SEQ ID NO: 1148), CAGguaaguu (SEQ ID NO: 1149), CAGguaauaa (SEQ ID NO: 1150), CAGguaauau (SEQ ID NO: 1151), CAGguaaucc (SEQ ID NO: 1152), CAGguaaugc (SEQ ID NO: 1153), CAGguaaugg (SEQ ID NO: 1154), CAGguaaugu (SEQ ID NO: 1155), CAGguaauua (SEQ ID NO: 1156), CAGguaauuc (SEQ ID NO: 1157), CAGguaauug (SEQ ID NO: 1158), CAGguaauuu (SEQ ID NO: 1159), CAGguacaaa (SEQ ID NO: 1160), CAGguacaag (SEQ ID NO: 1161), CAGguacaau (SEQ ID NO: 1162), CAGguacaca (SEQ ID NO: 1163), CAGguacacg (SEQ ID NO: 1164), CAGguacaga (SEQ ID NO: 1165), CAGguacagg (SEQ ID NO: 1166), CAGguacagu (SEQ ID NO: 1167), CAGguacaua (SEQ ID NO: 1168), CAGguacaug (SEQ ID NO: 1169), CAGguacauu (SEQ ID NO: 1170), CAGguaccac (SEQ ID NO: 1171), CAGguaccca (SEQ ID NO: 1172), CAGguacccg (SEQ ID NO: 1173), CAGguacccu (SEQ ID NO: 1174), CAGguaccgc (SEQ ID NO: 1175), CAGguaccgg (SEQ ID NO: 1176), CAGguaccuc (SEQ ID NO: 1177), CAGguaccug (SEQ ID NO: 1178), CAGguaccuu (SEQ ID NO: 1179), CAGguacgag (SEQ ID NO: 1180), CAGguacgca (SEQ ID NO: 1181), CAGguacgcc (SEQ ID NO: 1182), CAGguacggu (SEQ ID NO: 1183), CAGguacgua (SEQ ID NO: 1184), CAGguacgug (SEQ ID NO: 1185), CAGguacuaa (SEQ ID NO: 1186), CAGguacuag (SEQ ID NO: 1187), CAGguacuau (SEQ ID NO: 1188), CAGguacucc (SEQ ID NO: 1189), CAGguacucu (SEQ ID NO: 1190), CAGguacuga (SEQ ID NO: 1191), CAGguacugc (SEQ ID NO: 1192), CAGguacugu (SEQ ID NO: 1193), CAGguacuua (SEQ ID NO: 1194), CAGguacuuu (SEQ ID NO: 1195), CAGguagaaa (SEQ ID NO: 1196), CAGguagaac (SEQ ID NO: 1197), CAGguagaag (SEQ ID NO: 1198), CAGguagaca (SEQ ID NO: 1199), CAGguagacc (SEQ ID NO: 1200), CAGguagaga (SEQ ID NO: 1201), CAGguagauu (SEQ ID NO: 1202), CAGguagcaa (SEQ ID NO: 1203), CAGguagcac (SEQ ID NO: 1204), CAGguagcag (SEQ ID NO: 1205), CAGguagcca (SEQ ID NO: 1206), CAGguagcgu (SEQ ID NO: 1207), CAGguagcua (SEQ ID NO: 1208), CAGguagcuc (SEQ ID NO: 1209), CAGguagcug (SEQ ID NO: 1210), CAGguagcuu (SEQ ID NO: 1211), CAGguaggaa (SEQ ID NO: 1212), CAGguaggac (SEQ ID NO: 1213), CAGguaggag (SEQ ID NO: 1214), CAGguaggca (SEQ ID NO: 1215), CAGguaggga (SEQ ID NO: 1216), CAGguagggc (SEQ ID NO: 1217), CAGguagggg (SEQ ID NO: 1218), CAGguagggu (SEQ ID NO: 1219), CAGguaggua (SEQ ID NO: 1220), CAGguagguc (SEQ ID NO: 1221), CAGguaggug (SEQ ID NO: 1222), CAGguagguu (SEQ ID NO: 1223), CAGguaguaa (SEQ ID NO: 1224), CAGguaguau (SEQ ID NO: 1225), CAGguaguca (SEQ ID NO: 1226), CAGguagucc (SEQ ID NO: 1227), CAGguaguga (SEQ ID NO: 1228), CAGguagugu (SEQ ID NO: 1229), CAGguaguuc (SEQ ID NO: 1230), CAGguaguug (SEQ ID NO: 1231), CAGguaguuu (SEQ ID NO: 1232), CAGguauaag (SEQ ID NO: 1233), CAGguauaca (SEQ ID NO: 1234), CAGguauaga (SEQ ID NO: 1235), CAGguauauc (SEQ ID NO: 1236), CAGguauaug (SEQ ID NO: 1237), CAGguauauu (SEQ ID NO: 1238), CAGguaucag (SEQ ID NO: 1239), CAGguaucau (SEQ ID NO: 1240), CAGguauccu (SEQ ID NO: 1241), CAGguaucga (SEQ ID NO: 1242), CAGguaucgc (SEQ ID NO: 1243), CAGguaucua (SEQ ID NO: 1244), CAGguaucug (SEQ ID NO: 1245), CAGguaucuu (SEQ ID NO: 1246), CAGguaugaa (SEQ ID NO: 1247), CAGguaugac (SEQ ID NO: 1248), CAGguaugag (SEQ ID NO: 1249), CAGguaugau (SEQ ID NO: 1250), CAGguaugca (SEQ ID NO: 1251), CAGguaugcc (SEQ ID NO: 1252), CAGguaugcg (SEQ ID NO: 1253), CAGguaugcu (SEQ ID NO: 1254), CAGguaugga (SEQ ID NO: 1255), CAGguauggg (SEQ ID NO: 1256), CAGguauggu (SEQ ID NO: 1257), CAGguaugua (SEQ ID NO: 1258), CAGguauguc (SEQ ID NO: 1259), CAGguaugug (SEQ ID NO: 1260), CAGguauguu (SEQ ID NO: 1261), CAGguauuau (SEQ ID NO: 1262), CAGguauuca (SEQ ID NO: 1263), CAGguauucu (SEQ ID NO: 1264), CAGguauuga (SEQ ID NO: 1265), CAGguauugg (SEQ ID NO: 1266), CAGguauugu (SEQ ID NO: 1267), CAGguauuua (SEQ ID NO: 1268), CAGguauuuc (SEQ ID NO: 1269), CAGguauuug (SEQ ID NO: 1270), CAGguauuuu (SEQ ID NO: 1271), CAGgucaaca (SEQ ID NO: 1272), CAGgucaaug (SEQ ID NO: 1273), CAGgucacgu (SEQ ID NO: 1274), CAGgucagaa (SEQ ID NO: 1275), CAGgucagac (SEQ ID NO: 1276), CAGgucagca (SEQ ID NO: 1277), CAGgucagcc (SEQ ID NO: 1278), CAGgucagcg (SEQ ID NO: 1279), CAGgucagga (SEQ ID NO: 1280), CAGgucagua (SEQ ID NO: 1281), CAGgucaguc (SEQ ID NO: 1282), CAGgucagug (SEQ ID NO: 1283), CAGgucaguu (SEQ ID NO: 1284), CAGgucaucc (SEQ ID NO: 1285), CAGgucaugc (SEQ ID NO: 1286), CAGgucauua (SEQ ID NO: 1287), CAGgucauuu (SEQ ID NO: 1288), CAGguccacc (SEQ ID NO: 1289), CAGguccacu (SEQ ID NO: 1290), CAGguccagu (SEQ ID NO: 1291), CAGguccauc (SEQ ID NO: 1292), CAGguccauu (SEQ ID NO: 1293), CAGgucccag (SEQ ID NO: 1294), CAGgucccug (SEQ ID NO: 1295), CAGguccuga (SEQ ID NO: 1296), CAGguccugc (SEQ ID NO: 1297), CAGguccugg (SEQ ID NO: 1298), CAGgucggcc (SEQ ID NO: 1299), CAGgucggug (SEQ ID NO: 1300), CAGgucguug (SEQ ID NO: 1301), CAGgucucuc (SEQ ID NO: 1302), CAGgucucuu (SEQ ID NO: 1303), CAGgucugag (SEQ ID NO: 1304), CAGgucugcc (SEQ ID NO: 1305), CAGgucugcg (SEQ ID NO: 1306), CAGgucugga (SEQ ID NO: 1307), CAGgucuggu (SEQ ID NO: 1308), CAGgucugua (SEQ ID NO: 1309), CAGgucuguc (SEQ ID NO: 1310), CAGgucugug (SEQ ID NO: 1311), CAGgucuguu (SEQ ID NO: 1312), CAGgucuucc (SEQ ID NO: 1313), CAGgucuuuc (SEQ ID NO: 1314), CAGgugaaag (SEQ ID NO: 1315), CAGgugaaau (SEQ ID NO: 1316), CAGgugaaca (SEQ ID NO: 1317), CAGgugaaga (SEQ ID NO: 1318), CAGgugaagg (SEQ ID NO: 1319), CAGgugaaua (SEQ ID NO: 1320), CAGgugaauc (SEQ ID NO: 1321), CAGgugaauu (SEQ ID NO: 1322), CAGgugacaa (SEQ ID NO: 1323), CAGgugacau (SEQ ID NO: 1324), CAGgugacca (SEQ ID NO: 1325), CAGgugaccc (SEQ ID NO: 1326), CAGgugaccg (SEQ ID NO: 1327), CAGgugaccu (SEQ ID NO: 1328), CAGgugacgg (SEQ ID NO: 1329), CAGgugacua (SEQ ID NO: 1330), CAGgugacuc (SEQ ID NO: 1331), CAGgugacug (SEQ ID NO: 1332), CAGgugagaa (SEQ ID NO: 1333), CAGgugagac (SEQ ID NO: 1334), CAGgugagag (SEQ ID NO: 1335), CAGgugagau (SEQ ID NO: 1336), CAGgugagca (SEQ ID NO: 1337), CAGgugagcc (SEQ ID NO: 1338), CAGgugagcg (SEQ ID NO: 1339), CAGgugagcu (SEQ ID NO: 1340), CAGgugagga (SEQ ID NO: 1341), CAGgugaggc (SEQ ID NO: 1342), CAGgugaggg (SEQ ID NO: 1343), CAGgugaggu (SEQ ID NO: 1344), CAGgugagua (SEQ ID NO: 1345), CAGgugaguc (SEQ ID NO: 1346), CAGgugagug (SEQ ID NO: 1347), CAGgugaguu (SEQ ID NO: 1348), CAGgugauaa (SEQ ID NO: 1349), CAGgugaucc (SEQ ID NO: 1350), CAGgugaucu (SEQ ID NO: 1351), CAGgugaugc (SEQ ID NO: 1352), CAGgugaugg (SEQ ID NO: 1353), CAGgugaugu (SEQ ID NO: 1354), CAGgugauua (SEQ ID NO: 1355), CAGgugauuc (SEQ ID NO: 1356), CAGgugauug (SEQ ID NO: 1357), CAGgugauuu (SEQ ID NO: 1358), CAGgugcaaa (SEQ ID NO: 1359), CAGgugcaag (SEQ ID NO: 1360), CAGgugcaca (SEQ ID NO: 1361), CAGgugcacg (SEQ ID NO: 1362), CAGgugcaga (SEQ ID NO: 1363), CAGgugcagg (SEQ ID NO: 1364), CAGgugcaua (SEQ ID NO: 1365), CAGgugcauc (SEQ ID NO: 1366), CAGgugcaug (SEQ ID NO: 1367), CAGgugccaa (SEQ ID NO: 1368), CAGgugccca (SEQ ID NO: 1369), CAGgugcccc (SEQ ID NO: 1370), CAGgugcccg (SEQ ID NO: 1371), CAGgugccua (SEQ ID NO: 1372), CAGgugccug (SEQ ID NO: 1373), CAGgugcgaa (SEQ ID NO: 1374), CAGgugcgca (SEQ ID NO: 1375), CAGgugcgcc (SEQ ID NO: 1376), CAGgugcgcg (SEQ ID NO: 1377), CAGgugcgga (SEQ ID NO: 1378), CAGgugcggu (SEQ ID NO: 1379), CAGgugcgua (SEQ ID NO: 1380), CAGgugcguc (SEQ ID NO: 1381), CAGgugcgug (SEQ ID NO: 1382), CAGgugcuag (SEQ ID NO: 1383), CAGgugcuau (SEQ ID NO: 1384), CAGgugcuca (SEQ ID NO: 1385), CAGgugcucc (SEQ ID NO: 1386), CAGgugcucg (SEQ ID NO: 1387), CAGgugcugc (SEQ ID NO: 1388), CAGgugcugg (SEQ ID NO: 1389), CAGgugcuua (SEQ ID NO: 1390), CAGgugcuuc (SEQ ID NO: 1391), CAGgugcuug (SEQ ID NO: 1392), CAGguggaac (SEQ ID NO: 1393), CAGguggaag (SEQ ID NO: 1394), CAGguggaau (SEQ ID NO: 1395), CAGguggaga (SEQ ID NO: 1396), CAGguggagu (SEQ ID NO: 1397), CAGguggauu (SEQ ID NO: 1398), CAGguggcca (SEQ ID NO: 1399), CAGguggcuc (SEQ ID NO: 1400), CAGguggcug (SEQ ID NO: 1401), CAGgugggaa (SEQ ID NO: 1402), CAGgugggac (SEQ ID NO: 1403), CAGgugggag (SEQ ID NO: 1404), CAGgugggau (SEQ ID NO: 1405), CAGgugggca (SEQ ID NO: 1406), CAGgugggcc (SEQ ID NO: 1407), CAGgugggcu (SEQ ID NO: 1408), CAGgugggga (SEQ ID NO: 1409), CAGguggggc (SEQ ID NO: 1410), CAGguggggg (SEQ ID NO: 1411), CAGguggggu (SEQ ID NO: 1412), CAGgugggua (SEQ ID NO: 1413), CAGguggguc (SEQ ID NO: 1414), CAGgugggug (SEQ ID NO: 1415), CAGguggguu (SEQ ID NO: 1416), CAGguggucu (SEQ ID NO: 1417), CAGguggugg (SEQ ID NO: 1418), CAGgugguug (SEQ ID NO: 1419), CAGguguaca (SEQ ID NO: 1420), CAGguguagg (SEQ ID NO: 1421), CAGguguauc (SEQ ID NO: 1422), CAGgugucac (SEQ ID NO: 1423), CAGgugucag (SEQ ID NO: 1424), CAGgugucca (SEQ ID NO: 1425), CAGguguccu (SEQ ID NO: 1426), CAGgugucua (SEQ ID NO: 1427), CAGgugucuc (SEQ ID NO: 1428), CAGgugucug (SEQ ID NO: 1429), CAGgugugaa (SEQ ID NO: 1430), CAGgugugac (SEQ ID NO: 1431), CAGgugugag (SEQ ID NO: 1432), CAGgugugau (SEQ ID NO: 1433), CAGgugugca (SEQ ID NO: 1434), CAGgugugcc (SEQ ID NO: 1435), CAGgugugcg (SEQ ID NO: 1436), CAGgugugcu (SEQ ID NO: 1437), CAGgugugga (SEQ ID NO: 1438), CAGguguggc (SEQ ID NO: 1439), CAGgugugua (SEQ ID NO: 1440), CAGguguguc (SEQ ID NO: 1441), CAGgugugug (SEQ ID NO: 1442), CAGguguguu (SEQ ID NO: 1443), CAGguguuua (SEQ ID NO: 1444), CAGguuaaaa (SEQ ID NO: 1445), CAGguuaaua (SEQ ID NO: 1446), CAGguuaauc (SEQ ID NO: 1447), CAGguuaccu (SEQ ID NO: 1448), CAGguuagaa (SEQ ID NO: 1449), CAGguuagag (SEQ ID NO: 1450), CAGguuagau (SEQ ID NO: 1451), CAGguuagcc (SEQ ID NO: 1452), CAGguuaggg (SEQ ID NO: 1453), CAGguuaggu (SEQ ID NO: 1454), CAGguuagua (SEQ ID NO: 1455), CAGguuaguc (SEQ ID NO: 1456), CAGguuagug (SEQ ID NO: 1457), CAGguuaguu (SEQ ID NO: 1458), CAGguuauca (SEQ ID NO: 1459), CAGguuaugu (SEQ ID NO: 1460), CAGguuauua (SEQ ID NO: 1461), CAGguuauug (SEQ ID NO: 1462), CAGguucaaa (SEQ ID NO: 1463), CAGguucaac (SEQ ID NO: 1464), CAGguucaag (SEQ ID NO: 1465), CAGguucaca (SEQ ID NO: 1466), CAGguucacg (SEQ ID NO: 1467), CAGguucagg (SEQ ID NO: 1468), CAGguucaug (SEQ ID NO: 1469), CAGguuccag (SEQ ID NO: 1470), CAGguuccca (SEQ ID NO: 1471), CAGguucccg (SEQ ID NO: 1472), CAGguucgaa (SEQ ID NO: 1473), CAGguucgag (SEQ ID NO: 1474), CAGguucuau (SEQ ID NO: 1475), CAGguucugc (SEQ ID NO: 1476), CAGguucuua (SEQ ID NO: 1477), CAGguucuuc (SEQ ID NO: 1478), CAGguucuuu (SEQ ID NO: 1479), CAGguugaac (SEQ ID NO: 1480), CAGguugaag (SEQ ID NO: 1481), CAGguugagu (SEQ ID NO: 1482), CAGguugaua (SEQ ID NO: 1483), CAGguuggag (SEQ ID NO: 1484), CAGguuggca (SEQ ID NO: 1485), CAGguuggcc (SEQ ID NO: 1486), CAGguugguc (SEQ ID NO: 1487), CAGguuggug (SEQ ID NO: 1488), CAGguugguu (SEQ ID NO: 1489), CAGguuguaa (SEQ ID NO: 1490), CAGguuguac (SEQ ID NO: 1491), CAGguuguau (SEQ ID NO: 1492), CAGguuguca (SEQ ID NO: 1493), CAGguuguga (SEQ ID NO: 1494), CAGguuguug (SEQ ID NO: 1495), CAGguuuaag (SEQ ID NO: 1496), CAGguuuacc (SEQ ID NO: 1497), CAGguuuagc (SEQ ID NO: 1498), CAGguuuagu (SEQ ID NO: 1499), CAGguuucuu (SEQ ID NO: 1500), CAGguuugaa (SEQ ID NO: 1501), CAGguuugag (SEQ ID NO: 1502), CAGguuugau (SEQ ID NO: 1503), CAGguuugcc (SEQ ID NO: 1504), CAGguuugcu (SEQ ID NO: 1505), CAGguuuggg (SEQ ID NO: 1506), CAGguuuggu (SEQ ID NO: 1507), CAGguuugua (SEQ ID NO: 1508), CAGguuugug (SEQ ID NO: 1509), CAGguuuguu (SEQ ID NO: 1510), CAGguuuucu (SEQ ID NO: 1511), CAGguuuugg (SEQ ID NO: 1512), CAGguuuuuc (SEQ ID NO: 1513), CAGguuuuuu (SEQ ID NO: 1514), CAUgcagguu (SEQ ID NO: 1515), CAUguaaaac (SEQ ID NO: 1516), CAUguaacua (SEQ ID NO: 1517), CAUguaagaa (SEQ ID NO: 1518), CAUguaagag (SEQ ID NO: 1519), CAUguaagau (SEQ ID NO: 1520), CAUguaagcc (SEQ ID NO: 1521), CAUguaagua (SEQ ID NO: 1522), CAUguaagug (SEQ ID NO: 1523), CAUguaaguu (SEQ ID NO: 1524), CAUguaauua (SEQ ID NO: 1525), CAUguacaua (SEQ ID NO: 1526), CAUguaccac (SEQ ID NO: 1527), CAUguacguu (SEQ ID NO: 1528), CAUguaggua (SEQ ID NO: 1529), CAUguaggug (SEQ ID NO: 1530), CAUguagguu (SEQ ID NO: 1531), CAUguaugaa (SEQ ID NO: 1532), CAUguaugua (SEQ ID NO: 1533), CAUguaugug (SEQ ID NO: 1534), CAUguauguu (SEQ ID NO: 1535), CAUgugagaa (SEQ ID NO: 1536), CAUgugagca (SEQ ID NO: 1537), CAUgugagcu (SEQ ID NO: 1538), CAUgugagua (SEQ ID NO: 1539), CAUgugaguc (SEQ ID NO: 1540), CAUgugagug (SEQ ID NO: 1541), CAUgugaguu (SEQ ID NO: 1542), CAUgugcgua (SEQ ID NO: 1543), CAUgugggaa (SEQ ID NO: 1544), CAUguggguu (SEQ ID NO: 1545), CAUgugugug (SEQ ID NO: 1546), CAUguguguu (SEQ ID NO: 1547), CAUguuaaua (SEQ ID NO: 1548), CAUguuagcc (SEQ ID NO: 1549), CCAguaagau (SEQ ID NO: 1550), CCAguaagca (SEQ ID NO: 1551), CCAguaagcc (SEQ ID NO: 1552), CCAguaagcu (SEQ ID NO: 1553), CCAguaagga (SEQ ID NO: 1554), CCAguaagua (SEQ ID NO: 1555), CCAguaaguc (SEQ ID NO: 1556), CCAguaagug (SEQ ID NO: 1557), CCAguaaguu (SEQ ID NO: 1558), CCAguaauug (SEQ ID NO: 1559), CCAguacggg (SEQ ID NO: 1560), CCAguagguc (SEQ ID NO: 1561), CCAguauugu (SEQ ID NO: 1562), CCAgugaggc (SEQ ID NO: 1563), CCAgugagua (SEQ ID NO: 1564), CCAgugagug (SEQ ID NO: 1565), CCAguggguc (SEQ ID NO: 1566), CCAguuaguu (SEQ ID NO: 1567), CCAguugagu (SEQ ID NO: 1568), CCCguaagau (SEQ ID NO: 1569), CCCguauguc (SEQ ID NO: 1570), CCCguauguu (SEQ ID NO: 1571), CCCguccugc (SEQ ID NO: 1572), CCCgugagug (SEQ ID NO: 1573), CCGguaaaga (SEQ ID NO: 1574), CCGguaagau (SEQ ID NO: 1575), CCGguaagcc (SEQ ID NO: 1576), CCGguaagga (SEQ ID NO: 1577), CCGguaaggc (SEQ ID NO: 1578), CCGguaaugg (SEQ ID NO: 1579), CCGguacagu (SEQ ID NO: 1580), CCGguacuga (SEQ ID NO: 1581), CCGguauucc (SEQ ID NO: 1582), CCGgucagug (SEQ ID NO: 1583), CCGgugaaaa (SEQ ID NO: 1584), CCGgugagaa (SEQ ID NO: 1585), CCGgugaggg (SEQ ID NO: 1586), CCGgugagug (SEQ ID NO: 1587), CCGgugaguu (SEQ ID NO: 1588), CCGgugcgcg (SEQ ID NO: 1589), CCGgugggcg (SEQ ID NO: 1590), CCGguugguc (SEQ ID NO: 1591), CCUguaaaug (SEQ ID NO: 1592), CCUguaaauu (SEQ ID NO: 1593), CCUguaagaa (SEQ ID NO: 1594), CCUguaagac (SEQ ID NO: 1595), CCUguaagag (SEQ ID NO: 1596), CCUguaagca (SEQ ID NO: 1597), CCUguaagcg (SEQ ID NO: 1598), CCUguaagga (SEQ ID NO: 1599), CCUguaaguu (SEQ ID NO: 1600), CCUguaggua (SEQ ID NO: 1601), CCUguaggug (SEQ ID NO: 1602), CCUguaucuu (SEQ ID NO: 1603), CCUguauggu (SEQ ID NO: 1604), CCUguaugug (SEQ ID NO: 1605), CCUgugagaa (SEQ ID NO: 1606), CCUgugagca (SEQ ID NO: 1607), CCUgugaggg (SEQ ID NO: 1608), CCUgugaguc (SEQ ID NO: 1609), CCUgugagug (SEQ ID NO: 1610), CCUgugaguu (SEQ ID NO: 1611), CCUguggcuc (SEQ ID NO: 1612), CCUgugggua (SEQ ID NO: 1613), CCUgugugua (SEQ ID NO: 1614), CCUguuagaa (SEQ ID NO: 1615), CGAguaaggg (SEQ ID NO: 1616), CGAguaaggu (SEQ ID NO: 1617), CGAguagcug (SEQ ID NO: 1618), CGAguaggug (SEQ ID NO: 1619), CGAguagguu (SEQ ID NO: 1620), CGAgugagca (SEQ ID NO: 1621), CGCguaagag (SEQ ID NO: 1622), CGGgcaggca (SEQ ID NO: 1623), CGGguaagcc (SEQ ID NO: 1624), CGGguaagcu (SEQ ID NO: 1625), CGGguaaguu (SEQ ID NO: 1626), CGGguaauuc (SEQ ID NO: 1627), CGGguaauuu (SEQ ID NO: 1628), CGGguacagu (SEQ ID NO: 1629), CGGguacggg (SEQ ID NO: 1630), CGGguaggag (SEQ ID NO: 1631), CGGguaggcc (SEQ ID NO: 1632), CGGguaggug (SEQ ID NO: 1633), CGGguauuua (SEQ ID NO: 1634), CGGgucugag (SEQ ID NO: 1635), CGGgugaccg (SEQ ID NO: 1636), CGGgugacuc (SEQ ID NO: 1637), CGGgugagaa (SEQ ID NO: 1638), CGGgugaggg (SEQ ID NO: 1639), CGGgugaggu (SEQ ID NO: 1640), CGGgugagua (SEQ ID NO: 1641), CGGgugagug (SEQ ID NO: 1642), CGGgugaguu (SEQ ID NO: 1643), CGGgugauuu (SEQ ID NO: 1644), CGGgugccuu (SEQ ID NO: 1645), CGGgugggag (SEQ ID NO: 1646), CGGgugggug (SEQ ID NO: 1647), CGGguggguu (SEQ ID NO: 1648), CGGguguguc (SEQ ID NO: 1649), CGGgugugug (SEQ ID NO: 1650), CGGguguguu (SEQ ID NO: 1651), CGGguucaag (SEQ ID NO: 1652), CGGguucaug (SEQ ID NO: 1653), CGGguuugcu (SEQ ID NO: 1654), CGUguagggu (SEQ ID NO: 1655), CGUguaugca (SEQ ID NO: 1656), CGUguaugua (SEQ ID NO: 1657), CGUgucugua (SEQ ID NO: 1658), CGUgugagug (SEQ ID NO: 1659), CGUguuuucu (SEQ ID NO: 1660), CUAguaaaug (SEQ ID NO: 1661), CUAguaagcg (SEQ ID NO: 1662), CUAguaagcu (SEQ ID NO: 1663), CUAguaagua (SEQ ID NO: 1664), CUAguaaguc (SEQ ID NO: 1665), CUAguaagug (SEQ ID NO: 1666), CUAguaaguu (SEQ ID NO: 1667), CUAguaauuu (SEQ ID NO: 1668), CUAguaggua (SEQ ID NO: 1669), CUAguagguu (SEQ ID NO: 1670), CUAguaugua (SEQ ID NO: 1671), CUAguauguu (SEQ ID NO: 1672), CUAgugagua (SEQ ID NO: 1673), CUCguaagca (SEQ ID NO: 1674), CUCguaagug (SEQ ID NO: 1675), CUCguaaguu (SEQ ID NO: 1676), CUCguaucug (SEQ ID NO: 1677), CUCgucugug (SEQ ID NO: 1678), CUCgugaaua (SEQ ID NO: 1679), CUCgugagua (SEQ ID NO: 1680), CUCgugauua (SEQ ID NO: 1681), CUGguaaaaa (SEQ ID NO: 1682), CUGguaaaau (SEQ ID NO: 1683), CUGguaaacc (SEQ ID NO: 1684), CUGguaaacg (SEQ ID NO: 1685), CUGguaaagc (SEQ ID NO: 1686), CUGguaaaua (SEQ ID NO: 1687), CUGguaaauc (SEQ ID NO: 1688), CUGguaaaug (SEQ ID NO: 1689), CUGguaaauu (SEQ ID NO: 1690), CUGguaacac (SEQ ID NO: 1691), CUGguaacag (SEQ ID NO: 1692), CUGguaaccc (SEQ ID NO: 1693), CUGguaaccg (SEQ ID NO: 1694), CUGguaacug (SEQ ID NO: 1695), CUGguaacuu (SEQ ID NO: 1696), CUGguaagaa (SEQ ID NO: 1697), CUGguaagag (SEQ ID NO: 1698), CUGguaagau (SEQ ID NO: 1699), CUGguaagca (SEQ ID NO: 1700), CUGguaagcc (SEQ ID NO: 1701), CUGguaagcu (SEQ ID NO: 1702), CUGguaagga (SEQ ID NO: 1703), CUGguaaggc (SEQ ID NO: 1704), CUGguaaggg (SEQ ID NO: 1705), CUGguaaggu (SEQ ID NO: 1706), CUGguaagua (SEQ ID NO: 1707), CUGguaagug (SEQ ID NO: 1708), CUGguaaguu (SEQ ID NO: 1709), CUGguaauga (SEQ ID NO: 1710), CUGguaaugc (SEQ ID NO: 1711), CUGguaauuc (SEQ ID NO: 1712), CUGguaauuu (SEQ ID NO: 1713), CUGguacaac (SEQ ID NO: 1714), CUGguacaau (SEQ ID NO: 1715), CUGguacaga (SEQ ID NO: 1716), CUGguacaua (SEQ ID NO: 1717), CUGguacauu (SEQ ID NO: 1718), CUGguaccau (SEQ ID NO: 1719), CUGguacguu (SEQ ID NO: 1720), CUGguacuaa (SEQ ID NO: 1721), CUGguacuug (SEQ ID NO: 1722), CUGguacuuu (SEQ ID NO: 1723), CUGguagaga (SEQ ID NO: 1724), CUGguagaua (SEQ ID NO: 1725), CUGguagcgu (SEQ ID NO: 1726), CUGguaggau (SEQ ID NO: 1727), CUGguaggca (SEQ ID NO: 1728), CUGguaggua (SEQ ID NO: 1729), CUGguagguc (SEQ ID NO: 1730), CUGguaggug (SEQ ID NO: 1731), CUGguaucaa (SEQ ID NO: 1732), CUGguaugau (SEQ ID NO: 1733), CUGguauggc (SEQ ID NO: 1734), CUGguauggu (SEQ ID NO: 1735), CUGguaugua (SEQ ID NO: 1736), CUGguaugug (SEQ ID NO: 1737), CUGguauguu (SEQ ID NO: 1738), CUGguauuga (SEQ ID NO: 1739), CUGguauuuc (SEQ ID NO: 1740), CUGguauuuu (SEQ ID NO: 1741), CUGgucaaca (SEQ ID NO: 1742), CUGgucagag (SEQ ID NO: 1743), CUGgucccgc (SEQ ID NO: 1744), CUGgucggua (SEQ ID NO: 1745), CUGgucuggg (SEQ ID NO: 1746), CUGgugaagu (SEQ ID NO: 1747), CUGgugaaua (SEQ ID NO: 1748), CUGgugaauu (SEQ ID NO: 1749), CUGgugacua (SEQ ID NO: 1750), CUGgugagaa (SEQ ID NO: 1751), CUGgugagac (SEQ ID NO: 1752), CUGgugagca (SEQ ID NO: 1753), CUGgugagcu (SEQ ID NO: 1754), CUGgugagga (SEQ ID NO: 1755), CUGgugaggc (SEQ ID NO: 1756), CUGgugaggg (SEQ ID NO: 1757), CUGgugaggu (SEQ ID NO: 1758), CUGgugagua (SEQ ID NO: 1759), CUGgugaguc (SEQ ID NO: 1760), CUGgugagug (SEQ ID NO: 1761), CUGgugaguu (SEQ ID NO: 1762), CUGgugauua (SEQ ID NO: 1763), CUGgugauuu (SEQ ID NO: 1764), CUGgugcaga (SEQ ID NO: 1765), CUGgugcgcu (SEQ ID NO: 1766), CUGgugcgug (SEQ ID NO: 1767), CUGgugcuga (SEQ ID NO: 1768), CUGgugggag (SEQ ID NO: 1769), CUGgugggga (SEQ ID NO: 1770), CUGgugggua (SEQ ID NO: 1771), CUGguggguc (SEQ ID NO: 1772), CUGgugggug (SEQ ID NO: 1773), CUGguggguu (SEQ ID NO: 1774), CUGgugugaa (SEQ ID NO: 1775), CUGgugugca (SEQ ID NO: 1776), CUGgugugcu (SEQ ID NO: 1777), CUGguguggu (SEQ ID NO: 1778), CUGgugugug (SEQ ID NO: 1779), CUGguguguu (SEQ ID NO: 1780), CUGguuagcu (SEQ ID NO: 1781), CUGguuagug (SEQ ID NO: 1782), CUGguucgug (SEQ ID NO: 1783), CUGguuggcu (SEQ ID NO: 1784), CUGguuguuu (SEQ ID NO: 1785), CUGguuugua (SEQ ID NO: 1786), CUGguuuguc (SEQ ID NO: 1787), CUGguuugug (SEQ ID NO: 1788), CUUguaaaug (SEQ ID NO: 1789), CUUguaagcu (SEQ ID NO: 1790), CUUguaagga (SEQ ID NO: 1791), CUUguaaggc (SEQ ID NO: 1792), CUUguaagua (SEQ ID NO: 1793), CUUguaagug (SEQ ID NO: 1794), CUUguaaguu (SEQ ID NO: 1795), CUUguacguc (SEQ ID NO: 1796), CUUguacgug (SEQ ID NO: 1797), CUUguaggua (SEQ ID NO: 1798), CUUguagugc (SEQ ID NO: 1799), CUUguauagg (SEQ ID NO: 1800), CUUgucagua (SEQ ID NO: 1801), CUUgugagua (SEQ ID NO: 1802), CUUgugaguc (SEQ ID NO: 1803), CUUgugaguu (SEQ ID NO: 1804), CUUguggguu (SEQ ID NO: 1805), CUUgugugua (SEQ ID NO: 1806), CUUguuagug (SEQ ID NO: 1807), CUUguuugag (SEQ ID NO: 1808), GAAguaaaac (SEQ ID NO: 1809), GAAguaaagc (SEQ ID NO: 1810), GAAguaaagu (SEQ ID NO: 1811), GAAguaaaua (SEQ ID NO: 1812), GAAguaaauu (SEQ ID NO: 1813), GAAguaagaa (SEQ ID NO: 1814), GAAguaagcc (SEQ ID NO: 1815), GAAguaagcu (SEQ ID NO: 1816), GAAguaagga (SEQ ID NO: 1817), GAAguaagua (SEQ ID NO: 1818), GAAguaagug (SEQ ID NO: 1819), GAAguaaguu (SEQ ID NO: 1820), GAAguaauau (SEQ ID NO: 1821), GAAguaaugc (SEQ ID NO: 1822), GAAguaauua (SEQ ID NO: 1823), GAAguaauuu (SEQ ID NO: 1824), GAAguaccau (SEQ ID NO: 1825), GAAguacgua (SEQ ID NO: 1826), GAAguacguc (SEQ ID NO: 1827), GAAguaggca (SEQ ID NO: 1828), GAAguagguc (SEQ ID NO: 1829), GAAguauaaa (SEQ ID NO: 1830), GAAguaugcu (SEQ ID NO: 1831), GAAguaugug (SEQ ID NO: 1832), GAAguauguu (SEQ ID NO: 1833), GAAguauuaa (SEQ ID NO: 1834), GAAgucagug (SEQ ID NO: 1835), GAAgugagag (SEQ ID NO: 1836), GAAgugagcg (SEQ ID NO: 1837), GAAgugaggu (SEQ ID NO: 1838), GAAgugaguc (SEQ ID NO: 1839), GAAgugagug (SEQ ID NO: 1840), GAAgugaguu (SEQ ID NO: 1841), GAAgugauaa (SEQ ID NO: 1842), GAAgugauuc (SEQ ID NO: 1843), GAAgugcgug (SEQ ID NO: 1844), GAAguguggg (SEQ ID NO: 1845), GAAguguguc (SEQ ID NO: 1846), GAAguuggug (SEQ ID NO: 1847), GACguaaagu (SEQ ID NO: 1848), GACguaagcu (SEQ ID NO: 1849), GACguaagua (SEQ ID NO: 1850), GACguaaugg (SEQ ID NO: 1851), GACguaugcc (SEQ ID NO: 1852), GACguauguu (SEQ ID NO: 1853), GACgugagcc (SEQ ID NO: 1854), GACgugagug (SEQ ID NO: 1855), GAGgcaaaug (SEQ ID NO: 1856), GAGgcaagag (SEQ ID NO: 1857), GAGgcaagua (SEQ ID NO: 1858), GAGgcaagug (SEQ ID NO: 1859), GAGgcaaguu (SEQ ID NO: 1860), GAGgcacgag (SEQ ID NO: 1861), GAGgcaggga (SEQ ID NO: 1862), GAGgcaugug (SEQ ID NO: 1863), GAGgcgaagg (SEQ ID NO: 1864), GAGguaaaaa (SEQ ID NO: 1865), GAGguaaaac (SEQ ID NO: 1866), GAGguaaaag (SEQ ID NO: 1867), GAGguaaaau (SEQ ID NO: 1868), GAGguaaacc (SEQ ID NO: 1869), GAGguaaaga (SEQ ID NO: 1870), GAGguaaagc (SEQ ID NO: 1871), GAGguaaagu (SEQ ID NO: 1872), GAGguaaaua (SEQ ID NO: 1873), GAGguaaauc (SEQ ID NO: 1874), GAGguaaaug (SEQ ID NO: 1875), GAGguaaauu (SEQ ID NO: 1876), GAGguaacaa (SEQ ID NO: 1877), GAGguaacag (SEQ ID NO: 1878), GAGguaacca (SEQ ID NO: 1879), GAGguaaccu (SEQ ID NO: 1880), GAGguaacuu (SEQ ID NO: 1881), GAGguaagaa (SEQ ID NO: 1882), GAGguaagag (SEQ ID NO: 1883), GAGguaagau (SEQ ID NO: 1884), GAGguaagca (SEQ ID NO: 1885), GAGguaagcc (SEQ ID NO: 1886), GAGguaagcg (SEQ ID NO: 1887), GAGguaagcu (SEQ ID NO: 1888), GAGguaagga (SEQ ID NO: 1889), GAGguaaggc (SEQ ID NO: 1890), GAGguaaggg (SEQ ID NO: 1891), GAGguaaggu (SEQ ID NO: 1892), GAGguaagua (SEQ ID NO: 1893), GAGguaaguc (SEQ ID NO: 1894), GAGguaauaa (SEQ ID NO: 1895), GAGguaauac (SEQ ID NO: 1896), GAGguaauau (SEQ ID NO: 1897), GAGguaauca (SEQ ID NO: 1898), GAGguaaucu (SEQ ID NO: 1899), GAGguaaugg (SEQ ID NO: 1900), GAGguaaugu (SEQ ID NO: 1901), GAGguaauug (SEQ ID NO: 1902), GAGguaauuu (SEQ ID NO: 1903), GAGguacaaa (SEQ ID NO: 1904), GAGguacaac (SEQ ID NO: 1905), GAGguacaga (SEQ ID NO: 1906), GAGguacagc (SEQ ID NO: 1907), GAGguacagu (SEQ ID NO: 1908), GAGguacaua (SEQ ID NO: 1909), GAGguacauu (SEQ ID NO: 1910), GAGguaccag (SEQ ID NO: 1911), GAGguaccga (SEQ ID NO: 1912), GAGguaccug (SEQ ID NO: 1913), GAGguaccuu (SEQ ID NO: 1914), GAGguacuag (SEQ ID NO: 1915), GAGguacuau (SEQ ID NO: 1916), GAGguacucc (SEQ ID NO: 1917), GAGguacugc (SEQ ID NO: 1918), GAGguacugg (SEQ ID NO: 1919), GAGguacugu (SEQ ID NO: 1920), GAGguacuug (SEQ ID NO: 1921), GAGguacuuu (SEQ ID NO: 1922), GAGguagaag (SEQ ID NO: 1923), GAGguagaga (SEQ ID NO: 1924), GAGguagagg (SEQ ID NO: 1925), GAGguagagu (SEQ ID NO: 1926), GAGguagauc (SEQ ID NO: 1927), GAGguagcua (SEQ ID NO: 1928), GAGguagcug (SEQ ID NO: 1929), GAGguaggaa (SEQ ID NO: 1930), GAGguaggag (SEQ ID NO: 1931), GAGguaggca (SEQ ID NO: 1932), GAGguaggcu (SEQ ID NO: 1933), GAGguaggga (SEQ ID NO: 1934), GAGguagggc (SEQ ID NO: 1935), GAGguagggg (SEQ ID NO: 1936), GAGguaggua (SEQ ID NO: 1937), GAGguaggug (SEQ ID NO: 1938), GAGguagguu (SEQ ID NO: 1939), GAGguaguaa (SEQ ID NO: 1940), GAGguaguag (SEQ ID NO: 1941), GAGguaguau (SEQ ID NO: 1942), GAGguagucu (SEQ ID NO: 1943), GAGguagugc (SEQ ID NO: 1944), GAGguagugg (SEQ ID NO: 1945), GAGguaguua (SEQ ID NO: 1946), GAGguaguug (SEQ ID NO: 1947), GAGguauaag (SEQ ID NO: 1948), GAGguauacu (SEQ ID NO: 1949), GAGguauagc (SEQ ID NO: 1950), GAGguauaug (SEQ ID NO: 1951), GAGguauauu (SEQ ID NO: 1952), GAGguaucau (SEQ ID NO: 1953), GAGguaucug (SEQ ID NO: 1954), GAGguaucuu (SEQ ID NO: 1955), GAGguaugaa (SEQ ID NO: 1956), GAGguaugac (SEQ ID NO: 1957), GAGguaugag (SEQ ID NO: 1958), GAGguaugcc (SEQ ID NO: 1959), GAGguaugcg (SEQ ID NO: 1960), GAGguaugcu (SEQ ID NO: 1961), GAGguaugga (SEQ ID NO: 1962), GAGguauggg (SEQ ID NO: 1963), GAGguauggu (SEQ ID NO: 1964), GAGguaugua (SEQ ID NO: 1965), GAGguauguc (SEQ ID NO: 1966), GAGguaugug (SEQ ID NO: 1967), GAGguauguu (SEQ ID NO: 1968), GAGguauucc (SEQ ID NO: 1969), GAGguauuga (SEQ ID NO: 1970), GAGguauugu (SEQ ID NO: 1971), GAGguauuua (SEQ ID NO: 1972), GAGguauuuc (SEQ ID NO: 1973), GAGguauuug (SEQ ID NO: 1974), GAGguauuuu (SEQ ID NO: 1975), GAGgucaaca (SEQ ID NO: 1976), GAGgucaagg (SEQ ID NO: 1977), GAGgucaaug (SEQ ID NO: 1978), GAGgucacug (SEQ ID NO: 1979), GAGgucagaa (SEQ ID NO: 1980), GAGgucagag (SEQ ID NO: 1981), GAGgucagcu (SEQ ID NO: 1982), GAGgucagga (SEQ ID NO: 1983), GAGgucaggc (SEQ ID NO: 1984), GAGgucaggg (SEQ ID NO: 1985), GAGgucaggu (SEQ ID NO: 1986), GAGgucagua (SEQ ID NO: 1987), GAGgucauau (SEQ ID NO: 1988), GAGgucaugu (SEQ ID NO: 1989), GAGgucauuu (SEQ ID NO: 1990), GAGguccaua (SEQ ID NO: 1991), GAGguccauc (SEQ ID NO: 1992), GAGguccggg (SEQ ID NO: 1993), GAGguccggu (SEQ ID NO: 1994), GAGguccuug (SEQ ID NO: 1995), GAGgucgggg (SEQ ID NO: 1996), GAGgucucgu (SEQ ID NO: 1997), GAGgucugag (SEQ ID NO: 1998), GAGgucuggu (SEQ ID NO: 1999), GAGgucuguc (SEQ ID NO: 2000), GAGgucuguu (SEQ ID NO: 2001), GAGgucuuuu (SEQ ID NO: 2002), GAGgugaaaa (SEQ ID NO: 2003), GAGgugaaau (SEQ ID NO: 2004), GAGgugaaca (SEQ ID NO: 2005), GAGgugaagg (SEQ ID NO: 2006), GAGgugaaua (SEQ ID NO: 2007), GAGgugaauu (SEQ ID NO: 2008), GAGgugacau (SEQ ID NO: 2009), GAGgugacca (SEQ ID NO: 2010), GAGgugaccu (SEQ ID NO: 2011), GAGgugacua (SEQ ID NO: 2012), GAGgugacuu (SEQ ID NO: 2013), GAGgugagaa (SEQ ID NO: 2014), GAGgugagac (SEQ ID NO: 2015), GAGgugagag (SEQ ID NO: 2016), GAGgugagau (SEQ ID NO: 2017), GAGgugagca (SEQ ID NO: 2018), GAGgugagcc (SEQ ID NO: 2019), GAGgugagcg (SEQ ID NO: 2020), GAGgugagcu (SEQ ID NO: 2021), GAGgugagga (SEQ ID NO: 2022), GAGgugaggc (SEQ ID NO: 2023), GAGgugaggg (SEQ ID NO: 2024), GAGgugagua (SEQ ID NO: 2025), GAGgugagug (SEQ ID NO: 2026), GAGgugaguu (SEQ ID NO: 2027), GAGgugauau (SEQ ID NO: 2028), GAGgugaucc (SEQ ID NO: 2029), GAGgugaucu (SEQ ID NO: 2030), GAGgugauga (SEQ ID NO: 2031), GAGgugaugg (SEQ ID NO: 2032), GAGgugaugu (SEQ ID NO: 2033), GAGgugauuc (SEQ ID NO: 2034), GAGgugcaca (SEQ ID NO: 2035), GAGgugcaga (SEQ ID NO: 2036), GAGgugcagc (SEQ ID NO: 2037), GAGgugcagg (SEQ ID NO: 2038), GAGgugccag (SEQ ID NO: 2039), GAGgugccca (SEQ ID NO: 2040), GAGgugccuu (SEQ ID NO: 2041), GAGgugcggg (SEQ ID NO: 2042), GAGgugcgug (SEQ ID NO: 2043), GAGgugcucc (SEQ ID NO: 2044), GAGgugcugg (SEQ ID NO: 2045), GAGgugcuua (SEQ ID NO: 2046), GAGgugcuug (SEQ ID NO: 2047), GAGguggaaa (SEQ ID NO: 2048), GAGguggaau (SEQ ID NO: 2049), GAGguggacc (SEQ ID NO: 2050), GAGguggacg (SEQ ID NO: 2051), GAGguggagg (SEQ ID NO: 2052), GAGguggcug (SEQ ID NO: 2053), GAGgugggaa (SEQ ID NO: 2054), GAGgugggag (SEQ ID NO: 2055), GAGgugggau (SEQ ID NO: 2056), GAGgugggca (SEQ ID NO: 2057), GAGgugggcg (SEQ ID NO: 2058), GAGgugggcu (SEQ ID NO: 2059), GAGgugggga (SEQ ID NO: 2060), GAGguggggc (SEQ ID NO: 2061), GAGguggggg (SEQ ID NO: 2062), GAGgugggua (SEQ ID NO: 2063), GAGguggguc (SEQ ID NO: 2064), GAGgugggug (SEQ ID NO: 2065), GAGguggguu (SEQ ID NO: 2066), GAGgugguau (SEQ ID NO: 2067), GAGgugguuc (SEQ ID NO: 2068), GAGgugucau (SEQ ID NO: 2069), GAGgugugag (SEQ ID NO: 2070), GAGgugugau (SEQ ID NO: 2071), GAGgugugca (SEQ ID NO: 2072), GAGgugugcu (SEQ ID NO: 2073), GAGgugugga (SEQ ID NO: 2074), GAGguguggg (SEQ ID NO: 2075), GAGguguggu (SEQ ID NO: 2076), GAGgugugua (SEQ ID NO: 2077), GAGgugugug (SEQ ID NO: 2078), GAGguuaaau (SEQ ID NO: 2079), GAGguuaaga (SEQ ID NO: 2080), GAGguuaaua (SEQ ID NO: 2081), GAGguuaccg (SEQ ID NO: 2082), GAGguuagaa (SEQ ID NO: 2083), GAGguuagac (SEQ ID NO: 2084), GAGguuagag (SEQ ID NO: 2085), GAGguuaggu (SEQ ID NO: 2086), GAGguuagua (SEQ ID NO: 2087), GAGguuaguc (SEQ ID NO: 2088), GAGguuagug (SEQ ID NO: 2089), GAGguuaguu (SEQ ID NO: 2090), GAGguuaugu (SEQ ID NO: 2091), GAGguuauuc (SEQ ID NO: 2092), GAGguucaaa (SEQ ID NO: 2093), GAGguucaua (SEQ ID NO: 2094), GAGguucuga (SEQ ID NO: 2095), GAGguugaag (SEQ ID NO: 2096), GAGguugcag (SEQ ID NO: 2097), GAGguugcug (SEQ ID NO: 2098), GAGguuggaa (SEQ ID NO: 2099), GAGguuggag (SEQ ID NO: 2100), GAGguuggau (SEQ ID NO: 2101), GAGguuggua (SEQ ID NO: 2102), GAGguugguc (SEQ ID NO: 2103), GAGguugguu (SEQ ID NO: 2104), GAGguuguag (SEQ ID NO: 2105), GAGguuucug (SEQ ID NO: 2106), GAGguuugag (SEQ ID NO: 2107), GAGguuugga (SEQ ID NO: 2108), GAGguuuggg (SEQ ID NO: 2109), GAGguuugua (SEQ ID NO: 2110), GAGguuuguu (SEQ ID NO: 2111), GAGguuuuca (SEQ ID NO: 2112), GAGguuuuga (SEQ ID NO: 2113), GAGguuuugg (SEQ ID NO: 2114), GAGguuuuua (SEQ ID NO: 2115), GAGguuuuuc (SEQ ID NO: 2116), GAUguaaaau (SEQ ID NO: 2117), GAUguaagca (SEQ ID NO: 2118), GAUguaagcc (SEQ ID NO: 2119), GAUguaaggu (SEQ ID NO: 2120), GAUguaagua (SEQ ID NO: 2121), GAUguaagug (SEQ ID NO: 2122), GAUguaaguu (SEQ ID NO: 2123), GAUguacauc (SEQ ID NO: 2124), GAUguaggua (SEQ ID NO: 2125), GAUguauggc (SEQ ID NO: 2126), GAUguaugua (SEQ ID NO: 2127), GAUguauguu (SEQ ID NO: 2128), GAUgucagug (SEQ ID NO: 2129), GAUgugagag (SEQ ID NO: 2130), GAUgugagcc (SEQ ID NO: 2131), GAUgugagcu (SEQ ID NO: 2132), GAUgugagga (SEQ ID NO: 2133), GAUgugaguc (SEQ ID NO: 2134), GAUgugagug (SEQ ID NO: 2135), GAUgugaguu (SEQ ID NO: 2136), GAUgugggua (SEQ ID NO: 2137), GAUgugggug (SEQ ID NO: 2138), GAUguguguu (SEQ ID NO: 2139), GAUguuagcu (SEQ ID NO: 2140), GAUguucagu (SEQ ID NO: 2141), GAUguucgug (SEQ ID NO: 2142), GAUguuuguu (SEQ ID NO: 2143), GCAguaaagg (SEQ ID NO: 2144), GCAguaagaa (SEQ ID NO: 2145), GCAguaagga (SEQ ID NO: 2146), GCAguaagua (SEQ ID NO: 2147), GCAguaaguc (SEQ ID NO: 2148), GCAguaaguu (SEQ ID NO: 2149), GCAguagaug (SEQ ID NO: 2150), GCAguaggua (SEQ ID NO: 2151), GCAguaugug (SEQ ID NO: 2152), GCAguauguu (SEQ ID NO: 2153), GCAgucagua (SEQ ID NO: 2154), GCAgucagug (SEQ ID NO: 2155), GCAguccggu (SEQ ID NO: 2156), GCAgugacuu (SEQ ID NO: 2157), GCAgugagcc (SEQ ID NO: 2158), GCAgugagcg (SEQ ID NO: 2159), GCAgugagcu (SEQ ID NO: 2160), GCAgugagua (SEQ ID NO: 2161), GCAgugagug (SEQ ID NO: 2162), GCAgugaguu (SEQ ID NO: 2163), GCAgugggua (SEQ ID NO: 2164), GCAguuaagu (SEQ ID NO: 2165), GCAguugagu (SEQ ID NO: 2166), GCCguaaguc (SEQ ID NO: 2167), GCCgugagua (SEQ ID NO: 2168), GCGguaaagc (SEQ ID NO: 2169), GCGguaaaua (SEQ ID NO: 2170), GCGguaagcu (SEQ ID NO: 2171), GCGguaaggg (SEQ ID NO: 2172), GCGguaagug (SEQ ID NO: 2173), GCGguaauca (SEQ ID NO: 2174), GCGguacgua (SEQ ID NO: 2175), GCGguacuug (SEQ ID NO: 2176), GCGguagggu (SEQ ID NO: 2177), GCGguagugu (SEQ ID NO: 2178), GCGgugagca (SEQ ID NO: 2179), GCGgugagcu (SEQ ID NO: 2180), GCGgugaguu (SEQ ID NO: 2181), GCGguggcuc (SEQ ID NO: 2182), GCGgugugca (SEQ ID NO: 2183), GCGguguguu (SEQ ID NO: 2184), GCGguuaagu (SEQ ID NO: 2185), GCGguuugca (SEQ ID NO: 2186), GCUgcuguaa (SEQ ID NO: 2187), GCUguaaaua (SEQ ID NO: 2188), GCUguaagac (SEQ ID NO: 2189), GCUguaagag (SEQ ID NO: 2190), GCUguaagca (SEQ ID NO: 2191), GCUguaagga (SEQ ID NO: 2192), GCUguaagua (SEQ ID NO: 2193), GCUguaaguc (SEQ ID NO: 2194), GCUguaagug (SEQ ID NO: 2195), GCUguaaguu (SEQ ID NO: 2196), GCUguaggug (SEQ ID NO: 2197), GCUguauggu (SEQ ID NO: 2198), GCUgucagug (SEQ ID NO: 2199), GCUguccuug (SEQ ID NO: 2200), GCUgugagaa (SEQ ID NO: 2201), GCUgugagcc (SEQ ID NO: 2202), GCUgugagga (SEQ ID NO: 2203), GCUgugagua (SEQ ID NO: 2204), GCUgugaguc (SEQ ID NO: 2205), GCUgugagug (SEQ ID NO: 2206), GCUgugaguu (SEQ ID NO: 2207), GCUguggguu (SEQ ID NO: 2208), GGAguaagag (SEQ ID NO: 2209), GGAguaagca (SEQ ID NO: 2210), GGAguaagcc (SEQ ID NO: 2211), GGAguaagcu (SEQ ID NO: 2212), GGAguaagga (SEQ ID NO: 2213), GGAguaagug (SEQ ID NO: 2214), GGAguaaguu (SEQ ID NO: 2215), GGAguaauuu (SEQ ID NO: 2216), GGAguacugu (SEQ ID NO: 2217), GGAguaggaa (SEQ ID NO: 2218), GGAguaggua (SEQ ID NO: 2219), GGAguagguu (SEQ ID NO: 2220), GGAguaguau (SEQ ID NO: 2221), GGAguaugac (SEQ ID NO: 2222), GGAguauggu (SEQ ID NO: 2223), GGAgucaagu (SEQ ID NO: 2224), GGAgugaggg (SEQ ID NO: 2225), GGAgugagua (SEQ ID NO: 2226), GGAgugaguc (SEQ ID NO: 2227), GGAgugagug (SEQ ID NO: 2228), GGAgugaguu (SEQ ID NO: 2229), GGAgugcuuu (SEQ ID NO: 2230), GGAgugggca (SEQ ID NO: 2231), GGAgugggug (SEQ ID NO: 2232), GGAguuaagg (SEQ ID NO: 2233), GGAguugaga (SEQ ID NO: 2234), GGCguaagcc (SEQ ID NO: 2235), GGCguaggua (SEQ ID NO: 2236), GGCguaggug (SEQ ID NO: 2237), GGCgugagcc (SEQ ID NO: 2238), GGCgugaguc (SEQ ID NO: 2239), GGGguaaaca (SEQ ID NO: 2240), GGGguaaacc (SEQ ID NO: 2241), GGGguaaacu (SEQ ID NO: 2242), GGGguaagaa (SEQ ID NO: 2243), GGGguaagag (SEQ ID NO: 2244), GGGguaagau (SEQ ID NO: 2245), GGGguaagca (SEQ ID NO: 2246), GGGguaagcc (SEQ ID NO: 2247), GGGguaagcu (SEQ ID NO: 2248), GGGguaagga (SEQ ID NO: 2249), GGGguaaggg (SEQ ID NO: 2250), GGGguaagua (SEQ ID NO: 2251), GGGguaagug (SEQ ID NO: 2252), GGGguaaguu (SEQ ID NO: 2253), GGGguagaca (SEQ ID NO: 2254), GGGguaggag (SEQ ID NO: 2255), GGGguaggcc (SEQ ID NO: 2256), GGGguaggga (SEQ ID NO: 2257), GGGguaggua (SEQ ID NO: 2258), GGGguaggug (SEQ ID NO: 2259), GGGguagguu (SEQ ID NO: 2260), GGGguagugc (SEQ ID NO: 2261), GGGguaucug (SEQ ID NO: 2262), GGGguaugac (SEQ ID NO: 2263), GGGguaugga (SEQ ID NO: 2264), GGGguaugua (SEQ ID NO: 2265), GGGguauguc (SEQ ID NO: 2266), GGGguaugug (SEQ ID NO: 2267), GGGguauguu (SEQ ID NO: 2268), GGGgucagua (SEQ ID NO: 2269), GGGguccgug (SEQ ID NO: 2270), GGGgucggag (SEQ ID NO: 2271), GGGgucugug (SEQ ID NO: 2272), GGGgugaaca (SEQ ID NO: 2273), GGGgugaaga (SEQ ID NO: 2274), GGGgugagaa (SEQ ID NO: 2275), GGGgugagau (SEQ ID NO: 2276), GGGgugagcc (SEQ ID NO: 2277), GGGgugagcg (SEQ ID NO: 2278), GGGgugagcu (SEQ ID NO: 2279), GGGgugagga (SEQ ID NO: 2280), GGGgugaggc (SEQ ID NO: 2281), GGGgugaggg (SEQ ID NO: 2282), GGGgugaguc (SEQ ID NO: 2283), GGGgugagug (SEQ ID NO: 2284), GGGgugaguu (SEQ ID NO: 2285), GGGgugcgua (SEQ ID NO: 2286), GGGguggggu (SEQ ID NO: 2287), GGGgugggua (SEQ ID NO: 2288), GGGgugggug (SEQ ID NO: 2289), GGGguggguu (SEQ ID NO: 2290), GGGgugugcg (SEQ ID NO: 2291), GGGgugugua (SEQ ID NO: 2292), GGGguguguc (SEQ ID NO: 2293), GGGgugugug (SEQ ID NO: 2294), GGGguuacag (SEQ ID NO: 2295), GGGguuggac (SEQ ID NO: 2296), GGGguuggga (SEQ ID NO: 2297), GGGguuugcc (SEQ ID NO: 2298), GGGguuugua (SEQ ID NO: 2299), GGUguaagaa (SEQ ID NO: 2300), GGUguaagau (SEQ ID NO: 2301), GGUguaagca (SEQ ID NO: 2302), GGUguaagcc (SEQ ID NO: 2303), GGUguaagcg (SEQ ID NO: 2304), GGUguaaguc (SEQ ID NO: 2305), GGUguaagug (SEQ ID NO: 2306), GGUguagguc (SEQ ID NO: 2307), GGUguaggug (SEQ ID NO: 2308), GGUguagguu (SEQ ID NO: 2309), GGUguccgua (SEQ ID NO: 2310), GGUgugagag (SEQ ID NO: 2311), GGUgugagcc (SEQ ID NO: 2312), GGUgugagcu (SEQ ID NO: 2313), GGUgugagua (SEQ ID NO: 2314), GGUgugaguc (SEQ ID NO: 2315), GGUgugcuuc (SEQ ID NO: 2316), GGUguggcug (SEQ ID NO: 2317), GGUgugguga (SEQ ID NO: 2318), GGUgugucug (SEQ ID NO: 2319), GGUguugaaa (SEQ ID NO: 2320), GGUguugcug (SEQ ID NO: 2321), GUAguaagau (SEQ ID NO: 2322), GUAguaagua (SEQ ID NO: 2323), GUAguaagug (SEQ ID NO: 2324), GUAguagcuu (SEQ ID NO: 2325), GUAguaggua (SEQ ID NO: 2326), GUAgucagua (SEQ ID NO: 2327), GUAgugagua (SEQ ID NO: 2328), GUAguggugg (SEQ ID NO: 2329), GUAguuaagu (SEQ ID NO: 2330), GUAguuucug (SEQ ID NO: 2331), GUCguaagug (SEQ ID NO: 2332), GUCgugagug (SEQ ID NO: 2333), GUCgugaguu (SEQ ID NO: 2334), GUGgcaagua (SEQ ID NO: 2335), GUGgcuugua (SEQ ID NO: 2336), GUGguaaaau (SEQ ID NO: 2337), GUGguaaaga (SEQ ID NO: 2338), GUGguaaauu (SEQ ID NO: 2339), GUGguaacau (SEQ ID NO: 2340), GUGguaacua (SEQ ID NO: 2341), GUGguaagaa (SEQ ID NO: 2342), GUGguaagac (SEQ ID NO: 2343), GUGguaagag (SEQ ID NO: 2344), GUGguaagau (SEQ ID NO: 2345), GUGguaagca (SEQ ID NO: 2346), GUGguaagcg (SEQ ID NO: 2347), GUGguaagcu (SEQ ID NO: 2348), GUGguaagga (SEQ ID NO: 2349), GUGguaaggc (SEQ ID NO: 2350), GUGguaagua (SEQ ID NO: 2351), GUGguaaguc (SEQ ID NO: 2352), GUGguaagug (SEQ ID NO: 2353), GUGguaaguu (SEQ ID NO: 2354), GUGguaauga (SEQ ID NO: 2355), GUGguaauuc (SEQ ID NO: 2356), GUGguaauuu (SEQ ID NO: 2357), GUGguacaug (SEQ ID NO: 2358), GUGguacgau (SEQ ID NO: 2359), GUGguacuau (SEQ ID NO: 2360), GUGguacuug (SEQ ID NO: 2361), GUGguagaua (SEQ ID NO: 2362), GUGguagege (SEQ ID NO: 2363), GUGguaggga (SEQ ID NO: 2364), GUGguagguc (SEQ ID NO: 2365), GUGguaggug (SEQ ID NO: 2366), GUGguagguu (SEQ ID NO: 2367), GUGguauaaa (SEQ ID NO: 2368), GUGguaucuc (SEQ ID NO: 2369), GUGguaugaa (SEQ ID NO: 2370), GUGguaugau (SEQ ID NO: 2371), GUGguaugca (SEQ ID NO: 2372), GUGguaugua (SEQ ID NO: 2373), GUGguauguu (SEQ ID NO: 2374), GUGguccgug (SEQ ID NO: 2375), GUGgucuggc (SEQ ID NO: 2376), GUGgugaaac (SEQ ID NO: 2377), GUGgugagaa (SEQ ID NO: 2378), GUGgugagau (SEQ ID NO: 2379), GUGgugagca (SEQ ID NO: 2380), GUGgugagcu (SEQ ID NO: 2381), GUGgugagga (SEQ ID NO: 2382), GUGgugaggc (SEQ ID NO: 2383), GUGgugagug (SEQ ID NO: 2384), GUGgugaguu (SEQ ID NO: 2385), GUGgugauua (SEQ ID NO: 2386), GUGgugauuc (SEQ ID NO: 2387), GUGgugcgau (SEQ ID NO: 2388), GUGgugcuua (SEQ ID NO: 2389), GUGgugggaa (SEQ ID NO: 2390), GUGgugggua (SEQ ID NO: 2391), GUGguggguc (SEQ ID NO: 2392), GUGguguccg (SEQ ID NO: 2393), GUGguuagca (SEQ ID NO: 2394), GUGguuaggu (SEQ ID NO: 2395), GUGguuagug (SEQ ID NO: 2396), GUGguuugca (SEQ ID NO: 2397), GUGguuugua (SEQ ID NO: 2398), GUUguaaggu (SEQ ID NO: 2399), GUUguaagua (SEQ ID NO: 2400), GUUguaaguc (SEQ ID NO: 2401), GUUguaaguu (SEQ ID NO: 2402), GUUguaccac (SEQ ID NO: 2403), GUUguagcgu (SEQ ID NO: 2404), GUUguaugug (SEQ ID NO: 2405), GUUguauguu (SEQ ID NO: 2406), GUUgucugug (SEQ ID NO: 2407), GUUgugagcu (SEQ ID NO: 2408), GUUgugagug (SEQ ID NO: 2409), GUUgugaguu (SEQ ID NO: 2410), GUUgugggua (SEQ ID NO: 2411), GUUguggguu (SEQ ID NO: 2412), UAAguaaaug (SEQ ID NO: 2413), UAAguaacua (SEQ ID NO: 2414), UAAguaagaa (SEQ ID NO: 2415), UAAguaagag (SEQ ID NO: 2416), UAAguaagau (SEQ ID NO: 2417), UAAguaagca (SEQ ID NO: 2418), UAAguaagcu (SEQ ID NO: 2419), UAAguaagga (SEQ ID NO: 2420), UAAguaaggu (SEQ ID NO: 2421), UAAguaagua (SEQ ID NO: 2422), UAAguaaguc (SEQ ID NO: 2423), UAAguaagug (SEQ ID NO: 2424), UAAguaaguu (SEQ ID NO: 2425), UAAguaauaa (SEQ ID NO: 2426), UAAguacuag (SEQ ID NO: 2427), UAAguaguuu (SEQ ID NO: 2428), UAAguauaaa (SEQ ID NO: 2429), UAAguauaca (SEQ ID NO: 2430), UAAguaugua (SEQ ID NO: 2431), UAAguauuau (SEQ ID NO: 2432), UAAguauuuu (SEQ ID NO: 2433), UAAgucuuuu (SEQ ID NO: 2434), UAAgugagac (SEQ ID NO: 2435), UAAgugagga (SEQ ID NO: 2436), UAAgugaggg (SEQ ID NO: 2437), UAAgugagua (SEQ ID NO: 2438), UAAgugaguc (SEQ ID NO: 2439), UAAgugagug (SEQ ID NO: 2440), UAAgugaguu (SEQ ID NO: 2441), UAAgugaucc (SEQ ID NO: 2442), UAAgugauuc (SEQ ID NO: 2443), UAAgugcgug (SEQ ID NO: 2444), UAAguuaagu (SEQ ID NO: 2445), UAAguuccag (SEQ ID NO: 2446), UAAguucuuu (SEQ ID NO: 2447), UAAguuguaa (SEQ ID NO: 2448), UAAguuguau (SEQ ID NO: 2449), UAAguuuguu (SEQ ID NO: 2450), UACguaacug (SEQ ID NO: 2451), UACguaagaa (SEQ ID NO: 2452), UACguaagau (SEQ ID NO: 2453), UACguaagua (SEQ ID NO: 2454), UACguaagug (SEQ ID NO: 2455), UACguauccu (SEQ ID NO: 2456), UACgucuggc (SEQ ID NO: 2457), UACgugacca (SEQ ID NO: 2458), UAGgcaagac (SEQ ID NO: 2459), UAGgcaaguc (SEQ ID NO: 2460), UAGgcagguc (SEQ ID NO: 2461), UAGgcgugug (SEQ ID NO: 2462), UAGguaaaaa (SEQ ID NO: 2463), UAGguaaaac (SEQ ID NO: 2464), UAGguaaaag (SEQ ID NO: 2465), UAGguaaaau (SEQ ID NO: 2466), UAGguaaaca (SEQ ID NO: 2467), UAGguaaaga (SEQ ID NO: 2468), UAGguaaaua (SEQ ID NO: 2469), UAGguaaauc (SEQ ID NO: 2470), UAGguaaaug (SEQ ID NO: 2471), UAGguaaauu (SEQ ID NO: 2472), UAGguaacac (SEQ ID NO: 2473), UAGguaacag (SEQ ID NO: 2474), UAGguaacau (SEQ ID NO: 2475), UAGguaacca (SEQ ID NO: 2476), UAGguaacgg (SEQ ID NO: 2477), UAGguaacua (SEQ ID NO: 2478), UAGguaacuc (SEQ ID NO: 2479), UAGguaacug (SEQ ID NO: 2480), UAGguaacuu (SEQ ID NO: 2481), UAGguaagac (SEQ ID NO: 2482), UAGguaagag (SEQ ID NO: 2483), UAGguaagau (SEQ ID NO: 2484), UAGguaagca (SEQ ID NO: 2485), UAGguaagcc (SEQ ID NO: 2486), UAGguaagcu (SEQ ID NO: 2487), UAGguaagga (SEQ ID NO: 2488), UAGguaaggc (SEQ ID NO: 2489), UAGguaaggg (SEQ ID NO: 2490), UAGguaagua (SEQ ID NO: 2491), UAGguaaguc (SEQ ID NO: 2492), UAGguaagug (SEQ ID NO: 2493), UAGguaaguu (SEQ ID NO: 2494), UAGguaauag (SEQ ID NO: 2495), UAGguaauau (SEQ ID NO: 2496), UAGguaaucu (SEQ ID NO: 2497), UAGguaauga (SEQ ID NO: 2498), UAGguaaugg (SEQ ID NO: 2499), UAGguaaugu (SEQ ID NO: 2500), UAGguaauua (SEQ ID NO: 2501), UAGguaauuc (SEQ ID NO: 2502), UAGguaauuu (SEQ ID NO: 2503), UAGguacagc (SEQ ID NO: 2504), UAGguacagu (SEQ ID NO: 2505), UAGguacauu (SEQ ID NO: 2506), UAGguaccag (SEQ ID NO: 2507), UAGguaccua (SEQ ID NO: 2508), UAGguaccuu (SEQ ID NO: 2509), UAGguacgag (SEQ ID NO: 2510), UAGguacgua (SEQ ID NO: 2511), UAGguacguu (SEQ ID NO: 2512), UAGguacuau (SEQ ID NO: 2513), UAGguacuga (SEQ ID NO: 2514), UAGguacugg (SEQ ID NO: 2515), UAGguacuuc (SEQ ID NO: 2516), UAGguacuuu (SEQ ID NO: 2517), UAGguagcgg (SEQ ID NO: 2518), UAGguaggaa (SEQ ID NO: 2519), UAGguaggac (SEQ ID NO: 2520), UAGguaggau (SEQ ID NO: 2521), UAGguaggga (SEQ ID NO: 2522), UAGguagggg (SEQ ID NO: 2523), UAGguaggua (SEQ ID NO: 2524), UAGguagguc (SEQ ID NO: 2525), UAGguaggug (SEQ ID NO: 2526), UAGguagguu (SEQ ID NO: 2527), UAGguaguaa (SEQ ID NO: 2528), UAGguagucu (SEQ ID NO: 2529), UAGguagugg (SEQ ID NO: 2530), UAGguagugu (SEQ ID NO: 2531), UAGguaguuu (SEQ ID NO: 2532), UAGguauaaa (SEQ ID NO: 2533), UAGguauaac (SEQ ID NO: 2534), UAGguauaag (SEQ ID NO: 2535), UAGguauaau (SEQ ID NO: 2536), UAGguauaca (SEQ ID NO: 2537), UAGguauacu (SEQ ID NO: 2538), UAGguauaua (SEQ ID NO: 2539), UAGguauauc (SEQ ID NO: 2540), UAGguauauu (SEQ ID NO: 2541), UAGguaucag (SEQ ID NO: 2542), UAGguaucua (SEQ ID NO: 2543), UAGguaucuc (SEQ ID NO: 2544), UAGguaugaa (SEQ ID NO: 2545), UAGguaugag (SEQ ID NO: 2546), UAGguaugca (SEQ ID NO: 2547), UAGguaugga (SEQ ID NO: 2548), UAGguauggc (SEQ ID NO: 2549), UAGguauggu (SEQ ID NO: 2550), UAGguaugua (SEQ ID NO: 2551), UAGguauguc (SEQ ID NO: 2552), UAGguaugug (SEQ ID NO: 2553), UAGguauguu (SEQ ID NO: 2554), UAGguauuaa (SEQ ID NO: 2555), UAGguauuac (SEQ ID NO: 2556), UAGguauuau (SEQ ID NO: 2557), UAGguauuca (SEQ ID NO: 2558), UAGguauucc (SEQ ID NO: 2559), UAGguauucu (SEQ ID NO: 2560), UAGguauuga (SEQ ID NO: 2561), UAGguauuua (SEQ ID NO: 2562), UAGguauuuc (SEQ ID NO: 2563), UAGguauuuu (SEQ ID NO: 2564), UAGgucacuc (SEQ ID NO: 2565), UAGgucagcu (SEQ ID NO: 2566), UAGgucaggu (SEQ ID NO: 2567), UAGgucagua (SEQ ID NO: 2568), UAGgucagug (SEQ ID NO: 2569), UAGgucaguu (SEQ ID NO: 2570), UAGgucaucu (SEQ ID NO: 2571), UAGgucauug (SEQ ID NO: 2572), UAGguccaau (SEQ ID NO: 2573), UAGguccugu (SEQ ID NO: 2574), UAGgucucaa (SEQ ID NO: 2575), UAGgucucgc (SEQ ID NO: 2576), UAGgucuggc (SEQ ID NO: 2577), UAGgucuguc (SEQ ID NO: 2578), UAGgucugug (SEQ ID NO: 2579), UAGgugaagu (SEQ ID NO: 2580), UAGgugaaua (SEQ ID NO: 2581), UAGgugaaug (SEQ ID NO: 2582), UAGgugaauu (SEQ ID NO: 2583), UAGgugacau (SEQ ID NO: 2584), UAGgugacca (SEQ ID NO: 2585), UAGgugacua (SEQ ID NO: 2586), UAGgugagaa (SEQ ID NO: 2587), UAGgugagac (SEQ ID NO: 2588), UAGgugagag (SEQ ID NO: 2589), UAGgugagau (SEQ ID NO: 2590), UAGgugagcc (SEQ ID NO: 2591), UAGgugagcu (SEQ ID NO: 2592), UAGgugagga (SEQ ID NO: 2593), UAGgugaggc (SEQ ID NO: 2594), UAGgugaggu (SEQ ID NO: 2595), UAGgugagua (SEQ ID NO: 2596), UAGgugaguc (SEQ ID NO: 2597), UAGgugagug (SEQ ID NO: 2598), UAGgugauca (SEQ ID NO: 2599), UAGgugauuc (SEQ ID NO: 2600), UAGgugauuu (SEQ ID NO: 2601), UAGgugcaua (SEQ ID NO: 2602), UAGgugcauc (SEQ ID NO: 2603), UAGgugccgu (SEQ ID NO: 2604), UAGgugccug (SEQ ID NO: 2605), UAGgugcgca (SEQ ID NO: 2606), UAGgugcgua (SEQ ID NO: 2607), UAGgugcgug (SEQ ID NO: 2608), UAGgugcuga (SEQ ID NO: 2609), UAGguggaua (SEQ ID NO: 2610), UAGgugggaa (SEQ ID NO: 2611), UAGgugggac (SEQ ID NO: 2612), UAGgugggag (SEQ ID NO: 2613), UAGgugggau (SEQ ID NO: 2614), UAGgugggcc (SEQ ID NO: 2615), UAGgugggcu (SEQ ID NO: 2616), UAGguggguu (SEQ ID NO: 2617), UAGguggugu (SEQ ID NO: 2618), UAGguguaaa (SEQ ID NO: 2619), UAGgugugaa (SEQ ID NO: 2620), UAGgugugag (SEQ ID NO: 2621), UAGgugugca (SEQ ID NO: 2622), UAGgugugcc (SEQ ID NO: 2623), UAGgugugcg (SEQ ID NO: 2624), UAGguguggu (SEQ ID NO: 2625), UAGgugugua (SEQ ID NO: 2626), UAGgugugug (SEQ ID NO: 2627), UAGguguugg (SEQ ID NO: 2628), UAGguuaagc (SEQ ID NO: 2629), UAGguuagac (SEQ ID NO: 2630), UAGguuagcc (SEQ ID NO: 2631), UAGguuaggc (SEQ ID NO: 2632), UAGguuagua (SEQ ID NO: 2633), UAGguuaguc (SEQ ID NO: 2634), UAGguuagug (SEQ ID NO: 2635), UAGguucccc (SEQ ID NO: 2636), UAGguucuac (SEQ ID NO: 2637), UAGguuggua (SEQ ID NO: 2638), UAGguugguu (SEQ ID NO: 2639), UAGguugucc (SEQ ID NO: 2640), UAGguuuauu (SEQ ID NO: 2641), UAGguuugcc (SEQ ID NO: 2642), UAGguuugua (SEQ ID NO: 2643), UAGguuuguc (SEQ ID NO: 2644), UAGguuugug (SEQ ID NO: 2645), UAGguuuguu (SEQ ID NO: 2646), UAGguuuuuc (SEQ ID NO: 2647), UAGguuuuug (SEQ ID NO: 2648), UAUguaagaa (SEQ ID NO: 2649), UAUguaagau (SEQ ID NO: 2650), UAUguaagca (SEQ ID NO: 2651), UAUguaagcc (SEQ ID NO: 2652), UAUguaagua (SEQ ID NO: 2653), UAUguaaguc (SEQ ID NO: 2654), UAUguaagug (SEQ ID NO: 2655), UAUguaaguu (SEQ ID NO: 2656), UAUguacgug (SEQ ID NO: 2657), UAUguacguu (SEQ ID NO: 2658), UAUguagguc (SEQ ID NO: 2659), UAUguagguu (SEQ ID NO: 2660), UAUguauccu (SEQ ID NO: 2661), UAUguaucuc (SEQ ID NO: 2662), UAUguaugua (SEQ ID NO: 2663), UAUguauguc (SEQ ID NO: 2664), UAUguaugug (SEQ ID NO: 2665), UAUguauuau (SEQ ID NO: 2666), UAUgucagaa (SEQ ID NO: 2667), UAUgucugua (SEQ ID NO: 2668), UAUgugaaua (SEQ ID NO: 2669), UAUgugacag (SEQ ID NO: 2670), UAUgugagua (SEQ ID NO: 2671), UAUgugagug (SEQ ID NO: 2672), UAUgugaguu (SEQ ID NO: 2673), UAUgugggca (SEQ ID NO: 2674), UAUgugugua (SEQ ID NO: 2675), UAUguguuua (SEQ ID NO: 2676), UAUguuuugu (SEQ ID NO: 2677), UCAgcgacau (SEQ ID NO: 2678), UCAguaaaau (SEQ ID NO: 2679), UCAguaaaua (SEQ ID NO: 2680), UCAguaacug (SEQ ID NO: 2681), UCAguaagaa (SEQ ID NO: 2682), UCAguaagag (SEQ ID NO: 2683), UCAguaagau (SEQ ID NO: 2684), UCAguaagca (SEQ ID NO: 2685), UCAguaagcc (SEQ ID NO: 2686), UCAguaagcu (SEQ ID NO: 2687), UCAguaaggg (SEQ ID NO: 2688), UCAguaagua (SEQ ID NO: 2689), UCAguaaguc (SEQ ID NO: 2690), UCAguaagug (SEQ ID NO: 2691), UCAguaaguu (SEQ ID NO: 2692), UCAguaucuu (SEQ ID NO: 2693), UCAguaugga (SEQ ID NO: 2694), UCAguauggu (SEQ ID NO: 2695), UCAgucccca (SEQ ID NO: 2696), UCAgugagca (SEQ ID NO: 2697), UCAgugagcu (SEQ ID NO: 2698), UCAgugagua (SEQ ID NO: 2699), UCAgugagug (SEQ ID NO: 2700), UCAgugaguu (SEQ ID NO: 2701), UCAgugauug (SEQ ID NO: 2702), UCAgugggug (SEQ ID NO: 2703), UCAguugagc (SEQ ID NO: 2704), UCAguugauu (SEQ ID NO: 2705), UCAguuuagu (SEQ ID NO: 2706), UCCguaagca (SEQ ID NO: 2707), UCCguaagcu (SEQ ID NO: 2708), UCCguaaguc (SEQ ID NO: 2709), UCCguaagug (SEQ ID NO: 2710), UCCguaauag (SEQ ID NO: 2711), UCCguacuua (SEQ ID NO: 2712), UCCguaugua (SEQ ID NO: 2713), UCCguauguu (SEQ ID NO: 2714), UCCgugagau (SEQ ID NO: 2715), UCCgugaguc (SEQ ID NO: 2716), UCGguaaauu (SEQ ID NO: 2717), UCGguaagag (SEQ ID NO: 2718), UCGguaagcu (SEQ ID NO: 2719), UCGguacauc (SEQ ID NO: 2720), UCGguacucc (SEQ ID NO: 2721), UCGguagacc (SEQ ID NO: 2722), UCGguagguu (SEQ ID NO: 2723), UCGguaguaa (SEQ ID NO: 2724), UCGguaugug (SEQ ID NO: 2725), UCGguauguu (SEQ ID NO: 2726), UCGguauuga (SEQ ID NO: 2727), UCGgucagua (SEQ ID NO: 2728), UCGgucuuag (SEQ ID NO: 2729), UCGgugaagu (SEQ ID NO: 2730), UCGgugagaa (SEQ ID NO: 2731), UCGgugagca (SEQ ID NO: 2732), UCGgugaggc (SEQ ID NO: 2733), UCGgugagua (SEQ ID NO: 2734), UCGgugcgcu (SEQ ID NO: 2735), UCGgugcuuu (SEQ ID NO: 2736), UCGgugguuu (SEQ ID NO: 2737), UCGguuagcu (SEQ ID NO: 2738), UCUguaaaag (SEQ ID NO: 2739), UCUguaagaa (SEQ ID NO: 2740), UCUguaagau (SEQ ID NO: 2741), UCUguaagca (SEQ ID NO: 2742), UCUguaagcu (SEQ ID NO: 2743), UCUguaagua (SEQ ID NO: 2744), UCUguaaguc (SEQ ID NO: 2745), UCUguaagug (SEQ ID NO: 2746), UCUguaaguu (SEQ ID NO: 2747), UCUguaauaa (SEQ ID NO: 2748), UCUguaauga (SEQ ID NO: 2749), UCUguaaugu (SEQ ID NO: 2750), UCUguaggua (SEQ ID NO: 2751), UCUguagguu (SEQ ID NO: 2752), UCUguauaua (SEQ ID NO: 2753), UCUguaugac (SEQ ID NO: 2754), UCUguaugua (SEQ ID NO: 2755), UCUguccueg (SEQ ID NO: 2756), UCUgugagag (SEQ ID NO: 2757), UCUgugagcu (SEQ ID NO: 2758), UCUgugagga (SEQ ID NO: 2759), UCUgugagua (SEQ ID NO: 2760), UCUgugaguc (SEQ ID NO: 2761), UCUgugagug (SEQ ID NO: 2762), UCUgugaguu (SEQ ID NO: 2763), UCUgugcgua (SEQ ID NO: 2764), UCUgugugag (SEQ ID NO: 2765), UGAguaacuu (SEQ ID NO: 2766), UGAguaagau (SEQ ID NO: 2767), UGAguaagca (SEQ ID NO: 2768), UGAguaagcu (SEQ ID NO: 2769), UGAguaaggc (SEQ ID NO: 2770), UGAguaaggu (SEQ ID NO: 2771), UGAguaagua (SEQ ID NO: 2772), UGAguaaguc (SEQ ID NO: 2773), UGAguaagug (SEQ ID NO: 2774), UGAguaaguu (SEQ ID NO: 2775), UGAguaaucc (SEQ ID NO: 2776), UGAguaauua (SEQ ID NO: 2777), UGAguacagu (SEQ ID NO: 2778), UGAguacgua (SEQ ID NO: 2779), UGAguacguu (SEQ ID NO: 2780), UGAguacugu (SEQ ID NO: 2781), UGAguagcug (SEQ ID NO: 2782), UGAguaggua (SEQ ID NO: 2783), UGAguauaaa (SEQ ID NO: 2784), UGAguaugcu (SEQ ID NO: 2785), UGAguaugga (SEQ ID NO: 2786), UGAguaugua (SEQ ID NO: 2787), UGAguauguc (SEQ ID NO: 2788), UGAguauguu (SEQ ID NO: 2789), UGAgucagag (SEQ ID NO: 2790), UGAgucuacg (SEQ ID NO: 2791), UGAgugaaua (SEQ ID NO: 2792), UGAgugaauu (SEQ ID NO: 2793), UGAgugagaa (SEQ ID NO: 2794), UGAgugagau (SEQ ID NO: 2795), UGAgugagca (SEQ ID NO: 2796), UGAgugagcc (SEQ ID NO: 2797), UGAgugagga (SEQ ID NO: 2798), UGAgugagua (SEQ ID NO: 2799), UGAgugagug (SEQ ID NO: 2800), UGAgugaguu (SEQ ID NO: 2801), UGAgugggaa (SEQ ID NO: 2802), UGAguuaaga (SEQ ID NO: 2803), UGAguuaaug (SEQ ID NO: 2804), UGAguuacgg (SEQ ID NO: 2805), UGAguuaggu (SEQ ID NO: 2806), UGAguucuau (SEQ ID NO: 2807), UGAguugguu (SEQ ID NO: 2808), UGAguuguag (SEQ ID NO: 2809), UGAguuuauc (SEQ ID NO: 2810), UGCguaaguc (SEQ ID NO: 2811), UGCguaagug (SEQ ID NO: 2812), UGCguacggc (SEQ ID NO: 2813), UGCguacggg (SEQ ID NO: 2814), UGCguaugua (SEQ ID NO: 2815), UGGgcaaguc (SEQ ID NO: 2816), UGGgcaagug (SEQ ID NO: 2817), UGGgcacauc (SEQ ID NO: 2818), UGGgccacgu (SEQ ID NO: 2819), UGGgccccgg (SEQ ID NO: 2820), UGGguaaaau (SEQ ID NO: 2821), UGGguaaagc (SEQ ID NO: 2822), UGGguaaagg (SEQ ID NO: 2823), UGGguaaagu (SEQ ID NO: 2824), UGGguaaaua (SEQ ID NO: 2825), UGGguaaaug (SEQ ID NO: 2826), UGGguaaauu (SEQ ID NO: 2827), UGGguaacag (SEQ ID NO: 2828), UGGguaacau (SEQ ID NO: 2829), UGGguaacua (SEQ ID NO: 2830), UGGguaacuu (SEQ ID NO: 2831), UGGguaagaa (SEQ ID NO: 2832), UGGguaagac (SEQ ID NO: 2833), UGGguaagag (SEQ ID NO: 2834), UGGguaagau (SEQ ID NO: 2835), UGGguaagca (SEQ ID NO: 2836), UGGguaagcc (SEQ ID NO: 2837), UGGguaagcu (SEQ ID NO: 2838), UGGguaaggg (SEQ ID NO: 2839), UGGguaaggu (SEQ ID NO: 2840), UGGguaagua (SEQ ID NO: 2841), UGGguaaguc (SEQ ID NO: 2842), UGGguaagug (SEQ ID NO: 2843), UGGguaaguu (SEQ ID NO: 2844), UGGguaaugu (SEQ ID NO: 2845), UGGguaauua (SEQ ID NO: 2846), UGGguaauuu (SEQ ID NO: 2847), UGGguacaaa (SEQ ID NO: 2848), UGGguacagu (SEQ ID NO: 2849), UGGguacuac (SEQ ID NO: 2850), UGGguaggga (SEQ ID NO: 2851), UGGguagguc (SEQ ID NO: 2852), UGGguaggug (SEQ ID NO: 2853), UGGguagguu (SEQ ID NO: 2854), UGGguaguua (SEQ ID NO: 2855), UGGguauagu (SEQ ID NO: 2856), UGGguaugaa (SEQ ID NO: 2857), UGGguaugac (SEQ ID NO: 2858), UGGguaugag (SEQ ID NO: 2859), UGGguaugua (SEQ ID NO: 2860), UGGguauguc (SEQ ID NO: 2861), UGGguaugug (SEQ ID NO: 2862), UGGguauguu (SEQ ID NO: 2863), UGGguauuug (SEQ ID NO: 2864), UGGgucuuug (SEQ ID NO: 2865), UGGgugaccu (SEQ ID NO: 2866), UGGgugacua (SEQ ID NO: 2867), UGGgugagac (SEQ ID NO: 2868), UGGgugagag (SEQ ID NO: 2869), UGGgugagca (SEQ ID NO: 2870), UGGgugagcc (SEQ ID NO: 2871), UGGgugagga (SEQ ID NO: 2872), UGGgugaggc (SEQ ID NO: 2873), UGGgugaggg (SEQ ID NO: 2874), UGGgugagua (SEQ ID NO: 2875), UGGgugaguc (SEQ ID NO: 2876), UGGgugagug (SEQ ID NO: 2877), UGGgugaguu (SEQ ID NO: 2878), UGGgugcgug (SEQ ID NO: 2879), UGGguggagg (SEQ ID NO: 2880), UGGguggcuu (SEQ ID NO: 2881), UGGguggggg (SEQ ID NO: 2882), UGGgugggua (SEQ ID NO: 2883), UGGguggguc (SEQ ID NO: 2884), UGGgugggug (SEQ ID NO: 2885), UGGguggguu (SEQ ID NO: 2886), UGGgugugga (SEQ ID NO: 2887), UGGguguguc (SEQ ID NO: 2888), UGGgugugug (SEQ ID NO: 2889), UGGguguguu (SEQ ID NO: 2890), UGGguguuua (SEQ ID NO: 2891), UGGguuaaug (SEQ ID NO: 2892), UGGguuaguc (SEQ ID NO: 2893), UGGguuagug (SEQ ID NO: 2894), UGGguuaguu (SEQ ID NO: 2895), UGGguucaag (SEQ ID NO: 2896), UGGguucgua (SEQ ID NO: 2897), UGGguuggug (SEQ ID NO: 2898), UGGguuuaag (SEQ ID NO: 2899), UGGguuugua (SEQ ID NO: 2900), UGUgcaagua (SEQ ID NO: 2901), UGUguaaaua (SEQ ID NO: 2902), UGUguaagaa (SEQ ID NO: 2903), UGUguaagac (SEQ ID NO: 2904), UGUguaagag (SEQ ID NO: 2905), UGUguaaggu (SEQ ID NO: 2906), UGUguaagua (SEQ ID NO: 2907), UGUguaaguc (SEQ ID NO: 2908), UGUguaaguu (SEQ ID NO: 2909), UGUguacuuc (SEQ ID NO: 2910), UGUguaggeg (SEQ ID NO: 2911), UGUguaggua (SEQ ID NO: 2912), UGUguaguua (SEQ ID NO: 2913), UGUguaugug (SEQ ID NO: 2914), UGUgucagua (SEQ ID NO: 2915), UGUgucugua (SEQ ID NO: 2916), UGUgucuguc (SEQ ID NO: 2917), UGUgugaccc (SEQ ID NO: 2918), UGUgugagau (SEQ ID NO: 2919), UGUgugagca (SEQ ID NO: 2920), UGUgugagcc (SEQ ID NO: 2921), UGUgugagua (SEQ ID NO: 2922), UGUgugaguc (SEQ ID NO: 2923), UGUgugagug (SEQ ID NO: 2924), UGUgugcgug (SEQ ID NO: 2925), UGUgugggug (SEQ ID NO: 2926), UGUguggguu (SEQ ID NO: 2927), UGUgugugag (SEQ ID NO: 2928), UGUguguucu (SEQ ID NO: 2929), UGUguuuaga (SEQ ID NO: 2930), UUAguaaaua (SEQ ID NO: 2931), UUAguaagaa (SEQ ID NO: 2932), UUAguaagua (SEQ ID NO: 2933), UUAguaagug (SEQ ID NO: 2934), UUAguaaguu (SEQ ID NO: 2935), UUAguaggug (SEQ ID NO: 2936), UUAgugagca (SEQ ID NO: 2937), UUAgugaguu (SEQ ID NO: 2938), UUAguuaagu (SEQ ID NO: 2939), UUCguaaguc (SEQ ID NO: 2940), UUCguaaguu (SEQ ID NO: 2941), UUCguaauua (SEQ ID NO: 2942), UUCgugagua (SEQ ID NO: 2943), UUCgugaguu (SEQ ID NO: 2944), UUGgcaagug (SEQ ID NO: 2945), UUGgccgagu (SEQ ID NO: 2946), UUGguaaaaa (SEQ ID NO: 2947), UUGguaaaau (SEQ ID NO: 2948), UUGguaaaga (SEQ ID NO: 2949), UUGguaaagg (SEQ ID NO: 2950), UUGguaaagu (SEQ ID NO: 2951), UUGguaaauc (SEQ ID NO: 2952), UUGguaaaug (SEQ ID NO: 2953), UUGguaaauu (SEQ ID NO: 2954), UUGguaacug (SEQ ID NO: 2955), UUGguaacuu (SEQ ID NO: 2956), UUGguaagaa (SEQ ID NO: 2957), UUGguaagag (SEQ ID NO: 2958), UUGguaagcu (SEQ ID NO: 2959), UUGguaagga (SEQ ID NO: 2960), UUGguaaggg (SEQ ID NO: 2961), UUGguaagua (SEQ ID NO: 2962), UUGguaagug (SEQ ID NO: 2963), UUGguaaguu (SEQ ID NO: 2964), UUGguaauac (SEQ ID NO: 2965), UUGguaauca (SEQ ID NO: 2966), UUGguaaugc (SEQ ID NO: 2967), UUGguaaugu (SEQ ID NO: 2968), UUGguaauug (SEQ ID NO: 2969), UUGguaauuu (SEQ ID NO: 2970), UUGguacaua (SEQ ID NO: 2971), UUGguacgug (SEQ ID NO: 2972), UUGguagagg (SEQ ID NO: 2973), UUGguaggac (SEQ ID NO: 2974), UUGguaggcg (SEQ ID NO: 2975), UUGguaggcu (SEQ ID NO: 2976), UUGguaggga (SEQ ID NO: 2977), UUGguaggua (SEQ ID NO: 2978), UUGguagguc (SEQ ID NO: 2979), UUGguaggug (SEQ ID NO: 2980), UUGguauaaa (SEQ ID NO: 2981), UUGguauaca (SEQ ID NO: 2982), UUGguauauu (SEQ ID NO: 2983), UUGguaucua (SEQ ID NO: 2984), UUGguaucuc (SEQ ID NO: 2985), UUGguaugca (SEQ ID NO: 2986), UUGguaugua (SEQ ID NO: 2987), UUGguaugug (SEQ ID NO: 2988), UUGguauguu (SEQ ID NO: 2989), UUGguauugu (SEQ ID NO: 2990), UUGguauuua (SEQ ID NO: 2991), UUGguauuuu (SEQ ID NO: 2992), UUGgucagaa (SEQ ID NO: 2993), UUGgucagua (SEQ ID NO: 2994), UUGgucucug (SEQ ID NO: 2995), UUGgucugca (SEQ ID NO: 2996), UUGgugaaaa (SEQ ID NO: 2997), UUGgugacug (SEQ ID NO: 2998), UUGgugagac (SEQ ID NO: 2999), UUGgugagau (SEQ ID NO: 3000), UUGgugagca (SEQ ID NO: 3001), UUGgugagga (SEQ ID NO: 3002), UUGgugaggg (SEQ ID NO: 3003), UUGgugagua (SEQ ID NO: 3004), UUGgugaguc (SEQ ID NO: 3005), UUGgugagug (SEQ ID NO: 3006), UUGgugaguu (SEQ ID NO: 3007), UUGgugaugg (SEQ ID NO: 3008), UUGgugauua (SEQ ID NO: 3009), UUGgugauug (SEQ ID NO: 3010), UUGgugcaca (SEQ ID NO: 3011), UUGgugggaa (SEQ ID NO: 3012), UUGguggggc (SEQ ID NO: 3013), UUGgugggua (SEQ ID NO: 3014), UUGguggguc (SEQ ID NO: 3015), UUGgugggug (SEQ ID NO: 3016), UUGguggguu (SEQ ID NO: 3017), UUGguguggu (SEQ ID NO: 3018), UUGguguguc (SEQ ID NO: 3019), UUGgugugug (SEQ ID NO: 3020), UUGguguguu (SEQ ID NO: 3021), UUGguuaagu (SEQ ID NO: 3022), UUGguuagca (SEQ ID NO: 3023), UUGguuagug (SEQ ID NO: 3024), UUGguuaguu (SEQ ID NO: 3025), UUGguuggga (SEQ ID NO: 3026), UUGguugguu (SEQ ID NO: 3027), UUGguuugua (SEQ ID NO: 3028), UUGguuuguc (SEQ ID NO: 3029), UUUgcaagug (SEQ ID NO: 3030), UUUguaaaua (SEQ ID NO: 3031), UUUguaaaug (SEQ ID NO: 3032), UUUguaagaa (SEQ ID NO: 3033), UUUguaagac (SEQ ID NO: 3034), UUUguaagag (SEQ ID NO: 3035), UUUguaagca (SEQ ID NO: 3036), UUUguaaggu (SEQ ID NO: 3037), UUUguaagua (SEQ ID NO: 3038), UUUguaaguc (SEQ ID NO: 3039), UUUguaagug (SEQ ID NO: 3040), UUUguaaguu (SEQ ID NO: 3041), UUUguaauuu (SEQ ID NO: 3042), UUUguacagg (SEQ ID NO: 3043), UUUguacgug (SEQ ID NO: 3044), UUUguacuag (SEQ ID NO: 3045), UUUguacugu (SEQ ID NO: 3046), UUUguagguu (SEQ ID NO: 3047), UUUguauccu (SEQ ID NO: 3048), UUUguauguu (SEQ ID NO: 3049), UUUgugagca (SEQ ID NO: 3050), UUUgugagug (SEQ ID NO: 3051), UUUgugcguc (SEQ ID NO: 3052), UUUguguguc (SEQ ID NO: 3053), and uGGguaccug (SEQ ID NO: 3054). Additional exemplary gene sequences and splice site sequences (e.g., 5′ splice site sequences) include AAGgcaagau (SEQ ID NO: 96), AUGguaugug (SEQ ID NO: 937), GGGgugaggc (SEQ ID NO: 2281), CAGguaggug (SEQ ID NO: 1222), AAGgucagua (SEQ ID NO: 293), AAGguuagag (SEQ ID NO: 3055), AUGgcacuua (SEQ ID NO: 3056), UAAguaaguc (SEQ ID NO: 2423), UGGgugagcu (SEQ ID NO: 3057), CGAgcugggc (SEQ ID NO: 3058), AAAgcacccc (SEQ ID NO: 3059), UAGguggggg (SEQ ID NO: 3060), AGAguaacgu (SEQ ID NO: 3061), UCGgugaugu (SEQ ID NO: 3062), AAUgucaguu (SEQ ID NO: 516), AGGgucugag (SEQ ID NO: 3063), GAGgugacug (SEQ ID NO: 3064), AUGguagguu (SEQ ID NO: 3065), GAGgucuguc (SEQ ID NO: 2000), CAGguaugug (SEQ ID NO: 1260), CAAguacugc (SEQ ID NO: 3066), CACgugcgua (SEQ ID NO: 3067), CCGgugagcu (SEQ ID NO: 3068), CAGguacuuc (SEQ ID NO: 3069), CAGgcgagag (SEQ ID NO: 1115), GAAgcaagua (SEQ ID NO: 3070), AGGgugagca (SEQ ID NO: 789), CAGgcaaguc (SEQ ID NO: 3071), AAGgugaggc (SEQ ID NO: 344), CAGguaagua (SEQ ID NO: 1147), CCAguugggu (SEQ ID NO: 3072), AAGguguggg (SEQ ID NO: 3073), CAGguuggag (SEQ ID NO: 1484), CCGguaugaa (SEQ ID NO: 3074), UGGguaaugu (SEQ ID NO: 2845), CAGgugaggu (SEQ ID NO: 1344), AGAguaauag (SEQ ID NO: 3075), CAGguaugag (SEQ ID NO: 1249), AUGguaaguu (SEQ ID NO: 901), UUGguggguc (SEQ ID NO: 3015), UUUguaagca (SEQ ID NO: 3036), CUCguaugcc (SEQ ID NO: 3076), UAGguaagag (SEQ ID NO: 2483), UAGgcaaguu (SEQ ID NO: 3077), GGAguuaagu (SEQ ID NO: 3078), GAGguaugcc (SEQ ID NO: 1959), AAGguguggu (SEQ ID NO: 402), CAGgugggug (SEQ ID NO: 1415), UUAguaagua (SEQ ID NO: 2933), AAGguuggcu (SEQ ID NO: 3079), UGAguaugug (SEQ ID NO: 3080), CCAgccuucc (SEQ ID NO: 3081), CCUguacgug (SEQ ID NO: 3082), CCUguaggua (SEQ ID NO: 1601), CAGguacgcu (SEQ ID NO: 3083), GAGguucuuc (SEQ ID NO: 3084), AAGguugccu (SEQ ID NO: 3085), CGUguucacu (SEQ ID NO: 3086), CGGgugggga (SEQ ID NO: 3087), UAGgugggau (SEQ ID NO: 2614), CGGguaagga (SEQ ID NO: 3088), AAGguacuau (SEQ ID NO: 195), GGGguaagcu (SEQ ID NO: 2248), ACGguagagc (SEQ ID NO: 3089), CAGgugaaga (SEQ ID NO: 1318), GCGguaagag (SEQ ID NO: 3090), CAGguguugu (SEQ ID NO: 3091), GAAguuugug (SEQ ID NO: 3092), AUGgugagca (SEQ ID NO: 955), CGGguucgug (SEQ ID NO: 3093), AUUguccggc (SEQ ID NO: 3094), GAUgugugug (SEQ ID NO: 3095), AUGgucuguu (SEQ ID NO: 3096), AAGguaggau (SEQ ID NO: 219), CCGguaagau (SEQ ID NO: 1575), AAGguaaaga (SEQ ID NO: 126), GGGgugaguu (SEQ ID NO: 2285), AGGguuggug (SEQ ID NO: 808), GGAgugagug (SEQ ID NO: 2228), AGUguaagga (SEQ ID NO: 3097), UAGguaacug (SEQ ID NO: 2480), AAGgugaaga (SEQ ID NO: 3098), UGGguaagug (SEQ ID NO: 2843), CAGguaagag (SEQ ID NO: 1140), UAGgugagcg (SEQ ID NO: 3099), GAGguaaaaa (SEQ ID NO: 1865), GCCguaaguu (SEQ ID NO: 3100), AAGguuuugu (SEQ ID NO: 473), CAGgugagga (SEQ ID NO: 1341), ACAgcccaug (SEQ ID NO: 3101), GCGgugagcc (SEQ ID NO: 3102), CAGguaugca (SEQ ID NO: 1251), AUGguaccua (SEQ ID NO: 3103), CAAguaugua (SEQ ID NO: 1050), AUGgugguge (SEQ ID NO: 3104), UAAguggcag (SEQ ID NO: 3105), UAGguauagu (SEQ ID NO: 3106), CUGguauuua (SEQ ID NO: 3107), AGGguaaacg (SEQ ID NO: 3108), AUAguaagug (SEQ ID NO: 850), UUGguacuga (SEQ ID NO: 3109), GGUguaagcc (SEQ ID NO: 2303), GAGguggaua (SEQ ID NO: 3110), GAUguaagaa (SEQ ID NO: 3111), ACGgucaguu (SEQ ID NO: 3112), UAAguaaaca (SEQ ID NO: 3113), AAGguaucug (SEQ ID NO: 251), AGGguauuug (SEQ ID NO: 3114), AAGgugaaug (SEQ ID NO: 328), CUGgugaauu (SEQ ID NO: 1749), CAGguuuuuu (SEQ ID NO: 1514), CAUguaugug (SEQ ID NO: 1534), UUGguagagg (SEQ ID NO: 2973), AAGguaugcc (SEQ ID NO: 258), CAGgugccac (SEQ ID NO: 3115), UCGguauuga (SEQ ID NO: 2727), AAGguuugug (SEQ ID NO: 468), AAUguacagg (SEQ ID NO: 3116), CAUguggguu (SEQ ID NO: 1545), CAUgugaguu (SEQ ID NO: 1542), UUGguaaugu (SEQ ID NO: 2968), AGUguaggug (SEQ ID NO: 3117), GAGguaacuc (SEQ ID NO: 3118), GAGguggcgc (SEQ ID NO: 3119), CUGguaauug (SEQ ID NO: 3120), GAGguuugcu (SEQ ID NO: 3121), UGUguacgug (SEQ ID NO: 3122), UAGguaaaga (SEQ ID NO: 2468), CUAguaggca (SEQ ID NO: 3123), UCUgugaguc (SEQ ID NO: 2761), UCUguaaggc (SEQ ID NO: 3124), CAGguuugug (SEQ ID NO: 1509), GAGguagggc (SEQ ID NO: 1935), AAGguaacca (SEQ ID NO: 3125), ACUgugaguu (SEQ ID NO: 646), UAGguaauag (SEQ ID NO: 2495), AAAguaagcu (SEQ ID NO: 17), AUGgugagug (SEQ ID NO: 963), UAGguuugug (SEQ ID NO: 2645), AACguaggac (SEQ ID NO: 3126), GUAgcaggua (SEQ ID NO: 3127), GAGgucagac (SEQ ID NO: 3128), AGGguaugaa (SEQ ID NO: 3129), GAGguuagug (SEQ ID NO: 2089), CAGgcacgug (SEQ ID NO: 3130), GGGgcaagac (SEQ ID NO: 3131), CAGguguguc (SEQ ID NO: 1441), CAGguauuga (SEQ ID NO: 1265), CAGguauguc (SEQ ID NO: 1259), AAGgcaaggu (SEQ ID NO: 3132), UUGgugagaa (SEQ ID NO: 3133), AAGguaaaau (SEQ ID NO: 122), GGGguaagua (SEQ ID NO: 2251), AAGguaucuu (SEQ ID NO: 252), GACgugaguc (SEQ ID NO: 3134), UAUguaugcu (SEQ ID NO: 3135), AAGguacugu (SEQ ID NO: 199), CAGgugaacu (SEQ ID NO: 3136), CACguaaaug (SEQ ID NO: 3137), AAGgugugau (SEQ ID NO: 3138), GAAguauuug (SEQ ID NO: 3139), AAGgucugug (SEQ ID NO: 3140), AAGguggagg (SEQ ID NO: 3141), AAGguauaug (SEQ ID NO: 244), CAGguucuua (SEQ ID NO: 1477), AGGguaacca (SEQ ID NO: 730), CAGgugucac (SEQ ID NO: 1423), AAAguucugu (SEQ ID NO: 3142), UUGgugaguu (SEQ ID NO: 3007), CAAgugaguc (SEQ ID NO: 1067), UAGguagguc (SEQ ID NO: 2525), GCGgugagcu (SEQ ID NO: 2180), AUUgugagga (SEQ ID NO: 3143), CAGgugcaca (SEQ ID NO: 1361), CAGguuggaa (SEQ ID NO: 3144), CUGgucacuu (SEQ ID NO: 3145), GGAguaagug (SEQ ID NO: 2214), GAGgugggcu (SEQ ID NO: 2059), AAGguacuug (SEQ ID NO: 201), AGGguaggau (SEQ ID NO: 3146), AAUguguguu (SEQ ID NO: 3147), ACAguuaagu (SEQ ID NO: 568), GAGgugugug (SEQ ID NO: 2078), AAGgcgggcu (SEQ ID NO: 3148), AUAgcaagua (SEQ ID NO: 3149), AAGguuguua (SEQ ID NO: 454), CAAgcaaggc (SEQ ID NO: 3150), GUGguaauua (SEQ ID NO: 3151), UCUguucagu (SEQ ID NO: 3152), AGGguaggcc (SEQ ID NO: 754), AAGguaucau (SEQ ID NO: 3153), UAGguaccuu (SEQ ID NO: 2509), AAGguaugac (SEQ ID NO: 254), GGAguaggua (SEQ ID NO: 2219), UAAguuggca (SEQ ID NO: 3154), AGUgugagge (SEQ ID NO: 3155), GAGguuugug (SEQ ID NO: 3156), UGGgucugcu (SEQ ID NO: 3157), CAGgugaucc (SEQ ID NO: 1350), CAGgucagug (SEQ ID NO: 1283), AAGguaaggg (SEQ ID NO: 151), CAGgugcagu (SEQ ID NO: 3158), GAGguggguc (SEQ ID NO: 2064), GCUgugagug (SEQ ID NO: 2206), AAGguggagu (SEQ ID NO: 3159), GGGgucaguu (SEQ ID NO: 3160), AGCguaagug (SEQ ID NO: 719), AGAguaugaa (SEQ ID NO: 691), GGGguagggu (SEQ ID NO: 3161), AAGgccagca (SEQ ID NO: 3162), CGAguaugcc (SEQ ID NO: 3163), GUGgugageg (SEQ ID NO: 3164), AAUguaaauu (SEQ ID NO: 481), CAGgugcgca (SEQ ID NO: 1375), GGUguaugaa (SEQ ID NO: 3165), CUUgugaguu (SEQ ID NO: 1804), AAGguaucuc (SEQ ID NO: 250), AGAguaagga (SEQ ID NO: 665), UAGguaagac (SEQ ID NO: 2482), GAGgugagug (SEQ ID NO: 2026), CAGguguguu (SEQ ID NO: 1443), UUGgugagua (SEQ ID NO: 3004), AGGgcgaguu (SEQ ID NO: 3166), CAGguuuugc (SEQ ID NO: 3167), UUUgugaguu (SEQ ID NO: 3168), AGGguaagca (SEQ ID NO: 736), GAGguccucu (SEQ ID NO: 3169), CCAgcaggua (SEQ ID NO: 3170), GAGguucgcg (SEQ ID NO: 3171), CAGgugaucu (SEQ ID NO: 1351), ACUguaagua (SEQ ID NO: 625), AAGguaaauc (SEQ ID NO: 131), CAGgcaaaua (SEQ ID NO: 3172), GUGguaagca (SEQ ID NO: 2346), CAGguuaaau (SEQ ID NO: 3173), UUGguaauaa (SEQ ID NO: 3174), UAUguaggua (SEQ ID NO: 3175), CAGguaguau (SEQ ID NO: 1225), AAGgugugcc (SEQ ID NO: 3176), UGGguaagag (SEQ ID NO: 2834), CAGgcaagca (SEQ ID NO: 3177), UUGguaaggg (SEQ ID NO: 2961), AAGgcaggug (SEQ ID NO: 109), ACGguaaaug (SEQ ID NO: 3178), GCUgugagca (SEQ ID NO: 3179), AUGguacaca (SEQ ID NO: 3180), GUAguguguu (SEQ ID NO: 3181), ACUguaagag (SEQ ID NO: 3182), CCCgcagguc (SEQ ID NO: 3183), GAGgugagcc (SEQ ID NO: 2019), GAGgugcugu (SEQ ID NO: 3184), UAAguaugcu (SEQ ID NO: 3185), GAGgccaucu (SEQ ID NO: 3186), UCAgugagug (SEQ ID NO: 2700), CAGgugcuac (SEQ ID NO: 3187), AAUgugggug (SEQ ID NO: 533), GAGgugugaa (SEQ ID NO: 3188), CUGguagguc (SEQ ID NO: 1730), GUGgcgcgcg (SEQ ID NO: 3189), CAGgugcaaa (SEQ ID NO: 1359), UAAguggagg (SEQ ID NO: 3190), CAUgugggua (SEQ ID NO: 3191), GAGguagggu (SEQ ID NO: 3192), AAAgugaguu (SEQ ID NO: 61), AGGguucuag (SEQ ID NO: 3193), UGUgugagcu (SEQ ID NO: 3194), AGGgugaauc (SEQ ID NO: 3195), CAGgucaggg (SEQ ID NO: 3196), AAGgucccug (SEQ ID NO: 3197), CUGguagagu (SEQ ID NO: 3198), UAGgucaguu (SEQ ID NO: 2570), AAAguaaggg (SEQ ID NO: 19), CAAguaugug (SEQ ID NO: 1052), CAGgugcuuu (SEQ ID NO: 3199), AAGguaauuc (SEQ ID NO: 169), GGGgugcacg (SEQ ID NO: 3200), ACUgugcuac (SEQ ID NO: 3201), CAGguaccua (SEQ ID NO: 3202), CAGguagcuu (SEQ ID NO: 1211), UGGgugaggc (SEQ ID NO: 2873), CUGguacauu (SEQ ID NO: 1718), AGGguaaucu (SEQ ID NO: 3203), CAGguacaag (SEQ ID NO: 1161), CAGguaauuc (SEQ ID NO: 1157), AGGgcacuug (SEQ ID NO: 3204), UAGgugagaa (SEQ ID NO: 2587), GAGguaaugc (SEQ ID NO: 3205), CCAgugaguu (SEQ ID NO: 3206), AAAguaugug (SEQ ID NO: 44), CUGgugaauc (SEQ ID NO: 3207), UAUguaugua (SEQ ID NO: 2663), CCUgcaggug (SEQ ID NO: 3208), CAGguaucug (SEQ ID NO: 1245), GAGgugaggu (SEQ ID NO: 3209), CUGguaaaac (SEQ ID NO: 3210), UGUgugugcu (SEQ ID NO: 3211), CAGguuaagu (SEQ ID NO: 3212), CAGguaaucc (SEQ ID NO: 1152), UAGguauuug (SEQ ID NO: 3213), UGGguagguc (SEQ ID NO: 2852), CAGguaacag (SEQ ID NO: 1129), AGCgugcgug (SEQ ID NO: 3214), AAGgucagga (SEQ ID NO: 289), GGUgugagcc (SEQ ID NO: 2312), CUGguaagua (SEQ ID NO: 1707), GGGgugggca (SEQ ID NO: 3215), AAGgugggaa (SEQ ID NO: 376), CAGgugagug (SEQ ID NO: 1347), CUGguuguua (SEQ ID NO: 3216), CAGguaauag (SEQ ID NO: 3217), UAGgugaguu (SEQ ID NO: 3218), AGAguaaguu (SEQ ID NO: 671), UAGguaaucc (SEQ ID NO: 3219), CCGgugacug (SEQ ID NO: 3220), GUCgugauua (SEQ ID NO: 3221), CUUguaagug (SEQ ID NO: 1794), UAGguaguca (SEQ ID NO: 3222), CUGguaaguc (SEQ ID NO: 3223), AGGgugagcg (SEQ ID NO: 3224), CAGguaugga (SEQ ID NO: 1255), AUUgugacca (SEQ ID NO: 3225), GUUgugggua (SEQ ID NO: 2411), AAGguacaag (SEQ ID NO: 173), CUAgcaagug (SEQ ID NO: 3226), CUGgugagau (SEQ ID NO: 3227), CAGgugggca (SEQ ID NO: 1406), AUGgcucgag (SEQ ID NO: 3228), CUGguacguu (SEQ ID NO: 1720), UUGgugugua (SEQ ID NO: 3229), GAGgugucug (SEQ ID NO: 3230), GAGgugggac (SEQ ID NO: 3231), GGGgugggag (SEQ ID NO: 3232), GCAgcgugag (SEQ ID NO: 3233), GAGguaaaga (SEQ ID NO: 1870), GAGguaugua (SEQ ID NO: 1965), AAGgugagac (SEQ ID NO: 336), AAGguacaau (SEQ ID NO: 174), CUGguaugag (SEQ ID NO: 3234), AACguaaaau (SEQ ID NO: 3235), GUGguaggga (SEQ ID NO: 2364), CUGguaugug (SEQ ID NO: 1737), CUUguaagca (SEQ ID NO: 3236), AAGguaggga (SEQ ID NO: 223), AUUguaagcc (SEQ ID NO: 3237), AUGguaagcu (SEQ ID NO: 895), CAGgugaauu (SEQ ID NO: 1322), UAGgugaaua (SEQ ID NO: 2581), CAAguaugga (SEQ ID NO: 3238), AUGguauggc (SEQ ID NO: 936), GAGgucaugc (SEQ ID NO: 3239), CAGguacccu (SEQ ID NO: 1174), ACAgugagac (SEQ ID NO: 3240), CAGgucugau (SEQ ID NO: 3241), GAAguugggu (SEQ ID NO: 3242), CUGgugegug (SEQ ID NO: 1767), CAGguacgag (SEQ ID NO: 1180), ACAgugagcc (SEQ ID NO: 556), AAGguaagua (SEQ ID NO: 153), GGAguaaggc (SEQ ID NO: 3243), GAGgugugua (SEQ ID NO: 2077), AAGgucauuu (SEQ ID NO: 3244), CAGguagucu (SEQ ID NO: 3245), AUGguaucug (SEQ ID NO: 3246), AAGguaaacu (SEQ ID NO: 125), GAGguaggug (SEQ ID NO: 1938), CUGguaagca (SEQ ID NO: 1700), AGGguaagag (SEQ ID NO: 734), AAAguaaagc (SEQ ID NO: 3247), CAGguuugag (SEQ ID NO: 1502), GAGgcgggua (SEQ ID NO: 3248), CGAguacgau (SEQ ID NO: 3249), CAGguuguug (SEQ ID NO: 1495), AAAguauggg (SEQ ID NO: 3250), UAGgcugguc (SEQ ID NO: 3251), AAGguaagga (SEQ ID NO: 149), AAGguuuccu (SEQ ID NO: 458), UUGguaaaac (SEQ ID NO: 3252), GAGguaagua (SEQ ID NO: 1893), CAGguucaag (SEQ ID NO: 1465), UGGguuaugu (SEQ ID NO: 3253), GAGgugaguu (SEQ ID NO: 2027), ACGgugaaac (SEQ ID NO: 598), GAUguaacca (SEQ ID NO: 3254), AAGgugcggg (SEQ ID NO: 3255), CCGguacgug (SEQ ID NO: 3256), GAUgugagaa (SEQ ID NO: 3257), GUGgegguga (SEQ ID NO: 3258), CAGguauuag (SEQ ID NO: 3259), GAGguuggga (SEQ ID NO: 3260), AAGgcuagua (SEQ ID NO: 3261), AAGgugggcg (SEQ ID NO: 381), CAGgcaggga (SEQ ID NO: 3262), AAUguuaguu (SEQ ID NO: 3263), GAGguaaagg (SEQ ID NO: 3264), CAGgugugcu (SEQ ID NO: 1437), CUGguaugau (SEQ ID NO: 1733), AUGguuaguc (SEQ ID NO: 978), CUGgugagaa (SEQ ID NO: 1751), CAGgccggcg (SEQ ID NO: 3265), CAGgugacug (SEQ ID NO: 1332), AAAguaaggu (SEQ ID NO: 20), UAAguacuug (SEQ ID NO: 3266), AAGguaaagc (SEQ ID NO: 127), UCGguagggg (SEQ ID NO: 3267), CAGguaggaa (SEQ ID NO: 1212), AGUguaagca (SEQ ID NO: 817), CCCgugagau (SEQ ID NO: 3268), GUGguuguuu (SEQ ID NO: 3269), CAGguuugcc (SEQ ID NO: 1504), AGGguauggg (SEQ ID NO: 766), UAAguaagug (SEQ ID NO: 2424), GAGguaagac (SEQ ID NO: 3270), GAUguagguc (SEQ ID NO: 3271), CAAguaggug (SEQ ID NO: 1043), AUAguaaaua (SEQ ID NO: 845), GAGguugggg (SEQ ID NO: 3272), GAGgcgagua (SEQ ID NO: 3273), CAGguagugu (SEQ ID NO: 1229), GUGguaggug (SEQ ID NO: 2366), CAAgugagug (SEQ ID NO: 1068), AAGgugacaa (SEQ ID NO: 330), CCAgcguaau (SEQ ID NO: 3274), ACGgugaggu (SEQ ID NO: 3275), GGGguauauu (SEQ ID NO: 3276), CAGgugagua (SEQ ID NO: 1345), AAGgugcgug (SEQ ID NO: 364), UAUguaaauu (SEQ ID NO: 3277), CAGgucagua (SEQ ID NO: 1281), ACGguacuua (SEQ ID NO: 3278), GAGgucagca (SEQ ID NO: 3279), UAAguaugua (SEQ ID NO: 2431), GGGgucagac (SEQ ID NO: 3280), AAUgugugag (SEQ ID NO: 3281), UCCgucagua (SEQ ID NO: 3282), CAGgugcuuc (SEQ ID NO: 1391), CCAguuagug (SEQ ID NO: 3283), CCGgugggcg (SEQ ID NO: 1590), AGGgugcaug (SEQ ID NO: 3284), GGGguaggau (SEQ ID NO: 3285), UAGgugggcc (SEQ ID NO: 2615), GAGguguucg (SEQ ID NO: 3286), UUGgcaagaa (SEQ ID NO: 3287), UCCguaagua (SEQ ID NO: 3288), CAGguguaag (SEQ ID NO: 3289), CUCgugagua (SEQ ID NO: 1680), GAGguguuuu (SEQ ID NO: 3290), GAGgugagca (SEQ ID NO: 2018), GAGguaaagu (SEQ ID NO: 1872), AAGguacguu (SEQ ID NO: 193), CAGguccagu (SEQ ID NO: 1291), AUGgugaaac (SEQ ID NO: 947), GUAgugagcu (SEQ ID NO: 3291), CAGgugaaaa (SEQ ID NO: 3292), AGGguacagg (SEQ ID NO: 3293), AAGguaacgc (SEQ ID NO: 3294), AAGguauacc (SEQ ID NO: 3295), CCUgugagau (SEQ ID NO: 3296), GGGguacgug (SEQ ID NO: 3297), GAGguauggu (SEQ ID NO: 1964), UAGguauuau (SEQ ID NO: 2557), GAAguaggag (SEQ ID NO: 3298), UCGguaaggg (SEQ ID NO: 3299), CCGguaagcg (SEQ ID NO: 3300), GAAguaauua (SEQ ID NO: 1823), CAGgugaguc (SEQ ID NO: 1346), AAGgucaaga (SEQ ID NO: 279), AUGguaaguc (SEQ ID NO: 899), CAGgugagcu (SEQ ID NO: 1340), CCAguuuuug (SEQ ID NO: 3301), CAGgugggag (SEQ ID NO: 1404), AAGguauuau (SEQ ID NO: 270), AAGguaaaua (SEQ ID NO: 130), AAGgugcugu (SEQ ID NO: 3302), AAAguacacc (SEQ ID NO: 3303), CUGguucgug (SEQ ID NO: 1783), UCAguaaguc (SEQ ID NO: 2690), GAAguacgug (SEQ ID NO: 3304), CAGgugacaa (SEQ ID NO: 1323), UGGguaagaa (SEQ ID NO: 2832), UGUguagggg (SEQ ID NO: 3305), GAGguaggca (SEQ ID NO: 1932), UUGgugaggc (SEQ ID NO: 3306), AUGgugugua (SEQ ID NO: 974), CAGguccucc (SEQ ID NO: 3307), UUGguaaaug (SEQ ID NO: 2953), GCUgugaguu (SEQ ID NO: 2207), AUGgucugua (SEQ ID NO: 3308), CAUgcaggug (SEQ ID NO: 3309), CUGguacace (SEQ ID NO: 3310), CAGguccuua (SEQ ID NO: 3311), CAAguaaucu (SEQ ID NO: 1031), AUGgcagccu (SEQ ID NO: 3312), AAGgucagaa (SEQ ID NO: 282), AACgugaggc (SEQ ID NO: 3313), CAGgcacgca (SEQ ID NO: 1106), ACGguccagg (SEQ ID NO: 3314), UCUguacaua (SEQ ID NO: 3315), GAGgugauua (SEQ ID NO: 3316), ACGguaaaua (SEQ ID NO: 3317), AUGguaacug (SEQ ID NO: 3318), CAGgcgcguu (SEQ ID NO: 3319), CAGguauaga (SEQ ID NO: 1235), AAGguuuguu (SEQ ID NO: 3320), CAGguaugaa (SEQ ID NO: 1247), UAGguuggua (SEQ ID NO: 2638), CUGgugagac (SEQ ID NO: 1752), CAGguuagga (SEQ ID NO: 3321), AUGgugacug (SEQ ID NO: 3322), UUGguauccc (SEQ ID NO: 3323), CUUguaggac (SEQ ID NO: 3324), AAAguguguu (SEQ ID NO: 69), CAGguuucuu (SEQ ID NO: 1500), GGGguauggc (SEQ ID NO: 3325), GGGguaggac (SEQ ID NO: 3326), ACUguaaguc (SEQ ID NO: 626), AUCguaagcu (SEQ ID NO: 3327), UAGguucccc (SEQ ID NO: 2636), GGUgugagca (SEQ ID NO: 3328), CUGguuggua (SEQ ID NO: 3329), GGGguuaggg (SEQ ID NO: 3330), UGAguaagaa (SEQ ID NO: 3331), GAGguauucc (SEQ ID NO: 1969), UGGguuaguc (SEQ ID NO: 2893), CAGgcucgug (SEQ ID NO: 3332), UAGguagagu (SEQ ID NO: 3333), UAGgugcccu (SEQ ID NO: 3334), AAAgugagua (SEQ ID NO: 58), GAGguucaua (SEQ ID NO: 2094), UUGguaagag (SEQ ID NO: 2958), ACCgugugua (SEQ ID NO: 3335), UAUguaguau (SEQ ID NO: 3336), UGGguaauag (SEQ ID NO: 3337), CAGgucugaa (SEQ ID NO: 3338), AAAguauaaa (SEQ ID NO: 3339), GUGgugaguc (SEQ ID NO: 3340), AGUgugauua (SEQ ID NO: 3341), UUGgugugug (SEQ ID NO: 3020), CAGgugaugg (SEQ ID NO: 1353), GCUgugagua (SEQ ID NO: 2204), CAGguacaug (SEQ ID NO: 1169), AAGguacagu (SEQ ID NO: 178), GAAguuguag (SEQ ID NO: 3342), CAGgugauua (SEQ ID NO: 1355), UAGgugaauu (SEQ ID NO: 2583), GGUguuaaua (SEQ ID NO: 3343), CAGguauuua (SEQ ID NO: 1268), CAAguacucg (SEQ ID NO: 3344), CAAguaagaa (SEQ ID NO: 1022), AAGguaccuu (SEQ ID NO: 188), ACGgugaggg (SEQ ID NO: 3345), UGAgcaggca (SEQ ID NO: 3346), GGGgugaccg (SEQ ID NO: 3347), GAGguaaaug (SEQ ID NO: 1875), CGGguuugug (SEQ ID NO: 3348), AAGgugagcg (SEQ ID NO: 341), GUGguaugga (SEQ ID NO: 3349), CUGguaagga (SEQ ID NO: 1703), GAGguaccag (SEQ ID NO: 1911), CCGgugagug (SEQ ID NO: 1587), AAGguuagaa (SEQ ID NO: 416), GAGguacuug (SEQ ID NO: 1921), AGAguaaaac (SEQ ID NO: 651), UCUgugagua (SEQ ID NO: 2760), AAGgcgggaa (SEQ ID NO: 3350), CAGguaugcg (SEQ ID NO: 1253), AGGguaaaac (SEQ ID NO: 3351), AAGgugacug (SEQ ID NO: 333), AGGguauguu (SEQ ID NO: 3352), AAGguaugua (SEQ ID NO: 263), CAGgucucuc (SEQ ID NO: 1302), CAGgcaugua (SEQ ID NO: 3353), CUGguaggua (SEQ ID NO: 1729), AAGgucaugc (SEQ ID NO: 3354), CAGguacaca (SEQ ID NO: 1163), GAUguacguu (SEQ ID NO: 3355), ACAguacgug (SEQ ID NO: 3356), ACGguaccca (SEQ ID NO: 3357), CAGguagugc (SEQ ID NO: 3358), ACAguaagag (SEQ ID NO: 3359), GGUgcacacc (SEQ ID NO: 3360), GAGguguaac (SEQ ID NO: 3361), AAGgugugua (SEQ ID NO: 403), UAGguacuua (SEQ ID NO: 3362), GCGguacugc (SEQ ID NO: 3363), UGGguaaguc (SEQ ID NO: 2842), CAUguaggua (SEQ ID NO: 1529), CAGguaggau (SEQ ID NO: 3364), CAGgucuggc (SEQ ID NO: 3365), GUGguuuuaa (SEQ ID NO: 3366), CAGgugggaa (SEQ ID NO: 1402), UGGgugagua (SEQ ID NO: 2875), CGAgugagcc (SEQ ID NO: 3367), AAGguauggc (SEQ ID NO: 261), AGUguuguca (SEQ ID NO: 3368), CAGgugauuu (SEQ ID NO: 1358), UAGguaucuc (SEQ ID NO: 2544), UAAguauguu (SEQ ID NO: 3369), AAGguugagc (SEQ ID NO: 3370), AGAguaaaga (SEQ ID NO: 653), GGUguaagua (SEQ ID NO: 3371), GGGgugagcu (SEQ ID NO: 2279), CAGguauaau (SEQ ID NO: 3372), GAGguacaaa (SEQ ID NO: 1904), AUGguaccaa (SEQ ID NO: 3373), UAGguagggg (SEQ ID NO: 2523), UGAgucagaa (SEQ ID NO: 3374), AAGgcaauua (SEQ ID NO: 3375), UUGguaagau (SEQ ID NO: 3376), CAGguacaga (SEQ ID NO: 1165), AGAguuagag (SEQ ID NO: 3377), CAGgugcguc (SEQ ID NO: 1381), GAGguauuac (SEQ ID NO: 3378), ACGguacaga (SEQ ID NO: 3379), CAGgucuucc (SEQ ID NO: 1313), AAGguaaggu (SEQ ID NO: 152), GAGguaauuu (SEQ ID NO: 1903), AGUguaggcu (SEQ ID NO: 3380), AAAguaagcg (SEQ ID NO: 3381), CCUguaagcc (SEQ ID NO: 3382), AGGgugauuu (SEQ ID NO: 3383), UGUguaugaa (SEQ ID NO: 3384), CUGguacaca (SEQ ID NO: 3385), AGGguagaga (SEQ ID NO: 3386), AUAguaagca (SEQ ID NO: 848), AGAguaugua (SEQ ID NO: 3387), UUGgucagca (SEQ ID NO: 3388), CAGgcaaguu (SEQ ID NO: 1105), AAGguauaua (SEQ ID NO: 242), AAGgucugga (SEQ ID NO: 314), CAGguacgca (SEQ ID NO: 1181), AGGgugcggg (SEQ ID NO: 3389), AUGguaagug (SEQ ID NO: 900), AAAgugauga (SEQ ID NO: 3390), UGCgugagua (SEQ ID NO: 3391), AGAguaggga (SEQ ID NO: 684), UGUguaggua (SEQ ID NO: 2912), UAGguaggau (SEQ ID NO: 2521), UAAgugagug (SEQ ID NO: 2440), GCUguaagua (SEQ ID NO: 2193), GAAguaagaa (SEQ ID NO: 1814), UCGgugaggc (SEQ ID NO: 2733), UAGguauuuu (SEQ ID NO: 2564), AAGguacaca (SEQ ID NO: 3392), AAGguaggua (SEQ ID NO: 227), UGGguagguu (SEQ ID NO: 2854), ACAgcaagua (SEQ ID NO: 541), GAGguaggag (SEQ ID NO: 1931), UGGgugaguu (SEQ ID NO: 2878), GCGgugagau (SEQ ID NO: 3393), CCUguagguu (SEQ ID NO: 3394), CAGgugugua (SEQ ID NO: 1440), CUGguaagcc (SEQ ID NO: 1701), AAGgugauuc (SEQ ID NO: 3395), CAGguagcua (SEQ ID NO: 1208), GUUguaagug (SEQ ID NO: 3396), AUGguaagca (SEQ ID NO: 893), AUAguaggga (SEQ ID NO: 3397), GGGguucgcu (SEQ ID NO: 3398), CCGgucagag (SEQ ID NO: 3399), GUAguaugag (SEQ ID NO: 3400), CGUguaagau (SEQ ID NO: 3401), UGAguaggca (SEQ ID NO: 3402), UCAguaugua (SEQ ID NO: 3403), GAGguaucug (SEQ ID NO: 1954), AGAguauuuu (SEQ ID NO: 3404), AAGguuguag (SEQ ID NO: 3405), AGUguaaguu (SEQ ID NO: 821), CGGguaaguu (SEQ ID NO: 1626), UCGgugcgga (SEQ ID NO: 3406), UAGguaagua (SEQ ID NO: 2491), GAAguuagau (SEQ ID NO: 3407), GCUgugagac (SEQ ID NO: 3408), CAGgcaggua (SEQ ID NO: 3409), CAGguagggg (SEQ ID NO: 1218), UAAguuaaga (SEQ ID NO: 3410), AUGguggguu (SEQ ID NO: 970), UAGguaaguu (SEQ ID NO: 2494), CUGguaaauu (SEQ ID NO: 1690), CCGguaagga (SEQ ID NO: 1577), GAGgcaggca (SEQ ID NO: 3411), CAUguaagug (SEQ ID NO: 1523), AAGgugccua (SEQ ID NO: 3412), UUGguaggga (SEQ ID NO: 2977), AAGguaaaca (SEQ ID NO: 123), CGGgugugag (SEQ ID NO: 3413), GGGgugugag (SEQ ID NO: 3414), UCCguggguc (SEQ ID NO: 3415), ACGguaaauc (SEQ ID NO: 3416), UCAguaggua (SEQ ID NO: 3417), CAGgucagcc (SEQ ID NO: 1278), CAGgcggugg (SEQ ID NO: 3418), CGAguaagcu (SEQ ID NO: 3419), CCCgugagca (SEQ ID NO: 3420), AAAguaauga (SEQ ID NO: 3421), CUGguaagcu (SEQ ID NO: 1702), CGGguaacca (SEQ ID NO: 3422), CAGgucgcac (SEQ ID NO: 3423), GAGguaggcc (SEQ ID NO: 3424), UAGgugagcc (SEQ ID NO: 2591), UAGguaggca (SEQ ID NO: 3425), GCGgugcgug (SEQ ID NO: 3426), AUGgugagua (SEQ ID NO: 961), GGGgugaggg (SEQ ID NO: 2282), GAGgucacac (SEQ ID NO: 3427), CAGguaggcc (SEQ ID NO: 3428), CAAgugcuga (SEQ ID NO: 3429), GUCgucuuca (SEQ ID NO: 3430), CAUguaagaa (SEQ ID NO: 1518), GUAguaagga (SEQ ID NO: 3431), UAGguuugua (SEQ ID NO: 2643), CAAguuagag (SEQ ID NO: 3432), AAGguagagu (SEQ ID NO: 208), AAGgugagau (SEQ ID NO: 338), AAAguaggua (SEQ ID NO: 37), ACAgugaauc (SEQ ID NO: 3433), CAGgugugcg (SEQ ID NO: 1436), CAGgucggcc (SEQ ID NO: 1299), AAGguaguau (SEQ ID NO: 3434), ACUgucaguc (SEQ ID NO: 3435), UCUgcagccu (SEQ ID NO: 3436), CGAguaagug (SEQ ID NO: 3437), AGAguaauua (SEQ ID NO: 3438), AGUgugagug (SEQ ID NO: 837), CCGgugagcg (SEQ ID NO: 3439), AAGguaaccu (SEQ ID NO: 3440), AAGguugugg (SEQ ID NO: 3441), AAGgcauggg (SEQ ID NO: 3442), AAGgucagag (SEQ ID NO: 284), ACGguaaggu (SEQ ID NO: 3443), GGGgugagca (SEQ ID NO: 3444), GAGguugcuu (SEQ ID NO: 3445), AAGguaucgc (SEQ ID NO: 3446), CCGguaaagg (SEQ ID NO: 3447), AAAguuaaug (SEQ ID NO: 3448), UAGguacgag (SEQ ID NO: 2510), ACCguaauua (SEQ ID NO: 3449), GGGguaagga (SEQ ID NO: 2249), CCGguaacgc (SEQ ID NO: 3450), CAGgucagaa (SEQ ID NO: 1275), AAGguacuga (SEQ ID NO: 197), GAGgugacca (SEQ ID NO: 2010), GGGgugagcc (SEQ ID NO: 2277), AAGguacagg (SEQ ID NO: 177), AUGguaauua (SEQ ID NO: 3451), CAGgugagag (SEQ ID NO: 1335), AAGgugacuc (SEQ ID NO: 3452), AUAguaagua (SEQ ID NO: 849), GAGguaaacc (SEQ ID NO: 1869), CAGgugggau (SEQ ID NO: 1405), CAGgugagaa (SEQ ID NO: 1333), AGGguaaaaa (SEQ ID NO: 3453), GAGgugugac (SEQ ID NO: 3454), CACguaagcu (SEQ ID NO: 3455), CAGguccccc (SEQ ID NO: 3456), CAGgucaggu (SEQ ID NO: 3457), CGGguaaguc (SEQ ID NO: 3458), ACGguauggg (SEQ ID NO: 3459), GAUguaaguu (SEQ ID NO: 2123), CAAguaauau (SEQ ID NO: 3460), CAGguugggg (SEQ ID NO: 3461), CCUgugcugg (SEQ ID NO: 3462), AAGguaugau (SEQ ID NO: 256), AGGguagagg (SEQ ID NO: 3463), AAGguggguu (SEQ ID NO: 386), CAGgugugaa (SEQ ID NO: 1430), UUGguaugug (SEQ ID NO: 2988), UUGguaucuc (SEQ ID NO: 2985), GGGgugagug (SEQ ID NO: 2284), CUGgugugug (SEQ ID NO: 1779), AGGguagggc (SEQ ID NO: 3464), GUGgugagua (SEQ ID NO: 3465), CAGguaugua (SEQ ID NO: 1258), AAGguacauu (SEQ ID NO: 181), UUAguaagug (SEQ ID NO: 2934), AAUguauauc (SEQ ID NO: 3466), CUUguaagua (SEQ ID NO: 1793), GAGguuagua (SEQ ID NO: 2087), CAGguaaggu (SEQ ID NO: 1146), CAGguaaugu (SEQ ID NO: 1155), AGGgugaggc (SEQ ID NO: 3467), CAGguauuuc (SEQ ID NO: 1269), CAGgucugga (SEQ ID NO: 1307), GGGgugugcu (SEQ ID NO: 3468), UAGgugagug (SEQ ID NO: 2598), AAUguaaccu (SEQ ID NO: 3469), UAAgugaguc (SEQ ID NO: 2439), CAGgugcacu (SEQ ID NO: 3470), ACGguaagua (SEQ ID NO: 579), GAGguauccu (SEQ ID NO: 3471), UCUguaaguc (SEQ ID NO: 2745), CAGguauuca (SEQ ID NO: 1263), UGUguaagug (SEQ ID NO: 3472), CCAgcaaggc (SEQ ID NO: 3473), GAGgugaagg (SEQ ID NO: 2006), AAUguggggu (SEQ ID NO: 3474), UCGgugcgug (SEQ ID NO: 3475), UUGguaaggc (SEQ ID NO: 3476), GAGguaagug (SEQ ID NO: 3477), AAAguaagau (SEQ ID NO: 14), UAGgucuuuu (SEQ ID NO: 3478), GAGgucugau (SEQ ID NO: 3479), CCAguuagag (SEQ ID NO: 3480), UGGgugaaaa (SEQ ID NO: 3481), AGAguaagau (SEQ ID NO: 662), CAGguaauug (SEQ ID NO: 1158), CAGgccgguc (SEQ ID NO: 3482), CCGguaagag (SEQ ID NO: 3483), GAGgugagcu (SEQ ID NO: 2021), CUGguaagac (SEQ ID NO: 3484), CAGgugagau (SEQ ID NO: 1336), CUGguuuguu (SEQ ID NO: 3485), UGGguaggua (SEQ ID NO: 3486), CAGguuagug (SEQ ID NO: 1457), CAGguguucg (SEQ ID NO: 3487), CGGguagguc (SEQ ID NO: 3488), GUGguacaua (SEQ ID NO: 3489), AAGguacuaa (SEQ ID NO: 194), GAUgugagua (SEQ ID NO: 3490), UGUguaagac (SEQ ID NO: 2904), GAGguagccg (SEQ ID NO: 3491), UAGgugaucu (SEQ ID NO: 3492), CAGguacgug (SEQ ID NO: 1185), CUUgucaguc (SEQ ID NO: 3493), GAGguaucac (SEQ ID NO: 3494), GAGguaauga (SEQ ID NO: 3495), AAGguaacac (SEQ ID NO: 3496), CAGguaaagc (SEQ ID NO: 1123), AAGgcaagua (SEQ ID NO: 3497), CGCgugagcc (SEQ ID NO: 3498), AGUgugcguu (SEQ ID NO: 3499), GAUguaagca (SEQ ID NO: 2118), AAGguaauag (SEQ ID NO: 159), GGAgcaguug (SEQ ID NO: 3500), AGCguaagau (SEQ ID NO: 3501), AAGgucaggc (SEQ ID NO: 290), GAGguauuca (SEQ ID NO: 3502), AAUguaaagu (SEQ ID NO: 3503), CAGguaacaa (SEQ ID NO: 3504), UCGguaggug (SEQ ID NO: 3505), AAAguaaguc (SEQ ID NO: 22), CGGgugcagu (SEQ ID NO: 3506), GGUgugugca (SEQ ID NO: 3507), UGAgugagaa (SEQ ID NO: 2794), CACguguaag (SEQ ID NO: 3508), GUGguuggua (SEQ ID NO: 3509), GCAgccuuga (SEQ ID NO: 3510), CGAgugugau (SEQ ID NO: 3511), CAGguauaua (SEQ ID NO: 3512), UAUguaugug (SEQ ID NO: 2665), CCCgugguca (SEQ ID NO: 3513), AUGguaagac (SEQ ID NO: 890), GAGgugugga (SEQ ID NO: 2074), AGUguauccu (SEQ ID NO: 3514), UGAguguguc (SEQ ID NO: 3515), UGGguaaucu (SEQ ID NO: 3516), AUGgcagguu (SEQ ID NO: 3517), GAGguaagau (SEQ ID NO: 1884), UCAgcagcgu (SEQ ID NO: 3518), AAGgugggau (SEQ ID NO: 378), CGGgugcgcu (SEQ ID NO: 3519), CAGgugucug (SEQ ID NO: 1429), AGCgugguaa (SEQ ID NO: 3520), AAUgugaaug (SEQ ID NO: 3521), UCGgugagac (SEQ ID NO: 3522), UAGguaaagc (SEQ ID NO: 3523), CUGguaaaag (SEQ ID NO: 3524), CCGgugcgga (SEQ ID NO: 3525), CAGguacuca (SEQ ID NO: 3526), CAGguagcaa (SEQ ID NO: 1203), GAAguugagu (SEQ ID NO: 3527), GAGguggagg (SEQ ID NO: 2052), AGGguaugag (SEQ ID NO: 762), UAGguaugcu (SEQ ID NO: 3528), UAGgugagac (SEQ ID NO: 2588), CAGguaauua (SEQ ID NO: 1156), CGUguaagcc (SEQ ID NO: 3529), CUUguaaguu (SEQ ID NO: 1795), AAGguaacuu (SEQ ID NO: 140), UCGgcaaggc (SEQ ID NO: 3530), GAGguucucg (SEQ ID NO: 3531), GAGgugggcg (SEQ ID NO: 2058), AAGgcaugug (SEQ ID NO: 3532), CUGguauguu (SEQ ID NO: 1738), UAAgucauuu (SEQ ID NO: 3533), CAUguaauua (SEQ ID NO: 1525), AAUguaaaga (SEQ ID NO: 3534), UAGgugcuca (SEQ ID NO: 3535), AAGguaaugg (SEQ ID NO: 166), GAGguacuga (SEQ ID NO: 3536), UGGguaagua (SEQ ID NO: 2841), UGGguaaaaa (SEQ ID NO: 3537), AAGgugagcu (SEQ ID NO: 342), UACgugaguu (SEQ ID NO: 3538), AGGgugagcc (SEQ ID NO: 790), CGGgugagga (SEQ ID NO: 3539), UGGgugagag (SEQ ID NO: 2869), GGUguaagcu (SEQ ID NO: 3540), CGGguggguu (SEQ ID NO: 1648), CCAgcuaagu (SEQ ID NO: 3541), AAGguuuguc (SEQ ID NO: 467), GAGguuagac (SEQ ID NO: 2084), GAGguaccuc (SEQ ID NO: 3542), UUUguaaguu (SEQ ID NO: 3041), GAGguuagga (SEQ ID NO: 3543), CAGguaggga (SEQ ID NO: 1216), AGGguaauac (SEQ ID NO: 744), UGCgugugua (SEQ ID NO: 3544), CCAguaacca (SEQ ID NO: 3545), AGGgucuguc (SEQ ID NO: 3546), UGGguaugua (SEQ ID NO: 2860), GUGguaagcu (SEQ ID NO: 2348), CAGguaaccu (SEQ ID NO: 3547), AAGgugaguu (SEQ ID NO: 350), UAGguucgug (SEQ ID NO: 3548), AAAguuagua (SEQ ID NO: 3549), UGGgcaaguc (SEQ ID NO: 2816), AAGgcacagu (SEQ ID NO: 3550), GUUguaaguc (SEQ ID NO: 2401), AAGguuugcc (SEQ ID NO: 462), CUUgcauggg (SEQ ID NO: 3551), GCGgugagua (SEQ ID NO: 3552), GGGguaagcg (SEQ ID NO: 3553), GCCguaagaa (SEQ ID NO: 3554), GAGgucggga (SEQ ID NO: 3555), UUGguauugu (SEQ ID NO: 2990), AGUgugagac (SEQ ID NO: 3556), CUGgugggga (SEQ ID NO: 1770), AGAguaaggu (SEQ ID NO: 668), CCGguggguc (SEQ ID NO: 3557), CAGguauucu (SEQ ID NO: 1264), UGGguaacgu (SEQ ID NO: 3558), UUGgugagag (SEQ ID NO: 3559), UAGguacccu (SEQ ID NO: 3560), GGGgugcguc (SEQ ID NO: 3561), AAGgcaggag (SEQ ID NO: 3562), ACGguacauu (SEQ ID NO: 3563), GAGguaguua (SEQ ID NO: 1946), CAGguauggg (SEQ ID NO: 1256), UUUguguguc (SEQ ID NO: 3053), CAGguacuua (SEQ ID NO: 1194), AUGguauacu (SEQ ID NO: 3564), AGUgugagcc (SEQ ID NO: 833), ACAguaacga (SEQ ID NO: 3565), CUGguaccca (SEQ ID NO: 3566), CAGguaaccc (SEQ ID NO: 3567), GGAguaagua (SEQ ID NO: 3568), GAGgugggug (SEQ ID NO: 2065), ACUguauguc (SEQ ID NO: 3569), ACGgugagua (SEQ ID NO: 606), CUGguaaugu (SEQ ID NO: 3570), AAGguaucag (SEQ ID NO: 247), CAGgugcccc (SEQ ID NO: 1370), AGUgucagug (SEQ ID NO: 3571), AAGguaggag (SEQ ID NO: 218), GGAguaugug (SEQ ID NO: 3572), UUGguauuuu (SEQ ID NO: 2992), CCUguuguga (SEQ ID NO: 3573), UUUguaagaa (SEQ ID NO: 3033), UAGguaacau (SEQ ID NO: 2475), CAGguaagca (SEQ ID NO: 3574), CAGgucacag (SEQ ID NO: 3575), CAGgugugag (SEQ ID NO: 1432), UAGguuugcg (SEQ ID NO: 3576), CUGguaagaa (SEQ ID NO: 1697), ACGguuguau (SEQ ID NO: 3577), AAGguugggg (SEQ ID NO: 446), AAGgugaauu (SEQ ID NO: 329), GGGguuaguu (SEQ ID NO: 3578), ACGguaaggc (SEQ ID NO: 3579), CAGguuuaag (SEQ ID NO: 1496), CUGguaaguu (SEQ ID NO: 1709), GGGgugagag (SEQ ID NO: 3580), UGGguggguu (SEQ ID NO: 2886), GAGguuuguu (SEQ ID NO: 2111), UGGguaaaug (SEQ ID NO: 2826), CAGgcaggcc (SEQ ID NO: 3581), CACgugcagg (SEQ ID NO: 3582), AAGgugagcc (SEQ ID NO: 340), CAAguaagug (SEQ ID NO: 1028), CAGgucaguc (SEQ ID NO: 1282), GCGguauaau (SEQ ID NO: 3583), UAGguaaagu (SEQ ID NO: 3584), UAGguggauu (SEQ ID NO: 3585), GAGgucugga (SEQ ID NO: 3586), UCGgucaguu (SEQ ID NO: 3587), UGGguaacug (SEQ ID NO: 3588), AAGguuugau (SEQ ID NO: 3589), UGUgcuggug (SEQ ID NO: 3590), UGUguaccuc (SEQ ID NO: 3591), UGGguacagu (SEQ ID NO: 2849), AUCgucagcg (SEQ ID NO: 3592), CAGgucuugg (SEQ ID NO: 3593), GAAguuggua (SEQ ID NO: 3594), GAAguaaaga (SEQ ID NO: 3595), UUGguaagcu (SEQ ID NO: 2959), UAGguaccag (SEQ ID NO: 2507), AGGguaucau (SEQ ID NO: 3596), CAGguaaaaa (SEQ ID NO: 1118), ACGguaauuu (SEQ ID NO: 583), AUUguaaguu (SEQ ID NO: 997), GAGguacagu (SEQ ID NO: 1908), CAGgugaaag (SEQ ID NO: 1315), UGGguuguuu (SEQ ID NO: 3597), GGGguaggug (SEQ ID NO: 2259), CAGgugccca (SEQ ID NO: 1369), AGCgugagau (SEQ ID NO: 3598), CCAgugagug (SEQ ID NO: 1565), AGGguagaug (SEQ ID NO: 3599), UGGguguguc (SEQ ID NO: 2888), AUCgcgugag (SEQ ID NO: 3600), AGGguaagcc (SEQ ID NO: 3601), AGGguagcag (SEQ ID NO: 3602), UUCguuuccg (SEQ ID NO: 3603), AAGguaagcg (SEQ ID NO: 147), UGGguaagcc (SEQ ID NO: 2837), CAGguauggc (SEQ ID NO: 3604), UGUguaagua (SEQ ID NO: 2907), AAGguagaga (SEQ ID NO: 3605), ACGguaauaa (SEQ ID NO: 3606), CUGguacggu (SEQ ID NO: 3607), GAGgucacag (SEQ ID NO: 3608), UAUguaaguu (SEQ ID NO: 2656), CUGguacgcc (SEQ ID NO: 3609), CAAguaagau (SEQ ID NO: 1024), CUAgugagua (SEQ ID NO: 1673), CCGguaaccg (SEQ ID NO: 3610), CUUguaaguc (SEQ ID NO: 3611), GUGgugagaa (SEQ ID NO: 2378), ACCguaugua (SEQ ID NO: 3612), GUAguaagug (SEQ ID NO: 2324), UUGgugggua (SEQ ID NO: 3014), CGGguacuuu (SEQ ID NO: 3613), UGGguaaaua (SEQ ID NO: 2825), AGAgugagua (SEQ ID NO: 704), AAGguagguu (SEQ ID NO: 230), AAGguaugcg (SEQ ID NO: 3614), CCUguaggcu (SEQ ID NO: 3615), ACAguagaaa (SEQ ID NO: 3616), CCGguuagua (SEQ ID NO: 3617), CGGguaggcg (SEQ ID NO: 3618), GCAgugagug (SEQ ID NO: 2162), GAGgugaguc (SEQ ID NO: 3619), CUGguagccu (SEQ ID NO: 3620), CAUguaugua (SEQ ID NO: 1533), GAAguaacuu (SEQ ID NO: 3621), GAAguaagau (SEQ ID NO: 3622), AAGguuagau (SEQ ID NO: 417), AAGguaauca (SEQ ID NO: 161), AAUguaugua (SEQ ID NO: 507), UGAguaagau (SEQ ID NO: 2767), AGAgugagca (SEQ ID NO: 703), GUAguucuau (SEQ ID NO: 3623), GAGguaauca (SEQ ID NO: 1898), UAGguaugga (SEQ ID NO: 2548), UAGgugggac (SEQ ID NO: 2612), GAGguacaug (SEQ ID NO: 3624), UGGguaaggc (SEQ ID NO: 3625), CAGguacgcc (SEQ ID NO: 1182), CCAguuacgc (SEQ ID NO: 3626), ACUgugguga (SEQ ID NO: 3627), GAGguaaguc (SEQ ID NO: 1894), AUUguaggug (SEQ ID NO: 3628), ACCgucagug (SEQ ID NO: 3629), AAUgugaggg (SEQ ID NO: 3630), ACUgugagug (SEQ ID NO: 645), UGGguguggu (SEQ ID NO: 3631), AAGguuggga (SEQ ID NO: 445), AAGguuugga (SEQ ID NO: 464), UCCgugagug (SEQ ID NO: 3632), CGGgugagug (SEQ ID NO: 1642), AGAguaagcu (SEQ ID NO: 664), CAGgcaagcu (SEQ ID NO: 3633), UAGguauauu (SEQ ID NO: 2541), AAAguagcag (SEQ ID NO: 3634), GAGguaaccu (SEQ ID NO: 1880), AAGgugggca (SEQ ID NO: 379), AGGgugagua (SEQ ID NO: 795), UGGguaaggu (SEQ ID NO: 2840), CUUgucagug (SEQ ID NO: 3635), UAGgugcgcu (SEQ ID NO: 3636), GAGgcaaauu (SEQ ID NO: 3637), AGGguaccuc (SEQ ID NO: 3638), CAAgugcgua (SEQ ID NO: 3639), AGAguaagac (SEQ ID NO: 660), GUGguaaaua (SEQ ID NO: 3640), GAUguaagcg (SEQ ID NO: 3641), GAGguaaagc (SEQ ID NO: 1871), UAGgugagua (SEQ ID NO: 2596), CAGguaacau (SEQ ID NO: 1130), CCUguacggc (SEQ ID NO: 3642), UAGguauguc (SEQ ID NO: 2552), UAGguccaua (SEQ ID NO: 3643), GAGgugaaaa (SEQ ID NO: 2003), AAAguacuga (SEQ ID NO: 3644), UUGguaagcg (SEQ ID NO: 3645), CAGgcaagcg (SEQ ID NO: 3646), UUUgcagguu (SEQ ID NO: 3647), CAGguuuaua (SEQ ID NO: 3648), CUGguaaagc (SEQ ID NO: 1686), AUGgugagcu (SEQ ID NO: 958), CAGgugguug (SEQ ID NO: 1419), GUAguaaguu (SEQ ID NO: 3649), CAGguaauac (SEQ ID NO: 3650), CAGgcaaggc (SEQ ID NO: 3651), AAGguaauuu (SEQ ID NO: 171), UUUguccgug (SEQ ID NO: 3652), GAGguagguu (SEQ ID NO: 1939), ACCgugagug (SEQ ID NO: 3653), CAAguaagcu (SEQ ID NO: 3654), ACAgugagua (SEQ ID NO: 560), UUGgugagau (SEQ ID NO: 3000), AAGguagucu (SEQ ID NO: 233), CAGguaaagg (SEQ ID NO: 3655), GGGguaugga (SEQ ID NO: 2264), UUUguaagug (SEQ ID NO: 3040), GUGguaagag (SEQ ID NO: 2344), AGUgugaguu (SEQ ID NO: 838), AAGgcaagcg (SEQ ID NO: 3656), UAAgugagua (SEQ ID NO: 2438), AGGgugagug (SEQ ID NO: 797), AGUguacgug (SEQ ID NO: 3657), AGGgugcgua (SEQ ID NO: 3658), GGCgugagcc (SEQ ID NO: 2238), CGAguuauga (SEQ ID NO: 3659), CAGguaaaga (SEQ ID NO: 1122), UUGgugaaga (SEQ ID NO: 3660), AGGguaaugg (SEQ ID NO: 3661), AAGguccaga (SEQ ID NO: 300), AGUgugaguc (SEQ ID NO: 836), CAGguaauuu (SEQ ID NO: 1159), CAGguaacgc (SEQ ID NO: 3662), CUGguacacu (SEQ ID NO: 3663), CUGguuagug (SEQ ID NO: 1782), CAGguacuug (SEQ ID NO: 3664), CACguaagua (SEQ ID NO: 3665), GUGgugcggc (SEQ ID NO: 3666), GAGgucaguu (SEQ ID NO: 3667), AUGguaugcc (SEQ ID NO: 932), AAGgugugug (SEQ ID NO: 405), CUGguggguc (SEQ ID NO: 1772), CAGgugaggc (SEQ ID NO: 1342), AAGguuaguc (SEQ ID NO: 423), AAGguagcug (SEQ ID NO: 215), GAGgucagga (SEQ ID NO: 1983), GUUguaggua (SEQ ID NO: 3668), UGGguacaag (SEQ ID NO: 3669), AUGguaggug (SEQ ID NO: 924), GAGguaagcc (SEQ ID NO: 1886), AUGgcaagua (SEQ ID NO: 3670), AAGguauauu (SEQ ID NO: 245), GCGgugagag (SEQ ID NO: 3671), AAGgugcuuc (SEQ ID NO: 3672), UAGguacauc (SEQ ID NO: 3673), ACUgugguaa (SEQ ID NO: 3674), GAGguaggcu (SEQ ID NO: 1933), GAGguaugca (SEQ ID NO: 3675), AGGguaguuc (SEQ ID NO: 3676), CAGguauccu (SEQ ID NO: 1241), AGGguaaguc (SEQ ID NO: 741), AGGgucaguu (SEQ ID NO: 779), CAGguuggga (SEQ ID NO: 3677), CAGguggaua (SEQ ID NO: 3678), GGAguagguu (SEQ ID NO: 2220), GAGguaggau (SEQ ID NO: 3679), GGGguuugug (SEQ ID NO: 3680), UAGguaauug (SEQ ID NO: 3681), AAGguaaccc (SEQ ID NO: 136), ACGguaagaa (SEQ ID NO: 3682), GAGguagggg (SEQ ID NO: 1936), CGAguaggug (SEQ ID NO: 1619), UCCguaagug (SEQ ID NO: 2710), UCGguacagg (SEQ ID NO: 3683), CAAguaagcg (SEQ ID NO: 3684), AAGguccgcg (SEQ ID NO: 3685), AAUgugagua (SEQ ID NO: 523), CAGgugaaug (SEQ ID NO: 3686), GUGguaaggc (SEQ ID NO: 2350), AGAgugagug (SEQ ID NO: 706), UCUguauguc (SEQ ID NO: 3687), UGGgugaguc (SEQ ID NO: 2876), UCGguuagua (SEQ ID NO: 3688), GAUguaugca (SEQ ID NO: 3689), GAGguuggug (SEQ ID NO: 3690), GAGguggggc (SEQ ID NO: 2061), UGGgucaguc (SEQ ID NO: 3691), GCAgugagua (SEQ ID NO: 2161), CAGguugcuu (SEQ ID NO: 3692), AGGguagagu (SEQ ID NO: 3693), UAGgucaggu (SEQ ID NO: 2567), CGCguaugua (SEQ ID NO: 3694), GAGguauuaa (SEQ ID NO: 3695), CAGguaaacu (SEQ ID NO: 3696), AAAguaaguu (SEQ ID NO: 24), GGGgucuggc (SEQ ID NO: 3697), GCUguggggu (SEQ ID NO: 3698), UUGguaaguc (SEQ ID NO: 3699), AAGguagaag (SEQ ID NO: 3700), AAUgugaguc (SEQ ID NO: 524), AAGgucagcu (SEQ ID NO: 288), AAGguaagag (SEQ ID NO: 143), AUGgugagga (SEQ ID NO: 3701), AAGguacuuc (SEQ ID NO: 200), AAGguaagaa (SEQ ID NO: 141), CCGguacagc (SEQ ID NO: 3702), GCGgugcgga (SEQ ID NO: 3703), CAGguacaua (SEQ ID NO: 1168), CUGgugagga (SEQ ID NO: 1755), CUGguaggug (SEQ ID NO: 1731), AACguagguu (SEQ ID NO: 3704), AUGgugugug (SEQ ID NO: 975), UUGguacuau (SEQ ID NO: 3705), CAGgucggug (SEQ ID NO: 1300), CAGgcauggg (SEQ ID NO: 3706), AUGguaucuu (SEQ ID NO: 929), AAGguaacua (SEQ ID NO: 137), CAGgugggcg (SEQ ID NO: 3707), CACgugagga (SEQ ID NO: 3708), AAGgugguuc (SEQ ID NO: 392), UGGgcauucu (SEQ ID NO: 3709), AUGguaagcc (SEQ ID NO: 894), AGGgucagug (SEQ ID NO: 778), AGAguacgua (SEQ ID NO: 3710), AAGguaggca (SEQ ID NO: 220), AAGguauuca (SEQ ID NO: 3711), CAGguagauu (SEQ ID NO: 1202), GAGguauuua (SEQ ID NO: 1972), GAGgucuaca (SEQ ID NO: 3712), GUUguagguc (SEQ ID NO: 3713), CAGguacucg (SEQ ID NO: 3714), GUCguauguu (SEQ ID NO: 3715), AAGguacuuu (SEQ ID NO: 202), AGAgugagau (SEQ ID NO: 702), AGUguuggua (SEQ ID NO: 3716), AAUgugagug (SEQ ID NO: 525), AAGguagauu (SEQ ID NO: 3717), AUGguuugua (SEQ ID NO: 988), GAGgccccag (SEQ ID NO: 3718), AUGgucaguu (SEQ ID NO: 3719), UCUguaagga (SEQ ID NO: 3720), CAGgucgggc (SEQ ID NO: 3721), CAGguaagcc (SEQ ID NO: 1142), UAGgucagug (SEQ ID NO: 2569), AGAguaggaa (SEQ ID NO: 683), CUGguacuuc (SEQ ID NO: 3722), CUCguaagca (SEQ ID NO: 1674), CAGguaacua (SEQ ID NO: 1134), CAGguggcug (SEQ ID NO: 1401), UGGguccgua (SEQ ID NO: 3723), GAGguugugc (SEQ ID NO: 3724), CAGgugcgcg (SEQ ID NO: 1377), AAAguauggc (SEQ ID NO: 3725), UGAguacgua (SEQ ID NO: 2779), CUGguacgga (SEQ ID NO: 3726), CAAgugaccu (SEQ ID NO: 3727), AAGgugaugu (SEQ ID NO: 356), AAGgucugca (SEQ ID NO: 3728), AAAguuugua (SEQ ID NO: 75), AAGgugagca (SEQ ID NO: 339), GAUguaagcc (SEQ ID NO: 2119), CAAguaauuu (SEQ ID NO: 1035), CAGgugugug (SEQ ID NO: 1442), UGGgugaggg (SEQ ID NO: 2874), AAGgugaccu (SEQ ID NO: 3729), UAGgugugag (SEQ ID NO: 2621), CAGgcagguc (SEQ ID NO: 3730), UCAguaaguu (SEQ ID NO: 2692), UCAgcaguga (SEQ ID NO: 3731), AAGguaccac (SEQ ID NO: 3732), UAAguaggug (SEQ ID NO: 3733), AAGgucagcc (SEQ ID NO: 286), CAGguaacuc (SEQ ID NO: 1135), AAAguaagag (SEQ ID NO: 13), AAGguagaua (SEQ ID NO: 209), AAGgcaaggg (SEQ ID NO: 99), CAGgugucgg (SEQ ID NO: 3734), CAGguggcua (SEQ ID NO: 3735), GAGguugcca (SEQ ID NO: 3736), CAGgccgugg (SEQ ID NO: 3737), UUGguauaug (SEQ ID NO: 3738), GAGguugagu (SEQ ID NO: 3739), GAGguagguc (SEQ ID NO: 3740), GUGguaagac (SEQ ID NO: 2343), UAGguccuuc (SEQ ID NO: 3741), GAGgcaaguc (SEQ ID NO: 3742), GAGguaacau (SEQ ID NO: 3743), CAGguauauc (SEQ ID NO: 1236), UCGguugguu (SEQ ID NO: 3744), CAGgugaacc (SEQ ID NO: 3745), CAGgucuuuu (SEQ ID NO: 3746), CAGgcauggc (SEQ ID NO: 3747), AAAguacuug (SEQ ID NO: 32), CAGgugauuc (SEQ ID NO: 1356), UUGguagguu (SEQ ID NO: 3748), UAUgugagca (SEQ ID NO: 3749), CAGgugagcg (SEQ ID NO: 1339), AAUguaauaa (SEQ ID NO: 3750), AAAguaaggc (SEQ ID NO: 3751), UAGguuuguc (SEQ ID NO: 2644), UAGgugggag (SEQ ID NO: 2613), GAGguaaguu (SEQ ID NO: 3752), AAGguagccg (SEQ ID NO: 3753), CAGguggugc (SEQ ID NO: 3754), UGAgucaguu (SEQ ID NO: 3755), CUGguaggcc (SEQ ID NO: 3756), CAAguaagga (SEQ ID NO: 3757), CGGguaaggc (SEQ ID NO: 3758), AAGgcgagga (SEQ ID NO: 3759), CAGguaguuc (SEQ ID NO: 1230), CAGguaagga (SEQ ID NO: 1143), CCUgugagug (SEQ ID NO: 1610), AAGguaaaug (SEQ ID NO: 132), CCGguaauua (SEQ ID NO: 3760), CAGguaaguu (SEQ ID NO: 1149), AAGgugguca (SEQ ID NO: 3761), CAGguaccuc (SEQ ID NO: 1177), AUCguaagua (SEQ ID NO: 3762), CCGguacaua (SEQ ID NO: 3763), GCGgugagug (SEQ ID NO: 3764), GAGgugguau (SEQ ID NO: 2067), CUGgugugga (SEQ ID NO: 3765), GAGguaauuc (SEQ ID NO: 3766), CAAguacgua (SEQ ID NO: 3767), UCUguaagug (SEQ ID NO: 2746), AAUguaagug (SEQ ID NO: 491), AGGgucuguu (SEQ ID NO: 783), GAGguacugc (SEQ ID NO: 1918), AGGguaaggc (SEQ ID NO: 738), AAGgcaagag (SEQ ID NO: 95), CAGguggguu (SEQ ID NO: 1416), UAGguuagga (SEQ ID NO: 3768), UGAguaagcu (SEQ ID NO: 2769), AGAguaagag (SEQ ID NO: 661), AUGgcaggug (SEQ ID NO: 3769), UAGgcaagua (SEQ ID NO: 3770), AUGguaggua (SEQ ID NO: 923), GCAgcccgca (SEQ ID NO: 3771), ACGguaaacu (SEQ ID NO: 3772), AGGgugaguu (SEQ ID NO: 798), GUAguagucu (SEQ ID NO: 3773), GUGgcugaaa (SEQ ID NO: 3774), CAGguuaguc (SEQ ID NO: 1456), CUGgugagca (SEQ ID NO: 1753), UCAguaagug (SEQ ID NO: 2691), AAAgugauug (SEQ ID NO: 3775), UAGgucugga (SEQ ID NO: 3776), GAGguguuuc (SEQ ID NO: 3777), AAGguaaauu (SEQ ID NO: 133), CAUguacauc (SEQ ID NO: 3778), AAGguuugaa (SEQ ID NO: 3779), CCAgcaagug (SEQ ID NO: 3780), UAGguaauaa (SEQ ID NO: 3781), GAGgcaagug (SEQ ID NO: 1859), CAAgugauuc (SEQ ID NO: 1071), CAGgucgugg (SEQ ID NO: 3782), GAAguaugcc (SEQ ID NO: 3783), UCGgugcccu (SEQ ID NO: 3784), GAGgucaguc (SEQ ID NO: 3785), CAGgugagac (SEQ ID NO: 1334), UUUgucugua (SEQ ID NO: 3786), CAGguagaua (SEQ ID NO: 3787), UGGguaucag (SEQ ID NO: 3788), UAGgugggcu (SEQ ID NO: 2616), AUGgugagau (SEQ ID NO: 3789), CAGguaacac (SEQ ID NO: 3790), CCGguauccu (SEQ ID NO: 3791), UAGguaagcu (SEQ ID NO: 2487), UCAguacauc (SEQ ID NO: 3792), UAGguuugcc (SEQ ID NO: 2642), AUGguaagaa (SEQ ID NO: 889), UUGguaagac (SEQ ID NO: 3793), CCGguuaguc (SEQ ID NO: 3794), GAGguaagaa (SEQ ID NO: 1882), UGGguaaguu (SEQ ID NO: 2844), CCGgugagaa (SEQ ID NO: 1585), CCUgugaggg (SEQ ID NO: 1608), ACGguaggag (SEQ ID NO: 590), ACAguauguc (SEQ ID NO: 3795), CAGguauuaa (SEQ ID NO: 3796), CAGguggauc (SEQ ID NO: 3797), AGAgugcgua (SEQ ID NO: 3798), AAGgugaccg (SEQ ID NO: 3799), AGAguaggug (SEQ ID NO: 687), ACUguaugua (SEQ ID NO: 3800), UAGgucaauu (SEQ ID NO: 3801), AGUguguaag (SEQ ID NO: 3802), CGGguaccuu (SEQ ID NO: 3803), CUAgugaguu (SEQ ID NO: 3804), CUAguaagug (SEQ ID NO: 1666), CAGguacaac (SEQ ID NO: 3805), UAGgugugug (SEQ ID NO: 2627), CAUguacggc (SEQ ID NO: 3806), AUGgugugag (SEQ ID NO: 3807), AGGguggaag (SEQ ID NO: 3808), CAGgugcgag (SEQ ID NO: 3809), UAGgugcucc (SEQ ID NO: 3810), AAGguggugg (SEQ ID NO: 390), AAGgucuguu (SEQ ID NO: 317), CAGgugggcc (SEQ ID NO: 1407), AAGgucaguc (SEQ ID NO: 294), CAGguuuuua (SEQ ID NO: 3811), AACgugaggu (SEQ ID NO: 3812), CGGguaagag (SEQ ID NO: 3813), UUUgucggua (SEQ ID NO: 3814), UAGguuaagu (SEQ ID NO: 3815), GUGguaagaa (SEQ ID NO: 2342), CAGguauugg (SEQ ID NO: 1266), GCUguaaguu (SEQ ID NO: 2196), CUAguaagua (SEQ ID NO: 1664), UCGguaaaua (SEQ ID NO: 3816), CAGguaacuu (SEQ ID NO: 1137), CCUgugagua (SEQ ID NO: 3817), CAGguuauau (SEQ ID NO: 3818), CUGgugaaca (SEQ ID NO: 3819), AAGguauaaa (SEQ ID NO: 238), GAGguaagca (SEQ ID NO: 1885), AAGgugaagc (SEQ ID NO: 324), CAGgugaguu (SEQ ID NO: 1348), UUUgugagua (SEQ ID NO: 3820), CUUguacgcc (SEQ ID NO: 3821), AGAguaagug (SEQ ID NO: 670), UGGguaggug (SEQ ID NO: 2853), UGAgcccuge (SEQ ID NO: 3822), UGUguaugua (SEQ ID NO: 3823), AAGguagagg (SEQ ID NO: 3824), GAGguggggg (SEQ ID NO: 2062), UAGguaauuc (SEQ ID NO: 2502), AAGgcauggu (SEQ ID NO: 3825), AGAguaagca (SEQ ID NO: 663), AAGguaggaa (SEQ ID NO: 217), CAAguaagua (SEQ ID NO: 1026), ACUguaauug (SEQ ID NO: 3826), CAGgucugug (SEQ ID NO: 1311), UCGguaccga (SEQ ID NO: 3827), CUGgugagag (SEQ ID NO: 3828), AAGguuugcu (SEQ ID NO: 463), AUGguaccac (SEQ ID NO: 3829), UAAguuaguu (SEQ ID NO: 3830), CAGguaggac (SEQ ID NO: 1213), AGAgugaggc (SEQ ID NO: 3831), CGAgucagua (SEQ ID NO: 3832), CAGgucugag (SEQ ID NO: 1304), GAGguggugg (SEQ ID NO: 3833), ACGguauugg (SEQ ID NO: 3834), GCUgcgagua (SEQ ID NO: 3835), CUGguaagug (SEQ ID NO: 1708), GUGgugagau (SEQ ID NO: 2379), GGGguuugau (SEQ ID NO: 3836), UCUgugagug (SEQ ID NO: 2762), CUUgucagua (SEQ ID NO: 1801), GAGguaaaac (SEQ ID NO: 1866), UCUguaagau (SEQ ID NO: 2741), CCAguaaguu (SEQ ID NO: 1558), CAGguaaagu (SEQ ID NO: 1124), GCGgugagca (SEQ ID NO: 2179), UAAguaagag (SEQ ID NO: 2416), CUGgcaggug (SEQ ID NO: 3837), GAGguaaggg (SEQ ID NO: 1891), UGAguaaguu (SEQ ID NO: 2775), GAGgugagac (SEQ ID NO: 2015), GCUgucuguu (SEQ ID NO: 3838), AAGguaacaa (SEQ ID NO: 134), GAGguaacgg (SEQ ID NO: 3839), CUGguauucu (SEQ ID NO: 3840), CAAguaacug (SEQ ID NO: 1021), AAGguggggu (SEQ ID NO: 383), UAGguauggc (SEQ ID NO: 2549), CAGguauuuu (SEQ ID NO: 1271), GUGguaaacu (SEQ ID NO: 3841), GAGgucugag (SEQ ID NO: 1998), CUGguaaggu (SEQ ID NO: 1706), CAAguaaguu (SEQ ID NO: 1029), AAGguagacc (SEQ ID NO: 206), GAGgcgagcg (SEQ ID NO: 3842), CUGguaaaua (SEQ ID NO: 1687), UGUguaagcg (SEQ ID NO: 3843), CAGguuaggg (SEQ ID NO: 1453), GGGgugagga (SEQ ID NO: 2280), ACAguaugug (SEQ ID NO: 3844), CCGgugggga (SEQ ID NO: 3845), GAGgucagug (SEQ ID NO: 3846), AGGguaaggu (SEQ ID NO: 3847), ACAguaagua (SEQ ID NO: 546), GGUguaaggu (SEQ ID NO: 3848), GAGguaauaa (SEQ ID NO: 1895), CAGguauucc (SEQ ID NO: 3849), CUGguauaaa (SEQ ID NO: 3850), CCGgucugug (SEQ ID NO: 3851), CAGguaacug (SEQ ID NO: 1136), GCAguaagua (SEQ ID NO: 2147), AAGguagggg (SEQ ID NO: 225), CAAguccacc (SEQ ID NO: 3852), CAAguuggug (SEQ ID NO: 3853), CAGgugcggu (SEQ ID NO: 1379), CAGguaaaau (SEQ ID NO: 3854), ACGguaagga (SEQ ID NO: 3855), UGGguaauaa (SEQ ID NO: 3856), UAGguaagug (SEQ ID NO: 2493), CCGguagguu (SEQ ID NO: 3857), AGAguaugga (SEQ ID NO: 3858), CUCgugaguc (SEQ ID NO: 3859), AAAgccggug (SEQ ID NO: 3860), UUGguaauuu (SEQ ID NO: 2970), GAGguaaaag (SEQ ID NO: 1867), CCUgugugag (SEQ ID NO: 3861), AAAguaagga (SEQ ID NO: 18), UGAgugagug (SEQ ID NO: 2800), AAGguacaug (SEQ ID NO: 180), CCGguaaaug (SEQ ID NO: 3862), CAGgugaagc (SEQ ID NO: 3863), CAGguacccg (SEQ ID NO: 1173), GAGguaaggc (SEQ ID NO: 1890), UUUguauguu (SEQ ID NO: 3049), CAGgugcucc (SEQ ID NO: 1386), UCGguagguc (SEQ ID NO: 3864), CGGgugaggc (SEQ ID NO: 3865), AAGguaauua (SEQ ID NO: 168), ACUgugaguc (SEQ ID NO: 644), AAGgucagca (SEQ ID NO: 285), GUGgugagug (SEQ ID NO: 2384), CAUguccacc (SEQ ID NO: 3866), AAGgugaccc (SEQ ID NO: 3867), CGGguuagua (SEQ ID NO: 3868), GCGguaguaa (SEQ ID NO: 3869), GCUguaggua (SEQ ID NO: 3870), CCUguugagu (SEQ ID NO: 3871), UAGgucuggc (SEQ ID NO: 2577), GAUgugagcc (SEQ ID NO: 2131), CUUgugagua (SEQ ID NO: 1802), CUGguguguu (SEQ ID NO: 1780), GAGgcaugug (SEQ ID NO: 1863), CAGgcaagag (SEQ ID NO: 1101), UUGguaagaa (SEQ ID NO: 2957), GAGguguggg (SEQ ID NO: 2075), GAGguauuuu (SEQ ID NO: 1975), CAGguaguaa (SEQ ID NO: 1224), AGGguaagac (SEQ ID NO: 3872), UUUguaggca (SEQ ID NO: 3873), AGGgugagau (SEQ ID NO: 3874), GAGguuugua (SEQ ID NO: 2110), AAGgugagug (SEQ ID NO: 349), GAGgugggag (SEQ ID NO: 2055), AAGgugagaa (SEQ ID NO: 335), CUGguaagag (SEQ ID NO: 1698), AUAguaaaga (SEQ ID NO: 3875), GAUgugaguc (SEQ ID NO: 2134), AAGgugcagg (SEQ ID NO: 3876), CAGgucuguc (SEQ ID NO: 1310), GAGgugauuu (SEQ ID NO: 3877), CAGguuggcu (SEQ ID NO: 3878), CGGguauggg (SEQ ID NO: 3879), AUGguccauc (SEQ ID NO: 3880), CCGguuggug (SEQ ID NO: 3881), GGAguaaguc (SEQ ID NO: 3882), AAUguaagga (SEQ ID NO: 488), CAGguuuguu (SEQ ID NO: 1510), UAGgugugua (SEQ ID NO: 2626), UAUgucuuug (SEQ ID NO: 3883), ACGguacuuc (SEQ ID NO: 3884), AAGgcacgcg (SEQ ID NO: 3885), CUGguaaacc (SEQ ID NO: 1684), CUUgugggua (SEQ ID NO: 3886), UGAguaaguc (SEQ ID NO: 2773), CUGgugggug (SEQ ID NO: 1773), GAGguggaga (SEQ ID NO: 3887), GUGguggcug (SEQ ID NO: 3888), GUGguaagug (SEQ ID NO: 2353), AACgugagua (SEQ ID NO: 3889), GAAgcuguaa (SEQ ID NO: 3890), CGGguaucuu (SEQ ID NO: 3891), CAGgugucag (SEQ ID NO: 1424), AAUguacgca (SEQ ID NO: 3892), CCGgugggua (SEQ ID NO: 3893), UGGgugaggu (SEQ ID NO: 3894), AAGguauguu (SEQ ID NO: 266), CAGguauguu (SEQ ID NO: 1261), CAGguuugcu (SEQ ID NO: 1505), UUGguaaguu (SEQ ID NO: 2964), CAGguaguug (SEQ ID NO: 1231), CCUgugaaua (SEQ ID NO: 3895), GCUgugugug (SEQ ID NO: 3896), CAAguaauuc (SEQ ID NO: 1033), AGGguaaugu (SEQ ID NO: 3897), GCUgugaguc (SEQ ID NO: 2205), ACCguaaguu (SEQ ID NO: 3898), CGUguaagua (SEQ ID NO: 3899), GGGguaaguc (SEQ ID NO: 3900), AAUguaugau (SEQ ID NO: 3901), AAUgugauua (SEQ ID NO: 3902), UCAguaagaa (SEQ ID NO: 2682), CAGguccguc (SEQ ID NO: 3903), GAAguauuga (SEQ ID NO: 3904), UUGguaagga (SEQ ID NO: 2960), CAGgucgguu (SEQ ID NO: 3905), UAGguuagug (SEQ ID NO: 2635), ACGguaaaac (SEQ ID NO: 577), AAGguagguc (SEQ ID NO: 228), UACgugagua (SEQ ID NO: 3906), UUGguaagca (SEQ ID NO: 3907), GCGgugaguc (SEQ ID NO: 3908), GAAguaaggg (SEQ ID NO: 3909), CGCgugaguu (SEQ ID NO: 3910), CAGguacccc (SEQ ID NO: 3911), UCUguaagac (SEQ ID NO: 3912), GAGgugggca (SEQ ID NO: 2057), AAUguaagac (SEQ ID NO: 3913), CAGgcaaggg (SEQ ID NO: 3914), CAAguaacua (SEQ ID NO: 1020), AAAguuuguc (SEQ ID NO: 3915), CAGguacugu (SEQ ID NO: 1193), AAGgucccuc (SEQ ID NO: 303), UCGguaaguc (SEQ ID NO: 3916), UGGgugagug (SEQ ID NO: 2877), CUUgugagau (SEQ ID NO: 3917), AGAgugagcu (SEQ ID NO: 3918), UAAgugggga (SEQ ID NO: 3919), UAGguaggga (SEQ ID NO: 2522), CAGguuagcc (SEQ ID NO: 1452), AGGguaauca (SEQ ID NO: 3920), AAGguucagc (SEQ ID NO: 3921), UGGgugggug (SEQ ID NO: 2885), CAGguuguga (SEQ ID NO: 1494), AAGguaagug (SEQ ID NO: 155), CAUgugcgua (SEQ ID NO: 1543), CCGguauauu (SEQ ID NO: 3922), ACCguaugug (SEQ ID NO: 3923), CAGguauagu (SEQ ID NO: 3924), CAGguauuac (SEQ ID NO: 3925), CAGgugcagg (SEQ ID NO: 1364), GUGgugagcu (SEQ ID NO: 2381), AAGguaacau (SEQ ID NO: 135), CUGgugaugg (SEQ ID NO: 3926), AUGguaaaug (SEQ ID NO: 882), CCGgugagca (SEQ ID NO: 3927), AAGguaaacc (SEQ ID NO: 124), AAGguacugg (SEQ ID NO: 3928), GCGgucagga (SEQ ID NO: 3929), CUGgucaggg (SEQ ID NO: 3930), AAAguacguu (SEQ ID NO: 3931), AGAguagguu (SEQ ID NO: 688), AGGguaagcu (SEQ ID NO: 3932), AUUgugagua (SEQ ID NO: 1009), CCGgccacca (SEQ ID NO: 3933), GAGguaacuu (SEQ ID NO: 1881), GAGguaugaa (SEQ ID NO: 1956), CAGgucagac (SEQ ID NO: 1276), UAGgcgugug (SEQ ID NO: 2462), AGGguaaguu (SEQ ID NO: 743), CAGgcaugag (SEQ ID NO: 1111), CAGguaacgu (SEQ ID NO: 1133), CAGgcgagca (SEQ ID NO: 3934), UAGguauggu (SEQ ID NO: 2550), AGAguaggau (SEQ ID NO: 3935), CUGguuucaa (SEQ ID NO: 3936), GAGguaaacu (SEQ ID NO: 3937), CAGgcaugca (SEQ ID NO: 1112), UUGguaaucu (SEQ ID NO: 3938), AGGgcagaau (SEQ ID NO: 3939), AUGguaaaac (SEQ ID NO: 877), GCUgcaggug (SEQ ID NO: 3940), GAAgcacgug (SEQ ID NO: 3941), CAUguaaaca (SEQ ID NO: 3942), UGGguaagau (SEQ ID NO: 2835), AGGguagcua (SEQ ID NO: 3943), AGGguggggu (SEQ ID NO: 800), CCUguaaguu (SEQ ID NO: 1600), UGAgugaguu (SEQ ID NO: 2801), GGAguaugua (SEQ ID NO: 3944), CAGgugaccu (SEQ ID NO: 1328), AAAguacgga (SEQ ID NO: 3945), GAGguacaga (SEQ ID NO: 1906), GAUguaggua (SEQ ID NO: 2125), GGGguaauug (SEQ ID NO: 3946), UAGguggguu (SEQ ID NO: 2617), GUGguacgua (SEQ ID NO: 3947), AAGguacagc (SEQ ID NO: 3948), GAGgugaaga (SEQ ID NO: 3949), GGGguaagca (SEQ ID NO: 2246), UGAguagguc (SEQ ID NO: 3950), GGGguaaguu (SEQ ID NO: 2253), AUUgugaguu (SEQ ID NO: 1011), UCAguaagac (SEQ ID NO: 3951), AGUgugagcu (SEQ ID NO: 834), AAGgcaaaac (SEQ ID NO: 3952), CUGgugaguc (SEQ ID NO: 1760), AAGgucucug (SEQ ID NO: 310), GAGgcugugc (SEQ ID NO: 3953), AGAgugagac (SEQ ID NO: 700), GAGgugaugu (SEQ ID NO: 2033), AGAguauggu (SEQ ID NO: 3954), UGGguggguc (SEQ ID NO: 2884), GCUgcugagc (SEQ ID NO: 3955), CAGguagcug (SEQ ID NO: 1210), UAGgucagaa (SEQ ID NO: 3956), CCGguaggug (SEQ ID NO: 3957), GCAguaugau (SEQ ID NO: 3958), CAGguuucag (SEQ ID NO: 3959), GAGguuugcc (SEQ ID NO: 3960), GGGguggggg (SEQ ID NO: 3961), AAGguacaua (SEQ ID NO: 179), UGGguguguu (SEQ ID NO: 2890), AGAguaaggc (SEQ ID NO: 666), GCGguuagug (SEQ ID NO: 3962), AAGgugacuu (SEQ ID NO: 334), AUGguaagau (SEQ ID NO: 892), AUGguaguug (SEQ ID NO: 3963), CAUguaagac (SEQ ID NO: 3964), CUGguaugua (SEQ ID NO: 1736), UUCguaagga (SEQ ID NO: 3965), GAAguaugac (SEQ ID NO: 3966), CGGguaauuc (SEQ ID NO: 1627), UGGguaacuu (SEQ ID NO: 2831), CAGgugccua (SEQ ID NO: 1372), CAUguagggc (SEQ ID NO: 3967), ACCgucagga (SEQ ID NO: 3968), CGUguucgau (SEQ ID NO: 3969), GAGgcaggac (SEQ ID NO: 3970), UAGguaauau (SEQ ID NO: 2496), UCGguauacu (SEQ ID NO: 3971), UAGguugugc (SEQ ID NO: 3972), CCGgugaguc (SEQ ID NO: 3973), CAGgugccaa (SEQ ID NO: 1368), CAGgugaugc (SEQ ID NO: 1352), AAGgugagga (SEQ ID NO: 343), GUGgugaggg (SEQ ID NO: 3974), UGGgucagua (SEQ ID NO: 3975), GAGgucaggg (SEQ ID NO: 1985), UAGguacgua (SEQ ID NO: 2511), GAGgcaagag (SEQ ID NO: 1857), CCUguuggua (SEQ ID NO: 3976), GAGguaucca (SEQ ID NO: 3977), UAAguaagcu (SEQ ID NO: 2419), AAGgucaguu (SEQ ID NO: 296), AAAguuaaag (SEQ ID NO: 3978), GAGgugcuau (SEQ ID NO: 3979), ACGguaaguu (SEQ ID NO: 581), CUGgugaggg (SEQ ID NO: 1757), GAGguuaugu (SEQ ID NO: 2091), CUUgugugca (SEQ ID NO: 3980), UGAgcugggg (SEQ ID NO: 3981), AAGguauagu (SEQ ID NO: 3982), UAGguaaaac (SEQ ID NO: 2464), GGGgugaggu (SEQ ID NO: 3983), GAGgcaagca (SEQ ID NO: 3984), GGAguaacgu (SEQ ID NO: 3985), AGAguaagua (SEQ ID NO: 3986), AAAguaagua (SEQ ID NO: 21), GAGgcaacca (SEQ ID NO: 3987), UGUguaaguu (SEQ ID NO: 2909), UAGgugaggc (SEQ ID NO: 2594), ACAguaagaa (SEQ ID NO: 544), UGAguaagug (SEQ ID NO: 2774), CAAgucagua (SEQ ID NO: 1057), AGGguaaaug (SEQ ID NO: 3988), AAGguaugca (SEQ ID NO: 257), GCUgugcgug (SEQ ID NO: 3989), GAGguucgcc (SEQ ID NO: 3990), AAGgcuugca (SEQ ID NO: 3991), CAGgcaagug (SEQ ID NO: 1104), AUAguaaguc (SEQ ID NO: 3992), UUGguaggua (SEQ ID NO: 2978), GCAgcaggua (SEQ ID NO: 3993), AAGguauauc (SEQ ID NO: 243), AGCguaagcc (SEQ ID NO: 3994), CUGguucgaa (SEQ ID NO: 3995), ACGgugggug (SEQ ID NO: 612), CUGgucauug (SEQ ID NO: 3996), CAGgucagga (SEQ ID NO: 1280), CAAgugagac (SEQ ID NO: 1062), GAGguacugg (SEQ ID NO: 1919), GAGguguagu (SEQ ID NO: 3997), GAGguguccu (SEQ ID NO: 3998), CAGgugcgua (SEQ ID NO: 1380), AGUgcccuga (SEQ ID NO: 3999), AUGgugaguc (SEQ ID NO: 962), UGUgugugua (SEQ ID NO: 4000), CAGguaugcu (SEQ ID NO: 1254), CUGguacagu (SEQ ID NO: 4001), UUGguacgua (SEQ ID NO: 4002), UCUguacgua (SEQ ID NO: 4003), UAAguaauuc (SEQ ID NO: 4004), CACguaugug (SEQ ID NO: 4005), CAGgcaagua (SEQ ID NO: 1103), UCGgugagug (SEQ ID NO: 4006), GGUgugaguc (SEQ ID NO: 2315), UCUguaagcu (SEQ ID NO: 2743), AAGguucaga (SEQ ID NO: 4007), AGGguacuuc (SEQ ID NO: 4008), GCGgcagguu (SEQ ID NO: 4009), GAGgcccgug (SEQ ID NO: 4010), CAGguauaaa (SEQ ID NO: 4011), AUGgucaagu (SEQ ID NO: 4012), AAGgugagua (SEQ ID NO: 347), GUGguuuguu (SEQ ID NO: 4013), AGAgugagga (SEQ ID NO: 4014), GAGguaugac (SEQ ID NO: 1957), UAGgcgugag (SEQ ID NO: 4015), AAGguacucc (SEQ ID NO: 4016), UGAgugagga (SEQ ID NO: 2798), GAGguaugau (SEQ ID NO: 4017), GGGgucggua (SEQ ID NO: 4018), ACGguaugca (SEQ ID NO: 4019), CAGguaccac (SEQ ID NO: 1171), UAAguaccug (SEQ ID NO: 4020), AGGgugggcu (SEQ ID NO: 4021), CUGgucuguu (SEQ ID NO: 4022), UAGgucagag (SEQ ID NO: 4023), AAGguguguu (SEQ ID NO: 406), CUGgucagug (SEQ ID NO: 4024), AAGgugggac (SEQ ID NO: 4025), GUGguaguag (SEQ ID NO: 4026), CUAguuuagg (SEQ ID NO: 4027), CCCgccccau (SEQ ID NO: 4028), GCUguacugc (SEQ ID NO: 4029), GAGguaauau (SEQ ID NO: 1897), UAGguuggug (SEQ ID NO: 4030), AAGguccaac (SEQ ID NO: 4031), UAGgugagga (SEQ ID NO: 2593), GUGguaaguu (SEQ ID NO: 2354), AGUgugagag (SEQ ID NO: 831), AAUguacaug (SEQ ID NO: 497), UUGgcaggug (SEQ ID NO: 4032), UAGguuauug (SEQ ID NO: 4033), CAGguacuga (SEQ ID NO: 1191), GCGguggguc (SEQ ID NO: 4034), UGUguaagau (SEQ ID NO: 4035), GAGgugagua (SEQ ID NO: 2025), GCAgccccgg (SEQ ID NO: 4036), CAGgugcuaa (SEQ ID NO: 4037), AGUguaagag (SEQ ID NO: 815), CAGguacauc (SEQ ID NO: 4038), CAGgugggac (SEQ ID NO: 1403), AGGguaaaua (SEQ ID NO: 727), UAAguaauua (SEQ ID NO: 4039), CAGguaaccg (SEQ ID NO: 1132), AAGguuugca (SEQ ID NO: 461), UAGgugguuu (SEQ ID NO: 4040), CAGgugaccg (SEQ ID NO: 1327), UGUguaagcu (SEQ ID NO: 4041), GGAgugaguc (SEQ ID NO: 2227), AGGguaggag (SEQ ID NO: 752), AGGgugggug (SEQ ID NO: 802), AAGgucugag (SEQ ID NO: 313), GAUguaauau (SEQ ID NO: 4042), GGGguaauua (SEQ ID NO: 4043), UAGguaggua (SEQ ID NO: 2524), GAGgcaagua (SEQ ID NO: 1858), GAGguaagga (SEQ ID NO: 1889), UAGguacuac (SEQ ID NO: 4044), UCGgugggug (SEQ ID NO: 4045), AAGgugugga (SEQ ID NO: 401), CAGgucugcc (SEQ ID NO: 1305), UAAgugagcc (SEQ ID NO: 4046), GAAguaaguu (SEQ ID NO: 1820), GAAguaagcc (SEQ ID NO: 1815), UAGgugcgac (SEQ ID NO: 4047), GAGguauggc (SEQ ID NO: 4048), GCAguaagaa (SEQ ID NO: 2145), CAGgugugga (SEQ ID NO: 1438), UUGguaacgu (SEQ ID NO: 4049), GCUguaaaaa (SEQ ID NO: 4050), UUGguuagua (SEQ ID NO: 4051), AUAguaaggg (SEQ ID NO: 4052), UUGguacuag (SEQ ID NO: 4053), CGGgcagccg (SEQ ID NO: 4054), CAGgugcugg (SEQ ID NO: 1389), UAUgugaguu (SEQ ID NO: 2673), CAGgucuggg (SEQ ID NO: 4055), UAAguaagaa (SEQ ID NO: 2415), AAGguuauua (SEQ ID NO: 4056), AGAguaaagc (SEQ ID NO: 4057), AGAgugugag (SEQ ID NO: 4058), UAGgugcgag (SEQ ID NO: 4059), CAAguaaacg (SEQ ID NO: 4060), AAGguacgua (SEQ ID NO: 4061), CUGgugagua (SEQ ID NO: 1759), CCAguaugua (SEQ ID NO: 4062), UUGgugagug (SEQ ID NO: 3006), UGAguaagua (SEQ ID NO: 2772), GAGguuagca (SEQ ID NO: 4063), GUGguaagcc (SEQ ID NO: 4064), CUGguauggc (SEQ ID NO: 1734), AAAguaacac (SEQ ID NO: 8), CAGguacuaa (SEQ ID NO: 1186), UCUguaaguu (SEQ ID NO: 2747), GAGgugaggg (SEQ ID NO: 2024), ACUgugggua (SEQ ID NO: 647), GAUguuugug (SEQ ID NO: 4065), CAGgugucaa (SEQ ID NO: 4066), CAGgucacca (SEQ ID NO: 4067), CCGgugagua (SEQ ID NO: 4068), UUGguaaaua (SEQ ID NO: 4069), CAGguggggg (SEQ ID NO: 1411), ACUgcaggug (SEQ ID NO: 4070), UAGguauguu (SEQ ID NO: 2554), GGAgcaagug (SEQ ID NO: 4071), UCGgugccuc (SEQ ID NO: 4072), CAAguaacuu (SEQ ID NO: 4073), GAGguaacca (SEQ ID NO: 1879), CAGguaauau (SEQ ID NO: 1151), GGAguaagaa (SEQ ID NO: 4074), GAGguaccuu (SEQ ID NO: 1914), AGGguaagga (SEQ ID NO: 737), CCUgugaguc (SEQ ID NO: 1609), GAGguaaugg (SEQ ID NO: 1900), AUGguguguc (SEQ ID NO: 4075), GGGgugagua (SEQ ID NO: 4076), AGGgucaggu (SEQ ID NO: 4077), UGGguaaggg (SEQ ID NO: 2839), AGGguagguu (SEQ ID NO: 759), AUAgugaguu (SEQ ID NO: 4078), CCCguaggcu (SEQ ID NO: 4079), ACAguaugua (SEQ ID NO: 553), GACgugugua (SEQ ID NO: 4080), GCGgugagga (SEQ ID NO: 4081), CAGgugaccc (SEQ ID NO: 1326), UAAguuuagu (SEQ ID NO: 4082), ACAguugagu (SEQ ID NO: 570), CGGgugaggg (SEQ ID NO: 1639), CAGguggauu (SEQ ID NO: 1398), CGGguagagg (SEQ ID NO: 4083), UAGgugcgug (SEQ ID NO: 2608), GGGguaagaa (SEQ ID NO: 2243), GAGguggggu (SEQ ID NO: 4084), CACguggguu (SEQ ID NO: 4085), ACGguaauug (SEQ ID NO: 4086), AGAgugaguc (SEQ ID NO: 705), UUGgcuccaa (SEQ ID NO: 4087), AAGgugaugc (SEQ ID NO: 355), AAGguugguc (SEQ ID NO: 448), AGCguaaguu (SEQ ID NO: 4088), AUUguaugua (SEQ ID NO: 1006), UCAguuaagu (SEQ ID NO: 4089), CAAguacgug (SEQ ID NO: 4090), CAGgugcgug (SEQ ID NO: 1382), CAGguaggua (SEQ ID NO: 1220), AUGguggggu (SEQ ID NO: 4091), AUGgugaguu (SEQ ID NO: 964), CAGguaauca (SEQ ID NO: 4092), AAGguagggu (SEQ ID NO: 226), CAGgccaagg (SEQ ID NO: 4093), GUGgugagag (SEQ ID NO: 4094), AAGguuggug (SEQ ID NO: 449), CAGguacucu (SEQ ID NO: 1190), UAGgcaugug (SEQ ID NO: 4095), UUGguaccuu (SEQ ID NO: 4096), CUGgugugcc (SEQ ID NO: 4097), ACAguugcca (SEQ ID NO: 4098), UUGguaauau (SEQ ID NO: 4099), GAGgugcaug (SEQ ID NO: 4100), UUGguuugua (SEQ ID NO: 3028), UUGguaagug (SEQ ID NO: 2963), UGUgugugug (SEQ ID NO: 4101), GUGguuugua (SEQ ID NO: 2398), GCGguacaca (SEQ ID NO: 4102), AGAguaugcu (SEQ ID NO: 4103), UUUguaagua (SEQ ID NO: 3038), UCUgugcggg (SEQ ID NO: 4104), AAGgucagug (SEQ ID NO: 295), GAGguaggaa (SEQ ID NO: 1930), GCGguuagca (SEQ ID NO: 4105), AGGgugaggg (SEQ ID NO: 793), GAAgugagua (SEQ ID NO: 4106), CAGgugacag (SEQ ID NO: 4107), AAGgugauua (SEQ ID NO: 357), GAGgccagcc (SEQ ID NO: 4108), GAGgucuccu (SEQ ID NO: 4109), UAGguauuac (SEQ ID NO: 2556), CAUguaagag (SEQ ID NO: 1519), CUGguagggc (SEQ ID NO: 4110), GAAguaagua (SEQ ID NO: 1818), CGGguaagug (SEQ ID NO: 4111), CAGguaaucu (SEQ ID NO: 4112), GUGguaggua (SEQ ID NO: 4113), CAGgugggua (SEQ ID NO: 1413), AAGgccagug (SEQ ID NO: 4114), AAAgugaauc (SEQ ID NO: 4115), ACGguuacgu (SEQ ID NO: 4116), AUGguaggaa (SEQ ID NO: 917), CGGgugagac (SEQ ID NO: 4117), GAGguuggaa (SEQ ID NO: 2099), UGGgugagcc (SEQ ID NO: 2871), CCAgugagua (SEQ ID NO: 1564), CUAguacgag (SEQ ID NO: 4118), CAGguaugac (SEQ ID NO: 1248), GCUgugaggu (SEQ ID NO: 4119), CUGguaugaa (SEQ ID NO: 4120), GGUguacgac (SEQ ID NO: 4121), CUUgugagug (SEQ ID NO: 4122), GUGgugagca (SEQ ID NO: 2380), CUGguaacuu (SEQ ID NO: 1696), CAGguacuau (SEQ ID NO: 1188), AGGguaaggg (SEQ ID NO: 739), UUGguuaguu (SEQ ID NO: 3025), GGUguaagca (SEQ ID NO: 2302), UCGgugagga (SEQ ID NO: 4123), UGGguaaaca (SEQ ID NO: 4124), UCGguacgug (SEQ ID NO: 4125), UAGguagcag (SEQ ID NO: 4126), CUGguaaggc (SEQ ID NO: 1704), GUGguaagga (SEQ ID NO: 2349), UAAguaagca (SEQ ID NO: 2418), GAGguuccaa (SEQ ID NO: 4127), CUGguaugga (SEQ ID NO: 4128), GGGgugggua (SEQ ID NO: 2288), CAGguuuccc (SEQ ID NO: 4129), CAGgucucug (SEQ ID NO: 4130), GAGgugagga (SEQ ID NO: 2022), CUUguggguu (SEQ ID NO: 1805), AUGgugagac (SEQ ID NO: 953), CAGgugaagg (SEQ ID NO: 1319), GCGguagggg (SEQ ID NO: 4131), GUUguuuccc (SEQ ID NO: 4132), AAAgcaucca (SEQ ID NO: 4133), GUGguagguu (SEQ ID NO: 2367), AAGgugugaa (SEQ ID NO: 398), CAGguacagu (SEQ ID NO: 1167), AAGguaccaa (SEQ ID NO: 182), UUGguaauug (SEQ ID NO: 2969), AAGgugcuca (SEQ ID NO: 4134), AAGguucaac (SEQ ID NO: 4135), CAGguuuaca (SEQ ID NO: 4136), GCUguaagug (SEQ ID NO: 2195), AGGguauguc (SEQ ID NO: 769), GAGgucgggg (SEQ ID NO: 1996), AAGgugccug (SEQ ID NO: 363), AAGguaaaaa (SEQ ID NO: 119), GUGgugaguu (SEQ ID NO: 2385), UAGguaagaa (SEQ ID NO: 4137), AGGguauccu (SEQ ID NO: 4138), GUGguaauau (SEQ ID NO: 4139), UCUguaagua (SEQ ID NO: 2744), UGGguaugga (SEQ ID NO: 4140), AUGguaugga (SEQ ID NO: 935), GACgugagcc (SEQ ID NO: 1854), CUGguuuggc (SEQ ID NO: 4141), AUGguauauc (SEQ ID NO: 4142), AAAguaaacu (SEQ ID NO: 4143), AGCgugagug (SEQ ID NO: 721), CUGguauaga (SEQ ID NO: 4144), CAGgugggga (SEQ ID NO: 1409), AGAguauguu (SEQ ID NO: 696), UAGguacuug (SEQ ID NO: 4145), GCAguaggug (SEQ ID NO: 4146), AGUguauguc (SEQ ID NO: 4147), AAGguuaagc (SEQ ID NO: 413), CUGguggccu (SEQ ID NO: 4148), GAAgugaguc (SEQ ID NO: 1839), UUGguguaag (SEQ ID NO: 4149), CAGguaagaa (SEQ ID NO: 1138), CGGgucucgg (SEQ ID NO: 4150), GAGgugcaca (SEQ ID NO: 2035), CUCguuaguu (SEQ ID NO: 4151), AAGgugauca (SEQ ID NO: 352), UAUguaagaa (SEQ ID NO: 2649), GAGgugcuug (SEQ ID NO: 2047), CAGgugguca (SEQ ID NO: 4152), ACGguaaguc (SEQ ID NO: 4153), ACAguaaugu (SEQ ID NO: 4154), CCUguaaggu (SEQ ID NO: 4155), GAGguuaagu (SEQ ID NO: 4156), UCGguaugug (SEQ ID NO: 2725), UGGguauguu (SEQ ID NO: 2863), AAGguauuac (SEQ ID NO: 268), CAGgugaggg (SEQ ID NO: 1343), UUGguaaaca (SEQ ID NO: 4157), AAGguagugu (SEQ ID NO: 4158), GAGguguggc (SEQ ID NO: 4159), CAGguacgga (SEQ ID NO: 4160), AAGgucauca (SEQ ID NO: 4161), CAAguaggca (SEQ ID NO: 4162), CAGgugaaac (SEQ ID NO: 4163), CAGguacugc (SEQ ID NO: 1192), AAUgcaagug (SEQ ID NO: 4164), CAUguaauuc (SEQ ID NO: 4165), AAGguaugcu (SEQ ID NO: 259), CUGgugaguu (SEQ ID NO: 1762), CAGgugguuu (SEQ ID NO: 4166), UGUgugagua (SEQ ID NO: 2922), AAGgucggug (SEQ ID NO: 4167), AUGguaaauu (SEQ ID NO: 883), AGGguauuac (SEQ ID NO: 771), AGUguaugga (SEQ ID NO: 4168), AACguaagau (SEQ ID NO: 4169), GUGguaaggu (SEQ ID NO: 4170), ACUguuagua (SEQ ID NO: 4171), CAGguaucag (SEQ ID NO: 1239), AAGguuaguu (SEQ ID NO: 425), CUGgugagcu (SEQ ID NO: 1754), UUGgugagcu (SEQ ID NO: 4172), UGUguacgua (SEQ ID NO: 4173), GAGgucagcc (SEQ ID NO: 4174), GAGguagaau (SEQ ID NO: 4175), AAGguaugag (SEQ ID NO: 255), UAGguauuuc (SEQ ID NO: 2563), UGUguaacac (SEQ ID NO: 4176), AGUguaaggc (SEQ ID NO: 4177), GAGgucugcu (SEQ ID NO: 4178), AAGguuagca (SEQ ID NO: 418), CAGguaaaug (SEQ ID NO: 1127), AACguaagcu (SEQ ID NO: 4179), CAGgucugca (SEQ ID NO: 4180), CAGguauugu (SEQ ID NO: 1267), GUGguaauuc (SEQ ID NO: 2356), GAGguauaug (SEQ ID NO: 1951), GCCgugagcc (SEQ ID NO: 4181), GAGguaagag (SEQ ID NO: 1883), UGAguaugua (SEQ ID NO: 2787), CAGguaaggg (SEQ ID NO: 1145), GAGguaaauu (SEQ ID NO: 1876), CAGgcaacuu (SEQ ID NO: 4182), UGUguaaguc (SEQ ID NO: 2908), CAGgugcgcu (SEQ ID NO: 4183), CGGguaaacc (SEQ ID NO: 4184), CCGgucaguc (SEQ ID NO: 4185), UAGgugggcg (SEQ ID NO: 4186), GCGgucaguu (SEQ ID NO: 4187), GGGguggguc (SEQ ID NO: 4188), AGCguaauag (SEQ ID NO: 4189), ACGgugaguc (SEQ ID NO: 4190), CUGguacuug (SEQ ID NO: 1722), CAGguuggua (SEQ ID NO: 4191), AGAguaugug (SEQ ID NO: 695), CUGgugggua (SEQ ID NO: 1771), GAGguggcuu (SEQ ID NO: 4192), AUAguauuga (SEQ ID NO: 4193), UGAgucguce (SEQ ID NO: 4194), CAGgugcucu (SEQ ID NO: 4195), UACguaauau (SEQ ID NO: 4196), GCUguccuga (SEQ ID NO: 4197), CAGgcugcac (SEQ ID NO: 4198), CUGgugcgcu (SEQ ID NO: 1766), GCGguaagaa (SEQ ID NO: 4199), UAAguuacuu (SEQ ID NO: 4200), GAAgugagug (SEQ ID NO: 1840), UAGgcaaguc (SEQ ID NO: 2460), UAAguaaaua (SEQ ID NO: 4201), ACGgugagug (SEQ ID NO: 607), CAGguagguu (SEQ ID NO: 1223), GGGguauaac (SEQ ID NO: 4202), GUUgugaguu (SEQ ID NO: 2410), CAUgugagua (SEQ ID NO: 1539), GAGgugcauu (SEQ ID NO: 4203), AAGguuugua (SEQ ID NO: 466), UCGguaaugu (SEQ ID NO: 4204), CGAguaaggg (SEQ ID NO: 1616), GAGgcacgga (SEQ ID NO: 4205), AGGgugugga (SEQ ID NO: 4206), CAGguauggu (SEQ ID NO: 1257), AAGguagaaa (SEQ ID NO: 203), CAGgugccug (SEQ ID NO: 1373), UGGguauaug (SEQ ID NO: 4207), UGAgugagac (SEQ ID NO: 4208), UGGguaauuu (SEQ ID NO: 2847), AUGguaaaua (SEQ ID NO: 881), AAGgcaaagg (SEQ ID NO: 4209), AGUguuuguu (SEQ ID NO: 4210), AUGguauugg (SEQ ID NO: 4211), CUGgugaggc (SEQ ID NO: 1756), UUGguaaaau (SEQ ID NO: 2948), ACAgugaguu (SEQ ID NO: 563), CAGgugcugu (SEQ ID NO: 4212), GAGguuaaga (SEQ ID NO: 2080), AGAguaagaa (SEQ ID NO: 659), GAGguccgcg (SEQ ID NO: 4213), GUGgugagga (SEQ ID NO: 2382), CAGgugagcc (SEQ ID NO: 1338), CAGgugacau (SEQ ID NO: 1324), AUGgcaagcu (SEQ ID NO: 4214), UCGguaauau (SEQ ID NO: 4215), CAGgcaacaa (SEQ ID NO: 4216), GGGguaggga (SEQ ID NO: 2257), CUGgucucgc (SEQ ID NO: 4217), UAGguaacga (SEQ ID NO: 4218), CGGguaaggu (SEQ ID NO: 4219), UAGguaaugc (SEQ ID NO: 4220), CAGgcaagaa (SEQ ID NO: 1099), ACAguaggua (SEQ ID NO: 4221), CAAguaugag (SEQ ID NO: 1049), GCUguucgaa (SEQ ID NO: 4222), AAGguuaugc (SEQ ID NO: 4223), GAUgugaguu (SEQ ID NO: 2136), CAGguggaga (SEQ ID NO: 1396), AGAguuaguu (SEQ ID NO: 4224), UGAgugugcg (SEQ ID NO: 4225), GAGguacagc (SEQ ID NO: 1907), CAGguaagac (SEQ ID NO: 1139), CAUgugcuuu (SEQ ID NO: 4226), AGGguguguu (SEQ ID NO: 4227), ACAguuaagg (SEQ ID NO: 4228), ACAgugaggg (SEQ ID NO: 4229), GAUguauacc (SEQ ID NO: 4230), UUAguaagcu (SEQ ID NO: 4231), CAGguaagau (SEQ ID NO: 1141), AGAgcugcgu (SEQ ID NO: 4232), GAGgcaaguu (SEQ ID NO: 1860), GAAguaagug (SEQ ID NO: 1819), AAGgugaaaa (SEQ ID NO: 4233), AAGguaccua (SEQ ID NO: 4234), GAGguaucag (SEQ ID NO: 4235), AUGguaugua (SEQ ID NO: 4236), AAGguaugaa (SEQ ID NO: 253), UUGgugagcc (SEQ ID NO: 4237), AAGguuagga (SEQ ID NO: 420), AGGguaugua (SEQ ID NO: 768), CAGguaccga (SEQ ID NO: 4238), AGAguaaacu (SEQ ID NO: 4239), AAGgugcaua (SEQ ID NO: 4240), AAGguaaugu (SEQ ID NO: 167), CCGgugugug (SEQ ID NO: 4241), AGGguaaauu (SEQ ID NO: 729), GGGguuuggc (SEQ ID NO: 4242), CAGguacacg (SEQ ID NO: 1164), UUGguaacca (SEQ ID NO: 4243), GAGgucaggu (SEQ ID NO: 1986), UCUguuggua (SEQ ID NO: 4244), CAGguuaguu (SEQ ID NO: 1458), UUGguauguc (SEQ ID NO: 4245), AAGgugcguc (SEQ ID NO: 4246), AGGguaagaa (SEQ ID NO: 733), UUUguaagcc (SEQ ID NO: 4247), AAGgucaggu (SEQ ID NO: 292), CUGguaaacu (SEQ ID NO: 4248), UCGguaauuu (SEQ ID NO: 4249), CUGguaggcu (SEQ ID NO: 4250), GAGgucugua (SEQ ID NO: 4251), GAGguacuuu (SEQ ID NO: 1922), CUGguaaagg (SEQ ID NO: 4252), CGGgugugug (SEQ ID NO: 1650), CAGguguggu (SEQ ID NO: 4253), UCGguacguc (SEQ ID NO: 4254), CAGgugccag (SEQ ID NO: 4255), GGGgugagaa (SEQ ID NO: 2275), ACAgcuagua (SEQ ID NO: 4256), AAGguauagc (SEQ ID NO: 4257), CUGguaggag (SEQ ID NO: 4258), GCUguacgua (SEQ ID NO: 4259), AAGguaaagg (SEQ ID NO: 128), CAAgcacgag (SEQ ID NO: 4260), CUAguaagac (SEQ ID NO: 4261), CCCguaagcg (SEQ ID NO: 4262), CAAgugugag (SEQ ID NO: 1078), AUGguaaggg (SEQ ID NO: 897), AAGgugaggg (SEQ ID NO: 345), CAAguaggua (SEQ ID NO: 1041), GGUguugcug (SEQ ID NO: 2321), GAGguacugu (SEQ ID NO: 1920), UAGguaagau (SEQ ID NO: 2484), CAGgugcgaa (SEQ ID NO: 1374), GAGguccagg (SEQ ID NO: 4263), UUGguauaca (SEQ ID NO: 2982), GGAgugagua (SEQ ID NO: 2226), GAGgugagau (SEQ ID NO: 2017), AAGguggggc (SEQ ID NO: 4264), CAGguaaacg (SEQ ID NO: 4265), UCGguaacuu (SEQ ID NO: 4266), CAGguaaauu (SEQ ID NO: 1128), GAGgugcgca (SEQ ID NO: 4267), ACUgugagua (SEQ ID NO: 643), ACGgugugac (SEQ ID NO: 4268), GUGguaaguc (SEQ ID NO: 2352), CAGguaggca (SEQ ID NO: 1215), CAGgucagca (SEQ ID NO: 1277), GUGguaugug (SEQ ID NO: 4269), AAAguaucug (SEQ ID NO: 4270), CGGguaugua (SEQ ID NO: 4271), AAGguaauaa (SEQ ID NO: 157), GAGgugggga (SEQ ID NO: 2060), GCUguaggug (SEQ ID NO: 2197), GAAgugaguu (SEQ ID NO: 1841), AAAguauuua (SEQ ID NO: 4272), UAUguaagua (SEQ ID NO: 2653), ACGguaugag (SEQ ID NO: 4273), CUGgugagug (SEQ ID NO: 1761), AGAguaaaau (SEQ ID NO: 4274), GCUguauggc (SEQ ID NO: 4275), AUGguaaacc (SEQ ID NO: 879), GCAguaauaa (SEQ ID NO: 4276), UAAguauuua (SEQ ID NO: 4277), AAUgucagug (SEQ ID NO: 515), AUUgcaggag (SEQ ID NO: 4278), CCGguaagaa (SEQ ID NO: 4279), AAGgcaaguu (SEQ ID NO: 101), GAGguuuguc (SEQ ID NO: 4280), AAGguaacug (SEQ ID NO: 139), AAAguaugag (SEQ ID NO: 4281), GAUguuagua (SEQ ID NO: 4282), CAGguggguc (SEQ ID NO: 1414), AAGguaccga (SEQ ID NO: 4283), CCAguaauua (SEQ ID NO: 4284), GUGguaugcg (SEQ ID NO: 4285), AUGgugcgcu (SEQ ID NO: 4286), CAGgucuaug (SEQ ID NO: 4287), AAGguauuua (SEQ ID NO: 274), CUAguaagau (SEQ ID NO: 4288), AGAguaauuu (SEQ ID NO: 675), GAGguaacgu (SEQ ID NO: 4289), AAGguagcca (SEQ ID NO: 212), CUGgucccgg (SEQ ID NO: 4290), GAGguccuuc (SEQ ID NO: 4291), ACGgucaccc (SEQ ID NO: 4292), AAGguaauac (SEQ ID NO: 158), CAGgugcaug (SEQ ID NO: 1367), AUGguaauag (SEQ ID NO: 4293), UUUguaacac (SEQ ID NO: 4294), UGGguaugau (SEQ ID NO: 4295), CAGgcccccc (SEQ ID NO: 4296), AGAguaguaa (SEQ ID NO: 4297), AGUguaagaa (SEQ ID NO: 814), GAAguauguu (SEQ ID NO: 1833), CAGgugugca (SEQ ID NO: 1434), UUGgugaggg (SEQ ID NO: 3003), UGGguugguu (SEQ ID NO: 4298), CAGguacgua (SEQ ID NO: 1184), GAGgugcggc (SEQ ID NO: 4299), UCUguacggg (SEQ ID NO: 4300), CGGgugcgug (SEQ ID NO: 4301), UACguaagug (SEQ ID NO: 2455), CAUguaagga (SEQ ID NO: 4302), CAGgugacgg (SEQ ID NO: 1329), GAUguaugcu (SEQ ID NO: 4303), UCUgcaauuc (SEQ ID NO: 4304), UGAguaaggc (SEQ ID NO: 2770), GAGguauauu (SEQ ID NO: 1952), AGAgugaguu (SEQ ID NO: 707), AAGguaagcu (SEQ ID NO: 148), UAGgugaagu (SEQ ID NO: 2580), CAGguuagua (SEQ ID NO: 1455), UAUguaagug (SEQ ID NO: 2655), UUGguggggg (SEQ ID NO: 4305), UGAgcucaaa (SEQ ID NO: 4306), UCGguaugua (SEQ ID NO: 4307), UAAguaugcc (SEQ ID NO: 4308), AAUguaagua (SEQ ID NO: 489), CAGguuugca (SEQ ID NO: 4309), ACGgugagag (SEQ ID NO: 4310), CAGguguuuu (SEQ ID NO: 4311), GUGgugagcc (SEQ ID NO: 4312), AGGguacaua (SEQ ID NO: 4313), UAGguaaccc (SEQ ID NO: 4314), GUGgucagua (SEQ ID NO: 4315), CUGgugagcc (SEQ ID NO: 4316), CAGgugcuua (SEQ ID NO: 1390), AUAgucguga (SEQ ID NO: 4317), AUAgugagug (SEQ ID NO: 862), GAGgucaaaa (SEQ ID NO: 4318), CGUguagcuu (SEQ ID NO: 4319), CAGguguuug (SEQ ID NO: 4320), CAGguuggac (SEQ ID NO: 4321), CAGguaagcu (SEQ ID NO: 4322), AGGgucagaa (SEQ ID NO: 4323), CACguauguc (SEQ ID NO: 4324), CACgugagug (SEQ ID NO: 1098), GGGguacgga (SEQ ID NO: 4325), AAGgcaggac (SEQ ID NO: 4326), GAGgugaagc (SEQ ID NO: 4327), GAGguuugaa (SEQ ID NO: 4328), CAGguaagug (SEQ ID NO: 1148), CAGguaacca (SEQ ID NO: 1131), CAGguacucc (SEQ ID NO: 1189), AAGgugcuuu (SEQ ID NO: 371), GAGguaaaua (SEQ ID NO: 1873), GAGgcaggug (SEQ ID NO: 4329), GAGguucgga (SEQ ID NO: 4330), CAGguauuug (SEQ ID NO: 1270), CAGguaaaua (SEQ ID NO: 1125), CAGgugaugu (SEQ ID NO: 1354), CAGgugauac (SEQ ID NO: 4331), GAGgugaggc (SEQ ID NO: 2023), AGGguggggg (SEQ ID NO: 4332), UAAguaaguu (SEQ ID NO: 2425), UGGgugaaca (SEQ ID NO: 4333), UAGguacugc (SEQ ID NO: 4334), CAGgcuccug (SEQ ID NO: 4335), AGGguaggca (SEQ ID NO: 753), CAGgugcccg (SEQ ID NO: 1371), GAGguacauc (SEQ ID NO: 4336), AGGgugugug (SEQ ID NO: 804), AAGguaguaa (SEQ ID NO: 231), UGGguaugag (SEQ ID NO: 2859), GGGgugugug (SEQ ID NO: 2294), CUAguaggug (SEQ ID NO: 4337), GAGgcaagga (SEQ ID NO: 4338), AAGgcaagac (SEQ ID NO: 4339), AAAgugcggu (SEQ ID NO: 4340), AAGguugguu (SEQ ID NO: 450), GAGguuaaug (SEQ ID NO: 4341), UUGgugaguc (SEQ ID NO: 3005), UCGguuagcu (SEQ ID NO: 2738), GCAguaagca (SEQ ID NO: 4342), AAGgcaagca (SEQ ID NO: 4343), ACAguaagcu (SEQ ID NO: 4344), GAGguaacag (SEQ ID NO: 1878), AAAguacgua (SEQ ID NO: 4345), GAGguaauac (SEQ ID NO: 1896), UUGguaggug (SEQ ID NO: 2980), CUGguuaguc (SEQ ID NO: 4346), GAGgugacgc (SEQ ID NO: 4347), ACAguaagga (SEQ ID NO: 4348), AAUguacuua (SEQ ID NO: 4349), GGGguacagu (SEQ ID NO: 4350), CGUguaugug (SEQ ID NO: 4351), UCCguagguu (SEQ ID NO: 4352), GAGguggucg (SEQ ID NO: 4353), UCAgugaguc (SEQ ID NO: 4354), AAAguaagca (SEQ ID NO: 15), GAGgucuggu (SEQ ID NO: 1999), GAGguaauua (SEQ ID NO: 4355), GUAguaagua (SEQ ID NO: 2323), AAGgugggga (SEQ ID NO: 382), UCUgugagca (SEQ ID NO: 4356), GAAguucgug (SEQ ID NO: 4357), ACGgugaggc (SEQ ID NO: 4358), UCAgugagua (SEQ ID NO: 2699), UAGguaguug (SEQ ID NO: 4359), GGUgucuggg (SEQ ID NO: 4360), GGGguaagug (SEQ ID NO: 2252), GAGguggguu (SEQ ID NO: 2066), UGUgugaguu (SEQ ID NO: 4361), CAUguaagua (SEQ ID NO: 1522), AAGguaggug (SEQ ID NO: 229), AAUguaggag (SEQ ID NO: 4362), GAGgcacguc (SEQ ID NO: 4363), CAAguacauu (SEQ ID NO: 4364), UUGguacaga (SEQ ID NO: 4365), GAGguaguag (SEQ ID NO: 1941), AAAgugaggg (SEQ ID NO: 57), UUGgucagug (SEQ ID NO: 4366), AGGgugaguc (SEQ ID NO: 796), CAGgugaaca (SEQ ID NO: 1317), GGUgugggcc (SEQ ID NO: 4367), CGGgugagcu (SEQ ID NO: 4368), GGGgugaguc (SEQ ID NO: 2283), ACAgugagag (SEQ ID NO: 4369), AGGgugaggu (SEQ ID NO: 794), GCUguaaguc (SEQ ID NO: 2194), AUAguagguu (SEQ ID NO: 4370), CAGgcaugug (SEQ ID NO: 1114), AAGguaaguu (SEQ ID NO: 156), CAGguccgug (SEQ ID NO: 4371), GAGgcaggua (SEQ ID NO: 4372), AUGguggaag (SEQ ID NO: 4373), AUGgugggcg (SEQ ID NO: 4374), GAGgugagaa (SEQ ID NO: 2014), AGUgugagca (SEQ ID NO: 832), UUGguaagua (SEQ ID NO: 2962), CAAguaagca (SEQ ID NO: 4375), GGUgugagcu (SEQ ID NO: 2313), CCCgugggua (SEQ ID NO: 4376), CAGguagaau (SEQ ID NO: 4377), CAGgcugagc (SEQ ID NO: 4378), CUGguggece (SEQ ID NO: 4379), UGAguaagag (SEQ ID NO: 4380), CACguuagcu (SEQ ID NO: 4381), AAGgugaguc (SEQ ID NO: 348), AAGguagcuc (SEQ ID NO: 4382), UCGgugaguu (SEQ ID NO: 4383), GAGgcccuuc (SEQ ID NO: 4384), CAGguuaugc (SEQ ID NO: 4385), CCUguaagcu (SEQ ID NO: 4386), CAGgucuccu (SEQ ID NO: 4387), UAGguaggcu (SEQ ID NO: 4388), GGGguagggg (SEQ ID NO: 4389), AAGguaguga (SEQ ID NO: 4390), GAGguuguug (SEQ ID NO: 4391), CAGguugguu (SEQ ID NO: 1489), AAAguaagcc (SEQ ID NO: 16), ACAgugagug (SEQ ID NO: 562), UGGgugugau (SEQ ID NO: 4392), CCCguaacua (SEQ ID NO: 4393), AAGguguugc (SEQ ID NO: 408), AAAgcuggug (SEQ ID NO: 4394), GAGguauagu (SEQ ID NO: 4395), ACGguaagag (SEQ ID NO: 4396), AUGguacggu (SEQ ID NO: 913), GAGgccaguu (SEQ ID NO: 4397), GAGguaugcg (SEQ ID NO: 1960), UCGgugggag (SEQ ID NO: 4398), AAGguggaua (SEQ ID NO: 372), CCAguguggc (SEQ ID NO: 4399), AGGguaagug (SEQ ID NO: 742), UCUguagguc (SEQ ID NO: 4400), CAGgcaagga (SEQ ID NO: 1102), CGGguaauuu (SEQ ID NO: 1628), AUUgugaguc (SEQ ID NO: 1010), CAGguaaacc (SEQ ID NO: 1121), AAGgucaauu (SEQ ID NO: 4401), AAGgugaaua (SEQ ID NO: 327), GUCguaagaa (SEQ ID NO: 4402), GCGguaaguc (SEQ ID NO: 4403), CUGguagagc (SEQ ID NO: 4404), GAGgucgguc (SEQ ID NO: 4405), CAGguaaaca (SEQ ID NO: 1120), AAGgcaagga (SEQ ID NO: 98), CAGgucgucu (SEQ ID NO: 4406), GGGguagggc (SEQ ID NO: 4407), CUGguacuaa (SEQ ID NO: 1721), GAGguagcug (SEQ ID NO: 1929), CUUgucagcu (SEQ ID NO: 4408), UAGguaaggc (SEQ ID NO: 2489), CUGguauuac (SEQ ID NO: 4409), UAAguacguc (SEQ ID NO: 4410), AAGguaagcc (SEQ ID NO: 146), ACGgugaaag (SEQ ID NO: 4411), CCAgccaaua (SEQ ID NO: 4412), CAGguuuguc (SEQ ID NO: 4413), AAGguauaau (SEQ ID NO: 239), AAGgucuuag (SEQ ID NO: 4414), AGGgugagcu (SEQ ID NO: 791), AAGguuaggg (SEQ ID NO: 4415), CGGguaaauu (SEQ ID NO: 4416), CAGguaacgg (SEQ ID NO: 4417), AGAgugugua (SEQ ID NO: 4418), ACAguaaguu (SEQ ID NO: 549), GAUguaauuu (SEQ ID NO: 4419), GAGguaggga (SEQ ID NO: 1934), UUGgcaagug (SEQ ID NO: 2945), AAAgugagga (SEQ ID NO: 4420), AAGguagugc (SEQ ID NO: 234), AGAguaauuc (SEQ ID NO: 674), GGAguaaaua (SEQ ID NO: 4421), GUGguaccca (SEQ ID NO: 4422), CAGguauugc (SEQ ID NO: 4423), GAUgugaggg (SEQ ID NO: 4424), CAAguaaauc (SEQ ID NO: 1017), CAGgugucuc (SEQ ID NO: 1428), AAGguaacag (SEQ ID NO: 4425), UUGguaaaag (SEQ ID NO: 4426), CAGguaucau (SEQ ID NO: 1240), ACGgugagac (SEQ ID NO: 4427), CUGguaugac (SEQ ID NO: 4428), CAGguucacu (SEQ ID NO: 4429), GAGgugauca (SEQ ID NO: 4430), AGUguaaguc (SEQ ID NO: 4431), AACguaagua (SEQ ID NO: 4432), AAAgugagug (SEQ ID NO: 60), GAGguacagg (SEQ ID NO: 4433), CAAguaauga (SEQ ID NO: 4434), GAUguaagga (SEQ ID NO: 4435), UCAguucccc (SEQ ID NO: 4436), GCGguaagga (SEQ ID NO: 4437), UAGguacuaa (SEQ ID NO: 4438), AAGgugaaag (SEQ ID NO: 321), ACUguaagug (SEQ ID NO: 4439), UGGguaugug (SEQ ID NO: 2862), AUGguaacag (SEQ ID NO: 884), CAGguagggu (SEQ ID NO: 1219), ACAguaagug (SEQ ID NO: 548), AAGgugcucc (SEQ ID NO: 366), AAGgugugcu (SEQ ID NO: 4440), AAGgugguga (SEQ ID NO: 4441), ACGgugcgcc (SEQ ID NO: 4442), AAGguauugc (SEQ ID NO: 4443), GGGguaugug (SEQ ID NO: 2267), CAGgugggcu (SEQ ID NO: 1408), GAGguauguu (SEQ ID NO: 1968), AACgugaaua (SEQ ID NO: 4444), CAGguaaugg (SEQ ID NO: 1154), UAGguaugau (SEQ ID NO: 4445), CAGgcaggug (SEQ ID NO: 1108), GGGguugguc (SEQ ID NO: 4446), AAGguauggg (SEQ ID NO: 262), UAAgugaggc (SEQ ID NO: 4447), CAAgugaucg (SEQ ID NO: 4448), AAAguacggg (SEQ ID NO: 4449), AGAgcuacag (SEQ ID NO: 4450), GAGgugggaa (SEQ ID NO: 2054), CAGguacuuu (SEQ ID NO: 1195), GAGgugagag (SEQ ID NO: 2016), CAGguagguc (SEQ ID NO: 1221), UGGguacagc (SEQ ID NO: 4451), AAGgugucag (SEQ ID NO: 396), AAGgcaagaa (SEQ ID NO: 4452), GAGguaaaca (SEQ ID NO: 4453), AAGguaaagu (SEQ ID NO: 129), AAGguaguca (SEQ ID NO: 4454), CUGguauguc (SEQ ID NO: 4455), GAGguauggg (SEQ ID NO: 1963), AAGguauugu (SEQ ID NO: 273), CUGguacuga (SEQ ID NO: 4456), GAGguaagcu (SEQ ID NO: 1888), UGGgugggua (SEQ ID NO: 2883), CAGguucgug (SEQ ID NO: 4457), AAGguauggu (SEQ ID NO: 4458), CAGgugagca (SEQ ID NO: 1337), UGGguaaauu (SEQ ID NO: 2827), UGUguaggug (SEQ ID NO: 4459), UGUgugagcc (SEQ ID NO: 2921), CUGguaauau (SEQ ID NO: 4460), AAAguauguu (SEQ ID NO: 45), UGUguaagaa (SEQ ID NO: 2903), CUAgugagaa (SEQ ID NO: 4461), AGGguagguc (SEQ ID NO: 757), AAGgugggug (SEQ ID NO: 385), UCGguaagug (SEQ ID NO: 4462), AGUguaaaua (SEQ ID NO: 812), GAUguaagug (SEQ ID NO: 2122), AAGguuagug (SEQ ID NO: 424), UAGguaagca (SEQ ID NO: 2485), CAAgugagaa (SEQ ID NO: 1061), AGUguaagua (SEQ ID NO: 819), CAGgugaauc (SEQ ID NO: 1321), UGGgugagac (SEQ ID NO: 2868), AAGguagggc (SEQ ID NO: 224), CUGguuugug (SEQ ID NO: 1788), GCGguagggc (SEQ ID NO: 4463), GAGguaaucc (SEQ ID NO: 4464), AUUguaauaa (SEQ ID NO: 4465), CUGgugaaua (SEQ ID NO: 1748), AAGguuuaaa (SEQ ID NO: 4466), CCUguacugu (SEQ ID NO: 4467), GCGgugagcg (SEQ ID NO: 4468), AAGguaaucc (SEQ ID NO: 162), UAUgugagua (SEQ ID NO: 2671), CCCgugagug (SEQ ID NO: 1573), CAGgugcaga (SEQ ID NO: 1363), CAGgucaguu (SEQ ID NO: 1284), CAGguaggcu (SEQ ID NO: 4469), AAAguaagug (SEQ ID NO: 23), UAGguugguc (SEQ ID NO: 4470), CAGguugccu (SEQ ID NO: 4471), AAGguaugga (SEQ ID NO: 260), GGUguggacg (SEQ ID NO: 4472), AAAgugagaa (SEQ ID NO: 51), AGGgugagag (SEQ ID NO: 788), GAUguggcau (SEQ ID NO: 4473), UCGguaaggu (SEQ ID NO: 4474), GAGgugcguc (SEQ ID NO: 4475), CGGgugaguc (SEQ ID NO: 4476), AAGguacggg (SEQ ID NO: 190), GAGguucuug (SEQ ID NO: 4477), AAGgugcuug (SEQ ID NO: 4478), UAGguaugua (SEQ ID NO: 2551), AUGgucagca (SEQ ID NO: 4479), CGGguacuca (SEQ ID NO: 4480), AGGgugagga (SEQ ID NO: 792), AUCgugagua (SEQ ID NO: 869), UCAguaagua (SEQ ID NO: 2689), UAGguaaaua (SEQ ID NO: 2469), AAGguaauug (SEQ ID NO: 170), GAAgucagug (SEQ ID NO: 1835), CAGguacaaa (SEQ ID NO: 1160), AAAguuaauc (SEQ ID NO: 4481), AGCgugagcg (SEQ ID NO: 4482), CCGgcuggug (SEQ ID NO: 4483), AGUguaauuu (SEQ ID NO: 4484), UGAgccacuc (SEQ ID NO: 4485), GGGgucugua (SEQ ID NO: 4486), AUGgcauguc (SEQ ID NO: 4487), CGGguaaaga (SEQ ID NO: 4488), AGGguagcau (SEQ ID NO: 4489), CGGguaggag (SEQ ID NO: 1631), GAGguucgug (SEQ ID NO: 4490), UAAguuauuc (SEQ ID NO: 4491), UAUguaagau (SEQ ID NO: 2650), AAGguaguuu (SEQ ID NO: 237), CAGgugguau (SEQ ID NO: 4492), GUGguaauga (SEQ ID NO: 2355), AAGgugauuu (SEQ ID NO: 359), CAGgugaagu (SEQ ID NO: 4493), GUAguaauua (SEQ ID NO: 4494), AUGguuggug (SEQ ID NO: 4495), CCAguaagug (SEQ ID NO: 1557), UAGgugagag (SEQ ID NO: 2589), AUGgugaggc (SEQ ID NO: 959), AAAguuagug (SEQ ID NO: 72), AAGgugccuu (SEQ ID NO: 4496), UAGguaugag (SEQ ID NO: 2546), CAGgugugac (SEQ ID NO: 1431), CUGguggguu (SEQ ID NO: 1774), AUGguaagga (SEQ ID NO: 896), UCUguaagaa (SEQ ID NO: 2740), UCCgugaguu (SEQ ID NO: 4497), AAAgcaggua (SEQ ID NO: 4498), UAUgugagug (SEQ ID NO: 2672), CAGguggagg (SEQ ID NO: 4499), CAGguuagac (SEQ ID NO: 4500), AUAguaagac (SEQ ID NO: 846), AAGguguugu (SEQ ID NO: 4501), GAGgucugug (SEQ ID NO: 4502), AAGguaagau (SEQ ID NO: 144), CAUguaaguu (SEQ ID NO: 1524), CUGguaauua (SEQ ID NO: 4503), CAGguaggcg (SEQ ID NO: 4504), AGAguaaguc (SEQ ID NO: 669), UGGgugagga (SEQ ID NO: 2872), AAUguaggua (SEQ ID NO: 4505), UAGguuagca (SEQ ID NO: 4506), GGGguaggua (SEQ ID NO: 2258), GAGguauugc (SEQ ID NO: 4507), AUUguacaca (SEQ ID NO: 4508), GAAguaggua (SEQ ID NO: 4509), GGAguaagcu (SEQ ID NO: 2212), UAGguaugug (SEQ ID NO: 2553), GAGgugaaua (SEQ ID NO: 2007), GAGgugggau (SEQ ID NO: 2056), AAGguaaucu (SEQ ID NO: 163), GGUgugaguu (SEQ ID NO: 4510), AACgugaguu (SEQ ID NO: 4511), GAGguaaccg (SEQ ID NO: 4512), UAGguaagga (SEQ ID NO: 2488), AUUguaagaa (SEQ ID NO: 4513), UGGgugagca (SEQ ID NO: 2870), AAGguaaggc (SEQ ID NO: 150), CCAguaucgu (SEQ ID NO: 4514), CCGgugggug (SEQ ID NO: 4515), GAGguagugu (SEQ ID NO: 4516), ACGgugggaa (SEQ ID NO: 4517), GAGgugaccu (SEQ ID NO: 2011), CACguaugua (SEQ ID NO: 4518), AGGgugggga (SEQ ID NO: 799), AAUguaaguc (SEQ ID NO: 490), AAAguuaagu (SEQ ID NO: 70), CAUgugagug (SEQ ID NO: 1541), AGAguauguc (SEQ ID NO: 694), GCGguaugac (SEQ ID NO: 4519), CGGgugaguu (SEQ ID NO: 1643), CCGguauuuu (SEQ ID NO: 4520), GAGguagaac (SEQ ID NO: 4521), UAGguaugaa (SEQ ID NO: 2545), CAGgcgcgug (SEQ ID NO: 4522), CAAguaaguc (SEQ ID NO: 1027), AGUguaagau (SEQ ID NO: 816), AAGguucuac (SEQ ID NO: 4523), CCAguaagua (SEQ ID NO: 1555), GAGguagcag (SEQ ID NO: 4524), CAGgucuguu (SEQ ID NO: 1312), CAGguacaau (SEQ ID NO: 1162), CCGguaaaga (SEQ ID NO: 1574), UAAgugcugu (SEQ ID NO: 4525), AGGgugagaa (SEQ ID NO: 786), CUCguaaggu (SEQ ID NO: 4526), CAGgucagcu (SEQ ID NO: 4527), CAGguaaggc (SEQ ID NO: 1144), AGGgugcagg (SEQ ID NO: 4528), GAGgugaaac (SEQ ID NO: 4529), AGGguaagua (SEQ ID NO: 740), AAUguaugcc (SEQ ID NO: 4530), AAGguaagca (SEQ ID NO: 145), ACGguacggu (SEQ ID NO: 587), AAGguaauga (SEQ ID NO: 164), UCUgcucaau (SEQ ID NO: 4531), ACGguaaugu (SEQ ID NO: 4532), AAGguaguug (SEQ ID NO: 4533), ACGguaagug (SEQ ID NO: 580), CAGgugauga (SEQ ID NO: 4534), GAGguaacac (SEQ ID NO: 4535), GAGguaggua (SEQ ID NO: 1937), CAGguaccuu (SEQ ID NO: 1179), CAGguaauaa (SEQ ID NO: 1150), UUGgugggug (SEQ ID NO: 3016), CUGguaauga (SEQ ID NO: 1710), UAGguaaguc (SEQ ID NO: 2492), AGGgugugac (SEQ ID NO: 4536), GAGgcaauaa (SEQ ID NO: 4537), GUGguaaagc (SEQ ID NO: 4538), CUGgugggcg (SEQ ID NO: 4539), GAUguauguu (SEQ ID NO: 2128), AGGgugagac (SEQ ID NO: 787), UCGgucagca (SEQ ID NO: 4540), AUGgugauua (SEQ ID NO: 4541), CGAgugugua (SEQ ID NO: 4542), CAGguuggug (SEQ ID NO: 1488), AGCgcaagua (SEQ ID NO: 4543), UGGguacguu (SEQ ID NO: 4544), GAGguauuug (SEQ ID NO: 1974), AGUguacaua (SEQ ID NO: 4545), AUGguaagua (SEQ ID NO: 898), ACAguagguu (SEQ ID NO: 4546), AAGgugagag (SEQ ID NO: 337), UUGgugaagu (SEQ ID NO: 4547), AAAguaugua (SEQ ID NO: 43), UGGguaagga (SEQ ID NO: 4548), UAGgugccuu (SEQ ID NO: 4549), and CCUgugggug (SEQ ID NO: 4550). Additional exemplary gene sequences and splice site sequences (e.g., 5′ splice site sequences) include UCCguaaguu (SEQ ID NO: 4551), GUGguaaacg (SEQ ID NO: 4552), CGGgugcggu (SEQ ID NO: 4553), CAUguacuuc (SEQ ID NO: 4554), AGAguaaagg (SEQ ID NO: 4555), CGCgugagua (SEQ ID NO: 4556), AGAgugggca (SEQ ID NO: 4557), AGAguaagcc (SEQ ID NO: 4558), AGAguaaaca (SEQ ID NO: 4559), GUGguuauga (SEQ ID NO: 4560), AGGguaauaa (SEQ ID NO: 4561), UGAguaagac (SEQ ID NO: 4562), AGAguuuguu (SEQ ID NO: 4563), CGGgucugca (SEQ ID NO: 4564), CAGguaaguc (SEQ ID NO: 4565), AAGguagaau (SEQ ID NO: 4566), CAGgucccuc (SEQ ID NO: 4567), AGAguaaugg (SEQ ID NO: 4568), GAGgucuaag (SEQ ID NO: 4569), AGAguagagu (SEQ ID NO: 4570), AUGgucagua (SEQ ID NO: 4571), GAGgccuggg (SEQ ID NO: 4572), AAGguguggc (SEQ ID NO: 4573), AGAgugaucu (SEQ ID NO: 4574), AAGguaucca (SEQ ID NO: 4575), UUCguaagua (SEQ ID NO: 4576), UAAgugggug (SEQ ID NO: 4577), GCCgugaacg (SEQ ID NO: 4578), GAGguugugg (SEQ ID NO: 4579), UAUguaugca (SEQ ID NO: 4580), UGUguaacaa (SEQ ID NO: 4581), AGGguauuag (SEQ ID NO: 4582), UGAguauauc (SEQ ID NO: 4583), AGAguuugug (SEQ ID NO: 4584), GAGgucgcug (SEQ ID NO: 4585), GAGgucaucg (SEQ ID NO: 4586), ACGguaaagc (SEQ ID NO: 4587), UGAguacuug (SEQ ID NO: 4588), CGAgucgccg (SEQ ID NO: 4589), CUGguacguc (SEQ ID NO: 4590), AGGguauugc (SEQ ID NO: 4591), GAAgugaaug (SEQ ID NO: 4592), CAGaugaguc (SEQ ID NO: 4593), UGGguauugg (SEQ ID NO: 4594), UGAguaaaga (SEQ ID NO: 4595), GUGguuccug (SEQ ID NO: 4596), UGAgcaagua (SEQ ID NO: 4597), UAUguaagag (SEQ ID NO: 4598), AAGgucuugc (SEQ ID NO: 4599), AAAgcaugug (SEQ ID NO: 4600), AGAguacagu (SEQ ID NO: 4601), GUGguaaucc (SEQ ID NO: 4602), CAGguagagg (SEQ ID NO: 4603), AAGguacaac (SEQ ID NO: 4604), UGGgcagcau (SEQ ID NO: 4605), CCGgucauca (SEQ ID NO: 4606), CCGguuugua (SEQ ID NO: 4607), UGAguaaggg (SEQ ID NO: 4608), GAAguaugua (SEQ ID NO: 4609), GGGguagcuc (SEQ ID NO: 4610), GCUguacaua (SEQ ID NO: 4611), CUGgucucuu (SEQ ID NO: 4612), GUGguaaaug (SEQ ID NO: 4613), AUCguaagug (SEQ ID NO: 4614), GAGgcaugua (SEQ ID NO: 4615), AAGgucuccc (SEQ ID NO: 4616), UGGgugcguu (SEQ ID NO: 4617), UGUguagguu (SEQ ID NO: 4618), GAAgugagca (SEQ ID NO: 4619), GGUguaauuu (SEQ ID NO: 4620), CUGgugaaau (SEQ ID NO: 4621), AUCguaaguc (SEQ ID NO: 4622), AGAguaaucc (SEQ ID NO: 4623), GGAguagguc (SEQ ID NO: 4624), GAGguaccaa (SEQ ID NO: 4625), CUUguaggug (SEQ ID NO: 4626), AAGguauaag (SEQ ID NO: 4627), AGAguuggua (SEQ ID NO: 4628), AUGguuugug (SEQ ID NO: 4629), UGGgucagau (SEQ ID NO: 4630), AGAguaggac (SEQ ID NO: 4631), AGAguagugu (SEQ ID NO: 4632), AGAguaggag (SEQ ID NO: 4633), CAGgucucua (SEQ ID NO: 4634), AAGguggaug (SEQ ID NO: 4635), UGGguaucaa (SEQ ID NO: 4636), GAUguaugga (SEQ ID NO: 4637), AAGguguuuc (SEQ ID NO: 4638), GCAguguaaa (SEQ ID NO: 4639), UUAguaugua (SEQ ID NO: 4640), UCUguaugca (SEQ ID NO: 4641), AAUguaaaau (SEQ ID NO: 4642), AGAguaaauu (SEQ ID NO: 4643), GGGguacuuu (SEQ ID NO: 4644), GAAguuugau (SEQ ID NO: 4645), AAAguagauu (SEQ ID NO: 4646), UGUguagagu (SEQ ID NO: 4647), UGGguaagcg (SEQ ID NO: 4648), CGGguucagg (SEQ ID NO: 4649), AGGguacgac (SEQ ID NO: 4650), UCGguaagaa (SEQ ID NO: 4651), AGGguuggca (SEQ ID NO: 4652), AAAguacagu (SEQ ID NO: 4653), UAAguuaagg (SEQ ID NO: 4654), AUGguaaugu (SEQ ID NO: 4655), GUGguuuuac (SEQ ID NO: 4656), AGAguaacaa (SEQ ID NO: 4657), AAGguagccc (SEQ ID NO: 4658), GCGgugaggc (SEQ ID NO: 4659), AUGguucagc (SEQ ID NO: 4660), AAGguacuua (SEQ ID NO: 4661), AAGguccgug (SEQ ID NO: 4662), UAGguaagcg (SEQ ID NO: 4663), AUGguaccuu (SEQ ID NO: 4664), GCCguggugg (SEQ ID NO: 4665), CUGgugeguc (SEQ ID NO: 4666), CAGguggaaa (SEQ ID NO: 4667), AAAgucugua (SEQ ID NO: 4668), GAGguaaccc (SEQ ID NO: 4669), AGAguauggg (SEQ ID NO: 4670), UAUgccccug (SEQ ID NO: 4671), AAGgugccag (SEQ ID NO: 4672), ACGgugcggc (SEQ ID NO: 4673), AGGguacuga (SEQ ID NO: 4674), AGAguaagcg (SEQ ID NO: 4675), CUGgcaaggg (SEQ ID NO: 4676), CCAgugugug (SEQ ID NO: 4677), GAGguagacg (SEQ ID NO: 4678), CGGgugcggg (SEQ ID NO: 4679), GAUguaagcu (SEQ ID NO: 4680), AUUguauuua (SEQ ID NO: 4681), UGCgugagug (SEQ ID NO: 4682), CUGgucuaua (SEQ ID NO: 4683), GAGgugcuag (SEQ ID NO: 4684), GAGgugccau (SEQ ID NO: 4685), CAGguacguc (SEQ ID NO: 4686), GAGguucagc (SEQ ID NO: 4687), AACguaagaa (SEQ ID NO: 4688), AGAguaguac (SEQ ID NO: 4689), AAGguaacgg (SEQ ID NO: 4690), UAGgugugac (SEQ ID NO: 4691), CCGguaauag (SEQ ID NO: 4692), CAGguaccag (SEQ ID NO: 4693), UUUguaauug (SEQ ID NO: 4694), AAUguacgaa (SEQ ID NO: 4695), CAGguaauga (SEQ ID NO: 4696), AUCgucaagg (SEQ ID NO: 4697), CUGguagaug (SEQ ID NO: 4698), GGGgugcagu (SEQ ID NO: 4699), AGUgugagaa (SEQ ID NO: 4700), GGGguuuuau (SEQ ID NO: 4701), CCUguccccu (SEQ ID NO: 4702), AUUgugaagu (SEQ ID NO: 4703), AAGguaaacg (SEQ ID NO: 4704), UACgucgugg (SEQ ID NO: 4705), AAGgugccau (SEQ ID NO: 4706), GGGgucccag (SEQ ID NO: 4707), UAUguauggu (SEQ ID NO: 4708), CGGguaauua (SEQ ID NO: 4709), CGGguacucc (SEQ ID NO: 4710), CAGgugacuu (SEQ ID NO: 4711), AGUguggguu (SEQ ID NO: 4712), AGAguauggc (SEQ ID NO: 4713), AAGgccaaca (SEQ ID NO: 4714), AAAgcaagua (SEQ ID NO: 4715), UCAguagguc (SEQ ID NO: 4716), GUGguggcgg (SEQ ID NO: 4717), CAUguauccu (SEQ ID NO: 4718), UCGgugagcc (SEQ ID NO: 4719), AUAguugggu (SEQ ID NO: 4720), AAUguuagcu (SEQ ID NO: 4721), AUGgugaaug (SEQ ID NO: 4722), CGGguaaugu (SEQ ID NO: 4723), UCUguaggug (SEQ ID NO: 4724), CCGgugaggc (SEQ ID NO: 4725), UGAguccacu (SEQ ID NO: 4726), CUAguaagag (SEQ ID NO: 4727), CGGguggggc (SEQ ID NO: 4728), CGAguaagca (SEQ ID NO: 4729), UGUgccaauu (SEQ ID NO: 4730), UCGguaagcc (SEQ ID NO: 4731), UAUguaggug (SEQ ID NO: 4732), UUGgugggcc (SEQ ID NO: 4733), GAGgcugggc (SEQ ID NO: 4734), AGAguaacuu (SEQ ID NO: 4735), ACGguagguc (SEQ ID NO: 4736), CAGgcccaga (SEQ ID NO: 4737), CCGguggguu (SEQ ID NO: 4738), AAGgugacgg (SEQ ID NO: 4739), GGGguacagc (SEQ ID NO: 4740), CAUguaaguc (SEQ ID NO: 4741), AUUgugagaa (SEQ ID NO: 4742), UGUguaagga (SEQ ID NO: 4743), UUUguaagau (SEQ ID NO: 4744), AGGgucauuu (SEQ ID NO: 4745), UGGguuuguu (SEQ ID NO: 4746), CGAguaagcc (SEQ ID NO: 4747), GUGgugugua (SEQ ID NO: 4748), AUGguauaac (SEQ ID NO: 4749), UGGguacgua (SEQ ID NO: 4750), AAAguagagu (SEQ ID NO: 4751), UCGguaacug (SEQ ID NO: 4752), AGAguaauga (SEQ ID NO: 4753), AUGguggguc (SEQ ID NO: 4754), AGAguaauau (SEQ ID NO: 4755), CAGguacugg (SEQ ID NO: 4756), UAAgucaguu (SEQ ID NO: 4757), GCGguagaga (SEQ ID NO: 4758), AAGgugaugg (SEQ ID NO: 4759), ACAguauguu (SEQ ID NO: 4760), GAUguacguc (SEQ ID NO: 4761), UAGguuucuc (SEQ ID NO: 4762), GAGgcauggg (SEQ ID NO: 4763), AUAgcuaagu (SEQ ID NO: 4764), GUAgucugua (SEQ ID NO: 4765), AAGgugaacg (SEQ ID NO: 4766), GUGguggucg (SEQ ID NO: 4767), GAGguugauc (SEQ ID NO: 4768), UGAguggguu (SEQ ID NO: 4769), ACUguacgug (SEQ ID NO: 4770), CUGgugacug (SEQ ID NO: 4771), CAAguuaagc (SEQ ID NO: 4772), GAGguaccca (SEQ ID NO: 4773), AACguaacuu (SEQ ID NO: 4774), CAGguuacua (SEQ ID NO: 4775), AGAguuaguc (SEQ ID NO: 4776), UGGgcacguc (SEQ ID NO: 4777), AGUguauggu (SEQ ID NO: 4778), AAGguugcaa (SEQ ID NO: 4779), CAGguuguua (SEQ ID NO: 4780), AAGgcauccc (SEQ ID NO: 4781), GAUguaaggc (SEQ ID NO: 4782), AGGguacggg (SEQ ID NO: 4783), GAGgucaaag (SEQ ID NO: 4784), CAAgugagcg (SEQ ID NO: 4785), AGAguaaucu (SEQ ID NO: 4786), UCGguagcug (SEQ ID NO: 4787), AAAguaguag (SEQ ID NO: 4788), CAGguucguc (SEQ ID NO: 4789), CGUguaugaa (SEQ ID NO: 4790), AGUguaaaaa (SEQ ID NO: 4791), AAGgucucac (SEQ ID NO: 4792), UAGguggagc (SEQ ID NO: 4793), UGAguaggug (SEQ ID NO: 4794), AGAguaugcc (SEQ ID NO: 4795), GAGguugcau (SEQ ID NO: 4796), CAAguaagag (SEQ ID NO: 4797), UCUgugugcc (SEQ ID NO: 4798), GAGgugaugc (SEQ ID NO: 4799), GGGgugauaa (SEQ ID NO: 4800), CCCgugagcc (SEQ ID NO: 4801), AGAguaacug (SEQ ID NO: 4802), GCGguaagua (SEQ ID NO: 4803), AGAguacauc (SEQ ID NO: 4804), UCGgucuggg (SEQ ID NO: 4805), UAAguaucuc (SEQ ID NO: 4806), GGCguagguu (SEQ ID NO: 4807), AGAguacgcc (SEQ ID NO: 4808), GAUgucuucu (SEQ ID NO: 4809), AGGgcaaggu (SEQ ID NO: 4810), CGAguaugau (SEQ ID NO: 4811), AUGguagagu (SEQ ID NO: 4812), CAAguacgag (SEQ ID NO: 4813), UCGguaugau (SEQ ID NO: 4814), CCGguguguu (SEQ ID NO: 4815), AGGgucugug (SEQ ID NO: 4816), GGAguaggcu (SEQ ID NO: 4817), AAGgucuaug (SEQ ID NO: 4818), GCAgugcgug (SEQ ID NO: 4819), UGGgugagaa (SEQ ID NO: 4820), AGGguaaagu (SEQ ID NO: 4821), GAGguaggac (SEQ ID NO: 4822), CUAguaagca (SEQ ID NO: 4823), UUAguaggcu (SEQ ID NO: 4824), CUGgugggau (SEQ ID NO: 4825), CUGguuagua (SEQ ID NO: 4826), AAGguacgug (SEQ ID NO: 4827), CGGgugagau (SEQ ID NO: 4828), AAGgugcaug (SEQ ID NO: 4829), AAUgugggcu (SEQ ID NO: 4830), CAGguugacu (SEQ ID NO: 4831), CAGguuacag (SEQ ID NO: 4832), GCGguaacau (SEQ ID NO: 4833), AUUgucaguc (SEQ ID NO: 4834), CAAguauaca (SEQ ID NO: 4835), GAUguccgcc (SEQ ID NO: 4836), AAGgugcgga (SEQ ID NO: 4837), AACguaagag (SEQ ID NO: 4838), UGGguuggua (SEQ ID NO: 4839), CAAguguaag (SEQ ID NO: 4840), GUGguaacgu (SEQ ID NO: 4841), CUGgugauca (SEQ ID NO: 4842), AGGguggggc (SEQ ID NO: 4843), UCGguaaaga (SEQ ID NO: 4844), CAGguacacc (SEQ ID NO: 4845), CGGguaaggg (SEQ ID NO: 4846), CAAguuugcu (SEQ ID NO: 4847), ACAgugcgug (SEQ ID NO: 4848), UUGguauggg (SEQ ID NO: 4849), GAGgcucauc (SEQ ID NO: 4850), CUGguaauag (SEQ ID NO: 4851), AUGguggaua (SEQ ID NO: 4852), UCAgugaauu (SEQ ID NO: 4853), AAUguaauua (SEQ ID NO: 4854), GCAgucuaaa (SEQ ID NO: 4855), AAGguauucu (SEQ ID NO: 4856), GAGgucauca (SEQ ID NO: 4857), UGGguccaug (SEQ ID NO: 4858), AGAguuugua (SEQ ID NO: 4859), AGGguagacu (SEQ ID NO: 4860), AAGguaggac (SEQ ID NO: 4861), UGUguguuga (SEQ ID NO: 4862), UCAguacgug (SEQ ID NO: 4863), AUGgucucuc (SEQ ID NO: 4864), UGAguuagua (SEQ ID NO: 4865), UGAguaaagu (SEQ ID NO: 4866), GAGgugaccg (SEQ ID NO: 4867), GAGguauauc (SEQ ID NO: 4868), CAGgugccau (SEQ ID NO: 4869), AGAgugguga (SEQ ID NO: 4870), GUUguaagaa (SEQ ID NO: 4871), AGAguaaaua (SEQ ID NO: 4872), AGGgugaagg (SEQ ID NO: 4873), CUGguagauu (SEQ ID NO: 4874), GAGguucagg (SEQ ID NO: 4875), AGGgucuuca (SEQ ID NO: 4876), CUGguaaccu (SEQ ID NO: 4877), ACAguacuga (SEQ ID NO: 4878), AGAguggguc (SEQ ID NO: 4879), AUGguaugag (SEQ ID NO: 4880), AAGguuauau (SEQ ID NO: 4881), AGAguauagu (SEQ ID NO: 4882), AAAguaugaa (SEQ ID NO: 4883), UAGguggcua (SEQ ID NO: 4884), ACCguauggg (SEQ ID NO: 4885), AAAguauaau (SEQ ID NO: 4886), UUUguauggc (SEQ ID NO: 4887), GGGgucgcgu (SEQ ID NO: 4888), GUGgugguuu (SEQ ID NO: 4889), CAGguuugac (SEQ ID NO: 4890), GGAguaggcg (SEQ ID NO: 4891), GAGguacccu (SEQ ID NO: 4892), AUGgugugca (SEQ ID NO: 4893), GUGguuggug (SEQ ID NO: 4894), AAAguaugcu (SEQ ID NO: 4895), UAAguuacau (SEQ ID NO: 4896), ACAguaugag (SEQ ID NO: 4897), GGAguauguu (SEQ ID NO: 4898), UUUgugagaa (SEQ ID NO: 4899), AAUgugcguu (SEQ ID NO: 4900), CAGguagagu (SEQ ID NO: 4901), AUGguguuaa (SEQ ID NO: 4902), CAUgugeguc (SEQ ID NO: 4903), AUAguuggau (SEQ ID NO: 4904), GAGguacgua (SEQ ID NO: 4905), GUUgugagaa (SEQ ID NO: 4906), CAAguacauc (SEQ ID NO: 4907), GAGguaguuu (SEQ ID NO: 4908), ACUguacaga (SEQ ID NO: 4909), CCGguuguga (SEQ ID NO: 4910), UGGgucagug (SEQ ID NO: 4911), GUAguaagaa (SEQ ID NO: 4912), GACguacuuu (SEQ ID NO: 4913), AGAgucaguc (SEQ ID NO: 4914), UAGguuaguu (SEQ ID NO: 4915), AGGgcagcag (SEQ ID NO: 4916), AAGguccuac (SEQ ID NO: 4917), AAUguaauug (SEQ ID NO: 4918), CAGgugcggg (SEQ ID NO: 4919), CUGguaaugg (SEQ ID NO: 4920), CAAguagccc (SEQ ID NO: 4921), GAAgucaguu (SEQ ID NO: 4922), ACAguaauug (SEQ ID NO: 4923), UUAguuagua (SEQ ID NO: 4924), CCUguauuuu (SEQ ID NO: 4925), AUCguaagaa (SEQ ID NO: 4926), CCAgugagca (SEQ ID NO: 4927), GAAguaaggc (SEQ ID NO: 4928), UGAgugggua (SEQ ID NO: 4929), UCAgugguag (SEQ ID NO: 4930), UCUguacagg (SEQ ID NO: 4931), CGAgugagug (SEQ ID NO: 4932), UCCguaugug (SEQ ID NO: 4933), CAUgccguuu (SEQ ID NO: 4934), AAAgugacuu (SEQ ID NO: 4935), AGAguaggca (SEQ ID NO: 4936), GAAguaagag (SEQ ID NO: 4937), CAGgcagguu (SEQ ID NO: 4938), UUGguagagc (SEQ ID NO: 4939), AAGguggaaa (SEQ ID NO: 4940), GAGgcagguc (SEQ ID NO: 4941), AUGguacgac (SEQ ID NO: 4942), AGGguaggaa (SEQ ID NO: 4943), AGGguaggua (SEQ ID NO: 4944), UUGguaaggu (SEQ ID NO: 4945), AUGguacaga (SEQ ID NO: 4946), CAGguagagc (SEQ ID NO: 4947), UAGguaaggu (SEQ ID NO: 4948), GGGguuagag (SEQ ID NO: 4949), AAGguaucaa (SEQ ID NO: 4950), GAGguagccc (SEQ ID NO: 4951), CAGgugccuc (SEQ ID NO: 4952), GCAguaagag (SEQ ID NO: 4953), ACGguagagu (SEQ ID NO: 4954), UGGguaaugg (SEQ ID NO: 4955), CUGgucaguu (SEQ ID NO: 4956), GUGguacauu (SEQ ID NO: 4957), AAAguagguu (SEQ ID NO: 4958), AAGgccaaga (SEQ ID NO: 4959), CGGgugggca (SEQ ID NO: 4960), ACGguccggg (SEQ ID NO: 4961), CGAguaugag (SEQ ID NO: 4962), CUGguaugcc (SEQ ID NO: 4963), GAGguggaug (SEQ ID NO: 4964), CAGgccuuuc (SEQ ID NO: 4965), AAAguacauc (SEQ ID NO: 4966), AAAguaauca (SEQ ID NO: 4967), GAGguaacug (SEQ ID NO: 4968), CUGguaaaga (SEQ ID NO: 4969), CGUguaagca (SEQ ID NO: 4970), UGGgcaagua (SEQ ID NO: 4971), GCGguggcga (SEQ ID NO: 4972), GAGguggccg (SEQ ID NO: 4973), AUUgcaugca (SEQ ID NO: 4974), ACGgugacug (SEQ ID NO: 4975), CAGgucagau (SEQ ID NO: 4976), AGAguaacuc (SEQ ID NO: 4977), UGAguaacag (SEQ ID NO: 4978), AAGguacccg (SEQ ID NO: 4979), AGGguaggcu (SEQ ID NO: 4980), GGGgcaggac (SEQ ID NO: 4981), CCUguaagug (SEQ ID NO: 4982), AUUguaagug (SEQ ID NO: 4983), ACUguacgag (SEQ ID NO: 4984), GUAguagugu (SEQ ID NO: 4985), AGAguaugag (SEQ ID NO: 4986), UCAguguggg (SEQ ID NO: 4987), UGGguauaua (SEQ ID NO: 4988), UAGguagcua (SEQ ID NO: 4989), GGGguaaaga (SEQ ID NO: 4990), AGGguuacuu (SEQ ID NO: 4991), CAUguaaaug (SEQ ID NO: 4992), GGAguaguaa (SEQ ID NO: 4993), CAGgucaauc (SEQ ID NO: 4994), CGGguuagug (SEQ ID NO: 4995), UAGguacaug (SEQ ID NO: 4996), UAGguuaaga (SEQ ID NO: 4997), UGGguaccuu (SEQ ID NO: 4998), CGGguggaca (SEQ ID NO: 4999), CAGgucuuac (SEQ ID NO: 5000), AAGguggagc (SEQ ID NO: 5001), AUGguaacca (SEQ ID NO: 5002), UCGguaaguu (SEQ ID NO: 5003), UAUguacaaa (SEQ ID NO: 5004), AAUguagauu (SEQ ID NO: 5005), GUAgcuagua (SEQ ID NO: 5006), AAGguauugg (SEQ ID NO: 5007), GAGgucuuug (SEQ ID NO: 5008), GAAguucagg (SEQ ID NO: 5009), UGGguaucac (SEQ ID NO: 5010), AGAguacugg (SEQ ID NO: 5011), CAGguuaaug (SEQ ID NO: 5012), AGGguacgug (SEQ ID NO: 5013), AGGgcacagg (SEQ ID NO: 5014), CUGguuaguu (SEQ ID NO: 5015), UUGguacgag (SEQ ID NO: 5016), ACGgugauca (SEQ ID NO: 5017), CCUgugagag (SEQ ID NO: 5018), GAGgugaagu (SEQ ID NO: 5019), AAGguacauc (SEQ ID NO: 5020), UCUguaugug (SEQ ID NO: 5021), UUGguggaag (SEQ ID NO: 5022), UGGgcagguu (SEQ ID NO: 5023), GAAguggagc (SEQ ID NO: 5024), ACAguaagac (SEQ ID NO: 5025), CGGguaccaa (SEQ ID NO: 5026), CAAguacguc (SEQ ID NO: 5027), AGAgugaggg (SEQ ID NO: 5028), CGGguaagaa (SEQ ID NO: 5029), AAUguaggug (SEQ ID NO: 5030), AUCgugugcu (SEQ ID NO: 5031), UAGgucaugg (SEQ ID NO: 5032), CAGguuuuga (SEQ ID NO: 5033), AAGgcaugca (SEQ ID NO: 5034), GAGgugcugc (SEQ ID NO: 5035), AAGguuaaua (SEQ ID NO: 5036), CAGguucauc (SEQ ID NO: 5037), GCGguaggug (SEQ ID NO: 5038), GACgugagua (SEQ ID NO: 5039), CAGgucuacu (SEQ ID NO: 5040), UUGguaugag (SEQ ID NO: 5041), AGCgugggca (SEQ ID NO: 5042), AUGguaaggu (SEQ ID NO: 5043), AUGguaccuc (SEQ ID NO: 5044), UUGguauggu (SEQ ID NO: 5045), UAUguaugaa (SEQ ID NO: 5046), UGGguauggg (SEQ ID NO: 5047), GAUguaaaua (SEQ ID NO: 5048), CCGguaaguu (SEQ ID NO: 5049), GAGgucugaa (SEQ ID NO: 5050), GAGgugcgag (SEQ ID NO: 5051), CUGgucagcc (SEQ ID NO: 5052), CAGguuuugu (SEQ ID NO: 5053), CGGguggugu (SEQ ID NO: 5054), UAAguuagua (SEQ ID NO: 5055), UUUgugugug (SEQ ID NO: 5056), CAGguuaacc (SEQ ID NO: 5057), UUGguacuuu (SEQ ID NO: 5058), GCUguaaggc (SEQ ID NO: 5059), AGGguggcug (SEQ ID NO: 5060), GAUguaaaaa (SEQ ID NO: 5061), AAGgucaaaa (SEQ ID NO: 5062), CAGguagcgc (SEQ ID NO: 5063), CAGguuuggc (SEQ ID NO: 5064), GAGgugguuu (SEQ ID NO: 5065), CGGguaaaua (SEQ ID NO: 5066), CUGguucggu (SEQ ID NO: 5067), GGAgugagcc (SEQ ID NO: 5068), AAGgugcgcg (SEQ ID NO: 5069), GAAguacauc (SEQ ID NO: 5070), AGUgucugua (SEQ ID NO: 5071), CCCgugagcu (SEQ ID NO: 5072), GAGguucaca (SEQ ID NO: 5073), CUAgugggua (SEQ ID NO: 5074), GAGguaacua (SEQ ID NO: 5075), UCGguauguc (SEQ ID NO: 5076), UAAguauuug (SEQ ID NO: 5077), CAGguaagcg (SEQ ID NO: 5078), GAGgugguaa (SEQ ID NO: 5079), CGAguaagag (SEQ ID NO: 5080), CCGguaagcu (SEQ ID NO: 5081), GAGgucuugu (SEQ ID NO: 5082), AAGguggguc (SEQ ID NO: 5083), CACguaagug (SEQ ID NO: 5084), AGUguaauga (SEQ ID NO: 5085), AAAgugugua (SEQ ID NO: 5086), GGAgugccaa (SEQ ID NO: 5087), CACgugaguu (SEQ ID NO: 5088), AAGguuggau (SEQ ID NO: 5089), UAUguaaaua (SEQ ID NO: 5090), CUGguaggaa (SEQ ID NO: 5091), UAUguaaacu (SEQ ID NO: 5092), AAUguauuuu (SEQ ID NO: 5093), CUGgcaagug (SEQ ID NO: 5094), UGUgugguau (SEQ ID NO: 5095), UAUguauguu (SEQ ID NO: 5096), UUGgugacuc (SEQ ID NO: 5097), GGAguaaggu (SEQ ID NO: 5098), AAGguagaug (SEQ ID NO: 5099), UGGguagggu (SEQ ID NO: 5100), AAUguaauuc (SEQ ID NO: 5101), GUGguauggc (SEQ ID NO: 5102), GGAguggguu (SEQ ID NO: 5103), AGGguaccac (SEQ ID NO: 5104), UAGgugacag (SEQ ID NO: 5105), ACAguaggca (SEQ ID NO: 5106), AUGguuugaa (SEQ ID NO: 5107), GCAguaacua (SEQ ID NO: 5108), CCGguaggua (SEQ ID NO: 5109), AGAguaggcc (SEQ ID NO: 5110), AAGguugaca (SEQ ID NO: 5111), CUGgugugua (SEQ ID NO: 5112), GAAgucuguc (SEQ ID NO: 5113), UGGgcucgga (SEQ ID NO: 5114), CAGguagccu (SEQ ID NO: 5115), AGAguaggua (SEQ ID NO: 5116), UAAguauguc (SEQ ID NO: 5117), CUGguauauc (SEQ ID NO: 5118), GAGguguguu (SEQ ID NO: 5119), AUGgugcaug (SEQ ID NO: 5120), AAGguacgcc (SEQ ID NO: 5121), UGAguaacua (SEQ ID NO: 5122), GAGgugacag (SEQ ID NO: 5123), GUUguccugu (SEQ ID NO: 5124), UUGgugucuu (SEQ ID NO: 5125), AAUgugaagg (SEQ ID NO: 5126), UUGguggaua (SEQ ID NO: 5127), UAGguguguu (SEQ ID NO: 5128), CUGgcaaguu (SEQ ID NO: 5129), GCAguaagau (SEQ ID NO: 5130), GCGguggaaa (SEQ ID NO: 5131), UGCguccagc (SEQ ID NO: 5132), AAAguggagu (SEQ ID NO: 5133), CGUgugagcc (SEQ ID NO: 5134), AGAguacugu (SEQ ID NO: 5135), CAGguauagc (SEQ ID NO: 5136), UACguaagga (SEQ ID NO: 5137), AAGgucuuua (SEQ ID NO: 5138), AAGguggucu (SEQ ID NO: 5139), GGGguaaauu (SEQ ID NO: 5140), UCAgugagga (SEQ ID NO: 5141), AGAguacguu (SEQ ID NO: 5142), GAGgucguca (SEQ ID NO: 5143), UAGguuugau (SEQ ID NO: 5144), CAUguaaacc (SEQ ID NO: 5145), AAGguggcac (SEQ ID NO: 5146), CAGguagaug (SEQ ID NO: 5147), AACguaaaag (SEQ ID NO: 5148), UAGgucucug (SEQ ID NO: 5149), AUAguaggug (SEQ ID NO: 5150), UAGgcaagag (SEQ ID NO: 5151), UAGgcacggc (SEQ ID NO: 5152), AAGgucuuca (SEQ ID NO: 5153), CCAguaugcu (SEQ ID NO: 5154), CAAgugaguu (SEQ ID NO: 5155), CAGgucucaa (SEQ ID NO: 5156), CAGguuacau (SEQ ID NO: 5157), GGAgugagca (SEQ ID NO: 5158), AGAguacgca (SEQ ID NO: 5159), CUGguguugg (SEQ ID NO: 5160), AAGguacuca (SEQ ID NO: 5161), CUAguaaggg (SEQ ID NO: 5162), AGAguaaaag (SEQ ID NO: 5163), AAGguaacga (SEQ ID NO: 5164), CUGguccccg (SEQ ID NO: 5165), UAAguauggg (SEQ ID NO: 5166), GAGgucgagc (SEQ ID NO: 5167), UUGguauaua (SEQ ID NO: 5168), AAAgucaagg (SEQ ID NO: 5169), AAGgucuagg (SEQ ID NO: 5170), CGAguagguc (SEQ ID NO: 5171), AGGguucguu (SEQ ID NO: 5172), GAGgcaggcc (SEQ ID NO: 5173), CUAguauuac (SEQ ID NO: 5174), ACGguaugug (SEQ ID NO: 5175), UAGgugguuc (SEQ ID NO: 5176), AGAguauaac (SEQ ID NO: 5177), UUGgugcguc (SEQ ID NO: 5178), ACCguuaucu (SEQ ID NO: 5179), CCAgugauga (SEQ ID NO: 5180), GAAguaugca (SEQ ID NO: 5181), GAAguauggc (SEQ ID NO: 5182), CCGguaggac (SEQ ID NO: 5183), AAUguaagca (SEQ ID NO: 5184), AGAguaauug (SEQ ID NO: 5185), AGGguugguu (SEQ ID NO: 5186), GUGguaggag (SEQ ID NO: 5187), AAGgcaguuu (SEQ ID NO: 5188), CAAguaagcc (SEQ ID NO: 5189), CUGgcaagua (SEQ ID NO: 5190), CAGgcaugau (SEQ ID NO: 5191), AGGguaauug (SEQ ID NO: 5192), GGGguaaccu (SEQ ID NO: 5193), AAAguaacua (SEQ ID NO: 5194), UAGgucugcc (SEQ ID NO: 5195), ACGguaugaa (SEQ ID NO: 5196), AGUguauggg (SEQ ID NO: 5197), UGGguuggca (SEQ ID NO: 5198), UAGguaaacu (SEQ ID NO: 5199), AGAgugggua (SEQ ID NO: 5200), AGAguauuug (SEQ ID NO: 5201), AGUguaggaa (SEQ ID NO: 5202), CUUguacgua (SEQ ID NO: 5203), GAUgugagau (SEQ ID NO: 5204), CAGgcagcca (SEQ ID NO: 5205), AAGgucacug (SEQ ID NO: 5206), AAGgucugac (SEQ ID NO: 5207), UAGguuccuu (SEQ ID NO: 5208), CUGgugcuuu (SEQ ID NO: 5209), UGAguuggug (SEQ ID NO: 5210), UUGgugggau (SEQ ID NO: 5211), UGAguagggu (SEQ ID NO: 5212), UCGgugaggu (SEQ ID NO: 5213), AAAguaaaga (SEQ ID NO: 5214), AAGgcaaguc (SEQ ID NO: 5215), CGGguaaagc (SEQ ID NO: 5216), AAAguuaguu (SEQ ID NO: 5217), UUAguaagca (SEQ ID NO: 5218), GAGgucacau (SEQ ID NO: 5219), UAAgugguau (SEQ ID NO: 5220), UAGgugcuuu (SEQ ID NO: 5221), GGAguaggca (SEQ ID NO: 5222), UGAguaagga (SEQ ID NO: 5223), CAGguggagc (SEQ ID NO: 5224), GAUguagaag (SEQ ID NO: 5225), AAUgccugcc (SEQ ID NO: 5226), AUGguaaggc (SEQ ID NO: 5227), UGGguaauau (SEQ ID NO: 5228), CUGguaccuc (SEQ ID NO: 5229), CACgugagcc (SEQ ID NO: 5230), UGAguuugug (SEQ ID NO: 5231), CCGguagugu (SEQ ID NO: 5232), AAAgugacaa (SEQ ID NO: 5233), GAAguggguu (SEQ ID NO: 5234), CAGgugcagc (SEQ ID NO: 5235), GAGgugggcc (SEQ ID NO: 5236), UAUgugcguc (SEQ ID NO: 5237), GGGguacugg (SEQ ID NO: 5238), CUGguagguu (SEQ ID NO: 5239), UUGgcauguu (SEQ ID NO: 5240), AAUguaauac (SEQ ID NO: 5241), UAGgccggug (SEQ ID NO: 5242), AGAgucagua (SEQ ID NO: 5243), UAAguaaauc (SEQ ID NO: 5244), CAGguuccuc (SEQ ID NO: 5245), UAGguacgau (SEQ ID NO: 5246), AGAguuagug (SEQ ID NO: 5247), GCAguaagug (SEQ ID NO: 5248), AGGgugguag (SEQ ID NO: 5249), GGAguaaugu (SEQ ID NO: 5250), GAUguaaguc (SEQ ID NO: 5251), CCAguuucgu (SEQ ID NO: 5252), AAGguucggg (SEQ ID NO: 5253), AUGguggagu (SEQ ID NO: 5254), AAGguaccgg (SEQ ID NO: 5255), GAAgugcgaa (SEQ ID NO: 5256), UGGgucaguu (SEQ ID NO: 5257), AAGguguaga (SEQ ID NO: 5258), UGGguaggcc (SEQ ID NO: 5259), CCAgugaguc (SEQ ID NO: 5260), AAGgucacuu (SEQ ID NO: 5261), AGCgugaggc (SEQ ID NO: 5262), UCCgugguaa (SEQ ID NO: 5263), AGAguacuua (SEQ ID NO: 5264), GGGgucagau (SEQ ID NO: 5265), AAGguggacc (SEQ ID NO: 5266), AGAgugagcg (SEQ ID NO: 5267), AGAgucagau (SEQ ID NO: 5268), UAAguauuac (SEQ ID NO: 5269), AGAguauuuc (SEQ ID NO: 5270), AGAguucagc (SEQ ID NO: 5271), AUGgugaagu (SEQ ID NO: 5272), UAGgugaucc (SEQ ID NO: 5273), GGAguaagau (SEQ ID NO: 5274), UAGguaccaa (SEQ ID NO: 5275), AGAguugguc (SEQ ID NO: 5276), GAAgugagac (SEQ ID NO: 5277), AUCguagguu (SEQ ID NO: 5278), GAGguacgcu (SEQ ID NO: 5279), ACGguaaggg (SEQ ID NO: 5280), CAGgcauguc (SEQ ID NO: 5281), UUAguaagau (SEQ ID NO: 5282), UGAguagguu (SEQ ID NO: 5283), AGGguacgaa (SEQ ID NO: 5284), ACGguauguu (SEQ ID NO: 5285), AGGguacugu (SEQ ID NO: 5286), UUGguaugga (SEQ ID NO: 5287), UAAguaacug (SEQ ID NO: 5288), GCGgucagcc (SEQ ID NO: 5289), UUUgugaguc (SEQ ID NO: 5290), GUGgucagug (SEQ ID NO: 5291), CUGgucugua (SEQ ID NO: 5292), GAGguucuua (SEQ ID NO: 5293), AUGguacuga (SEQ ID NO: 5294), AAUgugcuuu (SEQ ID NO: 5295), AGGguggcgu (SEQ ID NO: 5296), CCGgcaggaa (SEQ ID NO: 5297), CAUguggguc (SEQ ID NO: 5298), UUGguuuguu (SEQ ID NO: 5299), CAGguucugu (SEQ ID NO: 5300), ACGguaagcg (SEQ ID NO: 5301), CUGgucagua (SEQ ID NO: 5302), UCAguaggcu (SEQ ID NO: 5303), UGAguaggac (SEQ ID NO: 5304), CAGguuuuaa (SEQ ID NO: 5305), GAGguguccc (SEQ ID NO: 5306), AGGguggguu (SEQ ID NO: 5307), GUGgugagac (SEQ ID NO: 5308), CACguaggga (SEQ ID NO: 5309), GUGguauuuu (SEQ ID NO: 5310), GAGauauccu (SEQ ID NO: 5311), AAGgugaaca (SEQ ID NO: 5312), UAAguagggc (SEQ ID NO: 5313), CUGgugcggg (SEQ ID NO: 5314), CUGgucaaua (SEQ ID NO: 5315), AGAguaaaaa (SEQ ID NO: 5316), AAGgugcagu (SEQ ID NO: 5317), CGGguaagca (SEQ ID NO: 5318), AAAgugagcc (SEQ ID NO: 5319), AUGguaauca (SEQ ID NO: 5320), GCAguacgug (SEQ ID NO: 5321), AUGguacaug (SEQ ID NO: 5322), AAGguuaaga (SEQ ID NO: 5323), CGGguaaaug (SEQ ID NO: 5324), GAGguucgca (SEQ ID NO: 5325), GAGgcucugg (SEQ ID NO: 5326), AUGgugggac (SEQ ID NO: 5327), AACgugguag (SEQ ID NO: 5328), AAGgugauag (SEQ ID NO: 5329), GGGguuugca (SEQ ID NO: 5330), CAUguaaggg (SEQ ID NO: 5331), UCAguugagu (SEQ ID NO: 5332), AAAgugcggc (SEQ ID NO: 5333), AGAgugagcc (SEQ ID NO: 5334), AUGgcaagaa (SEQ ID NO: 5335), ACAguaaggu (SEQ ID NO: 5336), AAGgucucua (SEQ ID NO: 5337), GUGguaaaaa (SEQ ID NO: 5338), AAAguaggug (SEQ ID NO: 5339), UAGgugcacu (SEQ ID NO: 5340), GUCgugguau (SEQ ID NO: 5341), CAGguauagg (SEQ ID NO: 5342), UGAgugagag (SEQ ID NO: 5343), ACUgugagcc (SEQ ID NO: 5344), AUCguuaguu (SEQ ID NO: 5345), UUUguaccaa (SEQ ID NO: 5346), UGGgugagau (SEQ ID NO: 5347), AGAgugagaa (SEQ ID NO: 5348), AGAguagggg (SEQ ID NO: 5349), AGGgcaagua (SEQ ID NO: 5350), CGGgucagua (SEQ ID NO: 5351), UUGguaugcc (SEQ ID NO: 5352), CGGguuagau (SEQ ID NO: 5353), GGGgugaagu (SEQ ID NO: 5354), CCCgugugaa (SEQ ID NO: 5355), GCAguuugga (SEQ ID NO: 5356), UGCguaagac (SEQ ID NO: 5357), AGAgucugua (SEQ ID NO: 5358), CACgugagca (SEQ ID NO: 5359), AGGguaaaag (SEQ ID NO: 5360), CAGgcugggu (SEQ ID NO: 5361), GAAgucuuca (SEQ ID NO: 5362), AAGgcaaaaa (SEQ ID NO: 5363), GUAguaaaua (SEQ ID NO: 5364), CUAgugagag (SEQ ID NO: 5365), GAAguuucug (SEQ ID NO: 5366), CCUguacgua (SEQ ID NO: 5367), GAGgugcgcg (SEQ ID NO: 5368), AAGguguaaa (SEQ ID NO: 5369), CCAguauguu (SEQ ID NO: 5370), CCGgucagcu (SEQ ID NO: 5371), AUGguuccug (SEQ ID NO: 5372), CAAguuaaau (SEQ ID NO: 5373), AGAguaggcu (SEQ ID NO: 5374), AUGgugggca (SEQ ID NO: 5375), GGAguaagac (SEQ ID NO: 5376), AGGgucacga (SEQ ID NO: 5377), UAGgugauau (SEQ ID NO: 5378), GAAguaaguc (SEQ ID NO: 5379), CGGguaagau (SEQ ID NO: 5380), CAAguagcua (SEQ ID NO: 5381), UGAguaaaau (SEQ ID NO: 5382), GUCguacgug (SEQ ID NO: 5383), AUGguacgua (SEQ ID NO: 5384), CAGgucucgg (SEQ ID NO: 5385), GAGgcauguc (SEQ ID NO: 5386), AGAgugggau (SEQ ID NO: 5387), GUGguuagag (SEQ ID NO: 5388), UGGgugguga (SEQ ID NO: 5389), AAGguuaaac (SEQ ID NO: 5390), CUUguuagcu (SEQ ID NO: 5391), AAAguaggaa (SEQ ID NO: 5392), UAGguuguau (SEQ ID NO: 5393), AGGgugcgcc (SEQ ID NO: 5394), AAGgugggcu (SEQ ID NO: 5395), UAAguaucug (SEQ ID NO: 5396), AAGguaacgu (SEQ ID NO: 5397), AUGguggggc (SEQ ID NO: 5398), CAAguacacg (SEQ ID NO: 5399), GGCguaagug (SEQ ID NO: 5400), AUAguaggac (SEQ ID NO: 5401), AGAgugaggu (SEQ ID NO: 5402), UUUguaaaaa (SEQ ID NO: 5403), GAAguuugua (SEQ ID NO: 5404), CUAguaaucu (SEQ ID NO: 5405), AAGguuuuua (SEQ ID NO: 5406), GAGgugcguu (SEQ ID NO: 5407), UAGgcgagua (SEQ ID NO: 5408), ACCgugagua (SEQ ID NO: 5409), CAGgucccga (SEQ ID NO: 5410), AUGguacugg (SEQ ID NO: 5411), UGAguucagu (SEQ ID NO: 5412), AAUguguggu (SEQ ID NO: 5413), UCCguugguu (SEQ ID NO: 5414), CAGgucagag (SEQ ID NO: 5415), CAGgucccua (SEQ ID NO: 5416), UAGguagacu (SEQ ID NO: 5417), CAAguuaagg (SEQ ID NO: 5418), GAGgugugcg (SEQ ID NO: 5419), GAAgcugccc (SEQ ID NO: 5420), CGAguacgug (SEQ ID NO: 5421), CGGguaggua (SEQ ID NO: 5422), UUGguauuga (SEQ ID NO: 5423), AUUguaugau (SEQ ID NO: 5424), UUGguaugaa (SEQ ID NO: 5425), GAGgugguca (SEQ ID NO: 5426), GCUguaugaa (SEQ ID NO: 5427), CAGguguugc (SEQ ID NO: 5428), CAGguaaaac (SEQ ID NO: 5429), AUAguaaggu (SEQ ID NO: 5430), CUGguuagag (SEQ ID NO: 5431), AGCgugugag (SEQ ID NO: 5432), AAGguuaucu (SEQ ID NO: 5433), CACgugagua (SEQ ID NO: 5434), AGGgucagua (SEQ ID NO: 5435), GAGguauaau (SEQ ID NO: 5436), CAGguuauuu (SEQ ID NO: 5437), AGGguggacu (SEQ ID NO: 5438), AUUguaauuc (SEQ ID NO: 5439), UUUguggguu (SEQ ID NO: 5440), AUGguacgug (SEQ ID NO: 5441), AAGguguucc (SEQ ID NO: 5442), CAGgugacgc (SEQ ID NO: 5443), GAGguacuaa (SEQ ID NO: 5444), ACAguucagu (SEQ ID NO: 5445), GAGgucacgg (SEQ ID NO: 5446), CAAguaaggc (SEQ ID NO: 5447), AAGguuuggg (SEQ ID NO: 5448), AAAgugggcu (SEQ ID NO: 5449), GCGguucuug (SEQ ID NO: 5450), GAGguggagc (SEQ ID NO: 5451), UGAgucagug (SEQ ID NO: 5452), CAGgucaagg (SEQ ID NO: 5453), AGUguaagcu (SEQ ID NO: 5454), GAGgcagaaa (SEQ ID NO: 5455), AAGgucacac (SEQ ID NO: 5456), GAAguagguu (SEQ ID NO: 5457), GUCguaaguu (SEQ ID NO: 5458), AGAguaugca (SEQ ID NO: 5459), CCUgugcaaa (SEQ ID NO: 5460), ACGgugaaaa (SEQ ID NO: 5461), CAGguacgaa (SEQ ID NO: 5462), CAUgugagga (SEQ ID NO: 5463), AGCgugagua (SEQ ID NO: 5464), GGUguguagg (SEQ ID NO: 5465), AACgugagcu (SEQ ID NO: 5466), GAGgugaacu (SEQ ID NO: 5467), AGAguucagu (SEQ ID NO: 5468), AACgugugua (SEQ ID NO: 5469), CAGguugugg (SEQ ID NO: 5470), AAGguacuag (SEQ ID NO: 5471), UCAgugaaaa (SEQ ID NO: 5472), AAUgucuggu (SEQ ID NO: 5473), ACGguaaaau (SEQ ID NO: 5474), CUGguguaag (SEQ ID NO: 5475), GAGgugcgaa (SEQ ID NO: 5476), AGGguuucuc (SEQ ID NO: 5477), CAGguagccc (SEQ ID NO: 5478), AUUguauugg (SEQ ID NO: 5479), AUGguacuua (SEQ ID NO: 5480), GAGgcccgac (SEQ ID NO: 5481), UCGguaagac (SEQ ID NO: 5482), CGGgcuguag (SEQ ID NO: 5483), UAUgugugug (SEQ ID NO: 5484), UAGguagaaa (SEQ ID NO: 5485), GUGgucauua (SEQ ID NO: 5486), UAGgugaaag (SEQ ID NO: 5487), ACUguaauuc (SEQ ID NO: 5488), GCAguacagg (SEQ ID NO: 5489), UCGgugaguc (SEQ ID NO: 5490), UAUguaggga (SEQ ID NO: 5491), AUGguauguc (SEQ ID NO: 5492), GUGgugugug (SEQ ID NO: 5493), CUGgugaccu (SEQ ID NO: 5494), AAUgugaaua (SEQ ID NO: 5495), UAGgucucac (SEQ ID NO: 5496), GAGguuauug (SEQ ID NO: 5497), UGAguaggcu (SEQ ID NO: 5498), CGGgcacgua (SEQ ID NO: 5499), GCAguaaaua (SEQ ID NO: 5500), CCGgugagag (SEQ ID NO: 5501), UAAguugguc (SEQ ID NO: 5502), CCGgugagcc (SEQ ID NO: 5503), AAGguuguca (SEQ ID NO: 5504), CUGguauuau (SEQ ID NO: 5505), GGGguauggg (SEQ ID NO: 5506), AAAgucagua (SEQ ID NO: 5507), UUUguaugua (SEQ ID NO: 5508), UAAguacugc (SEQ ID NO: 5509), CAGguaccaa (SEQ ID NO: 5510), GAAguucaga (SEQ ID NO: 5511), AUGgugcggu (SEQ ID NO: 5512), GUGgugaggu (SEQ ID NO: 5513), UGAguaagcc (SEQ ID NO: 5514), UAUguaaggg (SEQ ID NO: 5515), GUGguggaaa (SEQ ID NO: 5516), GAGgugauug (SEQ ID NO: 5517), GGAguuugua (SEQ ID NO: 5518), AAGgucacga (SEQ ID NO: 5519), GUGguagagg (SEQ ID NO: 5520), UAAguauauc (SEQ ID NO: 5521), AAGgugucca (SEQ ID NO: 5522), UAUgugguau (SEQ ID NO: 5523), GAGguacaau (SEQ ID NO: 5524), AAGguggggg (SEQ ID NO: 5525), GGAguaggug (SEQ ID NO: 5526), and UAGgugacuu (SEQ ID NO: 5527).

In some embodiments, the splice site sequence (e.g., 5′ splice site sequence) comprises AGA. In some embodiments, the splice site sequence (e.g., 5′ splice site sequence) comprises AAA. In some embodiments, the splice site sequence (e.g., 5′ splice site sequence) comprises AAC. In some embodiments, the splice site sequence (e.g., 5′ splice site sequence) comprises AAU. In some embodiments, the splice site sequence (e.g., 5′ splice site sequence) comprises AAG. In some embodiments, the splice site sequence (e.g., 5′ splice site sequence) comprises ACA. In some embodiments, the splice site sequence (e.g., 5′ splice site sequence) comprises AUA. In some embodiments, the splice site sequence (e.g., 5′ splice site sequence) comprises AUU. In some embodiments, the splice site sequence (e.g., 5′ splice site sequence) comprises AUG. In some embodiments, the splice site sequence (e.g., 5′ splice site sequence) comprises AUC. In some embodiments, the splice site sequence (e.g., 5′ splice site sequence) comprises CAA. In some embodiments, the splice site sequence (e.g., 5′ splice site sequence) comprises CAU. In some embodiments, the splice site sequence (e.g., 5′ splice site sequence) comprises CAC. In some embodiments, the splice site sequence (e.g., 5′ splice site sequence) comprises CAG. In some embodiments, the splice site sequence (e.g., 5′ splice site sequence) comprises GAA. In some embodiments, the splice site sequence (e.g., 5′ splice site sequence) comprises GAC. In some embodiments, the splice site sequence (e.g., 5′ splice site sequence) comprises GAU. In some embodiments, the splice site sequence (e.g., 5′ splice site sequence) comprises GAG. In some embodiments, the splice site sequence (e.g., 5′ splice site sequence) comprises GGA. In some embodiments, the splice site sequence (e.g., 5′ splice site sequence) comprises GCA. In some embodiments, the splice site sequence (e.g., 5′ splice site sequence) comprises GGG. In some embodiments, the splice site sequence (e.g., 5′ splice site sequence) comprises GGC. In some embodiments, the splice site sequence (e.g., 5′ splice site sequence) comprises GUU. In some embodiments, the splice site sequence (e.g., 5′ splice site sequence) comprises GGU. In some embodiments, the splice site sequence (e.g., 5′ splice site sequence) comprises GUC. In some embodiments, the splice site sequence (e.g., 5′ splice site sequence) comprises GUA. In some embodiments, the splice site sequence (e.g., 5′ splice site sequence) comprises GUG. In some embodiments, the splice site sequence (e.g., 5′ splice site sequence) comprises UCU. In some embodiments, the splice site sequence (e.g., 5′ splice site sequence) comprises UCC. In some embodiments, the splice site sequence (e.g., 5′ splice site sequence) comprises UCA. In some embodiments, the splice site sequence (e.g., 5′ splice site sequence) comprises UCG. In some embodiments, the splice site sequence (e.g., 5′ splice site sequence) comprises UUU. In some embodiments, the splice site sequence (e.g., 5′ splice site sequence) comprises UUC. In some embodiments, the splice site sequence (e.g., 5′ splice site sequence) comprises UUA. In some embodiments, the splice site sequence (e.g., 5′ splice site sequence) comprises UUG. In some embodiments, the splice site sequence (e.g., 5′ splice site sequence) comprises UGU. In some embodiments, the splice site sequence (e.g., 5′ splice site sequence) comprises UAU. In some embodiments, the splice site sequence (e.g., 5′ splice site sequence) comprises GGA. In some embodiments, the splice site sequence (e.g., 5′ splice site sequence) comprises CUU. In some embodiments, the splice site sequence (e.g., 5′ splice site sequence) comprises CUC. In some embodiments, the splice site sequence (e.g., 5′ splice site sequence) comprises CUA. In some embodiments, the splice site sequence (e.g., 5′ splice site sequence) comprises CUG. In some embodiments, the splice site sequence (e.g., 5′ splice site sequence) comprises CCU. In some embodiments, the splice site sequence (e.g., 5′ splice site sequence) comprises CCC. In some embodiments, the splice site sequence (e.g., 5′ splice site sequence) comprises CCA. In some embodiments, the splice site sequence (e.g., 5′ splice site sequence) comprises CCG. In some embodiments, the splice site sequence (e.g., 5′ splice site sequence) comprises ACU. In some embodiments, the splice site sequence (e.g., 5′ splice site sequence) comprises ACC. In some embodiments, the splice site sequence (e.g., 5′ splice site sequence) comprises ACG. In some embodiments, the splice site sequence (e.g., 5′ splice site sequence) comprises AGC. In some embodiments, the splice site sequence (e.g., 5′ splice site sequence) comprises AGU. In some embodiments, the splice site sequence (e.g., 5′ splice site sequence) comprises AGG. In some embodiments, the splice site sequence (e.g., 5′ splice site sequence) comprises CGU. In some embodiments, the splice site sequence (e.g., 5′ splice site sequence) comprises UAC. In some embodiments, the splice site sequence (e.g., 5′ splice site sequence) comprises UAA. In some embodiments, the splice site sequence (e.g., 5′ splice site sequence) comprises UAG. In some embodiments, the splice site sequence (e.g., 5′ splice site sequence) comprises CGC. In some embodiments, the splice site sequence (e.g., 5′ splice site sequence) comprises CGA. In some embodiments, the splice site sequence (e.g., 5′ splice site sequence) comprises CGG. In some embodiments, the splice site sequence comprises AGAguaaggg (SEQ ID NO: 667). In some embodiments, the splice site sequence comprises UGAguaagca (SEQ ID NO: 2768).

In an embodiment, a gene sequence or splice site sequence provided herein is related to a proliferative disease, disorder, or condition (e.g., cancer, benign neoplasm, or inflammatory disease). In an embodiment, a gene sequence or splice site sequence provided herein is related to a non-proliferative disease, disorder, or condition. In an embodiment, a gene sequence or splice site sequence provided herein is related to a neurological disease or disorder; autoimmune disease or disorder; immunodeficiency disease or disorder; lysosomal storage disease or disorder; cardiovascular condition, disease or disorder; metabolic disease or disorder; respiratory condition, disease, or disorder; renal disease or disorder; or infectious disease in a subject. In an embodiment, a gene sequence or splice site sequence provided herein is related to a neurological disease or disorder (e.g., Huntington's disease). In an embodiment, a gene sequence or splice site sequence provided herein is related to an immunodeficiency disease or disorder. In an embodiment, a gene sequence or splice site sequence provided herein is related to a lysosomal storage disease or disorder. In an embodiment, a gene sequence or splice site sequence provided herein is related to a cardiovascular condition, disease or disorder. In an embodiment, a gene sequence or splice site sequence provided herein is related to a metabolic disease or disorder. In an embodiment, a gene sequence or splice site sequence provided herein is related to a respiratory condition, disease, or disorder. In an embodiment, a gene sequence or splice site sequence provided herein is related to a renal disease or disorder. In an embodiment, a gene sequence or splice site sequence provided herein is related to an infectious disease.

In an embodiment, a gene sequence or splice site sequence provided herein is related to a mental retardation disorder. In an embodiment, a gene sequence or splice site sequence provided herein is related to a mutation in the SETD5 gene. In an embodiment, a gene sequence or splice site sequence provided herein is related to an immunodeficiency disorder. In an embodiment, a gene sequence and splice site sequence provided herein is related to a mutation in the GATA2 gene. In an embodiment, a gene sequence or splice site sequence provided herein is related to a lysosomal storage disease.

In some embodiments, a compound of Formula (I) or (II) described herein interacts with (e.g., binds to) a splicing complex component (e.g., a nucleic acid (e.g., an RNA) or a protein). In some embodiments, the splicing complex component is selected from 9G8, Al hnRNP, A2 hnRNP, ASD-1, ASD-2b, ASF, BRR2, B1 hnRNP, C1 hnRNP, C2 hnRNP, CBP20, CBP80, CELF, F hnRNP, FBP11, Fox-1, Fox-2, G hnRNP, H hnRNP, hnRNP 1, hnRNP 3, hnRNP C, hnRNP G, hnRNP K, hnRNP M, hnRNP U, Hu, HUR, I hnRNP, K hnRNP, KH-type splicing regulatory protein (KSRP), L hnRNP, LUC7L, M hnRNP, mBBP, muscle-blind like (MBNL), NF45, NFAR, Nova-1, Nova-2, nPTB, P54/SFRS11, polypyrimidine tract binding protein (PTB), a PRP protein (e.g., PRP8, PRP6, PRP31, PRP4, PRP3, PRP28, PRP5, PRP2, PRP19), PRP19 complex proteins, RBM42, R hnRNP, RNPC1, SAD1, SAM68, SC35, SF, SF1/BBP, SF2, SF3A complex, SF3B complex, SFRS10, an Sm protein (such as B, D1, D2, D3, F, E, G), SNU17, SNU66, SNU114, an SR protein, SRm300, SRp20, SRp30c, SRP35C, SRP36, SRP38, SRp40, SRp55, SRp75, SRSF, STAR, GSG, SUP-12, TASR-1, TASR-2, TIA, TIAR, TRA2, TRA2a/b, U hnRNP, Ul snRNP, U11 snRNP, U12 snRNP, U1-70K, U1-A, U1-C, U2 snRNP, U2AF1-RS2, U2AF35, U2AF65, U4 snRNP, U5 snRNP, U6 snRNP, Urp, and YB1.

In some embodiments, the splicing complex component comprises RNA (e.g., snRNA). In some embodiments, a compound described herein binds to a splicing complex component comprising snRNA. The snRNA may be selected from, e.g., U1 snRNA, U2 snRNA, U4 snRNA, U5 snRNA, U6 snRNA, U11 snRNA, U12 snRNA, U4atac snRNA, and any combination thereof.

In some embodiments, the splicing complex component comprises a protein, e.g., a protein associated with an snRNA. In some embodiments, the protein comprises SC35, SRp55, SRp40, SRm300, SFRS10, TASR-1, TASR-2, SF2/ASF, 9G8, SRp75, SRp30c, SRp20 and P54/SFRS11. In some embodiments, the splicing complex component comprises a U2 snRNA auxiliary factor (e.g., U2AF65, U2AF35), Urp/U2AF1-RS2, SF1/BBP, CBP80, CBP 20, SF1 or PTB/hnRNPi. In some embodiments, the hnRNP protein comprises A1, A2/B1, L, M, K, U, F, H, G, R, I or C1/C2. Human genes encoding hnRNPs include HNRNPA0, HNRNPA1, HNRNPA1L1, HNRNPA1L2, HNRNPA3, HNRNPA2B1, HNRNPAB, HNRNPB1, HNRNPC, HNRNPCL1, HNRNPD, HNRPDL, HNRNPF, HNRNPH1, HNRNPH2, HNRNPH3, HNRNPK, HNRNPL, HNRPLL, HNRNPM, HNRNPR, HNRNPU, HNRNPUL1, HNRNPUL2, HNRNPUL3, and FMR1.

In one aspect, the compounds of Formula (I) or (II) and pharmaceutically acceptable salts, solvates, hydrates, tautomers, stereoisomers, and compositions thereof, may modulate (e.g., increase or decrease) a splicing event of a target nucleic acid sequence (e.g., DNA, RNA, or a pre-mRNA), for example, a nucleic acid encoding a gene described herein, or a nucleic acid encoding a protein described herein, or a nucleic acid comprising a splice site described herein. In an embodiment, the splicing event is an alternative splicing event.

In an embodiment, the compound of Formula (I) or (II) or a pharmaceutically acceptable salt, solvate, hydrate, tautomer, stereoisomer, and compositions thereof increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR. In an embodiment, the compound of Formula (I) or (II) or a pharmaceutically acceptable salt, solvate, hydrate, tautomer, stereoisomer, and compositions thereof decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by a known method in the art, e.g., qPCR.

In another aspect, the present disclosure features a method of forming a complex comprising a component of a spliceosome (e.g., a major spliceosome component or a minor spliceosome component), a nucleic acid (e.g., a DNA, RNA, e.g., a pre-mRNA), and a compound of Formula (I) or (II) or a pharmaceutically acceptable salt, solvate, hydrate, tautomer, stereoisomer, or composition thereof, comprising contacting the nucleic acid (e.g., a DNA, RNA, e.g., a pre-mRNA) with said compound of Formula (I) or (II). In an embodiment, the component of a spliceosome is selected from the U1, U2, U4, U5, U6, U11, U12, U4atac, U6atac small nuclear ribonucleoproteins (snRNPs), or a related accessory factor. In an embodiment, the component of a spliceosome is recruited to the nucleic acid in the presence of the compound of Formula (I) or (II) or a pharmaceutically acceptable salt, solvate, hydrate, tautomer, stereoisomer, or composition thereof.

In another aspect, the present disclosure features a method of altering the conformation of a nucleic acid (e.g., a DNA, RNA, e.g., a pre-mRNA) comprising contacting the nucleic acid with a compound of Formula (I) or (II) or a pharmaceutically acceptable salt, solvate, hydrate, tautomer, stereoisomer, or composition thereof. In an embodiment, the altering comprises forming a bulge or kink in the nucleic acid. In an embodiment, the altering comprises stabilizing a bulge or a kink in the nucleic acid. In an embodiment, the altering comprises reducing a bulge or a kink in the nucleic acid. In an embodiment, the nucleic acid comprises a splice site. In an embodiment, the compound of Formula (I) or (II) interacts with a nucleobase, ribose, or phosphate moiety of a nucleic acid (e.g., a DNA, RNA, e.g., pre-mRNA).

The present disclosure also provides methods for the treatment or prevention of a disease, disorder, or condition. In an embodiment, the disease, disorder or condition is related to (e.g., caused by) a splicing event, such as an unwanted, aberrant, or alternative splicing event. In an embodiment, the disease, disorder or condition comprises a proliferative disease (e.g., cancer, benign neoplasm, or inflammatory disease) or non-proliferative disease. In an embodiment, the disease, disorder, or condition comprises a neurological disease, autoimmune disorder, immunodeficiency disorder, cardiovascular condition, metabolic disorder, lysosomal storage disease, respiratory condition, renal disease, or infectious disease in a subject. In another embodiment, the disease, disorder, or condition comprises a haploinsufficiency disease, an autosomal recessive disease (e.g., with residual function), or a paralogue activation disorder. In another embodiment, the disease, disorder, or condition comprises an autosomal dominant disorder (e.g., with residual function). Such methods comprise the step of administering to the subject in need thereof an effective amount of a compound of Formula (I) or (II), or a pharmaceutically acceptable salt, solvate, hydrate, tautomer, stereoisomer thereof, or a pharmaceutical composition thereof. In certain embodiments, the methods described herein include administering to a subject an effective amount of a compound of Formula (I) or (II), or a pharmaceutically acceptable salt thereof, or a pharmaceutical composition thereof.

In certain embodiments, the subject being treated is a mammal. In certain embodiments, the subject is a human. In certain embodiments, the subject is a domesticated animal, such as a dog, cat, cow, pig, horse, sheep, or goat. In certain embodiments, the subject is a companion animal such as a dog or cat. In certain embodiments, the subject is a livestock animal such as a cow, pig, horse, sheep, or goat. In certain embodiments, the subject is a zoo animal. In another embodiment, the subject is a research animal such as a rodent, dog, or non-human primate. In certain embodiments, the subject is a non-human transgenic animal such as a transgenic mouse or transgenic pig.

A proliferative disease may also be associated with inhibition of apoptosis of a cell in a biological sample or subject. All types of biological samples described herein or known in the art are contemplated as being within the scope of the disclosure. The compounds of Formula (I) or (II) and pharmaceutically acceptable salts, solvates, hydrates, tautomers, stereoisomers, and compositions thereof, may induce apoptosis, and therefore, be useful in treating and/or preventing proliferative diseases.

In certain embodiments, the proliferative disease to be treated or prevented using the compounds of Formula (I) or (II) is cancer. As used herein, the term “cancer” refers to a malignant neoplasm (Stedman's Medical Dictionary, 25th ed.; Hensyl ed.; Williams & Wilkins: Philadelphia, 1990). All types of cancers disclosed herein or known in the art are contemplated as being within the scope of the disclosure. Exemplary cancers include, but are not limited to, acoustic neuroma; adenocarcinoma; adrenal gland cancer; anal cancer; angiosarcoma (e.g., lymphangiosarcoma, lymphangioendotheliosarcoma, hemangiosarcoma); appendix cancer; benign monoclonal gammopathy; biliary cancer (e.g., cholangiocarcinoma); bladder cancer; breast cancer (e.g., adenocarcinoma of the breast, papillary carcinoma of the breast, mammary cancer, medullary carcinoma of the breast); brain cancer (e.g., meningioma, glioblastomas, glioma (e.g., astrocytoma, oligodendroglioma), medulloblastoma); bronchus cancer; carcinoid tumor; cervical cancer (e.g., cervical adenocarcinoma); choriocarcinoma; chordoma; craniopharyngioma; colorectal cancer (e.g., colon cancer, rectal cancer, colorectal adenocarcinoma); connective tissue cancer; epithelial carcinoma; ependymoma; endotheliosarcoma (e.g., Kaposi's sarcoma, multiple idiopathic hemorrhagic sarcoma); endometrial cancer (e.g., uterine cancer, uterine sarcoma); esophageal cancer (e.g., adenocarcinoma of the esophagus, Barrett's adenocarcinoma); Ewing's sarcoma; eye cancer (e.g., intraocular melanoma, retinoblastoma); familiar hypereosinophilia; gall bladder cancer; gastric cancer (e.g., stomach adenocarcinoma); gastrointestinal stromal tumor (GIST); germ cell cancer; head and neck cancer (e.g., head and neck squamous cell carcinoma, oral cancer (e.g., oral squamous cell carcinoma), throat cancer (e.g., laryngeal cancer, pharyngeal cancer, nasopharyngeal cancer, oropharyngeal cancer), e.g., adenoid cystic carcinoma (ACC)); hematopoietic cancers (e.g., leukemia such as acute lymphocytic leukemia (ALL) (e.g., B-cell ALL, T-cell ALL), acute myelocytic leukemia (AML) (e.g., B-cell AML, T-cell AML), chronic myelocytic leukemia (CML) (e.g., B-cell CML, T-cell CML), and chronic lymphocytic leukemia (CLL) (e.g., B-cell CLL, T-cell CLL)); lymphoma such as Hodgkin lymphoma (HL) (e.g., B-cell HL, T-cell HL) and non-Hodgkin lymphoma (NHL) (e.g., B-cell NHL such as diffuse large cell lymphoma (DLCL) (e.g., diffuse large B-cell lymphoma), follicular lymphoma, chronic lymphocytic leukemia/small lymphocytic lymphoma (CLL/SLL), mantle cell lymphoma (MCL), marginal zone B-cell lymphomas (e.g., mucosa-associated lymphoid tissue (MALT) lymphomas, nodal marginal zone B-cell lymphoma, splenic marginal zone B-cell lymphoma), primary mediastinal B-cell lymphoma, Burkitt lymphoma, lymphoplasmacytic lymphoma (i.e., Waldenstrom's macroglobulinemia), hairy cell leukemia (HCL), immunoblastic large cell lymphoma, precursor B-lymphoblastic lymphoma and primary central nervous system (CNS) lymphoma; and T-cell NHL such as precursor T-lymphoblastic lymphoma/leukemia, peripheral T-cell lymphoma (PTCL) (e.g., cutaneous T-cell lymphoma (CTCL) (e.g., mycosis fungoides, Sezary syndrome), angioimmunoblastic T-cell lymphoma, extranodal natural killer T-cell lymphoma, enteropathy type T-cell lymphoma, subcutaneous panniculitis-like T-cell lymphoma, and anaplastic large cell lymphoma); a mixture of one or more leukemia/lymphoma as described above; and multiple myeloma (MM)), heavy chain disease (e.g., alpha chain disease, gamma chain disease, mu chain disease); hemangioblastoma; hypopharynx cancer; inflammatory myofibroblastic tumors; immunocytic amyloidosis; kidney cancer (e.g., nephroblastoma a.k.a. Wilms' tumor, renal cell carcinoma); liver cancer (e.g., hepatocellular cancer (HCC), malignant hepatoma); lung cancer (e.g., bronchogenic carcinoma, small cell lung cancer (SCLC), non-small cell lung cancer (NSCLC), adenocarcinoma of the lung); leiomyosarcoma (LMS); mastocytosis (e.g., systemic mastocytosis); muscle cancer; myelodysplastic syndrome (MDS); mesothelioma; myeloproliferative disorder (MPD) (e.g., polycythemia vera (PV), essential thrombocytosis (ET), agnogenic myeloid metaplasia (AMM) a.k.a. myelofibrosis (MF), chronic idiopathic myelofibrosis, chronic myelocytic leukemia (CML), chronic neutrophilic leukemia (CNL), hypereosinophilic syndrome (HES)); neuroblastoma; neurofibroma (e.g., neurofibromatosis (NF) type 1 or type 2, schwannomatosis); neuroendocrine cancer (e.g., gastroenteropancreatic neuroendocrine tumor (GEP-NET), carcinoid tumor); osteosarcoma (e.g., bone cancer); ovarian cancer (e.g., cystadenocarcinoma, ovarian embryonal carcinoma, ovarian adenocarcinoma); papillary adenocarcinoma; pancreatic cancer (e.g., pancreatic adenocarcinoma, intraductal papillary mucinous neoplasm (IPMN), Islet cell tumors); penile cancer (e.g., Paget's disease of the penis and scrotum); pinealoma; primitive neuroectodermal tumor (PNT); plasma cell neoplasia; paraneoplastic syndromes; intraepithelial neoplasms; prostate cancer (e.g., prostate adenocarcinoma); rectal cancer; rhabdomyosarcoma; salivary gland cancer; skin cancer (e.g., squamous cell carcinoma (SCC), keratoacanthoma (KA), melanoma, basal cell carcinoma (BCC)); small bowel cancer (e.g., appendix cancer); soft tissue sarcoma (e.g., malignant fibrous histiocytoma (MFH), liposarcoma, malignant peripheral nerve sheath tumor (MPNST), chondrosarcoma, fibrosarcoma, myxosarcoma); sebaceous gland carcinoma; small intestine cancer; sweat gland carcinoma; synovioma; testicular cancer (e.g., seminoma, testicular embryonal carcinoma); thyroid cancer (e.g., papillary carcinoma of the thyroid, papillary thyroid carcinoma (PTC), medullary thyroid cancer); urethral cancer; vaginal cancer; and vulvar cancer (e.g., Paget's disease of the vulva).

In some embodiments, the cancer is selected from adenoid cystic carcinoma (ACC), acute myelocytic leukemia (AML) (e.g., B-cell AML, T-cell AML), chronic myelocytic leukemia (CML) (e.g., B-cell CML, T-cell CML), non-Hodgkin lymphoma (NHL), Burkitt lymphoma, colorectal cancer (e.g., colon cancer, rectal cancer, colorectal adenocarcinoma), prostate cancer (e.g., prostate adenocarcinoma), ovarian cancer (e.g., cystadenocarcinoma, ovarian embryonal carcinoma, ovarian adenocarcinoma), and myelodysplastic syndrome (MDS).

In some embodiments, the proliferative disease is associated with a benign neoplasm. For example, a benign neoplasm may include adenoma, fibroma, hemangioma, tuberous sclerosis, and lipoma. All types of benign neoplasms disclosed herein or known in the art are contemplated as being within the scope of the disclosure.

In some embodiments, the proliferative disease is associated with angiogenesis. All types of angiogenesis disclosed herein or known in the art are contemplated as being within the scope of the disclosure.

In some embodiments, the compound of Formula (I) or (II) or a pharmaceutically acceptable salt thereof, or compositions comprising such compound or pharmaceutically acceptable salt thereof, is used to prevent or treat a non-proliferative disease. Exemplary non-proliferative diseases include a neurological disease, autoimmune disorder, immunodeficiency disorder, lysosomal storage disease, cardiovascular condition, metabolic disorder, respiratory condition, inflammatory disease, renal disease, or infectious disease.

In certain embodiments, the non-proliferative disease is a neurological disease. In certain embodiments, the compound of Formula (I) or (II), or a pharmaceutically acceptable salt thereof, or compositions comprising such compound or pharmaceutically acceptable salt thereof, is used to prevent or treat a neurological disease, disorder, or condition. A neurological disease, disorder, or condition may include a neurodegenerative disease, a psychiatric condition, or a musculoskeletal disease. A neurological disease may further include a repeat expansion disease, e.g., which may be characterized by the expansion of a nucleic acid sequence in the genome. For example, a repeat expansion disease includes myotonic dystrophy, amyotrophic lateral sclerosis, Huntington's disease, a trinucleotide repeat disease, or a polyglutamine disorder (e.g., ataxia, fragile X syndrome). In some embodiments, the neurological disease comprises a repeat expansion disease, e.g., Huntington's disease. Additional neurological diseases, disorders, and conditions include Alzheimer's disease, Huntington's chorea, a prion disease (e.g., Creutzfeld-Jacob disease, bovine spongiform encephalopathy, Kuru, or scrapie), a mental retardation disorder (e.g., a disorder caused by a SETD5 gene mutation, e.g., intellectual disability-facial dysmorphism syndrome, autism spectrum disorder), Lewy Body disease, diffuse Lewy body disease (DLBD), dementia, progressive supranuclear palsy (PSP), progressive bulbar palsy (PBP), psuedobulbar palsy, spinal and bulbar muscular atrophy (SBMA), primary lateral sclerosis, Pick's disease, primary progressive aphasia, corticobasal dementia, Parkinson's disease, Down's syndrome, multiple system atrophy, spinal muscular atrophy (SMA), progressive spinobulbar muscular atrophy (e.g., Kennedy disease), post-polio syndrome (PPS), spinocerebellar ataxia, pantothenate kinase-associated neurodegeneration (PANK), spinal degenerative disease/motor neuron degenerative diseases, upper motor neuron disorder, lower motor neuron disorder, Hallervorden-Spatz syndrome, cerebral infarction, cerebral trauma, chronic traumatic encephalopathy, transient ischemic attack, Lytigo-bodig (amyotrophic lateral sclerosis-parkinsonism dementia), Guam-Parkinsonism dementia, hippocampal sclerosis, corticobasal degeneration, Alexander disease, Apler's disease, Krabbe's disease, neuroborreliosis, neurosyphilis, Sandhoff disease, Tay-Sachs disease, Schilder's disease, Batten disease, Cockayne syndrome, Kearns-Sayre syndrome, Gerstmann-Straussler-Scheinker syndrome and other transmissible spongiform encephalopathies, hereditary spastic paraparesis, Leigh's syndrome, a demyelinating diseases, neuronal ceroid lipofuscinoses, epilepsy, tremors, depression, mania, anxiety and anxiety disorders, sleep disorders (e.g., narcolepsy, fatal familial insomnia), acute brain injuries (e.g., stroke, head injury), autism, Machado-Joseph disease, or a combination thereof. In some embodiments, the neurological disease comprises Friedrich's ataxia or Sturge Weber syndrome. In some embodiments, the neurological disease comprises Huntington's disease. In some embodiments, the neurological disease comprises spinal muscular atrophy. All types of neurological diseases disclosed herein or known in the art are contemplated as being within the scope of the disclosure.

In certain embodiments, the non-proliferative disease is an autoimmune disorder or an immunodeficiency disorder. In certain embodiments, the compound of Formula (I) or (II) or a pharmaceutically acceptable salt thereof, or compositions comprising such compound or pharmaceutically acceptable salt thereof, is used to prevent or treat an autoimmune disease, disorder, or condition, or an immunodeficiency disease, disorder, or condition. Exemplary autoimmune and immunodeficiency diseases, disorders, and conditions include arthritis (e.g., rheumatoid arthritis, osteoarthritis, gout), Chagas disease, chronic obstructive pulmonary disease (COPD), dermatomyositis, diabetes mellitus type 1, endometriosis, Goodpasture's syndrome, Graves' disease, Guillain-Barre syndrome (GBS), Hashiomoto's disease, Hidradenitis suppurativa, Kawasaki disease, ankylosing spondylitis, IgA nephropathy, idiopathic thrombocytopenic purpura, inflammatory bowel disease, Crohn's disease, ulcerative colitis, collagenous colitis, lymphocytic colitis, ischemic colitis, diversion colitis, Behcet's syndrome, infective colitis, indeterminate colitisinterstitial cystitis, lupus (e.g., systemic lupus erythematosus, discoid lupus, drug-induced lupus, neonatal lupus), mixed connective tissue disease, morphea, multiple sclerosis, myasthenia gravis, narcolepsy, neuromyotonia, pemphigus vulgaris, pernicious anemia, psoriasis, psoriatic arthritis, polymyositis, primary biliary cirrhosis, relapsing polychondritis, scleroderma, Sjögren's syndrome, Stiff person syndrome, vasculitis, vitiligo, a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), neutropenia, aplastic anemia, and Wegener's granulomatosis. In some embodiments, the autoimmune or immunodeficiency disorder comprises chronic mucocutaneous candidiasis. All types of autoimmune disorders and immunodeficiency disorders disclosed herein or known in the art are contemplated as being within the scope of the disclosure.

In certain embodiments, the non-proliferative disease is a cardiovascular condition. In certain embodiments, the compound of Formula (I) or (II) or a pharmaceutically acceptable salt thereof, or compositions comprising such compound or pharmaceutically acceptable salt thereof, is used to prevent or treat a cardiovascular disease, disorder, or condition. A cardiovascular disease, disorder, or condition may include a condition relating to the heart or vascular system, such as the arteries, veins, or blood. Exemplary cardiovascular diseases, disorders, or conditions include angina, arrhythmias (atrial or ventricular or both), heart failure, arteriosclerosis, atheroma, atherosclerosis, cardiac hypertrophy, cardiac or vascular aneurysm, cardiac myocyte dysfunction, carotid obstructive disease, endothelial damage after PTCA (percutaneous transluminal coronary angioplasty), hypertension including essential hypertension, pulmonary hypertension and secondary hypertension (renovascular hypertension, chronic glomerulonephritis), myocardial infarction, myocardial ischemia, peripheral obstructive arteriopathy of a limb, an organ, or a tissue; peripheral artery occlusive disease (PAOD), reperfusion injury following ischemia of the brain, heart or other organ or tissue, restenosis, stroke, thrombosis, transient ischemic attack (TIA), vascular occlusion, vasculitis, and vasoconstriction. All types of cardiovascular diseases, disorders, or conditions disclosed herein or known in the art are contemplated as being within the scope of the disclosure.

In certain embodiments, the non-proliferative disease is a metabolic disorder. In certain embodiments, the compound of Formula (I) or (II) or a pharmaceutically acceptable salt thereof, or compositions comprising such compound or pharmaceutically acceptable salt thereof, is used to prevent or treat a metabolic disease, disorder, or condition. A metabolic disease, disorder, or condition may include a disorder or condition that is characterized by abnormal metabolism, such as those disorders relating to the consumption of food and water, digestion, nutrient processing, and waste removal. A metabolic disease, disorder, or condition may include an acid-base imbalance, a mitochondrial disease, a wasting syndrome, a malabsorption disorder, an iron metabolism disorder, a calcium metabolism disorder, a DNA repair deficiency disorder, a glucose metabolism disorder, hyperlactatemia, a disorder of the gut microbiota. Exemplary metabolic conditions include obesity, diabetes (Type I or Type II), insulin resistance, glucose intolerance, lactose intolerance, eczema, hypertension, Hunter syndrome, Krabbe disease, sickle cell anemia, maple syrup urine disease, Pompe disease, and metachromatic leukodystrophy. All types of metabolic diseases, disorders, or conditions disclosed herein or known in the art are contemplated as being within the scope of the disclosure.

In certain embodiments, the non-proliferative disease is a respiratory condition. In certain embodiments, the compound of Formula (I) or (II) or a pharmaceutically acceptable salt thereof, or compositions comprising such compound or pharmaceutically acceptable salt thereof, is used to prevent or treat a respiratory disease, disorder, or condition. A respiratory disease, disorder, or condition can include a disorder or condition relating to any part of the respiratory system, such as the lungs, alveoli, trachea, bronchi, nasal passages, or nose. Exemplary respiratory diseases, disorders, or conditions include asthma, allergies, bronchitis, allergic rhinitis, chronic obstructive pulmonary disease (COPD), lung cancer, oxygen toxicity, emphysema, chronic bronchitis, and acute respiratory distress syndrome. All types of respiratory diseases, disorders, or conditions disclosed herein or known in the art are contemplated as being within the scope of the disclosure.

In certain embodiments, the non-proliferative disease is a renal disease. In certain embodiments, the compound of Formula (I) or (II) or a pharmaceutically acceptable salt thereof, or compositions comprising such compound or pharmaceutically acceptable salt thereof, is used to prevent or treat a renal disease, disorder, or condition. A renal disease, disorder, or condition can include a disease, disorder, or condition relating to any part of the waste production, storage, and removal system, including the kidneys, ureter, bladder, urethra, adrenal gland, and pelvis. Exemplary renal diseases include acute kidney failure, amyloidosis, Alport syndrome, adenovirus nephritis, acute lobar nephronia, tubular necrosis, glomerulonephritis, kidney stones, urinary tract infections, chronic kidney disease, polycystic kidney disease, and focal segmental glomerulosclerosis (FSGS). In some embodiments, the renal disease, disorder, or condition comprises HIV-associated nephropathy or hypertensive nephropathy. All types of renal diseases, disorders, or conditions disclosed herein or known in the art are contemplated as being within the scope of the disclosure.

In certain embodiments, the non-proliferative disease is an infectious disease. In certain embodiments, the compound of Formula (I) or (II) or a pharmaceutically acceptable salt thereof, or compositions comprising such compound or pharmaceutically acceptable salt thereof, is used to prevent or treat an infectious disease, disorder, or condition. An infectious disease may be caused by a pathogen such as a virus or bacteria. Exemplary infectious diseases include human immunodeficiency syndrome (HIV), acquired immunodeficiency syndrome (AIDS), meningitis, African sleeping sickness, actinomycosis, pneumonia, botulism, chlamydia, Chagas disease, Colorado tick fever, cholera, typhus, giardiasis, food poisoning, ebola hemorrhagic fever, diphtheria, Dengue fever, gonorrhea, streptococcal infection (e.g., Group A or Group B), hepatitis A, hepatitis B, hepatitis C, herpes simplex, hookworm infection, influenza, Epstein-Barr infection, Kawasaki disease, kuru, leprosy, leishmaniasis, measles, mumps, norovirus, meningococcal disease, malaria, Lyme disease, listeriosis, rabies, rhinovirus, rubella, tetanus, shingles, scarlet fever, scabies, Zika fever, yellow fever, tuberculosis, toxoplasmosis, or tularemia. In some embodiments, the infectious disease comprises cytomegalovirus. All types of infectious diseases, disorders, or conditions disclosed herein or known in the art are contemplated as being within the scope of the disclosure.

In certain embodiments, the disease, disorder, or condition is a haploinsufficiency disease. In certain embodiments, the compound of Formula (I) or (II) or a pharmaceutically acceptable salt thereof, or compositions comprising such compound or pharmaceutically acceptable salt thereof, is used to prevent or treat a haploinsufficiency disease, disorder, or condition. A haploinsufficiency disease, disorder, or condition may refer to a monogenic disease in which an allele of a gene has a loss-of-function lesion, e.g., a total loss of function lesion. In an embodiment, the loss-of-function lesion is present in an autosomal dominant inheritance pattern or is derived from a sporadic event. In an embodiment, the reduction of gene product function due to the altered allele drives the disease phenotype despite the remaining functional allele (i.e. said disease is haploinsufficient with regard to the gene in question). In an embodiment, a compound of Formula (I) or (II) increases expression of the haploinsufficient gene locus. In an embodiment, a compound of Formula (I) or (II) increases one or both alleles at the haploinsufficient gene locus. Exemplary haploinsufficiency diseases, disorders, and conditions include Robinow syndrome, cardiomyopathy, cerebellar ataxia, pheochromocytoma, Charcot-Marie-Tooth disease, neuropathy, Takenouchi-Kosaki syndrome, Coffin-Siris syndrome 2, chromosome 1p35 deletion syndrome, spinocerebellar ataxia 47, deafness, seizures, dystonia 9, GLUT1 deficiency syndrome 1, GLUT1 deficiency syndrome 2, stomatin-deficient cryohydrocytosis, basal cell carcinoma, basal cell nevus syndrome, medulloblastoma, somatic, brain malformations, macular degeneration, cone-rod dystrophy, Dejerine-Sottas disease, hypomyelinating neuropathy, Roussy-Levy syndrome, glaucoma, autoimmune lymphoproliferative syndrome, pituitary hormone deficiency, epileptic encephalopathy, early infantile, popliteal pterygium syndrome, van der Woude syndrome, Loeys-Dietz syndrome, Skraban-Deardorff syndrome, erythrocytosis, megalencephaly-polymicrogyria-polydactyly-hydrocephalus syndrome, mental retardation, CINCA syndrome, familial cold inflammatory syndrome 1, keratoendothelitis fugax hereditaria, Muckle-Wells syndrome, Feingold syndrome 1, Acute myeloid leukemia, Heyn-Sproul-Jackson syndrome, Tatton-Brown-Rahman syndrome, Shashi-Pena syndrome, Spastic paraplegia, autosomal dominant, macrophthalmia, colobomatous, with microcornea, holoprosencephaly, schizencephaly, endometrial cancer, familial, colorectal cancer, hereditary nonpolyposis, intellectual developmental disorder with dysmorphic facies and behavioral abnormalities, ovarian hyperstimulation syndrome, schizophrenia, Dias-Logan syndrome, premature ovarian failure, dystonia, dopa-responsive, due to sepiapterin reductase deficiency, Beck-Fahrner syndrome, chromosome 2p12-p11.2 deletion syndrome, neuronopathy, spastic paraplegia, familial adult myoclonic, colorectal cancer, hypothyroidism, Culler-Jones syndrome, holoprosencephaly, myelokathexis, WHIM syndrome, Mowat-Wilson syndrome, mental retardation, an intellectual developmental disorder, autism spectrum disorder, epilepsy, epileptic encephalopathy, Dravet syndrome, migraines, a mental retardation disorder (e.g., a disorder caused by a SETD5 gene mutation, e.g., intellectual disability-facial dysmorphism syndrome, autism spectrum disorder), a disorder caused by a GATA2 mutation (e.g., GATA2 deficiency; GATA2 haploinsufficiency; Emberger syndrome; monocytopenia and Mycobacterium avium complex/dendritic cell, monocyte, B and NK lymphocyte deficiency; familial myelodysplastic syndrome; acute myeloid leukemia; chronic myelomonocytic leukemia), and febrile seizures.

In certain embodiments, the disease, disorder, or condition is an autosomal recessive disease, e.g., with residual function. In certain embodiments, the compound of Formula (I) or (II), or a pharmaceutically acceptable salt thereof, or compositions comprising such compound or pharmaceutically acceptable salt thereof, is used to prevent or treat an autosomal recessive disease, disorder, or condition. An autosomal recessive disease with residual function may refer to a monogenic disease with either homozygous recessive or compound heterozygous heritability. These diseases may also be characterized by insufficient gene product activity (e.g., a level of gene product greater than 0%). In an embodiment, a compound of Formula (I) or (II) may increase the expression of a target (e.g., a gene) related to an autosomal recessive disease with residual function. Exemplary autosomal recessive diseases with residual function include Friedreich's ataxia, Stargardt disease, Usher syndrome, chlorioderma, fragile X syndrome, achromatopsia 3, Hurler syndrome, hemophilia B, alpha-1-antitrypsin deficiency, Gaucher disease, X-linked retinoschisis, Wiskott-Aldrich syndrome, mucopolysaccharidosis (Sanfilippo B), DDC deficiency, epidermolysis bullosa dystrophica, Fabry disease, metachromatic leukodystrophy, and odontochondrodysplasia.

In certain embodiments, the disease, disorder, or condition is an autosomal dominant disease. In certain embodiments, the compound of Formula (I) or (II), or a pharmaceutically acceptable salt thereof, or compositions comprising such compound or pharmaceutically acceptable salt thereof, is used to prevent or treat an autosomal dominant disease, disorder, or condition. An autosomal dominant disease may refer to a monogenic disease in which the mutated gene is a dominant gene. These diseases may also be characterized by insufficient gene product activity (e.g., a level of gene product greater than 0%). In an embodiment, a compound of Formula (I) or (II) may increase the expression of a target (e.g., a gene) related to an autosomal dominant disease. Exemplary autosomal dominant diseases include Huntington's disease, achondroplasia, antithrombin III deficiency, Gilbert's disease, Ehlers-Danlos syndrome, hereditary hemorrhagic telangiectasia, intestinal polyposis, hereditary elliptosis, hereditary spherocytosis, marble bone disease, Marfan's syndrome, protein C deficiency, Treacher Collins syndrome, Von Willebrand's disease, tuberous sclerosis, osteogenesis imperfecta, polycystic kidney disease, neurofibromatosis, and idiopathic hypoparathyroidism.

In certain embodiments, the disease, disorder, or condition is a paralogue activation disorder. In certain embodiments, the compound of Formula (I) or (II), or a pharmaceutically acceptable salt thereof, or compositions comprising such compound or pharmaceutically acceptable salt thereof, is used to prevent or treat a paralogue activation disease, disorder, or condition. A paralogue activation disorder may comprise a homozygous mutation of genetic locus leading to loss-of-function for the gene product. In these disorders, there may exist a separate genetic locus encoding a protein with overlapping function (e.g. developmental paralogue), which is otherwise not expressed sufficiently to compensate for the mutated gene. In an embodiment, a compound of Formula (I) or (II) activates a gene connected with a paralogue activation disorder (e.g., a paralogue gene).

The cell described herein may be an abnormal cell. The cell may be in vitro or in vivo. In certain embodiments, the cell is a proliferative cell. In certain embodiments, the cell is a cancer cell. In certain embodiments, the cell is a non-proliferative cell. In certain embodiments, the cell is a blood cell. In certain embodiments, the cell is a lymphocyte. In certain embodiments, the cell is a benign neoplastic cell. In certain embodiments, the cell is an endothelial cell. In certain embodiments, the cell is an immune cell. In certain embodiments, the cell is a neuronal cell. In certain embodiments, the cell is a glial cell. In certain embodiments, the cell is a brain cell. In certain embodiments, the cell is a fibroblast. In certain embodiment, the cell is a primary cell, e.g., a cell isolated from a subject (e.g., a human subject).

In some embodiments, a compound of Formula (I) or (II) or a pharmaceutically acceptable salt thereof, e.g., as described herein, has improved cell permeability over a reference compound, e.g., in a standard assay for measuring cell permeability. Cell permeability may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell permeability measurement (Papp) of <2×10⁻⁶ cm s⁻¹. In an embodiment, a compound of Formula (I) or (II) or a pharmaceutically acceptable salt thereof, e.g., as described herein, has a cell permeability measurement (Papp) of between 2-6×10⁻⁶ cm s⁻¹. In an embodiment, a compound of Formula (I) or (II) or a pharmaceutically acceptable salt thereof, e.g., as described herein, has a cell permeability measurement (Papp) of Papp greater than 6×10⁻⁶ cm s⁻¹. In an embodiment, a compound of Formula (I) or (II) or a pharmaceutically acceptable salt thereof, e.g., as described herein, has a cell permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits decreased cell efflux, e.g., over a reference compound, e.g., in a standard assay for measuring cell efflux. Cell efflux may be investigated, for example, using a standard assay run in either Madin-Darby Canine Kidney (MDCK) cells expressing Breast Cancer Resistance Protein (BCRP) or subclone MDCKII cells expressing Multidrug Resistance Protein 1 (MDR1); see, e.g., Drug Metabolism and Disposition 36, 268-275 (2008) and Journal of Pharmaceutical Sciences 107 2225-2235 (2018). In an embodiment, a compound of Formula (I) or (II) or a pharmaceutically acceptable salt thereof, e.g., as described herein, has a cell efflux ratio of less than 1.5. In an embodiment, a compound of Formula (I) or (II) or a pharmaceutically acceptable salt thereof, e.g., as described herein, has a cell efflux ratio of between 1.5 and 5. In an embodiment, a compound of Formula (I) or (II) or a pharmaceutically acceptable salt thereof, e.g., as described herein, has a cell efflux ratio greater than 5. In an embodiment, a compound of Formula (I) or (II) or a pharmaceutically acceptable salt thereof, e.g., as described herein, has a cell efflux ratio less than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the expression of a target protein (e.g., HTT or MYB) in a reference cell or sample. In an embodiment, a compound of Formula (I) or (II) or a pharmaceutically acceptable salt thereof, e.g., as described herein, increases the expression of a target protein (e.g., HTT or MYB) in a reference cell or sample. In an embodiment, a compound of Formula (I) or (II) or a pharmaceutically acceptable salt thereof, e.g., as described herein, decreases the expression of a target protein (e.g., HTT or MYB) in a reference cell or sample. The effect of an exemplary compound of Formula (I) or (II) on protein abundance may be measured using a standard assay for measuring protein abundance, such as the HiBit-assay system (Promega). In this assay, percent response for each respective cell line may be as calculated at each compound concentration as follows: % response=100*(S−PC)/(NC−PC). For the normalized response at each concentration, a four-parameter logistical regression may be fit to the data and the response may be interpolated at the 50% value to determine a concentration for protein abundance at 50% (IC₅₀) an untreated control. In an embodiment, a compound of Formula (I) or (II) or a pharmaceutically acceptable salt thereof, e.g., as described herein, has a protein abundance response less than 100 nM. In an embodiment, a compound of Formula (I) or (II) or a pharmaceutically acceptable salt thereof, e.g., as described herein, has a protein abundance response between 100-1000 nM. In an embodiment, a compound of Formula (I) or (II) or a pharmaceutically acceptable salt thereof, e.g., as described herein, has a protein abundance response greater than 1000 nM. In an embodiment, a compound of Formula (I) or (II) or a pharmaceutically acceptable salt thereof, e.g., as described herein, has a protein abundance response greater than 10 uM. In an embodiment, a compound of Formula (I) or (II) or a pharmaceutically acceptable salt thereof, e.g., as described herein, modulates the protein abundance of a target protein by about 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, modulates the viability of a target cell in a subject or sample. In an embodiment, a compound of Formula (I) or (II) or a pharmaceutically acceptable salt thereof, e.g., as described herein, increases the viability of a target cell in a subject or sample. In an embodiment, a compound of Formula (I) or (II) or a pharmaceutically acceptable salt thereof, e.g., as described herein, decreases the viability of a target cell in a subject or sample. In an embodiment, a compound of Formula (I) or (II) or a pharmaceutically acceptable salt thereof, e.g., as described herein, does not impact the viability of a cell (e.g., is non-toxic) in a subject or sample. The effect an exemplary compound of Formula (I) or (II) on cell viability may be measured using a standard assay for measuring cell toxicity, such as the Cell Titer Glo 2.0 assay in either K562 (human chronic myelogenous leukemia) or SH-SY5Y (human neuroblastoma) cells. The concentration at which cell viability is measured may be based on the particular assay used. In an embodiment, a compound of Formula (I) or (II) or a pharmaceutically acceptable salt thereof, e.g., as described herein, is tolerated by a target cell at a concentration of less than 100 nM. In an embodiment, a compound of Formula (I) or (II) or a pharmaceutically acceptable salt thereof, e.g., as described herein, is tolerated by a target cell at a concentration of between 100-1000 nM. In an embodiment, a compound of Formula (I) or (II) or a pharmaceutically acceptable salt thereof, e.g., as described herein, is tolerated by a target cell at a concentration of greater than 1000 nM. In an embodiment, a compound of Formula (I) or (II) or a pharmaceutically acceptable salt thereof, e.g., as described herein, is tolerated by a target cell at a concentration of greater than 10 uM.

In some embodiments, a compound of Formula (I) or (II) or a pharmaceutically acceptable salt thereof, e.g., as described herein, has improved brain permeability over a reference compound, e.g., in a standard assay for measuring brain permeability. Brain permeability may be measured, for example, by determining the unbound partition coefficient (Kpuu), brain. In such an assay, the unbound brain partition coefficient (K_{p,uu,bain}) may be defined as the ratio of unbound brain-free compound concentration to unbound plasma concentration. It is calculated using the following equation: K_{p,uu,bain}=f_{u,brain}×C_{brain}/f_{u,plasma}×Cₚₗₐₛₘₐ. C_{brain} and Cₚₗₐₛₘₐ represent the total concentrations in brain and plasma, respectively. In this assay, the f_{u,brain} and f_{u,plasma} may be the unbound fraction of the compound in brain and plasma, respectively. Both f_{u,brain} and f_{u,plasma} may be determined in vitro via equilibrium dialysis. In an embodiment, a compound of Formula (I) or (II) or a pharmaceutically acceptable salt thereof, e.g., as described herein, has a Kp value of greater than 5. In an embodiment, a compound of Formula (I) or (II) or a pharmaceutically acceptable salt thereof, e.g., as described herein, has a Kp value between 1 and 5. In an embodiment, a compound of Formula (I) or (II) or a pharmaceutically acceptable salt thereof, e.g., as described herein, has a Kp value between 0.2-1. In an embodiment, a compound of Formula (I) or (II) or a pharmaceutically acceptable salt thereof, e.g., as described herein, has a Kp value of less than 0.2. In an embodiment, a compound of Formula (I) or (II) or a pharmaceutically acceptable salt thereof, e.g., as described herein, has a Kpuu value of greater than 2.5. In an embodiment, a compound of Formula (I) or (II) or a pharmaceutically acceptable salt thereof, e.g., as described herein, has a Kpuu value between 0.5-2.5. In an embodiment, a compound of Formula (I) or (II) or a pharmaceutically acceptable salt thereof, e.g., as described herein, has a Kpuu value between 0.1-0.5. In an embodiment, a compound of Formula (I) or (II) or a pharmaceutically acceptable salt thereof, e.g., as described herein, has a Kpuu value of less than 0.1. In an embodiment, a compound of Formula (I) or (II) or a pharmaceutically acceptable salt thereof, e.g., as described herein, has a brain permeability greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a reference compound.

In some embodiments, a compound of Formula (I) or (II) or a pharmaceutically acceptable salt thereof, e.g., as described herein, exhibits selectivity for one target nucleic acid sequence, e.g., pre-mRNA transcript sequence or bulge, compared to another target nucleic acid sequence, e.g., pre-mRNA transcript sequence or bulge. In an embodiment, a compound of Formula (I) or (II) or a pharmaceutically acceptable salt thereof, e.g., as described herein, exhibits selectivity for HTT, e.g., an HTT-related nucleic acid sequence. In an embodiment, a compound of Formula (I) or (II) or a pharmaceutically acceptable salt thereof, e.g., as described herein, exhibits selectivity for SMN2, e.g., an SMN2-related nucleic acid sequence. In an embodiment, a compound of Formula (I) or (II) or a pharmaceutically acceptable salt thereof, e.g., as described herein, exhibits selectivity for Target C, e.g., a Target C-related nucleic acid sequence. In an embodiment, a compound of Formula (I) or (II) or a pharmaceutically acceptable salt thereof, e.g., as described herein, exhibits selectivity for MYB, e.g., a MYB-related nucleic acid sequence. Selectivity for one target nucleic acid sequence over another may be measured using any number of methods known in the art. In an embodiment, selectivity may be measured by determining the ratio of derived qPCR values (e.g., as described herein) for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for one target nucleic acid sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over another target nucleic acid sequence. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for Target C sequence over another. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for HTT over SMN2. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over HTT. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for SMN2 over MYB. In an embodiment, a compound of Formula (I) or (II) or a pharmaceutically acceptable salt thereof, e.g., as described herein, has a ratio of greater than 1.1, 1.5, 2, 3, 4, 5, 6, 7, 8, 9, 10, 15, 20, 25, 30, 35, 40, 45, 50, 75, or 100 selectivity for MYB over SMN2. In an embodiment, a compound of Formula (I) or (II) or a pharmaceutically acceptable salt thereof, e.g., as described herein, has a 3-fold greater selectivity for HTT over MYB. In an embodiment, a compound of Formula (I) or (II) or a pharmaceutically acceptable salt thereof, e.g., as described herein, has a 3-fold greater selectivity for MYB over HTT. In an embodiment, a compound of Formula (I) or (II) or a pharmaceutically acceptable salt thereof, e.g., as described herein, has a 10-fold greater selectivity for HTT over MYB. In an embodiment, a compound of Formula (I) or (II) or a pharmaceutically acceptable salt thereof, e.g., as described herein, has a 10-fold greater selectivity for MYB over HTT. In an embodiment, a compound of Formula (I) or (II) or a pharmaceutically acceptable salt thereof, e.g., as described herein, has a 3-fold greater selectivity for HTT over SMN2. In an embodiment, a compound of Formula (I) or (II) or a pharmaceutically acceptable salt thereof, e.g., as described herein, has a 3-fold greater selectivity for SMN2 over HTT. In an embodiment, a compound of Formula (I) or (II) or a pharmaceutically acceptable salt thereof, e.g., as described herein, has a 10-fold greater selectivity for HTT over SMN2. In an embodiment, a compound of Formula (I) or (II) or a pharmaceutically acceptable salt thereof, e.g., as described herein, has a 10-fold greater selectivity for SMN2 over HTT. In an embodiment, a compound of Formula (I) or (II) or a pharmaceutically acceptable salt thereof, e.g., as described herein, has a 3-fold greater selectivity for MYB over SMN2. In an embodiment, a compound of Formula (I) or (II) or a pharmaceutically acceptable salt thereof, e.g., as described herein, has a 3-fold greater selectivity for SMN2 over MYB. In an embodiment, a compound of Formula (I) or (II) or a pharmaceutically acceptable salt thereof, e.g., as described herein, has a 10-fold greater selectivity for MYB over SMN2. In an embodiment, a compound of Formula (I) or (II) or a pharmaceutically acceptable salt thereof, e.g., as described herein, has a 10-fold greater selectivity for SMN2 over MYB. In an embodiment, a compound of Formula (I) or (II) or a pharmaceutically acceptable salt thereof, e.g., as described herein, has a selectivity for one target nucleic acid sequence that is greater than 1%, 5%, 10%, 15%, 20%, 25%, 30%, 35%, 40%, 45%, 50%, 55%, 60%, 65%, 70%, 75%, 80%, 85%, 90%, 95%, 99% or more, e.g., compared with a second nucleic acid sequence.

In certain embodiments, the methods described herein comprise the additional step of administering one or more additional pharmaceutical agents in combination with the compound of Formula (I) or (II), a pharmaceutically acceptable salt thereof, or compositions comprising such compound or pharmaceutically acceptable salt thereof. Such additional pharmaceutical agents include, but are not limited to, anti-proliferative agents, anti-cancer agents, anti-diabetic agents, anti-inflammatory agents, immunosuppressant agents, and a pain-relieving agent. The additional pharmaceutical agent(s) may synergistically augment the modulation of splicing induced by the inventive compounds or compositions of this disclosure in the biological sample or subject. Thus, the combination of the inventive compounds or compositions and the additional pharmaceutical agent(s) may be useful in treating, for example, a cancer or other disease, disorder, or condition resistant to a treatment using the additional pharmaceutical agent(s) without the inventive compounds or compositions.

### EXAMPLES

In order that the invention described herein may be more fully understood, the following examples are set forth. The examples described in this application are offered to illustrate the compounds, pharmaceutical compositions, and methods provided herein and are not to be construed in any way as limiting their scope.

The compounds provided herein can be prepared from readily available starting materials using modifications to the specific synthesis protocols set forth below that would be well known to those of skill in the art. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvents used, but such conditions can be determined by those skilled in the art by routine optimization procedures.

Additionally, as will be apparent to those skilled in the art, conventional protecting groups may be necessary to prevent certain functional groups from undergoing undesired reactions. The choice of a suitable protecting group for a particular functional group as well as suitable conditions for protection and deprotection are well known in the art. For example, numerous protecting groups, and their introduction and removal, are described in Greene et al., Protecting Groups in Organic Synthesis, Second Edition, Wiley, New York, 1991, and references cited therein.

Reactions can be purified or analyzed according to any suitable method known in the art. For example, product formation can be monitored by spectroscopic means, such as nuclear magnetic resonance (NMR) spectroscopy (e.g., ¹H or ¹³C), infrared (IR) spectroscopy, spectrophotometry (e.g., UV-visible), mass spectrometry (MS), or by chromatographic methods such as high performance liquid chromatography (HPLC) or thin layer chromatography (TLC). In some embodiments, absolute stereochemistry of chiral compounds provided herein is arbitrarily assigned.

Proton NMR: ¹H NMR spectra were recorded in CDCl₃ solution in 5-mm o.d. tubes (Wildmad) at 24° C. and were collected on a BRUKER AVANCE NEO 400 at 400 MHz for ¹H. The chemical shifts (δ) are reported relative to tetramethylsilane (TMS=0.00 ppm) and expressed in ppm.

LC/MS: Liquid chromatography-mass spectrometry (LC/MS) was performed on Shimadzu-2020EV using column: Shim-pack XR-ODS (C18, Ø4.6×50 mm, 3 m, 120 Å, 40° C.) operating in ESI(+) ionization mode; flow rate=1.2 mL/min. Mobile phase=0.05% TFA in water or CH₃CN; or on Shimadzu-2020EV using column: Poroshell HPH—C18 (C18, Ø4.6×50 mm, 3 m, 120 Å, 40° C.) operating in ESI(+) ionization mode; flow rate=1.2 mL/min. Mobile phase A: Water/5 mM NH₄HCO₃, Mobile phase B: CH₃CN.)

Reverse flash chromatography: Column: C18 silica gel.
Condition 1: Mobile Phase A: water; Mobile Phase B: methanol. Gradient 1: 10% B to 50% B in 10 min.
Condition 2: Mobile Phase A: methanol; Mobile Phase B: dichloromethane. Gradient 1: 0% B to 10% B in 10 min.
Condition 3: Mobile Phase A: water (10 mmol/L NH₄HCO₃); Mobile Phase B: acetonitrile; Gradient 1: 20% B to 40% B in 30 min; Gradient 2: 10% B to 30% B in 20 min; Gradient 3: 10% B to 50% B in 10 min; Gradient 4: 10% B to 55% B in 10 min; Gradient 5: 20% B to 70% B in 12 min; Gradient 6: 10% B to 80% B in 25 min; Gradient 7: 30% B to 80% B in 30 min.
Condition 4: Mobile Phase A: water (0.1% TFA); Mobile Phase B: acetonitrile; Gradient 1: 12% B to 51% B in 12 min; Gradient 2: 10% B to 55% B in 12 min; Gradient 3: 20% B to 60% B in 10 min; Gradient 4: 10% B to 50% B in 10 min; Gradient 5: 10% B to 30% B in 10 min; Gradient 6: 20% B to 40% B in 10 min; Gradient 7: 30% B to 80% B in 12 min.
Condition 5: Column: YMC-Actus Triart C18, 30×150 mm, 5 m; Mobile Phase A: Water (10 mmol/L NH₄HCO₃), Mobile Phase B: ACN; Flow rate: 60 mL/min; Gradient 1: 10% B to 60% B in 8 min; Gradient 2: 20% B to 60% B in 8 min; Gradient 3: 10% B to 50% B in 8 min; Gradient 4: 25% B to 58% B in 8 min; Gradient 5: 15% B to 71% B in 8 min.
Condition 6: Column: Xselect CSH C18 OBD Column 30×150 mm 5 m; Mobile Phase A: Water (10 mmol/L NH₄HCO₃), Mobile Phase B: ACN; Flow rate: 60 mL/min.
Condition 7: Column: C18 silica gel; mobile phase, acetonitrile in water (0.1% FA), Gradient 1: 20% to 70% gradient in 12 min; Gradient 2: 20% to 65% gradient in 12 min; Gradient 3: 20% B to 50% B in 10 min; Gradient 4: 30% to 70% gradient in 12 min; Gradient 5: 30% B to 80% B in 10 min; Gradient 6: 60% B to 95% B in 12 min; Gradient 7: 5% B to 35% B in 12 min.
Condition 8: Column, C18; mobile phase, MeCN in water (10 mmol/L NH₄HCO₃); Gradient 1: 10% B to 50% B in 10 min.
Condition 9: Column, C18 silica gel; Mobile Phase A: water (0.1% NH₃·H₂O+10 mmol/L NH₄HCO₃), Mobile Phase B: Acetonitrile; Gradient 1: 10% B to 50% B in 12 min; Gradient 2: 20% B to 80% B in 10 min; Gradient 3: 5% B to 30% B in 10 min; Gradient 4: 20% B to 65% B in 10 min; Gradient 5: 30% B to 80% B in 12 min; Gradient 6: 40% B to 90% B in 12 min; Gradient 7: 15% B to 60% B in 12 min; Gradient 8: 20% B to 70% B in 12 min; Gradient 9: 30% B to 80% B in 10 min.
Condition 10: Column, C18 silica gel; Mobile Phase A: Water (0.1% NH₃·H₂O), Mobile Phase B: Acetonitrile; Gradient 1: 20% B to 70% B in 10 min; Gradient 2: 15% B to 50% B in 10 min; Gradient 3: 10% B to 60% B in 10 min; Gradient 4: 20% B to 60% B in 10 min; Gradient 5: 10% B to 70% B in 10 min; Gradient 6: 30% B to 60% B in 10 min; Gradient 7: 30% B to 70% B in 10 min; Gradient 8: 30% B to 80% B in 10 min; Gradient 9: 20% B to 40% B in 10 min; Gradient 10: 10% B to 50% B in 10 min; Gradient 11: 40% B to 90% B in 12 min; Gradient 12: 20% B to 60% B in 12 min; Gradient 13: 10% B to 55% B in 12 min; Gradient 14: 10% B to 65% B in 10 min; Gradient 15: 30% B to 50% B in 10 min; Gradient 16: 50% B to 85% B in 8 min; Gradient 17: 30% B to 80% B in 12 min; Gradient 18: 40% B to 60% B in 10 min; Gradient 19:20% B to 50% B in 10 min; Gradient 20: 12% B to 67% B in 12 min; Gradient 21: 20% B to 90% B in 12 min; Gradient 22: 20% B to 65% B in 12 min; Gradient 23: 10% B to 50% B in 12 min; Gradient 24: 20% B to 70% B in 12 min; Gradient 25: 40% B to 90% B in 10 min; Gradient 26: 5% B to 55% B in 10 min; Gradient 27: 10% B to 55% B in 10 min.
Condition 11: Column: XBridge BEH C18 OBD, 19×250 mm, 5 um; Mobile Phase A: water (10 mmol/L NH₄HCO₃), Mobile Phase B: ACN; Gradient 1: 10% B to 50% B in 8 min.
Condition 12: Column, C18 silica gel; Mobile Phase A: Water (0.1% TFA), Mobile Phase B: MeOH; Gradient 1: 10% B to 50% B in 10 min.
Condition 13: Column, C18 silica gel; Mobile Phase A: water (0.1% NH₃·H₂O), Mobile Phase B: MeOH; Gradient 1: 10% B to 50% B in 10 min.
Condition 14: Column, C18 silica gel; mobile phase A: water (0.1% HCl), Mobile Phase B: acetonitrile; Gradient 1: 20% B to 70% B in 12 min.
Analytical chiral HPLC: Analytical chiral HPLC was performed on a Agilent 1260 using column: CHLRALPAK IG-3, CHIRALPAK IC-3 or CHIRALPAK OJ-3, with flow rate=1.2 mL/min. Mobile phase=MTBE(DEA):EtOH=:50:50).
Preparative HPLC purification: prep-HPLC purification was performed on a Waters-2545 or Shimadzu, using column: X-Select CSH C18 OBD (130 Å, 5 μm, 30 mm×150 mm), XBridge Prep OBD C18 (30×150 mm, 5 μm), XBridge Prep C18 OBD (Sum, 19 mm×150 mm), XBridge Shield RP18 OBD (19*150 mm, 5 μm), or Kinetex EVO C18 Column (30*150 mm, m).
Condition 1: Column: XBridge Prep OBD C18, 30×150 mm, 5 μm; Mobile Phase A: water (10 mmol/L NH₄HCO₃); Mobile Phase B: acetonitrile; Flow rate: 60 mL/min; Gradient 1: 45% B to 95% B in 8 min; Gradient 2: 5% B to 50% B in 8 min; Gradient 3: 5% B to 45% B in 8 min; Gradient 4: 5% B to 55% B in 8 min; Gradient 5: 5% B to 42% B in 8 min; Gradient 6: 5% B to 35% B in 8 min; Gradient 7: 5% B to 30% B in 8 min; Gradient 8: 20% B to 66% B in 8 min; Gradient 9: 10% B to 55% B in 8 min; Gradient 10: 5% B to 40% B in 8 min; Gradient 11: 10% B in 50% B in 8 min; Gradient 12: 20% B to 50% B in 8 min; Gradient 13: 25% B to 65% B in 8 min; Gradient 14: 5% B to 85% B in 8 min; Gradient 15: 10% B to 40% B in 8 min; Gradient 16: 5% B to 37% B; Gradient 17: 5% B to 38% B in 8 min; Gradient 18: 5% B to 45% B; Gradient 19: 10% B to 46% B in 8 min; Gradient 20: 10% B to 45% B in 10 min; Gradient 21: 15% B to 60% B in 8 min; Gradient 22: 35% B to 55% B in 10 min; Gradient 23: 10% B to 55% B in 8 min; Gradient 24: 20% B to 65% B in 8 min; Gradient 25: 25% B to 70% B in 8 min; Gradient 26: 10% B to 30% B in 10 min; Gradient 27: 30% B to 70% B in 8 min; Gradient 28: 10% B to 60% B in 8 min; Gradient 29: 15% B to 70% B in 8 min; Gradient 30: 25% B to 75% B in 8 min; Gradient 31: 35% B to 80% B in 8 min; Gradient 32: 30% B to 80% B in 8 min; Gradient 33: 15% B to 75% B in 8 min.
Condition 2: Column: Xselect CSH OBD; Mobile Phase A: water (10 mmol/L NH₄HCO₃); Mobile Phase B: acetonitrile; Gradient 1: 5% B to 35% B in 8 min; Gradient 2: 10% B to 50% B in 8 min; Gradient 3: 5% B to 45% B in 8 min; Gradient 4: 10% B to 90% B in 8 min; Gradient 5: 15% B to 75% B in 8 min; Gradient 6: 25% B to 75% B in 8 min; Gradient 7: 15% B to 50% B in 8 min; Gradient 8: 3% B to 20% B in 8 min; Gradient 9: 5% B to 65% B in 8 min; Gradient 10: 45% B to 75% B in 8 min; Gradient 11: 30% B to 80% B in 8 min; Gradient 12: 20% B to 80% B in 8 min; Gradient 13: 15% B to 60% B in 8 min; Gradient 14: 5% B to 30% B in 8 min.
Condition 3: Column: YMC-Actus Triart C18, 30×150 mm, 5 m; Mobile Phase A: water (10 mmol/L NH₄HCO₃); Mobile Phase B: acetonitrile; Flow rate: 60 mL/min; Gradient 1: 10% B to 65% B in 8 min; Gradient 2: 5% B to 60% B in 8 min; Gradient 3: 5% B to 43% B in 8 min; Gradient 4: 5% B to 40% B in 8 min; Gradient 5: 5% B to 38% B in 8 min; Gradient 6: 5% B to 35% B in 8 min; Gradient 7: 5% B to 37% B in 8 min; Gradient 8: 5% B to 50% B in 8 min; Gradient 9: 5% B to 75% B in 8 min; Gradient 10: 5% B to 30% B in 8 min; Gradient 11: 3% B to 3% B in 2 min; Gradient 12: 3% B to 43% B in 8 min; Gradient 13: 15% B to 65% B in 8 min; Gradient 14: 40% B to 76% B in 8 min; Gradient 15: 10% B to 45% B in 8 min; Gradient 16: 5% B to 55% B in 8 min; Gradient 17: 5% B to 45% B in 8 min; Gradient 18: 10% B to 50% B in 8 min; Gradient 19: 5% B to 57% B in 8 min; Gradient 20: 15% B to 80% B in 8 min; Gradient 21: 10% B to 85% B in 8 min; Gradient 22: 20% B to 63% B in 8 min; Gradient 23: 30% B to 75% B in 10 min; Gradient 24: 30% B to 85% B in 8 min; Gradient 25: 35% B to 78% B in 8 min.
Condition 4: Column: Xselect CSH OBD; Mobile Phase A: water (0.05% HCl); Mobile Phase B: acetonitrile; Gradient 1: 5% B to 30% B in 8 min; Gradient 2: 10% B to 50% B in 8 min; Gradient 3: 3% B to 43% B in 6 min; Gradient 4: 10% B to 60% B in 8 min.
Condition 5: Column: SunFire Prep C18 OBD Column 19*150 mm, 5 m 10 nm; Mobile Phase A: water (0.05% TFA), Mobile Phase B: acetonitrile; Gradient 1: 10% to 20% B in 7 min.
Condition 6: Column: C18 silica gel, XBridge, 19×150 mm; Mobile Phase A: water (0.05% NH₃H₂O), Mobile Phase B: acetonitrile; Gradient 1: 30% to 70% B in 7 min; Gradient 2: 24% B to 54% B in 7 min; Gradient 3: 25% B to 55% B in 7 min; Gradient 4: 20% B to 58% B in 8 min; Gradient 5: 22% B to 62% B in 8 min; Gradient 6: 70% B to 90% B in 14 min; Gradient 7: 35% B to 55% B in 7 min; Gradient 8: 40% B to 65% B in 7 min; Gradient 9: 45% B to 70% B in 7 min; Gradient 10: 45% B to 65% B in 8 min; Gradient 11: 20% B to 40% B in 7 min; Gradient 12: 50% B to 70% B in 7 min; Gradient 13: 65% B to 85% B in 7 min; Gradient 14: 60% B to 85% B in 7 min; Gradient 15: 65% B to 90% B in 8 min; Gradient 16: 63% to 85% gradient in 8 min; Gradient 17: 20% B to 45% B in 7 min; Gradient 18: 38% B to 58% B in 7 min.
Condition 7: Column: Welch Ultimate XB—C18 50*250 mm 10 um; Mobile Phase A: water (0.1% TFA), Mobile Phase B: acetonitrile; Flow rate: 90 mL/min; Gradient 1: 12% B to 47% B in 12 min; Gradient 2: 10% B to 45% B in 12 min; Gradient 3: 5% B isocratic for 2 min, 5% B to 30% B 12 min; Gradient 4: 20% B to 60% B in 12 min; Gradient 5: 25% B to 65% B in 12 min.
Condition 8: Column: SunFire Prep C18 OBD 19*150 mm, 5 m 10 nm, Mobile Phase A: water (0.05% HCl), Mobile Phase B: acetonitrile; Gradient 1: 10% to 20% B in 12 min; Gradient 2: 48% B to 72% B in 12 min; Gradient 3: 10% B to 20% B in 7 min.
Condition 9: Column: YMC-Actus Triart C18, 30×150 mm, 5 m; Mobile Phase A: water (0.05% HCl); Mobile Phase B: Acetonitrile; Flow rate: 60 mL/min; Gradient 1: 10% B to 60% B in 8 min.
Condition 10: Column: Xselect CSH OBD; Mobile Phase A: water (0.1% FA); Mobile Phase B: acetonitrile; Gradient 1: Hold 3% B for 2 min to 27% B in 6 min.
Condition 11: Xbridge Shield RP18 OBD Column, 30×150 mm, 5 m; Mobile Phase A: Water (10 mmol/L NH₄HCO₃), Mobile Phase B: acetonitrile; Flow rate: 60 mL/min; Gradient 1: 5% B to 40% B in 10 min; Gradient 2: 10% B to 45% B in 10 min; Gradient 3: 10% B to 60% B in 8 min; Gradient 4: 20% B to 75% B in 8 min; Gradient 5: 20% B to 55% B in 10 min.
Condition 12: Column: Xselect CSH OBD; Mobile Phase A: water (0.05% HCl); Mobile Phase B: acetonitrile; Gradient 1: 5% B to 25% B in 8 min.
Condition 13: Column: XBridge Prep OBD Column 19×150 mm, 8 μm; Mobile Phase A: water (0.1% NH₃·H₂O), Mobile Phase B: methanol; Flow rate: 20 mL/min; Gradient 1: 30% B to 80% B in 8 min; Gradient 2: 35% B to 75% B in 8 min; Gradient 3: 40% B to 80% B in 8 min; Gradient 4: 30% B to 60% B in 8 min; Gradient 5: 30% B to 70% B in 8 min; Gradient 6: 20% B to 75% B in 8 min.
Condition 14: SunFire Prep Column 19*150 mm, 10 nm; Mobile Phase A: water (0.05% NH₄OH), Mobile Phase B: acetonitrile; Gradient 1: 35% B to 50% B in 7 min.
Condition 15: SunFire Prep Column 19*150 mm, 10 nm; Mobile Phase A: water (0.05% NH₄OH), Mobile Phase B: acetonitrile; Gradient 1: 35% B to 50% B in 7 min.
Condition 16: Column: XBridge Shield RP18 OBD Column, 19*150 mm, 5 m; Mobile Phase A: water (10 mmol/L NH₄HCO₃), Mobile Phase B: ACN; Gradient 1: 20% B to 45% B in 8 min; Gradient 2: 15% B to 46% B in 8 min.
Condition 17: Column: Kinetex EVO C18 Column, 30*150, 5 m; Mobile Phase A: water (10 mmol/L NH₄HCO₃), Mobile Phase B: ACN; Gradient 1: 5% B to 35% B in 10 min; Gradient 2: 30% B to 65% B in 8 min.
Condition 18: Column: CHIRALPAK IF-3, 4.6*50 mm, 3 um; Mobile Phase A: MtBE(0.1% DEA), Mobile Phase B, MeOH=85: 15; Flow rate: 1 mL/min; Gradient 1: 0% B to 0% B Condition 19: Column: C18 silica gel; Mobile Phase A: water (0.1% NH₃·H₂O+10 mmol/L NH₄HCO₃), Mobile Phase B: acetonitrile; Gradient 1: 30% B to 80% B in 15 min.
Condition 20: Column, C18 silica gel, XBridge, 19×150 mm; Mobile Phase A: water (0.05% NH₄HCO₃), Mobile Phase B: acetonitrile; Gradient 1: 60% B to 80% B in 7 min; Preparative chiral HPLC: purification by chiral HPLC was performed on a Gilson-GX 281 using column: CHIRALPAK IG-3, CHIRALPAK IC-3 or CHIRALPAK OJ-3, YMC-Actus Triart CIS, Xselect CSH C18 OBD, XBridge Prep OBD CIS, or XBridge Shield RP18 OBD.
Condition 1: Column: CHIRAL ART Cellulose-SB, 2×25 cm, 5 m; Mobile Phase A: MtBE (0.1% DEA); Mobile Phase B: EtOH; Flow rate: 20 mL/min; Gradient 1: 30% B to 30% B in 7 min; Gradient 2: 15% B to 15% B in 7 min; Gradient 3: 0% B to 0% B; Gradient 4: 15% B to 15% B in 10 min; Gradient 5: 15% B to 15% B in 8 min; Gradient 6: 15% B to 15% B in 8.5 min; Gradient 7: 30% B to 30% B in 7 min; Gradient 8: 30% B to 30% B in 6.5 min; Gradient 9: 30% B to 30% B in 10.5 min; Gradient 10: 15% B to 15% B in 11 min; Gradient 11: 20% B to 20% B in 7.5 min; Gradient 12: 20% B to 20% B in 6.5 min; Gradient 13: 10% B to 10% B in 7.5 min; Gradient 14: 30% B isocratic in 11 min.
Condition 2: Column: CHIRALPAK ID, 2*25 cm, 5 m; Mobile Phase A: MtBE (0.1% DEA); Mobile Phase B: EtOH; Flow rate 1: 20 mL/min; Flow rate 2: Gradient 1: 20% B to 20% B in 15 min; Gradient 2: 0% B to 0% B.
Condition 3: Column: CHIRALPAK IA-3, 4.6*50 mm, 3 μm; Mobile Phase A: MtBE (0.1% DEA): EtOH=80: 20; Flow rate: 1 mL/min; Gradient 1: 0% B to 0% B; Gradient 2: 50% B isocratic in 20 min
Condition 4: Column: CHIRAL ART Cellulose-SB, 2×25 cm, 5 m; Mobile Phase A: MtBE (0.1% DEA); Mobile Phase B: Methanol; Flow rate: 20 mL/min; Gradient 1: 20% B isocratic 10 min; Gradient 2: 16% B isocratic in 8 min; Gradient 3: 25% B isocratic in 8 min; Gradient 4: 30% B isocratic in 7.5 min; Gradient 5: 40% B isocratic in 17 min; Gradient 6: 50% B isocratic in 7 min; Gradient 7: 40% B isocratic in 7 min; Gradient 8: 10% B in 17 min; Gradient 9: 35% B isocratic in 7.5 min; Gradient 10: 16% B isocratic in 9.5 min; Gradient 11: 20% B isocratic in 7 min; Gradient 12: 30% B isocratic in 6.5 min; Gradient 13: 25% B isocratic in 8.5 min; Gradient 14: 20% B isocratic in 9 min; Gradient 15: 50% B isocratic in 7.5 min.
Condition 5: Column: CHIRAL ART Cellulose-SB, 4.6×100 mm, 3 m; Mobile Phase A: MtBE(0.1% DEA): MeOH=60: 40; Flow rate: 1 mL/min; Gradient 1: 0% B to 0% B Condition 6: Column: CHIRALPAK IE, 2×25 cm, 5 m; Mobile Phase A: MtBE(0.1% DEA), Mobile Phase B: MeOH; Flow rate: 20 mL/min; Gradient 1: 45% B to 45% B in 14.5 min; Gradient 2: 20% B isocratic in 27 min.
Condition 7: Column: CHIRALPAK IG, 3×25 cm, 5 m; Mobile Phase A: Hex: DCM=1: 1, Mobile Phase B: EtOH (0.1% IPAmine); Flow rate: 35 mL/min; Gradient 1: 50% B isocratic in 40 min.
Condition 8: Column, CHIRALPAK IC, 2×25 cm, 5 μm; Mobile Phase A, Hex (0.1% DEA) Mobile Phase B:EtOH; Gradient 1: 30% B isocratic in 9 min; Gradient 2: 50% B isocratic in 18.5 min.
Condition 9: Column, YMC-Actus Triart C18, 30×150 mm, 5 μm; Mobile Phase A, water (10 mmol/L NH₄HCO₃), Mobile Phase B, ACN; Gradient 1: 20% B to 63% B in 8 min; Gradient 2: 15% B to 55% B in 8 min.
Condition 10: Column: CHIRAL ART Cellulose-SB, 2×25 cm, 5 μm; Mobile Phase A, MtBE (0.5% IPAmine), Mobile Phase B, MeOH; Gradient 1: 25% B isocratic 7 min.
Condition 11: Column: Xselect CSH C18 OBD, 30×150 mm, 5 m; Mobile Phase A, water (10 mmol/L NH₄HCO₃), Mobile Phase B, ACN; Gradient 1: 20% B to 80% B in 8 min.
Condition 12: Column: XBridge Prep OBD C18 Column, 30×150 mm, 5 μm; Mobile Phase A, water (10 mmol/L NH4HCO₃), and Mobile Phase B, ACN; Gradient 1: 20% ACN up to 70% in 8 min; Gradient 2: 34% B to 60% B in 8 min.
Condition 13: Column: CHIRAL ART Cellulose-SB, 2*25 cm, 5 m; Mobile Phase A, Hex(0.1% DEA), Mobile Phase B: EtOH; Gradient 1: 30% B isocratic 11 min.
Condition 14: Column: XBridge Shield RP18 OBD Column, 30*150 mm, 5 μm; Mobile phase A, water (10 mmol/L NH₄HCO₃), Mobile Phase B, ACN; Gradient 1: 10% B to 45% B in 10 min.
Condition 15: Column: YMC Triart C18 ExRS, 20*150 mm, 5 μm; Mobile phase A, water (10 mmol/L NH₄HCO₃), Mobile Phase B acetonitrile; Gradient 1: 20% B up to 63% B in 8 min.
Condition 16: Column: CHIRALPAK IF, 2*25 cm, 5 m; Mobile Phase A: MtBE(0.1% DEA), Mobile Phase B MeOH; Gradient 1: 15% B to 15% B in 19 min; Condition 17: Column: CHIRALPAK IG, 2*25 cm, 5 m; Mobile Phase A: MtBE(0.1% DEA), Mobile Phase B: MeOH; Gradient 1: 30% B to 30% B in 17 min; Condition 18: Column: CHIRAL ART Cellulose-SZ, 3*25 cm, 5 μm; Mobile Phase A: CO₂, Mobile Phase B: MEOH: DCM=2: 1(0.1% 2M NH₃-MeOH); Gradient 1: isocratic 45% B.
Condition 19: Column: XBridge Prep OBD C18 Column, 30*150 mm, 5 m; Mobile Phase A: Water (0.05% NH3·H2O), Mobile Phase B: ACN; Gradient 1: 10% B to 60% B in 8 min.
Condition 20: Column: CHIRALPAK IH, 3*25 cm, 5 μm; Mobile Phase A: CO2, Mobile Phase B: MEOH:DCM=2:1(0.1% 2M NH₃-MeOH); Flow rate: 80 mL/min; Gradient 1: isocratic 40% B.
Condition 21: Column: Kinetex EVO prep C18, 30*150, Sum; Mobile Phase A: water (10 mmol/L NH₄HCO₃), Mobile Phase B: CAN; Gradient 1: 25% B to 60% B in 8 min.

### General Schemes

Compounds of the present disclosure may be prepared using a synthetic protocol illustrated below in Schemes A, B, C, D, E, F, and G.

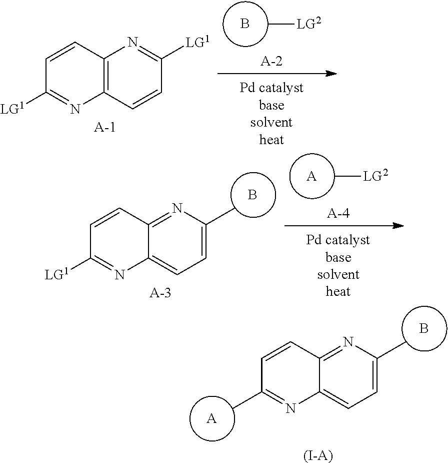

Scheme A. An exemplary method of preparing a representative compound of Formula (I-A); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

An exemplary method of preparing a compound of Formula (I-A) is provided in Scheme A. In this scheme, A-3 is prepared by incubating A-1 with A-2 in the presence of a base, for example, potassium carbonate (K₂CO₃) in dioxane and water or another suitable reagent. In some instances, A-3 is prepared by heating the reaction to a suitable temperature, for example, 80° C. Step 1 may also be carried out using an alternative catalyst to Pd₂(dba)₃, such as another palladium catalyst, for example, [1,1′-bis(di-tert-butylphosphino)ferrocene]dichloropalladium(II) (Pd(dtbpf)Cl₂) or chloro(2-dicyclohexylphosphino-2′,4′,6′-triisopropyl-1,1′-biphenyl)[2-(2′-amino-1,1′-biphenyl)]palladium(II) (XPhos-Pd-G2). The reaction may be conducted in DMF or a similar solvent, at 100° C. or a temperature sufficient to provide Fragment A-3, for example, 80° C., 90° C., 110° C., or 120° C. The reaction may be conducted in a microwave reactor.

In Step 2, A-3 and A-4 are coupled to provide a compound of Formula (I). This coupling reaction may be conducted in the presence of Pd(dppf)Cl₂, and K₂CO₃ or a similar reagent, for example tripotassium carbonate (K₃PO₄). As in Step 1, alternative catalysts to Pd(dppf)Cl₂ may be used, such as any suitable palladium catalyst, for example, tetrakis(triphenylphosphine)-palladium(0) (Pd(PPh₃)₄) or chloro(2-dicyclohexylphosphino-2′,4′,6′-triisopropyl-1,1′-biphenyl)[2-(2′-amino-1,1′-biphenyl)]palladium(II) (XPhos-Pd-G2). The reaction of Step 2 is conducted in dioxane or a mixture of dioxane and water, or other suitable solvents, and the mixture is heated to 80° C. or a temperature sufficient to provide the compound of Formula (I-A) or a precursor to the compound of Formula (I-A) with one or more protecting group(s), for example, 100° C. Compounds of Formula (I-A) may be purified using standard techniques and characterized using any method known in the art, such as nuclear magnetic resonance spectroscopy (NMR) or mass spectrometry (MS).

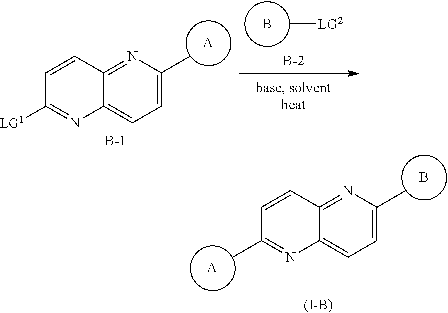

Scheme B. An exemplary method of preparing a representative compound of Formula (I-B); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

An exemplary method of preparing a compound of Formula (I-B) is provided in Scheme B. In this scheme, the compound of Formula (I-B) is prepared by incubating A-1 with A-2 in the presence of a base, for example, potassium carbonate (K₂CO₃) or another suitable reagent. In some instances, A-3 is prepared by heating the reaction to a suitable temperature, for example, 100° C. The reaction may be conducted in DMF or a similar solvent, at 100° C. or a temperature sufficient to provide the compound of Formula (I-B), for example, 80° C., 90° C., 110° C., or 120° C. to provide the compound of Formula (I-B) or a precursor to the compound of Formula (I-B) with one or more protecting group(s), for example, 100° C. Compounds of Formula (I-B) may be purified using standard techniques and characterized using any method known in the art, such as nuclear magnetic resonance spectroscopy (NMR) or mass spectrometry (MS). The reaction may be conducted in a microwave reactor.

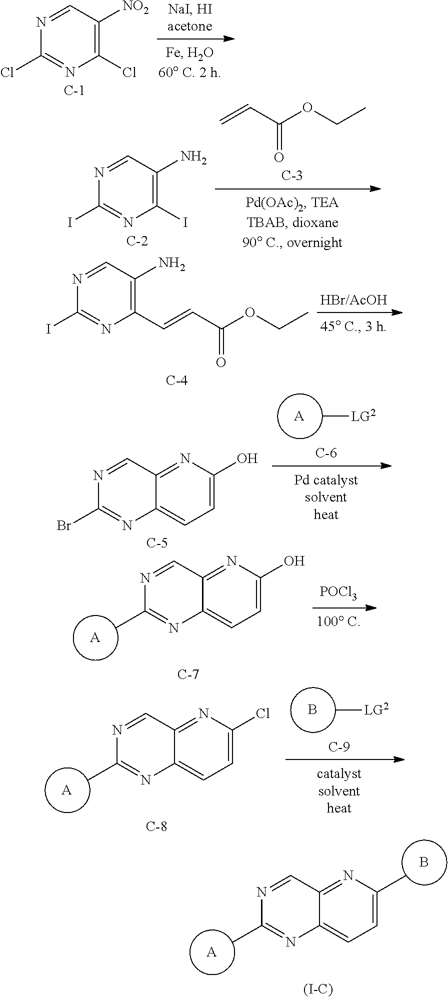

Scheme C. An exemplary method of preparing a representative compound of Formula (I-C); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

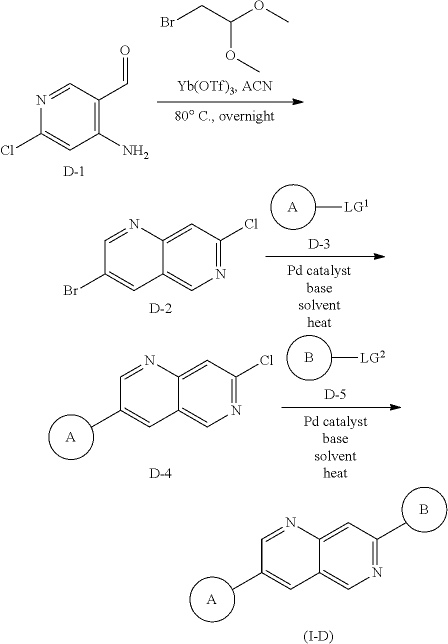

Scheme D. An exemplary method of preparing a representative compound of Formula (I-D); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

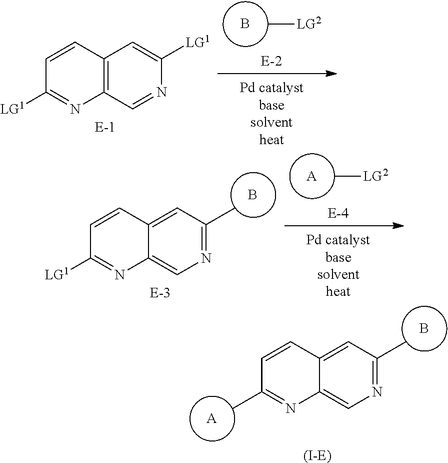

Scheme E. An exemplary method of preparing a representative compound of Formula (I-E); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

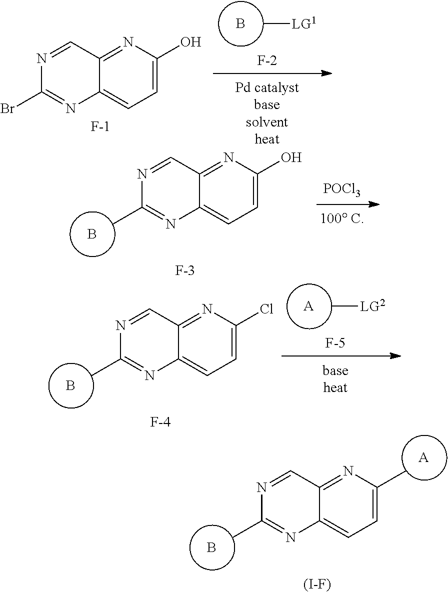

Scheme F. An exemplary method of preparing a representative compound of Formula (I-F); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

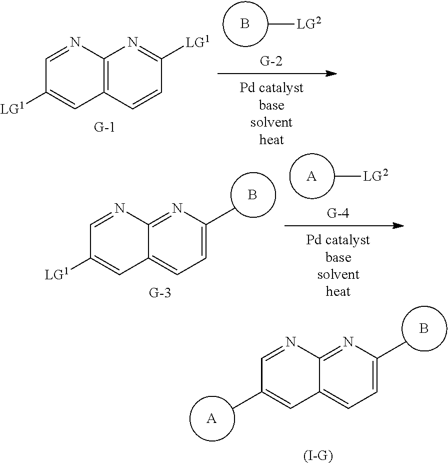

Scheme G. An exemplary method of preparing a representative compound of Formula (I-G); wherein A, and B are as defined herein, LG¹ and LG² are each independently a leaving group (e.g., halo); and —B(OR¹²)₂ is a boronic ester (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl.

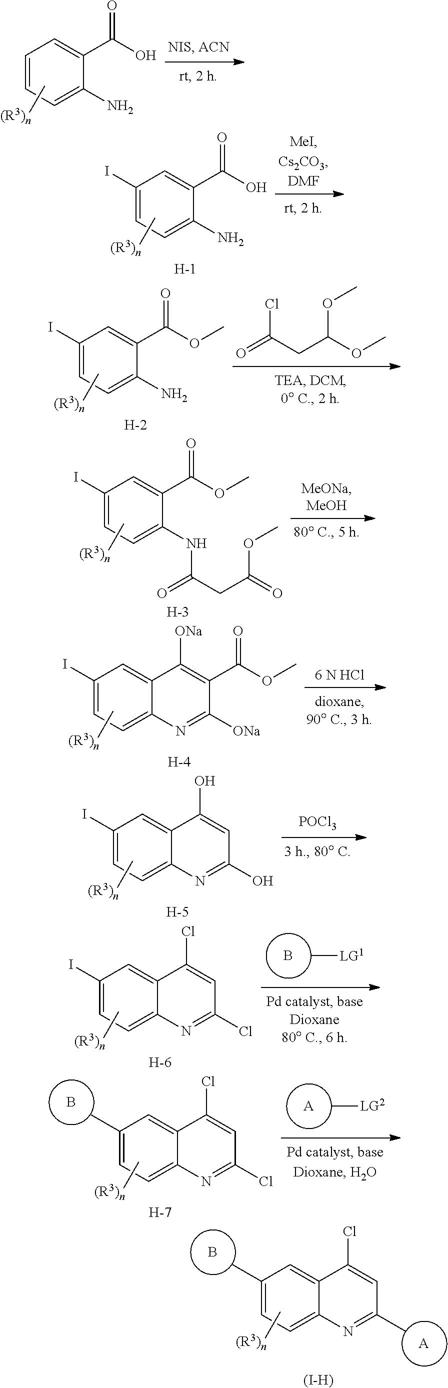

Scheme H. An exemplary method of preparing a representative compound of Formula (I-H); wherein A, B, R³, and n are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl or hydrogen.

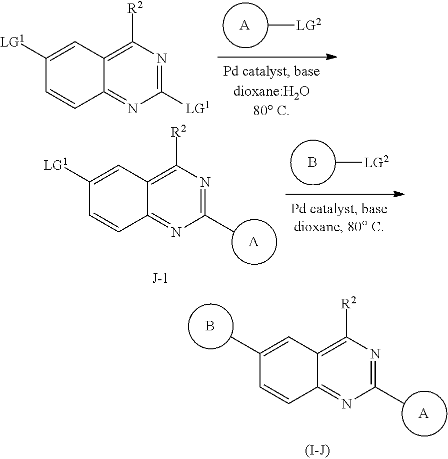

Scheme J. An exemplary method of preparing a representative compound of Formula (I-J); wherein A, B, and R² are as defined herein, each instance of LG¹ and LG² is each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl or hydrogen.

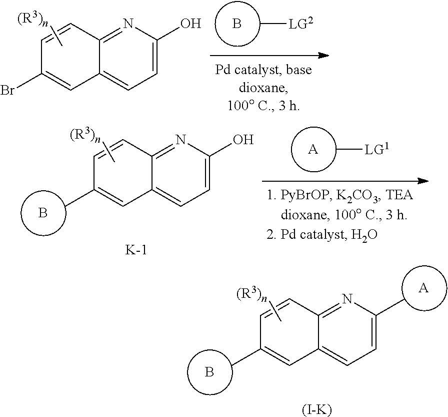

Scheme K. An exemplary method of preparing a representative compound of Formula (I-K); wherein A, B, R³, and n are as defined herein, LG¹ and LG² are each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl or hydrogen.

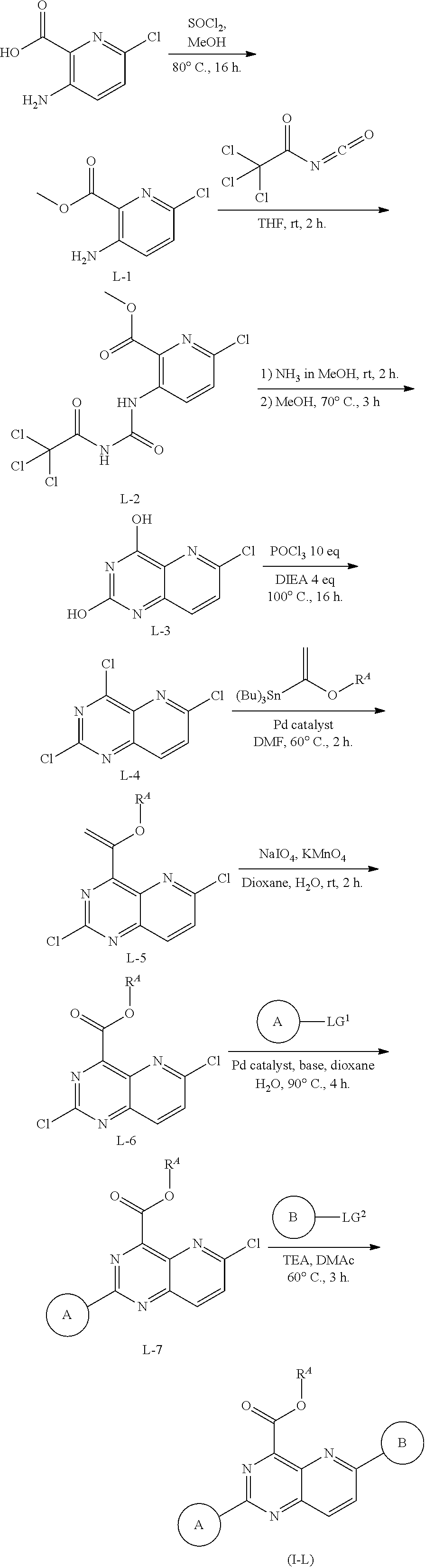

Scheme L. An exemplary method of preparing a representative compound of Formula (I-L); wherein A, B, and R^{A} are as defined herein, each instance of LG¹ and LG² is each independently a leaving group such as halo, a boronic ester —B(OR¹²)₂ (e.g., Bpin), wherein each R¹² may be C₁-C₆-alkyl, C₂-C₆-heteroalkyl, aryl, or heteroaryl; or two R¹² groups, together with the atoms to which they are attached, form a heterocyclyl or heteroaryl or hydrogen.

Exemplary methods of preparing a compound of Formula (I) or (II) are provided in Schemes A-G. Coupling of Ring A or Ring B to the core may be carried out with a catalyst, for example, a palladium catalyst, such as Pd₂(dba)₃, tetrakis(triphenylphosphine)-palladium(0) (Pd(PPh₃)₄), 1,1′-bis(diphenylphosphino)ferrocene)palladium(II) dichloride (Pd(dppf)Cl₂), [1,1′-bis(di-tert-butylphosphino)ferrocene]dichloropalladium(II) (Pd(dtbpf)Cl₂), Pd-PEPPSI-IPentCl 2-methylpyridine o-picoline, chloro(2-dicyclohexylphosphino-2′,4′,6′-triisopropyl-1,1′-biphenyl)[2-(2′-amino-1,1′-biphenyl)]palladium(II) (XPhos-Pd-G2), SPhos-Pd-G3, or XPhos-Pd-G3, and/or a copper catalyst, such as CuI or Cu(OAc)₂ in the presence or absence of a nitrogenous ligand. One or more bases, such as potassium carbonate, cesium carbonate, potassium phosphate, or triethyl amine, may also be present. Coupling reactions may be conducted in a solvent, such as DMA, DMF, DCM, THF, toluene, dioxane, water, or a similar solvent or mixtures of solvents, at room temperature or a temperature sufficient to provide the compound of Formula (I) or (II), for example, 80° C., 90° C., 100° C., 110° C., or 120° C. The reaction may be conducted in a microwave reactor. Compounds of Formula (I) or (II) may be purified using standard techniques and characterized using any method known in the art, such as nuclear magnetic resonance spectroscopy (NMR) or mass spectrometry (MS).

Examples 1-102 describing exemplary protocols for Compound 100-282 are provided in WO 2022/006550, which is incorporated herein by reference in its entirety.

### Example 103: Synthesis of Compound 343

### Synthesis of Intermediate C1

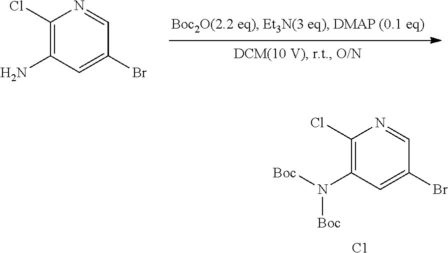

A mixture of 5-bromo-2-chloropyridin-3-amine (15.00 g, 72.30 mmol, 1 equiv), Boc₂O (34.70 g, 159.06 mmol, 2.2 equiv), Et₃N (21.90 g, 216.90 mmol, 3 equiv), and DMAP (0.86 g, 7.23 mmol, 0.1 equiv) in DCM (150 mL) was stirred overnight at room temperature. The resulting mixture was diluted with water (100 mL) and extracted with CH₂Cl₂ (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (3:2) to afford tert-butyl N-(5-bromo-2-chloropyridin-3-yl)-N-(tert-butoxycarbonyl)carbamate (21.90 g, 74%) as a solid. LCMS (ES, m/z): 407 [M+H]⁺.

### Synthesis of Intermediate C2

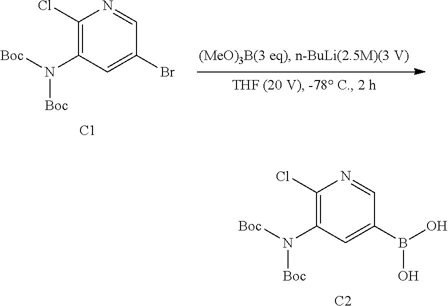

To a stirred mixture of tert-butyl N-(5-bromo-2-chloropyridin-3-yl)-N-(tert-butoxycarbonyl)carbamate (21.90 g, 53.71 mmol, 1 equiv) and B(OMe)₃ (16.70 g, 161.15 mmol, 3 equiv) in THE (440 mL) was added n-BuLi (64.6 mL) dropwise at −78° C. under nitrogen atmosphere. The resulting mixture was stirred for 2 h at −78° C. under nitrogen atmosphere. The reaction mixture was quenched with sat. NH₄Cl (aq.) at 0° C., then acidified to pH 3 with 2 M HCl (aq.) and extracted with ethyl acetate (3×1000 mL). The organic layers were combined, washed with brine (1×1000 mL), dried over anhydrous Na₂SO₄, and filtered. After filtration, the filtrate was concentrated under reduced pressure to afford 5-[bis(tert-butoxycarbonyl)amino]-6-chloropyridin-3-ylboronic acid (24.3 g, 100%) as an oil. LCMS (ES, m/z): 373 [M+H]⁺.

### Synthesis of Intermediate C3

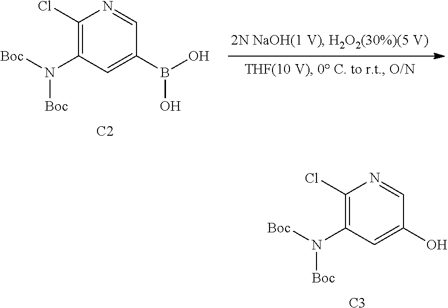

To a stirred mixture of 5-[bis(tert-butoxycarbonyl)amino]-6-chloropyridin-3-ylboronic acid (24.30 g, 65.21 mmol, 1 equiv) and 2 M NaOH (24 mL) in THE (240 mL) was added H₂02 (30%) (120 mL) dropwise at 0° C. The resulting mixture was stirred for 3 hr at room temperature, then quenched with sat. NaHSO₃ (aq.) at 0° C. and extracted with ethyl acetate (3×300 mL). The organic layers were combined, washed with brine (1×300 mL), dried over anhydrous Na₂SO₄, and filtered. After filtration, the filtrate was concentrated under reduced pressure to afford tert-butyl N-(tert-butoxycarbonyl)-N-(2-chloro-5-hydroxypyridin-3-yl)carbamate (19.00 g, 85%) as an oil. LCMS (ES, m/z): 345 [M+H]⁺.

### Synthesis of Intermediate C4

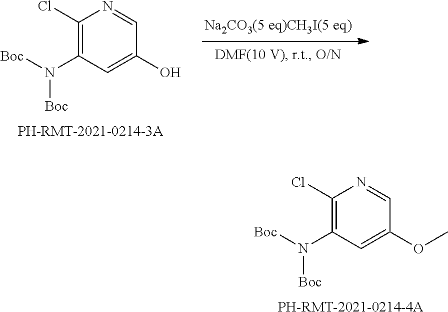

To a stirred mixture of tert-butyl N-(tert-butoxycarbonyl)-N-(2-chloro-5-hydroxypyridin-3-yl)carbamate (19.00 g, 55.10 mmol, 1 equiv) and Na₂CO₃ (29.20 g, 275.53 mmol, 5 equiv) in DMF (200 mL) was added CH₃I (39.10 g, 275.53 mmol, 5 equiv) dropwise at 0° C. under nitrogen atmosphere. The resulting mixture was stirred overnight at room temperature under nitrogen atmosphere, then diluted with water (200 mL) and extracted with ethyl acetate (3×200 mL). The organic layers were combined, washed with brine (1×200 mL), dried over anhydrous Na₂SO₄, and filtered. After filtration, the filtrate was concentrated under reduced pressure to give a residue. The residue was purified by silica gel column chromatography, eluted with PE/EA (3:2) to afford tert-butyl N-(tert-butoxycarbonyl)-N-(2-chloro-5-methoxypyridin-3-yl)carbamate (10.2 g, 52%) as a solid. LCMS (ES, m/z): 359 [M+H]⁺.

### Synthesis of Intermediate C5

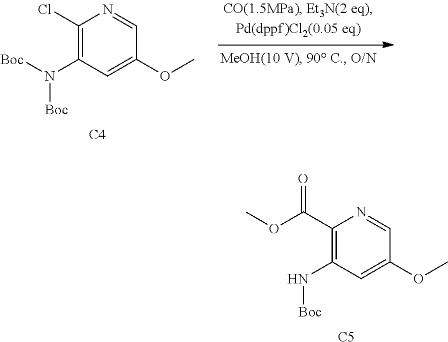

To a solution of tert-butyl N-(tert-butoxycarbonyl)-N-(2-chloro-5-methoxypyridin-3-yl)carbamate (10.20 g, 28.42 mmol, 1 equiv) in methanol (100 mL) was added Et₃N (14.40 g, 142.13 mmol, 5 equiv) and Pd(dppf)Cl₂CH₂Cl₂ (1.20 g, 1.42 mmol, 0.05 equiv) in a pressure tank. The mixture was purged with nitrogen for 30 min, then was pressurized to 1.5 MPa with carbon monoxide and heated at 90° C. overnight. The reaction mixture was cooled to room temperature. A precipitate formed that was collected to afford methyl 3-[(tert-butoxycarbonyl)amino]-5-methoxypyridine-2-carboxylate (4.40 g, 55%) as a solid. LCMS (ES, m/z): 283 [M+H]⁺.

### Synthesis of Intermediate C6

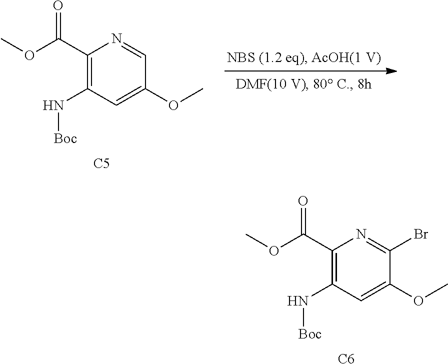

A mixture of methyl 3-[(tert-butoxycarbonyl)amino]-5-methoxypyridine-2-carboxylate (4.40 g, 15.58 mmol, 1 equiv), NBS (3.10 g, 17.14 mmol, 1.1 equiv), and AcOH (4.4 mL) in DMF (44 mL) was stirred overnight at 60° C. The resulting mixture was diluted with water (100 mL) and extracted with ethyl acetate (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (3:2) to afford methyl 6-bromo-3-[(tert-butoxycarbonyl)amino]-5-methoxypyridine-2-carboxylate (3.37 g, 60%) as a solid. LCMS (ES, m/z): 361 [M+H]+.

### Synthesis of Intermediate C7

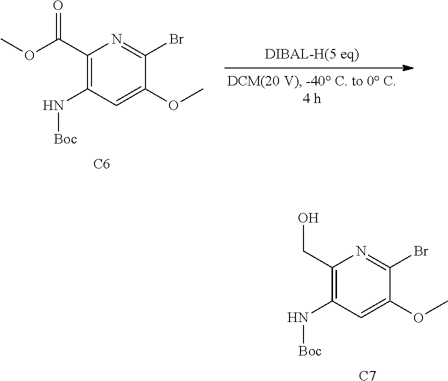

To a stirred solution of methyl 6-bromo-3-[(tert-butoxycarbonyl)amino]-5-methoxypyridine-2-carboxylate (3.37 g, 9.33 mmol, 1 equiv) in DCM (70 mL) was added DIBAl-H (7.96 g, 55.98 mmol, 6 equiv) dropwise at −40° C. under nitrogen atmosphere. The resulting mixture was stirred for 3 h at 0° C. under nitrogen atmosphere. The resulting mixture was diluted with water (100 mL), neutralized to pH 8 with 2 M NaOH, and extracted with CH₂Cl₂ (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (20:1) to afford tert-butyl N-[6-bromo-2-(hydroxymethyl)-5-methoxypyridin-3-yl]carbamate (1.84 g, 59%) as a solid. LCMS (ES, m/z): 333 [M+H]⁺.

### Synthesis of Intermediate C8

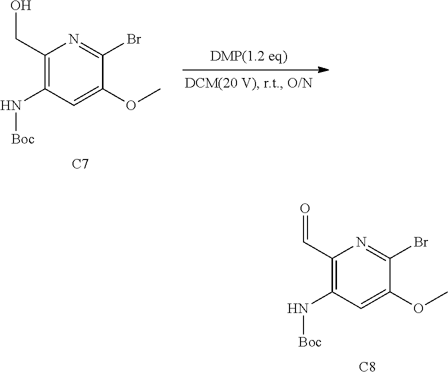

A mixture of tert-butyl N-[6-bromo-2-(hydroxymethyl)-5-methoxypyridin-3-yl]carbamate (1.84 g, 5.52 mmol, 1 equiv) and DMP (2.81 g, 6.62 mmol, 1.2 equiv) in DCM (20 mL) was stirred overnight at room temperature under nitrogen atmosphere. The resulting mixture was concentrated under vacuum to give a residue. The residue was purified by silica gel column chromatography, eluted with PE/EA (20:1) to afford tert-butyl N-(6-bromo-2-formyl-5-methoxypyridin-3-yl)carbamate (1.60 g, 88%) as a solid. LCMS (ES, m/z): 331 [M+H]⁺.

### Synthesis of Intermediate C9

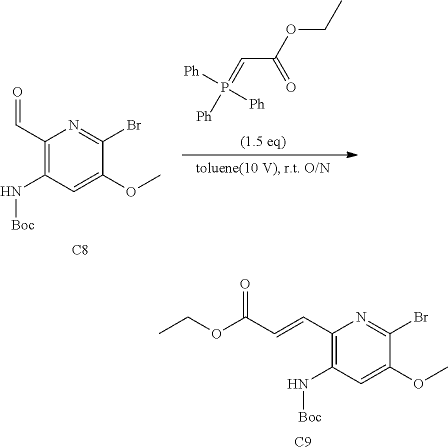 [SMILES: COc1cc(NC(=O)OC(C)(C)C)c(C=O)nc1Br ; CCOC(=O)C=P(c1ccccc1)(c1ccccc1)c1ccccc1 ; "C8" ; CCOC(=O)/C=C/c1nc(Br)c(OC)cc1NC(=O)OC(C)(C)C ; "C9"]

A mixture of tert-butyl N-(6-bromo-2-formyl-5-methoxypyridin-3-yl)carbamate (1.60 g, 4.83 mmol, 1 equiv) and ethyl 2-(triphenyl-lambda5-phosphanylidene)acetate (2.19 g, 6.28 mmol, 1.3 equiv) in toluene (16 mL) was stirred for 5 h at room temperature. The resulting mixture was concentrated under vacuum to give a residue. The residue was purified by silica gel column chromatography, eluted with PE/EA (5:1) to afford ethyl (2E)-3-{6-bromo-3-[(tert-butoxycarbonyl)amino]-5-methoxypyridin-2-yl}prop-2-enoate (1.3 g, 67%) as a solid. LCMS (ES, m/z): 401 [M+H]⁺.

### Synthesis of Intermediate C10

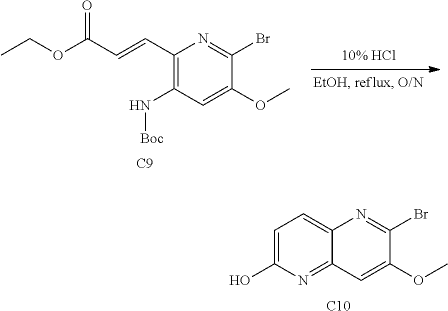 [SMILES: CCOC(=O)/C=C/c1nc(Br)c(OC)cc1NC(=O)OC(C)(C)C ; "C9" ; COc1cc2nc(O)ccc2nc1Br ; "C10"]

A solution of ethyl (2E)-3-{6-bromo-3-[(tert-butoxycarbonyl)amino]-5-methoxypyridin-2-yl}prop-2-enoate (1.30 g, 3.24 mmol, 1 equiv) in 10% HCl (26 mL) and ethanol (6.5 mL) was stirred overnight at 100° C. The resulting mixture was filtered, and the filter cake washed with water (3×20 mL), followed by PE (3×20 mL). The filtrate was concentrated under reduced pressure to afford 6-bromo-7-methoxy-1,5-naphthyridin-2-ol (600 mg, 73%) as a solid. LCMS (ES, m/z): 255 [M+H]⁺.

### Synthesis of Intermediate C11

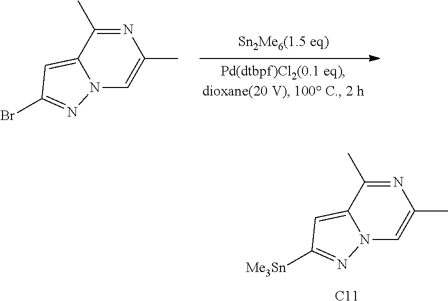

A mixture of 2-bromo-4,6-dimethylpyrazolo[1,5-a]pyrazine (500 mg, 2.21 mmol, 1 equiv), Sn₂Me₆ (1086.9 mg, 3.31 mmol, 1.5 equiv), and Pd(DtBPF)Cl₂ (144.1 mg, 0.22 mmol, 0.1 equiv) in dioxane (10 mL) was stirred for 2 hr at 100° C. under nitrogen atmosphere. The reaction mixture was quenched with sat. KF (aq.)(30 mL) at room temperature and extracted with CH₂Cl₂ (3×30 mL). The organic layers were combined, washed with brine (1×30 mL), dried over anhydrous Na₂SO₄, and filtered. After filtration, the filtrate was concentrated under reduced pressure to afford 4,6-dimethyl-2-(trimethylstannyl)pyrazolo[1,5-a]pyrazine (680 mg, Crude) as an oil. LCMS (ES, m/z): 312 [M+H]⁺.

### Synthesis of Intermediate C12

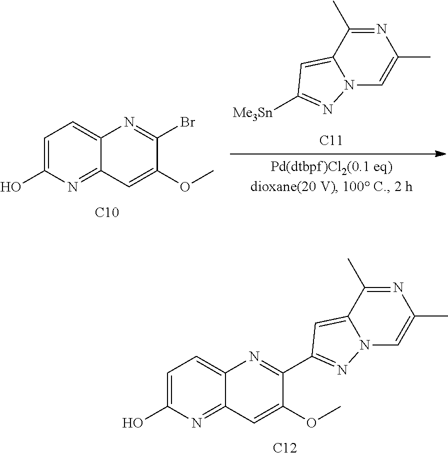

A mixture of 6-bromo-7-methoxy-1,5-naphthyridin-2-ol (300 mg, 1.17 mmol, 1 equiv), 4,6-dimethyl-2-(trimethylstannyl)pyrazolo[1,5-a]pyrazine (401.0 mg, 1.29 mmol, 1.1 equiv), and Pd(DtBPF)Cl₂ (76.6 mg, 0.12 mmol, 0.1 equiv) in dioxane (3 mL) was stirred for 2 hr at 100° C. under nitrogen atmosphere. The resulting mixture was concentrated under vacuum to give a residue. The residue was purified by silica gel column chromatography, eluted with CH₂Cl₂/MeOH (5:1) to afford 6-{4,6-dimethylpyrazolo[1,5-a]pyrazin-2-yl}-7-methoxy-1,5-naphthyridin-2-ol (240 mg, 64%) as a solid. LCMS (ES, m/z): 322 [M+H]⁺.

### Synthesis of Intermediate C13

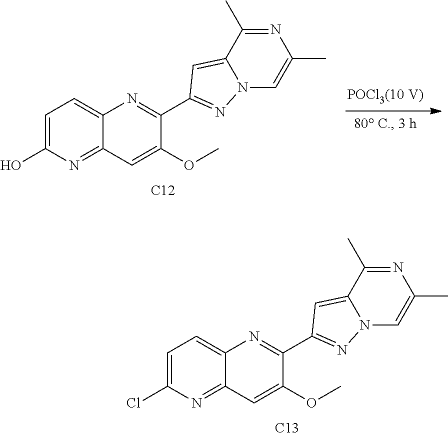 [SMILES: COc1cc2nc(O)ccc2nc1-c1cc2c(C)nc(C)cn2n1 ; "C12" ; COc1cc2nc(Cl)ccc2nc1-c1cc2c(C)nc(C)cn2n1 ; "C13"]

A solution of 6-{4,6-dimethylpyrazolo[1,5-a]pyrazin-2-yl}-7-methoxy-1,5-naphthyridin-2-ol (230 mg, 0.71 mmol, 1 equiv) in POCl₃ (3 mL) was stirred for 3 hr at 80° C. The resulting mixture was concentrated under vacuum to give a residue. The residue was purified by silica gel column chromatography, eluted with CH₂Cl₂/MeOH (5:1) to afford 6-chloro-2-{4,6-dimethylpyrazolo[1,5-a]pyrazin-2-yl}-3-methoxy-1,5-naphthyridine (300 mg, 100%) as an oil. LCMS (ES, m/z): 340 [M+H]⁺.

### Synthesis of Intermediate C14

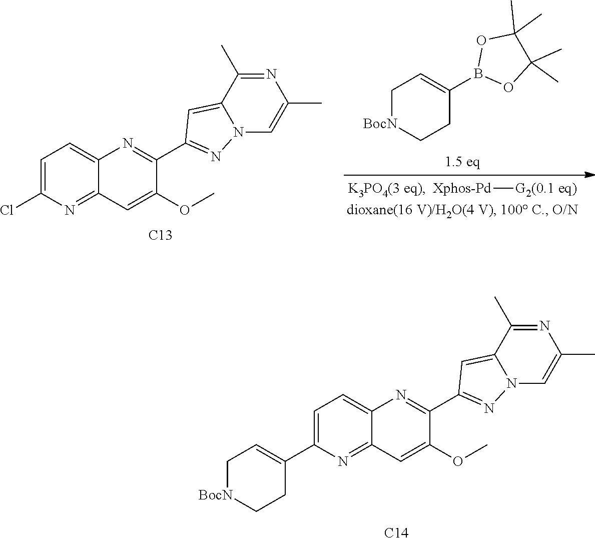

A mixture of 6-chloro-2-{4,6-dimethylpyrazolo[1,5-a]pyrazin-2-yl}-3-methoxy-1,5-naphthyridine (255 mg, 0.75 mmol, 1 equiv), tert-butyl 4-(4,4,5,5-tetramethyl-1,3,2-dioxaborolan-2-yl)-3,6-dihydro-2H-pyridine-1-carboxylate (348.1 mg, 1.12 mmol, 1.5 equiv), K₃PO₄ (477.9 mg, 2.25 mmol, 3 equiv) and XPhos Precatalyst (2nd Generation, 59.0 mg, 0.07 mmol, 0.1 equiv) in dioxane (2 mL) and water (0.5 mL) was stirred overnight at 100° C. under nitrogen atmosphere. The resulting mixture was diluted with water (20 mL) and extracted with ethyl acetate (3×20 mL). The organic layers were combined, washed with brine (1×20 mL), dried over anhydrous Na₂SO₄, and filtered. After filtration, the filtrate was concentrated under reduced pressure to give a residue. The residue was purified by silica gel column chromatography, eluted with PE/EA (3:2) to afford tert-butyl 4-(6-{4,6-dimethylpyrazolo[1,5-a]pyrazin-2-yl}-7-methoxy-1,5-naphthyridin-2-yl)-3,6-dihydro-2H-pyridine-1-carboxylate (60 mg) as a solid. LCMS (ES, m/z): 487 [M+H]⁺.

### Synthesis of Intermediate C15

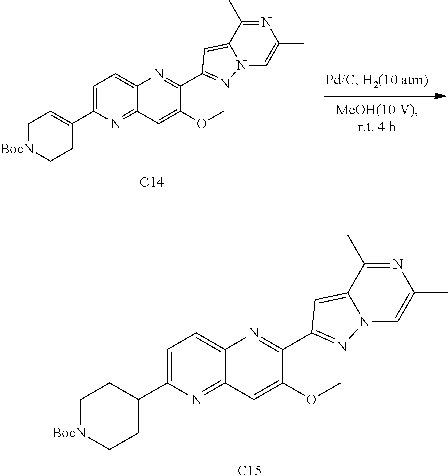 [SMILES: COc1cc2nc(C3=CCN(C(=O)OC(C)(C)C)CC3)ccc2nc1-c1cc2c(C)nc(C)cn2n1 ; "C14" ; COc1cc2nc(C3CCN(C(=O)OC(C)(C)C)CC3)ccc2nc1-c1cc2c(C)nc(C)cn2n1 ; "C15"]

To a solution of tert-butyl 4-(6-{4,6-dimethylpyrazolo[1,5-a]pyrazin-2-yl}-7-methoxy-1,5-naphthyridin-2-yl)-3,6-dihydro-2H-pyridine-1-carboxylate (60 mg, 0.12 mmol, 1 equiv) in 2 mL MeOH was added Pd/C (10%, 120 mg) in a pressure tank. The mixture was hydrogenated at room temperature under 10 psi of hydrogen pressure for 4 h, filtered through a Celite pad, and concentrated under reduced pressure to afford tert-butyl 4-(6-{4,6-dimethylpyrazolo[1,5-a]pyrazin-2-yl}-7-methoxy-1,5-naphthyridin-2-yl)piperidine-1-carboxylate (30 mg) as a solid. LCMS (ES, m/z): 489 [M+H]⁺.

### Synthesis of Compound 242

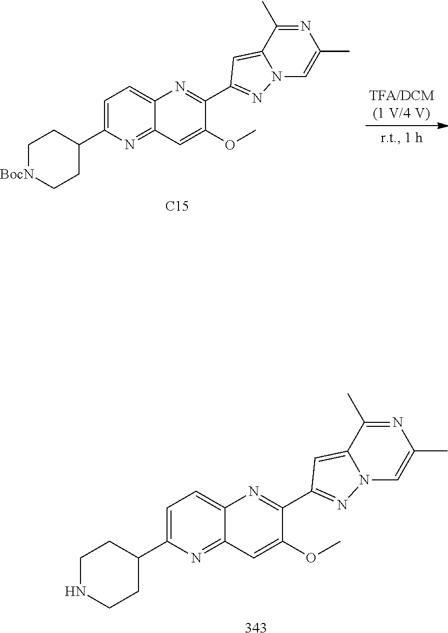 [SMILES: COc1cc2nc(C3CCN(C(=O)OC(C)(C)C)CC3)ccc2nc1-c1cc2c(C)nc(C)cn2n1 ; "C15" ; COc1cc2nc(C3CCNCC3)ccc2nc1-c1cc2c(C)nc(C)cn2n1 ; "343"]

A solution of tert-butyl 4-(6-{4,6-dimethylpyrazolo[1,5-a]pyrazin-2-yl}-7-methoxy-1,5-naphthyridin-2-yl)piperidine-1-carboxylate (14 mg, 0.029 mmol, 1 equiv) in DCM (2 mL) and TFA (0.5 mL) was stirred for 2 h at room temperature. The resulting mixture was concentrated under vacuum to give a residue. The residue was purified by Prep-HPLC (Condition 9, Gradient 1) to afford 2-{4,6-dimethylpyrazolo[1,5-a]pyrazin-2-yl}-3-methoxy-6-(piperidin-4-yl)-1,5-naphthyridine (1.5 mg, 13%) as a solid. LCMS (ES, m/z): 389 [M+H]⁺. ¹H NMR (400 MHz, DMSO-d₆) δ 8.57 (s, 1H), 8.35 (d, J=8.6 Hz, 1H), 7.90 (s, 1H), 7.73 (t, J=5.7 Hz, 0.2H), 7.62-7.53 (m, 2H), 4.11 (s, 3H), 3.13 (d, J=11.9 Hz, 2H), 3.09-2.94 (m, 1H), 2.75 (s, 3H), 2.70 (s, 1H), 2.67 (p, J=1.8 Hz, 1H), 2.46-2.39 (m, 3H), 2.02 (t, J=7.3 Hz, 0.4H), 1.90 (d, J=12.7 Hz, 2H), 1.78 (q, J=12.3 Hz, 2H), 1.46 (q, J=7.5 Hz, 0.5H), 1.35 (q, J=7.2 Hz, 0.5H), 1.24 (s, 1.6H), 0.83 (dd, J=10.6, 6.9 Hz, 0.7H).

### Example 104: Synthesis of Compounds 299 and 300

### Synthesis of Intermediate C16

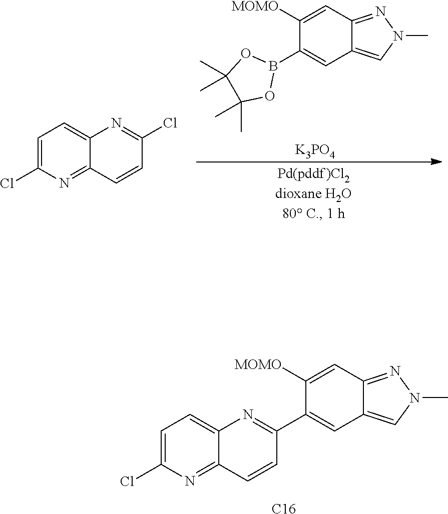

To a stirred mixture of 2,6-dichloro-1,5-naphthyridine (300 mg, 1.507 mmol, 1 equiv) and 6-(methoxymethoxy)-2-methyl-5-(4,4,5,5-tetramethyl-1,3,2-dioxaborolan-2-yl)indazole (479.60 mg, 1.507 mmol, 1 equiv) in dioxane (6 mL) and water (1.2 mL) was added Pd(dppf)Cl₂·CH₂Cl₂ (122.79 mg, 0.151 mmol, 0.1 equiv) and K₃PO₄ (959.85 mg, 4.521 mmol, 3 equiv) at room temperature under nitrogen atmosphere. The reaction mixture was stirred at 80° C. for 1 hr. The resulting mixture was concentrated under vacuum to give a residue. The residue was purified by silica gel column chromatography, eluted with PE/EA (1:1) to afford 2-chloro-6-[6-(methoxymethoxy)-2-methylindazol-5-yl]-1,5-naphthyridine (260 mg, 49%) as a solid.

### Synthesis of Intermediate C17

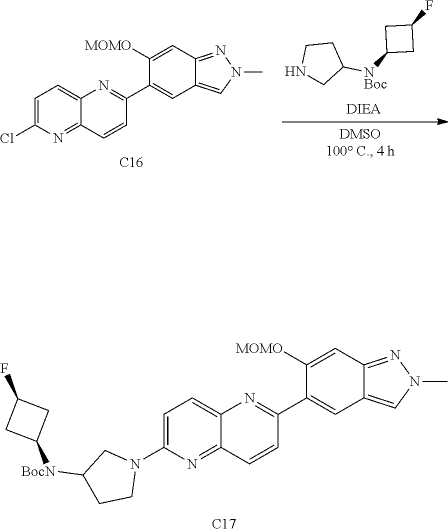

A mixture of 2-chloro-6-[6-(methoxymethoxy)-2-methylindazol-5-yl]-1,5-naphthyridine (190 mg, 0.536 mmol, 1 equiv) and tert-butyl N-(pyrrolidin-3-yl)-N-[(1s,3s)-3-fluorocyclobutyl]carbamate (166.02 mg, 0.643 mmol, 1.2 equiv) in DMSO (3.8 mL) was treated with DIEA (207.65 mg, 1.608 mmol, 3 equiv). The reaction mixture was stirred for 4 hr at 100° C., then cooled to room temperature. The resulting mixture was concentrated under vacuum to give a residue. The residue was purified by silica gel column chromatography, eluted with PE/EA (1:1) to afford tert-butyl N-(1-{6-[6-(methoxymethoxy)-2-methylindazol-5-yl]-1,5-naphthyridin-2-yl}pyrrolidin-3-yl)-N-[(1s,3s)-3-fluorocyclobutyl]carbamate (90 mg, 29%) as a solid.

### Synthesis of Compounds 299 and 300

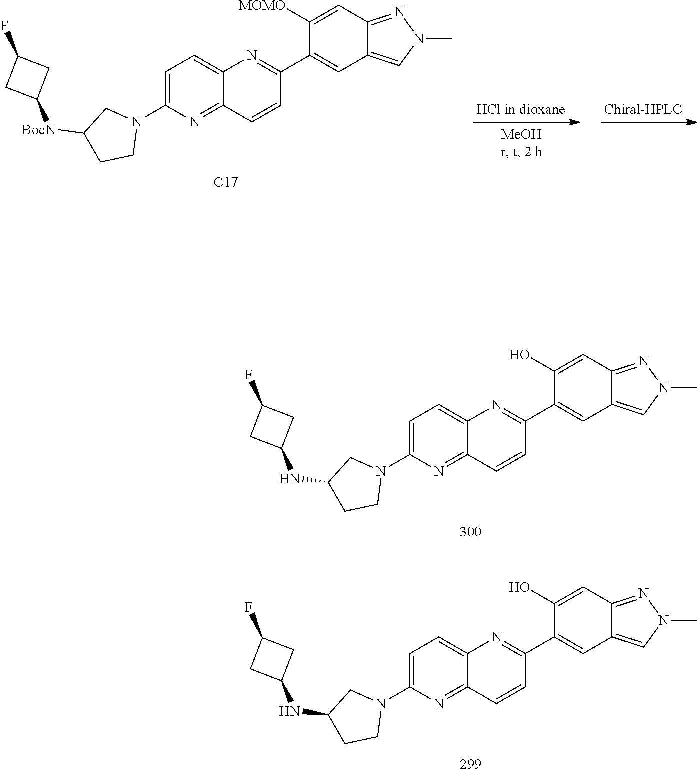

A mixture of tert-butyl N-(1-{6-[6-(methoxymethoxy)-2-methylindazol-5-yl]-1,5-naphthyridin-2-yl}pyrrolidin-3-yl)-N-[(1s,3s)-3-fluorocyclobutyl]carbamate (90 mg, 0.156 mmol, 1 equiv) and HCl (gas) in 1,4-dioxane (1 mL) in methanol (1 mL) was stirred for 2 h at room temperature under nitrogen atmosphere. The resulting mixture was concentrated under reduced pressure to give a residue. The residue was purified by Prep-Chiral--HPLC (Condition 4, Gradient 1) to afford 2-methyl-5-{6-[(3S)-3-{[(1s,3s)-3-fluorocyclobutyl]amino}pyrrolidin-1-yl]-1,5-naphthyridin-2-yl}indazol-6-ol (14.3 mg, 21%) as a solid. LCMS (ES, m/z): 433 [M+H]⁺. ¹H NMR (400 MHz, DMSO-d₆) δ 13.97 (s, 1H), 8.54 (s, 1H), 8.39 (d, J=9.1 Hz, 1H), 8.35 (s, 1H), 8.08 (dd, J=15.9, 9.2 Hz, 2H), 7.14 (d, J=9.3 Hz, 1H), 6.87 (s, 1H), 4.78 (dt, J=56.3, 6.9 Hz, 1H), 4.11 (s, 3H), 3.39 (s, 2H), 3.35 (s, 3H), 2.82-2.74 (m, 1H), 2.65 (dq, J=11.6, 5.7, 5.2 Hz, 2H), 2.10 (dq, J=12.8, 6.4 Hz, 1H), 1.98-1.75 (m, 3H).

### Example 105: Synthesis of Compound 345

### Synthesis of Intermediate C18

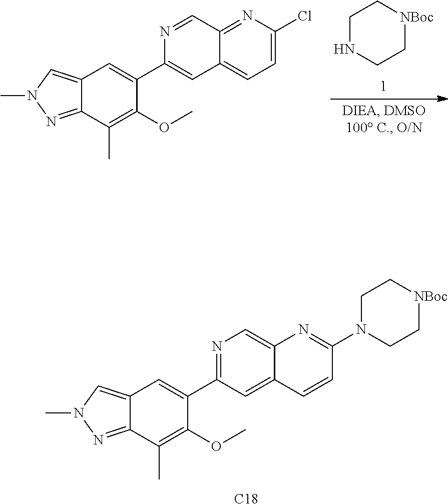 [SMILES: COc1c(-c2cc3ccc(Cl)nc3cn2)cc2cn(C)nc2c1C ; CC(C)(C)OC(=O)N1CCNCC1 ; COc1c(-c2cc3ccc(N4CCN(C(=O)OC(C)(C)C)CC4)nc3cn2)cc2cn(C)nc2c1C ; "C18"]

To a stirred mixture of 2-chloro-6-(6-methoxy-2,7-dimethylindazol-5-yl)-1,7-naphthyridine (57 mg, 0.16 mmol, 1 equiv) and tert-butyl piperazine-1-carboxylate (47 mg, 0.25 mmol, 1.5 equiv) in DMSO (1 mL) was added DIEA (65.23 mg, 0.50 mmol, 3 equiv) dropwise at room temperature under nitrogen atmosphere. The resulting mixture was stirred overnight at 100° C. The reaction mixture was quenched with water at room temperature and extracted with ethyl acetate (2×5 mL). The organic layers were combined, washed with brine (2×5 mL), dried over anhydrous Na₂SO₄, and filtered. After filtration, the filtrate was concentrated under reduced pressure to afford tert-butyl 4-[6-(6-methoxy-2,7-dimethylindazol-5-yl)-1,7-naphthyridin-2-yl]piperazine-1-carboxylate (20 mg, 24%) as a solid.

### Synthesis of Compound 345

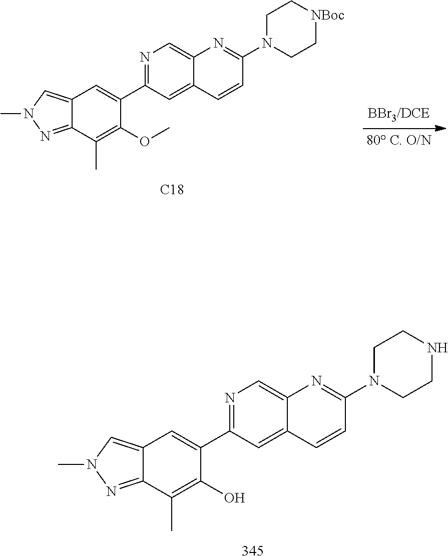 [SMILES: COc1c(-c2cc3ccc(N4CCN(C(=O)OC(C)(C)C)CC4)nc3cn2)cc2cn(C)nc2c1C ; "C18" ; Cc1c(O)c(-c2cc3ccc(N4CCNCC4)nc3cn2)cc2cn(C)nc12 ; "345"]

To a stirred mixture of tert-butyl 4-[6-(6-methoxy-2,7-dimethylindazol-5-yl)-1,7-naphthyridin-2-yl]piperazine-1-carboxylate (20 mg, 0.04 mmol, 1 equiv) in DCE (1 mL) was added BBr₃ (0.5 mL) dropwise at room temperature under nitrogen atmosphere. The resulting mixture was stirred overnight at 80° C. The reaction mixture was quenched with methanol at 0° C. The resulting mixture was concentrated under reduced pressure to give a residue. The residue was purified by Chiral-Prep-HPLC (Condition 10, Gradient 1) to afford 2,7-dimethyl-5-[2-(piperazin-1-yl)-1,7-naphthyridin-6-yl]indazol-6-ol (1.3 mg, 8%) as a solid. LCMS (ES, m/z): 375 [M+H]⁺. ¹H NMR (400 MHz, DMSO-d₆) δ 9.15 (s, 2H), 9.01 (s, 1H), 8.56 (d, J=0.9 Hz, 1H), 8.35 (s, 1H), 8.30-8.22 (m, 2H), 7.68 (d, J=9.4 Hz, 1H), 4.14 (s, 3H), 4.02 (t, J=5.3 Hz, 4H), 3.26 (s, 4H), 2.37 (s, 3H).

### Example 106: Synthesis of Compound 301

### Synthesis of Intermediate C19

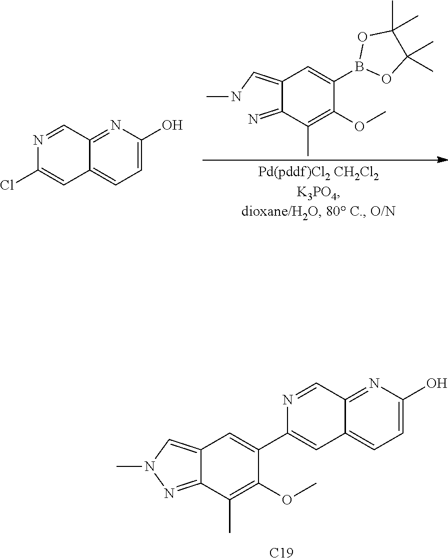

To a stirred mixture of 6-chloro-1,7-naphthyridin-2-ol (500 mg, 2.76 mmol, 1 equiv) and 6-methoxy-2,7-dimethyl-5-(4,4,5,5-tetramethyl-1,3,2-dioxaborolan-2-yl)indazole (1003.98 mg, 3.32 mmol, 1.2 equiv) in dioxane (5 mL) was added Pd(dppf)Cl₂CH₂Cl₂ (225.54 mg, 0.27 mmol, 0.1 equiv), K₃PO₄ (1763.10 mg, 8.307 mmol, 3 equiv), and water (1 mL) at room temperature under nitrogen atmosphere. The resulting mixture was stirred overnight at 80° C., then concentrated under reduced pressure to give a residue. The residue was purified by silica gel column chromatography, eluted with CH₂Cl₂/MeOH (10:1) to afford 6-(6-methoxy-2,7-dimethylindazol-5-yl)-1,7-naphthyridin-2-ol (500 mg, 39%) as a solid.

### Synthesis of Intermediate C20

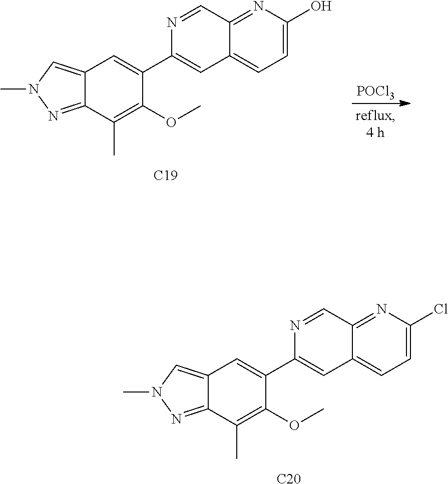

To mixture of 6-(6-methoxy-2,7-dimethylindazol-5-yl)-1,7-naphthyridin-2-ol (500 mg, 1.56 mmol, 1 equiv) in phosphorus oxychloride (10 mL) was stirred for 4 h at 80° C. The resulting mixture was concentrated under vacuum to afford 2-chloro-6-(6-methoxy-2,7-dimethylindazol-5-yl)-1,7-naphthyridine (550 mg, 73%) as a solid.

### Synthesis of Intermediate C21

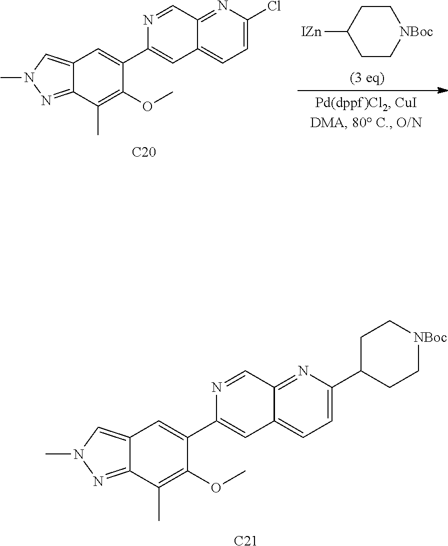

To a stirred mixture of 2-chloro-6-(6-methoxy-2,7-dimethylindazol-5-yl)-1,7-naphthyridine (250 mg, 0.73 mmol, 1 equiv), Pd(dppf)Cl₂·CH₂Cl₂ (60.11 mg, 0.07 mmol, 0.1 equiv), and CuI (28.11 mg, 0.14 mmol, 0.2 equiv) in DMA (3 mL) was added [1-(tert-butoxycarbonyl)piperidin-4-yl](iodo)zinc (555.70 mg, 1.47 mmol, 2 equiv) in portions at room temperature under nitrogen atmosphere. The resulting mixture was stirred overnight at 80° C., then quenched with water at room temperature. The resulting mixture was extracted with ethyl acetate (2×5 mL). The combined organic layers were washed with brine (2×10 mL), dried over anhydrous Na₂SO₄, and filtered. After filtration, the filtrate was concentrated under reduced pressure to afford tert-butyl 4-[6-(6-methoxy-2,7-dimethylindazol-5-yl)-1,7-naphthyridin-2-yl]piperidine-1-carboxylate (100 mg, 28%) as a solid.

### Synthesis of Compound 301

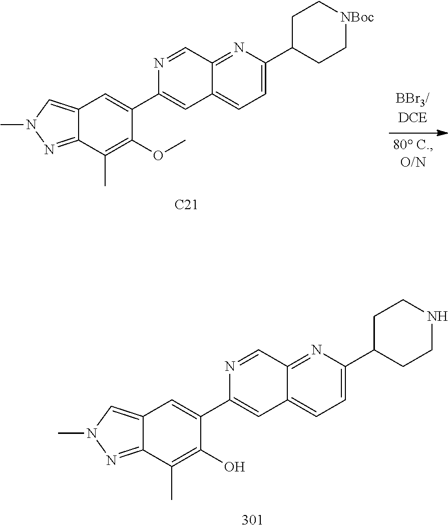 [SMILES: COc1c(-c2cc3ccc(C4CCN(C(=O)OC(C)(C)C)CC4)nc3cn2)cc2cn(C)nc2c1C ; "C21" ; Cc1c(O)c(-c2cc3ccc(C4CCNCC4)nc3cn2)cc2cn(C)nc12 ; "301"]

To a stirred solution of tert-butyl 4-[6-(6-methoxy-2,7-dimethylindazol-5-yl)-1,7-naphthyridin-2-yl]piperidine-1-carboxylate (30 mg, 0.06 mmol, 1 equiv) in DCE (1 mL, 0.01 mmol, 0.16 equiv) was added BBr₃ (1 mL, 0.004 mmol, 0.06 equiv) in portions at room temperature under nitrogen atmosphere. The resulting mixture was stirred overnight at 80° C., then quenched with methanol at 0° C., and filtered. After filtration, the filtrate was concentrated under reduced pressure to give a residue. The residue was purified by Prep-HPLC (Condition 4, Gradient 4) to afford 2,7-dimethyl-5-[2-(piperidin-4-yl)-1,7-naphthyridin-6-yl]indazol-6-ol (1.4 mg, 6%) as a solid. LCMS (ES, m/z): 374 [M+H]⁺. ¹H NMR (400 MHz, DMSO-d₆) δ 13.30 (s, 1H), 9.41 (d, J=1.0 Hz, 1H), 8.75 (d, J=1.0 Hz, 2H), 8.50-8.44 (m, 2H), 8.37 (d, J=2.7 Hz, 2H), 7.81 (d, J=8.7 Hz, 1H), 4.15 (s, 3H), 3.41 (s, 1H), 3.26 (s, 2H), 3.12 (d, J=12.2 Hz, 2H), 2.39 (s, 3H), 2.18 (d, J=13.6 Hz, 2H), 2.06 (q, J=11.1 Hz, 2H).

### Example 107: Synthesis of Compound 283

### Synthesis of Intermediate C22

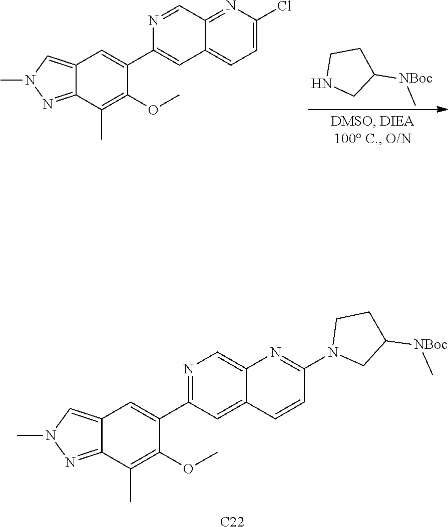

To a stirred mixture of 2-chloro-6-(6-methoxy-2,7-dimethylindazol-5-yl)-1,7-naphthyridine (250 mg, 0.73 mmol, 1 equiv) and tert-butyl N-methyl-N-(pyrrolidin-3-yl)carbamate (221.68 mg, 1.10 mmol, 1.5 equiv) in DMSO (3 mL) was added DIEA (286.11 mg, 2.21 mmol, 3 equiv) in portions at room temperature under nitrogen atmosphere. The resulting mixture was stirred overnight at 100° C. The reaction mixture was quenched with water at room temperature and extracted with ethyl acetate (2×5 mL). The organic layers were combined, washed with brine (2×10 mL), dried over anhydrous Na₂SO₄, and filtered. After filtration, the filtrate was concentrated under reduced pressure to afford tert-butyl N-{1-[6-(6-methoxy-2,7-dimethylindazol-5-yl)-1,7-naphthyridin-2-yl]pyrrolidin-3-yl}-N-methylcarbamate (30 mg, 8%) as a solid.

### Synthesis of Compound 283

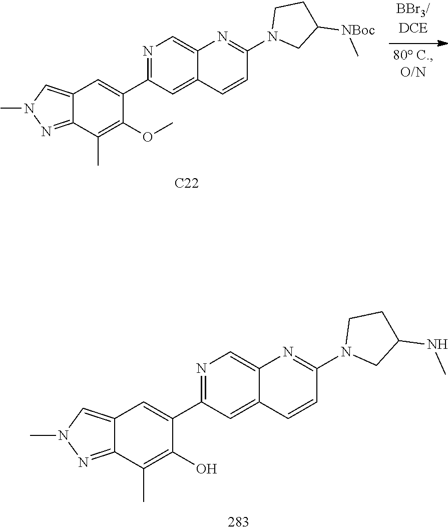

To a stirred solution of tert-butyl N-{1-[6-(6-methoxy-2,7-dimethylindazol-5-yl)-1,7-naphthyridin-2-yl]pyrrolidin-3-yl}-N-methylcarbamate (50 mg, 0.099 mmol, 1 equiv) in DCE (1 mL) was added BBr₃ (1 mL) dropwise at room temperature under nitrogen atmosphere. The resulting mixture was stirred overnight at 80° C., then quenched with methanol at 0° C., and the resulting mixture was filtered. After filtration, the filtrate was concentrated under reduced pressure to give a residue. The residue was purified by Prep-HPLC (Condition 4, Gradient 4) to afford 2,7-dimethyl-5-{2-[3-(methylamino)pyrrolidin-1-yl]-1,7-naphthyridin-6-yl}indazol-6-ol (1 mg, 3%) as a solid. LCMS (ES, m/z): 389 [M+H]⁺. ¹H NMR (400 MHz, DMSO-d₆) δ 13.78 (s, 1H), 8.90 (s, 1H), 8.48 (s, 1H), 8.29 (s, 1H), 8.22 (s, 1H), 8.12 (d, J=9.2 Hz, 1H), 7.23 (d, J=9.2 Hz, 1H), 4.12 (s, 3H), 3.76-3.68 (m, 3H), 3.45 (s, 1H), 3.22 (s, 1H), 2.34 (d, J=12.2 Hz, 3H), 2.30 (s, 3H), 2.12 (dq, J=13.1, 7.2 Hz, 1H), 1.99 (s, 2H).

### Example 108: Synthesis of Compound 284, 321, and 322

### Synthesis of Intermediate C23

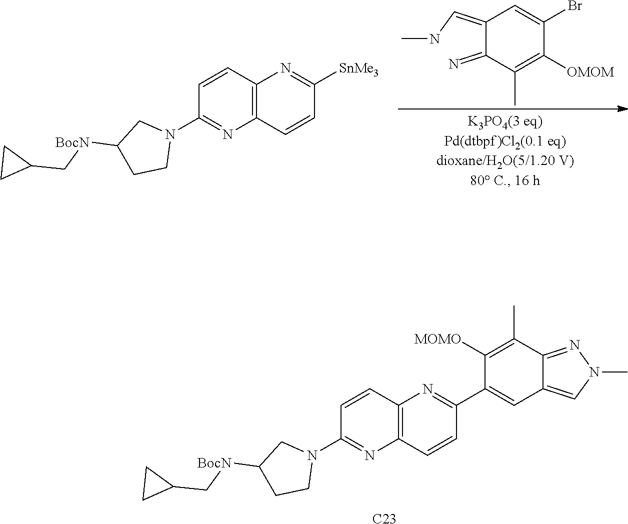

A mixture of tert-butyl N-(cyclopropylmethyl)-N-{1-[6-(trimethylstannyl)-1,5-naphthyridin-2-yl]pyrrolidin-3-yl}carbamate (600 mg, 1.107 mmol, 1 equiv), 5-bromo-6-(methoxymethoxy)-2,7-dimethylindazole (316 mg, 1.107 mmol, 1 equiv), Pd(DtBPF)Cl₂ (72 mg, 0.111 mmol, 0.1 equiv), and K₃PO₄ (705 mg, 3.321 mmol, 3 equiv) in dioxane (10 mL) and water (2 mL) was stirred for 16 hr at 80° C. under nitrogen atmosphere. The reaction mixture was cooled to room temperature, then poured into water (100 mL), and extracted with ethyl acetate (2×100 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-(cyclopropylmethyl)-N-(1-{6-[6-(methoxymethoxy)-2,7-dimethylindazol-5-yl]-1,5-naphthyridin-2-yl}pyrrolidin-3-yl)carbamate (190 mg, 30%) as a solid. LCMS (ES, m/z):573 [M+H]⁺.

### Synthesis of Compound 284

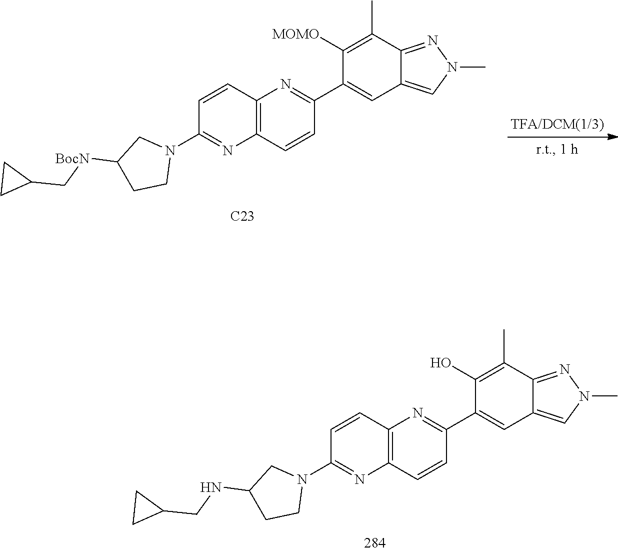

A solution of tert-butyl N-(cyclopropylmethyl)-N-(1-{6-[6-(methoxymethoxy)-2,7-dimethylindazol-5-yl]-1,5-naphthyridin-2-yl}pyrrolidin-3-yl)carbamate (190 mg, 0.332 mmol, 1 equiv) in TFA (1 mL) and DCM (3 mL) was stirred for 1 h at room temperature. The resulting mixture was concentrated under reduced pressure to give a residue. The residue was purified by Chiral-Prep-HPLC with the following conditions: Column, YMC-Actus Triart C18, 30×150 mm, Sum; mobile phase, water (10 mmol/L NH₄HCO₃) and ACN (30% ACN up to 70% in 8 min); Detector, UV 220 nm) to afford 5-(6-{3-[(cyclopropylmethyl)amino]pyrrolidin-1-yl}-1,5-naphthyridin-2-yl)-2,7-dimethylindazol-6-ol (9.8 mg, 7%) as a solid. LCMS (ES, m/z):429 [M+H]⁺. ¹H NMR (400 MHz, DMSO-d₆) δ 14.32 (s, 1H), 8.40 (t, J=4.7 Hz, 2H), 8.33 (s, 1H), 8.14 (d, J=9.2 Hz, 1H), 8.06 (d, J=9.1 Hz, 1H), 7.17 (d, J=9.3 Hz, 1H), 4.13 (s, 3H), 3.77 (dd, J=10.7, 6.1 Hz, 1H), 3.69-3.60 (m, 1H), 3.62-3.53 (m, 2H), 3.44-3.32 (m, 1H), 2.67-2.52 (m, 2H), 2.38 (s, 3H), 2.33-2.32 (m, 1H), 2.00-1.93 (s, 1H), 0.91 (dd, J=12.8, 6.4 Hz, 1H), 0.44 (dt, J=8.4, 2.9 Hz, 2H), 0.17 (d, J=4.8 Hz, 2H).

### Synthesis of Compounds 321 and 322

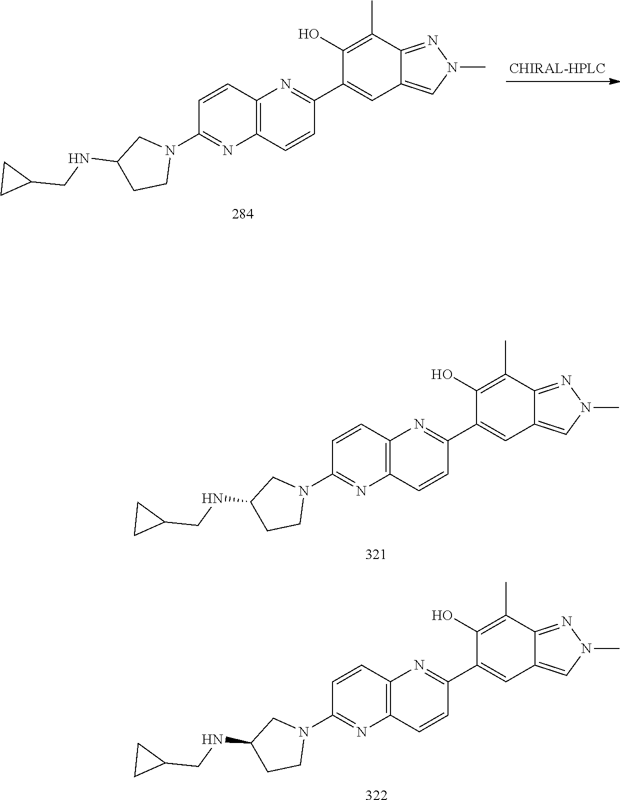 [SMILES: Cc1c(O)c(-c2ccc3nc(N4CCC(NCC5CC5)C4)ccc3n2)cc2cn(C)nc12 ; "284" ; Cc1c(O)c(-c2ccc3nc(N4CC[C@H](NCC5CC5)C4)ccc3n2)cc2cn(C)nc12 ; "321" ; Cc1c(O)c(-c2ccc3nc(N4CC[C@@H](NCC5CC5)C4)ccc3n2)cc2cn(C)nc12 ; "322"]

5-(6-{3-[(cyclopropylmethyl)amino]pyrrolidin-1-yl}-1,5-naphthyridin-2-yl)-2,7-dimethylindazol-6-ol was purified by chiral reverse flash chromatography (Condition 1, Gradient 1) to afford (22.3 mg) and (23.7 mg) as solids. Compound 321: LCMS (ES, m/z):429 [M+H]⁺. 1H NMR (400 MHz, DMSO-d₆) δ 14.33 (s, 1H), 8.43-8.36 (m, 2H), 8.33 (s, 1H), 8.13 (d, J=9.3 Hz, 1H), 8.06 (d, J=9.1 Hz, 1H), 7.16 (d, J=9.3 Hz, 1H), 4.13 (s, 3H), 3.75 (dd, J=10.8, 5.9 Hz, 1H), 3.68-3.60 (m, 1H), 3.57 (d, J=7.6 Hz, 2H), 3.44-3.32 (m, 1H), 2.49-2.46 (m, 2H), 2.38 (s, 3H), 2.14 (dd, J=12.6, 6.3 Hz, 1H), 1.92-1.88 (m, 1H), 0.88 (dd, J=12.8, 6.4 Hz, 1H), 0.46-0.37 (m, 2H), 0.18-0.10 (m, 2H). Compound 322: LCMS (ES, m/z):429 [M+H]⁺. ¹H NMR (400 MHz, DMSO-d₆) δ 14.33 (s, 1H), 8.40 (t, J=4.7 Hz, 2H), 8.33 (s, 1H), 8.13 (d, J=9.2 Hz, 1H), 8.06 (d, J=9.2 Hz, 1H), 7.16 (d, J=9.3 Hz, 1H), 4.13 (s, 3H), 3.78-3.70 (m, 1H), 3.68-3.60 (m, 1H), 3.58 (d, J=7.6 Hz, 2H), 3.47 (s, 1H), 2.49-2.47 (m, 2H), 2.38 (s, 3H), 2.14 (dd, J=12.6, 6.5 Hz, 1H), 1.88 (s, 1H), 0.89 (dd, J=12.8, 6.4 Hz, 1H), 0.42 (dt, J=8.8, 2.8 Hz, 2H), 0.18-0.10 (m, 2H).

### Example 109: Synthesis of Compound 302

### Synthesis of Intermediate C24

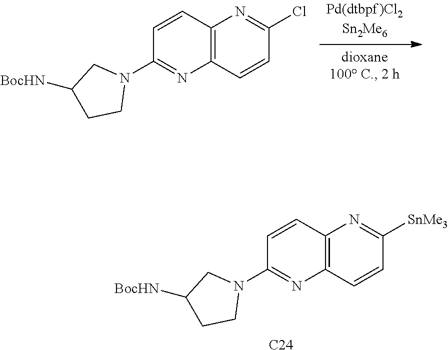

To a stirred mixture of tert-butyl N-[1-(6-chloro-1,5-naphthyridin-2-yl)pyrrolidin-3-yl]carbamate (200 mg, 0.573 mmol, 1 equiv) and Pd(dtbpf)Cl₂ (37.37 mg, 0.057 mmol, 0.1 equiv) in dioxane (4 mL) was added Sn₂Me₆ (375.69 mg, 1.146 mmol, 2 equiv) dropwise at room temperature under nitrogen atmosphere. The reaction mixture was stirred at 100° C. for 2 hr, then quenched with potassium fluoride solution (10 mL) at room temperature. The resulting mixture was filtered, and the aqueous layer was extracted with ethyl acetate (2×10 mL). The organic layers were combined, dried over Na₂SO₄, filtered, and concentrated under reduced pressure to afford tert-butyl N-{1-[6-(trimethylstannyl)-1,5-naphthyridin-2-yl]pyrrolidin-3-yl}carbamate (300 mg crude) as an oil.

### Synthesis of Intermediate C25

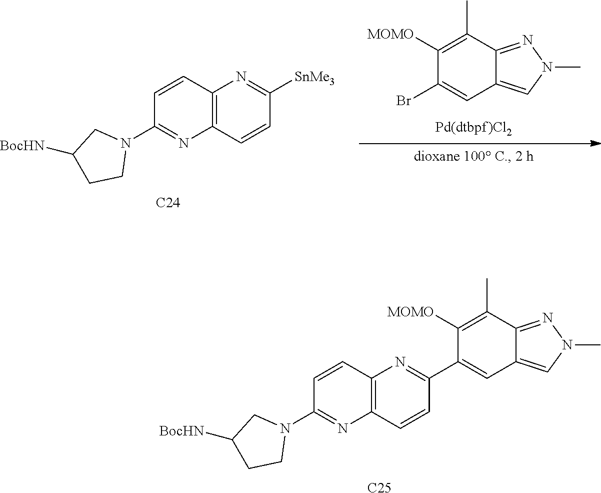

To a stirred mixture of tert-butyl N-{1-[6-(trimethylstannyl)-1,5-naphthyridin-2-yl]pyrrolidin-3-yl}carbamate (300 mg, 0.629 mmol, 1 equiv) and Pd(dtbpf)Cl₂ (40.97 mg, 0.063 mmol, 0.1 equiv) in dioxane (6 mL) was added 5-bromo-6-(methoxymethoxy)-2,7-dimethylindazole (215.11 mg, 0.755 mmol, 1.2 equiv) in portions at room temperature under nitrogen atmosphere. The mixture was stirred at 100° C. for 2 hr. The resulting mixture was concentrated under reduced pressure to give a residue. The residue was purified by silica gel column chromatography, eluted with CH₂Cl₂/MeOH (10:1) to afford tert-butyl N-(1-{6-[6-(methoxymethoxy)-2,7-dimethylindazol-5-yl]-1,5-naphthyridin-2-yl}pyrrolidin-3-yl)carbamate (150 mg, 46%) as a solid.

### Synthesis of Intermediate C26

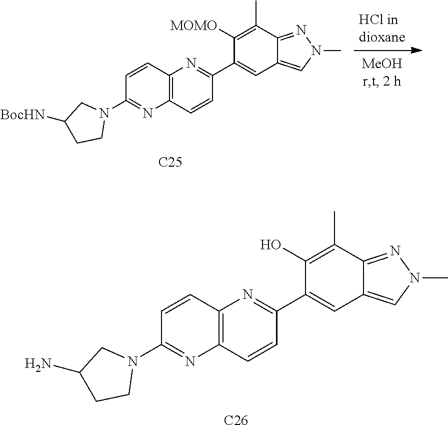

To a stirred solution of tert-butyl N-(1-{6-[6-(methoxymethoxy)-2,7-dimethylindazol-5-yl]-1,5-naphthyridin-2-yl}pyrrolidin-3-yl)carbamate (150 mg, 0.289 mmol, 1 equiv) in methanol (1.5 mL) was added HCl (gas) in 1,4-dioxane (1.5 mL) dropwise at room temperature under nitrogen atmosphere. The mixture was stirred at room temperature for 2 hr. The resulting mixture was concentrated under reduced pressure to afford 5-[6-(3-aminopyrrolidin-1-yl)-1,5-naphthyridin-2-yl]-2,7-dimethylindazol-6-ol (115 mg) as a solid.

### Synthesis of Compound 302

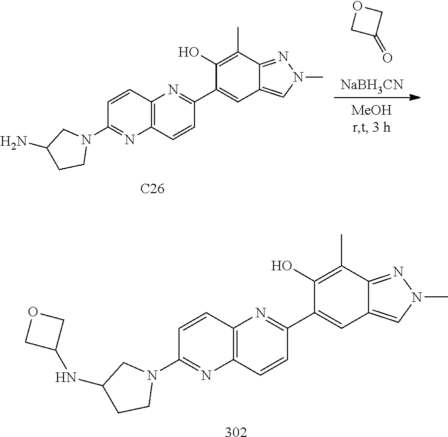

A solution of 5-[6-(3-aminopyrrolidin-1-yl)-1,5-naphthyridin-2-yl]-2,7-dimethylindazol-6-ol (90 mg, 0.240 mmol, 1 equiv) in methanol (1.8 mL) was treated with 3-oxetanone (25.98 mg, 0.360 mmol, 1.5 equiv) for 30 min at room temperature under nitrogen atmosphere. To the reaction mixture was added NaBH₃CN (45.31 mg, 0.720 mmol, 3 equiv) in portions at room temperature. The resulting mixture was stirred at room temperature for 3 hr, then concentrated under reduced pressure to give a residue. The residue was purified by Prep-HPLC (Condition 1, Gradient 15) to afford 2,7-dimethyl-5-{6-[3-(oxetan-3-ylamino)pyrrolidin-1-yl]-1,5-naphthyridin-2-yl}Indazol-6-ol (25.9 mg, 25%) as a solid. LCMS (ES, m/z): 431 [M+H]⁺. ¹H NMR (400 MHz, DMSO-d₆) δ 14.33 (s, 1H), 8.43-8.36 (m, 2H), 8.33 (s, 1H), 8.12 (d, J=9.2 Hz, 1H), 8.06 (d, J=9.1 Hz, 1H), 7.14 (d, J=9.3 Hz, 1H), 4.67 (td, J=6.5, 2.8 Hz, 2H), 4.36 (dt, J=8.7, 6.2 Hz, 2H), 4.13 (s, 3H), 4.00 (d, J=7.2 Hz, 1H), 3.69 (dd, J=10.9, 5.9 Hz, 2H), 3.55 (s, 1H), 3.35 (s, 1H), 2.80 (s, 1H), 2.38 (s, 3H), 2.13-2.01 (m, 1H), 1.79 (dd, J=12.6, 6.5 Hz, 1H).

### Example 110: Synthesis of Compounds 315-317

### Synthesis of Intermediate C27

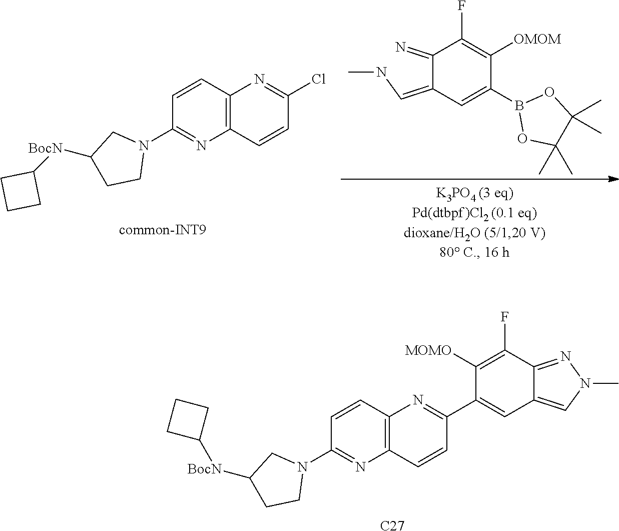

A mixture of tert-butyl N-[1-(6-chloro-1,5-naphthyridin-2-yl)pyrrolidin-3-yl]-N-cyclobutylcarbamate (400 mg, 0.993 mmol, 1 equiv), 7-fluoro-6-(methoxymethoxy)-2-methyl-5-(4,4,5,5-tetramethyl-1,3,2-dioxaborolan-2-yl)indazole (834 mg, 2.482 mmol, 2.5 equiv), K₃PO₄ (632 mg, 2.979 mmol, 3 equiv), and Pd(DtBPF)Cl₂ (65 mg, 0.099 mmol, 0.1 equiv) in 1,4-dioxane (6.7 mL) and water (1.3 mL) was stirred for 16 hr at 80° C. under nitrogen atmosphere. The reaction mixture was cooled to room temperature, then extracted with ethyl acetate (1×40 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:1), followed by Prep-HPLC (Condition 2, Gradient 10) to afford tert-butyl N-cyclobutyl-N-(1-{6-[7-fluoro-6-(methoxymethoxy)-2-methylindazol-5-yl]-1,5-naphthyridin-2-yl}pyrrolidin-3-yl)carbamate (158 mg, 28%) as a solid. LCMS (ES, m/z): 577 [M+H]⁺.

### Synthesis of Compound 315

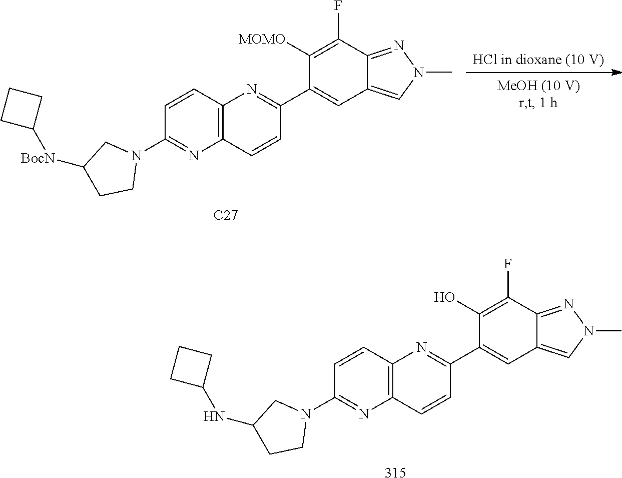

A mixture of tert-butyl N-cyclobutyl-N-(1-{6-[7-fluoro-6-(methoxymethoxy)-2-methylindazol-5-yl]-1,5-naphthyridin-2-yl}pyrrolidin-3-yl)carbamate (28 mg, 0.049 mmol, 1 equiv) in methanol (1 mL) and HCl (gas) in 1,4-dioxane (1 mL) was stirred for 1 h at room temperature. The resulting mixture was concentrated under reduced pressure to give a residue. The residue was purified by Prep-HPLC (Condition 3, Gradient 21) to afford 5-{6-[3-(cyclobutylamino)pyrrolidin-1-yl]-1,5-naphthyridin-2-yl}-7-fluoro-2-methylindazol-6-ol (5.6 mg, 26%) as a solid. LCMS (ES, m/z): 433 [M+H]⁺. ¹H NMR (400 MHz, DMSO-d₆) δ 14.44 (s, 1H), 8.48 (d, J=2.7 Hz, 1H), 8.45-8.38 (m, 2H), 8.14 (d, J=22.9, 9.2 Hz, 1H), 8.08 (d, J=22.9, 9.2 Hz, 1H), 7.16 (d, J=9.3 Hz, 1H), 4.16 (s, 3H), 3.69 (s, 2H), 3.56 (s, 1H), 3.40 (s, 1H), 3.26 (s, 2H), 2.14 (s, 3H), 1.83 (s, 1H), 1.71 (s, 2H), 1.59 (d, J=18.6, 9.3, 8.3 Hz, 2H).

### Synthesis of Intermediates C28 and C29

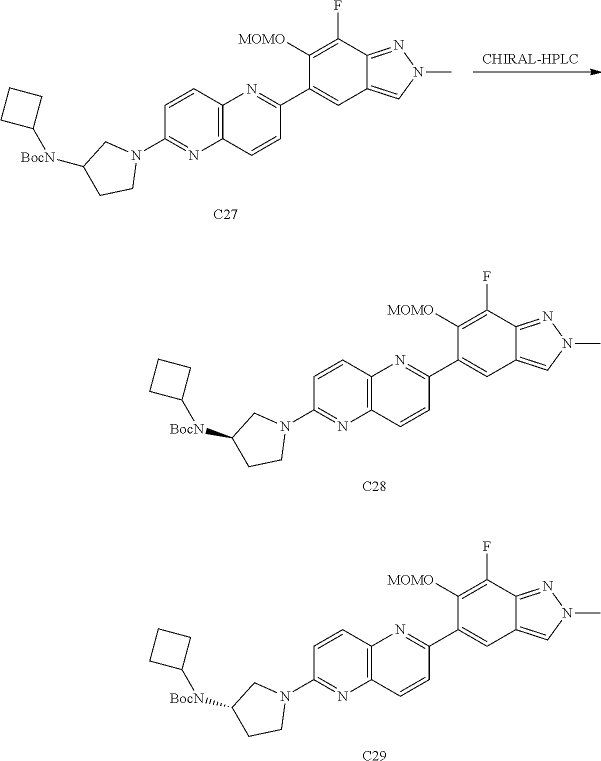

5-{6-[3-(cyclobutylamino)pyrrolidin-1-yl]-1,5-naphthyridin-2-yl}-7-fluoro-2-methylindazol-6-ol was purified by Prep-HPLC (Column: CHIRAL ART Cellulose-SB, 2×25 cm, 5 m; Mobile Phase A: MtBE(0.1% DEA)-HPLC, Mobile Phase B: MeOH-HPLC; Flow rate: 20 mL/min; Gradient: 30% B to 30% B in 6.5 min; Wave Length: 220/254 nm; RT1(min): 5.1; RT2(min): 5.9; Sample Solvent: MeOH: DCM=8: 1; Injection Volume: 0.3 mL; Number Of Runs: 32) to afford (First peak) (59 mg) and (Second peak)(63 mg) as solids. LCMS: (ES, m/z): 577 [M+H]⁺.

### Synthesis of Compound 316

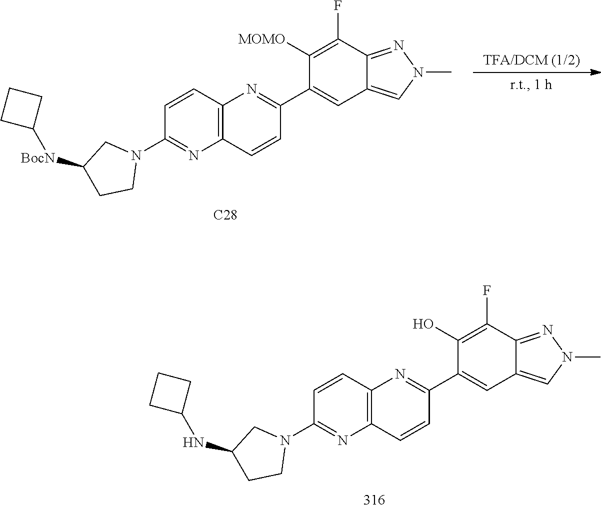

A mixture of tert-butyl N-cyclobutyl-N-[(3R)-1-{6-[7-fluoro-6-(methoxymethoxy)-2-methylindazol-5-yl]-1,5-naphthyridin-2-yl}pyrrolidin-3-yl]carbamate (59 mg, 0.102 mmol, 1 equiv) in DCM (2 mL) and TFA (1 mL) was stirred for 1 hr at room temperature. The resulting mixture was concentrated under reduced pressure to give a residue. The residue was purified by Prep-HPLC (Condition 3, Gradient 20) to afford Compound 316 (26 mg, 58%) as a solid. LCMS (ES, m/z): 433 [M+H]⁺. ¹H NMR (400 MHz, DMSO-d₆) δ 14.44 (s, 1H), 8.48 (d, J=2.7 Hz, 1H), 8.44-8.38 (m, 2H), 8.14 (d, J=9.4 Hz, 1H), 8.08 (d, J=9.1 Hz, 1H), 7.16 (d, J=9.3 Hz, 1H), 4.16 (s, 3H), 3.69 (s, 2H), 3.56 (s, 1H), 3.38 (m, 1H), 3.27 (m, 1H), 2.12 (ddd, J=18.2, 9.3, 4.9 Hz, 4H), 1.83 (s, 1H), 1.70 (dd, J=11.4, 8.2 Hz, 2H), 1.64-1.50 (m, 2H).

### Synthesis of Compound 317

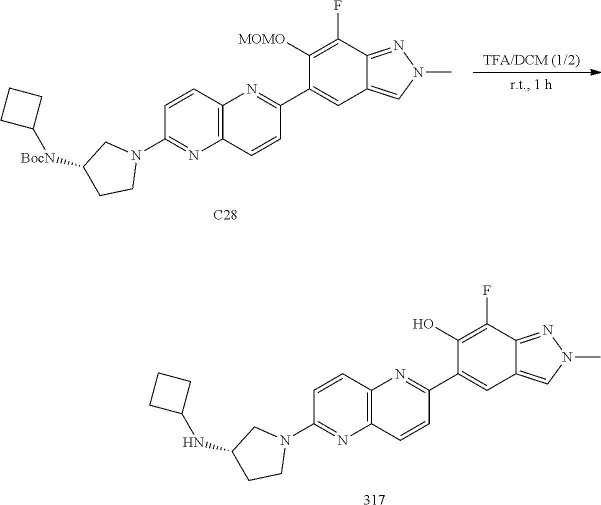

A mixture of tert-butyl N-cyclobutyl-N-[(3S)-1-{6-[7-fluoro-6-(methoxymethoxy)-2-methylindazol-5-yl]-1,5-naphthyridin-2-yl}pyrrolidin-3-yl]carbamate (63 mg, 0.109 mmol, 1 equiv) in DCM (2 mL) and TFA (1 mL) was stirred for 1 h at room temperature. The resulting mixture was concentrated under reduced pressure to give a residue. The residue was purified by Prep-HPLC (Condition 3, Gradient 20) to afford Compound 317 (26 mg, 55%) as a solid. LCMS (ES, m/z): 433 [M+H]⁺. ¹H NMR (400 MHz, DMSO-d₆) δ 14.44 (s, 1H), 8.48 (d, J=2.6 Hz, 1H), 8.44-8.38 (m, 2H), 8.11 (d, J=22.0, 9.3 Hz, H), 8.08 (d, J=9.1 Hz, 1H), 7.16 (d, J=9.3 Hz, 1H), 4.16 (s, 3H), 3.69 (s, 2H), 3.56 (s, 1H), 3.38 (m, 1H), 3.27 (m, 1H), 2.12 (ddt, J=18.1, 11.9, 5.1 Hz, 4H), 1.82 (s, 1H), 1.69 (m, 2H), 1.58 (m, 2H).

### Example 111: Synthesis of Compounds 294, 303, and 304

### Synthesis of Intermediate C29

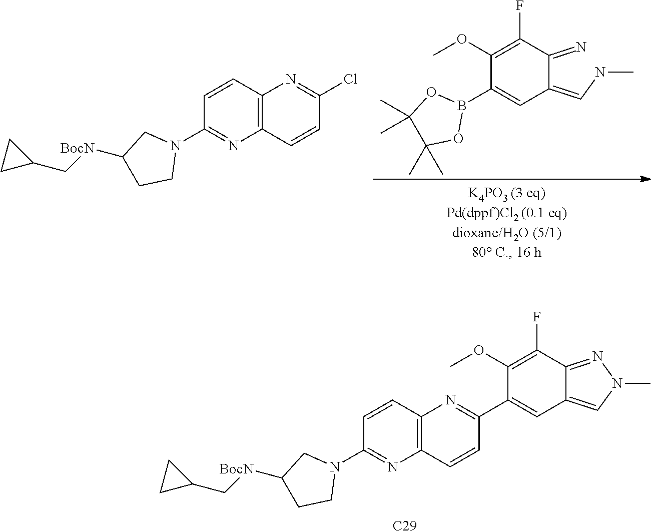

A mixture of tert-butyl N-[1-(6-chloro-1,5-naphthyridin-2-yl)pyrrolidin-3-yl]-N-(cyclopropylmethyl)carbamate (500 mg, 1.241 mmol, 1 equiv), 7-fluoro-6-methoxy-2-methyl-5-(4,4,5,5-tetramethyl-1,3,2-dioxaborolan-2-yl)indazole (380 mg, 1.241 mmol, 1 equiv), Pd(DtBPF)Cl₂ (81 mg, 0.124 mmol, 0.1 equiv), and K₃PO₄ (790 mg, 3.723 mmol, 3 equiv) in dioxane (10 mL) and water (2 mL) was stirred for 16 h at 80° C. under nitrogen atmosphere. The reaction mixture cooled to room temperature, poured into water (20 mL), and extracted with ethyl acetate (3×20 mL). The organic layers were combined, dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-(cyclopropylmethyl)-N-{1-[6-(7-fluoro-6-methoxy-2-methylindazol-5-yl)-1,5-naphthyridin-2-yl]pyrrolidin-3-yl}carbamate (300 mg, 44%) as a solid. LCMS (ES, m/z):547 [M+H]⁺.

### Synthesis of Compound 294

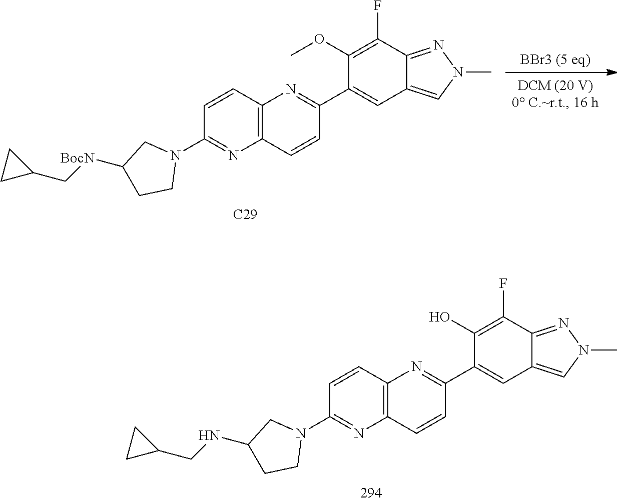 [SMILES: COc1c(-c2ccc3nc(N4CCC(N(CC5CC5)C(=O)OC(C)(C)C)C4)ccc3n2)cc2cn(C)nc2c1F ; "C29" ; Cn1cc2cc(-c3ccc4nc(N5CCC(NCC6CC6)C5)ccc4n3)c(O)c(F)c2n1 ; "294"]

To a stirred solution of tert-butyl N-(cyclopropylmethyl)-N-{1-[6-(7-fluoro-6-methoxy-2-methylindazol-5-yl)-1,5-naphthyridin-2-yl]pyrrolidin-3-yl}carbamate (300 mg, 0.549 mmol, 1 equiv) in DCM (6 mL) was added BBr₃ (687 mg, 2.745 mmol, 5 equiv) dropwise at 0° C. The resulting mixture was stirred for 16 h at room temperature. The reaction mixture was quenched with methanol (30 mL) at 0° C., then concentrated under reduced pressure and basified to pH 8 with saturated NaHCO₃ (aq.). The resulting mixture was extracted with CH₂Cl₂ (3×50 mL). The organic layers were combined, dried over anhydrous Na₂SO₄, and filtered. After filtration, the filtrate was concentrated under reduced pressure to give a residue. The residue was purified by Prep-HPLC (Condition 3, Gradient 22) to afford 5-(6-{3-[(cyclopropylmethyl)amino]pyrrolidin-1-yl}-1,5-naphthyridin-2-yl)-7-fluoro-2-methylindazol-6-ol (75 mg) as a solid. LCMS (ES, m/z):433 [M+H]⁺. ¹H NMR (400 MHz, DMSO-d₆) δ 14.44 (s, 1H), 8.48 (d, J=2.7 Hz, 1H), 8.44-8.37 (m, 2H), 8.14 (dd, J=9.3, 0.8 Hz, 1H), 8.11-8.05 (m, 1H), 7.17 (d, J=9.3 Hz, 1H), 4.16 (s, 3H), 3.82-3.68 (m, 3H), 3.57 (d, J=8.8 Hz, 1H), 3.45 (t, J=5.4 Hz, 1H), 2.46 (d, J=6.6 Hz, 2H), 2.13 (dq, J=12.9, 6.6 Hz, 1H), 1.88 (dd, J=12.3, 6.2 Hz, 2H), 0.96-0.82 (m, 1H), 0.46-0.35 (m, 2H), 0.17-0.05 (m, 2H).

### Synthesis of Compounds 303 and 304

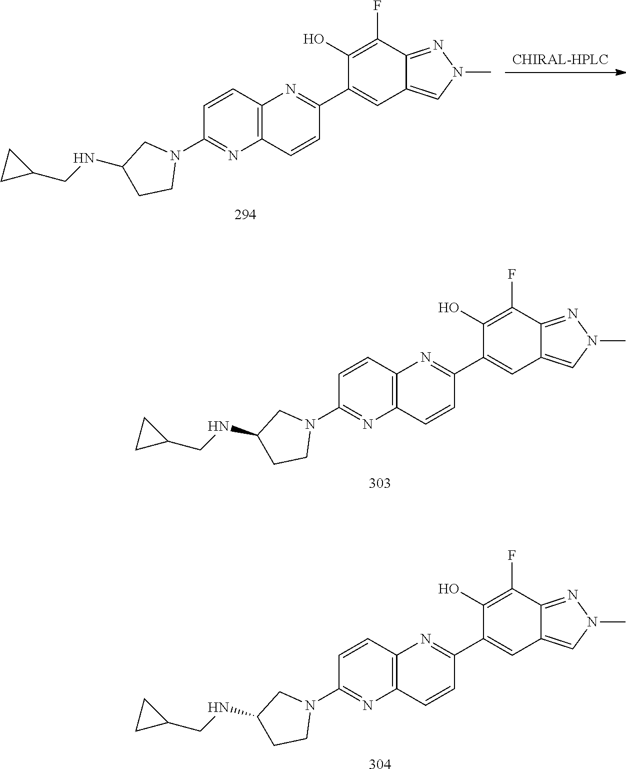 [SMILES: Cn1cc2cc(-c3ccc4nc(N5CCC(NCC6CC6)C5)ccc4n3)c(O)c(F)c2n1 ; "294" ; Cn1cc2cc(-c3ccc4nc(N5CC[C@@H](NCC6CC6)C5)ccc4n3)c(O)c(F)c2n1 ; "303" ; Cn1cc2cc(-c3ccc4nc(N5CC[C@H](NCC6CC6)C5)ccc4n3)c(O)c(F)c2n1 ; "304"]

5-(6-{3-[(cyclopropylmethyl)amino]pyrrolidin-1-yl}-1,5-naphthyridin-2-yl)-7-fluoro-2-methylindazol-6-ol was purified by chiral prep-HPLC (CHIRAL ART Cellulose-SB, 2×25 cm, 5 um; Mobile Phase A: MtBE (0.1% DEA)-HPLC, Mobile Phase B: MeOH-HPLC; Flow rate: 20 mL/min; Gradient: 28% B to 28% B in 7 min; Wave Length: UV 220/254 nm; RT1(min): 6.1; RT2(min): 6.7; Sample Solvent: MeOH: DCM=1: 2; Injection Volume: 0.24 mL; Number Of Runs: 28) to afford the compounds as solids. Compound 303: LCMS (ES, m/z):433 [M+H]⁺. 1H NMR (400 MHz, DMSO-d₆) δ 14.44 (s, 1H), 8.48 (d, J=2.7 Hz, 1H), 8.44-8.37 (m, 2H), 8.11 (dd, J=21.8, 9.2 Hz, 2H), 7.17 (d, J=9.3 Hz, 1H), 4.16 (s, 3H), 3.74 (dd, J=10.8, 5.8 Hz, 1H), 3.68 (s, 1H), 3.58 (s, 1H), 3.48-3.41 (m, 2H), 2.46 (d, J=6.7 Hz, 2H), 2.13 (dq, J=12.9, 6.7 Hz, 1H), 1.97 (s, 1H), 1.91-1.83 (m, 1H), 0.96-0.82 (m, 1H), 0.46-0.35 (m, 2H), 0.17-0.09 (m, 2H). Compound 304: LCMS (ES, m/z):433 [M+H]⁺. ¹H NMR (400 MHz, DMSO-d₆) δ 14.44 (s, 1H), 8.48 (d, J=2.7 Hz, 1H), 8.45-8.38 (m, 2H), 8.15 (d, J=9.3 Hz, 1H), 8.09 (d, J=9.1 Hz, 1H), 7.18 (d, J=9.3 Hz, 1H), 4.16 (s, 3H), 3.74 (d, J=7.5 Hz, 1H), 3.69 (s, 1H), 3.49 (s, 1H), 3.58 (s, 1H), 3.48-3.41 (m, 2H), 2.46 (d, J=6.7 Hz, 2H), 2.15 (dd, J=12.4, 6.4 Hz, 1H), 1.90 (s, 1H), 0.89 (s, 1H), 0.42 (dt, J=8.4, 2.8 Hz, 2H), 0.19-0.11 (m, 2H).

### Example 112: Synthesis of Compounds 285, 286, and 388

### Synthesis of Compound 388

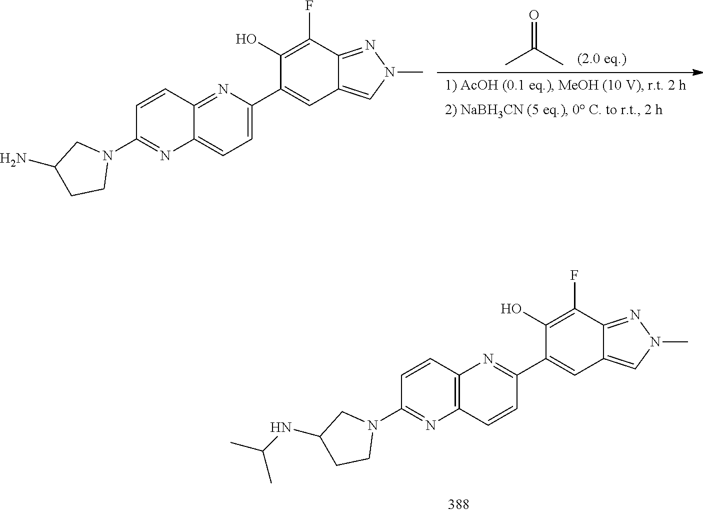

A mixture of 5-[6-(3-aminopyrrolidin-1-yl)-1,5-naphthyridin-2-yl]-7-fluoro-2-methylindazol-6-ol (280 mg, 0.74 mmol, 1.00 equiv), acetone (85.95 mg, 1.48 mmol, 2.00 equiv), AcOH (4.44 mg, 0.07 mmol, 0.10 equiv), and methanol (20 mL) was stirred for 2 h at room temperature under nitrogen atmosphere. To the reaction mixture was added NaBH₃CN (232.50 mg, 3.70 mmol, 5.00 equiv) dropwise at 0° C. The resulting mixture was stirred for 2 hr at room temperature, then concentrated under reduced pressure to give a residue. The residue was purified by Prep-HPLC (Condition 3, Gradient 16) to afford 7-fluoro-5-{6-[3-(isopropylamino) pyrrolidin-1-yl]-1,5-naphthyridin-2-yl}-2-methylindazol-6-ol (65 mg, 21%) as a solid. LCMS (ES, m/z):421 [M+H]⁺.

### Synthesis of Compound 285

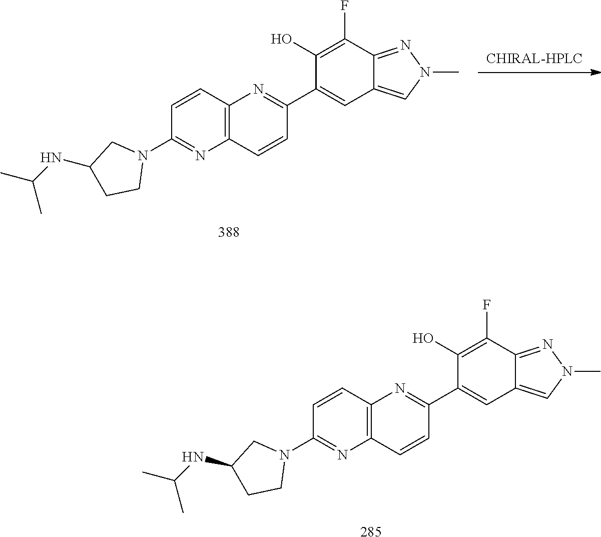

7-fluoro-5-{6-[3-(isopropylamino)pyrrolidin-1-yl]-1,5-naphthyridin-2-yl}-2-methylindazol-6-ol (65 mg, 0.155 mmol, 1 equiv) was purified by reverse flash chromatography (column, CHIRAL ART Cellulose-SB, 4.6*100 mm, 3 m; Mobile Phase A: MtBE(0.1% DEA): MeOH=70: 30; Flow rate: 1 mL/min) to afford 7-fluoro-5-{6-[(3R)-3-(isopropylamino) pyrrolidin-1-yl]-1,5-naphthyridin-2-yl}-2-methylindazol-6-ol (27 mg, 41%) as a solid. LCMS (ES, m/z):421 [M+H]⁺. 1H NMR (400 MHz, DMSO-d₆) δ 14.44 (s, 1H), 8.48 (d, J=2.7 Hz, 1H), 8.44-8.38 (m, 2H), 8.11 (dd, J=22.1, 9.2 Hz, 2H), 7.17 (d, J=9.3 Hz, 1H), 4.16 (s, 2H), 3.80 (s, 1H), 3.71 (s, 1H), 3.55 (d, J=9.9 Hz, 2H), 2.88 (s, 1H), 2.48 (s, 5H), 2.16 (s, 1H), 1.82 (s, 1H), 1.03 (dd, J=6.2, 4.5 Hz, 5H).

### Synthesis of Compound 286

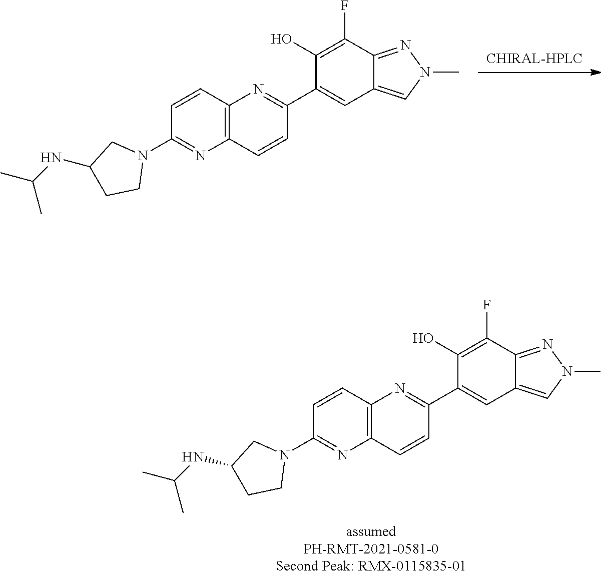

7-fluoro-5-{6-[3-(isopropylamino)pyrrolidin-1-yl]-1,5-naphthyridin-2-yl}-2-methylindazol-6-ol (65 mg, 0.155 mmol, 1 equiv) was purified by chiral reverse flash chromatography (column, CHIRAL ART Cellulose-SB, 4.6*100 mm, 3 m; Mobile Phase A: MtBE (0.1% DEA): MeOH=70: 30; Flow rate: 1 mL/min) to afford 7-fluoro-5-{6-[(3S)-3-(isopropylamino)pyrrolidin-1-yl]-1,5-naphthyridin-2-yl}-2-methylindazol-6-ol (27.1 mg, 41%) as a solid. LCMS (ES, m/z):421 [M+H]⁺. ¹H NMR (400 MHz, DMSO-d₆) δ 14.44 (s, 1H), 8.48 (d, J=2.7 Hz, 1H), 8.44-8.38 (m, 2H), 8.14 (d, J=9.2 Hz, 1H), 8.08 (d, J=9.1 Hz, 1H), 7.17 (d, J=9.3 Hz, 1H), 4.16 (s, 3H), 3.81 (s, 1H), 3.71 (s, 1H), 3.55 (dd, J=11.8, 5.3 Hz, 2H), 3.21 (s, 1H), 2.91 (s, 1H), 2.18 (q, J=6.8 Hz, 1H), 1.84 (s, 1H), 1.04 (dd, J=6.2, 4.4 Hz, 6H).

### Example 113: Synthesis of Compound 389, 451, and 452

### Synthesis of Intermediate C30

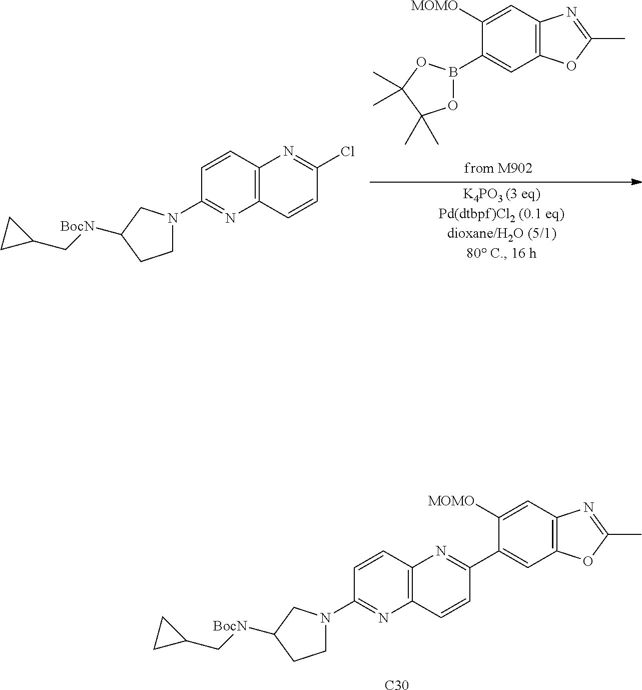

A mixture of tert-butyl N-[1-(6-chloro-1,5-naphthyridin-2-yl)pyrrolidin-3-yl]-N-(cyclopropylmethyl)carbamate (500 mg, 1.241 mmol, 1 equiv), 5-(methoxymethoxy)-2-methyl-6-(4,4,5,5-tetramethyl-1,3,2-dioxaborolan-2-yl)-1,3-benzoxazole (396 mg, 1.241 mmol, 1.00 equiv), Pd(DtBPF)Cl₂ (81 mg, 0.124 mmol, 0.1 equiv), and K₃PO₄ (790 mg, 3.723 mmol, 3 equiv) in dioxane (10 mL) and water (2 mL) was stirred for 16 hr at 80° C. under nitrogen atmosphere. The resulting mixture was cooled to room temperature, then poured into water (20 mL), extracted with ethyl acetate (3×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-(cyclopropylmethyl)-N-(1-{6-[5-(methoxymethoxy)-2-methyl-1,3-benzoxazol-6-yl]-1,5-naphthyridin-2-yl}pyrrolidin-3-yl)carbamate (350 mg, 50%) as a solid. LCMS (ES, m/z):560 [M+H]⁺.

### Synthesis of Compound 389

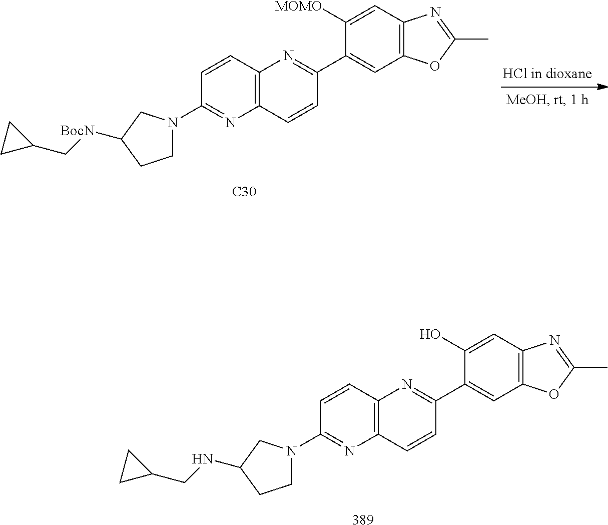

A mixture of tert-butyl N-(cyclopropylmethyl)-N-(1-{6-[5-(methoxymethoxy)-2-methyl-1,3-benzoxazol-6-yl]-1,5-naphthyridin-2-yl}pyrrolidin-3-yl)carbamate (350 mg, 0.625 mmol, 1 equiv) and HCl (gas) in 1,4-dioxane (1 mL) and methanol (2 mL) was stirred for 1 h at room temperature. The resulting mixture was concentrated under reduced pressure to give a residue. The residue was purified by Prep-HPLC (Condition 2, Gradient 12) to afford 6-(6-{3-[(cyclopropylmethyl)amino]pyrrolidin-1-yl}-1,5-naphthyridin-2-yl)-2-methyl-1,3-benzoxazol-5-ol (110 mg) as a solid. LCMS (ES, m/z):416 [M+H]⁺. ¹H NMR (400 MHz, DMSO-d₆) δ 14.53 (s, 1H), 8.45-8.39 (m, 2H), 8.10 (dd, J=15.2, 9.2 Hz, 2H), 7.16 (d, J=9.3 Hz, 1H), 7.12 (s, 1H), 3.74 (dd, J=10.8, 5.9 Hz, 1H), 3.68 (s, 1H), 3.58 (s, 1H), 3.48-3.41 (m, 1H), 2.61 (s, 3H), 2.46 (d, J=6.7 Hz, 2H), 2.13 (dq, J=12.8, 6.6 Hz, 1H), 1.88-1.85 (m, 2H), 0.96-0.82 (m, 1H), 0.45-0.37 (m, 2H), 0.17-0.05 (m, 2H).

### Synthesis of Compounds 451 and 452

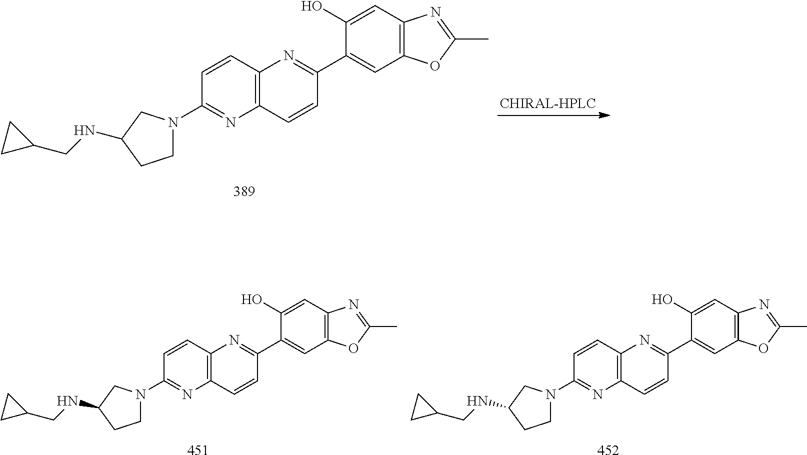 [SMILES: Cc1nc2cc(O)c(-c3ccc4nc(N5CCC(NCC6CC6)C5)ccc4n3)cc2o1 ; "389" ; Cc1nc2cc(O)c(-c3ccc4nc(N5CC[C@@H](NCC6CC6)C5)ccc4n3)cc2o1 ; Cc1nc2cc(O)c(-c3ccc4nc(N5CC[C@H](NCC6CC6)C5)ccc4n3)cc2o1 ; "451" ; "452"]

6-(6-{3-[(cyclopropylmethyl)amino]pyrrolidin-1-yl}-1,5-naphthyridin-2-yl)-2-methyl-1,3-benzoxazol-5-ol (90 mg, 0.217 mmol, 1 equiv) was purified by chiral prep-HPLC (Condition 4, Gradient 1) to afford Compound 451 (First peak) (17.7 mg) and Compound 452 (Second peak) (18.5 mg) as solids. Compound 451: LCMS (ES, m/z):416 [M+H]⁺. ¹H NMR (400 MHz, DMSO-d₆) δ 14.53 (s, 1H), 8.46-8.39 (m, 2H), 8.11 (dd, J=17.2, 9.2 Hz, 2H), 7.17 (d, J=9.3 Hz, 1H), 7.12 (s, 1H), 3.75 (dd, J=9.9, 5.4 Hz, 1H), 3.68 (s, 1H), 3.58 (d, J=8.0 Hz, 1H), 3.57-3.48 (m, 1H), 3.47-3.38 (m, 1H), 2.61 (s, 3H), 2.48 (s, 2H), 2.20-2.10 (m, 1H), 1.89-1.88 (m, 1H), 0.95-0.85 (m, 1H), 0.47-0.38 (m, 2H), 0.19-0.10 (m, 2H). Compound 452: LCMS (ES, m/z):416 [M+H]⁺. ¹H NMR (400 MHz, DMSO-d₆) δ 14.54 (s, 1H), 8.46-8.39 (m, 2H), 8.16-8.05 (m, 2H), 7.17 (d, J=9.3 Hz, 1H), 7.12 (s, 1H), 3.78-3.66 (m, 2H), 3.58 (d, J=8.0 Hz, 1H), 3.57-3.48 (m, 1H), 3.47-3.38 (m, 1H), 2.61 (s, 3H), 2.47 (d, J=6.4 Hz, 2H), 2.14 (dd, J=12.5, 6.3 Hz, 1H), 1.89-1.88 (m, 1H), 0.94-0.84 (m, 1H), 0.46-0.37 (m, 2H), 0.18-0.10 (m, 2H).

### Example 114: Synthesis of Compound 293, 307, and 308

### Synthesis of Intermediate C31

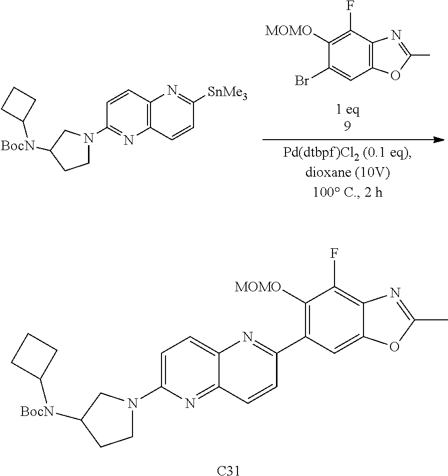

A mixture of 6-bromo-4-fluoro-5-(methoxymethoxy)-2-methyl-1,3-benzoxazole (250 mg, 0.862 mmol, 1 equiv), tert-butyl N-cyclobutyl-N-{1-[6-(trimethylstannyl)-1,5-naphthyridin-2-yl]pyrrolidin-3-yl}carbamate (687 mg, 1.293 mmol, 1.5 equiv), and Pd(DtBPF)Cl₂ (56 mg, 0.086 mmol, 0.1 equiv) in 1,4-dioxane (2.5 mL) was stirred for 2 hr at 100° C. under nitrogen atmosphere. The reaction mixture was cooled to room temperature, then poured into the water (10 mL), extracted with ethyl acetate (1×10 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-cyclobutyl-N-(1-{6-[4-fluoro-5-(methoxymethoxy)-2-methyl-1,3-benzoxazol-6-yl]-1,5-naphthyridin-2-yl}pyrrolidin-3-yl)carbamate (200 mg, 40%) as a solid. LCMS (ES, m/z): 578 [M+H]⁺.

### Synthesis of Compound 293

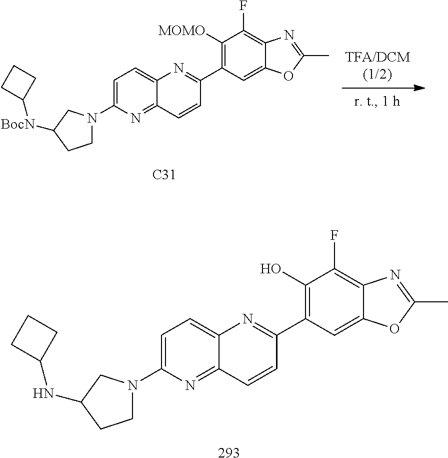

A mixture of tert-butyl N-cyclobutyl-N-(1-{6-[4-fluoro-5-(methoxymethoxy)-2-methyl-1,3-benzoxazol-6-yl]-1,5-naphthyridin-2-yl}pyrrolidin-3-yl)carbamate (200 mg, 0.346 mmol, 1 equiv) in DCM (2 mL) and CF₃COOH (1 mL) was stirred for 1 hr at room temperature. The resulting mixture was concentrated under reduced pressure to give a residue. The residue was purified by (Condition 3, Gradient 23) to afford 6-{6-[3-(cyclobutylamino)pyrrolidin-1-yl]-1,5-naphthyridin-2-yl}-4-fluoro-2-methyl-1,3-benzoxazol-5-ol (12 mg, 8%) as a solid. LCMS (ES, m/z): 434 [M+H]⁺. ¹H NMR (400 MHz, DMSO-d₆) δ 15.08 (s, 1H), 8.45 (d, J=9.3 Hz, 1H), 8.34 (d, J=1.4 Hz, 1H), 8.17 (d, J=9.3 Hz, 1H), 8.11 (d, J=9.2 Hz, 2H), 7.18 (d, J=9.3 Hz, 1H), 3.69 (s, 2H), 3.55 (s, 1H), 3.37 (s, 1H), 3.29 (s, 2H), 2.64 (s, 3H), 2.18-2.06 (m, 4H), 1.83 (s, 1H), 1.74-1.63 (m, 2H), 1.63-1.50 (m, 2H).

### Synthesis of Compounds 307 and 308

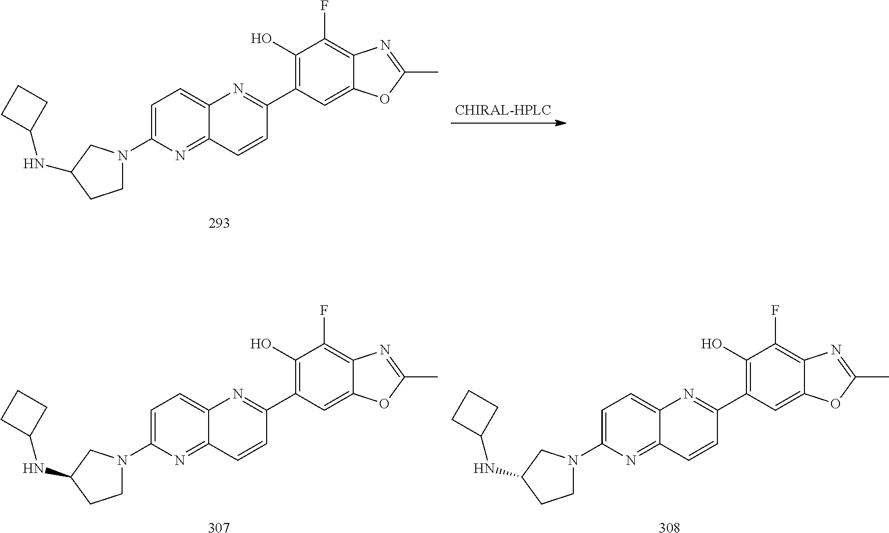

6-{6-[3-(cyclobutylamino)pyrrolidin-1-yl]-1,5-naphthyridin-2-yl}-4-fluoro-2-methyl-1,3-benzoxazol-5-ol was purified by chiral prep-HPLC (Column: CHIRAL ART Cellulose-SB, 2×25 cm, 5 m; Mobile Phase A: MtBE(0.1% DEA)-HPLC, Mobile Phase B: MeOH-HPLC; Flow rate: 20 mL/min; Gradient: 30% B to 30% B in 7.5 min) to afford Compound 307 (First peak) (32 mg, 41%) and Compound 308 (30.5 mg, 39%) as solids. Compound 307: LCMS (ES, m/z): 434 [M+H]⁺. ¹H NMR (400 MHz, DMSO-d₆) δ 15.08 (s, 1H), 8.45 (d, J=9.2 Hz, 1H), 8.34 (d, J=1.4 Hz, 1H), 8.14 (d, J=22.3, 9.3 Hz, 1H), 8.08 (d, J=22.3, 9.3 Hz, 1H), 7.18 (d, J=9.3 Hz, 1H), 3.70 (s, 2H), 3.56 (s, 1H), 3.36 (s, 1H), 3.25 (s, 2H), 2.64 (s, 3H), 2.18-2.05 (m, 4H), 1.83 (s, 1H), 1.70 (t, J=9.8 Hz, 2H), 1.66-1.50 (m, 2H). Compound 308: LCMS (ES, m/z): 434 [M+H]⁺. ¹H NMR (400 MHz, DMSO-d₆) δ 15.08 (s, 1H), 8.45 (d, J=9.2 Hz, 1H), 8.34 (d, J=1.4 Hz, 1H), 8.17 (d, J=9.3 Hz, 1H), 8.11 (d, J=9.1 Hz, 1H), 7.18 (d, J=9.3 Hz, 1H), 3.70 (s, 2H), 3.56 (s, 1H), 3.36 (s, 1H), 3.25 (s, 2H), 2.64 (s, 3H), 2.18-2.10 (m, 4H), 1.83 (s, 1H), 1.70 (s, 2H), 1.57 (dt, J=18.4, 8.8 Hz, 2H).

### Example 115: Synthesis of Compound 294, 323, and 324

### Synthesis of Intermediate C32

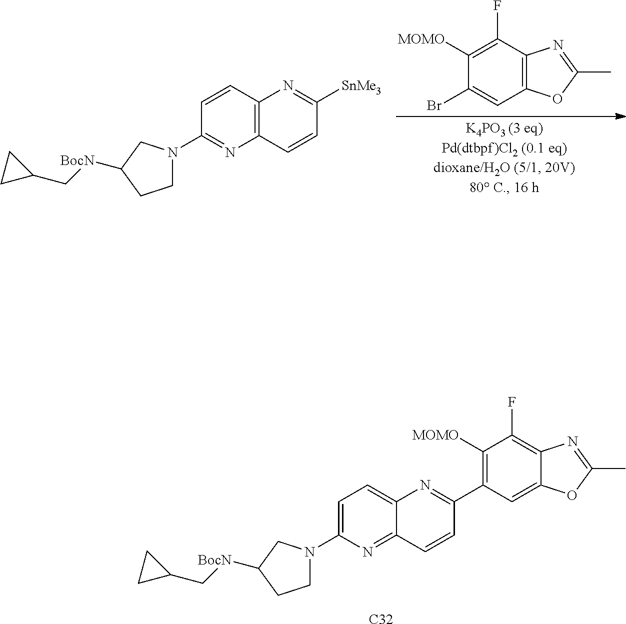

A mixture of tert-butyl N-(cyclopropylmethyl)-N-{1-[6-(trimethylstannyl)-1,5-naphthyridin-2-yl]pyrrolidin-3-yl}carbamate (600 mg, 1.129 mmol, 1 equiv), 6-bromo-4-fluoro-5-(methoxymethoxy)-2-methyl-1,3-benzoxazole (328 mg, 1.129 mmol, 1 equiv), Pd(DtBPF)Cl₂ (74 mg, 0.113 mmol, 0.1 equiv), and K₃PO₄ (719 mg, 3.387 mmol, 3 equiv) in dioxane (10 mL) and water (2 mL) was stirred for 16 hr at 80° C. under nitrogen atmosphere. The reaction mixture was cooled to room temperature, then poured into water (100 mL), extracted with ethyl acetate (2×100 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-(cyclopropylmethyl)-N-(1-{6-[4-fluoro-5-(methoxymethoxy)-2-methyl-1,3-benzoxazol-6-yl]-1,5-naphthyridin-2-yl}pyrrolidin-3-yl)carbamate (290 mg, 44%) as a solid. LCMS (ES, m/z):578 [M+H]⁺.

### Synthesis of Compound 294

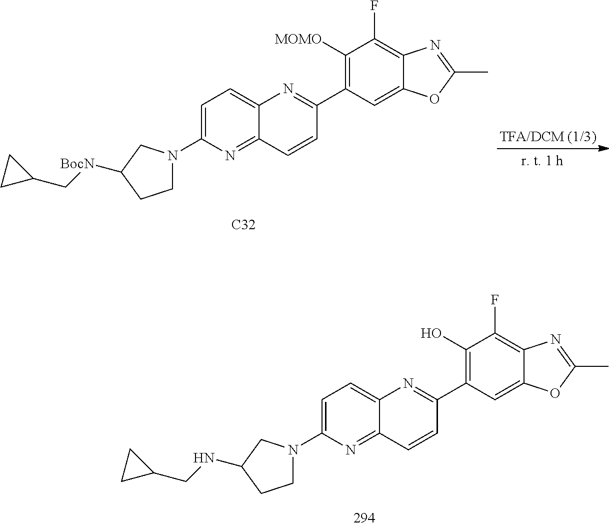

A solution of tert-butyl N-(cyclopropylmethyl)-N-(1-{6-[4-fluoro-5-(methoxymethoxy)-2-methyl-1,3-benzoxazol-6-yl]-1,5-naphthyridin-2-yl}pyrrolidin-3-yl)carbamate (290 mg, 0.502 mmol, 1 equiv) in TFA (1 mL) and DCM (3 mL) was stirred for 1 h at room temperature. The resulting mixture was concentrated under reduced pressure to give a residue. The residue was purified by prep-HPLC (Condition 3, Gradient 24) to afford 6-(6-{3-[(cyclopropylmethyl)amino]pyrrolidin-1-yl}-1,5-naphthyridin-2-yl)-4-fluoro-2-methyl-1,3-benzoxazol-5-ol (12.4 mg, 5%) as a solid. LCMS (ES, m/z):434 [M+H]⁺. ¹H NMR (400 MHz, DMSO-d₆) δ 15.08 (s, 1H), 8.45 (d, J=9.3 Hz, 1H), 8.33 (d, J=1.4 Hz, 1H), 8.17 (d, J=9.4 Hz, 1H), 8.11 (d, J=9.1 Hz, 1H), 7.19 (d, J=9.3 Hz, 1H), 3.74 (d, J=10.4 Hz, 1H), 3.69-3.58 (m, 1H), 3.58-3.47 (m, 2H), 3.47-3.40 (m, 1H), 2.64 (s, 3H), 2.47 (d, J=6.6 Hz, 2H), 2.14 (dd, J=12.6, 6.2 Hz, 1H), 1.93-1.89 (m, 1H), 0.95-0.84 (m, 1H), 0.46-0.37 (m, 2H), 0.18-0.10 (m, 2H).

### Synthesis of Compounds 323 and 324

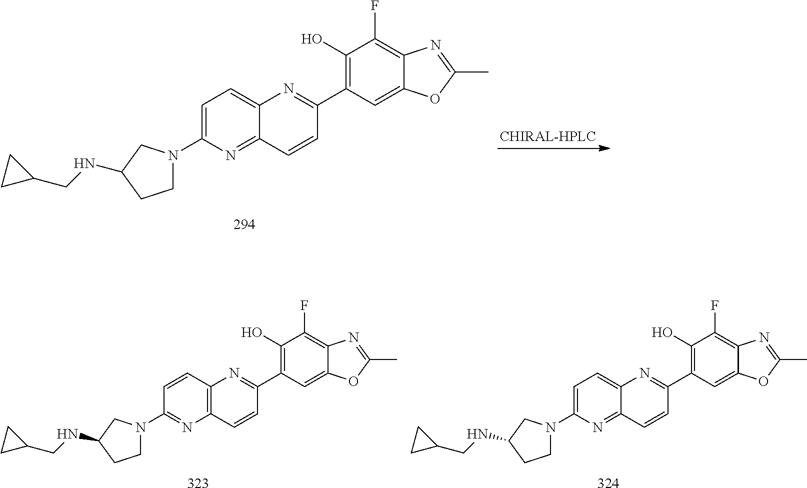 [SMILES: Cc1nc2c(F)c(O)c(-c3ccc4nc(N5CCC(NCC6CC6)C5)ccc4n3)cc2o1 ; "294" ; Cc1nc2c(F)c(O)c(-c3ccc4nc(N5CC[C@@H](NCC6CC6)C5)ccc4n3)cc2o1 ; Cc1nc2c(F)c(O)c(-c3ccc4nc(N5CC[C@H](NCC6CC6)C5)ccc4n3)cc2o1 ; "323" ; "324"]

6-(6-{3-[(cyclopropylmethyl)amino]pyrrolidin-1-yl}-1,5-naphthyridin-2-yl)-4-fluoro-2-methyl-1,3-benzoxazol-5-ol was purified by chiral reverse flash chromatography (Condition 1, Gradient 2) to afford (25.9 mg) and (25.9 mg) as solids. Compound 323: LCMS (ES, m/z):434 [M+H]⁺. ¹H NMR (400 MHz, DMSO-d₆) δ 15.07 (s, 1H), 8.43 (dd, J=9.3, 1.6 Hz, 1H), 8.32 (d, J=1.8 Hz, 1H), 8.16 (dd, J=9.4, 1.7 Hz, 1H), 8.11 (d, J=9.0 Hz, 1H), 7.18 (dd, J=9.4, 1.5 Hz, 1H), 3.79-3.66 (m, 2H), 3.58-3.47 (m, 2H), 3.47 (t, J=5.5 Hz, 1H), 2.64 (s, 3H), 2.47 (d, J=6.6 Hz, 2H), 2.14 (dq, J=12.9, 6.5 Hz, 1H), 1.93-1.85 (m, 1H), 0.95-0.84 (m, 1H), 0.46-0.36 (m, 2H), 0.18-0.10 (m, 2H). (second peak): LCMS (ES, m/z):434 [M+H]⁺. ¹H NMR (400 MHz, DMSO-d₆) δ 15.07 (s, 1H), 8.45 (d, J=9.2 Hz, 1H), 8.33 (d, J=1.5 Hz, 1H), 8.17 (d, J=9.3 Hz, 1H), 8.14-8.08 (m, 1H), 7.19 (d, J=9.3 Hz, 1H), 3.77-3.66 (m, 2H), 3.59-3.46 (m, 2H), 3.46-3.40 (d, J=6.5 Hz, 1H), 2.64 (s, 3H), 2.47 (d, J=6.5 Hz, 2H), 2.14 (dd, J=12.5, 6.4 Hz, 1H), 1.93-1.85 (m, 1H), 0.89 (dtd, J=12.3, 6.8, 5.8, 4.1 Hz, 1H), 0.46-0.37 (m, 2H), 0.18-0.10 (m, 2H).

### Example 116: Synthesis of Compound 325, 326, and 327

### Synthesis of Intermediate C33

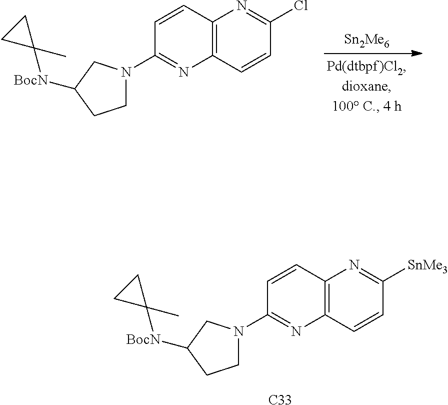

To a stirred mixture of tert-butyl N-[1-(6-chloro-1,5-naphthyridin-2-yl)pyrrolidin-3-yl]-N-(1-methylcyclopropyl)carbamate (500 mg, 1.241 mmol, 1 equiv) and Pd(DtBPF)Cl₂ (80.88 mg, 0.124 mmol, 0.1 equiv) in dioxane (15 mL) was added Sn₂Me₆ (609.85 mg, 1.862 mmol, 1.5 equiv) in portions at room temperature under nitrogen atmosphere. The resulting mixture was stirred for 4 hr at 100° C., then quenched with of a potassium fluoride solution (10 mL) at room temperature. The resulting mixture was extracted with ethyl acetate (2×50 mL). The organic layers were combined, washed with brine (2×50 mL), dried over anhydrous Na₂SO₄, and filtered. After filtration, the filtrate was concentrated under reduced pressure to afford tert-butyl N-(1-methylcyclopropyl)-N-{1-[6-(trimethylstannyl)-1,5-naphthyridin-2-yl]pyrrolidin-3-yl}carbamate (800 mg, 85%) as an oil.

### Synthesis of Intermediate C34

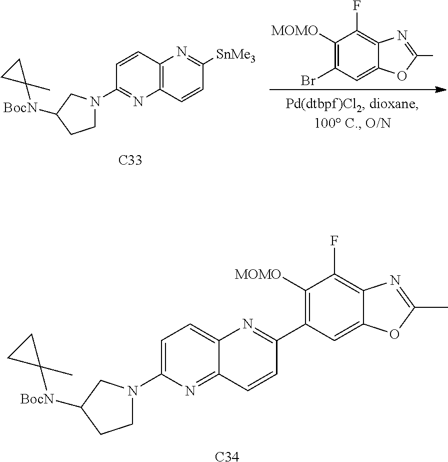

To a stirred mixture of tert-butyl N-(1-methylcyclopropyl)-N-{1-[6-(trimethylstannyl)-1,5-naphthyridin-2-yl]pyrrolidin-3-yl}carbamate (700 mg, 1.318 mmol, 1 equiv) and 6-bromo-4-fluoro-5-(methoxymethoxy)-2-methyl-1,3-benzoxazole (458.65 mg, 1.582 mmol, 1.2 equiv) in dioxane (35 mL) was added Pd(dtbpf)Cl₂ at room temperature under nitrogen atmosphere. The resulting mixture was stirred overnight at 100° C., then filtered. After filtration, the filtrate was concentrated under reduced pressure to give a residue. The residue was purified by silica gel column chromatography, eluted with CH₂Cl₂/MeOH (10:1) to afford tert-butyl N-(1-{6-[4-fluoro-5-(methoxymethoxy)-2-methyl-1,3-benzoxazol-6-yl]-1,5-naphthyridin-2-yl}pyrrolidin-3-yl)-N-(1-methylcyclopropyl)carbamate (350 mg, 46%) as a solid.

### Synthesis of Compound 325

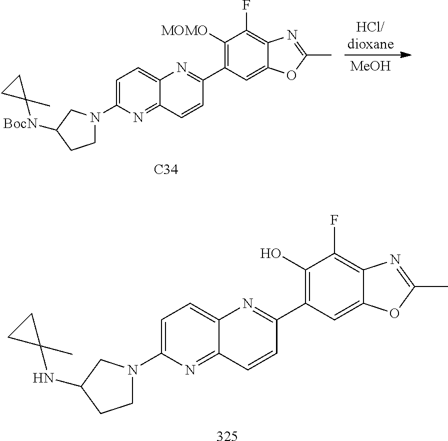

To a stirred solution of tert-butyl N-(1-{6-[4-fluoro-5-(methoxymethoxy)-2-methyl-1,3-benzoxazol-6-yl]-1,5-naphthyridin-2-yl}pyrrolidin-3-yl)-N-(1-methylcyclopropyl) carbamate (320 mg, 0.554 mmol, 1 equiv) in methanol (4 mL) was added HCl (gas) in 1,4-dioxane (4 mL) in portions at room temperature under nitrogen atmosphere. The resulting mixture was stirred for 2 hr at room temperature, then concentrated under reduced pressure to give a residue. The residue was purified by prep-HPLC (Condition 2, Gradient 12) to afford 4-fluoro-2-methyl-6-(6-{3-[(1-methylcyclopropyl)amino]pyrrolidin-1-yl}-1,5-naphthyridin-2-yl)-1,3-benzoxazol-5-ol (10.9 mg, 5%) as a solid. LCMS (ES, m/z): 434 [M+H]⁺. ¹H NMR (400 MHz, DMSO-d₆) δ 15.08 (s, 1H), 8.44 (d, J=9.2 Hz, 1H), 8.33 (d, J=1.4 Hz, 1H), 8.16-8.11 (d, J=9.4 Hz, 2H), 7.18 (d, J=9.3 Hz, 1H), 3.78 (s, 1H), 3.66 (s, 2H), 3.54 (d, J=9.0 Hz, 1H), 3.54 (d, 1H), 2.64 (s, 3H), 2.17-2.10 (m, 1H), 1.85 (s, 1H), 1.27 (s, 3H), 0.49 (q, J=10.9, 10.1 Hz, 2H), 0.35 (s, 2H).

### Synthesis of Compounds 326 and 327

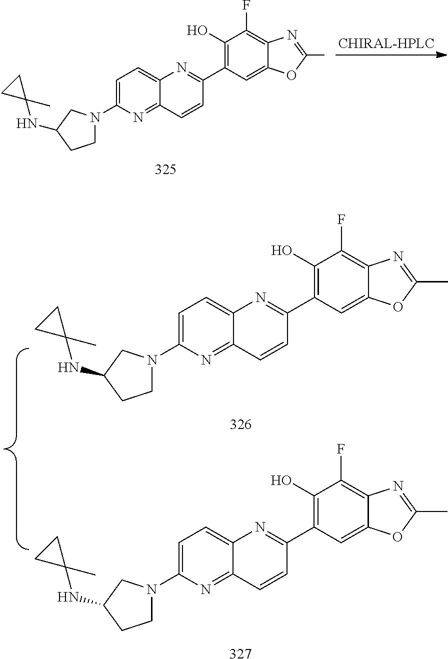 [SMILES: Cc1nc2c(F)c(O)c(-c3ccc4nc(N5CCC(NC6(C)CC6)C5)ccc4n3)cc2o1 ; "325" ; Cc1nc2c(F)c(O)c(-c3ccc4nc(N5CC[C@@H](NC6(C)CC6)C5)ccc4n3)cc2o1 ; "326" ; Cc1nc2c(F)c(O)c(-c3ccc4nc(N5CC[C@H](NC6(C)CC6)C5)ccc4n3)cc2o1 ; "327"]

4-fluoro-2-methyl-6-(6-{3-[(1-methylcyclopropyl)amino]pyrrolidin-1-yl}-1,5-naphthyridin-2-yl)-1,3-benzoxazol-5-ol was purified by chiral prep-HPLC (Condition 4, Gradient 3) to afford 4-fluoro-2-methyl-6-{6-[(3 S)-3-[(1-methylcyclopropyl)amino]pyrrolidin-1-yl]-1,5-naphthyridin-2-yl}-1,3-benzoxazol-5-ol (15.7 mg, 7%) and 4-fluoro-2-methyl-6-{6-[(3R)-3-[(1-methylcyclopropyl) amino]pyrrolidin-1-yl]-1,5-naphthyridin-2-yl}-1,3-benzoxazol-5-ol (15.8 mg, 7%) as solids. Compound 326: LCMS (ES, m/z): 434 [M+H]⁺. ¹H NMR (400 MHz, DMSO-d₆) δ 15.08 (s, 1H), 8.44 (d, J=9.2 Hz, 1H), 8.33 (d, J=1.4 Hz, 1H), 8.16-8.11 (d, J=9.4 Hz, 2H), 7.18 (d, J=9.3 Hz, 1H), 3.78 (s, 1H), 3.66 (s, 2H), 3.54 (d, J=9.0 Hz, 1H), 3.54 (d, 1H), 2.64 (s, 3H), 2.17-2.10 (m, 1H), 1.85 (s, 1H), 1.27 (s, 3H), 0.49 (q, J=10.9, 10.1 Hz, 2H), 0.35 (s, 2H). Compound 327: LCMS (ES, m/z): 434 [M+H]. ¹H NMR (400 MHz, DMSO-d₆) δ 15.08 (s, 1H), 8.44 (d, J=9.2 Hz, 1H), 8.33 (d, J=1.4 Hz, 1H), 8.16-8.11 (d, J=9.4 Hz, 2H), 7.18 (d, J=9.3 Hz, 1H), 3.78 (s, 1H), 3.66 (s, 2H), 3.54 (d, J=9.0 Hz, 1H), 3.54 (d, 1H), 2.64 (s, 3H), 2.17-2.10 (m, 1H), 1.85 (s, 1H), 1.27 (s, 3H), 0.49 (q, J=10.9, 10.1 Hz, 2H), 0.35 (s, 2H).

### Example 117: Synthesis of Compounds 309, 310, and 311

### Synthesis of Intermediate C35

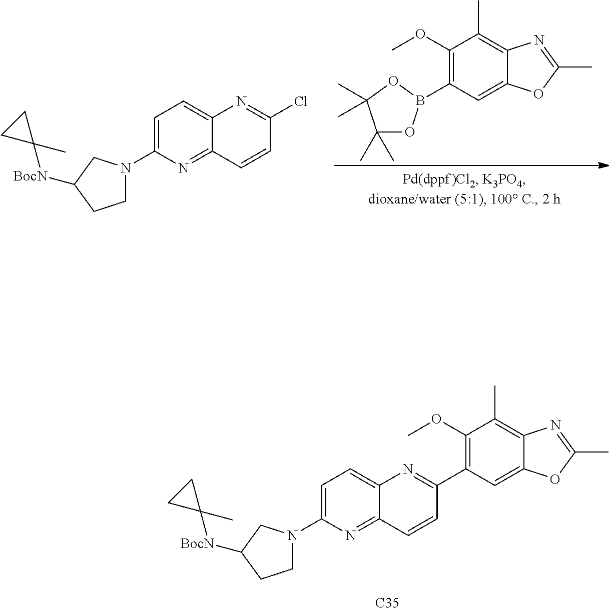

To a stirred mixture of tert-butyl N-[1-(6-chloro-1,5-naphthyridin-2-yl)pyrrolidin-3-yl]-N-(1-methylcyclopropyl)carbamate (500 mg, 1.241 mmol, 1 equiv), 5-methoxy-2,4-dimethyl-6-(4,4,5,5-tetramethyl-1,3,2-dioxaborolan-2-yl)-1,3-benzoxazole (395.03 mg, 1.303 mmol, 1.05 equiv), and Pd(dppf)Cl₂ (101.09 mg, 0.124 mmol, 0.1 equiv) in 1,4-dioxane (10 mL) was added K₃PO₄ (790.23 mg, 3.723 mmol, 3 equiv) in water (2 mL) dropwise at room temperature under nitrogen atmosphere. The resulting mixture was stirred for 2 hr at 100° C. under nitrogen atmosphere. The resulting mixture was cooled to room temperature, then diluted with water (30 mL) and extracted with CH₂Cl₂ (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (2:1) to afford tert-butyl N-{1-[6-(5-methoxy-2,4-dimethyl-1,3-benzoxazol-6-yl)-1,5-naphthyridin-2-yl]pyrrolidin-3-yl}-N-(1-methylcyclopropyl)carbamate (600 mg, 89%) as a solid. LCMS (ESI, m/z): 544[M+H].

### Synthesis of Compound 309

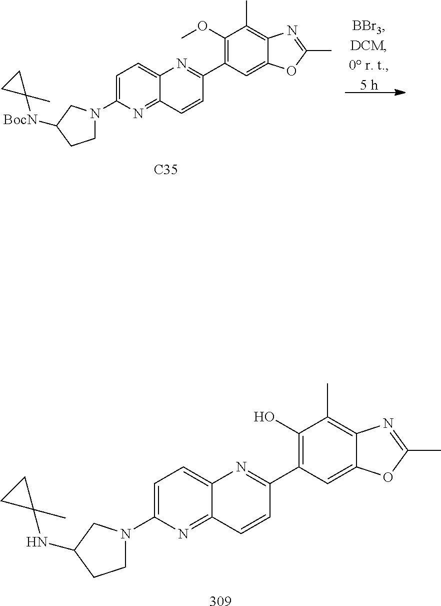 [SMILES: COc1c(-c2ccc3nc(N4CCC(N(C(=O)OC(C)(C)C)C5(C)CC5)C4)ccc3n2)cc2oc(C)nc2c1C ; "BBr3,DCM,0° r. t.,5 h" ; "C35" ; Cc1nc2c(C)c(O)c(-c3ccc4nc(N5CCC(NC6(C)CC6)C5)ccc4n3)cc2o1 ; "309"]

To a stirred solution of tert-butyl N-{1-[6-(5-methoxy-2,4-dimethyl-1,3-benzoxazol-6-yl)-1,5-naphthyridin-2-yl]pyrrolidin-3-yl}-N-(1-methylcyclopropyl)carbamate (600 mg, 1.104 mmol, 1 equiv) in DCM (30 mL) was added BBr₃ (2764.81 mg, 11.040 mmol, 10 equiv) dropwise at 0° C. under nitrogen atmosphere. The resulting mixture was stirred for 5 hr at room temperature under nitrogen atmosphere, then basified to pH 7 with NH₃ in methanol (7 M) at 0° C., diluted with water (20 mL) and extracted with CH₂Cl₂ (3×20 mL). The organic layers were combined, washed with brine (1×20 mL), dried over anhydrous Na₂SO₄, and filtered. After filtration, the filtrate was concentrated under reduced pressure to give a residue. The residue was dissolved in methanol (3 mL). A precipitate formed that was collected by filtration and washed with methanol (3×1 mL) to afford 2,4-dimethyl-6-(6-{3-[(1-methylcyclopropyl)amino]pyrrolidin-1-yl}-1,5-naphthyridin-2-yl)-1,3-benzoxazol-5-ol (200 mg) as a solid. The solid was purified by prep-HPLC (Condition 1, Gradient 27) to afford 2,4-dimethyl-6-(6-{3-[(1-methylcyclopropyl)amino]pyrrolidin-1-yl}-1,5-naphthyridin-2-yl)-1,3-benzoxazol-5-ol (100 mg) as a solid. LCMS (ESI, m/z): 430[M+H]. ¹H NMR (400 MHz, DMSO-d₆): δ 14.99 (s, 1H), 8.40 (d, J=9.3 Hz, 1H), 8.24 (s, 1H), 8.09 (dd, J=22.2, 9.2 Hz, 2H), 7.14 (d, J=9.3 Hz, 1H), δ 3.77 (s, 1H), 3.64 (t, J=5.7 Hz, 2H), 3.52 (d, J=9.7 Hz, 1H), 3.31 (s, 1H), 2.61 (s, 3H), 2.40 (s, 3H), 2.34 (s, 1H), 2.13 (dd, J=12.1, 6.3 Hz, 1H), 1.88 (d, J=10.6 Hz, 1H), 1.26 (s, 3H), 0.47 (qd, J=11.7, 10.6, 2.4 Hz, 2H), 0.38-0.28 (m, 2H).

### Synthesis of Compounds 310 and 311

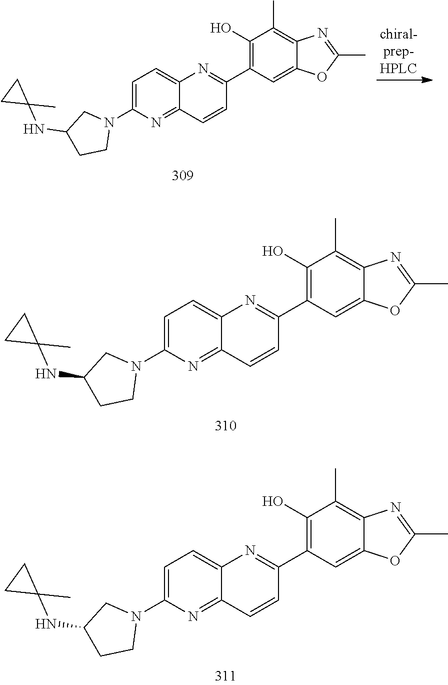 [SMILES: Cc1nc2c(C)c(O)c(-c3ccc4nc(N5CCC(NC6(C)CC6)C5)ccc4n3)cc2o1 ; Cc1nc2c(C)c(O)c(-c3ccc4nc(N5CC[C@@H](NC6(C)CC6)C5)ccc4n3)cc2o1 ; Cc1nc2c(C)c(O)c(-c3ccc4nc(N5CC[C@H](NC6(C)CC6)C5)ccc4n3)cc2o1]

2,4-dimethyl-6-(6-{3-[(1-methylcyclopropyl)amino]pyrrolidin-1-yl}-1,5-naphthyridin-2-yl)-1,3-benzoxazol-5-ol (185 mg, 0.414 mmol) was purified by chiral prep-HPLC (Condition 4, Gradient 1) to afford 2,4-dimethyl-6-{6-[(3R)-3-[(1-methylcyclopropyl)amino]pyrrolidin-1-yl]-1,5-naphthyridin-2-yl}-1,3-benzoxazol-5-ol (67.8 mg, 38%, first peak) and 2,4-dimethyl-6-{6-[(3S)-3-[(1-methylcyclopropyl)amino]pyrrolidin-1-yl]-1,5-naphthyridin-2-yl}-1,3-benzoxazol-5-ol (68.6 mg, 38.05%, second peak) as solids. Compound 310: LCMS (ESI, m/z): 430 [M+H]. ¹H NMR (400 MHz, DMSO-d₆): δ 14.99 (s, 1H), 8.39 (d, J=9.3 Hz, 1H), 8.23 (s, 1H), 8.09 (dd, J=21.9, 9.2 Hz, 2H), 7.13 (d, J=9.3 Hz, 1H), 3.76 (s, 1H), 3.67-3.60 (m, 2H), 3.53 (s, 1H), 3.30 (s, 1H), 2.61 (s, 3H), 2.40 (s, 3H), 2.13 (dq, J=12.9, 6.3 Hz, 1H), 1.95-1.77 (m, 1H), 1.26 (s, 3H), 0.48 (tt, J=11.0, 6.0 Hz, 2H), 0.33 (d, J=3.2 Hz, 2H). Compound 311: LCMS (ESI, m/z): 430 [M+H]. ¹H NMR (400 MHz, DMSO-d₆): δ 14.99 (s, 1H), 8.40 (d, J=9.2 Hz, 1H), 8.24 (s, 1H), 8.12 (d, J=9.2 Hz, 1H), 8.07 (d, J=9.1 Hz, 1H), 7.14 (d, J=9.3 Hz, 1H), 3.77 (s, 1H), 3.68-3.60 (m, 2H), 3.52 (d, J=9.5 Hz, 1H), 3.30 (s, 1H), 2.61 (s, 3H), 2.40 (s, 3H), 2.19-2.08 (m, 1H), 1.88 (s, 1H), 1.26 (s, 3H), 0.54-0.41 (m, 2H), 0.33 (d, J=3.3 Hz, 2H).

### Example 118: Synthesis of Compounds 295 and 296

### Synthesis of Intermediate C36

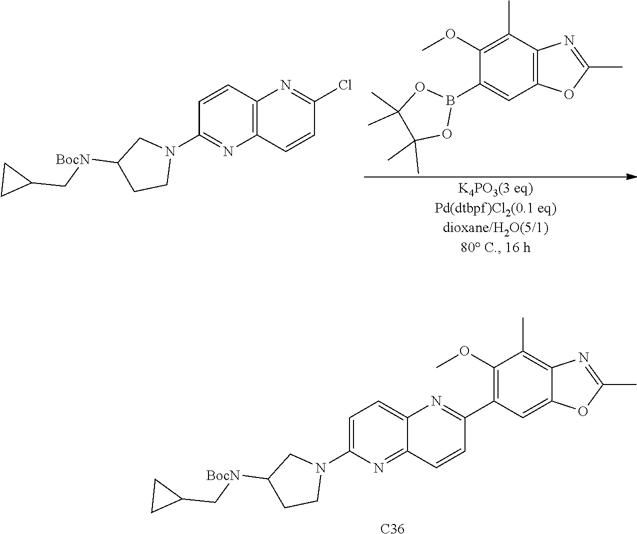

A mixture of tert-butyl N-[1-(6-chloro-1,5-naphthyridin-2-yl)pyrrolidin-3-yl]-N-(cyclopropylmethyl)carbamate (500 mg, 1.241 mmol, 1 equiv), 5-methoxy-2,4-dimethyl-6-(4,4,5,5-tetramethyl-1,3,2-dioxaborolan-2-yl)-1,3-benzoxazole (376 mg, 1.241 mmol, 1 equiv), Pd(DtBPF)Cl₂ (81 mg, 0.124 mmol, 0.1 equiv), and K₃PO₄ (790 mg, 3.723 mmol, 3 equiv) in dioxane (10 mL) and water (2 mL) was stirred for 16 hr at 80° C. under nitrogen atmosphere. The reaction mixture was cooled to room temperature, then poured into water (20 mL) and extracted with ethyl acetate (3×20 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-(cyclopropylmethyl)-N-{1-[6-(5-methoxy-2,4-dimethyl-1,3-benzoxazol-6-yl)-1,5-naphthyridin-2-yl]pyrrolidin-3-yl}carbamate (360 mg, 53%) as a solid. LCMS (ES, m/z):544 [M+H]⁺.

### Synthesis of Intermediate C37

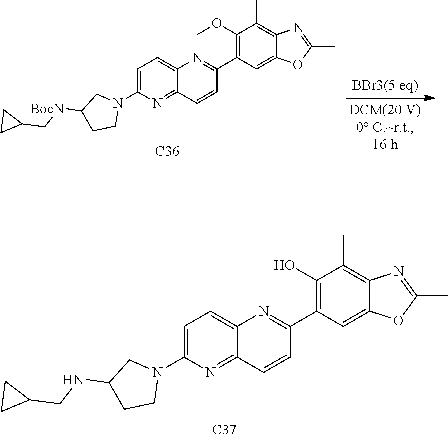 [SMILES: COc1c(-c2ccc3nc(N4CCC(N(CC5CC5)C(=O)OC(C)(C)C)C4)ccc3n2)cc2oc(C)nc2c1C ; "C36" ; Cc1nc2c(C)c(O)c(-c3ccc4nc(N5CCC(NCC6CC6)C5)ccc4n3)cc2o1 ; "C37"]

To a stirred solution of tert-butyl N-(cyclopropylmethyl)-N-{1-[6-(5-methoxy-2,4-dimethyl-1,3-benzoxazol-6-yl)-1,5-naphthyridin-2-yl]pyrrolidin-3-yl}carbamate (360 mg, 0.662 mmol, 1 equiv) in DCM (7.2 mL) was added BBr₃ (829 mg, 3.310 mmol, 5.00 equiv) dropwise at 0° C. The resulting mixture was stirred for 16 hr at room temperature, then quenched with methanol (30 mL) at 0° C., basified to pH 8 with saturated NaHCO₃ (aq.), and extracted with CH₂Cl₂ (3×50 mL), dried over anhydrous Na₂SO₄, and filtered. After filtration, the filtrate was concentrated under reduced pressure to give a residue. The residue was purified by chiral prep-HPLC (Condition 3, Gradient 22) to afford 6-(6-{3-[(cyclopropylmethyl)amino]pyrrolidin-1-yl}-1,5-naphthyridin-2-yl)-2,4-dimethyl-1,3-benzoxazol-5-ol (140 mg) as a solid. LCMS (ES, m/z):430 [M+H]⁺. ¹H NMR (400 MHz, DMSO-d₆) δ 14.99 (s, 1H), 8.41 (d, J=9.3 Hz, 1H), 8.25 (s, 1H), 8.14 (dd, J=9.2, 0.8 Hz, 1H), 8.07 (dd, J=9.1, 0.8 Hz, 1H), 7.16 (d, J=9.3 Hz, 1H), 3.74 (dd, J=10.5, 5.8 Hz, 1H), 3.68 (s, 1H), 3.60-3.51 (m, 1H), 3.45 (t, J=5.4 Hz, 1H), 3.41-3.30 (s, 1H), 2.61 (s, 3H), 2.46 (d, J=6.8 Hz, 2H), 2.40 (s, 3H), 2.13 (dq, J=12.9, 6.5 Hz, 1H), 1.87 (dd, J=12.4, 6.4 Hz, 1H), 0.95-0.84 (m, 1H), 0.46-0.33 (m, 2H), 0.17-0.09 (m, 2H).

### Synthesis of Compounds 295 and 296

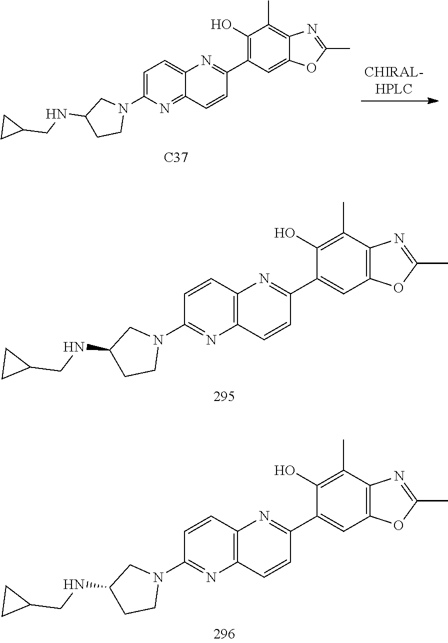 [SMILES: Cc1nc2c(C)c(O)c(-c3ccc4nc(N5CCC(NCC6CC6)C5)ccc4n3)cc2o1 ; "C37" ; Cc1nc2c(C)c(O)c(-c3ccc4nc(N5CC[C@@H](NCC6CC6)C5)ccc4n3)cc2o1 ; Cc1nc2c(C)c(O)c(-c3ccc4nc(N5CC[C@H](NCC6CC6)C5)ccc4n3)cc2o1]

6-(6-{3-[(cyclopropylmethyl)amino]pyrrolidin-1-yl}-1,5-naphthyridin-2-yl)-2,4-dimethyl-1,3-benzoxazol-5-ol (120 mg, 0.279 mmol, 1 equiv) was purified by chiral prep-HPLC (Column: CHIRAL ART Cellulose-SB, 2×25 cm, 5 um; Mobile Phase A: MtBE(0.1% DEA)-HPLC, Mobile Phase B: MeOH-HPLC; Flow rate: 20 mL/min; Gradient: 20% B to 20% B in 7 min; Wave Length: UV 220/254 nm; RT1(min): 5.6; RT2(min)) to afford Compound 295 (40.2 mg) and Compound 296 (36.7 mg) as solids. Compound 295: LCMS (ES, m/z):430 [M+H]⁺. ¹H NMR (400 MHz, DMSO-d₆) δ 14.99 (s, 1H), 8.41 (d, J=9.2 Hz, 1H), 8.25 (s, 1H), 8.15 (d, J=9.2 Hz, 1H), 8.08 (d, J=9.2 Hz, 1H), 7.17 (d, J=9.3 Hz, 1H), 3.74 (dd, J=10.8, 5.8 Hz, 1H), 3.68 (s, 1H), 3.58 (s, 1H), 3.46 (t, J=5.6 Hz, 1H), 3.46-3.32 (m, 1H), 2.61 (s, 3H), 2.47 (d, J=6.7 Hz, 2H), 2.40 (s, 3H), 2.14 (dq, J=12.7, 6.5 Hz, 1H), 1.92-1.84 (m, 1H), 0.94-0.84 (m, 1H), 0.46-0.35 (m, 2H), 0.17-0.09 (m, 2H). Compound 296: LCMS (ES, m/z):430 [M+H]⁺. ¹H NMR (400 MHz, DMSO-d₆) δ 15.00 (s, 1H), 8.42 (d, J=9.3 Hz, 1H), 8.26 (s, 1H), 8.15 (d, J=9.3 Hz, 1H), 8.08 (d, J=9.0 Hz, 1H), 7.17 (d, J=9.3 Hz, 1H), 3.75 (dd, J=10.8, 5.8 Hz, 1H), 3.68 (s, 1H), 3.58 (s, 1H), 3.46 (t, J=5.7 Hz, 1H), 3.46-3.30 (m, 1H), 2.47 (d, J=6.1 Hz, 5H), 2.40 (s, 3H), 2.13 (dt, J=13.0, 6.4 Hz, 1H), 1.92-1.84 (m, 1H), 0.96-0.84 (m, 1H), 0.46-0.37 (m, 2H), 0.18-0.10 (m, 2H).

### Example 119: Synthesis of Compound 297

### Synthesis of Intermediate C38

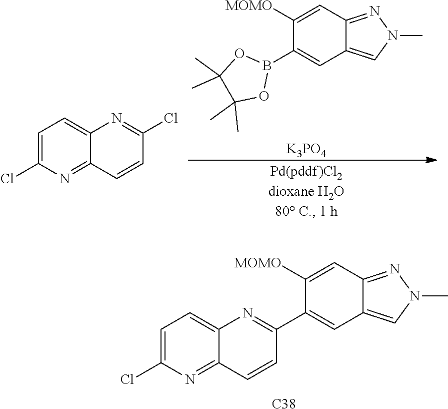

To a stirred mixture of 2,6-dichloro-1,5-naphthyridine (300 mg, 1.507 mmol, 1 equiv) and 6-(methoxymethoxy)-2-methyl-5-(4,4,5,5-tetramethyl-1,3,2-dioxaborolan-2-yl) indazole (479.60 mg, 1.507 mmol, 1 equiv) in dioxane (6 mL) and water (1.2 mL) was added Pd(dppf)Cl₂·CH₂Cl₂ (122.79 mg, 0.151 mmol, 0.1 equiv) and K₃PO₄ (959.85 mg, 4.521 mmol, 3 equiv) at room temperature under nitrogen atmosphere. The mixture was stirred at 80° C. for 1 hr. The resulting mixture was concentrated under vacuum to give a residue. The residue was purified by silica gel column chromatography, eluted with PE/EA (1:1) to afford 2-chloro-6-[6-(methoxymethoxy)-2-methylindazol-5-yl]-1,5-naphthyridine (260 mg, 49%) as a solid.

### Synthesis of Intermediate C39

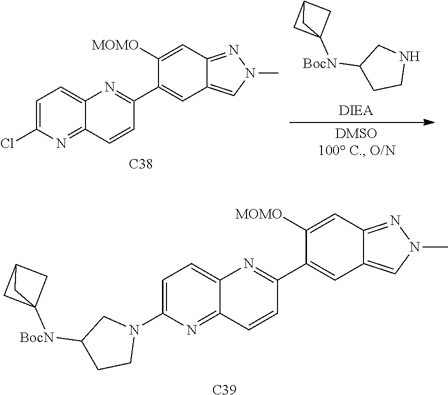

To a stirred mixture of 2-chloro-6-[6-(methoxymethoxy)-2-methylindazol-5-yl]-1,5-naphthyridine (100 mg, 0.282 mmol, 1 equiv) and tert-butyl N-{bicyclo[1.1.1]pentan-1-yl}-N-(pyrrolidin-3-yl)carbamate (85.35 mg, 0.338 mmol, 1.2 equiv) in DMSO (3 mL) was added DIEA (109.28 mg, 0.846 mmol, 3 equiv) dropwise at room temperature under nitrogen atmosphere. The resulting mixture was stirred at 100° C. overnight, then extracted with ethyl acetate (2×20 mL). The organic layers were combined, washed with water (2×20 mL), dried over anhydrous Na₂SO₄, and filtered. After filtration, the filtrate was concentrated under reduced pressure to give tert-butyl N-{bicyclo[1.1.1]pentan-1-yl}-N-(1-{6-[6-(methoxymethoxy)-2-methylindazol-5-yl]-1,5-naphthyridin-2-yl}pyrrolidin-3-yl)carbamate (150 mg) as a solid.

### Synthesis of Compound 297

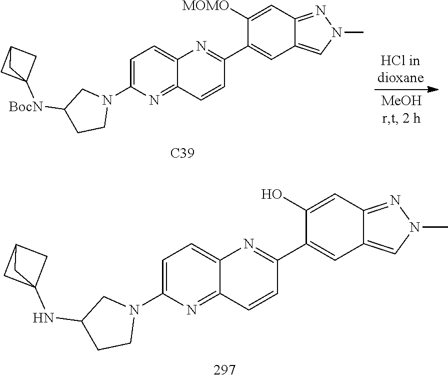

To a stirred mixture of tert-butyl N-{bicyclo[1.1.1]pentan-1-yl}-N-(1-{6-[6-(methoxymethoxy)-2-methylindazol-5-yl]-1,5-naphthyridin-2-yl}pyrrolidin-3-yl)carbamate (150 mg, 0.263 mmol, 1 equiv) in methanol (1.5 mL, 37.048 mmol, 140.96 equiv) was added HCl (gas) in 1,4-dioxane (1.5 mL, 49.368 mmol, 187.83 equiv) dropwise at room temperature under nitrogen atmosphere. The resulting mixture was stirred at room temperature for 2 hr, then concentrated under reduced pressure to give a residue. The residue product was purified by reverse phase (Condition 5, Gradient 1) to afford 5-[6-(3-{bicyclo[1.1.1]pentan-1-ylamino}pyrrolidin-1-yl)-1,5-naphthyridin-2-yl]-2-methylindazol-6-ol (47.9 mg, 43%) as a solid. LCMS (ES, m/z): 427 [M+H]⁺. ¹H NMR (400 MHz, DMSO-d₆) δ 13.97 (s, 1H), 8.54 (s, 1H), 8.39 (d, J=9.2 Hz, 1H), 8.35 (s, 1H), 8.08 (dd, J=12.9, 9.2 Hz, 2H), 7.14 (d, J=9.3 Hz, 1H), 6.87 (s, 1H), 4.11 (s, 3H), 3.78 (dd, J=10.6, 6.2 Hz, 1H), 3.68 (s, 1H), 3.55 (t, J=8.3 Hz, 1H), 3.47 (s, 1H), 3.32 (s, 1H), 2.77 (s, 1H), 2.37 (s, 1H), 2.16 (dq, J=12.6, 6.2 Hz, 1H), 1.78 (d, J=1.7 Hz, 7H).

### Example 120: Synthesis of Compound 333

### Synthesis of Intermediate C40

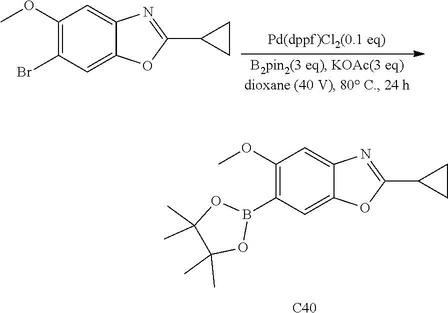 [SMILES: COc1cc2nc(C3CC3)oc2cc1Br ; COc1cc2nc(C3CC3)oc2cc1B1OC(C)(C)C(C)(C)O1 ; "C40"]

A mixture of 6-bromo-2-cyclopropyl-5-methoxy-1,3-benzoxazole (200 mg, 0.746 mmol, 1 equiv), 4,4,5,5-tetramethyl-2-(tetramethyl-1,3,2-dioxaborolan-2-yl)-1,3,2-dioxaborolane (568 mg, 2.238 mmol, 3 equiv), Pd(dppf)Cl₂ (55 mg, 0.075 mmol, 0.1 equiv), and KOAc (220 mg, 2.238 mmol, 3 equiv) in 1,4-dioxane (8 mL) was stirred for 24 hr at 80° C. under nitrogen atmosphere. The resulting mixture was cooled to room temperature, then poured into the water (50 mL) and extracted with ethyl acetate (1×40 mL), dried over anhydrous Na₂SO₄, and filtered. After filtration, the filtrate was concentrated under reduced pressure to afford 2-cyclopropyl-5-methoxy-6-(4,4,5,5-tetramethyl-1,3,2-dioxaborolan-2-yl)-1,3-benzoxazole (589 mg) as a solid. LCMS (ES, m/z): 316 [M+H]⁺.

### Synthesis of Intermediate C41

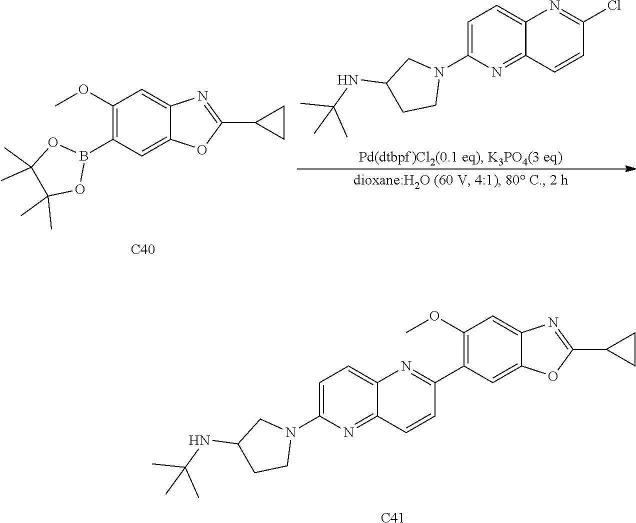

A mixture of N-tert-butyl-1-(6-chloro-1,5-naphthyridin-2-yl)pyrrolidin-3-amine (175 mg, 0.574 mmol, 1 equiv), 2-cyclopropyl-5-methoxy-6-(4,4,5,5-tetramethyl-1,3,2-dioxaborolan-2-yl)-1,3-benzoxazole (452.37 mg, 1.435 mmol, 2.5 equiv), Pd(DtBPF)Cl₂ (37 mg, 0.057 mmol, 0.1 equiv), and K₃PO₄ (366 mg, 1.722 mmol, 3 equiv) in 1,4-dioxane (8.4 mL) and water (2.1 mL) was stirred for 2 hr at 80° C. under nitrogen atmosphere. The resulting mixture was cooled to room temperature and extracted with ethyl acetate (1×50 mL). The organic layers were combined, dried over anhydrous Na₂SO₄, and filtered. After filtration, the filtrate was concentrated under reduced pressure to give a residue. The residue was purified by silica gel column chromatography, eluted with CH₂Cl₂/MeOH (10:1) to afford N-tert-butyl-1-[6-(2-cyclopropyl-5-methoxy-1,3-benzoxazol-6-yl)-1,5-naphthyridin-2-yl]pyrrolidin-3-amine (65 mg, 25%) as a solid. LCMS (ES, m/z):458 [M+H]⁺.

### Synthesis of Compound 333

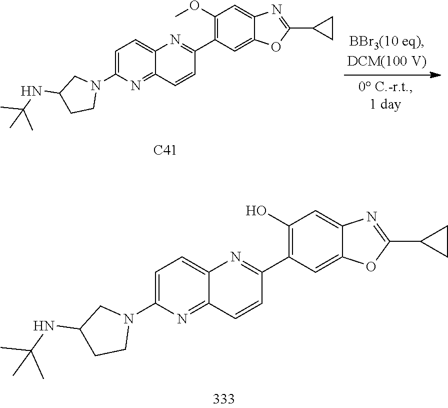 [SMILES: COc1cc2nc(C3CC3)oc2cc1-c1ccc2nc(N3CCC(NC(C)(C)C)C3)ccc2n1 ; "C41" ; CC(C)(C)NC1CCN(c2ccc3nc(-c4cc5oc(C6CC6)nc5cc4O)ccc3n2)C1]

To a stirred solution of N-tert-butyl-1-[6-(2-cyclopropyl-5-methoxy-1,3-benzoxazol-6-yl)-1,5-naphthyridin-2-yl]pyrrolidin-3-amine (65 mg, 0.142 mmol, 1 equiv) in DCM (6.5 mL) was added BBr₃ (355.87 mg, 1.420 mmol, 10 equiv) dropwise at 0° C. under nitrogen atmosphere. The resulting mixture was quenched with methanol (25 mL) at 0° C., then neutralized to pH 9 with saturated NaHCO₃ (aq.). The resulting mixture was extracted with CH₂Cl₂ (1×10 mL). The origin layers were combined, dried over anhydrous Na₂SO₄, and filtered. After filtration, the filtrate was concentrated under reduced pressure to give a residue. The residue was purified by Prep-HPLC (Column: XBridge Prep OBD C18 Column, 30×150 mm, 5 m; Mobile Phase A: Water (10 mmol/L NH₄HCO₃), Mobile Phase B: ACN; Flow rate: 60 mL/min; Gradient: 40% B to 70% B in 8 min, 70% B; Wave Length: UV 220 nm; RT1(min): 7.62)) to afford 6-{6-[3-(tert-butylamino)pyrrolidin-1-yl]-1,5-naphthyridin-2-yl}-2-cyclopropyl-1,3-benzoxazol-5-ol (8.9 mg, 14%) as a solid. LCMS (ES, m/z):444 [M+H]⁺. ¹H NMR (400 MHz, DMSO-d₆) δ 14.54 (s, 1H), 8.43-8.35 (m, 2H), 8.14-8.04 (m, 2H), 7.15 (d, J=9.3 Hz, 1H), 7.06 (s, 1H), 3.86 (s, 1H), 3.72 (s, 1H), 3.50 (s, 2H), 3.14 (s, 1H), 2.32-2.22 (m, 1H), 2.19 (s, 1H), 1.77 (s, 2H), 1.21 (m, 2H), 1.16 (m, 2H), 1.10 (s, 9H).

### Example 121: Synthesis of Compound 453

### Synthesis of Compound C42

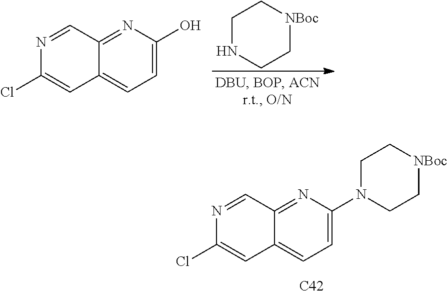 [SMILES: Oc1ccc2cc(Cl)ncc2n1 ; CC(C)(C)OC(=O)N1CCNCC1 ; CC(C)(C)OC(=O)N1CCN(c2ccc3cc(Cl)ncc3n2)CC1 ; "C42"]

To a stirred mixture of 6-chloro-1,7-naphthyridin-2-ol (200 mg, 1.107 mmol, 1 equiv) and tert-butyl piperazine-1-carboxylate (309.41 mg, 1.660 mmol, 1.5 equiv) in acetonitrile (4 mL) was added DBU (505.81 mg, 3.321 mmol, 3 equiv) and BOP (979.65 mg, 2.214 mmol, 2 equiv) dropwise at room temperature under nitrogen atmosphere. The resulting mixture was stirred overnight at room temperature. A precipitate formed that was collected by filtration, then washed with acetonitrile (2×2 mL) to afford tert-butyl 4-(6-chloro-1,7-naphthyridin-2-yl)piperazine-1-carboxylate (70 mg, 18%) as a solid.

### Synthesis of Intermediate C43

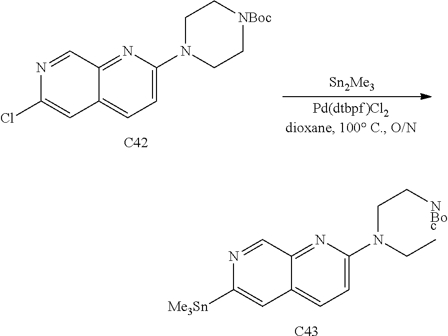

To a stirred mixture of tert-butyl 4-(6-chloro-1,7-naphthyridin-2-yl)piperazine-1-carboxylate (60 mg, 0.172 mmol, 1 equiv) and Pd(dtbpf)Cl₂ (11.21 mg, 0.017 mmol, 0.1 equiv) in dioxane (2 mL) was added Sn₂Me₆ (112.71 mg, 0.344 mmol, 2 equiv) dropwise at room temperature under nitrogen atmosphere. The resulting mixture was stirred overnight at 100° C., then quenched with KF at room temperature. The resulting mixture was filtered and the filter cake was washed with ethyl acetate (2×5 mL). The filtrate was concentrated under reduced pressure to give a residue. The residue was partitioned between water and ethyl acetate, then extracted with ethyl acetate (2×5 mL). The organic layers were combined, washed with brine (2×10 mL), dried over anhydrous Na₂SO₄, and filtered. After filtration, the filtrate was concentrated under reduced pressure to give a residue.

### Synthesis of Intermediate C44

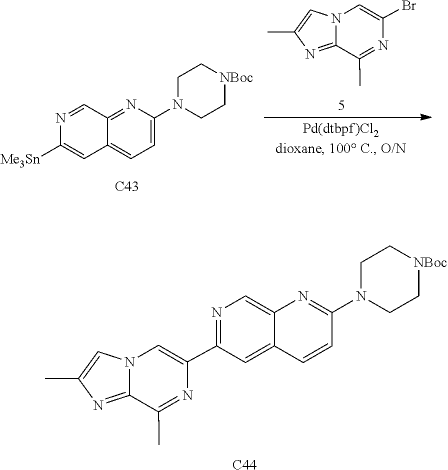

To a stirred mixture of 6-bromo-2,8-dimethylimidazo[1,2-a]pyrazine (78.17 mg, 0.347 mmol, 1.5 equiv) and Pd(dtbpf)Cl₂ (15.02 mg, 0.023 mmol, 0.1 equiv) in dioxane (3 mL) was added tert-butyl 4-[6-(trimethylstannyl)-1,7-naphthyridin-2-yl]piperazine-1-carboxylate (110 mg, 0.231 mmol, 1 equiv) dropwise at room temperature under nitrogen atmosphere. The resulting mixture was stirred overnight at 100° C., then quenched with water at room temperature and extracted with ethyl acetate (2×10 mL). The organic layers were combined, washed with brine (2×10 mL), dried over anhydrous Na₂SO₄, and filtered. After filtration, the filtrate was concentrated under reduced pressure to give a residue. The residue was purified by Prep-TLC (CH₂Cl₂/MeOH 10:1) to afford tert-butyl 4-(6-{2,8-dimethylimidazo [1,2-a]pyrazin-6-yl}-1,7-naphthyridin-2-yl)piperazine-1-carboxylate (100 mg, 28%) as a solid.

### Synthesis of Compound 453

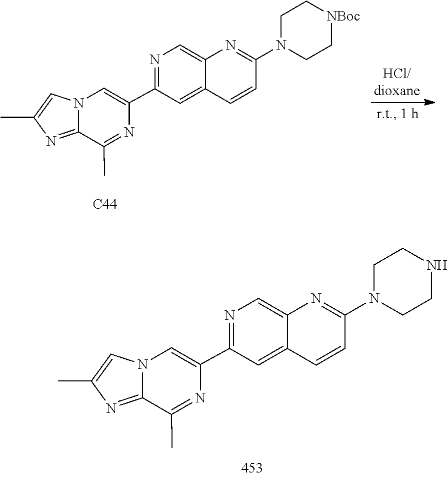 [SMILES: Cc1cn2cc(-c3cc4ccc(N5CCN(C(=O)OC(C)(C)C)CC5)nc4cn3)nc(C)c2n1 ; "C44" ; Cc1cn2cc(-c3cc4ccc(N5CCNCC5)nc4cn3)nc(C)c2n1]

To a stirred solution of tert-butyl 4-(6-{2,8-dimethylimidazo[1,2-a]pyrazin-6-yl}-1,7-naphthyridin-2-yl)piperazine-1-carboxylate (30 mg, 0.065 mmol, 1 equiv) in methanol (1 mL) was added HCl (gas) in 1,4-dioxane (1 mL) dropwise at room temperature under nitrogen atmosphere. The resulting mixture was stirred for 1 h at room temperature, then concentrated under reduced pressure to give a residue. The residue was purified by reverse phase chromatography (Gradient: isocratic) to afford 6-{2,8-dimethylimidazo[1,2-a]pyrazin-6-yl}-2-(piperazin-1-yl)-1,7-naphthyridine (1.2 mg, 5%) as a solid. LCMS (ES, m/z): 360 [M+H]⁺. ¹H NMR (400 MHz, DMSO-d₆) δ 9.47 (s, 1H), 9.21 (s, 2H), 9.05 (s, 1H), 8.61 (s, 1H), 8.39 (d, J=9.3 Hz, 1H), 8.18 (s, 1H), 7.62 (d, J=9.3 Hz, 1H), 4.03 (t, J=5.3 Hz, 4H), 3.25 (s, 4H), 2.89 (s, 3H), 2.48 (s, 3H).

### Example 122: Synthesis of Compounds 358 and 359

### Synthesis of Intermediate C45

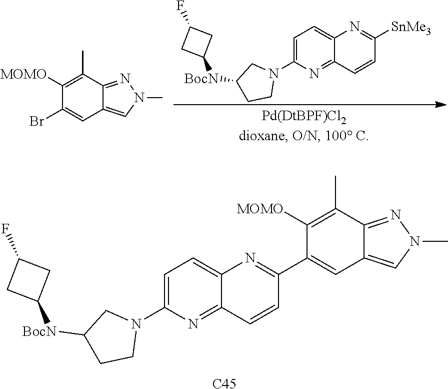

To a stirred mixture of 5-bromo-6-(methoxymethoxy)-2,7-dimethylindazole (100 mg, 0.35 mmol, 1 equiv) and tert-butyl N-[(1r,3r)-3-fluorocyclobutyl]-N-[(3S)-1-[6-(trimethylstannyl)-1,5-naphthyridin-2-yl]pyrrolidin-3-yl]carbamate (288.95 mg, 0.52 mmol, 1.5 equiv) in dioxane (3 mL) was added Pd(DtBPF)Cl₂ (22.86 mg, 0.03 mmol, 0.1 equiv) dropwise at room temperature under nitrogen atmosphere. The resulting mixture was stirred overnight at 100° C., then filtered. After filtration, the filtrate was concentrated under reduced pressure to give a residue. The residue was purified by silica gel column chromatography, eluted with CH₂Cl₂/MeOH (10:1) to afford tert-butyl N-(1-{6-[6-(methoxymethoxy)-2,7-dimethylindazol-5-yl]-1,5-naphthyridin-2-yl}pyrrolidin-3-yl)-N-[(1r,3r)-3-fluorocyclobutyl]carbamate (80 mg, 39%) as a solid.

### Synthesis of Compound 358 and 359

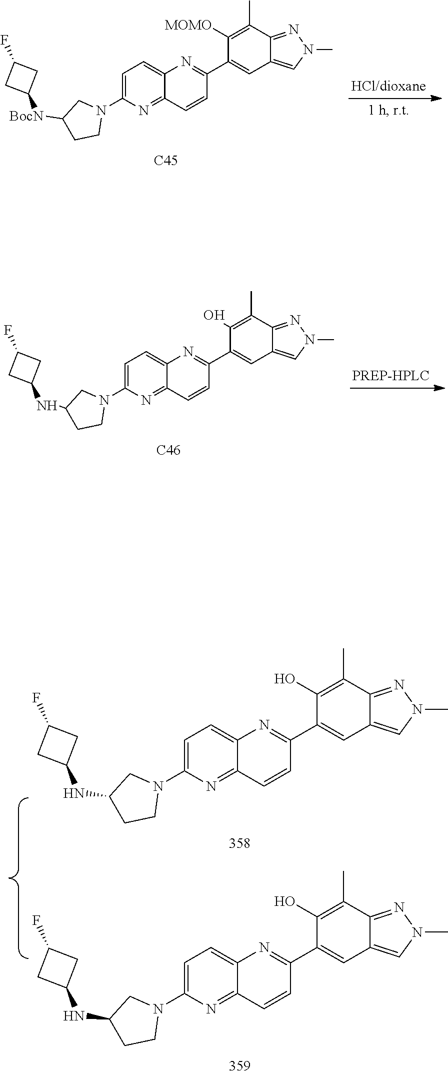

To a stirred solution of tert-butyl N-(1-{6-[6-(methoxymethoxy)-2,7-dimethylindazol-5-yl]-1,5-naphthyridin-2-yl}pyrrolidin-3-yl)-N-[(1r,3r)-3-fluorocyclobutyl]carbamate (80 mg, 0.13 mmol, 1 equiv) in methanol (1 mL) was added HCl (gas) in 1,4-dioxane (1 mL) in portions at room temperature under nitrogen atmosphere. The resulting mixture was stirred for 1 hr at room temperature. The resulting mixture was concentrated under reduced pressure to give a residue. The residue was purified by prep-HPLC (Condition 2, Gradient 14) followed by chiral prep-HPLC (Condition 4, Gradient 4) to afford 2,7-dimethyl-5-{6-[(3S)-3-{[(1r,3r)-3-fluorocyclobutyl]amino}pyrrolidin-1-yl]-1,5-naphthyridin-2-yl}indazol-6-ol (2.8 mg, 5%) and 2,7-dimethyl-5-{6-[(3R)-3-{[(1r,3r)-3-fluorocyclobutyl]amino}pyrrolidin-1-yl]-1,5-naphthyridin-2-yl}indazol-6-ol (2.1 mg, 3%) as solids. Compound 358: LCMS (ES, m/z): 447 [M+H]⁺. ¹H NMR (400 MHz, DMSO-d₆) δ 14.32 (s, 1H), 8.40-8.33 (t, J=4.7 Hz, 3H), 8.14-8.06 (d, J=9.2 Hz, 2H), 7.17 (d, J=9.3 Hz, 1H), 5.27 (s, 1H), 4.13 (s, 3H), 3.74-3.57 (s, 4H), 3.43 (s, 1H), 2.38 (s, 6H), 2.16 (s, 3H), 1.86 (s, 1H). Compound 359: LCMS (ES, m/z): 447 [M+H]⁺. ¹H NMR (400 MHz, DMSO-d₆) δ 14.32 (s, 1H), 8.40-8.33 (t, J=4.7 Hz, 3H), 8.14-8.06 (d, J=9.2 Hz, 2H), 7.17 (d, J=9.3 Hz, 1H), 5.27 (s, 1H), 4.13 (s, 3H), 3.74-3.57 (s, 4H), 3.43 (s, 1H), 2.38 (s, 6H), 2.16 (s, 3H), 1.86 (s, 1H).

### Example 123: Synthesis of Compounds 348 and 350

### Synthesis of Intermediate C47

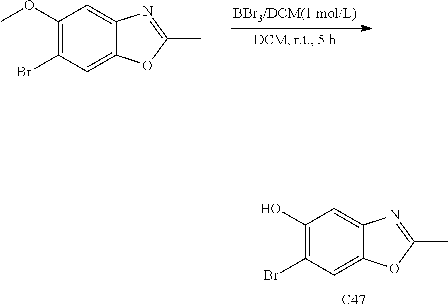

A mixture of 6-bromo-5-methoxy-2-methyl-1,3-benzoxazole (250 mg, 1.033 mmol, 1 equiv), DCM (5 mL), and BBr₃ (1293.62 mg, 5.165 mmol, 5 equiv, 1 mol/L in DCM) was stirred for 5 h at room temperature. The reaction mixture was quenched with methanol (20 mL) at 0° C., then concentrated under reduced pressure to give a residue. The residue was dissolved in CH₂Cl₂ (20 mL), basified to pH 7 with saturated NaHCO₃ (aq.), and extracted with CH₂Cl₂ (3×10 mL). The organic layers were combined, washed with brine (1×20 mL), dried over anhydrous Na₂SO₄, and filtered. After filtration, the filtrate was concentrated under reduced pressure to give 6-bromo-2-methyl-1,3-benzoxazol-5-ol (200 mg, 85%). LCMS (ES, m/z):228 [M+H]⁺.

### Synthesis of Intermediate C48

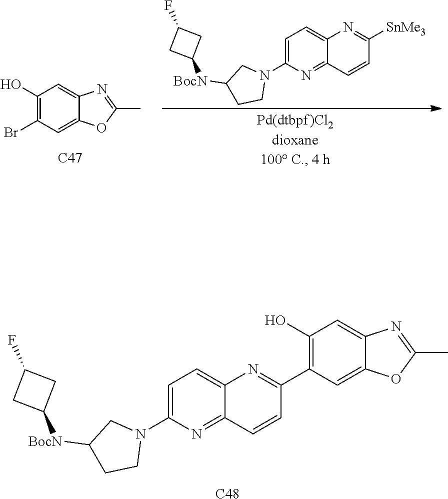

6-bromo-2-methyl-1,3-benzoxazol-5-ol (150 mg, 0.658 mmol, 1 equiv), tert-butyl N-[(1r,3r)-3-fluorocyclobutyl]-N-{1-[6-(trimethylstannyl)-1,5-naphthyridin-2-yl]pyrrolidin-3-yl}carbamate (361.30 mg, 0.658 mmol, 1 equiv), dioxane (15 mL, 177.059 mmol, 269.18 equiv), and Pd(dppf)Cl₂·CH₂Cl₂ (53.58 mg, 0.066 mmol, 0.1 equiv) were combined at room temperature. The resulting mixture was stirred for 4 hr at 100° C. under nitrogen atmosphere, then cooled to room temperature and quenched with water (30 mL) at room temperature and extracted with ethyl acetate (3×20 mL). The organic layers were combined, washed with brine (1×30 mL), dried over anhydrous Na₂SO₄, and filtered. After filtration, the filtrate was concentrated under reduced pressure to give a residue. The residue was purified by Prep-TLC (CH₂Cl₂/MeOH=10:1) to afford tert-butyl N-{1-[6-(5-hydroxy-2-methyl-1,3-benzoxazol-6-yl)-1,5-naphthyridin-2-yl]pyrrolidin-3-yl}-N-[(1r,3r)-3-fluorocyclobutyl]carbamate (70 mg, 16%) as a solid. LCMS (ES, m/z):534 [M+H]⁺.

### Synthesis of Compounds 348 and 350

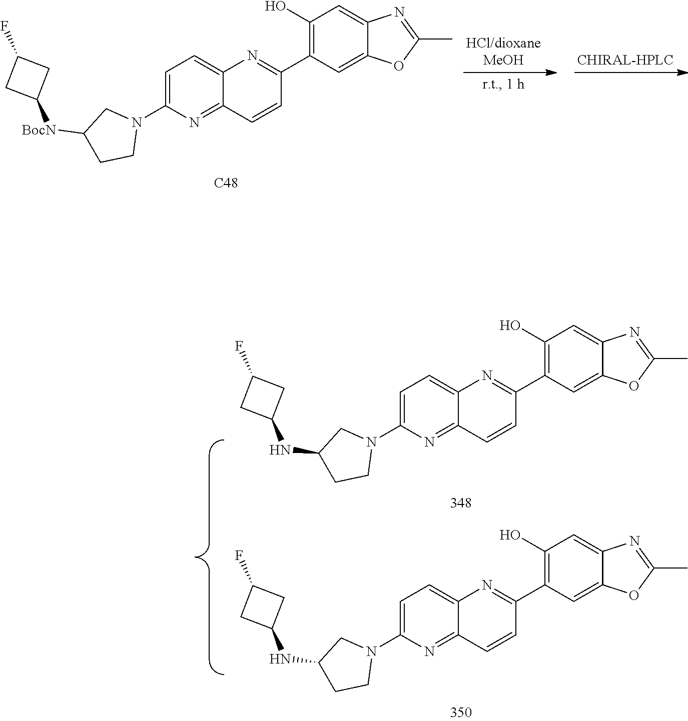 [SMILES: Cc1nc2cc(O)c(-c3ccc4nc(N5CCC(N(C(=O)OC(C)(C)C)[C@H]6C[C@H](F)C6)C5)ccc4n3)cc2o1 ; "C48" ; Cc1nc2cc(O)c(-c3ccc4nc(N5CC[C@@H](N[C@H]6C[C@H](F)C6)C5)ccc4n3)cc2o1 ; Cc1nc2cc(O)c(-c3ccc4nc(N5CC[C@H](N[C@H]6C[C@H](F)C6)C5)ccc4n3)cc2o1]

A mixture of tert-butyl N-{1-[6-(5-hydroxy-2-methyl-1,3-benzoxazol-6-yl)-1,5-naphthyridin-2-yl]pyrrolidin-3-yl}-N-[(1r,3r)-3-fluorocyclobutyl]carbamate (65 mg, 0.122 mmol, 1 equiv) and HCl (gas) in 1,4-dioxane (3 mL) was stirred for 1 h at room temperature. The resulting mixture was concentrated under reduced pressure to give a residue. The residue was purified by prep-HPLC(Condition 1, Gradient 28), followed by chiral-HPLC (Condition 4, Gradient 9) to afford 2-methyl-6-{6-[(3R)-3-{[(1r,3r)-3-fluorocyclobutyl]amino}pyrrolidin-1-yl]-1,5-naphthyridin-2-yl}-1,3-benzoxazol-5-ol (5.5 mg, 10%) and 2-methyl-6-{6-[(3S)-3-{[(1r,3r)-3-fluorocyclobutyl]amino}pyrrolidin-1-yl]-1,5-naphthyridin-2-yl}-1,3-benzoxazol-5-ol (6.5 mg, 12%) as solids. Compound 348: LCMS (ES, m/z):434 [M+H]⁺. ¹H NMR (400 MHz, DMSO-d₆) δ 14.51 (s, 1H), 8.46-8.39 (m, 2H), 8.16-8.05 (m, 2H), 7.17 (d, J=9.3 Hz, 1H), 7.12 (s, 1H), 5.19 (ddq, J=57.0, 6.4, 3.3 Hz, 1H), 3.79-3.63 (m, 2H), 3.57 (s, 2H), 3.40 (s, 2H), 2.61 (s, 3H), 2.40-2.28 (m, 2H), 2.25-2.05 (m, 3H), 1.85 (s, 1H). Compound 350: LCMS (ES, m/z):434 [M+H]⁺. ¹H NMR (400 MHz, DMSO-d₆) δ 14.52 (s, 1H), 8.46-8.39 (m, 2H), 8.10 (ddd, J=17.2, 9.2, 0.8 Hz, 2H), 7.17 (d, J=9.3 Hz, 1H), 7.12 (s, 1H), 5.19 (dtt, J=56.9, 6.4, 3.6 Hz, 1H), 3.76-3.69 (m, 2H), 3.56 (s, 2H), 3.38 (s, 2H), 2.61 (s, 3H), 2.43-2.27 (m, 2H), 2.23-2.04 (m, 3H), 1.84 (s, 1H).

### Example 124: Synthesis of Compounds 353 and 357

### Synthesis of Intermediate C49

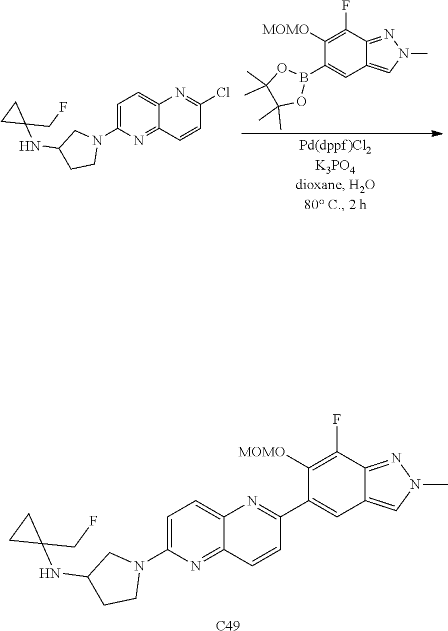

A solution of 1-(6-chloro-1,5-naphthyridin-2-yl)-N-[1-(fluoromethyl)cyclopropyl]pyrrolidin-3-amine (190 mg, 0.592 mmol, 1 equiv) in dioxane (4 mL) and water (0.8 mL) was treated with K₃PO₄ (377.15 mg, 1.776 mmol, 3 equiv) and Pd(dppf)Cl₂ (43.34 mg, 0.059 mmol, 0.1 equiv) at room temperature under nitrogen atmosphere. To the reaction mixture was added 7-fluoro-6-(methoxymethoxy)-2-methyl-5-(4,4,5,5-tetramethyl-1,3,2-dioxaborolan-2-yl) indazole (238.92 mg, 0.710 mmol, 1.2 equiv) in portions at room temperature. The resulting mixture was stirred for 2 h at 80° C., then concentrated under vacuum to give a residue. The residue was purified by silica gel column chromatography, eluted with CH₂Cl₂/MeOH (9:1) to afford 1-{6-[7-fluoro-6-(methoxymethoxy)-2-methylindazol-5-yl]-1,5-naphthyridin-2-yl}-N-[1-(fluoromethyl)cyclopropyl]pyrrolidin-3-amine (180 mg, 61%) as a solid.

### Synthesis of Compound 353 and 357

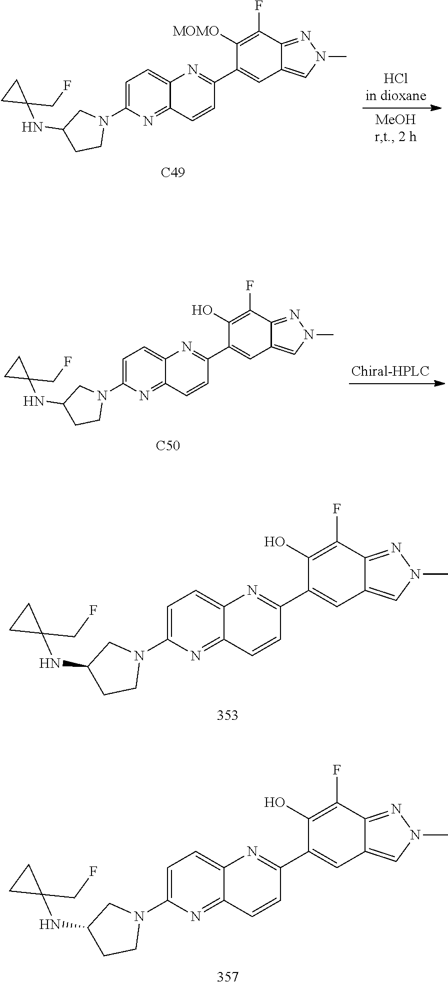

A mixture of 1-{6-[7-fluoro-6-(methoxymethoxy)-2-methylindazol-5-yl]-1,5-naphthyridin-2-yl}-N-[1-(fluoromethyl)cyclopropyl]pyrrolidin-3-amine (180 mg, 0.364 mmol, 1 equiv) and HCl (gas) in 1,4-dioxane (2 mL, 65.826 mmol, 180.85 equiv) in methanol (2 mL, 0.062 mmol, 0.17 equiv) was stirred for 2 hr at room temperature under nitrogen atmosphere. The resulting mixture was concentrated under vacuum to give a residue. The residue product was purified by reverse phase flash chromatography (Condition 5, Gradient 2), followed by chiral-HPLC (Condition 4, Gradient 1) to afford 7-fluoro-5-{6-[(3R)-3-{[1-(fluoromethyl)cyclopropyl]amino}pyrrolidin-1-yl]-1,5-naphthyridin-2-yl}-2-methylindazol-6-ol (8.6 mg, 5%) and 7-fluoro-5-{6-[(3S)-3-{[1-(fluoromethyl) cyclopropyl]amino}pyrrolidin-1-yl]-1,5-naphthyridin-2-yl}-2-methylindazol-6-ol (12.8 mg, 8%) as solids. Compound 353: LCMS (ES, m/z): 451 [M+H]⁺. ¹H NMR (400 MHz, DMSO-d₆) δ 14.43 (s, 1H), 8.48 (d, J=2.8 Hz, 1H), 8.41 (t, J=4.7 Hz, 2H), 8.11 (dd, J=20.3, 9.1 Hz, 2H), 7.16 (d, J=9.3 Hz, 1H), 4.47 (s, 1H), 4.35 (s, 1H), 4.16 (s, 3H), 3.82-3.75 (m, 1H), 3.70 (s, 2H), 3.55 (s, 1H), 2.67 (s, 1H), 2.15 (s, 1H), 1.90 (s, 1H), 0.62 (s, 4H). Compound 357: LCMS (ES, m/z): 451 [M+H]⁺. ¹H NMR (400 MHz, DMSO-d₆) δ 14.42 (s, 1H), 8.48 (d, J=2.7 Hz, 1H), 8.41 (t, J=4.7 Hz, 2H), 8.14 (d, J=9.2 Hz, 1H), 8.09 (d, J=9.0 Hz, 1H), 7.16 (d, J=9.2 Hz, 1H), 4.47 (s, 1H), 4.35 (s, 1H), 4.16 (s, 3H), 3.79 (s, 1H), 3.70 (s, 2H), 3.54 (s, 1H), 3.08 (s, 1H), 2.79-2.58 (m, 1H), 2.14 (s, 1H), 1.91 (s, 1H), 0.62 (s, 4H).

### Example 125: Synthesis of Compounds 354 and 355

### Synthesis of Intermediate C51

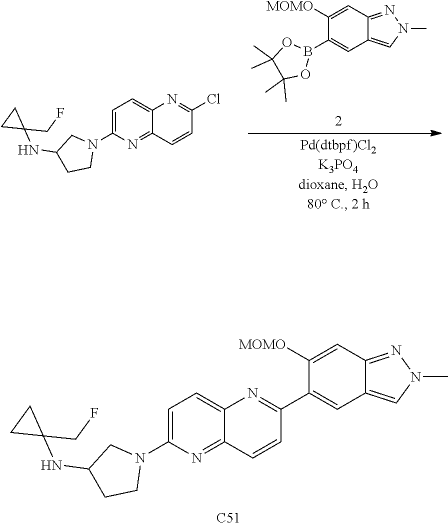

A mixture of 1-(6-chloro-1,5-naphthyridin-2-yl)-N-[1-(fluoromethyl)cyclopropyl]pyrrolidin-3-amine (170 mg, 0.530 mmol, 1 equiv) and Pd(dtbpf)Cl₂ (34.54 mg, 0.053 mmol, 0.1 equiv) in dioxane (2 mL) and water (2 mL) was treated with K₃PO₄ (337.45 mg, 1.590 mmol, 3 equiv) at room temperature under nitrogen atmosphere. To the reaction mixture was added 6-(methoxymethoxy)-2-methyl-5-(4,4,5,5-tetramethyl-1,3,2-dioxaborolan-2-yl) indazole (202.33 mg, 0.636 mmol, 1.2 equiv) in portions at room temperature. The resulting mixture was stirred for 2 h at 80° C., then concentrated under vacuum to give a residue. The residue was purified by silica gel column chromatography, eluted with CH₂Cl₂/MeOH (9:1) to afford N-[1-(fluoromethyl)cyclopropyl]-1-{6-[6-(methoxymethoxy)-2-methylindazol-5-yl]-1,5-naphthyridin-2-yl}pyrrolidin-3-amine (160 mg, 63%) as a solid.

### Synthesis of Compounds 354 and 355

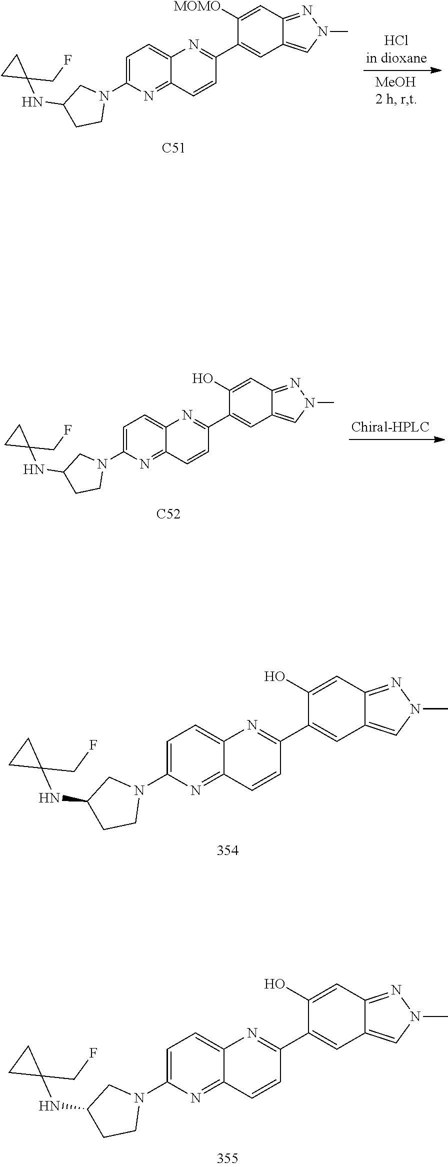

A mixture of N-[1-(fluoromethyl)cyclopropyl]-1-{6-[6-(methoxymethoxy)-2-methylindazol-5-yl]-1,5-naphthyridin-2-yl}pyrrolidin-3-amine (160 mg, 0.336 mmol, 1 equiv) and HCl (gas) in 1,4-dioxane (2 mL) in methanol (2 mL, 0.062 mmol, 0.19 equiv) was stirred for 2 h at room temperature under nitrogen atmosphere. The resulting mixture was concentrated under vacuum to give a residue. The residue was purified by reverse phase flash chromatography (Condition 5, Gradient 3) followed by chiral-HPLC (Condition 4, Gradient 1) to afford 5-{6-[(3R)-3-{[1-(fluoromethyl)cyclopropyl]amino}pyrrolidin-1-yl]-1,5-naphthyridin-2-yl}-2-methylindazol-6-ol (10.4 mg, 7%) and 5-{6-[(3S)-3-{[1-(fluoromethyl)cyclopropyl]amino}pyrrolidin-1-yl]-1,5-naphthyridin-2-yl}-2-methylindazol-6-ol (8.6 mg, 6%) as solids. Compound 354: LCMS (ES, m/z): 433 [M+H]⁺. ¹H NMR (400 MHz, DMSO-d₆) δ 13.97 (s, 1H), 8.54 (s, 1H), 8.39 (d, J=9.2 Hz, 1H), 8.35 (s, 1H), 8.08 (dd, J=13.7, 9.2 Hz, 2H), 7.13 (d, J=9.3 Hz, 1H), 6.87 (s, 1H), 4.47 (s, 1H), 4.35 (s, 1H), 4.11 (s, 3H), 3.77 (d, J=9.9 Hz, 1H), 3.70 (s, 2H), 3.54 (d, J=9.1 Hz, 1H), 3.32 (s, 1H), 2.71 (s, 1H), 2.19-2.11 (m, 1H), 1.91 (s, 1H), 0.62 (s, 4H). Compound 355: LCMS (ES, m/z): 433 [M+H]⁺. ¹H NMR (400 MHz, DMSO-d₆) δ 13.95 (s, 1H), 8.54 (s, 1H), 8.40 (d, J=9.2 Hz, 1H), 8.35 (s, 1H), 8.19-8.03 (m, 2H), 7.16 (s, 1H), 6.87 (s, 1H), 4.42 (d, J=49.9 Hz, 2H), 4.11 (s, 3H), 3.71 (s, 3H), 3.56 (s, 1H), 2.92 (dd, J=12.8, 6.8 Hz, 1H), 2.16 (s, 1H), 1.91 (s, 1H), 0.63 (s, 4H).

### Example 126: Synthesis of Compound 382

### Synthesis of Intermediate C53

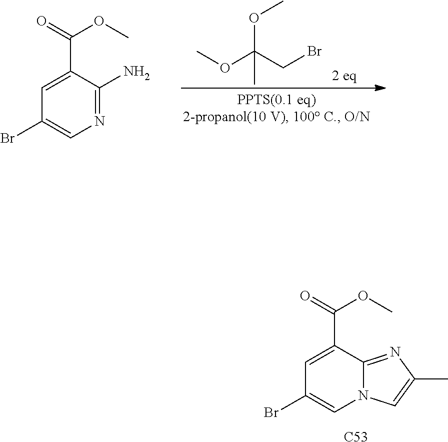

A mixture of methyl 2-amino-5-bromopyridine-3-carboxylate (25.0 g, 108.20 mmol, 1 equiv) and PPTS (2.72 g, 10.82 mmol, 0.1 equiv) in 2-Propanol (250 mL) was stirred overnight at 100° C., then concentrated under vacuum to give a residue. The residue was purified by silica gel column chromatography, eluted with CH₂Cl₂/MeOH (5:1) to afford methyl 6-bromo-2-methylimidazo[1,2-a]pyridine-8-carboxylate (23.4 g, 80%) as a solid. LCMS (ES, m/z): 269 [M+H]⁺.

### Synthesis of Intermediate C55

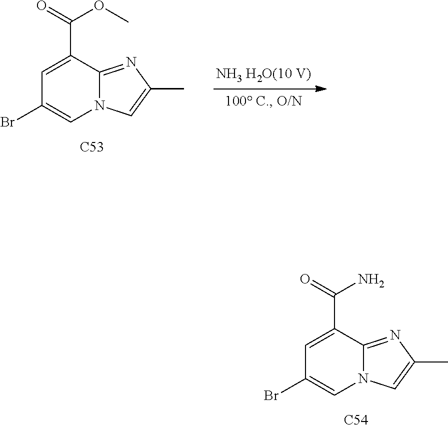

A solution of methyl 6-bromo-2-methylimidazo[1,2-a]pyridine-8-carboxylate (23.4 g, 86.95 mmol, 1 equiv) in NH₃·H₂O (234 mL) was stirred overnight at 100° C. in a pressure tank. A precipitate formed that was collected by filtration and washed with water (3×200 mL) to afford 6-bromo-2-methylimidazo[1,2-a]pyridine-8-carboxamide (11.4 g, 52%) as a solid. LCMS (ES, m/z): 254 [M+H]⁺.

### Synthesis of Intermediate C55

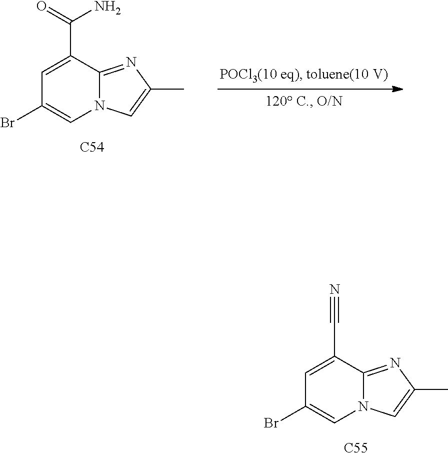

A mixture of 6-bromo-2-methylimidazo[1,2-a]pyridine-8-carboxamide (11.4 g, 44.86 mmol, 1 equiv) and POCl₃ (68.8 g, 448.67 mmol, 10 equiv) in toluene (114 mL) was stirred overnight at 120° C., then concentrated under vacuum. The resulting mixture was diluted with water (50 mL) and basified to pH 8 with saturated NaHCO₃ (aq.). A precipitate formed that was collected by filtration and washed with water (3×50 mL) to afford 6-bromo-2-methylimidazo[1,2-a]pyridine-8-carbonitrile (8.63 g, 82%) as a solid. LCMS (ES, m/z): 236 [M+H]⁺.

### Synthesis of Intermediate C56

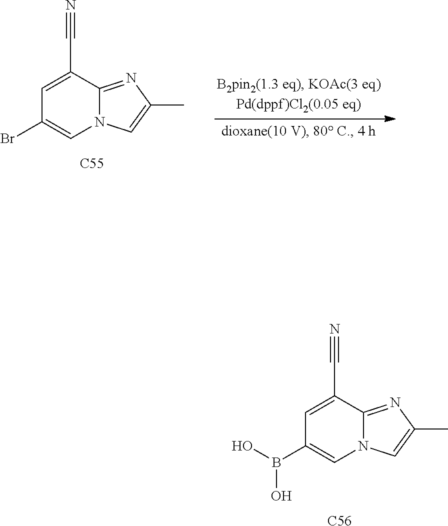

A mixture of 6-bromo-2-methylimidazo[1,2-a]pyridine-8-carbonitrile (3.13 g, 13.25 mmol, 1 equiv), bis(pinacolato)diboron (4.38 g, 17.23 mmol, 1.3 equiv), potassium acetate (3.90 g, 39.77 mmol, 3 equiv), and Pd(dppf)Cl₂·CH₂Cl₂ (0.54 g, 0.66 mmol, 0.05 equiv) in dioxane (40 mL) was stirred for 4 hr at 80° C. under nitrogen atmosphere. LCMS (ES, m/z): 202 [M+H]⁺.

### Synthesis of Intermediate C53

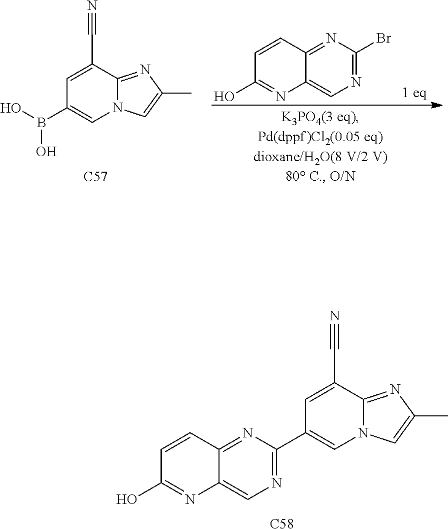

A mixture of 2-bromopyrido[3,2-d]pyrimidin-6-ol (3.0 g, 13.27 mmol, 1 equiv), 8-cyano-2-methylimidazo[1,2-a]pyridin-6-ylboronic acid (2.93 g, 14.59 mmol, 1.1 equiv), Pd(dppf)Cl₂·CH₂Cl₂ (0.54 g, 0.664 mmol, 0.05 equiv), and K₃PO₄ (8.45 g, 39.81 mmol, 3 equiv) in dioxane (48 mL) and water (12 mL) was stirred overnight at 80° C. under nitrogen atmosphere. The resulting mixture was diluted with water (100 mL). A precipitate formed that was collected by filtration and washed with water (3×50 mL) to afford 6-{6-hydroxypyrido[3,2-d]pyrimidin-2-yl}-2-methylimidazo[1,2-a]pyridine-8-carbonitrile (2.15 g, 54%) as a solid. LCMS (ES, m/z): 303 [M+H]⁺.

### Synthesis of Intermediate C59

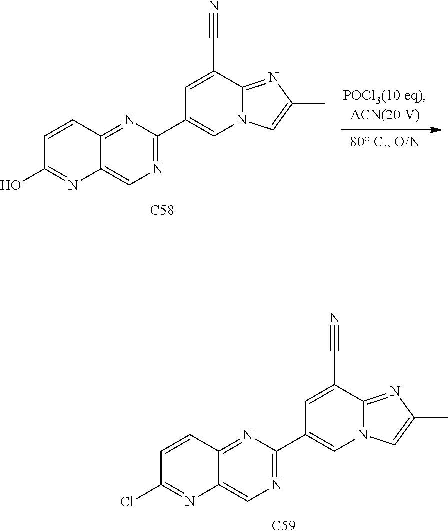 [SMILES: Cc1cn2cc(-c3ncc4nc(O)ccc4n3)cc(C#N)c2n1 ; "C58" ; Cc1cn2cc(-c3ncc4nc(Cl)ccc4n3)cc(C#N)c2n1 ; "C59"]

A mixture of 6-{6-hydroxypyrido[3,2-d]pyrimidin-2-yl}-2-methylimidazo[1,2-a]pyridine-8-carbonitrile (900 mg, 2.97 mmol, 1 equiv) and POCl₃ (4564.6 mg, 29.77 mmol, 10 equiv) in acetonitrile (10 mL) was stirred overnight at 80° C. The resulting mixture was concentrated under vacuum to give a residue. The residue was basified to pH 8 with saturated NaHCO₃ (aq.). A precipitate formed that was collected by filtration and washed with water (3×30 mL) to afford 6-{6-chloropyrido[3,2-d]pyrimidin-2-yl}-2-methylimidazo[1,2-a]pyridine-8-carbonitrile (720 mg, 75%) as a solid. LCMS (ES, m/z): 321 [M+H]⁺.

### Synthesis of Intermediate C60

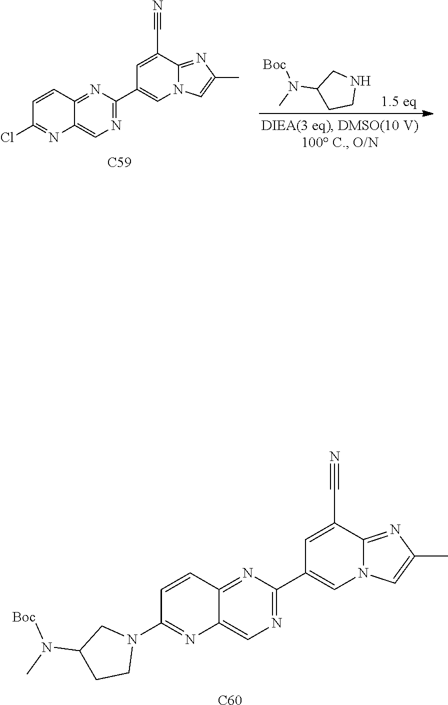

A mixture of 6-{6-chloropyrido[3,2-d]pyrimidin-2-yl}-2-methylimidazo[1,2-a]pyridine-8-carbonitrile (220 mg, 0.68 mmol, 1 equiv), tert-butyl N-methyl-N-(pyrrolidin-3-yl)carbamate (206.0 mg, 1.02 mmol, 1.5 equiv), and DIEA (265.9 mg, 2.05 mmol, 3 equiv) in DMSO (4 mL) was stirred overnight at 100° C. The resulting mixture was diluted with water (30 mL) and extracted with ethyl acetate (3×30 mL). The organic layers were combined, washed with brine (1×30 mL), dried over anhydrous Na₂SO₄, and filtered. After filtration, the filtrate was concentrated under reduced pressure to give a residue. The residue was purified by silica gel column chromatography, eluted with PE/EA (1:1) to afford tert-butyl N-[1-(2-{8-cyano-2-methylimidazo[1,2-a]pyridin-6-yl}pyrido[3,2-d]pyrimidin-6-yl)pyrrolidin-3-yl]-N-methylcarbamate (85.0 mg, 26%) as a solid. LCMS (ES, m/z): 485 [M+H]⁺.

### Synthesis of Compound 382

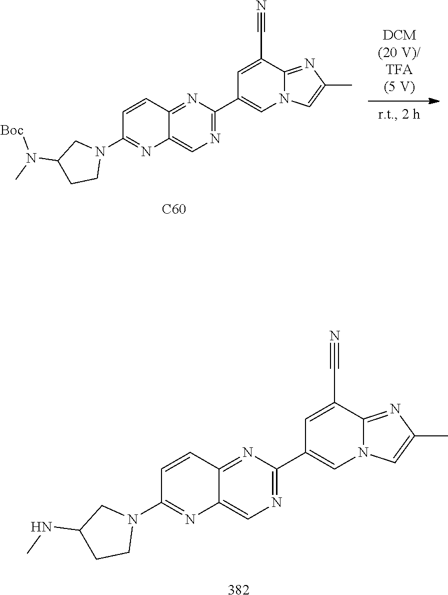

A mixture of tert-butyl N-[1-(2-{8-cyano-2-methylimidazo[1,2-a]pyridin-6-yl}pyrido[3,2-d]pyrimidin-6-yl)pyrrolidin-3-yl]-N-methylcarbamate (85.0 mg, 0.17 mmol, 1 equiv) in DCM (2 mL) and TFA (0.5 mL) was stirred for 2 hr at room temperature. The resulting mixture was concentrated under vacuum to give a residue. The residue was purified by prep-HPLC (Condition 1, Gradient 18) to afford 2-methyl-6-{6-[3-(methylamino)pyrrolidin-1-yl]pyrido[3,2-d]pyrimidin-2-yl}imidazo[1,2-a]pyridine-8-carbonitrile (29.0 mg, 43%) as a solid. LCMS (ES, m/z): 385 [M+H]⁺. ¹H NMR (400 MHz, DMSO-d₆) δ 9.65 (d, J=1.6 Hz, 1H), 9.10 (s, 1H), 8.54 (d, J=1.6 Hz, 1H), 8.06-7.96 (m, 2H), 7.31 (d, J=9.4 Hz, 1H), 3.77-3.49 (m, 3H), 3.45-3.40 (m, 2H), 2.43-2.39 (m, 3H), 2.37 (s, 3H), 2.19-2.11 (m, 1H), 1.92 (s, 1H).

### Example 127: Synthesis of Compound 335

### Synthesis of Intermediate C61

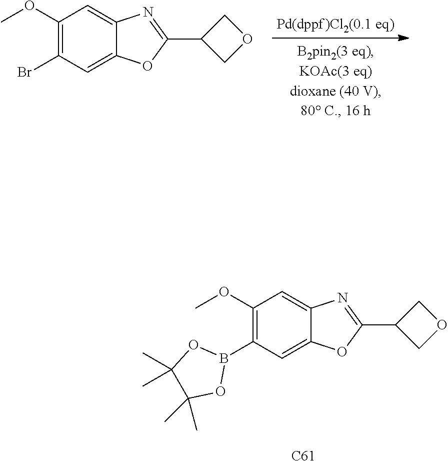 [SMILES: COc1cc2nc(C3COC3)oc2cc1Br ; "Pd(dppf)Cl2(0.1 eq)" ; COc1cc2nc(C3COC3)oc2cc1B1OC(C)(C)C(C)(C)O1 ; "C61"]

A mixture of 6-bromo-5-methoxy-2-(oxetan-3-yl)-1,3-benzoxazole (200 mg, 0.704 mmol, 1 equiv), potassium acetate (207 mg, 2.112 mmol, 3 equiv), 4,4,5,5-tetramethyl-2-(tetramethyl-1,3,2-dioxaborolan-2-yl)-1,3,2-dioxaborolane (536 mg, 2.112 mmol, 3 equiv), and Pd(DtBPF)Cl₂ (46 mg, 0.070 mmol, 0.1 equiv) in 1,4-dioxane (8 mL) was stirred for 16 hr at 80° C. under nitrogen atmosphere, then cooled to room temperature. The resulting mixture was poured into the water (40 mL) and extracted with ethyl acetate (1×40 mL). The organic layer was dried over anhydrous Na₂SO₄, and filtered. After filtration, the filtrate was concentrated under reduced pressure to afford 5-methoxy-2-(oxetan-3-yl)-6-(4,4,5,5-tetramethyl-1,3,2-dioxaborolan-2-yl)-1,3-benzoxazole (588 mg) as a solid. LCMS (ES, m/z): 332 [M+H]⁺.

### Synthesis of Compound 335

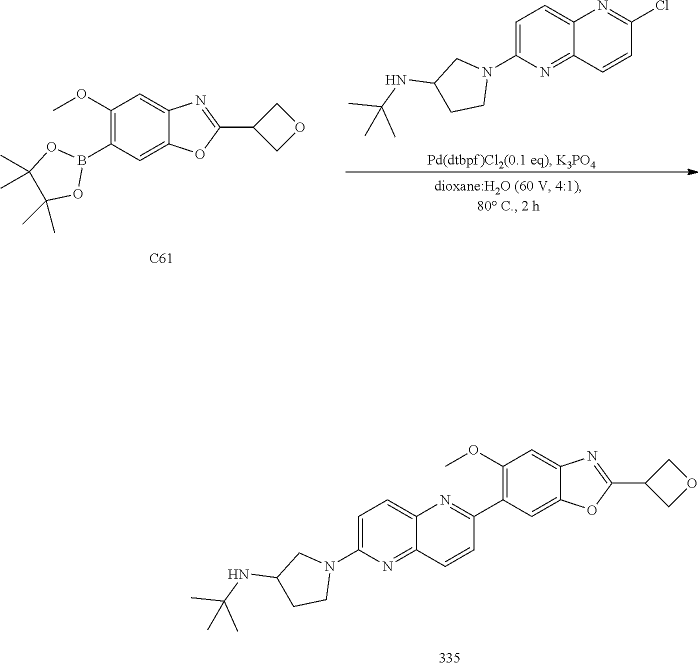 [SMILES: COc1cc2nc(C3COC3)oc2cc1B1OC(C)(C)C(C)(C)O1 ; CC(C)(C)NC1CCN(c2ccc3nc(Cl)ccc3n2)C1 ; "Pd(dtbpf)Cl2(0.1 eq), K3PO4" ; "C61" ; COc1cc2nc(C3COC3)oc2cc1-c1ccc2nc(N3CCC(NC(C)(C)C)C3)ccc2n1 ; "335"]

A mixture of N-tert-butyl-1-(6-chloro-1,5-naphthyridin-2-yl)pyrrolidin-3-amine (215 mg, 0.705 mmol, 1 equiv), 5-methoxy-2-(oxetan-3-yl)-6-(4,4,5,5-tetramethyl-1,3,2-dioxaborolan-2-yl)-1,3-benzoxazole (584 mg, 1.762 mmol, 2.5 equiv), Pd(DtBPF)Cl₂ (46 mg, 0.070 mmol, 0.1 equiv), and K₃PO₄ (449 mg, 2.115 mmol, 3 equiv) in 1,4-dioxane (10.3 mL) and water (2.6 mL) was stirred for 2 h at 80° C. under nitrogen atmosphere. The reaction mixture was cooled to room temperature, then extracted with ethyl acetate (1×50 mL). The organic layer was dried over anhydrous Na₂SO₄ and filtered. After filtration, the filtrate was concentrated under reduced pressure to give a residue. The residue was purified by silica gel column chromatography, eluted with PE/EA (1:1), followed by prep-HPLC (Condition 1, Gradient 21) to afford N-tert-butyl-1-{6-[5-methoxy-2-(oxetan-3-yl)-1,3-benzoxazol-6-yl]-1,5-naphthyridin-2-yl}pyrrolidin-3-amine (8.9 mg, 3%) as a solid. LCMS (ES, m/z):474 [M+H]⁺. ¹H NMR (400 MHz, DMSO-d₆) δ 8.10-7.91 (m, 3H), 7.91 (dd, J=8.8, 0.8 Hz, 1H), 7.52 (s, 1H), 7.10 (d, J=9.4 Hz, 1H), 4.99 (dd, J=8.6, 5.9 Hz, 2H), 4.90 (dd, J=6.6, 5.9 Hz, 2H), 4.63 (tt, J=8.5, 6.6 Hz, 1H), 3.90 (s, 3H), 3.85 (s, 1H), 3.71 (s, 1H), 3.51 (dt, J=17.3, 8.4 Hz, 2H), 3.17-3.08 (m, 1H), 2.18 (d, J=8.9 Hz, 1H), 1.81-1.71 (m, 2H), 1.09 (s, 9H).

### Example 128: Synthesis of Compound 341

### Synthesis of Intermediate C62

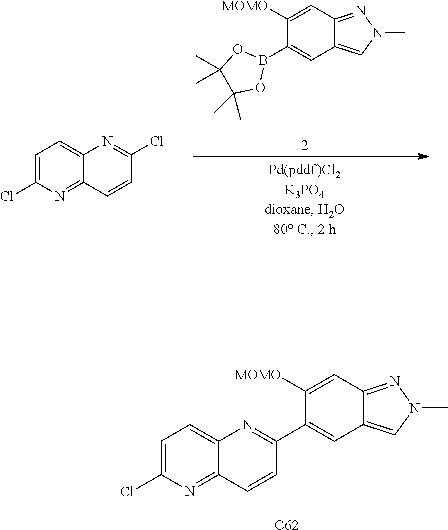

A mixture of 2,6-dichloro-1,5-naphthyridine (1 g, 5.024 mmol, 1 equiv) and 6-(methoxymethoxy)-2-methyl-5-(4,4,5,5-tetramethyl-1,3,2-dioxaborolan-2-yl) indazole (1.92 g, 6.029 mmol, 1.2 equiv) in dioxane (10 mL) and water (2 mL) was treated with K₃PO₄ (3.20 g, 15.072 mmol, 3 equiv) and Pd(dppf)Cl₂ (0.41 g, 0.502 mmol, 0.1 equiv) at room temperature under nitrogen atmosphere. The reaction was stirred for 2 hr at 80° C., then concentrated under vacuum to give a residue. The residue was purified by silica gel column chromatography, eluted with PE/EA (1:1) to afford 2-chloro-6-[6-(methoxymethoxy)-2-methylindazol-5-yl]-1,5-naphthyridine (550 mg, 31%) as a solid.

### Synthesis of Intermediate C63

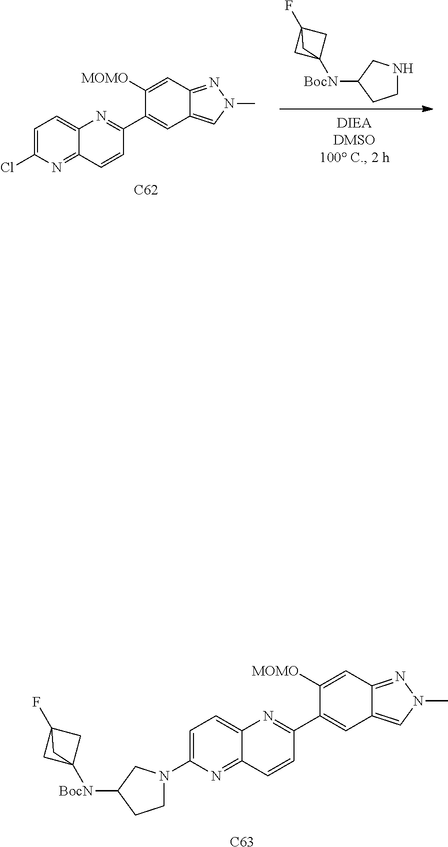

To a solution of 2-chloro-6-[6-(methoxymethoxy)-2-methylindazol-5-yl]-1,5-naphthyridine (550 mg, 1.550 mmol, 1 equiv) in DMSO (10 mL) was added DIEA (601.08 mg, 4.650 mmol, 3 equiv), followed by tert-butyl N-{3-fluorobicyclo[1.1.1]pentan-1-yl}-N-(pyrrolidin-3-yl)carbamate (502.92 mg, 1.860 mmol, 1.2 equiv) in portions at room temperature. The reaction mixture was stirred for 2 hr at 100° C. The resulting mixture was extracted with ethyl acetate (2×30 mL). The organic layers were combined, washed with brine (2×30 mL), dried over anhydrous Na₂SO₄, and filtered. After filtration, the filtrate was concentrated under reduced pressure to give a residue. The residue was purified by silica gel column chromatography, eluted with CH₂Cl₂/PE (1:1) to afford tert-butyl N-{3-fluorobicyclo[1.1.1]pentan-1-yl}-N-(1-{6-[6-(methoxymethoxy)-2-methylindazol-5-yl]-1,5-naphthyridin-2-yl}pyrrolidin-3-yl)carbamate (60 mg, 7%) as a solid.

### Synthesis of Compound 341

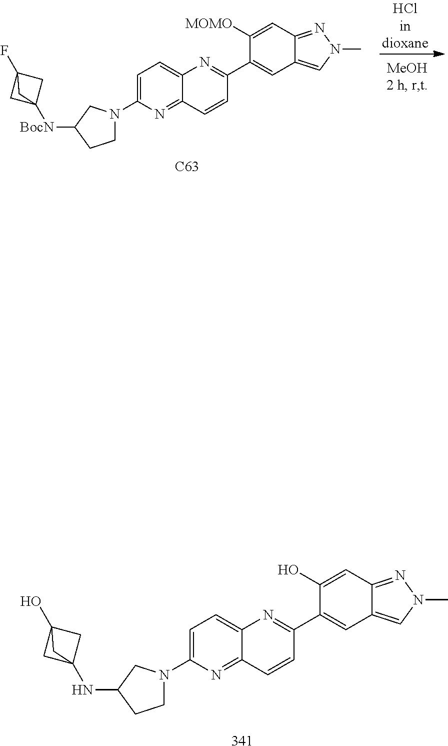

A mixture of tert-butyl N-{3-fluorobicyclo[1.1.1]pentan-1-yl}-N-(1-{6-[6-(methoxymethoxy)-2-methylindazol-5-yl]-1,5-naphthyridin-2-yl}pyrrolidin-3-yl)carbamate (60 mg, 0.102 mmol, 1 equiv) and HCl (gas) in 1,4-dioxane (1 mL) in methanol (1 mL) was stirred for 2 h at room temperature under nitrogen atmosphere. The resulting mixture was concentrated under vacuum to give a residue. The residue was purified by reverse phase flash chromatography (Condition 5, Gradient 4) to afford 5-{6-[3-({3-hydroxybicyclo[1.1.1]pentan-1-yl}amino)pyrrolidin-1-yl]-1,5-naphthyridin-2-yl}-2-methylindazol-6-ol (9.1 mg, 20%) as a solid. LCMS (ES, m/z): 443 [M+H]+. ¹H NMR (400 MHz, DMSO-d₆) δ 13.90 (s, 1H), 11.02 (d, J=8.5 Hz, 1H), 8.55 (s, 1H), 8.42 (d, J=9.3 Hz, 1H), 8.35 (s, 1H), 8.19-8.12 (m, 1H), 8.15-8.06 (m, 1H), 7.22 (d, J=9.3 Hz, 1H), 6.90-6.84 (m, 1H), 5.01 (s, 1H), 4.45 (q, J=5.9 Hz, 1H), 4.11 (s, 3H), 3.94 (dd, J=10.9, 6.1 Hz, 1H), 3.71 (q, J=6.9 Hz, 2H), 3.48 (dd, J=11.0, 4.7 Hz, 1H), 2.40-2.32 (m, 1H), 2.05 (s, 3H), 1.99-1.90 (m, 1H), 1.85 (s, 3H).

### Example 129: Synthesis of Compound 360

### Synthesis of Intermediate C64

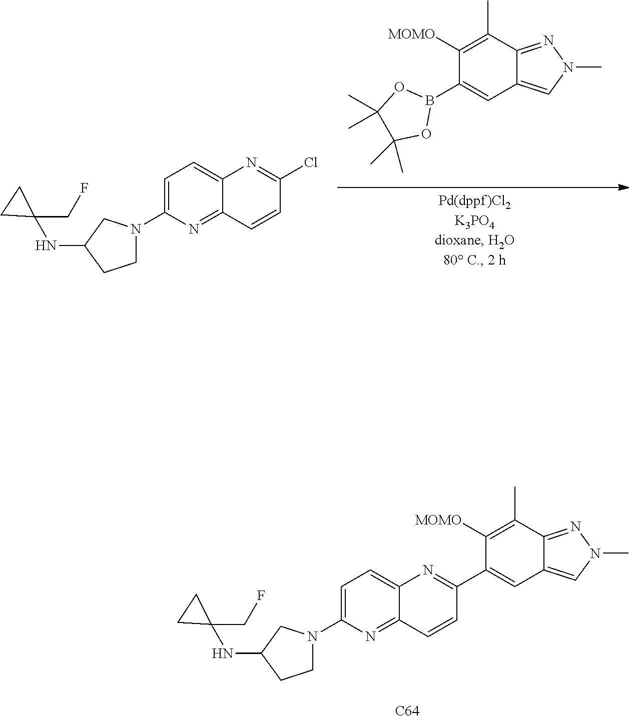

To a mixture of 1-(6-chloro-1,5-naphthyridin-2-yl)-N-[1-(fluoromethyl)cyclopropyl]pyrrolidin-3-amine (190 mg, 0.592 mmol, 1 equiv) and K₃PO₄ (377.15 mg, 1.776 mmol, 3 equiv) in dioxane (4 mL) and water (0.8 mL) was added Pd(dppf)Cl₂ (43.34 mg, 0.059 mmol, 0.1 equiv), followed by 6-(methoxymethoxy)-2,7-dimethyl-5-(4,4,5,5-tetramethyl-1,3,2-dioxaborolan-2-yl) indazole (236.11 mg, 0.710 mmol, 1.2 equiv) at room temperature. The reaction mixture was stirred for 2 h at 80° C., then cooled to room temperature. The resulting mixture was concentrated under vacuum to give a residue. The residue was purified by silica gel column chromatography, eluted with CH₂Cl₂/MeOH (9:1) to afford N-[1-(fluoromethyl)cyclopropyl]-1-{6-[6-(methoxymethoxy)-2,7-dimethylindazol-5-yl]-1,5-naphthyridin-2-yl}pyrrolidin-3-amine (50 mg, 17%) as a solid.

### Synthesis of Compound 360

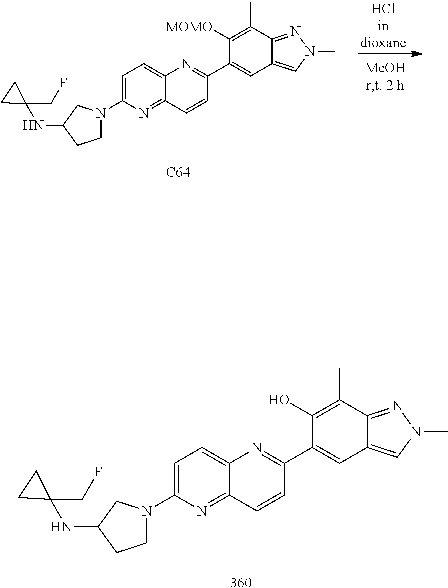

A mixture of N-[1-(fluoromethyl)cyclopropyl]-1-{6-[6-(methoxymethoxy)-2,7-dimethylindazol-5-yl]-1,5-naphthyridin-2-yl}pyrrolidin-3-amine (50 mg, 0.102 mmol, 1 equiv) and HCl (gas) in 1,4-dioxane (0.5 mL) in methanol (0.5 mL) was stirred for 2 hr at room temperature under nitrogen atmosphere. The resulting mixture was concentrated under vacuum to give a residue. The residue was purified by reverse phase flash chromatography (Condition 6, Gradient 1) to afford 5-[6-(3-{[1-(fluoromethyl)cyclopropyl]amino}pyrrolidin-1-yl)-1,5-naphthyridin-2-yl]-2,7-dimethylindazol-6-ol (3.3 mg, 7%) as a solid. LCMS (ES, m/z): 447 [M+H]⁺. ¹H NMR (400 MHz, DMSO-d₆) δ 14.32 (s, 1H), 8.42-8.36 (m, 2H), 8.32 (s, 1H), 8.09 (ddd, J=23.3, 9.2, 0.8 Hz, 2H), 7.14 (d, J=9.3 Hz, 1H), 4.47 (s, 1H), 4.35 (s, 1H), 4.13 (s, 3H), 3.77 (d, J=9.4 Hz, 1H), 3.70 (t, J=6.0 Hz, 2H), 3.53 (d, J=8.7 Hz, 1H), 3.34 (d, J=1.6 Hz, 1H), 2.71 (s, 1H), 2.38 (s, 3H), 2.15 (dd, J=12.2, 6.3 Hz, 1H), 1.92-1.86 (m, 1H), 0.67-0.59 (m, 4H).

### Example 130: Synthesis of Compound 337

### Synthesis of Intermediate C65

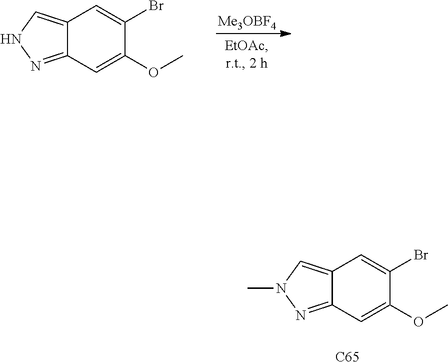

A mixture of 5-bromo-6-methoxy-2H-indazole (60 g, 264.246 mmol, 1 equiv) and tetrafluoroboranuide; trimethyloxidanium (117 g, 792.738 mmol, 3 equiv) in ethyl acetate (1.2 L) was stirred overnight at room temperature, then quenched with water (2 L) at room temperature. The resulting mixture was basified to pH 8 with saturated NaHCO₃ (aq.) and extracted with ethyl acetate (3×500 mL). The organic layers were combined, washed with brine (1×500 mL), dried over anhydrous Na₂SO₄, and filtered. After filtration, the filtrate was concentrated under reduced pressure to give a residue. The residue was purified by silica gel column chromatography, eluted with PE/EA (1:1) to afford 5-bromo-6-methoxy-2-methylindazole (45 g, 71%) as a solid. LCMS (ES, m/z): 241 [M+H]⁺.

### Synthesis of Intermediate C66

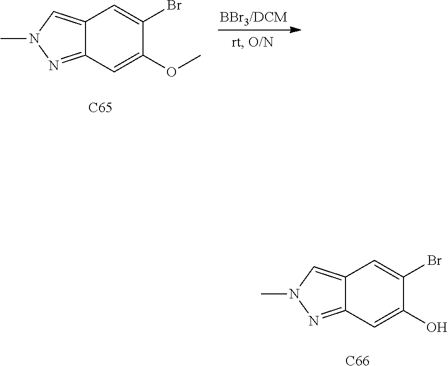

To a stirred solution of 5-bromo-6-methoxy-2-methylindazole (41 g, 170.062 mmol, 1 equiv) in DCM (800 mL) was added BBr₃ (85 g, 340.124 mmol, 2 equiv) dropwise at 0° C. The resulting mixture was stirred overnight at room temperature, then quenched with water/ice (2 L) at 5° C. and basified to pH 8 with saturated NaHCO₃ (aq.). A precipitate formed that was collected by filtration and washed with water (3×200 mL) to afford 5-bromo-2-methylindazol-6-ol (38.2 g, 99%) as a solid. LCMS (ES, m/z): 227 [M+H]⁺.

### Synthesis of Intermediate C67

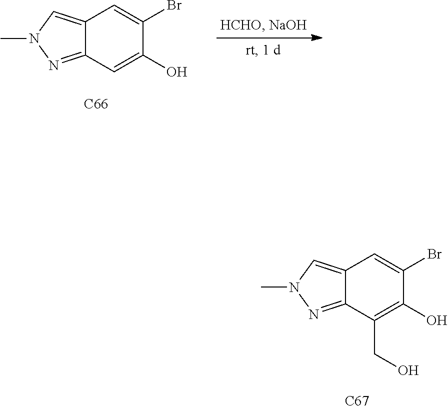

To a stirred solution of 5-bromo-2-methylindazol-6-ol (1.5 g, 6.606 mmol, 1 equiv) in water (8.1 mL) was added NaOH (2.5M) (0.26 g, 166.035 mmol, 1 equiv) dropwise at 5° C., then stirred for 0.5 h at 5° C. To the resulting mixture was added HCHO (0.54 g, 17.984 mmol, 2.72 equiv) dropwise at 5° C. and stirred for an additional 1.5 hr at room temperature. A precipitate formed that was collected by filtration and washed with water (2×5 mL) to afford 5-bromo-7-(hydroxymethyl)-2-methylindazol-6-ol (1.55 g, 91%) as a solid. LCMS (ES, m/z): 257 [M+H]⁺.

### Synthesis of Intermediate C68

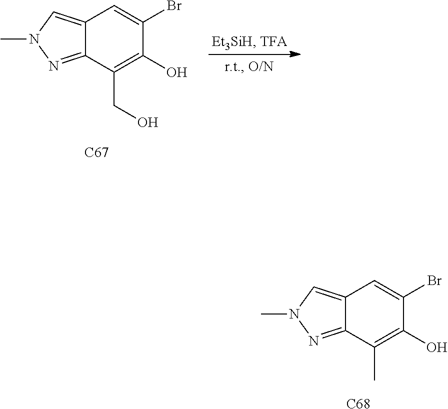

A mixture of 5-bromo-7-(hydroxymethyl)-2-methylindazol-6-ol (37.7 g, 146.643 mmol, 1 equiv) in Et₃S¹H (400 mL) and TFA (800 mL) was stirred overnight at room temperature. The resulting mixture was concentrated under reduced pressure to give a residue. The residue was basified to pH 8 with saturated NaHCO₃ (aq.) and extracted with ethyl acetate (3×200 mL). The organic layers were combined, washed with brine (1×300 mL), dried over anhydrous Na₂SO₄, and filtered. After filtration, the filtrate was concentrated under reduced pressure. A precipitate formed that was collected by filtration and washed with ethyl ether (3×50 mL) to afford 5-bromo-2,7-dimethylindazol-6-ol (28 g, 79%) as a solid. LCMS (ES, m/z): 241 [M+H]⁺.

### Synthesis of Intermediate C69

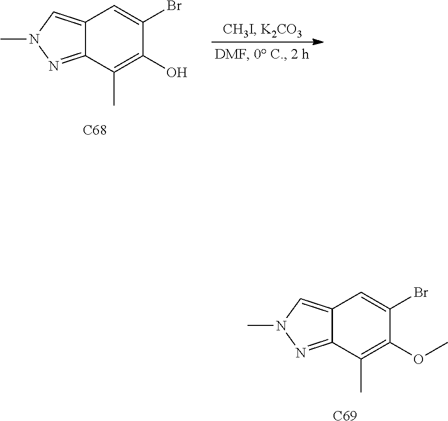

To a stirred mixture of 5-bromo-2,7-dimethylindazol-6-ol (18 g, 74.662 mmol, 1 equiv) and K₂CO₃ (12.4 g, 89.594 mmol, 1.2 equiv) in DMF (300 mL) was added CH₃I (53 g, 373.310 mmol, 5 equiv) dropwise at room temperature. The resulting mixture was stirred for 2 h at room temperature, then quenched with sat. NH₄Cl (aq.) (200 mL) at room temperature and extracted with ethyl acetate (3×100 mL). The organic layers were combined, washed with brine (1×200 mL), dried over anhydrous Na₂SO₄, and filtered. After filtration, the filtrate was concentrated under reduced pressure to give a residue. The residue was purified by silica gel column chromatography, eluted with PE/EA (4:1) to afford 5-bromo-6-methoxy-2,7-dimethylindazole (16 g, 84%) as a solid. LCMS (ES, m/z): 254 [M+H]⁺.

### Synthesis of Intermediate C70

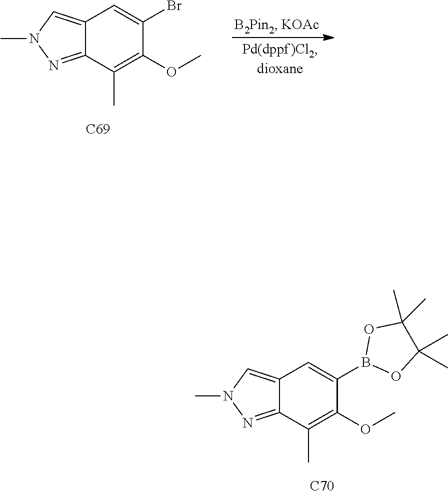

To a stirred mixture of 5-bromo-6-methoxy-2,7-dimethylindazole (16 g, 62.717 mmol, 1 equiv) and bis(pinacolato)diboron (19.1 g, 75.260 mmol, 1.2 equiv) in dioxane (500 mL) was added KOAc (12.3 g, 125.434 mmol, 2 equiv) and Pd(dppf)Cl₂ (5.1 g, 6.272 mmol, 0.1 equiv) in portions at room temperature under nitrogen atmosphere. The resulting mixture was stirred for 3 h at 80° C. under nitrogen atmosphere, then cooled to room temperature and concentrated under reduced pressure to give a residue. The residue was purified by reverse flash chromatography (Condition 7, Gradient 1) to afford 6-methoxy-2,7-dimethyl-5-(4,4,5,5-tetramethyl-1,3,2-dioxaborolan-2-yl) indazole (9.3 g, 49%) as an oil. LCMS (ES, m/z): 303[M+H]⁺.

### Synthesis of Intermediate C71

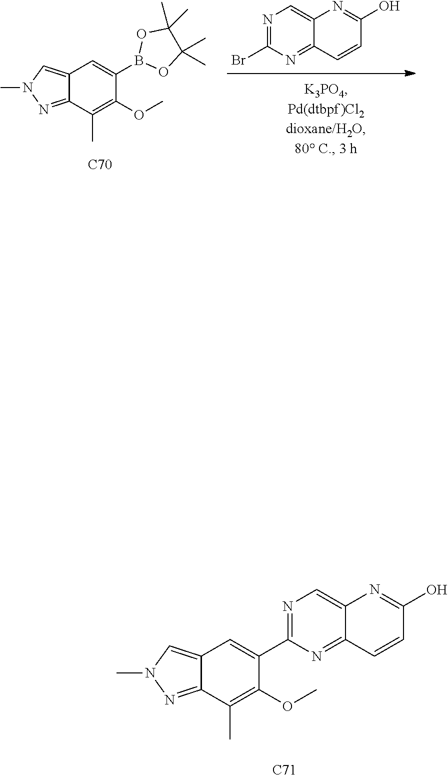

To a stirred mixture of 2-bromopyrido[3,2-d]pyrimidin-6-ol (5.5 g, 24.244 mmol, 1 equiv) and 6-methoxy-2,7-dimethyl-5-(4,4,5,5-tetramethyl-1,3,2-dioxaborolan-2-yl) indazole (7.3 g, 24.244 mmol, 1 equiv) in dioxane (200 mL) and water (20 mL) was added K₃PO₄ (15.4 g, 72.732 mmol, 3 equiv) and Pd(dppf)Cl₂ (1.8 g, 2.424 mmol, 0.1 equiv) in portions at room temperature under nitrogen atmosphere. The resulting mixture was stirred for 3 hr at 80° C. under nitrogen atmosphere, then cooled to room temperature and concentrated under reduced pressure to give a residue. The residue was purified by silica gel column chromatography, eluted with CH₂Cl₂/MeOH (10:1) to afford 2-(6-methoxy-2,7-dimethylindazol-5-yl)pyrido[3,2-d]pyrimidin-6-ol (4.7 g, 60%) as a solid. LCMS (ES, m/z): 322 [M+H]⁺.

### Synthesis of Intermediate C72

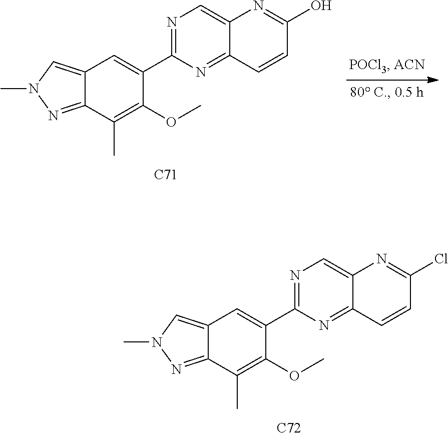 [SMILES: COc1c(-c2ncc3nc(O)ccc3n2)cc2cn(C)nc2c1C ; "C71" ; COc1c(-c2ncc3nc(Cl)ccc3n2)cc2cn(C)nc2c1C ; "C72"]

To a stirred solution of 2-(6-methoxy-2,7-dimethylindazol-5-yl)pyrido[3,2-d]pyrimidin-6-ol (4.7 g, 14.626 mmol, 1 equiv) in acetonitrile (100 mL) was added phosphorus oxychloride (22.4 g, 146.260 mmol, 10 equiv) dropwise at 0° C. The resulting mixture was stirred for 0.5 hr at 80° C. The resulting mixture was concentrated under reduced pressure, then basified to pH 8 with saturated NaHCO₃ (aq.) and extracted with ethyl acetate (3×100 mL). The organic layers were combined, washed with brine (1×100 mL), dried over anhydrous Na₂SO₄, and filtered. After filtration, the filtrate was concentrated under reduced pressure to give a residue. The residue was purified by silica gel column chromatography, eluted with PE/EA (1:10) to afford 5-{6-chloropyrido[3,2-d]pyrimidin-2-yl}-6-methoxy-2,7-dimethylindazole (1 g, 20%) as a solid. LCMS (ES, m/z): 340 [M+H]⁺.

### Synthesis of Intermediate C73

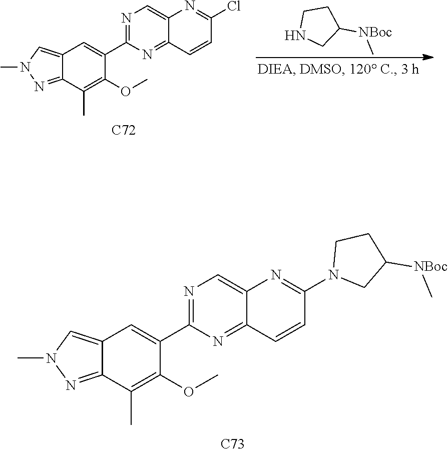

To a stirred mixture of 5-{6-chloropyrido[3,2-d]pyrimidin-2-yl}-6-methoxy-2,7-dimethylindazole (80 mg, 0.235 mmol, 1 equiv) and tert-butyl N-methyl-N-(pyrrolidin-3-yl)carbamate (94 mg, 0.470 mmol, 2 equiv) in DMSO (5 mL) was added DIEA (152 mg, 1.175 mmol, 5 equiv) dropwise at room temperature under nitrogen atmosphere. The resulting mixture was stirred for 3 hr at 120° C. under nitrogen atmosphere, then cooled to room temperature. The resulting mixture was purified by reverse flash chromatography (Condition 7, Gradient 2) to afford tert-butyl N-{1-[2-(6-methoxy-2,7-dimethylindazol-5-yl)pyrido[3,2-d]pyrimidin-6-yl]pyrrolidin-3-yl}-N-methylcarbamate (80 mg, 67%) as a solid. LCMS (ES, m/z): 504 [M+H]⁺.

### Synthesis of Compound 337

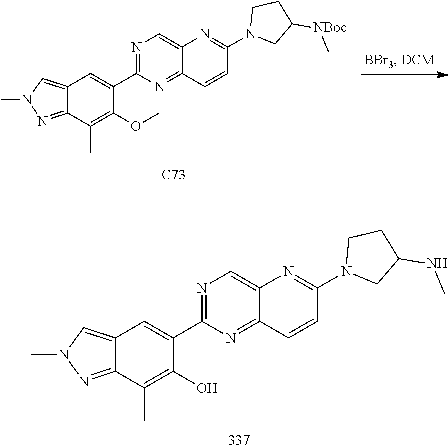

To a stirred solution of tert-butyl N-{1-[2-(6-methoxy-2,7-dimethylindazol-5-yl)pyrido[3,2-d]pyrimidin-6-yl]pyrrolidin-3-yl}-N-methylcarbamate (75 mg, 0.149 mmol, 1 equiv) in DCM (5 mL) was added BBr₃ (187 mg, 0.745 mmol, 5 equiv) dropwise at room temperature. The reaction mixture was stirred for 2 h at room temperature, then concentrated under reduced pressure to give a residue. The residue was quenched with MeOH at room temperature. The resulting mixture was concentrated under reduced pressure to give a residue. The residue was purified by reverse flash chromatography (Condition 3, Gradient 4) to afford 2,7-dimethyl-5-{6-[3-(methylamino)pyrrolidin-1-yl]pyrido[3,2-d]pyrimidin-2-yl}indazol-6-ol (20 mg, 34%) as a solid. LCMS (ES, m/z): 390 [M+H]⁺. ¹H NMR (400 MHz, DMSO-d₆) δ 13.32 (s, 1H), 9.26 (s, 1H), 8.84 (s, 1H), 8.39 (s, 1H), 8.17 (d, J=9.4 Hz, 1H), 7.42 (d, J=9.4 Hz, 1H), 4.14 (s, 3H), 3.77-3.60 (m, 3H), 3.40 (s, 3H), 2.40 (s, 3H), 2.33 (s, 3H), 2.17-2.08 (m, 1H), 1.90 (s, 1H).

### Example 131: Synthesis of Compound 338

### Synthesis of Intermediate C74

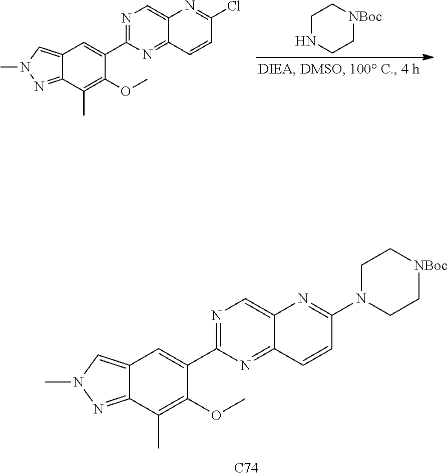 [SMILES: COc1c(-c2ncc3nc(Cl)ccc3n2)cc2cn(C)nc2c1C ; CC(C)(C)OC(=O)N1CCNCC1 ; COc1c(-c2ncc3nc(N4CCN(C(=O)OC(C)(C)C)CC4)ccc3n2)cc2cn(C)nc2c1C ; "C74"]

To a stirred mixture of 5-{6-chloropyrido[3,2-d]pyrimidin-2-yl}-6-methoxy-2,7-dimethylindazole (55 mg, 0.162 mmol, 1 equiv) and tert-butyl piperazine-1-carboxylate (91 mg, 0.486 mmol, 3 equiv) in DMSO (5 mL) was added DIEA (105 mg, 0.810 mmol, 5 equiv) dropwise at room temperature. The resulting mixture was stirred for 5 hr at 100° C., then cooled to room temperature. The resulting mixture was purified by reverse flash chromatography (Condition 7, Gradient 1) to afford tert-butyl 4-[2-(6-methoxy-2,7-dimethylindazol-5-yl)pyrido[3,2-d]pyrimidin-6-yl]piperazine-1-carboxylate (51 mg, 64%) as a solid. LCMS (ES, m/z): 490 [M+H]⁺.

### Synthesis of Compound 338

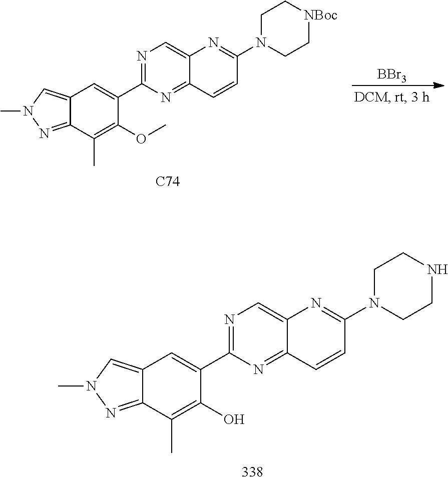 [SMILES: COc1c(-c2ncc3nc(N4CCN(C(=O)OC(C)(C)C)CC4)ccc3n2)cc2cn(C)nc2c1C ; "BBr3" ; "C74" ; Cc1c(O)c(-c2ncc3nc(N4CCNCC4)ccc3n2)cc2cn(C)nc12 ; "338"]

To a stirred solution of tert-butyl 4-[2-(6-methoxy-2,7-dimethylindazol-5-yl)pyrido[3,2-d]pyrimidin-6-yl]piperazine-1-carboxylate (25 mg, 0.051 mmol, 1 equiv) in DCM (5 mL) was added boron tribromide (128 mg, 0.510 mmol, 10 equiv) dropwise at room temperature. The resulting mixture was stirred for 3 hr at room temperature, then concentrated under reduced pressure to give a residue. The residue was quenched with methanol at room temperature. The resulting mixture was concentrated under reduced pressure to give a residue. The residue was purified by reverse flash chromatography (Condition 3, Gradient 4) to afford 2,7-dimethyl-5-[6-(piperazin-1-yl)pyrido[3,2-d]pyrimidin-2-yl]indazol-6-ol (13 mg, 68%) as a solid. LCMS (ES, m/z): 376 [M+H]⁺. ¹H NMR (300 MHz, DMSO-d₆) δ 13.29 (s, 1H), 9.25 (s, 1H), 8.85 (s, 1H), 8.40 (s, 1H), 8.18 (d, J=9.5 Hz, 1H), 7.77 (d, J=9.6 Hz, 1H), 4.14 (s, 3H), 3.73 (t, J=5.0 Hz, 4H), 2.83 (dd, J=6.2, 3.6 Hz, 4H), 2.40 (s, 3H).

### Example 132: Synthesis of Compound 342

### Synthesis of Intermediate C75

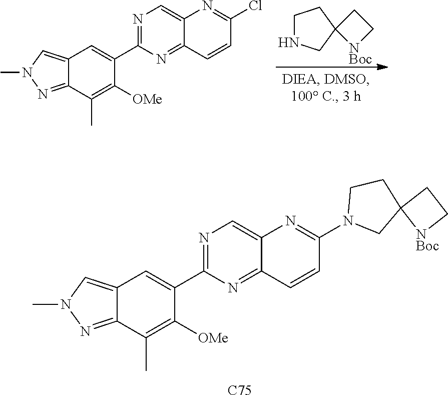

To a stirred mixture of 5-{6-chloropyrido[3,2-d]pyrimidin-2-yl}-6-methoxy-2,7-dimethylindazole (55 mg, 0.162 mmol, 1 equiv) and tert-butyl piperazine-1-carboxylate (91 mg, 0.486 mmol, 3 equiv) in DMSO (5 mL) was added DIEA (105 mg, 0.810 mmol, 5 equiv) dropwise at room temperature. The resulting mixture was stirred for 3 hr at 100° C., then cooled to room temperature. The resulting mixture was purified by reverse flash chromatography (Condition 7, Gradient 1) to afford tert-butyl 6-[2-(6-methoxy-2,7-dimethylindazol-5-yl)pyrido[3,2-d]pyrimidin-6-yl]-1,6-diazaspiro[3.4]octane-1-carboxylate (47 mg, 56%) as a solid. LCMS (ES, m/z): 516 [M+H]⁺.

### Synthesis of Compound 342

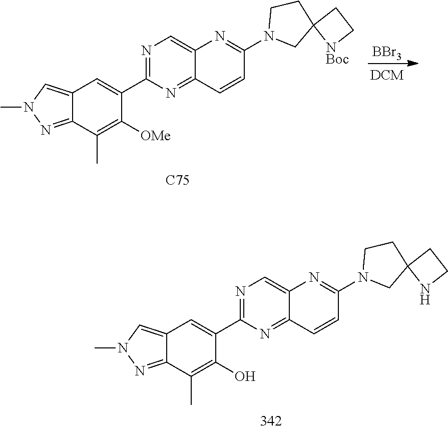

To a stirred solution of tert-butyl 6-[2-(6-methoxy-2,7-dimethylindazol-5-yl)pyrido[3,2-d]pyrimidin-6-yl]-1,6-diazaspiro[3.4]octane-1-carboxylate (25 mg, 0.048 mmol, 1 equiv) in DCM (5 mL) was added boron tribromide (122 mg, 0.480 mmol, 10 equiv) dropwise at room temperature. The resulting mixture was stirred for 3 h at room temperature, then concentrated under reduced pressure to give a residue. The residue was quenched with methanol at room temperature. The resulting mixture was concentrated under vacuum to give a residue. The residue was purified by reverse flash chromatography (Condition 3, Gradient 4) to afford 5-(6-{1,6-diazaspiro[3.4]octan-6-yl}pyrido[3,2-d]pyrimidin-2-yl)-2,7-dimethylindazol-6-ol (7 mg, 36%) as a solid. LCMS (ES, m/z): 402 [M+H]⁺. ¹H NMR (300 MHz, DMSO-d₆) δ 13.31 (s, 1H), 9.26 (s, 1H), 8.84 (s, 1H), 8.39 (s, 1H), 8.17 (d, J=9.3 Hz, 1H), 7.40 (d, J=9.4 Hz, 1H), 4.14 (s, 3H), 3.9 (s, 2H), 3.66 (d, J=13.4 Hz, 4H), 2.40 (s, 3H), 2.19 (s, 2H). 2.17 (s, 2H).

### Example 133: Synthesis of Compound 331, 332, and 393

### Synthesis of Intermediate C76

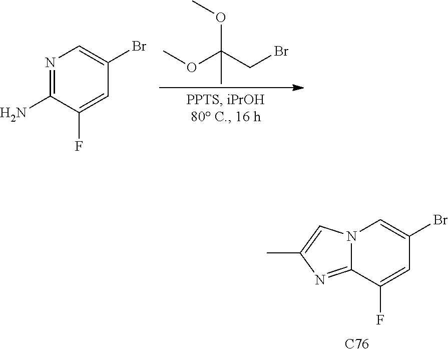

To a stirred mixture of 5-bromo-3-fluoropyridin-2-amine (2 g, 10.471 mmol, 1.00 equiv) and 1-bromo-2,2-dimethoxypropane (2.3 g, 12.565 mmol, 1.2 equiv) in i-PrOH (20 mL) was added PPTS (0.26 g, 1.047 mmol, 0.1 equiv) in portions at room temperature. The resulting mixture was stirred for 16 h at 80° C., then cooled to room temperature. The resulting mixture was diluted with water (20 mL) and adjusted to pH 9 with saturated aqueous NaHCO₃. A precipitate formed that was collected by filtration and dried in vacuum to afford 6-bromo-8-fluoro-2-methyl-1H,8aH-imidazo[1,2-a]pyridine (1.0 g, 41%) as a solid. LCMS (ES, m/z): 229 [M+H]⁺.

### Synthesis of Intermediate C77

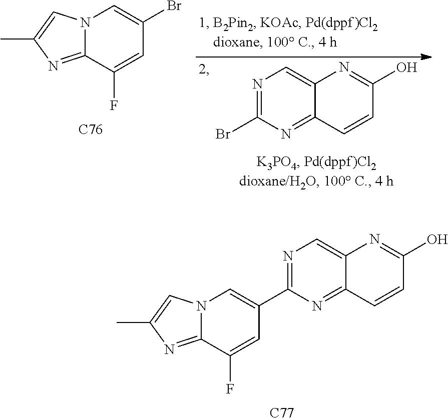 [SMILES: "C76" ; Cc1cn2cc(Br)cc(F)c2n1 ; Oc1ccc2nc(Br)ncc2n1 ; Cc1cn2cc(-c3ncc4nc(O)ccc4n3)cc(F)c2n1 ; "C77"]

To a stirred mixture of 6-bromo-8-fluoro-2-methylimidazo[1,2-a]pyridine (200 mg, 0.873 mmol, 1 equiv) and bis(pinacolato)diboron (244 mg, 0.960 mmol, 1.1 equiv) in dioxane (5 mL) was added KOAc (171 mg, 1.746 mmol, 2 equiv) and Pd(dppf)Cl₂ (64 mg, 0.087 mmol, 0.1 equiv) in portions at room temperature under nitrogen atmosphere. The resulting mixture was stirred for 4 hr at 100° C. under nitrogen atmosphere, then cooled to room temperature. To the resulting mixture was added 2-bromopyrido[3,2-d]pyrimidin-6-ol (237 mg, 1.048 mmol, 1.2 equiv), K₃PO₄ (371 mg, 1.746 mmol, 2 equiv), water (1 mL), and Pd(dppf)Cl₂ (64 mg, 0.087 mmol, 0.1 equiv) in portions at room temperature. The resulting mixture was stirred for an additional 4 hr at 100° C., then cooled to room temperature. The resulting mixture was diluted with ethyl acetate (10 mL) and water (10 mL). The resulting mixture was filtered, and the filter cake was washed with ethyl acetate (3×5 mL). The filtrate was concentrated under reduced pressure to afford 2-{8-fluoro-2-methylimidazo[1,2-a]pyridin-6-yl}pyrido[3,2-d]pyrimidin-6-ol (250 mg, 97%) as a solid. LCMS (ES, m/z): 296 [M+H]⁺.

### Synthesis of Intermediate C78

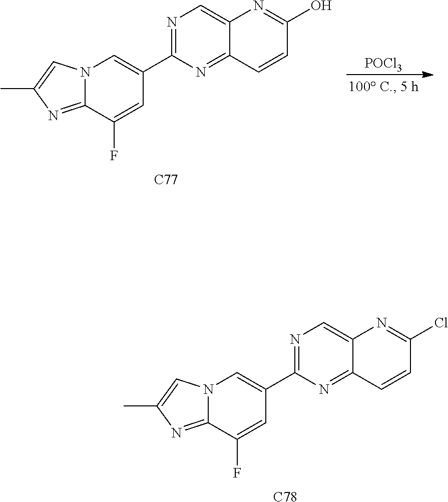

A solution of 2-{8-fluoro-2-methylimidazo[1,2-a]pyridin-6-yl}pyrido[3,2-d]pyrimidin-6-ol (240 mg, 0.813 mmol, 1 equiv) in phosphorus oxychloride (10 mL) was stirred for 5 h at 110° C., then cooled to room temperature. The resulting mixture was concentrated under vacuum to give a residue. The residue was basified to pH 8 with saturated NaHCO₃ (aq.) and extracted with ethyl acetate (3×10 mL). The organic layers were combined, washed with brine (1×10 mL), dried over anhydrous NaSO₄, and filtered. After filtration, the filtrate was concentrated under reduced pressure to afford 6-{6-chloropyrido[3,2-d]pyrimidin-2-yl}-8-fluoro-2-methylimidazo[1,2-a]pyridine (120 mg, 47%) as a solid. LCMS (ES, m/z): 314 [M+H]⁺.

### Synthesis of Intermediate C79

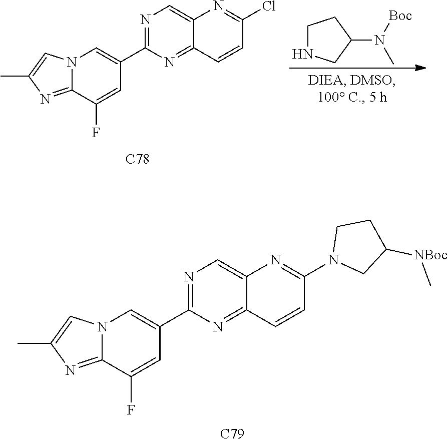

To a stirred mixture of 6-{6-chloropyrido[3,2-d]pyrimidin-2-yl}-8-fluoro-2-methylimidazo[1,2-a]pyridine (45 mg, 0.143 mmol, 1 equiv) and tert-butyl N-methyl-N-(pyrrolidin-3-yl)carbamate (58 mg, 0.286 mmol, 2 equiv) in DMSO (5 mL) was added DIEA (93 mg, 0.715 mmol, 5 equiv) dropwise at room temperature. The resulting mixture was stirred for 5 h at 100° C., then cooled to room temperature and concentrated under reduced pressure to give a residue. The residue was purified by reverse flash chromatography (Condition 3, Gradient 5) to afford tert-butyl N-[1-(2-{8-fluoro-2-methylimidazo[1,2-a]pyridin-6-yl}pyrido[3,2-d]pyrimidin-6-yl)pyrrolidin-3-yl]-N-methylcarbamate (38 mg, 55%) as a solid. LCMS (ES, m/z): 478 [M+H]⁺.

### Synthesis of Compound 393

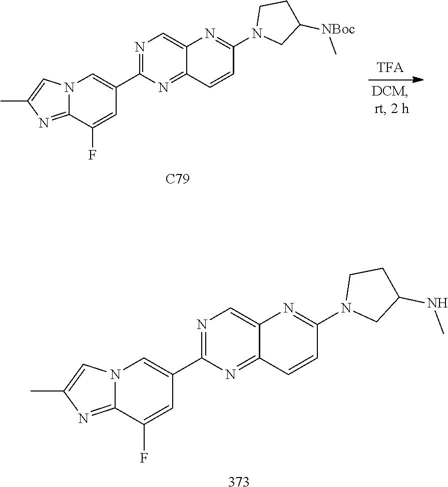

A mixture of tert-butyl N-[1-(2-{8-fluoro-2-methylimidazo[1,2-a]pyridin-6-yl}pyrido[3,2-d]pyrimidin-6-yl)pyrrolidin-3-yl]-N-methylcarbamate (33 mg, 0.069 mmol, 1 equiv) in TFA (1 mL) and DCM (1 mL) was stirred for 2 hr at room temperature. The resulting mixture was concentrated under reduced pressure to give a residue. The residue was basified to pH 8 with 7 M NH₃(g) in methanol, then concentrated under vacuum to give a residue. The residue was purified by reverse flash chromatography (Condition 3, Gradient 5) to afford 1-(2-{8-fluoro-2-methylimidazo[1,2-a]pyridin-6-yl}pyrido[3,2-d]pyrimidin-6-yl)-N-methylpyrrolidin-3-amine (20 mg, 77%) as a solid. LCMS (ES, m/z): 378 [M+H]⁺. ¹H NMR (300 MHz, DMSO-d₆) δ 9.37 (s, 1H), 9.17 (s, 1H), 8.07 (d, J=9.3 Hz, 1H), 8.01 (d, J=3.1 Hz, 1H), 7.90 (d, J=12.6 Hz, 1H), 7.38 (d, J=9.4 Hz, 1H), 3.74-3.59 (m, 3H), 3.42 (s, 3H), 2.37 (d, J=11.4 Hz, 6H), 2.12 (s, 1H), 1.90 (s, 1H).

### Synthesis of Compound 331 and 332

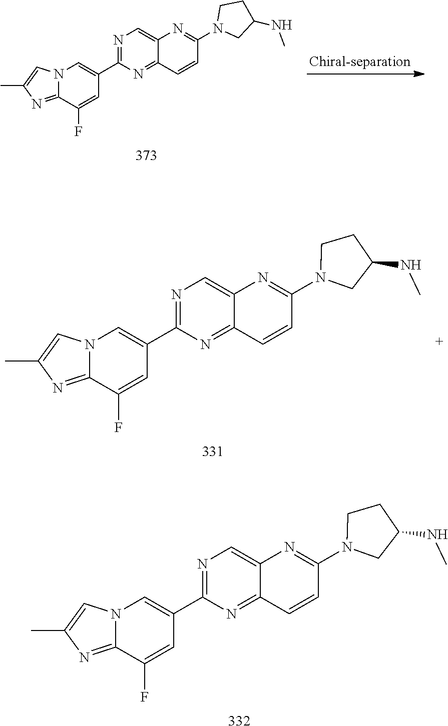

6-{2,8-dimethylimidazo[1,2-b]pyridazin-6-yl}-8-fluoro-2-(pyrrolidin-3-yl)isoquinolin-1-one (16.7 mg) was purified by chiral-HPLC (Condition 3, Gradient 2) to afford (R)-1-(2-(8-fluoro-2-methylimidazo[1,2-a]pyridin-6-yl)pyrido[3,2-d]pyrimidin-6-yl)-N-methylpyrrolidin-3-amine (4.7 mg, 28%) and (S)-1-(2-(8-fluoro-2-methylimidazo[1,2-a]pyridin-6-yl)pyrido[3,2-d]pyrimidin-6-yl)-N-methylpyrrolidin-3-amine (4.7 mg, 28%) as solids. Compound 331 RT=2.359 min. LCMS (ES, m/z): 378 [M+H]⁺. ¹H NMR (300 MHz, DMSO-d₆) δ 9.37 (s, 1H), 9.17 (s, 1H), 8.07 (d, J=9.3 Hz, 1H), 8.01 (d, J=3.1 Hz, 1H), 7.90 (d, J=12.6 Hz, 1H), 7.38 (d, J=9.4 Hz, 1H), 3.74-3.59 (m, 3H), 3.42 (s, 3H), 2.37 (d, J=11.4 Hz, 6H), 2.12 (s, 1H), 1.90 (s, 1H). Compound 332RT=3.251. LCMS (ES, m/z): 378 [M+H]⁺. ¹H NMR (300 MHz, DMSO-d₆) δ 9.37 (s, 1H), 9.17 (s, 1H), 8.07 (d, J=9.3 Hz, 1H), 8.01 (d, J=3.1 Hz, 1H), 7.90 (d, J=12.6 Hz, 1H), 7.38 (d, J=9.4 Hz, 1H), 3.74-3.59 (m, 3H), 3.42 (s, 3H), 2.37 (d, J=11.4 Hz, 6H), 2.12 (s, 1H), 1.90 (s, 1H).

### Example 134: Synthesis of Compound 361

### Synthesis of Intermediate C80

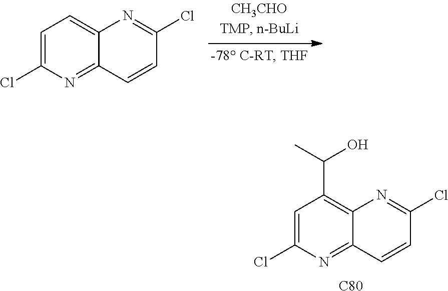

A solution of 2,6-dichloro-1,5-naphthyridine (3.15 g, 15.827 mmol, 1 equiv) in THE (60 mL) was treated with 2,2,6,6-Tetramethylpiperidinylmagnesium chloride lithium chloride complex solution in (1M) THE (19.18 g, 79.135 mmol, 5 equiv) for 0.5 h at −78° C. under nitrogen atmosphere followed by the addition of acetaldehyde (1.39 g, 31.654 mmol, 2 equiv) dropwise at −78° C. The mixture was allowed to warm up to room temperature. The resulting mixture was extracted with water and ethyl acetate. The combined organic layers were dried over anhydrous Na₂SO₄. After filtration, the filtrate was concentrated under reduced pressure. The residue was purified by silica gel column chromatography, eluted with PE:EA=3:1 to afford 1-(2,6-dichloro-1,5-naphthyridin-4-yl)ethanol (0.72 g, 18%) as an oil. LCMS (ES, m/z): 243 [M+H]⁺

### Synthesis of Intermediate C81

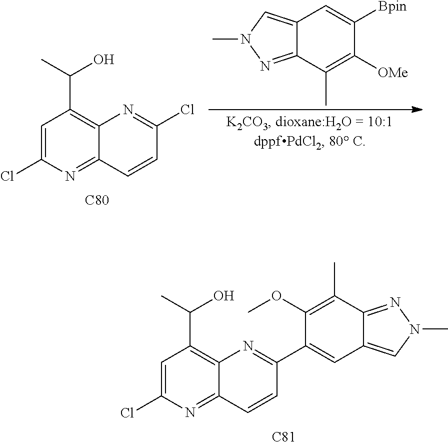

A mixture of 1-(2,6-dichloro-1,5-naphthyridin-4-yl) ethanol (190 mg, 0.782 mmol, 1 equiv), 6-methoxy-2,7-dimethyl-5-(4,4,5,5-tetramethyl-1,3,2-dioxaborolan-2-yl) indazole (259.80 mg, 0.860 mmol, 1.1 equi v), Pd(dppf)Cl₂ (5.72 mg, 0.008 mmol, 0.01 equiv) and K₂CO₃ (216.04 mg, 1.564 mmol, 2 equiv) in dioxane (4 mL) and H₂O (0.4 mL) was stirred for 1 h at 80° C. under Nitrogen atmosphere. The resulting mixture was concentrated under reduced pressure. The residue was purified by silica gel column chromatography, eluted with PE:EA=3:1 to afford 1-[2-chloro-6-(6-methoxy-2,7-dimethylindazol-5-yl)-1,5-naphthyridin-4-yl]ethanol (93 mg, 31%) as a solid. LCMS (ES, m/z): 383 [M+H]⁺

### Synthesis of Intermediate C82

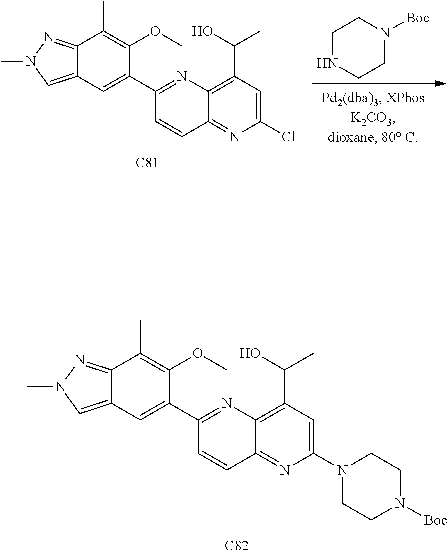 [SMILES: COc1c(-c2ccc3nc(Cl)cc(C(C)O)c3n2)cc2cn(C)nc2c1C ; CC(C)(C)OC(=O)N1CCNCC1 ; "C81" ; COc1c(-c2ccc3nc(N4CCN(C(=O)OC(C)(C)C)CC4)cc(C(C)O)c3n2)cc2cn(C)nc2c1C ; "C82"]

A mixture of 1-[2-chloro-6-(6-methoxy-2,7-dimethylindazol-5-yl)-1,5-naphthyridin-4-yl]ethanol (90 mg, 0.235 mmol, 1 equiv), 1-[2-chloro-6-(6-methoxy-2,7-dimethylindazol-5-yl)-1,5-naphthyridin-4-yl]ethanol (90 mg, 0.235 mmol, 1 equiv), tert-butyl piperazine-1-carboxylate (52.54 mg, 0.282 mmol, 1.2 equiv), Pd₂(dba)₃ (2.15 mg, 0.002 mmol, 0.01 equiv) and K₂CO₃ (97.47 mg, 0.705 mmol, 3 equiv) in dioxane (2 mL) was stirred for 5 h at 80° C. under Nitrogen atmosphere. The resulting mixture was concentrated under reduced pressure. The residue was purified by silica gel column chromatography, eluted with PE:EA=1:1 to afford tert-butyl-4-[4-(1-hydroxyethyl)-6-(6-methoxy-2,7-dimethylindazol-5-yl)-1,5-naphthyridin-2-yl]piperazine-1-carboxylate (60 mg, 47%) as a solid. LCMS (ES, m/z): 533 [M+H]⁺

### Synthesis of Compound 361

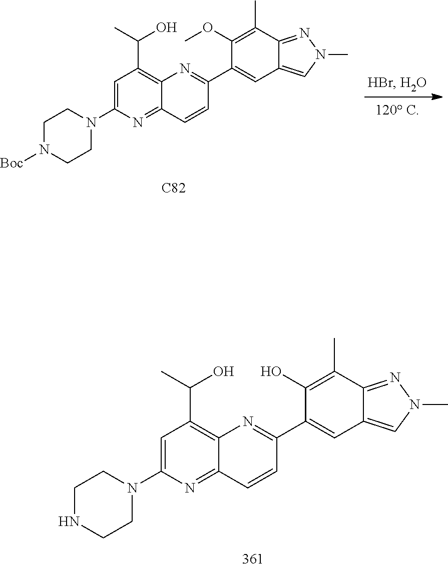 [SMILES: COc1c(-c2ccc3nc(N4CCN(C(=O)OC(C)(C)C)CC4)cc(C(C)O)c3n2)cc2cn(C)nc2c1C ; "C82" ; Cc1c(O)c(-c2ccc3nc(N4CCNCC4)cc(C(C)O)c3n2)cc2cn(C)nc12 ; "361"]

A solution of tert-butyl 4-[4-(1-hydroxyethyl)-6-(6-methoxy-2,7-dimethylindazol-5-yl)-1,5-naphthyridin-2-yl]piperazine-1-carboxylate (60 mg, 0.113 mmol, 1 equiv) in H₂O (2 mL) was treated with HBr (0.2 mL, 6.847 mmol, 60.78 equiv) for 1 hr at 120° C. The residue was purified by Prep-TLC (Condition 1, Gradient 1) to afford 5-[8-(1-hydroxyethyl)-6-(piperazin-1-yl)-1,5-naphthyridin-2-yl]-2,7-dimethylindazol-6-ol (1.1 mg, 2%) as a solid. LCMS (ES, m/z): 419 [M+H]^{+ 1}H NMR (300 MHz, DMSO-d₆) δ 8.56-8.43 (m, 1H), 8.36 (s, 1H), 8.15 (d, J=9.1 Hz, 1H), 7.60 (s, 1H), 5.54 (d, J=5.5 Hz, 1H), 4.14 (s, 3H), 3.96 (d, J=6.2 Hz, 4H), 2.39 (s, 7H), 1.54 (d, J=6.0 Hz, 3H).

### Example 135: Synthesis of Compounds 318, 319, and 320

### Synthesis of Intermediate C83

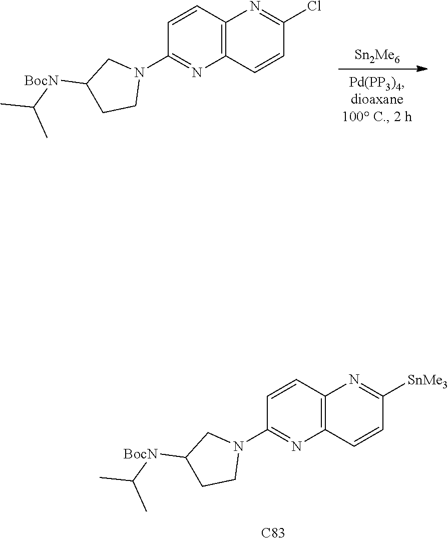

To a stirred solution of tert-butyl N-[1-(6-chloro-1,5-naphthyridin-2-yl) pyrrolidin-3-yl]-N-isopropyl carbamate (1 g, 2.558 mmol, 1 equiv) and Pd(PPh₃)₄(0.59 g, 0.512 mmol, 0.2 equiv) in 1,4-dioxane (20 mL) were added hexamethyldistannan-1-ylidyne (1.68 g, 5.116 mmol, 2 equiv) dropwise for 2 hr at 100° C. under N₂ atmosphere. The mixture was allowed to cool down to room temperature. The reaction was quenched with water at 0° C. The resulting mixture was extracted with ethyl acetate (2×40 mL). The combined organic layers were washed with water (3×40 mL), dried over anhydrous Na₂SO₄. After filtration, the filtrate was concentrated under reduced pressure. This resulted in tert-butyl N-isopropyl-N-{1-[6-(trimethyl stannyl)-1,5-naphthyridin-2-yl]pyrrolidin-3-yl}carbamate (1.2 g, 72%) as a solid. LCMS (ES, m/z): 521 [M+H]

### Synthesis of Intermediate C84

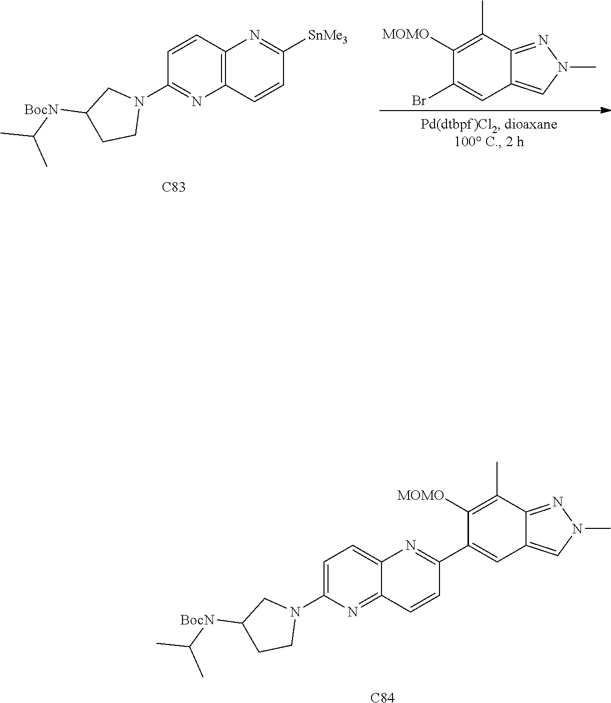

To a stirred solution of 5-bromo-6-(methoxymethoxy)-2,7-dimethylindazole (494.20 mg, 1.733 mmol, 1 equiv) and Pd(DtBPF)Cl₂ (225.92 mg, 0.347 mmol, 0.2 equiv) in dioxane (20 mL) was added tert-butyl N-isopropyl-N-{1-[6-(trimethylstannyl)-1,5-naphthyridin-2-yl]pyrrolidin-3-yl}carbamate (900 mg, 1.733 mmol, 1 equiv) dropwise for 2 hr at 100° C. under N₂ atmosphere. The mixture was allowed to cool down to room temperature. The resulting mixture was diluted with water (30 mL). The resulting mixture was extracted with ethylacetate (3×40 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (3:1) to afford tert-butyl N-isopropyl-N-(1-{6-[6-(methoxymethoxy)-2,7-dimethylindazol-5-yl]-1,5-naphthyridin-2-yl}pyrrolidin-3-yl)carbamate (300 mg, 30%) as a solid. LCMS (ES, m/z): 561 [M+H]

### Synthesis of Compound 318

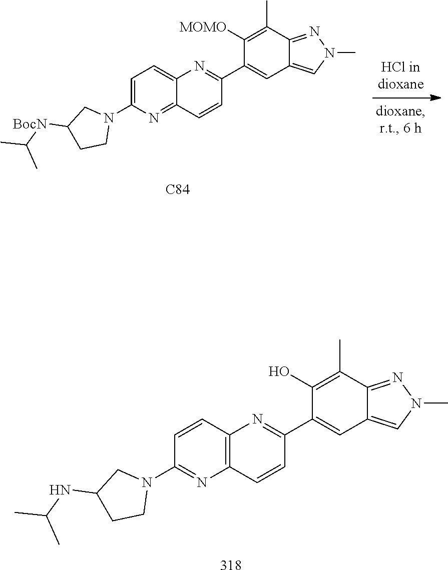

To a stirred solution of 5-{6-[3-(isopropylamino) pyrrolidin-1-yl]-1,5-naphthyridin-2-yl}-2,7-dimethylindazol-6-ol (260 mg, 0.624 mmol, 1 equiv) in 1,4-dioxane (30 mL) were added HCl(gas) in 1,4-dioxane (20 mL, 658.238 mmol, 1419.51 equiv) in portions for 6 h at room temperature. The resulting mixture was concentrated under reduced pressure. The crude product was purified by Prep-HPLC (Condition 1, Gradient 28) to afford 5-{6-[3-(isopropylamino)pyrrolidin-1-yl]-1,5-naphthyridin-2-yl}-2,7-dimethylindazol-6-ol (42 mg, 21%) as a solid. LCMS (ES, m/z): 417 [M+H]

### Synthesis of Compounds 319 and 32—

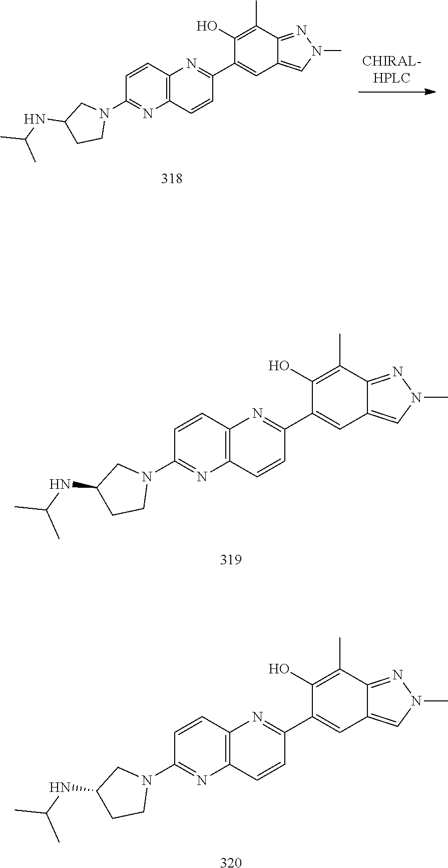

The crude product (5-{6-[3-(isopropylamino)pyrrolidin-1-yl]-1,5-naphthyridin-2-yl}-2,7-dimethylindazol-6-ol (100 mg, 0.240 mmol, 1 equiv) was purified by CHIRAL-HPLC (Condition 4, Gradient 3) to afford (48.5 mg, 48H) and (49.5 mg, 49)) as solid.

### Analysis Data

| Compound Structure and No. | Analysis data |
|---|---|
| 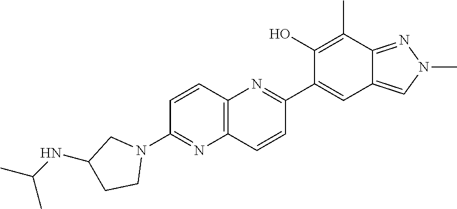 [SMILES: Cc1c(O)c(-c2ccc3nc(N4CCC(NC(C)C)C4)ccc3n2)cc2cn(C)nc12] | LCMS (ES, m/z): 417 [M + H] ¹H NMR (400 MHz, DMSO-d6) δ 14.32 (s, 1H), 8.43-8.36 (m, 2H), 8.32 (s, 1H), 8.12 (d, J = 9.3 Hz, 1H), 8.06 (d, J = 9.1 Hz, 1H), 7.15 (d, J = 9.3 Hz, 1H), 4.13 (s, 3H), 3.78 (d, J = 8.9 Hz, 1H), 3.70 (s, 1H), 3.52 (s, 2H), 3.32 (s, 1H), 2.86 (p, J = 6.2 Hz, 1H), 2.38 (s, 3H), 2.16 (tt, J = 12.4, 5.6 Hz, 1H), 1.80 (s, 2H), 1.02 (dd, J = 6.2, 4.5 Hz, 6H). |
| 318 | |
| 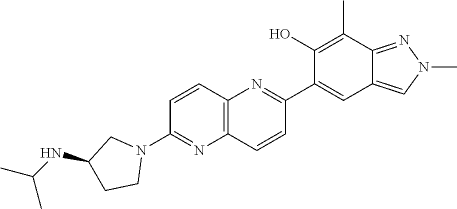 [SMILES: Cc1c(O)c(-c2ccc3nc(N4CC[C@@H](NC(C)C)C4)ccc3n2)cc2cn(C)nc12] | LCMS (ES, m/z): 417 [M + H] ¹H NMR (400 MHz, DMSO-d6) δ 14.32 (s, 1H), 8.42-8.36 (m, 2H), 8.32 (s, 1H), 8.12 (dd, J = 9.3, 0.8 Hz, 1H), 8.05 (dd, J = 9.1, 0.8 Hz, 1H), 7.15 (d, J = 9.3 Hz, 1H), 4.13 (s, 3H), 3.79 (dd, J = 10.2, 6.0 Hz, 1H), 3.70 (s, 1H), 3.53 (s, 2H), 3.32 (s, 1H), 2.86 (p, J = 6.2 Hz, 1H), 2.38 (s, 3H), 2.16 (dq, J = 12.5, 6.2 Hz, 1H), 1.81 (dt, J = 12.6, 7.1 Hz, 1H), 1.72 (s, 1H), 1.02 (dd, J = 6.2, 4.6 Hz, 6H). |
| 319 | |
| 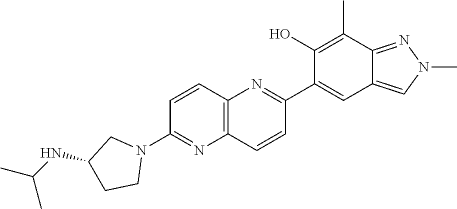 [SMILES: Cc1c(O)c(-c2ccc3nc(N4CC[C@H](NC(C)C)C4)ccc3n2)cc2cn(C)nc12] | LCMS (ES, m/z): 417 [M + H] ¹H NMR (400 MHz, DMSO-d6) δ 14.32 (s, 1H), 8.43-8.36 (m, 2H), 8.32 (s, 1H), 8.12 (dd, J = 9.3, 0.8 Hz, 1H), 8.09-8.02 (m, 1H), 7.15 (d, J = 9.3 Hz, 1H), 4.13 (s, 3H), 3.84-3.75 (m, 1H), 3.70 (s, 1H), 3.53 (dd, J = 11.2, 7.1 Hz, 2H), 3.32 (s, 1H), 2.86 (p, J = 6.3 Hz, 1H), 2.38 (s, 3H), 2.16 (dq, J = 12.5, 6.3 Hz, 1H), 1.81 (dd, J = 12.4, 6.9 Hz, 1H), 1.76 (s, 1H), 1.02 (dd, J = 6.2, 4.5 Hz, 6H). |
| 320 | |

### Example 136: Synthesis of Compounds 328, 329, and 330

### Synthesis of Intermediate C85

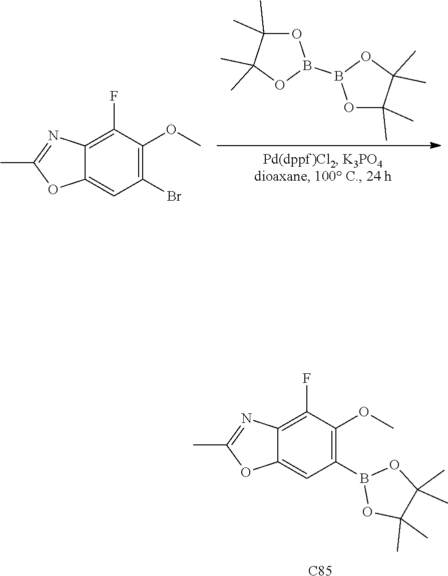

To a stirred solution of 6-bromo-4-fluoro-5-methoxy-2-methyl-1,3-benzoxazole (500 mg, 1.923 mmol, 1 equiv) and Pd(dppf)Cl₂·CH₂Cl₂ (313.24 mg, 0.385 mmol, 0.2 equiv) in 1,4-dioxane (20 mL) were added K₃PO₄ (1224.31 mg, 5.769 mmol, 3 equiv) and bis(pinacolato)diboron (976.46 mg, 3.846 mmol, 2 equiv) in portions at room temperature. A solution was stirred for 24 hr at 100° C. under N₂ atmosphere. The mixture was allowed to cool down to room temperature. The resulting mixture was extracted with ethyl acetate (2×30 mL). The combined organic layers were washed with water (3×30 mL), dried over anhydrous Na₂SO₄. After filtration, the filtrate was concentrated under reduced pressure. This resulted in 4-fluoro-5-methoxy-2-methyl-6-(4,4,5,5-tetramethyl-1,3,2-dioxaborolan-2-yl)-1,3-benzoxazole (950 mg, 72%) as an oil. LCMS (ES, m/z): 308 [M+H]

### Synthesis of Intermediate C86

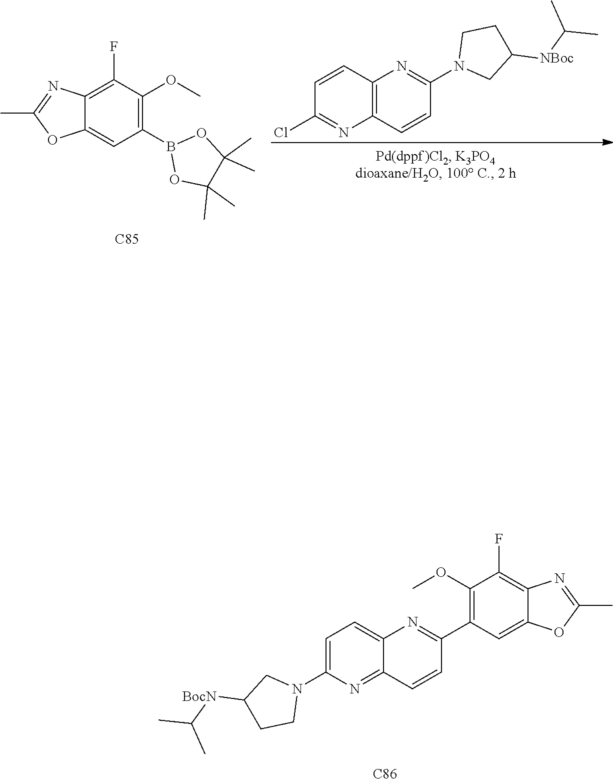

To a stirred solution of tert-butyl N-[1-(6-chloro-1,5-naphthyridin-2-yl)pyrrolidin-3-yl]-N-isopropylc arbamate (900 mg, 2.302 mmol, 1 equiv) and 4-fluoro-5-methoxy-2-methyl-6-(4,4,5,5-tetramethyl-1,3,2-dioxaborolan-2-yl)-1,3-benzoxazole (1.06 g, 3.453 mmol, 1.5 equiv) and Pd(dppf)Cl₂·CH₂Cl₂ (375.10 mg, 0.460 mmol, 0.2 equiv) in 1,4-dioxane (16 mL) and H₂O (4 mL) were added K₃PO₄ (1.46 g, 6.906 mmol, 3 equiv) dropwise at room temperature. A mixture was stirred for 2 hr at 100° C. under N₂ atmosphere. The mixture was allowed to cool down to room temperature. The reaction was quenched with water at room temperature. The resulting mixture was extracted with ethyl acetate (3×30 mL). The combined organic layers were washed with water (1×30 mL), dried over anhydrous Na₂SO₄. After filtration, the filtrate was concentrated under reduced pressure. The residue was purified by silica gel column chromatography, eluted with PE/EA (1:1) to afford tert-butyl N-{1-[6-(4-fluoro-5-methoxy-2-methyl-1,3-benzoxazol-6-yl)-1,5-naphthyridin-2-yl]pyrrolidin-3-yl}-N-isopropylcarbamate (420 mg, 34%) as a solid. LCMS (ES, m/z): 536 [M+H]

### Synthesis of Compound 328

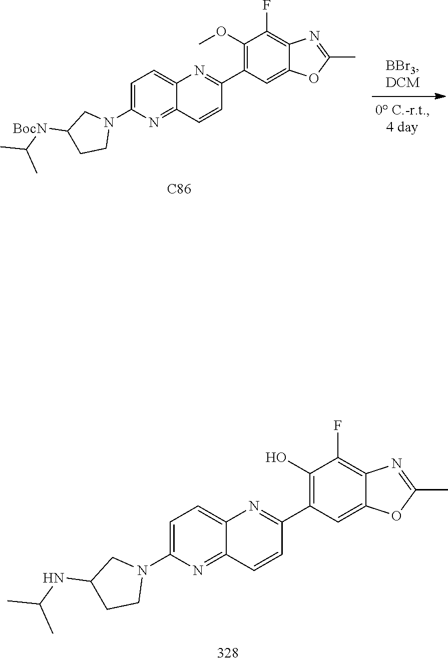

To a stirred solution of 4-fluoro-6-{6-[3-(isopropylamino)pyrrolidin-1-yl]-1,5-naphthyridin-2-yl}-2-methyl-1,3-benzoxazol-5-ol (380 mg, 0.902 mmol, 1 equiv) in DCM (30 mL) were added BBr₃ (1.12 g, 4.510 mmol, 5 equiv) in portions for 4 days at room temperature. The reaction was quenched by the addition of methanol (10 mL) at 0° C. The resulting mixture was concentrated under reduced pressure. The resulting mixture was extracted with ethyl acetate (1×30 mL). The combined organic layers were washed with water (3×20 mL), dried over anhydrous Na₂SO₄. After filtration, the filtrate was concentrated under reduced pressure. The crude product was purified by Prep-HPLC (Condition 1, Gradient 24) to afford tert-butyl N-{1-[6-(4-fluoro-5-methoxy-2-methyl-1,3-benzoxazol-6-yl)-1,5-naphthyridin-2-yl]pyrrolidin-3-yl}-N-isopropylcarbamate (33.7 mg, 6%) as a solid. LCMS (ES, m/z): 422 [M+H]

### Synthesis of Compounds 329 and 330

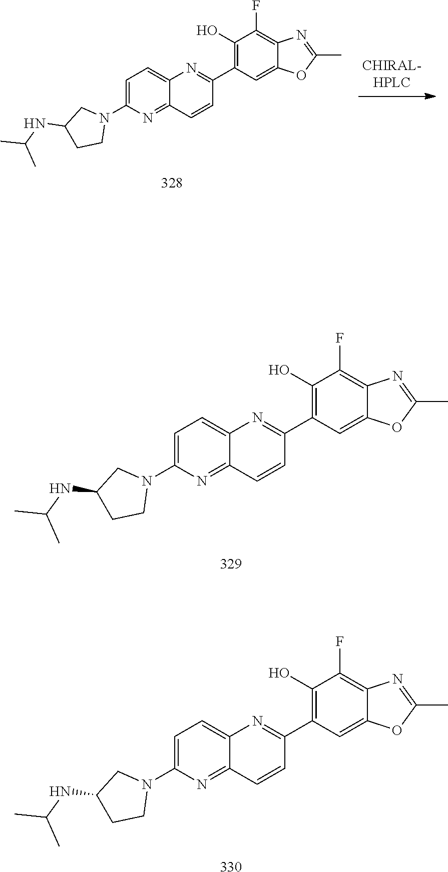

The crude product (4-fluoro-6-{6-[3-(isopropylamino) pyrrolidin-1-yl]-1,5-naphthyridin-2-yl}-2-methyl-1,3-benzoxazol-5-ol (120 mg, 0.285 mmol, 1 equiv) was purified by Prep-HPLC (Condition 4, Gradient 4) to afford (48.1 mg, 40%) and (48.3 mg, 40%) as solids.

### Analysis Data

| Compound Structure and No. | Analysis data |
|---|---|
| 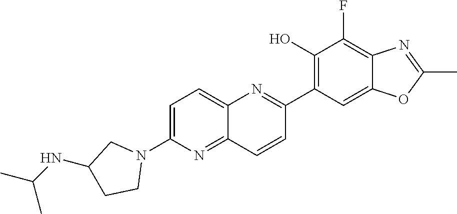 [SMILES: Cc1nc2c(F)c(O)c(-c3ccc4nc(N5CCC(NC(C)C)C5)ccc4n3)cc2o1] | LCMS (ES, m/z): 422 [M + H] ¹H NMR (400 MHz, DMSO-d₆) δ 15.07 (s, 1H), 8.44 (d, J = 9.2 Hz, 1H), 8.33 (d, J = 1.4 Hz, 1H), 8.16 (d, J = 9.3 Hz, 1H), 8.11 (d, J = 9.0 Hz, 1H), 7.19 (d, J = 9.3 Hz, 1H), 3.80 (s, 1H), 3.71 (s, 1H), 3.54 (s, 2H), 3.32 (s, 1H), 2.91-2.83 (m, 1H), 2.64 (s, 3H), 2.16 (dd, J = 12.0, 6.2 Hz, 1H), 1.82 (s, 2H), 1.03 (dd, J = 6.2, 4.5 Hz, 6H). FNMR-: ¹⁹F NMR (376 MHz, DMSO-d₆) δ −151.11. |
| 328 | |
| 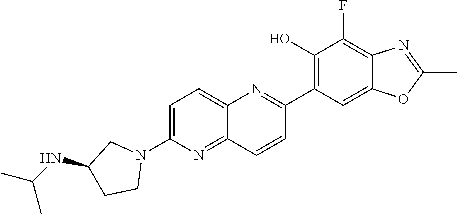 [SMILES: Cc1nc2c(F)c(O)c(-c3ccc4nc(N5CC[C@@H](NC(C)C)C5)ccc4n3)cc2o1] | LCMS (ES, m/z): 422 [M + H] ¹H NMR (400 MHz, DMSO-d₆) δ 15.07 (s, 1H), 8.43 (d, J = 9.2 Hz, 1H), 8.34-8.29 (m, 1H), 8.15 (d, J = 9.3 Hz, 2H), 7.18 (d, J = 9.3 Hz, 1H), 3.79 (s, 1H), 3.71 (s, 1H), 3.54 (d, J = 10.1 Hz, 2H), 3.32 (s, 1H), 2.87 (p, J = 6.2 Hz, 1H), 2.64 (s, 3H), 2.17 (dt, J = 12.5, 6.4 Hz, 1H), 1.82 (s, 2H), 1.03 (dd, J = 6.2, 4.5 Hz, 6H). FNMR: ¹⁹F NMR (376 MHz, DMSO-d₆) δ −151.11. |
| 329 | |
| 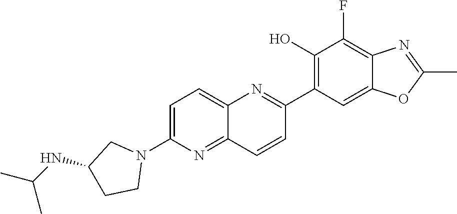 [SMILES: Cc1nc2c(F)c(O)c(-c3ccc4nc(N5CC[C@H](NC(C)C)C5)ccc4n3)cc2o1] | LCMS (ES, m/z): 422 [M + H] ¹H NMR (400 MHz, DMSO-d₆) δ 15.07 (s, 1H), 8.42 (d, J = 9.2 Hz, 1H), 8.30 (d, J = 1.4 Hz, 1H), 8.14 (d, J = 9.3 Hz, 1H), 8.09 (d, J = 9.1 Hz, 1H), 7.17 (d, J = 9.3 Hz, 1H), 3.78 (d, J = 8.2 Hz, 1H), 3.70 (s, 1H), 3.53 (q, J = 7.0, 5.5 Hz, 2H), 3.33 (s, 1H), 2.87 (hept, J = 6.2 Hz, 1H), 2.64 (s, 3H), 2.16 (dq, J = 12.6, 6.5 Hz, 1H), 1.81 (s, 2H), 1.03 (dd, J = 6.2, 4.6 Hz, 6H). FNMR: ¹⁹F NMR (376 MHz, DMSO-d₆) δ −151.10. |
| 330 | |

### Example 137: Synthesis of Compounds 401 and 402

### Synthesis of Intermediate C87

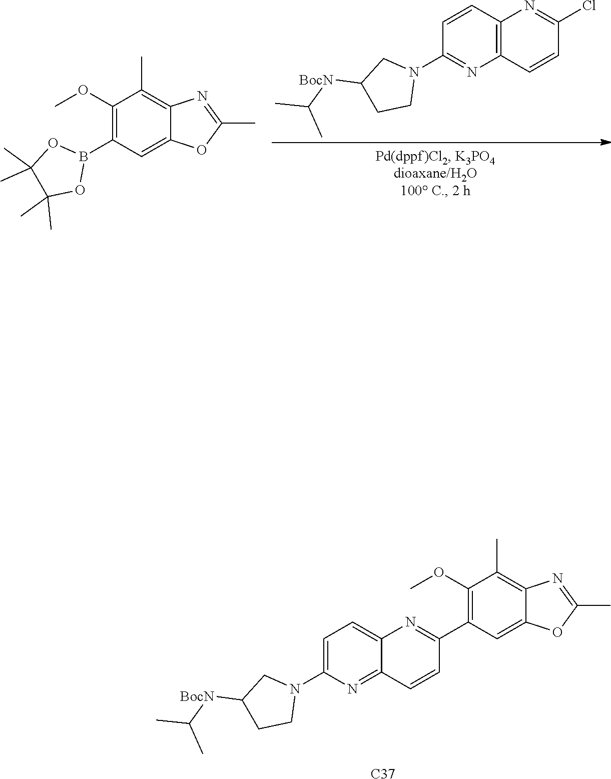

To a stirred solution of 5-methoxy-2,4-dimethyl-6-(4,4,5,5-tetramethyl-1,3,2-dioxaborolan-2-yl)-1,3-benzoxazole (300 mg, 0.990 mmol, 1 equiv) and tert-butyl N-[1-(6-chloro-1,5-naphthyridin-2-yl) pyrrolidin-3-yl]-N-isopropylcarbamate (580.23 mg, 1.485 mmol, 1.5 equiv) in dioxane (16 mL) and H₂O (4 mL) were added Pd(dppf)Cl₂·CH₂Cl₂ (80.61 mg, 0.099 mmol, 0.1 equiv) and K3PO4 (630.14 mg, 2.970 mmol, 3 equiv) in portions at room temperature. A mixture was stirred for 2 hr at 100° C. under N2 atmosphere. The mixture was allowed to cool down to room temperature. The reaction was quenched with water at room temperature. The resulting mixture was extracted with ethyl acetate (3×20 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:1) to afford tert-butyl N-isopropyl-N-{1-[6-(5-methoxy-2,4-dimethyl-1,3-benzoxazol-6-yl)-1,5-naphthyridin-2-yl]pyrrolidin-3-yl}carbamate (310 mg, 58%) as a solid. LCMS (ES, m/z): 532 [M+H]

### Synthesis of Intermediate C88

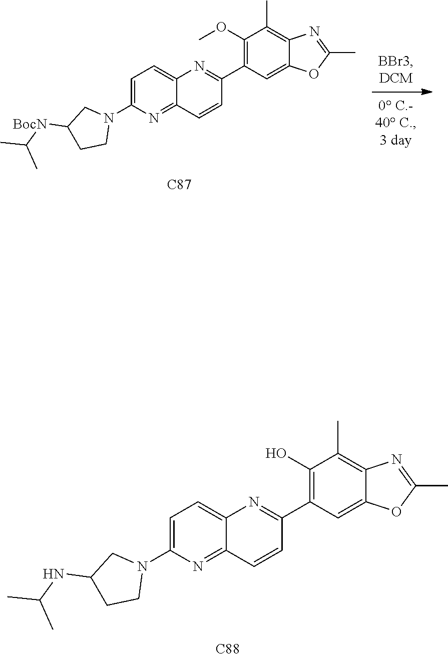

Into a 20 mL 4-necked round-bottom flask were added tert-butyl N-isopropyl-N-{1-[6-(5-methoxy-2,4-dimethyl-1,3-benzoxazol-6-yl)-1,5-naphthyridin-2-yl]pyrrolidin-3-yl}carbamate (280 mg, 0.527 mmol, 1 equiv) and DCM (6 mL) at room temperature. To the above mixture was added BBr3 (1319.38 mg, 5.270 mmol, 10 equiv) dropwise over 10 min at 0° C. The resulting mixture was stirred for additional 3 days at room temperature. The reaction was quenched by the addition of methanol (3 mL) at 0° C. The resulting mixture was extracted with ethyl acetate (1×20 mL). The combined organic layers were washed with water (3×10 mL), dried over anhydrous Na2SO4. After filtration, the filtrate was concentrated under reduced pressure. The crude product was purified by Prep-HPLC (Condition 1, Gradient 29) to afford 6-{6-[3-(isopropylamino)pyrrolidin-1-yl]-1,5-naphthyridin-2-yl}-2,4-dimethyl-1,3-benzoxazol-5-ol (45.2 mg, 20%) as a solid. LCMS (ES, m/z): 418 [M+H]

### Synthesis of Compounds 401 and 402

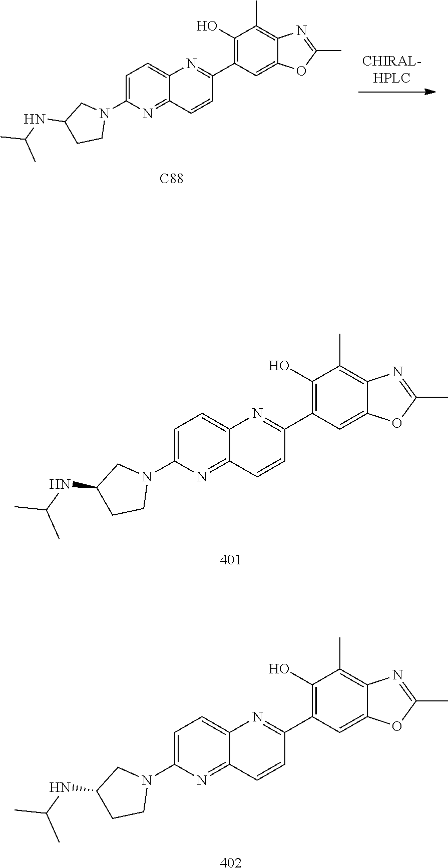

The 6-{6-[3-(isopropylamino)pyrrolidin-1-yl]-1,5-naphthyridin-2-yl}-2,4-dimethyl-1,3-benzoxazol-5-ol (110 mg, 0.263 mmol, 1 equiv) was purified by Chiral-Prep-HPLC (Condition 4, Gradient 4) to afford 6-{6-[(3S)-3-(isopropylamino)pyrrolidin-1-yl]-1,5-naphthyridin-2-yl}-2,4-dimethyl-1,3-benzoxazol-5-ol (36.4 mg, 33.09%) and 6-{6-[(3R)-3-(isopropylamino)pyrrolidin-1-yl]-1,5-naphthyridin-2-yl}-2,4-dimethyl-1,3-benzoxazol-5-ol (36.8 mg, 33%) as a solid.

### Analysis Data

| Compound Structure | Analysis data |
|---|---|
| 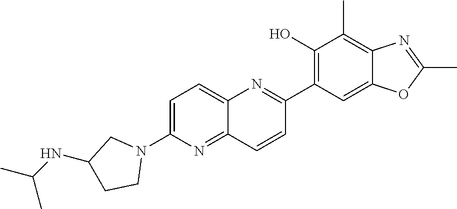 [SMILES: Cc1nc2c(C)c(O)c(-c3ccc4nc(N5CCC(NC(C)C)C5)ccc4n3)cc2o1] | LCMS- (ES, m/z): 418 [M + H] HNMR: ¹H NMR (400 MHz, DMSO-d₆) δ 14.99 (s, 1H), 8.41 (d, J = 9.3 Hz, 1H), 8.25 (s, 1H), 8.13 (d, J = 9.3 Hz, 1H), 8.07 (d, J = 9.1 Hz, 1H), 7.16 (d, J = 9.3 Hz, 1H), 3.78 (d, J = 8.3 Hz, 1H), 3.70 (s, 1H) 3.52 (s, 2H), 3.32 (s, 1H), 2.85 (p, J = 6.3 Hz, 1H), 2.61 (s, 3H), 2.40 (s, 3H), 2.15 (dt, J = 12.4, 6.3 Hz, 1H), 1.79 (dt, J = 14.1, 7.2 Hz, 1H), 1.72 (s, 1H), 1.02 (dd, J = 6.2, 4.6 Hz, 6H). |
| 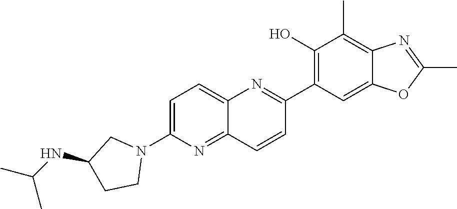 [SMILES: Cc1nc2c(C)c(O)c(-c3ccc4nc(N5CC[C@@H](NC(C)C)C5)ccc4n3)cc2o1] | LCMS- (ES, m/z): 418 [M + H] HNMR: ¹H NMR (400 MHz, DMSO-d₆) δ 14.99 (s, 1H), 8.42 (d, J = 9.3 Hz, 1H), 8.26 (s, 1H), 8.14 (d, J = 9.3 Hz, 1H), 8.08 (d, J = 9.1 Hz, 1H), 7.17 (d, J = 9.3 Hz, 1H), 3.79 (s, 1H), 3.71 (s, 1H), 3.53 (s, 2H), 3.33 (s, 1H), 2.90-2.82 (m, 1H), 2.61 (s, 3H), 2.40 (s, 3H), 2.16 (dd, J = 11.6, 5.9 Hz, 1H), 1.81 (s, 2H), 1.02 (dd, J = 6.2, 4.5 Hz, 6H). |
| 401 | |
| 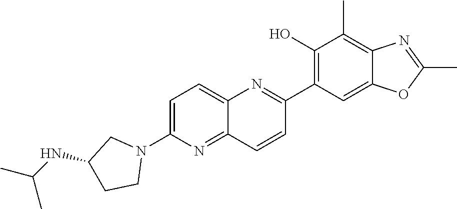 [SMILES: Cc1nc2c(C)c(O)c(-c3ccc4nc(N5CC[C@H](NC(C)C)C5)ccc4n3)cc2o1] | LCMS (ES, m/z): 418 [M + H] HNMR: ¹H NMR (400 MHz, DMSO-d₆) δ 14.99 (s, 1H), 8.41 (d, J = 9.2 Hz, 1H), 8.25 (s, 1H), 8.14 (d, J = 9.3 Hz, 1H), 8.08 (d, J = 9.1 Hz, 1H), 7.17 (d, J = 9.3 Hz, 1H), 3.79 (s, 1H), 3.70 (s, 1H), 3.53 (t, J = 9.2 Hz, 2H), 3.33 (s, 1H), 2.87 (t, J = 6.4 Hz, 1H), 2.61 (s, 3H), 2.40 (s, 3H), 2.16 (dd, J = 11.9, 6.3 Hz, 1H), 1.81 (s, 2H), 1.03 (dd, J = 6.2, 4.5 Hz, 6H). |
| 402 | |

### Example 138: Synthesis of Compound 346

### Synthesis of Intermediate C89

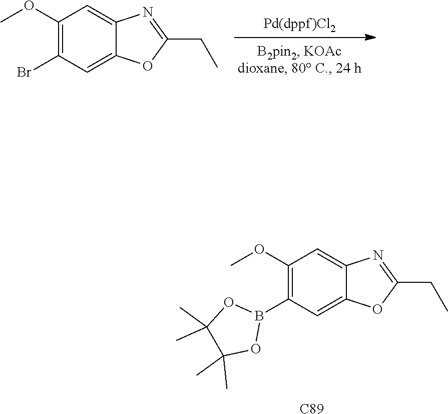 [SMILES: CCc1nc2cc(OC)c(Br)cc2o1 ; CCc1nc2cc(OC)c(B3OC(C)(C)C(C)(C)O3)cc2o1 ; "C89"]

Into a 40 mL round-bottom flask were added 6-bromo-2-ethyl-5-methoxy-1,3-benzoxazole (200 mg, 0.781 mmol, 1 equiv) and bis(pinacolato)diboron (397 mg, 1.562 mmol, 2 equiv) at room temperature. To the above mixture was added potassium acetate (230 mg, 2.343 mmol, 3 equiv) and Pd(dppf)Cl₂ (64 mg, 0.078 mmol, 0.1 equiv) in portions over 10 min at room temperature. The resulting mixture was stirred for additional 24 hr at 80° C. The mixture was allowed to cool down to room temperature. The resulting mixture was extracted with ethyl acetate (2×30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (5:1) to afford 2-ethyl-5-methoxy-6-(4,4,5,5-tetramethyl-1,3,2-dioxaborolan-2-yl)-1,3-benzoxazole (650 mg, 96%) as a solid. LCMS (ES, m/z): 304 [M+H]

### Synthesis of Intermediate C90

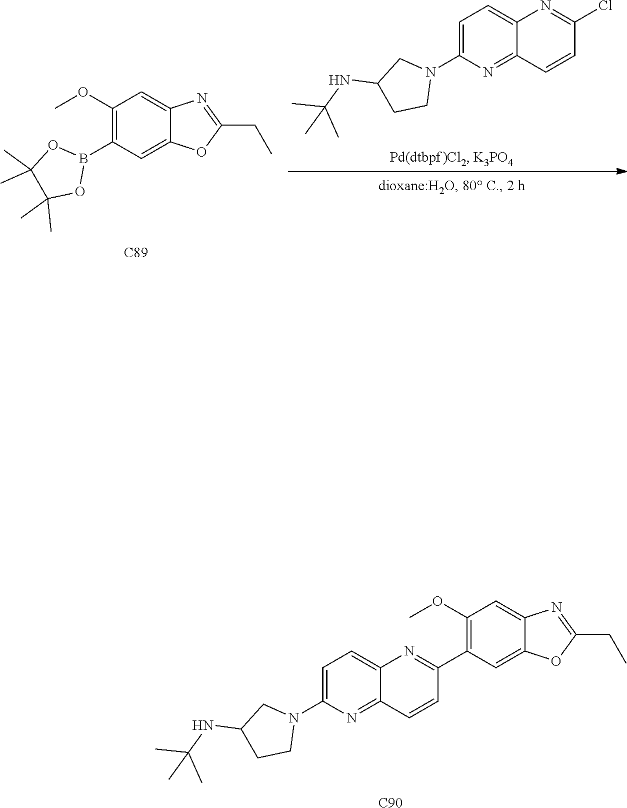

To a stirred solution of N-tert-butyl-1-(6-chloro-1,5-naphthyridin-2-yl)pyrrolidin-3-amine (620 mg, 2.034 mmol, 1 equiv) and Pd(DtBPF)Cl₂ (133 mg, 0.203 mmol, 0.1 equiv) in 1,4-dioxane (16 mL) and H₂O (4 mL) were added 2-ethyl-5-methoxy-6-(4,4,5,5-tetramethyl-1,3,2-dioxaborolan-2-yl)-1,3-benzoxazole (925 mg, 3.051 mmol, 1.5 equiv) and K₃PO₄ (1.29 g, 6.102 mmol, 3 equiv) dropwise at room temperature under N₂ atmosphere. The mixture was stirred for 2 h at 80° C. under N₂ atmosphere. The mixture was allowed to cool down to room temperature. The resulting mixture was extracted with EA (2×30 mL). The combined organic layers were washed with water (3×20 mL), dried over anhydrous Na₂SO₄. After filtration, the filtrate was concentrated under reduced pressure. The residue was purified by silica gel column chromatography, eluted with PE/EA (1:3) to afford N-tert-butyl-1-[6-(2-ethyl-5-methoxy-1,3-benzoxazol-6-yl)-1,5-naphthyridin-2-yl]pyrrolidin-3-amine (25 mg, 2%) as an oil. LCMS (ES, m/z): 446 [M+H]

### Synthesis of Compound 346

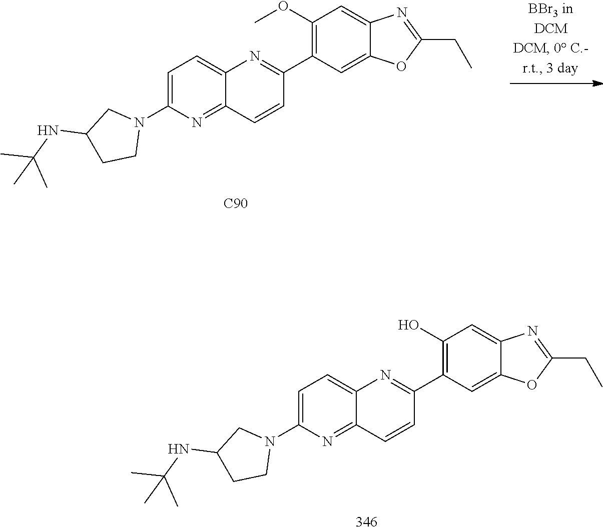

To a stirred solution of N-tert-butyl-1-[6-(2-ethyl-5-methoxy-1,3-benzoxazol-6-yl)-1,5-naphthyridin-2-yl]pyrrolidin-3-amine (20 mg, 0.045 mmol, 1 equiv) in DCM (5 mL) was added BBr₃ (225 mg, 0.900 mmol, 20 equiv) dropwise at 0° C. The mixture was stirred for 3 days at room temperature. The mixture was basified to pH 8 with saturated NaHCO₃. The resulting mixture was extracted with CH₂Cl₂ (3×30 mL). The combined organic layers were washed with water (3×20 mL), dried over anhydrous Na₂SO₄. After filtration, the filtrate was concentrated under reduced pressure. The crude product was purified by Prep-HPLC (Condition 3, Gradient 24) to afford 6-{6-[3-(tert-butylamino)pyrrolidin-1-yl]-1,5-naphthyridin-2-yl}-2-ethyl-1,3-benzoxazol-5-ol (7 mg, 36%) as a solid. LCMS (ES, m/z): 432 [M+H]¹H NMR (400 MHz, DMSO-d₆) δ 14.53 (s, 1H), 8.45-8.38 (m, 2H), 8.15-8.05 (m, 2H), 7.15 (d, J=11.6 Hz, 2H), 3.91-3.80 (m, 1H), 3.79-3.72 (m, 1H), 3.49 (dd, J=18.8, 9.9 Hz, 2H), 3.21-3.05 (m, 1H), 2.96 (q, J=7.5 Hz, 2H), 2.24-2.08 (m, 1H), 1.84-1.69 (s, 2H), 1.35 (t, J=7.5 Hz, 3H), 1.10 (s, 9H).

### Example 139: Synthesis of Compounds 334 and 336

### Synthesis of Intermediate C91

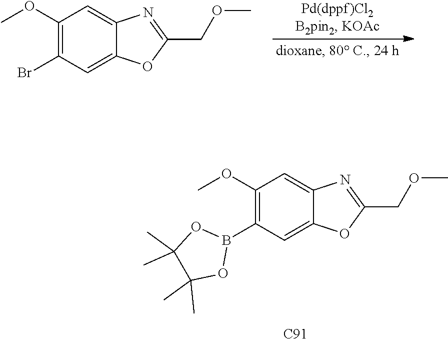

To a stirred solution of 6-bromo-5-methoxy-2-(methoxymethyl)-1,3-benzoxazole (250 mg, 0.919 mmol, 1 equiv) and Pd(dppf)Cl₂ (75 mg, 0.092 mmol, 0.1 equiv) in dioxane (20 mg) were added bis(pinacolato)diboron (467 mg, 1.838 mmol, 2 equiv) and potassium acetate (271 mg, 2.757 mmol, 3 equiv) in portions at room temperature. The mixture was stirred for 24 hr at 80° C. under N₂ atmosphere. The mixture was allowed to cool down to room temperature. The resulting mixture was extracted with ethyl acetate (3×30 mL). The combined organic layers were washed with water (2×30 mL), dried over anhydrous Na₂SO₄. After filtration, the filtrate was concentrated under reduced pressure. This resulted in 5-methoxy-2-(methoxymethyl)-6-(4,4,5,5-tetramethyl-1,3,2-dioxaborolan-2-yl)-1,3-benzoxazole (630 mg, 75%) as a solid. LCMS (ES, m/z): 320 [M+H]

### Synthesis of Intermediate C92

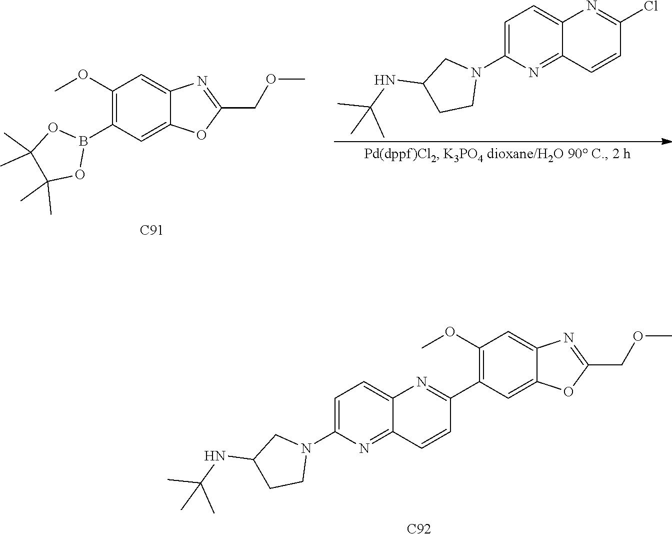

To a stirred solution of 5-methoxy-2-(methoxymethyl)-6-(4,4,5,5-tetramethyl-1,3,2-dioxaborolan-2-yl)-1,3-benzoxazole (630 mg, 1.974 mmol, 1 equiv) and Pd(dppf)Cl₂ (161 mg, 0.197 mmol, 0.1 equiv) in 1,4-dioxane (16 mL) and H₂O (4 mL) were added N-tert-butyl-1-(6-chloro-1,5-naphthyridin-2-yl) pyrrolidin-3-amine (902 mg, 2.961 mmol, 1.5 equiv) and K₃PO₄ (1.26 g, 5.922 mmol, 3 equiv) dropwise at room temperature. The mixture was stirred for 2 h at 90° C. under N₂ atmosphere. The mixture was allowed to cool down to room temperature. The resulting mixture was extracted with ethyl acetate (3×30 mL). The combined organic layers were washed with water (2×30 mL), dried over anhydrous Na₂SO₄. After filtration, the filtrate was concentrated under reduced pressure. The residue was purified by silica gel column chromatography, eluted with PE/EA (3:1) to afford N-tert-butyl-1-{6-[5-methoxy-2-(methoxymethyl)-1,3-benzoxazol-6-yl]-1,5-naphthyridin-2-yl}pyrrolidin-3-amine (320 mg, 35%) as a solid. LCMS (ES, m/z): 462 [M+H]

### Synthesis of Compounds 334 and 336

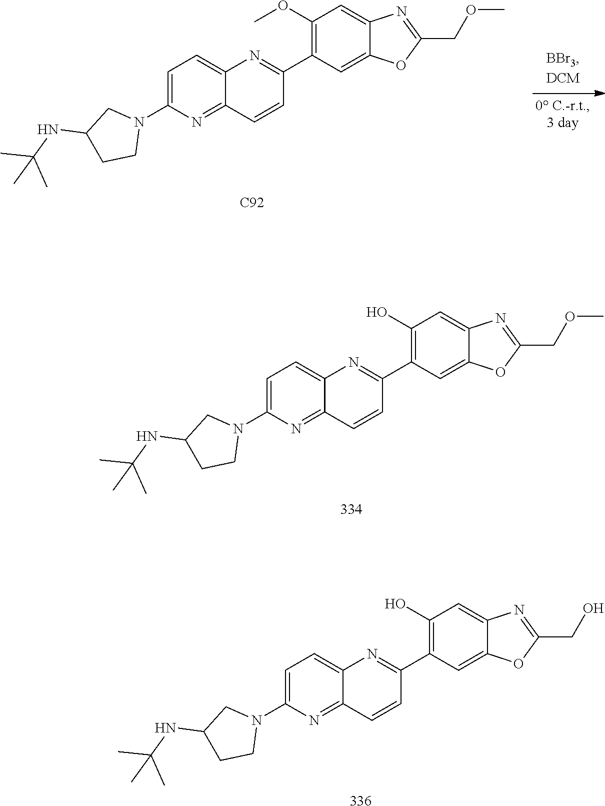

To a stirred solution of N-tert-butyl-1-{6-[5-methoxy-2-(methoxymethyl)-1,3-benzoxazol-6-yl]-1,5-naphthyridin-2-yl}pyrrolidin-3-amine (290 mg, 0.628 mmol, 1 equiv) in DCM (20 mL) were added BBr₃ (1.57 g, 6.280 mmol, 10 equiv) dropwise at 0° C. The mixture was stirred for 3 days at room temperature. The crude product was purified by Prep-HPLC (Condition 1, Gradient 18) to afford 6-{6-[3-(tert-butylamino) pyrrolidin-1-yl]-1,5-naphthyridin-2-yl}-2-(methoxymethyl)-1,3-benzoxazol-5-ol (9.6 mg, 30%) and 6-{6-[3-(tert-butylamino)pyrrolidin-1-yl]-1,5-naphthyridin-2-yl}-2-(hydroxymethyl)-1,3-benzoxazol-5-ol (14.8 mg, 50%) as a solid. LCMS (ES, m/z): 448 [M+H] LCMS (ES, m/z): 434 [M+H]

| Compound Structure | Analysis data |
|---|---|
| 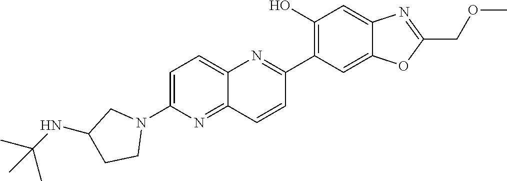 [SMILES: COCc1nc2cc(O)c(-c3ccc4nc(N5CCC(NC(C)(C)C)C5)ccc4n3)cc2o1] | LCMS- (ES, m/z): 448 [M + H] HNMR: ¹H NMR (400 MHz, DMSO- d₆) δ 8.07 (d, J = 9.2 Hz, 1H), 7.99 (d, J = 20.1 Hz, 2H), 7.91 (d, J = 8.8 Hz, 1H), 7.48 (s, 1H), 7.11 (d, J = 9.3 Hz, 1H), 5.89 (t, J = 6.3 Hz, 1H), 4.71 (d, J = 6.3 Hz, 2H), 3.90 (s, 4H), 3.81- 3.70 (m, 1H), 3.60-3.52 (m, 2H), 3.19-3.07 (m, 1H), 2.19-2.05 (m, 1H), 1.89-1.60 (m, 2H), 1.10 (s, 9H). |
| 334 | |
| 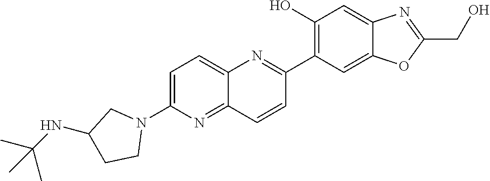 [SMILES: CC(C)(C)NC1CCN(c2ccc3nc(-c4cc5oc(CO)nc5cc4O)ccc3n2)C1] | LCMS (ES, m/z): 434 [M + H] HNMR: ¹H NMR (400 MHz, DMSO- d₆) δ 14.55 (s, 1H), 8.50-8.41 (m, 2H), 8.16-8.06 (m, 2H), 7.21-7.13 (m, 2H), 5.88 (t, J = 6.3 Hz, 1H), 4.70 (d, J = 6.2 Hz, 2H), 3.98-3.87 (m, 1H), 3.85-3.72 (m, 1H), 3.68-3.53 (m, 2H), 3.18-3.14 (m, 1H), 2.23- 2.19 (m, 1H), 1.89-1.76 (m, 2H), 1.10 (s, 9H). |
| 336 | |

### Example 140: Synthesis of Compounds 378 and 379

### Synthesis of Intermediates C93 and C94

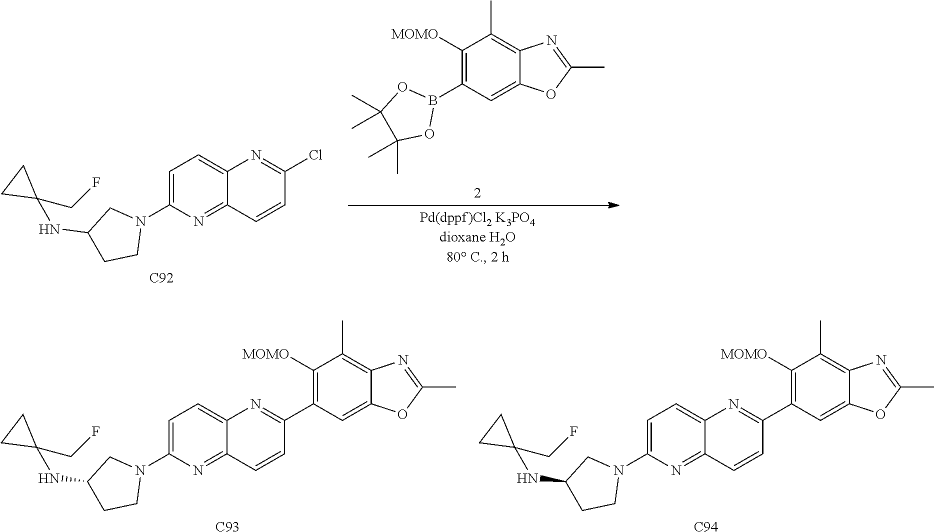

A solution of 1-(6-chloro-1,5-naphthyridin-2-yl)-N-[1-(fluoromethyl)cyclopropyl]pyrrolidin-3-amine (300 mg, 0.935 mmol, 1 equiv) and 5-(methoxymethoxy)-2,4-dimethyl-6-(4,4,5,5-tetramethyl-1,3,2-dioxaborolan-2-yl)-1,3-benzoxazole (373.90 mg, 1.122 mmol, 1.2 equiv) in dioxane (6 ml)/H₂O (1.2 mL) was treated with Pd(dppf)Cl₂ (76.5 mg, 0.0935 mmol, 0.1 equiv) under nitrogen atmosphere followed by the addition of K₃PO₄ (595.51 mg, 2.805 mmol, 3 equiv) in portions at room temperature. The mixture was stirred for 2 hr at 80° C. The mixture was allowed to cool down to room temperature. The resulting mixture was concentrated under vacuum. The residue was purified by silica gel column chromatography, eluted with PE/EA (1:1) to afford 156 mg crude. The crude product (156 mg) was purified by Prep-CHIRAL-HPLC with the following conditions (Condition 5, Gradient 1) to afford (3S)—N-[1-(fluoromethyl)cyclopropyl]-1-{6-[5-(methoxymethoxy)-2,4-dimethyl-1,3-benzoxazol-6-yl]-1,5-naphthyridin-2-yl}pyrrolidin-3-amine (55 mg, 11%) as a solid and (3R)—N-[1-(fluoromethyl)cyclopropyl]-1-{6-[5-(methoxymethoxy)-2,4-dimethyl-1,3-benzoxazol-6-yl]-1,5-naphthyridin-2-yl}pyrrolidin-3-amine (50 mg, 10%) as a solid.

### Synthesis of Compound 378

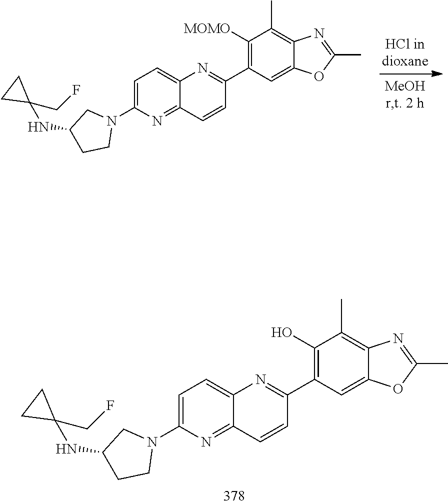

A mixture of (3S)—N-[1-(fluoromethyl)cyclopropyl]-1-{6-[5-(methoxymethoxy)-2,4-dimethyl-1,3-benzoxazol-6-yl]-1,5-naphthyridin-2-yl}pyrrolidin-3-amine (55 mg, 0.112 mmol, 1 equiv) and HCl(gas) in 1,4-dioxane (1 mL) in methanol (1 mL) was stirred for 2 hr at room temperature under nitrogen atmosphere. The resulting mixture was concentrated under vacuum. The crude product (55 mg) was purified by Prep-HPLC (Condition 1, Gradient 30) to afford 6-{6-[(3S)-3-{[1-(fluoromethyl)cyclopropyl]amino}pyrrolidin-1-yl]-1,5-naphthyridin-2-yl}-2,4-dimethyl-1,3-benzoxazol-5-ol (23 mg, 45%) as a solid. LCMS (ES, m/z): 448 [M+H]+H NMR (400 MHz, DMSO-d₆) δ 14.98 (s, 1H), 8.41 (d, J=9.3 Hz, 1H), 8.25 (s, 1H), 8.14 (dd, J=9.3, 0.8 Hz, 1H), 8.08 (dd, J=9.1, 0.8 Hz, 1H), 7.15 (d, J=9.3 Hz, 1H), 4.47 (s, 1H), 4.34 (s, 1H), 3.78 (s, 1H), 3.79-3.67 (m, 2H), 3.55 (t, J=8.3 Hz, 1H), 2.75-2.64 (m, 1H), 2.61 (s, 3H), 2.40 (s, 3H), 2.14 (dt, J=12.5, 6.3 Hz, 1H), 1.89 (dd, J=12.4, 6.6 Hz, 1H), 0.62 (d, J=3.1 Hz, 4H).

### Synthesis of Compound 379

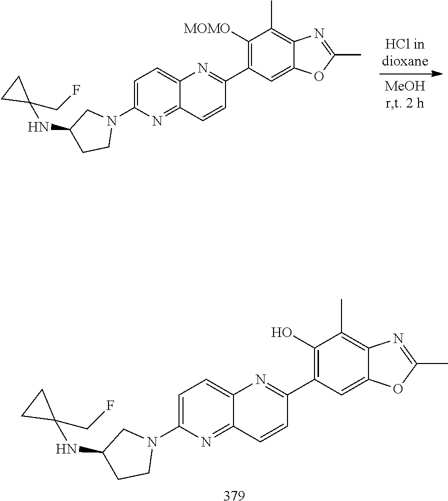

A mixture of (3R)-N-[1-(fluoromethyl)cyclopropyl]-1-{6-[5-(methoxymethoxy)-2,4-dimethyl-1,3-benzoxazol-6-yl]-1,5-naphthyridin-2-yl}pyrrolidin-3-amine (50 mg, 0.102 mmol, 1 equiv) and HCl(gas) in 1,4-dioxane (1 mL) in MeOH (1 mL) was stirred for 2 h at room temperature under nitrogen atmosphere. The resulting mixture was concentrated under vacuum. The crude product (50 mg) was purified by Prep-HPLC (Condition 1, Gradient 30) to afford 6-{6-[(3R)-3-{[1-(fluoromethyl)cyclopropyl]amino}pyrrolidin-1-yl]-1,5-naphthyridin-2-yl}-2,4-dimethyl-1,3-benzoxazol-5-ol (14.1 mg, 30%) as a solid. LCMS (ES, m/z): 448 [M+H]+ ¹H NMR (400 MHz, DMSO-d₆) δ 14.98 (s, 1H), 8.41 (d, J=9.3 Hz, 1H), 8.25 (s, 1H), 8.13 (dd, J=9.3, 0.8 Hz, 1H), 8.08 (d, J=9.1 Hz, 1H), 7.15 (d, J=9.3 Hz, 1H), 4.47 (s, 1H), 4.34 (s, 1H), 3.77 (d, J=9.5 Hz, 1H), 3.70 (s, 2H), 3.65 (s, 1H), 3.54 (d, J=9.4 Hz, 1H), 2.70 (s, 1H), 2.61 (s, 3H), 2.40 (s, 3H), 2.14 (dq, J=12.6, 6.3 Hz, 1H), 1.89 (dd, J=12.5, 6.5 Hz, 1H), 0.62 (d, J=3.7 Hz, 4H).

### Example 141: Synthesis of Compounds 391 and 392

### Synthesis of Intermediate C95

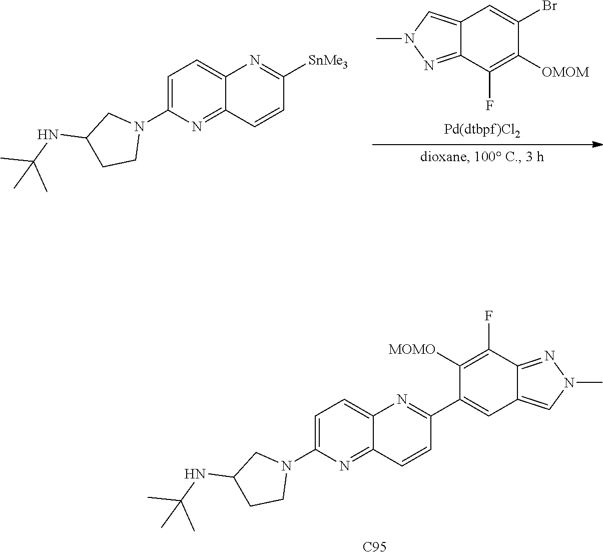

To a solution of N-tert-butyl-1-[6-(trimethylstannyl)-1,5-naphthyridin-2-yl]pyrrolidin-3-amine (1.5 g, 3.463 mmol, 1 equiv) and 5-bromo-7-fluoro-6-(methoxymethoxy)-2-methylindazole (1.00 g, 3.463 mmol, 1 equiv) in 1,4-dioxane (20 mL) were added Pd(DtBPF)Cl₂ (0.23 g, 0.346 mmol, 0.1 equiv). After stirring for 3 h at 100° C. under a nitrogen atmosphere, the resulting mixture was concentrated under reduced pressure. The residue was purified by silica gel column chromatography, eluted with DCM/MeOH (10:1) to afford N-tert-butyl-1-{6-[7-fluoro-6-(methoxymethoxy)-2-methylindazol-5-yl]-1,5-naphthyridin-2-yl}pyrrolidin-3-amine (500 mg, 30%) as a solid.

### Synthesis of Compounds 391 and 392

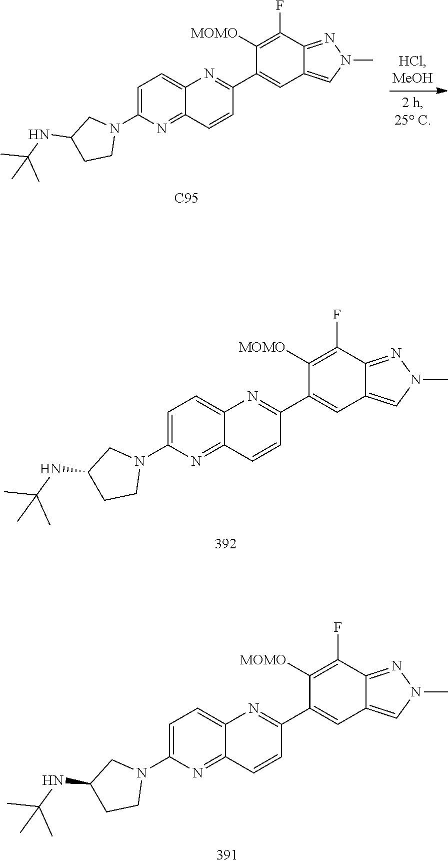

To a solution of N-tert-butyl-1-{6-[7-fluoro-6-(methoxymethoxy)-2-methylindazol-5-yl]-1,5-naphthyridin-2-yl}pyrrolidin-3-amine (500 mg, 1.045 mmol, 1 equiv) in MeOH (5 mL) were added HCl(gas) in 1,4-dioxane (5 mL). After stirring for 2 h at 25° C. under a nitrogen atmosphere, the resulting mixture was concentrated under reduced pressure. The crude product was purified by Chiral-Prep-HPLC (Condition 4, Gradient 4) to afford the crude product. The crude product was purified by Chiral-Prep-HPLC (Condition 3, Gradient 21) to afford 5-{6-[(3S)-3-(tert-butylamino)pyrrolidin-1-yl]-1,5-naphthyridin-2-yl}-7-fluoro-2-methylindazol-6-ol (27.7 mg, 6.1%) as a solid, 5-{6-[(3R)-3-(tert-butylamino)pyrrolidin-1-yl]-1,5-naphthyridin-2-yl}-7-fluoro-2-methylindazol-6-ol (67.7 mg, 15%) as a solid.

| Compound Structure | Analysis data |
|---|---|
| 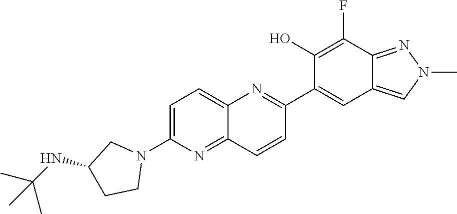 [SMILES: Cn1cc2cc(-c3ccc4nc(N5CC[C@H](NC(C)(C)C)C5)ccc4n3)c(O)c(F)c2n1] | LCMS: (ES, m/z): 435 [M + H] + ¹H NMR (400 MHz, DMSO-d₆) δ 14.45 (s, 1H), 8.48 (d, J = 2.6 Hz, [symbol] H), 8.40 (t, J = 4.7 Hz, 2H), 8.13 (d, J = 9.3 Hz, 1H), 8.08 (d, J = 9.1 Hz, 1H), 7.15 (d, J = 9.3 Hz, 1H), 4.16 (s, 3H), 3.86 (s, 1H), 3.71 (s, 1H), 3.49 (ddd, J = 18.0, 12.0, 7.3 Hz, 2H), 3.12 (t, J = 8.9 Hz, 1H), 2.18 (q, J = 4.7, 4.0 Hz, 1H), 1.77 (d, J = 11.0 Hz, 2H), 1.09 (s, 9H). |
| 392 | |
| 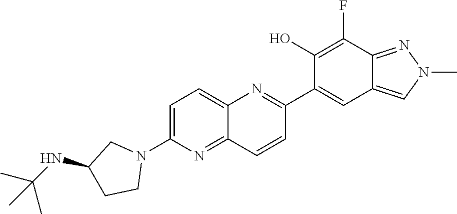 [SMILES: Cn1cc2cc(-c3ccc4nc(N5CC[C@@H](NC(C)(C)C)C5)ccc4n3)c(O)c(F)c2n1] | LCMS: (ES, m/z): 435 [M + H] ⁺ ¹H NMR: (400 MHz, DMSO-d₆) δ 14.45 (s, 1H), 8.48 (d, J = 2.6 Hz, [symbol] H), 8.40 (t, J = 4.7 Hz, 2H), 8.13 (d, J = 9.3 Hz, 1H), 8.08 (d, J = 9.1 Hz, 1H), 7.15 (d, J = 9.3 Hz, 1H), 4.16 (s, 3H), 3.86 (s, 1H), 3.71 (s, 1H), 3.49 (ddd, J = 18.0, 12.0, 7.3 Hz, 2H), 3.12 (t, J = 8.9 Hz, 1H), 2.18 (q, J = 4.7, 4.0 Hz, 1H), 1.77 (d, J = 11.0 Hz, 2H), 1.09 (s, 9H). |
| 391 | |

[symbol]

indicates data missing or illegible when filed

### Example 142: Synthesis of Compounds 372 and 373

### Synthesis of Intermediates C96 and C97

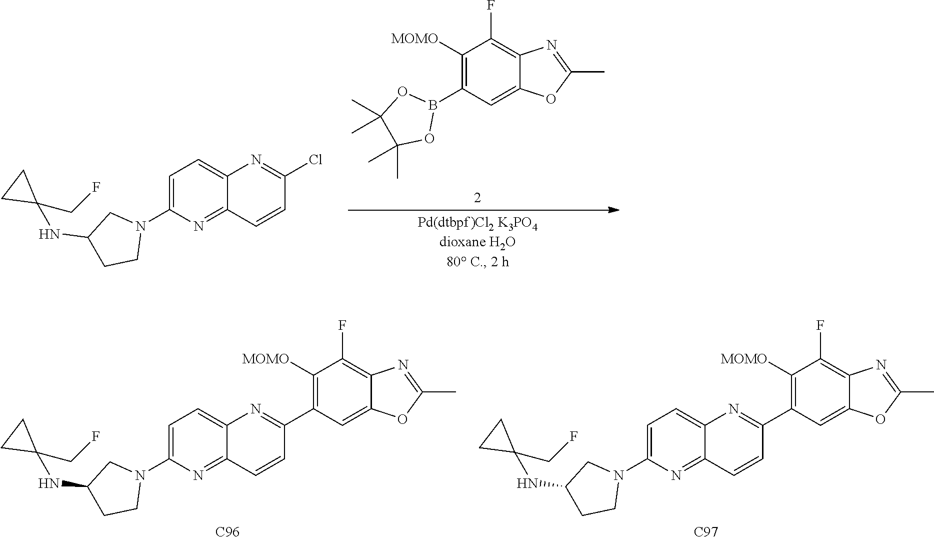

A solution of 1-(6-chloro-1,5-naphthyridin-2-yl)-N-[1-(fluoromethyl)cyclopropyl]pyrrolidin-3-amine (300 mg, 0.935 mmol, 1 equiv) and 4-fluoro-5-(methoxymethoxy)-2-methyl-6-(4,4,5,5-tetramethyl-1,3,2-dioxaborolan-2-yl)-1,3-benzoxazole (378.35 mg, 1.122 mmol, 1.2 equiv) in dioxane (1.2 mL) and H₂O (6 mL) was treated with Pd(dtbpf)Cl₂ (60.95 mg, 0.094 mmol, 0.1 equiv) at room temperature under nitrogen atmosphere followed by the addition of K₃PO₄ (390.56 mg, 2.805 mmol, 3 equiv) in portions at room temperature. The mixture was stirred for 2 hr at 80° C. The mixture was allowed to cool down to room temperature. The resulting mixture was concentrated under vacuum. The residue was purified by silica gel column chromatography, eluted with CH₂Cl₂/MeOH (10:1) to afford 1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (200 mg) as a brown solid. The crude product (200 mg) was purified by Prep-CHIRAL-HPLC (Condition 4, Gradient 1) to afford (3R)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (60 mg, 12%) as a solid and (3S)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (55 mg, 11%) as a solid.

### Synthesis of Compound 372

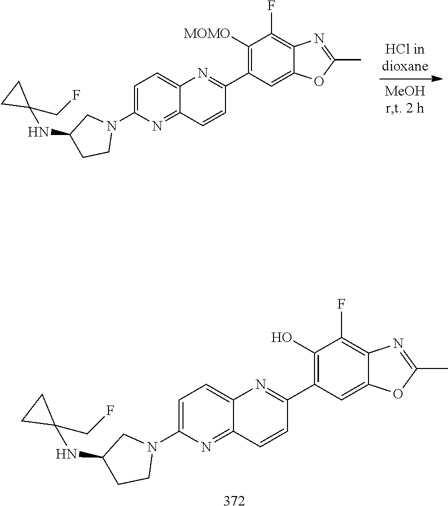

A mixture of (3R)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (60 mg, 0.121 mmol, 1 equiv) and HCl(gas) in 1,4-dioxane (1 mL) in MeOH (1 mL) was stirred for 2 h at room temperature under nitrogen atmosphere. The resulting mixture was concentrated under vacuum. The crude product (55 mg) was purified by Prep-HPLC (Condition 1, Gradient 31) to afford 4-fluoro-6-{6-[(3R)-3-{[1-(fluoromethyl)cyclopropyl]amino}pyrrolidin-1-yl]-1,5-naphthyridin-2-yl}-2-methyl-1,3-benzoxazol-5-ol (17.6 mg, 32%) as a solid. LCMS (ES, m/z): 452 [M+H]+ ¹H NMR (400 MHz, DMSO-d₆) δ 15.06 (s, 1H), 8.44 (d, J=9.2 Hz, 1H), 8.32 (d, J=1.3 Hz, 1H), 8.13 (ddd, J=19.0, 9.2, 0.8 Hz, 2H), 7.17 (d, J=9.3 Hz, 1H), 4.47 (s, 1H), 4.34 (s, 1H), 3.77 (d, J=9.2 Hz, 1H), 3.70 (s, 2H), 3.54 (d, J=9.4 Hz, 1H), 3.34 (s, 1H), 2.71 (d, J=4.1 Hz, 1H), 2.64 (s, 3H), 2.14 (dq, J=12.6, 6.3 Hz, 1H), 1.94-1.86 (m, 1H), 0.62 (d, J=4.6 Hz, 4H).

### Synthesis of Compound 373

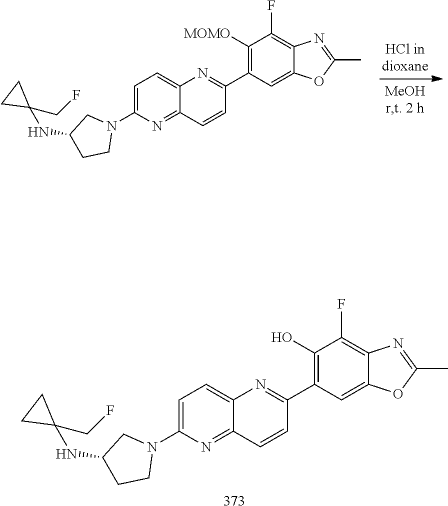

A mixture of (3S)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (55 mg, 0.111 mmol, 1 equiv) and HCl(gas) in 1,4-dioxane (1 mL) in MeOH (1 mL) was stirred for 2 h at room temperature under nitrogen atmosphere. The resulting mixture was concentrated under vacuum. The crude product (50 mg) was purified by Prep-HPLC (Condition 1, Gradient 32) to afford 4-fluoro-6-{6-[(3S)-3-{[1-(fluoromethyl)cyclopropyl]amino}pyrrolidin-1-yl]-1,5-naphthyridin-2-yl}-2-methyl-1,3-benzoxazol-5-ol (9.6 mg, 19%) as a solid. LCMS (ES, m/z): 452 [M+H]+ ¹H NMR (400 MHz, DMSO-d₆) δ 15.06 (s, 1H), 8.44 (d, J=9.2 Hz, 1H), 8.32 (d, J=1.4 Hz, 1H), 8.13 (dd, J=19.1, 9.2 Hz, 2H), 7.17 (d, J=9.3 Hz, 1H), 4.47 (s, 1H), 4.34 (s, 1H), 3.78 (s, 1H), 3.70 (s, 2H), 3.54 (d, J=9.3 Hz, 1H), 3.31 (s, 1H), 2.71 (d, J=4.2 Hz, 1H), 2.64 (s, 3H), 2.14 (dt, J=12.4, 6.3 Hz, 1H), 1.90 (s, 1H), 0.62 (d, J=4.5 Hz, 4H).

### Example 143: Synthesis of Compounds 368 and 369

### Synthesis of Intermediate C98

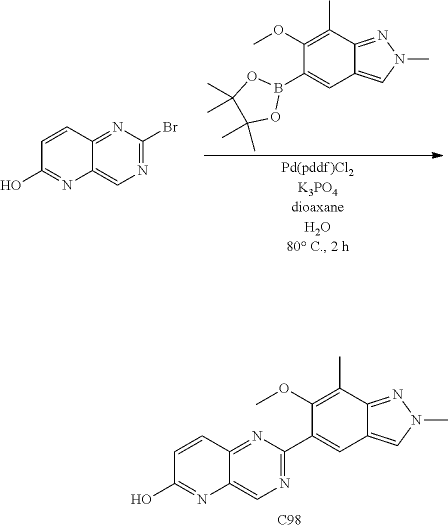

A solution of 2-bromopyrido[3,2-d]pyrimidin-6-ol (450 mg, 1.991 mmol, 1 equiv) in dioxane (1.4 mL) and H₂O (9 mL) was treated with Pd(dppf)Cl₂ (162.18 mg, 0.199 mmol, 0.1 equiv) at room temperature under nitrogen atmosphere followed by the addition of K₃PO₄ (1267.77 mg, 5.973 mmol, 3 equiv) in portions at room temperature. The mixture was stirred for 2 hr at 80° C. The mixture was allowed to cool down to room temperature. The resulting mixture was concentrated under vacuum. The residue was purified by silica gel column chromatography, eluted with CH₂Cl₂/MeOH (10:1) to afford 2-(6-methoxy-2,7-dimethylindazol-5-yl) pyrido[3,2-d]pyrimidin-6-ol (400 mg, 62%) as a solid.

### Synthesis of Intermediate C99 and C100

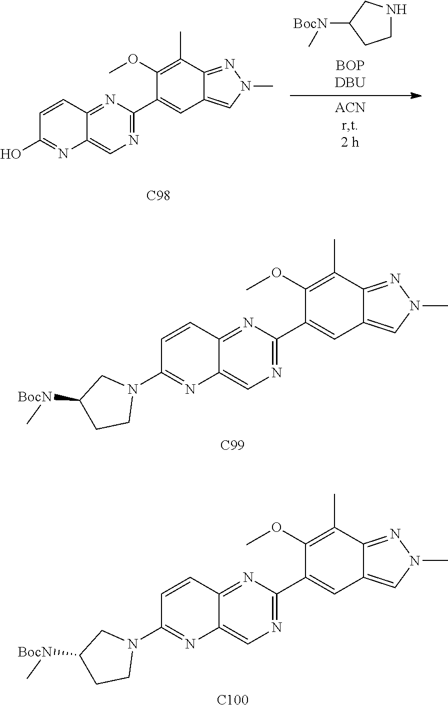

A solution of 2-(6-methoxy-2,7-dimethylindazol-5-yl)pyrido[3,2-d]pyrimidin-6-ol (390 mg, 1.214 mmol, 1 equiv) in acetonitrile (252.62 mL) was treated with BOP (1073.58 mg, 2.428 mmol, 2 equiv) at room temperature under nitrogen atmosphere followed by the addition of DBU (554.31 mg, 3.642 mmol, 3 equiv) dropwise at room temperature. The mixture was stirred for 2 hr at room temperature. The resulting mixture was concentrated under vacuum. The residue was purified by reverse flash chromatography (Condition 7, Gradient 3) to afford 180 mg product as a solid. The crude product was purified by Prep-chiral-HPLC (Condition 4, Gradient 5) to afford tert-butyl N-[(3R)-1-[2-(6-methoxy-2,7-dimethylindazol-5-yl)pyrido[3,2-d]pyrimidin-6-yl]pyrrolidin-3-yl]-N-methylcarbamate (70 mg, 11%) as a solid and tert-butyl N-[(3S)-1-[2-(6-methoxy-2,7-dimethylindazol-5-yl)pyrido[3,2-d]pyrimidin-6-yl]pyrrolidin-3-yl]-N-methylcarbamate (80 mg, 13%) as a solid.

### Synthesis of Compound 369

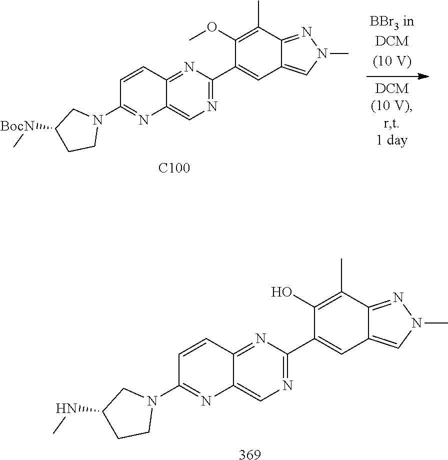

A mixture of tert-butyl N-[(3 S)-1-[2-(6-methoxy-2,7-dimethylindazol-5-yl)pyrido[3,2-d]pyrimidin-6-yl]pyrrolidin-3-yl]-N-methylcarbamate (70 mg, 0.139 mmol, 1 equiv) and BBr3 (0.5 mL, 5.289 mmol, 38.05 equiv) in dichloromethane (2 mL, 31.461 mmol, 226.34 equiv) was stirred for 1 day at room temperature under nitrogen atmosphere. The reaction was quenched with methanol at 0° C. The resulting mixture was concentrated under vacuum. The crude product was purified by Prep-HPLC (Condition 1, Gradient 2) to afford 2,7-dimethyl-5-{6-[(3S)-3-(methylamino) pyrrolidin-1-yl]pyrido[3,2-d]pyrimidin-2-yl}indazol-6-ol (30.6 mg, 56%) as a solid. LCMS (ES, m/z): 390 [M+H]+ ¹H NMR (400 MHz, DMSO-d₆) δ 13.31 (s, 1H), 9.25 (d, J=0.8 Hz, 1H), 8.84 (d, J=0.8 Hz, 1H), 8.38 (s, 1H), 8.16 (dd, J=9.4, 0.8 Hz, 1H), 7.41 (d, J=9.4 Hz, 1H), 4.13 (s, 3H), 3.72 (dd, J=11.1, 5.8 Hz, 1H), 3.67 (s, 2H), 3.62 (s, 1H), 3.43 (s, 1H), 2.39 (d, J=0.7 Hz, 3H), 2.33 (s, 3H), 2.11 (dt, J=12.9, 6.4 Hz, 2H), 1.89 (s, 1H).

### Synthesis of Compound 368

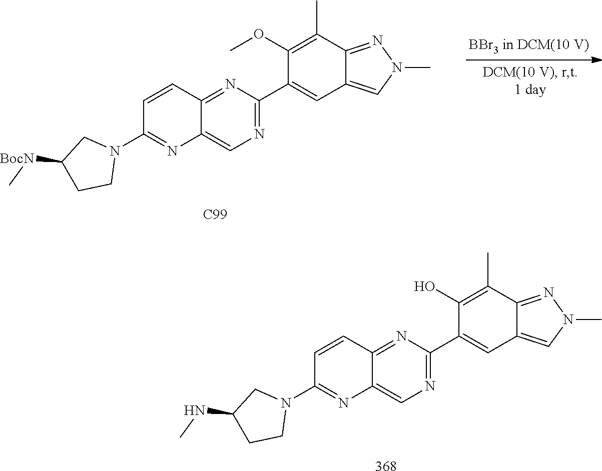

A solution of tert-butyl N-[(3R)-1-[2-(6-methoxy-2,7-dimethylindazol-5-yl)pyrido[3,2-d]pyrimidin-6-yl]pyrrolidin-3-yl]-N-methylcarbamate (80 mg, 0.159 mmol, 1 equiv) and BBr₃ (0.5 mL, 5.289 mmol, 33.29 equiv) in DCM (2 mL) was stirred for 1 days at room temperature under nitrogen atmosphere. The reaction was quenched with MeOH at 0° C. The resulting mixture was concentrated under vacuum. The crude product was purified by Prep-HPLC (Condition 1, Gradient 2) to afford 2,7-dimethyl-5-{6-[(3R)-3-(methylamino)pyrrolidin-1-yl]pyrido[3,2-d]pyrimidin-2-yl}indazol-6-ol (29.6 mg, 47%) as a solid. LCMS (ES, m/z): 390 [M+H]+ ¹H NMR (400 MHz, DMSO-d₆) δ 13.31 (s, 1H), 9.25 (d, J=0.8 Hz, 1H), 8.84 (d, J=0.8 Hz, 1H), 8.38 (s, 1H), 8.16 (dd, J=9.4, 0.8 Hz, 1H), 7.41 (d, J=9.4 Hz, 1H), 4.13 (s, 3H), 3.72 (dd, J=11.1, 5.8 Hz, 1H), 3.67 (s, 2H), 3.62 (s, 1H), 3.43 (s, 1H), 2.39 (d, J=0.7 Hz, 3H), 2.33 (s, 3H), 2.11 (dt, J=12.9, 6.4 Hz, 1H), 1.89 (s, 2H).

### Example 144: Synthesis of Compounds 380 and 381

### Synthesis of Intermediate C101

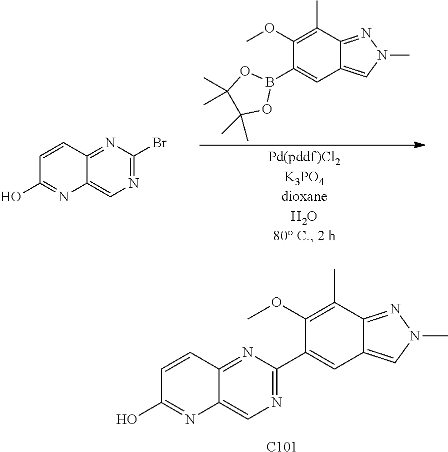

A mixture of 2-bromopyrido[3,2-d]pyrimidin-6-ol (300 mg, 1.327 mmol, 1 equiv) and 7-fluoro-6-methoxy-2-methyl-5-(4,4,5,5-tetramethyl-1,3,2-dioxaborolan-2-yl) indazole (487.59 mg, 1.592 mmol, 1.2 equiv) and Pd(dppf)Cl₂ (97.12 mg, 0.133 mmol, 0.1 equiv) and K₃PO₄(845.18 mg, 3.981 mmol, 3 equiv) in dioxane (6 mL) and H₂O (1.2 mL) was stirred for 2 hr at 80° C. under nitrogen atmosphere. The mixture was allowed to cool down to room temperature. The resulting mixture was concentrated under vacuum. The residue was purified by silica gel column chromatography, eluted with CH₂Cl₂/MeOH (10:1) to afford 2-(7-fluoro-6-methoxy-2-methylindazol-5-yl)pyrido[3,2-d]pyrimidin-6-ol (150 mg, 34%) as a solid.

### Synthesis of Intermediate C102

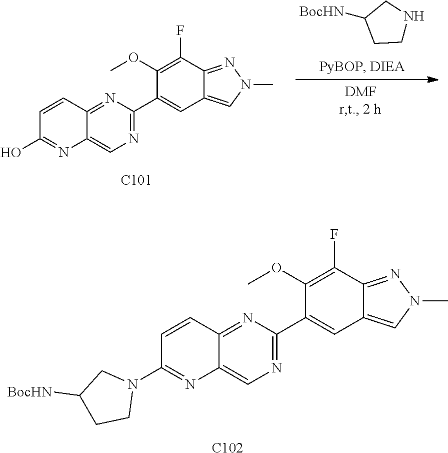 [SMILES: COc1c(-c2ncc3nc(O)ccc3n2)cc2cn(C)nc2c1F ; CC(C)(C)OC(=O)NC1CCNC1 ; "C101" ; COc1c(-c2ncc3nc(N4CCC(NC(=O)OC(C)(C)C)C4)ccc3n2)cc2cn(C)nc2c1F ; "C102"]

A mixture of 2-(7-fluoro-6-methoxy-2-methylindazol-5-yl)pyrido[3,2-d]pyrimidin-6-ol (150 mg, 0.461 mmol, 1 equiv) and tert-butyl N-(pyrrolidin-3-yl)carbamate (103.06 mg, 0.553 mmol, 1.2 equiv) and (1H-1,2,3-benzotriazol-1-yloxy)tris(pyrrolidin-1-yl)phosphanium; hexafluoro-l\hat{ }[5]-phosphanuide (479.91 mg, 0.922 mmol, 2 equiv) and DIEA (178.79 mg, 1.383 mmol, 3 equiv) in DMF (3 mL) was stirred for 2 hr at room temperature under nitrogen atmosphere. The resulting mixture was concentrated under vacuum. The residue was purified by reverse flash chromatography (Condition 8, Gradient 1) to afford tert-butyl N-{1-[2-(7-fluoro-6-methoxy-2-methylindazol-5-yl)pyrido[3,2-d]pyrimidin-6-yl]pyrrolidin-3-yl}carbamate (150 mg, 65%) as a solid.

### Synthesis of Compounds 380 and 381

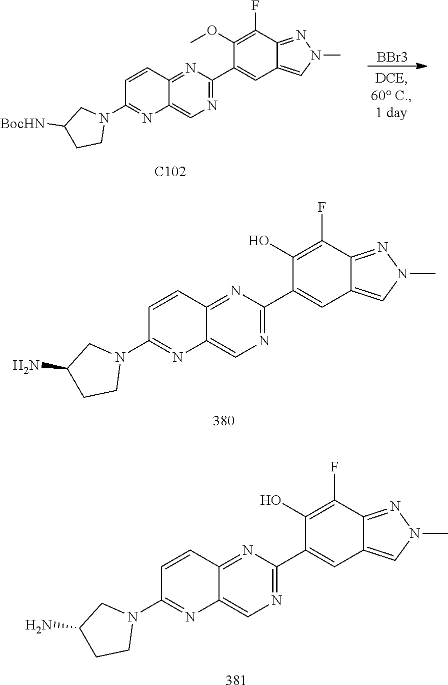

A mixture of 5-[6-(3-aminopyrrolidin-1-yl)pyrido[3,2-d]pyrimidin-2-yl]-7-fluoro-2-methylindazol-6-ol (150 mg, 0.395 mmol, 1 equiv) and boron tribromide (1 mL, 0.004 mmol, 0.01 equiv) in DCE (3 mL) was stirred for 1 day at 60° C. under nitrogen atmosphere. The reaction was quenched with methanol at 0° C. The resulting mixture was concentrated under vacuum. The crude product (150 mg) was purified by Prep-HPLC (Condition 1, Gradient 2) to afford 70 mg crude product. The product was then purified by chiral-Prep-HPLC (Condition 6, Gradient 1) to afford 5-{6-[(3R)-3-aminopyrrolidin-1-yl]pyrido[3,2-d]pyrimidin-2-yl}-7-fluoro-2-methylindazol-6-ol (4.3 mg, 2%) as a solid and 5-{6-[(3S)-3-aminopyrrolidin-1-yl]pyrido[3,2-d]pyrimidin-2-yl}-7-fluoro-2-methylindazol-6-ol (1 mg, 1%) as a solid. LCMS (ES, m/z): 380 [M+H]+ ¹H NMR (400 MHz, DMSO-d₆) δ 13.35 (s, 1H), 9.26 (d, J=2.7 Hz, 1H), 8.78 (s, 1H), 8.54 (d, J=2.7 Hz, 1H), 8.18 (d, J=9.4 Hz, 1H), 7.42 (d, J=9.1 Hz, 1H), 4.16 (d, J=3.0 Hz, 3H), 3.72 (d, J=10.4 Hz, 2H), 3.65 (s, 2H), 2.49 (s, 1H), 2.12 (s, 1H), 1.81 (s, 1H). LCMS (ES, m/z): 380 [M+H]+¹H NMR (400 MHz, DMSO-d₆) δ 13.34 (s, 1H), 9.28 (s, 1H), 8.79 (d, J=1.1 Hz, 1H), 8.54 (d, J=2.7 Hz, 1H), 8.21 (d, J=9.3 Hz, 1H), 7.44 (d, J=9.4 Hz, 1H), 4.16 (s, 3H), 3.75 (s, 4H), 3.69-3.56 (m, 1H), 2.18 (s, 1H), 1.91 (s, 1H).

### Example 145: Synthesis of Compound 383

### Synthesis of Intermediate C103

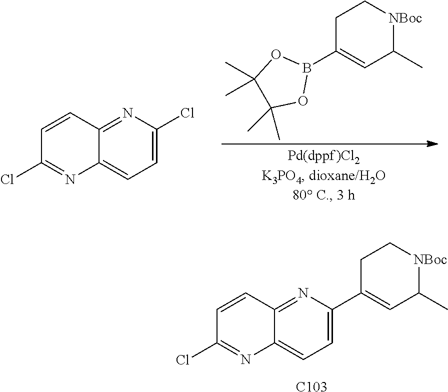

To a stirred solution of 2,6-dichloro-1,5-naphthyridine (500 mg, 2.512 mmol, 1 equiv) and tert-butyl 2-methyl-4-(4,4,5,5-tetramethyl-1,3,2-dioxaborolan-2-yl)-5,6-dihydro-2H-pyridine-1-carboxylate (1.05 g, 3.266 mmol, 1.3 equiv) in dioxane (16 mL) and H₂O (4 mL) were added Pd(dppf)Cl₂·CH₂Cl₂ (205 mg, 0.251 mmol, 0.1 equiv) and K₂CO₃ (1.04 g, 7.536 mmol, 3 equiv) in portions at room temperature. The resulting mixture was stirred for additional 3 hr at 80° C. The mixture was allowed to cool down to room temperature. The resulting mixture was extracted with ethyl acetate (3×30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (3:1) to afford tert-butyl 4-(6-chloro-1,5-naphthyridin-2-yl)-2-methyl-5,6-dihydro-2H-pyridine-1-carboxylate (500 mg, 55%) as a solid. LCMS (ES, m/z): 360 [M+H]

### Synthesis of Intermediate C104

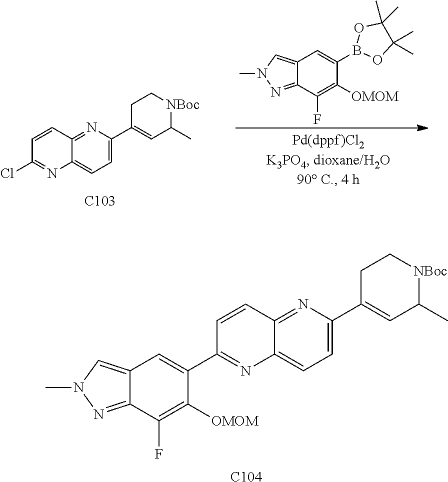

To a stirred solution of tert-butyl 4-(6-chloro-1,5-naphthyridin-2-yl)-2-methyl-5,6-dihydro-2H-pyridine-1-carboxylate (450 mg, 1.251 mmol, 1 equiv) and 7-fluoro-6-(methoxymethoxy)-2-methyl-5-(4,4,5,5-tetramethyl-1,3,2-dioxaborolan-2-yl) indazole (630 mg, 1.876 mmol, 1.5 equiv) in 1,4-dioxane (16 mL) and H₂O (4 mL) were added Pd(dppf)Cl₂·CH₂Cl₂ (102 mg, 0.125 mmol, 0.1 equiv) and K₃PO₄ (796 mg, 3.753 mmol, 3 equiv) in portions at room temperature. The resulting mixture was stirred for additional 4 hr at 90° C. The mixture was allowed to cool down to room temperature. The resulting mixture was extracted with ethyl acetate (3×30 mL). The combined organic layers were washed with water (3×30 mL), dried over anhydrous Na₂SO₄. After filtration, the filtrate was concentrated under reduced pressure. The residue was purified by silica gel column chromatography, eluted with PE/EA (1:1) to afford tert-butyl 4-{6-[7-fluoro-6-(methoxymethoxy)-2-methylindazol-5-yl]-1,5-naphthyridin-2-yl}-2-methyl-5,6-dihydro-2H-pyridine-1-carboxylate (290 mg, 43%) as a solid. LCMS (ES, m/z): 534 [M+H]

### Synthesis of Intermediate C05

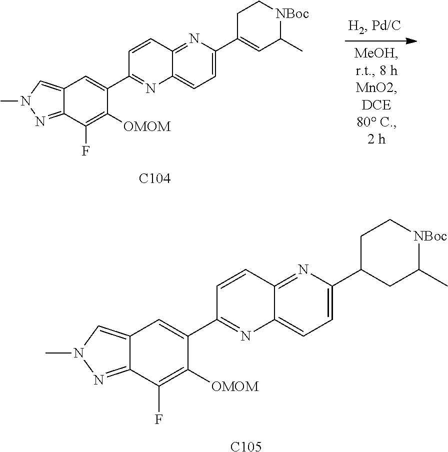

A solution of tert-butyl 4-{6-[7-fluoro-6-(methoxymethoxy)-2-methylindazol-5-yl]-1,5-naphthyridin-2-yl}-2-methyl-5,6-dihydro-2H-pyridine-1-carboxylate (260 mg, 0.487 mmol, 1.00 equiv) and Pd/C (518 mg, 4.870 mmol, 10 equiv) in methanol (30 mL) was stirred for 8 hr at room temperature under H₂ atmosphere. The resulting mixture was filtered, the filter cake was washed with methanol (3×10 mL). The filtrate was concentrated under reduced pressure. The resulting mixture and MnO₂ (424 mg, 4.870 mmol, 10 equiv) in dichloroethane (30 mL) was stirred for 2 hr at 80° C. The resulting mixture was filtered, the filter cake was washed with CH₂Cl₂ (3×10 mL). The filtrate was concentrated under reduced pressure. This resulted in tert-butyl 4-{6-[7-fluoro-6-(methoxymethoxy)-2-methylindazol-5-yl]-1,5-naphthyridin-2-yl}-2-methylpiperidine-1-carboxylate (280 mg, 96%) as a solid. LCMS (ES, m/z): 536 [M+H]

### Synthesis of Intermediate C106

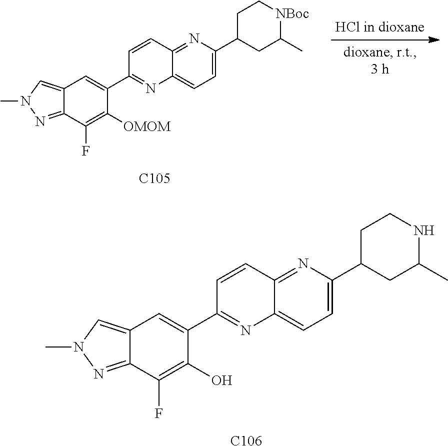

To a stirred solution of tert-butyl 4-{6-[7-fluoro-6-(methoxymethoxy)-2-methylindazol-5-yl]-1,5-naphthyridin-2-yl}-2-methylpiperidine-1-carboxylate (250 mg, 0.467 mmol, 1 equiv) in MeOH (10 mL) was added HCl(gas) in 1,4-dioxane (5 mL) dropwise at room temperature. The resulting mixture was stirred for additional 3 hr at room temperature. The resulting mixture was concentrated under vacuum. This resulted in 7-fluoro-2-methyl-5-[6-(2-methylpiperidin-4-yl)-1,5-naphthyridin-2-yl]indazol-6-ol (150 mg) as a solid. LCMS (ES, m/z): 392 [M+H]

### Synthesis of Compound 383

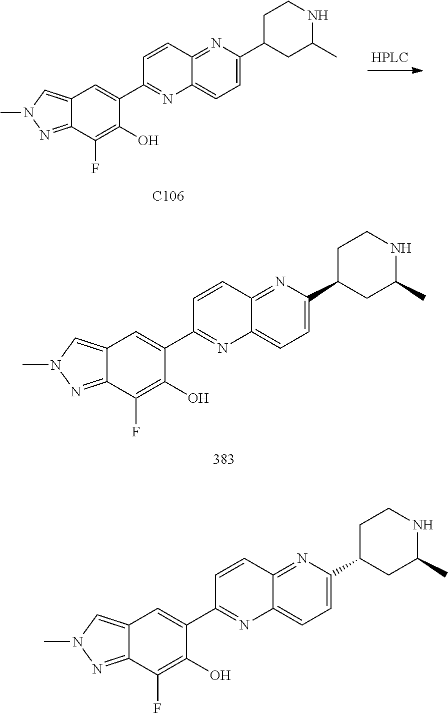

The crude product (7-fluoro-2-methyl-5-[6-(2-methylpiperidin-4-yl)-1,5-naphthyridin-2-yl]indazol-6-ol (150 mg, 0.766 mmol, 1 equiv)) was purified by Chiral Prep-HPLC (Condition 4, Gradient 3) to afford 7-fluoro-2-methyl-5-{6-[(2R,4R)-2-methylpiperidin-4-yl]-1,5-naphthyridin-2-yl}indazol-6-ol (22.6 mg, 7.53%) and 7-fluoro-2-methyl-5-{6-[(2S,4R)-2-methylpiperidin-4-yl]-1,5-naphthyridin-2-yl}indazol-6-ol (7.3 mg, 2%) as a solid. Analysis data

| Compound Structure | Analysis data |
|---|---|
| 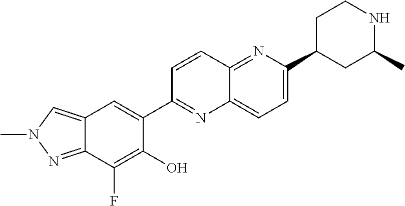 | LCMS (ES, m/z): 392 [M + H] ¹H NMR (400 MHz, DMSO-d₆) δ 14.08 (s, 1H), 8.63 (d, J = 9.3 Hz, 1H), 8.57 (dd, J = 9.0, 1.9 Hz, 2H), 8.54-8.38 (m, 1H), 8.46 (s, 1H), 7.79 (d, J = 8.7 Hz, 1H), 4.18 (s, 3H), 3.17- 3.01 (m, 2H), 2.80-2.77 (m, 2H), 1.91 (ddd, J = 15.1, 9.2, 6.8 Hz, 2H), 1.69 (qd, J = 12.4, 4.1 Hz, 1H), 1.43 (q, J = 12.0 Hz, 1H), 1.08 (d, J = 6.3 Hz, 3H). ¹⁹F NMR (376 MHz, DMSO-d₆) δ −157.95. |
| 383 | |
| 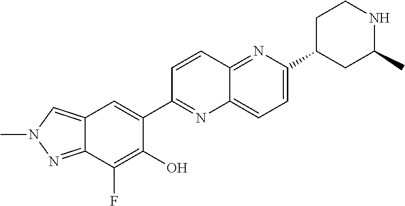 | LCMS (ES, m/z): 392 [M + H] ¹H NMR (400 MHz, DMSO-d₆) δ 14.07 (s, 1H), 8.64 (d, J = 9.3 Hz, 1H), 8.58 (d, J = 1.0 Hz, 1H), 8.56 (d, J = 2.7 Hz, 1H), 8.52 (dd, J = 9.1, 0.8 Hz, 1H), 8.47 (dd, J = 8.6, 0.9 Hz, 1H), 7.84 (d, J = 8.8 Hz, 1H), 4.18 (s, 3H), 3.48- 3.41 (m, 1H), 3.01-2.90 (m, 1H), 2.96 (s, 2H), 2.18 (ddd, J = 13.4, 7.6, 3.9 Hz, 1H), 2.11- 2.01 (m, 1H), 1.93 (d, J = 11.4 Hz, 1H), 1.73 (dt, J = 12.9, 5.8 Hz, 1H), 1.18 (d, J = 6.6 Hz, 3H). ¹⁹F NMR (376 MHz, DMSO-d₆) δ −157.94. |

### Example 146: Synthesis of Compound 298

### Synthesis of Intermediate C107

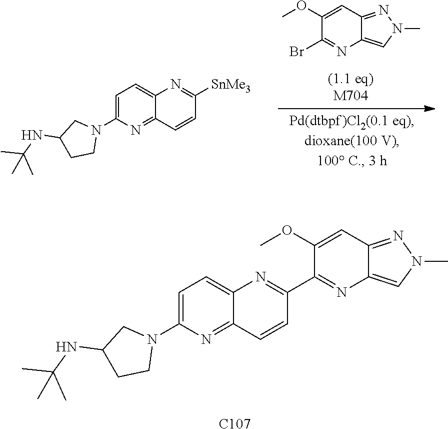

Into a 25 mL vial were added N-tert-butyl-1-[6-(trimethylstannyl)-1,5-naphthyridin-2-yl]pyrrolidin-3-amine (100 mg, 0.231 mmol, 1 equiv), 5-bromo-6-methoxy-2-methylpyrazolo[4,3-b]pyridine (55.88 mg, 0.231 mmol, 1 equiv), dioxane (10 mL) and Pd(DtBPF)Cl₂ (15.05 mg, 0.023 mmol, 0.1 equiv) at room temperature. The resulting mixture was stirred for 3 hr at 100° C. under nitrogen atmosphere. The mixture was allowed to cool down to room temperature. The reaction was quenched by the addition of water/KF (20 mL) at room temperature. The resulting mixture was extracted with ethyl acetate (3×10 mL). The combined organic layers were washed with brine (1×20 mL), dried over anhydrous Na₂SO₄. After filtration, the filtrate was concentrated under reduced pressure. The residue was purified by Prep-TLC (CH₂Cl₂/MeOH 10:1) to afford N-tert-butyl-1-(6-{6-methoxy-2-methylpyrazolo[4,3-b]pyridin-5-yl}-1,5-naphthyridin-2-yl) pyrrolidin-3-amine (50 mg, 50%) as a solid. LCMS (ES, m/z):432 [M+H]⁺

### Synthesis of Compound 298

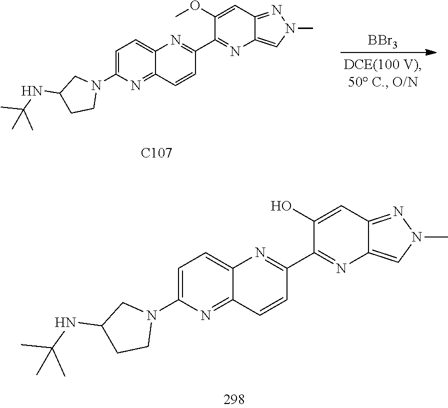

Into a 25 mL vial were added N-tert-butyl-1-(6-{6-methoxy-2-methylpyrazolo[4,3-b]pyridin-5-yl}-1,5-naphthyridin-2-yl)pyrrolidin-3-amine (50 mg, 0.116 mmol, 1 equiv), DCE (5 mL) and BBr₃ (1 mL) at room temperature. The resulting mixture was stirred for overnight at 50° C. The reaction was quenched by the addition of methanol (5 mL) at 0° C. The resulting mixture was concentrated under reduced pressure. The mixture was basified to pH 8 with saturated NaHCO₃ (aq.). The aqueous layer was extracted with CH₂Cl₂ (3×10 mL). The combined organic layers were washed with brine (1×15 mL), dried over anhydrous Na₂SO₄. After filtration, the filtrate was concentrated under reduced pressure. The crude product (45 mg) was purified by Prep-HPLC (Condition 1, Gradient 28) to afford 5-{6-[3-(tert-butylamino)pyrrolidin-1-yl]-1,5-naphthyridin-2-yl}-2-methylpyrazolo[4,3-b]pyridin-6-ol (9.9 mg, 20%) as a solid. LCMS (ES, m/z):418 [M+H]^{+ 1}H NMR (400 MHz, DMSO-d₆) δ 14.20 (s, 1H), 8.75 (d, J=9.0 Hz, 1H), 8.66 (s, 1H), 8.22-8.10 (m, 2H), 7.35 (d, J=1.0 Hz, 1H), 7.19 (d, J=9.3 Hz, 1H), 4.18 (s, 3H), 3.88 (s, 1H), 3.73 (s, 1H), 3.59-3.45 (m, 2H), 3.23-3.09 (m, 1H), 2.19 (s, 1H), 1.76 (s, 2H), 1.10 (s, 9H).

### Example 147: Synthesis of Compounds 347 and 349

### Synthesis of Intermediate C108

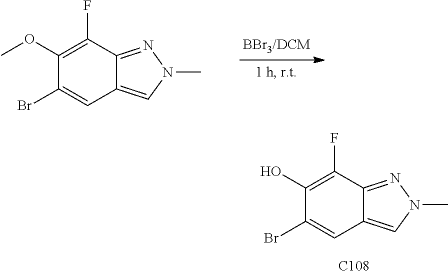

Into a 250 mL 3-necked round-bottom flask was added to 5-bromo-7-fluoro-6-methoxy-2-methyl-2H-indazole (5 g, 19.37 mmol, 2.20 equiv) and DCE (50.0 mL). Then BBr₃ (60 mL, 3 equiv, 1 M in dioxane) was added dropwise to the mixture at 0° C. in 10 min. And the temperature was warmly to room temperature and the resulting solution was stirred for 1 h at room temperature. The reaction was then quenched by the addition of 100 mL of methanol at 0° C. and the solvent were removed in vacuo. The pH value of the solution was adjusted to 7 with saturated NaHCO₃. The resulting solution was extracted with 3×100 mL of ethyl acetate and the organic layers combined and dried over anhydrous sodium sulfate and concentrated. This resulted in 4.3 g (crude) of 5-bromo-7-fluoro-2-methyl-2H-indazol-6-ol as a solid which was used directly in the subsequent step.

### Synthesis of Intermediate C109

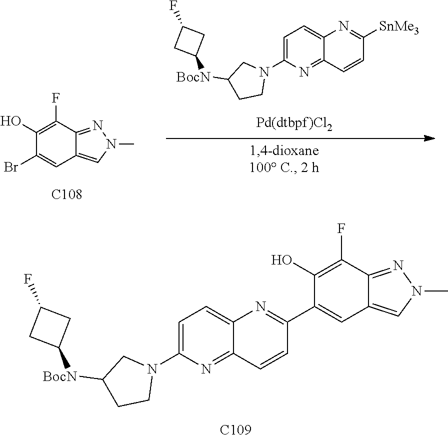

Into a 40 mL vial purged and maintained with an inert atmosphere of nitrogen, was placed 5-bromo-7-fluoro-2-methyl-2H-indazol-6-ol (235 mg, 0.96 mmol, 1.00 equiv), tert-butyl ((1r,3r)-3-fluorocyclobutyl)(1-(6-(trimethylstannyl)-1,5-naphthyridin-2-yl)pyrrolidin-3-yl)carbamate (528 mg, 0.960 mmol, 1.00 equiv), Pd(dtbpf)Cl₂ (62 mg, 0.0960 mmol, 0.1 equiv) and 1,4-dioxane (20 mL). The resulting solution was stirred for 2 hr at 100° C. After that the reaction was colder at room temperature and was extracted with 3×20 mL of ethyl acetate. The organic layers combined and dried over anhydrous sodium sulfate and concentrated. The residue was applied onto a silica gel column with ethyl acetate/hexane (1:1). This resulted in 100.0 mg as a solid that was used in the following step without further purification. LCMS (ES, m/z): 551 [M+H]⁺

### Synthesis of Intermediate C110

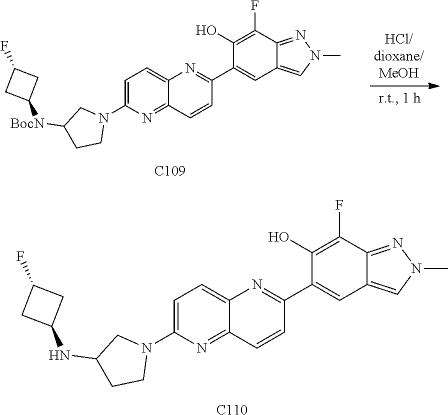 [SMILES: Cn1cc2cc(-c3ccc4nc(N5CCC(N(C(=O)OC(C)(C)C)[C@H]6C[C@H](F)C6)C5)ccc4n3)c(O)c(F)c2n1 ; "C109" ; Cn1cc2cc(-c3ccc4nc(N5CCC(N[C@H]6C[C@H](F)C6)C5)ccc4n3)c(O)c(F)c2n1 ; "C110"]

Into a 40-mL round-bottom was placed tert-butyl (1-(6-(7-fluoro-6-hydroxy-2-methyl-2H-indazol-5-yl)-1,5-naphthyridin-2-yl)pyrrolidin-3-yl)((1r,3r)-3-fluorocyclobutyl)carbamate (100 mg, 0.182 mmol, 1.00 equiv), MeOH (1 mL), HCl (g) in dioxane (3.00 mL, 1 mol/L, 3 equiv.). The resulting solution was stirred for 1 h at room temperature. The resulting mixture was concentrated. The crude product was purified by prep-HPLC (Condition 3, Gradient 22) to afford 7-fluoro-5-(6-(3-(((1r,3r)-3-fluorocyclobutyl) amino)pyrrolidin-1-yl)-1,5-naphthyridin-2-yl)-2-methyl-2H-indazol-6-ol (50 mg, 63%) as a solid. LCMS (ES, m/z): 451 [M+H]+

### Synthesis of Compounds 347 and 349

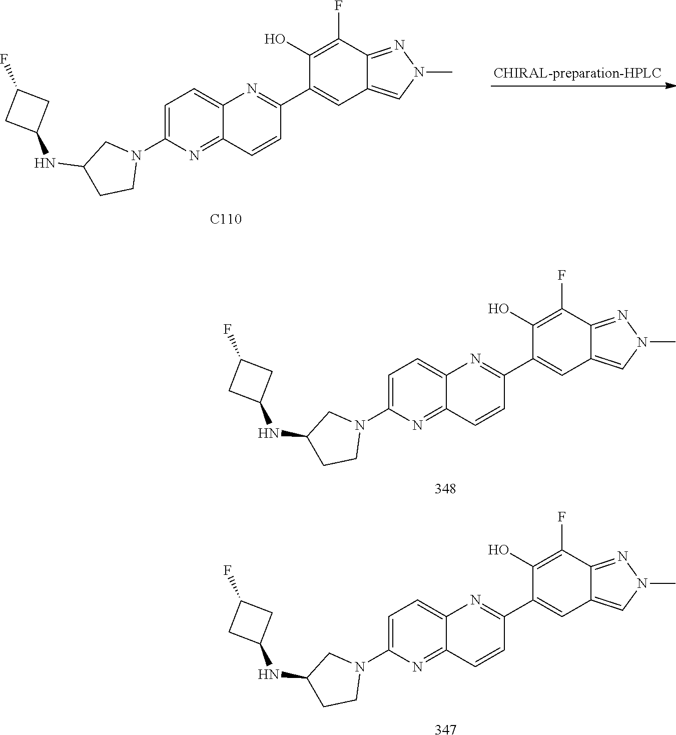 [SMILES: Cn1cc2cc(-c3ccc4nc(N5CCC(N[C@H]6C[C@H](F)C6)C5)ccc4n3)c(O)c(F)c2n1 ; "C110" ; Cn1cc2cc(-c3ccc4nc(N5CC[C@@H](N[C@H]6C[C@H](F)C6)C5)ccc4n3)c(O)c(F)c2n1 ; "348" ; Cn1cc2cc(-c3ccc4nc(N5CC[C@@H](N[C@H]6C[C@H](F)C6)C5)ccc4n3)c(O)c(F)c2n1 ; "347"]

7-fluoro-5-(6-(3-(((1 r,3r)-3-fluorocyclobutyl)amino)pyrrolidin-1l-yl)-1,5-naphthyridin-2-yl)-2-methyl-2H-indazol-6-ol (52 mg) was purified by Chiral prep-HPLC (Condition 4, Gradient 6) to afford 7-fluoro-5-(6-((R)-3-(((1r,3R)-3-fluorocyclobutyl)amino)pyrrolidin-1l-yl)-1,5-naphthyridin-2-yl)-2-methyl-2H-indazol-6-ol (9.9 mg, 14%) and 7-fluoro-5-(6-((R)-3-(((1r,3R)-3-fluorocyclobutyl)amino)pyrrolidin-1l-yl)-1,5-naphthyridin-2-yl)-2-methyl-2H-indazol-6-ol (10.7 mg, 14%). LCMS (ES, m/z). 451 [M+H]⁺ Compound 348: ¹H NMR (400 MHz, DMSO-d₆) δ 14.40 (s, 1H), 8.48 (d, J=2.7 Hz, 1H), 8.44-8.38 (m, 2H), 8.16 (d, J=9.3 Hz, 1H), 8.09 (dd, J=9.1, 0.8 Hz, 1H), 7.19 (d, J=9.3 Hz, 1H), 5.21 (ddd, J=59.9, 6.3, 3.0 Hz, 1H), 4.16 (s, 3H), 3.73 (d, J=25.1 Hz, 2H), 3.65-3.52 (m, 2H), 3.19-2.73 (m, 1H), 2.47-2.31 (m, 2H), 2.30-2.09 (m, 3H), 1.97-1.83 (m, 1H). Compound 347: ¹H NMR (400 MHz, DMSO-d₆) δ 14.40 (s, 1H), 8.48 (d, J=2.6 Hz, 1H), 8.44-8.38 (m, 2H), 8.15 (d, J=9.3 Hz, 1H), 8.09 (dd, J=9.1, 0.8 Hz, 1H), 7.18 (d, J=9.3 Hz, 1H), 5.35-5.05 (m, 1H), 4.16 (s, 3H), 3.76 (dd, J=10.2, 5.6 Hz, 2H), 3.73-3.57 (m, 2H), 3.56-3.51 (m, 1H), 2.97-2.88 (m, 1H), 2.46-2.35 (m, 2H), 2.30-2.10 (m, 3H), 1.97-1.81 (m, 1H).

### Example 148: Synthesis of Compound 374

### Synthesis of Intermediate C111

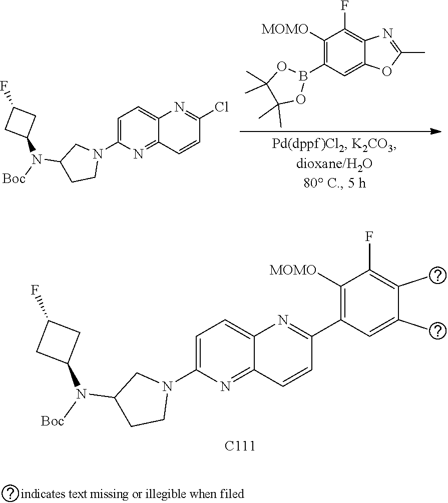

To a solution of tert-butyl N-[1-(6-chloro-1,5-naphthyridin-2-yl)pyrrolidin-3-yl]-N-[(1r,3r)-3-fluorocyclobutyl]carbamate (70 mg, 0.166 mmol, 1 equiv) and 4-fluoro-5-(methoxymethoxy)-2-methyl-6-(4,4,5,5-tetramethyl-1,3,2-dioxaborolan-2-yl)-1,3-benzoxazole (112.14 mg, 0.332 mmol, 2 equiv) in 1,4-dioxane (1 mL) and H₂O (0.25 mL) were added K₂CO₃ (68.95 mg, 0.498 mmol, 3 equiv) and Pd(dppf)Cl₂ (12.17 mg, 0.017 mmol, 0.1 equiv). After stirring for 5 hr at 80° C. under a nitrogen atmosphere, the resulting mixture was concentrated under reduced pressure. The residue was purified by Prep-TLC/silica gel column chromatography, eluted with CH₂Cl₂/MeOH (9:1) to afford tert-butyl N-(1-{6-[4-fluoro-5-(methoxymethoxy)-2-methyl-1,3-benzoxazol-6-yl]-1,5-naphthyridin-2-yl}pyrrolidin-3-yl)-N-[(1r,3r)-3-fluorocyclobutyl]carbamate (30 mg, 30%) as a solid.

### Synthesis of Compound 374

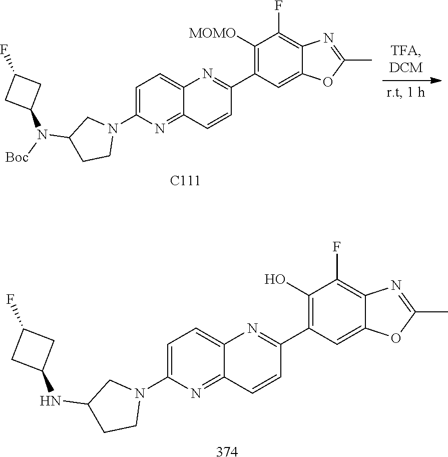

To a solution of tert-butyl N-(1-{6-[4-fluoro-5-(methoxymethoxy)-2-methyl-1,3-benzoxazol-6-yl]-1,5-naphthyridin-2-yl}pyrrolidin-3-yl)-N-[(1r,3r)-3-fluorocyclobutyl]carbamate (30 mg, 0.050 mmol, 1 equiv) in DCM (1 mL) was added TFA (0.2 mL). After stirring for 1 hr at room temperature under a nitrogen atmosphere, the resulting mixture was concentrated under reduced pressure. The crude product was purified by Prep-HPLC with the following conditions (Condition 1, Gradient 25) to afford 4-fluoro-2-methyl-6-[6-(3-{[(1r,3r)-3-fluorocyclobutyl]amino}pyrrolidin-1-yl)-1,5-naphthyridin-2-yl]-1,3-benzoxazol-5-ol (1.6 mg, 7%) as a solid. LCMS (ES, m/z): 452 [M+H]+ ¹H NMR (400 MHz, DMSO-d₆) δ 15.06 (s, 1H), 8.45 (d, J=9.2 Hz, 1H), 8.33 (d, J=1.4 Hz, 1H), 8.14 (dd, J=22.9, 9.2 Hz, 2H), 7.19 (d, J=9.3 Hz, 1H), 5.19 (dtt, J=56.9, 6.5, 3.6 Hz, 1H), 3.72 (s, 2H), 3.55 (s, 2H), 3.34-3.37 (m, 1H) 2.64 (s, 3H), 2.34-2.43 (m, 3H), 2.14-2.29 (m, 3H), 1.87 (d, J=31.1 Hz, 1H).

### Example 149: Synthesis of Compounds 397, 398, and 399

### Synthesis of Intermediate C112

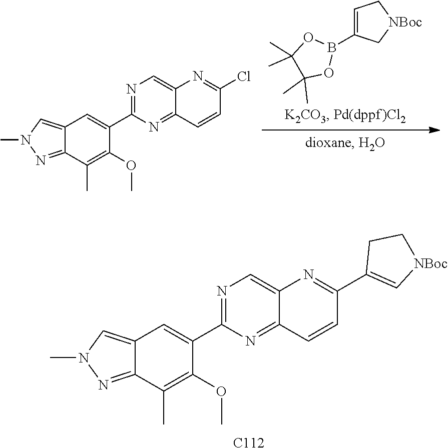

To a stirred solution of 5-{6-chloropyrido[3,2-d]pyrimidin-2-yl}-6-methoxy-2,7-dimethylindazole (200 mg, 0.589 mmol, 1 equiv) and tert-butyl 3-(4,4,5,5-tetramethyl-1,3,2-dioxaborolan-2-yl)-2,5-dihydropyrrole-1-carboxylate (174 mg, 0.589 mmol, 1 equiv) in dioxane (5 mL) and H₂O (1 mL) were added K₂CO₃ (203 mg, 1.472 mmol, 2.5 equiv) and Pd(dppf)Cl₂ (43 mg, 0.059 mmol, 0.1 equiv) in portions at room temperature under nitrogen atmosphere. The resulting mixture was stirred for 3 hr at 80° C. under nitrogen atmosphere. The mixture was allowed to cool down to room temperature. The reaction was quenched by the addition of water (20 mL) at room temperature. The resulting mixture was extracted with ethyl acetate (3×5 mL). The combined organic layers were washed with brine (1×10 mL), dried over anhydrous Na₂SO₄. After filtration, the filtrate was concentrated under reduced pressure. The residue was purified by reverse flash chromatography (Condition 7, Gradient 4) to afford tert-butyl 3-[2-(6-methoxy-2,7-dimethylindazol-5-yl)pyrido[3,2-d]pyrimidin-6-yl]-4,5-dihydropyrrole-1-carboxylate (187 mg, 67%) as a solid. LCMS (ES, m/z): 473 [M+H]⁺

### Synthesis of Intermediate C113

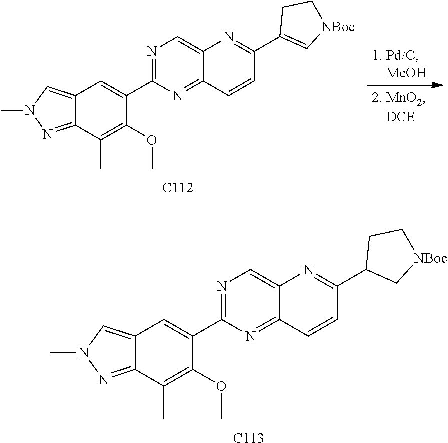 [SMILES: COc1c(-c2ncc3nc(C4=CN(C(=O)OC(C)(C)C)CC4)ccc3n2)cc2cn(C)nc2c1C ; "C112" ; COc1c(-c2ncc3nc(C4CCN(C(=O)OC(C)(C)C)C4)ccc3n2)cc2cn(C)nc2c1C ; "C113"]

To a solution of tert-butyl 3-[2-(6-methoxy-2,7-dimethylindazol-5-yl)pyrido[3,2-d]pyrimidin-6-yl]-4,5-dihydropyrrole-1-carboxylate (187 mg, 0.396 mmol, 1 equiv) in ethyl acetate (5 mL) was added Pd/C (42 mg, 0.396 mmol, 1 equiv) under nitrogen atmosphere. The mixture was hydrogenated at room temperature for 5 hr under hydrogen atmosphere using a hydrogen balloon, filtered through a Celite pad and concentrated under reduced pressure. The residue was dissolve in DCE (5 mL) and added MnO₂ (301 mg, 3.460 mmol, 10 equiv) in portions at room temperature. The resulting mixture was stirred for 4 hr at 60° C. The mixture was allowed to cool down to room temperature. The resulting mixture was filtered. the filter cake was washed with CH₂Cl₂ (3×5 mL). The filtrate was concentrated under reduced pressure to afford tert-butyl 3-[2-(6-methoxy-2,7-dimethylindazol-5-yl)pyrido[3,2-d]pyrimidin-6-yl]pyrrolidine-1-carboxylate (135 mg, 82%) as a solid. LCMS (ES, m/z): 475 [M+H]⁺

### Synthesis of Compound 397

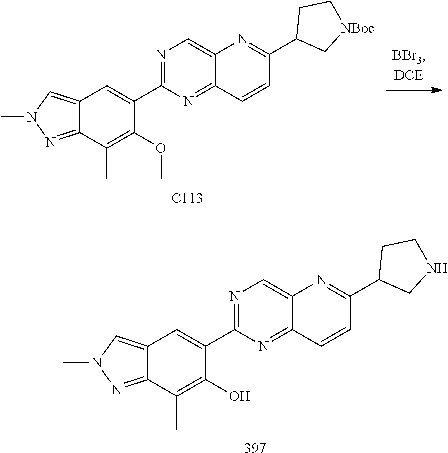 [SMILES: COc1c(-c2ncc3nc(C4CCN(C(=O)OC(C)(C)C)C4)ccc3n2)cc2cn(C)nc2c1C ; "C113" ; Cc1c(O)c(-c2ncc3nc(C4CCNC4)ccc3n2)cc2cn(C)nc12 ; "397"]

To a stirred solution of tert-butyl 3-[2-(6-methoxy-2,7-dimethylindazol-5-yl)pyrido[3,2-d]pyrimidin-6-yl]pyrrolidine-1-carboxylate (130 mg, 0.274 mmol, 1 equiv) in DCE (5 mL) was added BBr₃ (343 mg, 1.370 mmol, 5 equiv) dropwise at room temperature. The resulting mixture was stirred for 0.5 hr at 80° C. The mixture was allowed to cool down to room temperature. The reaction was quenched by the addition of water (5 mL) at room temperature. The resulting mixture was concentrated under vacuum. The residue was purified by reverse flash (Condition 4, Gradient 2) to afford 2,7-dimethyl-5-[6-(pyrrolidin-3-yl)pyrido[3,2-d]pyrimidin-2-yl]indazol-6-ol; trifluoroacetic acid (20 mg, 15%) as a solid. LCMS (ES, m/z): 361 [M+H]^{+ 1}H NMR (300 MHz, Methanol-d₄) δ 9.65 (d, J=0.8 Hz, 1H), 9.11 (d, J=0.7 Hz, 1H), 8.48-8.31 (m, 2H), 7.98 (d, J=8.8 Hz, 1H), 4.31-4.18 (m, 1H), 4.24 (s, 3H), 4.10 (ddd, J=13.2, 7.5, 5.8 Hz, 1H), 3.97 (dd, J=11.8, 5.7 Hz, 1H), 3.74 (dd, J=11.7, 7.4 Hz, 1H), 3.66-3.45 (m, 2H), 2.75-2.56 (m, 1H), 2.60-2.37 (m, 3H), 2.34 (dt, J=13.4, 6.9 Hz, 1H).

### Synthesis of Compounds 398 and 399

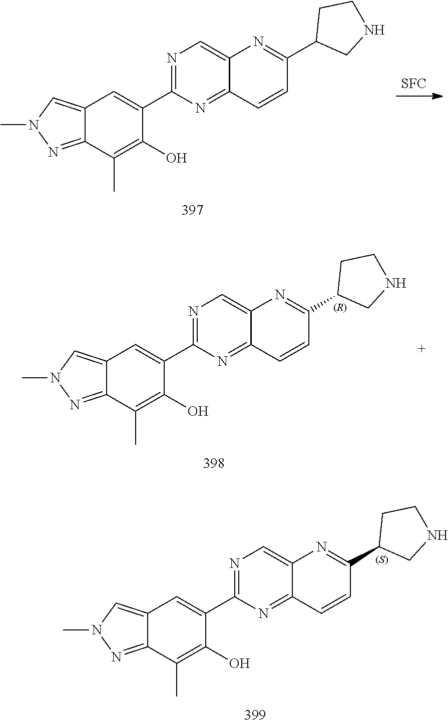

2,7-dimethyl-5-[6-(pyrrolidin-3-yl)pyrido[3,2-d]pyrimidin-2-yl]indazol-6-ol; trifluoroacetic acid was separated Chiral-Prep-HPLC (Condition 7, Gradient 1) to afford (R)-2,7-dimethyl-5-(6-(pyrrolidin-3-yl)pyrido[3,2-d]pyrimidin-2-yl)-2H-indazol-6-ol (7.3 mg, 43%) as a solid and (S)-2-ethyl-4-(3-(ethylamino)pyrrolidin-1-yl)-N-(8-fluoro-2-methylimidazo[1,2-a]pyridin-6-yl)-2H-indazole-7-carboxamide (3.1 mg, 18%) as a solid. Compound 398: RT=2.824 min on chiral-HPLC LCMS (ES, m/z): 361 [M+H]^{+ 1}H NMR (300 MHz, Methanol-d₄) δ 9.58 (s, 1H), 9.02 (s, 1H), 8.36-8.19 (m, 2H), 7.92 (d, J=8.8 Hz, 1H), 4.19 (s, 3H), 3.81 (q, J=7.3 Hz, 1H), 3.45 (d, J=7.2 Hz, 2H), 3.32-3.13 (m, 1H), 2.45 (s, 3H), 2.43 (s, 1H), 2.24 (dq, J=14.0, 7.1 Hz, 1H). Compound 399: RT=4.399 min on chiral-HPLC LCMS (ES, m/z): 361 [M+H]^{+ 1}H NMR (300 MHz, Methanol-d₄) δ 9.58 (s, 1H), 9.02 (s, 1H), 8.36-8.19 (m, 2H), 7.92 (d, J=8.8 Hz, 1H), 4.19 (s, 3H), 3.81 (q, J=7.3 Hz, 1H), 3.45 (d, J=7.2 Hz, 2H), 3.32-3.13 (m, 1H), 2.45 (s, 3H), 2.43 (s, 1H), 2.24 (dq, J=14.0, 7.1 Hz, 1H).

### Example 150: Synthesis of Compound 455

### Synthesis of Intermediate C114

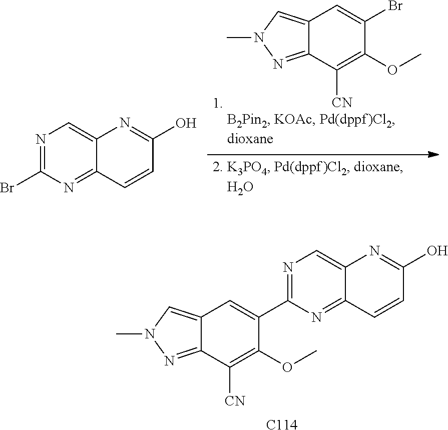

To a stirred solution of 5-bromo-6-methoxy-2-methylindazole-7-carbonitrile (589 mg, 2.212 mmol, 1 equiv) and 4,4,5,5-tetramethyl-2-(tetramethyl-1,3,2-dioxaborolan-2-yl)-1,3,2-dioxaborolane (674 mg, 2.654 mmol, 1.2 equiv) in dioxane (20 mL) were added KOAc (434 mg, 4.424 mmol, 2 equiv) and Pd(dppf)Cl₂ (162 mg, 0.221 mmol, 0.1 equiv) in portions at room temperature under nitrogen atmosphere. The resulting mixture was stirred for 3 hr at 90° C. under nitrogen atmosphere. The mixture was allowed to cool down to room temperature. To the above mixture was added 2-bromopyrido[3,2-d]pyrimidin-6-ol (500 mg, 2.212 mmol, 1 equiv), Pd(dppf)Cl₂ (162 mg, 0.221 mmol, 0.1 equiv), K₃PO₄ (939 mg, 4.424 mmol, 2 equiv) and H₂O (2 mL) in portions at room temperature. The resulting mixture was stirred for additional 3 hr at 90° C. The mixture was allowed to cool down to room temperature. The resulting mixture was concentrated under reduced pressure. The residue was purified by silica gel column chromatography, eluted with CH₂Cl₂/MeOH (10:1) to afford 5-{6-hydroxypyrido[3,2-d]pyrimidin-2-yl}-6-methoxy-2-methylindazole-7-carbonitrile (110 mg, 15%) as a solid. LCMS (ES, m/z): 333 [M+H]⁺

### Synthesis of Intermediate C115

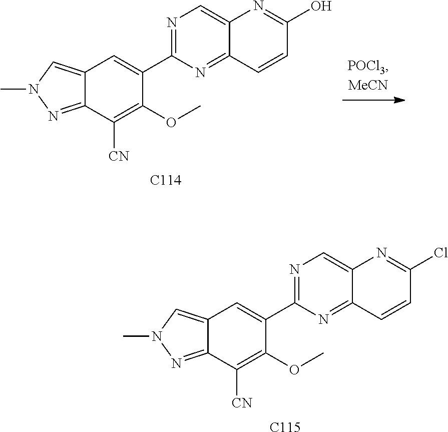 [SMILES: COc1c(-c2ncc3nc(O)ccc3n2)cc2cn(C)nc2c1C#N ; "C114" ; COc1c(-c2ncc3nc(Cl)ccc3n2)cc2cn(C)nc2c1C#N ; "C115"]

To a stirred solution of 5-{6-hydroxypyrido[3,2-d]pyrimidin-2-yl}-6-methoxy-2-methylindazole-7-carbonitrile (110 mg, 0.331 mmol, 1 equiv) in MeCN (5 mL) was added POCl₃ (508 mg, 3.310 mmol, 10 equiv) dropwise at room temperature. The resulting mixture was stirred for 3 h at 80° C. The mixture was allowed to cool down to room temperature. The resulting mixture was concentrated under vacuum. The residue was dissolved in ethyl acetate (20 mL). The mixture was basified to pH 8 with saturated NaHCO₃ (aq.). The resulting mixture was extracted with ethyl acetate (3×10 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (1:10) to afford 5-{6-chloropyrido[3,2-d]pyrimidin-2-yl}-6-methoxy-2-methylindazole-7-carbonitrile (56 mg, 48%) as a solid. LCMS (ES, m/z): 351 [M+H]⁺

### Synthesis of Intermediate C116

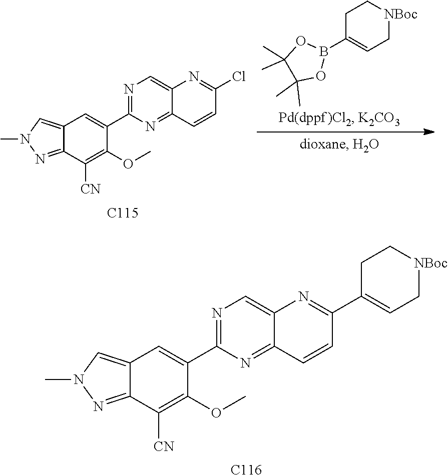

To a stirred solution of 5-{6-chloropyrido[3,2-d]pyrimidin-2-yl}-6-methoxy-2-methylindazole-7-carbonitrile (55 mg, 0.157 mmol, 1 equiv) and tert-butyl 4-(4,4,5,5-tetramethyl-1,3,2-dioxaborolan-2-yl)-3,6-dihydro-2H-pyridine-1-carboxylate (58 mg, 0.188 mmol, 1.2 equiv) in dioxane (2 mL) and H₂O (0.2 mL) were added K₂CO₃ (54 mg, 0.393 mmol, 2.5 equiv) and Pd(dppf)Cl₂ (12 mg, 0.016 mmol, 0.1 equiv) in portions at room temperature under nitrogen atmosphere. The resulting mixture was stirred for 4 h at 90° C. under nitrogen atmosphere. The mixture was allowed to cool down to room temperature. The reaction was quenched by the addition of water (20 mL) at room temperature. The resulting mixture was extracted with ethyl acetate (3×5 mL). The combined organic layers were washed with brine (3×10 mL), dried over anhydrous Na₂SO₄. After filtration, the filtrate was concentrated under reduced pressure. The residue was purified by silica gel column chromatography, eluted with PE/EA (1:10) to afford tert-butyl 4-[2-(7-cyano-6-methoxy-2-methylindazol-5-yl)pyrido[3,2-d]pyrimidin-6-yl]-3,6-dihydro-2H-pyridine-1-carboxylate (58 mg, 74%) as a solid. LCMS (ES, m/z): 498 [M+H]⁺

### Synthesis of Intermediate C117

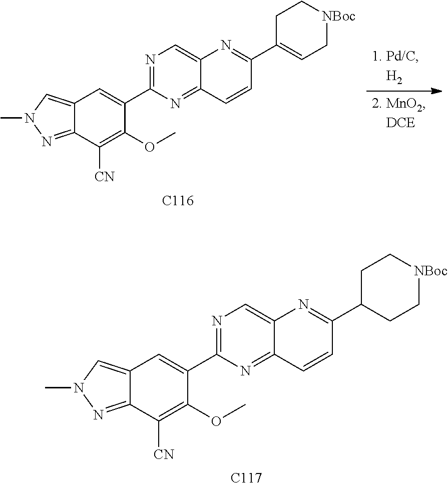 [SMILES: COc1c(-c2ncc3nc(C4=CCN(C(=O)OC(C)(C)C)CC4)ccc3n2)cc2cn(C)nc2c1C#N ; "C116" ; COc1c(-c2ncc3nc(C4CCN(C(=O)OC(C)(C)C)CC4)ccc3n2)cc2cn(C)nc2c1C#N ; "C117"]

To a solution of tert-butyl 4-[2-(7-cyano-6-methoxy-2-methylindazol-5-yl)pyrido[3,2-d]pyrimidin-6-yl]-3,6-dihydro-2H-pyridine-1-carboxylate (51 mg, 0.103 mmol, 1 equiv) in MeOH (5 mL) was added Pd/C (55 mg, 0.515 mmol, 5 equiv) under nitrogen atmosphere. The mixture was hydrogenated at room temperature for 5 hr under hydrogen atmosphere using a hydrogen balloon, filtered through a Celite pad and concentrated under reduced pressure. The residue was diluted with DCE (5 mL). To the above mixture was added MnO₂ (89 mg, 1.030 mmol, 10 equiv) in portions at room temperature. The resulting mixture was stirred for additional 4 h at 60° C. The mixture was allowed to cool down to room temperature. The resulting mixture was filtered; the filter cake was washed with DCE (3×10 mL). The filtrate was concentrated under reduced pressure to afford tert-butyl 4-[2-(7-cyano-6-methoxy-2-methylindazol-5-yl)pyrido[3,2-d]pyrimidin-6-yl]piperidine-1-carboxylate (50 mg, 97%) as a solid. LCMS (ES, m/z): 500 [M+H]⁺

### Synthesis of Compound 455

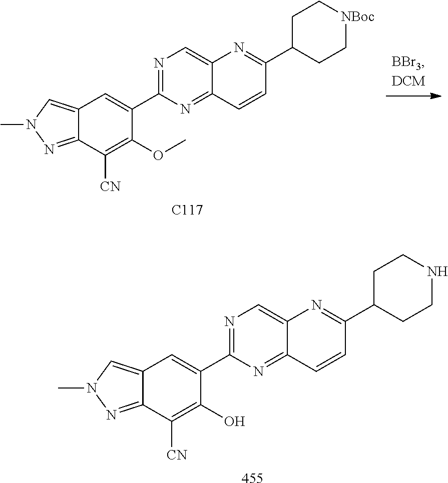 [SMILES: COc1c(-c2ncc3nc(C4CCN(C(=O)OC(C)(C)C)CC4)ccc3n2)cc2cn(C)nc2c1C#N ; "C117" ; Cn1cc2cc(-c3ncc4nc(C5CCNCC5)ccc4n3)c(O)c(C#N)c2n1 ; "455"]

To a stirred solution of tert-butyl 4-[2-(7-cyano-6-methoxy-2-methylindazol-5-yl)pyrido[3,2-d]pyrimidin-6-yl]piperidine-1-carboxylate (60 mg, 0.12 mmol, 1 equiv) in DCE (5 mL) was added BBr₃ (301 mg, 1.2 mmol, 10 equiv) dropwise at room temperature. The resulting mixture was stirred for 0.5 hr at 80° C. The mixture was allowed to cool down to room temperature. The resulting mixture was concentrated under vacuum. The residue was purified by reverse flash chromatography (Condition 4, Gradient 3) to afford 6-hydroxy-2-methyl-5-[6-(piperidin-4-yl)pyrido[3,2-d]pyrimidin-2-yl]indazole-7-carbonitrile; trifluoroacetic acid (13 mg, 21%) as a solid. LCMS (ES, m/z): 386 [M+H]^{+ 1}H NMR (300 MHz, Methanol-d₄) δ 9.67 (s, 1H), 9.46 (s, 1H), 8.54-8.42 (m, 2H), 8.02 (d, J=8.8 Hz, 1H), 4.23 (s, 3H), 3.62 (d, J=12.9 Hz, 2H), 3.45 (s, 1H), 3.26 (d, J=12.8 Hz, 2H), 2.43-2.19 (m, 4H).

### Example 151: Synthesis of Compound 371

### Synthesis of Intermediate C118

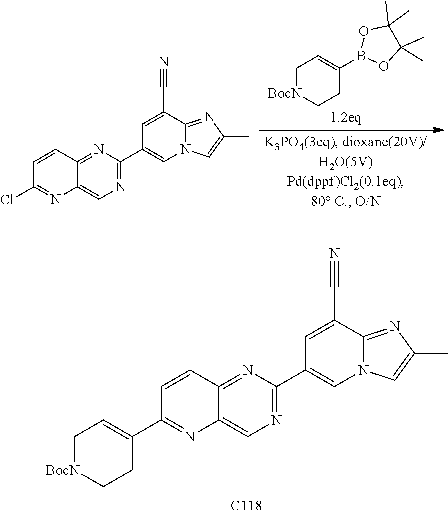 [SMILES: Cc1cn2cc(-c3ncc4nc(Cl)ccc4n3)cc(C#N)c2n1 ; CC(C)(C)OC(=O)N1CC=C(B2OC(C)(C)C(C)(C)O2)CC1 ; Cc1cn2cc(-c3ncc4nc(C5=CCN(C(=O)OC(C)(C)C)CC5)ccc4n3)cc(C#N)c2n1 ; "C118"]

A solution of 6-{6-chloropyrido[3,2-d]pyrimidin-2-yl}-2-methylimidazo[1,2-a]pyridine-8-carbonitrile (500.0 mg, 1.56 mmol, 1 equiv), tert-butyl 4-(4,4,5,5-tetramethyl-1,3,2-dioxaborolan-2-yl)-3,6-dihydro-2H-pyridine-1-carboxylate (578.4 mg, 1.87 mmol, 1.2 equiv), K₃PO₄ (992.7 mg, 4.67 mmol, 3 equiv) and Pd(dppf)Cl₂CH₂Cl₂ (127.0 mg, 0.15 mmol, 0.1 equiv) in dioxane (8 mL) and H₂O (2 mL) was stirred for overnight at 80° C. under nitrogen atmosphere. The resulting mixture was diluted with water (20 mL). The resulting mixture was extracted with ethyl acetate (3×20 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (1:4) to afford tert-butyl 4-(2-{8-cyano-2-methylimidazo[1,2-a]pyridin-6-yl}pyrido[3,2-d]pyrimidin-6-yl)-3,6-dihydro-2H-pyridine-1-carboxylate (120.0 mg, 16%) as a solid. LCMS (ES, m/z): 468 [M+H]⁺

### Synthesis of Intermediate C119

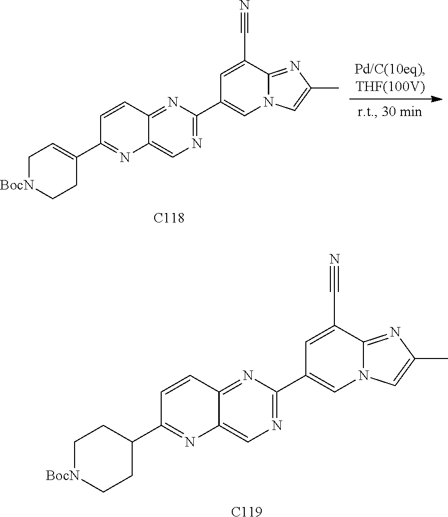 [SMILES: Cc1cn2cc(-c3ncc4nc(C5=CCN(C(=O)OC(C)(C)C)CC5)ccc4n3)cc(C#N)c2n1 ; "C118" ; Cc1cn2cc(-c3ncc4nc(C5CCN(C(=O)OC(C)(C)C)CC5)ccc4n3)cc(C#N)c2n1 ; "C119"]

To a solution of tert-butyl 4-(2-{8-cyano-2-methylimidazo[1,2-a]pyridin-6-yl}pyrido[3,2-d]pyrimidin-6-yl)-3,6-dihydro-2H-pyridine-1-carboxylate (40.0 mg, 0.09 mmol, 1 equiv) in THE (16 mL) was added Pd/C (91.0 mg, 0.86 mmol, 10 equiv) under nitrogen atmosphere in a 50 mL round-bottom flask. The mixture was hydrogenated at room temperature for 30 min under hydrogen atmosphere using a hydrogen balloon, filtered through a Celite pad and concentrated under reduced pressure. The residue was purified by Prep-TLC (PE/EA 1:1) to afford tert-butyl 4-(2-{8-cyano-2-methylimidazo[1,2-a]pyridin-6-yl}pyrido[3,2-d]pyrimidin-6-yl)piperidine-1-carboxylate (17.0 mg, 42%) as a solid. LCMS (ES, m/z): 470 [M+H]⁺

Synthesis of compound 371

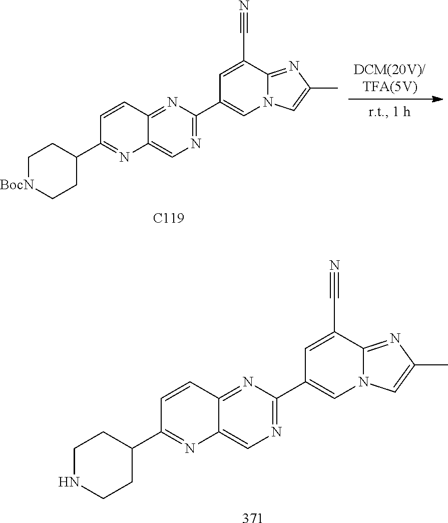 [SMILES: Cc1cn2cc(-c3ncc4nc(C5CCN(C(=O)OC(C)(C)C)CC5)ccc4n3)cc(C#N)c2n1 ; "C119" ; Cc1cn2cc(-c3ncc4nc(C5CCNCC5)ccc4n3)cc(C#N)c2n1 ; "371"]

A solution of tert-butyl 4-(2-{8-cyano-2-methylimidazo[1,2-a]pyridin-6-yl}pyrido[3,2-d]pyrimidin-6-yl)piperidine-1-carboxylate (17.0 mg, 0.03 mmol, 1 equiv) in DCM (2 mL) and TFA (0.5 mL) was stirred for 1 h at room temperature. The resulting mixture was concentrated under vacuum. The crude product (30 mg) was purified by Prep-HPLC (Condition 11, Gradient 1) to afford 2-methyl-6-[6-(piperidin-4-yl)pyrido[3,2-d]pyrimidin-2-yl]imidazo[1,2-a]pyridine-8-carbonitrile (0.9 mg, 7%) as a solid. LCMS (ES, m/z): 370 [M+H]^{+ 1}H NMR (400 MHz, DMSO-d₆) δ 9.89 (d, J=1.6 Hz, 1H), 9.67 (s, 1H), 8.79 (d, J=1.6 Hz, 1H), 8.44 (d, J=8.9 Hz, 1H), 8.10 (s, 1H), 8.03 (d, J=8.9 Hz, 1H), 3.18-3.03 (m, 3H), 2.70 (p, J=1.8 Hz, 1H), 2.65 (d, J=11.3 Hz, 1H), 2.44 (s, 3H), 1.91 (d, J=12.4 Hz, 2H), 1.84-1.69 (m, 2H).

### Example 152: Synthesis of Compounds 366 and 456

### Synthesis of Intermediate C120

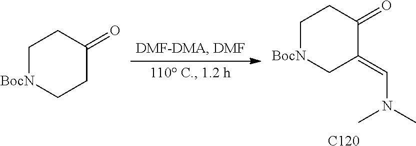 [SMILES: CC(C)(C)OC(=O)N1CCC(=O)CC1 ; CN(C)/C=C1\CN(C(=O)OC(C)(C)C)CCC1=O ; "C120"]

Into a 250 mL round-bottom flask were added tert-butyl 4-oxopiperidine-1-carboxylate (8 g, 40.151 mmol, 1 equiv), DMF-DMA (1.39 g, 60.227 mmol, 1.5 equiv) and DMF (80 mL). The mixture was stirred for 1.2 hr at 110° C., the reaction was quenched by the addition of water (100 mL) at room temperature. The aqueous layer was extracted with ethyl acetate (20 ml×3). The organic layers were washed with brine (20 ml), dried over anhydrous Na₂SO₄. After filtration, the filtrate was concentrated under reduced pressure. This resulted in tert-butyl (3E)-3-[(dimethylamino)methylidene]-4-oxopiperidine-1-carboxylate (10 g, 97%) as an oil.

### Synthesis of Intermediate C121

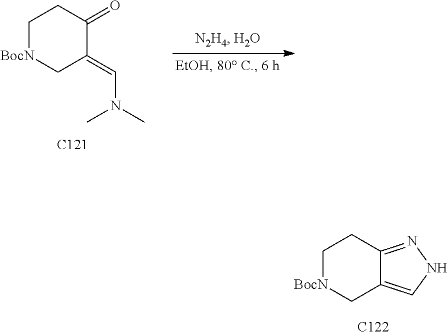 [SMILES: CN(C)/C=C1\CN(C(=O)OC(C)(C)C)CCC1=O ; "C121" ; CC(C)(C)OC(=O)N1CCc2n[nH]cc2C1 ; "C122"]

Into a 250 mL sealed tube were added tert-butyl (3E)-3-[(dimethylamino)methylidene]-4-oxopiperidine-1-carboxylate (9 g, 35.387 mmol, 1 equiv), hydrazine hydrate (1.97 g, 39.319 mmol, 1 equiv) and EtOH (100 mL), the mixture was stirred for 6 hr at 80° C. Then the reaction was quenched by the addition of water (100 mL). The resulting mixture was concentrated under reduced pressure and the aqueous layer was extracted with ethyl acetate (3×25 mL). The combined organic layers were concentrated under reduced pressure. The residue was purified by reverse flash chromatography (Condition 3, Gradient 3) to afford tert-butyl 2H,4H,6H,7H-pyrazolo[4,3-c]pyridine-5-carboxylate (8 g, 91%) as a solid.

### Synthesis of Intermediate C123

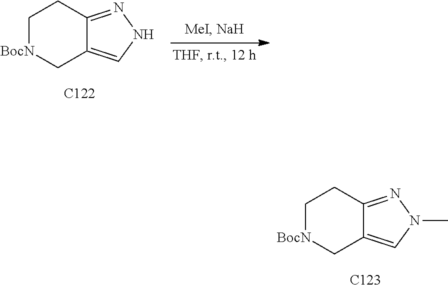 [SMILES: CC(C)(C)OC(=O)N1CCc2n[nH]cc2C1 ; "C122" ; Cn1cc2c(n1)CCN(C(=O)OC(C)(C)C)C2 ; "C123"]

A solution of tert-butyl 2H,4H,6H,7H-pyrazolo[4,3-c]pyridine-5-carboxylate (8 g, 35.830 mmol, 1 equiv) in THF (80 mL) was treated with NaH (1.03 g, 42.996 mmol, 1.2 equiv) for 10 min at 0° C. under nitrogen atmosphere, then added Mel (6.10 g, 42.996 mmol, 1.2 equiv) at room temperature and the mixture was stirred for 12 hr at room temperature. The reaction was quenched by the addition of water (100 mL) at 0° C. The aqueous layer was extracted with ethyl acetate (3×50 mL). The combined organic layers were concentrated under reduced pressure. The crude product was purified by Chiral-Prep-HPLC (Condition 8, Gradient 1) to afford tert-butyl 2-methyl-4H,6H,7H-pyrazolo[4,3-c]pyridine-5-carboxylate (1 g, 12%) as an oil and tert-butyl 1-methyl-4H,6H,7H-pyrazolo[4,3-c]pyridine-5-carboxylate (1.2 g, 14%) as an oil

### Synthesis of Intermediate C124

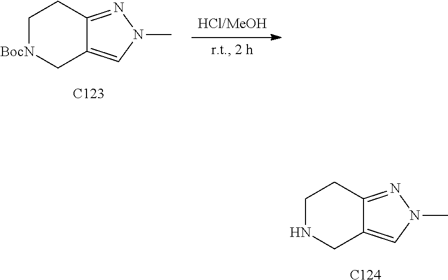

A mixture of tert-butyl 2-methyl-4H,6H,7H-pyrazolo[4,3-c]pyridine-5-carboxylate (1 g, 4.214 mmol, 1 equiv) and HCl(gas) in 1,4-dioxane (10 mL) in MeOH (10 mL) was stirred for 2 hr at room temperature under nitrogen atmosphere. The resulting mixture was concentrated under reduced pressure to afford 2-methyl-4H,5H,6H,7H-pyrazolo[4,3-c]pyridine (450 mg, 77%) as a solid.

### Synthesis of Intermediate C125

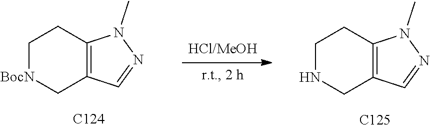

A mixture of tert-butyl 1-methyl-4H,6H,7H-pyrazolo[4,3-c]pyridine-5-carboxylate (1.2 g, 5.057 mmol, 1 equiv) and HCl(gas) in 1,4-dioxane (10 mL) in MeOH (10 mL) was stirred for 2 hr at room temperature under nitrogen atmosphere. The resulting mixture was concentrated under reduced pressure to afford 1-methyl-4H,5H,6H,7H-pyrazolo[4,3-c]pyridine (600 mg, 86%) as a solid.

### Synthesis of Compounds 366 and 456

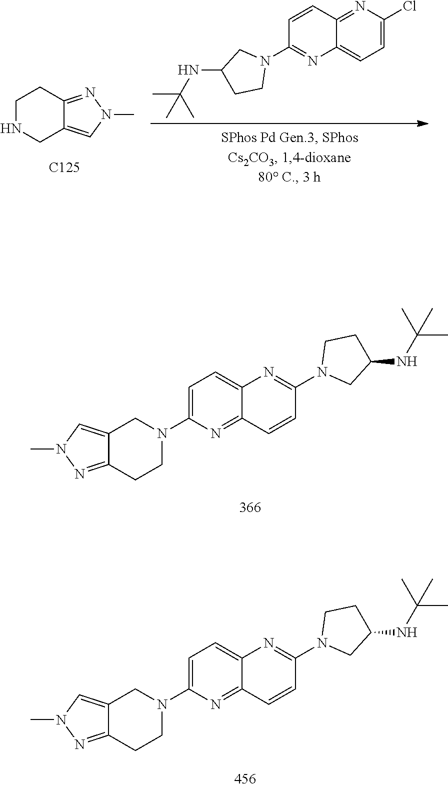

A solution of 2-methyl-4H,5H,6H,7H-pyrazolo[4,3-c]pyridine (45.01 mg, 0.328 mmol, 1 equiv) in 1,4-dioxane (3 mL) was treated with N-tert-butyl-1-(6-chloro-1,5-naphthyridin-2-yl)pyrrolidin-3-amine (100 mg, 0.328 mmol, 1.00 equiv), Cs₂CO₃ (213.78 mg, 0.656 mmol, 2 equiv), SPhos Pd Gen.3 (13.47 mg, 0.033 mmol, 0.1 equiv), SPhos (13.47 mg, 0.033 mmol, 0.1 equiv) for 3 h at 80° C. under nitrogen atmosphere. The resulting mixture was filtered, the filter cake was washed with ethyl acetate (3×10 mL). The filtrate was concentrated under reduced pressure. The crude product was purified by Prep-HPLC (Condition 11, Gradient 2) to afford N-(tert-butyl)-1-(6-(2-methyl-2,4,6,7-tetrahydro-5H-pyrazolo[4,3-c]pyridin-5-yl)-1,5-naphthyridin-2-yl)pyrrolidin-3-amine. The product was then purified by Chiral-Prep-HPLC (Condition 4, Gradient 8) to afford (3R)-N-tert-butyl-1-(6-{2-methyl-4H,6H,7H-pyrazolo[4,3-c]pyridin-5-yl}-1,5-naphthyridin-2-yl)pyrrolidin-3-amine (1.9 mg, 1%) as a solid and (3S)—N-tert-butyl-1-(6-{2-methyl-4H,6H,7H-pyrazolo[4,3-c]pyridin-5-yl}-1,5-naphthyridin-2-yl)pyrrolidin-3-amine (1.6 mg, 1%) as a solid. Compound 366: LCMS (ES, m/z): 406[M+H]+ ¹H NMR (400 MHz, DMSO-d₆) δ 7.77-7.68 (m, 2H), 7.49 (s, 1H), 7.31 (d, J=9.3 Hz, 1H), 6.88 (d, J=9.2 Hz, 1H), 4.62 (s, 2H), 3.92 (t, J=5.9 Hz, 2H), 3.75 (s, 3H), 3.59 (s, 2H), 3.48 (s, 2H), 3.02 (s, 2H), 2.71 (t, J=5.8 Hz, 2H), 2.14 (s, 1H), 1.70 (d, J=9.7 Hz, 1H), 1.08 (s, 9H). Compound 456: LCMS (ES, m/z): 406[M+H]+ ¹H NMR-(400 MHz, DMSO-d₆) δ 7.73 (dd, J=9.1, 6.3 Hz, 2H), 7.49 (s, 1H), 7.31 (d, J=9.4 Hz, 1H), 6.88 (d, J=9.2 Hz, 1H), 4.62 (s, 2H), 3.92 (t, J=5.8 Hz, 2H), 3.75 (s, 3H), 3.59 (t, J=8.2 Hz, 2H), 3.49 (s, 2H), 3.03 (s, 1H), 2.71 (t, J=5.8 Hz, 1H), 2.14 (s, 2H), 1.72 (s, 1H), 1.24 (s, 1H), 1.08 (s, 9H).

### Example 153: Synthesis of Compounds 401 and 402

### Synthesis of Intermediate C126

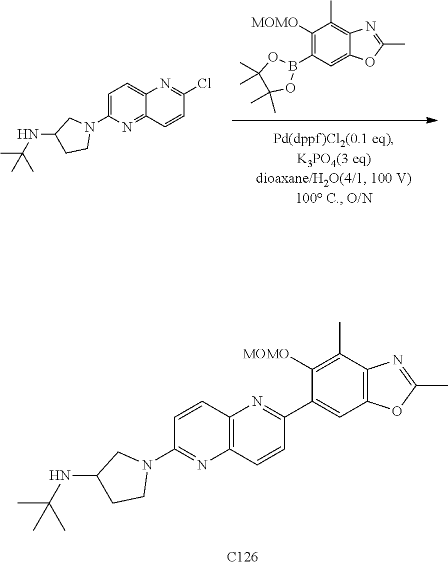

A mixture of compound N-tert-butyl-1-(6-chloro-1,5-naphthyridin-2-yl)pyrrolidin-3-amine (4.7 g, 15.419 mmol, 1 equiv) and Pd(dppf)Cl₂ (1.13 g, 1.542 mmol, 0.1 equiv) was add to dioxane (400 mL) taken in a round bottom flask. Then, K₃PO₄ (9.82 g, 46.257 mmol, 3 equiv) in H₂O (100 mL) was added to mixture. After stirring for 5 min at room temperature, 5-(methoxymethoxy)-2,4-dimethyl-6-(4,4,5,5-tetramethyl-1,3,2-dioxaborolan-2-yl)-1,3-benzoxazole (6.16 g, 18.503 mmol, 1.2 equiv) was added in portions. The resulting mixture was stirred at 100° C. overnight under a nitrogen atmosphere. The mixture was allowed to cool down to room temperature. The resulting mixture was diluted with water (60 mL). The resulting mixture was extracted with ethyl acetate (3×60 mL). The combined organic layers were washed with brine (1×50 mL), dried over anhydrous Na₂SO₄. After filtration, the filtrate was concentrated under reduced pressure. The residue was purified by silica gel column chromatography, eluted with PE/EA (1:1) to afford N-tert-butyl-1-{6-[5-(methoxymethoxy)-2,4-dimethyl-1,3-benzoxazol-6-yl]-1,5-naphthyridin-2-yl}pyrrolidin-3-amine (4.4 g, 56%) as a solid. LCMS (ESI, m/z): 476[M+H]⁺.

### Synthesis of Intermediate C127

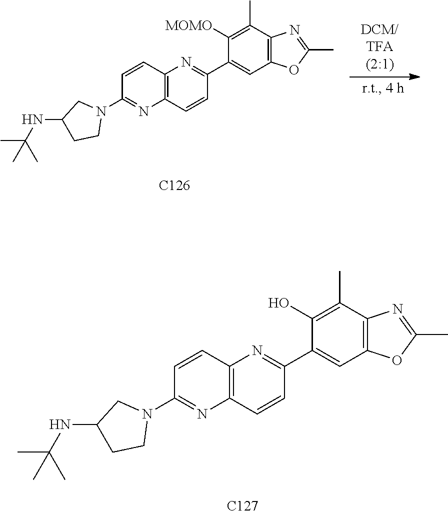

To a solution of N-tert-butyl-1-{6-[5-(methoxymethoxy)-2,4-dimethyl-1,3-benzoxazol-6-yl]-1,5-naphthyridin-2-yl}pyrrolidin-3-amine (2 g, 4.205 mmol, 1 equiv) in DCM (10 mL, 157.306 mmol, 37.41 equiv) were added TFA (5 mL, 67.315 mmol, 16.01 equiv). After stirring for 4 h at room temperature under a nitrogen atmosphere, the resulting mixture was concentrated under reduced pressure. The crude product was obtained 6-{6-[3-(tert-butylamino)pyrrolidin-1-yl]-1,5-naphthyridin-2-yl}-2,4-dimethyl-1,3-benzoxazol-5-ol (1.6 g, 88%) as a solid. LCMS (ESI, m/z): 432[M+H]⁺.

### Synthesis of Compounds 401 and 402

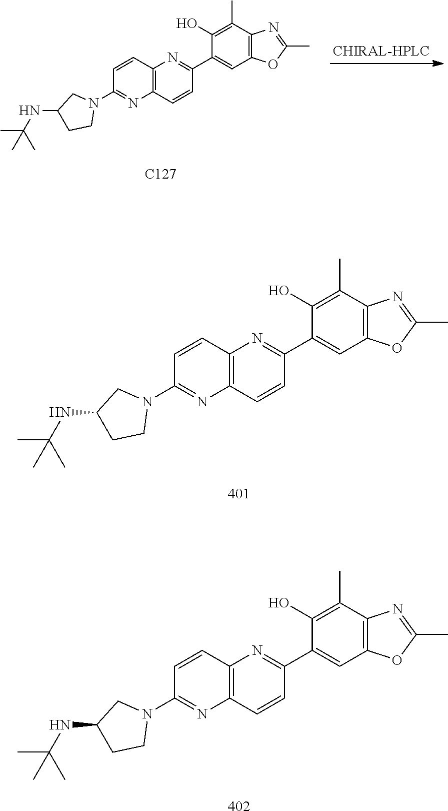

6-{6-[3-(tert-butylamino)pyrrolidin-1-yl]-1,5-naphthyridin-2-yl}-2,4-dimethyl-1,3-benzoxazol-5-ol (1.6 g, 3.711 mmol, 1 equiv) was purified by chiral-prep-HPLC (Condition 5, Gradient 1) to afford 6-{6-[(3R)-3-(tert-butylamino)pyrrolidin-1-yl]-1,5-naphthyridin-2-yl}-2,4-dimethyl-1,3-benzoxazol-5-ol (517.2 mg, 27%) and 6-{6-[(3S)-3-(tert-butylamino)pyrrolidin-1-yl]-1,5-naphthyridin-2-yl}-2,4-dimethyl-1,3-benzoxazol-5-ol (480.3 mg, 26%) as a solid.

### Analysis Data

| Compound Structure | Analysis data |
|---|---|
| 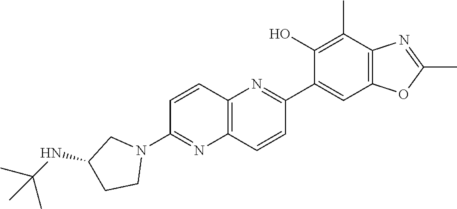 [SMILES: Cc1nc2c(C)c(O)c(-c3ccc4nc(N5CC[C@H](NC(C)(C)C)C5)ccc4n3)cc2o1] | LCMS (ES, m/z): 432 [M + H] ⁺ ¹H NMR (400 MHz, DMSO-d₆) δ 14.99 (s, 1H), 8.41 (d, J = 9.4 Hz, 1H), 8.25 (s, 1H), 8.14 (d, J = 9.3 Hz, 1H), 8.08 (d, J = 9.3 Hz, 1H), 7.16 (d, J = 9.3 Hz, 1H), 3.92-3.82 (m, 1H), 3.77-3.65 (m, 1H), 3.57-3.45 (m, 2H), 3.17-3.09 (m, 1H), 2.61 (s, 3H), 2.40 (s, 3H), 2.23-2.16 (m, 1H), 1.80-1.70 (m, 1H), 1.10 (s, 9H). |
| 402 | |
| 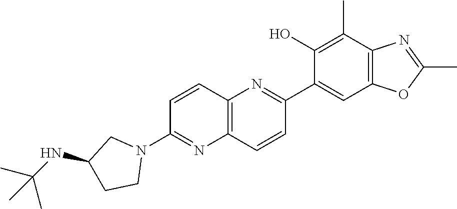 [SMILES: Cc1nc2c(C)c(O)c(-c3ccc4nc(N5CC[C@@H](NC(C)(C)C)C5)ccc4n3)cc2o1] | LCMS (ES, m/z): 432 [M + H] ⁺ ¹H NMR (400 MHz, DMSO-d₆) δ 14.99 (s, 1H), 8.42 (d, J = 9.4 Hz, 1H), 8.26 (s, 1H), 8.15 (d, J = 9.3 Hz, 1H), 8.08 (d, J = 9.1 Hz, 1H), 7.17 (d, J = 9.3 Hz, 1H), 3.91-3.84 (m, 1H), 3.76-3.70 (m, 1H), 3.56-3.43 (m, 2H), 3.16-3.09 (m, 1H), 2.61 (s, 3H), 2.40 (s, 3H), 2.33 (dt, J = 3.6, 1.8 Hz, 1H), 1.83-1.74 (m, 1H), 1.11 (s, 9H). |
| 401 | |

### Example 154: Synthesis of Compounds 350, 351, and 374-377

### Synthesis of Intermediate C128

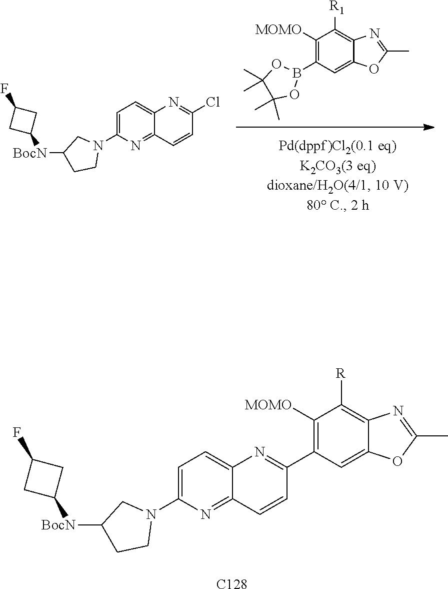

A solution of tert-butyl N-[1-(6-chloro-1,5-naphthyridin-2-yl)pyrrolidin-3-yl]-N-[(1s,3s)-3-fluorocyclobutyl]carbamate (350 mg, 0.832 mmol, 1 equiv) and 5-(methoxymethoxy)-2-methyl-6-(4,4,5,5-tetramethyl-1,3,2-dioxaborolan-2-yl)-1,3-benzoxazole (796 mg, 2.496 mmol, 3 equiv), Pd(dppf)Cl₂ (68 mg, 0.083 mmol, 0.1 equiv), K₂CO₃ (345 mg, 2.496 mmol, 3 equiv) in 1,4-dioxane (2.8 mL), water (0.7 mL) was stirred for 2 hr at 80° C. under nitrogen atmosphere. The resulting mixture was extracted with ethyl acetate (1×30 mL). dried over anhydrous Na₂SO₄. After filtration, the filtrate was concentrated under reduced pressure. The residue was purified by silica gel column chromatography, eluted with PE/EA (1:1) to afford tert-butyl N-(1-{6-[5-(methoxymethoxy)-2-methyl-1,3-benzoxazol-6-yl]-1,5-naphthyridin-2-yl}pyrrolidin-3-yl)-N-[(1s,3s)-3-fluorocyclobutyl]carbamate (175 mg, 36%) as a solid.

### Synthesis of Intermediate C129

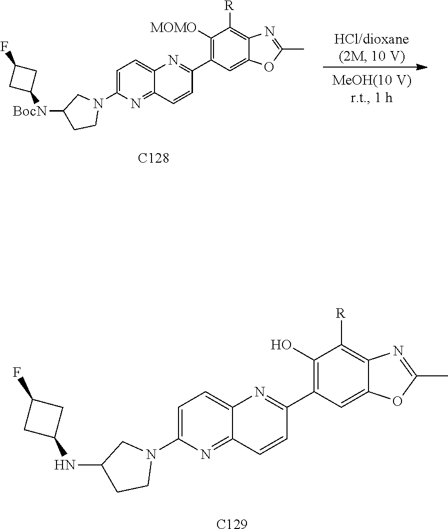

A solution of tert-butyl N-(1-{6-[5-(methoxymethoxy)-2-methyl-1,3-benzoxazol-6-yl]-1,5-naphthyridin-2-yl}pyrrolidin-3-yl)-N-[(1s,3s)-3-fluorocyclobutyl]carbamate (173 mg, 0.299 mmol, 1 equiv) in MeOH (1.7 mL), HCl(gas) in 1,4-dioxane (1.7 mL) was stirred for 1 h at room temperature. The resulting mixture was concentrated under reduced pressure. The residue was purified by trituration with MeOH/DCM=10/1(2.75 mL). The resulting mixture was filtered, the filter cake was washed with MeOH (1×2 mL). The filtrate was concentrated under reduced pressure. This resulted in 2-methyl-6-[6-(3-{[(1s,3s)-3-fluorocyclobutyl]amino}pyrrolidin-1-yl)-1,5-naphthyridin-2-yl]-1,3-benzoxazol-5-ol (91 mg, 70%) as a solid.

### Synthesis of Intermediate C130

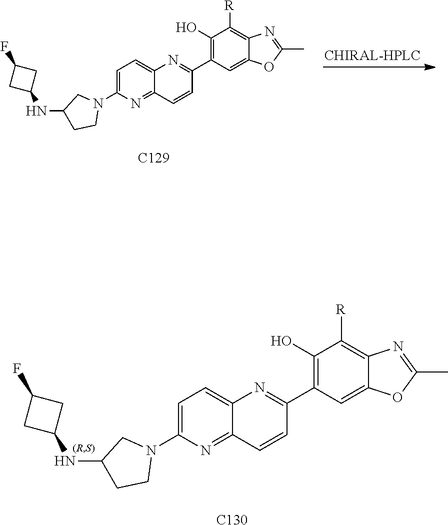

The 2-methyl-6-[6-(3-{[(1s,3s)-3-fluorocyclobutyl]amino}pyrrolidin-1-yl)-1,5-naphthyridin-2-yl]-1,3-benzoxazol-5-ol (91 mg) was purified by Chiral-HPLC with the following conditions (Column: CHIRAL ART Cellulose-SB, 2×25 cm, 5 μm; Mobile Phase A: MtBE(0.1% DEA)-HPLC, Mobile Phase B: MeOH-HPLC; Flow rate: 20 mL/min; Gradient: 20% B to 20% B in 8.3 min; Wave Length: UV 254/220 nm; RT1(min): 6.09; RT2(min): 7.28; Sample Solvent: MeOH: DCM=1:1-HPLC; Injection Volume: 0.5 mL; Number Of Runs: 30) to afford (38.4 mg, 37.25%) as a solid and (38.5 mg, 37.73%) as a solid.

### Analysis Data

| Compound Structure | Analysis data |
|---|---|
| 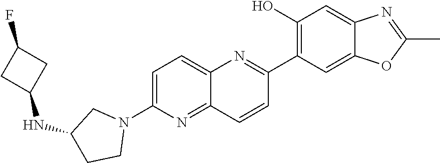 [SMILES: Cc1nc2cc(O)c(-c3ccc4nc(N5CC[C@H](N[C@H]6C[C@@H](F)C6)C5)ccc4n3)cc2o1] | LCMS (ES, m/z): 434 [M + H] ⁺ ¹H NMR (400 MHz, DMSO-d₆) δ 14.52 (s, 1H), 8.46-8.39 (m, 2H), 8.16-8.05 (m, 2H), 7.19-7.09 (m, 2H), 4.85 (p, J = 7.0 Hz, 1H), 3.69 (s, 2H), 3.56 (s, 1H), 3.39 (s, 2H), 2.80 (s, 1H), 2.71-2.63 (m, 2H), 2.45 (s, 3H), 1.94 (s, 1H), 1.86 (s, 3H). |
| 396 | |
| 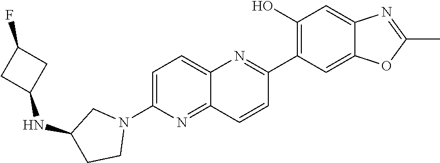 [SMILES: Cc1nc2cc(O)c(-c3ccc4nc(N5CC[C@@H](N[C@H]6C[C@@H](F)C6)C5)ccc4n3)cc2o1] | LCMS (ES, m/z): 434 [M + H] ⁺ ¹H NMR (400 MHz, DMSO-d₆) δ 14.51 (s, 1H), 8.46-8.39 (m, 2H), 8.16-8.05 (m, 2H), 7.16-7.12 (s, 2H), 4.86-4.72 (p, J = 7.0 Hz, 1H), 3.70 (s, 2H), 3.56 (d, J = 9.7 Hz, 1H), 3.38 (d, J = 9.7 Hz, 2H), 2.80 (s, 1H), 2.67 (d, J = 17.5 Hz, 2H), 2.61 (s, 3H), 2.16-2.05 (m, 1H), 1.94-1.86 (s, 3H). |
| 395 | |
| 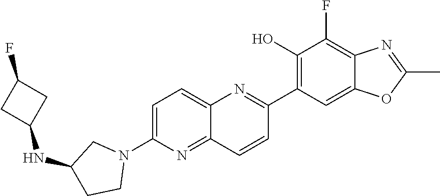 [SMILES: Cc1nc2c(F)c(O)c(-c3ccc4nc(N5CC[C@@H](N[C@H]6C[C@@H](F)C6)C5)ccc4n3)cc2o1] | LCMS (ES, m/z): 452 [M + H] + ¹H NMR (400 MHz, DMSO-d₆) δ 15.06 (s, 1H), 8.44 (d, J = 9.2 Hz, 1H), 8.32 (d, J = 1.4 Hz, 1H), 8.13 (ddd, J = 22.4, 9.2, 0.8 Hz, 2H), 7.18 (d, J = 9.3 Hz, 1H), 4.78 (dp, J = 56.4, 7.0 Hz, 1H), 3.70 (d, J = 6.1 Hz, 2H), 3.56 (d, J = 9.1 Hz, 1H), 3.37 (d, J = 5.5 Hz, 1H), 2.84-2.73 (m, 1H), 2.70-2.61 (m, 5H), 2.33 (p, J = 1.9 Hz, 1H), 2.10 (dq, J = 12.6, 6.4 Hz, 1H), 1.99-1.76 (m, 3H). |
| 375 | |
| 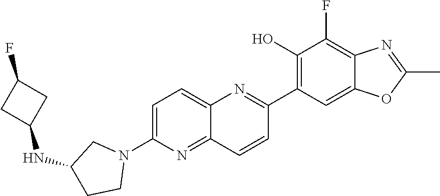 [SMILES: Cc1nc2c(F)c(O)c(-c3ccc4nc(N5CC[C@H](N[C@H]6C[C@@H](F)C6)C5)ccc4n3)cc2o1] | LCMS (ES, m/z): 452 [M + H] +; ¹H NMR (400 MHz, DMSO-d₆) δ 15.06 (s, 1H), 8.44 (d, J = 9.2 Hz, 1H), 8.32 (d, J = 1.4 Hz, 1H), 8.13 (ddd, J = 22.4, 9.2, 0.8 Hz, 2H), 7.18 (d, J = 9.3 Hz, 1H), 4.78 (dp, J = 56.4, 7.0 Hz, 1H), 3.70 (d, J = 6.1 Hz, 2H), 3.56 (d, J = 9.1 Hz, 1H), 3.37 (d, J = 5.5 Hz, 1H), 2.84-2.73 (m, 1H), 2.70-2.71(m, 1H), 2.70-2.61 (m, 4H), 2.33 (p, J = 1.9 Hz, 1H), 2.10 (dq, J = 12.6, 6.4 Hz, 1H), 1.99-1.76 (m, 3H). |
| 374 | |
| 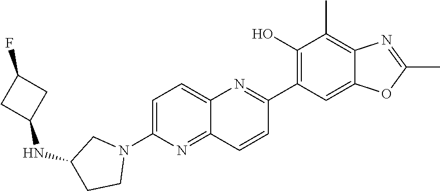 [SMILES: Cc1nc2c(C)c(O)c(-c3ccc4nc(N5CC[C@H](N[C@H]6C[C@@H](F)C6)C5)ccc4n3)cc2o1] | LCMS (ES, m/z): 448 [M + H] ⁺ ¹H NMR (400 MHz, DMSO-d₆) δ 14.98 (s, 1H), 8.41 (d, J = 9.3 Hz, 1H), 8.25 (s, 1H), 8.14 (dd, J = 9.2, 0.8 Hz, 1H), 8.07 (dd, J = 9.1, 0.8 Hz, 1H), 7.16 (d, J = 9.3 Hz, 1H), 4.78 (dp, J = 56.3, 7.0 Hz, 1H), 3.77- 3.66 (m, 2H), 3.55 (d, J = 9.1 Hz, 1H), 3.37 (d, J = 5.5 Hz, 1H), 3.29 (s, 1H), 2.77 (s, 1H), 2.65 (dt, J = 11.8, 5.8 Hz, 2H), 2.61 (s, 3H), 2.40 (s, 3H), 2.30 (s, 1H), 2.10 (dq, J = 12.2, 5.8, 5.3 Hz, 1H), 1.98- 1.78 (m, 3H). |
| 377 | |
| 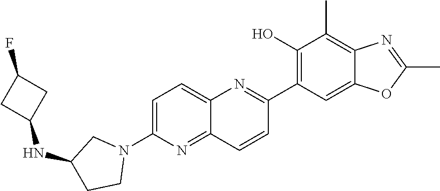 [SMILES: Cc1nc2c(C)c(O)c(-c3ccc4nc(N5CC[C@@H](N[C@H]6C[C@@H](F)C6)C5)ccc4n3)cc2o1] | LCMS (ES, m/z): 448 [M + H] + ¹H NMR (400 MHz, DMSO-d₆) δ 14.98 (s, 1H), 8.41 (d, J = 9.3 Hz, 1H), 8.25 (s, 1H), 8.15-8.12 (m, 1H), 8.07 (dd, J = 9.1, 0.8 Hz, 1H), 7.15 (d, J = 9.3 Hz, 1H), 4.78 (dp, J = 56.4, 7.0 Hz, 1H), 3.75-3.66 (m, 2H), 3.55 (d, J = 9.3 Hz, 1H), 3.37 (d, J = 5.4 Hz, 1H), 3.28 (s, 1H), 2.78 (s, 1H), 2.72-2.60 (m, 2H), 2.61 (s, 3H), 2.40 (s, 3H), 2.30 (s, 1H), 2.10 (dq, J = 12.6, 5.8, 5.4 Hz, 1H), 1.99-1.78 (m, 3H). |
| 376 | |

### Example 155: Synthesis of Compound 448

### Synthesis of Intermediate C130

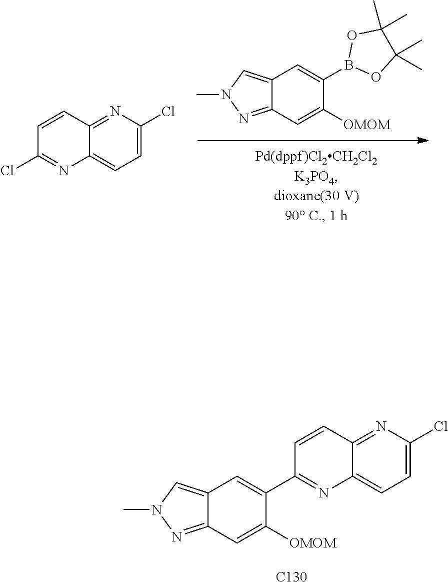

Into a 40 mL vial were added 2,6-dichloro-1,5-naphthyridine (500 mg, 2.512 mmol, 1 equiv), dioxane (10 mL), K₃PO₄ (1599.75 mg, 7.536 mmol, 3 equiv) in H₂O (2 mL) and Pd(dppf)Cl₂·CH₂Cl₂ (204.65 mg, 0.251 mmol, 0.1 equiv) at room temperature. The resulting mixture was stirred for 1 h at 90° C. under nitrogen atmosphere. The mixture was allowed to cool down to room temperature. The reaction was quenched by the addition of Water (30 mL) at room temperature. The resulting mixture was extracted with EtOAc (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:5) to afford 2-chloro-6-[6-(methoxymethoxy)-2-methylindazol-5-yl]-1,5-naphthyridine (220 mg, 24.68%) as a light yellow solid. LCMS (ES, m/z):355 [M+H]⁺

### Synthesis of Intermediate C131

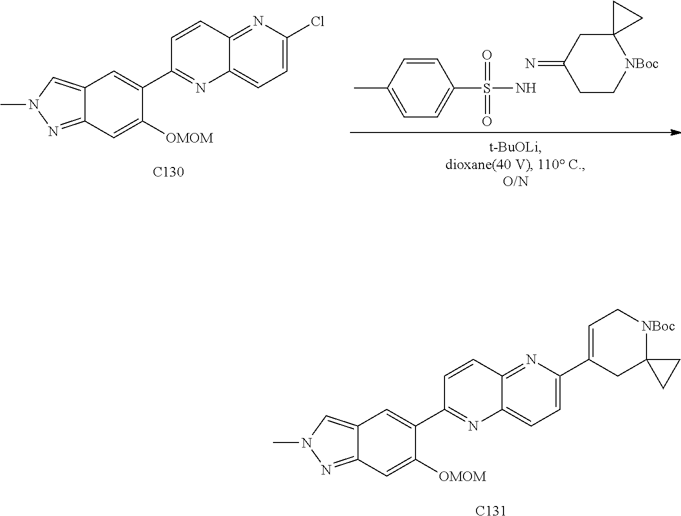

Into a 40 mL vial were added 2-chloro-6-[6-(methoxymethoxy)-2-methylindazol-5-yl]-1,5-naphthyridine (180 mg, 0.507 mmol, 1 equiv), tert-butyl (7E)-7-[(4-methylbenzenesulfonamido)imino]-4-azaspiro [2.5]octane-4-carboxylate (199.64 mg, 0.507 mmol, 1 equiv), t-BuOLi (121.85 mg, 1.521 mmol, 3 equiv), dioxane (20 mL, 236.079 mmol, 465.33 equiv) and Pd(dppf)Cl₂CH₂Cl₂ (41.33 mg, 0.051 mmol, 0.1 equiv) at room temperature. The resulting mixture was stirred overnight at 110° C. under nitrogen atmosphere. The mixture was allowed to cool down to room temperature. The reaction was quenched by the addition of water (40 mL) at room temperature. The resulting mixture was extracted with CH₂Cl₂ (3×30 mL). The combined organic layers were washed with brine (1×30 mL), dried over anhydrous Na₂SO₄. After filtration, the filtrate was concentrated under reduced pressure. The residue was purified by silica gel column chromatography, eluted with PE/EA (1:4) to afford tert-butyl 7-{6-[6-(methoxymethoxy)-2-methylindazol-5-yl]-1,5-naphthyridin-2-yl}-4-azaspiro[2.5]oct-6-ene-4-carboxylate (70 mg, 26%) as a solid. LCMS (ES, m/z):528 [M+H]⁺

### Synthesis of Intermediate C132

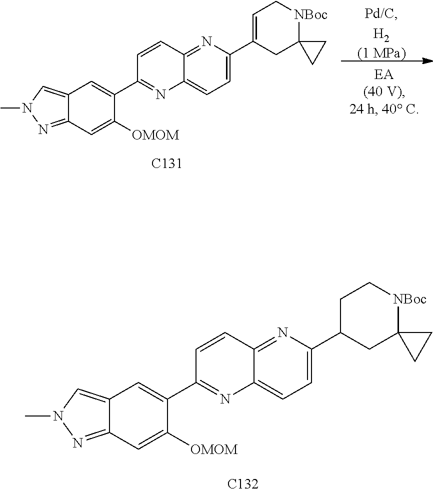

Into a 25 mL round-bottom flask were added tert-butyl 7-{6-[6-(methoxymethoxy)-2-methylindazol-5-yl]-1,5-naphthyridin-2-yl}-4-azaspiro[2.5]oct-6-ene-4-carboxylate (63 mg, 0.119 mmol, 1 equiv), Pd/C (2.54 mg, 0.024 mmol, 0.2 equiv), EA (3 mL) at room temperature. The resulting mixture was stirred for 24 hr at 40° C. under hydrogen atmosphere (1 MPa). The resulting mixture was filtered, the filter cake was washed with MeOH (3×3 mL). The filtrate was concentrated under reduced pressure. The crude product was used in the next step directly without further purification. LCMS (ES, m/z):530 [M+H]⁺

### Synthesis of Compound 448

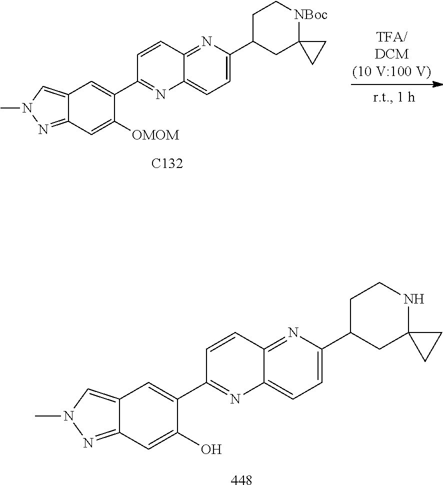

Into a 8 mL vial were added tert-butyl 7-{6-[6-(methoxymethoxy)-2-methylindazol-5-yl]-1,5-naphthyridin-2-yl}-4-azaspiro[2.5]octane-4-carboxylate (40 mg, 0.076 mmol, 1 equiv), DCM (2 mL) and TFA (0.2 mL) at room temperature. The resulting mixture was stirred for 1 h at room temperature. The mixture was basified to pH 8 with NH₃/MeOH. The resulting mixture was concentrated under reduced pressure. The crude product (42 mg) was purified by Prep-(condition 11, Gradient 1) to afford 5-(6-{4-azaspiro[2.5]octan-7-yl}-1,5-naphthyridin-2-yl)-2-methylindazol-6-ol (7.2 mg, 19.82%) as a solid. LCMS (ES, m/z):386 [M+H]+ ¹H NMR (400 MHz, DMSO-d₆) δ 13.62 (s, 1H), 8.69 (s, 1H), 8.60 (d, J=9.2 Hz, 1H), 8.47 (d, J=9.1 Hz, 1H), 8.41 (d, J=8.1 Hz, 2H), 7.78 (d, J=8.7 Hz, 1H), 6.92 (s, 1H), 4.13 (s, 3H), 3.28-3.16 (m, 1H), 3.01 (d, J=12.7 Hz, 1H), 2.79-2.68 (m, 1H), 2.18 (t, J=12.4 Hz, 1H), 2.04 (s, 1H), 1.91 (d, J=12.8 Hz, 1H), 1.74 (td, J=12.4, 4.1 Hz, 1H), 1.33-1.25 (m, 1H), 0.59-0.50 (m, 1H), 0.48-0.35 (m, 3H).

### Example 156: Synthesis of Compounds 362 and 363

### Synthesis of Intermediate C133

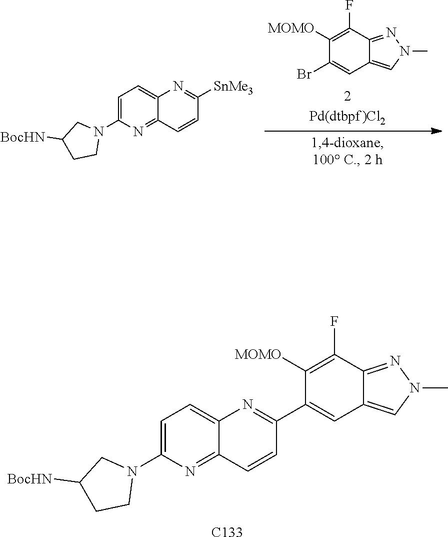

Into a 40 mL round-bottom flask were added tert-butyl N-{1-[6-(trimethylstannyl)-1,5-naphthyridin-2-yl]pyrrolidin-3-yl}carbamate (550 mg, 1.153 mmol, 1 equiv), 5-bromo-7-fluoro-6-(methoxymethoxy)-2-methylindazole (333.21 mg, 1.153 mmol, 1 equiv), Pd(dtbpf)Cl₂ (75.12 mg, 0.115 mmol, 0.1 equiv) and 1,4-dioxane (10 mL). The mixture solution was stirred for 2 hr at 100° C. The resulting mixture was concentrated under vacuum. This resulted in tert-butyl N-(1-{6-[7-fluoro-6-(methoxymethoxy)-2-methylindazol-5-yl]-1,5-naphthyridin-2-yl}pyrrolidin-3-yl)carbamate (380 mg, 63%) as a solid.

### Synthesis of Compounds 362 and 363

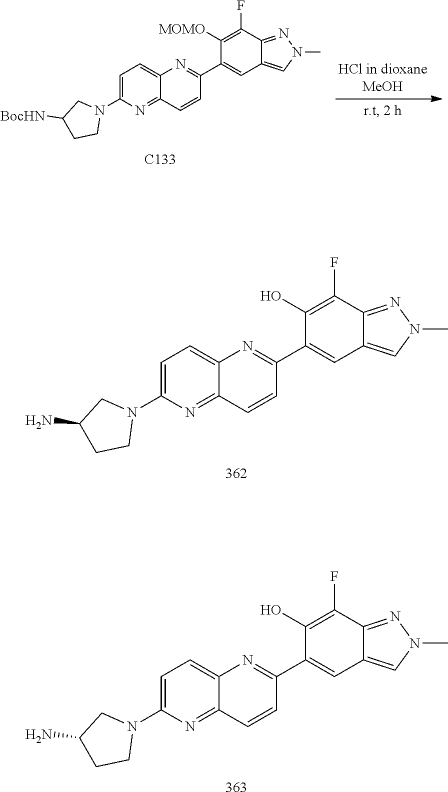

A mixture of tert-butyl N-(1-{6-[7-fluoro-6-(methoxymethoxy)-2-methylindazol-5-yl]-1,5-naphthyridin-2-yl}pyrrolidin-3-yl)carbamate (350 mg, 0.670 mmol, 1 equiv) and HCl(gas) in 1,4-dioxane (3 mL) in methanol (3 mL) was stirred for 2 hr at room temperature under nitrogen atmosphere. The resulting mixture was concentrated under reduced pressure. The crude product was purified by Chiral-Prep-HPLC (Condition 6, Gradient 2) to afford 5-{6-[(3R)-3-aminopyrrolidin-1-yl]-1,5-naphthyridin-2-yl}-7-fluoro-2-methylindazol-6-ol (5.7 mg, 2%) as a solid and 5-{6-[(3R)-3-aminopyrrolidin-1-yl]-1,5-naphthyridin-2-yl}-7-fluoro-2-methylindazol-6-ol (4.5 mg, 2%) as a solid. LCMS (ES, m/z): 379 [M+H]+ ¹H NMR (400 MHz, DMSO-d₆) δ 14.34 (s, 1H), 8.49 (d, J=2.6 Hz, 1H), 8.45 (d, J=9.3 Hz, 1H), 8.43 (d, J=1.0 Hz, 1H), 8.23 (d, J=9.3 Hz, 1H), 8.14 (d, J=9.4 Hz, 3H), 7.25 (d, J=9.3 Hz, 1H), 4.16 (s, 3H), 4.00 (d, J=3.7 Hz, 1H), 3.87 (dd, J=12.1, 6.1 Hz, 1H), 3.83-3.62 (m, 3H), 2.40 (dt, J=13.7, 7.1 Hz, 1H), 2.21-2.12 (m, 1H). LCMS (ES, m/z): 379 [M+H]+ ¹H NMR (400 MHz, DMSO-d₆) δ 14.34 (s, 1H), 8.49 (d, J=2.7 Hz, 1H), 8.45 (d, J=9.3 Hz, 1H), 8.43 (d, J=1.0 Hz, 1H), 8.23 (dd, J=9.2, 0.8 Hz, 1H), 8.20-8.11 (m, 3H), 7.25 (d, J=9.3 Hz, 1H), 4.16 (s, 3H), 4.03-3.97 (m, 1H), 3.87 (dd, J=12.1, 6.0 Hz, 1H), 3.80-3.68 (m, 3H), 2.39 (dd, J=13.7, 6.4 Hz, 1H), 2.21-2.12 (m, 1H).

### Example 157: Synthesis of Compounds 406 and 407

### Synthesis of Intermediate C134

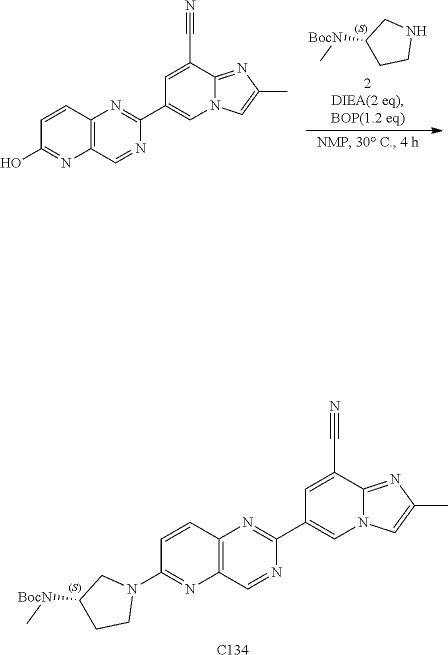

A mixture of compound 6-{6-hydroxypyrido[3,2-d]pyrimidin-2-yl}-2-methylimidazo[1,2-a]pyridine-8-carbonitrile (40 mg, 0.132 mmol, 1 equiv) and tert-butyl N-methyl-N-[(3S)-pyrrolidin-3-yl]carbamate (31.80 mg, 0.158 mmol, 1.2 equiv) in NMP (7.5 mL, 77.774 mmol, 587.77 equiv) was taken in a 8 ml vial. Then DIEA (34.20 mg, 0.264 mmol, 2 equiv) and BOP (70.23 mg, 0.158 mmol, 1.2 equiv) was added to mixture refluxed with stirring for 4 h at 30° C. After the reaction was finish, the mixture was allowed to cool down to room temperature. The resulting mixture was diluted with water (10 mL). The resulting mixture was extracted with ethyl acetate (3×10 mL). The combined organic layers were washed with brine (1×10 mL), dried over anhydrous Na₂SO₄. After filtration, the filtrate was concentrated under reduced pressure. The residue was purified by silica gel column chromatography, eluted with DCM/MeOH (1:10) to afford tert-butyl N-[(3S)-1-(2-{8-cyano-2-methylimidazo[1,2-a]pyridin-6-yl}pyrido[3,2-d]pyrimidin-6-yl)pyrrolidin-3-yl]-N-methylcarbamate (30 mg, 46%). LCMS (ESI, m/z): 485[M+H]⁺.

### Synthesis of Compound 406

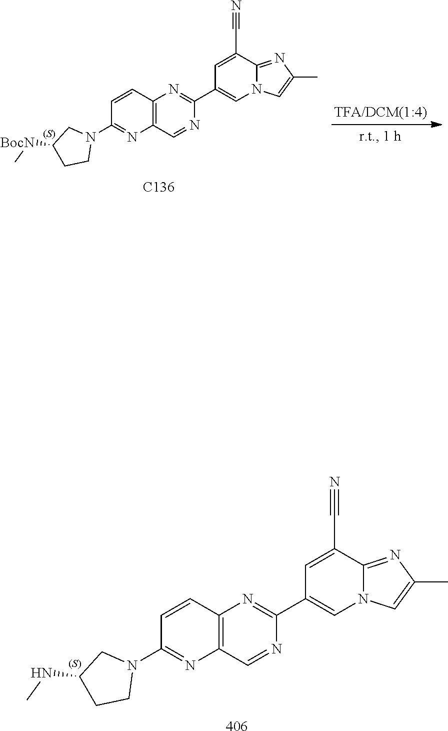

To a solution of tert-butyl N-[(3S)-1-(2-{8-cyano-2-methylimidazo[1,2-a]pyridin-6-yl}pyrido[3,2-d]pyrimidin-6-yl)pyrrolidin-3-yl]-N-methylcarbamate (30 mg, 0.062 mmol, 1 equiv) in DCM (4 mL) were added TFA (1 mL). After stirring for 1 hr at room temperature under a nitrogen atmosphere, the resulting mixture was concentrated under reduced pressure. The crude product was purified by Prep-HPLC (Condition 12, Gradient 3) to afford 2-methyl-6-{6-[(3S)-3-(methylamino)pyrrolidin-1-yl]pyrido[3,2-d]pyrimidin-2-yl}imidazo[1,2-a]pyridine-8-carbonitrile (3.2 mg, 13%) as a solid. LCMS (ESI, m/z): 385 [M+H]⁺. ¹H NMR (400 MHz, DMSO-d₆) δ 9.78 (s, 1H), 9.20 (s, 1H), 8.69 (s, 1H), 8.09 (d, J=9.6 Hz, 2H), 7.41 (d, J=10.0 Hz, 1H), 3.76-3.61 (m, 5H), 2.42 (s, 3H), 2.33 (s, 3H), 2.16-2.10 (m, 1H), 1.89 (d, J=6.5 Hz, 1H).

### Synthesis of Intermediate C137

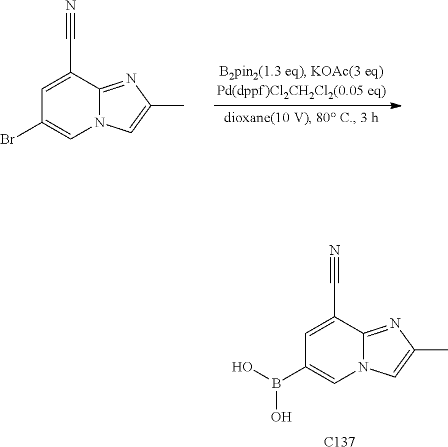

Into a 40 mL sample bottle were added 6-bromo-2-methylimidazo[1,2-a]pyridine-8-carbonitrile (500 mg, 2.11 mmol, 1 equiv), bis(pinacolato)diboron (699.20 mg, 2.75 mmol, 1.3 equiv), KOAc (623.59 mg, 6.35 mmol, 3 equiv), Pd(dppf)Cl₂CH₂Cl₂ (86.27 mg, 0.10 mmol, 0.05 equiv) and dioxane (10 mL). The resulting mixture was stirred for 3 hr at 80° C. under nitrogen atmosphere. The solids were filtered out. The crude product was used in the next step directly without further purification. LCMS: 202[M+H]⁺

### Synthesis of Intermediate C138

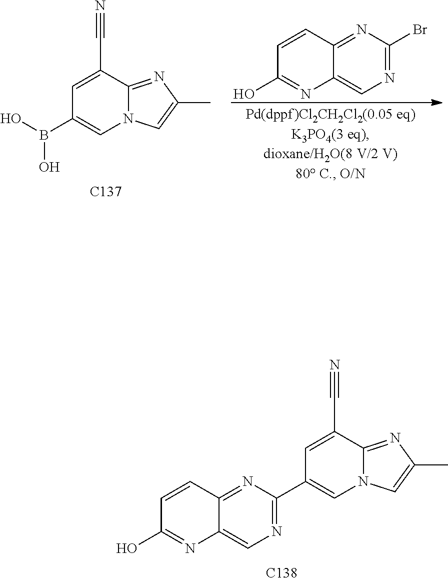

Into two 40 mL sample bottles were added 2-bromopyrido[3,2-d]pyrimidin-6-ol (400 mg, 1.77 mmol, 1.00 equiv), K₃PO₄ (1126.91 mg, 5.31 mmol, 3 equiv) in H₂O (8 mL) and Pd(dppf)Cl₂CH₂Cl₂ (115.33 mg, 0.142 mmol, 0.08 equiv). To the above mixture was dropwise 8-cyano-2-methylimidazo[1,2-a]pyridin-6-ylboronic acid (355.68 mg, 1.77 mmol, 1 equiv) in dioxane (40 mL). The resulting mixture was stirred for overnight at 80° C. under nitrogen atmosphere. The mixture was allowed to cool down to room temperature. The reaction was quenched by the addition of water (20 mL). The mixture was filtered out, the filter was concentrated and washed MTBE. After filtration, the precipitated solids were collected. This resulted in 6-{6-hydroxypyrido[3,2-d]pyrimidin-2-yl}-2-methylimidazo[1,2-a]pyridine-8-carbonitrile (200 mg, crude 37%) as a solid. LCMS: 303[M+H]⁺

### Synthesis of Intermediate C139

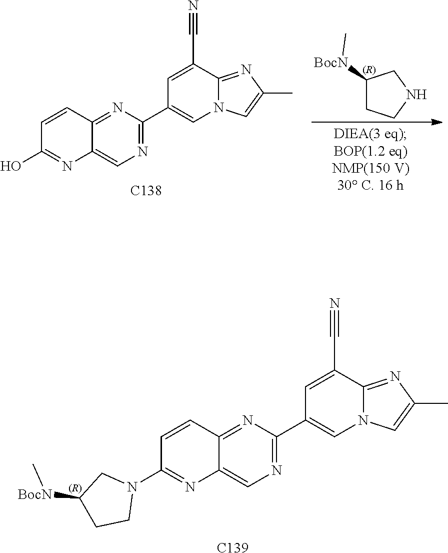

Into a 40 mL sample bottle were added 6-{6-hydroxypyrido[3,2-d]pyrimidin-2-yl}-2-methylimidazo[1,2-a]pyridine-8-carbonitrile (50 mg, 0.16 mmol, 1 equiv), tert-butyl N-methyl-N-[(3R)-pyrrolidin-3-yl]carbamate (39.75 mg, 0.19 mmol, 1.2 equiv), DIEA (64.13 mg, 0.49 mmol, 3 equiv), BOP (87.79 mg, 0.19 mmol, 1.2 equiv) and NMP (15 mL). The resulting mixture was stirred for overnight at 30° C. The reaction was quenched with Water (30 mL). The resulting mixture was extracted with ethyl acetate (3×30 mL). The combined organic layers were washed with NaCl (30 mL) dried over anhydrous Na₂SO₄. After filtration, the filtrate was concentrated under reduced pressure. The crude product was purified by Prep-TLC PE/EA (1:1) to afford tert-butyl N-[(3R)-1-(2-{8-cyano-2-methylimidazo[1,2-a]pyridin-6-yl}pyrido[3,2-d]pyrimidin-6-yl) pyrrolidin-3-yl]-N-methylcarbamate (80 mg, 99%) as a solid. LCMS: 485[M+H]⁺

### Synthesis of Compound 407

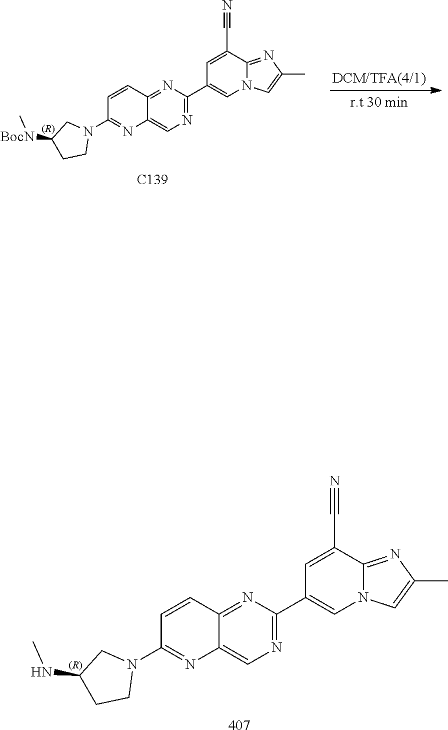

Into a 50 mL round-bottom flask were added tert-butyl N-[(3R)-1-(2-{8-cyano-2-methylimidazo[1,2-a]pyridin-6-yl}pyrido[3,2-d]pyrimidin-6-yl)pyrrolidin-3-yl]-N-methylcarbamate (50 mg, 0.10 mmol, 1 equiv). TFA (1 mL, 13.46 mmol, 130.47 equiv) and DCM (4 mL). The resulting mixture was stirred for 30 min at room temperature. The mixture was concentrated under reduced pressure. The crude product was purified by Prep-HPLC (Condition 11, Gradient 2) to afford 2-methyl-6-{6-[(3R)-3-(methylamino)pyrrolidin-1-yl]pyrido[3,2-d]pyrimidin-2-yl}imidazo[1,2-a]pyridine-8-carbonitrile (3.0 mg, 7%) as a solid. LCMS: 385[M+H]^{+ 1}H NMR (400 MHz, DMSO-d₆) δ 9.79 (d, J=1.6 Hz, 1H), 9.22 (s, 1H), 8.70 (d, J=1.6 Hz, 1H), 8.16-8.06 (m, 2H), 7.44 (d, J=9.5 Hz, 1H), 3.76 (s, 1H), 3.65 (s, 2H), 3.55 (s, 1H), 3.45 (s, 1H), 2.67(3H), 2.45-2.38 (3H), 2.19 (s, 1H), 1.98 (s, 1H).

### Example 158: Synthesis of Compound 415

### Synthesis of Intermediate C140

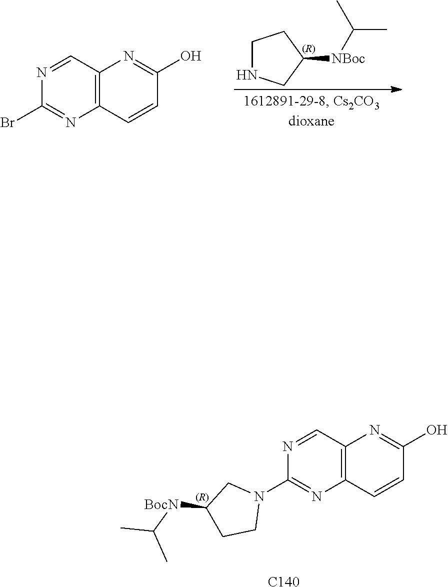

To a stirred solution of 2-bromopyrido[3,2-d]pyrimidin-6-ol (250 mg, 1.106 mmol, 1 equiv), Cs₂CO₃ (1081 mg, 3.318 mmol, 3 equiv) and tert-butyl N-isopropyl-N-[(3R)-pyrrolidin-3-yl]carbamate (303 mg, 1.327 mmol, 1.2 equiv) in dioxane (5 mL) was added Pd-PEPPSI-IPentCl 2-methylpyridine (o-picoline) (46 mg, 0.055 mmol, 0.05 equiv) at room temperature under nitrogen atmosphere. The resulting mixture was stirred for 3 h at 100° C. The resulting mixture was concentrated under reduced pressure. The residue was purified by reverse flash chromatography to afford tert-butyl N-[(3R)-1-{6-hydroxypyrido[3,2-d]pyrimidin-2-yl}pyrrolidin-3-yl]-N-isopropylcarbamate (150 mg, 36%) as a solid. LCMS (ES, m/z): 374 [M+H]⁺

### Synthesis of Intermediate C141

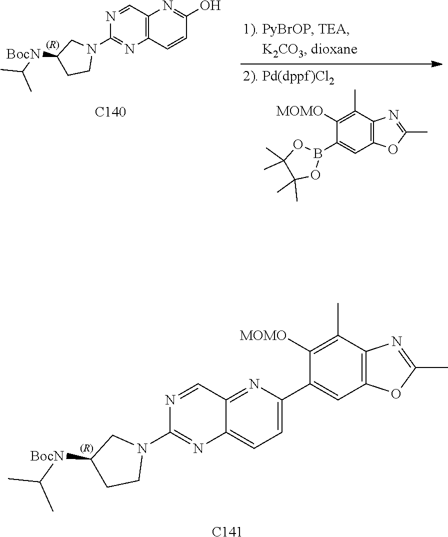

To a stirred solution of tert-butyl N-cyclopropyl-N-[(3R)-1-{6-hydroxypyrido[3,2-d]pyrimidin-2-yl}pyrrolidin-3-yl]carbamate (150 mg, 0.404 mmol, 1 equiv) and TEA (122 mg, 1.212 mmol, 3 equiv) in dioxane (10 mL) were added K₂CO₃ (167 mg, 1.212 mmol, 3 equiv) and PyBrOP (282.39 mg, 0.606 mmol, 1.5 equiv) at room temperature under nitrogen atmosphere. The resulting mixture was stirred for 3 hr at 100° C. To the above mixture were added Pd(dppf)Cl₂ (29 mg, 0.040 mmol, 0.1 equiv), 5-(methoxymethoxy)-2,4-dimethyl-6-(4,4,5,5-tetramethyl-1,3,2-dioxaborolan-2-yl)-1,3-benzoxazole (269 mg, 0.808 mmol, 2 equiv) and H₂O (0.5 mL) at room temperature. The resulting mixture was stirred for additional 2 hr at 100° C. The mixture was purified by reverse flash chromatography (Condition 10, Gradient 1) to afford tert-butyl N-isopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (300 mg, 39.82%) and tert-butyl N-cyclopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (120 mg, 53.00%)) as a solid. LCMS (ES, m/z): 561 [M+H]⁺

### Synthesis of Compound 415

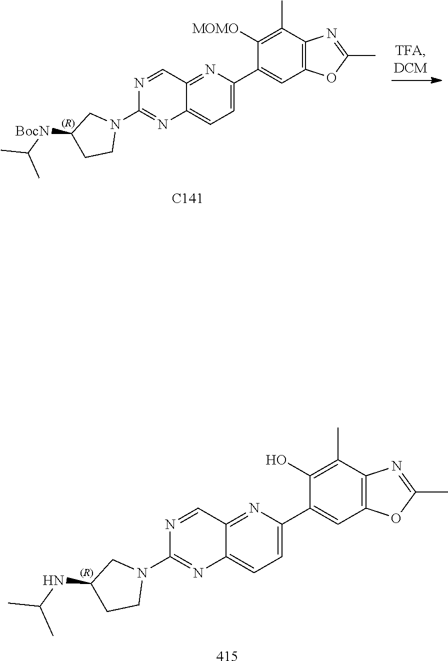

A solution of tert-butyl N-cyclopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (120 mg, 0.214 mmol, 1 equiv) and trifluoroacetic acid (2 mL) in DCM (10 mL) was stirred for 3 h at room temperature. The resulting mixture was concentrated under vacuum. The reaction was quenched by the addition of NH₃(g) in methanol (2 mL) at room temperature. The resulting mixture was concentrated under reduced pressure. The residue was purified by reverse flash chromatography (Condition 10, Gradient 1) to afford 6-{2-[(3R)-3-(cyclopropylamino)pyrrolidin-1-yl]pyrido[3,2-d]pyrimidin-6-yl}-2,4-dimethyl-1,3-benzoxazol-5-ol (15 mg, 16%) as a solid. LCMS (ES, m/z): 417 [M+H]^{+ 1}H NMR (400 MHz, DMSO-d₆) δ 14.33 (s, 1H), 9.32 (s, 1H), 8.57 (d, J=9.3 Hz, 1H), 8.25 (s, 1H), 8.06 (d, J=9.2 Hz, 1H), 3.77 (s, 2H), 3.62 (s, 2H), 3.50 (s, 2H), 2.62 (s, 3H), 2.56 (s, 3H), 2.40 (s, 2H), 1.9-1.90 (m, 1H), 0.43 (d, J=6.7 Hz, 2H), 0.27 (s, 2H).

### Example 159: Synthesis of Compound 433

### Synthesis of Intermediate C142

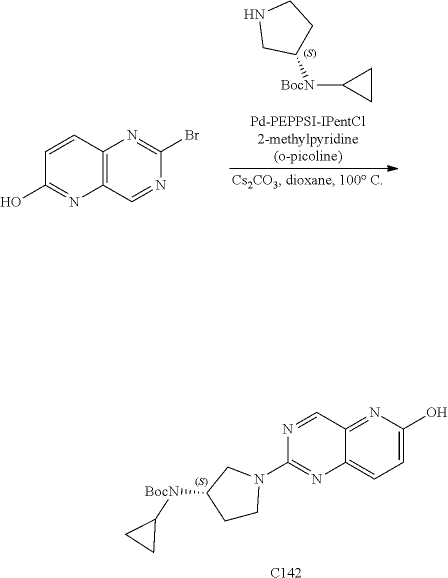

To a stirred solution of 2-bromopyrido[3,2-d]pyrimidin-6-ol (1 g, 4.424 mmol, 1 equiv) and tert-butyl N-cyclopropyl-N-[(3S)-pyrrolidin-3-yl]carbamate (1.2 g, 5.309 mmol, 1.2 equiv) in dioxane (10 mL) were added Cs₂CO₃ (2.9 g, 8.848 mmol, 2 equiv) and Pd-PEPPSI-IPentCl 2-methylpyridine (o-picoline (0.2 g, 0.221 mmol, 0.05 equiv) in portions at room temperature under nitrogen atmosphere. The resulting mixture was stirred for 1 hr at 80° C. under nitrogen atmosphere. The mixture was allowed to cool down to room temperature. The reaction was quenched by the addition of water (20 mL) at room temperature. The resulting mixture was extracted with EtOAc (3×5 mL). The combined organic layers were washed with brine (1×20 mL), dried over anhydrous Na₂SO₄. After filtration, the filtrate was concentrated under reduced pressure. The residue was purified by silica gel column chromatography, eluted with PE/EA (1:1) to afford tert-butyl N-cyclopropyl-N-[(3S)-1-{6-hydroxypyrido[3,2-d]pyrimidin-2-yl}pyrrolidin-3-yl]carbamate (500 mg, 30%) as a solid. LCMS (ES, m/z): 370 [M−H]⁻

### Synthesis of Intermediate C143

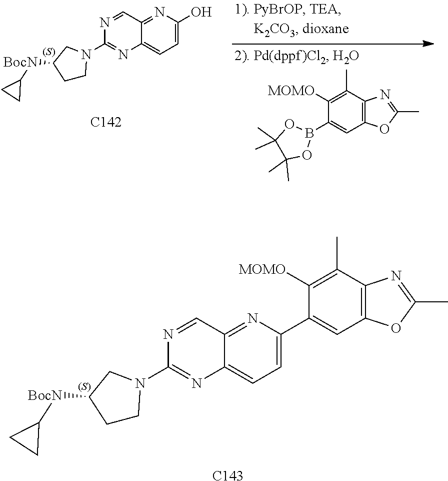

To a stirred solution of tert-butyl N-cyclopropyl-N-[(3S)-1-{6-hydroxypyrido[3,2-d]pyrimidin-2-yl}pyrrolidin-3-yl]carbamate (150 mg, 0.404 mmol, 1 equiv) and PyBrOP (315 mg, 0.606 mmol, 1.5 equiv) in dioxane (10 mL) were added K₂CO₃ (167 mg, 1.212 mmol, 3 equiv) and TEA (123 mg, 1.212 mmol, 3 equiv) in portions at room temperature under nitrogen atmosphere. The resulting mixture was stirred for 3 hr at 100° C. under nitrogen atmosphere. To the above mixture were added H₂O (0.2 mL), 5-(methoxymethoxy)-2,4-dimethyl-6-(4,4,5,5-tetramethyl-1,3,2-dioxaborolan-2-yl)-1,3-benzoxazole (270 mg, 0.808 mmol, 2 equiv) and Pd(dppf)Cl₂ (30 mg, 0.040 mmol, 0.1 equiv) in portions at room temperature. The resulting mixture was stirred for additional 4 hr at 100° C. The mixture was allowed to cool down to room temperature. The resulting mixture was concentrated under vacuum. The residue was purified by silica gel column chromatography, eluted with PE/EA (1:10) to afford tert-butyl N-cyclopropyl-N-[(3S)-1-{6-[5-(methoxymethoxy)-2,4-dimethyl-1,3-benzoxazol-6-yl]pyrido[3,2-d]pyrimidin-2-yl}pyrrolidin-3-yl]carbamate (100 mg, 44%) as a solid. LCMS (ES, m/z): 561 [M+H]⁺

### Synthesis of Compound 433

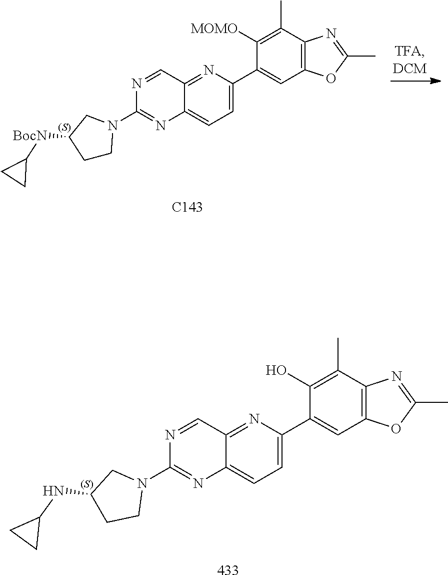

A solution of tert-butyl N-cyclopropyl-N-[(3S)-1-{6-[5-(methoxymethoxy)-2,4-dimethyl-1,3-benzoxazol-6-yl]pyrido[3,2-d]pyrimidin-2-yl}pyrrolidin-3-yl]carbamate (100 mg, 0.178 mmol, 1 equiv) in TFA (2 mL) and DCM (2 mL) was stirred for 2 hr at room temperature. The resulting mixture was concentrated under vacuum. The residue was basified to pH 8 with 7 M NH₃(g) in MeOH. The resulting mixture was concentrated under reduced pressure. The residue was purified by reverse flash chromatography (Condition 10, Gradient 2) to afford 6-{2-[(3S)-3-(cyclopropylamino)pyrrolidin-1-yl]pyrido[3,2-d]pyrimidin-6-yl}-2,4-dimethyl-1,3-benzoxazol-5-ol (35 mg, 47.12%) as a yellow solid. LCMS (ES, m/z): 417 [M+H]^{+ 1}H NMR (400 MHz, DMSO-d₆) δ 14.35 (s, 1H), 9.36 (s, 1H), 8.60 (d, J=9.3 Hz, 1H), 8.29 (s, 1H), 8.08 (d, J=9.1 Hz, 1H), 3.79 (s, 1H), 3.65 (s, 3H), 3.51 (s, 2H), 2.63 (s, 3H), 2.41 (s, 3H), 2.12 (s, 3H), 1.93 (s, 1H), 1.24 (s, 1H), 0.42 (d, J=6.6 Hz, 2H), 0.25 (s, 2H).

### Example 160: Synthesis of Compound 435

### Synthesis of Intermediate C144

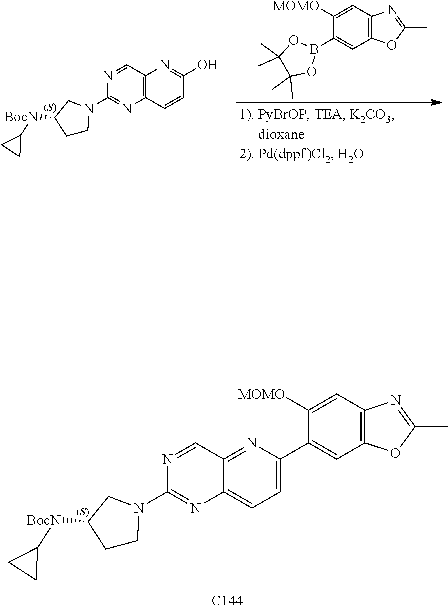

To a stirred solution of tert-butyl N-cyclopropyl-N-[(3S)-1-{6-hydroxypyrido[3,2-d]pyrimidin-2-yl}pyrrolidin-3-yl]carbamate (150 mg, 0.404 mmol, 1 equiv) and PyBrOP (283 mg, 0.606 mmol, 1.5 equiv) in dioxane (10 mL) were added K₂CO₃ (168 mg, 1.212 mmol, 3 equiv) and TEA (123 mg, 1.212 mmol, 3 equiv) in portions at room temperature under nitrogen atmosphere. The resulting mixture was stirred for 2 hr at 100° C. under nitrogen atmosphere. The mixture was allowed to cool down to room temperature. To the above mixture were added H₂O (0.2 mL), 5-(methoxymethoxy)-2-methyl-6-(4,4,5,5-tetramethyl-1,3,2-dioxaborolan-2-yl)-1,3-benzoxazole (258 mg, 0.808 mmol, 2 equiv) and Pd(dppf)Cl₂ (30 mg, 0.04 mmol, 0.1 equiv) in portions at room temperature. The resulting mixture was stirred for additional 3 hr at 100° C. The mixture was allowed to cool down to room temperature. The resulting mixture was concentrated under vacuum. The residue was purified by silica gel column chromatography, eluted with PE/EA (1:10) to afford tert-butyl N-cyclopropyl-N-[(3S)-1-{6-[5-(methoxymethoxy)-2-methyl-1,3-benzoxazol-6-yl]pyrido[3,2-d]pyrimidin-2-yl}pyrrolidin-3-yl]carbamate (100 mg, 45%) as a solid. LCMS (ES, m/z): 547 [M+H]⁺

### Synthesis of Compound 435

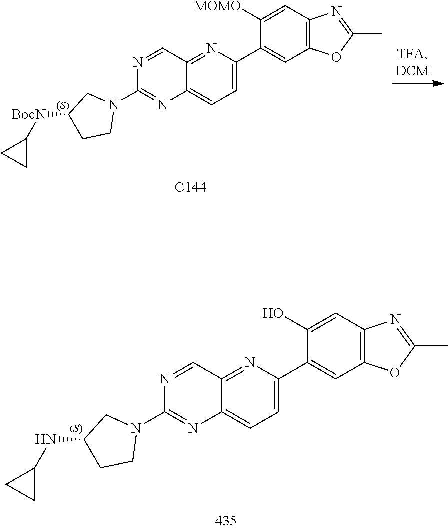

A solution of tert-butyl N-cyclopropyl-N-[(3S)-1-{6-[5-(methoxymethoxy)-2-methyl-1,3-benzoxazol-6-yl]pyrido[3,2-d]pyrimidin-2-yl}pyrrolidin-3-yl]carbamate (100 mg, 0.183 mmol, 1 equiv) in TFA (2 mL) and DCM (2 mL) was stirred for 2 hr at room temperature. The resulting mixture was concentrated under vacuum. The residue was basified to pH 8 with 7 M NH₃(g) in MeOH. The resulting mixture was concentrated under reduced pressure. The residue was purified by reverse flash chromatography (Condition 10, Gradient 4) to afford 6-{2-[(3S)-3-(cyclopropylamino)pyrrolidin-1-yl]pyrido[3,2-d]pyrimidin-6-yl}-2-methyl-1,3-benzoxazol-5-ol (20 mg, 27%) as a solid. LCMS (ES, m/z): 403 [M+H]^{+ 1}H NMR (400 MHz, DMSO-d₆) δ 13.58 (s, 1H), 9.32 (s, 1H), 8.57 (d, J=9.3 Hz, 1H), 8.40 (s, 1H), 8.07 (d, J=9.2 Hz, 1H), 7.15 (s, 1H), 3.65 (d, J=101.0 Hz, 5H), 2.62 (s, 3H), 2.14 (s, 2H), 1.98-1.90 (m, 1H), 0.43 (d, J=6.8 Hz, 2H), 0.28 (s, 2H).

### Example 161: Synthesis of Compound 438

### Synthesis of Intermediate C145

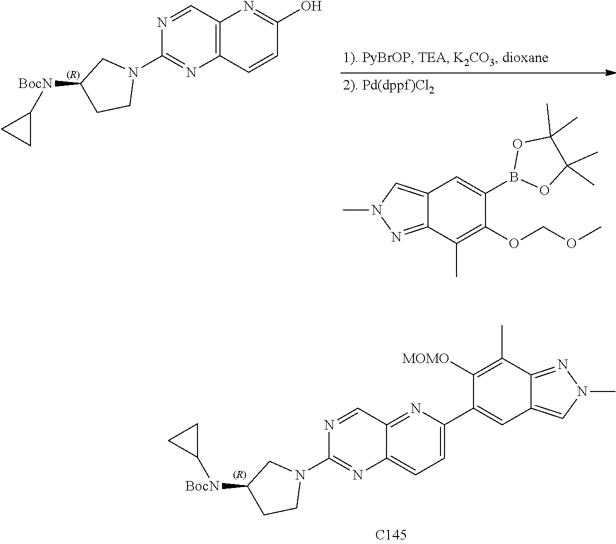

To a stirred solution of tert-butyl N-cyclopropyl-N-[(3R)-1-{6-hydroxypyrido[3,2-d]pyrimidin-2-yl}pyrrolidin-3-yl]carbamate (150 mg, 0.404 mmol, 1 equiv) and TEA (122 mg, 1.212 mmol, 3 equiv) in dioxane (10 mL) were added K₂CO₃ (167 mg, 1.212 mmol, 3 equiv) and PyBrOP (282.39 mg, 0.606 mmol, 1.5 equiv) at room temperature under nitrogen atmosphere. The resulting mixture was stirred for 3 hr at 100° C. To the above mixture were added Pd(dppf)Cl₂ (29 mg, 0.040 mmol, 0.1 equiv), 6-(methoxymethoxy)-2,7-dimethyl-5-(4,4,5,5-tetramethyl-1,3,2-dioxaborolan-2-yl) indazole (268.31 mg, 0.808 mmol, 2 equiv) and H₂O (0.5 mL) at room temperature. The resulting mixture was stirred for additional 2 hr at 100° C. The resulting mixture was purified by reverse flash chromatography (Condition 10, Gradient 1) to afford tert-butyl N-cyclopropyl-N-[(3R)-1-{6-[6-(methoxymethoxy)-2,7-dimethylindazol-5-yl]pyrido[3,2-d]pyrimidin-2-yl}pyrrolidin-3-yl]carbamate (120 mg, 53%) as a solid. LCMS (ES, m/z): 560 [M+H]⁺

### Synthesis of Compound 438

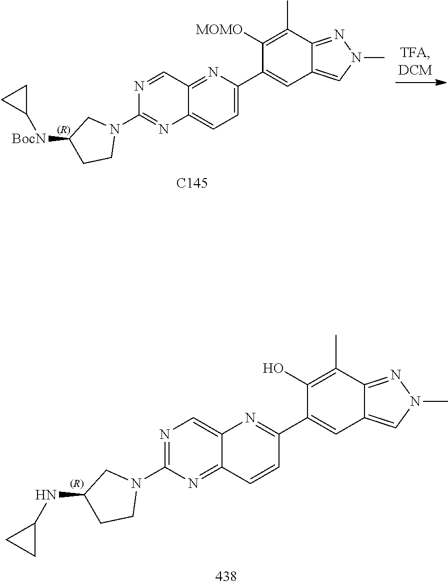

A solution of tert-butyl N-cyclopropyl-N-[(3R)-1-{6-[6-(methoxymethoxy)-2,7-dimethylindazol-5-yl]pyrido[3,2-d]pyrimidin-2-yl}pyrrolidin-3-yl]carbamate (120 mg, 0.214 mmol, 1 equiv) and trifluoroacetic acid (2 mL) in DCM (10 mL) was stirred for 3 h at room temperature. The resulting mixture was concentrated under vacuum. The residue was purified by reverse flash chromatography (Condition 10, Gradient 5) to afford 5-{2-[(3R)-3-(cyclopropylamino)pyrrolidin-1-yl]pyrido[3,2-d]pyrimidin-6-yl}-2,7-dimethylindazol-6-ol (35 mg, 39%) as a solid. LCMS (ES, m/z): 416 [M+H]¹H NMR (400 MHz, DMSO-d₆) δ 13.65 (s, 1H), 9.30 (s, 1H), 8.56 (d, J=9.4 Hz, 1H), 8.42 (s, 1H), 8.35 (s, 1H), 8.04 (d, J=9.2 Hz, 1H), 4.14 (s, 3H), 3.77 (s, 2H), 3.62 (s, 1H), 3.50 (s, 3H), 2.39 (s, 3H), 2.12 (s, 2H), 1.96-1.88 (m, 1H), 0.42 (d, J=6.8 Hz, 2H), 0.29-0.22 (m, 2H).

### Example 162: Synthesis of Compound 439

### Synthesis of Intermediate C146

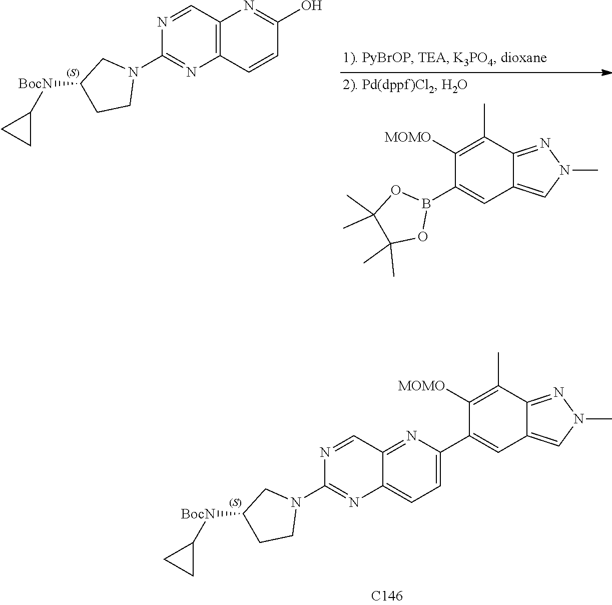

To a stirred solution of tert-butyl N-cyclopropyl-N-[(3S)-1-{6-hydroxypyrido[3,2-d]pyrimidin-2-yl}pyrrolidin-3-yl]carbamate (150 mg, 0.404 mmol, 1 equiv) and PyBrOP (315 mg, 0.606 mmol, 1.5 equiv) in dioxane (10 mL) were added K₂CO₃ (168 mg, 1.212 mmol, 3 equiv) and TEA (123 mg, 1.212 mmol, 3 equiv) in portions at room temperature under nitrogen atmosphere. The resulting mixture was stirred for 2 hr at 100° C. under nitrogen atmosphere. The mixture was allowed to cool down to room temperature. To the above mixture were added 6-(methoxymethoxy)-2,7-dimethyl-5-(4,4,5,5-tetramethyl-1,3,2-dioxaborolan-2-yl) indazole (269 mg, 0.808 mmol, 2 equiv), Pd(dppf)Cl₂ (30 mg, 0.040 mmol, 0.1 equiv) and water (0.2 mL) in portions at room temperature. The resulting mixture was stirred for additional 3 h at 100° C. The mixture was allowed to cool down to room temperature. The resulting mixture was concentrated under vacuum. The residue was purified by silica gel column chromatography, eluted with PE/EA (1:10) to afford tert-butyl N-cyclopropyl-N-[(3S)-1-{6-[6-(methoxymethoxy)-2,7-dimethylindazol-5-yl]pyrido[3,2-d]pyrimidin-2-yl}pyrrolidin-3-yl]carbamate (100 mg, 44%) as a solid. LCMS (ES, m/z): 560 [M+H]⁺

### Synthesis of Compound 439

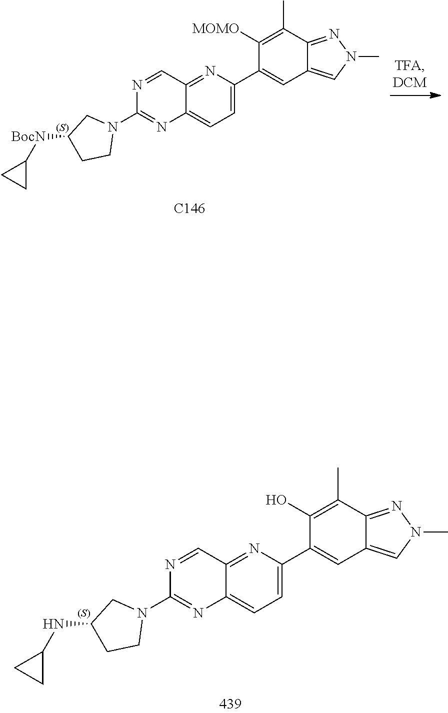

A solution of tert-butyl N-cyclopropyl-N-[(3S)-1-{6-[6-(methoxymethoxy)-2,7-dimethylindazol-5-yl]pyrido[3,2-d]pyrimidin-2-yl}pyrrolidin-3-yl]carbamate (80 mg, 0.143 mmol, 1 equiv) in TFA (2 mL) and DCM (2 mL) was stirred for 2 hr at room temperature. The resulting mixture was concentrated under vacuum. The residue was basified to pH 8 with 7 M NH₃(g) in MeOH. The resulting mixture was concentrated under reduced pressure. The residue was purified by reverse flash chromatography (Condition 10, Gradient 3) to afford 5-{2-[(3S)-3-(cyclopropylamino)pyrrolidin-1-yl]pyrido[3,2-d]pyrimidin-6-yl}-2,7-dimethylindazol-6-ol (25 mg, 42%) as a solid. LCMS (ES, m/z): 416 [M+H]^{+ 1}H NMR (300 MHz, DMSO-d₆) δ 13.66 (s, 1H), 9.32 (s, 1H), 8.58 (s, 1H), 8.44 (s, 1H), 8.36 (s, 1H), 8.06 (d, J=8.8 Hz, 1H), 4.14 (s, 3H), 3.75 (s, 1H), 3.68 (s, 2H), 3.50 (s, 2H), 2.39 (s, 3H), 2.12 (s, 2H), 1.93 (s, 1H), 1.74 (s, 1H), 0.46-0.38 (m, 2H), 0.25 (s, 2H).

### Example 163: Synthesis of Compound 440

### Synthesis of Intermediate C147

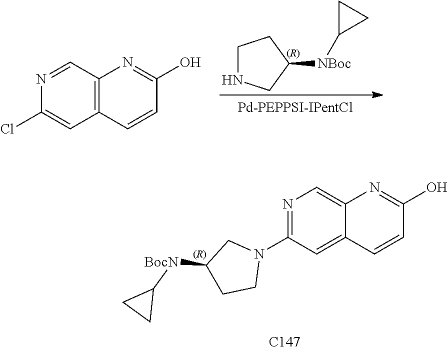

To a stirred solution of 6-chloro-1,7-naphthyridin-2-ol (150 mg, 0.831 mmol, 1 equiv) and tert-butyl N-cyclopropyl-N-[(3R)-pyrrolidin-3-yl]carbamate (281 mg, 1.246 mmol, 1.5 equiv) in dioxane (5 mL) were added Pd-PEPPSI-IPentCl 2-methylpyridine (o-picoline) (34 mg, 0.042 mmol, 0.05 equiv) and Cs₂CO₃ (811 mg, 2.493 mmol, 3 equiv) at room temperature under nitrogen atmosphere. The resulting mixture was stirred for 3 h at 100° C. The resulting mixture was concentrated under reduced pressure. The residue was purified by reverse flash chromatography with the following conditions: column, C18 silica gel; mobile phase, MeCN in Water (0.1% NH₃·H₂O+10 mmol/L NH₄HCO₃), 10% to 50% gradient in 12 min; detector, UV 254 nm to afford tert-butyl N-cyclopropyl-N-[(3R)-1-{6-hydroxypyrido[3,2-d]pyrimidin-2-yl}pyrrolidin-3-yl]carbamate (150 mg, 48%) as a solid. LCMS (ES, m/z): 371 [M+H]⁺

### Synthesis of Intermediate C148

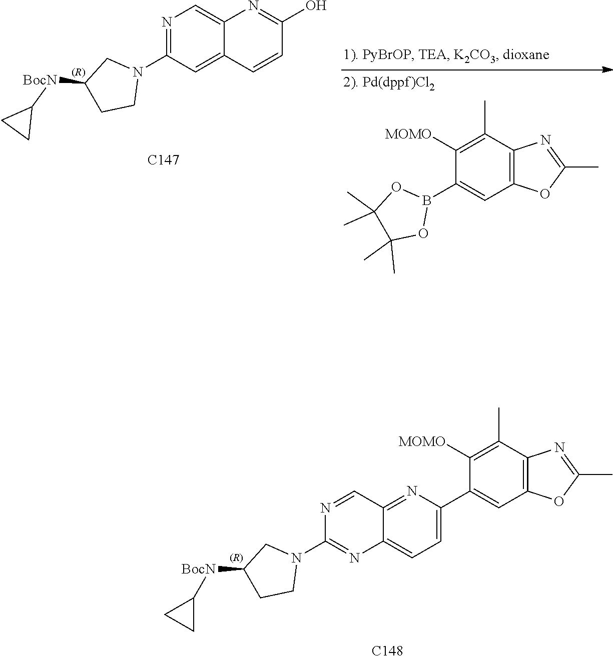

To a stirred solution of tert-butyl N-cyclopropyl-N-[(3R)-1-(2-hydroxy-1,7-naphthyridin-6-yl)pyrrolidin-3-yl]carbamate (150 mg, 0.405 mmol, 1 equiv) and TEA (122 mg, 1.215 mmol, 3 equiv) in dioxane (10 mL) were added K₂CO₃ (167 mg, 1.215 mmol, 3 equiv) and PyBrOP (377 mg, 0.810 mmol, 2 equiv) at room temperature under nitrogen atmosphere. The resulting mixture was stirred for 3 hr at 100° C. To the above mixture were added Pd(dppf)Cl₂ (29 mg, 0.041 mmol, 0.1 equiv), 5-(methoxymethoxy)-2,4-dimethyl-6-(4,4,5,5-tetramethyl-1,3,2-dioxaborolan-2-yl)-1,3-benzoxazole (269 mg, 0.810 mmol, 2 equiv) and H₂O (0.5 mL) at room temperature. The resulting mixture was stirred for additional 2 hr at 100° C. The resulting mixture was concentrated under reduced pressure. The residue was purified by reverse flash chromatography (Condition 10, Gradient 1) to afford tert-butyl N-cyclopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (120 mg, 52%) as a solid. LCMS (ES, m/z): 560 [M+H]⁺

### Synthesis of Compound 440

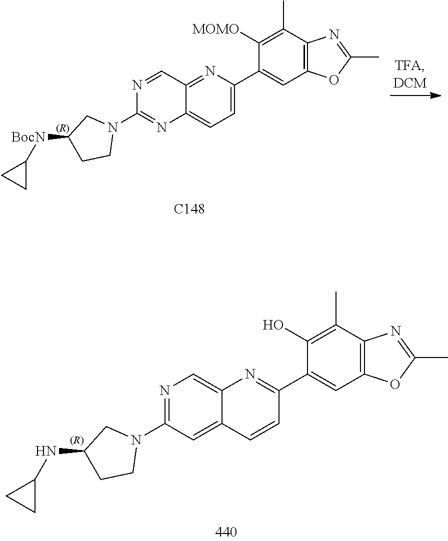

A solution of tert-butyl N-cyclopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (120 mg, 0.214 mmol, 1 equiv) and trifluoroacetic acid (1 mL) in DCM (5 mL) was stirred for 2 h at room temperature. The resulting mixture was concentrated under vacuum. The residue was purified by reverse flash chromatography (Condition 10, Gradient 4) to afford 6-{6-[(3R)-3-(cyclopropylamino)pyrrolidin-1-yl]-1,7-naphthyridin-2-yl}-2,4-dimethyl-1,3-benzoxazol-5-ol (32 mg, 36%) as a solid. LCMS (ES, m/z): 416 [M+H]^{+ 1}H NMR (400 MHz, DMSO-d6) δ 14.89 (s, 1H), 9.07 (s, 1H), 8.37 (d, J=9.3 Hz, 1H), 8.29-8.22 (m, 2H), 6.64 (s, 1H), 3.69 (dd, J=10.4, 6.0 Hz, 1H), 3.52 (ddq, J=25.3, 17.5, 9.0, 8.1 Hz, 3H), 3.35 (d, J=5.0 Hz, 1H), 2.62 (s, 3H), 2.41 (s, 3H), 2.23-2.06 (m, 2H), 1.93 (dq, J=13.0, 6.7 Hz, 1H), 0.42 (dd, J=6.5, 1.7 Hz, 2H), 0.25 (dt, J=10.2, 6.0 Hz, 2H).

### Example 164: Synthesis of Compound 441

### Synthesis of Intermediate C149

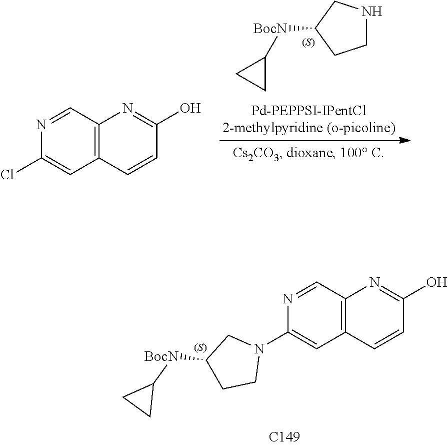

To a stirred solution of 6-chloro-1,7-naphthyridin-2-ol (130 mg, 0.72 mmol, 1 equiv) and tert-butyl N-cyclopropyl-N-[(3S)-pyrrolidin-3-yl]carbamate (245 mg, 1.08 mmol, 1.5 equiv) in dioxane (10 mL) were added Cs₂CO₃ (469 mg, 1.44 mmol, 2 equiv) and Pd-PEPPSI-IPentCl 2-methylpyridine (o-picoline) (5 mg, 0.006 mmol, 0.05 equiv) in portions at room temperature under nitrogen atmosphere. The resulting mixture was stirred for 3 hr at 100° C. under nitrogen atmosphere. The mixture was allowed to cool down to room temperature. The resulting mixture was concentrated under vacuum. The residue was purified by silica gel column chromatography, eluted with PE/EA (1:10) to afford tert-butyl N-cyclopropyl-N-[(3S)-1-(2-hydroxy-1,7-naphthyridin-6-yl)pyrrolidin-3-yl]carbamate (120 mg, 45%) as a solid. LCMS (ES, m/z): 371 [M+H]⁺

### Synthesis of Intermediate C50

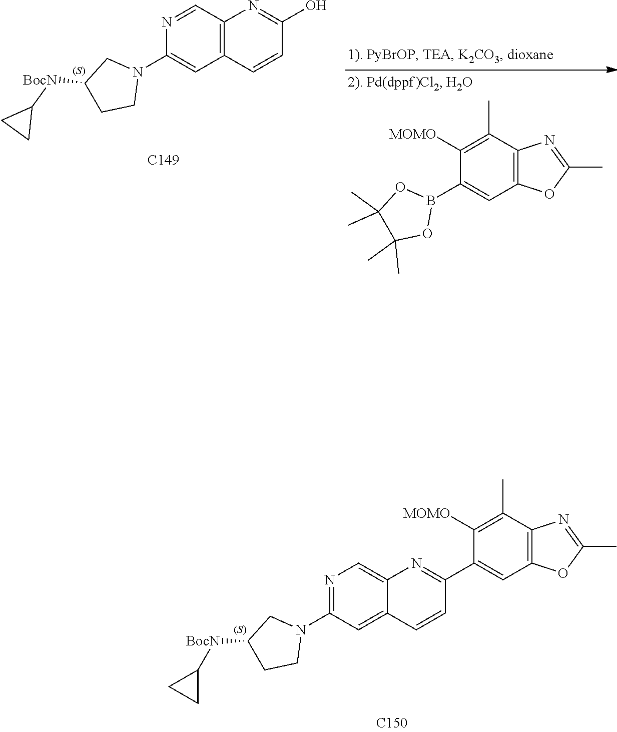

To a stirred solution of tert-butyl N-cyclopropyl-N-[(3S)-1-(2-hydroxy-1,7-naphthyridin-6-yl)pyrrolidin-3-yl]carbamate (120 mg, 0.324 mmol, 1 equiv) and PyBrOP (227 mg, 0.486 mmol, 1.5 equiv) in dioxane (10 mL) were added K₂CO₃ (135 mg, 0.972 mmol, 3 equiv) and TEA (99 mg, 0.972 mmol, 3 equiv) in portions at room temperature under nitrogen atmosphere. The resulting mixture was stirred for 2 hr at 100° C. under nitrogen atmosphere. The mixture was allowed to cool down to room temperature. To the above mixture was added H₂O (0.2 mL), 5-(methoxymethoxy)-2,4-dimethyl-6-(4,4,5,5-tetramethyl-1,3,2-dioxaborolan-2-yl)-1,3-benzoxazole (216 mg, 0.648 mmol, 2 equiv) and Pd(dppf)Cl₂ (24 mg, 0.032 mmol, 0.1 equiv) in portions at room temperature. The resulting mixture was stirred for additional 3 h at 100° C. The mixture was allowed to cool down to room temperature. The resulting mixture was concentrated under vacuum. The residue was purified by silica gel column chromatography, eluted with PE/EA (1:10) to afford tert-butyl N-cyclopropyl-N-[(3S)-1-{2-[5-(methoxymethoxy)-2,4-dimethyl-1,3-benzoxazol-6-yl]-1,7-naphthyridin-6-yl}pyrrolidin-3-yl]carbamate (90 mg, 50%) as a solid. LCMS (ES, m/z): 560 [M+H]⁺

### Synthesis of Compound 441

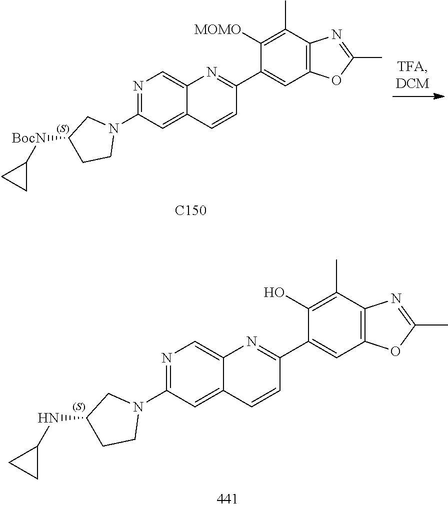

A mixture of tert-butyl N-cyclopropyl-N-[(3S)-1-{2-[5-(methoxymethoxy)-2,4-dimethyl-1,3-benzoxazol-6-yl]-1,7-naphthyridin-6-yl}pyrrolidin-3-yl]carbamate (90 mg, 0.161 mmol, 1 equiv) in DCM (3 mL) and TFA (1 mL) was stirred for 3 hr at room temperature. The resulting mixture was concentrated under reduced pressure. The residue was basified to pH 8 with 7 M NH₃(g) in MeOH. The resulting mixture was concentrated under vacuum. The residue was purified by reverse flash chromatography (Condition 10, Gradient 6) to afford 6-{6-[(3S)-3-(cyclopropylamino)pyrrolidin-1-yl]-1,7-naphthyridin-2-yl}-2,4-dimethyl-1,3-benzoxazol-5-ol (57 mg, 85%) as a solid. LCMS (ES, m/z): 416 [M+H]^{+ 1}H NMR (300 MHz, DMSO-d₆) δ 14.88 (s, 1H), 9.07 (s, 1H), 8.36 (d, J=8.8 Hz, 1H), 8.24 (d, J=10.5 Hz, 2H), 6.64 (s, 1H), 3.69 (s, 1H), 3.50 (d, J=6.7 Hz, 3H), 3.31 (s, 1H), 2.62 (s, 3H), 2.41 (s, 3H), 2.14 (s, 2H), 1.93 (s, 1H), 0.41 (d, J=6.7 Hz, 2H), 0.25 (s, 2H).

### Example 165: Synthesis of Compounds 413 and 414

### Synthesis of Intermediate C151

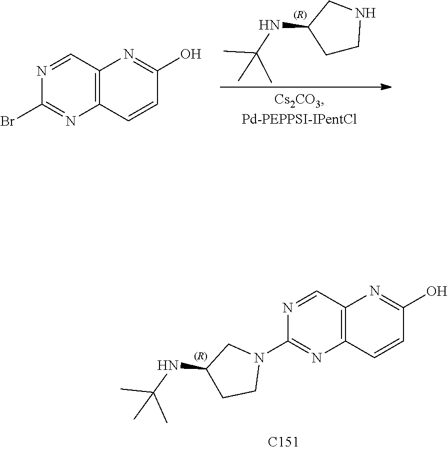

To a stirred solution of 2-bromopyrido[3,2-d]pyrimidin-6-ol (250 mg, 1.106 mmol, 1 equiv), Cs₂CO₃ (1081.10 mg, 3.318 mmol, 3 equiv) and (3R)—N-tert-butylpyrrolidin-3-amine (235 mg, 1.659 mmol, 1.5 equiv) in dioxane (5 mL) was added Pd-PEPPSI-IPentCl 2-methylpyridine (o-picoline) (46 mg, 0.055 mmol, 0.05 equiv) at room temperature under nitrogen atmosphere. The resulting mixture was stirred for 3 h at 100° C. The resulting mixture was concentrated under reduced pressure. The residue was purified by reverse flash chromatography (Condition 10, Gradient 2) to afford 2-[(3R)-3-(tert-butylamino)pyrrolidin-1-yl]pyrido[3,2-d]pyrimidin-6-ol) (150 mg, 48%) as a solid. LCMS (ES, m/z): 288 [M+H]⁺

### Synthesis of Intermediate C152

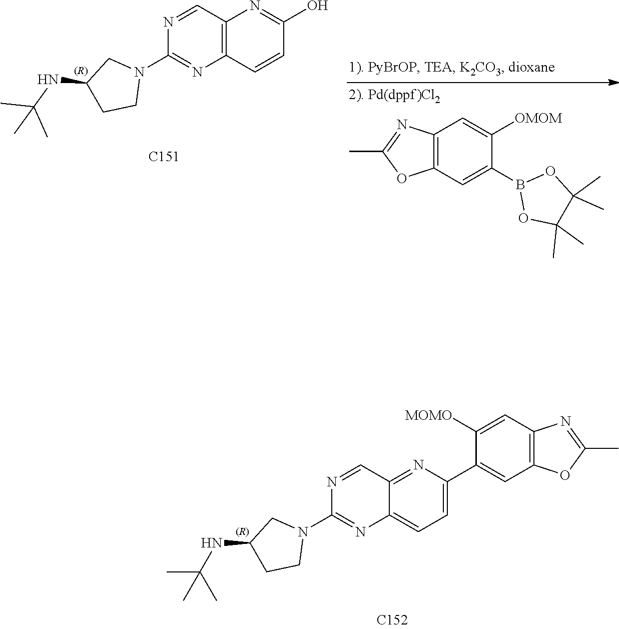

To a stirred solution of 2-[(3R)-3-(tert-butylamino)pyrrolidin-1-yl]pyrido[3,2-d]pyrimidin-6-ol (150 mg, 0.522 mmol, 1 equiv) and PyBrOP (365 mg, 0.783 mmol, 1.5 equiv) in dioxane (5 mL) were added TEA (158 mg, 1.566 mmol, 3 equiv) and K₂CO₃ (216 mg, 1.566 mmol, 3 equiv) at room temperature under nitrogen atmosphere. The resulting mixture was stirred for 3 h at 100° C. The mixture was allowed to cool down to room temperature. To the above mixture was added Pd(dppf)Cl₂ (38 mg, 0.052 mmol, 0.1 equiv), 5-(methoxymethoxy)-2-methyl-6-(4,4,5,5-tetramethyl-1,3,2-dioxaborolan-2-yl)-1,3-benzoxazole (199 mg, 0.626 mmol, 1.2 equiv) and H₂O (0.5 mL) at room temperature. The resulting mixture was stirred for additional 2 hr at 100° C. The mixture was allowed to cool down to room temperature. The resulting mixture was concentrated under reduced pressure. The residue was purified by reverse flash chromatography (Condition 9, Gradient 2) to afford (3R)-N-tert-butyl-1-{6-[5-(methoxymethoxy)-2-methyl-1,3-benzoxazol-6-yl]pyrido[3,2-d]pyrimidin-2-yl}pyrrolidin-3-amine (120 mg, 49%) as a solid. LCMS (ES, m/z): 463 [M+H]⁺

### Synthesis of Compound 413

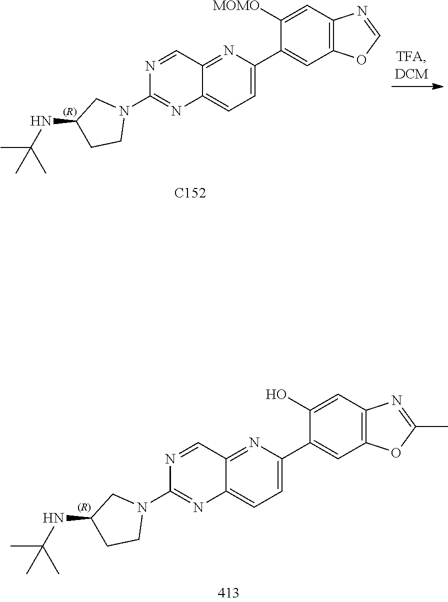

A solution of (3R)-N-tert-butyl-1-{6-[5-(methoxymethoxy)-2-methyl-1,3-benzoxazol-6-yl]pyrido[3,2-d]pyrimidin-2-yl}pyrrolidin-3-amine (120 mg, 0.267 mmol, 1 equiv) and TFA (0.80 mL) in DCM (3 mL) was stirred for 2 hr at room temperature. The resulting mixture was concentrated under reduced pressure. The residue was purified by reverse flash chromatography (Condition 10, Gradient 4) to afford 6-{2-[(3R)-3-(tert-butylamino)pyrrolidin-1-yl]pyrido[3,2-d]pyrimidin-6-yl}-2-methyl-1,3-benzoxazol-5-ol (15 mg, 13%) as a solid. LCMS (ES, m/z): 419 [M+H]^{+ 1}H NMR (400 MHz, DMSO-d₆) δ 13.54 (s, 1H), 9.36 (s, 1H), 8.59 (d, J=9.3 Hz, 1H), 8.42 (s, 1H), 8.10 (d, J=9.3 Hz, 1H), 7.16 (s, 1H), 4.00 (d, J=20.9 Hz, 1H), 3.80 (s, 2H), 3.58 (s, 2H), 2.63 (s, 3H), 2.27 (s, 1H), 1.90 (s, 1H), 1.18 (s, 9H).

### Synthesis of Intermediate C153

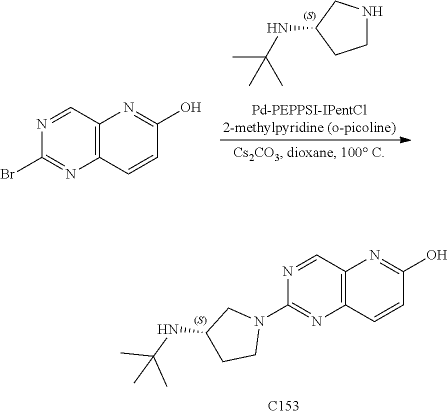

To a stirred solution of 2-bromopyrido[3,2-d]pyrimidin-6-ol (200 mg, 0.885 mmol, 1 equiv) and (3S)—N-tert-butylpyrrolidin-3-amine (151 mg, 1.062 mmol, 1.2 equiv), Cs₂CO₃ (865 mg, 2.655 mmol, 3 equiv) in dioxane (5 mL) was added Pd-PEPPSI-IPentCl 2-methylpyridine (o-picoline) (38 mg, 0.044 mmol, 0.05 equiv) at room temperature under nitrogen atmosphere. The resulting mixture was stirred for 3 h at 100° C. The mixture was allowed to cool down to room temperature. The reaction was quenched by the addition of water (20 mL) at room temperature. The resulting mixture was extracted with ethyl acetate (3×5 mL). The combined organic layers were washed with brine (1×20 mL), dried over anhydrous Na₂SO₄. After filtration, the filtrate was concentrated under reduced pressure. The residue was purified by reverse flash chromatography (Condition 9, Gradient 3) to afford 2-[(3S)-3-(tert-butylamino)pyrrolidin-1-yl]pyrido[3,2-d]pyrimidin-6-ol (100 mg, 39%) as a solid. LCMS (ES, m/z): 288 [M+H]⁺

### Synthesis of Intermediate C154

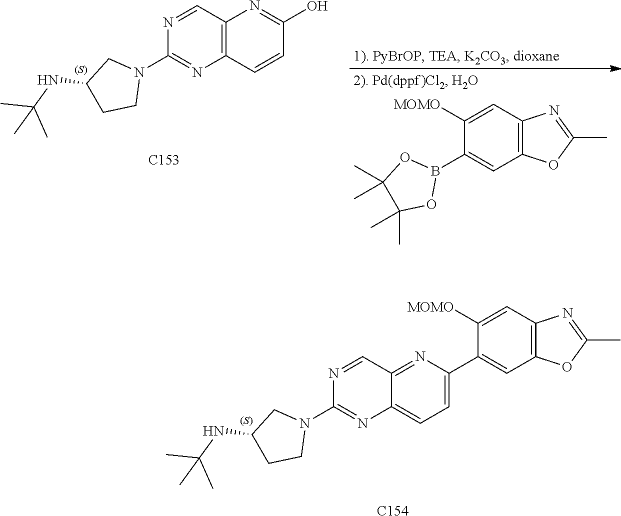

To a stirred solution of 2-[(3S)-3-(tert-butylamino)pyrrolidin-1-yl]pyrido[3,2-d]pyrimidin-6-ol (150 mg, 0.522 mmol, 1 equiv) and TEA (159 mg, 1.566 mmol, 3 equiv) in dioxane (8 mL) were added K₂CO₃ (217 mg, 1.566 mmol, 3 equiv) and PyBrOP (365 mg, 0.783 mmol, 1.5 equiv) in portions at room temperature under nitrogen atmosphere. The resulting mixture was stirred for additional 3 h at 100° C. To the above mixture were added 5-(methoxymethoxy)-2-methyl-6-(4,4,5,5-tetramethyl-1,3,2-dioxaborolan-2-yl)-1,3-benzoxazole (333 mg, 1.045 mmol, 2 equiv), Pd(dppf)Cl₂ (39 mg, 0.052 mmol, 0.1 equiv) and H₂O (0.4 mL) in portions at room temperature. The resulting mixture was stirred for additional 2 hr at 100° C. The mixture was allowed to cool down to room temperature. The reaction was quenched by the addition of water (20 mL) at room temperature. The resulting mixture was extracted with ethyl acetate (3×5 mL). The combined organic layers were washed with brine (1×20 mL), dried over anhydrous Na₂SO₄. After filtration, the filtrate was concentrated under reduced pressure. The residue was purified by reverse flash chromatography (Condition 10, Gradient 7) to afford (3S)—N-tert-butyl-1-{6-[5-(methoxymethoxy)-2-methyl-1,3-benzoxazol-6-yl]pyrido[3,2-d]pyrimidin-2-yl}pyrrolidin-3-amine (100 mg, 41%) as a solid. LCMS (ES, m/z): 463 [M+H]⁺

### Synthesis of Compound 414

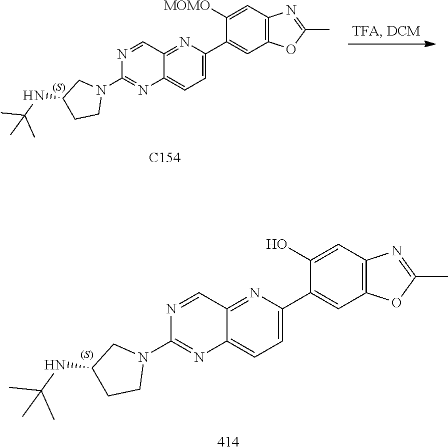

A solution of (3S)—N-tert-butyl-1-{6-[5-(methoxymethoxy)-2-methyl-1,3-benzoxazol-6-yl]pyrido[3,2-d]pyrimidin-2-yl}pyrrolidin-3-amine (100 mg, 0.216 mmol, 1 equiv) in TFA (2 mL) and DCM (2 mL) was stirred for 2 h at room temperature. The resulting mixture was concentrated under vacuum. The residue was basified to pH 8 with 7 M NH₃(g) in MeOH. The resulting mixture was concentrated under reduced pressure. The residue was purified by reverse flash chromatography (Condition 10, Gradient 2) to afford 6-{2-[(3S)-3-(tert-butylamino)pyrrolidin-1-yl]pyrido[3,2-d]pyrimidin-6-yl}-2-methyl-1,3-benzoxazol-5-ol (12 mg, 13%) as a solid. LCMS (ES, m/z): 419 [M+H]^{+ 1}H NMR (400 MHz, DMSO-d₆) δ 13.59 (s, 1H), 9.31 (s, 1H), 8.56 (d, J=9.4 Hz, 1H), 8.39 (s, 1H), 8.06 (d, J=9.2 Hz, 1H), 7.15 (s, 1H), 3.92 (s, 1H), 3.76 (s, 1H), 3.53 (s, 2H), 3.18 (s, 1H), 2.62 (s, 3H), 2.17 (s, 1H), 2.08 (s, 1H), 1.75 (d, J=11.0 Hz, 1H), 1.10 (s, 9H).

### Example 166: Synthesis of Compound 415

### Synthesis of Intermediate C155

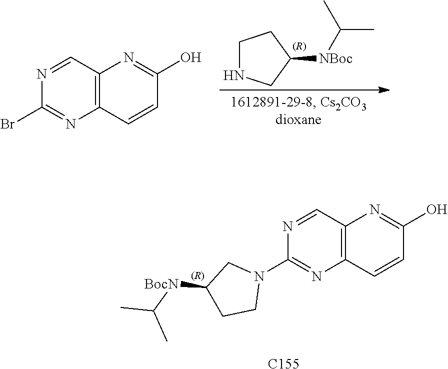

To a stirred solution of 2-bromopyrido[3,2-d]pyrimidin-6-ol (250 mg, 1.106 mmol, 1 equiv), Cs₂CO₃ (1081 mg, 3.318 mmol, 3 equiv) and tert-butyl N-isopropyl-N-[(3R)-pyrrolidin-3-yl]carbamate (303 mg, 1.327 mmol, 1.2 equiv) in dioxane (5 mL) was added Pd-PEPPSI-IPentCl 2-methylpyridine (o-picoline) (46 mg, 0.055 mmol, 0.05 equiv) at room temperature under nitrogen atmosphere. The resulting mixture was stirred for 3 hr at 100° C. The resulting mixture was concentrated under reduced pressure. The residue was purified by reverse flash chromatography (Condition 9, Gradient 1) to afford tert-butyl N-[(3R)-1-{6-hydroxypyrido[3,2-d]pyrimidin-2-yl}pyrrolidin-3-yl]-N-isopropylcarbamate (150 mg, 36%) as a solid. LCMS (ES, m/z): 374 [M+H]⁺

### Synthesis of Intermediate C156

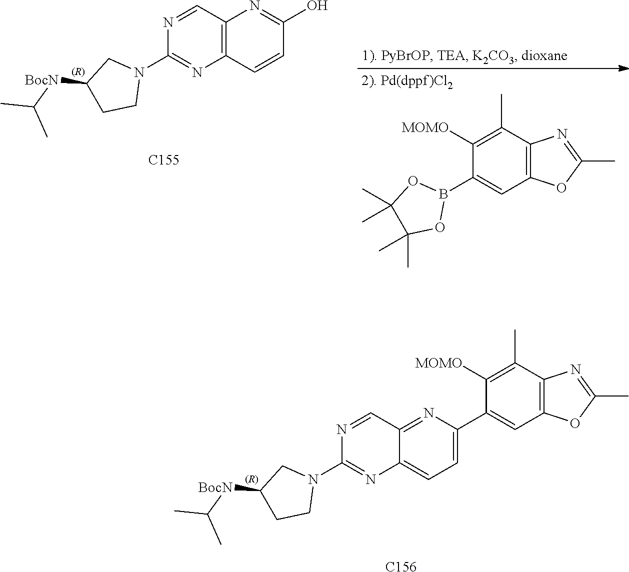

To a stirred solution of tert-butyl N-[(3R)-1-{6-hydroxypyrido[3,2-d]pyrimidin-2-yl}pyrrolidin-3-yl]-N-isopropylcarbamate (500 mg, 1.339 mmol, 1 equiv) and TEA (406 mg, 4.017 mmol, 3 equiv) in dioxane (20 mL) were added K₂CO₃ (555 mg, 4.017 mmol, 3 equiv) and PyBrOP (936 mg, 2.008 mmol, 1.5 equiv) at room temperature under nitrogen atmosphere. The resulting mixture was stirred for 3 hr at 100° C. To the above mixture were added Pd(dppf)Cl₂ (97 mg, 0.134 mmol, 0.1 equiv), 5-(methoxymethoxy)-2,4-dimethyl-6-(4,4,5,5-tetramethyl-1,3,2-dioxaborolan-2-yl)-1,3-benzoxazole (892 mg, 2.678 mmol, 2 equiv) and H₂O (1 mL) room temperature. The resulting mixture was stirred for 2 hr at 100° C. The residue was purified by reverse flash chromatography (Condition 10, Gradient 5) to afford tert-butyl N-isopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (300 mg, 39%) as a solid. LCMS (ES, m/z): 563 [M+H]⁺

### Synthesis of Compound 415

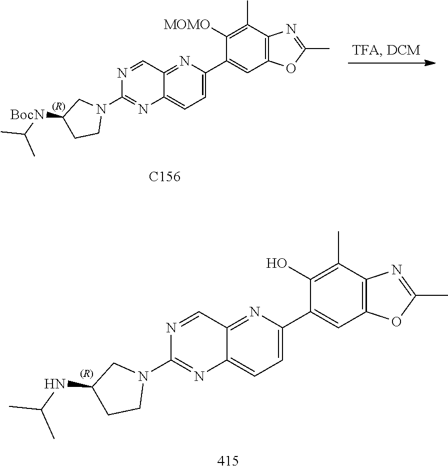

A solution of tert-butyl N-isopropyl-N-[(3R)-1-{6-[5-(methoxymethoxy)-2,4-dimethyl-1,3-benzoxazol-6-yl]pyrido 3,2-d]pyrimidin-2-yl}pyrrolidin-3-yl]carbamate (120 mg, 0.213 mmol, 1 equiv) and trifluoroacetic acid (2 mL) in DCM (10 mL) was stirred for 3 h at room temperature. The resulting mixture was concentrated under vacuum. The residue was purified by reverse flash chromatography (Condition 10, Gradient 3) to afford 6-{2-[(3R)-3-(isopropylamino)pyrrolidin-1-yl]pyrido 3,2-d]pyrimidin-6-yl}-2,4-dimethyl-1,3-benzoxazol-5-ol (15 mg, 16%) as a solid. LCMS (ES, m/z): 419 [M+H]^{+ 1}H NMR (400 MHz, DMSO-d₆) δ 14.33 (s, 1H), 9.36 (s, 1H), 8.59 (d, J=9.4 Hz, 1H), 8.28 (s, 1H), 8.08 (d, J=9.2 Hz, 1H), 3.86 (s, 1H), 3.77 (s, 1H), 3.60 (s, 2H), 2.94 (s, 1H), 2.62 (s, 3H), 2.41 (s, 3H), 2.19 (s, 2H), 1.86 (s, 1H), 1.06 (d, J=5.7 Hz, 6H)

### Example 167: Synthesis of Compound 416

### Synthesis of Intermediate C157

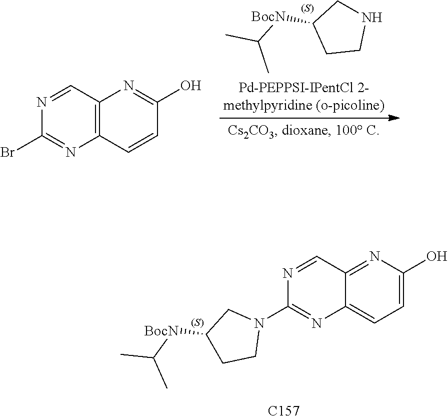

To a stirred solution of 2-bromopyrido[3,2-d]pyrimidin-6-ol (250 mg, 1.106 mmol, 1 equiv) and tert-butyl N-isopropyl-N-[(3S)-pyrrolidin-3-yl]carbamate (303 mg, 1.327 mmol, 1.2 equiv) in dioxane (10 mL) were added Pd-PEPPSI-IPentCl 2-methylpyridine (o-picoline) (47 mg, 0.055 mmol, 0.05 equiv) and Cs₂CO₃ (721 mg, 2.212 mmol, 2 equiv) in portions at room temperature under nitrogen atmosphere. The resulting mixture was stirred for 3 hr at 100° C. under nitrogen atmosphere. The mixture was allowed to cool down to room temperature. The resulting mixture was concentrated under reduced pressure. The residue was purified by silica gel column chromatography, eluted with PE/EA (1:10) to afford tert-butyl N-[(3S)-1-{6-hydroxypyrido[3,2-d]pyrimidin-2-yl}pyrrolidin-3-yl]-N-isopropylcarbamate (190 mg, 46%) as a solid. LCMS (ES, m/z): 374 [M−H]⁺

### Synthesis of Intermediate C158

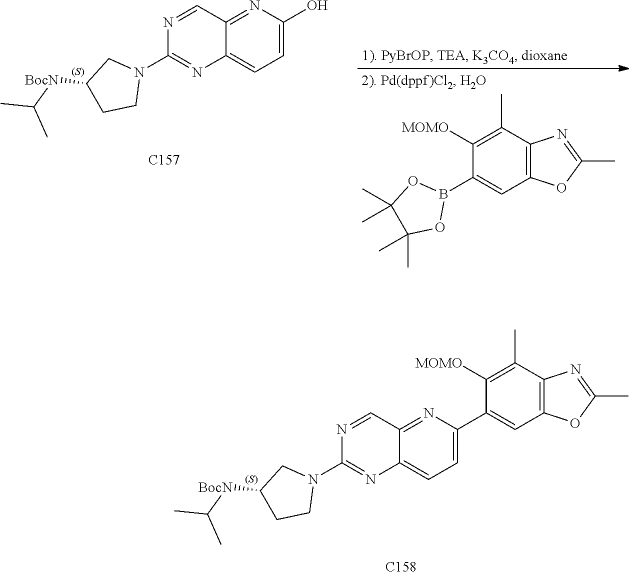

To a stirred solution of tert-butyl N-[(3S)-1-{6-hydroxypyrido[3,2-d]pyrimidin-2-yl}pyrrolidin-3-yl]-N-isopropylcarbamate (150 mg, 0.402 mmol, 1 equiv) and PyBrOP (314 mg, 0.603 mmol, 1.5 equiv) in dioxane (10 mL) were added TEA (122 mg, 1.206 mmol, 3 equiv) and K₂CO₃ (167 mg, 1.206 mmol, 3 equiv) in portions at room temperature under nitrogen atmosphere. The resulting mixture was stirred for 2 hr at 100° C. under nitrogen atmosphere. The mixture was allowed to cool down to room temperature. To the above mixture was added 5-(methoxymethoxy)-2,4-dimethyl-6-(4,4,5,5-tetramethyl-1,3,2-dioxaborolan-2-yl)-1,3-benzoxazole (268 mg, 0.804 mmol, 2 equiv), H₂O (0.2 mL) and Pd(dppf)Cl₂ (30 mg, 0.040 mmol, 0.1 equiv) in portions at room temperature. The resulting mixture was stirred for additional 3 hr at 100° C. The mixture was allowed to cool down to room temperature. The resulting mixture was concentrated under vacuum. The residue was purified by silica gel column chromatography, eluted with PE/EA (1:10) to afford tert-butyl N-isopropyl-N-[(3S)-1-{6-[5-(methoxymethoxy)-2,4-dimethyl-1,3-benzoxazol-6-yl]pyrido[3,2-d]pyrimidin-2-yl}pyrrolidin-3-yl]carbamate (100 mg, 44%) as a solid. LCMS (ES, m/z): 563 [M+H]⁺

### Synthesis of Compound 416

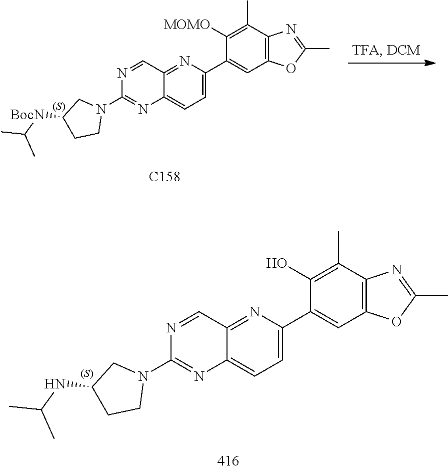

A solution of tert-butyl N-isopropyl-N-[(3S)-1-{6-[5-(methoxymethoxy)-2,4-dimethyl-1,3-benzoxazol-6-yl]pyrido[3,2-d]pyrimidin-2-yl}pyrrolidin-3-yl]carbamate (100 mg, 0.178 mmol, 1 equiv) in TFA (2 mL) and DCM (2 mL) was stirred for 2 hr at room temperature. The resulting mixture was concentrated under vacuum. The residue was basified to pH 8 with 7 M NH3 (g) in MeOH. The resulting mixture was concentrated under reduced pressure. The residue was purified by reverse flash chromatography (Condition 10, Gradient 3) to afford 6-{2-[(3S)-3-(isopropylamino)pyrrolidin-1-yl]pyrido[3,2-d]pyrimidin-6-yl}-2,4-dimethyl-1,3-benzoxazol-5-ol (35 mg, 47%) as a solid. LCMS (ES, m/z): 419 [M+H]^{+ 1}H NMR (300 MHz, DMSO-d₆) δ 14.34 (s, 1H), 9.38 (s, 1H), 8.61 (d, J=8.6 Hz, 1H), 8.30 (s, 1H), 8.10 (s, 1H), 3.86 (s, 3H), 3.62 (s, 2H), 2.96 (s, 1H), 2.63 (s, 3H), 2.41 (s, 3H), 1.90 (s, 1H), 1.24 (s, 1H), 1.12-1.04 (m, 6H).

### Example 168: Synthesis of Compounds 408-412

### Synthesis of Intermediate C159

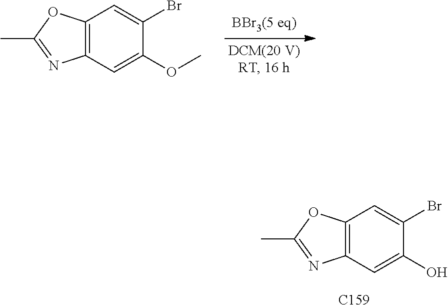

A solution of 6-bromo-5-methoxy-2-methyl-1,3-benzoxazole (12 g, 49.572 mmol, 1 equiv) and BBr₃ (62.09 g, 247.860 mmol, 5 equiv) in DCM (100 mL) was stirred for 16 hr at room temperature. The reaction was quenched by the addition of MeOH (5 mL) at 0° C. The resulting mixture was concentrated under reduced pressure. The mixture was basified to pH 8 with saturated NaHCO₃ (aq.). The aqueous layer was extracted with DCM (3×100 mL). The combined organic layers were washed with brine (1×100 mL), dried over anhydrous Na₂SO₄. After filtration, the filtrate was concentrated under reduced pressure. This resulted in 6-bromo-2-methyl-1,3-benzoxazol-5-ol (8 g, 70%) as a solid. LCMS (ES, m/z): 229 [M+H]⁺

### Synthesis of Intermediate C160

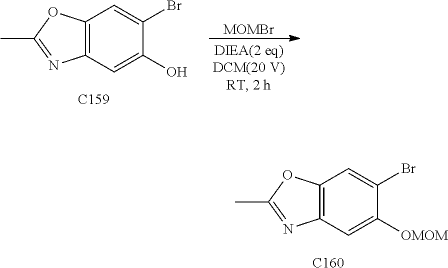

A solution of 6-bromo-2-methyl-1,3-benzoxazol-5-ol (8 g, 35.081 mmol, 1 equiv) and methane, bromomethoxy-(5.26 g, 42.097 mmol, 1.2 equiv) and DIEA (22.67 g, 175.405 mmol, 5 equiv) in DCM (50 mL) was stirred for 2 hr at room temperature. The reaction was quenched with H₂O at room temperature. The resulting mixture was extracted with DCM (3×50 mL). The combined organic layers were washed with NaCl Solution (1×50 mL), dried over anhydrous Na₂SO₄. After filtration, the filtrate was concentrated under reduced pressure. This resulted in 6-bromo-5-(methoxymethoxy)-2-methyl-1,3-benzoxazole (5 g, 52%) as a solid. LCMS (ES, m/z): 273 [M+H]⁺

### Synthesis of Intermediate C161

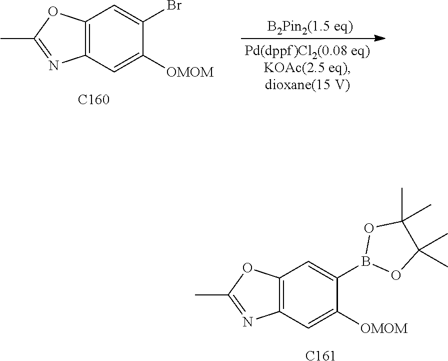

A solution of 6-bromo-5-(methoxymethoxy)-2-methyl-1,3-benzoxazole (5 g, 18.376 mmol, 1 equiv) in 1,4-dioxane (20 mL) was treated with potassium acetate (4.51 g, 45.940 mmol, 2.5 equiv) and Pd(dppf)Cl₂ (1.08 g, 1.470 mmol, 0.08 equiv) for 2 h at 80° C. under nitrogen atmosphere followed by the addition of 4,4,5,5-tetramethyl-2-(tetramethyl-1,3,2-dioxaborolan-2-yl)-1,3,2-dioxaborolane (7.00 g, 27.564 mmol, 1.5 equiv) dropwise portions at 80° C. The mixture was allowed to cool down to room temperature. The reaction was quenched with H₂O at room temperature. The resulting mixture was extracted with EA (3×20 mL). The combined organic layers were washed with NaCl Solution (2×20 mL), dried over anhydrous Na₂SO₄. After filtration, the filtrate was concentrated under reduced pressure. The residue was purified by silica gel column chromatography, eluted with PE:EA (3:1) to afford 5-(methoxymethoxy)-2-methyl-6-(4,4,5,5-tetramethyl-1,3,2-dioxaborolan-2-yl)-1,3-benzoxazole (3 g, 51%) as a solid. LCMS (ES, m/z): 320 [M+H]⁺

### Synthesis of Intermediate C162

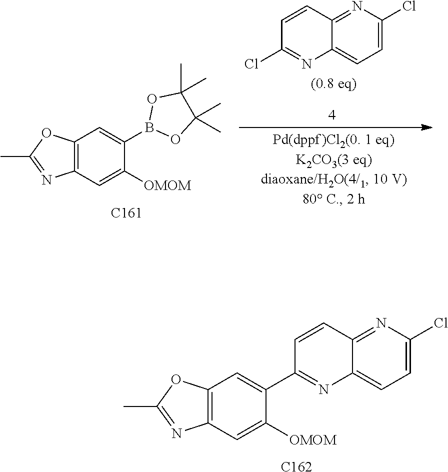

A solution of 2,6-dichloro-1,5-naphthyridine (1.5 g, 7.537 mmol, 1 equiv) in 1,4-dioxane (13 mL) and H₂O (4 mL) was treated with K₂CO₃ (3.12 g, 22.611 mmol, 3 equiv) and Pd(dppf)Cl₂CH₂Cl₂ (0.61 g, 0.754 mmol, 0.1 equiv) for 2 h at 80° C. under nitrogen atmosphere followed by the addition of 5-(methoxymethoxy)-2-methyl-6-(4,4,5,5-tetramethyl-1,3,2-dioxaborolan-2-yl)-1,3-benzoxazole (2.41 g, 7.537 mmol, 1 equiv) dropwise portions at 80° C. The mixture was allowed to cool down to room temperature. The reaction was quenched with H₂O at room temperature. The resulting mixture was extracted with EA (3×20 mL). The combined organic layers were washed with NaCl Solution (2×20 mL), dried over anhydrous Na₂SO₄. After filtration, the filtrate was concentrated under reduced pressure. The residue was purified by silica gel column chromatography, eluted with PE:EA (1:1) to afford 2-chloro-6-[5-(methoxymethoxy)-2-methyl-1,3-benzoxazol-6-yl]-1,5-naphthyridine (400 mg, 14%) as a solid. LCMS (ES, m/z): 356 [M+H]⁺

### Synthesis of Intermediate C163

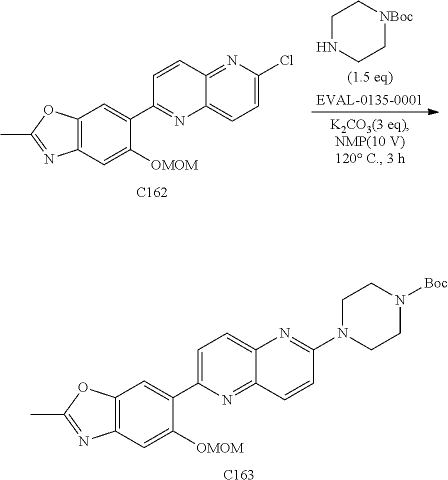

A solution of 2-chloro-6-[5-(methoxymethoxy)-2-methyl-1,3-benzoxazol-6-yl]-1,5-naphthyridine (80 mg, 0.225 mmol, 1 equiv) and tert-butyl piperazine-1-carboxylate (62.82 mg, 0.338 mmol, 1.5 equiv) and K₂CO₃ (93.23 mg, 0.675 mmol, 3 equiv) in NMP (2 mL) was stirred for 3 hr at 120° C. The mixture was allowed to cool down to room temperature. The reaction was quenched with H₂O at room temperature. The resulting mixture was extracted with EA (3×10 mL). The combined organic layers were washed with NaCl Solution (2×20 mL), dried over anhydrous Na₂SO₄. After filtration, the filtrate was concentrated under reduced pressure. This resulted in tert-butyl 4-{6-[5-(methoxymethoxy)-2-methyl-1,3-benzoxazol-6-yl]-1,5-naphthyridin-2-yl}piperazine-1-carboxylate (130 mg, 83%) as a solid. LCMS (ES, m/z): 506 [M+H]⁺

### Synthesis of Compound 408

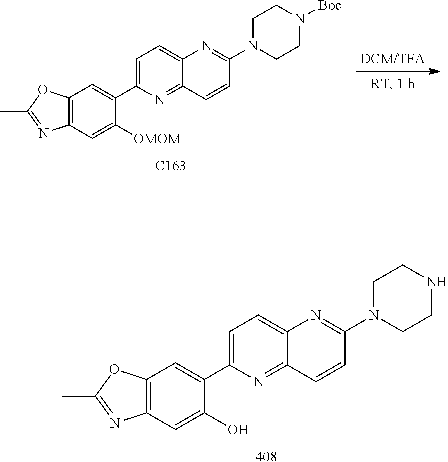

To a stirred solution of tert-butyl 4-{6-[5-(methoxymethoxy)-2-methyl-1,3-benzoxazol-6-yl]-1,5-naphthyridin-2-yl}piperazine-1-carboxylate (130 mg, 0.257 mmol, 1 equiv) in DCM (3 mL) was added TFA (1 mL) dropwise at room temperature. The mixture was stirred 1 h at room temperature. The reaction liquid was concentrated under reduced pressure. The crude product was purified by reverse phase flash chromatography to afford 2-methyl-6-[6-(piperazin-1-yl)-1,5-naphthyridin-2-yl]-1,3-benzoxazol-5-ol (11 mg, 12%) as a solid. LCMS (ES, m/z): 362 [M+H]⁺

### Analysis Data

| Compound Structure | Analysis data |
|---|---|
| 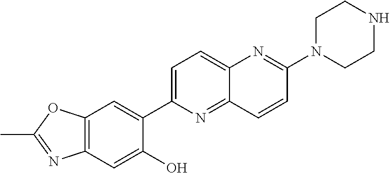 [SMILES: Cc1nc2cc(O)c(-c3ccc4nc(N5CCNCC5)ccc4n3)cc2o1] | LCMS: (ES, m/z): 362 [M + H] ⁺ ¹H NMR (400 MHz, Chloroform-d) δ 14.75 (s, 1H), 8.14-8.01 (m, 3H), 7.96 (s, 1H), 7.28 (s, 1H), 7.22 (d, J = 9.5 Hz, 1H), 3.85-3.78 (m, 4H), 3.10-3.03 (m, 4H), 2.65 (s, 3H). |
| 408 | |
| 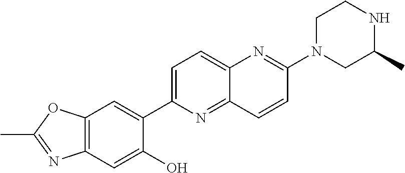 | LCMS: (ES, m/z): 376 [M + H] ⁺ ¹H NMR (400 MHz, Chloroform-d) δ 14.74 (s, 1H), 8.14-8.02 (m, 3H), 7.96 (s, 1H), 7.28 (s, 1H), 7.23 (d, J = 9.4 Hz, 1H), 4.47 (t, J = 11.1 Hz, 2H), 3.26 (d, J = 12.7 Hz, 2H), 3.23-3.14 (m, 2H), 3.07-2.97 (m, 1H), 2.83 (t, J = 11.6 Hz, 1H), 2.65 (s, 3H), 1.30 (d, J = 6.3 Hz, 3H). |
| 409 | |
| 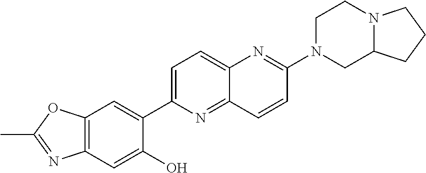 | LCMS: (ES, m/z): 389 [M + H] ⁺ ¹H NMR (400 MHz, Chloroform-d) δ 14.77 (s, 1H), 8.13-8.01 (m, 3H), 7.95 (s, 1H), 7.30 (m, 1H), 7.21 (m, 1H), 4.70 (d, J = 12.5 Hz, 1H), 4.54 (d, J = 12.7 Hz, 1H), 3.21 (s, 3H), 2.83 (s, 1H), 2.65 (s, 3H), 2.35 (s, 1H), 2.22 (s, 1H), 2.10 (s, 1H), 1.96 (s, 2H), 1.82 (s, 1H), 1.56 (s, 1H). |
| 410 | |
| 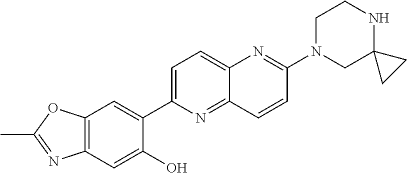 [SMILES: Cc1nc2cc(O)c(-c3ccc4nc(N5CCNC6(CC6)C5)ccc4n3)cc2o1] | LCMS: (ES, m/z): 389 [M + H] ⁺ ¹H NMR (400 MHz, Chloroform-d) δ 14.75 (s, 1H), 8.12-8.00 (m, 3H), 7.95 (s, 1H), 7.28 (s, 1H), 7.18 (d, J = 9.4 Hz, 1H), 3.83 (t, J = 5.2 Hz, 2H), 3.69 (s, 2H), 3.21- 3.14 (m, 2H), 2.65 (s, 3H), 0.78 (d, J = 8.5 Hz, 2H), 0.72 (s, 2H). |
| 411 | |
| 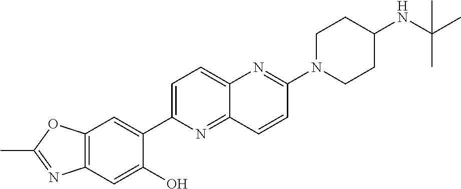 | LCMS: (ES, m/z): 389 [M + H] ⁺ ¹H NMR (400 MHz, DMSO-d₆) δ 14.48 (s, 1H), 8.44 (d, J = 7.6 Hz, 2H), 8.10 (dd, J = 17.9, 9.2 Hz, 2H), 7.54 (d, J = 9.5 Hz, 1H), 7.12 (s, 1H), 4.45 (d, J = 13.0 Hz, 2H), 3.14 (t, J = 12.4 Hz, 2H), 2.68 (m, 1H), 2.61 (s, 3H), 1.81 (s, 2H), 1.32-1.21 (m, 2H), 1.08 (s, 9H). |
| 412 | |

### Example 169: Synthesis of Compound 482

### Synthesis of Intermediate C164

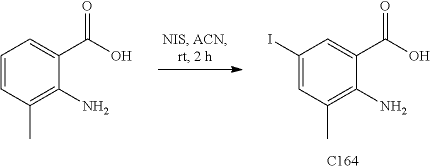

A solution of 2-amino-3-methylbenzoic acid (20 g, 132.306 mmol, 1.0 equiv) in acetonitrile (300 mL) was treated with NIS (35.72 g, 158.767 mmol, 1.2 equiv) at room temperature. The resulting mixture was stirred for 2 h at room temperature. A precipitate formed that was collected by filtration and washed with acetonitrile (2×50 mL). The resulting solid was dried under infrared light to afford 2-amino-5-iodo-3-methylbenzoic acid (30.2 g, 820%) as a solid. LCMS (ES, m/z): 278 [M+H]⁺.

### Synthesis of Intermediate C165

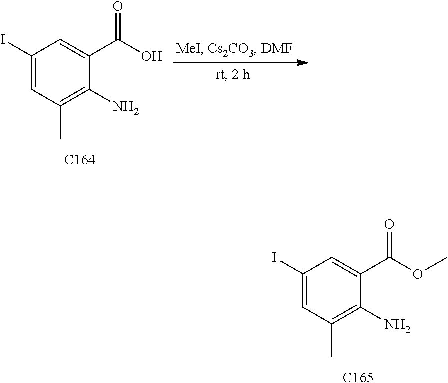

To a stirred mixture of 2-amino-5-iodo-3-methylbenzoic acid (30 g, 108.279 mmol, 1.0 equiv) and Cs₂CO₃ (70.56 g, 216.558 mmol, 2.0 equiv) in DMF (300 mL) was added Mel (19.98 g, 140.763 mmol, 1.3 equiv) at room temperature. The resulting mixture was stirred for 2 h at room temperature, then diluted with water (400 mL) and extracted with ethyl acetate (3×400 mL). The organic layers were combined, washed with brine (1×300 mL), dried over anhydrous Na₂SO₄, and filtered. The filtrate was concentrated under reduced pressure to afford methyl 2-amino-5-iodo-3-methylbenzoate (31 g, 98%) as a solid. LCMS (ES, m/z): 292 [M+H]⁺.

### Synthesis of Intermediate C166

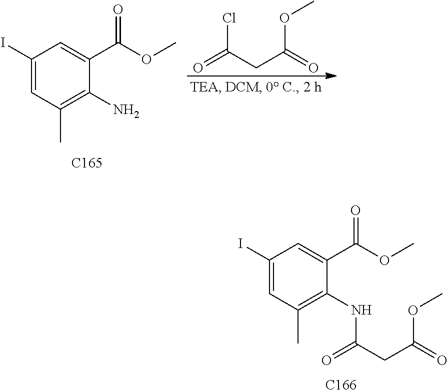

To a stirred mixture of methyl 2-amino-5-iodo-3-methylbenzoate (30 g, 103.062 mmol, 1.0 equiv) and TEA (20.86 g, 206.124 mmol, 2.0 equiv) in DCM (300 mL) was added methyl 3-chloro-3-oxopropanoate (15.48 g, 113.368 mmol, 1.1 equiv) dropwise at 0° C. under nitrogen atmosphere. The resulting mixture was stirred for 2 h at 0° C. under nitrogen atmosphere, then quenched with water/ice. The resulting mixture was extracted with CH₂Cl₂ (2×200 mL). The organic layers were combined, washed with brine (1×100 mL), dried over anhydrous Na₂SO₄, and filtered. The filtrate was concentrated under reduced pressure to give a residue. The residue was purified by silica gel column chromatography, eluted with PE/EA (2:1) to afford methyl 5-iodo-2-(3-methoxy-3-oxopropanamido)-3-methylbenzoate (16 g, 40%) as a solid. LCMS (ES, m/z): 392 [M+H]⁺.

### Synthesis of Intermediate C167

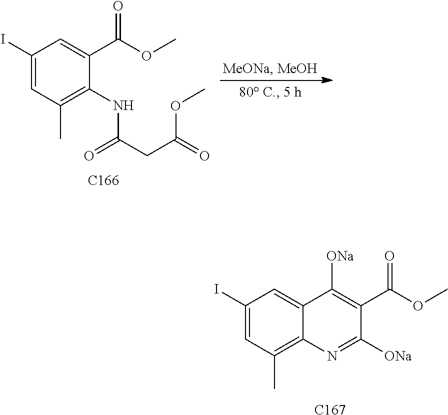

A solution of methyl 5-iodo-2-(3-methoxy-3-oxopropanamido)-3-methylbenzoate (15 g, 38.347 mmol, 1.0 equiv) in methanol (150 mL) was treated with MeONa (4.14 g, 76.694 mmol, 2.0 equiv) at room temperature. The resulting mixture was stirred for 5 h at 80° C. under nitrogen atmosphere, then cooled to room temperature. A precipitate formed that was collected by filtration and washed with methanol (1×20 mL). The resulting solid was dried under infrared light to afford methyl 6-iodo-8-methyl-2,4-bis(sodiooxy)quinoline-3-carboxylate (13.5 g, 87%) as a solid. LCMS (ES, m/z): 360 [M−2Na+3H]⁺.

### Synthesis of Intermediate C168

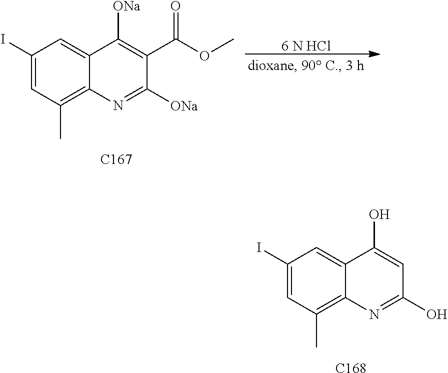

A mixture of methyl 6-iodo-8-methyl-2,4-bis(sodiooxy)quinoline-3-carboxylate (13 g, 34.732 mmol, 1.0 equiv) and HCl (6 M) (140 mL) in dioxane (140 mL) was stirred for 3 h at 90° C., then cooled to room temperature. A precipitate formed that was collected by filtration and washed with water (2×50 mL). The resulting solid was dried under infrared light to afford 6-iodo-8-methylquinoline-2,4-diol (8.1 g, 77%) as a solid. LCMS (ES, m/z): 302 [M+H]⁺.

### Synthesis of Intermediate C169

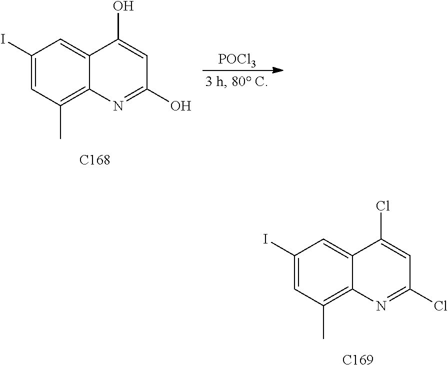

A mixture of 6-iodo-8-methylquinoline-2,4-diol (2 g, 6.643 mmol, 1.0 equiv) and phosphorus oxychloride (10 mL) was stirred for 3 h at 80° C. under nitrogen atmosphere, then cooled to room temperature. The resulting mixture was concentrated under reduced pressure to give a residue. The residue was purified by silica gel column chromatography, eluted with PE/EA (10:1) to afford 2,4-dichloro-6-iodo-8-methylquinoline (1.4 g, 62%) as a solid. LCMS (ES, m/z): 338 [M+H]⁺.

### Synthesis of Intermediate C170

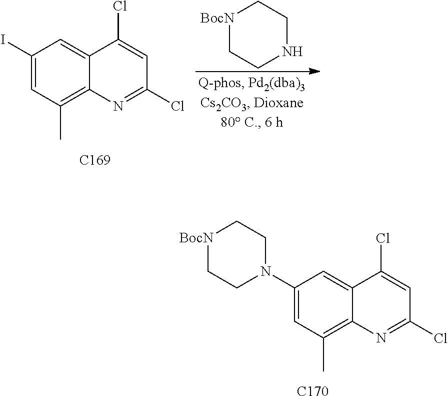 [SMILES: Cc1cc(I)cc2c(Cl)cc(Cl)nc12 ; CC(C)(C)OC(=O)N1CCNCC1 ; "C169" ; Cc1cc(N2CCN(C(=O)OC(C)(C)C)CC2)cc2c(Cl)cc(Cl)nc12 ; "C170"]

To a stirred mixture of 2,4-dichloro-6-iodo-8-methylquinoline (500 mg, 1.479 mmol, 1.0 equiv) and tert-butyl piperazine-1-carboxylate (330.6 mg, 1.775 mmol, 1.2 equiv) in dioxane (5 mL) was added Cs₂CO₃ (964.0 mg, 2.958 mmol, 2.0 equiv), 1,2,3,4,5-pentaphenyl-1′-(di-tert-butylphosphino)ferrocene (210.3 mg, 0.296 mmol, 0.2 equiv), and Pd₂(dba)₃ (135.5 mg, 0.148 mmol, 0.1 equiv) at room temperature under nitrogen atmosphere. The resulting mixture was stirred for 6 h at 80° C. under nitrogen atmosphere, then concentrated under reduced pressure to give a residue. The residue was purified by silica gel column chromatography, eluted with PE/EA (5:1) to afford tert-butyl 4-(2,4-dichloro-8-methylquinolin-6-yl)piperazine-1-carboxylate (268 mg, 46%) as a solid. LCMS (ES, m/z): 396 [M+H]⁺.

### Synthesis of Intermediate C171

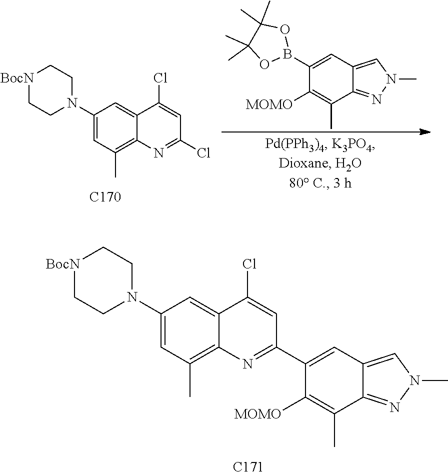

To a stirred mixture of tert-butyl 4-(2,4-dichloro-8-methylquinolin-6-yl)piperazine-1-carboxylate (160.0 mg, 0.404 mmol, 1.0 equiv) and 6-(methoxymethoxy)-2,7-dimethyl-5-(4,4,5,5-tetramethyl-1,3,2-dioxaborolan-2-yl) indazole (134.1 mg, 0.404 mmol, 1.0 equiv) in dioxane (2 mL) and water (0.4 mL) was added K₃PO₄ (257.0 mg, 1.212 mmol, 3.0 equiv) and Pd(PPh₃)₄ (46.6 mg, 0.040 mmol, 0.1 equiv) at room temperature under nitrogen atmosphere. The resulting mixture was stirred for 3 h at 80° C. under nitrogen atmosphere, then concentrated under reduced pressure to give a residue. The residue was purified by silica gel column chromatography, eluted with PE/EA (1:1) to afford tert-butyl 4-{4-chloro-2-[6-(methoxymethoxy)-2,7-dimethylindazol-5-yl]-8-methylquinolin-6-yl}piperazine-1-carboxylate (210 mg, 92%) as a solid. LCMS (ES, m/z): 566 [M+H]⁺.

### Synthesis of Compound 482

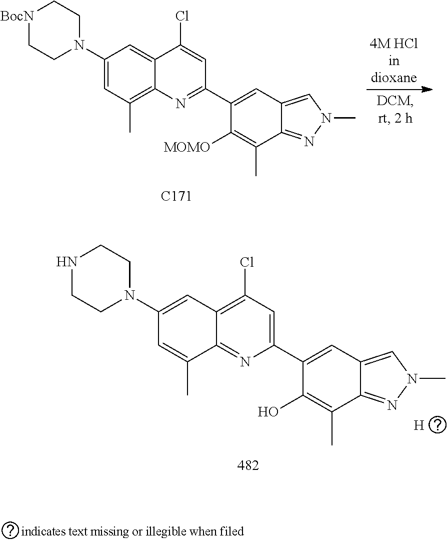

A solution of tert-butyl 4-{4-chloro-2-[6-(methoxymethoxy)-2,7-dimethylindazol-5-yl]-8-methylquinolin-6-yl}piperazine-1-carboxylate (100 mg, 0.177 mmol, 1.0 equiv) in DCM (1 mL) was treated with HCl (gas) in 1,4-dioxane (0.25 mL) at room temperature. The resulting mixture was stirred for 2 h at room temperature under nitrogen atmosphere, then concentrated under reduced pressure to give a residue. The residue was purified by Prep-HPLC (SunFire Prep C18 OBD Column 19*150 mm, 5 μm 10 nm, mobile phase, MeCN in water (0.05% HCl), 35% to 50% gradient in 15 min; detector, UV 254 nm) to afford 5-[4-chloro-8-methyl-6-(piperazin-1-yl)577yrrolidi-2-yl]-2,7-dimethylindazol-6-ol hydrochloride (26.5 mg, 33%) as a solid. LCMS (ES, m/z): 422 [M+H]⁺. ¹H NMR (300 MHz, DMSO-d₆) δ 14.60 (s, 1H), 9.46 (s, 2H), 8.58 (d, J=2.3 Hz, 2H), 8.43 (s, 1H), 7.73 (d, J=2.6 Hz, 1H), 7.20 (d, J=2.6 Hz, 1H), 4.17 (s, 3H), 3.63 (t, J=5.1 Hz, 4H), 3.29 (s, 4H), 2.70 (s, 3H), 2.39 (s, 3H).

### Example 170: Synthesis of Compound 778

### Synthesis of Intermediate C172

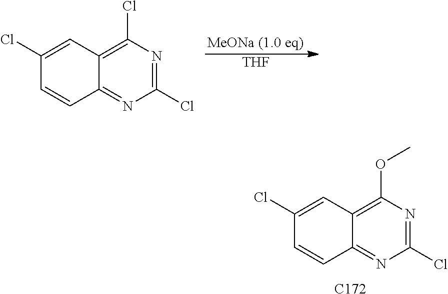

To a stirred solution of 2,4,6-trichloroquinazoline (300 mg, 1.285 mmol, 1 equiv) in tetrahydrofuran (3 mL) was added MeONa (231.39 mg, 1.285 mmol, 1.0 equiv, 30%) at room temperature. The resulting mixture was stirred for 2 h at 50° C., then concentrated under reduced pressure to give a residue. The residue was purified by silica gel column chromatography, eluted with PE/EA (3:1) to afford 2,6-dichloro-4-methoxyquinazoline (240 mg, 82%) as a solid. LCMS (ES, m/z): 229 [M+H]⁺.

### Synthesis of Intermediate C173

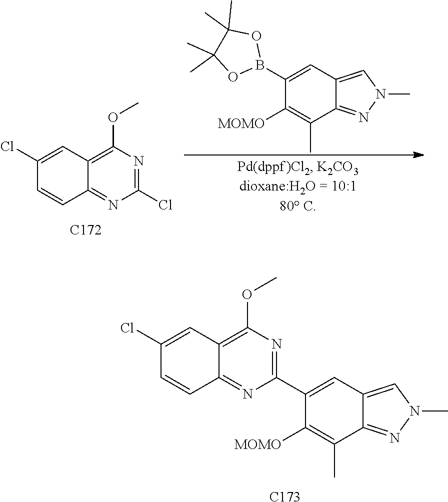

To a stirred mixture of 2,6-dichloro-4-methoxyquinazoline (240 mg, 1.048 mmol, 1 equiv) and 6-(methoxymethoxy)-2,7-dimethyl-5-(4,4,5,5-tetramethyl-1,3,2-dioxaborolan-2-yl) indazole (452.50 mg, 1.362 mmol, 1.3 equiv) in dioxane (3 mL) and water (0.6 mL) was added K₂CO₃ (434.42 mg, 3.144 mmol, 3 equiv) and Pd(dppf)Cl₂ (76.67 mg, 0.105 mmol, 0.1 equiv) at room temperature under nitrogen atmosphere. The resulting mixture was stirred for 4 h at 80° C. under nitrogen atmosphere, then concentrated under reduced pressure to give a residue. The residue was purified by silica gel column chromatography, eluted with PE/EA (1:1) to afford 6-chloro-4-methoxy-2-[6-(methoxymethoxy)-2,7-dimethylindazol-5-yl]quinazoline (160 mg, 38%) as an oil. LCMS (ES, m/z): 399 [M+H]⁺.

### Synthesis of Intermediate C174

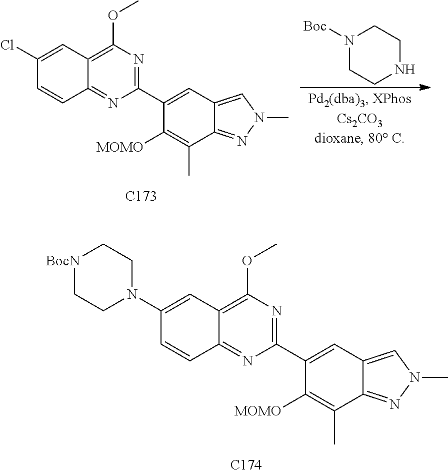

To a stirred mixture of 6-chloro-4-methoxy-2-[6-(methoxymethoxy)-2,7-dimethylindazol-5-yl]quinazoline (90 mg, 0.226 mmol, 1 equiv) and tert-butyl piperazine-1-carboxylate (50.43 mg, 0.271 mmol, 1.2 equiv) in dioxane (2 mL) was added Cs₂CO₃ (221.24 mg, 0.678 mmol, 3 equiv), Xphos (21.51 mg, 0.045 mmol, 0.2 equiv), and Pd₂(dba)₃ (20.66 mg, 0.023 mmol, 0.1 equiv) at room temperature under nitrogen atmosphere. The resulting mixture was stirred for 4 h at 80° C. under nitrogen atmosphere, then concentrated under reduced pressure to give a residue. The residue was purified by silica gel column chromatography, eluted with CH₂Cl₂/MeOH (20:1) to afford tert-butyl 4-{4-methoxy-2-[6-(methoxymethoxy)-2,7-dimethylindazol-5-yl]quinazolin-6-yl}piperazine-1-carboxylate (90 mg, 73%) as a solid. LCMS (ES, m/z): 549 [M+H]⁺.

### Synthesis of Compound 778

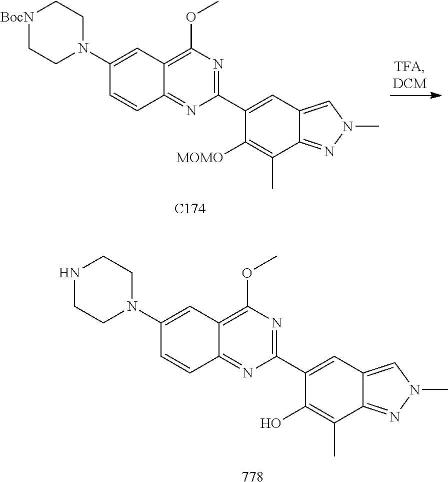

To a stirred solution of tert-butyl 4-{4-methoxy-2-[6-(methoxymethoxy)-2,7-dimethylindazol-5-yl]quinazolin-6-yl}piperazine-1-carboxylate (90 mg, 0.164 mmol, 1 equiv) in DCM (1 mL) was added TFA (1 mL) at room temperature. The resulting mixture was stirred for 1 h at room temperature, then concentrated under reduced pressure to give a residue. The residue was purified by Prep-HPLC (Condition 13, Gradient 1) to afford 5-[4-methoxy-6-(piperazin-1-yl)quinazolin-2-yl]-2,7-dimethylindazol-6-ol (21.5 mg, 32%) as a solid. LCMS (ES, m/z): 405 [M+H]⁺. ¹H NMR (400 MHz, DMSO-d₆) δ 13.96 (s, 1H), 8.86 (s, 1H), 8.40 (s, 1H), 7.87 (d, J=9.2 Hz, 1H), 7.80 (dd, J=9.3, 2.7 Hz, 1H), 7.31 (d, J=2.6 Hz, 1H), 4.30 (s, 3H), 4.15 (s, 3H), 3.30 (t, J=5.1 Hz, 4H), 2.97 (t, J=5.0 Hz, 4H), 2.39 (s, 3H).

### Example 171: Synthesis of Compound 502

### Synthesis of Intermediate C175

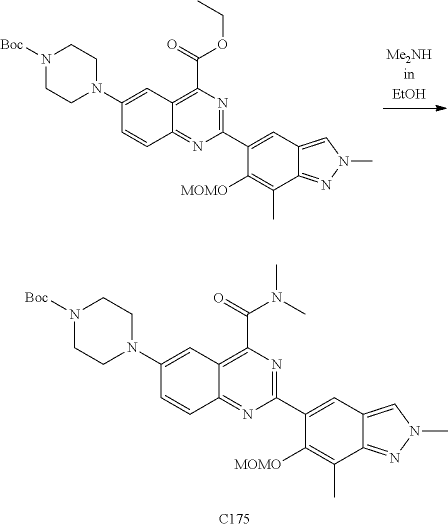

To a stirred solution of ethyl 6-[4-(tert-butoxycarbonyl)piperazin-1-yl]-2-[6-(methoxymethoxy)-2,7-dimethylindazol-5-yl]quinazoline-4-carboxylate (160 mg, 0.271 mmol, 1 equiv) in dimethylamine (2 M in ethanol) (8 mL) at room temperature. The resulting mixture was stirred overnight at 60° C., then concentrated under reduced pressure to afford tert-butyl 4-[4-(dimethylcarbamoyl)-2-[6-(methoxymethoxy)-2,7-dimethylindazol-5-yl]quinazolin-6-yl]piperazine-1-carboxylate (75 mg, 47%) as a solid. LCMS (ES, m/z): 590 [M+H]⁺.

### Synthesis of Compound 502

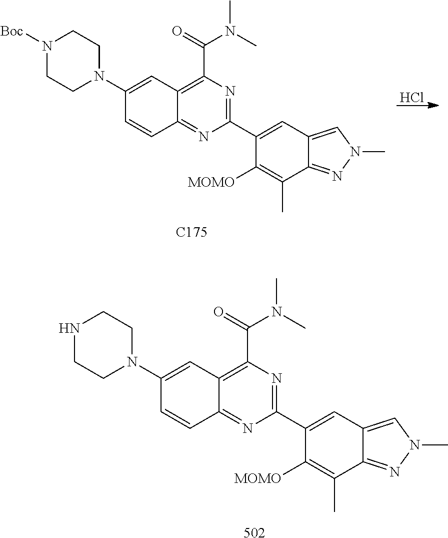

To a stirred solution of tert-butyl 4-[4-(dimethylcarbamoyl)-2-[6-(methoxymethoxy)-2,7-dimethylindazol-5-yl]quinazolin-6-yl]piperazine-1-carboxylate (75 mg, 0.127 mmol, 1 equiv) in dioxane (1 mL) was added HCl (gas) in 1,4-dioxane (1 mL) at room temperature. The resulting mixture was stirred for 1 h at room temperature, then concentrated under reduced pressure to give a residue. The residue was purified by Prep-HPLC (Condition 13, Gradient 2) to afford 2-(6-hydroxy-2,7-dimethylindazol-5-yl)-N,N-dimethyl-6-(piperazin-1-yl)quinazoline-4-carboxamide (20.2 mg, 36%) as a solid. LCMS (ES, m/z): 446 [M+H]⁺. ¹H NMR (400 MHz, DMSO-d₆) δ 13.37 (s, 1H), 8.83 (s, 1H), 8.40 (s, 1H), 8.04-7.93 (m, 2H), 6.91 (d, J=2.5 Hz, 1H), 4.15 (s, 3H), 3.25 (t, J=5.0 Hz, 4H), 3.20 (s, 3H), 2.90 (d, J=9.1 Hz, 7H), 2.40 (s, 3H).

### Example 172: Synthesis of Compound 503

### Synthesis of Intermediate C176

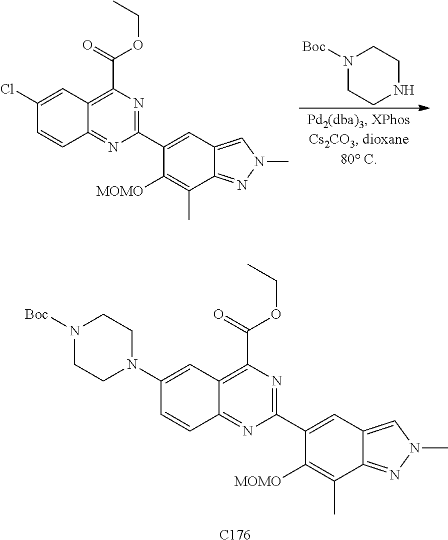

To a stirred mixture of ethyl 6-chloro-2-[6-(methoxymethoxy)-2,7-dimethylindazol-5-yl]quinazoline-4-carboxylate (200 mg, 0.454 mmol, 1 equiv) and tert-butyl piperazine-1-carboxylate (92.94 mg, 0.499 mmol, 1.1 equiv) in dioxane (4 mL) was added Cs₂CO₃ (221.71 mg, 0.681 mmol, 1.5 equiv), Xphos (43.25 mg, 0.091 mmol, 0.2 equiv), and Pd₂(dba)₃ (41.54 mg, 0.045 mmol, 0.1 equiv) at room temperature under nitrogen atmosphere. The resulting mixture was stirred for 4 h at 80° C. under nitrogen atmosphere, then concentrated under reduced pressure to give a residue. The residue was purified by silica gel column chromatography, eluted with CH₂Cl₂/MeOH (20:1) to afford ethyl 6-[4-(tert-butoxycarbonyl)piperazin-1-yl]-2-[6-(methoxymethoxy)-2,7-dimethylindazol-5-yl]quinazoline-4-carboxylate (170 mg, 63%) as a solid. LCMS (ES, m/z): 591 [M+H]⁺.

### Synthesis of Intermediate C177

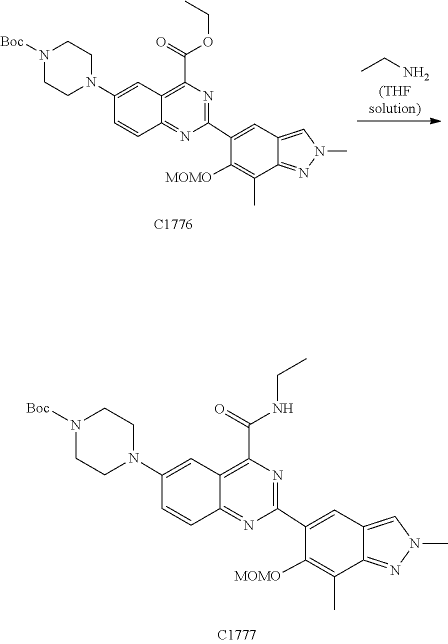

A mixture of ethyl 6-[4-(tert-butoxycarbonyl)piperazin-1-yl]-2-[6-(methoxymethoxy)-2,7-dimethylindazol-5-yl]quinazoline-4-carboxylate (80 mg, 0.135 mmol, 1 equiv) in ethylamine solution (2.0 M in THF 8 mL) stirred overnight at 80° C. The resulting mixture was concentrated under reduced pressure to afford tert-butyl 4-[4-(ethylcarbamoyl)-2-[6-(methoxymethoxy)-2,7-dimethylindazol-5-yl]quinazolin-6-yl]piperazine-1-carboxylate (75 mg, 94%) as a solid. LCMS (ES, m/z): 590 [M+H]⁺.

### Synthesis of Compound 503

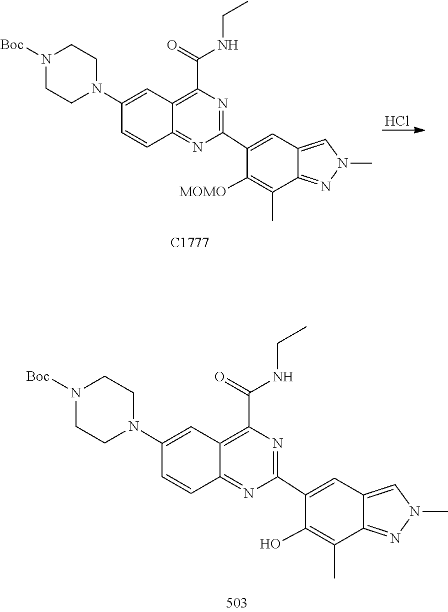

To a stirred solution of tert-butyl 4-[4-(ethylcarbamoyl)-2-[6-(methoxymethoxy)-2,7-dimethylindazol-5-yl]quinazolin-6-yl]piperazine-1-carboxylate (75 mg, 0.127 mmol, 1 equiv) in dioxane (1 mL) was added HCl (gas) in 1,4-dioxane (1 mL) at room temperature. The resulting mixture was stirred for 1 h at room temperature, then concentrated under reduced pressure to give a residue. The residue was purified by Prep-HPLC (Condition 13, Gradient 3) to afford N-ethyl-2-(6-hydroxy-2,7-dimethylindazol-5-yl)-6-(piperazin-1-yl)quinazoline-4-carboxamide (15.5 mg, 27%) as a solid. LCMS (ES, m/z): 446 [M+H]⁺. ¹H NMR (400 MHz, Methanol-d₄) δ 8.95 (d, J=0.8 Hz, 1H), 8.21-8.15 (m, 2H), 7.85 (d, J=1.5 Hz, 2H), 4.18 (s, 3H), 3.60 (q, J=7.2 Hz, 2H), 3.38 (t, J=5.1 Hz, 4H), 3.07 (t, J=5.1 Hz, 4H), 2.47 (s, 3H), 1.39 (t, J=7.3 Hz, 3H).

### Example 173: Synthesis of Compound 484

### Synthesis of Intermediate C178

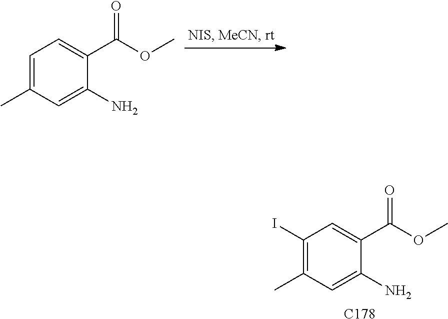

To a stirred solution of methyl 2-amino-4-methylbenzoate (25.0 g, 151.339 mmol, 1.0 equiv) in acetonitrile (500 mL) was added NIS (51.0 g, 227.008 mmol, 1.5 equiv) in portions at room temperature. The resulting mixture was stirred for 3 h at room temperature, then diluted with water (500 mL) and extracted with ethyl acetate (2×500 mL). The organic layers were combined, washed with brine (1×500 mL), dried over anhydrous Na₂SO₄, and filtered. The filtrate was concentrated under reduced pressure to give a residue. The residue was purified by silica gel column chromatography, eluted with PE/EA (5:1) to afford methyl 2-amino-5-iodo-4-methylbenzoate (35 g, 72%) as a solid. LCMS (ES, m/z): 292 [M+H]⁺.

### Synthesis of Intermediate C179

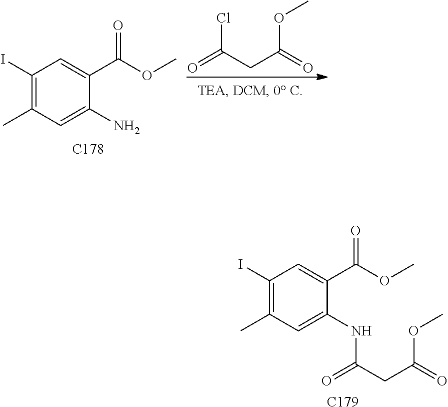

To a stirred mixture of methyl 2-amino-5-iodo-4-methylbenzoate (30.0 g, 103.062 mmol, 1.0 equiv) and TEA (20.8 g, 206.124 mmol, 2.0 equiv) in DCM (500 mL) was added methyl 3-chloro-3-oxopropanoate (15.4 g, 113.368 mmol, 1.1 equiv) dropwise at 0° C. The resulting mixture was stirred for 2 h at 0° C., then diluted with water (500 mL) and extracted with CH₂Cl₂ (2×500 mL). The organic layers were combined, washed with brine (1×500 mL), dried over anhydrous Na₂SO₄, and filtered. The filtrate was concentrated under reduced pressure to give a residue. The residue was purified by silica gel column chromatography, eluted with PE/EA (5:1) to afford methyl 5-iodo-2-(3-methoxy-3-oxopropanamido)-4-methylbenzoate (32 g, 72%) as a solid. LCMS (ES, m/z): 392 [M+H]⁺.

### Synthesis of Intermediate C180

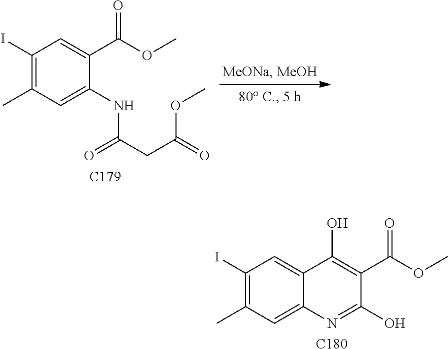

A mixture of methyl 5-iodo-2-(3-methoxy-3-oxopropanamido)-4-methylbenzoate (31.0 g, 79.251 mmol, 1.0 equiv), methanol (600 mL), and MeONa (8.5 g, 158.502 mmol, 2.0 equiv) was stirred for 5 h at 80° C. A precipitate formed that was collected by filtration and washed with methanol (1×100 mL) to afford methyl 6-iodo-7-methyl-2,4-bis(sodiooxy)quinoline-3-carboxylate (30 g, 85%) as a solid. LCMS (ES, m/z): 392 [M+H]⁺.

### Synthesis of Intermediate C181

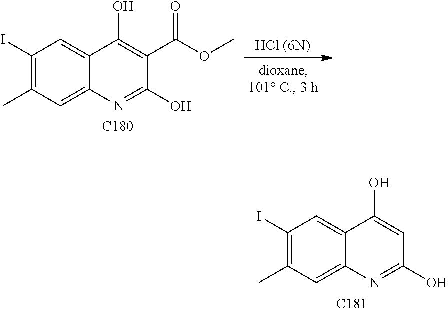 [SMILES: COC(=O)c1c(O)nc2cc(C)c(I)cc2c1O ; "C180" ; Cc1cc2nc(O)cc(O)c2cc1I ; "C181"]

To a stirred solution of methyl 6-iodo-7-methyl-2,4-bis(sodiooxy)quinoline-3-carboxylate (28.0 g, 69.465 mmol, 1.0 equiv) in dioxane (200 mL) was added HCl (200 mL) dropwise at room temperature. The resulting mixture was stirred for 3 h at 110° C. A precipitate formed that was collected by filtration and washed with 1,4-dioxane (1×50 mL) to 584yrrol 6-iodo-7-methylquinoline-2,4-diol (18 g, 80%) as a solid. LCMS (ES, m/z): 302 [M+H]⁺.

### Synthesis of Intermediate C182

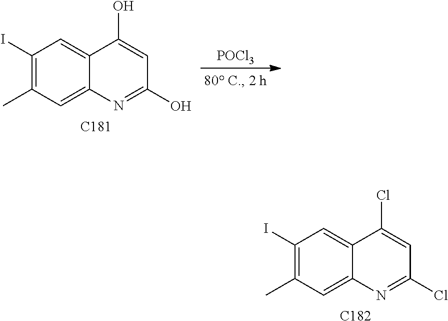

A mixture of 6-iodo-7-methylquinoline-2,4-diol (10.0 g, 33.213 mmol, 1.0 equiv) and phosphorus oxychloride (30 mL) was stirred for 2 h at 80° C. The resulting mixture was concentrated under reduced pressure, diluted with water (200 mL), and extracted with ethyl acetate (2×200 mL). The organic layers were combined, washed with brine (2×200 mL), dried over anhydrous Na₂SO₄, and filtered. The filtrate was concentrated under reduced pressure to give a residue. The residue was purified by silica gel column chromatography, eluted with PE/EA (1:1) to afford 2,4-dichloro-6-iodo-7-methylquinoline (5 g, 41%) as a solid. LCMS (ES, m/z): 337 [M+H]⁺.

### Synthesis of Intermediate C183

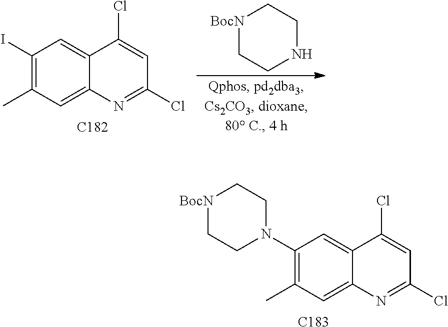 [SMILES: Cc1cc2nc(Cl)cc(Cl)c2cc1I ; CC(C)(C)OC(=O)N1CCNCC1 ; "C182" ; Cc1cc2nc(Cl)cc(Cl)c2cc1N1CCN(C(=O)OC(C)(C)C)CC1 ; "C183"]

To a solution of 2,4-dichloro-6-iodo-7-methylquinoline (600.0 mg, 1.775 mmol, 1.0 equiv) and tert-butyl piperazine-1-carboxylate (495.9 mg, 2.662 mmol, 1.5 equiv) in dioxane (12 mL) was added Cs₂CO₃ (1156.8 mg, 3.550 mmol, 2.0 equiv), Pd₂(dba)₃ (162.5 mg, 0.177 mmol, 0.1 equiv), and 1,2,3,4,5-pentaphenyl-1′-(di-tert-butylphosphino)ferrocene (378.5 mg, 0.532 mmol, 0.3 equiv). The reaction mixture was stirred overnight at 80° C. under a nitrogen atmosphere, then concentrated under reduced pressure to give a residue. The residue was purified by silica gel column chromatography, eluted with PE/EA (1:1) to afford tert-butyl 4-(2,4-dichloro-7-methylquinolin-6-yl) piperazine-1-carboxylate (400 mg, 52%) as a solid. LCMS (ES, m/z): 396 [M+H]⁺.

### Synthesis of Intermediate C184

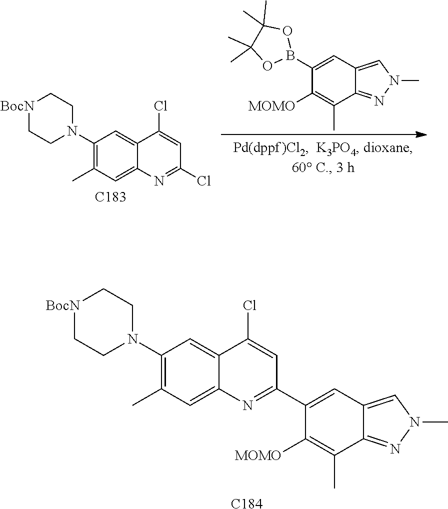

To a mixture of tert-butyl 4-(2,4-dichloro-7-methylquinolin-6-yl)piperazine-1-carboxylate (140.0 mg, 0.353 mmol, 1.0 equiv) and 6-(methoxymethoxy)-2,7-dimethyl-5-(4,4,5,5-tetramethyl-1,3,2-dioxaborolan-2-yl) indazole (117.3 mg, 0.353 mmol, 1.0 equiv) in dioxane (4 mL) and water (1 mL) was added K₃PO₄ (149.9 mg, 0.706 mmol, 2.0 equiv) and Pd(dppf)Cl₂ (25.8 mg, 0.035 mmol, 0.1 equiv). The reaction mixture was stirred for 3 h at 60° C. under a nitrogen atmosphere, then concentrated under reduced pressure to give a residue. The residue was purified by silica gel column chromatography, eluted with PE/EA (3:1) to afford tert-butyl 4-{4-chloro-2-[6-(methoxymethoxy)-2,7-dimethylindazol-5-yl]-7-methylquinolin-6-yl}piperazine-1-carboxylate (60 mg, 28%) as a solid. LCMS (ES, m/z): 566 [M+H]⁺.

### Synthesis of Compound 484

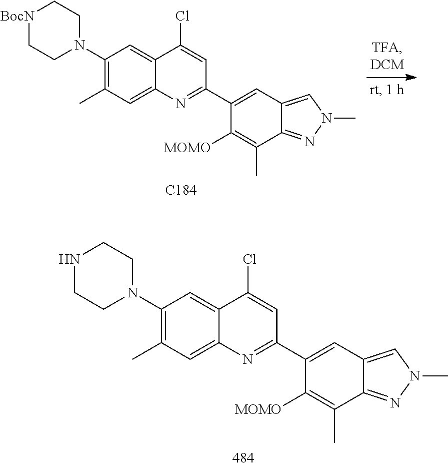

To a stirred solution of tert-butyl 4-{4-chloro-2-[6-(methoxymethoxy)-2,7-dimethylindazol-5-yl]-7-methylquinolin-6-yl}piperazine-1-carboxylate (60.0 mg, 0.106 mmol, 1.0 equiv) in DCM (1 mL) was added TFA (0.5 mL) dropwise at room temperature. The resulting mixture was stirred for 1 h at room temperature, then concentrated under reduced pressure to give a residue. The residue was purified by Prep-HPLC (SunFire Prep Column 19*150 mm, 10 nm, mobile phase, MeCN in water (0.05% NH40H), 30% to 50% gradient in 7 min; detector, UV 254 nm) to afford 5-[4-chloro-7-methyl-6-(piperazin-1-yl)586yrrolidi-2-yl]-2,7-dimethylindazol-6-ol (15.5 mg, 34%) as a solid. LCMS (ES, m/z): 422 [M+H]⁺. ¹H NMR (400 MHz, DMSO-d₆) δ 14.26 (s, 1H), 8.57 (d, J=9.8 Hz, 2H), 8.38 (s, 1H), 7.96 (s, 1H), 7.54 (s, 1H), 4.15 (s, 3H), 2.96 (d, J=4.3 Hz, 8H), 2.50 (s, 3H), 2.39 (s, 3H).

### Example 174: Synthesis of Compound 486

### Synthesis of Intermediate C185

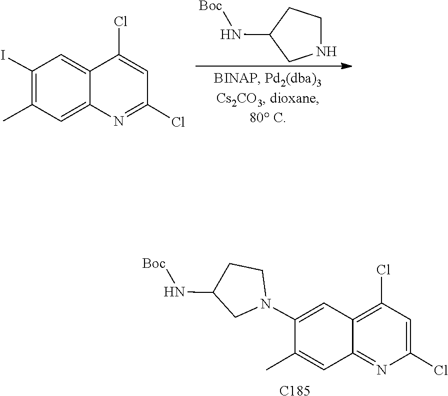 [SMILES: Cc1cc2nc(Cl)cc(Cl)c2cc1I ; CC(C)(C)OC(=O)NC1CCNC1 ; Cc1cc2nc(Cl)cc(Cl)c2cc1N1CCC(NC(=O)OC(C)(C)C)C1 ; "C185"]

To a mixture of 2,4-dichloro-6-iodo-7-methylquinoline (1.3 g, 3.846 mmol, 1.0 equiv) and tert-butyl N-(586yrrolidine-3-yl)carbamate (0.7 g, 3.846 mmol, 1.0 equiv) in dioxane (20 mL) was added t-BuONa (0.74 g, 7.692 mmol, 2.0 equiv), Xantphos (444.5 mg, 0.769 mmol, 0.2 equiv), and Pd₂(dba)₃ (352.6 mg, 0.385 mmol, 0.1 equiv). The reaction mixture was stirred for 5 h at 80° C. under a nitrogen atmosphere, then concentrated under reduced pressure to give a residue. The residue was purified by silica gel column chromatography, eluted with PE/EA (5:1) to afford tert-butyl N-[1-(2,4-dichloro-7-methylquinolin-6-yl)587yrrolidine-3-yl]carbamate (600 mg, 35%) as a solid. LCMS (ES, m/z): 396 [M+H]⁺.

### Synthesis of Intermediate C186

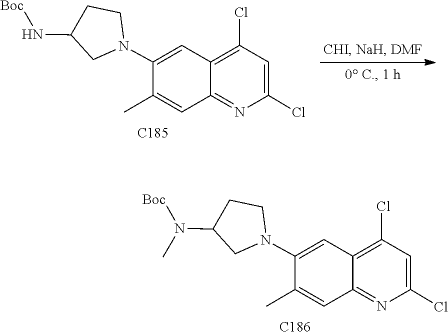 [SMILES: Cc1cc2nc(Cl)cc(Cl)c2cc1N1CCC(NC(=O)OC(C)(C)C)C1 ; "C185" ; Cc1cc2nc(Cl)cc(Cl)c2cc1N1CCC(N(C)C(=O)OC(C)(C)C)C1 ; "C186"]

To a stirred mixture of tert-butyl N-[1-(2,4-dichloro-7-methylquinolin-6-yl)587yrrolidine-3-yl]carbamate (580.0 mg, 1.464 mmol, 1 equiv) and NaH (70.2 mg, 2.928 mmol, 2.0 equiv) in DMF (10 mL) was added CH₃I (415.4 mg, 2.928 mmol, 2.0 equiv) dropwise at room temperature. The resulting mixture was stirred for 1 h at 0° C., then quenched with saturated NH₄Cl (aq.) (50 mL) at 0° C. and extracted with ethyl acetate (2×50 mL). The organic layers were combined, washed with water (1×50 mL), brine (1×50 mL), dried over anhydrous Na₂SO₄, and filtered. The filtrate was concentrated under reduced pressure to afford tert-butyl N-[1-(2,4-dichloro-7-methylquinolin-6-yl) 587yrrolidine-3-yl]-N-methylcarbamate (450 mg, 64%) as a solid. LCMS (ES, m/z): 410 [M+H]⁺.

### Synthesis of Intermediate C187

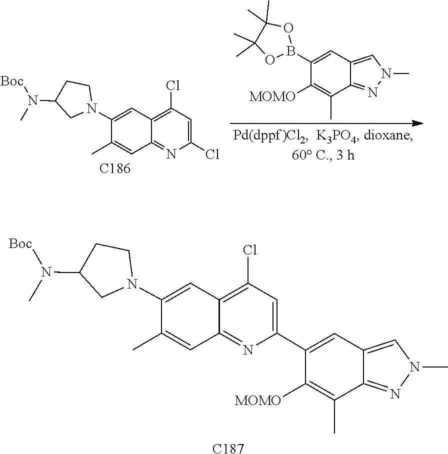

To a mixture of tert-butyl N-[1-(2,4-dichloro-7-methylquinolin-6-yl)587yrrolidine-3-yl]-N-methylcarbamate (200.0 mg, 0.487 mmol, 1.0 equiv) and 6-(methoxymethoxy)-2,7-dimethyl-5-(4,4,5,5-tetramethyl-1,3,2-587yrrolidine587e-2-yl) indazole (161.9 mg, 0.487 mmol, 1 equiv) in dioxane (4 mL) and water (1 mL) was added K₃PO₄ (206.9 mg, 0.974 mmol, 2 equiv) and Pd(PPh₃)₄(56.3 mg, 0.049 mmol, 0.1 equiv). The reaction mixture was stirred for 3 h at 60° C. under a nitrogen atmosphere, then concentrated under reduced pressure to give a residue. The residue was purified by silica gel column chromatography, eluted with PE/EA (1:1) to afford tert-butyl N-(1-{4-chloro-2-[6-(methoxymethoxy)-2,7-dimethylindazol-5-yl]-7-methylquinolin-6-yl}588yrrolidine-3-yl)-N-methylcarbamate (150 mg, 48%) as a solid. LCMS (ES, m/z): 580 [M+H]⁺.

### Synthesis of Compound 486

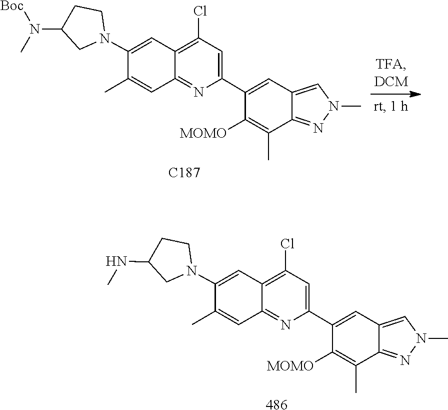

To a stirred solution of tert-butyl N-(1-{4-chloro-2-[6-(methoxymethoxy)-2,7-dimethylindazol-5-yl]-7-methylquinolin-6-yl}588yrrolidine-3-yl)-N-methylcarbamate (200.0 mg, 0.345 mmol, 1.0 equiv) in DCM (2 mL) was added TFA (0.5 mL) dropwise at room temperature. The resulting mixture was stirred for 1 h at room temperature, then concentrated under reduced pressure to give a residue. The residue was purified by Prep-HPLC (Condition 14, Gradient 1) to afford 5-{4-chloro-7-methyl-6-[3-(methylamino)pyrrolidin-1-yl]quinolin-2-yl}-2,7-dimethylindazol-6-ol (40 mg, 26%) as a solid. LCMS (ES, m/z): 436 [M+H]⁺. ¹H NMR (400 MHz, DMSO-d₆) δ 14.34 (s, 1H), 8.50 (s, 1H), 8.46 (s, 1H), 8.35 (s, 1H), 7.85 (d, J=1.0 Hz, 1H), 7.16 (s, 1H), 4.15 (s, 3H), 3.60 (dd, J=9.4, 5.9 Hz, 1H), 3.58-3.48 (m, 1H), 3.47-3.37 (m, 1H), 3.23 (dd, J=9.4, 4.6 Hz, 1H), 2.58 (s, 3H), 2.38 (s, 3H), 2.35 (s, 3H), 2.13 (dq, J=13.0, 6.6 Hz, 1H), 1.83 (dq, J=12.6, 6.4 Hz, 1H).

### Example 175: Synthesis of Compound 530

### Synthesis of Intermediate C188

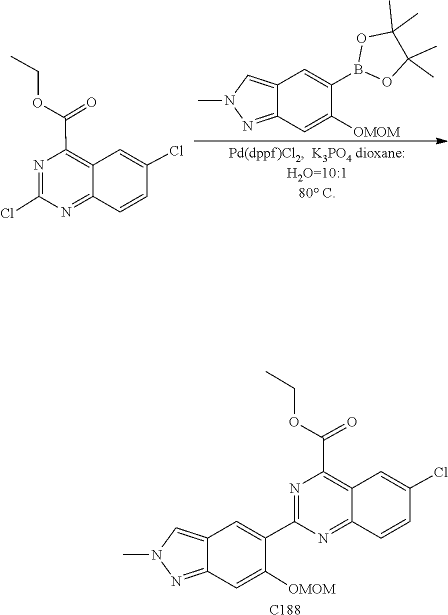

A mixture of ethyl 2,6-dichloroquinazoline-4-carboxylate (500 mg, 1.844 mmol, 1 equiv), 6-(methoxymethoxy)-2-methyl-5-(4,4,5,5-tetramethyl-1,3,2-dioxaborolan-2-yl) indazole (942.2 mg, 2.961 mmol, 1.61 equiv), Pd(PPh₃)₄(214 mg, 0.185 mmol, 0.10 equiv), and K₃PO₄ (783 mg, 3.689 mmol, 2.00 equiv) in dioxane (20 mL, 236.079 mmol, 128.00 equiv) and water (0.2 mL, 11.102 mmol, 6.02 equiv) was stirred for 2 h at 80° C. under nitrogen atmosphere. The resulting mixture was concentrated under reduced pressure to give a residue. The residue was purified by silica gel column chromatography, eluted with PE/EA (1:1) to afford ethyl 6-chloro-2-[6-(methoxymethoxy)-2-methylindazol-5-yl]quinazoline-4-carboxylate (550 mg, 70%) as a solid. LCMS (ES, m/z): 427 [M+H]⁺.

### Synthesis of Intermediate C189

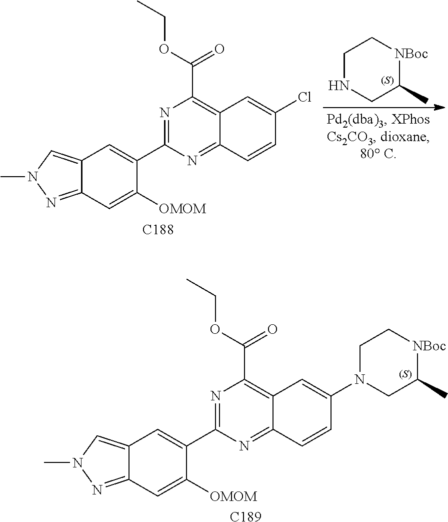

A mixture of ethyl 6-chloro-2-[6-(methoxymethoxy)-2-methylindazol-5-yl]quinazoline-4-carboxylate (250 mg, 0.586 mmol, 1 equiv), tert-butyl (2S)-2-methylpiperazine-1-carboxylate (176 mg, 0.879 mmol, 1.50 equiv), Pd₂(dba)₃ (53.7 mg, 0.059 mmol, 0.10 equiv), RuPhos (54.8 mg, 0.117 mmol, 0.20 equiv), and Cs₂CO₃ (382.6 mg, 1.174 mmol, 2.00 equiv) in dioxane (5 mL) was stirred for 2 h at 80° C. under nitrogen atmosphere, then cooled to room temperature. The resulting mixture was concentrated under reduced pressure to give a residue. The residue was purified by silica gel column chromatography, eluted with PE/EA (1:1) to afford ethyl 6-[(3S)-4-(tert-butoxycarbonyl)-3-methylpiperazin-1-yl]-2-[6-(methoxymethoxy)-2-methylindazol-5-yl]quinazoline-4-carboxylate (300 mg, 87%) as a solid. LCMS (ES, m/z): 591 [M+H]⁺.

### Synthesis of Intermediate C190

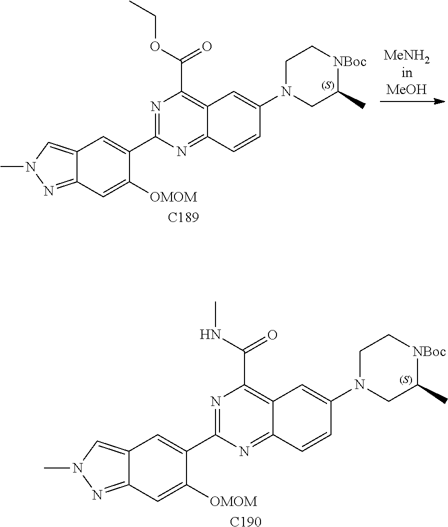

A mixture of 2M CH₃NH₂ solution in methanol (0.5 mL) and ethyl 6-[(3S)-4-(tert-butoxycarbonyl)-3-methylpiperazin-1-yl]-2-[6-(methoxymethoxy)-2-methylindazol-5-yl]quinazoline-4-carboxylate (50 mg, 0.085 mmol, 1 equiv) was stirred for 1 h at 80° C. The resulting mixture was concentrated under reduced pressure to afford tert-butyl (S)-4-(2-(6-(methoxymethoxy)-2-methyl-2H-indazol-5-yl)-4-(methylcarbamoyl)quinazolin-6-yl)-2-methylpiperazine-1-carboxylate (45 mg) as an oil. LCMS (ES, m/z): 276 [M+H]⁺.

### Synthesis of Compound 530

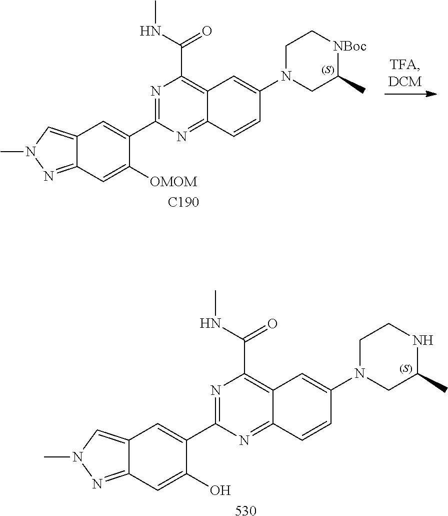

A solution of TFA (0.15 mL) in DCM (0.5 mL) was treated with tert-butyl (2S)-4-{2-[6-(methoxymethoxy)-2-methylindazol-5-yl]-4-(methylcarbamoyl)quinazolin-6-yl}-2-methylpiperazine-1-carboxylate (50 mg, 0.087 mmol, 1.00 equiv). The reaction mixture was stirred for 15 min at room temperature, then concentrated under reduced pressure to give a residue. The residue was purified by reverse phase flash chromatography (Condition 10, Gradient 8) to afford 2-(6-hydroxy-2-methylindazol-5-yl)-N-methyl-6-[(3S)-3-methylpiperazin-1-yl]quinazoline-4-carboxamide (18.1 mg, 48%) as a solid. LCMS (ES, m/z): 432 [M+H]⁺. ¹H NMR (400 MHz, DMSO-d₆) δ 13.21 (s, 1H), 9.23 (s, 2H), 8.45 (s, 1H), 8.16 (s, 1H), 7.99 (s, 2H), 6.91 (s, 1H), 4.14 (s, 3H), 3.78 (t, J=12.3 Hz, 2H), 3.10 (d, J=11.5 Hz, 1H), 3.07-2.75 (m, 6H), 1.12 (d, J=6.3 Hz, 3H).

### Example 176: Synthesis of Compound 532

### Synthesis of Intermediate C191

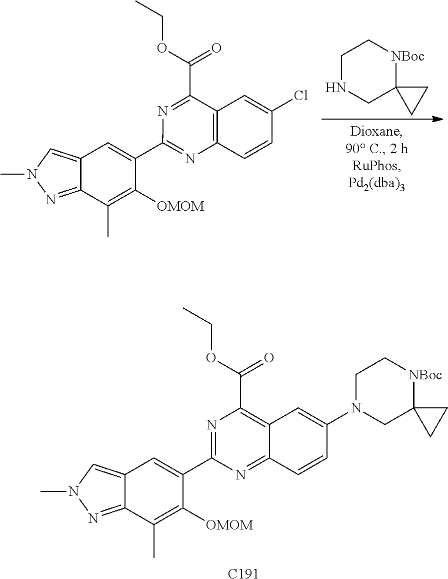

To a mixture of ethyl 6-chloro-2-[6-(methoxymethoxy)-2,7-dimethylindazol-5-yl]quinazoline-4-carboxylate (200 mg, 0.454 mmol, 1 equiv) and tert-butyl 4,7-diazaspiro[2.5]octane-4-carboxylate (144 mg, 0.681 mmol, 1.5 equiv) in dioxane (4 mL) was added Cs₂CO₃ (443 mg, 1.362 mmol, 3.0 equiv), RuPhos (42.3 mg, 0.091 mmol, 0.2 equiv), and Pd₂(dba)₃ (41 mg, 0.045 mmol, 0.1 equiv). The reaction mixture was stirred for 1 h at 90° C. under a nitrogen atmosphere, then concentrated under reduced pressure to give a residue. The residue was purified by reverse-phase flash chromatography (Condition 10, Gradient 8) to afford ethyl 6-[4-(tert-butoxycarbonyl)-4,7-diazaspiro[2.5]octan-7-yl]-2-[6-(methoxymethoxy)-2,7-dimethylindazol-5-yl]quinazoline-4-carboxylate (250 mg, 89%) as a solid. LCMS (ES, m/z): 617 [M+H]⁺.

### Synthesis of Intermediate C192

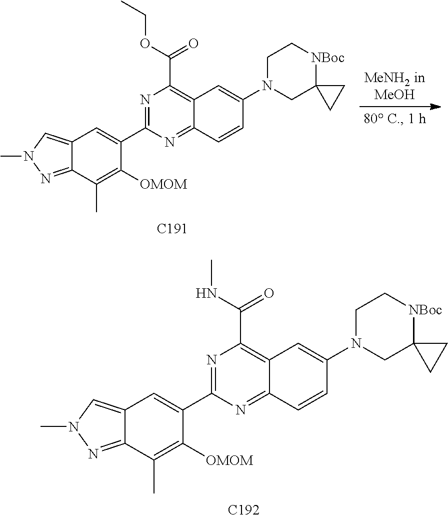

A mixture of ethyl 6-[4-(tert-butoxycarbonyl)-4,7-diazaspiro[2.5]octan-7-yl]-2-[6-(methoxymethoxy)-2,7-dimethylindazol-5-yl]quinazoline-4-carboxylate (250 mg, 0.405 mmol, 1 equiv) and 2M CH₃NH₂ in methanol (4 mL) was stirred for 1 h at 80° C. The resulting mixture was concentrated under reduced pressure to afford (230 mg) as a solid. LCMS (ES, m/z): 602 [M+H]⁺.

### Synthesis of Compound 532

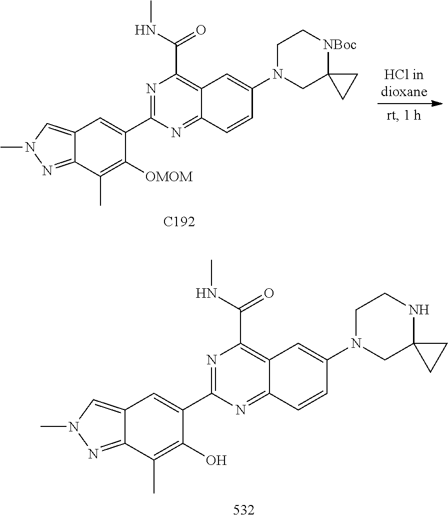

A solution of tert-butyl 7-{2-[6-(methoxymethoxy)-2,7-dimethylindazol-5-yl]-4-(methylcarbamoyl)quinazolin-6-yl}-4,7-diazaspiro[2.5]octane-4-carboxylate (200 mg, 0.332 mmol, 1 equiv) in DCM (4 mL) was treated with 4 M HCl (gas) in 1,4-dioxane (4 mL) for 1 h at room temperature. The resulting mixture was concentrated under reduced pressure to give a residue. The residue was purified by reverse flash chromatography (Condition 10, Gradient 9) to afford 6-{4,7-diazaspiro[2.5]octan-7-yl}-2-(6-hydroxy-2,7-dimethylindazol-5-yl)-N-methylquinazoline-4-carboxamide (70 mg, 46%) as a solid. LCMS (ES, m/z): 458 [M+H]⁺. ¹H NMR (300 MHz, DMSO-d₆) δ 13.40 (s, 1H), 9.21 (d, J=5.0 Hz, 1H), 9.10 (d, J=0.8 Hz, 1H), 8.42 (s, 1H), 8.12 (d, J=2.3 Hz, 1H), 8.06-7.92 (m, 2H), 4.17 (s, 3H), 3.39 (s, 2H), 3.28 (s, 2H), 3.10 (d, J=5.5 Hz, 2H), 2.97 (d, J=4.8 Hz, 3H), 2.41 (s, 3H), 0.71 (s, 4H).

### Example 177: Synthesis of Compound 536

### Synthesis of Intermediate C193

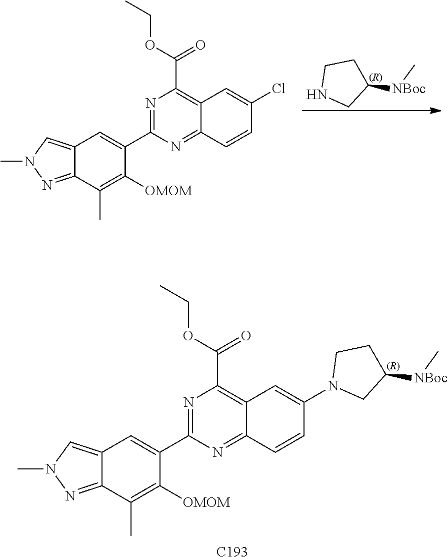

To a stirred mixture of ethyl 6-chloro-2-[6-(methoxymethoxy)-2,7-dimethylindazol-5-yl]quinazoline-4-carboxylate (200 mg, 0.454 mmol, 1 equiv) and tert-butyl N-methyl-N-[(3R)-pyrrolidin-3-yl]carbamate (136 mg, 0.681 mmol, 1.5 equiv) in 1,4-dioxane (4 mL) was added Cs₂CO₃ (443 mg, 1.362 mmol, 3 equiv), RuPhos (42 mg, 0.091 mmol, 0.2 equiv), and Pd₂(dba)₃ (41 mg, 0.045 mmol, 0.1 equiv) at room temperature under nitrogen atmosphere. The resulting mixture was stirred for 2 h at 100° C., then cooled to room temperature. The resulting mixture was concentrated under reduced pressure to give a residue. The residue was purified by silica gel column chromatography, eluted with PE/EA (1:1) to afford ethyl 6-[(3R)-3-[(tert-butoxycarbonyl)(methyl)amino]pyrrolidin-1-yl]-2-[6-(methoxymethoxy)-2,7-dimethylindazol-5-yl]quinazoline-4-carboxylate (200 mg, 73%) as a solid. LCMS (ES, m/z): 605 [M+H]⁺.

### Synthesis of Intermediate C194

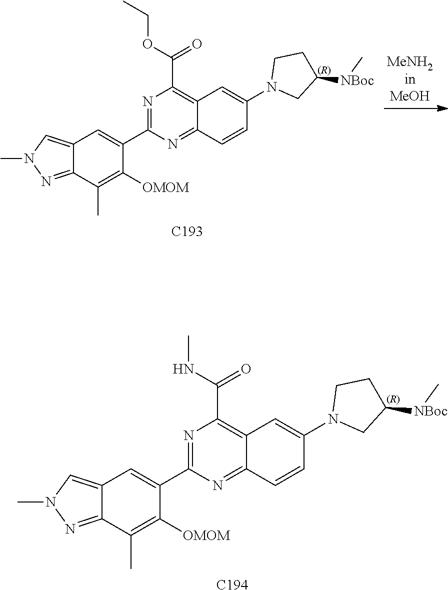

A mixture of ethyl 6-[(3R)-3-[(tert-butoxycarbonyl)(methyl)amino]pyrrolidin-1-yl]-2-[6-(methoxymethoxy)-2,7-dimethylindazol-5-yl]quinazoline-4-carboxylate (160 mg, 0.265 mmol, 1 equiv) and 2 M MeNH₂ solution in methanol (2 mL) was stirred for 3 h at 90° C., then cooled to room temperature. The resulting mixture was concentrated under vacuum to give a residue. The residue was purified by silica gel column chromatography, eluted with PE/EA (1:1) to afford tert-butyl N-[(3R)-1-{2-[6-(methoxymethoxy)-2,7-dimethylindazol-5-yl]-4-(methylcarbamoyl)quinazolin-6-yl}pyrrolidin-3-yl]-N-methylcarbamate (150 mg, 96%) as a solid. LCMS (ES, m/z): 590 [M+H]⁺.

### Synthesis of Compound 536

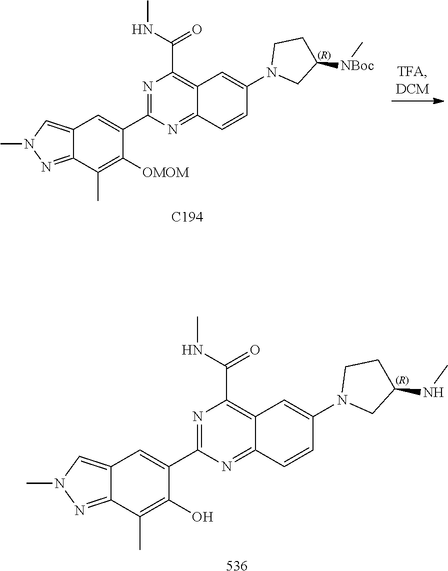

To a stirred solution of tert-butyl N-[(3R)-1-{2-[6-(methoxymethoxy)-2,7-dimethylindazol-5-yl]-4-(methylcarbamoyl)quinazolin-6-yl}pyrrolidin-3-yl]-N-methylcarbamate (150 mg, 0.254 mmol, 1 equiv) in DCM (1.5 mL) was added TFA (0.5 mL) at room temperature. The resulting mixture was stirred for 2 h at room temperature, then concentrated under reduced pressure to give a residue. The residue was purified by reversed-phase flash chromatography (Condition 10, Gradient 10) to afford 2-(6-hydroxy-2,7-dimethylindazol-5-yl)-N-methyl-6-[(3R)-3-(methylamino)pyrrolidin-1-yl]quinazoline-4-carboxamide (50 mg, 44%) as a solid. LCMS (ES, m/z): 446 [M+H]⁺. ¹H NMR (300 MHz, DMSO-d₆) δ 13.45 (s, 1H), 9.16 (d, J=5.1 Hz, 1H), 9.05 (s, 1H), 8.40 (s, 1H), 8.00 (d, J=9.3 Hz, 1H), 7.77 (d, J=2.7 Hz, 1H), 7.65-7.56 (m, 1H), 4.16 (s, 3H), 3.63 (s, 3H), 3.57 (d, J=8.1 Hz, 3H), 3.45 (d, J=7.7 Hz, 1H), 2.97 (d, J=4.7 Hz, 3H), 2.48 (s, 2H), 2.41 (s, 3H), 2.28 (s, 1H), 2.08 (s, 1H).

### Example 178: Synthesis of Compound 723

### Synthesis of Intermediate C195

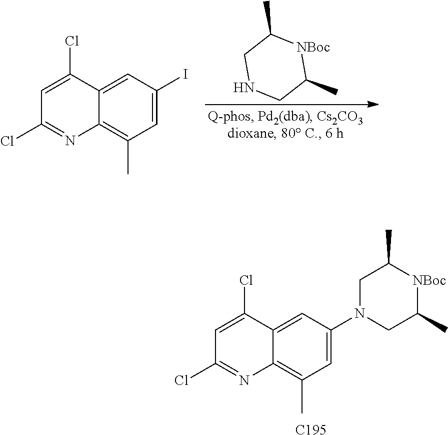 [SMILES: Cc1cc(I)cc2c(Cl)cc(Cl)nc12 ; C[C@@H]1CNC[C@H](C)N1C(=O)OC(C)(C)C ; Cc1cc(N2C[C@@H](C)N(C(=O)OC(C)(C)C)[C@@H](C)C2)cc2c(Cl)cc(Cl)nc12 ; "C195"]

To a stirred mixture of 2,4-dichloro-6-iodo-8-methylquinoline (300 mg, 0.888 mmol, 1.0 equiv) and tert-butyl (2R,6S)-2,6-dimethylpiperazine-1-carboxylate (190.2 mg, 0.888 mmol, 1.0 equiv) in dioxane (3 mL) was added Cs₂CO₃ (578.4 mg, 1.776 mmol, 2.0 equiv), Q-phos (62.9 mg, 0.089 mmol, 0.1 equiv), and Pd₂(dba)₃ (81.2 mg, 0.089 mmol, 0.1 equiv) at room temperature under nitrogen atmosphere. The resulting mixture was stirred for 6 h at 80° C. under nitrogen atmosphere, then cooled to room temperature, diluted with water (10 mL), and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with EA to afford tert-butyl (2R,6S)-4-(2,4-dichloro-8-methylquinolin-6-yl)-2,6-dimethylpiperazine-1-carboxylate (180 mg, 48%) as a solid. LCMS (ES, m/z): 424 [M+H]⁺.

### Synthesis of Intermediate C196

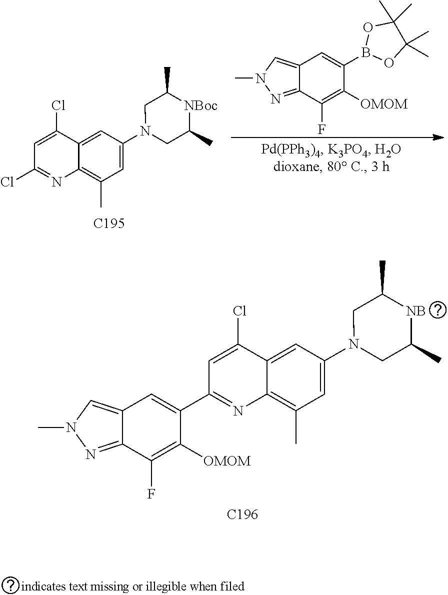

To a stirred mixture of tert-butyl (2R,6S)-4-(2,4-dichloro-8-methylquinolin-6-yl)-2,6-dimethylpiperazine-1-carboxylate (180 mg, 0.424 mmol, 1.0 equiv) and 7-fluoro-6-(methoxymethoxy)-2-methyl-5-(4,4,5,5-tetramethyl-1,3,2-dioxaborolan-2-yl) indazole (142.5 mg, 0.424 mmol, 1.0 equiv) in dioxane (1.5 mL) and water (0.3 mL) was added K₃PO₄ (180.0 mg, 0.848 mmol, 2.0 equiv) and Pd(PPh₃)₄(49.0 mg, 0.042 mmol, 0.1 equiv) at room temperature under nitrogen atmosphere. The resulting mixture was stirred for 3 h at 80° C. under nitrogen atmosphere, then cooled to room temperature, diluted with water (10 mL), and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by silica gel column chromatography, eluted with EA to afford tert-butyl (2R,6S)-4-{4-chloro-2-[7-fluoro-6-(methoxymethoxy)-2-methylindazol-5-yl]-8-methylquinolin-6-yl}-2,6-dimethylpiperazine-1-carboxylate (124 mg, 49%) as a solid. LCMS (ES, m/z): 598 [M+H]⁺.

### Synthesis of Compound 723

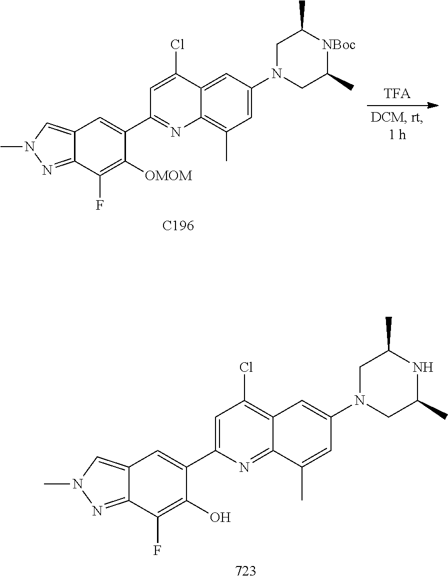

A solution of tert-butyl (2R,6S)-4-{4-chloro-2-[7-fluoro-6-(methoxymethoxy)-2-methylindazol-5-yl]-8-methylquinolin-6-yl}-2,6-dimethylpiperazine-1-carboxylate (120 mg, 0.201 mmol, 1 equiv) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 6, Gradient 6) to afford 5-{4-chloro-6-[(3R,5S)-3,5-dimethylpiperazin-1-yl]-8-methylquinolin-2-yl}-7-fluoro-2-methylindazol-6-ol (62 mg, 55%) as a solid. LCMS (ES, m/z): 454 [M+H]⁺. ¹H NMR (400 MHz, DMSO-d₆) δ 14.72 (s, 1H), 8.54 (d, J=8.5 Hz, 2H), 8.50 (d, J=2.6 Hz, 1H), 7.87-7.55 (m, 1H), 7.10 (d, J=2.6 Hz, 1H), 4.17 (s, 3H), 3.80 (dd, J=11.7, 2.8 Hz, 2H), 2.88-2.87 (m, 2H), 2.68 (s, 3H), 2.37-2.19 (m, 3H), 1.08 (d, J=6.2 Hz, 6H).

### Example 179: Synthesis of Compound 538

### Synthesis of Intermediate C197

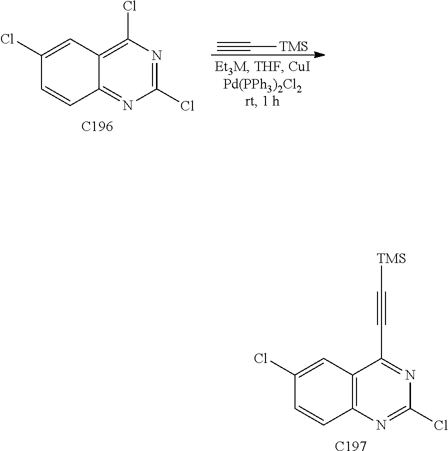

To a stirred mixture of 2,4,6-trichloroquinazoline (2.5 g, 10.708 mmol, 1.0 equiv) and trimethylsilylacetylene (1.05 g, 10.708 mmol, 1.0 equiv) in THE (50 mL) was added Et₃N (3.25 g, 32.124 mmol, 3.0 equiv) and bis(triphenylphosphane)dichloropalladium (0.75 g, 1.071 mmol, 0.1 equiv) at room temperature under nitrogen atmosphere. The resulting mixture was stirred for 2 h at room temperature under nitrogen atmosphere, then extracted with ethyl acetate (3×50 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with PE/EA (15:1) to afford 2,6-dichloro-4-[2-(trimethylsilyl)ethynyl]quinazoline (510 mg, 16%) as a solid. LCMS (ES, m/z): 395 [M+H]⁺.

### Synthesis of Intermediate C198

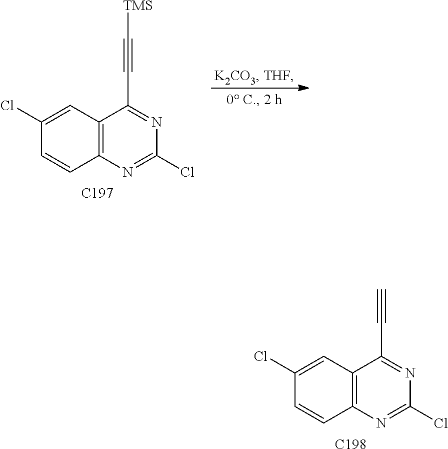 [SMILES: C[Si](C)(C)C#Cc1nc(Cl)nc2ccc(Cl)cc12 ; "C197" ; C#Cc1nc(Cl)nc2ccc(Cl)cc12 ; "C198"]

A solution of 2,6-dichloro-4-[2-(trimethylsilyl)ethynyl]quinazoline (470 mg, 1.592 mmol, 1.0 equiv) in THF (5 mL) was treated with K₂CO₃ (220.0 mg, 1.592 mmol, 1.0 equiv) at 0° C. The resulting mixture was stirred for 2 h at 0° C. under nitrogen atmosphere, then diluted with water (10 mL) and extracted with EtOAc (3×15 mL). The organic layers were combined, washed with water (1×20 mL) and brine (1×20 mL), dried over anhydrous Na₂SO₄, and filtered. The filtrate was concentrated under reduced pressure to give a residue. The residue was purified by silica gel column chromatography, eluted with PE/EA (4:1) to afford 2,6-dichloro-4-ethynylquinazoline (168 mg, 47%) as a solid. LCMS (ES, m/z): 223 [M+H]⁺.

### Synthesis of Intermediate C199

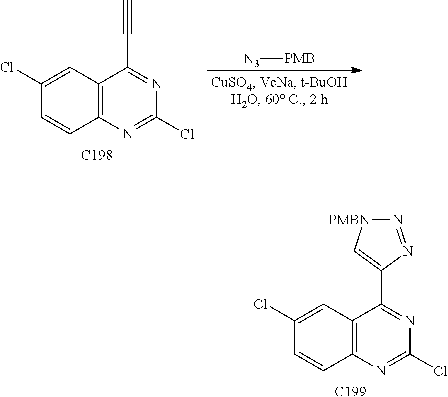

To a stirred mixture of 2,6-dichloro-4-ethynylquinazoline (162 mg, 0.628 mmol, 1.0 equiv) and 1-(azidomethyl)-4-methoxybenzene (204.8 mg, 1.256 mmol, 2.0 equiv) in tert-butanol (1.4 mL) and water (0.7 mL) was added CuSO₄ (10.02 mg, 0.063 mmol, 0.1 equiv) and sodium ascorbate (24.99 mg, 0.126 mmol, 0.2 equiv) at room temperature. The resulting mixture was stirred for 2 h at 60° C. under nitrogen atmosphere, then cooled to room temperature, diluted with water (40 mL), and extracted with ethyl acetate (3×30 mL). The organic layers were combined, washed with water (1×20 mL) and brine (1×20 mL), dried over anhydrous Na₂SO₄, and filtered. The filtrate was concentrated under reduced pressure to give a residue. The residue was purified by silica gel column chromatography, eluted with PE/EA (1:1) to afford 2,6-dichloro-4-{1-[(4-methoxyphenyl)methyl]-1,2,3-triazol-4-yl}quinazoline (140 mg, 58%) as a solid. LCMS (ES, m/z): 386 [M+H]⁺.

### Synthesis of Intermediate C200

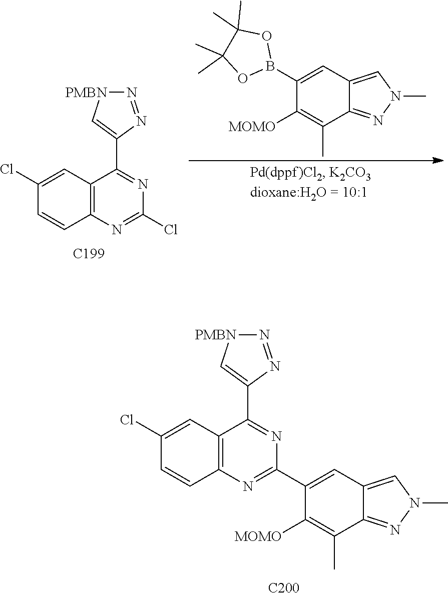

To a stirred mixture of 2,6-dichloro-4-{1-[(4-methoxyphenyl)methyl]-1,2,3-triazol-4-yl}quinazoline (123 mg, 0.318 mmol, 1.0 equiv) and 6-(methoxymethoxy)-2,7-dimethyl-5-(4,4,5,5-tetramethyl-1,3,2-dioxaborolan-2-yl) indazole (126.9 mg, 0.382 mmol, 1.2 equiv) in dioxane (1 mL) and water (0.2 mL) was added K₂CO₃ (88.0 mg, 0.636 mmol, 2 equiv) and Pd(dppf)Cl₂ (23.3 mg, 0.032 mmol, 0.1 equiv) at room temperature. The resulting mixture was stirred for 3 h at 80° C. under nitrogen atmosphere, then cooled to room temperature and concentrated under vacuum to give a reside. The residue was purified by silica gel column chromatography, eluted with PE/EA (1:2) to afford 6-chloro-2-[6-(methoxymethoxy)-2,7-dimethylindazol-5-yl]-4-{1-[(4-methoxyphenyl)methyl]-1,2,3-triazol-4-yl}quinazoline (136 mg, 77%) as a solid. LCMS (ES, m/z): 556 [M−H]⁺.

### Synthesis of Intermediate C201

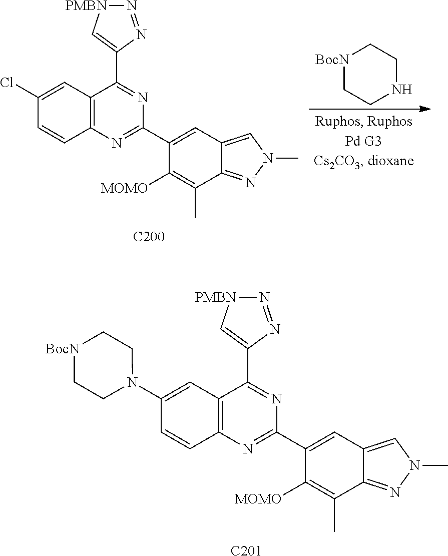

To a stirred mixture of 6-chloro-2-[6-(methoxymethoxy)-2,7-dimethylindazol-5-yl]-4-{1-[(4-methoxyphenyl)methyl]-1,2,3-triazol-4-yl}quinazoline (120 mg, 0.216 mmol, 1.0 equiv) and tert-butyl piperazine-1-carboxylate (48.2 mg, 0.259 mmol, 1.2 equiv) in dioxane (2.4 mL) was added Ruphos (20.1 mg, 0.043 mmol, 0.2 equiv), Cs₂CO₃ (140.6 mg, 0.432 mmol, 2 equiv), and RuPhos Palladacycle Gen.3 (18.0 mg, 0.022 mmol, 0.1 equiv) at room temperature under nitrogen atmosphere. The resulting mixture was stirred for 3 h at 110° C. under nitrogen atmosphere, then cooled to room temperature, diluted with water (40 mL), and extracted with ethyl acetate (3×50 mL). The organic layers were combined, washed with brine (1×30 mL), dried over anhydrous Na₂SO₄, and filtered. The filtrate was concentrated under reduced pressure to give a residue. The residue was purified by silica gel column chromatography, eluted with PE/EA (3:1) to afford tert-butyl 4-{2-[6-(methoxymethoxy)-2,7-dimethylindazol-5-yl]-4-{1-[(4-methoxyphenyl)methyl]-1,2,3-triazol-4-yl}quinazolin-6-yl}piperazine-1-carboxylate (152 mg, 88%) as a solid. LCMS (ES, m/z): 706 [M+H]⁺.

### Synthesis of Compound 538

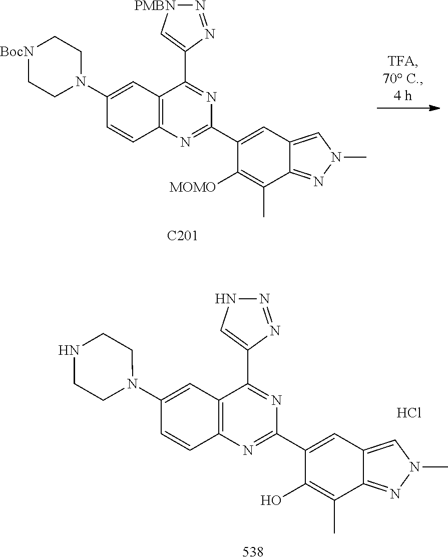

A mixture of tert-butyl 4-{2-[6-(methoxymethoxy)-2,7-dimethylindazol-5-yl]-4-{1-[(4-methoxyphenyl)methyl]-1,2,3-triazol-4-yl}quinazolin-6-yl}piperazine-1-carboxylate (150 mg, 0.213 mmol, 1 equiv) and TFA (2 mL) was stirred for 4 h at 70° C. under nitrogen atmosphere. The resulting mixture was concentrated under reduced pressure to give a residue. The residue was purified by Prep-HPLC (Condition 8, Gradient 2) to afford 2,7-dimethyl-5-[6-(piperazin-1-yl)-4-(1H-1,2,3-triazol-4-yl)quinazolin-2-yl]indazol-6-ol hydrochloride (32 mg, 23%) as a solid. LCMS (ES, m/z): 442 [M+H]⁺. ¹H NMR (300 MHz, D₂O+CD₃CN) δ 8.96 (s, 1H), 8.90 (s, 1H), 8.69 (s, 1H), 8.44 (s, 1H), 8.05 (s, 2H), 4.45 (s, 3H), 3.90 (d, J=5.9 Hz, 4H), 3.77 (d, J=5.7 Hz, 4H), 2.48 (s, 3H).

### Example 180: Synthesis of Compound 533

### Synthesis of Intermediate C202

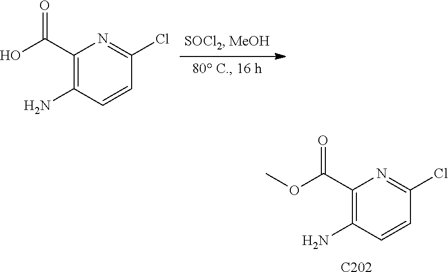

A solution of 3-amino-6-chloropyridine-2-carboxylic acid (10 g, 57.947 mmol, 1.0 equiv) in methanol (100 mL) was treated with SOCl₂ (6.89 g, 57.947 mmol, 1.0 equiv) at room temperature. The resulting mixture was stirred overnight at 80° C., then cooled to room temperature. The resulting mixture was concentrated under reduced pressure to give a residue. The residue was neutralized to pH 7 with saturated Na₂CO₃ (aq.) and extracted with CH₂Cl₂ (2×150 mL). The organic layers were combined, washed with water (1×100 mL) and brine (1×100 mL), dried over anhydrous Na₂SO₄, and filtered. The filtrate was concentrated under reduced pressure to afford methyl 3-amino-6-chloropyridine-2-carboxylate (9.7 g, 90%) as a solid. LCMS (ES, m/z): 187 [M+H]⁺.

### Synthesis of Intermediate C203

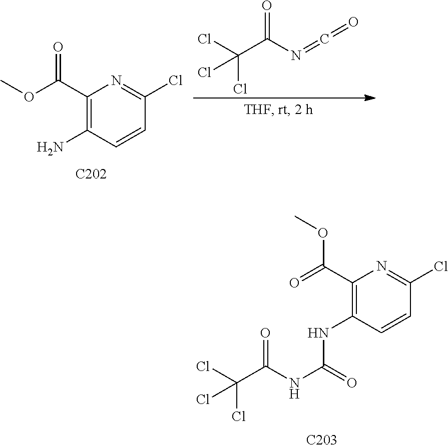

To a stirred solution of methyl 3-amino-6-chloropyridine-2-carboxylate (9.7 g, 51.983 mmol, 1.0 equiv) in THE (100 mL) was added trichloroethanecarbonyl isocyanate (9.79 g, 51.983 mmol, 1.0 equiv) dropwise at 0° C. under nitrogen atmosphere. The resulting mixture was stirred for 2 h at room temperature under nitrogen atmosphere, then diluted with diethyl ether (150 mL). A precipitate formed that was collected by filtration and washed with diethyl ether (1×20 mL). The resulting solid was dried to afford methyl 6-chloro-3-{[(2,2,2-trichloroacetyl)carbamoyl]amino}pyridine-2-carboxylate (19.2 g, 99%) as a solid. LCMS (ES, m/z): 374 [M+H]⁺.

### Synthesis of Intermediate C204

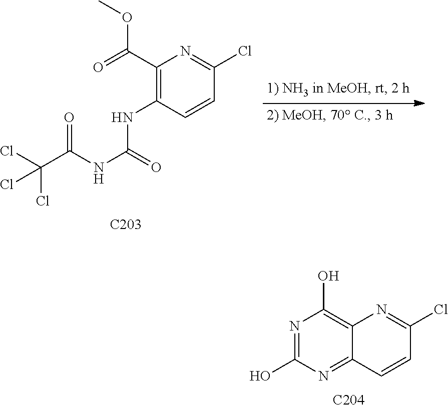 [SMILES: COC(=O)c1nc(Cl)ccc1NC(=O)NC(=O)C(Cl)(Cl)Cl ; "C203" ; Oc1nc(O)c2nc(Cl)ccc2n1 ; "C204"]

A mixture of methyl 6-chloro-3-{[(2,2,2-trichloroacetyl)carbamoyl]amino}pyridine-2-carboxylate (19.2 g, 51.203 mmol, 1.0 equiv) and NH₃(g) in methanol (1.45 mL, 51.203 mmol, 1 equiv) (7M) in methanol (250 mL) was stirred for 2 h at room temperature. The resulting mixture was heated to 70° C. and stirred for an additional 3 h. A precipitate formed that was collected by filtration and washed with methanol (1×50 mL). The resulting solid was dried to afford 6-chloropyrido[3,2-d]pyrimidine-2,4-diol (9.2 g, 91%) as a solid. LCMS (ES, m/z): 198 [M+H]⁺.

### Synthesis of Intermediate C205

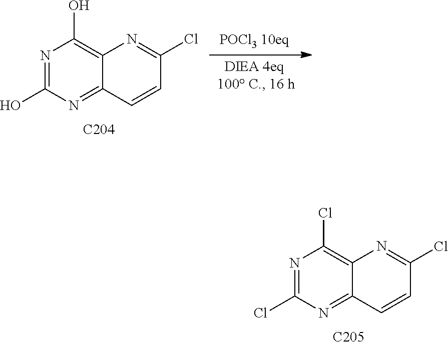 [SMILES: Oc1nc(O)c2nc(Cl)ccc2n1 ; "C204" ; Clc1ccc2nc(Cl)nc(Cl)c2n1 ; "C205"]

A solution of 6-chloropyrido[3,2-d]pyrimidine-2,4-diol (9 g, 45.551 mmol, 1.0 equiv) in POCl₃ (69.84 g, 455.510 mmol, 10.0 equiv) was treated with DIEA (23.55 g, 182.204 mmol, 4.0 equiv) at room temperature. The resulting mixture was stirred for 16 h at 100° C., then cooled to room temperature. The resulting mixture was concentrated under reduced pressure to give a residue. The residue was purified by silica gel column chromatography, eluted with PE/EA (5:1) to afford 2,4,6-trichloropyrido[3,2-d]pyrimidine (3.8 g, 36%) as a solid. LCMS (ES, m/z): 234 [M+H]⁺.

### Synthesis of Intermediate C206

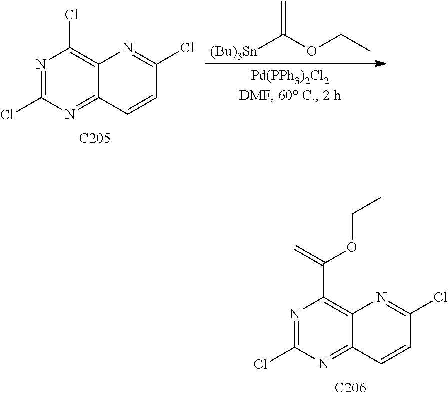

To a stirred mixture of 2,4,6-trichloropyrido[3,2-d]pyrimidine (3.6 g, 15.354 mmol, 1.0 equiv) and tributyl(1-ethoxyethenyl)stannane (5.55 g, 15.354 mmol, 1.0 equiv) in DMF (24 mL) was added palladium chloride; bis(triphenylphosphine) (1.08 g, 1.535 mmol, 0.1 equiv) at room temperature under nitrogen atmosphere. The resulting mixture was stirred for 2 h at 60° C. under nitrogen atmosphere. The mixture was allowed to cool down to room temperature. The resulting mixture was extracted with EtOAc (3×50 mL). The combined organic layers were washed with water (1×20 mL) and brine (1×20 mL), dried over anhydrous Na₂SO₄. After filtration, the filtrate was concentrated under reduced pressure. The residue was purified by silica gel column chromatography, eluted with PE/EA (5:1) to afford 2,6-dichloro-4-(1-ethoxyethenyl)pyrido[3,2-d]pyrimidine (2.4 g, 60%) as a light yellow solid. LCMS (ES, m/z): 270 [M+H]⁺.

### Synthesis of Intermediate C207

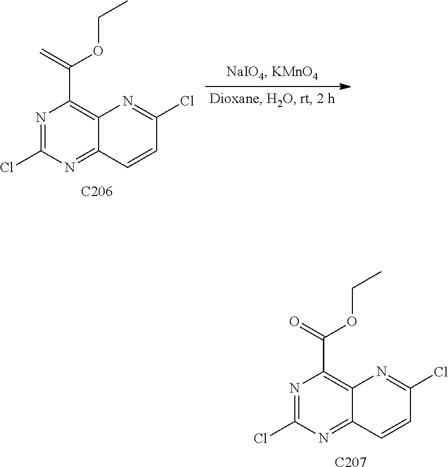 [SMILES: C=C(OCC)c1nc(Cl)nc2ccc(Cl)nc12 ; "C206" ; CCOC(=O)c1nc(Cl)nc2ccc(Cl)nc12 ; "C207"]

A solution of 2,6-dichloro-4-(1-ethoxyethenyl)pyrido[3,2-d]pyrimidine (2.2 g, 8.145 mmol, 1.0 equiv) in dioxane (22 mL) was treated with NaIO₄ (3.48 g, 16.290 mmol, 2.0 equiv) in water (10 mL) at room temperature under nitrogen atmosphere followed by the addition of KMnO₄ (643.5 mg, 4.072 mmol, 0.5 equiv) in portions at room temperature. The resulting mixture was stirred for 2 h at room temperature under nitrogen atmosphere, then extracted with ethyl acetate (3×50 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with PE/EA (4:1) to afford ethyl 2,6-dichloropyrido[3,2-d]pyrimidine-4-carboxylate (750 mg, 34%) as a solid. LCMS (ES, m/z): 272 [M+H]⁺.

### Synthesis of Intermediate C208

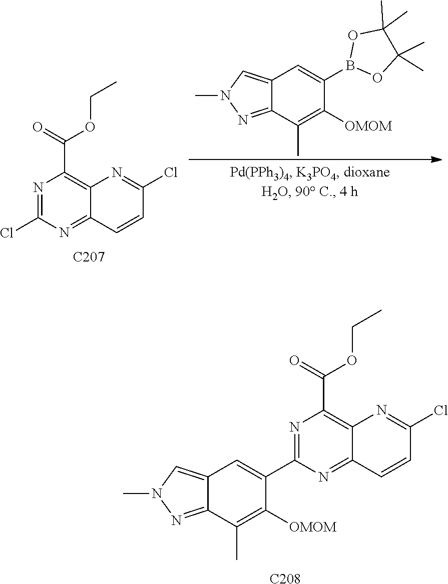

To a stirred mixture of ethyl 2,6-dichloropyrido[3,2-d]pyrimidine-4-carboxylate (750 mg, 2.756 mmol, 1.0 equiv) and 6-(methoxymethoxy)-2,7-dimethyl-5-(4,4,5,5-tetramethyl-1,3,2-dioxaborolan-2-yl) indazole (1.0 g, 3.032 mmol, 1.1 equiv) in dioxane (8 mL) and water (2 mL) was added K₃PO₄ (1.17 g, 5.512 mmol, 2.0 equiv) and Pd(PPh₃)₄(318.53 mg, 0.276 mmol, 0.1 equiv) at room temperature under nitrogen atmosphere. The resulting mixture was stirred for 4 h at 90° C. under nitrogen atmosphere, then cooled to room temperature and concentrated under vacuum to give a residue. The residue was purified by silica gel column chromatography, eluted with PE/EA (3:1) to afford ethyl 6-chloro-2-[6-(methoxymethoxy)-2,7-dimethylindazol-5-yl]pyrido[3,2-d]pyrimidine-4-carboxylate (260 mg, 21%) as a solid. LCMS (ES, m/z): 442 [M+H]⁺.

### Synthesis of Intermediate C209

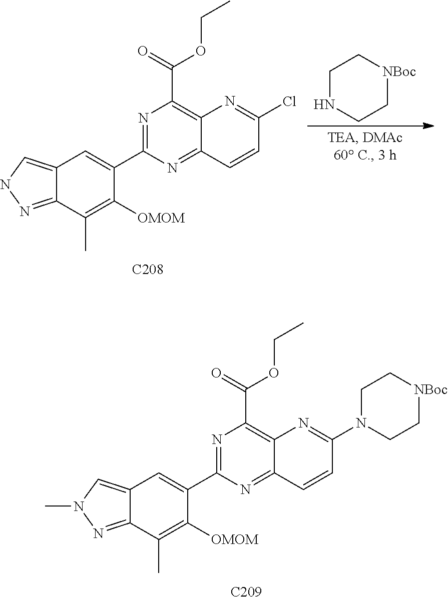

To a stirred mixture of ethyl 6-chloro-2-[6-(methoxymethoxy)-2,7-dimethylindazol-5-yl]pyrido[3,2-d]pyrimidine-4-carboxylate (130 mg, 0.294 mmol, 1.0 equiv) and tert-butyl piperazine-1-carboxylate (54.8 mg, 0.294 mmol, 1.0 equiv) in DMAc (2 mL) was added TEA (89.3 mg, 0.882 mmol, 3.0 equiv) at room temperature under nitrogen atmosphere. The resulting mixture was stirred for 3 h at 60° C. under nitrogen atmosphere, then cooled to room temperature and concentrated under reduced pressure to give a residue. LCMS (ES, m/z): 592 [M+H]⁺.

### Synthesis of Intermediate C210

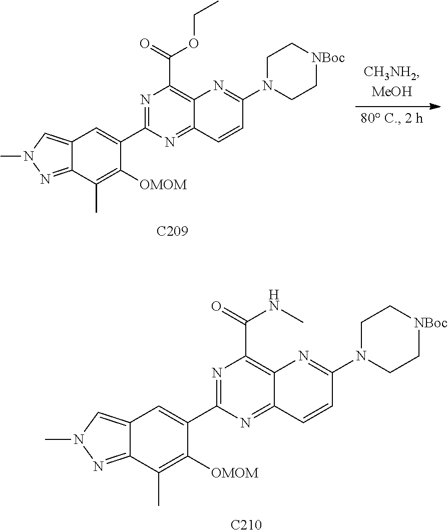

To C209 was added CH₃NH₂ (1.5 mL, 2M in MeOH) in methanol (10 mL) at room temperature. The resulting mixture was stirred for 2 h at 80° C., then cooled to room temperature and extracted with ethyl acetate (3×10 mL). The organic layers were combined, washed with brine (1×10 mL), dried over anhydrous Na₂SO₄, and filtered. The filtrate was concentrated under reduced pressure to give a residue. The residue was purified by Prep-TLC (EA) to afford tert-butyl 4-{2-[6-(methoxymethoxy)-2,7-dimethylindazol-5-yl]-4-(methylcarbamoyl)pyrido[3,2-d]pyrimidin-6-yl}piperazine-1-carboxylate (120 mg, 71%) as a solid. LCMS (ES, m/z): 577 [M+H]⁺.

### Synthesis of Compound 533

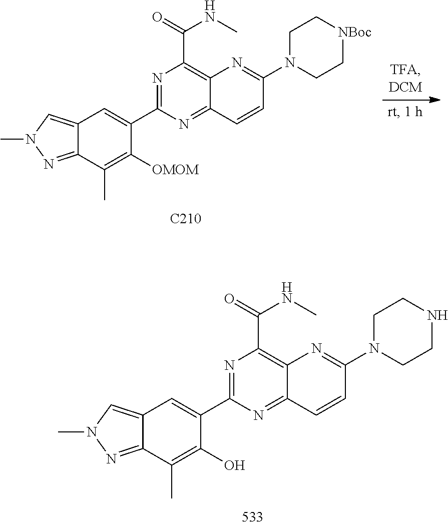

A solution of tert-butyl 4-{2-[6-(methoxymethoxy)-2,7-dimethylindazol-5-yl]-4-(methylcarbamoyl)pyrido[3,2-d]pyrimidin-6-yl}piperazine-1-carboxylate (114 mg, 0.198 mmol, 1.0 equiv) in DCM (1 mL) was treated with TFA (0.25 mL) at room temperature. The resulting mixture was stirred for 1 h at room temperature under nitrogen atmosphere. The resulting mixture was concentrated under reduced pressure to give a residue. The residue was purified by Prep-HPLC (Condition 6, Gradient 7) to afford 2-(6-hydroxy-2,7-dimethylindazol-5-yl)-N-methyl-6-(piperazin-1-yl)pyrido[3,2-d]pyrimidine-4-carboxamide (56.8 mg, 66%) as a solid. LCMS (ES, m/z): 433 [M+H]⁺. ¹H NMR (300 MHz, DMSO-d₆) δ 13.26 (s, 1H), 8.87 (d, J=5.0 Hz, 1H), 8.60 (d, J=9.5 Hz, 1H), 8.45 (s, 1H), 8.36 (s, 1H), 8.05 (d, J=9.2 Hz, 1H), 4.14 (s, 3H), 3.86 (d, J=5.7 Hz, 4H), 2.91 (d, J=4.7 Hz, 3H), 2.81 (t, J=5.2 Hz, 4H), 2.38 (s, 3H).

### Example 181: Synthesis of Compound 679

### Synthesis of Intermediate C211

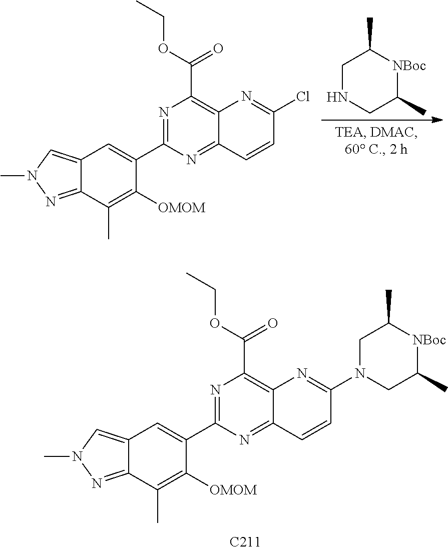

To a stirred mixture of ethyl 6-chloro-2-[6-(methoxymethoxy)-2,7-dimethylindazol-5-yl]pyrido[3,2-d]pyrimidine-4-carboxylate (120 mg, 0.272 mmol, 1.0 equiv) and tert-butyl (2R,6S)-2,6-dimethylpiperazine-1-carboxylate (67.6 mg, 0.316 mmol, 1.0 equiv) in DMAc (2 mL) was added TEA (95.8 mg, 0.948 mmol, 3.0 equiv) at room temperature under nitrogen atmosphere. The resulting mixture was stirred for 2 h at 60° C. under nitrogen atmosphere, then cooled to room temperature. The resulting mixture was concentrated under reduced pressure to give a residue. LCMS (ES, m/z): 620 [M+H]⁺.

### Synthesis of Intermediate C212

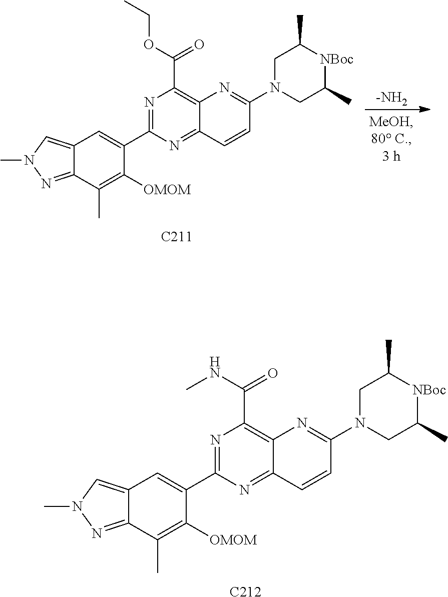

To C₂₁₁ was added CH₃NH₂ (1.5 mL, 2M in MeOH) in methanol (10 mL) at room temperature. The resulting mixture was stirred for an additional 3 h at 80° C., then cooled to room temperature and extracted with ethyl acetate (3×10 mL). The organic layers were combined, washed with water (1×10 mL) and brine (1×10 mL), dried over anhydrous Na₂SO₄, and filtered. The filtrate was concentrated under reduced pressure to give a residue. The residue was purified by Prep-TLC (EA) to afford tert-butyl (2R,6S)-4-{2-[6-(methoxymethoxy)-2,7-dimethylindazol-5-yl]-4-(methylcarbamoyl)pyrido[3,2-d]pyrimidin-6-yl}-2,6-dimethylpiperazine-1-carboxylate (121 mg) as a solid. LCMS (ES, m/z): 577 [M+H]⁺.

### Synthesis of Compound 679

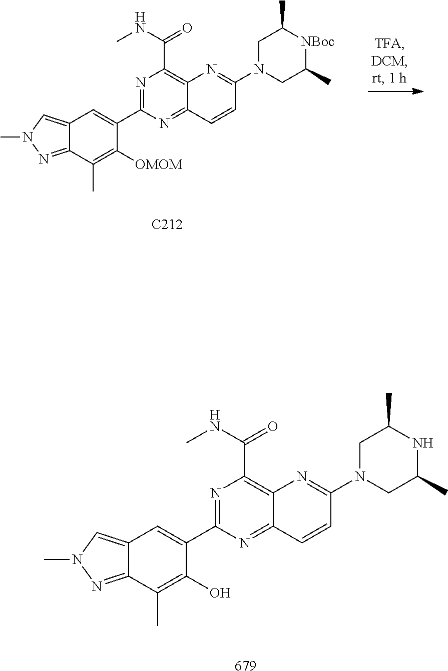

A solution of tert-butyl (2R,6S)-4-{2-[6-(methoxymethoxy)-2,7-dimethylindazol-5-yl]-4-(methylcarbamoyl)pyrido[3,2-d]pyrimidin-6-yl}-2,6-dimethylpiperazine-1-carboxylate (121 mg, 0.200 mmol, 1.0 equiv) in DCM (1 mL) was treated with TFA (0.25 mL) at room temperature. The resulting mixture was stirred for 1 h at room temperature under nitrogen atmosphere, then concentrated under reduced pressure to give a residue. The residue was purified by Prep-HPLC (Condition 6, Gradient 7) to afford 6-[(3R,5S)-3,5-dimethylpiperazin-1-yl]-2-(6-hydroxy-2,7-dimethylindazol-5-yl)-N-methylpyrido[3,2-d]pyrimidine-4-carboxamide (60.2 mg, 65%) as a solid. LCMS (ES, m/z): 461 [M+H]⁺. ¹H NMR (300 MHz, DMSO-d₆) δ 13.25 (s, 1H), 8.87 (d, J=4.9 Hz, 1H), 8.60 (d, J=9.5 Hz, 1H), 8.45 (s, 1H), 8.36 (s, 1H), 8.06 (d, J=9.3 Hz, 1H), 4.72 (d, J=12.3 Hz, 2H), 4.14 (s, 3H), 2.92 (d, J=4.7 Hz, 3H), 2.74 (d, J=8.3 Hz, 2H), 2.48 (d, J=2.8 Hz, 2H), 2.37 (s, 3H), 1.08 (d, J=6.2 Hz, 6H).

### Example 182: Synthesis of Compound 515

### Synthesis of Intermediate C213

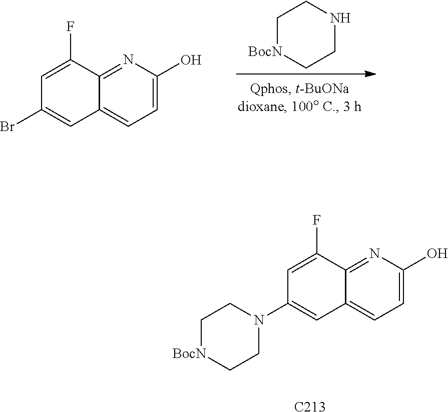 [SMILES: Oc1ccc2cc(Br)cc(F)c2n1 ; CC(C)(C)OC(=O)N1CCNCC1 ; CC(C)(C)OC(=O)N1CCN(c2cc(F)c3nc(O)ccc3c2)CC1 ; "C213"]

To a stirred mixture of 6-bromo-8-fluoroquinolin-2-ol (600 mg, 2.479 mmol, 1 equiv) and tert-butyl piperazine-1-carboxylate (692 mg, 3.719 mmol, 1.5 equiv) in 1,4-dioxane (5 mL) was added sodium t-butoxide (476 mg, 4.958 mmol, 2 equiv), Pd₂(dba)₃ (227 mg, 0.248 mmol, 0.1 equiv), and 1,2,3,4,5-pentaphenyl-1-(di-tert-butylphosphino)ferrocene (352 mg, 0.496 mmol, 0.2 equiv) at room temperature under nitrogen atmosphere. The resulting mixture was stirred for 3 h at 100° C., then cooled to room temperature. The reaction mixture was concentrated under reduced pressure to give a residue. The residue was purified by silica gel column chromatography, eluted with PE/EA (1:1) to afford tert-butyl 4-(8-fluoro-2-hydroxyquinolin-6-yl)piperazine-1-carboxylate (600 mg, 70%) as a solid. LCMS (ES, m/z): 348 [M+H]⁺.

### Synthesis of Intermediate C214

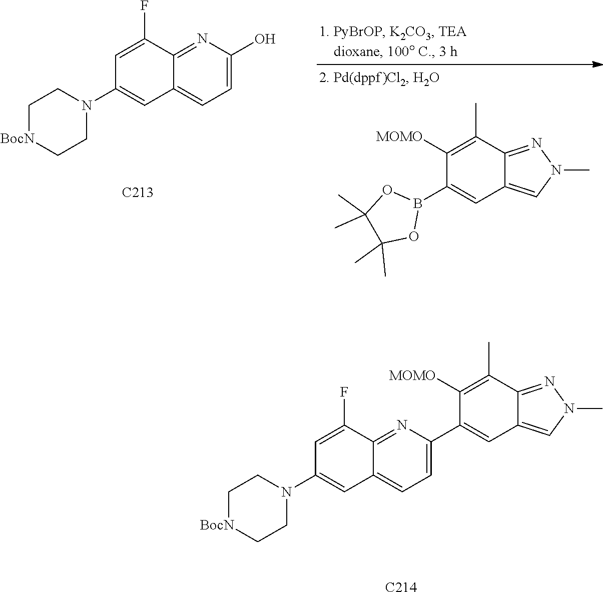

To a stirred mixture of tert-butyl 4-(8-fluoro-2-hydroxyquinolin-6-yl)piperazine-1-carboxylate (200 mg, 0.576 mmol, 1 equiv) and hexafluoro-l[5]-phosphanuide tris(pyrrolidin-1-yl)phosphanium bromide (402 mg, 0.864 mmol, 1.5 equiv) in 1,4-dioxane (5.0 mL) was added K₂CO₃ (238 mg, 1.728 mmol, 3 equiv) and TEA (174 mg, 1.728 mmol, 3 equiv) at room temperature under nitrogen atmosphere. The resulting mixture was stirred for 3 h at 100° C., then cooled to room temperature. To the reaction mixture was added 6-(methoxymethoxy)-2,7-dimethyl-5-(4,4,5-trimethyl-1,3,2-dioxaborolan-2-yl) indazole (219 mg, 0.691 mmol, 1.2 equiv), water (0.5 mL), and Pd(dppf)Cl₂ (46 mg, 0.058 mmol, 0.1 equiv) at room temperature under nitrogen atmosphere. The resulting mixture was stirred for an additional 2 h at 100° C., then cooled to room temperature. The resulting mixture was concentrated under reduced pressure to give a residue. The residue was purified by silica gel column chromatography, eluted with PE/EA (1:1) to afford tert-butyl 4-{8-fluoro-2-[6-(methoxymethoxy)-2,7-dimethylindazol-5-yl]quinolin-6-yl}piperazine-1-carboxylate (300 mg, 97%) as a solid. LCMS (ES, m/z): 536 [M+H]⁺.

### Synthesis of Compound 515

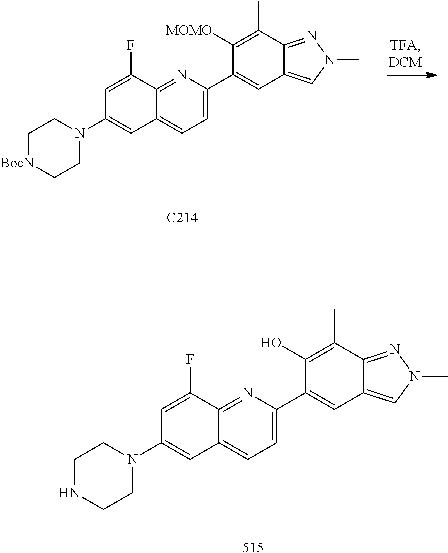

To a stirred solution of tert-butyl 4-{8-fluoro-2-[6-(methoxymethoxy)-2,7-dimethylindazol-5-yl]quinolin-6-yl}piperazine-1-carboxylate (200 mg, 0.373 mmol, 1 equiv) in DCM (1.5 mL) was added TFA (0.5 mL) at room temperature. The resulting mixture was stirred for 2 h at room temperature, then concentrated under reduced pressure to give a residue. The residue was purified by reverse flash chromatography (Condition 10, Gradient 10) to afford 5-[8-fluoro-6-(piperazin-1-yl)quinolin-2-yl]-2,7-dimethylindazol-6-ol (50 mg, 34%) as a solid. LCMS (ES, m/z): 392 [M+H]⁺. ¹H NMR (400 MHz, DMSO-d₆) δ 14.49 (s, 1H), 8.50 (s, 1H), 8.42-8.35 (m, 3H), 8.34 (d, J=8.6 Hz, 1H), 7.60 (dd, J=14.3, 2.4 Hz, 1H), 7.10 (d, J=2.5 Hz, 1H), 4.15 (s, 3H), 3.28 (d, J=10.0 Hz, 4H), 2.90 (t, J=5.1 Hz, 4H), 2.39 (s, 3H).

### Example 183: Synthesis of Compound 516

### Synthesis of Intermediate C215

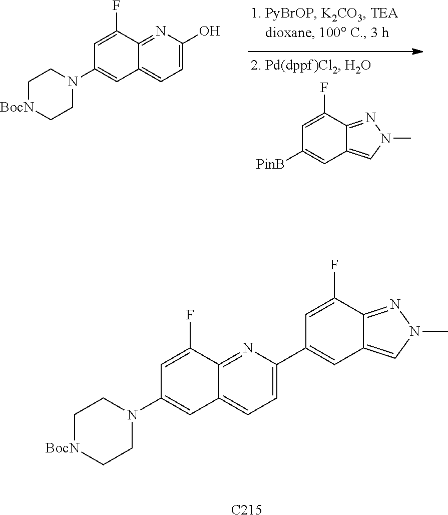

To a stirred mixture of tert-butyl 4-(8-fluoro-2-hydroxyquinolin-6-yl)piperazine-1-carboxylate (200 mg, 0.576 mmol, 1 equiv) and hexafluoro-l\hat{ }[5]-phosphanuide tris(pyrrolidin-1-yl)phosphanium bromide (402 mg, 0.864 mmol, 1.5 equiv) in 1,4-dioxane (2 mL, 0.023 mmol) was added K₂CO₃ (238 mg, 1.728 mmol, 3 equiv) and TEA (175 mg, 1.728 mmol, 3.0 equiv) at room temperature under nitrogen atmosphere. The resulting mixture was stirred for 3 h at 100° C., then cooled to room temperature. To the reaction in mixture was added 7-fluoro-2-methyl-5-(4,4,5,5-tetramethyl-1,3,2-dioxaborolan-2-yl) indazole (190 mg, 0.691 mmol, 1.2 equiv), water (0.4 mL), and Pd(dppf)Cl₂ (46 mg, 0.058 mmol, 0.1 equiv) at room temperature under nitrogen atmosphere. The resulting mixture was stirred for additional 2 h at 100° C., then cooled to room temperature. The resulting mixture was concentrated under reduced pressure to give a residue. The residue was purified by silica gel column chromatography, eluted with PE/EA (1:1) to afford tert-butyl 4-[8-fluoro-2-(7-fluoro-2-methylindazol-5-yl)quinolin-6-yl]piperazine-1-carboxylate (200 mg, 72%) as a solid. LCMS (ES, m/z): 480 [M+H]⁺.

### Synthesis of Compound 516

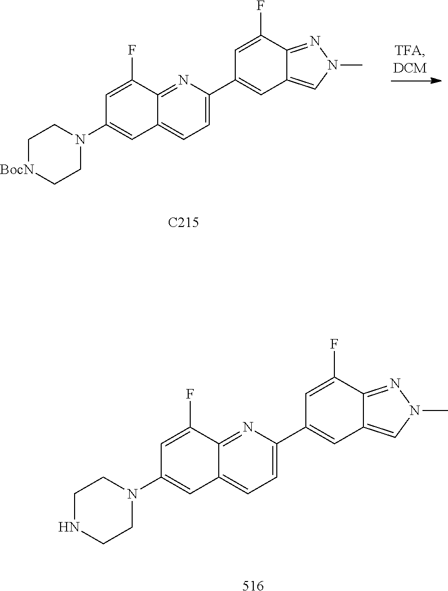 [SMILES: Cn1cc2cc(-c3ccc4cc(N5CCN(C(=O)OC(C)(C)C)CC5)cc(F)c4n3)cc(F)c2n1 ; "C215" ; Cn1cc2cc(-c3ccc4cc(N5CCNCC5)cc(F)c4n3)cc(F)c2n1 ; "516"]

To a stirred solution of tert-butyl 4-[8-fluoro-2-(7-fluoro-2-methylindazol-5-yl)quinolin-6-yl]piperazine-1-carboxylate (150 mg, 0.313 mmol, 1 equiv) in DCM (1.5 mL) was added TFA (0.5 mL) at room temperature. The resulting mixture was stirred for 2 h at room temperature, then concentrated under reduced pressure to give a residue. The residue was purified by reverse flash chromatography (Condition 10, Gradient 10) to afford 8-fluoro-2-(7-fluoro-2-methylindazol-5-yl)-6-(piperazin-1-yl)quinoline (25 mg, 21%) as a solid. LCMS (ES, m/z): 380 [M+H]⁺. ¹H NMR (300 MHz, DMSO-d₆) δ 8.63 (d, J=2.8 Hz, 1H), 8.44 (s, 1H), 8.25 (d, J=8.2 Hz, 1H), 8.18 (d, J=8.8 Hz, 1H), 7.99 (dd, J=13.7, 1.3 Hz, 1H), 7.52 (dd, J=14.3, 2.5 Hz, 1H), 7.05 (d, J=2.5 Hz, 1H), 4.24 (s, 3H), 3.27 (t, J=5.0 Hz, 4H), 2.92 (t, J=5.0 Hz, 4H).

### Example 184: Synthesis of Compound 517

### Synthesis of Intermediate C216

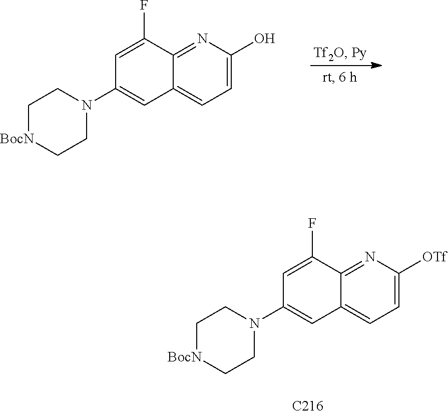 [SMILES: CC(C)(C)OC(=O)N1CCN(c2cc(F)c3nc(O)ccc3c2)CC1 ; CC(C)(C)OC(=O)N1CCN(c2cc(F)c3nc(OS(=O)(=O)C(F)(F)F)ccc3c2)CC1 ; "C216"]

A mixture of tert-butyl 4-(8-fluoro-2-hydroxyquinolin-6-yl) piperazine-1-carboxylate (200 mg, 0.576 mmol, 1.00 equiv) and Tf₂O (32 mg, 1.152 mmol, 2.00 equiv) in pyridine (3 mL) was stirred for 6 h at room temperature. The reaction mixture was quenched with water (10 mL) and extracted with ethyl acetate (3×10 mL). The organic layers were combined, washed with brine (3×10 mL), dried over anhydrous Na₂SO₄, and filtered. The filtrate was concentrated under reduced pressure to give a residue. The residue was purified by silica gel column chromatography, eluted with PE/EA (10:1) to afford tert-butyl 4-[8-fluoro-2-(trifluoromethanesulfonyloxy)quinolin-6-yl]piperazine-1-carboxylate (130 mg, 47%) as a solid. LCMS (ES, m/z): 479 [M+H]⁺.

### Synthesis of Intermediate C217

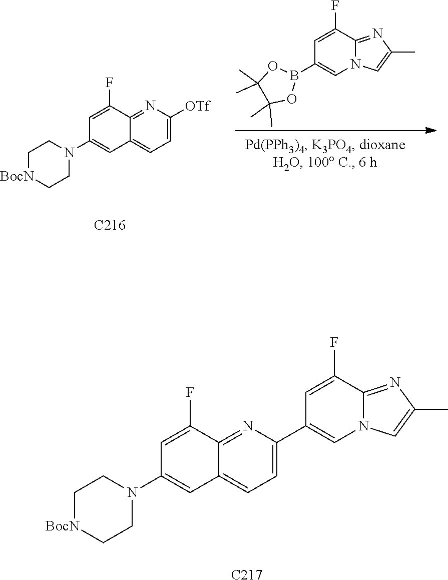

To a stirred mixture of tert-butyl 4-[8-fluoro-2-(trifluoromethanesulfonyloxy)quinolin-6-yl]piperazine-1-carboxylate (130 mg, 0.271 mmol, 1 equiv) and 8-fluoro-2-methyl-6-(4,4,5,5-tetramethyl-1,3,2-dioxaborolan-2-yl)imidazo[1,2-a]pyridine (112 mg, 0.407 mmol, 1.5 equiv) in dioxane (2 mL) and water (0.4 mL) was added K₃PO₄ (115 mg, 0.542 mmol, 2.0 equiv) and Pd(PPh₃)₄(31 mg, 0.027 mmol, 0.1 equiv) at room temperature under nitrogen atmosphere. The resulting mixture was stirred for 6 h at 100° C. under nitrogen atmosphere, then concentrated under reduced pressure to give a residue. The residue was purified by silica gel column chromatography, eluted with EA to afford tert-butyl 4-(8-fluoro-2-{8-fluoro-2-methylimidazo[1,2-a]pyridin-6-yl}quinolin-6-yl)piperazine-1-carboxylate (110 mg, 85%) as a solid. LCMS (ES, m/z): 480 [M+H]⁺.

### Synthesis of Compound 517

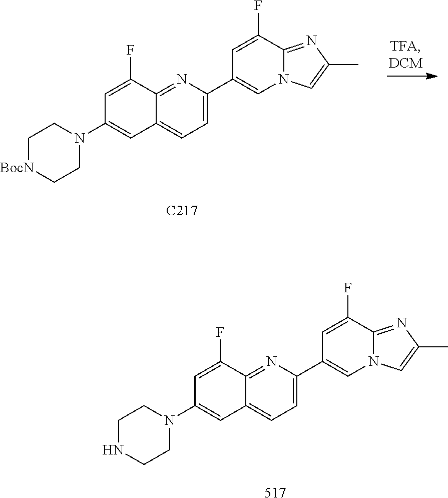 [SMILES: Cc1cn2cc(-c3ccc4cc(N5CCN(C(=O)OC(C)(C)C)CC5)cc(F)c4n3)cc(F)c2n1 ; "C217" ; Cc1cn2cc(-c3ccc4cc(N5CCNCC5)cc(F)c4n3)cc(F)c2n1 ; "517"]

A mixture of tert-butyl 4-(8-fluoro-2-{8-fluoro-2-methylimidazo[1,2-a]pyridin-6-yl}quinolin-6-yl)piperazine-1-carboxylate (100 mg, 0.209 mmol, 1 equiv) and trifluoroacetic acid (0.5 mL) in DCM (2 mL) was stirred for 2 h at room temperature. The resulting mixture was concentrated under reduced pressure to give a residue. The residue was purified by reverse flash chromatography (Condition 9, Gradient 4) to afford 8-fluoro-2-{8-fluoro-2-methylimidazo[1,2-a]pyridin-6-yl}-6-(piperazin-1-yl)quinoline (40 mg, 51%) as a solid. LCMS (ES, m/z): 380 [M+H]⁺. ¹H NMR (300 MHz, DMSO-d₆) δ 9.25 (s, 1H), 8.28 (d, J=8.9 Hz, 1H), 8.08 (d, J=8.8 Hz, 1H), 7.92 (d, J=12.9 Hz, 2H), 7.54 (d, J=13.9 Hz, 1H), 7.04 (s, 1H), 3.24 (d, J=6.0 Hz, 4H), 2.88 (s, 4H), 2.39 (s, 3H).

### Example 185: Synthesis of Compound 513

### Synthesis of Intermediate C218

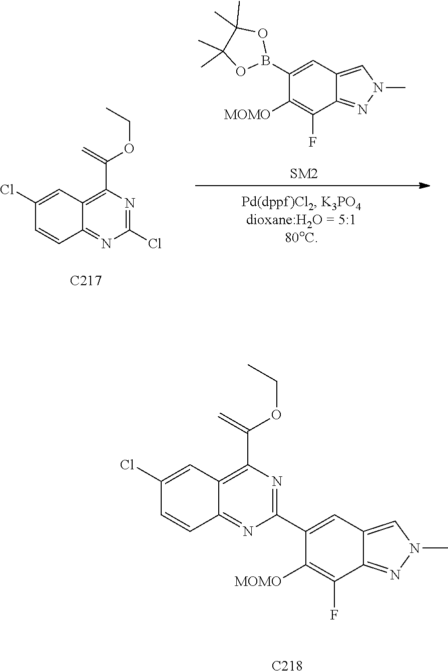

A mixture of 2,6-dichloro-4-(1-ethoxyethenyl)quinazoline (400 mg, 1.486 mmol, 1.0 equiv), 7-fluoro-6-(methoxymethoxy)-2-methyl-5-(4,4,5,5-tetramethyl-1,3,2-dioxaborolan-2-yl) indazole (499.64 mg, 1.486 mmol, 1.0 equiv), K₃PO₄ (473.22 mg, 2.229 mmol, 1.50 equiv), dioxane (1 mL), water (0.2 mL) and Pd(dppf)Cl₂ (121.07 mg, 0.149 mmol, 0.1 equiv) was stirred overnight at 80° C. under nitrogen atmosphere. The resulting mixture was concentrated under reduced pressure to give a residue. The residue was purified by silica gel column chromatography, eluted with PE/EA (1:3) to afford 6-chloro-4-(1-ethoxyethenyl)-2-[7-fluoro-6-(methoxymethoxy)-2-methylindazol-5-yl]quinazoline (400 mg, 61%) as a solid. LCMS (ES, m/z): 443 [M+H]⁺.

### Synthesis of Intermediate C219

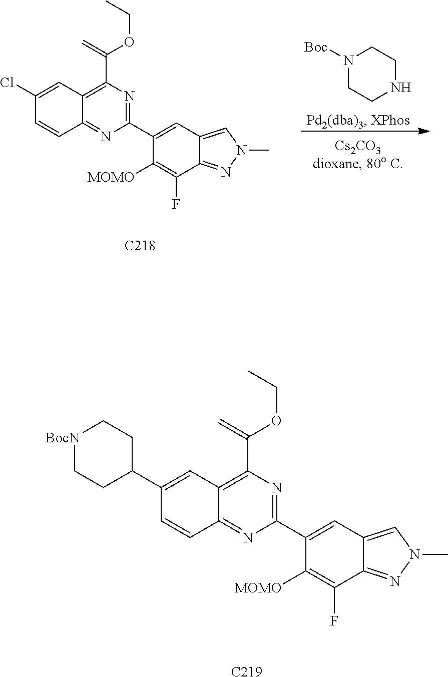

A mixture of 6-chloro-4-(1-ethoxyethenyl)-2-[7-fluoro-6-(methoxymethoxy)-2-methylindazol-5-yl]quinazoline (200 mg, 0.452 mmol, 1.0 equiv), tert-butyl piperazine-1-carboxylate (126.17 mg, 0.678 mmol, 1.5 equiv), X-Phos (43.06 mg, 0.090 mmol, 0.2 equiv), dioxane (4 mL), Pd₂(dba)₃ (41.35 mg, 0.045 mmol, 0.1 equiv) and Cs₂CO₃ (220.70 mg, 0.678 mmol, 1.5 equiv) was stirred overnight at 80° C. under nitrogen atmosphere. The resulting mixture was concentrated under reduced pressure to give a residue. The residue was purified by silica gel column chromatography, eluted with CH₂Cl₂/MeOH (20:1) to afford tert-butyl 4-[4-(1-ethoxyethenyl)-2-[7-fluoro-6-(methoxymethoxy)-2-methylindazol-5-yl]quinazolin-6-yl]piperazine-1-carboxylate (210 mg, 78%) as a solid. LCMS (ES, m/z): 593 [M+H]⁺.

### Synthesis of Compound 513

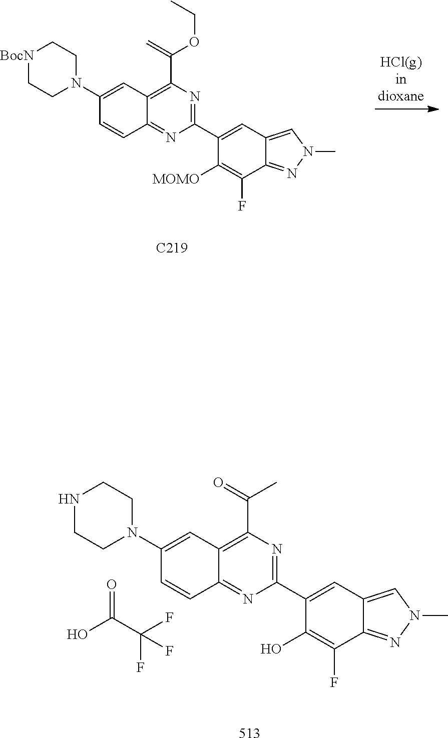

A mixture of tert-butyl 4-[4-(1-ethoxyethenyl)-2-[7-fluoro-6-(methoxymethoxy)-2-methylindazol-5-yl]quinazolin-6-yl]piperazine-1-carboxylate (200 mg, 0.337 mmol, 1 equiv) and HCl (gas) in 1,4-dioxane (5 mL) was stirred for 4 h at room temperature. The resulting mixture was concentrated under reduced pressure to give a residue. The residue was purified by reverse flash chromatography (Condition 4, Gradient 1) to afford 1-(2-(7-fluoro-6-hydroxy-2-methyl-2H-indazol-5-yl)-6-(piperazin-1-yl)quinazolin-4-yl)ethan-1-one 2,2,2-trifluoroacetate (35 mg, 25%) as a solid. LCMS (ES, m/z): 421 [M+H]⁺. ¹H NMR (400 MHz, DMSO-d₆) δ 13.21 (s, 1H), 8.91 (s, 1H), 8.83 (s, 2H), 8.59 (d, J=2.6 Hz, 1H), 8.15 (d, J=9.4 Hz, 1H), 8.06 (dd, J=9.5, 2.8 Hz, 1H), 7.98 (d, J=2.7 Hz, 1H), 4.19 (s, 3H), 3.59 (d, J=5.5 Hz, 4H), 3.34 (d, J=5.5 Hz, 4H), 2.93 (s, 3H).

### Example 186: Synthesis of Compound 663

### Synthesis of Intermediate C220

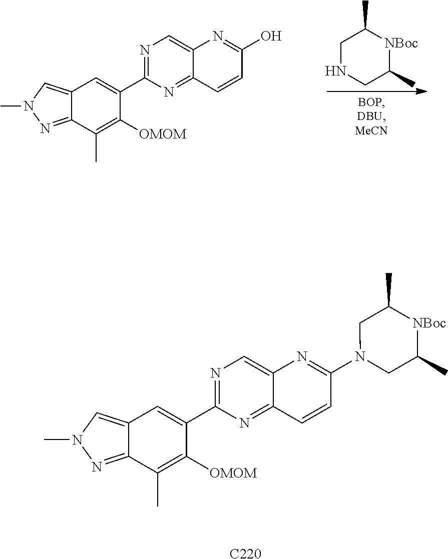

To a stirred mixture of 2-[6-(methoxymethoxy)-2,7-dimethylindazol-5-yl]pyrido[3,2-d]pyrimidin-6-ol (350 mg, 0.996 mmol, 1 equiv) and tert-butyl (2R,6S)-2,6-dimethylpiperazine-1-carboxylate (320 mg, 1.494 mmol, 1.5 equiv) in acetonitrile (10 mL) was added BOP (881 mg, 1.992 mmol, 2 equiv) and DBU (455 mg, 2.988 mmol, 3 equiv) at room temperature under nitrogen atmosphere. The resulting mixture was stirred for 24 h at 55° C. under nitrogen atmosphere, then cooled to room temperature, quenched with water (50 mL), and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by silica gel column chromatography, eluted with EA to afford tert-butyl (2R,6S)-4-[2-(6-hydroxy-2,7-dimethylindazol-5-yl)pyrido[3,2-d]pyrimidin-6-yl]-2,6-dimethylpiperazine-1-carboxylate (400 mg, 80%) as a solid. LCMS (ES, m/z): 548 [M+H]⁺.

### Synthesis of Compound 663

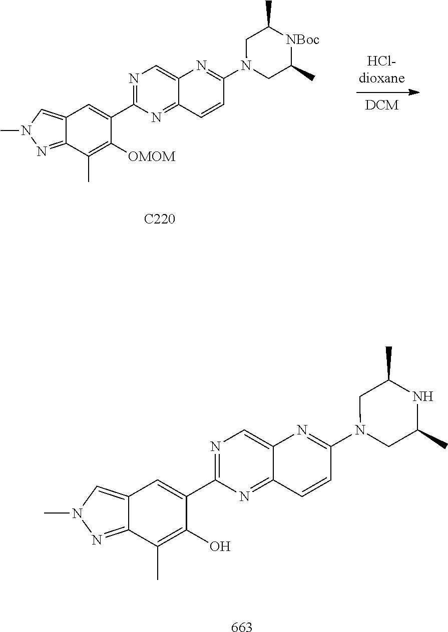

A mixture of tert-butyl (2R,6S)-4-{2-[6-(methoxymethoxy)-2,7-dimethylindazol-5-yl]pyrido[3,2-d]pyrimidin-6-yl}-2,6-dimethylpiperazine-1-carboxylate (168 mg, 0.307 mmol, 1 equiv) and 4 M HCl (gas) in 1,4-dioxane (1 mL) in DCM (3 mL) was stirred for 1 h at room temperature. The resulting mixture was concentrated under vacuum to give a residue. The residue was purified by reverse flash chromatography (Condition 10, Gradient 1) to afford 5-{6-[(3R,5S)-3,5-dimethylpiperazin-1-yl]pyrido[3,2-d]pyrimidin-2-yl}-2,7-dimethylindazol-6-ol (75 mg, 61%) as a solid. LCMS (ES, m/z): 404 [M+H]⁺. ¹H NMR (300 MHz, DMSO-d₆) δ 13.29 (s, 1H), 9.25 (s, 1H), 8.85 (s, 1H), 8.40 (s, 1H), 8.18 (d, J=9.5 Hz, 1H), 7.81 (d, J=9.6 Hz, 1H), 4.50 (d, J=12.4 Hz, 2H), 4.14 (s, 3H), 2.77 (s, 2H), 2.43 (d, J=23.9 Hz, 5H), 1.08 (d, J=6.1 Hz, 6H).

### Example 187: Synthesis of Compound 544

### Synthesis of Intermediate C221

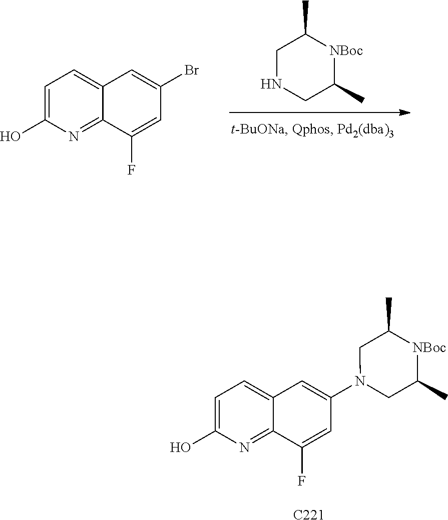 [SMILES: Oc1ccc2cc(Br)cc(F)c2n1 ; C[C@@H]1CNC[C@H](C)N1C(=O)OC(C)(C)C ; C[C@@H]1CN(c2cc(F)c3nc(O)ccc3c2)C[C@H](C)N1C(=O)OC(C)(C)C ; "C221"]

To a stirred mixture of 6-bromo-8-fluoroquinolin-2-ol (500 mg, 2.066 mmol, 1 equiv), t-BuONa (595 mg, 6.198 mmol, 3 equiv), and tert-butyl (2R,6S)-2,6-dimethylpiperazine-1-carboxylate (664 mg, 3.099 mmol, 1.5 equiv) in dioxane (10 mL) was added 1,2,3,4,5-pentaphenyl-1-(di-tert-butylphosphino)ferrocene (293 mg, 0.413 mmol, 0.2 equiv) and Pd₂(dba)₃ (189 mg, 0.207 mmol, 0.1 equiv) at room temperature under nitrogen atmosphere. The resulting mixture was stirred for 2 h at 100° C. under nitrogen atmosphere. The resulting mixture was concentrated under vacuum to give a residue. The residue was purified by silica gel column chromatography, eluted with PE/EA (1:8) to afford tert-butyl (2R,6S)-4-(8-fluoro-2-hydroxyquinolin-6-yl)-2,6-dimethylpiperazine-1-carboxylate (450 mg, 58%) as a solid. LCMS (ES, m/z): 376 [M+H]⁺.

### Synthesis of Intermediate C222

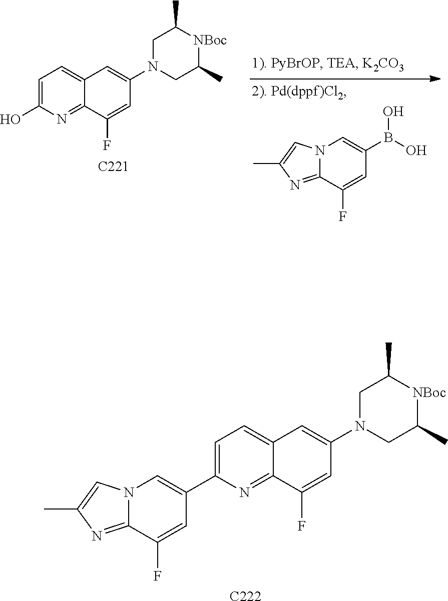

To a stirred mixture of tert-butyl (2R,6S)-4-(8-fluoro-2-hydroxyquinolin-6-yl)-2,6-dimethylpiperazine-1-carboxylate (460 mg, 1.225 mmol, 1 equiv) and PyBrOP (856 mg, 1.838 mmol, 1.5 equiv) in dioxane (5 mL) was added TEA (371 mg, 3.675 mmol, 3 equiv) and K₂CO₃ (508 mg, 3.675 mmol, 3 equiv) at room temperature. The resulting mixture was stirred for 3 h at 100° C., then cooled to room temperature. To the reaction mixture were added water (0.3 mL), (8-fluoro-2-methylimidazo[1,2-a]pyridin-6-yl)boronic acid (360 mg, 1.838 mmol, 1.5 equiv), and Pd(dppf)Cl₂ (89 mg, 0.123 mmol, 0.1 equiv) at room temperature. The resulting mixture was stirred for 16 h at 100° C. under nitrogen atmosphere, then cooled to room temperature. The resulting mixture was filtered and the filter cake washed with ethyl acetate (3×40 mL). The filtrate was concentrated under reduced pressure to give a residue. The residue was purified by silica gel column chromatography, eluted with EA/MeOH (10:1) to afford tert-butyl (2R,6S)-4-(8-fluoro-2-{8-fluoro-2-methylimidazo[1,2-a]pyridin-6-yl}quinolin-6-yl)-2,6-dimethylpiperazine-1-carboxylate (300 mg, 48%) as a solid. LCMS (ES, m/z): 508 [M+H]⁺.

### Synthesis of Compound 544

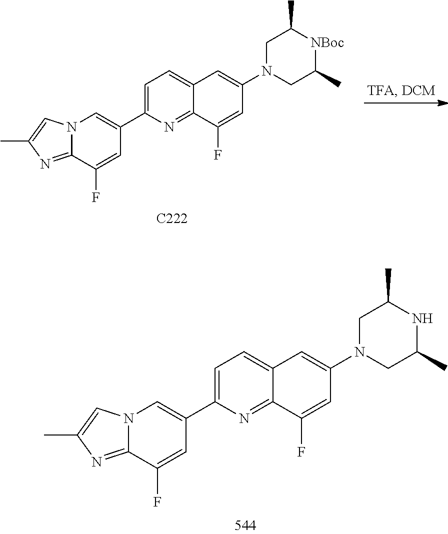

A mixture of tert-butyl (2R,6S)-4-(8-fluoro-2-{8-fluoro-2-methylimidazo[1,2-a]pyridin-6-yl}quinolin-6-yl)-2,6-dimethylpiperazine-1-carboxylate (250 mg, 0.493 mmol, 1 equiv) and trifluoroacetic acid (1 mL) in DCM (5 mL) was stirred for 2 h at room temperature. The resulting mixture was concentrated under reduced pressure to give a residue. The residue was purified by reversed-phase flash chromatography (Condition 10, Gradient 10) to afford 6-[(3R,5S)-3,5-dimethylpiperazin-1-yl]-8-fluoro-2-{8-fluoro-2-methylimidazo[1,2-a]pyridin-6-yl}quinoline (105 mg, 52%) as a solid. LCMS (ES, m/z): 408 [M+H]⁺. ¹H NMR (300 MHz, DMSO-d₆) δ 9.25 (d, J=1.4 Hz, 1H), 8.28 (dd, J=8.9, 1.7 Hz, 1H), 8.07 (d, J=8.8 Hz, 1H), 7.97-7.86 (m, 2H), 7.56 (dd, J=14.6, 2.5 Hz, 1H), 7.07-7.00 (m, 1H), 3.77 (d, J=11.4 Hz, 2H), 2.87 (s, 2H), 2.43-2.36 (m, 3H), 2.27 (t, J=11.1 Hz, 3H), 1.07 (d, J=6.2 Hz, 6H).

### Example 188: Synthesis of Compound 331

### Synthesis of Intermediate C223

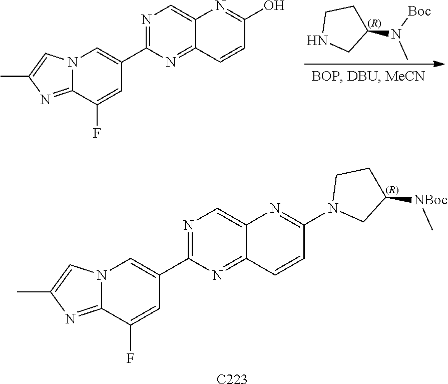

To a stirred mixture of 2-{8-fluoro-2-methylimidazo[1,2-a]pyridin-6-yl}pyrido[3,2-d]pyrimidin-6-ol (240 mg, 0.813 mmol, 1 equiv) and tert-butyl N-methyl-N-[(3R)-pyrrolidin-3-yl]carbamate (244 mg, 1.220 mmol, 1.5 equiv) in acetonitrile (10 mL) was added BOP (719 mg, 1.626 mmol, 2 equiv) and DBU (248 mg, 1.626 mmol, 2 equiv) at room temperature. The resulting mixture was stirred for overnight at room temperature, then quenched with water (50 mL) and extracted with ethyl acetate (3×20 mL). The organic layers were combined, washed with brine (1×20 mL), dried over anhydrous Na₂SO₄, and filtered. The filtrate was concentrated under reduced pressure to give a residue. The residue was purified by reverse flash chromatography (Condition 7, Gradient 1) to afford tert-butyl N-[(3R)-1-(2-{8-fluoro-2-methylimidazo[1,2-a]pyridin-6-yl}pyrido[3,2-d]pyrimidin-6-yl)pyrrolidin-3-yl]-N-methylcarbamate (220 mg, 57%) as a solid. LCMS (ES, m/z): 478 [M+H]⁺.

### Synthesis of Compound 331

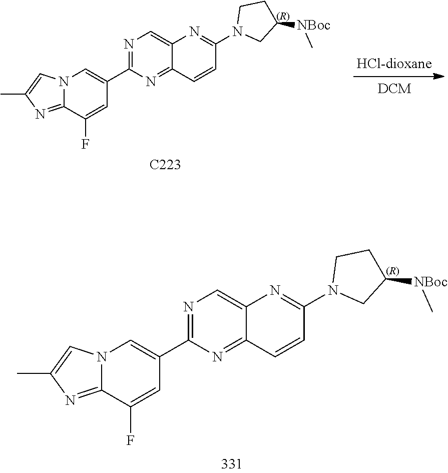

A mixture of tert-butyl N-[(3R)-1-(2-{8-fluoro-2-methylimidazo[1,2-a]pyridin-6-yl}pyrido[3,2-d]pyrimidin-6-yl)pyrrolidin-3-yl]-N-methylcarbamate (220 mg, 0.461 mmol, 1 equiv) and 4 M HCl (gas) in 1,4-dioxane (1 mL) in DCM (3 mL) was stirred for 1 h at room temperature under nitrogen atmosphere. The resulting mixture was concentrated under reduced pressure to give a residue. The residue was purified by reverse flash chromatography (Condition 10, Gradient 11) to afford (3R)-1-(2-{8-fluoro-2-methylimidazo[1,2-a]pyridin-6-yl}pyrido[3,2-d]pyrimidin-6-yl)-N-methylpyrrolidin-3-amine (120 mg, 69%) as a solid. LCMS (ES, m/z): 378 [M+H]⁺. ¹H NMR (300 MHz, DMSO-d₆) δ 9.39 (d, J=1.3 Hz, 1H), 9.19 (s, 1H), 8.09 (d, J=9.3 Hz, 1H), 8.02 (d, J=3.1 Hz, 1H), 7.92 (dd, J=12.5, 1.4 Hz, 1H), 7.40 (d, J=9.4 Hz, 1H), 3.74 (dd, J=11.1, 5.9 Hz, 1H), 3.66 (d, J=9.8 Hz, 2H), 3.47 (s, 2H), 2.38 (d, J=8.2 Hz, 6H), 2.15 (dd, J=12.7, 5.8 Hz, 1H), 1.92 (s, 1H).

### Example 189: Synthesis of Compound 506

### Synthesis of Intermediate C224

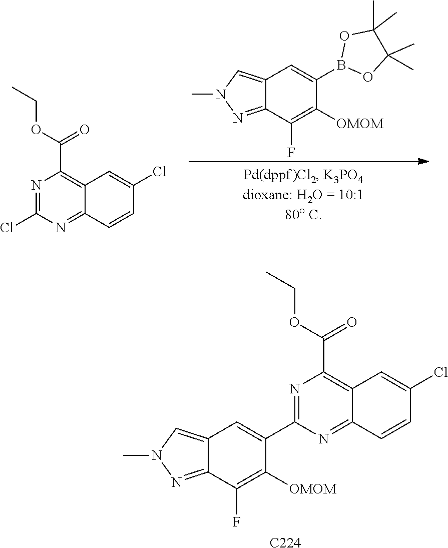

To a mixture of ethyl 2,6-dichloroquinazoline-4-carboxylate (1.5 g, 5.533 mmol, 1.0 equiv) and 7-fluoro-6-(methoxymethoxy)-2-methyl-5-(4,4,5,5-tetramethyl-1,3,2-dioxaborolan-2-yl) indazole (1.8 g, 5.533 mmol, 1.0 equiv) in dioxane (20 mL) and water (2 mL) was added K₃PO₄ (2.3 g, 11.066 mmol, 2.0 equiv) and Pd(dppf)Cl₂ (0.4 g, 0.553 mmol, 0.1 equiv). The reaction mixture was stirred for 3 h at 80° C. under a nitrogen atmosphere, then concentrated under reduced pressure to give a residue. The residue was purified by silica gel column chromatography, eluted with PE/EA (3:1) to afford ethyl 6-chloro-2-[7-fluoro-6-(methoxymethoxy)-2-methylindazol-5-yl]quinazoline-4-carboxylate (1.4 g, 52%) as a solid. LCMS (ES, m/z): 445 [M+H]⁺.

### Synthesis of Intermediate C225

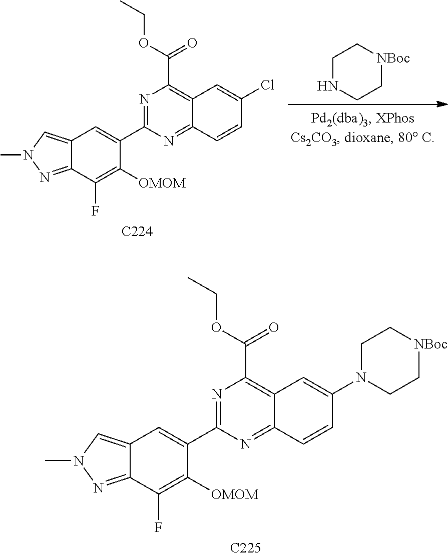

To a mixture of ethyl 6-chloro-2-[7-fluoro-6-(methoxymethoxy)-2-methylindazol-5-yl]quinazoline-4-carboxylate (330.0 mg, 0.742 mmol, 1.0 equiv) and tert-butyl piperazine-1-carboxylate (179.6 mg, 0.965 mmol, 1.3 equiv) in dioxane (8 mL) was added Cs₂CO₃ (483.4 mg, 1.484 mmol, 2.0 equiv), Pd₂(dba)₃ (67.9 mg, 0.074 mmol, 0.1 equiv), and XPhos (70.73 mg, 0.148 mmol, 0.2 equiv). The reaction mixture was stirred for 3 h at 80° C. under a nitrogen atmosphere, then concentrated under reduced pressure to give a residue. The residue was purified by silica gel column chromatography, eluted with PE/EA (3:1) to afford ethyl 6-[4-(tert-butoxycarbonyl)piperazin-1-yl]-2-[7-fluoro-6-(methoxymethoxy)-2-methylindazol-5-yl]quinazoline-4-carboxylate (320 mg, 65%) as a solid. LCMS (ES, m/z): 595 [M+H]⁺.

### Synthesis of Intermediate C226

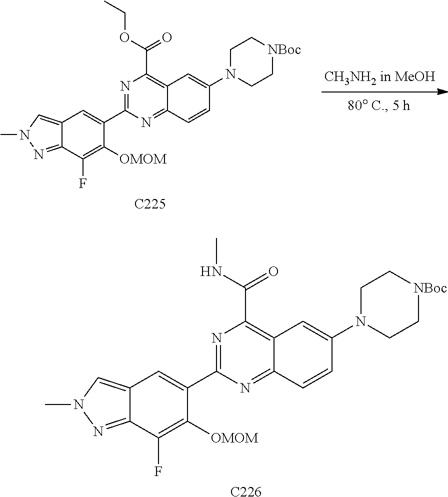

A mixture of ethyl 6-[4-(tert-butoxycarbonyl)piperazin-1-yl]-2-[7-fluoro-6-(methoxymethoxy)-2-methylindazol-5-yl]quinazoline-4-carboxylate (300.0 mg, 0.505 mmol, 1.0 equiv) and CH₃NH₂ in methanol (5 mL, 2M) was stirred for 5 h at 80° C. The resulting mixture was concentrated under reduced pressure to afford tert-butyl 4-{2-[7-fluoro-6-(methoxymethoxy)-2-methylindazol-5-yl]-4-(methylcarbamoyl)quinazolin-6-yl}piperazine-1-carboxylate (300 mg, 87%) as a solid. LCMS (ES, m/z): 580 [M+H]⁺.

### Synthesis of Compound 506

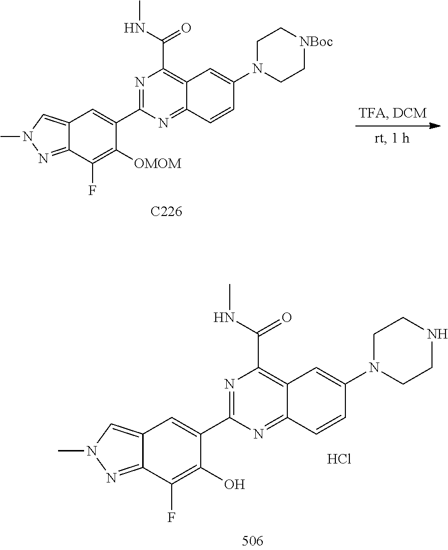

To a stirred solution of tert-butyl 4-{2-[7-fluoro-6-(methoxymethoxy)-2-methylindazol-5-yl]-4-(methylcarbamoyl)quinazolin-6-yl}piperazine-1-carboxylate (150.0 mg, 0.259 mmol, 1.0 equiv) in DCM (2 mL) was added TFA (0.3 mL) dropwise at room temperature. The resulting mixture was stirred for 1 h at room temperature, then concentrated under reduced pressure to give a residue. The residue was purified by Prep-HPLC (Condition 8, Gradient 1) to afford 2-(7-fluoro-6-hydroxy-2-methylindazol-5-yl)-N-methyl-6-(piperazin-1-yl)quinazoline-4-carboxamide (40 mg, 35%) as a solid. LCMS (ES, m/z): 436 [M+H]⁺. ¹H NMR (400 MHz, DMSO-d₆) δ 13.36 (s, 1H), 9.28 (d, J=5.1 Hz, 1H), 9.14 (s, 2H), 9.08 (s, 1H), 8.59 (d, J=2.6 Hz, 1H), 8.26 (d, J=2.7 Hz, 1H), 8.12 (d, J=9.3 Hz, 1H), 8.04 (dd, J=9.4, 2.8 Hz, 1H), 4.20 (s, 3H), 3.63-3.56 (m, 4H), 3.32-3.31 (m, 4H), 2.97 (d, J=4.4 Hz, 3H).

### Example 190: Synthesis of Compound 667

### Synthesis of Intermediate C227

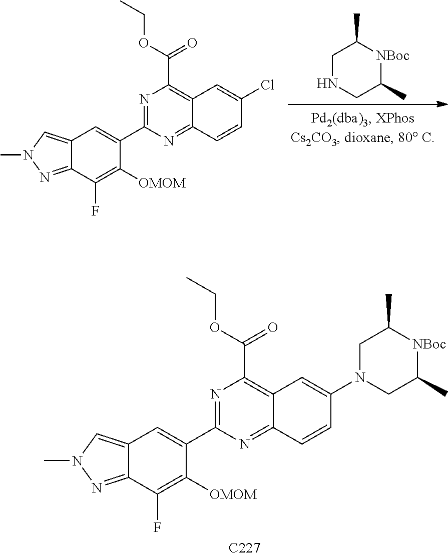

To a mixture of ethyl 6-chloro-2-[7-fluoro-6-(methoxymethoxy)-2-methylindazol-5-yl]quinazoline-4-carboxylate (330.0 mg, 0.742 mmol, 1.0 equiv) and tert-butyl (2R,6S)-2,6-dimethylpiperazine-1-carboxylate (206.6 mg, 0.965 mmol, 1.3 equiv) in dioxane (8 mL) was added Cs₂CO₃ (483.4 mg, 1.484 mmol, 2.0 equiv), Pd₂(dba)₃ (67.9 mg, 0.074 mmol, 0.1 equiv), and XPhos (70.7 mg, 0.148 mmol, 0.2 equiv). The reaction mixture was stirred for 3 h at 80° C. under a nitrogen atmosphere, then concentrated under reduced pressure to give a residue. The residue was purified by silica gel column chromatography, eluted with PE/EA (5:1) to afford ethyl 6-[(3R,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (350 mg, 69%) as a solid. LCMS (ES, m/z): 623 [M+H]⁺.

### Synthesis of Intermediate C228

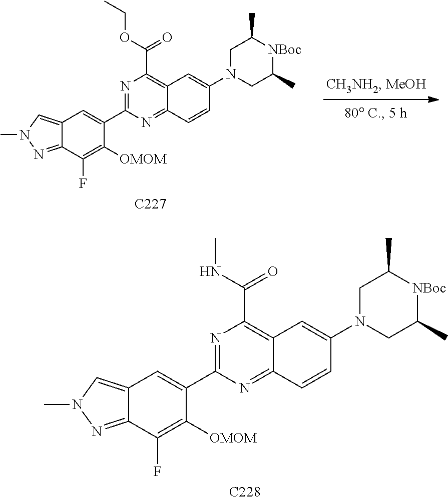

A mixture of ethyl 6-[(3R,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (330.0 mg, 0.530 mmol, 1.0 equiv) and methylamine (2M in methanol) (5 mL) was stirred for 5 h at 80° C. The resulting mixture was concentrated under reduced pressure to afford tert-butyl (2R,6S)-4-{2-[7-fluoro-6-(methoxymethoxy)-2-methylindazol-5-yl]-4-(methylcarbamoyl)quinazolin-6-yl}-2,6-dimethylpiperazine-1-carboxylate (320 mg, 84%) as a solid. LCMS (ES, m/z): 608 [M+H]⁺.

### Synthesis of Compound 667

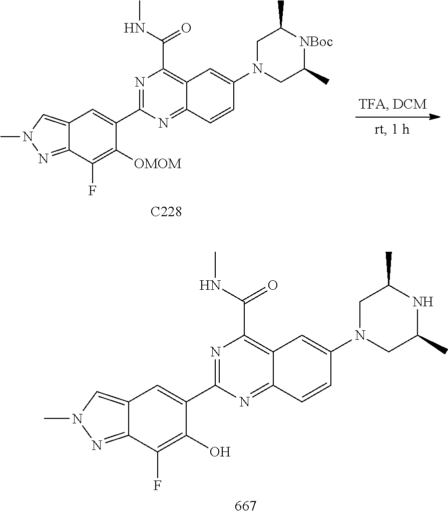

To a stirred solution of tert-butyl (2R,6S)-4-{2-[7-fluoro-6-(methoxymethoxy)-2-methylindazol-5-yl]-4-(methylcarbamoyl)quinazolin-6-yl}-2,6-dimethylpiperazine-1-carboxylate (150.0 mg, 0.247 mmol, 1.0 equiv) in DCM (2 mL) was added TFA (0.5 mL) dropwise at room temperature. The resulting mixture was stirred for 1 h at room temperature, then concentrated under reduced pressure to give a residue. The residue was purified by Prep-HPLC (Condition 15, Gradient 1) to afford 6-[(3R,5S)-3,5-dimethylpiperazin-1-yl]-2-(7-fluoro-6-hydroxy-2-methylindazol-5-yl)-N-methylquinazoline-4-carboxamide (53 mg, 46%) as a solid. LCMS (ES, m/z): 464 [M+H]⁺. 1H NMR (400 MHz, DMSO-d₆) δ 13.42 (s, 1H), 9.23 (d, J=5.1 Hz, 1H), 9.04 (d, J=0.9 Hz, 1H), 8.57 (d, J=2.5 Hz, 1H), 8.17 (t, J=1.7 Hz, 1H), 8.01 (d, J=1.6 Hz, 2H), 4.19 (s, 3H), 3.77 (dd, J=11.9, 2.8 Hz, 2H), 2.97 (d, J=4.6 Hz, 3H), 2.90 (ddd, J=9.8, 6.1, 2.9 Hz, 2H), 2.34 (t, J=11.1 Hz, 2H), 1.09 (d, J=6.3 Hz, 6H).

### Example 191: Synthesis of Compound 528

### Synthesis of Intermediate C229

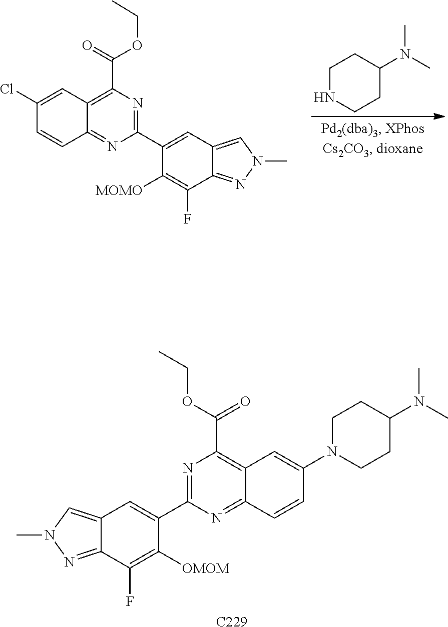

To a stirred mixture of ethyl 6-chloro-2-[7-fluoro-6-(methoxymethoxy)-2-methylindazol-5-yl]quinazoline-4-carboxylate (300 mg, 0.674 mmol, 1 equiv) and N,N-dimethylpiperidin-4-amine (103 mg, 0.809 mmol, 1.2 equiv) in 1,4-dioxane (3 mL) was added Cs₂CO₃ (659 mg, 2.022 mmol, 3 equiv), RuPhos (62 mg, 0.135 mmol, 0.2 equiv), and Pd₂(dba)₃ (61 mg, 0.067 mmol, 0.1 equiv) at room temperature under nitrogen atmosphere. The resulting mixture was stirred for 2 h at 100° C., then cooled to room temperature. The resulting mixture was concentrated under reduced pressure to give a residue. The residue was purified by silica gel column chromatography, eluted with PE/EA (1:1) to afford ethyl 6-[4-(dimethylamino)piperidin-1-yl]-2-[7-fluoro-6-(methoxymethoxy)-2-methylindazol-5-yl]quinazoline-4-carboxylate (300 mg, 83%) as a solid. LCMS (ES, m/z): 537 [M+H]⁺.

### Synthesis of Intermediate C230

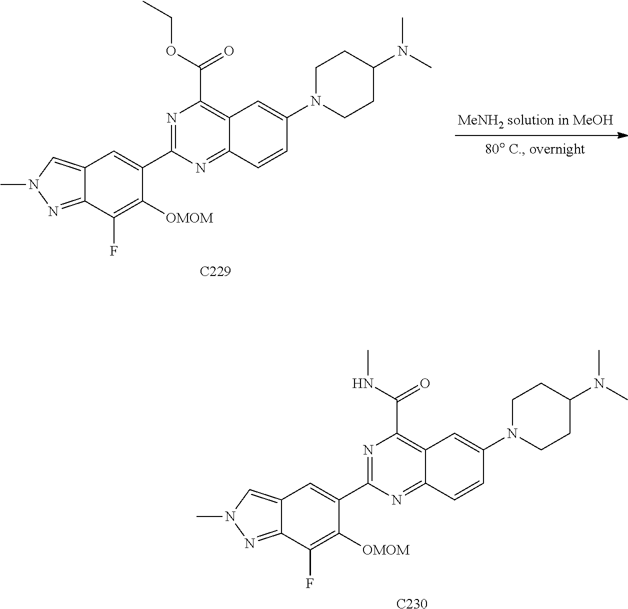

A mixture of ethyl 6-[4-(dimethylamino)piperidin-1-yl]-2-[7-fluoro-6-(methoxymethoxy)-2-methylindazol-5-yl]quinazoline-4-carboxylate (300 mg, 0.559 mmol, 1 equiv) and MeNH₂ solution in methanol (5 mL) was stirred for 16 h at 80° C. The mixture was cooled to room temperature, then concentrated under reduced pressure to give a residue. The residue was purified by silica gel column chromatography, eluted with PE/EA (1:1) to afford 6-[4-(dimethylamino)piperidin-1-yl]-2-[7-fluoro-6-(methoxymethoxy)-2-methylindazol-5-yl]-N-methylquinazoline-4-carboxamide (250 mg, 86%) as a solid. LCMS (ES, m/z): 522 [M+H]⁺.

### Synthesis of Compound 528

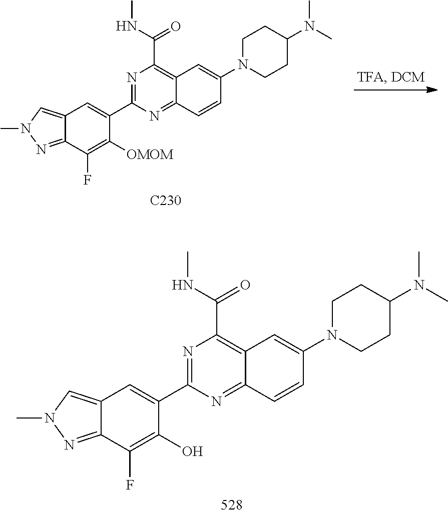

To a stirred solution of 6-[4-(dimethylamino)piperidin-1-yl]-2-[7-fluoro-6-(methoxymethoxy)-2-methylindazol-5-yl]-N-methylquinazoline-4-carboxamide (250 mg, 0.479 mmol, 1 equiv) in DCM (3 mL) was added TFA (1 mL) at room temperature. The resulting mixture was stirred for 1 h at room temperature, then concentrated under reduced pressure to give a residue. The residue was purified by reverse flash chromatography (Condition 10, Gradient 10) to afford 6-[4-(dimethylamino)piperidin-1-yl]-2-(7-fluoro-6-hydroxy-2-methylindazol-5-yl)-N-methylquinazoline-4-carboxamide (50 mg, 22%) as a solid. LCMS (ES, m/z): 478 [M+H]⁺. ¹H NMR (300 MHz, DMSO-d₆) δ 13.43 (s, 1H), 9.22 (d, J=5.0 Hz, 1H), 9.04 (d, J=1.0 Hz, 1H), 8.57 (d, J=2.6 Hz, 1H), 8.17 (d, J=1.7 Hz, 1H), 8.00 (d, J=1.5 Hz, 2H), 4.19 (s, 3H), 3.94 (d, J=12.6 Hz, 2H), 3.00-2.85 (m, 5H), 2.33 (s, 1H), 2.22 (s, 6H), 1.96-1.86 (m, 2H), 1.51 (q, J=10.8 Hz, 2H).

### Example 192: Synthesis of Compound 529

### Synthesis of Intermediate C231

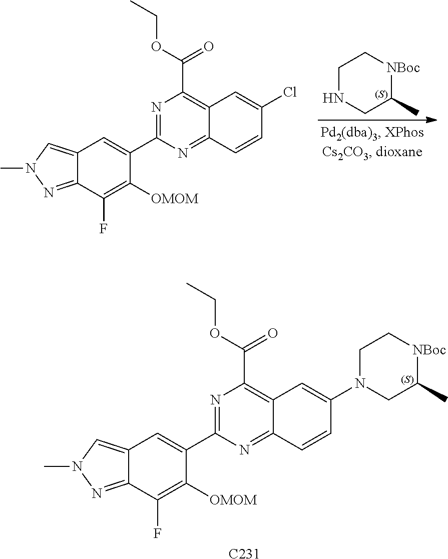

To a stirred mixture of ethyl 6-chloro-2-[7-fluoro-6-(methoxymethoxy)-2-methylindazol-5-yl]quinazoline-4-carboxylate (320 mg, 0.719 mmol, 1 equiv) and tert-butyl (2S)-2-methylpiperazine-1-carboxylate (172 mg, 0.863 mmol, 1.2 equiv) in 1,4-dioxane (3 mL) was added Cs₂CO₃ (703 mg, 2.157 mmol, 3 equiv), RuPhos (67 mg, 0.144 mmol, 0.2 equiv), and Pd₂(dba)₃ (65 mg, 0.072 mmol, 0.1 equiv) at room temperature under nitrogen atmosphere. The resulting mixture was stirred for 2 h at 100° C., then cooled to room temperature and concentrated under reduced pressure to give a residue. The residue was purified by silica gel column chromatography, eluted with PE/EA (1:1) to afford ethyl 6-[(3S)-4-(tert-butoxycarbonyl)-3-methylpiperazin-1-yl]-2-[7-fluoro-6-(methoxymethoxy)-2-methylindazol-5-yl]quinazoline-4-carboxylate (340 mg, 78%) as a solid. LCMS (ES, m/z): 609 [M+H]⁺.

### Synthesis of Intermediate C232

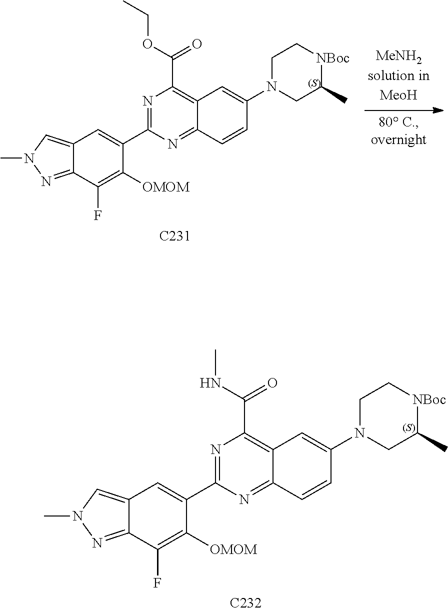

A mixture of ethyl 6-[(3S)-4-(tert-butoxycarbonyl)-3-methylpiperazin-1-yl]-2-[7-fluoro-6-(methoxymethoxy)-2-methylindazol-5-yl]quinazoline-4-carboxylate (300 mg, 0.493 mmol, 1 equiv) and MeNH₂ in methanol (3 mL) was stirred for overnight at 80° C. The reaction mixture was cooled to room temperature, then concentrated under reduced pressure to give a residue. The residue was purified by silica gel column chromatography, eluted with PE/EA (1:1) to afford tert-butyl (2S)-4-{2-[7-fluoro-6-(methoxymethoxy)-2-methylindazol-5-yl]-4-(methylcarbamoyl)quinazolin-6-yl}-2-methylpiperazine-1-carboxylate (260 mg, 89%) as a solid. LCMS (ES, m/z): 594 [M+H]⁺.

### Synthesis of Compound 529

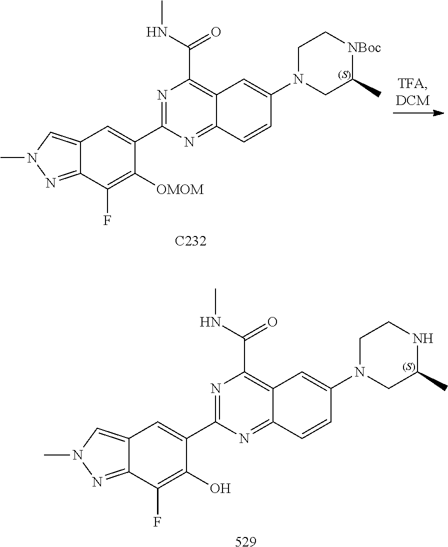

To a stirred solution of tert-butyl (2S)-4-{2-[7-fluoro-6-(methoxymethoxy)-2-methylindazol-5-yl]-4-(methylcarbamoyl)quinazolin-6-yl}-2-methylpiperazine-1-carboxylate (260 mg, 0.438 mmol, 1 equiv) in DCM (3 mL) was added TFA (1 mL) at room temperature. The resulting mixture was stirred for 1 h at room temperature, then concentrated under reduced pressure to give a residue. The residue was purified by reverse flash chromatography (Condition 10, Gradient 10) to afford 2-(7-fluoro-6-hydroxy-2-methylindazol-5-yl)-N-methyl-6-[(3S)-3-methylpiperazin-1-yl]quinazoline-4-carboxamide (60 mg, 30%) as a solid. LCMS (ES, m/z): 450 [M+H]⁺. ¹H NMR (300 MHz, DMSO-d₆) δ 13.44 (s, 1H), 9.23 (d, J=5.2 Hz, 1H), 9.05 (d, J=1.1 Hz, 1H), 8.58 (d, J=2.7 Hz, 1H), 8.16 (s, 1H), 8.02 (s, 2H), 4.19 (s, 3H), 3.78 (d, J=10.1 Hz, 2H), 3.05 (d, J=10.6 Hz, 1H), 2.97 (d, J=4.7 Hz, 3H), 2.80 (t, J=12.1 Hz, 3H), 2.42 (t, J=10.7 Hz, 1H), 1.09 (d, J=6.2 Hz, 3H).

### Example 193: Synthesis of Compound 385

Into a 20 mL vial were added 2-chloro-6-[6-(methoxymethoxy)-2-methylindazol-5-yl]-1,5-naphthyridine (150 mg, 0.423 mmol, 1 equiv), tert-butyl N-cyclopropyl-N-(pyrrolidin-3-yl)carbamate (114.82 mg, 0.508 mmol, 1.2 equiv), DMSO (4 mL, 56.314 mmol, 133.20 equiv) and DIEA (163.93 mg, 1.269 mmol, 3 equiv) at room temperature. The resulting mixture was stirred for overnight at 100° C. The mixture was allowed to cool down to room temperature. The reaction was quenched by the addition of Water (10 mL) at room temperature. The resulting mixture was extracted with EtOAc (3×5 mL). The combined organic layers were washed with subsaturation brine (3×5 mL), dried over anhydrous Na₂SO₄. After filtration, the filtrate was concentrated under reduced pressure. The residue was purified by silica gel column chromatography, eluted with CH₂Cl₂/EA (1:4) to afford tert-butyl N-cyclopropyl-N-(1-{6-[6-(methoxymethoxy)-2-methylindazol-5-yl]-1,5-naphthyridin-2-yl}pyrrolidin-3-yl)carbamate (130 mg, 56.45%) as a Brown yellow oil.

LCMS (ES, m/z): 545 [M+H]+

### Example 194: Synthesis of Compound 386

### Synthesis of Intermediate D2

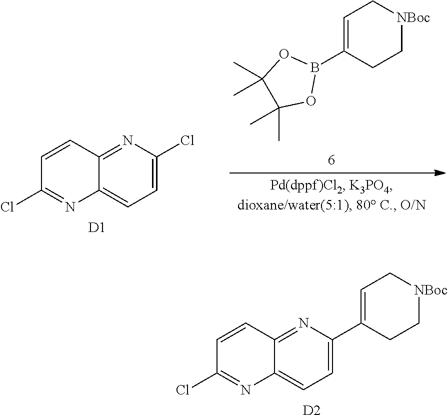

To a stirred mixture of 2,6-dichloro-1,5-naphthyridine (D1, 1 g, 5.024 mmol, 1 equiv) and tert-butyl 4-(4,4,5,5-tetramethyl-1,3,2-dioxaborolan-2-yl)-3,6-dihydro-2H-pyridine-1-carboxylate (1.55 g, 5.024 mmol, 1 equiv) and Pd(dppf)Cl₂·CH₂Cl₂ (0.28 g, 0.502 mmol, 0.1 equiv) in 1,4-dioxane (20 mL) was added K₃PO₄ (3.20 g, 15.072 mmol, 3 equiv) in water (4 mL) dropwise at room temperature under nitrogen atmosphere. The resulting mixture was stirred overnight at 80° C. under nitrogen atmosphere. The mixture was allowed to cool to room temperature, then diluted with water (60 mL) and extracted with EtOAc (3×60 mL). The combined organic layers were washed with brine (1×50 mL) and subsequently dried over anhydrous Na₂SO₄. After filtration, the filtrate was concentrated under reduced pressure and purified by silica gel column chromatography and eluted with CH₂Cl₂/EtOAc (1:1) to afford tert-butyl 4-(6-chloro-1,5-naphthyridin-2-yl)-3,6-dihydro-2H-pyridine-1-carboxylate (D2, 800 mg). LCMS: (ESI, m/z): 346[M+H].

### Synthesis of Intermediate D3

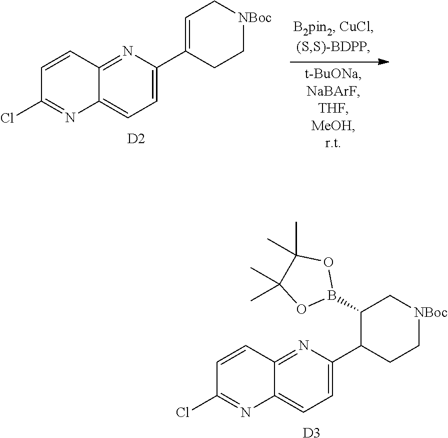

A mixture of CuCl (34.35 mg, 0.347 mmol, 0.3 equiv) and (2S,4S)-(−)-2,4-Bis(diphenylphosphino)pentane (101.90 mg, 0.231 mmol, 0.2 equiv) and sodium 2-methylpropan-2-olate (222.31 mg, 2.314 mmol, 2 equiv) and sodium tetrakis[3,5-bis(trifluoromethyl)phenyl]borate (205 mg, 0.231 mmol, 0.2 equiv) in THE (15 mL) was stirred for 10 min at room temperature under nitrogen atmosphere. To the above mixture was added 4,4,5,5-tetramethyl-2-(tetramethyl-1,3,2-dioxaborolan-2-yl)-1,3,2-dioxaborolane (881.15 mg, 3.471 mmol, 3 equiv) (in 7.5 mL THF) dropwise over 10 min at 0° C. The resulting mixture was stirred for additional 10 min at room temperature. To the above mixture was added Intermediate D2 (400 mg, 1.157 mmol, 1 equiv) in THF (7.5 mL)/MeOH (10 mL)) dropwise over 15 min at 0° C. The resulting mixture was stirred for an additional 3 days at room temperature, quenched by the addition of water/ice (50 mL) at 0° C. and extracted with CH₂Cl₂ (3×50 mL). The combined organic layers were washed with brine (1×50 mL), dried over anhydrous Na₂SO₄, and the filtrate was concentrated under reduced pressure. The residue was subsequently purified by reverse flash chromatography, Condition 3, Gradient 6 as previously described to afford (3S)-4-(6-chloro-1,5-naphthyridin-2-yl)-3-(4,4,5,5-tetramethyl-1,3,2-dioxaborolan-2-yl) piperidine-1-carboxylate (D3, 200 mg). LCMS: (ESI, m/z): 474[M+H].

### Synthesis of Intermediate D4

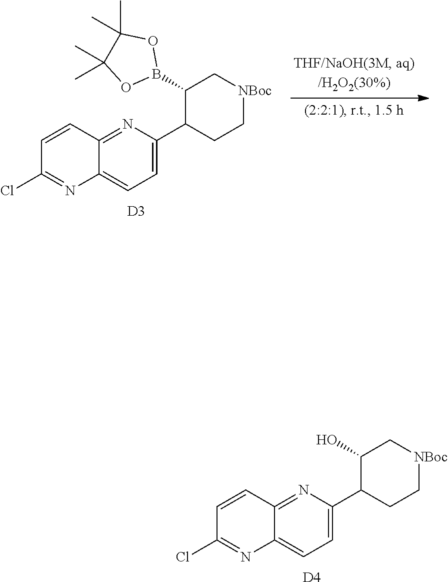

To a stirred mixture of Intermediate D3 (200 mg, 0.422 mmol, 1 equiv) in THE (4 mL) was added NaOH (3M, aq) (4 mL) was added dropwise to a stirred mixture of Intermediate D3 (200 mg, 0.422 mmol, 1 equiv) in THE (4 mL) at 0° C. To the above mixture was added H₂O₂(30%) (2 mL) dropwise at 0° C., and the resulting mixture was stirred for an additional 1.5 h at room temperature. The reaction was quenched by the addition of sat. Na₂S₂O₃ (aq.) (20 mL) at 0° C. and subsequently extracted with EtOAc (3×30 mL). The combined organic layers were washed with brine (1×30 mL), dried over anhydrous Na₂SO₄, and the filtrate was concentrated under reduced pressure. The residue was purified by reverse flash chromatography following Condition 3, Gradient 7 as previously described. Further, the residue was purified by silica gel column chromatography and eluted with DCE/EA (2:1) to afford tert-butyl (3S)-4-(6-chloro-1,5-naphthyridin-2-yl)-3-hydroxypiperidine-1-carboxylate (D4, 75 mg). LCMS: (ESI, m/z): 364[M+H].

### Synthesis of Intermediate D5

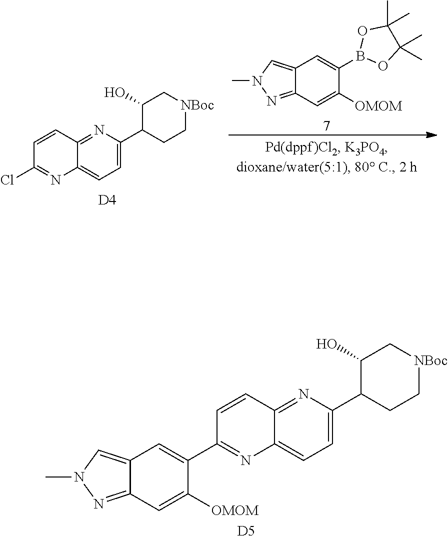

A mixture of Intermediate D4 (65.59 mg, 0.206 mmol, 1 equiv), Pd(dppf)Cl₂·CH₂Cl₂ (11.39 mg, 0.021 mmol, 0.1 equiv) and K₃PO₄ (131.27 mg, 0.618 mmol, 3 equiv) in 1,4-dioxane (1.5 mL)/water (0.5 mL) was stirred for 2 h at 80° C. under nitrogen atmosphere and later allowed to cool to room temperature. The resulting mixture was diluted with water (10 mL) and extracted with EtOAc (3×10 mL). The combined organic layers were washed with brine (1×10 mL), dried over anhydrous Na₂SO₄, and the filtrate was concentrated under reduced pressure. The residue was purified by silica gel column chromatography and eluted with DCM/EA (1:1) to afford tert-butyl (3S)-3-hydroxy-4-{6-[6-(methoxymethoxy)-2-methylindazol-5-yl]-1,5-naphthyridin-2-yl}piperidine-1-carboxylate (D5, 50 mg). LCMS: (ESI, m/z): 520[M+H].

### Synthesis of Compound 386

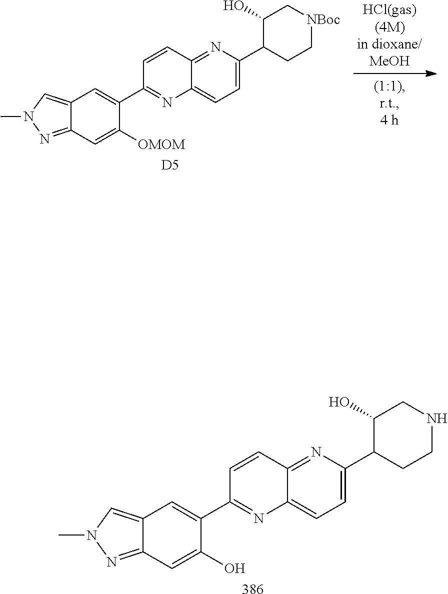

To a stirred mixture of Intermediate D5 (50 mg, 0.096 mmol, 1 equiv) in MeOH (2.5 mL) was added HCl(gas) in 1,4-dioxane (2.5 mL) dropwise at room temperature under air atmosphere. The resulting mixture was stirred for 4 h at room temperature under air atmosphere and later concentrated under reduced pressure. The crude product was purified by Prep-HPLC following Condition 2, Gradient 1 as previously described to afford 5-{6-[(3S)-3-hydroxypiperidin-4-yl]-1,5-naphthyridin-2-yl}-2-methylindazol-6-ol (Compound 386, 10.7 mg). Absolute stereochemistry of the compound was arbitrarily assigned. LCMS: (ESI, m/z): 376[M+H]. ¹H NMR: (400 MHz, DMSO-d₆): δ 13.65 (s, 1H), 8.70 (s, 1H), 8.61 (d, J=9.3 Hz, 1H), 8.49 (d, J=9.1 Hz, 1H), 8.41 (d, J=7.4 Hz, 2H), 7.81 (d, J=8.8 Hz, 1H), 6.92 (s, 1H), 4.72 (d, J=5.2 Hz, 1H), 4.13 (s, 4H), 3.18 (dt, J=12.6, 3.1 Hz, 1H), 3.10 (d, J=13.3 Hz, 1H), 2.97 (dd, J=13.4, 2.8 Hz, 1H), 2.82 (dd, J=13.2, 1.7 Hz, 1H), 2.63 (dd, J=12.7, 2.8 Hz, 1H), 2.20 (qd, J=12.6, 4.2 Hz, 1H), 1.70 (d, J=12.6 Hz, 1H).

### Example 195: Synthesis of Compound 387

### Synthesis of Intermediate D7

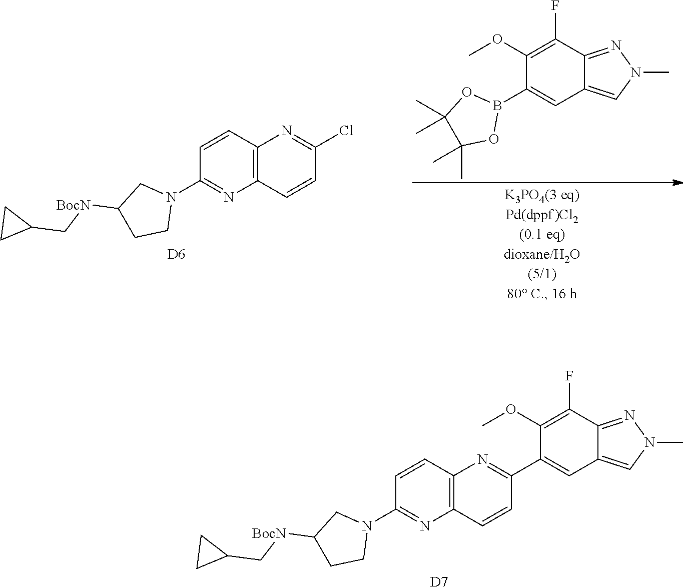

A solution of tert-butyl N-[1-(6-chloro-1,5-naphthyridin-2-yl)pyrrolidin-3-yl]-N-(cyclopropylmethyl)carbamate (D6, 500 mg, 1.241 mmol, 1 equiv), 7-fluoro-6-methoxy-2-methyl-5-(4,4,5,5-tetramethyl-1,3,2-dioxaborolan-2-yl) indazole (380 mg, 1.241 mmol, 1 equiv), Pd(DtBPF)Cl₂ (81 mg, 0.124 mmol, 0.1 equiv) and K₃PO₄ (790 mg, 3.723 mmol, 3 equiv) in dioxane (10 mL) and H₂O (2 mL) was stirred for 16 h at 80° C. under nitrogen atmosphere and was subsequently allowed to cool to room temperature. The resulting mixture was poured into water (20 mL), extracted with EA (3×20 mL) and dried over anhydrous Na₂SO₄. After filtration, the filtrate was concentrated under reduced pressure. The residue was purified by silica gel column chromatography and eluted with PE/EA (1:1) to afford tert-butyl N-(cyclopropylmethyl)-N-{1-[6-(7-fluoro-6-methoxy-2-methylindazol-5-yl)-1,5-naphthyridin-2-yl]pyrrolidin-3-yl}carbamate (D7, 300 mg). LCMS: (ES, m/z):547 [M+H]⁺

### Synthesis of Intermediate D8

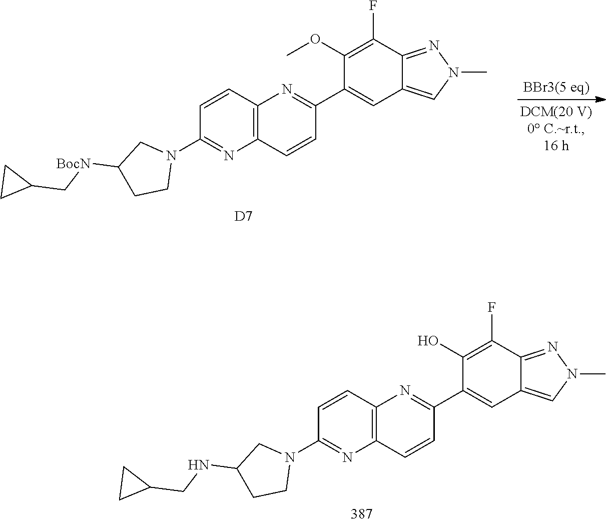 [SMILES: COc1c(-c2ccc3nc(N4CCC(N(CC5CC5)C(=O)OC(C)(C)C)C4)ccc3n2)cc2cn(C)nc2c1F ; "D7" ; Cn1cc2cc(-c3ccc4nc(N5CCC(NCC6CC6)C5)ccc4n3)c(O)c(F)c2n1 ; "387"]

To a stirred solution of Intermediate D7 (300 mg, 0.549 mmol, 1 equiv) in DCM (6 mL) was added BBr₃ (687 mg, 2.745 mmol, 5 equiv) dropwise at 0° C., and the resulting mixture was stirred for 16 h at room temperature. The reaction was quenched by the addition of MeOH (30 mL) at 0° C. and concentrated under reduced pressure. The mixture was then basified to pH 8 with saturated NaHCO₃ (aq.), extracted with CH₂Cl₂ (3×50 mL) and dried over anhydrous Na₂SO₄. After filtration, the filtrate was concentrated under reduced pressure. The crude product was purified by Chiral-Prep-HPLC following Condition 9, Gradient 1 as previously described to afford 5-(6-{3-[(cyclopropylmethyl)amino]pyrrolidin-1-yl}-1,5-naphthyridin-2-yl)-7-fluoro-2-methylindazol-6-ol (75 mg) as a yellow solid. 5-(6-{3-[(cyclopropylmethyl)amino]pyrrolidin-1-yl}-1,5-naphthyridin-2-yl)-7-fluoro-2-methylindazol-6-ol (D8, 13.2 mg) to QC. LCMS: (ES, m/z):433 [M+H]⁺. ¹H NMR: (400 MHz, DMSO-d₆) δ 14.44 (s, 1H), 8.48 (d, J=2.7 Hz, 1H), 8.44-8.37 (m, 2H), 8.14 (dd, J=9.3, 0.8 Hz, 1H), 8.11-8.05 (m, 1H), 7.17 (d, J=9.3 Hz, 1H), 4.16 (s, 3H), 3.82-3.68 (m, 3H), 3.57 (d, J=8.8 Hz, 1H), 3.45 (t, J=5.4 Hz, 1H), 2.46 (d, J=6.6 Hz, 2H), 2.13 (dq, J=12.9, 6.6 Hz, 1H), 1.88 (dd, J=12.3, 6.2 Hz, 2H), 0.96-0.82 (m, 1H), 0.46-0.35 (m, 2H), 0.17-0.05 (m, 2H).

### Example 196: Synthesis of Compound 390

### Synthesis of Intermediate D8

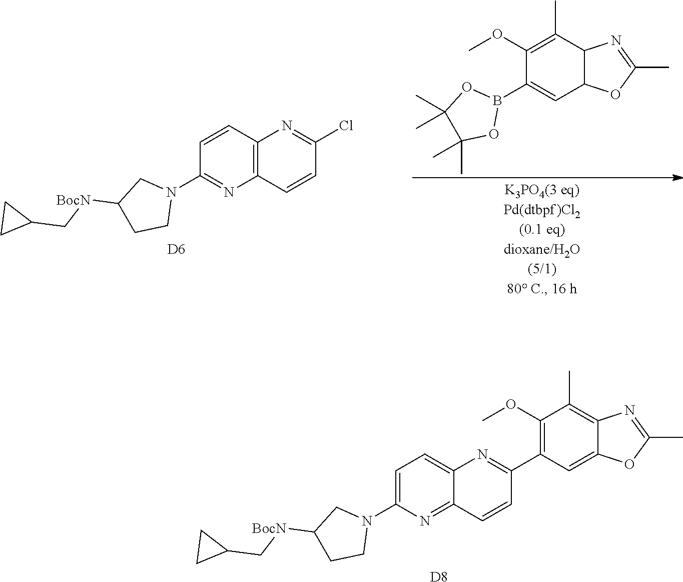

A solution of Intermediate D6 (500 mg, 1.241 mmol, 1 equiv), 5-methoxy-2,4-dimethyl-6-(4,4,5,5-tetramethyl-1,3,2-dioxaborolan-2-yl)-1,3-benzoxazole (376 mg, 1.241 mmol, 1 equiv), Pd(DtBPF)Cl₂ (81 mg, 0.124 mmol, 0.1 equiv) and K₃PO₄ (790 mg, 3.723 mmol, 3 equiv) in dioxane (10 mL) and H₂O (2 mL) was stirred for 16 h at 80° C. under nitrogen atmosphere and later allowed to cool to room temperature. The resulting mixture was poured into water (20 mL), extracted with EA (3×20 mL), dried over anhydrous Na₂SO₄, and the filtrate was concentrated under reduced pressure. The residue was purified by silica gel column chromatography and eluted with PE/EA (1:1) to afford tert-butyl N-(cyclopropylmethyl)-N-{1-[6-(5-methoxy-2,4-dimethyl-1,3-benzoxazol-6-yl)-1,5-naphthyridin-2-yl]pyrrolidin-3-yl}carbamate (Intermediate D8, 360 mg). LCMS: (ES, m/z):544 [M+H]⁺.

### Synthesis of Compound 390

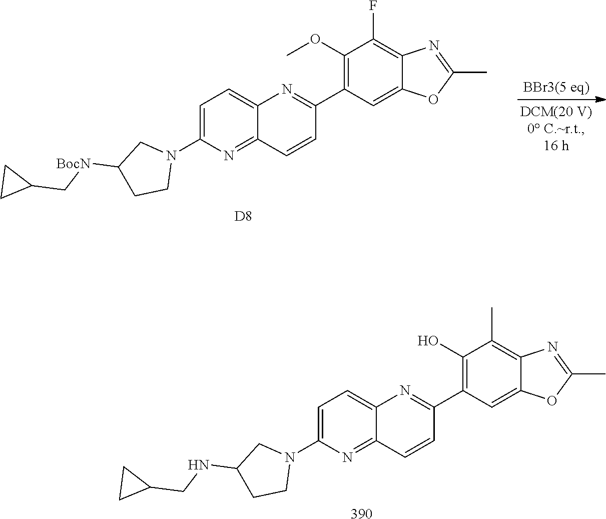 [SMILES: COc1c(-c2ccc3nc(N4CCC(N(CC5CC5)C(=O)OC(C)(C)C)C4)ccc3n2)cc2oc(C)nc2c1F ; "D8" ; Cc1nc2c(C)c(O)c(-c3ccc4nc(N5CCC(NCC6CC6)C5)ccc4n3)cc2o1 ; "390"]

To a stirred solution of Intermediate D8 (360 mg, 0.662 mmol, 1 equiv) in DCM (7.2 mL) was added BBr₃ (829 mg, 3.310 mmol, 5.00 equiv) dropwise at 0° C., and the resulting mixture was stirred for 16 h at room temperature. The reaction was quenched by the addition of MeOH (30 mL) at 0° C. and concentrated under reduced pressure. The mixture was then basified to pH 8 with saturated NaHCO₃ (aq.), extracted with CH₂Cl₂ (3×50 mL), dried over anhydrous Na₂SO₄, and the filtrate was concentrated under reduced pressure. The crude product was purified by Chiral-Prep-HPLC following Condition 9, Gradient 1 as previously described to afford 6-(6-{3-[(cyclopropylmethyl)amino]pyrrolidin-1-yl}-1,5-naphthyridin-2-yl)-2,4-dimethyl-1,3-benzoxazol-5-ol (140 mg). 6-(6-{3-[(cyclopropylmethyl)amino]pyrrolidin-1-yl}-1,5-naphthyridin-2-yl)-2,4-dimethyl-1,3-benzoxazol-5-ol (Compound 390, 14.7 mg) to QC. LCMS: (ES, m/z):430 [M+H]⁺. ¹H NMR: (400 MHz, DMSO-d₆) δ 14.99 (s, 1H), 8.41 (d, J=9.3 Hz, 1H), 8.25 (s, 1H), 8.14 (dd, J=9.2, 0.8 Hz, 1H), 8.07 (dd, J=9.1, 0.8 Hz, 1H), 7.16 (d, J=9.3 Hz, 1H), 3.74 (dd, J=10.5, 5.8 Hz, 1H), 3.68 (s, 1H), 3.60-3.51 (m, 1H), 3.45 (t, J=5.4 Hz, 1H), 3.41-3.30 (s, 1H), 2.61 (s, 3H), 2.46 (d, J=6.8 Hz, 2H), 2.40 (s, 3H), 2.13 (dq, J=12.9, 6.5 Hz, 1H), 1.87 (dd, J=12.4, 6.4 Hz, 1H), 0.95-0.84 (m, 1H), 0.46-0.33 (m, 2H), 0.17-0.09 (m, 2H).

### Example 197: Synthesis of Compound 287

### Synthesis of Intermediate D10

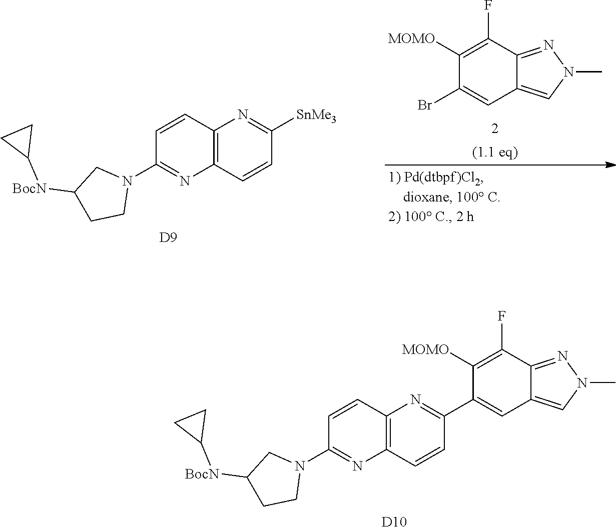

A mixture of 5-bromo-7-fluoro-6-(methoxymethoxy)-2-methylindazole (296.45 mg, 1.026 mmol, 1.2 equiv) and Pd(DtBPF)Cl₂ (55.69 mg, 0.086 mmol, 0.1 equiv) in 1,4-dioxane (8 mL) was stirred for 45 min at 100° C. under nitrogen atmosphere. To the above mixture was added tert-butyl N-cyclopropyl-N-{1-[6-(trimethylstannyl)-1,5-naphthyridin-2-yl]pyrrolidin-3-yl}carbamate (D9, 680 mg, 0.855 mmol, 1 equiv, 65%) (in 1,4-dioxane (5.6 mL)) dropwise over 10 min at 100° C., and the resulting mixture was stirred for additional 2 h at 100° C. and then allowed to cool down to room temperature. The resulting mixture was concentrated under reduced pressure, purified by silica gel column chromatography, and eluted with CH₂Cl₂/MeOH (10:1) to afford tert-butyl N-cyclopropyl-N-(1-{6-[7-fluoro-6-(methoxymethoxy)-2-methylindazol-5-yl]-1,5-naphthyridin-2-yl}pyrrolidin-3-yl)carbamate (525 mg). The crude product (525 mg) was purified by reverse flash chromatography following Condition 3, Gradient 3 as previously described, resulting in tert-butyl N-cyclopropyl-N-(1-{6-[7-fluoro-6-(methoxymethoxy)-2-methylindazol-5-yl]-1,5-naphthyridin-2-yl}pyrrolidin-3-yl)carbamate (170 mg) and tert-butyl N-cyclopropyl-N-(1-{6-[7-fluoro-6-(methoxymethoxy)-2-methylindazol-5-yl]-1,5-naphthyridin-2-yl}pyrrolidin-3-yl)carbamate (125 mg). The crude product (125 mg) was purified by Prep-HPLC following Condition 1, Gradient 32 as previously described to afford tert-butyl N-cyclopropyl-N-(1-{6-[7-fluoro-6-(methoxymethoxy)-2-methylindazol-5-yl]-1,5-naphthyridin-2-yl}pyrrolidin-3-yl)carbamate (D10, 75 mg). LCMS: (ESI, m/z): 563[M+H].

### Synthesis of Compound 287

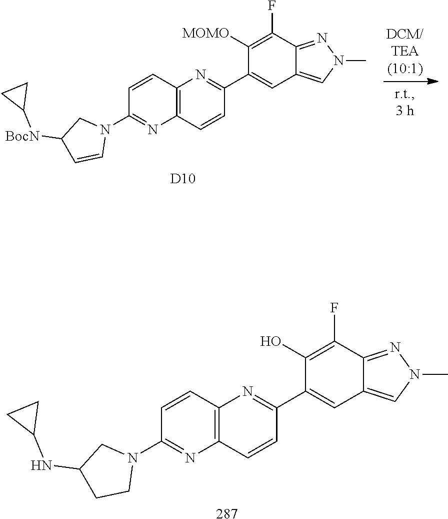

To a stirred solution of Intermediate D10 (245 mg, 0.435 mmol, 1 equiv) in DCM (12 mL) was added TFA (1.2 mL) dropwise at 0° C., and the resulting mixture was subsequently stirred for 3 h at room temperature and later concentrated under vacuum. The crude product was purified by Prep-HPLC following Condition 1, Gradient 32 as previously described to afford 5-{6-[3-(cyclopropylamino)pyrrolidin-1-yl]-1,5-naphthyridin-2-yl}-7-fluoro-2-methylindazol-6-ol; trifluoroacetic acid (Compound 287, 170 mg) LCMS: (ESI, m/z): 419[M+H-TFA]. ¹H-NMR: (400 MHz, DMSO-d₆): δ 14.44 (s, 1H), 8.48 (d, J=2.6 Hz, 1H), 8.43-8.36 (m, 2H), 8.13 (d, J=9.3 Hz, 1H), 8.07 (d, J=9.1 Hz, 1H), 7.15 (d, J=9.3 Hz, 1H), 4.16 (s, 3H), 3.76 (dd, J 10.9, 5.9 Hz, 1H), 3.67 (s, 1H), 3.57 (t, J=5.6 Hz, 2H), 3.45 (s, 1H), 2.26-2.11 (m, 2H), δ 2.04-1.88 (m, 1H), 0.53-0.40 (m, 2H), 0.33 (q, J=6.7, 4.2 Hz, 2H).

### Example 198: Synthesis of Compound 305 and Compound 306

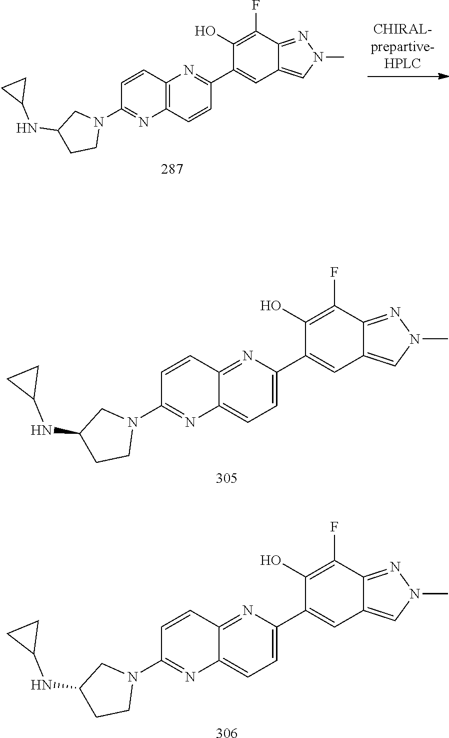 [SMILES: Cn1cc2cc(-c3ccc4nc(N5CCC(NC6CC6)C5)ccc4n3)c(O)c(F)c2n1 ; "287" ; Cn1cc2cc(-c3ccc4nc(N5CC[C@@H](NC6CC6)C5)ccc4n3)c(O)c(F)c2n1 ; "305" ; Cn1cc2cc(-c3ccc4nc(N5CC[C@H](NC6CC6)C5)ccc4n3)c(O)c(F)c2n1 ; "306"]

The racemic Compound 287 (157.5 mg, 0.296 mmol, 1 equiv) was dissolved in DCM (5 mL), basified to pH 7 with NH₃(7 M) in MeOH, diluted with water (30 mL), and extracted with CH₂Cl₂ (3×30 mL). The combined organic layers were washed with water (1×10 mL), dried over anhydrous Na₂SO₄, and the filtrate was concentrated under reduced pressure. This resulted in the solid racemate (Compound 287, 126 mg). The racemate (126 mg) was purified by Chiral-Prep-HPLC following Condition 4, Gradient 10 as previously described. The product was dissolved in hexane (5 mL), and the precipitated solids were collected by filtration and washed with hexane (3×1 mL), resulting in resolution of the diastereomers. The stereochemistry was assigned arbitrarily. This resulted in 5-{6-[(3R)-3-(cyclopropylamino)pyrrolidin-1-yl]-1,5-naphthyridin-2-yl}-7-fluoro-2-methylindazol-6-ol (Compound 305, 35.4 mg). Compound 305 was resolved in the second peak. LCMS: (ESI, m/z): 419[M+H]. ¹H-NMR: (400 MHz, DMSO-d₆): δ 14.44 (s, 1H), 8.48 (d, J=2.7 Hz, 1H), 8.44-8.37 (m, 2H), 8.11 (dd, J=21.4, 9.2 Hz, 2H), 7.17 (d, J=9.3 Hz, 1H), 4.16 (s, 3H), 3.75 (dd, J=10.9, 5.8 Hz, 1H), 3.67 (s, 1H), 3.62-3.48 (m, 2H), 3.43 (s, 1H), 2.18-2.10 (m, 2H), 1.94 (s, 1H), 0.43 (d, J=6.7 Hz, 2H), 0.30-0.21 (m, 2H). Compound 306, its diastereomer was resolved in the first peak. 5-{6-[(3S)-3-(cyclopropylamino)pyrrolidin-1-yl]-1,5-naphthyridin-2-yl}-7-fluoro-2-methylindazol-6-ol (Compound 306, 39.7 mg). LCMS: (ESI, m/z): 419[M+H]. ¹H-NMR: (400 MHz, DMSO-d₆): δ 14.45 (s, 1H), 8.48 (d, J=2.7 Hz, 1H), 8.40 (t, J=4.6 Hz, 2H), 8.10 (dd, J=20.8, 9.2 Hz, 2H), 7.16 (d, J=9.3 Hz, 1H), 4.16 (s, 3H), 3.75 (dd, J=10.8, 5.9 Hz, 1H), 3.70-3.62 (m, 1H), 3.61-3.47 (m, 2H), 3.42 (s, 1H), 2.15 (tt, J=6.6, 3.3 Hz, 2H), 1.98-1.90 (m, 1H), 0.42 (t, J=6.7 Hz, 2H), 0.34-0.21 (m, J=5.7 Hz, 2H).

### Example 199: Synthesis of Compound 288

### Synthesis of Intermediate D12

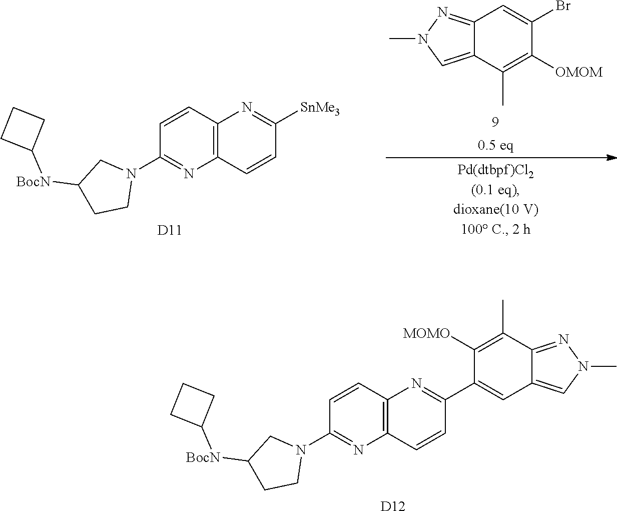

A solution of 5-bromo-6-(methoxymethoxy)-2,7-dimethylindazole (225 mg, 0.789 mmol, 1 equiv) and tert-butyl N-cyclobutyl-N-{1-[6-(trimethylstannyl)-1,5-naphthyridin-2-yl]pyrrolidin-3-yl}carbamate (D11, 629 mg, 1.183 mmol, 1.5 equiv), Pd(DtBPF)Cl₂ (51 mg, 0.079 mmol, 0.1 equiv) in 1,4-dioxane (2.25 mL) was stirred for 2 h at 100° C. under nitrogen atmosphere and later allowed to cool to room temperature. The resulting mixture was extracted with EA (1×10 mL), dried over anhydrous Na₂SO₄, and the filtrate was concentrated under reduced pressure. The residue was purified by silica gel column chromatography and eluted with PE/EA (1:1) to afford tert-butyl N-cyclobutyl-N-(1-{6-[6-(methoxymethoxy)-2,7-dimethylindazol-5-yl]-1,5-naphthyridin-2-yl}pyrrolidin-3-yl)carbamate (D12, 149 mg). LCMS: (ES, m/z): 573 [M+H]⁺.

### Synthesis of Compound 288

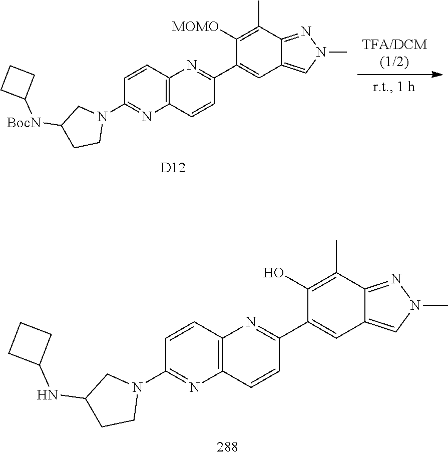

A solution of Intermediate D12 (149 mg, 0.260 mmol, 1 equiv) in DCM (2 mL) and CF₃COOH (1 mL) was stirred for 1 h at room temperature and subsequently concentrated under reduced pressure. The crude product was then purified by Prep-HPLC following Condition 1, Gradient 33 as previously described to afford 5-{6-[3-(cyclobutylamino)pyrrolidin-1-yl]-1,5-naphthyridin-2-yl}-2,7-dimethylindazol-6-ol (Compound 288, 8.4 mg). LCMS: (ES, m/z): 429 [M+H]⁺. 1H NMR: (400 MHz, DMSO-d₆) δ 14.33 (s, 1H), 8.43-8.36 (m, 2H), 8.33 (s, 1H), 8.06 (d, J=9.1 Hz, 2H), 7.14 (d, J=9.3 Hz, 1H), 4.13 (s, 3H), 3.69 (s, 2H), 3.68 (s, 1H), 3.55 (s, 1H), 3.25 (s, 2H), 2.38 (s, 3H), 2.14 (s, 4H), 2.18-2.05 (m, 1H), 1.82 (s, 2H), 1.75-1.50 (m, 2H).

### Example 200: Synthesis of Compound 289

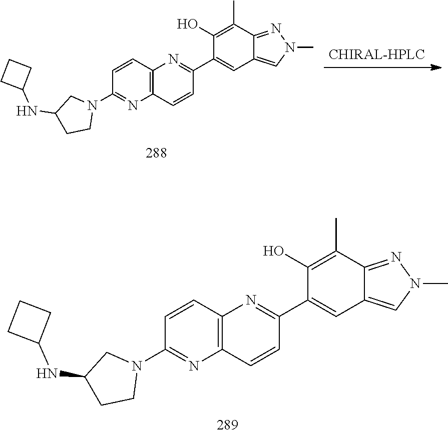 [SMILES: Cc1c(O)c(-c2ccc3nc(N4CCC(NC5CCC5)C4)ccc3n2)cc2cn(C)nc12 ; Cc1c(O)c(-c2ccc3nc(N4CC[C@@H](NC5CCC5)C4)ccc3n2)cc2cn(C)nc12]

The crude product 288 was purified by preparative chiral HPLC following Condition 10, Gradient 1 as previously described to afford the resolved diastereomers. Stereochemistry was assigned arbitrarily. 5-{6-[(3R)-3-(cyclobutylamino)pyrrolidin-1-yl]-1,5-naphthyridin-2-yl}-2,7-dimethylindazol-6-ol (Compound 289, 22.3 mg). LCMS: (ES, m/z): 429 [M+H]⁺. ¹H NMR: (400 MHz, DMSO-d₆) δ 14.32 (s, 1H), 8.43-8.36 (m, 2H), 8.33 (s, 1H), 8.12 (d, J=9.3 Hz, 1H), 8.06 (d, J=9.1 Hz, 1H), 7.14 (d, J=9.2 Hz, 1H), 4.13 (s, 3H), 3.69 (s, 2H), 3.55 (s, 1H), 3.38 (s, 2H), 3.23 (s, 1H), 2.38 (s, 3H), 2.14 (s, 4H), 1.83 (s, 1H), 1.71 (s, 2H), 1.57 (dt, J=18.3, 8.8 Hz, 2H). 5-{6-[(3S)-3-(cyclobutylamino)pyrrolidin-1-yl]-1,5-naphthyridin-2-yl}-2,7-dimethylindazol-6-ol (Compound 290, 23.3 mg). LCMS: (ES, m/z): 429 [M+H]⁺. ¹H NMR: (400 MHz, DMSO-d₆) δ 14.32 (s, 1H), 8.43-8.36 (m, 2H), 8.33 (s, 1H), 8.13 (d, J=9.2 Hz, 1H), 8.06 (d, J=9.1 Hz, 1H), 7.15 (d, J=9.3 Hz, 1H), 4.13 (s, 3H), 3.70 (d, J=7.8 Hz, 2H), 3.55 (d, J=8.4 Hz, 1H), 3.38 (s, 2H), 3.23 (s, 1H), 2.38 (s, 3H), 2.13 (s, 4H), 1.84 (s, 1H), 1.72 (s, 2H), 1.58 (dt, J=18.7, 9.0 Hz, 2H).

### Example 201: Synthesis of Compounds 291 and 292

### Synthesis of Intermediate D14

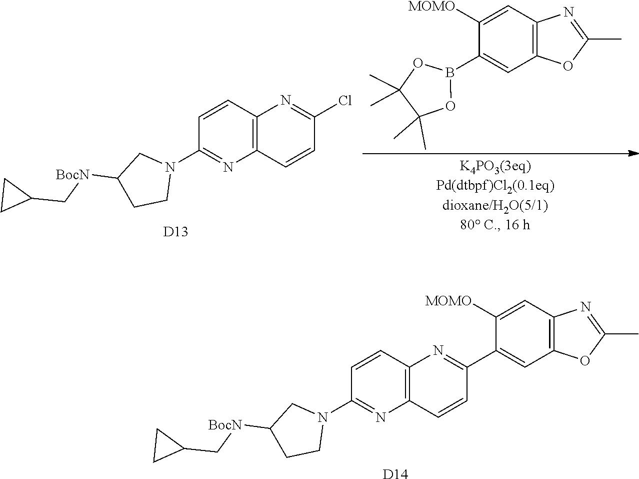

A solution of tert-butyl N-[1-(6-chloro-1,5-naphthyridin-2-yl)pyrrolidin-3-yl]-N-(cyclopropylmethyl)carbamate (D13, 500 mg, 1.241 mmol, 1 equiv), 5-(methoxymethoxy)-2-methyl-6-(4,4,5,5-tetramethyl-1,3,2-dioxaborolan-2-yl)-1,3-benzoxazole (396 mg, 1.241 mmol, 1.00 equiv), Pd(DtBPF)Cl₂ (81 mg, 0.124 mmol, 0.1 equiv) and K₃PO₄ (790 mg, 3.723 mmol, 3 equiv) in dioxane (10 mL) and H₂O (2 mL) was stirred for 16 h at 80° C. under nitrogen atmosphere, and later allowed to cool to room temperature. The resulting mixture was poured into water (20 mL), extracted with EA (3×20 mL), dried over anhydrous Na₂SO₄, and the filtrate was concentrated under reduced pressure. The residue was purified by silica gel column chromatography and eluted with PE/EA (1:1) to afford tert-butyl N-(cyclopropylmethyl)-N-(1-{6-[5-(methoxymethoxy)-2-methyl-1,3-benzoxazol-6-yl]-1,5-naphthyridin-2-yl}pyrrolidin-3-yl)carbamate (D14, 350 mg). LCMS: (ES, m/z):560 [M+H]⁺

### Synthesis of Intermediate D15

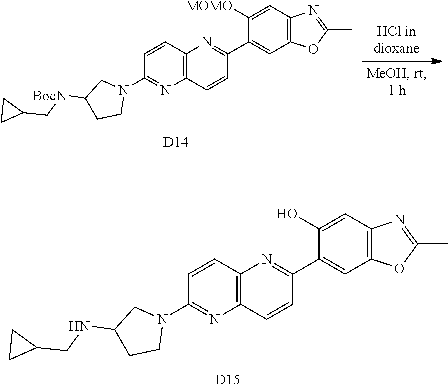

A solution of Intermediate D14 (350 mg, 0.625 mmol, 1 equiv) in HCl(gas) in 1,4-dioxane (1 mL) and MeOH (2 mL) was stirred for 1 h at room temperature and then concentrated under reduced pressure. The crude product was purified by Preparative chiral HPLC following Condition 11, Gradient 1 to afford 6-(6-{3-[(cyclopropylmethyl)amino]pyrrolidin-1-yl}-1,5-naphthyridin-2-yl)-2-methyl-1,3-benzoxazol-5-ol (D15, 15.5 mg) to QC. LCMS: (ES, m/z): 416 [M+H]⁺. ¹H NMR: (400 MHz, DMSO-d₆) δ 14.53 (s, 1H), 8.45-8.39 (m, 2H), 8.10 (dd, J=15.2, 9.2 Hz, 2H), 7.16 (d, J=9.3 Hz, 1H), 7.12 (s, 1H), 3.74 (dd, J=10.8, 5.9 Hz, 1H), 3.68 (s, 1H), 3.58 (s, 1H), 3.48-3.41 (m, 1H), 2.61 (s, 3H), 2.46 (d, J=6.7 Hz, 2H), 2.13 (dq, J=12.8, 6.6 Hz, 1H), 1.88-1.85 (m, 2H), 0.96-0.82 (m, 1H), 0.45-0.37 (m, 2H), 0.17-0.05 (m, 2H).

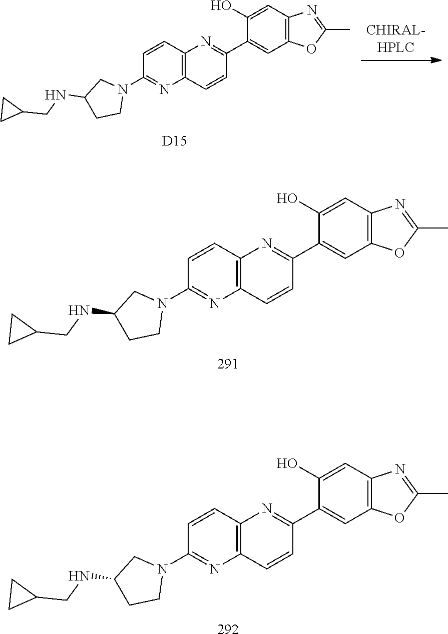

The intermediate D15 (90 mg, 0.217 mmol, 1 equiv) was purified by preparative chiral HPLC following Condition 4, Gradient 11 to afford Compound 291 (First peak, 17.7 mg) and Compound 292 (Second peak, 18.5 mg). Absolute stereochemistry of the chiral compounds was assigned arbitrarily. Compound 291 (First peak): LCMS: (ES, m/z): 416 [M+H]⁺. ¹H NMR: (400 MHz, DMSO-d₆) δ 14.53 (s, 1H), 8.46-8.39 (m, 2H), 8.11 (dd, J=17.2, 9.2 Hz, 2H), 7.17 (d, J=9.3 Hz, 1H), 7.12 (s, 1H), 3.75 (dd, J=9.9, 5.4 Hz, 1H), 3.68 (s, 1H), 3.58 (d, J=8.0 Hz, 1H), 3.57-3.48 (m, 1H), 3.47-3.38 (m, 1H), 2.61 (s, 3H), 2.48 (s, 2H), 2.20-2.10 (m, 1H), 1.89-1.88 (m, 1H), 0.95-0.85 (m, 1H), 0.47-0.38 (m, 2H), 0.19-0.10 (m, 2H). Compound 292 (Second peak). LCMS: (ES, m/z): 416 [M+H]⁺. ¹H NMR: (400 MHz, DMSO-d₆) δ 14.54 (s, 1H), 8.46-8.39 (m, 2H), 8.16-8.05 (m, 2H), 7.17 (d, J=9.3 Hz, 1H), 7.12 (s, 1H), 3.78-3.66 (m, 2H), 3.58 (d, J=8.0 Hz, 1H), 3.57-3.48 (m, 1H), 3.47-3.38 (m, 1H), 2.61 (s, 3H), 2.47 (d, J=6.4 Hz, 2H), 2.14 (dd, J=12.5, 6.3 Hz, 1H), 1.89-1.88 (m, 1H), 0.94-0.84 (m, 1H), 0.46-0.37 (m, 2H), 0.18-0.10 (m, 2H).

### Example 202: Synthesis of Compound 932

### Synthesis of Intermediate D17

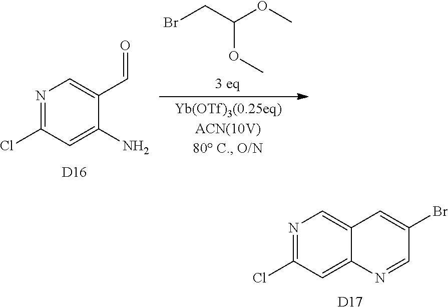

A solution of 4-amino-6-chloropyridine-3-carbaldehyde (D16, 5 g, 31.935 mmol, 1 equiv), Yb(OTf)₃ (4.95 g, 7.984 mmol, 0.25 equiv), and 2-bromo-1,1-dimethoxy-ethane, (16.19 g, 95.805 mmol, 3 equiv) in ACN (50 mL) was stirred for 16 h at 0-80° C. under nitrogen atmosphere, and later allowed to cool to room temperature. The precipitated solids were collected by filtration, washed with ACN (1×50 mL), and the residue was purified by silica gel column chromatography and eluted with PE/EA (5:1) to afford 3-bromo-7-chloro-1,6-naphthyridine (D17, 2 g). LCMS: (ES, m/z): 243 [M+H]⁺

### Synthesis of Intermediate D18

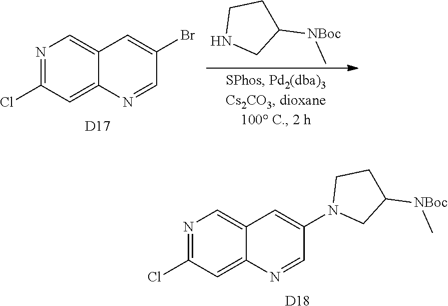

A solution of Intermediate D17 (300 mg, 1.232 mmol, 1 equiv), tert-butyl N-methyl-N-(pyrrolidin-3-yl)carbamate (296 mg, 1.478 mmol, 1.2 equiv), SPhos (101 mg, 0.246 mmol, 0.2 equiv), Pd₂(dba)₃ (113 mg, 0.123 mmol, 0.1 equiv), and Cs₂CO₃ (1204 mg, 3.696 mmol, 3 equiv) in 1,4-dioxane (6 mL) was stirred for 2 h at 100° C. under nitrogen atmosphere and allowed to cool to room temperature. The resulting mixture was extracted with EA (1×30 mL), dried over anhydrous Na₂SO₄, and the filtrate was concentrated under reduced pressure. The residue was purified by silica gel column chromatography and eluted with PE/EA (1:1) to afford tert-butyl N-[1-(7-chloro-1,6-naphthyridin-3-yl)pyrrolidin-3-yl]-N-methylcarbamate (D18, 300 mg). LCMS: (ES, m/z): 363 [M+H]⁺.

### Synthesis of Intermediate D19

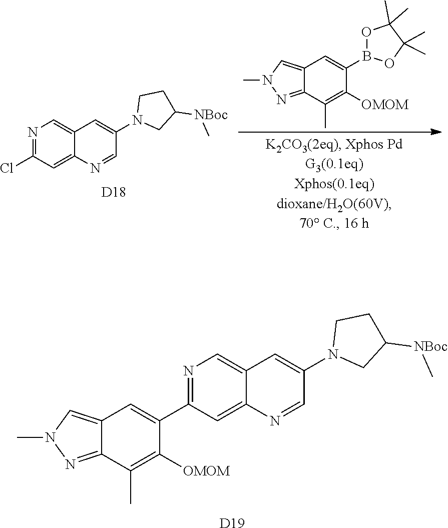

To a stirred solution of Intermediate D18 (90 mg, 0.248 mmol, 1 equiv) and K₂CO₃ (69 mg, 0.496 mmol, 2 equiv), XPhos (12 mg, 0.025 mmol, 0.1 equiv), XPhos Pd G₃ (21 mg, 0.025 mmol, 0.1 equiv) in 1,4-dioxane (4.5 mL) and water (0.9 mL) was added 6-(methoxymethoxy)-2,7-dimethyl-5-(4,4,5,5-tetramethyl-1,3,2-dioxaborolan-2-yl) indazole (99 mg, 0.298 mmol, 1.2 equiv) in portions at 70° C. under nitrogen atmosphere and the resulting mixture was stirred for 16 h at 70° C. and subsequently allowed to cool to room temperature. The resulting mixture was extracted with EA (1×20 mL), dried over anhydrous Na₂SO₄, and the filtrate was concentrated under reduced pressure. The residue was purified by silica gel column chromatography and eluted with PE/EA (1:1) to afford tert-butyl N-(1-{7-[6-(methoxymethoxy)-2,7-dimethylindazol-5-yl]-1,6-naphthyridin-3-yl}pyrrolidin-3-yl)-N-methylcarbamate (D19, 35 mg). LCMS: (ES, m/z): 533 [M+H]⁺.

### Synthesis of Compound 932

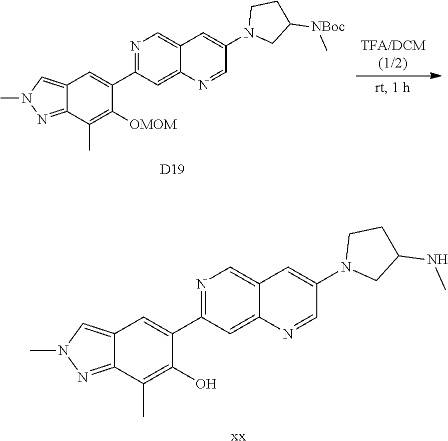

A solution of Intermediate D19 (35 mg, 0.066 mmol, 1 equiv) in DCM (1 mL) and CF₃COOH (0.5 mL) was stirred for 1 h at room temperature. The resulting mixture was concentrated under reduced pressure, and the crude product was purified by Prep-HPLC following Condition 3, Gradient 15 as previously described to afford 2,7-dimethyl-5-{3-[3-(methylamino)pyrrolidin-1-yl]-1,6-naphthyridin-7-yl}indazol-6-ol (Compound 932, 15.9 mg). LCMS: (ES, m/z): 389 [M+H]⁺. ¹H NMR: (400 MHz, DMSO-d₆) δ 13.66 (s, 1H), 9.20 (s, 1H), 8.86 (d, J=2.9 Hz, 1H), 8.48 (s, 1H), 8.34 (s, 1H), 8.27 (s, 1H), 7.27 (d, J=2.9 Hz, 1H), 4.13 (s, 3H), 3.63 (m, 1H), 3.54 (m, 1H), 3.48 (m, 1H), 3.29-3.21 (m, 1H), 2.36 (s, J=11.0 Hz, 3H), 2.34 (s, J=11.0 Hz, 3H), 2.16 (dq, J=13.1, 7.0 Hz, 1H), 1.90 (dt, J=11.9, 6.1 Hz, 2H).

### Example 203: Synthesis of Compounds 312, 313, and 314

### Synthesis of Intermediate D21

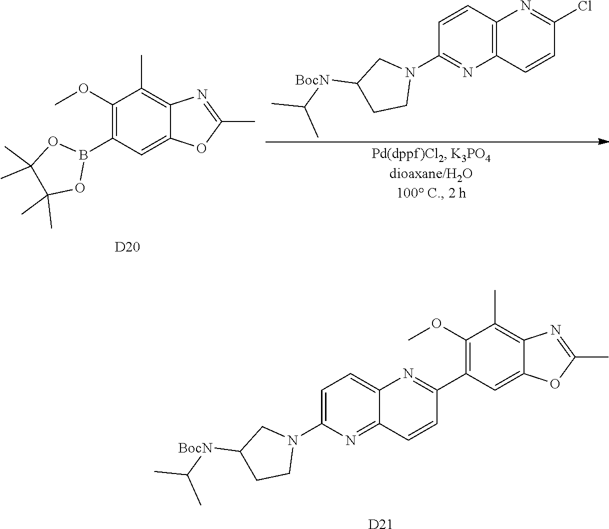

To a stirred solution of 5-methoxy-2,4-dimethyl-6-(4,4,5,5-tetramethyl-1,3,2-dioxaborolan-2-yl)-1,3-benzoxazole (D20, 300 mg, 0.990 mmol, 1 equiv) and tert-butyl N-[1-(6-chloro-1,5-naphthyridin-2-yl) pyrrolidin-3-yl]-N-isopropylcarbamate (580.23 mg, 1.485 mmol, 1.5 equiv) in dioxane (16 mL) and H₂O (4 mL) were added Pd(dppf)Cl₂CH₂Cl₂ (80.61 mg, 0.099 mmol, 0.1 equiv) and K₃PO₄ (630.14 mg, 2.970 mmol, 3 equiv) in portions at room temperature. The mixture was stirred for 2 h at 100° C. under nitrogen atmosphere and later allowed to cool to room temperature. The reaction was quenched with water and extracted with EtOAc (3×20 mL). The combined organic layers were washed with water (3×20 mL), dried over anhydrous Na₂SO₄, and the filtrate was concentrated under reduced pressure. The residue was purified by silica gel column chromatography and eluted with PE/EA (1:1) to afford tert-butyl N-isopropyl-N-{1-[6-(5-methoxy-2,4-dimethyl-1,3-benzoxazol-6-yl)-1,5-naphthyridin-2-yl]pyrrolidin-3-yl}carbamate (D21, 310 mg). LCMS: (ES, m/z): 532 [M+H].

### Synthesis of Compound 312

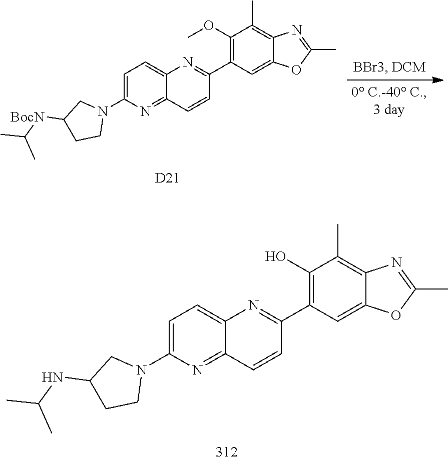

BBr3 (1319.38 mg, 5.270 mmol, 10 equiv) was added dropwise over 10 min at 0° C. to a 20 mL 4-necked round-bottom flask containing Intermediate D21 (280 mg, 0.527 mmol, 1 equiv) and DCM (6 mL) initially mixed at room temperature. The resulting mixture was stirred for additional three days at room temperature, quenched by the addition of MeOH (3 mL) at 0° C., and extracted with EtOAc (1×20 mL). The combined organic layers were washed with water (3×10 mL), dried over anhydrous Na2SO4, and the filtrate was concentrated under reduced pressure. The crude product was purified by Prep-HPLC following Condition 1, Gradient 29 as previously described to afford 6-{6-[3-(isopropylamino)pyrrolidin-1-yl]-1,5-naphthyridin-2-yl}-2,4-dimethyl-1,3-benzoxazol-5-ol (Compound 312, 45.2 mg). LCMS: (ES, m/z): 418 [M+H]. ¹H NMR: (400 MHz, DMSO-d₆) δ 14.99 (s, 1H), 8.41 (d, J=9.3 Hz, 1H), 8.25 (s, 1H), 8.13 (d, J=9.3 Hz, 1H), 8.07 (d, J=9.1 Hz, 1H), 7.16 (d, J=9.3 Hz, 1H), 3.78 (d, J=8.3 Hz, 1H), 3.70 (s, 1H) 3.52 (s, 2H), 3.32 (s, 1H), 2.85 (p, J=6.3 Hz, 1H), 2.61 (s, 3H), 2.40 (s, 3H), 2.15 (dt, J=12.4, 6.3 Hz, 1H), 1.79 (dt, J=14.1, 7.2 Hz, 1H), 1.72 (s, 1H), 1.02 (dd, J=6.2, 4.6 Hz, 6H).

### Synthesis of Compounds 313 and 314

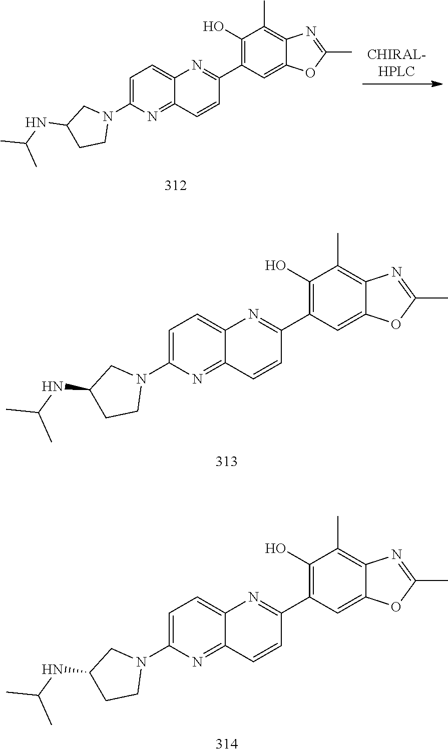

Compound 312 (110 mg, 0.263 mmol, 1 equiv) was purified by Preparative chiral HPLC following Condition 4, Gradient 12 as previously described to afford 6-{6-[(3S)-3-(isopropylamino)pyrrolidin-1-yl]-1,5-naphthyridin-2-yl}-2,4-dimethyl-1,3-benzoxazol-5-ol (Compound 313, 36.4 mg) and 6-{6-[(3R)-3-(isopropylamino)pyrrolidin-1-yl]-1,5-naphthyridin-2-yl}-2,4-dimethyl-1,3-benzoxazol-5-ol (Compound 314 36.8 mg). Absolute stereochemistry of the chiral compounds was assigned arbitrarily. Compound 313: (ES, m/z): 418 [M+H]. ¹H NMR: (400 MHz, DMSO-d₆) δ 14.99 (s, 1H), 8.42 (d, J=9.3 Hz, 1H), 8.26 (s, 1H), 8.14 (d, J=9.3 Hz, 1H), 8.08 (d, J=9.1 Hz, 1H), 7.17 (d, J=9.3 Hz, 1H), 3.79 (s, 1H), 3.71 (s, 1H), 3.53 (s, 2H), 3.33 (s, 1H), 2.90-2.82 (m, 1H), 2.61 (s, 3H), 2.40 (s, 3H), 2.16 (dd, J=11.6, 5.9 Hz, 1H), 1.81 (s, 2H), 1.02 (dd, J=6.2, 4.5 Hz, 6H). Compound 314: LCMS: (ES, m/z): 418 [M+H]. ¹H NMR: (400 MHz, DMSO-d₆) δ 14.99 (s, 1H), 8.41 (d, J=9.2 Hz, 1H), 8.25 (s, 1H), 8.14 (d, J=9.3 Hz, 1H), 8.08 (d, J=9.1 Hz, 1H), 7.17 (d, J=9.3 Hz, 1H), 3.79 (s, 1H), 3.70 (s, 1H), 3.53 (t, J=9.2 Hz, 2H), 3.33 (s, 1H), 2.87 (t, J=6.4 Hz, 1H), 2.61 (s, 3H), 2.40 (s, 3H), 2.16 (dd, J=11.9, 6.3 Hz, 1H), 1.81 (s, 2H), 1.03 (dd, J=6.2, 4.5 Hz, 6H).

### Example 204: Synthesis of Compound 501

### Synthesis of Intermediate D22

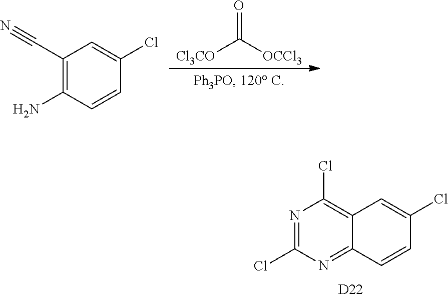

Into a 250 mL round bottom flask were added 2-amino-5-chlorobenzonitrile (5 g, 32.770 mmol, 1 equiv), triphenylphosphine oxide (0.91 g, 3.277 mmol, 0.1 equiv), chlorobenzene (120 mL) and triethylamine (0.03 g, 0.328 mmol, 0.01 equiv) at room temperature. The resulting mixture was stirred for 0.5 h at 0° C. before adding triphosgene (11.67 g, 39.324 mmol, 1.2 equiv) and stirring for an additional 5 h at 120° C. The reaction was quenched by the addition of water (200 mL) at room temperature, the aqueous layer was extracted with EtOAc (2×100 mL) and dried over Na₂SO₄. The resulting mixture was concentrated under reduced pressure, purified by silica gel column chromatography, and eluted with PE/EA (3:1) to afford 2,4,6-trichloroquinazoline (D22, 5.5 g). LCMS: (ES, m/z): 233 [M+H]⁺.

### Synthesis of Intermediate D23

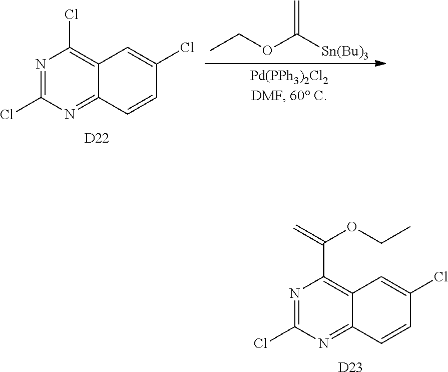

Into a 100 mL round bottom flask were added Intermediate D22 (2 g, 8.566 mmol, 1 equiv), DMF (40 mL), tributyl(1-ethoxyethenyl)stannane (3.71 g, 10.279 mmol, 1.2 equiv) and palladium chloride; bis(triphenylphosphine) (0.60 g, 0.857 mmol, 0.1 equiv) at room temperature. The resulting mixture was stirred for 3 h at 60° C. under nitrogen atmosphere and subsequently quenched by the addition of water (100 mL) at room temperature. The aqueous layer was extracted with EtOAc (2×100 mL) and dried over Na₂SO₄. The resulting mixture was concentrated under reduced pressure, purified by silica gel column chromatography, and eluted with PE/EA (3:1) to afford 2,6-dichloro-4-(1-ethoxyethenyl)quinazoline (D23, 1.7 g). LCMS: (ES, m/z): 269 [M+H]⁺.

### Synthesis of Intermediate D24

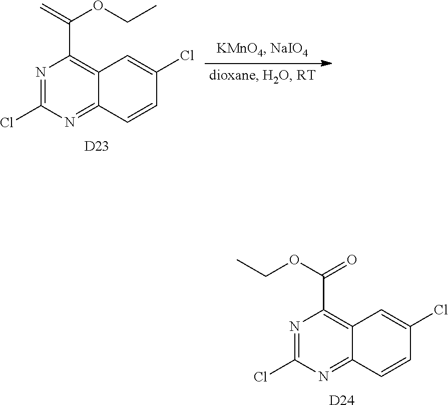

Into a 40 mL vial were added Intermediate D23 (1.6 g, 5.945 mmol, 1 equiv), dioxane (10 mL), water (10 mL), sodium periodate (1.27 g, 5.945 mmol, 1 equiv). and tetraoxo(potassio) manganese (1.88 g, 11.890 mmol, 2 equiv) at room temperature. The resulting mixture was stirred for 3 h at 25° C. and afterwards diluted with water (30 mL). The aqueous layer was extracted with EtOAc (2×100 mL) and dried over Na₂SO₄. The resulting mixture was concentrated under reduced pressure, the residue purified by silica gel column chromatography, and eluted with PE/EA (3:1) to afford ethyl 2,6-dichloroquinazoline-4-carboxylate (D24, 940 mg). LCMS: (ES, m/z): 271 [M+H]⁺.

### Synthesis of Intermediate D25

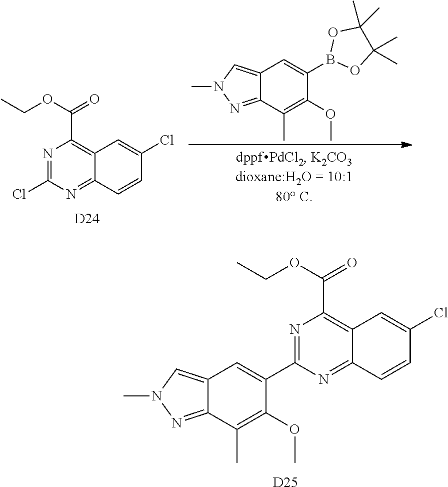

To a solution of Intermediate D24 (330 mg, 1.217 mmol, 1 equiv) and 6-methoxy-2,7-dimethyl-5-(4,4,5,5-tetramethyl-1,3,2-dioxaborolan-2-yl) indazole (441.40 mg, 1.460 mmol, 1.2 equiv) in dioxane (6 mL) and H₂O (0.5 mL) were added K₂CO₃ (504.70 mg, 3.651 mmol, 3 equiv) and Pd(dppf)Cl₂ (89.07 mg, 0.122 mmol, 0.10 equiv). After stirring for 6 h at 80° C. under a nitrogen atmosphere, it was concentrated under reduced pressure, the residue was purified by silica gel column chromatography and lastly eluted with THF/PE (1:1) to afford tert-butyl 4-[(5-fluoro-7-{8-fluoro-2-methylimidazo[1,2-a]pyridin-6-yl}cinnolin-4-yl)amino]piperidine-1-carboxylate (73 mg, 78.90%)methyl 6-chloro-2-(6-methoxy-2,7-dimethylindazol-5-yl)quinazoline-4-carboxylate (D25, 456 mg). LCMS: (ES, m/z): 411 [M+H]⁺.

### Synthesis of Intermediate D26

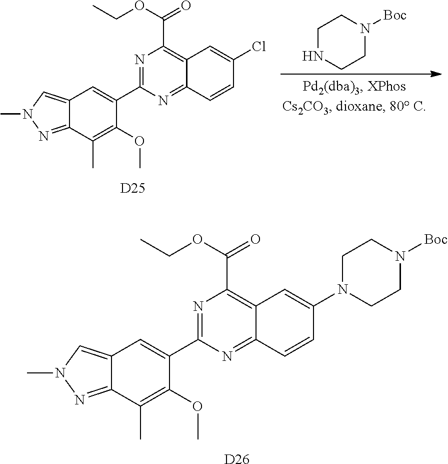

Into a 40 mL vial were added Intermediate D25 (310 mg, 0.781 mmol, 1 equiv), Cs₂CO₃ (765.94 mg, 2.343 mmol, 3 equiv), tert-butyl piperazine-1-carboxylate (174.60 mg, 0.937 mmol, 1.2 equiv), Pd₂(dba)₃ (71.54 mg, 0.078 mmol, 0.1 equiv), XPhos (74.48 mg, 0.156 mmol, 0.2 equiv) and dioxane (6 mL) at room temperature. The resulting mixture was stirred for 3 h at 80° C. under nitrogen atmosphere and later concentrated under reduced pressure. The residue was purified by silica gel column chromatography and eluted with THF/EA (1:1) to afford methyl 6-[4-(tert-butoxycarbonyl) piperazin-1-yl]-2-(6-methoxy-2,7-dimethylindazol-5-yl)quinazoline-4-carboxylate (D26, 240 mg). LCMS: (ES, m/z): 561 [M+H]⁺.

### Synthesis of Intermediate D27

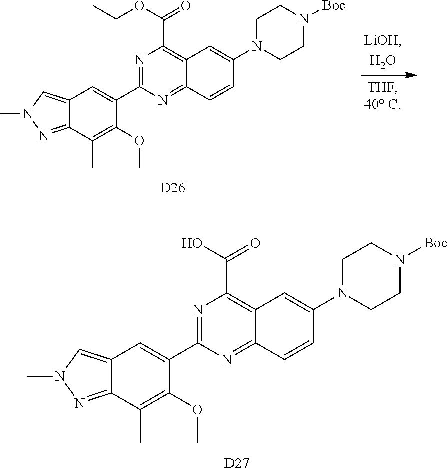

Into a 40 mL vial were added Intermediate D26 (240 mg, 0.439 mmol, 1 equiv), tetrahydrofuran (4 mL), water (2 mL) and lithium (15.77 mg, 0.658 mmol, 1.5 equiv) at room temperature. The resulting mixture was stirred for 3 h at 50° C. and subsequently diluted with water (30 mL). The mixture was acidified to pH 5 with HCl (aq.), and then the aqueous layer was extracted with EtOAc (2×50 mL) and concentrated under reduced pressure, resulting in 6-[4-(tert-butoxycarbonyl) piperazin-1-yl]-2-(6-methoxy-2,7-dimethylindazol-5-yl)quinazoline-4-carboxylic acid (D27, 210 mg). LCMS: (ES, m/z): 533 [M+H]⁺.

### Synthesis of Intermediate D28

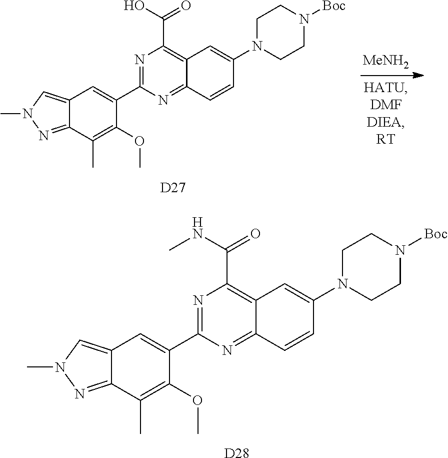

Into a 40 mL vial were added Intermediate D27 (200 mg, 0.376 mmol, 1 equiv), methylamine (34.99 mg, 1.128 mmol, 3 equiv), HATU (214.18 mg, 0.564 mmol, 1.5 equiv), dimethylformamide (6 mL) and DIEA (145.60 mg, 1.128 mmol, 3 equiv) at room temperature. The resulting mixture was stirred for 2 h at room temperature and later quenched by the addition of water (10 mL) at room temperature. The aqueous layer was extracted with EtOAc (2×10 mL) and then dried over Na₂SO₄. The resulting mixture was concentrated under reduced pressure, the residue was purified by silica gel column chromatography, and finally eluted with PE/EA (3:1) to afford tert-butyl 4-[2-(6-methoxy-2,7-dimethylindazol-5-yl)-4-(methylcarbamoyl) quinazolin-6-yl]piperazine-1-carboxylate (D28, 170 mg). LCMS: (ES, m/z): 546 [M+H]⁺.

### Synthesis of Intermediate D29

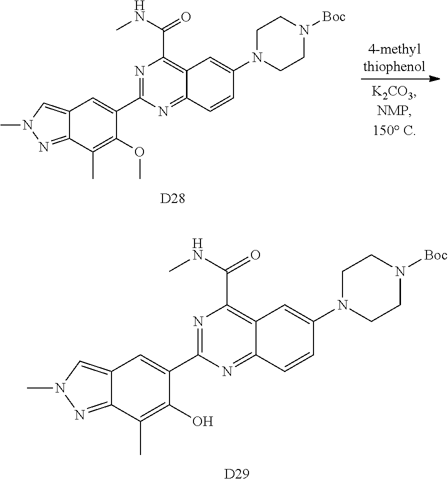

Into an 8 mL round-bottom flask were added Intermediate D28 (80 mg, 0.147 mmol, 1 equiv), NMP (2 mL), K₂CO₃ (20.41 mg, 0.147 mmol, 1 equiv) and 4-methylbenzenethiol (36.42 mg, 0.294 mmol, 2 equiv) at 25° C. The resulting mixture was stirred for 12 h at 150° C. and later allowed to cool to 25° C. The resulting mixture was concentrated under reduced pressure, the residue was purified by silica gel column chromatography, and lastly eluted with THE/EA (1:1) to afford tert-butyl 4-[2-(6-hydroxy-2,7-dimethylindazol-5-yl)-4-(methylcarbamoyl) quinazolin-6-yl]piperazine-1-carboxylate (D29, 50 mg). LCMS: (ES, m/z): 532 [M+H]⁺.

### Synthesis of Compound 501

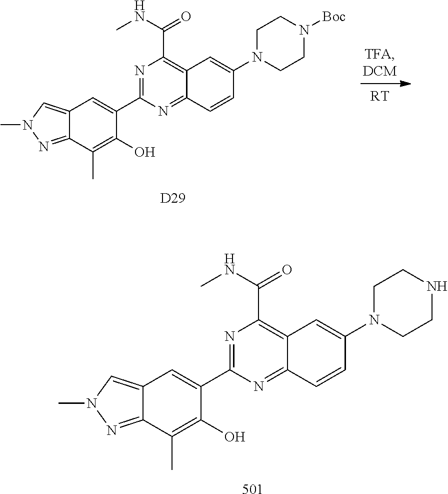 [SMILES: CNC(=O)c1nc(-c2cc3cn(C)nc3c(C)c2O)nc2ccc(N3CCN(C(=O)OC(C)(C)C)CC3)cc12 ; "RT" ; CNC(=O)c1nc(-c2cc3cn(C)nc3c(C)c2O)nc2ccc(N3CCNCC3)cc12 ; "D29" ; "501"]

Into an 8 mL vial were added Intermediate D29 (50 mg, 0.094 mmol, 1 equiv), DCM (1 mL) and trifluoroacetaldehyde (1 mL) at room temperature. The resulting mixture was stirred for 1 h at room temperature. The crude product was then purified by Prep-HPLC following Condition 7, Gradient 3 as previously described to afford 2-(6-hydroxy-2,7-di methylindazol-5-yl)-N-methyl-6-(piperazin-1-yl)quinazoline-4-carboxamide (Compound 501, 6.9 mg). LCMS: (ES, m/z): 432 [M+H]⁺. ¹H NMR: (300 MHz, DMSO-d6) δ 13.33 (s, 1H), 9.24 (d, J=5.0 Hz, 1H), 9.12 (s, 1H), 8.86 (s, 2H), 8.43 (s, 1H), 8.25 (d, J=2.6 Hz, 1H), 8.15-7.97 (m, 2H), 4.17 (s, 3H), 3.56 (d, J=5.6 Hz, 4H), 2.98 (d, J=4.7 Hz, 3H), 2.41 (s, 3H).

### Example 205: Synthesis of Compounds 339 and 340

### Synthesis of Intermediates D32 and D33

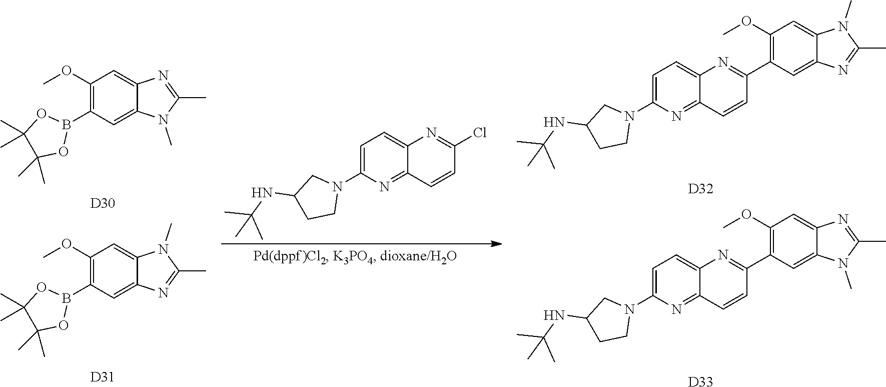

Into a 40 mL round-bottom flask were added the isomers 5-methoxy-1,2-dimethyl-6-(4,4,5,5-tetramethyl-1,3,2-dioxaborolan-2-yl)-1,3-benzodiazole (D30 and D31, 450 mg, 0.596 mmol, 1 equiv, 40%) and N-tert-butyl-1-(6-chloro-1,5-naphthyridin-2-yl)pyrrolidin-3-amine (236 mg, 0.775 mmol, 1.3 equiv), together with dioxane (16 mL) and H₂O (4 mL) at room temperature. To the above mixture was added K₃PO₄ (379 mg, 1.788 mmol, 3 equiv) and Pd(dppf)Cl₂·CH₂Cl₂ (48.52 mg, 0.060 mmol, 0.1 equiv) in portions over 5 min, and the mixture was subsequently stirred for additional 3 h at 90° C. then allowed to cool to room temperature. The resulting mixture was extracted with EA (3×30 mL), the combined organic layers were washed with water (3×30 mL), dried over anhydrous Na₂SO₄, and the filtrate was concentrated under reduced pressure. The residue was purified by reverse flash chromatography following Condition 3, Gradient as previously described to afford the isomers N-tert-butyl-1-[6-(6-methoxy-2,3-dimethyl-1,3-benzodiazol-5-yl)-1,5-naphthyridin-2-yl]pyrrolidin-3-amine (D32 and D33, 45 mg) as white solid. LCMS: (ES, m/z): 445 [M+H].

### Synthesis of Compounds 339 and 340

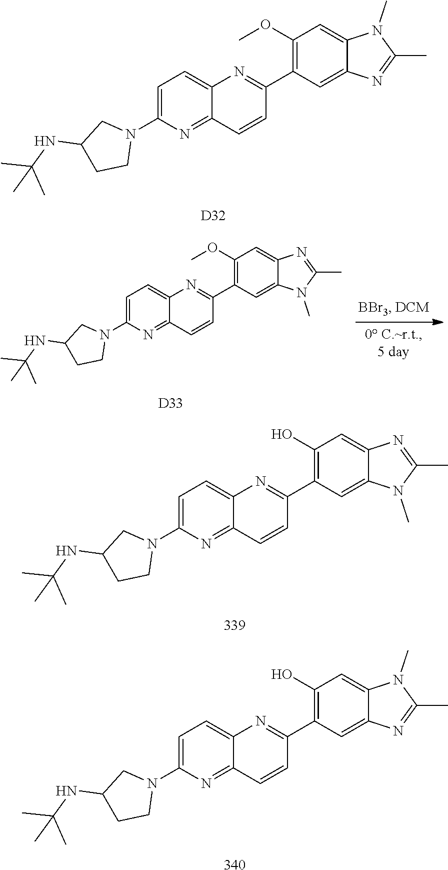

To a stirred solution of Intermediates D32 and D33 (45 mg, 0.101 mmol, 1 equiv) in DCM (2 mL) were added BBr₃ (127 mg, 0.505 mmol, 5 equiv) dropwise at 0° C. The mixture was stirred for 5 days at room temperature, quenched by the addition of MeOH (6 mL) at 0° C. and thereafter concentrated under vacuum. The resulting mixture was extracted with CH₂Cl₂ (3×30 mL), the combined organic layers were washed with water (3×30 mL), dried over anhydrous Na₂SO₄, and the filtrate was concentrated under reduced pressure. The crude product was purified by Prep-HPLC following Condition 2, Gradient 13 as previously described to afford 6-{6-[3-(tert-butylamino)pyrrolidin-1-yl]-1,5-naphthyridin-2-yl}-1,2-dimethyl-1,3-benzodiazol-5-ol (Compound 339, 31.7 mg) and 6-{6-[3-(tert-butylamino) pyrrolidin-1-yl]-1,5-naphthyridin-2-yl}-2,3-dimethyl-1,3-benzodiazol-5-ol (Compound 340, 10.7 mg). Compound 339: LCMS: (ES, m/z): 431 [M+H]. ¹H NMR: (400 MHz, DMSO-d₆) δ 14.10 (s, 1H), 8.50 (d, J=9.3 Hz, 1H), 8.22 (s, 1H), 8.10 (dd, J=9.2, 2.3 Hz, 2H), 7.14 (d, J=9.3 Hz, 1H), 6.95 (s, 1H), 3.97-3.88 (m, 1H), 3.78 (s, 4H), 3.52 (d, J=8.6 Hz, 1H), 3.47 (d, J=9.3 Hz, 1H), 3.29 (d, J=4.5 Hz, 3H), 3.19-3.10 (m, 1H), 2.47-2.18 (m, 1H), 1.92-1.81 (m, 1H), 1.13 (s, 9H). Compound 340: LCMS: (ES, m/z): 431 [M+H]. ¹H NMR: (400 MHz, Chloroform-d) δ 15.01 (s, 1H), 8.19-8.10 (m, 1H), 8.08 (d, J=9.1 Hz, 2H), 7.98 (d, J=9.2 Hz, 1H), 6.91 (d, J=9.2 Hz, 1H), 6.87 (s, 1H), 4.08-3.99 (m, 1H), 3.90-3.82 (m, 1H), 3.68 (s, 3H), 3.66-3.57 (m, 2H), 3.60-3.50 (m, 1H), 2.58 (s, 3H), 2.38-2.26 (m, 1H), 1.92-1.85 (m, 1H), 1.25 (s, 1H), 1.19 (s, 9H).

### Example 206: Synthesis of Compound 344

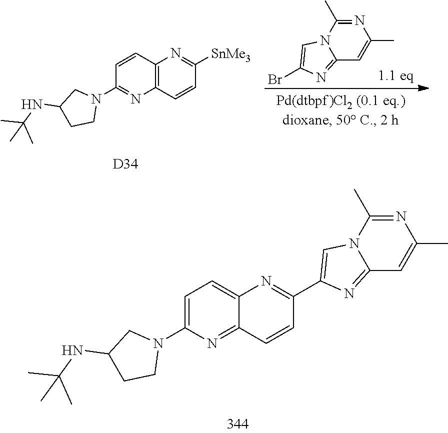

A mixture of 2-bromo-5,7-dimethylimidazo[1,2-c]pyrimidine (D34, 57.41 mg, 0.25 mmol, 1.10 equiv) and Pd(dtbpf)Cl₂ (15.05 mg, 0.02 mmol, 0.10 equiv) in dioxane (2 mL) was stirred for 30 min at 100° C. under nitrogen atmosphere. N-tert-butyl-1-[6-(trimethylstannyl)-1,5-naphthyridin-2-yl]pyrrolidin-3-amine (100 mg, 0.23 mmol, 1.00 equiv) was added and the mixture was stirred for 2 h at 50° C. The residue was purified by silica gel column chromatography and eluted with PE/EA (5:1) to afford crude product, which was purified by Prep-HPLC with the following conditions (2 #SHIMADZU (HPLC-01)): Column, YMC-Actus Triart C18, 30*150 mm, 5 μm; mobile phase, Water (10 mmol/L NH₄HCO₃) and ACN (10% ACN up to 70% in 8 min). This resulted in N-tert-butyl-1-(6-{5,7-dimethylimidazo[1,2-c]pyrimidin-2-yl}-1,5-naphthyridin-2-yl)pyrrolidin-3-amine (10.8 mg, 11.24%) as a yellow solid. LCMS: (ES, m/z):416 [M+H]⁺. ¹H NMR: (400 MHz, DMSO-d₆) δ 8.36 (d, J=0.8 Hz, 1H), 8.27 (d, J=8.7 Hz, 1H), 8.05 (d, J=9.3 Hz, 1H), 8.01-7.94 (m, 1H), 7.34 (s, 1H), 7.12 (d, J=9.3 Hz, 1H), 3.85 (s, 1H), 3.71 (s, 1H), 3.52 (s, 1H), 3.48 (td, J=10.2, 9.4, 6.9 Hz, 1H), 3.12 (dd, J=10.4, 7.2 Hz, 1H), 2.85 (s, 3H), 2.46-2.41 (m, 3H), 2.22-2.12 (m, 1H), 1.76 (dt, J=11.5, 8.5 Hz, 1H), 1.69 (s, 1H), 1.09 (s, 9H).

### Example 207: Synthesis of Compounds 351 and 352

### Synthesis of Intermediate D36

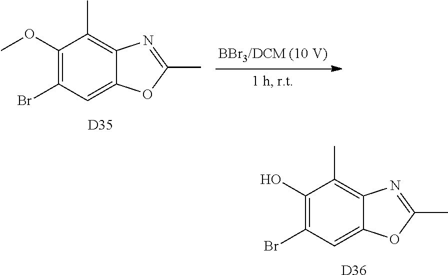

Into a 40 mL vial was added to 6-bromo-5-methoxy-2,4-dimethylbenzo[d]oxazole (D35, 160 mg, 0.624 mmol, 1 equiv) and DCE (1.5 mL), followed by dropwise addition of BBr₃ (2 ml, 3 equiv, 1 M in dioxane) at 0° C. for 10 min. The resulting mixture was warmed to room temperature, stirred for 1 h and then quenched by the addition of 10 mL of MeOH at 0° C., whereupon the solvent was removed in vacuo. The pH of the solution was adjusted to 7 with saturated NaHCO₃, extracted with 3×10 mL of ethyl acetate and the organic layers combined and dried over anhydrous sodium sulfate and concentrated, which resultingly afforded 140 mg of 6-bromo-2,4-dimethylbenzo[d]oxazol-5-ol (D36, crude) which was used directly in the subsequent step.

### Synthesis of Intermediate D37

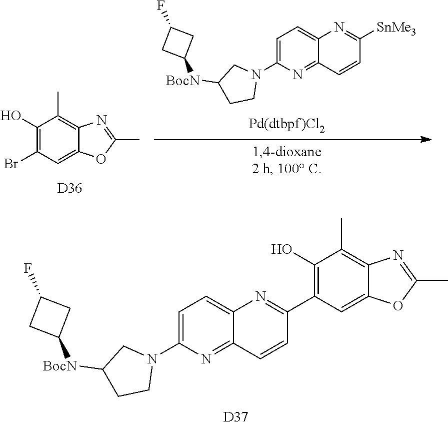

Into a 40 ml vial purged and maintained with an inert atmosphere of nitrogen, was placed Intermediate D36 (140 mg, 0.62 mmol, 1.00 equiv), tert-butyl ((1r,3r)-3-fluorocyclobutyl)(1-(6-(trimethylstannyl)-1,5-naphthyridin-2-yl)pyrrolidin-3-yl)carbamate (340 mg, 0.62 mmol, 1.00 equiv), Pd(dtbpf)Cl₂ (40 mg, 0.062 mmol, 0.1 equiv) and 1,4-dioxane (20 mL). The resulting solution was stirred for 2 h at 100° C., subsequently cooled to room temperature and extracted with 3×20 mL of ethyl acetate. The organic layers were combined and dried over anhydrous sodium sulfate and concentrated. The residue was applied onto a silica gel column with ethyl acetate/hexane (1:1), resulting in 100.0 mg of solid containing Intermediate D37. LCMS: (ES, m/z): 548 [M+H]⁺.

### Synthesis of Intermediate D38

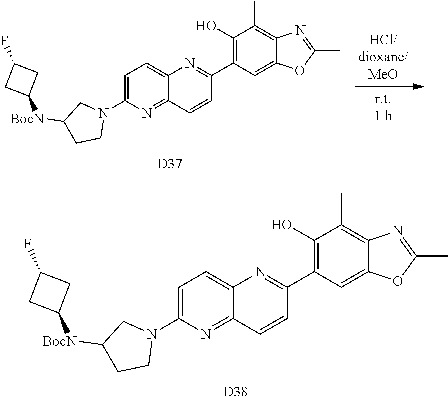 [SMILES: Cc1nc2c(C)c(O)c(-c3ccc4nc(N5CCC(N(C(=O)OC(C)(C)C)[C@H]6C[C@H](F)C6)C5)ccc4n3)cc2o1 ; Cc1nc2c(C)c(O)c(-c3ccc4nc(N5CCC(N(C(=O)OC(C)(C)C)[C@H]6C[C@H](F)C6)C5)ccc4n3)cc2o1 ; "D37" ; "D38"]

Into a 40-mL round-bottom flask was placed Intermediate D37 (100 mg, 0.183 mmol, 1.00 equiv), MeOH (1.00 mL), HCl (g) in dioxane (1.00 ml, 1 mol/L, 5 equiv.). The resulting solution was stirred for 1 h at room temperature, concentrated, and the crude product was purified by prep-HPLC following Condition 3, Gradient 25 as previously described to afford 40 mg of 7-fluoro-5-(6-(3-(((1r,3r)-3-fluorocyclobutyl)amino)pyrrolidin-1-yl)-1,5-naphthyridin-2-yl)-2-methyl-2H-indazol-6-ol (D38). LCMS: (ES, m/z): 448 [M+H]⁺.

### Synthesis of Compounds 351 and 352

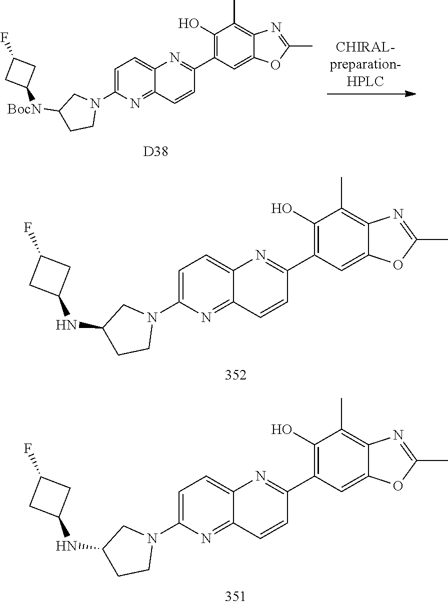 [SMILES: Cc1nc2c(C)c(O)c(-c3ccc4nc(N5CCC(N(C(=O)OC(C)(C)C)[C@H]6C[C@H](F)C6)C5)ccc4n3)cc2o1 ; Cc1nc2c(C)c(O)c(-c3ccc4nc(N5CC[C@@H](N[C@H]6C[C@H](F)C6)C5)ccc4n3)cc2o1 ; "D38" ; Cc1nc2c(C)c(O)c(-c3ccc4nc(N5CC[C@H](N[C@H]6C[C@H](F)C6)C5)ccc4n3)cc2o1]

Intermediate D38 (40 mg) was purified by preparative chiral HPLC following Condition 4, Gradient 7 as previously described to afford 11.3 mg 6-(6-((R)-3-(((1r,3R)-3-fluorocyclobutyl)amino)pyrrolidin-1-yl)-1,5-naphthyridin-2-yl)-2,4-dimethylbenzo[d]oxazol-5-ol (Compound 352) and 11.1 mg 6-(6-((S)-3-(((1r,3S)-3-fluorocyclobutyl)amino)pyrrolidin-1-yl)-1,5-naphthyridin-2-yl)-2,4-dimethylbenzo[d]oxazol-5-ol (Compound 351) LCMS: (ES, m/z): 448 [M+H]⁺. Absolute stereochemistry of the compounds has been assigned arbitrarily. 1H NMR of Compound 352:(400 MHz, DMSO-d6): δ 14.97 (s, 1H), 8.43 (d, J=9.3 Hz, 1H), 8.26 (s, 1H), 8.18 (d, J=9.2 Hz, 1H), 8.09 (d, J=9.1 Hz, 1H), 7.20 (d, J=9.3 Hz, 1H), 5.35-5.09 (m, 1H), 3.82-3.75 (m, 1H), 3.75-3.64 (m, 2H), 3.63-3.51 (m, 1H), 3.46 (d, J=12.1 Hz, OH), 2.61 (s, 3H), 2.47-2.27 (m, 5H), 2.31-2.05 (m, 3), 2.05-1.85 (m, 1H). ¹H NMR of Compound 351: (400 MHz, DMSO-d₆): δ 14.96 (s, 1H), 8.43 (d, J=9.3 Hz, 1H), 8.26 (s, 1H), 8.17 (d, J=9.2 Hz, 1H), 8.09 (d, J=9.1 Hz, 1H), 7.19 (d, J=9.3 Hz, 1H), 5.23 (ddd, J=59.8, 6.4, 3.1 Hz, 1H), 2.61 (s, 3H), 2.46-2.36 (m, 5H), 2.35-2.13 (m, 3H), 1.93 (d, J=15.6 Hz, 1H) The retention time of the first peak is 2.10 min, which has been assigned to Compound 352. The retention time of the second peak is 2.64 min, which has been assigned to Compound 351.

### Example 208: Synthesis of Compounds 356 and 383

### Synthesis of Intermediate D40

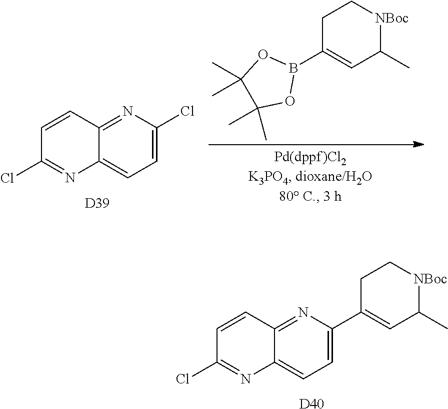

To a stirred solution of 2,6-dichloro-1,5-naphthyridine (D39, 500 mg, 2.512 mmol, 1 equiv) and tert-butyl 2-methyl-4-(4,4,5,5-tetramethyl-1,3,2-dioxaborolan-2-yl)-5,6-dihydro-2H-pyridine-1-carboxylate (1.05 g, 3.266 mmol, 1.3 equiv) in dioxane (16 mL) and H₂O (4 mL) were added Pd(dppf)Cl₂·CH₂Cl₂ (205 mg, 0.251 mmol, 0.1 equiv) and K₂CO₃ (1.04 g, 7.536 mmol, 3 equiv) in portions at room temperature, stirred for additional 3 h at 80° C., and afterward allowed to cool to room temperature. The resulting mixture was extracted with EA (3×30 mL), the combined organic layers were washed with water (3×30 mL), dried over anhydrous Na₂SO₄, and the resulting filtrate was concentrated under reduced pressure. The residue was purified by silica gel column chromatography and eluted with PE/EA (3:1) to afford tert-butyl 4-(6-chloro-1,5-naphthyridin-2-yl)-2-methyl-5,6-dihydro-2H-pyridine-1-carboxylate (D40, 500 mg). LCMS: (ES, m/z): 360 [M+H].

### Synthesis of Intermediate D41

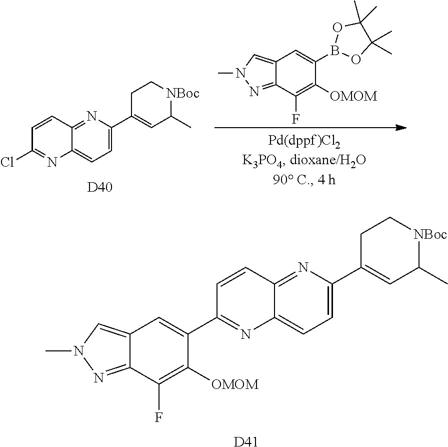

To a stirred solution of Intermediate D40 (450 mg, 1.251 mmol, 1 equiv) and 7-fluoro-6-(methoxymethoxy)-2-methyl-5-(4,4,5,5-tetramethyl-1,3,2-dioxaborolan-2-yl) indazole (630 mg, 1.876 mmol, 1.5 equiv) in 1,4-dioxane (16 mL) and H₂O (4 mL) were added Pd(dppf)Cl₂·CH₂Cl₂ (102 mg, 0.125 mmol, 0.1 equiv) and K₃PO₄ (796 mg, 3.753 mmol, 3 equiv) in portions at room temperature. The resulting mixture was stirred for additional 4 h at 90° C., later allowed to cool to room temperature and extracted with EA (3×30 mL). The combined organic layers were washed with water (3×30 mL), dried over anhydrous Na₂SO₄, and the resulting filtrate was concentrated under reduced pressure. The residue was purified by silica gel column chromatography and eluted with PE/EA (1:1) to afford tert-butyl 4-{6-[7-fluoro-6-(methoxymethoxy)-2-methylindazol-5-yl]-1,5-naphthyridin-2-yl}-2-methyl-5,6-dihydro-2H-pyridine-1-carboxylate (D41, 290 mg). LCMS: (ES, m/z): 534 [M+H].

### Synthesis of Intermediate D42

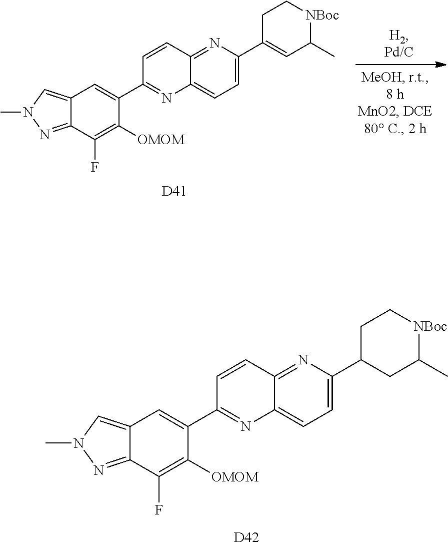

A solution of Intermediate D41 (260 mg, 0.487 mmol, 1.00 equiv) and Pd/C (518 mg, 4.870 mmol, 10 equiv) in MeOH (30 mL) was stirred for 8 h at room temperature under H₂ atmosphere. The resulting mixture was filtered, the filter cake was washed with MeOH (3×10 mL) and concentrated under reduced pressure. The resulting mixture and MnO₂ (424 mg, 4.870 mmol, 10 equiv) in DCE (30 mL) was stirred for 2 h at 80° C., filtered, washed with CH₂Cl₂ (3×10 mL). and concentrated under reduced pressure, affording tert-butyl 4-{6-[7-fluoro-6-(methoxymethoxy)-2-methylindazol-5-yl]-1,5-naphthyridin-2-yl}-2-methylpiperidine-1-carboxylate (D42, 280 mg, 96.56%). LCMS: (ES, m/z): 536 [M+H].

### Synthesis of Intermediate D43

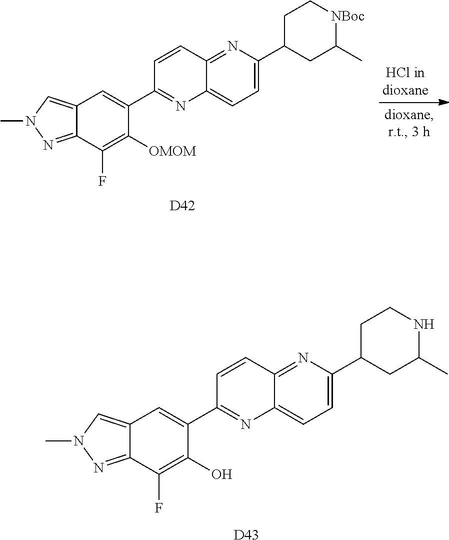

To a stirred solution of Intermediate D42 (250 mg, 0.467 mmol, 1 equiv) in MeOH (10 mL) was added HCl(gas) in 1,4-dioxane (5 mL) dropwise at room temperature. The resulting mixture was stirred for additional 3 h at room temperature and concentrated under vacuum to afford 7-fluoro-2-methyl-5-[6-(2-methylpiperidin-4-yl)-1,5-naphthyridin-2-yl]indazol-6-ol (D43, 150 mg). LCMS: (ES, m/z): 392 [M+H].

### Synthesis of Compounds 356 and 383

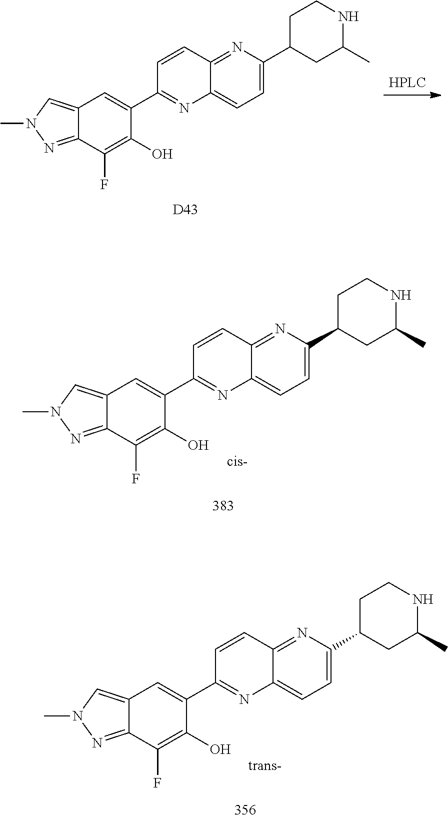

The crude Intermediate D43 (150 mg, 0.766 mmol, 1 equiv)) was purified by preparative chiral HPLC following Condition 4, Gradient 13 as previously described to afford 7-fluoro-2-methyl-5-{6-[(2R,4R)-2-methylpiperidin-4-yl]-1,5-naphthyridin-2-yl}indazol-6-ol (Compound 383, 22.6 mg, 7.53%) and 7-fluoro-2-methyl-5-{6-[(2S,4R)-2-methylpiperidin-4-yl]-1,5-naphthyridin-2-yl}indazol-6-ol (Compound 356, 7.3 mg). Absolute stereochemistry of the compounds was assigned arbitrarily. Compound 383: LCMS: (ES, m/z): 392 [M+H]. ¹H NMR: (400 MHz, DMSO-d₆) δ 14.08 (s, 1H), 8.63 (d, J=9.3 Hz, 1H), 8.57 (dd, J=9.0, 1.9 Hz, 2H), 8.54-8.38 (m, 1H), 8.46 (s, 1H), 7.79 (d, J=8.7 Hz, 1H), 4.18 (s, 3H), 3.17-3.01 (m, 2H), 2.80-2.77 (m, 2H), 1.91 (ddd, J=15.1, 9.2, 6.8 Hz, 2H), 1.69 (qd, J=12.4, 4.1 Hz, 1H), 1.43 (q, J=12.0 Hz, 1H), 1.08 (d, J=6.3 Hz, 3H). ¹⁹F NMR: (376 MHz, DMSO-d₆) δ −157.95. Compound 356: LCMS: (ES, m/z): 392 [M+H]. ¹H NMR: (400 MHz, DMSO-d₆) δ 14.07 (s, 1H), 8.64 (d, J=9.3 Hz, 1H), 8.58 (d, J=1.0 Hz, 1H), 8.56 (d, J=2.7 Hz, 1H), 8.52 (dd, J=9.1, 0.8 Hz, 1H), 8.47 (dd, J=8.6, 0.9 Hz, 1H), 7.84 (d, J=8.8 Hz, 1H), 4.18 (s, 3H), 3.48-3.41 (m, 1H), 3.01-2.90 (m, 1H), 2.96 (s, 2H), 2.18 (ddd, J=13.4, 7.6, 3.9 Hz, 1H), 2.11-2.01 (m, 1H), 1.93 (d, J=11.4 Hz, 1H), 1.73 (dt, J=12.9, 5.8 Hz, 1H), 1.18 (d, J=6.6 Hz, 3H). ¹⁹F NMR: (376 MHz, DMSO-d₆) δ −157.94.

### Example 209: Synthesis of Compound 364

### Synthesis of Intermediate D45

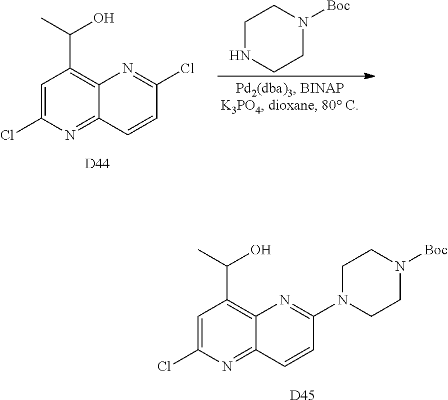

A mixture of 1-(2,6-dichloro-1,5-naphthyridin-4-yl) ethanol (395 mg, 1.625 mmol, 1 equiv)1-(2,6-dichloro-1,5-naphthyridin-4-yl) ethanol (D44, 395 mg, 1.625 mmol, 1 equiv), tert-butyl piperazine-1-carboxylate (363.18 mg, 1.950 mmol, 1.2 equiv), Pd₂(dba)₃ (14.88 mg, 0.016 mmol, 0.01 equiv) and K₃PO₄ (1034.74 mg, 4.875 mmol, 3 equiv) in dioxane (8 mL) was stirred for 5 h at 80° C. under nitrogen atmosphere, concentrated under reduced pressure. The residue was purified by silica gel column chromatography and eluted with PE:EA=4:1 to afford tert-butyl 4-[6-chloro-8-(1-hydroxyethyl)-1,5-naphthyridin-2-yl]piperazine-1-carboxylate (D45, 93 mg). LCMS: (ES, m/z): 393 [M+H]⁺.

### Synthesis of Intermediate D46

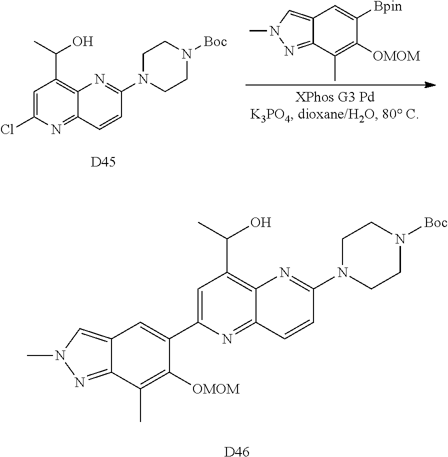

A solution of Intermediate D45 (65 mg, 0.165 mmol, 1 equiv) in dioxane (2 mL) and H₂O (0.2 mL) was treated with 6-(methoxymethoxy)-2,7-dimethyl-5-(4,4,5,5-tetramethyl-1,3,2-dioxaborolan-2-yl)-2H-indazole (65.95 mg, 0.198 mmol, 1.2 equiv), XPhos Pd G3 (14.00 mg, 0.017 mmol, 0.1 equiv) and K₃PO₄ (70.24 mg, 0.330 mmol, 2 equiv) for 3 h at 80° C. under nitrogen atmosphere and later allowed to cool down to room temperature. The resulting mixture was extracted with DCM, the combined organic layers were dried over anhydrous Na₂SO₄, and the filtrate was concentrated under reduced pressure. The residue was purified by silica gel column chromatography and eluted with PE:EA=3:1 to afford tert-butyl 4-[8-(1-hydroxyethyl)-6-[6-(methoxy methoxy)-2,7-dimethylindazol-5-yl]-1,5-naphthyridin-2-yl]piperazine-1-carboxylate (D46, 43 mg). LCMS: (ES, m/z): 563 [M+H]⁺

### Synthesis of Compound 364

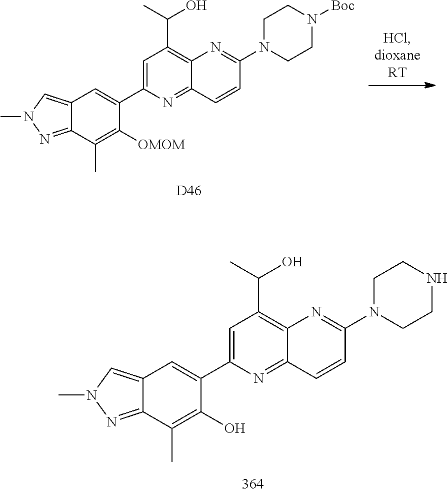

A solution of Intermediate D46 (43 mg, 0.076 mmol, 1 equiv) in HCl (gas) in 1,4-dioxane (2 mL) was stirred for 1 h at room temperature, and subsequently concentrated under reduced pressure. The residue was purified by Prep-TLC following Condition 1, Gradient 1 as previously delineated to afford 5-[4-(1-hydroxyethyl)-6-(piperazin-1-yl)-1,5-naphthyridin-2-yl]-2,7-dimethylindazol-6-ol (Compound 364, 17 mg). LCMS: (ES, m/z): 419 [M+H]⁺. ¹H NMR: (300 MHz, DMSO-d₆) δ 8.49 (s, 1H), 8.39 (d, J=7.4 Hz, 2H), 8.27 (d, J=9.3 Hz, 1H), 7.60 (d, J=9.4 Hz, 1H), 5.65 (q, J=6.4 Hz, 1H), 4.15 (s, 3H), 3.98 (q, J=3.9 Hz, 4H), 3.28 (s, 4H), 2.39 (s, 3H), 1.54 (dd, J=6.7, 2.4 Hz, 3H).

### Example 210: Synthesis of Compound 487

### Synthesis of Intermediate D48

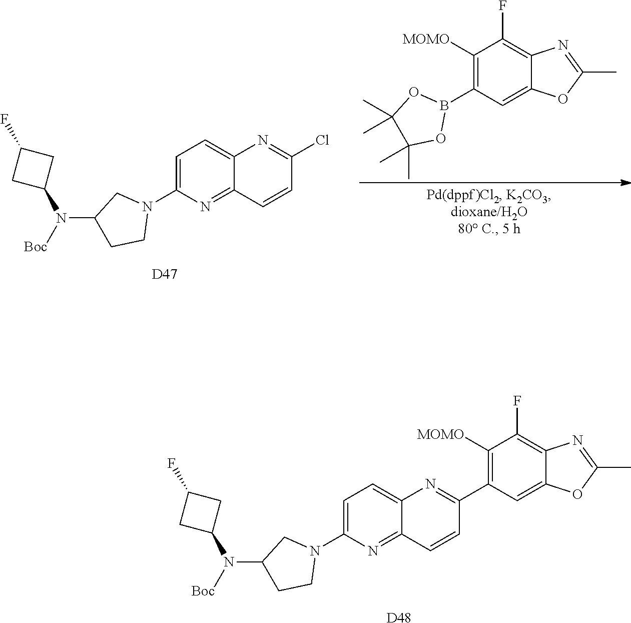

To a solution of tert-butyl N-[1-(6-chloro-1,5-naphthyridin-2-yl)pyrrolidin-3-yl]-N-[(1r,3r)-3-fluorocyclobutyl]carbamate (D47.70 mg, 0.166 mmol, 1 equiv) and 4-fluoro-5-(methoxymethoxy)-2-methyl-6-(4,4,5,5-tetramethyl-1,3,2-dioxaborolan-2-yl)-1,3-benzoxazole (112.14 mg, 0.332 mmol, 2 equiv) in 1,4-dioxane (1 mL) and H₂O (0.25 mL) were added K₂CO₃ (68.95 mg, 0.498 mmol, 3 equiv) and Pd(dppf)Cl₂ (12.17 mg, 0.017 mmol, 0.1 equiv). After stirring for 5 h at 80° C. under a nitrogen atmosphere, the resulting mixture was concentrated under reduced pressure. The residue was purified by Prep-TLC/silica gel column chromatography and eluted with CH₂Cl₂/MeOH (9:1) to afford tert-butyl N-(1-{6-[4-fluoro-5-(methoxymethoxy)-2-methyl-1,3-benzoxazol-6-yl]-1,5-naphthyridin-2-yl}pyrrolidin-3-yl)-N-[(1r,3r)-3-fluorocyclobutyl]carbamate (D48, 30 mg).

### Synthesis of Compound 487

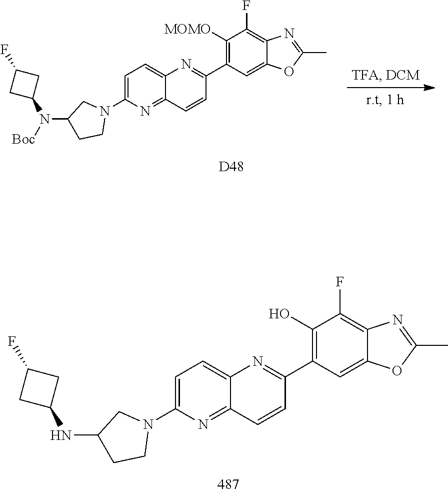

TFA (0.2 mL) was added to a solution of Intermediate D48 (30 mg, 0.050 mmol, 1 equiv) in DCM (1 mL). After stirring for 1 h at room temperature under a nitrogen atmosphere, the resulting mixture was concentrated under reduced pressure. The crude product was purified by Chiral-Prep-HPLC following Condition 12, Gradient 1 as previously described to afford 4-fluoro-2-methyl-6-[6-(3-{[(1r,3r)-3-fluorocyclobutyl]amino}pyrrolidin-1-yl)-1,5-naphthyridin-2-yl]-1,3-benzoxazol-5-ol (Compound 487, 1.6 mg). Absolute stereochemistry of the compound was arbitrarily assigned LCMS: (ES, m/z): 452 [M+H]⁺. ¹H NMR: (400 MHz, DMSO-d₆) δ 15.06 (s, 1H), 8.45 (d, J=9.2 Hz, 1H), 8.33 (d, J=1.4 Hz, 1H), 8.14 (dd, J=22.9, 9.2 Hz, 2H), 7.19 (d, J=9.3 Hz, 1H), 5.19 (dtt, J=56.9, 6.5, 3.6 Hz, 1H), 3.72 (s, 2H), 3.55 (s, 2H), 3.34-3.37 (m, 1H) 2.64 (s, 3H), 2.34-2.43 (m, 3H), 2.14-2.29 (m, 3H), 1.87 (d, J=31.1 Hz, 1H).

### Example 211: Synthesis of Compounds 488 and 489

### Synthesis of Intermediate D50

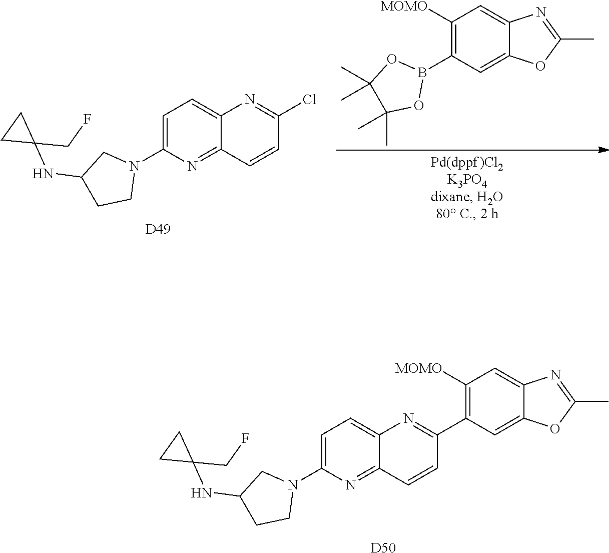

A solution of 1-(6-chloro-1,5-naphthyridin-2-yl)-N-[1-(fluoromethyl)cyclopropyl]pyrrolidin-3-amine (D49, 300 mg, 0.935 mmol, 1 equiv) and K₃PO₄ (595.51 mg, 2.805 mmol, 3 equiv) in dioxane (6 mL) and H₂O (1.2 mL) was treated with Pd(dppf)Cl₂ (68.43 mg, 0.094 mmol, 0.1 equiv) at room temperature under nitrogen atmosphere followed by the addition of 5-(methoxymethoxy)-2-methyl-6-(4,4,5,5-tetramethyl-1,3,2-dioxaborolan-2-yl)-1,3-benzoxazole (358.16 mg, 1.122 mmol, 1.2 equiv) at room temperature. The reaction was stirred for 2 h at 80° C., later allowed to cool to room temperature and concentrated under vacuum. The residue was purified by silica gel column chromatography and eluted with CH₂Cl₂/MeOH (1:1) to afford N-[1-(fluoromethyl)cyclopropyl]-1-{6-[5-(methoxymethoxy)-2-methyl-1,3-benzoxazol-6-yl]-1,5-naphthyridin-2-yl}pyrrolidin-3-amine (D50, 170 mg).

### Synthesis of Compounds 488 and 489

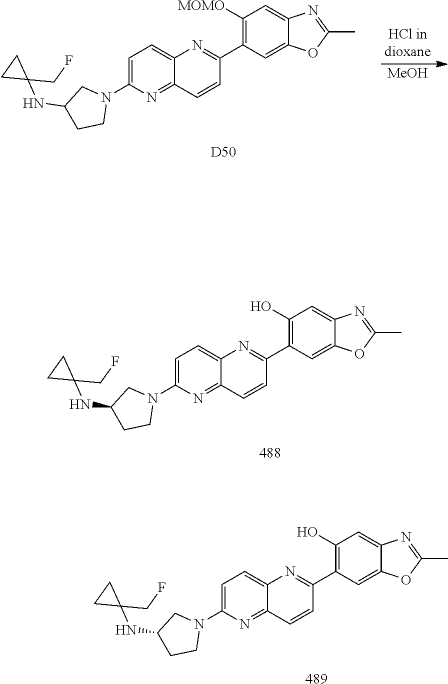

A mixture of Intermediate D50 (170 mg, 0.356 mmol, 1 equiv) and HCl(gas) in 1,4-dioxane (2 mL, 65.826 mmol, 184.91 equiv) in methanol (2 mL, 0.062 mmol, 0.18 equiv) was stirred for 2 h at room temperature under nitrogen atmosphere. The residue product was purified by reverse phase flash chromatography following Condition 5, Gradient 5 to afford 70 mg of solid that was subsequently concentrated under vacuum. The crude product was purified by prep-CHIRAL-HPLC following Condition 13, Gradient 1 to afford 6-{6-[(3R)-3-{[1-(fluoromethyl)cyclopropyl]amino}pyrrolidin-1-yl]-1,5-naphthyridin-2-yl}-2-methyl-1,3-benzoxazol-5-ol (Compound 488, 9.5 mg) and 6-{6-[(3S)-3-{[1-(fluoromethyl)cyclopropyl]amino}pyrrolidin-1-yl]-1,5-naphthyridin-2-yl}-2-methyl-1,3-benzoxazol-5-ol (Compound 489, 9.5 mg). Absolute stereochemistry of the compounds was assigned arbitrarily. The first resolved peak was Compound 488: LCMS: (ES, m/z): 434 [M+H]+. ¹H NMR: (400 MHz, DMSO-d6) δ 14.52 (s, 1H), 8.45-8.39 (m, 2H), 8.10 (dd, J=13.8, 9.2 Hz, 2H), 7.15 (d, J=9.3 Hz, 1H), 7.12 (s, 1H), 4.47 (s, 1H), 4.35 (s, 1H), 3.77 (s, 1H), 3.70 (s, 2H), 3.54 (d, J=8.9 Hz, 1H), 3.32 (d, J=1.4 Hz, 1H), 2.84-2.64 (m, 1H), 2.61 (d, J=1.6 Hz, 3H), 2.14 (s, 1H), 1.91 (s, 1H), 0.62 (s, 4H). The second resolved peak was Compound 489: LCMS: (ES, m/z): 434 [M+H]⁺. ¹H NMR: (400 MHz, DMSO-d6) δ 14.52 (s, 1H), 8.45-8.38 (m, 2H), 8.10 (dd, J=13.5, 9.3 Hz, 2H), 7.15 (d, J=9.3 Hz, 1H), 7.12 (s, 1H), 4.47 (s, 1H), 4.34 (s, 1H), 3.81-3.70 (m, 1H), 3.68 (s, 2H), 3.54 (d, J=9.4 Hz, 1H), 3.32 (d, J=1.4 Hz, 1H), 2.71 (s, 1H), 2.61 (s, 3H), 2.14 (dd, J=12.5, 6.4 Hz, 1H), 1.91 (s, 1H), 0.62 (s, 4H).

### Example 212: Synthesis of Compounds 490, 365, 366, and 367

### Synthesis of Intermediate D52

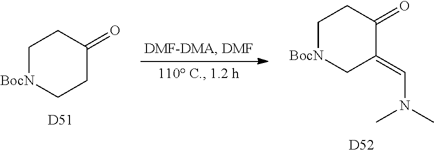

Into a 250 mL round-bottom flask were added tert-butyl 4-oxopiperidine-1-carboxylate (D51, 8 g, 40.151 mmol, 1 equiv), DMF-DMA (1.39 g, 60.227 mmol, 1.5 equiv) and DMF (80 mL). The mixture was stirred for 1.2 h at 110° C., quenched by the addition of water (100 mL) at room temperature, and the aqueous layer was extracted with EtOAc (20 ml×3). The organic layers were washed with brine (20 ml×1), dried over anhydrous Na₂SO₄, and the filtrate was concentrated under reduced pressure to afford tert-butyl (3E)-3-[(dimethylamino)methylidene]-4-oxopiperidine-1-carboxylate (D52, 10 g).

### Synthesis of Intermediate D53

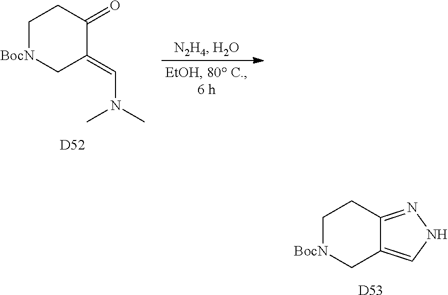

Into a 250 mL sealed tube were added Intermediate D52, (9 g, 35.387 mmol, 1 equiv), hydrazine hydrate (1.97 g, 39.319 mmol, 1 equiv) and EtOH (100 mL). The mixture was stirred for 6 h at 80° C., quenched by the addition of water (100 mL), and concentrated under reduced pressure. The aqueous layer was extracted with EtOAc (3×25 mL), and the combined organic layers were concentrated under reduced pressure. The residue was purified by reverse flash chromatography following Condition 3, Gradient 3 as delineated previously to afford tert-butyl 2H,4H,6H,7H-pyrazolo[4,3-c]pyridine-5-carboxylate (D53, 8 g)

### Synthesis of Intermediates D54 and D55

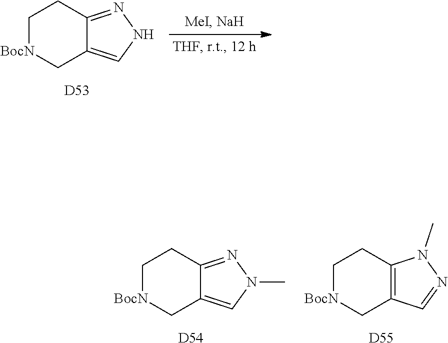

A solution of Intermediate D53 (8 g, 35.830 mmol, 1 equiv) in THE (80 mL) was treated with NaH (1.03 g, 42.996 mmol, 1.2 equiv) for 10 min at 0° C. under nitrogen atmosphere, then Mel (6.10 g, 42.996 mmol, 1.2 equiv) was added at room temperature, and the mixture was stirred for 12 h at room temperature, and later quenched by the addition of water (100 mL) at 0° C. The aqueous layer was extracted with EtOAc (3×50 mL), and the combined organic layers were concentrated under reduced pressure. The crude product was purified by Chiral-Prep-HPLC following Condition 8, Gradient 1 as previously described to afford tert-butyl 2-methyl-4H,6H,7H-pyrazolo[4,3-c]pyridine-5-carboxylate (D54, 1 g) as an oil and tert-butyl 1-methyl-4H,6H,7H-pyrazolo[4,3-c]pyridine-5-carboxylate (D55, 1.2 g) as an oil.

### Synthesis of Intermediate D56

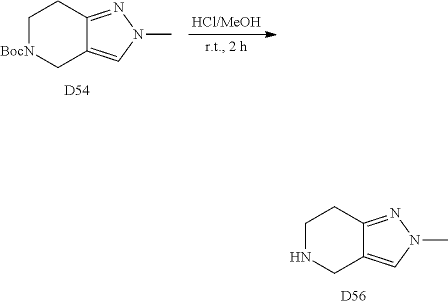

A mixture of Intermediate D54 (1 g, 4.214 mmol, 1 equiv) and HCl(gas) in 1,4-dioxane (10 mL) in MeOH (10 mL) was stirred for 2 h at room temperature under nitrogen atmosphere. The resulting mixture was concentrated under reduced pressure to afford 2-methyl-4H,5H,6H,7H-pyrazolo[4,3-c]pyridine (D56, 450 mg).

### Synthesis of Intermediate D57

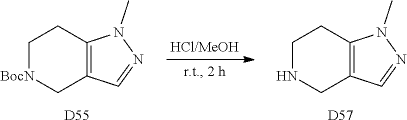

A mixture of Intermediate D55 (1.2 g, 5.057 mmol, 1 equiv) and HCl(gas) in 1,4-dioxane (10 mL) in MeOH (10 mL) was stirred for 2 h at room temperature under nitrogen atmosphere. The resulting mixture was concentrated under reduced pressure to afford 1-methyl-4H,5H,6H,7H-pyrazolo[4,3-c]pyridine (D57, 600 mg).

### Synthesis of Compounds 490 and 366

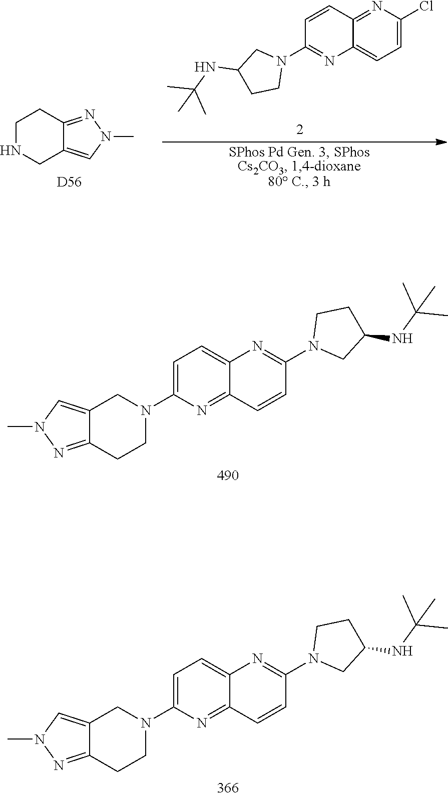

A solution of Intermediate D56 (45.01 mg, 0.328 mmol, 1 equiv) in 1,4-dioxane (3 mL) was treated with N-tert-butyl-1-(6-chloro-1,5-naphthyridin-2-yl)pyrrolidin-3-amine (100 mg, 0.328 mmol, 1.00 equiv), Cs₂CO₃ (213.78 mg, 0.656 mmol, 2 equiv), SPhos Pd Gen.3 (13.47 mg, 0.033 mmol, 0.1 equiv), and SPhos (13.47 mg, 0.033 mmol, 0.1 equiv) for 3 h at 80° C. under nitrogen atmosphere. The resulting mixture was filtered, washed with EtOAc (3×10 mL), and the filtrate was concentrated under reduced pressure. The crude product was purified by Chiral-Prep-HPLC following Condition 14, Gradient 1 to afford the diastereomeric mixture N-(tert-butyl)-1-(6-(2-methyl-2,4,6,7-tetrahydro-5H-pyrazolo[4,3-c]pyridin-5-yl)-1,5-naphthyridin-2-yl)pyrrolidin-3-amine. The product was purified by Chiral-Prep-HPLC with the following following Condition 4, Gradient 8 as previously delineated to afford (3R)-N-tert-butyl-1-(6-{2-methyl-4H,6H,7H-pyrazolo[4,3-c]pyridin-5-yl}-1,5-naphthyridin-2-yl)pyrrolidin-3-amine (Compound 490, 1.9 mg) and (3S)—N-tert-butyl-1-(6-{2-methyl-4H,6H,7H-pyrazolo[4,3-c]pyridin-5-yl}-1,5-naphthyridin-2-yl)pyrrolidin-3-amine (Compound 366, 1.6 mg). Absolute stereochemistry of the compounds was assigned arbitrarily. Compound 490 was resolved from the first peak. LCMS: (ES, m/z): 406[M+H]⁺. ¹H NMR: (400 MHz, DMSO-d₆) δ 7.77-7.68 (m, 2H), 7.49 (s, 1H), 7.31 (d, J=9.3 Hz, 1H), 6.88 (d, J=9.2 Hz, 1H), 4.62 (s, 2H), 3.92 (t, J=5.9 Hz, 2H), 3.75 (s, 3H), 3.59 (s, 2H), 3.48 (s, 2H), 3.02 (s, 2H), 2.71 (t, J=5.8 Hz, 2H), 2.14 (s, 1H), 1.70 (d, J=9.7 Hz, 1H), 1.08 (s, 9H). Compound 366 was resolved from the second peak. LCMS: (ES, m/z): 406[M+H]⁺. ¹H NMR: (400 MHz, DMSO-d₆) δ 7.73 (dd, J=9.1, 6.3 Hz, 2H), 7.49 (s, 1H), 7.31 (d, J=9.4 Hz, 1H), 6.88 (d, J=9.2 Hz, 1H), 4.62 (s, 2H), 3.92 (t, J=5.8 Hz, 2H), 3.75 (s, 3H), 3.59 (t, J=8.2 Hz, 2H), 3.49 (s, 2H), 3.03 (s, 1H), 2.71 (t, J=5.8 Hz, 1H), 2.14 (s, 2H), 1.72 (s, 1H), 1.24 (s, 1H), 1.08 (s, 9H).

### Synthesis of Compounds 365 and 367

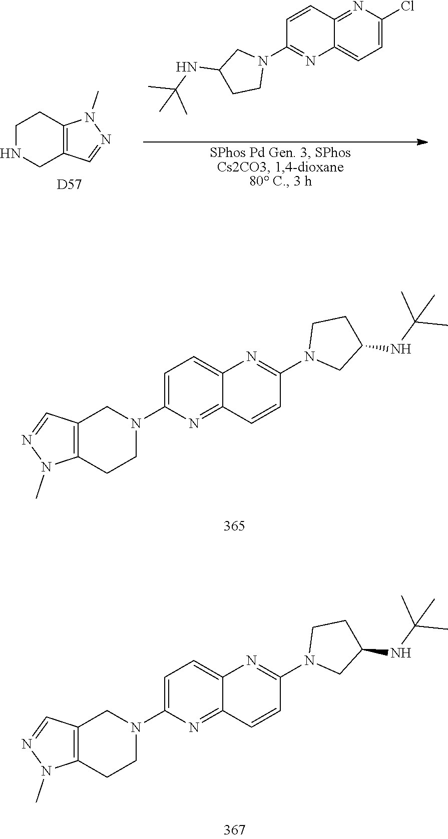

A solution of 1-methyl-4H,5H,6H,7H-pyrazolo[4,3-c]pyridine (D57, 45.01 mg, 0.328 mmol, 1 equiv) in 1,4-dioxane (3 mL) was treated with N-tert-butyl-1-(6-chloro-1,5-naphthyridin-2-yl)pyrrolidin-3-amine (100 mg, 0.328 mmol, 1.00 equiv), Cs₂CO₃ (213.78 mg, 0.656 mmol, 2 equiv), SPhos Pd Gen.3 (13.47 mg, 0.033 mmol, 0.1 equiv), and SPhos (13.47 mg, 0.033 mmol, 0.1 equiv) for 3 h at 80° C. under nitrogen atmosphere. The resulting mixture was filtered, washed with EtOAc (3×10 mL), and the filtrate was concentrated under reduced pressure. The crude product was purified by Chiral-Prep-HPLC following Condition 14, Gradient 1 as previously described. The product was purified by Chiral-Prep-HPLC following Condition 4, Gradient 8 as previously delineated to afford (3S)—N-tert-butyl-1-(6-{1-methyl-4H,6H,7H-pyrazolo[4,3-c]pyridin-5-yl}-1,5-naphthyridin-2-yl)pyrrolidin-3-amine (Compound 365, 1.5 mg) and (3R)-N-tert-butyl-1-(6-{1-methyl-4H,6H,7H-pyrazolo[4,3-c]pyridin-5-yl}-1,5-naphthyridin-2-yl)pyrrolidin-3-amine (Compound 367, 1.6 mg). Absolute stereochemistry was assigned arbitrarily. Compound 365 was resolved from the first peak. LCMS: (ES, m/z): 406[M+H]⁺. ¹H NMR: (400 MHz, DMSO-d₆) δ 7.73 (ddd, J=9.1, 5.0, 0.8 Hz, 2H), 7.33 (d, J=9.3 Hz, 1H), 7.28 (s, 1H), 6.88 (d, J=9.2 Hz, 1H), 4.59 (s, 2H), 3.94 (t, J=5.7 Hz, 2H), 3.76 (dd, J=10.0, 6.9 Hz, 1H), 3.67 (s, 3H), 3.60 (d, J=8.2 Hz, 1H), 3.48 (t, J=7.3 Hz, 1H), 3.41-3.37 (m, 1H), 3.02 (dd, J=10.0, 7.3 Hz, 1H), 2.78 (t, J=5.8 Hz, 2H), 2.13 (d, J=8.3 Hz, 1H), 1.71 (dd, J=11.7, 8.4 Hz, 1H), 1.08 (s, 9H). Compound 367 was resolved from the second peak. LCMS: (ES, m/z): 406[M+H]⁺. ¹H NMR: (400 MHz, DMSO-d₆) δ 7.74 (dd, J=9.2, 3.0 Hz, 2H), 7.34 (d, J=9.2 Hz, 1H), 7.28 (s, 1H), 6.89 (d, J=9.2 Hz, 1H), 4.59 (s, 2H), 3.95 (t, J=5.7 Hz, 2H), 3.77 (s, 1H), 3.67 (s, 3H), 3.60 (s, 1H), 3.48 (t, J=7.3 Hz, 1H), 3.41-3.37 (m, 2H) 3.03 (s, 1H), 2.78 (t, J=5.8 Hz, 2H), 2.13 (s, 1H), 1.73 (s, 1H), 1.10 (s, 9H).

### Example 213: Synthesis of Compound 370

### Synthesis of Intermediate D59

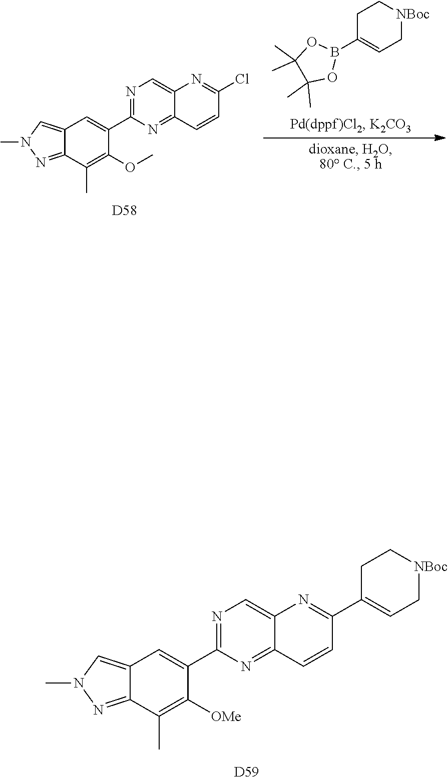

To a stirred solution of 5-{6-chloropyrido[3,2-d]pyrimidin-2-yl}-6-methoxy-2,7-dimethylindazole (D58, 200 mg, 0.589 mmol, 1 equiv) and tert-butyl 4-(4,4,5,5-tetramethyl-1,3,2-dioxaborolan-2-yl)-3,6-dihydro-2H-pyridine-1-carboxylate (200 mg, 0.648 mmol, 1.1 equiv) in dioxane (5 mL) and H₂O (0.5 mL) were added K₂CO₃ (203 mg, 1.472 mmol, 2.5 equiv) and Pd(dppf)Cl₂ (43 mg, 0.059 mmol, 0.1 equiv) in portions at room temperature under nitrogen atmosphere. The resulting mixture was stirred for 5 h at 80° C. under nitrogen atmosphere, and later allowed to cool down to room temperature. The resulting mixture was concentrated under reduced pressure, and the residue was purified by reverse flash chromatography following Condition 10, Gradient 12 as previously delineated to afford tert-butyl 4-[2-(6-methoxy-2,7-dimethylindazol-5-yl)pyrido[3,2-d]pyrimidin-6-yl]-3,6-dihydro-2H-pyridine-1-carboxylate (D59, 87 mg) as a solid. LCMS: (ES, m/z): 487 [M+H]⁺.

### Synthesis of Intermediate D60

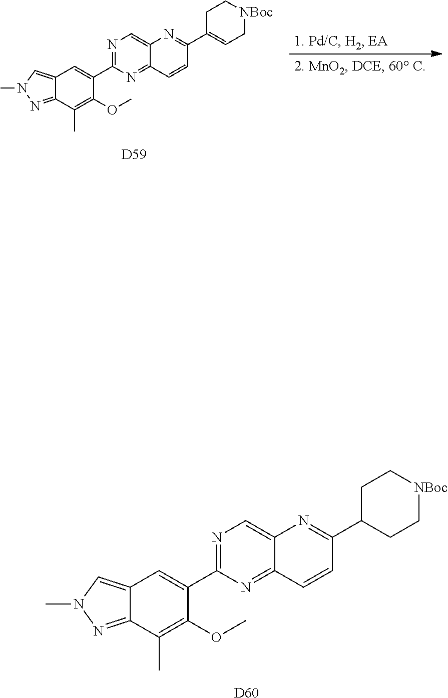 [SMILES: COc1c(-c2ncc3nc(C4=CCN(C(=O)OC(C)(C)C)CC4)ccc3n2)cc2cn(C)nc2c1C ; "1. Pd/C, H2, EA" ; "2. MnO2, DCE, 60° C." ; "D59" ; COc1c(-c2ncc3nc(C4CCN(C(=O)OC(C)(C)C)CC4)ccc3n2)cc2cn(C)nc2c1C ; "D60"]

To a solution of Intermediate D59 (87 mg, 0.179 mmol, 1 equiv) in EA (5 mL) was added Pd/C (38 mg, 0.358 mmol, 2 equiv) under nitrogen atmosphere in a 25 mL round-bottom flask. The mixture was hydrogenated at room temperature for 8 h under hydrogen atmosphere using a hydrogen balloon, filtered through a Celite pad and concentrated under reduced pressure. The residue was dissolved in DCE (5 mL), and MnO₂ (151 mg, 1.730 mmol, 10 equiv) was added in portions at room temperature. The resulting mixture was stirred for 4 h at 60° C. and subsequently allowed to cool to room temperature. The resulting mixture was filtered, washed with CH₂Cl₂ (3×5 mL), and the filtrate was concentrated under reduced pressure to afford tert-butyl 4-[2-(6-methoxy-2,7-dimethylindazol-5-yl)pyrido[3,2-d]pyrimidin-6-yl]piperidine-1-carboxylate (D60, 84 mg). LCMS: (ES, m/z): 489 [M+H]⁺.

### Synthesis of Compound 370

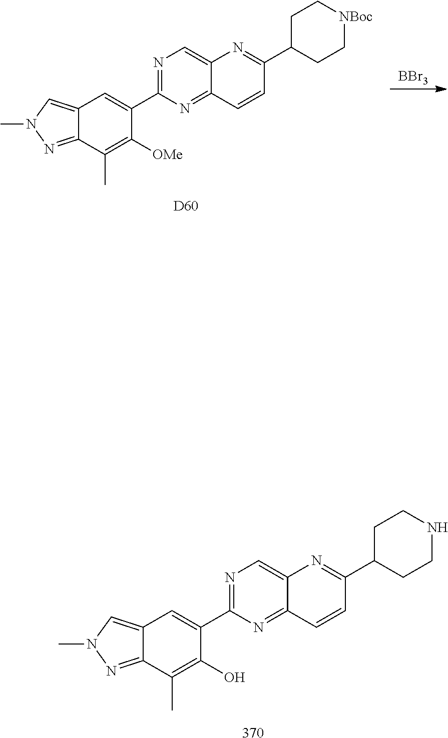

To a stirred solution of Intermediate D60 (82 mg, 0.168 mmol, 1 equiv) in DCE (5 mL) was added boron tribromide (420 mg, 1.680 mmol, 10 equiv) dropwise at room temperature. The resulting mixture was stirred for 0.5 h at 80° C., later allowed to cool to room temperature, quenched by the addition of MeOH (5 mL) at room temperature, and finally concentrated under vacuum. The residue was purified by reverse flash chromatography following Condition 10, Gradient 13 as previously described to afford 2,7-dimethyl-5-[6-(piperidin-4-yl)pyrido[3,2-d]pyrimidin-2-yl]indazol-6-ol (Compound 370, 16 mg) as a solid. LCMS: (ES, m/z): 375 [M+H]+. ¹H NMR: (300 MHz, DMSO-d6) δ 13.23 (s, 1H), 9.71 (d, J=0.8 Hz, 1H), 9.01 (s, 1H), 8.48 (d, J=9.4 Hz, 2H), 8.02 (d, J=8.9 Hz, 1H), 4.16 (s, 3H), 3.11 (s, 1H), 3.05 (d, J=13.0 Hz, 2H), 2.65 (t, J=11.8 Hz, 2H), 2.41 (s, 3H), 1.89 (d, J=12.3 Hz, 2H), 1.83-1.66 (m, 2H).

### Example 214: Synthesis of Compounds 491 and 373

### Synthesis of Intermediates D62 and D63

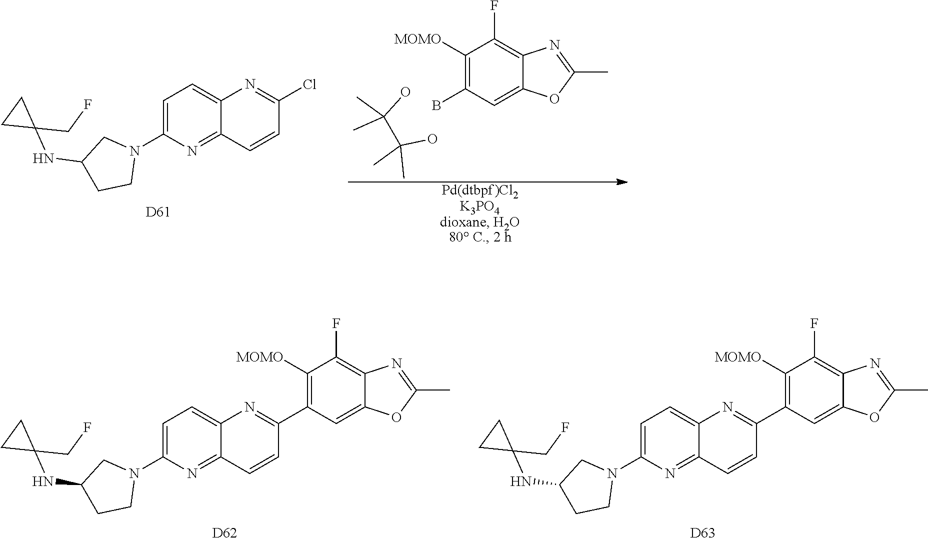

A solution of 1-(6-chloro-1,5-naphthyridin-2-yl)-N-[1-(fluoromethyl)cyclopropyl]pyrrolidin-3-amine (D61, 300 mg, 0.935 mmol, 1 equiv) and 4-fluoro-5-(methoxymethoxy)-2-methyl-6-(4,4,5,5-tetramethyl-1,3,2-dioxaborolan-2-yl)-1,3-benzoxazole (378.35 mg, 1.122 mmol, 1.2 equiv) in dioxane (1.2 mL) and H₂O (6 mL) was treated with Pd(dtbpf)Cl₂ (60.95 mg, 0.094 mmol, 0.1 equiv) at room temperature under nitrogen atmosphere followed by the addition of K₃PO₄ (390.56 mg, 2.805 mmol, 3 equiv) in portions at room temperature. The mixture was stirred for 2 h at 80° C. and later allowed to cool to room temperature. The resulting mixture was concentrated under vacuum, the residue was purified by silica gel column chromatography and eluted with CH₂Cl₂/MeOH (10:1) to afford the crude diastereomeric mixture 1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (200 mg) as a solid. The crude product (200 mg) was purified by Prep-CHIRAL-HPLC following Condition 4, Gradient 14 as previously delineated to afford (3R)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (D62, 60 mg) and (3S)-1-{6-[4-fluoro-5-(methoxymethoxy)-2-methyl-1,3-benzoxazol-6-yl]-1,5-naphthyridin-2-yl}-N-[1-(fluoromethyl)cyclopropyl]pyrrolidin-3-amine (D63, 55 mg). Absolute stereochemistry of the compounds was assigned arbitrarily.

### Synthesis of Compound 491

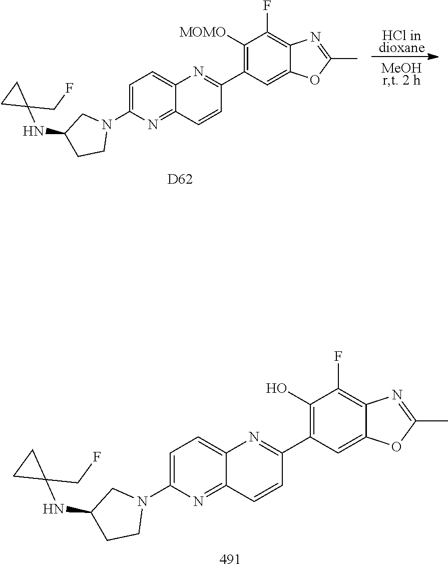

A mixture of Intermediate D62 (60 mg, 0.121 mmol, 1 equiv) and HCl(gas) in 1,4-dioxane (1 mL) in MeOH (1 mL) was stirred for 2 h at room temperature under nitrogen atmosphere and subsequently concentrated under vacuum. The crude product (55 mg) was purified by Prep-HPLC following Condition 1, Gradient 31 as previously described to afford 4-fluoro-6-{6-[(3R)-3-{[1-(fluoromethyl)cyclopropyl]amino}pyrrolidin-1-yl]-1,5-naphthyridin-2-yl}-2-methyl-1,3-benzoxazol-5-ol (Compound 491, 17.6 mg). Absolute stereochemistry of the compound was assigned arbitrarily. LCMS: (ES, m/z): 452 [M+H]⁺. ¹H NMR: (400 MHz, DMSO-d₆) δ 15.06 (s, 1H), 8.44 (d, J=9.2 Hz, 1H), 8.32 (d, J=1.3 Hz, 1H), 8.13 (ddd, J=19.0, 9.2, 0.8 Hz, 2H), 7.17 (d, J=9.3 Hz, 1H), 4.47 (s, 1H), 4.34 (s, 1H), 3.77 (d, J=9.2 Hz, 1H), 3.70 (s, 2H), 3.54 (d, J=9.4 Hz, 1H), 3.34 (s, 1H), 2.71 (d, J=4.1 Hz, 1H), 2.64 (s, 3H), 2.14 (dq, J=12.6, 6.3 Hz, 1H), 1.94-1.86 (m, 1H), 0.62 (d, J=4.6 Hz, 4H).

### Synthesis of Compound 373

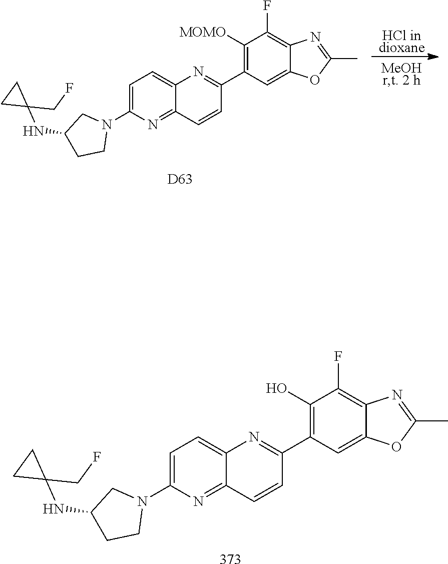

A mixture of Intermediate D63 (55 mg, 0.111 mmol, 1 equiv) and HCl(gas) in 1,4-dioxane (1 mL) in MeOH (1 mL) was stirred for 2 h at room temperature under nitrogen atmosphere then concentrated under vacuum. The crude product (50 mg) was purified by Prep-HPLC following Condition 1, Gradient 32 as previously described to afford 4-fluoro-6-{6-[(3S)-3-{[1-(fluoromethyl)cyclopropyl]amino}pyrrolidin-1-yl]-1,5-naphthyridin-2-yl}-2-methyl-1,3-benzoxazol-5-ol (Compound 373, 9.6 mg). Absolute stereochemistry of the compound was assigned arbitrarily. LCMS: (ES, m/z): 452 [M+H]⁺. ¹H NMR: (400 MHz, DMSO-d₆) δ 15.06 (s, 1H), 8.44 (d, J=9.2 Hz, 1H), 8.32 (d, J=1.4 Hz, 1H), 8.13 (dd, J=19.1, 9.2 Hz, 2H), 7.17 (d, J=9.3 Hz, 1H), 4.47 (s, 1H), 4.34 (s, 1H), 3.78 (s, 1H), 3.70 (s, 2H), 3.54 (d, J=9.3 Hz, 1H), 3.31 (s, 1H), 2.71 (d, J=4.2 Hz, 1H), 2.64 (s, 3H), 2.14 (dt, J=12.4, 6.3 Hz, 1H), 1.90 (s, 1H), 0.62 (d, J=4.5 Hz, 4H).

### Example 215: Synthesis of Compound 400

### Synthesis of Intermediate D65

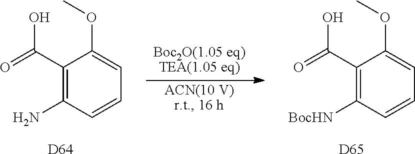

A solution of 2-amino-6-methoxybenzoic acid (D64, 17 g, 101.697 mmol, 1 equiv), Boc₂O (23.30 g, 106.782 mmol, 1.05 equiv) and TEA (10.81 g, 106.782 mmol, 1.05 equiv) in ACN (170 mL) was stirred for 16 h at room temperature. The resulting mixture was poured into water (100 mL) and extracted with CH₂Cl₂ (3×100 mL). The combined organic layers were washed with brine (2×100 mL), dried over anhydrous Na₂SO₄, and the filtrate was concentrated under reduced pressure. The residue was purified by silica gel column chromatography and eluted with CH₂Cl₂/MeOH (1% TEA) (10:1) to afford 2-[(tert-butoxycarbonyl)amino]-6-methoxybenzoic acid (D65, 25 g). LCMS: (ES, m/z):268 [M+H]⁺.

### Synthesis of Intermediate D66

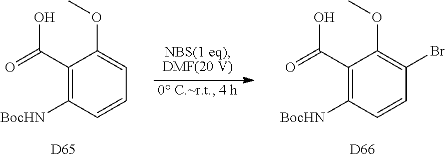

To a stirred solution of Intermediate D65 (21 g, 78.569 mmol, 1 equiv) in ACN (80 mL) was added NBS (13.98 g, 78.569 mmol, 1 equiv) dropwise at 0° C. The resulting mixture was stirred for 4 h at room temperature, subsequently basified to pH 8 with saturated NaHCO₃ (aq.), and later extracted with EtOAc (2×500 mL). The combined organic layers were washed with brine (2×500 mL), dried over anhydrous Na₂SO₄ and the filtrate was concentrated under reduced pressure. The crude product was re-crystallized from DCM/PE (1:5 100 mL) to afford 3-bromo-6-[(tert-butoxycarbonyl)amino]-2-methoxybenzoic acid (D66, 9 g). LCMS: (ES, m/z):346/348 [M+H]⁺.

### Synthesis of Intermediate D67

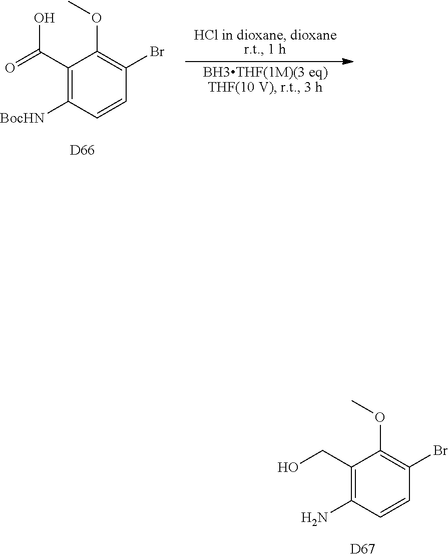

A solution of Intermediate D66 (10 g, 28.887 mmol, 1 equiv) in HCl(gas) in 1,4-dioxane (10 mL) was stirred for 1 h at room temperature and later concentrated under vacuum. The residue was basified to pH 8 with saturated NaHCO₃ (aq.), extracted with CH₂Cl₂ (2×20 mL), dried over anhydrous Na₂SO₄, and the filtrate was concentrated under reduced pressure. BH₃. THE (4.97 g, 57.774 mmol, 2 equiv) was added dropwise to the mixture in THF (10 mL) and allowed to stir overnight at room temperature. The resulting mixture was diluted with MeOH (20 mL) and (1M) HCl and extracted with EtOAc (2×50 mL). The combined organic layers were washed with brine (2×50 mL), dried over anhydrous Na₂SO₄, and the filtrate was concentrated under reduced pressure. The residue was purified by silica gel column chromatography and eluted with PE/EA (1:1) to afford (6-amino-3-bromo-2-methoxyphenyl)methanol (D67, 5 g). LCMS: (ES, m/z):232/234 [M+H]⁺.

### Synthesis of Intermediate D68

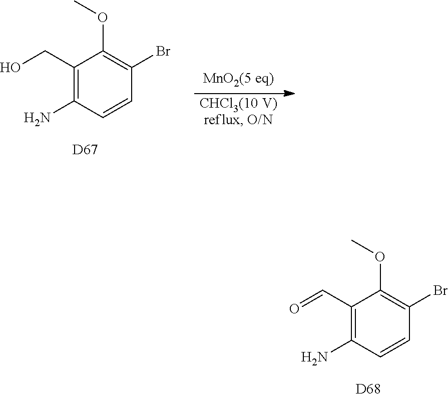

A mixture of Intermediate D67 (5 g, 21.545 mmol, 1 equiv) and MnO₂ (9.36 g, 107.725 mmol, 5 equiv) in CHCl₃ (50 mL) was stirred for 6 h by reflux and later allowed to cool to room temperature. The resulting mixture was filtered, the filter cake was washed with DCM (2×30 mL), and the filtrate was concentrated under reduced pressure. The residue was purified by silica gel column chromatography and eluted with PE/EA (10:1) to afford 6-amino-3-bromo-2-methoxybenzaldehyde (D68, 4 g). LCMS: (ES, m/z):230/232 [M+H]⁺.

### Synthesis of Intermediate D69

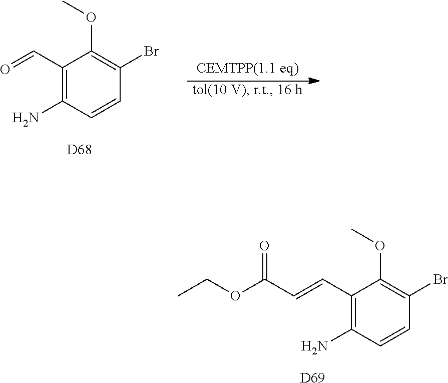

A mixture of Intermediate D68 (4 g, 17.387 mmol, 1 equiv) and ethyl 3-(triphenyl-lambda5-phosphanylidene)propanoate (6.93 g, 19.126 mmol, 1.1 equiv) in toluene (40 mL) was stirred overnight at room temperature and subsequently concentrated under vacuum. The residue was purified by silica gel column chromatography and eluted with PE/EA (5:1) to afford ethyl (2E)-3-(6-amino-3-bromo-2-methoxyphenyl)prop-2-enoate (D69, 4 g). LCMS: (ES, m/z):300/302 [M+H]⁺.

### Synthesis of Intermediate D70

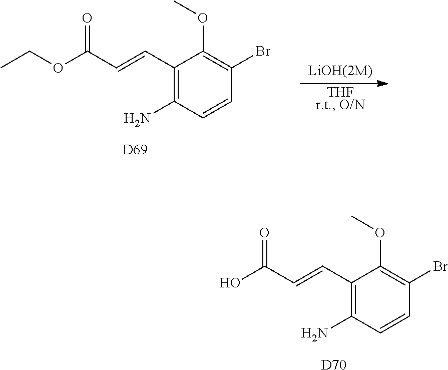

A solution of Intermediate D69 (3.5 g, 11.661 mmol, 1 equiv) and LiOH (2M) (29 mL, 58.305 mmol, 5 equiv) in THE (70 mL) was stirred overnight at room temperature and then acidified to pH 5 with HCl (1M). The resulting mixture was extracted with EtOAc (3×200 mL), dried over anhydrous Na₂SO₄, and the filtrate was concentrated under reduced pressure to afford (2E)-3-(6-amino-3-bromo-2-methoxyphenyl)prop-2-enoic acid (D70, 3 g, 94.55%). LCMS: (ES, m/z):272/274 [M+H]⁺.

### Synthesis of Intermediate D71

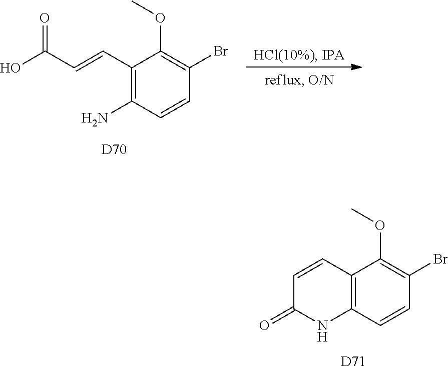

A solution of Intermediate D70 (3 g, 11.025 mmol, 1 equiv) and HCl (15 mL) in IPA (30 mL) was stirred overnight by reflux and later allowed to cool to room temperature. The mixture was then neutralized to pH 7 with saturated NaHCO₃ (aq.), extracted with EtOAc (3×200 mL), dried over anhydrous Na₂SO₄, and the filtrate was concentrated under reduced pressure to afford 6-bromo-5-methoxy-1H-quinolin-2-one (D71, 1.5 g). LCMS: (ES, m/z):254/256 [M+H]⁺.

### Synthesis of Intermediate D72

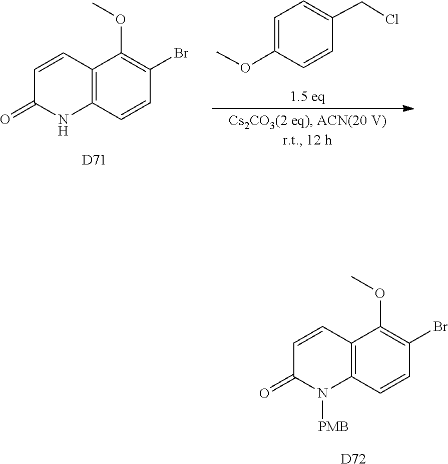

To a stirred mixture of Intermediate D71 (1 g, 3.936 mmol, 1 equiv) and Cs₂CO₃ (2.56 g, 7.872 mmol, 2 equiv) in ACN (20 mL) was added 4-methoxybenzyl chloride (0.92 g, 5.904 mmol, 1.5 equiv) dropwise at 0° C. The resulting mixture was stirred for 12 h at room temperature and then poured into water (100 mL). The resulting mixture was extracted with EtOAc (2×100 mL), dried over anhydrous Na₂SO₄, and the filtrate was concentrated under reduced pressure. The residue was purified by silica gel column chromatography and eluted with PE/EA (5:1) to afford 6-bromo-5-methoxy-1-[(4-methoxyphenyl)methyl]quinolin-2-one (D72, 1 g). LCMS: (ES, m/z):374/376 [M+H]⁺.

### Synthesis of Intermediate D73

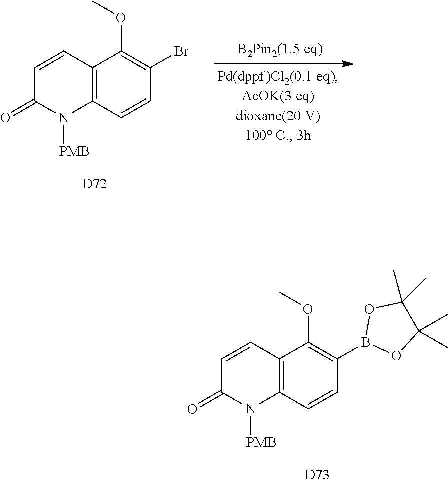

A solution of Intermediate D72 (1 g, 2.672 mmol, 1 equiv), bis(pinacolato)diboron (1.02 g, 4.008 mmol, 1.5 equiv), Pd(dppf)Cl₂·CH₂Cl₂ (0.22 g, 0.267 mmol, 0.1 equiv) and AcOK (0.79 g, 8.050 mmol, 3.01 equiv) in dioxane (20 mL) was stirred for 3 h at 100° C. under nitrogen atmosphere and subsequently allowed to cool to room temperature. The residue was purified by silica gel column chromatography and eluted with PE/EA (1:1) to afford 5-methoxy-1-[(4-methoxyphenyl)methyl]-6-(4,4,5,5-tetramethyl-1,3,2-dioxaborolan-2-yl)quinolin-2-one (D73, 760 mg). LCMS: (ES, m/z):422 [M+H]⁺.

### Synthesis of Intermediate D74

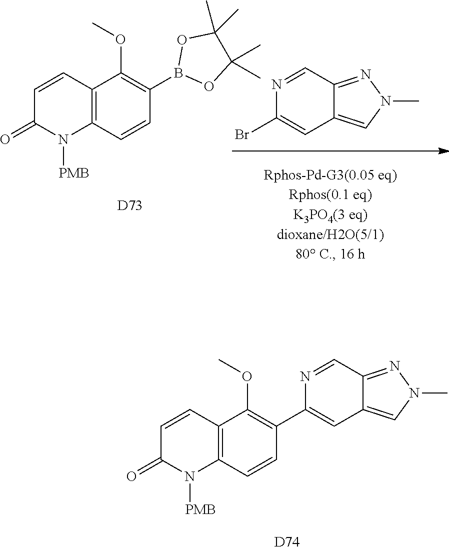

A solution of Intermediate D73 (760 mg, 1.804 mmol, 1 equiv), 5-bromo-2-methylpyrazolo[3,4-c]pyridine (382.53 mg, 1.804 mmol, 1 equiv), RuPhos Palladacycle Gen.3 (42.09 mg, 0.090 mmol, 0.05 equiv), RuPhos (84.18 mg, 0.180 mmol, 0.1 equiv) and K₃PO₄ (1148.74 mg, 5.412 mmol, 3 equiv) in dioxane (5 mL) and H₂O (1 mL) was stirred for 16 h at 80° C. under nitrogen atmosphere. The resulting mixture was extracted with EtOAc (2×50 mL), dried over anhydrous Na₂SO₄, and the filtrate was concentrated under reduced pressure. The residue was purified by silica gel column chromatography and eluted with PE/EA (1:1) to afford 5-methoxy-1-[(4-methoxyphenyl)methyl]-6-{2-methylpyrazolo[3,4-c]pyridin-5-yl}quinolin-2-one (D74, 200 mg). LCMS: (ES, m/z):427 [M+H]⁺.

### Synthesis of Intermediate D75

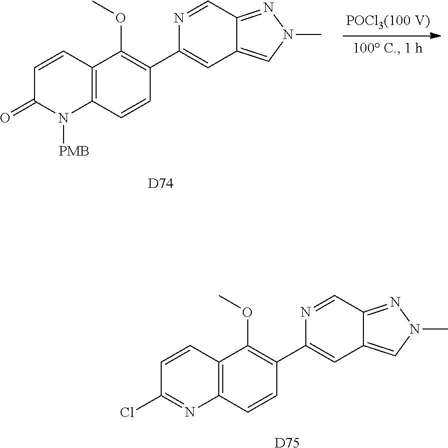

A solution of Intermediate D74 (200 mg, 0.469 mmol, 1 equiv) in POCl₃ (20 mL) was stirred for 1 h at 100° C. and later allowed to cool to room temperature and subsequently concentrated under vacuum. The mixture was basified to pH 8 with saturated NaHCO₃ (aq.), and the residue was purified by silica gel column chromatography and eluted with PE/EA (1:1) to afford 2-chloro-5-methoxy-6-{2-methylpyrazolo[3,4-c]pyridin-5-yl}quinoline (D75, 100 mg). LCMS: (ES, m/z):325 [M+H]⁺.

### Synthesis of Intermediate D76

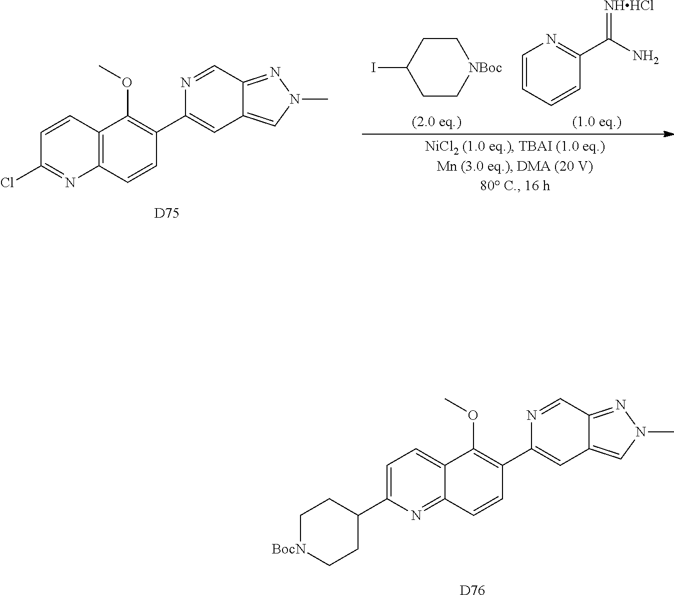

A solution of Intermediate D75 (80 mg, 0.246 mmol, 1 equiv), tert-butyl 4-iodopiperidine-1-carboxylate (153.30 mg, 0.492 mmol, 2 equiv), pyridine-2-carboximidamide hydrochloride (38.82 mg, 0.246 mmol, 1 equiv), NiCl₂ (31.92 mg, 0.246 mmol, 1 equiv). TBAI (90.99 mg, 0.246 mmol, 1 equiv) and Mn (40.60 mg, 0.738 mmol, 3 equiv) in DMA (1.6 mL) was stirred for 16 h at 80° C. under nitrogen atmosphere and subsequently allowed to cool to room temperature. The resulting mixture was extracted with EtOAc (2×50 mL). The combined organic layers were washed with brine (2×50 mL), dried over anhydrous Na₂SO₄, and the filtrate was concentrated under reduced pressure. The residue was purified by silica gel column chromatography and eluted with PE/EA (1:1) to afford tert-butyl 4-(5-methoxy-6-{2-methylpyrazolo[3,4-c]pyridin-5-yl}quinolin-2-yl)piperidine-1-carboxylate (D76, 20 mg). LCMS: (ES, m/z):474 [M+H]⁺.

### Synthesis of Compound 400

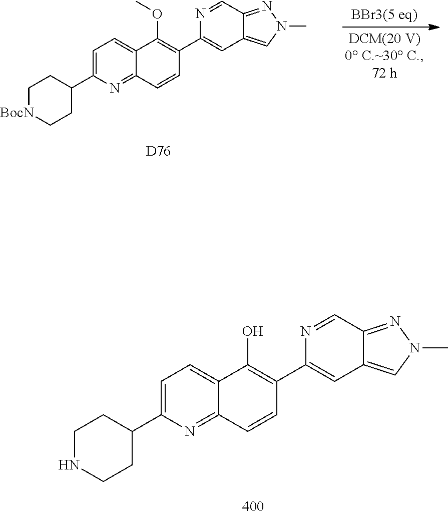 [SMILES: COc1c(-c2cc3cn(C)nc3cn2)ccc2nc(C3CCN(C(=O)OC(C)(C)C)CC3)ccc12 ; Cn1cc2cc(-c3ccc4nc(C5CCNCC5)ccc4c3O)ncc2n1 ; "D76" ; "400"]

To a stirred solution of Intermediate D76 (15 mg, 0.032 mmol, 1 equiv) in DCM (1.5 mL) was added BBr₃ (39.67 mg, 0.160 mmol, 5 equiv) dropwise at 0° C. and allowed to stir for 72 h at room temperature. The resulting mixture was then diluted with MeOH (10 mL) and concentrated under vacuum. The mixture/residue was basified to pH 8 with saturated NaHCO₃ (aq), extracted with CH₂Cl₂ (2×10 mL), dried over anhydrous Na₂SO₄, and the filtrate was concentrated under reduced pressure. The crude product was purified by Chiral-Prep-HPLC following Condition 9, Gradient 2 as previously described to afford 6-{2-methylpyrazolo[3,4-c]pyridin-5-yl}-2-(piperidin-4-yl)quinolin-5-ol (Compound 400, 2.5 mg). LCMS: (ES, m/z):360 [M+H]⁺. ¹H NMR: (400 MHz, DMSO-d₆) δ 15.66 (s, 1H), 9.37 (t, J=1.2 Hz, 1H), 8.68 (s, 1H), 8.62-8.54 (m, 2H), 8.32 (d, J=9.2 Hz, 1H), 7.49-7.40 (m, 2H), 4.34 (s, 3H), 3.09 (d, J=12.0 Hz, 2H), 3.00-2.90 (m, 1H), 2.69-2.64 (m, 2H), 1.85 (d, J=12.6 Hz, 2H), 1.75 (tt, J=12.2, 6.2 Hz, 2H).

### Example 216: Synthesis of Compound 403

### Synthesis of Intermediate D78

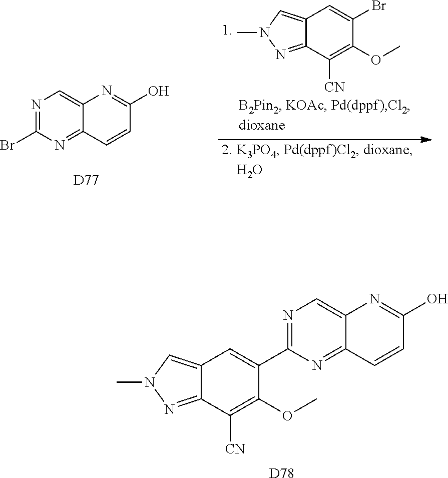

To a stirred solution of 5-bromo-6-methoxy-2-methylindazole-7-carbonitrile (589 mg, 2.212 mmol, 1 equiv) and 4,4,5,5-tetramethyl-2-(tetramethyl-1,3,2-dioxaborolan-2-yl)-1,3,2-dioxaborolane (674 mg, 2.654 mmol, 1.2 equiv) in dioxane (20 mL) were added KOAc (434 mg, 4.424 mmol, 2 equiv) and Pd(dppf)Cl₂ (162 mg, 0.221 mmol, 0.1 equiv) in portions at room temperature under nitrogen atmosphere. The resulting mixture was stirred for 3 h at 90° C. under nitrogen atmosphere and allowed to cool down to room temperature. To the above mixture was added 2-bromopyrido[3,2-d]pyrimidin-6-ol (D77, 500 mg, 2.212 mmol, 1 equiv), Pd(dppf)Cl₂ (162 mg, 0.221 mmol, 0.1 equiv), K₃PO₄ (939 mg, 4.424 mmol, 2 equiv) and H₂O (2 mL) in portions at room temperature and subsequently stirred for an additional 3 h at 90° C., later allowed to cool room temperature, and finally concentrated under reduced pressure. The residue was purified by silica gel column chromatography and eluted with CH₂Cl₂/MeOH (10:1) to afford 5-{6-hydroxypyrido[3,2-d]pyrimidin-2-yl}-6-methoxy-2-methylindazole-7-carbonitrile (D78, 110 mg). LCMS: (ES, m/z): 333 [M+H]⁺.

### Synthesis of Intermediate D79

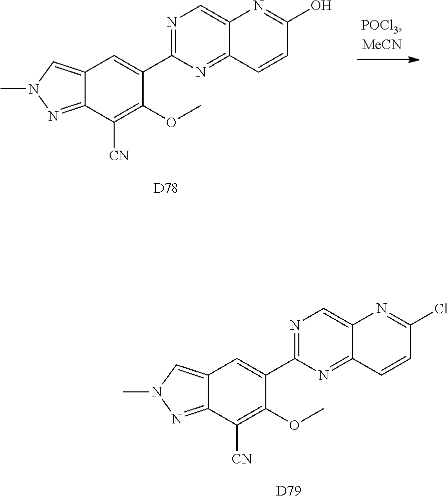

To a stirred solution of Intermediate D78 (110 mg, 0.331 mmol, 1 equiv) in MeCN (5 mL) was added POCl₃ (508 mg, 3.310 mmol, 10 equiv) dropwise at room temperature. The resulting mixture was stirred for 3 h at 80° C., then allowed to cool to room temperature and concentrated under vacuum. The residue was dissolved in EtOAc (20 mL), basified to pH 8 with saturated NaHCO₃ (aq.), and extracted with EtOAc (3×10 mL). The combined organic layers were washed with brine (1×20 mL), dried over anhydrous Na₂SO₄, and the filtrate was concentrated under reduced pressure. The residue was purified by silica gel column chromatography and eluted with PE/EA (1:10) to afford 5-{6-chloropyrido[3,2-d]pyrimidin-2-yl}-6-methoxy-2-methylindazole-7-carbonitrile (D79, 56 mg). LCMS: (ES, m/z): 351 [M+H]⁺.

### Synthesis of Intermediate D80

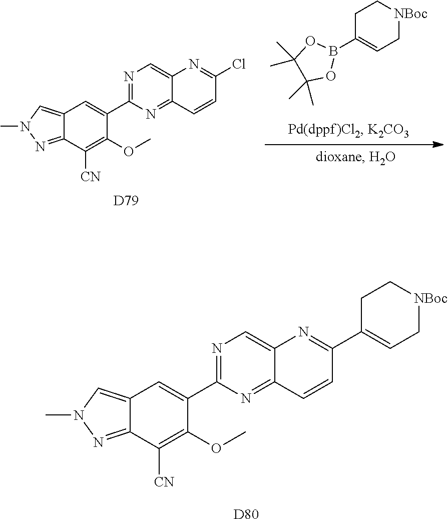

To a stirred solution of Intermediate D79 (55 mg, 0.157 mmol, 1 equiv) and tert-butyl 4-(4,4,5,5-tetramethyl-1,3,2-dioxaborolan-2-yl)-3,6-dihydro-2H-pyridine-1-carboxylate (58 mg, 0.188 mmol, 1.2 equiv) in dioxane (2 mL) and H₂O (0.2 mL) were added K₂CO₃ (54 mg, 0.393 mmol, 2.5 equiv) and Pd(dppf)Cl₂ (12 mg, 0.016 mmol, 0.1 equiv) in portions at room temperature under nitrogen atmosphere, subsequently stirred for 4 h at 90° C. under nitrogen atmosphere, and later allowed to cool to room temperature. The reaction was quenched by the addition of water (20 mL) at room temperature and extracted with EtOAc (3×5 mL). The combined organic layers were washed with brine (3×10 mL), dried over anhydrous Na₂SO₄, and the filtrate was concentrated under reduced pressure. The residue was purified by silica gel column chromatography and eluted with PE/EA (1:10) to afford tert-butyl 4-[2-(7-cyano-6-methoxy-2-methylindazol-5-yl)pyrido[3,2-d]pyrimidin-6-yl]-3,6-dihydro-2H-pyridine-1-carboxylate (D80, 58 mg). LCMS: (ES, m/z): 498 [M+H]⁺.

### Synthesis of Intermediate D81

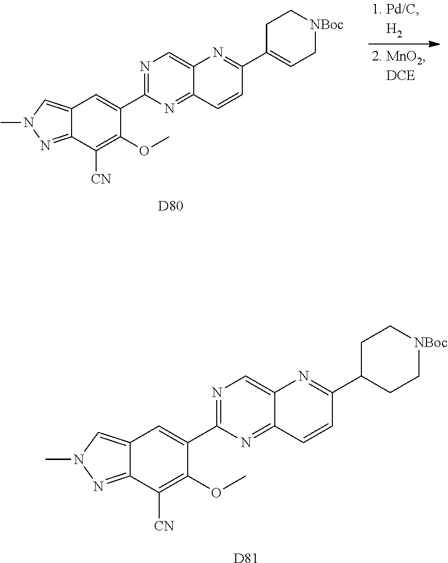 [SMILES: COc1c(-c2ncc3nc(C4=CCN(C(=O)OC(C)(C)C)CC4)ccc3n2)cc2cn(C)nc2c1C#N ; COc1c(-c2ncc3nc(C4CCN(C(=O)OC(C)(C)C)CC4)ccc3n2)cc2cn(C)nc2c1C#N ; "D80" ; "D81"]

To a solution of Intermediate D80 (51 mg, 0.103 mmol, 1 equiv) in MeOH (5 mL) was added Pd/C (55 mg, 0.515 mmol, 5 equiv) under nitrogen atmosphere. The mixture was hydrogenated at room temperature for 5 h under hydrogen atmosphere using a hydrogen balloon, filtered through a Celite pad and concentrated under reduced pressure. The residue was diluted with DCE (5 mL), and MnO₂ (89 mg, 1.030 mmol, 10 equiv) was added in portions at room temperature. The resulting mixture was stirred for an additional 4 h at 60° C. and then allowed to cool to room temperature. The resulting mixture was filtered; the filter cake was washed with DCE (3×10 mL), and the filtrate was concentrated under reduced pressure to afford tert-butyl 4-[2-(7-cyano-6-methoxy-2-methylindazol-5-yl)pyrido[3,2-d]pyrimidin-6-yl]piperidine-1-carboxylate (D81, 50 mg). LCMS: (ES, m/z): 500 [M+H]⁺.

### Synthesis of Compound 403

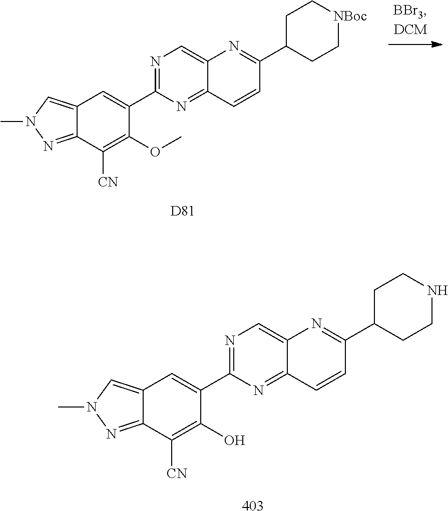 [SMILES: COc1c(-c2ncc3nc(C4CCN(C(=O)OC(C)(C)C)CC4)ccc3n2)cc2cn(C)nc2c1C#N ; Cn1cc2cc(-c3ncc4nc(C5CCNCC5)ccc4n3)c(O)c(C#N)c2n1 ; "D81" ; "403"]

To a stirred solution of Intermediate D81 (60 mg, 0.12 mmol, 1 equiv) in DCE (5 mL) was added BBr₃ (301 mg, 1.2 mmol, 10 equiv) dropwise at room temperature. The resulting mixture was stirred for 0.5 h at 80° C., subsequently allowed to cool to room temperature, and finally concentrated under vacuum. The residue was purified by reverse flash chromatography following Condition 4, Gradient 3 as previously described to afford 6-hydroxy-2-methyl-5-[6-(piperidin-4-yl)pyrido[3,2-d]pyrimidin-2-yl]indazole-7-carbonitrile; trifluoroacetic acid (Compound 403, 13 mg). LCMS: (ES, m/z): 386 [M+H]⁺. ¹H NMR: (300 MHz, Methanol-d₄) δ 9.67 (s, 1H), 9.46 (s, 1H), 8.54-8.42 (m, 2H), 8.02 (d, J=8.8 Hz, 1H), 4.23 (s, 3H), 3.62 (d, J=12.9 Hz, 2H), 3.45 (s, 1H), 3.26 (d, J=12.8 Hz, 2H), 2.43-2.19 (m, 4H).

### Example 217: Synthesis of Compounds 404 and 405

### Synthesis of Intermediate D83

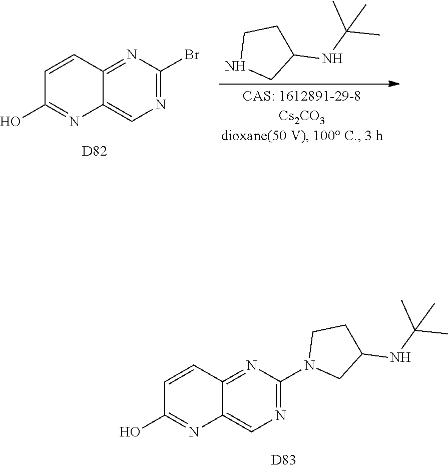

Into a 40 mL vial were added 2-bromopyrido[3,2-d]pyrimidin-6-ol (D82, 250 mg, 1.106 mmol, 1 equiv), dioxane (12.5 mL, 147.550 mmol, 133.40 equiv), N-tert-butylpyrrolidin-3-amine (173.06 mg, 1.217 mmol, 1.1 equiv), Cs₂CO₃ (1081.10 mg, 3.318 mmol, 3 equiv), Pd-PEPPSI-IPentCl 2-methylpyridine (o-picoline (93.04 mg, 0.111 mmol, 0.1 equiv) at room temperature. The resulting mixture was stirred for 3 h at 100° C. under nitrogen atmosphere and subsequently allowed to cool to room temperature. The reaction was quenched by the addition of water (20 mL) at room temperature and extracted with CH₂Cl₂ (3×15 mL). The combined organic layers were washed with brine (1×20 mL), dried over anhydrous Na₂SO₄, and the filtrate was concentrated under reduced pressure. The crude product (D83) was used in the next step directly without further purification. LCMS: (ES, m/z):288 [M+H]⁺.

### Synthesis of Intermediate D84

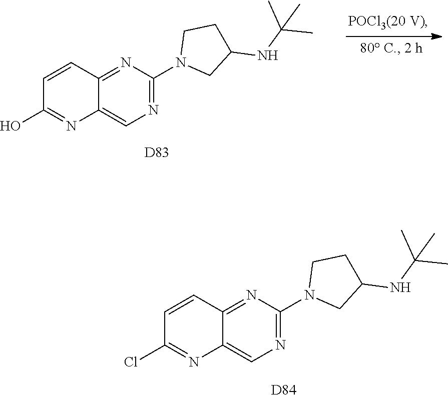

Into a 20 mL vial were added 2-[3-(tert-butylamino)pyrrolidin-1-yl]pyrido[3,2-d]pyrimidin-6-ol (D83,130 mg, 0.452 mmol, 1 equiv) and POCl₃ (2.60 mL, 69.680 mmol, 154.16 equiv) at room temperature, which was stirred for 2 h at 80° C. then allowed to cool to room temperature. The resulting mixture was concentrated under reduced pressure. The crude product (D84) was used in the next step directly without further purification. LCMS: (ES, m/z):306 [M+H]⁺.

### Synthesis of Intermediate D85

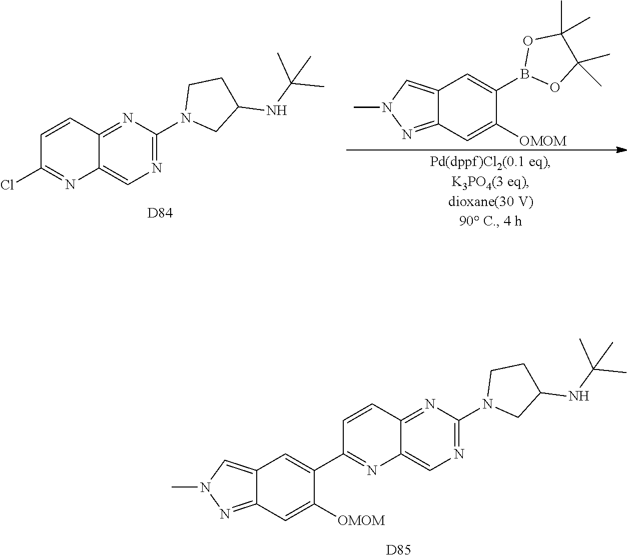

Into a 20 mL vial were added N-tert-butyl-1-{6-chloropyrido[3,2-d]pyrimidin-2-yl}pyrrolidin-3-amine (D84, 180 mg, 0.589 mmol, 1 equiv), 6-(methoxymethoxy)-2-methyl-5-(4,4,5,5-tetramethyl-1,3,2-dioxaborolan-2-yl) indazole (187.28 mg, 0.589 mmol, 1 equiv), dioxane (4 mL, 106.309 mmol, 180.49 equiv), K₃PO₄ (374.82 mg, 1.767 mmol, 3 equiv) in H₂O (0.8 mL) and Pd(dppf)Cl₂·CH₂Cl₂ (47.95 mg, 0.059 mmol, 0.1 equiv) at room temperature. The resulting mixture was stirred for 4 h at 90° C. under nitrogen atmosphere and later allowed to cool to room temperature. The reaction was quenched by the addition of water (20 mL) at room temperature and extracted with EtOAc (3×10 mL). The combined organic layers were washed with brine (1×10 mL), dried over anhydrous Na₂SO₄, and the filtrate was concentrated under reduced pressure. The residue was purified by silica gel column chromatography and eluted with CH₂Cl₂/MeOH (10:1) to afford crude N-tert-butyl-1-{6-[6-(methoxymethoxy)-2-methylindazol-5-yl]pyrido[3,2-d]pyrimidin-2-yl}pyrrolidin-3-amine (D85, 70 mg). LCMS: (ES, m/z):462 [M+H]⁺.

### Synthesis of Compound 404

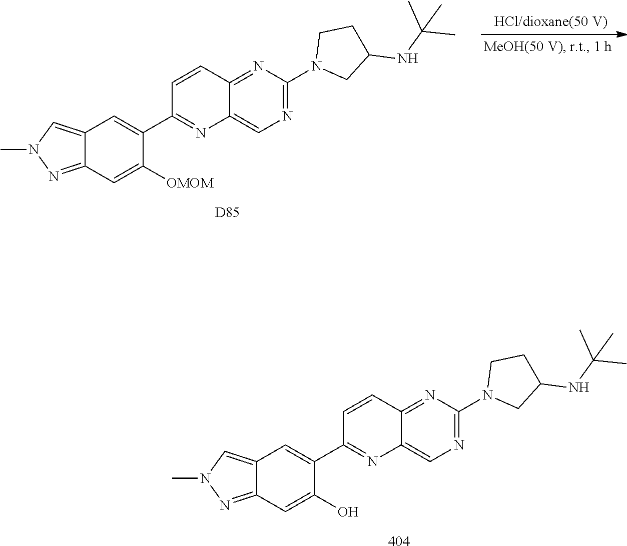

Into a 25 mL vial were added Intermediate D85 (60 mg, 0.130 mmol, 1 equiv) and HCl(gas) in 1,4-dioxane (3 mL) and MeOH (3 mL) at room temperature. The resulting mixture was stirred for 1 h at room temperature and subsequently concentrated under reduced pressure. The crude product (60 mg) was purified by Prep-HPLC following Condition 16, Gradient 1 as previously described to afford 5-{2-[3-(tert-butylamino)pyrrolidin-1-yl]pyrido[3,2-d]pyrimidin-6-yl}-2-methylindazol-6-ol (Compound 404, 4.6 mg). LCMS: (ES, m/z):418 [M+H]⁺. ¹H NMR: (400 MHz, DMSO-d₆) δ 12.68 (s, 1H), 9.23 (d, J=0.8 Hz, 1H), 8.49-8.42 (m, 2H), 8.29 (s, 1H), 8.00 (d, J=9.2 Hz, 1H), 6.90 (s, 1H), 4.11 (s, 3H), 3.94 (dd, J=11.2, 6.8 Hz, 1H), 3.79 (ddd, J=11.9, 8.3, 3.8 Hz, 1H), 3.55 (dq, J=14.8, 7.5 Hz, 2H), 3.20 (dd, J=11.2, 7.1 Hz, 1H), 2.17 (dd, J=10.9, 6.8 Hz, 1H), 1.76 (dq, J=12.3, 8.7 Hz, 1H), 1.54 (s, 1H), 1.10 (s, 9H).

### Synthesis of Compound 405

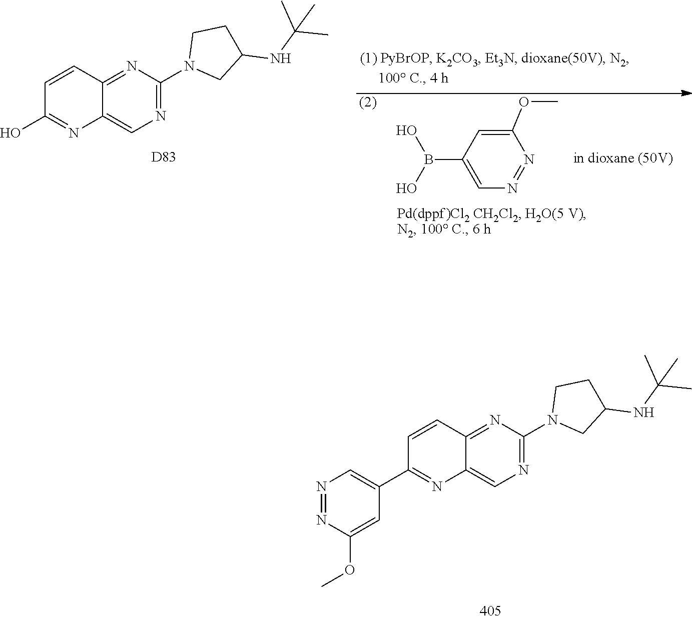

Into a 40 mL vial were added Intermediate D83 (40 mg, 0.139 mmol, 1 equiv), PyBrOP (97.34 mg, 0.209 mmol, 1.5 equiv), K₂CO₃ (57.71 mg, 0.417 mmol, 3 equiv), Et₃N (42.26 mg, 0.417 mmol, 3 equiv) and dioxane (3 mL) at room temperature and stirred for 4 h at 100° C. under nitrogen atmosphere. 6-methoxypyridazin-4-ylboronic acid (42.85 mg, 0.278 mmol, 2 equiv) in dioxane (2 mL), Pd(dppf)Cl₂·CH₂Cl₂ (11.34 mg, 0.014 mmol, 0.1 equiv) and H₂O (0.25 mL, 13.877 mmol, 99.70 equiv) was added to the reaction system at 100° C. under nitrogen atmosphere. The resulting mixture was stirred for 6 h at 100° C. under nitrogen atmosphere and subsequently allowed to cool down to room temperature. The reaction was quenched by the addition of water (5 mL) at room temperature and extracted with CH₂Cl₂ (3×5 mL). The combined organic layers were washed with brine (1×5 mL), dried over anhydrous Na₂SO₄, and the filtrate was concentrated under reduced pressure. The residue was purified by Prep-TLC (CH₂Cl₂/MeOH 10:1) to afford the crude product. The crude product (30 mg) was purified by Prep-HPLC following Condition 16, Gradient 1 as previously delineated to afford N-tert-butyl-1-[6-(6-methoxypyridazin-4-yl)pyrido[3,2-d]pyrimidin-2-yl]pyrrolidin-3-amine (Compound 405, 8.7 mg). LCMS: (ES, m/z):380 [M+H]⁺. ¹H NMR: (400 MHz, DMSO-d₆) δ 9.68 (d, J=1.8 Hz, 1H), 9.34 (s, 1H), 8.49 (d, J=9.0 Hz, 1H), 8.06 (d, J=8.9 Hz, 1H), 7.86 (d, J=1.8 Hz, 1H), 4.11 (s, 3H), 3.94 (dt, J=27.9, 10.0 Hz, 1H), 3.78 (d, J=28.1 Hz, 1H), 3.54 (s, 2H), 3.20 (s, 1H), 2.16 (s, 1H), 1.83-1.71 (m, 2H), 1.08 (s, 9H).

### Example 218: Synthesis of Compound 449

### Synthesis of Intermediate D87

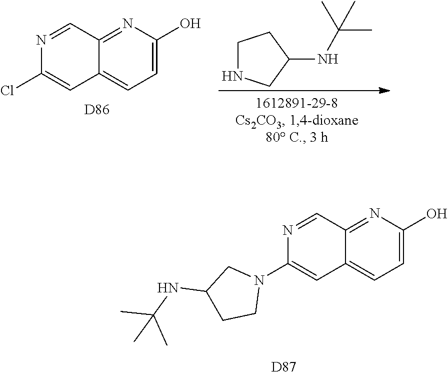

A solution of 6-chloro-1,7-naphthyridin-2-ol (D86, 500 mg, 2.769 mmol, 1 equiv) in 1,4-dioxane (10 mL) was treated with N-tert-butylpyrrolidin-3-amine (393.84 mg, 2.769 mmol, 1 equiv) and Pd-PEPPSI-IPentCl 2-methylpyridine (o-picoline (232.89 mg, 0.277 mmol, 0.1 equiv), and Cs₂CO₃ (2706.29 mg, 8.307 mmol, 3 equiv) for 3 h at 80° C. under nitrogen atmosphere. The reaction was quenched by the addition of water (10 mL) and then extracted with EtOAc (3×5 mL). The combined organic layers were dried over anhydrous Na₂SO₄, and the filtrate was concentrated under reduced pressure. The residue was purified by silica gel column chromatography and eluted with CHCl₃/MeOH (9:1) to afford 6-[3-(tert-butylamino)pyrrolidin-1-yl]-1,7-naphthyridin-2-ol (D87, 400 mg, 50.45%).

### Synthesis of Intermediate D88

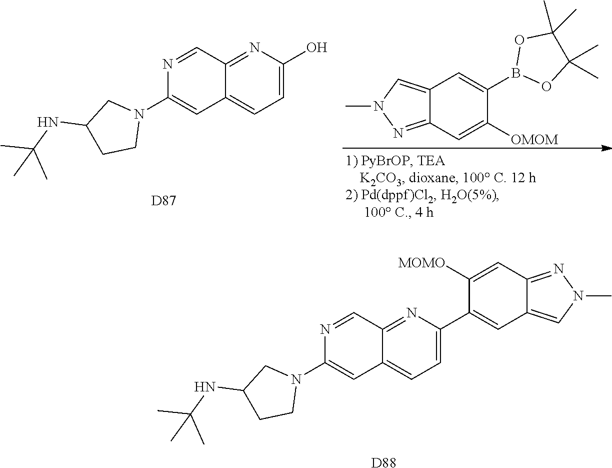

A solution of Intermediate D87 (400 mg, 1.397 mmol, 1 equiv) in 1,4-dioxane (10 mL) was treated with PyBrOP (976.72 mg, 2.095 mmol, 1.5 equiv), and triethylamine (424.01 mg, 4.191 mmol, 3 equiv) for 12 h at 100° C. under nitrogen atmosphere. Then a solution of 6-(methoxymethoxy)-2-methyl-5-(4,4,5,5-tetramethyl-1,3,2-dioxaborolan-2-yl) indazole (444.42 mg, 1.397 mmol, 1 equiv) and Pd(dppf)Cl₂ (113.78 mg, 0.140 mmol, 0.1 equiv) in dioxane (4 mL) and H₂O (100 uL) were added to the reaction system. The resulting mixture was stirred for 4 at 100° C. under nitrogen atmosphere, then quenched by the addition of water (20 mL) at room temperature and extracted with EtOAc (3×15 mL). The combined organic layers were washed with brine (1×20 mL), dried over anhydrous Na₂SO₄, and the filtrate was concentrated under reduced pressure. The residue was purified by silica gel column chromatography and eluted with DCM/MeOH (15:1) to afford N-tert-butyl-1-{2-[6-(methoxymethoxy)-2-methylindazol-5-yl]-1,7-naphthyridin-6-yl}pyrrolidin-3-amine (D88, 130 mg).

### Synthesis of Compound 449

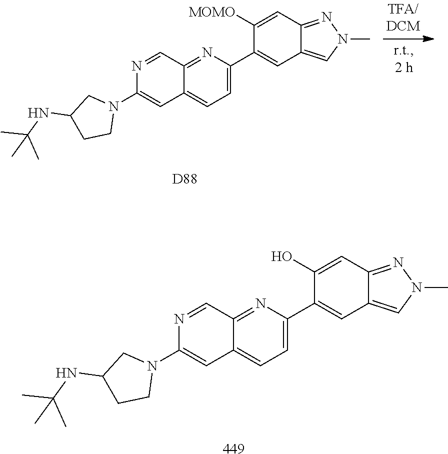

A solution of N-tert-butyl-1-{2-[6-(methoxymethoxy)-2-methylindazol-5-yl]-1,7-naphthyridin-6-yl}pyrrolidin-3-amine (110 mg, 0.239 mmol, 1 equiv) in TFA (1 mL)/DCM (1 mL) was treated for 2 h at room temperature under nitrogen atmosphere. The resulting mixture was concentrated under reduced pressure. The crude product was purified by Chiral-Prep-HPLC following Condition 15, Gradient 1 as previously described to afford 5-{6-[3-(tert-butylamino)pyrrolidin-1-yl]-1,7-naphthyridin-2-yl}-2-methylindazol-6-ol (Compound 449, 18.4 mg). LCMS: (ES, m/z): 417[M+H]⁺. ¹H NMR: (400 MHz, DMSO-d₆) δ 13.74 (s, 1H), 9.02 (d, J=0.9 Hz, 1H), 8.55 (s, 1H), 8.37 (s, 1H), 8.33 (d, 1H), 8.21 (dd, J=9.2, 0.8 Hz, 1H), 6.88 (s, 1H), 6.62 (s, 1H), 4.12 (s, 3H), 3.77 (dd, J=10.1, 6.8 Hz, 1H), 3.60 (m, 1H), 3.54 (m, 1H), 3.46-3.35 (m, 1H), 3.05 (dd, J=10.1, 7.1 Hz, 1H), 2.25-2.14 (m, 1H), 1.77 (dq, J=11.9, 8.6 Hz, 2H), 1.10 (s, 9H).

### Example 219: Synthesis of Compound 458

### Synthesis of Intermediate D90

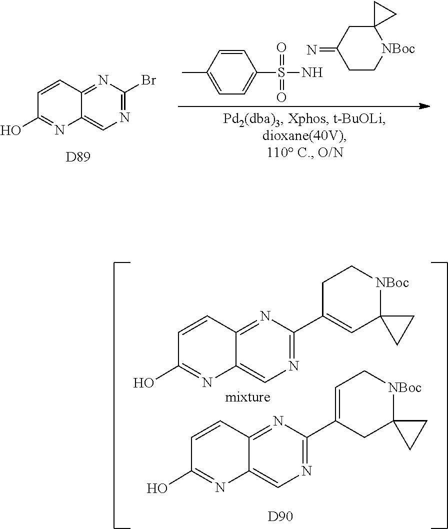

Into a 40 mL vial were added 2-bromopyrido[3,2-d]pyrimidin-6-ol (D89, 500 mg, 2.212 mmol, 1 equiv), tert-butyl (7E)-7-[(4-methylbenzenesulfonamido)imino]-4-azaspiro[2.5]octane-4-carboxylate (870.45 mg, 2.212 mmol, 1 equiv), t-BuOLi (531.29 mg, 6.636 mmol, 3 equiv), dioxane (20 mL), Pd₂(dba)₃ (202.57 mg, 0.221 mmol, 0.1 equiv) and Xphos (105.46 mg, 0.221 mmol, 0.1 equiv) at room temperature. The resulting mixture was stirred overnight at 110° C. under nitrogen atmosphere and subsequently allowed to cool to room temperature. The reaction was quenched by the addition of water (30 mL) at room temperature and extracted with CH₂Cl₂/MeOH (3:1) (4×30 mL). The combined organic layers were washed with brine (1×20 mL), dried over anhydrous Na₂SO₄, and the filtrate was concentrated under reduced pressure. The residue was purified by trituration with ethyl acetate:PE=5:1 (20 mL), and the precipitated solids were collected by filtration and washed with PE (3×3 mL). The crude product (D90) was used in the next step directly without further purification. LCMS: (ES, m/z):355 [M+H]⁺.

### Synthesis of Intermediate D91

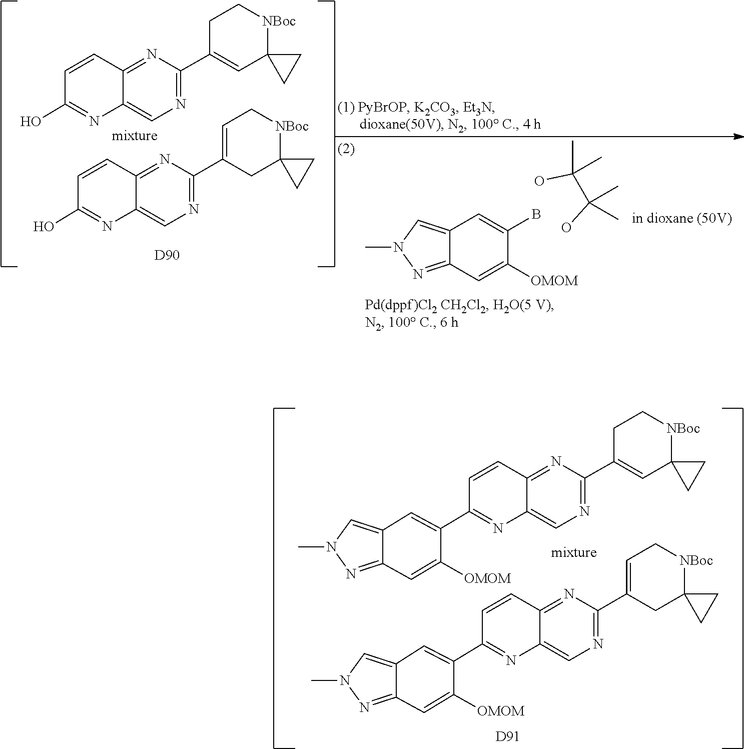

Into a 40 mL vial were added tert-butyl 7-{6-hydroxypyrido[3,2-d]pyrimidin-2-yl}-4-azaspiro[2.5]oct-7-ene-4-carboxylate (D90, 270 mg, 0.762 mmol, 1 equiv), K₂CO₃ (315.87 mg, 2.286 mmol, 3 equiv), Et₃N (231.28 mg, 2.286 mmol, 3 equiv) and dioxane (13.5 mL) at room temperature, and the resulting mixture was stirred for 4 h at 100° C. under nitrogen atmosphere. A solution of 6-(methoxymethoxy)-2-methyl-5-(4,4,5,5-tetramethyl-1,3,2-dioxaborolan-2-yl) indazole (484.80 mg, 1.524 mmol, 2 equiv) in 1,4-dioxane was added, followed by addition of Pd(dppf)Cl₂·CH₂Cl₂ (62.06 mg, 0.076 mmol, 0.1 equiv) and H₂O (0.61 mL) The resulting mixture was stirred for 6 h at 100° C. under nitrogen atmosphere and later allowed to cool to room temperature. The reaction was quenched by the addition of water (20 mL) at room temperature and then extracted with EtOAc (3×15 mL). The combined organic layers were washed with brine (1×20 mL), dried over anhydrous Na₂SO₄, and the filtrate was concentrated under reduced pressure. The residue was purified by silica gel column chromatography and eluted with PE/EA (1:4) to afford the mixture (D91, 220 mg) as a solid. LCMS: (ES, m/z):529 [M+H]⁺.

### Synthesis of Intermediate D92

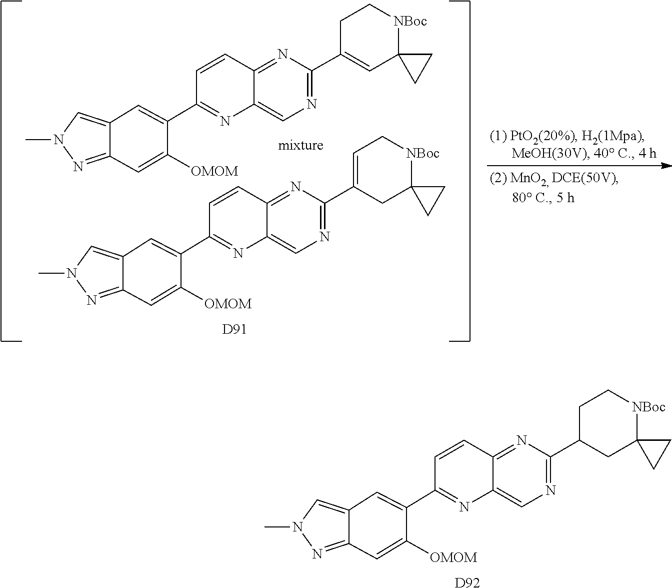

Into a 25 mL round-bottom flask were added tert-butyl 7-{6-[6-(methoxymethoxy)-2-methylindazol-5-yl]pyrido[3,2-d]pyrimidin-2-yl}-4-azaspiro[2.5]oct-7-ene-4-carboxylate (D91 isomer, 80 mg, 0.151 mmol, 1 equiv), MeOH (2.4 mL, 74.096 mmol, 489.60 equiv) and PtO₂ (6.87 mg, 0.030 mmol, 0.2 equiv) at room temperature. The resulting mixture was stirred for 4 h at 40° C. under hydrogen (1 MPa) atmosphere. The resulting mixture was filtered, washed with MeOH (3×3 mL), and concentrated under reduced pressure. Into an 8 mL vial were added the mixture, DCE (3 mL, 37.898 mmol, 250.42 equiv) and MnO₂ (263.14 mg, 3.020 mmol, 20 equiv) at room temperature. The resulting mixture was stirred for 5 h at 80° C. and subsequently filtered, washed with MeOH (3×3 mL), and concentrated under reduced pressure. The crude product (D92) was used in the next step directly without further purification. LCMS: (ES, m/z): 531[M+H]⁺.

### Synthesis of Compound 448

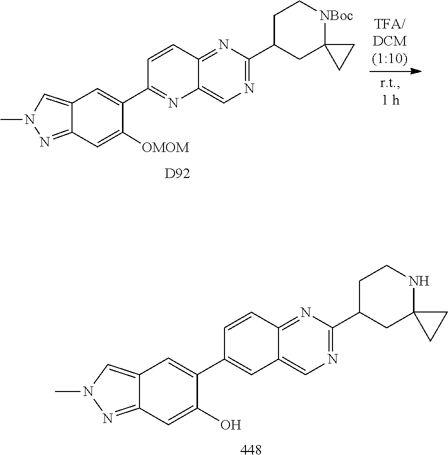

Into a 40 mL vial were added tert-butyl 7-{6-[6-(methoxymethoxy)-2-methylindazol-5-yl]pyrido [3,2-d]pyrimidin-2-yl}-4-azaspiro[2.5]octane-4-carboxylate (D92, 27 mg, 0.051 mmol, 1 equiv) and TFA (0.3 mL, 0.001 mmol, 0.02 equiv) and DCM (3 mL) at room temperature. The resulting mixture was stirred for 1 h at room temperature, basified to pH 8 with NH₃/MeOH, and subsequently concentrated under reduced pressure. The crude product (30 mg) was purified by Prep-HPLC following Condition 17, Gradient 1 as previously described to afford 5-(2-{4-azaspiro[2.5]octan-7-yl}pyrido[3,2-d]pyrimidin-6-yl)-2-methylindazol-6-ol (Compound 448, 9.5 mg). LCMS: (ES, m/z):387 [M+H]⁺. ¹H NMR: (400 MHz, DMSO-d₆) δ 12.43 (s, 1H), 9.68 (d, J=0.9 Hz, 1H), 8.75 (d, J=9.3 Hz, 1H), 8.62 (s, 1H), 8.49-8.41 (m, 2H), 6.96 (s, 1H), 4.13 (s, 3H), 3.29 (s, 1H), 3.04 (d, J=12.7 Hz, 1H), 2.82-2.71 (m, 1H), 2.22 (t, J=12.3 Hz, 1H), 2.02 (d, J=12.9 Hz, 1H), 1.79 (qd, J=12.4, 4.1 Hz, 1H), 1.42 (dd, J=12.4, 3.5 Hz, 1H), 0.56 (q, J=6.0 Hz, 1H), 0.46 (d, J=6.0 Hz, 2H), 0.40 (q, J=6.0 Hz, 1H).

### Synthesis of Compound 458

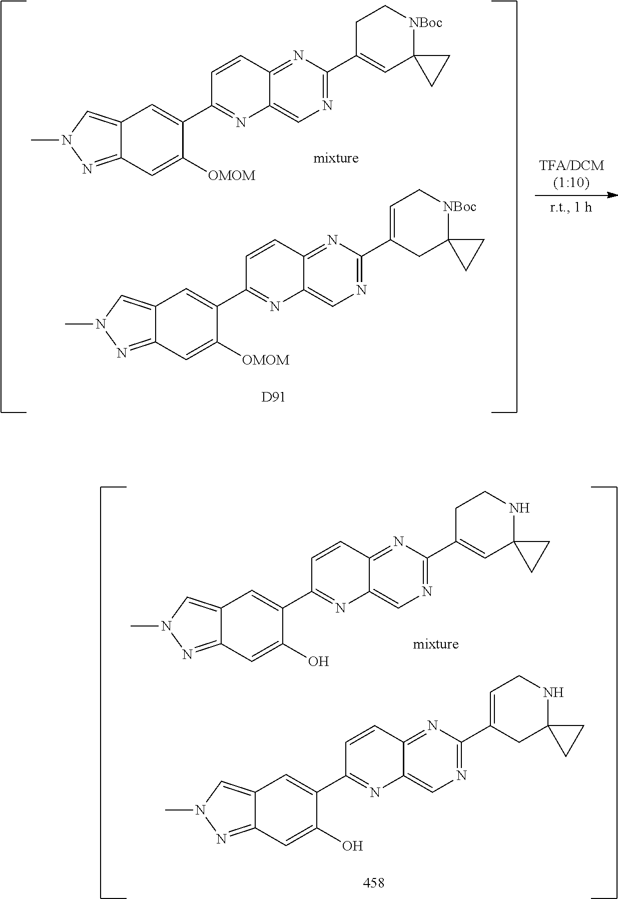

Into a 8 mL vial were added tert-butyl 7-{6-[6-(methoxymethoxy)-2-methylindazol-5-yl]pyrido[3,2-d]pyrimidin-2-yl}-4-azaspiro[2.5]oct-6-ene-4-carboxylate (D91 isomer, 50 mg, 0.095 mmol, 1 equiv) and TFA (0.3 mL, 26.926 mmol, 284.67 equiv) and DCM (3 mL) at room temperature. The resulting mixture was stirred for 2 h at room temperature, basified to pH 8 with NH₃/MeOH, and subsequently concentrated under reduced pressure. The crude product (50 mg) was purified by Prep-HPLC following Condition 16, Gradient 2 as previously delineated with the following conditions to afford 5-(2-{4-azaspiro[2.5]oct-6-en-7-yl}pyrido[3,2-d]pyrimidin-6-yl)-2-methylindazol-6-ol; 5-(6-{4-azaspiro[2.5]oct-7-en-7-yl}-1,5-naphthyridin-2-yl)-2-methylindazol-6-ol (Compound 458, 4.5 mg). LCMS: (ES, m/z):385 [M+H]⁺. ¹H NMR: (400 MHz, DMSO-d₆) δ 12.27 (s, 1H), 9.57 (d, J=15.9 Hz, 1H), 8.67 (dd, J=9.2, 3.3 Hz, 1H), 8.56 (s, 1H), 8.38 (dd, J=9.1, 2.9 Hz, 1H), 8.35 (s, 1H), 7.60-7.03 (m, 1H), 6.95 (s, 1H), 6.95 (s, 1H), 4.12 (s, 3H), 3.54 (s, 1H), 2.71-2.65 (m, 1H), 2.62 (q, J=2.4 Hz, 2H), 2.17 (s, 1H), 0.91 (t, J=2.3 Hz, 1H), 0.61-0.44 (m, 3H).

### Example 220: Synthesis of Compound 434

### Synthesis of Intermediate D94

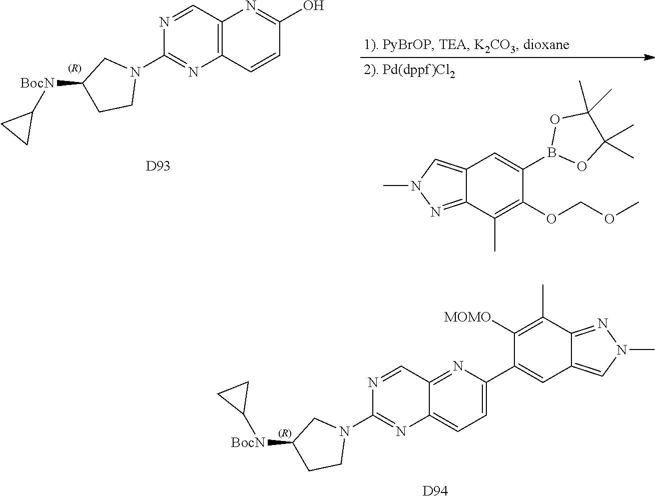

To a stirred solution of tert-butyl N-cyclopropyl-N-[(3R)-1-{6-hydroxypyrido[3,2-d]pyrimidin-2-yl}pyrrolidin-3-yl]carbamate (D93, 150 mg, 0.404 mmol, 1 equiv) and TEA (122 mg, 1.212 mmol, 3 equiv) in dioxane (10 mL) were added K₂CO₃ (167 mg, 1.212 mmol, 3 equiv) and PyBrOP (282 mg, 0.606 mmol, 1.5 equiv) at room temperature under nitrogen atmosphere. The resulting mixture was stirred for 3 h at 100° C. before the addition of Pd(dppf)Cl₂ (29 mg, 0.040 mmol, 0.1 equiv), 5-(methoxymethoxy)-2-methyl-6-(4,4,5,5-tetramethyl-1,3,2-dioxaborolan-2-yl)-1,3-benzoxazole (258 mg, 0.808 mmol, 2 equiv) and H₂O (0.5 mL) at room temperature. The resulting mixture was stirred for additional 2 h at 100° C. and subsequently purified by reverse flash chromatography following Condition 10, Gradient 1 as previously described to afford tert-butyl N-cyclopropyl-N-[(3R)-1-{7-[5-(methoxymethoxy)-2-methyl-1,3-benzoxazol-6-yl]pyrido[3,2-d]pyrimidin-2-yl}pyrrolidin-3-yl]carbamate (D94, 120 mg). LCMS: (ES, m/z): 547 [M+H]⁺.

### Synthesis of Compound 434

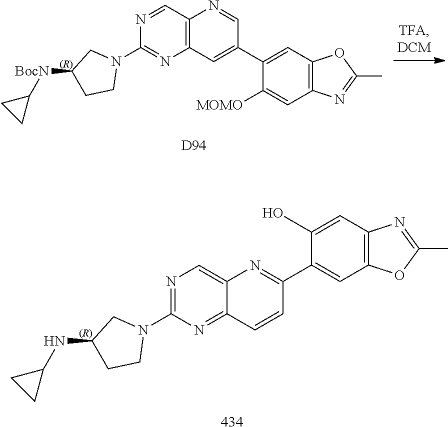

A solution of Intermediate D94 (120 mg, 0.220 mmol, 1 equiv) and trifluoroacetic acid (2 mL) in DCM (10 mL) was stirred for 3 h at room temperature and subsequently concentrated under vacuum. The residue was purified by reverse flash chromatography following Condition 10, Gradient 3 as previously described to afford 6-{2-[(3R)-3-(cyclopropylamino)pyrrolidin-1-yl]pyrido[3,2-d]pyrimidin-6-yl}-2-methyl-1,3-benzoxazol-5-ol (Compound 434, 25 mg). Absolute stereochemistry of the compound was arbitrarily assigned. LCMS: (ES, m/z): 403 [M+H]⁺. ¹H NMR: (400 MHz, DMSO-d₆) δ 13.59 (s, 1H), 9.31 (s, 1H), 8.56 (d, J=9.3 Hz, 1H), 8.39 (s, 1H), 8.06 (d, J=9.2 Hz, 1H), 7.15 (s, 1H), 3.77 (s, 1H), 3.65 (s, 1H), 3.50 (s, 2H), 3.16 (s, 2H), 2.62 (s, 3H), 2.13 (s, 2H), 1.93 (s, 1H), 0.45-0.39 (m, 2H), 0.26 (s, 2H).

### Example 221: Synthesis of Compound 432

### Synthesis of Intermediate D96

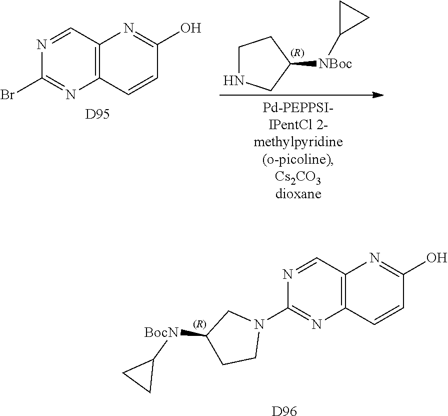

To a stirred solution of 2-bromopyrido[3,2-d]pyrimidin-6-ol (D95, 150 mg, 0.664 mmol, 1 equiv), Cs₂CO₃ (648 mg, 1.992 mmol, 3 equiv) and tert-butyl N-cyclopropyl-N-[(3R)-pyrrolidin-3-yl]carbamate (180 mg, 0.797 mmol, 1.2 equiv) in dioxane (5 mL) was added Pd-PEPPSI-IPentCl 2-methylpyridine (o-picoline) (27 mg, 0.033 mmol, 0.05 equiv) at room temperature under nitrogen atmosphere. The resulting mixture was stirred for 3 h at 100° C. and subsequently concentrated under reduced pressure. The residue was purified by reverse flash chromatography following Condition 9, Gradient 1 as previously described to afford tert-butyl N-cyclopropyl-N-[(3R)-1-{6-hydroxypyrido[3,2-d]pyrimidin-2-yl}pyrrolidin-3-yl]carbamate (D96, 150 mg). LCMS: (ES, m/z): 372 [M+H]⁺.

### Synthesis of Intermediate D97

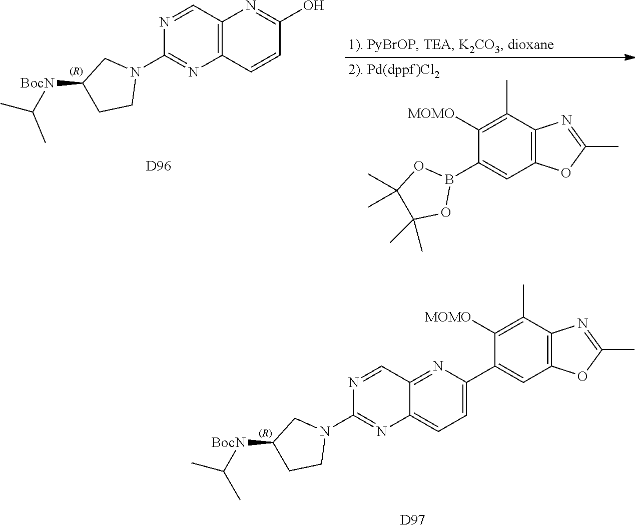

To a stirred solution of Intermediate D96 (150 mg, 0.404 mmol, 1 equiv) and TEA (122 mg, 1.212 mmol, 3 equiv) in dioxane (10 mL) were added K₂CO₃ (167 mg, 1.212 mmol, 3 equiv) and PyBrOP (282.39 mg, 0.606 mmol, 1.5 equiv) at room temperature under nitrogen atmosphere. The resulting mixture was stirred for 3 h at 100° C. before adding Pd(dppf)Cl₂ (29 mg, 0.040 mmol, 0.1 equiv), 5-(methoxymethoxy)-2,4-dimethyl-6-(4,4,5,5-tetramethyl-1,3,2-dioxaborolan-2-yl)-1,3-benzoxazole (269 mg, 0.808 mmol, 2 equiv) and H₂O (0.5 mL) at room temperature. The resulting mixture was stirred for additional 2 h at 100° C. and then purified by reverse flash chromatography following Condition 10, Gradient 1 as previously delineated to afford tert-butyl N-cyclopropyl-N-[(3R)-1-{6-[5-(methoxymethoxy)-2,4-dimethyl-1,3-benzoxazol-6-yl]pyrido[3,2-d]pyrimidin-2-yl}pyrrolidin-3-yl]carbamate (D97, 120 mg). LCMS: (ES, m/z): 561 [M+H]⁺.

### Synthesis of Compound 432

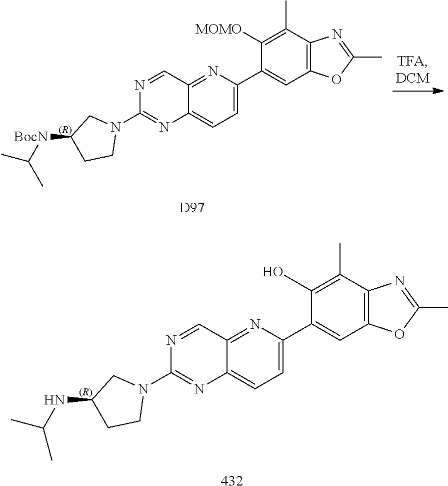

A solution of Intermediate D97 (120 mg, 0.214 mmol, 1 equiv) and trifluoroacetic acid (2 mL) in DCM (10 mL) was stirred for 3 h at room temperature, subsequently concentrated under vacuum, quenched by the addition of NH₃(g) in MeOH (2 mL) at room temperature, and concentrated under reduced pressure. The residue was purified by reverse flash chromatography following Condition 10, Gradient 3 as previously described to afford 6-{2-[(3R)-3-(cyclopropylamino)pyrrolidin-1-yl]pyrido[3,2-d]pyrimidin-6-yl}-2,4-dimethyl-1,3-benzoxazol-5-ol (Compound 432, 15 mg). Absolute stereochemistry of the compound was arbitrarily assigned. LCMS: (ES, m/z): 417 [M+H]⁺. ¹H NMR: (400 MHz, DMSO-d₆) δ 14.33 (s, 1H), 9.32 (s, 1H), 8.57 (d, J=9.3 Hz, 1H), 8.25 (s, 1H), 8.06 (d, J=9.2 Hz, 1H), 3.77 (s, 2H), 3.62 (s, 2H), 3.50 (s, 2H), 2.62 (s, 3H), 2.56 (s, 3H), 2.40 (s, 2H), 1.9-1.90 (m, 1H), 0.43 (d, J=6.7 Hz, 2H), 0.27 (s, 2H).

### Example 222: Synthesis of Compounds 495 and 496

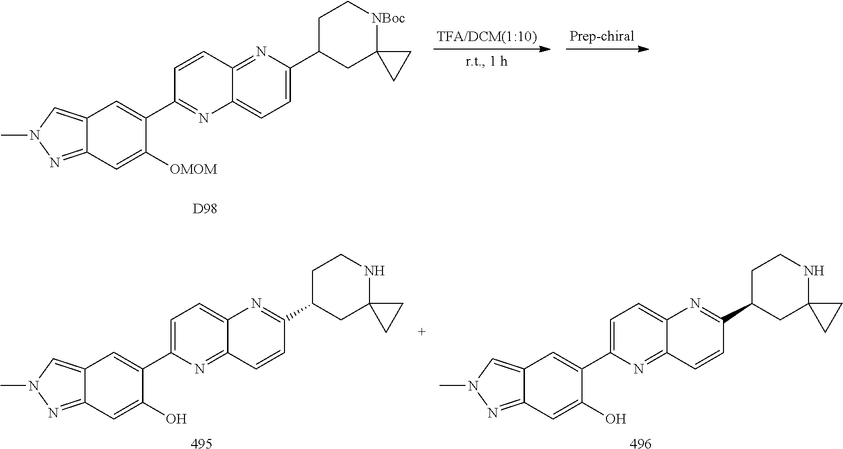

Into a 25 mL round-bottom flask were added tert-butyl 7-{6-[6-(methoxymethoxy)-2-methylindazol-5-yl]-1,5-naphthyridin-2-yl}-4-azaspiro[2.5]octane-4-carboxylate (D98, 95 mg, 0.179 mmol, 1 equiv), DCM (3 mL) and TFA (0.3 mL, 4.039 mmol, 22.52 equiv) at room temperature and allowed to stir for 1 h at room temperature. The mixture was then basified to pH 8 with NH3/MeOH and concentrated under reduced pressure. The crude product (100 mg) was purified by Prep-HPLC following Condition 18, Gradient 1 as previously described to afford 5-(6-(4-azaspiro[2.5]octan-7-yl)-1,5-naphthyridin-2-yl)-2-methyl-2H-indazol-6-ol (27 mg), which was further purified by Chiral-HPLC following Condition 16, Gradient 1 as previously delineated to afford 5-{6-[(7R)-4-azaspiro[2.5]octan-7-yl]-1,5-naphthyridin-2-yl}-2-methylindazol-6-ol (Compound 495, 5.3 mg) and 5-{6-[(7S)-4-azaspiro[2.5]octan-7-yl]-1,5-naphthyridin-2-yl}-2-methylindazol-6-ol (Compound 496, 4.6 mg). Absolute stereochemistry was assigned arbitrarily. Compound 495 was resolved from the first peak. LCMS: (ESI, m/z): 387 [M+H]. ¹H NMR: (400 MHz, DMSO-d₆) δ 13.62 (s, 1H), 8.69 (s, 1H), 8.60 (d, J=9.3 Hz, 1H), 8.47 (d, J=9.1 Hz, 1H), 8.44-8.38 (m, 2H), 7.78 (d, J=8.8 Hz, 1H), 6.92 (s, 1H), 4.13 (s, 3H), 3.29-3.18 (m, 1H), 3.07-2.99 (m, 1H), 2.75 (td, J=12.6, 2.8 Hz, 1H), 2.19 (t, J=12.3 Hz, 1H), 1.92 (d, J=12.7 Hz, 1H), 1.74 (qd, J=12.4, 4.1 Hz, 1H), 1.30 (ddd, J=12.9, 3.9, 1.7 Hz, 1H), 0.60-0.52 (m, 1H), 0.51-0.37 (m, 3H). Compound 496 was resolved from the second peak. LCMS: (ESI, m/z): 387 [M+H]. ¹H NMR: (400 MHz, DMSO-d₆) δ 13.62 (s, 1H), 8.69 (s, 1H), 8.60 (d, J=9.2 Hz, 1H), 8.47 (d, J=9.1 Hz, 1H), 8.41 (d, J=8.4 Hz, 2H), 7.77 (d, J=8.7 Hz, 1H), 6.92 (s, 1H), 4.13 (s, 3H), 3.23 (tt, J=12.0, 3.7 Hz, 1H), 3.02 (ddd, J=12.6, 4.2, 2.2 Hz, 1H), 2.75 (td, J=12.7, 2.8 Hz, 1H), 2.24-2.13 (m, 1H), 1.91 (d, J=12.7 Hz, 1H), 1.73 (qd, J=12.4, 4.1 Hz, 1H), 1.34-1.11 (m, 1H), 0.56 (td, J=8.3, 7.7, 4.0 Hz, 1H), 0.44 (tdd, J=11.9, 8.4, 5.6 Hz, 3H).

### Example 223: Synthesis of Compound 444

### Synthesis of Intermediate D100

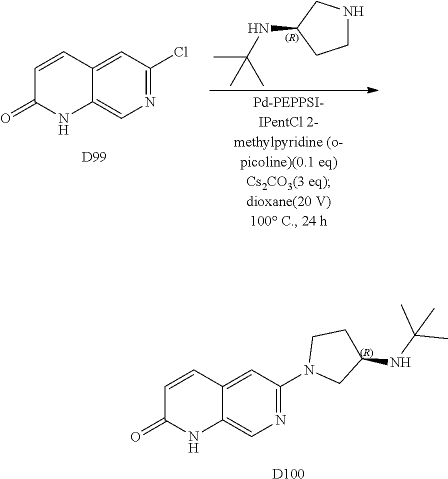

Into a 40 mL sample bottle were added 6-chloro-1H-1,7-naphthyridin-2-one (150 mg, 0.83 mmol, 1 equiv), (3R)—N-tert-butylpyrrolidin-3-amine (D99, 177.23 mg, 1.24 mmol, 1.5 equiv), Cs₂CO₃ (811.89 mg, 2.49 mmol, 3 equiv), Pd-PEPPSI-IPentCl 2-methylpyridine (o-picoline) (69.87 mg, 0.083 mmol, 0.1 equiv) and dioxane (7.5 mL). The resulting mixture was stirred for 24 h at 100° C. under nitrogen atmosphere, later allowed to cool down to room temperature, and subsequently concentrated under reduced pressure. The residue was purified by silica gel column chromatography and eluted with CH₂Cl₂/MeOH (10:1) to afford 6-[(3R)-3-(tert-butylamino)pyrrolidin-1-yl]-1H-1,7-naphthyridin-2-one (D100, 300 mg, crude). LCMS: 287[M+H]⁺.

### Synthesis of Intermediate D01

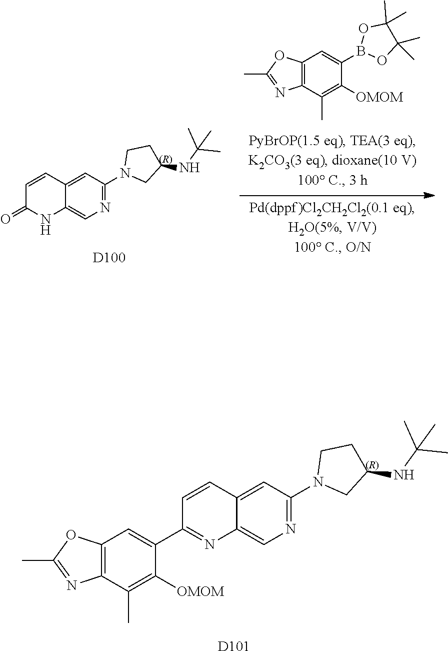

Into an 8 mL sample bottle were Intermediate D100 (100 mg, 0.34 mmol, 1 equiv), PyBrOP (244.18 mg, 0.52 mmol, 1.5 equiv), TEA (106.01 mg, 1.04 mmol, 3 equiv), K₂CO₃ (144.78 mg, 1.04 mmol, 3 equiv) and dioxane (2 mL). The resulting mixture was stirred for 3 h at 100° C. under nitrogen atmosphere. Then 5-(methoxymethoxy)-2,4-dimethyl-6-(4,4,5,5-tetramethyl-1,3,2-dioxaborolan-2-yl)-1,3-benzoxazole (116.35 mg, 0.34 mmol, 1 equiv), Pd(dppf)Cl₂CH₂Cl₂ (28.45 mg, 0.035 mmol, 0.1 equiv) and H₂O (0.1 mL) were added, and the resulting mixture was stirred for overnight at 100° C. under nitrogen atmosphere. The reaction was quenched with water (5 mL) and subsequently extracted with CH₂Cl₂ (3×10 mL). The combined organic layers were washed with a half of sat. NaCl (aq.) (2×30 mL) and sat. NaCl (aq.) (30 mL), dried over anhydrous Na₂SO₄, and the filtrate was concentrated under reduced pressure. The residue was purified by silica gel column chromatography and eluted with CH₂Cl₂/MeOH (9:1) to afford (3R)-N-tert-butyl-1-{2-[5-(methoxymethoxy)-2,4-dimethyl-1,3-benzoxazol-6-yl]-1,7-naphthyridin-6-yl}pyrrolidin-3-amine (D101,100 mg). LCMS: 476[M+H]⁺.

### Synthesis of Compound 444

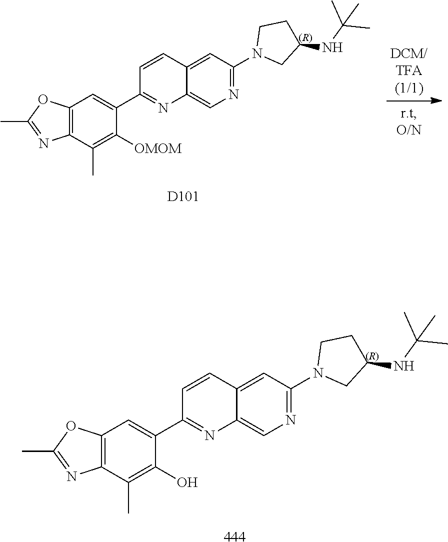

Into an 8 mL sample bottle were added Intermediate D101 (10 mg, 0.021 mmol, 1 equiv), DCM (2 mL) and TFA (2 mL), which was stirred for overnight at room temperature and subsequently concentrated under reduced pressure. The crude product was purified by Prep-HPLC following Condition 17, Gradient 2 as previously described to afford 6-{6-[(3R)-3-(tert-butylamino)pyrrolidin-1-yl]-1,7-naphthyridin-2-yl}-2,4-dimethyl-1,3-benzoxazol-5-ol (Compound 444, 9.9 mg). Absolute stereochemistry of the compound was arbitrarily assigned. LCMS: 432[M+H]. ¹H NMR: (400 MHz, DMSO-d₆) δ 14.90 (s, 1H), 9.08 (s, 1H), 8.38 (s, 1H), 8.22 (m, 2H), 6.64 (s, 1H), 3.82 (m, 1H), 3.52 (m, 1H), 3.38 (s, 1H), 3.38 (m, 1H), 3.06 (s, 1H), 2.62 (s, 3H), 2.41 (s, 3H), 2.19 (s, 1H), 1.84 (m, 1H), 1.09 (s, 9H).

### Example 224: Synthesis of Compound 445

### Synthesis of Intermediate D102

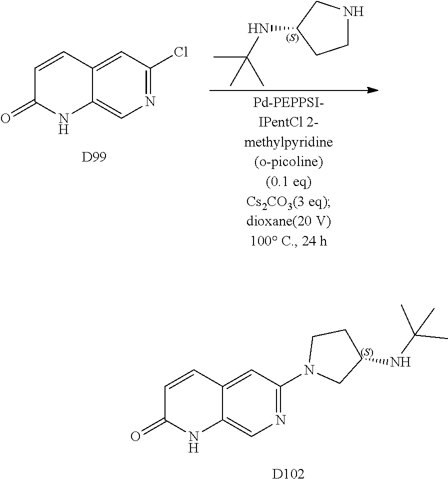

Into a 40 mL sample bottle were added Intermediate D99 (150 mg, 0.83 mmol, 1 equiv), (3S)—N-tert-butylpyrrolidin-3-amine (177.23 mg, 1.24 mmol, 1.5 equiv), Cs₂CO₃ (811.89 mg, 2.49 mmol, 3 equiv), Pd-PEPPSI-IPentCl 2-methylpyridine (o-picoline) (69.87 mg, 0.083 mmol, 0.1 equiv) and dioxane (7.5 mL). The resulting mixture was stirred for 24 h at 100° C. under nitrogen atmosphere, then allowed to cool to room temperature, and subsequently concentrated under reduced pressure. The residue was purified by silica gel column chromatography and eluted with CH₂Cl₂/MeOH (10:1) to afford 6-[(3S)-3-(tert-butylamino)pyrrolidin-1-yl]-1H-1,7-naphthyridin-2-one (D102, 200 mg). LCMS: 287[M+H]⁺.

### Synthesis of Intermediate D103

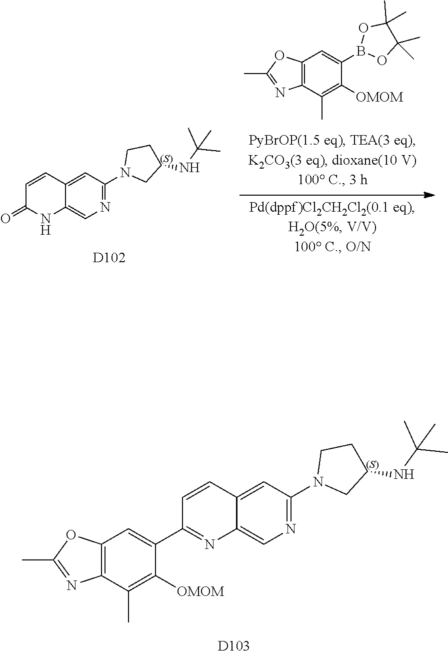

Into an 8 mL sample bottle were Intermediate D102 (180 mg, 0.62 mmol, 1 equiv), PyBrOP (439.52 mg, 0.94 mmol, 1.5 equiv), TEA (190.81 mg, 1.88 mmol, 3 equiv), K₂CO₃ (260.6 mg, 1.88 mmol, 3 equiv) and dioxane (4 mL). The resulting mixture was stirred for 3 h at 100° C. under nitrogen atmosphere. Then 5-(methoxymethoxy)-2,4-dimethyl-6-(4,4,5,5-tetramethyl-1,3,2-dioxaborolan-2-yl)-1,3-benzoxazole (209.42 mg, 0.62 mmol, 1 equiv), Pd(dppf)Cl₂CH₂Cl₂ (45.99 mg, 0.063 mmol, 0.1 equiv) and H₂O (0.2 mL) were added, and the resulting mixture was stirred for overnight at 100° C. under nitrogen atmosphere. The reaction was quenched with water (5 mL) and then extracted with CH₂Cl₂ (3×10 mL). The combined organic layers were washed with a half of sat. NaCl (aq.) (2×30 mL) and sat. NaCl (aq.) (30 mL), dried over anhydrous Na₂SO₄, and the filtrate was concentrated under reduced pressure. The residue was purified by silica gel column chromatography and eluted with CH₂Cl₂/MeOH (9:1) to afford (3S)—N-tert-butyl-1-{2-[5-(methoxymethoxy)-2,4-dimethyl-1,3-benzoxazol-6-yl]-1,7-naphthyridin-6-yl}pyrrolidin-3-amine (D103, 450 mg).

### Synthesis of Compound 445

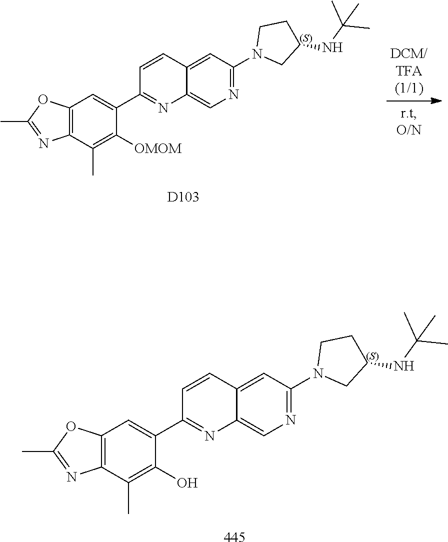

Into a 40 mL sample bottle were added Intermediate D103 (200 mg, 0.16 mmol, 1 equiv), DCM (4 mL) and TFA (4 mL), which was stirred overnight at room temperature and later concentrated under reduced pressure. The crude product was purified by Prep-HPLC following Condition 11, Gradient 4 as previously described to afford 6-{6-[(3S)-3-(tert-butylamino)pyrrolidin-1-yl]-1,7-naphthyridin-2-yl}-2,4-dimethyl-1,3-benzoxazol-5-ol (Compound 445, 20.6 mg). Absolute stereochemistry of the compound was arbitrarily assigned. LCMS: (ES, m/z): 432[M+H]. ⁺. ¹H NMR: (400 MHz, DMSO-d₆) δ 14.90 (s, 1H), 9.08 (s, 1H), 8.38 (d, J=9.4 Hz, 1H),8.22 (m, 2H), 6.65 (s, 1H), 3.78 (dd, J=10.0, 6.8 Hz, 1H), 3.59 (dt, J=21.7, 8.4 Hz, 2H), 3.47 (m, 1H), 3.07 (dd, J=10.2, 7.2 Hz, 1H), 2.62 (s, 3H), 2.41 (s, 3H), 2.16 (m, 1H), 1.86 (m, 1H), 1.10 (s, 9H).

### Example 225: Synthesis of Compounds 497-500

### Synthesis of Intermediate D05

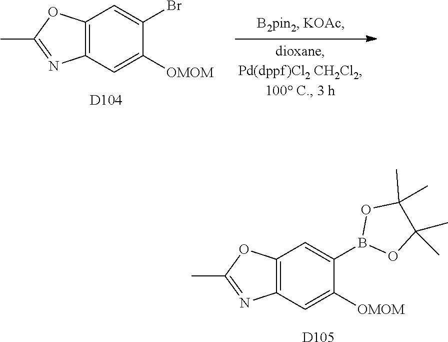

Into a 250 mL 3-necked round-bottom flask were added Intermediate D104 (5 g, 18.376 mmol, 1 equiv), KOAc (5.41 g, 55.128 mmol, 3 equiv), dioxane (100 mL), and Pd(dppf)Cl₂·CH₂Cl₂ (1.50 g, 1.838 mmol, 0.1 equiv) at room temperature. The resulting mixture was stirred for 3 h at 100° C. under nitrogen atmosphere and subsequently allowed to cool to room temperature. The resulting mixture was filtered, washed with EtOAc (3×10 mL), and the filtrate was concentrated under reduced pressure. The residue was purified by silica gel column chromatography and eluted with PE/EA (3:1) to afford 5-(methoxymethoxy)-2-methyl-6-(4,4,5,5-tetramethyl-1,3,2-dioxaborolan-2-yl)-1,3-benzoxazole (D105, 4.8 g). LCMS: (ESI, m/z): 320 [M+H]

### Synthesis of Intermediate D106

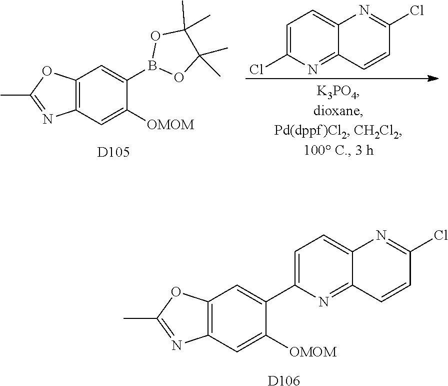

Into a 20 mL vial were added Intermediate D105 (400 mg, 1.253 mmol, 1 equiv),2,6-dichloro-1,5-naphthyridine (249.44 mg, 1.253 mmol, 1 equiv), dioxane (8 mL), K₃PO₄ (798.09 mg, 3.759 mmol, 3 equiv) in H₂O (1.6 mL) and Pd(dppf)Cl₂·CH₂Cl₂ (91.7 mg, 0.125 mmol, 0.1 equiv) at room temperature. The resulting mixture was stirred for 3 h at 100° C. under nitrogen atmosphere, then allowed to cool to room temperature, and subsequently concentrated under reduced pressure. The residue was purified by silica gel column chromatography and eluted with CH₂Cl₂/EA (3:2) to afford 2-chloro-6-[5-(methoxymethoxy)-2-methyl-1,3-benzoxazol-6-yl]-1,5-naphthyridine (D106, 250 mg). LCMS: (ESI, m/z): 356 [M+H].

### Synthesis of Intermediate D107

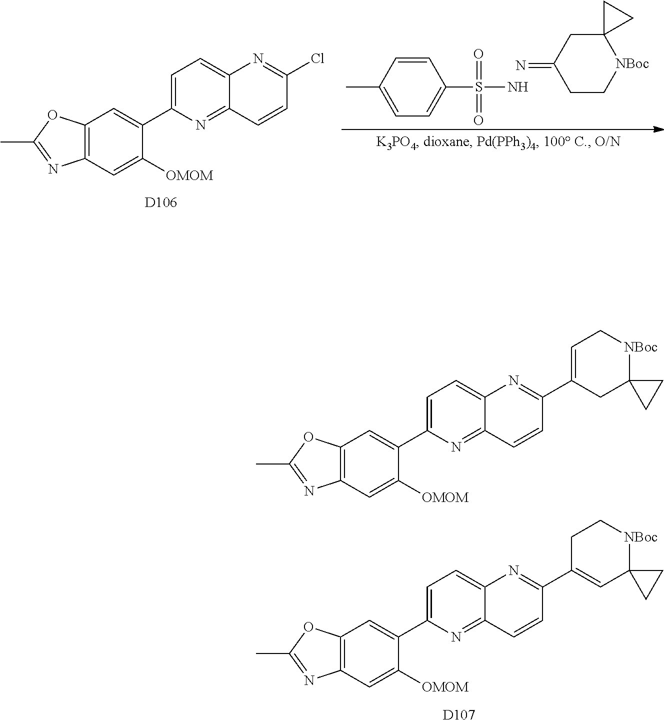

Into a 40 mL vial were added Intermediate D106 (400 mg, 1.124 mmol, 1 equiv), dioxane (12 mL, 141.648 mmol, 125.99 equiv), K₃PO₄ (715.94 mg, 3.372 mmol, 3 equiv) and Pd(PPh₃)4 (129.92 mg, 0.112 mmol, 0.1 equiv) at room temperature. The resulting mixture was stirred for overnight at 100° C. under nitrogen atmosphere. The mixture was allowed to cool to room temperature, quenched by the addition of water (10 mL) at room temperature, and lastly concentrated under reduced pressure. The residue was then purified by silica gel column chromatography and eluted with PE/EA (1:3) to afford mixture product (D107, 220 mg) as a yellow solid. LCMS: (ESI, m/z): 529 [M+H].

### Synthesis of Intermediate D108

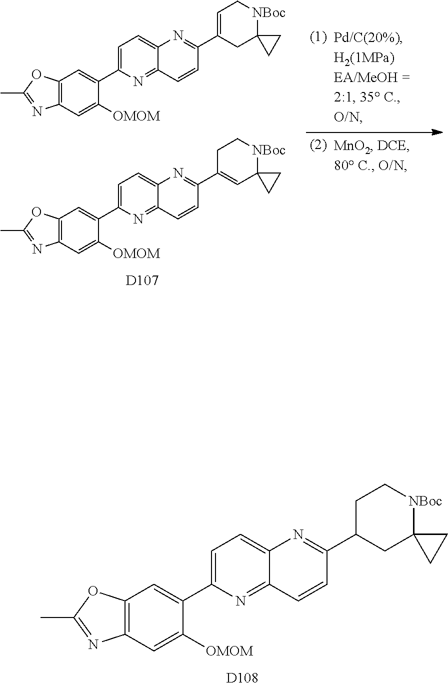

Into a 25 mL round-bottom flask were added the mixture tert-butyl 7-{6-[5-(methoxymethoxy)-2-methyl-1,3-benzoxazol-6-yl]-1,5-naphthyridin-2-yl}-4-azaspiro[2.5]oct-6-ene-4-carboxylate and tert-butyl 7-{6-[5-(methoxymethoxy)-2-methyl-1,3-benzoxazol-6-yl]-1,5-naphthyridin-2-yl}-4-azaspiro[2.5]oct-7-ene-4-carboxylate (D107, 180 mg, 0.170 mmol, 1 equiv), MeOH (1 mL, 24.699 mmol, 145.07 equiv) and Pd/C (5.44 mg, 0.051 mmol, 0.3 equiv) at room temperature. The resulting mixture was stirred overnight at 35° C. under hydrogen atmosphere. Trace desired product was detected by LCMS. The resulting mixture was filtered and the filter cake was washed with DCM and MeOH (2:1, 3×3 mL), then concentrated under reduced pressure. The crude mixture in DCE (5 mL) was treated with MnO₂ (148.02 mg, 1.700 mmol, 10 equiv) overnight at 80° C. and subsequently allowed to cool to room temperature. The resulting mixture was filtered, washed with DCM and MeOH (2:1, 3×3 mL), and the filtrate was concentrated under reduced pressure. The residue was purified by Prep-TLC (CH₂Cl₂/MeOH 10:1) to afford tert-butyl 7-{6-[5-(methoxymethoxy)-2-methyl-1,3-benzoxazol-6-yl]-1,5-naphthyridin-2-yl}-4-azaspiro[2.5]octane-4-carboxylate (D108, 80 mg). LCMS: (ESI, m/z): 531 [M+H].

### Synthesis of Compounds 497 and 498

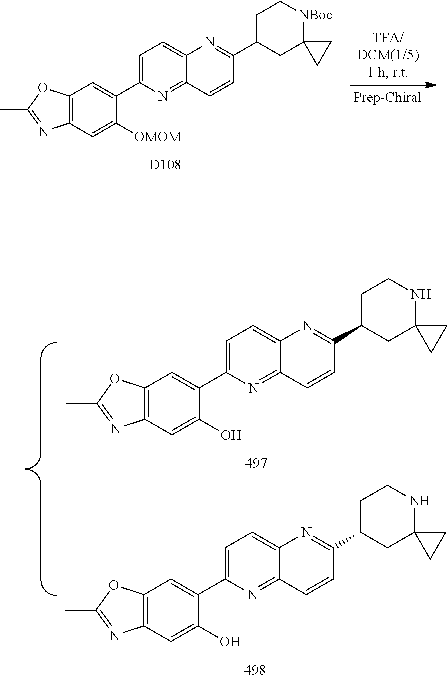

Into a 20 mL vial were added Intermediate D108 (85 mg, 0.160 mmol, 1 equiv), DCM (5 mL) and TFA (0.5 mL) at room temperature. The resulting mixture was stirred for 1 h at room temperature, basified to pH 8 with NH₃/MeOH, and then concentrated under reduced pressure. The crude product (80 mg) was purified by Prep-HPLC following Condition 11, Gradient 5 as previously described to afford 6-(6-(4-azaspiro[2.5]octan-7-yl)-1,5-naphthyridin-2-yl)-2-methylbenzo[d]oxazol-5-ol (25 mg), which was purified by Chiral-HPLC following Condition 17, Gradient 1 as previously described to afford 6-{6-[(7S)-4-azaspiro[2.5]octan-7-yl]-1,5-naphthyridin-2-yl}-2-methyl-1,3-benzoxazol-5-ol (Compound 497, 9.9 mg) and 6-{6-[(7R)-4-azaspiro[2.5]octan-7-yl]-1,5-naphthyridin-2-yl}-2-methyl-1,3-benzoxazol-5-ol (Compound 498, 9.4 mg). Compounds 497 and 498 were resolved from the first and second peaks, respectively. Absolute stereochemistry of the compounds was arbitrarily assigned. Analytical data for the respective compounds are shown in the Table below.

### Synthesis of Compounds 499 and 500

As would be manifest to a person of skill in the art, Compounds 499 and 500 (shown in the Table below) were synthesized following a similar synthetic scheme as described above in this Example. Compounds 499 and 500 were resolved from the first and second peaks, respectively, by Chiral HPLC. Absolute stereochemistry of the compounds was assigned arbitrarily.

| Compound Structure | Analysis data |
|---|---|
| 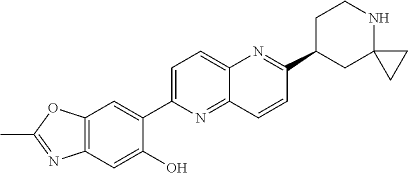 [SMILES: Cc1nc2cc(O)c(-c3ccc4nc([C@H]5CCNC6(CC6)C5)ccc4n3)cc2o1] | LCMS: (ESI, m/z): 387 [M + H]. ¹H NMR: (400 MHz, DMSO-d₆) δ 14.35 (s, 1H), 8.65 (d, J = 9.3 Hz, 1H), 8.56 (s, 1H), 8.51 (dd, J = 9.1, 0.9 Hz, 1H), 8.45 (dd, J = 8.7, 0.8 Hz, 1H), 7.80 (d, J = 8.7 Hz, 1H), 7.18 (s, 1H), 3.33-3.17 (m, 1H), 3.06- 2.97 (m, 1H), 2.82-2.69 (m, 1H), 2.63 (s, 3H), 2.19 (td, J = 12.4, 1.6 Hz, 1H), 1.91 (d, J = 12.6 Hz, 1H), 1.73 (qd, J = 12.5, 4.2 Hz, 1H), 1.34-1.21 (m, 1H), 0.55 (td, J = 8.3, 7.4, 4.2 Hz, 1H), 0.50-0.36 (m, 3H). |
| 497 | |
| 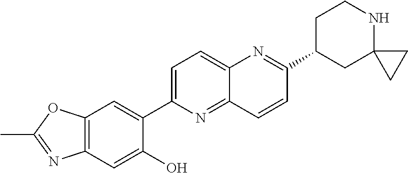 [SMILES: Cc1nc2cc(O)c(-c3ccc4nc([C@@H]5CCNC6(CC6)C5)ccc4n3)cc2o1] | LCMS: (ESI, m/z): 387 [M + H]. ¹H NMR: (400 MHz, DMSO-d₆) δ 14.34 (s, 1H), 8.65 (d, J = 9.3 Hz, 1H), 8.56 (s, 1H), 8.51 (d, J = 9.1 Hz, 1H), 8.48-8.41 (m, 1H), 7.80 (d, J = 8.7 Hz, 1H), 7.18 (s, 1H), 3.29-3.18 (m, 1H), 3.06-2.98 (m, 1H), 2.80-2.69 (m, 1H), 2.63 (s, 3H), 2.24-2.13 (m, 1H), 1.92 (d, J = 12.7 Hz, 1H), 1.73 (qd, J = 12.4, 4.1 Hz, 1H), 1.30 (ddd, J = 12.7, 4.0, 1.7 Hz, 1H), 0.56 (td, J = 8.4, 7.6, 4.2 Hz, 1H), 0.44 (tq, J = 10.6, 3.1 Hz, 3H). |
| 498 | |
| 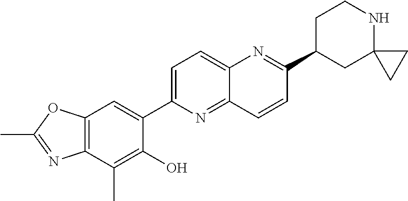 [SMILES: Cc1nc2c(C)c(O)c(-c3ccc4nc([C@H]5CCNC6(CC6)C5)ccc4n3)cc2o1] | LCMS: (ES, m/z): 400 [M + H] ⁺. ¹H NMR: (400 MHz, DMSO-d₆) δ 14.93 (d, J = 2.6 Hz, 1H), 8.64 (d, J = 9.4 Hz, 1H), 8.48 (dd, J = 16.6, 8.9 Hz, 2H), 8.39 (s, 1H), 7.80 (d, J = 8.7 Hz, 1H), 3.07 (d, J = 12.7 Hz, 1H), 2.89 (q, J = 7.3 Hz, 1H), 2.79 (t, J = 12.3 Hz, 1H), 2.63 (s, 3H), 2.42 (s, 3H), 2.22 (t, J = 12.4 Hz, 1H), 1.95 (d, J = 12.9 Hz, 1H), 1.85-1.70 (m, 1H), 1.33 (d, J = 12.9 Hz, 1H), 0.67- 0.57 (m, 1H), 0.47 (q, J = 13.0, 10.5 Hz, 3H). |
| 499 | |
| 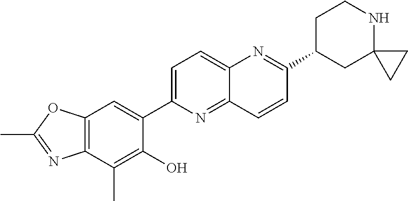 [SMILES: Cc1nc2c(C)c(O)c(-c3ccc4nc([C@@H]5CCNC6(CC6)C5)ccc4n3)cc2o1] | LCMS: (ES, m/z): 400 [M + H] ⁺ ¹H NMR: (400 MHz, DMSO-d₆) δ 14.95 (s, 1H), 8.65 (d, J = 9.2 Hz, 1H), 8.48 (dd, J = 14.5, 8.9 Hz, 2H), 8.42-8.37 (m, 1H), 7.80 (d, J = 8.7 Hz, 1H), 3.29- 3.19 (m, 1H), 3.10 (d, J = 12.7 Hz, 1H), 2.81 (dd, J = 13.7, 10.9 Hz, 1H), 2.63 (s, 3H), 2.42 (s, 3H), 2.24 (t, J = 12.4 Hz, 1H), 1.97 (d, J = 12.1 Hz, 1H), 1.84- 1.72 (m, 1H), 1.41-1.29 (m, 1H), 0.64 (d, J = 8.7 Hz, 1H), 0.59-0.44 (m, 3H). |
| 500 | |

### Example 226: Synthesis of Compound 504

### Synthesis of Intermediate D10

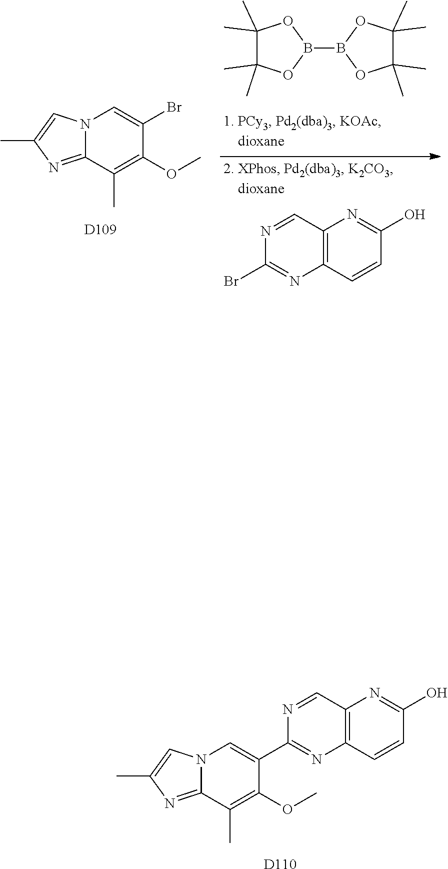

To a stirred solution of 6-bromo-7-methoxy-2,8-dimethylimidazo[1,2-a]pyridine (D109, 2.5 g, 9.8 mmol, 1 equiv), KOAc (1.93 g, 19.6 mmol, 2 equiv) and bis(pinacolato)diboron (3.73 g, 14.7 mmol, 1.5 equiv) in dioxane (40 mL) were added PCy₃ (1.65 g, 5.88 mmol, 0.6 equiv) and Pd₂(dba)₃ (2.69 g, 2.94 mmol, 0.3 equiv) at room temperature under nitrogen atmosphere. The resulting mixture was stirred for 3 h at 100° C. under nitrogen atmosphere and subsequently allowed to cool to room temperature. To the above mixture were added 2-bromopyrido[3,2-d]pyrimidin-6-ol (2.22 g, 9.8 mmol, 1 equiv), K₂CO₃ (2.72 g, 19.6 mmol, 2 equiv), XPhos (2.81 g, 5.88 mmol, 0.6 equiv) and Pd₂(dba)₃ (2.69 g, 2.94 mmol, 0.3 equiv) at room temperature under nitrogen atmosphere. The resulting mixture was stirred for additional 3 h at 100° C., then allowed to cool to room temperature, and finally concentrated under vacuum. The residue was purified by reversed-phase flash chromatography following Condition 7, Gradient 7 as previously described to afford 2-{7-methoxy-2,8-dimethylimidazo[1,2-a]pyridin-6-yl}pyrido[3,2-d]pyrimidin-6-ol (D110, 400 mg). LCMS: (ES, m/z): 322 [M+H]⁺.

### Synthesis of D111

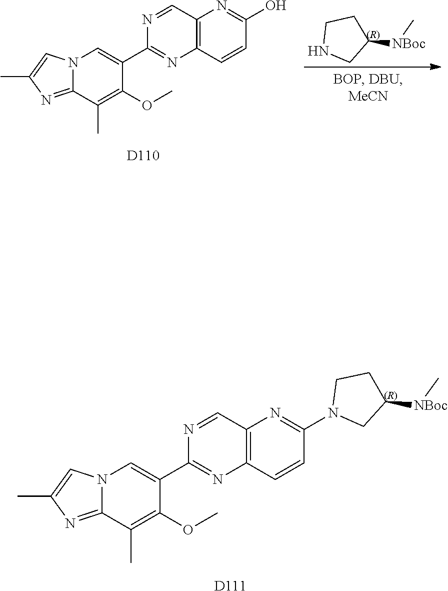

To a stirred solution of Intermediate D110 (300 mg, 0.934 mmol, 1 equiv) and tert-butyl N-methyl-N-[(3R)-pyrrolidin-3-yl]carbamate (281 mg, 1.401 mmol, 1.5 equiv) in ACN (5 mL) were added BOP (826 mg, 1.868 mmol, 2 equiv) and DBU (427 mg, 2.802 mmol, 3 equiv) at room temperature. The resulting mixture was stirred for overnight at room temperature and later concentrated under reduced pressure. The residue was purified by reversed-phase flash chromatography following Condition 10, Gradient 11 as previously described to afford tert-butyl N-[(3R)-1-(2-{7-methoxy-2,8-dimethylimidazo[1,2-a]pyridin-6-yl}pyrido[3,2-d]pyrimidin-6-yl)pyrrolidin-3-yl]-N-methylcarbamate (D111, 280 mg). LCMS: (ES, m/z): 504 [M+H]⁺.

### Synthesis of Compound 504

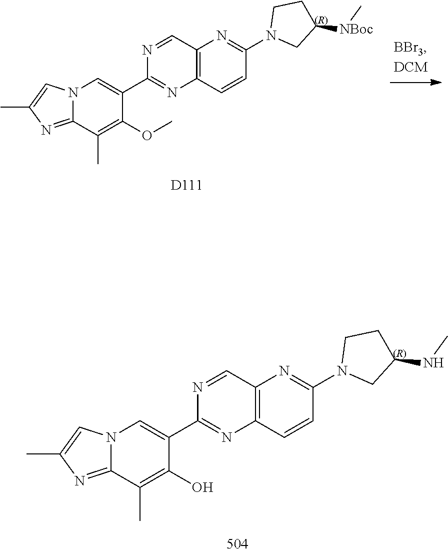

To a stirred solution of Intermediate D111 (300 mg, 0.596 mmol, 1 equiv) in DCE (5 mL) was added BBr₃ (157 mg, 0.626 mmol, 1.05 equiv) at room temperature. The resulting mixture was stirred for 1 h at 70° C., subsequently allowed to cool to room temperature, and lastly concentrated under vacuum. The residue was purified by reversed-phase flash chromatography following Condition 10, Gradient 27 as previously described to afford 2,8-dimethyl-6-{6-[(3R)-3-(methylamino)pyrrolidin-1-yl]pyrido[3,2-d]pyrimidin-2-yl}imidazo[1,2-a]pyridin-7-ol (Compound 504, 60 mg). Absolute stereochemistry of the compound was arbitrarily assigned. LCMS: (ES, m/z): 390 [M+H]⁺. ¹H NMR: (300 MHz, DMSO-d₆) δ 13.19 (s, 1H), 9.35 (s, 1H), 9.20 (s, 1H), 8.13 (d, J=9.4 Hz, 1H), 7.64 (s, 1H), 7.37 (d, J=9.4 Hz, 1H), 3.66 (d, J=5.6 Hz, 3H), 3.32 (s, 1H), 2.41-2.21 (m, 9H), 2.23-1.77 (m, 3H).

### Example 227: Synthesis of Compound 505

### Synthesis of Intermediate D113

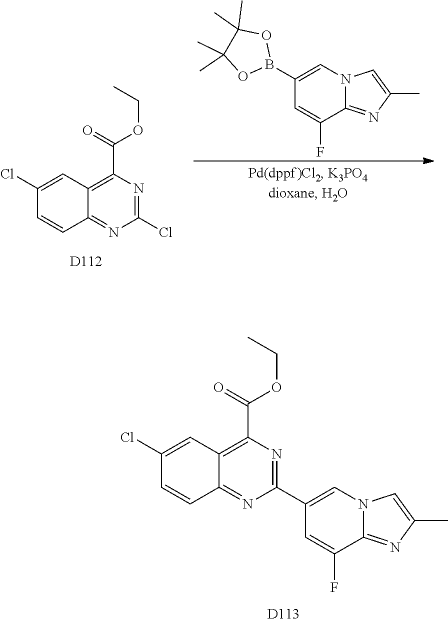

To a stirred mixture of ethyl 2,6-dichloroquinazoline-4-carboxylate (D112, 200 mg, 0.738 mmol, 1 equiv) and 8-fluoro-2-methyl-6-(4,4,5,5-tetramethyl-1,3,2-dioxaborolan-2-yl) imidazo[1,2-a]pyridine (244.44 mg, 0.886 mmol, 1.2 equiv) in dioxane (3 mL) and H₂O (0.6 mL) were added K₃PO₄ (234.89 mg, 1.107 mmol, 1.5 equiv) and Pd(dppf)Cl₂ (53.98 mg, 0.074 mmol, 0.1 equiv) at room temperature under nitrogen atmosphere. The resulting mixture was stirred for 4 h at 80° C. under nitrogen atmosphere and then concentrated under reduced pressure. The residue was purified by silica gel column chromatography and eluted with PE/EA (1:1) to afford ethyl 6-chloro-2-{8-fluoro-2-methylimidazo[1,2-a]pyridin-6-yl}quinazoline-4-carboxylate (D113, 160 mg). LCMS: (ES, m/z): 385 [M+H]⁺.

### Synthesis of Intermediate D114

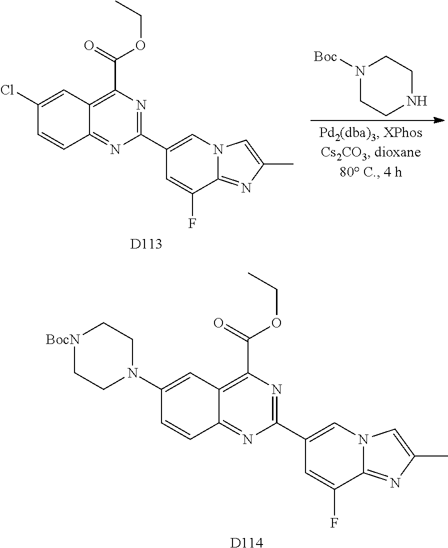 [SMILES: CCOC(=O)c1nc(-c2cc(F)c3nc(C)cn3c2)nc2ccc(Cl)cc12 ; CC(C)(C)OC(=O)N1CCNCC1 ; CCOC(=O)c1nc(-c2cc(F)c3nc(C)cn3c2)nc2ccc(N3CCN(C(=O)OC(C)(C)C)CC3)cc12 ; "D113" ; "D114"]

To a stirred mixture of Intermediate D113 (160 mg, 0.416 mmol, 1 equiv) and tert-butyl piperazine-1-carboxylate (100.68 mg, 0.541 mmol, 1.3 equiv) in dioxane (3 mL) were added Cs₂CO₃ (406.43 mg, 1.248 mmol, 3 equiv), X-Phos (39.64 mg, 0.083 mmol, 0.2 equiv) and Pd₂(dba)₃ (38.08 mg, 0.042 mmol, 0.1 equiv) at room temperature under nitrogen atmosphere. The resulting mixture was stirred for 4 h at 80° C. under nitrogen atmosphere and subsequently concentrated under reduced pressure. The residue was purified by silica gel column chromatography and eluted with DCM/MeOH (20:1) to afford ethyl 6-[4-(tert-butoxycarbonyl) piperazin-1-yl]-2-{8-fluoro-2-methylimidazo[1,2-a]pyridin-6-yl}quinazoline-4-carboxylate (D114, 180 mg). LCMS: (ES, m/z): 535 [M+H]⁺.

### Synthesis of Intermediate D115

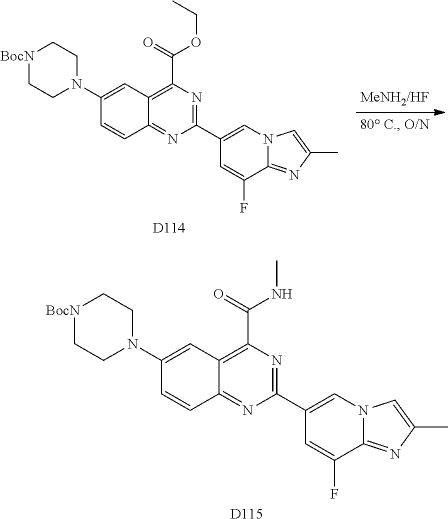 [SMILES: CCOC(=O)c1nc(-c2cc(F)c3nc(C)cn3c2)nc2ccc(N3CCN(C(=O)OC(C)(C)C)CC3)cc12 ; CNC(=O)c1nc(-c2cc(F)c3nc(C)cn3c2)nc2ccc(N3CCN(C(=O)OC(C)(C)C)CC3)cc12 ; "D114" ; "D115"]

Intermediate D114 (180 mg, 0.337 mmol, 1 equiv) was added to a stirred solution of 2.0 M methylamine in THE (8 mL) at room temperature. The resulting mixture was stirred overnight at 80° C. and then concentrated under reduced pressure to afford tert-butyl 4-(2-{8-fluoro-2-methylimidazo[1,2-a]pyridin-6-yl}-4-(methylcarbamoyl) quinazolin-6-yl) piperazine-1-carboxylate (D115, 49 mg). LCMS: (ES, m/z): 520 [M+H]⁺.

### Synthesis of Compound 505

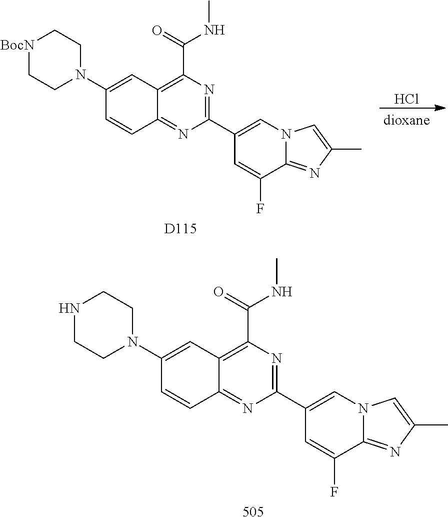 [SMILES: CNC(=O)c1nc(-c2cc(F)c3nc(C)cn3c2)nc2ccc(N3CCN(C(=O)OC(C)(C)C)CC3)cc12 ; Cl ; CNC(=O)c1nc(-c2cc(F)c3nc(C)cn3c2)nc2ccc(N3CCNCC3)cc12 ; "D115" ; "505"]

To a stirred solution of Intermediate D115 (49 mg, 0.094 mmol, 1 equiv) in dioxane (0.5 mL) was added HCl (gas) in 1,4-dioxane (0.5 mL) at room temperature. The resulting mixture was stirred for 1 h at room temperature, concentrated under reduced pressure, and the crude product was purified by Prep-HPLC following Condition 13, Gradient 3 as previously described to afford 2-{8-fluoro-2-methylimidazo[1,2-a]pyridin-6-yl}-N-methyl-6-(piperazin-1-yl) quinazoline-4-carboxamide (Compound 505, 10.8 mg). LCMS: (ES, m/z): 420 [M+H]⁺. ¹H NMR: (400 MHz, Methanol-d₄) δ 9.43 (d, J=1.3 Hz, 1H), 8.41 (d, J=2.7 Hz, 1H), 8.10-7.98 (m, 2H), 7.92 (dd, J=9.4, 2.8 Hz, 1H), 7.82 (d, J=2.8 Hz, 1H), 3.71-3.61 (m, 4H), 3.49 (dd, J=6.8, 3.8 Hz, 4H), 3.11 (s, 3H), 2.49 (s, 3H).

### Example 228: Synthesis of Compound 507

### Synthesis of Intermediate D116

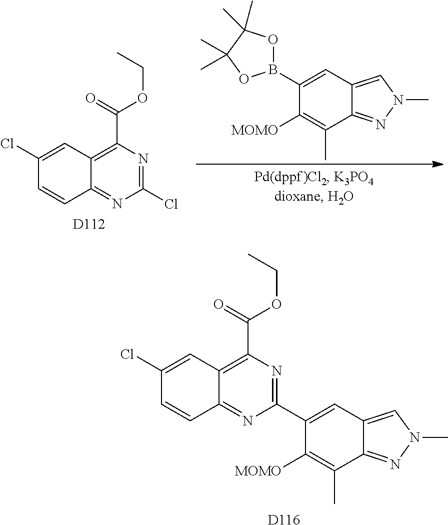

To a stirred mixture of Intermediate D112 (210 mg, 0.775 mmol, 1 equiv) and 6-(methoxymethoxy)-2,7-dimethyl-5-(4,4,5,5-tetramethyl-1,3,2-dioxaborolan-2-yl) indazole (283.07 mg, 0.853 mmol, 1.1 equiv) in dioxane/H₂O (5:1; 3.6 mL) were added K₃PO₄ (246.64 mg, 1.163 mmol, 1.5 equiv) and Pd(dppf)Cl₂ (56.68 mg, 0.078 mmol, 0.1 equiv) at room temperature under nitrogen atmosphere. The resulting mixture was stirred for 4 h at 80° C. under nitrogen atmosphere and then concentrated under reduced pressure. The residue was purified by silica gel column chromatography and eluted with PE/EA (1:1) to afford ethyl 6-chloro-2-[6-(methoxymethoxy)-2,7-dimethylindazol-5-yl]quinazoline-4-carboxylate (D116, 230 mg). LCMS: (ES, m/z): 441 [M+H]⁺.

### Synthesis of Intermediate D117

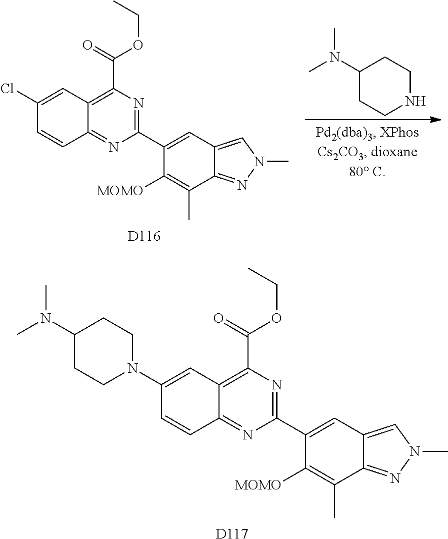

To a stirred mixture of Intermediate D116 (230 mg, 0.522 mmol, 1 equiv) and N,N-dimethylpiperidin-4-amine (86.96 mg, 0.679 mmol, 1.3 equiv) in dioxane (4.5 mL) were added Cs₂CO₃ (509.92 mg, 1.566 mmol, 3 equiv), X-Phos (49.74 mg, 0.104 mmol, 0.2 equiv) and Pd₂(dba)₃ (47.77 mg, 0.052 mmol, 0.1 equiv) at room temperature under nitrogen atmosphere. The resulting mixture was stirred for 4 h at 80° C. under nitrogen atmosphere and subsequently concentrated under reduced pressure. The residue was purified by silica gel column chromatography and eluted with DCM/MeOH (20:1) to afford ethyl 6-[4-(dimethylamino)piperidin-1-yl]-2-[6-(methoxymethoxy)-2,7-dimethylindazol-5-yl]quinazoline-4-carboxylate (D117, 200 mg). LCMS: (ES, m/z): 533 [M+H]⁺.

### Synthesis of Intermediate D118

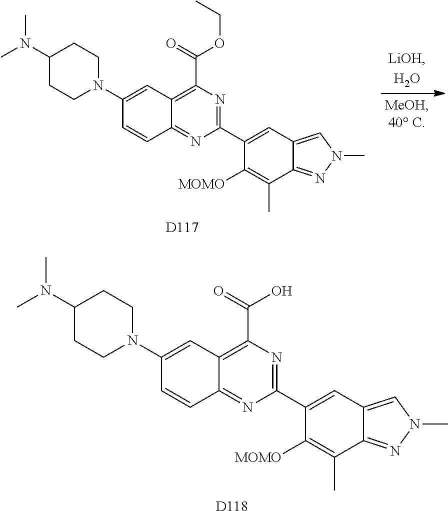

To a stirred solution of Intermediate D117 (200 mg, 0.375 mmol, 1 equiv) in MeOH (2 mL) was added LiOH H₂O (31.51 mg, 0.750 mmol, 2 equiv) at room temperature. The resulting mixture was stirred overnight at 40° C., concentrated under vacuum, diluted with H₂O (5 mL), and then acidified to pH 6 with HCl (1N). The aqueous layer was extracted with DCM (3×100 mL), and the combined organic layers were concentrated under reduced pressure to afford 6-[4-(dimethylamino)piperidin-1-yl]-2-[6-(methoxymethoxy)-2,7-dimethylindazol-5-yl]quinazoline-4-carboxylic acid (D118, 150 mg). LCMS: (ES, m/z): 505 [M+H]⁺.

### Synthesis of Intermediate D119

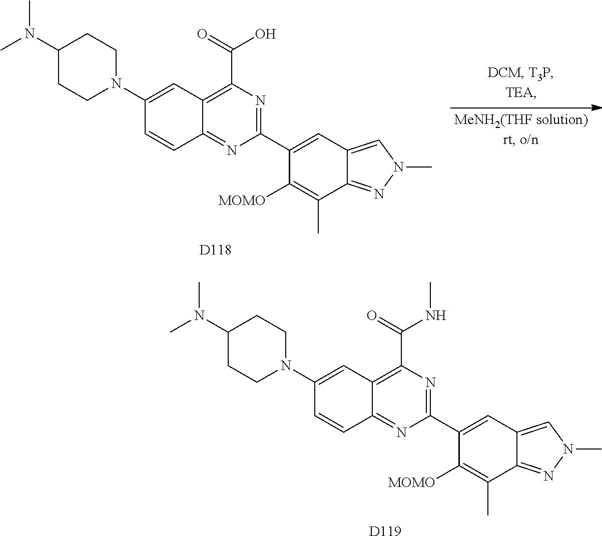

To a stirred mixture of Intermediate D118 (150 mg, 0.286 mmol, 1 equiv) and methylamine (2M in THF) (9.77 mg, 0.315 mmol, 1.1 equiv) in DCM (3 mL) was added T₃P (118.24 mg, 0.372 mmol, 1.3 equiv) and TEA (86.78 mg, 0.858 mmol, 3 equiv) at room temperature. The resulting mixture was stirred overnight at room temperature and then concentrated under reduced pressure. The residue was purified by silica gel column chromatography and eluted with DCM/MeOH (10:1) to afford {6-[4-(dimethylamino)piperidin-1-yl]-2-[6-(methoxymethoxy)-2,7-dimethyl-octahydroindazol-5-yl]-decahydroquinazolin-4-yl}(methylamino)methanol (D119, 15 mg). LCMS: (ES, m/z): 518 [M+H]⁺.

### Synthesis of Compound 507

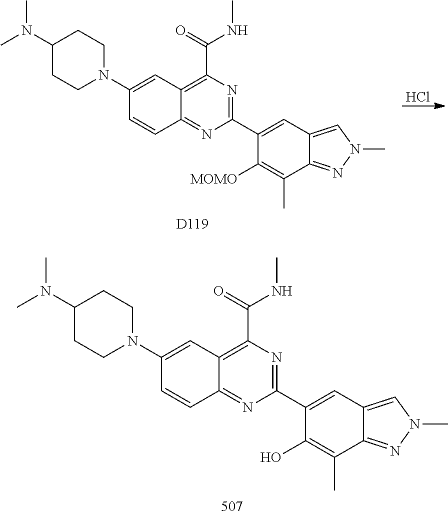

To a stirred solution of Intermediate D119 (15 mg, 0.029 mmol, 1 equiv) in dioxane (0.5 mL) was added HCl (gas) in 1,4-dioxane (0.5 mL) at room temperature. The resulting mixture was stirred for 1 h at room temperature and subsequently concentrated under reduced pressure. The crude product was purified by Prep-HPLC following Condition 13, Gradient 2 as previously described to afford 6-[4-(dimethylamino)piperidin-1-yl]-2-(6-hydroxy-2,7-dimethylindazol-5-yl)-N-methylquinazoline-4-carboxamide (Compound 507, 6 mg). LCMS: (ES, m/z): 420 [M+H]⁺. ¹H NMR: (400 MHz, Chloroform-d) δ 13.55 (s, 1H), 8.84 (s, 1H), 8.81 (d, J=2.8 Hz, 1H), 8.25 (d, J=5.6 Hz, 1H), 7.99 (s, 1H), 7.90 (d, J=9.3 Hz, 1H), 7.73 (dd, J=9.4, 2.8 Hz, 1H), 4.26 (s, 3H), 4.07 (d, J=12.7 Hz, 2H), 3.20 (d, J=5.1 Hz, 3H), 2.99 (t, J=11.8 Hz, 2H), 2.63 (d, J=4.3 Hz, 4H), 2.56 (s, 6H), 2.18 (s, 2H), 1.84 (d, J=12.5 Hz, 2H), 1.28 (s, 2H), 1.07 (s, 1H), 0.86 (s, 3H).

### Example 229: Synthesis of Compound 508

### Synthesis of Intermediate D121

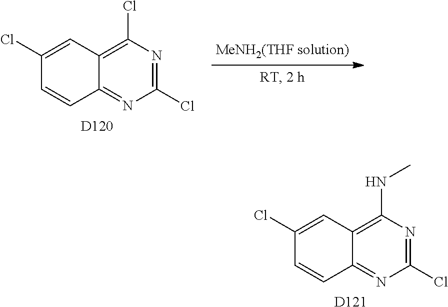

2,4,6-trichloroquinazoline (D120, 210 mg, 0.899 mmol, 1 equiv) was added to a stirred solution of 2M methylamine in THF (8 mL) at room temperature. The resulting mixture was stirred for 2 h at room temperature and then concentrated under reduced pressure. The residue was purified by silica gel column chromatography and eluted with PE/EA (5:1) to afford 2,6-dichloro-N-methylquinazolin-4-amine (D121, 200 mg). LCMS: (ES, m/z): 228 [M+H]⁺.

### Synthesis of Intermediate D122

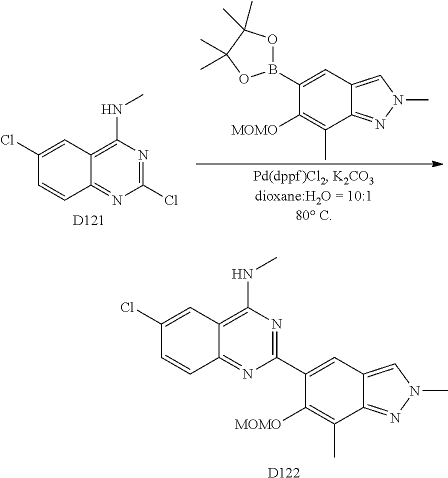

To a stirred mixture of Intermediate D121 (150 mg, 0.658 mmol, 1 equiv) and 6-(methoxymethoxy)-2,7-dimethyl-5-(4,4,5,5-tetramethyl-1,3,2-dioxaborolan-2-yl) indazole (327.72 mg, 0.987 mmol, 1.5 equiv) in dioxane/H₂O (10:1; 3.3 mL) were added K₂CO₃ (272.68 mg, 1.974 mmol, 3 equiv) and Pd(dppf)Cl₂ (481.22 mg, 0.658 mmol, 0.1 equiv) at room temperature under nitrogen atmosphere. The resulting mixture was stirred for 4 h at 80° C. under nitrogen atmosphere and later concentrated under reduced pressure. The residue was purified by silica gel column chromatography and eluted with PE/EA (1:1) to afford 6-chloro-2-[6-(methoxymethoxy)-2,7-dimethylindazol-5-yl]-N-methylquinazolin-4-amine (D122, 130 mg). LCMS: (ES, m/z): 398 [M+H]⁺.

### Synthesis of Intermediate D123

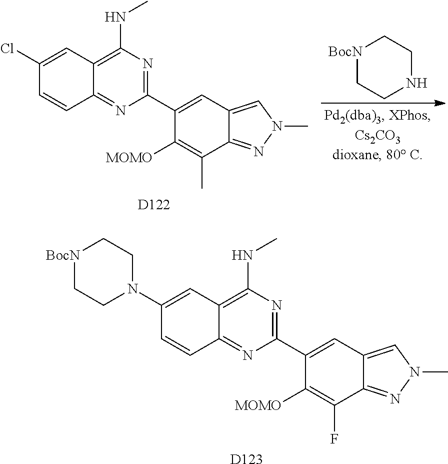

To a stirred mixture of Intermediate D122 (130 mg, 0.327 mmol, 1 equiv) and tert-butyl piperazine-1-carboxylate (79.12 mg, 0.425 mmol, 1.3 equiv) in dioxane (2.6 mL) were added Cs₂CO₃ (319.38 mg, 0.981 mmol, 3 equiv), X-Phos (31.15 mg, 0.065 mmol, 0.2 equiv) and Pd₂(dba)₃ (29.92 mg, 0.033 mmol, 0.1 equiv) at room temperature under nitrogen atmosphere. The resulting mixture was stirred for 4 h at 80° C. under nitrogen atmosphere and then concentrated under reduced pressure. The residue was purified by silica gel column chromatography and eluted with DCM/MeOH (20:1) to afford tert-butyl 4-{2-[6-(methoxymethoxy)-2,7-dimethylindazol-5-yl]-4-(methylamino)quinazolin-6-yl}piperazine-1-carboxylate (D123, 102 mg). LCMS: (ES, m/z): 578 [M+H]⁺.

### Synthesis of Compound 508

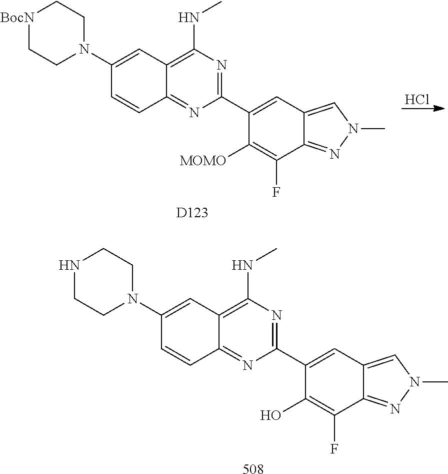

To a stirred solution of Intermediate D123 (102 mg, 0.186 mmol, 1 equiv) in dioxane (1 mL) was added HCl (gas) in 1,4-dioxane (1 mL) at room temperature. The resulting mixture was stirred for 1 h at room temperature and subsequently concentrated under reduced pressure. The crude product was purified by Prep-HPLC following Condition 13, Gradient 5 as previously described to afford 2,7-dimethyl-5-[4-(methylamino)-6-(piperazin-1-yl) quinazolin-2-yl]indazol-6-ol (Compound 508, 17.9 mg). LCMS: (ES, m/z): 404 [M+H]⁺. ¹H NMR: (400 MHz, DMSO-d₆) δ 14.74 (s, 1H), 8.83 (s, 1H), 8.41 (d, J=4.6 Hz, 1H), 8.35 (s, 1H), 7.63 (d, J=9.1 Hz, 1H), 7.59 (dd, J=9.2, 2.4 Hz, 1H), 7.48 (d, J=2.4 Hz, 1H), 4.13 (s, 3H), 3.25-3.16 (m, 7H), 2.89 (t, J=5.0 Hz, 4H), 2.36 (s, 3H).

### Example 230: Synthesis of Compound 509

### Synthesis of Intermediate D124

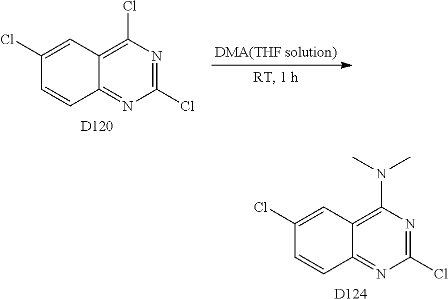

Intermediate D120 (210 mg, 0.899 mmol, 1 equiv) was added to a stirred solution of 2 M dimethylamine in THF (8 mL) at room temperature. The resulting mixture was stirred for 2 h at room temperature and then concentrated under reduced pressure. The residue was purified by silica gel column chromatography and eluted with PE/EA (5:1) to afford 2,6-dichloro-N,N-dimethylquinazolin-4-amine (D124, 200 mg). LCMS: (ES, m/z): 242 [M+H]⁺.

### Synthesis of Intermediate D125

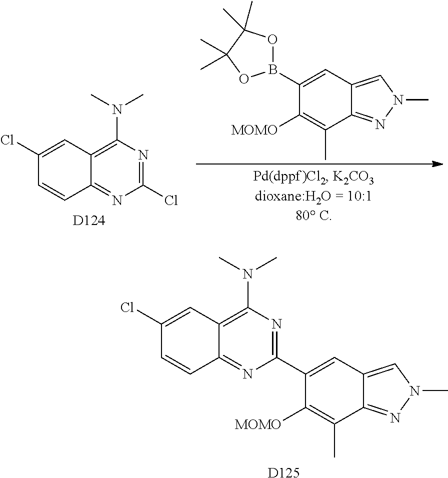

To a stirred mixture of Intermediate D124 (200 mg, 0.826 mmol, 1 equiv) and 6-(methoxymethoxy)-2,7-dimethyl-5-(4,4,5,5-tetramethyl-1,3,2-dioxaborolan-2-yl) indazole (301.88 mg, 0.909 mmol, 1.1 equiv) in dioxane/H₂O (10:1, 2.2 mL) were added K₂CO₃ (342.52 mg, 2.478 mmol, 3 equiv) and Pd(dppf)Cl₂ (60.45 mg, 0.083 mmol, 0.1 equiv) at room temperature under nitrogen atmosphere. The resulting mixture was stirred for 4 h at 80° C. (under nitrogen atmosphere) and subsequently concentrated under reduced pressure. The residue was purified by silica gel column chromatography and eluted with PE/EA (1:1) to afford 6-chloro-2-[6-(methoxymethoxy)-2,7-dimethylindazol-5-yl]-N, N-dimethylquinazolin-4-amine (D125, 130 mg). LCMS: (ES, m/z): 412 [M+H]⁺.

### Synthesis of Intermediate D126

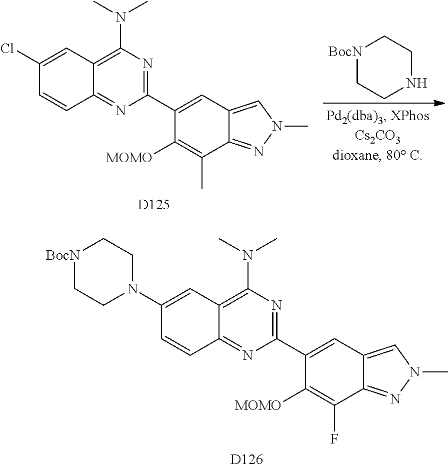

To a stirred mixture of Intermediate D125 (130 mg, 0.316 mmol, 1 equiv) and tert-butyl piperazine-1-carboxylate (88.18 mg, 0.474 mmol, 1.5 equiv) in dioxane (2.6 mL) were added Cs₂CO₃ (309.46 mg, 0.948 mmol, 3 equiv), X-Phos (30.09 mg, 0.063 mmol, 0.2 equiv) and Pd₂(dba)₃ (28.90 mg, 0.032 mmol, 0.1 equiv) at room temperature under nitrogen atmosphere. The resulting mixture was stirred for 4 h at 80° C. under nitrogen atmosphere and subsequently concentrated under reduced pressure. The residue was purified by silica gel column chromatography and eluted with DCM/MeOH (20:1) to afford tert-butyl 4-[4-(dimethylamino)-2-[6-(methoxymethoxy)-2,7-dimethylindazol-5-yl]quinazolin-6-yl]piperazine-1-carboxylate (D126, 110 mg). LCMS: (ES, m/z): 562 [M+H]⁺.

### Synthesis of Compound 509

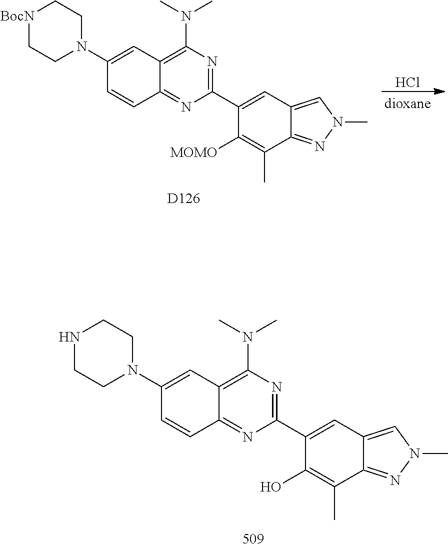

To a stirred solution of Intermediate D126 (110 mg, 0.196 mmol, 1 equiv) in dioxane (1 mL) was added HCl (gas) in 1,4-dioxane (1 mL) at room temperature. The resulting mixture was stirred for 1 h at room temperature and then concentrated under reduced pressure. The crude product was purified by Prep-HPLC following Condition 13, Gradient 2 as previously delineated to afford 5-[4-(dimethylamino)-6-(piperazin-1-yl)quinazolin-2-yl]-2,7-dimethylindazol-6-ol (Compound 509, 14 mg). LCMS: (ES, m/z): 418 [M+H]⁺. ¹H NMR: (400 MHz, DMSO-d6) δ 14.45 (s, 1H), 8.78 (s, 1H), 8.35 (s, 1H), 7.71 (d, J=9.2 Hz, 1H), 7.65 (dd, J=9.2, 2.5 Hz, 1H), 7.32 (d, J=2.5 Hz, 1H), 4.13 (s, 3H), 3.43 (s, 6H), 3.19 (t, J=5.0 Hz, 4H), 2.89 (t, J=5.0 Hz, 4H), 2.37 (s, 3H).

### Example 231: Synthesis of Compound 510

### Synthesis of Intermediate D127

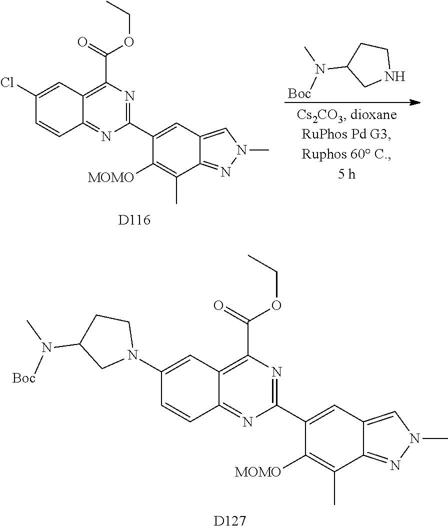

To a stirred mixture of Intermediate D116 (160 mg, 0.363 mmol, 1 equiv) and tert-butyl N-methyl-N-(pyrrolidin-3-yl)carbamate (94.49 mg, 0.472 mmol, 1.3 equiv) in dioxane (3 mL) were added Cs₂CO₃ (354.73 mg, 1.089 mmol, 3 equiv), Ruphos (33.87 mg, 0.073 mmol, 0.2 equiv) and RuPhos Palladacycle Gen.3 (30.35 mg, 0.036 mmol, 0.1 equiv) at room temperature under nitrogen atmosphere. The resulting mixture was stirred for 5 h at 60° C. under nitrogen atmosphere and subsequently concentrated under reduced pressure. The residue was purified by silica gel column chromatography and eluted with DCM/MeOH (20:1) to afford ethyl 6-{3-[(tert-butoxycarbonyl)(methyl)amino]pyrrolidin-1-yl}-2-[6-(methoxymethoxy)-2,7-dimethylindazol-5-yl]quinazoline-4-carboxylate (D127, 150 mg). LCMS: (ES, m/z): 605 [M+H]

### Synthesis of Intermediate D128

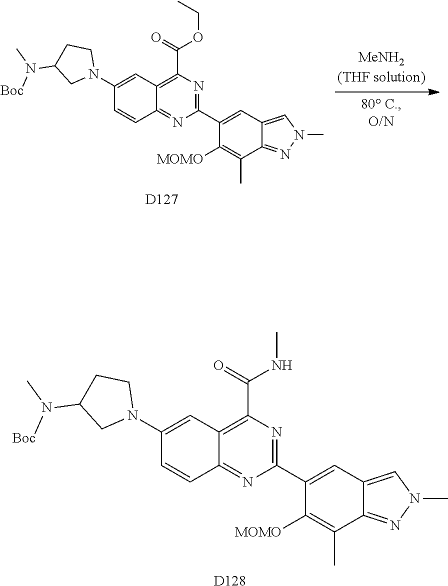

Intermediate D127 (150 mg, 0.248 mmol, 1 equiv) was added to a stirred solution of 2 M methylamine in THE (8 mL) at room temperature. The resulting mixture was stirred overnight at 80° C. and then concentrated under reduced pressure to afford tert-butyl N-(1-{2-[6-(methoxymethoxy)-2,7-dimethylindazol-5-yl]-4-(methylcarbamoyl)quinazolin-6-yl}pyrrolidin-3-yl)-N-methylcarbamate (D128, 112 mg). LCMS: (ES, m/z): 590 [M+H]⁺.

### Synthesis of Compound 510

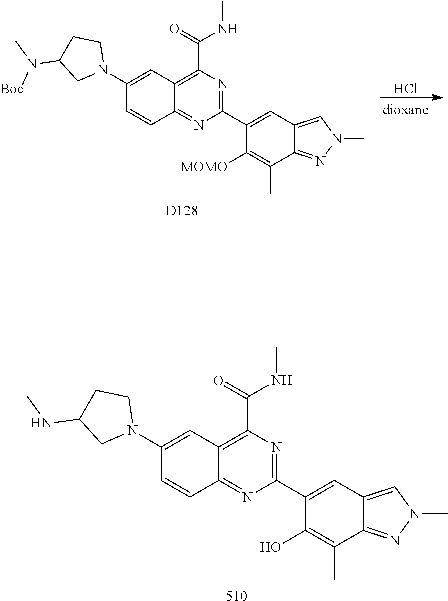

To a stirred solution of Intermediate D128 (75 mg, 0.127 mmol, 1 equiv) in dioxane (1 mL) was added HCl (gas) in 1,4-dioxane (1 mL) at room temperature. The resulting mixture was stirred for 1 h at room temperature and subsequently concentrated under reduced pressure. The crude product was purified by Prep-HPLC following Condition 13, Gradient 3 as previously described to afford 2-(6-hydroxy-2,7-dimethylindazol-5-yl)-N-methyl-6-[3-(methylamino)pyrrolidin-1-yl]quinazoline-4-carboxamide (Compound 510, 13.7 mg). LCMS: (ES, m/z): 446 [M+H]⁺. ¹H NMR: (400 MHz, DMSO-d₆) δ 13.47 (s, 1H), 9.14 (d, J=5.4 Hz, 1H), 9.04 (s, 1H), 8.39 (s, 1H), 7.97 (d, J=9.2 Hz, 1H), 7.72 (d, J=2.8 Hz, 1H), 7.58 (dd, J=9.1, 2.7 Hz, 1H), 4.16 (s, 3H), 3.57 (dd, J=9.9, 6.0 Hz, 1H), 3.49 (t, J=7.3 Hz, 1H), 3.42 (d, J=7.4 Hz, 1H), 3.18 (dd, J=10.0, 4.4 Hz, 1H), 2.96 (d, J=4.5 Hz, 3H), 2.40 (s, 3H), 2.34 (s, 3H), 2.21-2.11 (m, 1H), 1.97 (s, 1H), 1.89 (dd, J=12.4, 6.4 Hz, 1H).

### Example 232: Synthesis of Compound 511

### Synthesis of Intermediate D129

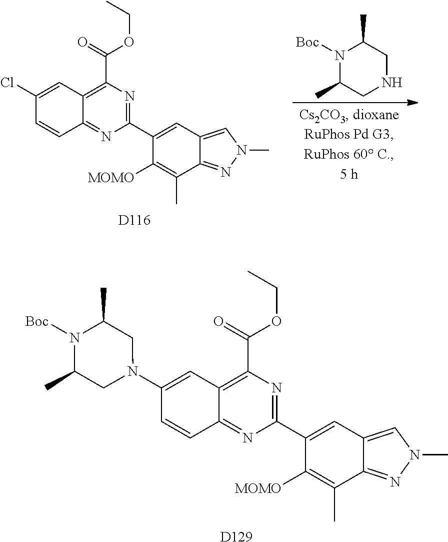

To a stirred mixture of Intermediate D116 (160 mg, 0.363 mmol, 1 equiv) and tert-butyl (2R,6S)-2,6-dimethylpiperazine-1-carboxylate (116.66 mg, 0.544 mmol, 1.5 equiv) in dioxane (3 mL) were added Cs₂CO₃ (354.73 mg, 1.089 mmol, 3 equiv), RuPhos (33.87 mg, 0.073 mmol, 0.2 equiv) and RuPhos Palladacycle Gen.3 (30.35 mg, 0.036 mmol, 0.1 equiv) at room temperature under nitrogen atmosphere. The resulting mixture was stirred for 5 h at 60° C. under nitrogen atmosphere and later concentrated under reduced pressure. The residue was purified by silica gel column chromatography and eluted with DCM/MeOH (20:1) to afford ethyl 6-[(3R,5S)-4-(tert-butoxycarbonyl)-3,5-dimethylpiperazin-1-yl]-2-[6-(methoxymethoxy)-2,7-dimethylindazol-5-yl]quinazoline-4-carboxylate (D129, 150 mg). LCMS: (ES, m/z): 619 [M+H]⁺.

### Synthesis of Intermediate D130

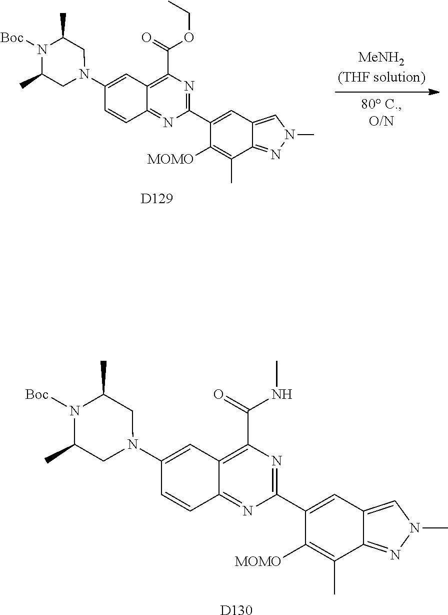

Intermediate D129 (150 mg, 0.242 mmol, 1 equiv) was added to 2 M methylamine in THE (8 mL) at room temperature. The resulting mixture was stirred overnight at 80° C. and then concentrated under reduced pressure to afford tert-butyl (2R,6S)-4-{2-[6-(methoxymethoxy)-2,7-dimethylindazol-5-yl]-4-(methylcarbamoyl)quinazolin-6-yl}-2,6-dimethylpiperazine-1-carboxylate (D130, 124 mg). LCMS: (ES, m/z): 604 [M+H]⁺.

### Synthesis of Compound 511

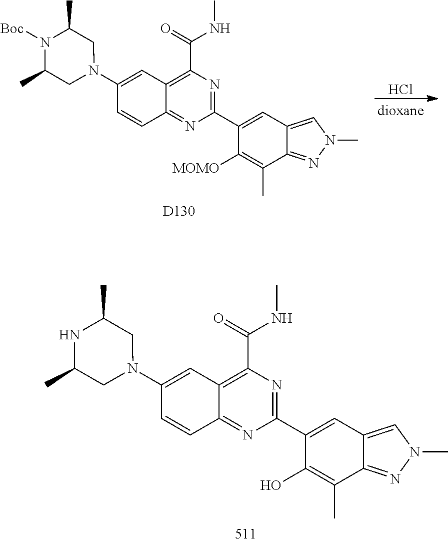

To a stirred solution of Intermediate D130 (75 mg, 0.124 mmol, 1 equiv) in dioxane (1 mL) was added HCl (gas) in 1,4-dioxane (1 mL) at room temperature. The resulting mixture was stirred for 1 h at room temperature and subsequently concentrated under reduced pressure. The crude product was purified by Prep-HPLC following Condition 13, Gradient 3 as previously delineated to afford 6-[(3R,5S)-3,5-dimethylpiperazin-1-yl]-2-(6-hydroxy-2,7-dimethylindazol-5-yl)-N-methylquinazoline-4-carboxamide (Compound 511, 16.9 mg). Absolute stereochemistry of the compound was arbitrarily assigned. LCMS: (ES, m/z): 460 [M+H]⁺. ¹H NMR: (400 MHz, DMSO-d₆) δ 13.40 (s, 1H), 9.20 (d, J=5.0 Hz, 1H), 9.09 (s, 1H), 8.42 (s, 1H), 8.14 (d, J=2.2 Hz, 1H), 8.00 (d, J=2.9 Hz, 2H), 4.16 (s, 3H), 3.76 (d, J=10.7 Hz, 2H), 2.97 (d, J=4.8 Hz, 3H), 2.90 (s, 2H), 2.41 (s, 3H), 2.32 (t, J=11.1 Hz, 2H), 1.09 (d, J=6.2 Hz, 6H).

### Example 233: Synthesis of Compound 512

### Synthesis of Intermediate D131

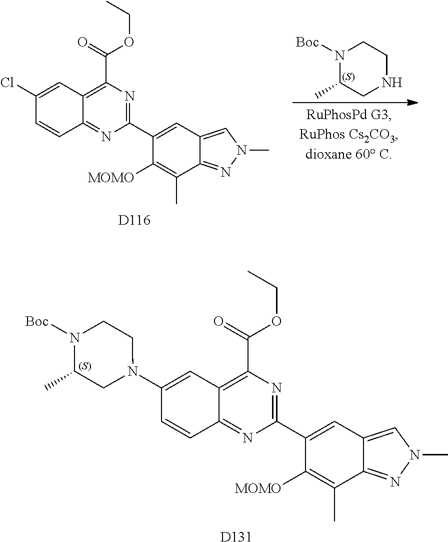

To a stirred mixture of Intermediate D116 (150 mg, 0.340 mmol, 1 equiv) and tert-butyl (2S)-2-methylpiperazine-1-carboxylate (88.58 mg, 0.442 mmol, 1.3 equiv) in dioxane (3 mL) were added Cs₂CO₃ (332.56 mg, 1.020 mmol, 3 equiv), RuPhos (31.75 mg, 0.068 mmol, 0.2 equiv) and RuPhos Palladacycle Gen.3 (28.46 mg, 0.034 mmol, 0.1 equiv) at room temperature under nitrogen atmosphere. The resulting mixture was stirred for 5 h at 60° C. under nitrogen atmosphere and later concentrated under reduced pressure. The residue was purified by silica gel column chromatography and eluted with DCM/MeOH (20:1) to afford ethyl 6-[(3S)-4-(tert-butoxycarbonyl)-3-methylpiperazin-1-yl]-2-[6-(methoxymethoxy)-2,7-dimethylindazol-5-yl]quinazoline-4-carboxylate (D131, 150 mg). LCMS: (ES, m/z): 605 [M+H]⁺.

### Synthesis of Intermediate D132

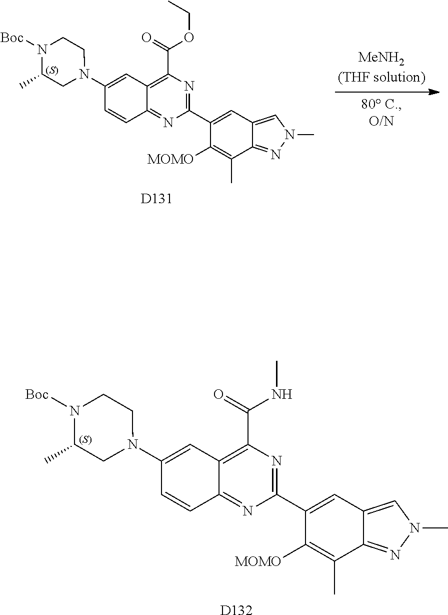

Intermediate D131 (150 mg, 0.248 mmol, 1 equiv) was added to a stirred solution of 2M methylamine in THE (8 mL) at room temperature. The resulting mixture was stirred overnight at 80° C. and then concentrated under reduced pressure to afford tert-butyl (2S)-4-{2-[6-(methoxymethoxy)-2,7-dimethylindazol-5-yl]-4-(methylcarbamoyl) quinazolin-6-yl}-2-methylpiperazine-1-carboxylate (D132, 121 mg). LCMS: (ES, m/z): 560 [M+H]⁺.

### Synthesis of Compound 512

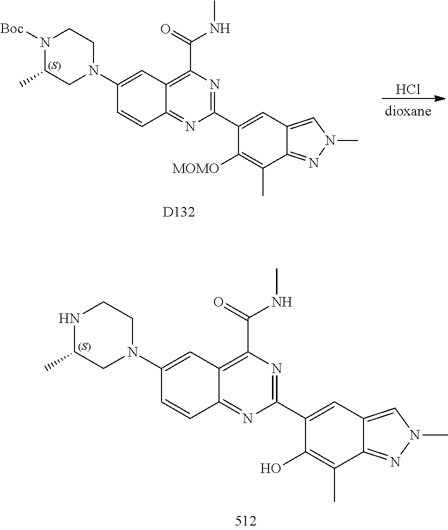

To a stirred solution of Intermediate D132 (75 mg, 0.127 mmol, 1 equiv) in dioxane (1 mL) was added HCl (gas) in 1,4-dioxane (1 mL) at room temperature. The resulting mixture was stirred for 1 h at room temperature and then concentrated under reduced pressure. The crude product was purified by Prep-HPLC following Condition 13, Gradient 4 as previously described to afford 2-(6-hydroxy-2,7-dimethylindazol-5-yl)-N-methyl-6-[(3S)-3-methylpiperazin-1-yl]quinazoline-4-carboxamide (Compound 512, 19.5 mg). Absolute stereochemistry of the compound was arbitrarily assigned. LCMS: (ES, m/z): 446 [M+H]⁺. ¹H NMR: (400 MHz, DMSO-d₆) δ 13.40 (s, 1H), 9.19 (d, J=4.7 Hz, 1H), 9.09 (s, 1H), 8.42 (s, 1H), 8.13 (d, J=1.7 Hz, 1H), 7.99 (d, J=1.6 Hz, 2H), 4.16 (s, 3H), 3.74 (t, J=10.3 Hz, 2H), 3.04 (d, J=11.3 Hz, 1H), 2.97 (d, J=4.8 Hz, 3H), 2.83 (s, 2H), 2.82-2.70 (m, 1H), 2.41 (s, 4H), 1.08 (d, J=6.3 Hz, 3H).

### Example 234: Synthesis of Compound 514

### Synthesis of Intermediate D134

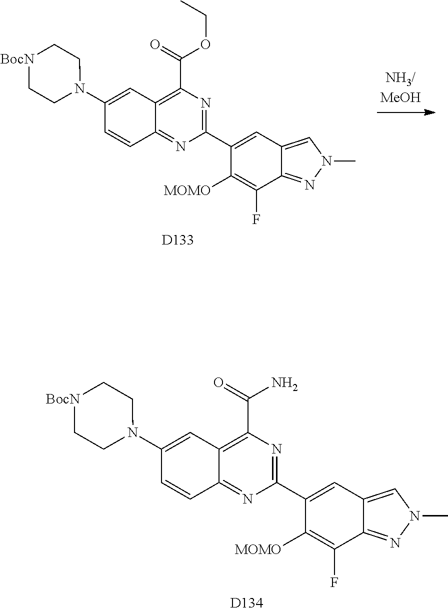

Ethyl 6-[4-(tert-butoxycarbonyl) piperazin-1-yl]-2-[7-fluoro-6-(methoxymethoxy)-2-methylindazol-5-yl]quinazoline-4-carboxylate (D133, 240 mg, 0.404 mmol, 1 equiv) was added to stirred solution of NH₃(g) in MeOH (8 mL) at room temperature. The resulting mixture was stirred overnight at 80° C. and subsequently concentrated under reduced pressure to afford tert-butyl 4-{4-carbamoyl-2-[7-fluoro-6-(methoxymethoxy)-2-methylindazol-5-yl]quinazolin-6-yl}piperazine-1-carboxylate (D134, 200 mg). LCMS: (ES, m/z): 566 [M+H]⁺.

### Synthesis of Intermediate D135

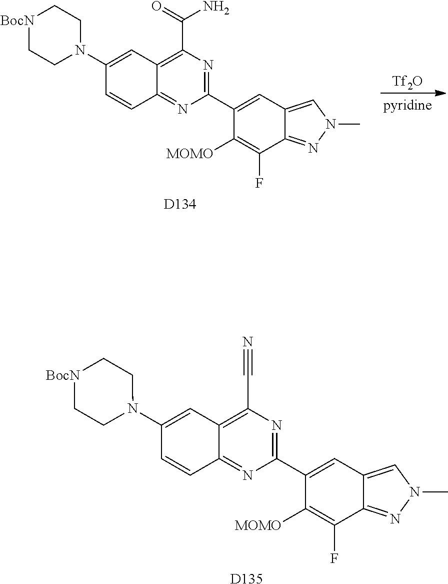

To a stirred solution of Intermediate D134 (200 mg, 0.354 mmol, 1 equiv) in DCM (2 mL) and pyridine (0.2 mL) was added trifluoroacetic anhydride (148.54 mg, 0.708 mmol, 2 equiv) dropwise at 0° C. The resulting mixture was stirred for 1 h at room temperature and then concentrated under reduced pressure. The residue was purified by silica gel column chromatography and eluted with PE/EA (5:1) to afford tert-butyl 4-{4-cyano-2-[7-fluoro-6-(methoxymethoxy)-2-methylindazol-5-yl]quinazolin-6-yl}piperazine-1-carboxylate (D135, 127 mg). LCMS: (ES, m/z): 548 [M+H]⁺.

### Synthesis of Compound 514

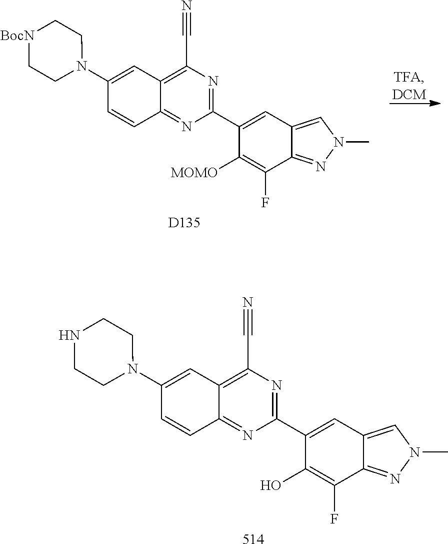

To a stirred solution of Intermediate D135 (127 mg, 0.232 mmol, 1 equiv) in DCM (1 mL) was added TFA (1 mL) at room temperature. The resulting mixture was stirred for 1 h at room temperature and then concentrated under reduced pressure. The crude product was purified by Prep-HPLC following Condition 7, Gradient 4 as previously described to afford 2-(7-fluoro-6-hydroxy-2-methylindazol-5-yl)-6-(piperazin-1-yl) quinazoline-4-carbonitrile; trifluoroacetic acid (Compound 514, 61.2 mg). LCMS: (ES, m/z): 404 [M+H]⁺. ¹H NMR: (400 MHz, Methanol-d₄) δ 8.31-8.23 (m, 2H), 7.92 (dd, J=9.5, 2.6 Hz, 1H), 7.76 (d, J=9.3 Hz, 1H), 6.73 (d, J=2.7 Hz, 1H), 4.21 (s, 3H), 3.75-3.68 (m, 4H), 3.52 (t, J=5.2 Hz, 4H).

### Example 235: Synthesis of Compound 518

### Synthesis of Intermediate D136

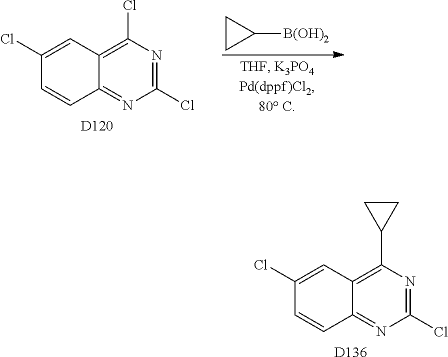

Into a 40 mL vial were added Intermediate D120 (1.0 g, 4.283 mmol, 1.0 equiv), cyclopropylboronic acid (0.37 g, 4.283 mmol, 1.0 equiv), THF (20 mL), Pd(dppf)Cl₂ (0.35 g, 0.428 mmol, 0.1 equiv) and K₃PO₄ (2.73 g, 12.849 mmol, 3.0 equiv) at room temperature. The resulting mixture was stirred overnight at 80° C. under nitrogen atmosphere and subsequently concentrated under reduced pressure. The residue was purified by silica gel column chromatography and eluted with PE/EA (5:1) to afford 2,6-dichloro-4-cyclopropylquinazoline (D136, 0.4 g). LCMS: (ES, m/z): 239 [M+H]⁺.

### Synthesis of Intermediate D137

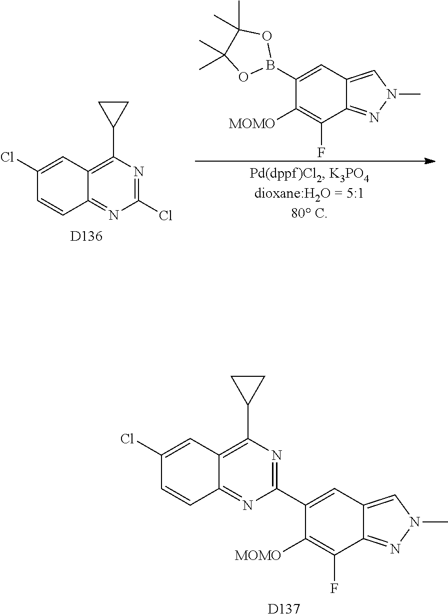

Into an 8 mL vial were added Intermediate D136 (200 mg, 0.836 mmol, 1.0 equiv), 7-fluoro-6-(methoxymethoxy)-2-methyl-5-(4,4,5,5-tetramethyl-1,3,2-dioxaborolan-2-yl) indazole (281.20 mg, 0.836 mmol, 1.0 equiv), dioxane (2 ml), water (0.4 mL), Pd(dppf)Cl₂ (68.14 mg, 0.084 mmol, 0.1 equiv) and K₃PO₄ (532.66 mg, 2.508 mmol, 3.0 equiv) at room temperature. The resulting mixture was stirred overnight at 80° C. under nitrogen atmosphere and then concentrated under reduced pressure. The residue was purified by silica gel column chromatography and eluted with PE/EA (3:1) to afford 6-chloro-4-cyclopropyl-2-[7-fluoro-6-(methoxymethoxy)-2-methylindazol-5-yl]quinazoline (D137, 230 mg). LCMS: (ES, m/z): 413 [M+H]⁺.

### Synthesis of Intermediate D138

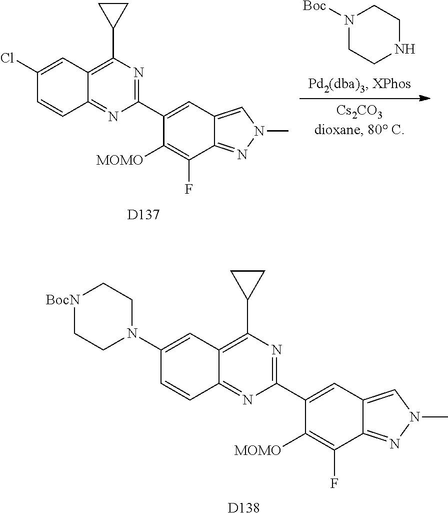

Into an 8 mL vial were added Intermediate D137 (200 mg, 0.484 mmol, 1.0 equiv), tert-butyl piperazine-1-carboxylate (135.34 mg, 0.726 mmol, 1.5 equiv), dioxane (4 mL), XPhos (46.19 mg, 0.097 mmol, 0.2 equiv), Pd₂(dba)₃ (44.36 mg, 0.048 mmol, 0.1 equiv) and Cs₂CO₃ (236.76 mg, 0.726 mmol, 1.5 equiv) at room temperature. The resulting mixture was stirred overnight at 80° C. under nitrogen atmosphere and subsequently concentrated under reduced pressure. The residue was purified by silica gel column chromatography and eluted with CH₂Cl₂/MeOH (20:1) to afford tert-butyl 4-{4-cyclopropyl-2-[7-fluoro-6-(methoxymethoxy)-2-methylindazol-5-yl]quinazolin-6-yl}piperazine-1-carboxylate (D138, 160 mg). LCMS: (ES, m/z): 563 [M+H]⁺.

### Synthesis of Compound 518

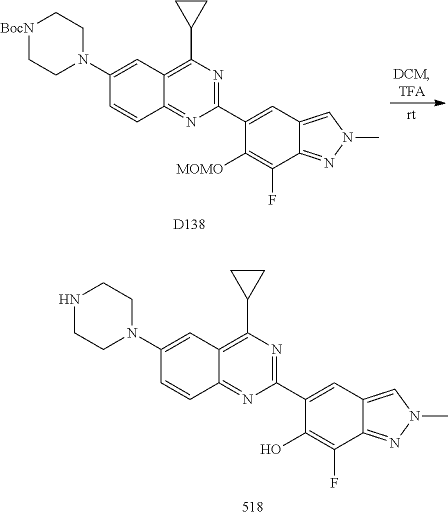

Into an 8 mL vial were added Intermediate D138 (80 mg, 0.142 mmol, 1 equiv), DCM (1 mL) and TFA (1 mL) at room temperature. The resulting mixture was stirred for 2 h at room temperature and then concentrated under reduced pressure. The residue was purified by reverse flash chromatography following Condition 4, Gradient 4 as previously delineated to afford 5-[4-cyclopropyl-6-(piperazin-1-yl)quinazolin-2-yl]-7-fluoro-2-methylindazol-6-ol; trifluoroacetic acid (Compound 518, 22 mg). LCMS: (ES, m/z): 418 [M+H]⁺. ¹H NMR: (400 MHz, DMSO-d₆) δ 13.85 (s, 1H), 8.87 (s, 2H), 8.77 (s, 1H), 8.55 (d, J=2.6 Hz, 1H), 7.99 (d, J=9.3 Hz, 1H), 7.93 (dd, J=9.3, 2.5 Hz, 1H), 7.79 (d, J=2.5 Hz, 1H), 4.17 (s, 3H), 3.65 (t, J=5.1 Hz, 4H), 3.33 (d, J=6.0 Hz, 4H), 3.22 (tt, J=8.1, 4.5 Hz, 1H), 1.45 (p, J=3.9, 3.5 Hz, 2H), 1.35 (dt, J=8.1, 3.3 Hz, 2H).

### Example 236: Synthesis of Compound 519

### Synthesis of Intermediate D139

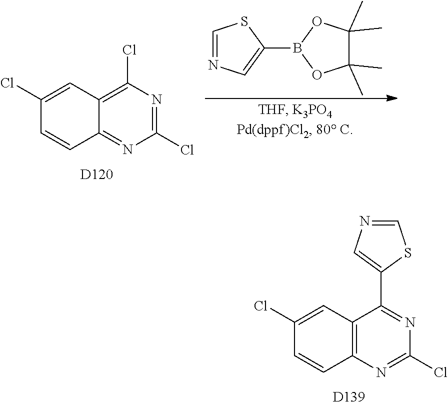

To a stirred mixture of Intermediate D120 (310 mg, 1.328 mmol, 1 equiv) and 5-(4,4,5,5-tetramethyl-1,3,2-dioxaborolan-2-yl)-1,3-thiazole (308.30 mg, 1.461 mmol, 1.1 equiv) in THF (6 mL) were added K₃PO₄ (845.50 mg, 3.984 mmol, 3 equiv) and Pd(dppf)Cl₂ (97.15 mg, 0.133 mmol, 0.1 equiv) at room temperature under nitrogen atmosphere. The resulting mixture was stirred for overnight at 80° C. under nitrogen atmosphere and then concentrated under reduced pressure. The residue was purified by silica gel column chromatography and eluted with PE/EA (1:1) to afford 2,6-dichloro-4-(1,3-thiazol-5-yl) quinazoline (D139, 300 mg). LCMS: (ES, m/z): 282 [M+H]⁺.

### Synthesis of Intermediate D140

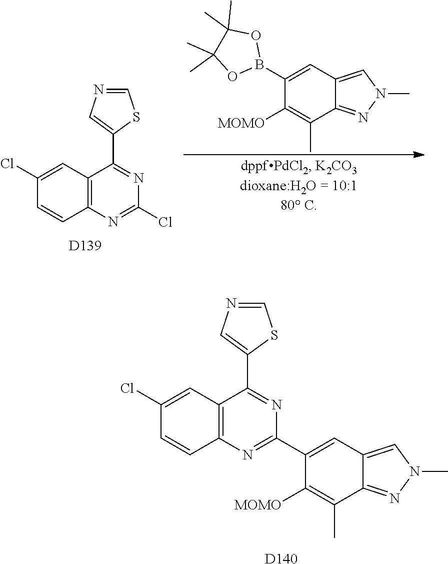

To a stirred mixture of Intermediate D139 (300 mg, 1.063 mmol, 1 equiv) and 6-(methoxymethoxy)-2,7-dimethyl-5-(4,4,5,5-tetramethyl-1,3,2-dioxaborolan-2-yl) indazole (388.56 mg, 1.169 mmol, 1.1 equiv) in dioxane/H₂O (10:1; 6.6 mL) were added K₂CO₃ (440.86 mg, 3.189 mmol, 3 equiv) and Pd(dppf)Cl₂ (77.80 mg, 0.106 mmol, 0.1 equiv) at room temperature under nitrogen atmosphere. The resulting mixture was stirred overnight at 80° C. under nitrogen atmosphere and subsequently concentrated under reduced pressure. The residue was purified by silica gel column chromatography and eluted with PE/EA (1:1) to afford 6-chloro-2-[6-(methoxymethoxy)-2,7-dimethylindazol-5-yl]-4-(1,3-thiazol-5-yl) quinazoline (D140, 210 mg). LCMS: (ES, m/z): 452 [M+H]⁺.

### Synthesis of Intermediate D141

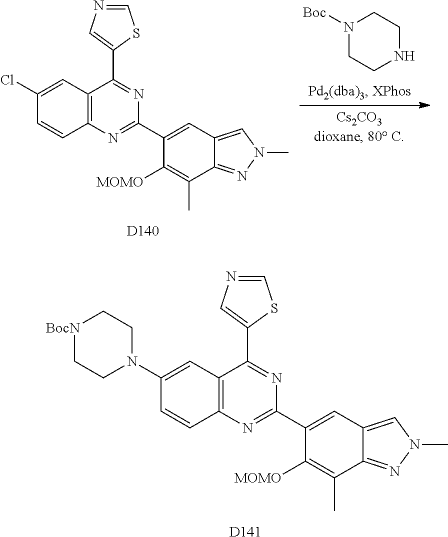

To a stirred mixture of Intermediate D140 (210 mg, 0.465 mmol, 1 equiv) and tert-butyl piperazine-1-carboxylate (129.82 mg, 0.698 mmol, 1.5 equiv) in dioxane (3 mL) were added Cs₂CO₃ (455.60 mg, 1.395 mmol, 3 equiv), X-Phos (44.30 mg, 0.093 mmol, 0.2 equiv) and Pd₂(dba)₃ (42.55 mg, 0.047 mmol, 0.1 equiv) at room temperature under nitrogen atmosphere. The resulting mixture was stirred overnight at 80° C. under nitrogen atmosphere and later concentrated under reduced pressure. The residue was purified by silica gel column chromatography and eluted with DCM/MeOH (20:1) to afford tert-butyl 4-{2-[6-(methoxymethoxy)-2,7-dimethylindazol-5-yl]-4-(1,3-thiazol-5-yl) quinazolin-6-yl}piperazine-1-carboxylate (D141, 120 mg). LCMS: (ES, m/z): 602 [M+H]⁺.

### Synthesis of Compound 519

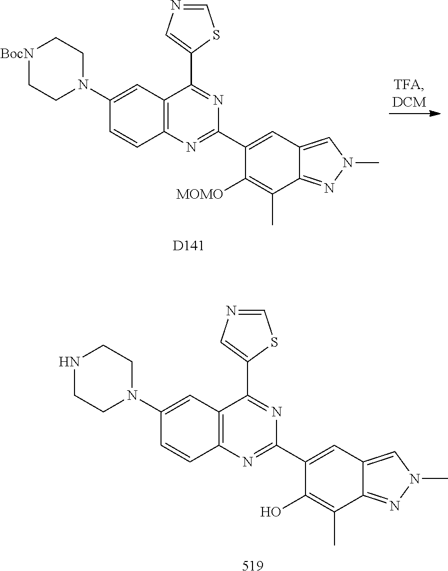

To a stirred solution of Intermediate D141 (120 mg, 0.199 mmol, 1 equiv) in DCM (1 mL) was added TFA (1 mL) at room temperature. The resulting mixture was stirred for 1 h at room temperature and subsequently concentrated under reduced pressure. The crude product was purified by Prep-HPLC following Condition 13, Gradient 3 as previously described to afford 2,7-dimethyl-5-[6-(piperazin-1-yl)-4-(1,3-thiazol-5-yl) quinazolin-2-yl]indazol-6-ol (Compound 519, 29.4 mg). LCMS: (ES, m/z): 458 [M+H]⁺. ¹H NMR: (400 MHz, DMSO-d₆) δ 13.45 (s, 1H), 9.48 (s, 1H), 8.96 (s, 1H), 8.87 (s, 1H), 8.44 (s, 1H), 8.02 (d, J=2.8 Hz, 2H), 7.55 (d, J=1.9 Hz, 1H), 4.15 (s, 3H), 3.36 (t, J=5.0 Hz, 4H), 2.94 (t, J=5.1 Hz, 4H), 2.41 (s, 3H).

### Example 237: Synthesis of Compound 520

### Synthesis of Intermediate D143

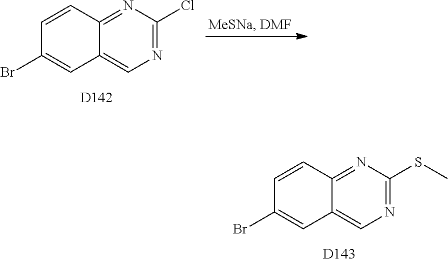

A solution of 6-bromo-2-chloroquinazoline (D142, 600 mg, 2.464 mmol, 1 equiv) and CH₃SNa (259 mg, 3.696 mmol, 1.5 equiv) in DMF (12 mL) was stirred for 3 h at room temperature, quenched by the addition of water (80 mL) at room temperature, and then extracted with EtOAc (3×10 mL). The combined organic layers were washed with brine (1×5 mL), dried over anhydrous Na₂SO₄, and the filtrate was concentrated under reduced pressure. The residue was purified by silica gel column chromatography and eluted with PE/EA (20:1) to afford 6-bromo-2-(methylsulfanyl)quinazoline (D143, 400 mg). LCMS: (ES, m/z): 255 [M+H]⁺.

### Synthesis of Intermediate D144

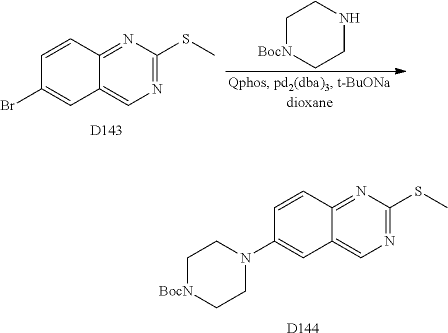

To a stirred solution of Intermediate D143 (300 mg, 1.176 mmol, 1 equiv) and tert-butyl piperazine-1-carboxylate (241 mg, 1.294 mmol, 1.1 equiv) in dioxane (10 mL) were added t-BuONa (226 mg, 2.352 mmol, 2 equiv), 1,2,3,4,5-pentaphenyl-1-(di-tert-butylphosphino)ferrocene (168 mg, 0.235 mmol, 0.2 equiv) and Pd₂(dba)₃ (108 mg, 0.118 mmol, 0.1 equiv) at room temperature under nitrogen atmosphere. The resulting mixture was stirred for 2 h at 90° C. under nitrogen atmosphere, subsequently allowed to cool to room temperature, and finally concentrated under reduced pressure. The residue was purified by silica gel column chromatography and eluted with PE/EA (4:1) to afford tert-butyl 4-[2-(methylsulfanyl)quinazolin-6-yl]piperazine-1-carboxylate (D144, 220 mg). LCMS: (ES, m/z): 361 [M+H]⁺.

### Synthesis of Intermediate D145

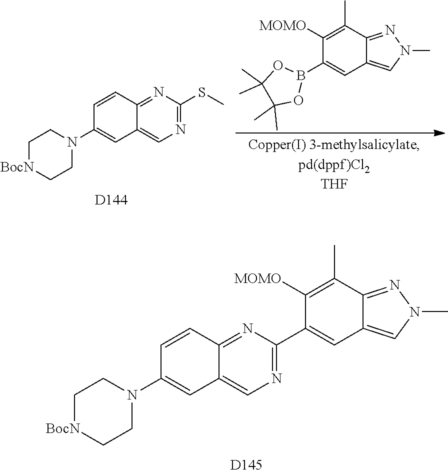

To a stirred solution of Intermediate D144 (220 mg, 0.610 mmol, 1 equiv) and 6-(methoxymethoxy)-2,7-dimethyl-5-(4,4,5,5-tetramethyl-1,3,2-dioxaborolan-2-yl) indazole (203 mg, 0.610 mmol, 1 equiv) in THE (8 mL) were added Pd(dppf)Cl₂·CH₂Cl₂ (50 mg, 0.061 mmol, 0.1 equiv) and Copper(I) 3-methylsalicylate (289 mg, 1.342 mmol, 2.2 equiv) at room temperature under nitrogen atmosphere. The resulting mixture was stirred for 2 h at 90° C. under nitrogen atmosphere, allowed to cool to room temperature, and then concentrated under reduced pressure. The residue was purified by reversed-phase flash chromatography following Condition 7, Gradient 5 as previously delineated to afford tert-butyl 4-{2-[6-(methoxymethoxy)-2,7-dimethylindazol-5-yl]quinazolin-6-yl}piperazine-1-carboxylate (D145, 125 mg). LCMS: (ES, m/z): 519 [M+H]⁺.

### Synthesis of Compound 520

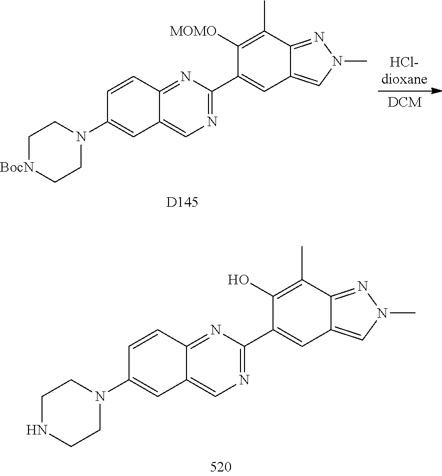

A solution of Intermediate D145 (125 mg, 0.241 mmol, 1 equiv) and 4 M HCl (gas) in 1,4-dioxane (2 mL) in DCM (8 mL) was stirred for 2 h at room temperature and then concentrated under reduced pressure. The residue was purified by reversed-phase flash chromatography following Condition 10, Gradient 14 as previously described to afford 2,7-dimethyl-5-[6-(piperazin-1-yl)quinazolin-2-yl]indazol-6-ol (Compound 520, 51 mg). LCMS: (ES, m/z): 375 [M+H]⁺. ¹H NMR: (300 MHz, Methanol-d₄) δ 9.42 (s, 1H), 8.96 (d, J=0.8 Hz, 1H), 8.24 (s, 1H), 7.89 (d, J=2.4 Hz, 2H), 7.34 (s, 1H), 4.20 (s, 3H), 3.43-3.33 (m, 4H), 3.10-3.01 (m, 4H), 2.49 (d, J=0.7 Hz, 3H), 1.31 (s, 1H).

### Example 238: Synthesis of Compound 521

### Synthesis of Intermediate D146

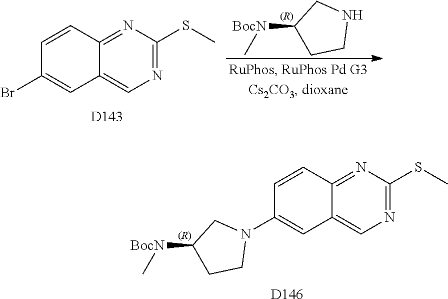

To a stirred solution of 6-bromo-2-(methylsulfanyl)quinazoline (D143, 260 mg, 1.019 mmol, 1 equiv) and tert-butyl N-methyl-N-[(3R)-pyrrolidin-3-yl]carbamate (307 mg, 1.528 mmol, 1.5 equiv) in dioxane (10 mL) were added t-BuONa (196 mg, 2.038 mmol, 2 equiv), 1,2,3,4,5-pentaphenyl-1-(di-tert-butylphosphino)ferrocene (145 mg, 0.204 mmol, 0.2 equiv) and Pd₂(dba)₃ (94 mg, 0.102 mmol, 0.1 equiv) at room temperature under nitrogen atmosphere. The resulting mixture was stirred for 4 h at 90° C. under nitrogen atmosphere, allowed to cool to room temperature, quenched by the addition of water (50 mL) at room temperature, and subsequently extracted with EtOAc (3×8 mL). The combined organic layers were washed with brine (1×5 mL), dried over anhydrous Na₂SO₄, and the filtrate was concentrated under reduced pressure. The residue was purified by silica gel column chromatography and eluted with PE/EA (4:1) to afford tert-butyl N-methyl-N-[(3R)-1-[2-(methylsulfanyl)quinazolin-6-yl]pyrrolidin-3-yl]carbamate (D146, 220 mg). LCMS: (ES, m/z): 375 [M+H]⁺.

### Synthesis of Intermediate D147

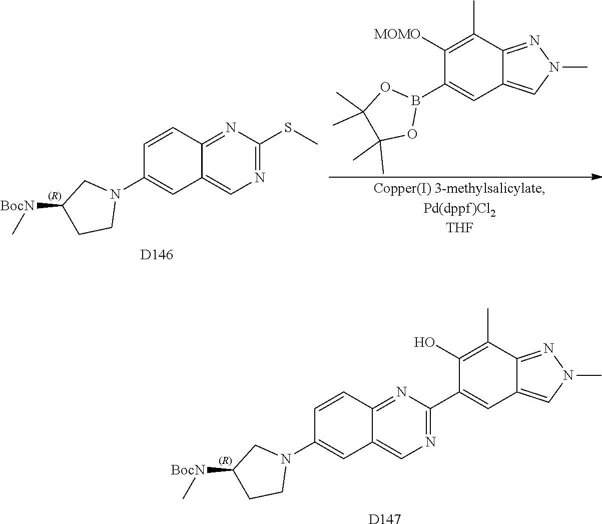

To a stirred solution of Intermediate D146 (220 mg, 0.587 mmol, 1 equiv) and 6-(methoxymethoxy)-2,7-dimethyl-5-(4,4,5,5-tetramethyl-1,3,2-dioxaborolan-2-yl) indazole (195 mg, 0.587 mmol, 1 equiv) in THE (6 mL) were added Copper(I) 3-methylsalicylate (278 mg, 1.291 mmol, 2.2 equiv) and Pd(dppf)Cl₂·CH₂Cl₂ (48 mg, 0.059 mmol, 0.1 equiv) at room temperature under nitrogen atmosphere. The resulting mixture was stirred for 12 h at 90° C. under nitrogen atmosphere and subsequently concentrated under reduced pressure. The residue was purified by reversed-phase flash chromatography following Condition 7, Gradient 5 as previously described to afford tert-butyl N-[(3R)-1-{2-[6-(methoxymethoxy)-2,7-dimethylindazol-5-yl]quinazolin-6-yl}pyrrolidin-3-yl]-N-methylcarbamate (D147, 110 mg). LCMS: (ES, m/z): 489 [M+H]⁺.

### Synthesis of Compound 521

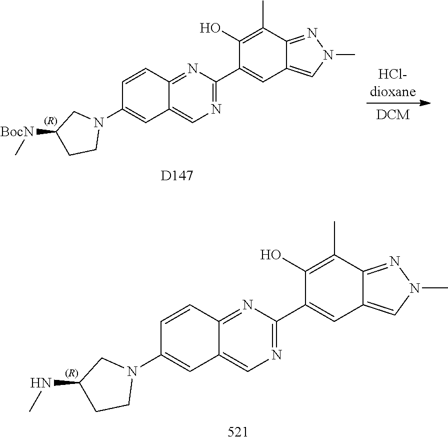

A solution of Intermediate D147 (110 mg, 0.225 mmol, 1 equiv) and HCl (gas) in 1,4-dioxane (1 mL) in DCM (5 mL) was stirred for 2 h at room temperature and then concentrated under reduced pressure. The residue was purified by reversed-phase flash chromatography following Condition 10, Gradient 26 as previously described to afford 2,7-dimethyl-5-{6-[(3R)-3-(methylamino)pyrrolidin-1-yl]quinazolin-2-yl}indazol-6-ol (Compound 521, 28 mg). Absolute stereochemistry of the compound was arbitrarily assigned. LCMS: (ES, m/z): 389 [M+H]⁺. ¹H NMR: (300 MHz, DMSO-d₆) δ 13.70 (s, 1H), 9.47 (s, 1H), 8.86 (s, 1H), 8.38 (s, 1H), 7.90 (d, J=9.1 Hz, 1H), 7.52 (dd, J=9.3, 2.7 Hz, 1H), 6.93 (d, J=2.7 Hz, 1H), 4.14 (s, 3H), 3.63-3.38 (m, 4H), 3.19 (dd, J=9.8, 4.4 Hz, 1H), 2.37 (d, J=16.8 Hz, 6H), 2.16 (dd, J=12.6, 6.1 Hz, 1H), 2.05 (s, 1H), 1.88 (dd, J=11.9, 6.1 Hz, 1H).

### Example 239: Synthesis of Compound 522

### Synthesis of Intermediate D149

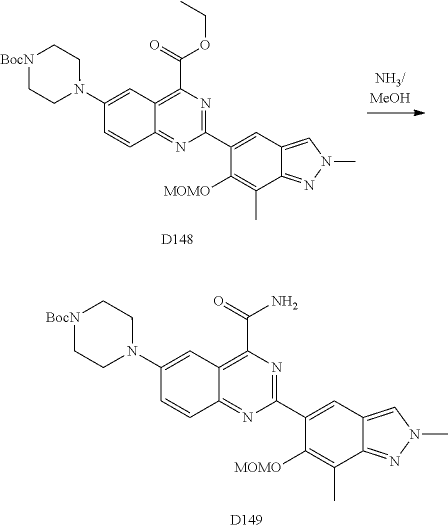

Ethyl 6-[4-(tert-butoxycarbonyl) piperazin-1-yl]-2-[6-(methoxymethoxy)-2,7-dimethylindazol-5-yl]quinazoline-4-carboxylate (D148, 210 mg, 0.356 mmol, 1 equiv) was added to a stirred solution of NH₃(g) in MeOH (8 mL) at room temperature. The resulting mixture was stirred overnight at 80° C. and subsequently concentrated under reduced pressure to afford tert-butyl 4-{4-carbamoyl-2-[6-(methoxymethoxy)-2,7-dimethylindazol-5-yl]quinazolin-6-yl}piperazine-1-carboxylate (D149, 195 mg). LCMS: (ES, m/z): 562 [M+H]⁺.

### Synthesis of Intermediate D50

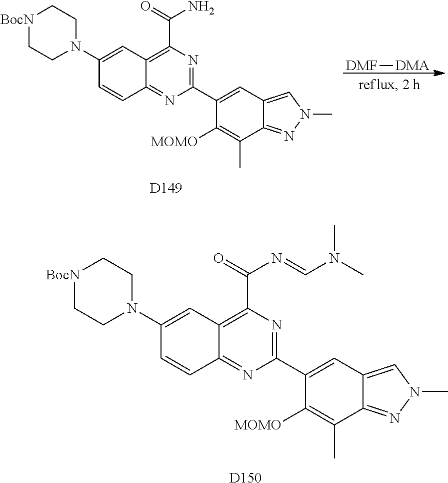

Intermediate D149 (195 mg, 0.347 mmol, 1 equiv) was added to a stirred solution of (dimethoxymethyl) dimethylamine (4 mL, 0.174 mmol). The resulting mixture was stirred for 2 h at 100° C. and then concentrated under reduced pressure to afford tert-butyl 4-(4-{[(1E)-(dimethylamino) methylidene]carbamoyl}-2-[6-(methoxymethoxy)-2,7-dimethylindazol-5-yl]quinazolin-6-yl)piperazine-1-carboxylate (D150, 170 mg). LCMS: (ES, m/z): 617 [M+H]⁺.

### Synthesis of Intermediate D151

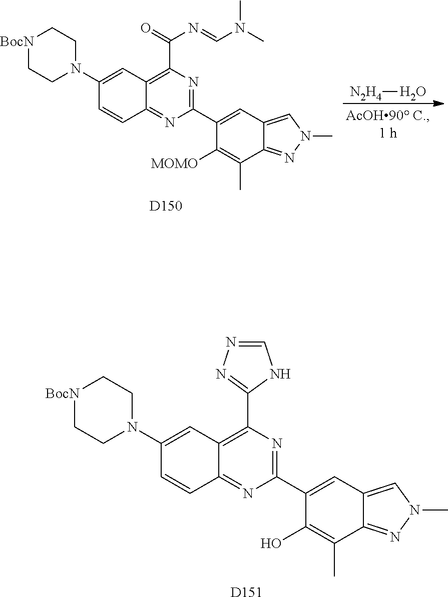

To a stirred solution of Intermediate D150 (170 mg, 0.276 mmol, 1 equiv) in AcOH (4 mL) was added hydrazine hydrate (0.2 mL) at room temperature. The resulting mixture was stirred for 1 h at 90° C. and later concentrated under reduced pressure. The residue was purified by trituration with ACN (3 mL) to afford tert-butyl 4-[2-(6-hydroxy-2,7-dimethylindazol-5-yl)-4-(4H-1,2,4-triazol-3-yl) quinazolin-6-yl]piperazine-1-carboxylate (D151, 132 mg). LCMS: (ES, m/z): 542 [M+H]⁺.

### Synthesis of Compound 522

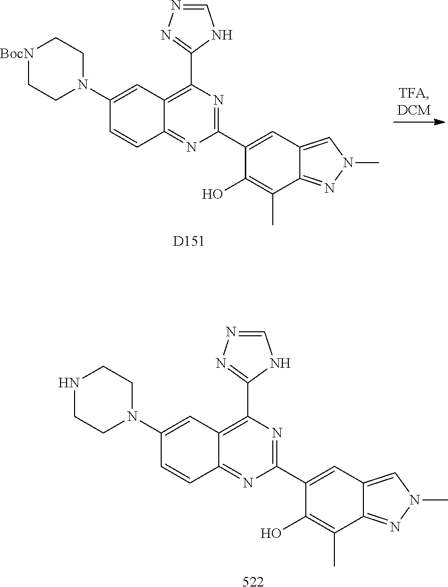 [SMILES: Cc1c(O)c(-c2nc(-c3nnc[nH]3)c3cc(N4CCN(C(=O)OC(C)(C)C)CC4)ccc3n2)cc2cn(C)nc12 ; Cc1c(O)c(-c2nc(-c3nnc[nH]3)c3cc(N4CCNCC4)ccc3n2)cc2cn(C)nc12 ; "D151" ; "522"]

To a stirred solution of Intermediate D151 (75 mg, 0.138 mmol, 1 equiv) in DCM (1 mL) was added TFA (1 mL) at room temperature. The resulting mixture was stirred for 1 h at room temperature and then concentrated under reduced pressure. The crude product was purified by Prep-HPLC following Condition 7, Gradient 5 as previously described to afford 2,7-dimethyl-5-[6-(piperazin-1-yl)-4-(4H-1,2,4-triazol-3-yl) quinazolin-2-yl]indazol-6-ol; bis(trifluoroacetic acid) (Compound 522, 39.3 mL). LCMS: (ES, m/z): 441 [M+H]⁺. ¹H NMR: (400 MHz, DMSO-d₆) δ 13.53-13.48 (s, 1H), 9.43-9.00 (m, 2H), 8.81 (s, 2H), 8.44 (s, 1H), 8.13-8.01 (m, 2H), 4.17 (s, 3H), 3.60 (d, J=10.4 Hz, 1H), 3.37 (s, 4H), 2.42 (s, 3H).

### Example 240: Synthesis of Compound 523

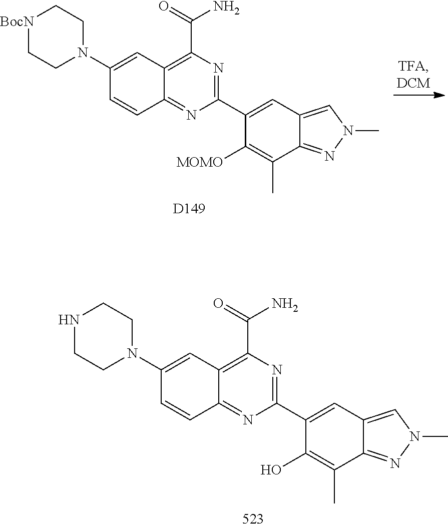

To a stirred solution of Intermediate D149 (85 mg, 0.151 mmol, 1 equiv) in DCM (1 mL) was added TFA (1 mL) at room temperature. The resulting mixture was stirred for 1 h at room temperature and then concentrated under reduced pressure. The crude product was purified by Prep-HPLC following Condition 13, Gradient 6 as previously described to afford 2-(6-hydroxy-2,7-dimethylindazol-5-yl)-6-(piperazin-1-yl) quinazoline-4-carboxamide (Compound 523, 25.9 mg). LCMS: (ES, m/z): 441 [M+H]⁺. ¹H NMR: (400 MHz, DMSO-d₆) δ 13.38 (s, 1H), 9.06 (s, 2H), 8.62 (s, 2H), 8.06 (s, 1H), 7.98 (d, J=2.6 Hz, 2H), 4.16 (s, 3H), 3.31-3.23 (m, 4H), 2.90 (t, J=5.1 Hz, 4H), 2.41 (s, 3H).

### Example 241: Synthesis of Compound 524

### Synthesis of Intermediate D152

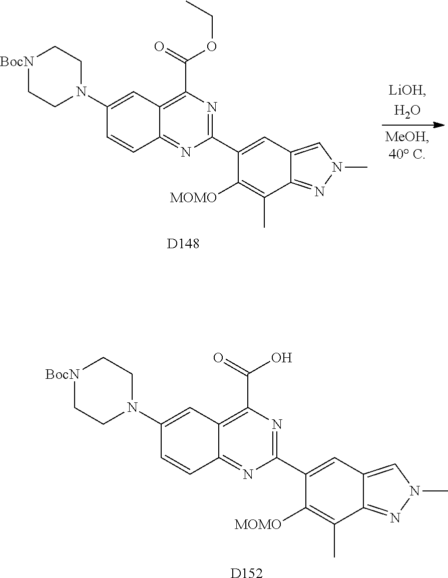

To a stirred solution of Intermediate D148 (100 mg, 0.169 mmol, 1 equiv) in MeOH (2 mL) was added LiOH·H₂O (14.21 mg, 0.338 mmol, 2 equiv) at room temperature. The resulting mixture was stirred overnight at 40° C., then concentrated under vacuum and subsequently diluted with H₂O (5 mL) and acidified to pH 6 with HCl (1N). The aqueous layer was extracted with DCM (3×100 mL), and the combined organic layers were concentrated under reduced pressure to afford 6-[4-(tert-butoxycarbonyl) piperazin-1-yl]-2-[6-(methoxymethoxy)-2,7-dimethylindazol-5-yl]quinazoline-4-carboxylic acid (D152, 80 mg). LCMS: (ES, m/z): 563 [M+H]⁺.

### Synthesis of Compound 524

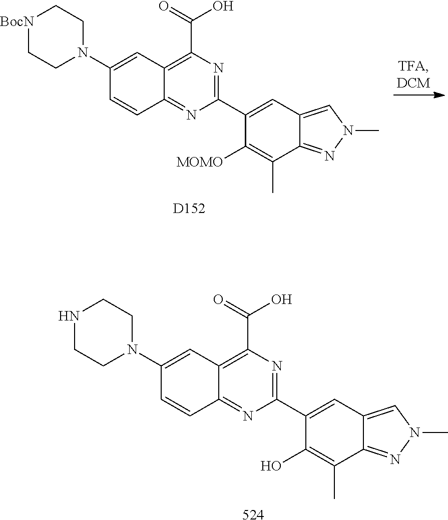

To a stirred solution of Intermediate D152 (80 mg, 0.142 mmol, 1 equiv) in DCM (1 mL) was added TFA (1 mL) at room temperature. The resulting mixture was stirred for 1 h at room temperature and then concentrated under reduced pressure. The crude product was purified by Prep-HPLC following Condition 13, Gradient 1 as previously described to afford 2-(6-hydroxy-2,7-dimethylindazol-5-yl)-6-(piperazin-1-yl) quinazoline-4-carboxylic acid (Compound 524, 51.1 mg, 85.88%) as yellow solid. LCMS: (ES, m/z): 419 [M+H]⁺. ¹H NMR: (400 MHz, DMSO-d₆) δ 13.77 (s, 1H), 10.12 (s, 2H), 8.86 (s, 1H), 8.40 (s, 1H), 7.94 (d, J=1.5 Hz, 2H), 7.47 (s, 1H), 4.14 (s, 3H), 3.59 (d, J=5.8 Hz, 4H), 3.41 (d, J=5.6 Hz, 4H), 2.39 (s, 3H).

### Example 242: Synthesis of Compound 525

### Synthesis of Intermediate D153

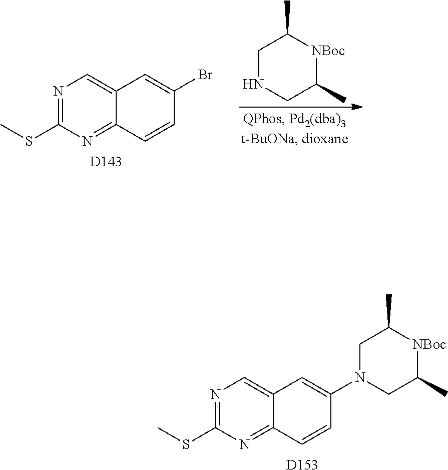

To a stirred solution of Intermediate D143 (400 mg, 1.568 mmol, 1 equiv) and tert-butyl (2R,6S)-2,6-dimethylpiperazine-1-carboxylate (370 mg, 1.725 mmol, 1.1 equiv) in dioxane (15 mL) were added t-BuONa (302 mg, 3.136 mmol, 2 equiv), 1,2,3,4,5-pentaphenyl-1-(di-tert-butylphosphino)ferrocene (223 mg, 0.314 mmol, 0.2 equiv) and Pd₂(dba)₃ (144 mg, 0.157 mmol, 0.1 equiv) at room temperature under nitrogen atmosphere. The resulting mixture was stirred for 4 h at 90° C. under nitrogen atmosphere and subsequently allowed to cool to room temperature. The reaction was quenched by the addition of water (80 mL) at room temperature and then extracted with EtOAc (3×10 mL). The combined organic layers were washed with brine (1×5 mL), dried over anhydrous Na₂SO₄, and the filtrate was concentrated under reduced pressure. The residue was purified by silica gel column chromatography and eluted with PE/EA (3:1) to afford tert-butyl (2R,6S)-2,6-dimethyl-4-[2-(methylsulfanyl)quinazolin-6-yl]piperazine-1-carboxylate (D153, 320 mg). Absolute stereochemistry of the compound was arbitrarily assigned. LCMS: (ES, m/z): 389 [M+H]⁺.

### Synthesis of Intermediate D154

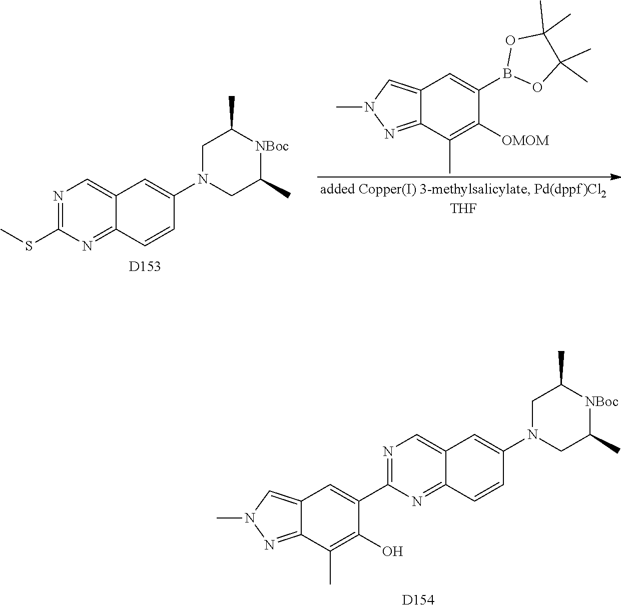

To a stirred solution of Intermediate D153 (320 mg, 0.824 mmol, 1 equiv) and 6-(methoxymethoxy)-2,7-dimethyl-5-(4,4,5,5-tetramethyl-1,3,2-dioxaborolan-2-yl) indazole (274 mg, 0.824 mmol, 1 equiv) in THE (10 mL) were added Copper(I) 3-methylsalicylate (389 mg, 1.813 mmol, 2.2 equiv) and Pd(dppf)Cl₂ (61 mg, 0.082 mmol, 0.1 equiv) at room temperature under nitrogen atmosphere. The resulting mixture was stirred for 12 h at 90° C. under nitrogen atmosphere, then allowed to cool to room temperature, quenched by the addition of water (50 mL) at room temperature, and extracted with EtOAc (3×10 mL). The combined organic layers were washed with brine (1×5 mL), dried over anhydrous Na₂SO₄, and the filtrate was concentrated under reduced pressure. The residue was purified by reversed-phase flash chromatography following Condition 10, Gradient 25 as previously described to afford tert-butyl (2R,6S)-4-{2-[6-(methoxymethoxy)-2,7-dimethylindazol-5-yl]quinazolin-6-yl}-2,6-dimethylpiperazine-1-carboxylate (D154, 140 mg). LCMS: (ES, m/z): 503 [M+H]⁺

### Synthesis of Compound 525

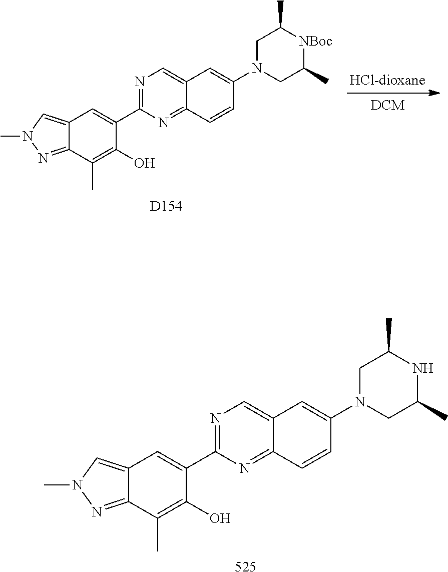

A solution of Intermediate D154 (130 mg, 0.238 mmol, 1 equiv) and HCl (gas) in 1,4-dioxane (2 mL) in DCM (6 mL) was stirred for 2 h at room temperature and then concentrated under reduced pressure. The residue was purified by reversed-phase flash chromatography following Condition 10, Gradient 4 as previously described to afford 5-{6-[(3R,5S)-3,5-dimethylpiperazin-1-yl]quinazolin-2-yl}-2,7-dimethylindazol-6-ol (Compound 525, 57 mg). Absolute stereochemistry of the compound was arbitrarily assigned. LCMS: (ES, m/z): 403 [M+H]⁺. ¹H NMR: (300 MHz, DMSO-d₆) δ 13.64 (s, 1H), 9.52 (s, 1H), 8.90 (s, 1H), 8.40 (s, 1H), 8.01-7.87 (m, 2H), 7.39 (d, J=2.3 Hz, 1H), 4.14 (s, 3H), 3.81 (dd, J=12.0, 2.8 Hz, 2H), 2.90 (dd, J=16.7, 2.8 Hz, 2H), 2.43-2.25 (m, 5H), 1.08 (d, J=6.2 Hz, 6H).

### Example 243: Synthesis of Compound 526

### Synthesis of Intermediate D156

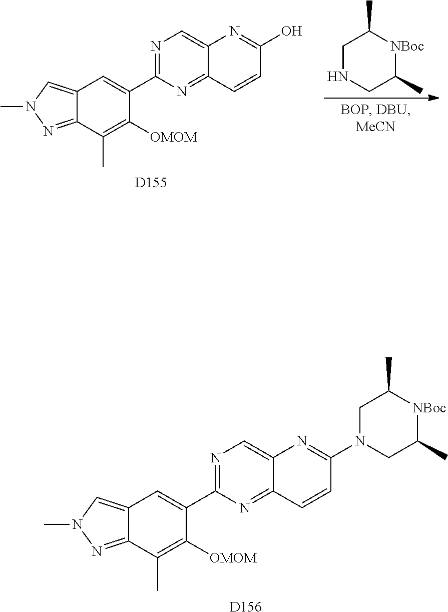

To a stirred solution of 2-[6-(methoxymethoxy)-2,7-dimethylindazol-5-yl]pyrido[3,2-d]pyrimidin-6-ol (D155, 350 mg, 0.996 mmol, 1 equiv) and tert-butyl (2R,6S)-2,6-dimethylpiperazine-1-carboxylate (320 mg, 1.494 mmol, 1.5 equiv) in MeCN (10 mL) were added BOP (881 mg, 1.992 mmol, 2 equiv) and DBU (455 mg, 2.988 mmol, 3 equiv) at room temperature under nitrogen atmosphere. The resulting mixture was stirred for 24 h at 55° C. under nitrogen atmosphere and subsequently allowed to cool to room temperature. The reaction was quenched by the addition of water (50 mL) at room temperature and extracted with EtOAc (3×20 mL). The combined organic layers were washed with brine (1×20 mL), dried over anhydrous Na₂SO₄, and the filtrate was concentrated under reduced pressure. The residue was purified by silica gel column chromatography and eluted with EA to afford tert-butyl (2R,6S)-4-[2-(6-hydroxy-2,7-dimethylindazol-5-yl)pyrido[3,2-d]pyrimidin-6-yl]-2,6-dimethylpiperazine-1-carboxylate (D156, 400 mg). LCMS: (ES, m/z): 548 [M+H]⁺.

### Synthesis of Compound 526

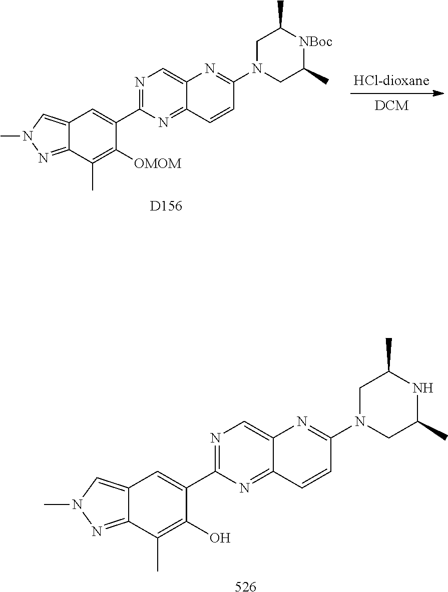

A solution of Intermediate D156 (168 mg, 0.307 mmol, 1 equiv) and 4 M HCl(gas) in 1,4-dioxane (1 mL) in DCM (3 mL) was stirred for 1 h at room temperature and then concentrated under vacuum. The residue was purified by reversed-phase flash chromatography following Condition 10, Gradient 24 as previously described to afford 5-{6-[(3R,5S)-3,5-dimethylpiperazin-1-yl]pyrido[3,2-d]pyrimidin-2-yl}-2,7-dimethylindazol-6-ol (Compound 526, 75 mg). Absolute stereochemistry of the compound was arbitrarily assigned. LCMS: (ES, m/z): 404 [M+H]⁺. ¹H NMR: (300 MHz, DMSO-d₆) δ 13.29 (s, 1H), 9.25 (s, 1H), 8.85 (s, 1H), 8.40 (s, 1H), 8.18 (d, J=9.5 Hz, 1H), 7.81 (d, J=9.6 Hz, 1H), 4.50 (d, J=12.4 Hz, 2H), 4.14 (s, 3H), 2.77 (s, 2H), 2.43 (d, J=23.9 Hz, 5H), 1.08 (d, J=6.1 Hz, 6H).

### Example 244: Synthesis of Compound 527

### Synthesis of Intermediate D158

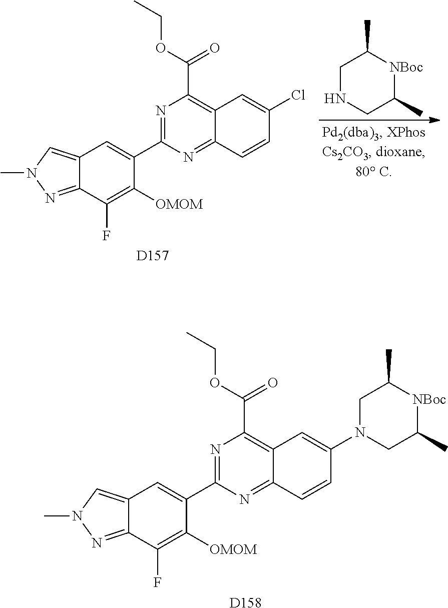

To a solution of ethyl 6-chloro-2-[7-fluoro-6-(methoxymethoxy)-2-methylindazol-5-yl]quinazoline-4-carboxylate (D157, 330.0 mg, 0.742 mmol, 1.0 equiv) and tert-butyl (2R,6S)-2,6-dimethylpiperazine-1-carboxylate (206.6 mg, 0.965 mmol, 1.3 equiv) in dioxane (8 mL) were added Cs₂CO₃ (483.4 mg, 1.484 mmol, 2.0 equiv), Pd₂(dba)₃ (67.9 mg, 0.074 mmol, 0.1 equiv), and XPhos (70.7 mg, 0.148 mmol, 0.2 equiv). After stirring for 3 h at 80° C. under a nitrogen atmosphere, the resulting mixture was concentrated under reduced pressure. The residue was purified by silica gel column chromatography and eluted with PE/EA (5:1) to afford ethyl 6-[(3R,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (D158, 350 mg). LCMS: (ES, m/z): 623 [M+H]⁺.

### Synthesis of Intermediate D159

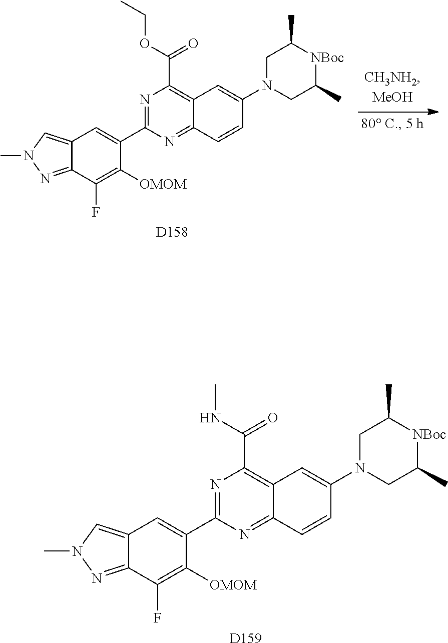

Into a 40 mL vial were added Intermediate D158 (330.0 mg, 0.530 mmol, 1.0 equiv) and methylamine (2M in methanol) (5 mL) at room temperature. The resulting mixture was stirred for 5 h at 80° C. and subsequently concentrated under reduced pressure to afford tert-butyl (2R,6S)-4-{2-[7-fluoro-6-(methoxymethoxy)-2-methylindazol-5-yl]-4-(methylcarbamoyl)quinazolin-6-yl}-2,6-dimethylpiperazine-1-carboxylate (D159, 320 mg). LCMS: (ES, m/z): 608 [M+H]⁺.

### Synthesis of Compound 527

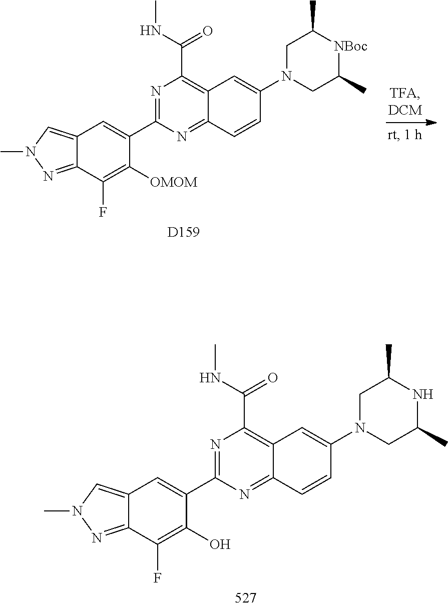

To a stirred solution of Intermediate D159 (150.0 mg, 0.247 mmol, 1.0 equiv) in DCM (2 mL) was added TFA (0.5 mL) dropwise at room temperature. The resulting mixture was stirred for 1 h at room temperature and then concentrated under reduced pressure. The crude product was purified by Prep-HPLC following Condition 14, Gradient 1 as previously described to afford 6-[(3R,5S)-3,5-dimethylpiperazin-1-yl]-2-(7-fluoro-6-hydroxy-2-methylindazol-5-yl)-N-methylquinazoline-4-carboxamide (Compound 527, 53 mg). Absolute stereochemistry of the compound was arbitrarily assigned. LCMS: (ES, m/z): 464 [M+H]⁺. ¹H NMR: (400 MHz, DMSO-d₆) δ 13.42 (s, 1H), 9.23 (d, J=5.1 Hz, 1H), 9.04 (d, J=0.9 Hz, 1H), 8.57 (d, J=2.5 Hz, 1H), 8.17 (t, J=1.7 Hz, 1H), 8.01 (d, J=1.6 Hz, 2H), 4.19 (s, 3H), 3.77 (dd, J=11.9, 2.8 Hz, 2H), 2.97 (d, J=4.6 Hz, 3H), 2.90 (ddd, J=9.8, 6.1, 2.9 Hz, 2H), 2.34 (t, J=11.1 Hz, 2H), 1.09 (d, J=6.3 Hz, 6H).

### Example 245: Synthesis of Compound 531

### Synthesis of Intermediate 161

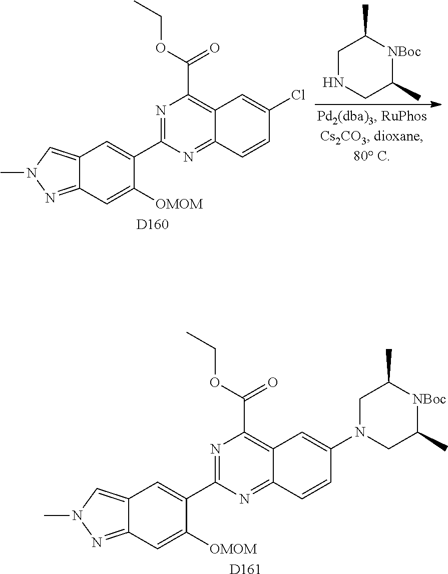

A mixture of ethyl 6-chloro-2-[6-(methoxymethoxy)-2-methylindazol-5-yl]quinazoline-4-carboxylate (D160, 200 mg, 0.469 mmol, 1 equiv), tert-butyl (2R,6S)-2,6-dimethylpiperazine-1-carboxylate (150 mg, 0.704 mmol, 1.50 equiv), Pd₂(dba)₃ (43 mg, 0.047 mmol, 0.10 equiv), RuPhos (44 mg, 0.094 mmol, 0.2 equiv) and Cs₂CO₃ (383 mg, 1.174 mmol, 2.00 equiv) in dioxane (5 mL) was stirred for 2 h at 80° C. under nitrogen atmosphere, allowed to cool to room temperature, and subsequently concentrated under reduced pressure. The residue was purified by silica gel column chromatography and eluted with PE/EA (1:1) to afford ethyl 6-[(3R,5S)-4-(tert-butoxycarbonyl)-3,5-dimethylpiperazin-1-yl]-2-[6-(methoxymethoxy)-2-methylindazol-5-yl]quinazoline-4-carboxylate (D161, 170 mg). LCMS: (ES, m/z): 605 [M+H]⁺.

### Synthesis of Intermediate D162

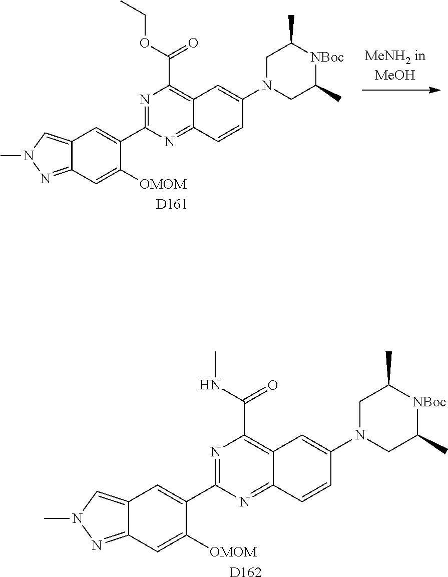

A mixture of 2 M CH₃NH₂ in MeOH (0.5 mL) was treated with Intermediate D161 (50 mg, 0.083 mmol, 1 equiv) for 1 h at 80° C. and subsequently concentrated under reduced pressure to afford tert-butyl (2R,6S)-4-{2-[6-(methoxymethoxy)-2-methylindazol-5-yl]-4-(methylcarbamoyl)quinazolin-6-yl}-2,6-dimethylpiperazine-1-carboxylate, (D162, 45 mg, crude) which was used directly in the next step without further purification. LCMS: (ES, m/z): 590 [M+H]⁺.

### Synthesis of Compound 531

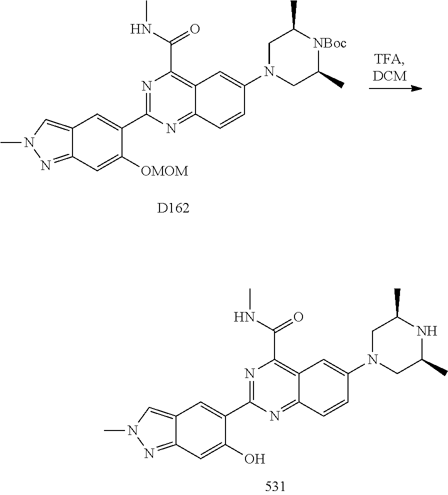

A solution of TFA (0.15 mL) in DCM (0.5 mL) was treated with Intermediate D162 (40 mg, 0.068 mmol, 1 equiv) for 15 min at room temperature and then concentrated under reduced pressure. The residue was purified by reversed-phase flash chromatography following Condition 9, Gradient 9 as previously described to afford 6-[(3R,5S)-3,5-dimethylpiperazin-1-yl]-2-(6-hydroxy-2-methylindazol-5-yl)-N-methylquinazoline-4-carboxamide (Compound 531, 13 mg). Absolute stereochemistry of the compound was arbitrarily assigned. LCMS: (ES, m/z): 446 [M+H]⁺. ¹H NMR: (400 MHz, DMSO-d₆) δ 13.22 (s, 1H), 9.22 (d, J=8.0 Hz, 2H), 8.44 (s, 1H), 8.16 (d, J=2.4 Hz, 1H), 8.06-7.93 (m, 2H), 6.91 (s, 1H), 4.14 (s, 3H), 3.93-3.65 (m, 2H), 2.97 (d, J=4.8 Hz, 3H), 2.90 (s, 2H), 2.32 (t, J=11.0 Hz, 2H), 1.09 (d, J=6.2 Hz, 6H).

### Example 246: Synthesis of Compound 534

### Synthesis of Intermediate D164

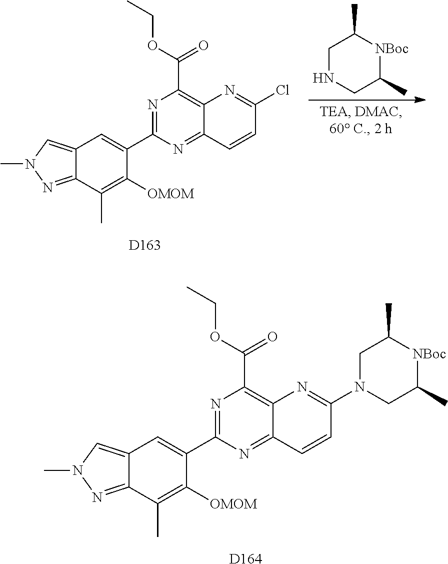

To a stirred mixture of ethyl 6-chloro-2-[6-(methoxymethoxy)-2,7-dimethylindazol-5-yl]pyrido[3,2-d]pyrimidine-4-carboxylate (D163, 120 mg, 0.272 mmol, 1.0 equiv) and tert-butyl (2R,6S)-2,6-dimethylpiperazine-1-carboxylate (67.6 mg, 0.316 mmol, 1.0 equiv) in DMAc (2 mL) was added TEA (95.8 mg, 0.948 mmol, 3.0 equiv) at room temperature under nitrogen atmosphere. The resulting mixture was stirred for 2 h at 60° C. under nitrogen atmosphere, allowed to cool to room temperature, and subsequently concentrated under reduced pressure. The crude resulting mixture (D164) was used in the next step directly without further purification. LCMS: (ES, m/z): 620 [M+H]⁺.

### Synthesis of Intermediate D165

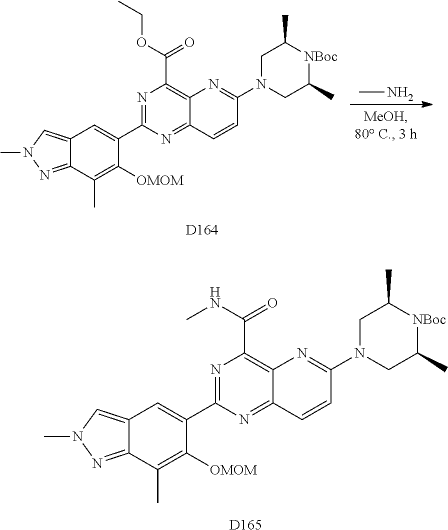

To the above crude was added CH₃NH₂ (1.5 mL, 2M in MeOH) in MeOH (10 mL) at room temperature. The resulting mixture was stirred for an additional 3 h at 80° C., allowed to cool to room temperature, and extracted with EtOAc (3×10 mL). The combined organic layers were washed with water (1×10 mL) and brine (1×10 mL), dried over anhydrous Na₂SO₄, and the filtrate was concentrated under reduced pressure. The residue was purified by Prep-TLC (EA) to afford tert-butyl (2R,6S)-4-{2-[6-(methoxymethoxy)-2,7-dimethylindazol-5-yl]-4-(methylcarbamoyl)pyrido[3,2-d]pyrimidin-6-yl}-2,6-dimethylpiperazine-1-carboxylate (D165, 121 mg). LCMS: (ES, m/z): 577 [M+H]⁺.

### Synthesis of Compound 534

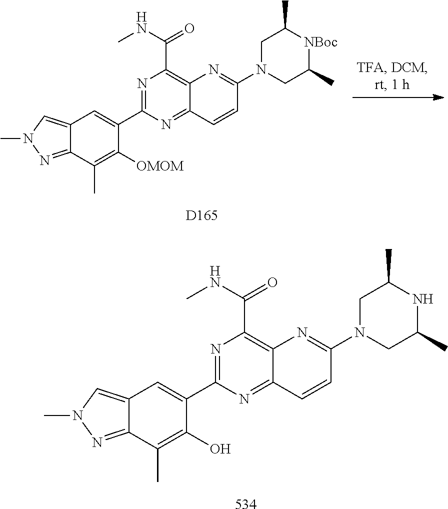

A solution of Intermediate D165 (121 mg, 0.200 mmol, 1.0 equiv) in DCM (1 mL) was treated with TFA (0.25 mL) at room temperature. The resulting mixture was stirred for 1 h at room temperature under nitrogen atmosphere and then concentrated under reduced pressure. The crude product was purified by Prep-HPLC following Condition 6, Gradient 18 as previously described to afford 6-[(3R,5S)-3,5-dimethylpiperazin-1-yl]-2-(6-hydroxy-2,7-dimethylindazol-5-yl)-N-methylpyrido[3,2-d]pyrimidine-4-carboxamide (Compound 534, 60.2 mg). Absolute stereochemistry of the compound was arbitrarily assigned. LCMS: (ES, m/z): 461 [M+H]⁺. ¹H NMR: (300 MHz, DMSO-d₆) δ 13.25 (s, 1H), 8.87 (d, J=4.9 Hz, 1H), 8.60 (d, J=9.5 Hz, 1H), 8.45 (s, 1H), 8.36 (s, 1H), 8.06 (d, J=9.3 Hz, 1H), 4.72 (d, J=12.3 Hz, 2H), 4.14 (s, 3H), 2.92 (d, J=4.7 Hz, 3H), 2.74 (d, J=8.3 Hz, 2H), 2.48 (d, J=2.8 Hz, 2H), 2.37 (s, 3H), 1.08 (d, J=6.2 Hz, 6H).

### Example 247: Synthesis of Compound 535

### Synthesis of Intermediate D129

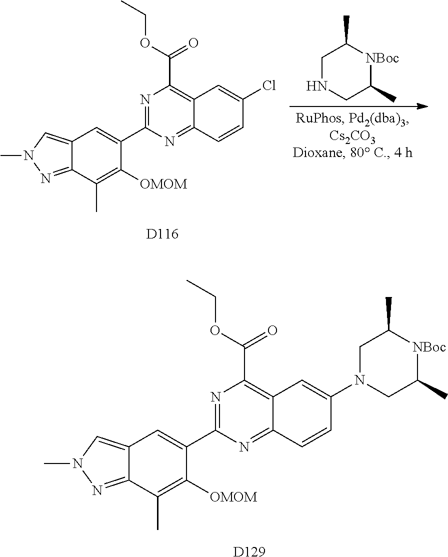

To a stirred mixture of ethyl 6-chloro-2-[6-(methoxymethoxy)-2,7-dimethylindazol-5-yl]quinazoline-4-carboxylate (D116, 600 mg, 1.361 mmol, 1 equiv) and tert-butyl (2R,6S)-2,6-dimethylpiperazine-1-carboxylate (437 mg, 2.039 mmol, 1.50 equiv) in dioxane (12 mL) were added RuPhos (127 mg, 0.272 mmol, 0.20 equiv), Pd₂(dba)₃ (125 mg, 0.137 mmol, 0.10 equiv) and Cs₂CO₃ (890 mg, 2.732 mmol, 2.01 equiv) in portions. The resulting mixture was stirred for 4 h at 80° C. under nitrogen atmosphere and subsequently concentrated under reduced pressure. The residue was purified by silica gel column chromatography and eluted with PE/EA (1:1) to afford ethyl 6-[(3R,5S)-4-(tert-butoxycarbonyl)-3,5-dimethylpiperazin-1-yl]-2-[6-(methoxymethoxy)-2,7-dimethylindazol-5-yl]quinazoline-4-carboxylate (D129, 580 mg). LCMS: (ES, m/z): 619 [M+H]⁺.

### Synthesis of Intermediate D166

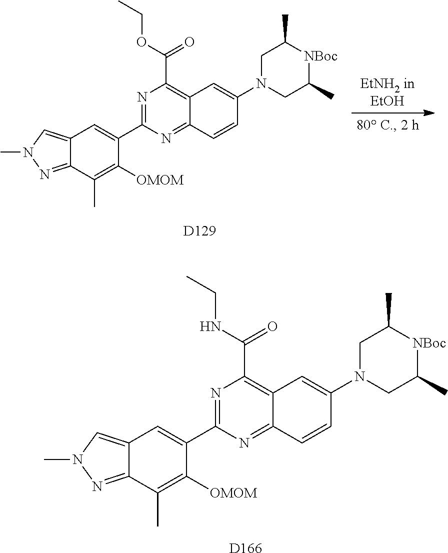

A mixture of Intermediate D129 (100 mg, 0.162 mmol, 1 equiv) and 2M EtNH₂ in EtOH was stirred for 2 h at 80° C. and then concentrated under reduced pressure to afford tert-butyl (2R,6S)-4-[4-(ethylcarbamoyl)-2-[6-(methoxymethoxy)-2,7-dimethylindazol-5-yl]quinazolin-6-yl]-2,6-dimethylpiperazine-1-carboxylate (D166, 85 mg, crude), which was used in the next step directly without further purification. LCMS: (ES, m/z): 618 [M+H]⁺.

### Synthesis of Compound 535

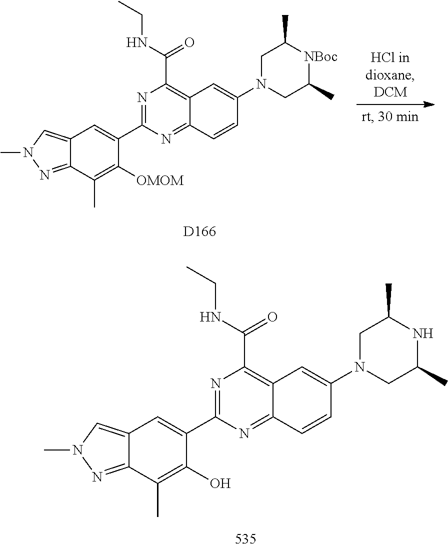

A solution of Intermediate D166 (80 mg, 0.130 mmol, 1 equiv) in DCM (0.8 mL) was treated with 4 M HCl (gas) in 1,4-dioxane (0.4 mL) for 1 h at room temperature and later concentrated under reduced pressure. The residue was purified by reversed-phase flash chromatography following Condition 9, Gradient 5 as previously described to afford 6-[(3R,5S)-3,5-dimethylpiperazin-1-yl]-N-ethyl-2-(6-hydroxy-2,7-dimethylindazol-5-yl)quinazoline-4-carboxamide (Compound 535, 52.9 mg). Absolute stereochemistry of the compound was arbitrarily assigned. LCMS: (ES, m/z): 474 [M+H]⁺. ¹H NMR: (400 MHz, DMSO-d₆) δ 13.41 (s, 1H), 9.24 (t, J=5.9 Hz, 1H), 9.05 (s, 1H), 8.43 (s, 1H), 8.00 (dd, J=4.3, 2.4 Hz, 3H), 4.16 (s, 3H), 3.82-3.71 (m, 2H), 3.55-3.42 (m, 2H), 2.99-2.82 (m, 2H), 2.41 (s, 3H), 2.33 (t, J=11.0 Hz, 2H), 1.26 (t, J=7.2 Hz, 3H), 1.08 (d, J=6.2 Hz, 6H).

### Example 248: Synthesis of Compound 537

### Synthesis of Intermediate D167

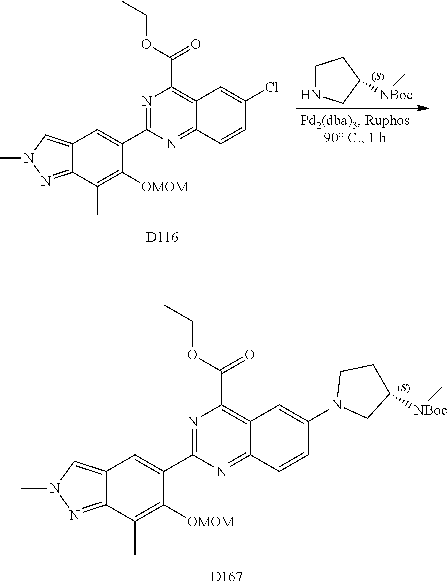

To a solution of ethyl 6-chloro-2-[6-(methoxymethoxy)-2,7-dimethylindazol-5-yl]quinazoline-4-carboxylate (D116, 200 mg, 0.454 mmol, 1 equiv) and tert-butyl N-methyl-N-[(3S)-pyrrolidin-3-yl]carbamate (136 mg, 0.681 mmol, 1.5 equiv) in dioxane (4 mL) were added Cs₂CO₃ (103 mg, 1.362 mmol, 3.0 equiv), RuPhos (42 mg, 0.091 mmol, 0.2 equiv) and Pd₂(dba)₃ (41 mg, 0.045 mmol, 0.1 equiv). After stirring for 1 h at 90° C. under a nitrogen atmosphere, the resulting mixture was concentrated under reduced pressure. The residue was purified by reversed-phase flash chromatography following Condition 10, Gradient 8 as previously described to afford ethyl 6-[(3S)-3-[(tert-butoxycarbonyl)(methyl)amino]pyrrolidin-1-yl]-2-[6-(methoxymethoxy)-2,7-dimethylindazol-5-yl]quinazoline-4-carboxylate (D167, 180 mg). LCMS: (ES, m/z): 591 [M-Et+Me+H]⁺.

### Synthesis of Intermediate D168

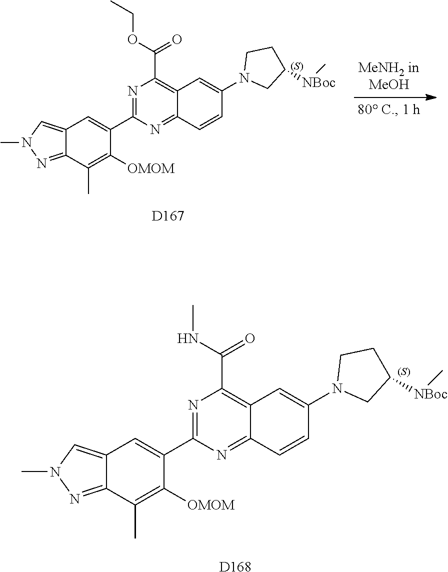

A solution of Intermediate D167 (180 mg, 0.298 mmol, 1 equiv) in CH₃NH₂ in MeOH (4 mL) was stirred for 1 h at 80° C. and subsequently concentrated under reduced pressure to afford tert-butyl (S)-(1-(2-(6-(methoxymethoxy)-2,7-dimethyl-2H-indazol-5-yl)-4-(methylcarbamoyl)quinazolin-6-yl)pyrrolidin-3-yl)(methyl)carbamate (D168, 190 mg, crude), which was used in the next step directly without further purification. LCMS: (ES, m/z): 590 [M+H]⁺.

### Synthesis of Compound 537

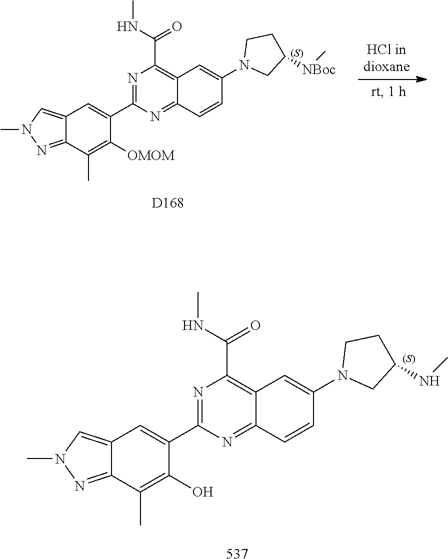

A solution of Intermediate D168 (150 mg, 0.254 mmol, 1 equiv) and 4M HCl (gas) in 1,4-dioxane (4 mL) was stirred for 1 h at room temperature and then concentrated under reduced pressure. The residue was purified by reversed-phase flash chromatography following Condition 10, Gradient 9 as previously described to afford 2-(6-hydroxy-2,7-dimethylindazol-5-yl)-N-methyl-6-[(3S)-3-(methylamino)pyrrolidin-1-yl]quinazoline-4-carboxamide (Compound 537, 52 mg). Absolute stereochemistry of the compound was arbitrarily assigned. LCMS: (ES, m/z): 446 [M+H]⁺. ¹H NMR: (300 MHz, DMSO-d₆) δ 13.48 (s, 1H), 9.15 (d, J=4.9 Hz, 1H), 9.05 (s, 1H), 8.40 (s, 1H), 7.98 (d, J=9.2 Hz, 1H), 7.73 (d, J=2.7 Hz, 1H), 7.59 (dd, J=9.3, 2.7 Hz, 1H), 4.16 (s, 3H), 3.65-3.43 (m, 4H), 3.23 (dd, J=10.1, 4.2 Hz, 1H), 2.96 (d, J=4.8 Hz, 3H), 2.39 (d, J=8.0 Hz, 6H), 2.19 (dd, J=12.8, 6.3 Hz, 1H), 1.99-1.89 (m, 1H).

### Example 249: Synthesis of Compound 539

### Synthesis of Intermediate D169

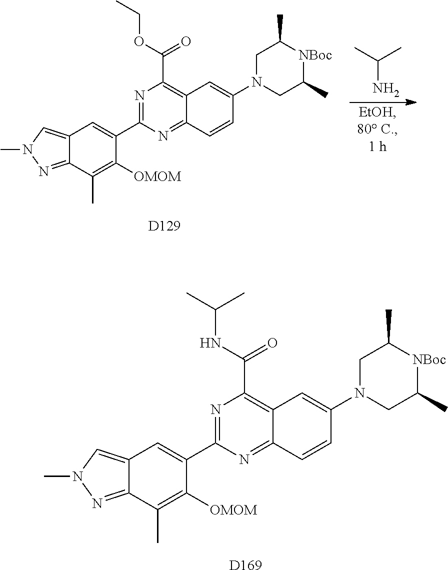

A solution of propan-2-amine (1 mL) in EtOH (2 mL) was treated with ethyl 6-[(3R,5S)-4-(tert-butoxy-carbonyl)-3,5-dimethylpiperazin-1-yl]-2-[6-(methoxymethoxy)-2,7-dimethylindazol-5-yl]quinazoline-4-carboxylate (D129, 100 mg, 0.162 mmol, 1 equiv) for 1 h at 80° C. and then allowed to cool to room temperature. The resulting mixture was concentrated under reduced pressure to afford tert-butyl (2R,6S)-4-[4-(isopropylcarbamoyl)-2-[6-(methoxymethoxy)-2,7-dimethylindazol-5-yl]quinazolin-6-yl]-2,6-dimethylpiperazine-1-carboxylate (D169, 95 mg). LCMS: (ES, m/z): 632 [M+H]⁺.

### Synthesis of Compound 539

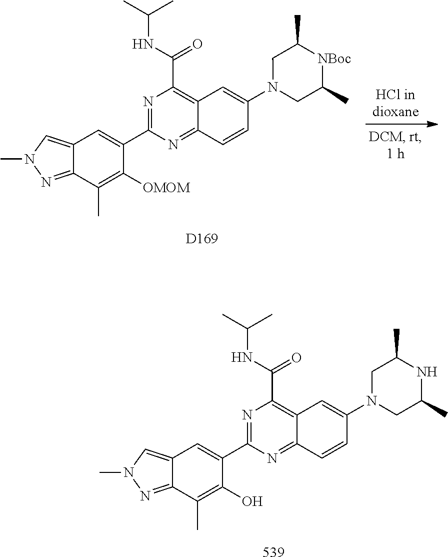

A solution of Intermediate D169 (95 mg, 0.150 mmol, 1 equiv) in DCM (2 mL) was treated with 4 M HCl (gas) in 1,4-dioxane (0.5 mL) for 1 h at room temperature and subsequently concentrated under reduced pressure. The crude product was purified by reversed-phase flash chromatography following Condition 9, Gradient 5 as previously described to afford 6-[(3R,5S)-3,5-dimethylpiperazin-1-yl]-2-(6-hydroxy-2,7-dimethylindazol-5-yl)-N-isopropylquinazoline-4-carboxamide (Compound 539, 68.1 mg). Absolute stereochemistry of the compound was arbitrarily assigned. LCMS: (ES, m/z): 488 [M+H]⁺. ¹H NMR: (400 MHz, DMSO-d₆) δ 13.40 (s, 1H), 9.12-8.84 (m, 2H), 8.45 (s, 1H), 8.00 (d, J=2.4 Hz, 2H), 7.66 (s, 1H), 4.40-4.20 (m, 1H), 4.14 (s, 3H), 3.75 (d, J=11.5 Hz, 2H), 2.92 (s, 2H), 2.41 (s, 3H), 2.34 (t, J=10.9 Hz, 2H), 1.30 (d, J=6.5 Hz, 6H), 1.08 (d, J=6.4 Hz, 6H).

### Example 250: Synthesis of Compound 540

### Synthesis of Intermediate D170

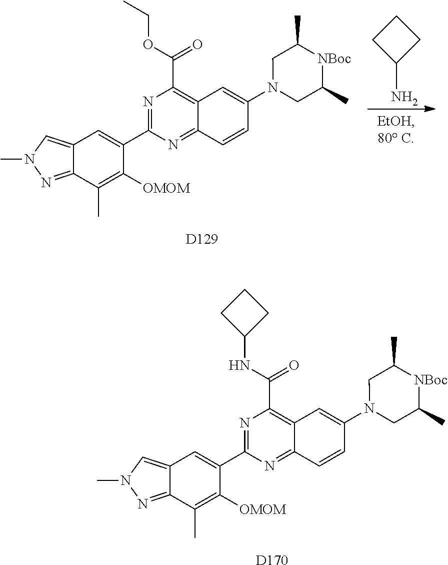

A solution of cyclobutylamine (0.5 mL) in EtOH (2 mL) was treated with Intermediate D129 (100 mg, 0.162 mmol, 1 equiv) for 1 h at 80° C., allowed to cool to room temperature, and then concentrated under reduced pressure to afford tert-butyl (2R,6S)-4-[4-(cyclobutylcarbamoyl)-2-[6-(methoxymethoxy)-2,7-dimethylindazol-5-yl]quinazolin-6-yl]-2,6-dimethylpiperazine-1-carboxylate (D170, 90 mg, 86.50%). LCMS: (ES, m/z): 644 [M+H]⁺.

### Synthesis of Compound 540

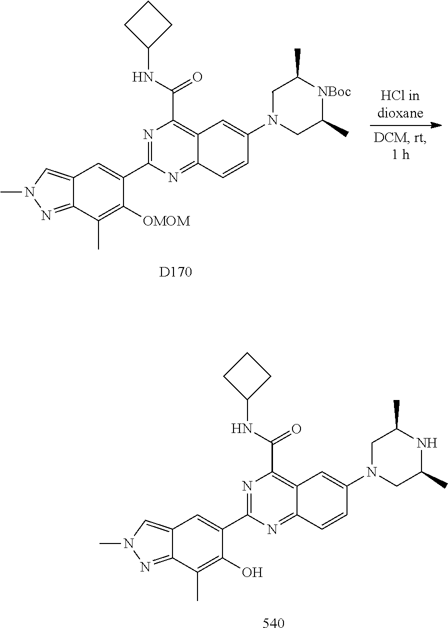

A solution of Intermediate D170 (90 mg, 0.140 mmol, 1 equiv) in DCM (1.2 mL) was treated with 4 M HCl (gas) in 1,4-dioxane (0.4 mL) for 1 h at room temperature and then concentrated under reduced pressure. The crude product was purified by reversed-phase flash chromatography following Condition 9, Gradient 5 as previously described to afford 6-[4-(azetidin-1-yl)piperidin-1-yl]-2-(6-hydroxy-2,7-dimethylindazol-5-yl)-N-methylquinazoline-4-carboxamide (Compound 540, 53.9 mg). Absolute stereochemistry of the compound was arbitrarily assigned. LCMS: (ES, m/z): 500 [M+H]⁺. ¹H NMR: (400 MHz, DMSO-d₆) δ 13.39 (s, 1H), 9.34 (d, J=8.0 Hz, 1H), 9.01 (s, 1H), 8.45 (s, 1H), 8.00 (d, J=2.0 Hz, 2H), 7.76 (s, 1H), 4.59 (h, J=8.4 Hz, 1H), 4.16 (s, 3H), 3.76 (d, J=11.5 Hz, 2H), 2.93 (s, 2H), 2.41 (s, 3H), 2.40-2.31 (m, 4H), 2.30-2.21 (m, 2H), 1.85-1.69 (m, 2H), 1.09 (d, J=6.2 Hz, 6H).

### Example 251: Synthesis of Compound 541

### Synthesis of Intermediate D171

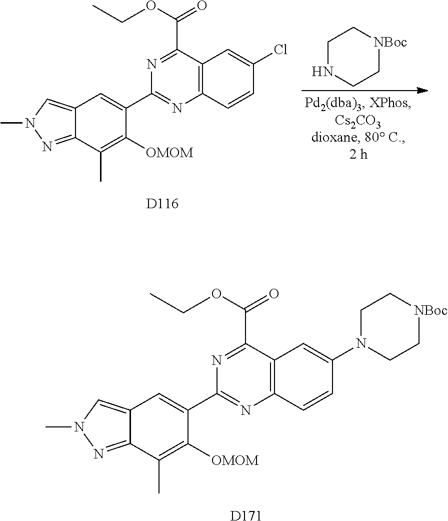

To a stirred solution of Intermediate D116 (0.63 g, 3.402 mmol, 1.5 equiv) in dioxane (20 mL) were added XPhos (0.22 g, 0.454 mmol, 0.2 equiv) and Pd₂(dba)₃ (0.21 g, 0.227 mmol, 0.1 equiv) at room temperature under nitrogen atmosphere. The resulting mixture was stirred for 2 h at 80° C. under nitrogen atmosphere, allowed to cool to room temperature, quenched by the addition of water (100 mL) at room temperature, and then extracted with EtOAc (3×30 mL). The combined organic layers were washed with brine (1×50 mL), dried over anhydrous Na₂SO₄, and the filtrate was concentrated under reduced pressure. The residue was purified by reversed-phase flash chromatography following Condition 9, Gradient 8 as previously described to afford ethyl 6-[4-(tert-butoxycarbonyl)piperazin-1-yl]-2-[6-(methoxymethoxy)-2,7-dimethylindazol-5-yl]quinazoline-4-carboxylate (D171, 1.1 g). LCMS: (ES, m/z): 591 [M+H]⁺.

### Synthesis of Intermediate D172

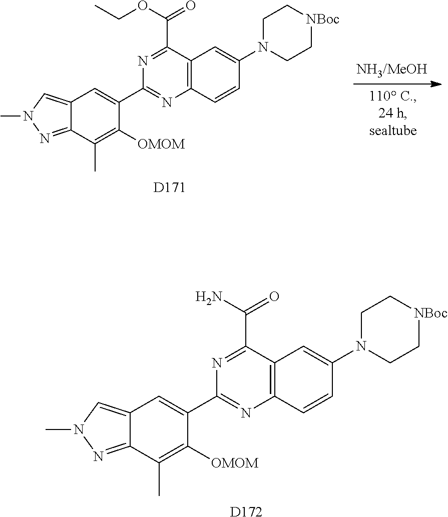

A solution of Intermediate D171 (600 mg, 1.016 mmol, 1 equiv) in 9 M NH₃(g) in MeOH (150 mL) was stirred for 24 h at 110° C., allowed to cool to room temperature and subsequently concentrated under vacuum. The residue was purified by reversed-phase flash chromatography following Condition 9, Gradient 7 as previously described to afford tert-butyl 4-{4-carbamoyl-2-[6-(methoxymethoxy)-2,7-dimethylindazol-5-yl]quinazolin-6-yl}piperazine-1-carboxylate (D172, 454 mg). LCMS: (ES, m/z): 562 [M+H]⁺.

### Synthesis of Intermediate D173

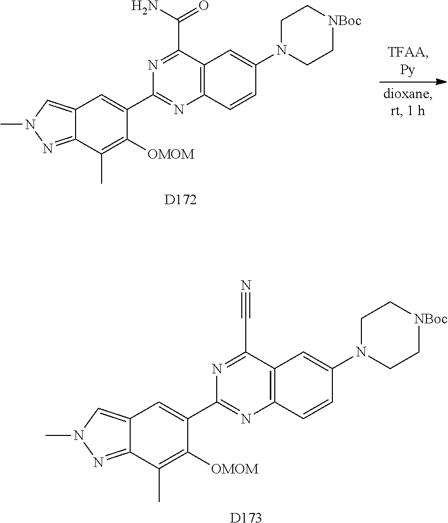

To a stirred solution of Intermediate D172 (450 mg, 0.801 mmol, 1 equiv) and pyridine (76 mg, 0.961 mmol, 1.2 equiv) in dioxane (10 mL) was added TFAA (337 mg, 1.602 mmol, 2 equiv) dropwise at 0° C. under nitrogen atmosphere. The resulting mixture was stirred for 1 h at room temperature under nitrogen atmosphere, quenched by the addition of water (30 mL) at room temperature, and then extracted with EtOAc (3×20 mL). The combined organics were washed with brine (1×30 mL), dried over anhydrous Na₂SO₄, and the filtrate was concentrated under reduced pressure. The residue was purified by reversed-phase flash chromatography following Condition 9, Gradient 6 as previously described to afford tert-butyl 4-{4-cyano-2-[6-(methoxymethoxy)-2,7-dimethylindazol-5-yl]quinazolin-6-yl}piperazine-1-carboxylate (D173, 145 mg). LCMS: (ES, m/z): 544 [M+H]⁺.

### Synthesis of Intermediate D174

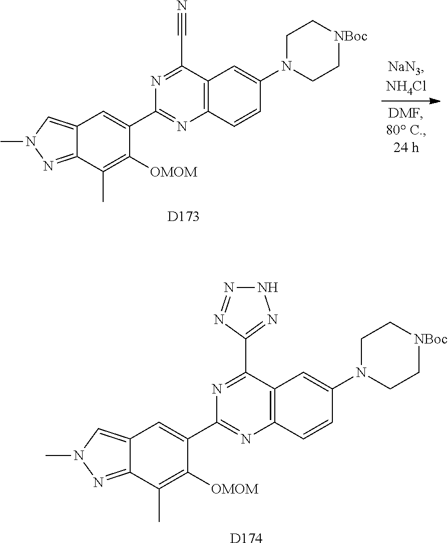

To a stirred solution of Intermediate D173 (360 mg, 0.662 mmol, 1 equiv) and NH₄Cl (106 mg, 1.986 mmol, 3 equiv) in DMF (5 mL) was added NaN₃ (129 mg, 1.986 mmol, 3 equiv) at room temperature under nitrogen atmosphere. The resulting mixture was stirred for 24 h at 80° C. under nitrogen atmosphere and allowed to cool to room temperature. The reaction was quenched by the addition of water (20 mL) at room temperature and then extracted with EtOAc (3×10 mL). The combined organic layers were washed with brine (1×10 mL), dried over anhydrous Na₂SO₄, and the filtrate was concentrated under reduced pressure. The residue was purified by reversed-phase flash chromatography following Condition 10, Gradient 23 as previously described to afford tert-butyl 4-{2-[6-(methoxymethoxy)-2,7-dimethylindazol-5-yl]-4-(2H-1,2,3,4-tetrazol-5-yl)quinazolin-6-yl}piperazine-1-carboxylate (D174, 230 mg). LCMS: (ES, m/z): 587 [M+H]⁺.

### Synthesis of Compound 541

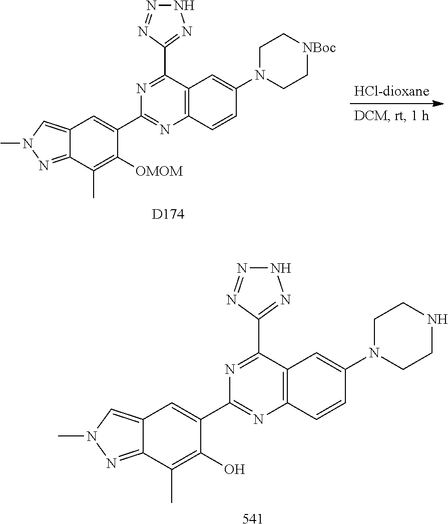

A solution of Intermediate D174 (200 mg, 0.341 mmol, 1 equiv) and 4 M HCl (gas) in 1,4-dioxane (1 mL) in DCM (3 mL) was stirred for 1 h at room temperature under nitrogen atmosphere, concentrated under vacuum, and subsequently basified to pH 8 with saturated NaHCO₃ (aq.). The precipitated solids were collected by filtration and washed with water (3×10 mL) to afford 2,7-dimethyl-5-[6-(piperazin-1-yl)-4-(2H-1,2,3,4-tetrazol-5-yl)quinazolin-2-yl]indazol-6-ol (Compound 541, 76 mg). LCMS: (ES, m/z): 443 [M+H]⁺. ¹H NMR: (400 MHz, DMSO-d₆) δ 14.14 (s, 1H), 9.03 (s, 1H), 8.95 (s, 1H), 8.41 (s, 1H), 7.93 (d, J=1.7 Hz, 2H), 4.15 (s, 3H), 3.34 (s, 4H), 3.06-3.00 (m, 4H), 2.42 (s, 3H).

### Example 252: Synthesis of Compound 542

### Synthesis of Intermediate D176

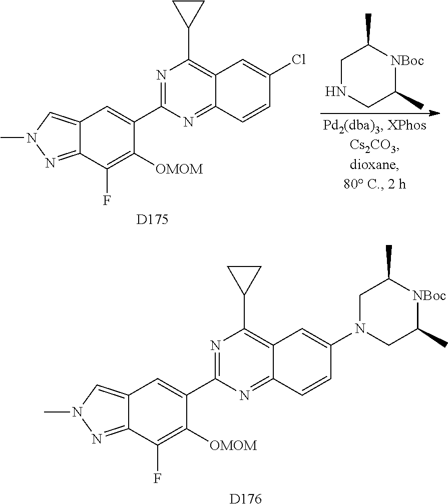

To a stirred mixture of 6-chloro-4-cyclopropyl-2-[7-fluoro-6-(methoxymethoxy)-2-methylindazol-5-yl]quinazoline (D175, 230 mg, 0.557 mmol, 1 equiv) and tert-butyl (2R,6S)-2,6-dimethylpiperazine-1-carboxylate (143.3 mg, 0.668 mmol, 1.2 equiv) in dioxane (5.75 mL) were added Cs₂CO₃ (544.5 mg, 1.671 mmol, 3 equiv), XPhos (53.1 mg, 0.111 mmol, 0.2 equiv) and Pd₂(dba)₃ (51.0 mg, 0.056 mmol, 0.1 equiv) at room temperature under nitrogen atmosphere. The resulting mixture was stirred for 2 h at 80° C. under nitrogen atmosphere and then extracted with EtOAc (3×10 mL). The combined organic layers were washed with water (2×20 mL), dried over anhydrous Na₂SO₄, and the filtrate was concentrated under reduced pressure. The residue was purified by silica gel column chromatography and eluted with EA to afford tert-butyl (2R,6S)-4-{4-cyclopropyl-2-[7-fluoro-6-(methoxymethoxy)-2-methylindazol-5-yl]quinazolin-6-yl}-2,6-dimethylpiperazine-1-carboxylate (D176, 250 mg). LCMS: (ES, m/z): 591 [M+H]⁺.

### Synthesis of Compound 542

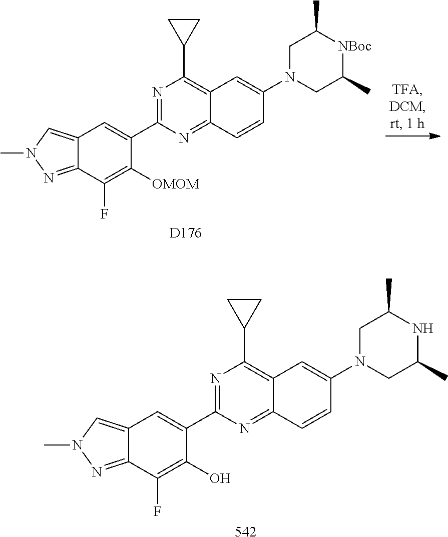

To a stirred mixture of Intermediate D176 (150 mg, 0.254 mmol, 1 equiv) in DCM (3 mL) was added TFA (0.6 mL) at room temperature. The resulting mixture was stirred for 1 h at room temperature, concentrated under vacuum, and the crude product was purified by Prep-HPLC following Condition 6, Gradient 17 as previously described to afford 5-{4-cyclopropyl-6-[(3R,5S)-3,5-dimethylpiperazin-1-yl]quinazolin-2-yl}-7-fluoro-2-methylindazol-6-ol (Compound 542, 62 mg). Absolute stereochemistry of the compound was arbitrarily assigned. LCMS: (ES, m/z): 447 [M+H]⁺. ¹H NMR: (400 MHz, DMSO-d₆) δ 13.93 (s, 1H), 8.72 (d, J=0.9 Hz, 1H), 8.52 (d, J=2.6 Hz, 1H), 7.87 (d, J=2.5 Hz, 2H), 7.61-7.57 (m, 1H), 4.16 (s, 3H), 3.86 (dd, J=12.0, 2.8 Hz, 2H), 3.14 (dt, J=8.1, 3.5 Hz, 1H), 2.90 (ddd, J=9.6, 6.6, 2.9 Hz, 2H), 2.30 (t, J=11.1 Hz, 2H), 1.40 (dt, J=5.2, 3.0 Hz, 2H), 1.34 (dt, J=8.2, 3.2 Hz, 2H), 1.09 (d, J=6.2 Hz, 6H).

### Example 253: Synthesis of Compound 543

### Synthesis of Intermediate D177

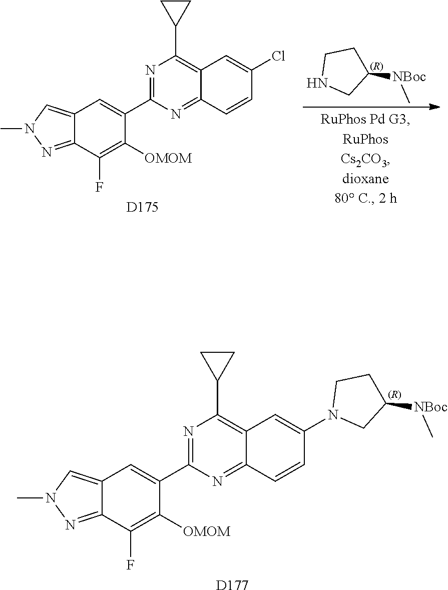

To a stirred mixture of Intermediate D175 (200 mg, 0.484 mmol, 1 equiv) and tert-butyl N-methyl-N-[(3R)-pyrrolidin-3-yl]carbamate (116.4 mg, 0.581 mmol, 1.2 equiv) in dioxane (5 mL) were added Cs₂CO₃ (473.5 mg, 1.452 mmol, 3 equiv), RuPhos (45.2 mg, 0.097 mmol, 0.2 equiv) and RuPhos Palladacycle Gen.3 (40.5 mg, 0.048 mmol, 0.1 equiv) at room temperature under nitrogen atmosphere. The resulting mixture was stirred for 2 h at 80° C. under nitrogen atmosphere and subsequently extracted with EtOAc (3×5 mL). The combined organic layers were washed with water (2×10 mL), dried over anhydrous Na₂SO₄, and the filtrate was concentrated under reduced pressure. The residue was purified by silica gel column chromatography and eluted with PE/EA (1:1) to afford tert-butyl N-[(3R)-1-{4-cyclopropyl-2-[7-fluoro-6-(methoxymethoxy)-2-methylindazol-5-yl]quinazolin-6-yl}pyrrolidin-3-yl]-N-methylcarbamate (Intermediate D177, 250 mg). LCMS: (ES, m/z): 577 [M+H]⁺.

### Synthesis of Compound 543

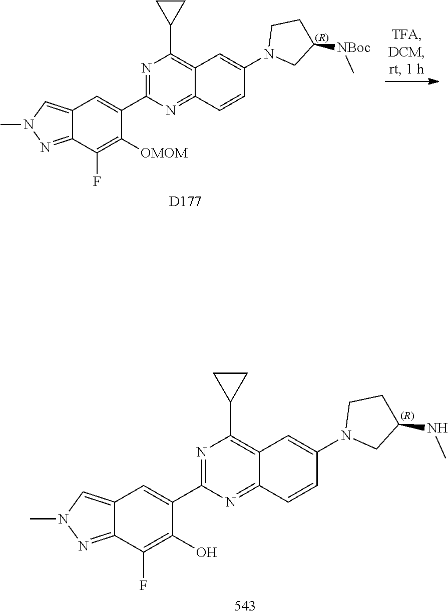

To a stirred mixture of Intermediate D177 (150 mg, 0.260 mmol, 1 equiv) in DCM (3 mL) was added TFA (0.6 mL) at room temperature. The resulting mixture was stirred for 1 h at room temperature and subsequently concentrated under reduced pressure. The crude product was purified by Prep-HPLC following Condition 6, Gradient 17 as previously described to afford 5-{4-cyclopropyl-6-[(3R)-3-(methylamino)pyrrolidin-1-yl]quinazolin-2-yl}-7-fluoro-2-methylindazol-6-ol (Compound 543, 50 mg). Absolute stereochemistry of the compound was arbitrarily assigned. LCMS: (ES, m/z): 433 [M+H]⁺. ¹H NMR: (400 MHz, DMSO-d₆) δ 14.03 (s, 1H), 8.71 (s, 1H), 8.52 (d, J=2.7 Hz, 1H), 7.88 (d, J=9.2 Hz, 1H), 7.50 (dd, J=9.2, 2.5 Hz, 1H), 7.18 (d, J=2.6 Hz, 1H), 4.16 (s, 3H), 3.62 (dd, J=9.9, 5.9 Hz, 1H), 3.53 (t, J=7.4 Hz, 1H), 3.46 (q, J=8.5, 7.8 Hz, 1H), 3.36 (m, 1H), 3.23 (dd, J=10.0, 4.5 Hz, 1H), 3.06 (dd, J=8.8, 4.3 Hz, 1H), 2.35 (s, 3H), 2.17 (dd, J=12.4, 6.2 Hz, 1H), 1.89 (q, J=6.4, 5.8 Hz, 1H), 1.39 (q, J=3.4 Hz, 2H), 1.33 (dt, J=8.3, 3.3 Hz, 2H).

### Example 254: Synthesis of Compound 706

### Synthesis of Intermediate D178

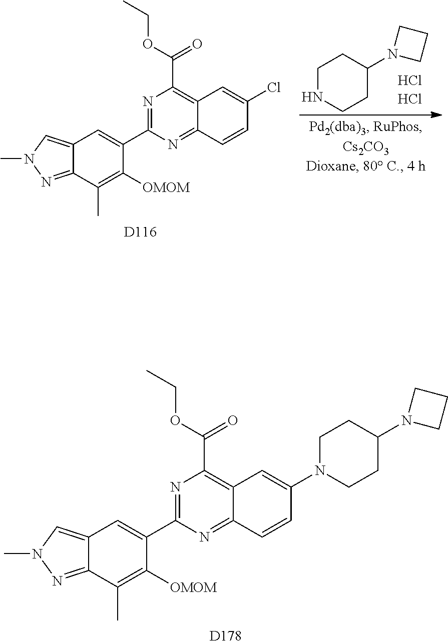

To a stirred mixture of Intermediate D116 (100 mg, 0.227 mmol, 1 equiv) and 4-(azetidin-1-yl)piperidine dihydrochloride (60 mg, 0.271 mmol, 1.19 equiv) in dioxane (2 mL) were added RuPhos (21 mg, 0.045 mmol, 0.20 equiv), Pd₂(dba)₃ (21 mg, 0.023 mmol, 0.10 equiv) and Cs₂CO₃ (148 mg, 0.454 mmol, 2.00 equiv) at room temperature. The resulting mixture was stirred for 4 h at 80° C. under nitrogen atmosphere and then concentrated under reduced pressure. The residue was purified by silica gel column chromatography and eluted with PE/EA (1:1) to afford ethyl 6-[4-(azetidin-1-yl)piperidin-1-yl]-2-[6-(methoxymethoxy)-2,7-dimethylindazol-5-yl]quinazoline-4-carboxylate (D178, 95 mg). LCMS: (ES, m/z): 545 [M+H]⁺.

### Synthesis of Intermediate D179

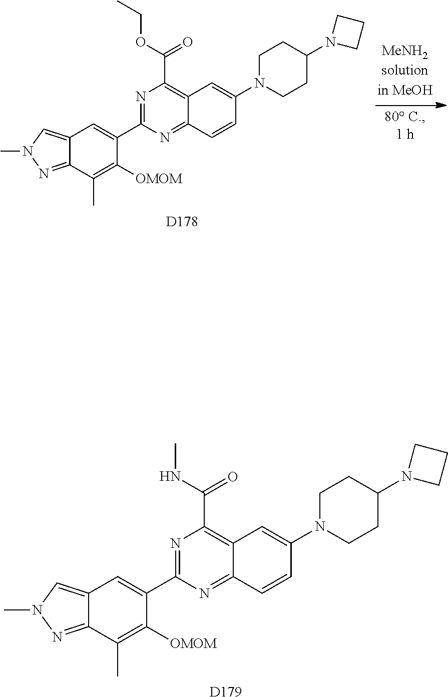

A mixture of Intermediate D178 (95 mg, 0.174 mmol, 1 equiv) and 2M MeNH₃ in MeOH (1 mL) was stirred for 1 h at 80° C. and subsequently concentrated under reduced pressure to afford 6-[4-(azetidin-1-yl)piperidin-1-yl]-2-[6-(methoxymethoxy)-2,7-dimethylindazol-5-yl]-N-methylquinazoline-4-carboxamide (D179, 90 mg). LCMS: (ES, m/z): 530 [M+H]⁺.

### Synthesis of Compound 706

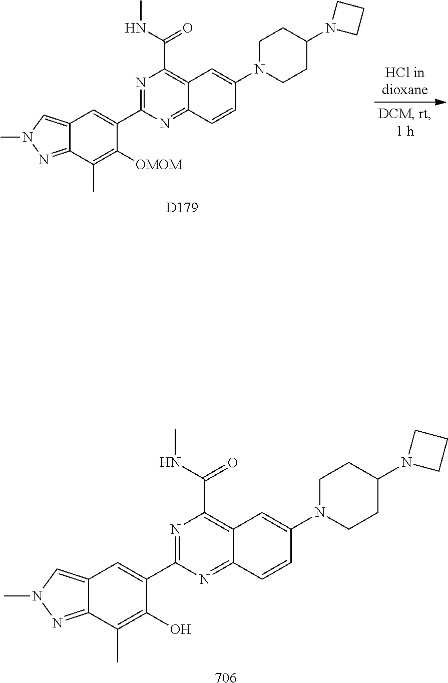

A solution of Intermediate D179 (90 mg, 0.170 mmol, 1 equiv) in DCM (0.8 mL) was treated with 4 M HCl (gas) in 1,4-dioxane (0.4 mL) for 1 h at room temperature and then concentrated under reduced pressure. The crude product was purified by reversed-phase flash chromatography following Condition 9, Gradient 5 as previously described to afford 6-[(3R,5S)-3,5-dimethylpiperazin-1-yl]-N-ethyl-2-(6-hydroxy-2,7-dimethylindazol-5-yl)quinazoline-4-carboxamide (Compound 706, 52.9 mg). LCMS: (ES, m/z): 486 [M+H]⁺. ¹H NMR: (400 MHz, DMSO-d₆) δ 13.40 (s, 1H), 9.18 (d, J=5.0 Hz, 1H), 9.08 (s, 1H), 8.41 (s, 1H), 8.13 (d, J=1.7 Hz, 1H), 7.96 (d, J=1.5 Hz, 2H), 4.16 (s, 3H), 3.72 (d, J=12.9 Hz, 2H), 3.13 (s, 4H), 3.04 (td, J=11.0, 10.3, 5.6 Hz, 2H), 2.96 (d, J=4.7 Hz, 3H), 2.41 (s, 3H), 2.25 (s, 1H), 1.95 (p, J=6.9 Hz, 2H), 1.77 (d, J=12.8 Hz, 2H), 1.31 (q, J=9.2 Hz, 2H).

### Example 255: Synthesis of Compound 707

### Synthesis of Intermediate D157

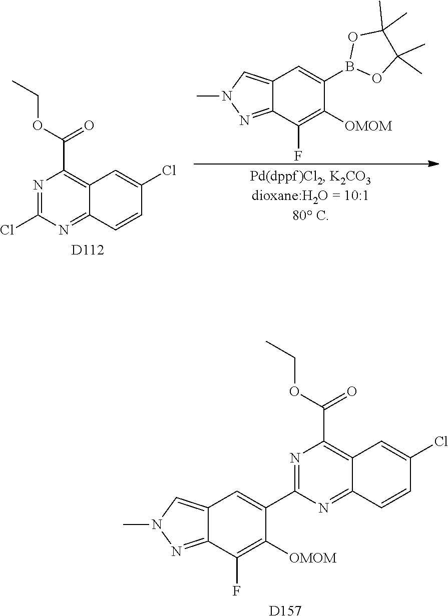

To a solution of K₃CO₄ (509.7 mg, 3.6 mmol, 2.0 equiv),7-fluoro-6-(methoxymethoxy)-2-methyl-5-(4,4,5,5-tetramethyl-1,3,2-dioxaborolan-2-yl) indazole (620.0 mg, 1.8 mmol, 1.0 equiv) and Intermediate D112 (500.0 mg, 1.8 mmol, 1.0 equiv) in dioxane (10 mL) and H₂O (1 mL) was added and Pd(dppf)Cl₂ (134.9 mg, 0.1 mmol, 0.1 equiv). After stirring for 2 h at 80° C. under a nitrogen atmosphere, the resulting mixture was concentrated under reduced pressure. The residue was purified by silica gel column chromatography and eluted with PE/EA (1:2) to afford ethyl 6-chloro-2-[7-fluoro-6-(methoxymethoxy)-2-methylindazol-5-yl]quinazoline-4-carboxylate (D157, 300.0 mg, 36.57%). LCMS: (ES, m/z): 445 [M+H]⁺.

### Synthesis of Intermediate D158

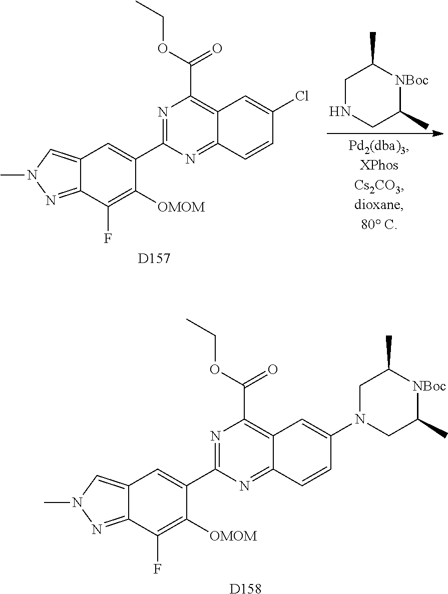

To a solution of tert-butyl (2R,6S)-2,6-dimethylpiperazine-1-carboxylate (D157, 216.7 mg, 1.0 mmol, 1.5 equiv) and Cs₂CO₃ (439.4 mg, 1.3 mmol, 2.0 equiv) in dioxane (8 mL) were added ethyl 6-chloro-2-[7-fluoro-6-(methoxymethoxy)-2-methylindazol-5-yl]quinazoline-4-carboxylate (300.0 mg, 0.6 mmol, 1.0 equiv), Pd₂(dba)₃ (61.7 mg, 0.1 mmol, 0.1 equiv) and XPhos (64.3 mg, 0.1 mmol, 0.2 equiv). After stirring for 2 h at 80° C. under a nitrogen atmosphere, the resulting mixture was concentrated under reduced pressure. The residue was purified by silica gel column chromatography and eluted with PE/EA (1:1) to afford ethyl 6-[(3R,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (D158, 360.0 mg). LCMS: (ES, m/z): 623 [M+H]⁺.

### Synthesis of Intermediate D180

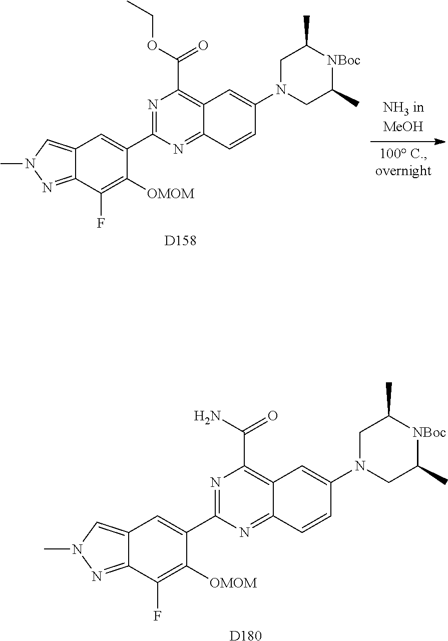

To a solution of NH₃ in MeOH (3.0 mL, 7M) was added Intermediate D158 (260.0 mg, 0.4 mmol, 1.0 equiv). After stirring overnight at 100° C. the resulting mixture was concentrated under reduced pressure. The residue was purified by silica gel column chromatography and eluted with PE/EA (1:1) to afford tert-butyl (2R,6S)-4-{4-carbamoyl-2-[7-fluoro-6-(methoxymethoxy)-2-methylindazol-5-yl]quinazolin-6-yl}-2,6-dimethylpiperazine-1-carboxylate (D180, 320.0 mg). LCMS: (ES, m/z): 594 [M+H]⁺.

### Synthesis of Intermediate D181

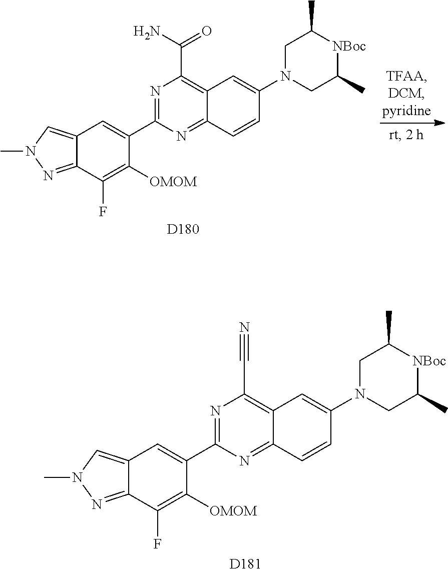

To a stirred solution of TFAA (265.3 mg, 1.2 mmol, 3.0 equiv) and TEA (170.4 mg, 1.6 mmol, 4.0 equiv) in DCM (5.0 mL) was added Intermediate D180 (250.0 mg, 0.4 mmol, 1.0 equiv) and subsequently stirred for 2 h at room temperature. The combined organic phase was washed with brine (2×5 mL), dried over anhydrous Na₂SO₄, and lastly concentrated under reduced pressure to afford tert-butyl (2S,6R)-4-(4-cyano-2-(7-fluoro-6-(methoxymethoxy)-2-methyl-2H-indazol-5-yl)quinazolin-6-yl)-2,6-dimethylpiperazine-1-carboxylate (D181, 150 mg). LCMS: (ES, m/z): 594 [M+H]⁺.

### Synthesis of Compound 707

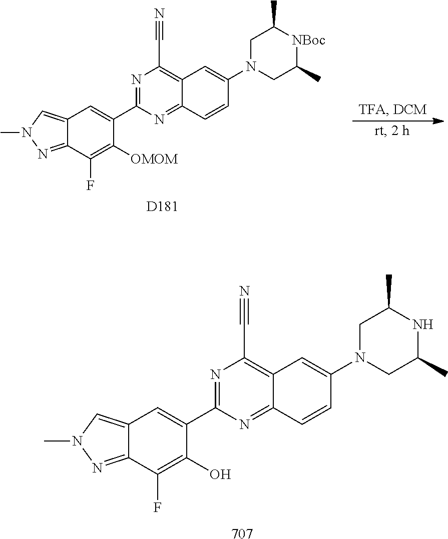

To a stirred solution of Intermediate D181 (200.0 mg, 0.3 mmol, 1.0 equiv) in DCM (2 mL) was added TFA (0.5 mL) dropwise at room temperature. The resulting mixture was stirred for 2 h and then concentrated under reduced pressure. The crude product was purified by Prep-HPLC following Condition 6, Gradient 16 as previously described to afford 6-[(3R,5S)-3,5-dimethylpiperazin-1-yl]-2-(7-fluoro-6-hydroxy-2-methylindazol-5-yl) quinazoline-4-carbonitrile (Compound 707, 20.9 mg). Absolute stereochemistry of the compound was arbitrarily assigned. LCMS: (ES, m/z): 432 [M+H]⁺. ¹H NMR: (400 MHz, DMSO-d₆) δ 12.79 (s, 1H), 8.76 (s, 1H), 8.58 (d, J=2.6 Hz, 1H), 8.21-8.11 (m, 2H), 7.12 (s, 1H), 4.18 (s, 3H), 3.93 (d, J=11.8 Hz, 2H), 2.88 (s, 2H), 2.43 (t, J=11.1 Hz, 2H), 1.10 (d, J=6.2 Hz, 6H).

### Example 256: Synthesis of Compound 712

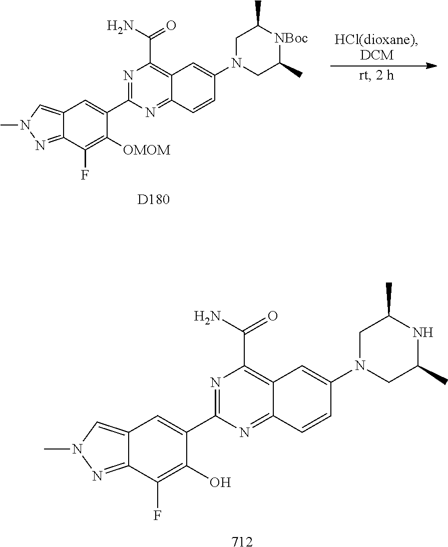

To a stirred solution of Intermediate D180 (40.0 mg, 0.067 mmol, 1.0 equiv) in DCM (2 mL) was added HCl in dioxane (0.5 mL, 4M) dropwise at room temperature. The resulting mixture was stirred for 2 h and subsequently concentrated under reduced pressure. The crude product was purified by Prep-HPLC following Condition 6, Gradient 15 as previously described to afford 6-[(3R,5S)-3,5-dimethylpiperazin-1-yl]-2-(7-fluoro-6-hydroxy-2-methylindazol-5-yl) quinazoline-4-carboxamide (Compound 712, 12.1 mg). Absolute stereochemistry of the compound was arbitrarily assigned. LCMS: (ES, m/z): 450 [M+H]⁺. ¹H NMR: (400 MHz, DMSO-d₆) δ 13.40 (s, 1H), 9.01 (s, 1H), 8.64 (s, 1H), 8.56 (d, J=2.6 Hz, 1H), 8.08 (s, 1H), 8.02-8.01 (m, 2H), 7.97 (s, 1H), 4.18 (s, 3H), 3.76 (d, J=11.2 Hz, 2H), 2.89-2.88 (s, 2H), 2.33 (t, J=11.0 Hz, 2H), 1.08 (d, J=6.2 Hz, 6H).

### Example 257: Synthesis of Compound 717

### Synthesis of Intermediate D183

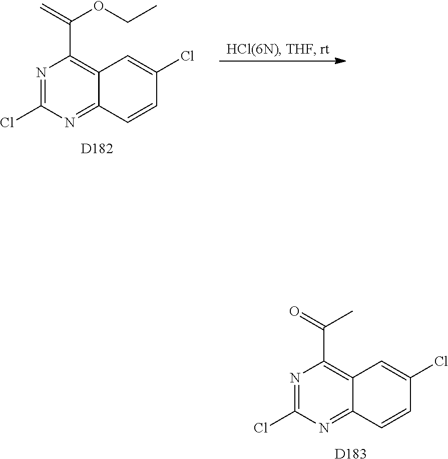

To a stirred solution of 2,6-dichloro-4-(1-ethoxyethenyl)quinazoline (D182, 600.0 mg, 2.229 mmol, 1.0 equiv) in acetone (6 mL) was added HCl (6 mL, 6N) dropwise at room temperature. The resulting mixture was stirred for 1 h at room temperature, diluted with water (20 mL) and then extracted with EtOAc (2×20 mL). The combined organic layers were washed with water (3×40 mL), brine (2×40 mL), dried over anhydrous Na₂SO₄, and the filtrate was concentrated under reduced pressure. The residue was purified by silica gel column chromatography and eluted with PE/EA (1:1) to afford 1-(2,6-dichloroquinazolin-4-yl)ethanone (D183, 370 mg). LCMS: (ES, m/z): 241 [M+H]⁺.

### Synthesis of Intermediate D184

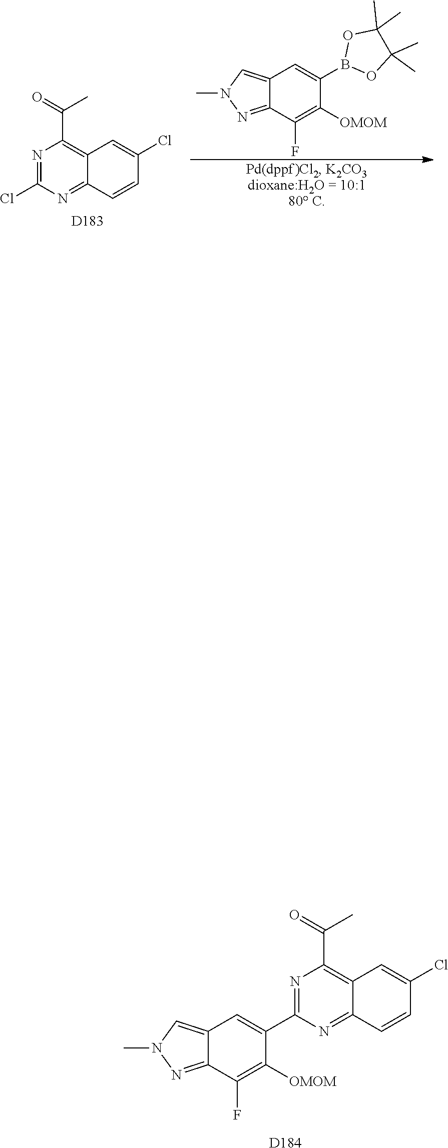

To a solution of Intermediate D183 (370.0 mg, 1.535 mmol, 1.0 equiv) and 7-fluoro-6-(methoxymethoxy)-2-methyl-5-(4,4,5,5-tetramethyl-1,3,2-dioxaborolan-2-yl) indazole (515.9 mg, 1.535 mmol, 1.0 equiv) in dioxane (5 mL) and H₂O (1 mL) were added K₃PO₄ (651.5 mg, 3.070 mmol, 2.0 equiv) and Pd(dppf)Cl₂ (112.3 mg, 0.153 mmol, 0.1 equiv). After stirring for 2 h at 80° C. under a nitrogen atmosphere, the resulting mixture was concentrated under reduced pressure. The residue was purified by silica gel column chromatography and eluted with PE/EA (1:1) to afford 1-{6-chloro-2-[7-fluoro-6-(methoxymethoxy)-2-methylindazol-5-yl]quinazolin-4-yl}ethanone (D184, 350 mg). LCMS: (ES, m/z): 415 [M+H]⁺.

### Synthesis of Intermediate D185

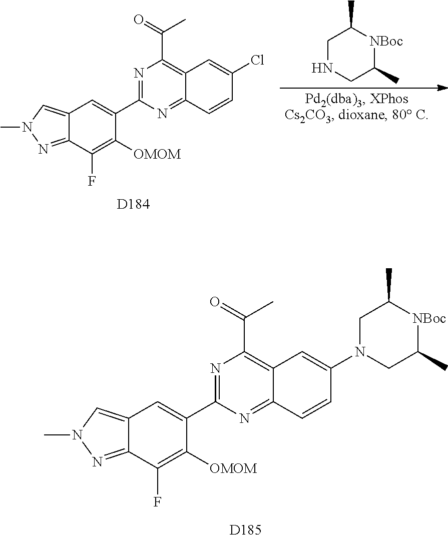

To a solution of Intermediate D184 (200.0 mg, 0.482 mmol, 1.0 equiv) and tert-butyl (2R,6S)-2,6-dimethylpiperazine-1-carboxylate (123.4 mg, 0.578 mmol, 1.2 equiv) in dioxane (5 mL) were added Cs₂CO₃ (314.2 mg, 0.964 mmol, 2.0 equiv), Pd₂(dba)₃ (44.1 mg, 0.048 mmol, 0.1 equiv), and XPhos (45.9 mg, 0.096 mmol, 0.2 equiv). After stirring for 3 h at 80° C. under a nitrogen atmosphere, the resulting mixture was concentrated under reduced pressure. The residue was purified by silica gel column chromatography and then eluted with PE/EA (1:1) to afford tert-butyl (2R,6S)-4-{4-acetyl-2-[7-fluoro-6-(methoxymethoxy)-2-methylindazol-5-yl]quinazolin-6-yl}-2,6-dimethylpiperazine-1-carboxylate (D185, 130 mg). LCMS: (ES, m/z): 593 [M+H]⁺.

### Synthesis of Compound 717

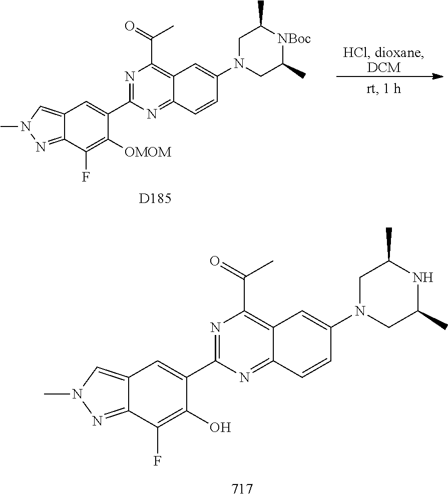

Intermediate D185 (120.0 mg, 0.202 mmol, 1.0 equiv) was added to DCM (2 mL) dropwise at room temperature. The resulting mixture was stirred for 1 h at room temperature and subsequently concentrated under reduced pressure. The crude product was purified by Prep-HPLC following Condition 8, Gradient 3 as previously described to afford 1-{6-[(3R,5S)-3,5-dimethylpiperazin-1-yl]-2-(7-fluoro-6-hydroxy-2-methylindazol-5-yl)quinazolin-4-yl}ethanone (Compound 717, 7.5 mg). Absolute stereochemistry of the compound was arbitrarily assigned. LCMS: (ES, m/z): 449 [M+H]⁺. ¹H NMR: (400 MHz, DMSO-d₆) δ 13.21 (s, 1H), 9.41 (s, 1H), 8.91 (s, 1H), 8.59 (d, J=2.6 Hz, 1H), 8.16 (d, J=9.4 Hz, 1H), 8.10 (dd, J=9.5, 2.7 Hz, 1H), 7.95 (d, J=2.6 Hz, 1H), 4.19 (s, 3H), 4.10 (d, J=12.7 Hz, 2H), 3.42-3.41 (m, 2H), 2.93-2.87 (m, 5H), 1.36 (d, J=6.5 Hz, 6H).

### Example 258: Synthesis of Compound 722

### Synthesis of Intermediate D186

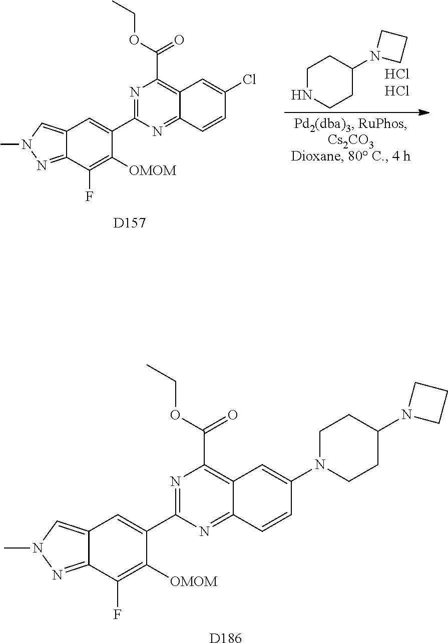

To a stirred mixture of ethyl 6-chloro-2-[7-fluoro-6-(methoxymethoxy)-2-methylindazol-5-yl]quinazoline-4-carboxylate (D157, 100 mg, 0.227 mmol, 1 equiv) and 4-(azetidin-1-yl)piperidine dihydrochloride (60 mg, 0.271 mmol, 1.19 equiv) in dioxane (2 mL) were added RuPhos (21 mg, 0.045 mmol, 0.20 equiv), Pd₂(dba)₃ (21 mg, 0.023 mmol, 0.10 equiv) and Cs₂CO₃ (148 mg, 0.454 mmol, 2.00 equiv) in portions. The resulting mixture was stirred for 4 h at 80° C. under nitrogen atmosphere and then concentrated under reduced pressure. The residue was purified by silica gel column chromatography and eluted with PE/EA (1:1) to afford ethyl 6-[4-(azetidin-1-yl)piperidin-1-yl]-2-[7-fluoro-6-(methoxymethoxy)-2-methylindazol-5-yl]quinazoline-4-carboxylate (D186, 95 mg). LCMS: (ES, m/z): 549 [M+H]⁺.

### Synthesis of Intermediate D187

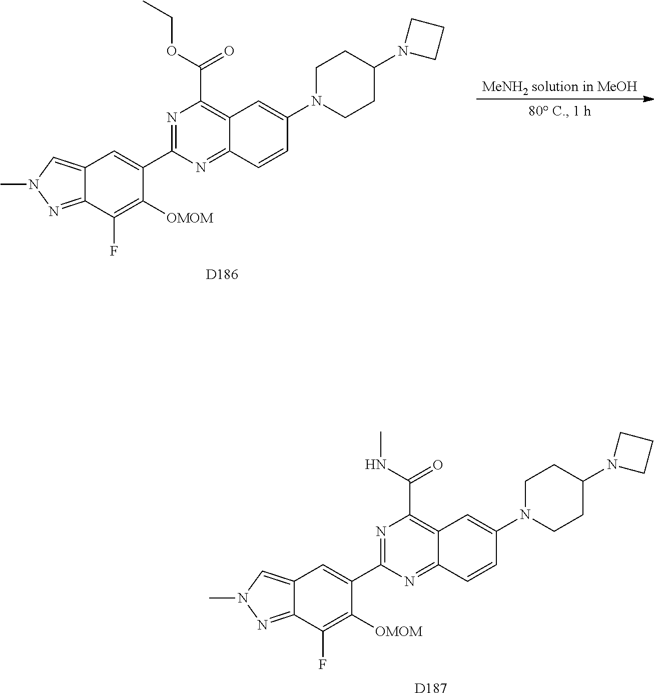

A mixture of Intermediate D186 (95 mg, 0.174 mmol, 1 equiv) and 2M MeNH₃ in MeOH (1 mL) was stirred for 2 h at 80° C. and subsequently concentrated under reduced pressure to afford 6-[4-(azetidin-1-yl)piperidin-1-yl]-2-[7-fluoro-6-(methoxymethoxy)-2-methylindazol-5-yl]-N-methylquinazoline-4-carboxamide (D187, 90 mg). LCMS: (ES, m/z): 534 [M+H]⁺.

### Synthesis of Compound 722

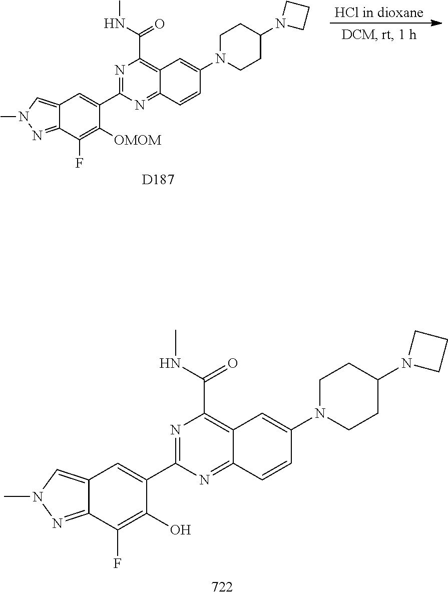

A solution of Intermediate D187 (90 mg, 0.170 mmol, 1 equiv) in DCM (0.8 mL) was treated with 4 M HCl (gas) in 1,4-dioxane (0.4 mL) for 1 h at room temperature and then concentrated under reduced pressure. The crude product was purified by reversed-phase flash chromatography following Condition 9, Gradient 5 as previously described to afford 6-[4-(azetidin-1-yl)piperidin-1-yl]-2-(7-fluoro-6-hydroxy-2-methylindazol-5-yl)-N-methylquinazoline-4-carboxamide (Compound 722, 55.0 mg). LCMS: (ES, m/z): 490 [M+H]⁺. ¹H NMR: (400 MHz, DMSO-d₆) δ 13.42 (s, 1H), 9.21 (d, J=5.0 Hz, 1H), 9.04 (s, 1H), 8.57 (d, J=2.6 Hz, 1H), 8.16 (d, J=2.2 Hz, 1H), 7.99 (d, J=3.1 Hz, 2H), 4.19 (s, 3H), 3.75 (d, J=12.3 Hz, 3H), 3.17 (s, 3H), 3.05 (t, J=10.8 Hz, 2H), 2.96 (d, J=4.6 Hz, 3H), 2.29 (s, 1H), 1.97 (d, J=7.2 Hz, 2H), 1.78 (d, J=12.5 Hz, 2H), 1.32 (q, J=11.3, 9.1 Hz, 2H).

### Example 259: Synthesis of Compound 728

### Synthesis of Intermediate D188

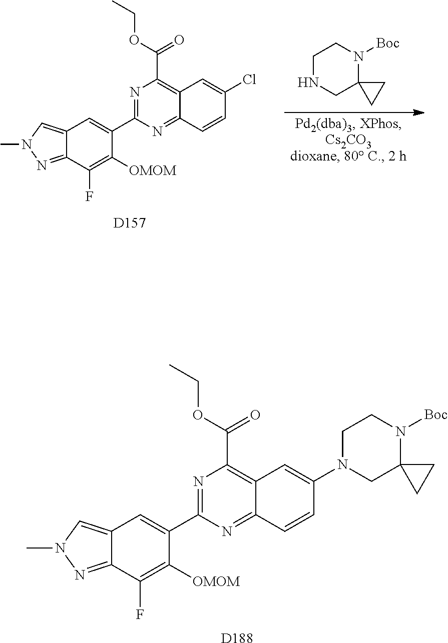

To a solution of Cs₂CO₃ (322.2 mg, 0.990 mmol, 2.0 equiv), tert-butyl 4,7-diazaspiro[2.5]octane-4-carboxylate (157.4 mg, 0.742 mmol, 1.5 equiv) and Intermediate D157 (220.0 mg, 0.495 mmol, 1.0 equiv) in dioxane (3.5 mL) were added XPhos (47.1 mg, 0.099 mmol, 0.2 equiv) and Pd₂(dba)₃ (45.2 mg, 0.050 mmol, 0.1 equiv). After stirring for 2 h at 80° C. under a nitrogen atmosphere, the resulting mixture was concentrated under reduced pressure. The residue was purified by silica gel column chromatography and eluted with PE/EA (1:2) to afford ethyl 6-[4-(tert-butoxycarbonyl)-4,7-diazaspiro [2.5]octan-7-yl]-2-[7-fluoro-6-(methoxymethoxy)-2-methylindazol-5-yl]quinazoline-4-carboxylate (D188, 210.0 mg). LCMS: (ES, m/z): 621 [M+H]⁺.

### Synthesis of Intermediate D189

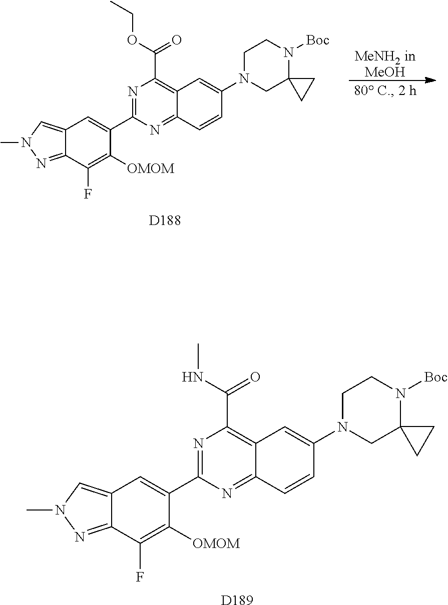

To a solution of MeNH₂ (0.5 mL) in MeOH was added Intermediate D188 (210.0 mg, 0.338 mmol, 1.0 equiv). After stirring for 2 h at 80° C., the resulting mixture was concentrated under reduced pressure. The residue was purified by silica gel column chromatography and eluted with PE/EA (1:1) to afford tert-butyl 7-{2-[7-fluoro-6-(methoxymethoxy)-2-methylindazol-5-yl]-4-(methylcarbamoyl)quinazolin-6-yl}-4,7-diazaspiro[2.5]octane-4-carboxylate (D189, 180.0 mg) as a light yellow solid. LCMS: (ES, m/z): 606 [M+H]⁺.

### Synthesis of Compound 728

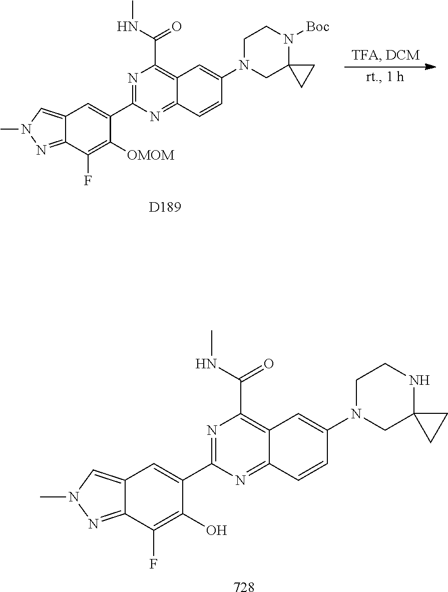

Intermediate D189 (200.0 mg, 0.330 mmol, 1.0 equiv) in DCM (2 mL) and TFA (0.5 mL), stirred for 2 h at room temperature, and then concentrated under reduced pressure. The crude product was purified by Prep-HPLC following Condition 6, Gradient 14 as previously described to afford 6-{4,7-diazaspiro[2.5]octan-7-yl}-2-(7-fluoro-6-hydroxy-2-methylindazol-5-yl)-N-methylquinazoline-4-carboxamide (Compound 728, 19.7 mg). LCMS: (ES, m/z): 462 [M+H]0.1H NMR: (400 MHz, DMSO-d₆) δ 9.02 (s, 1H), 8.54 (d, J=2.6 Hz, 1H), 8.08 (d, J=2.6 Hz, 1H), 8.03-7.91 (m, 2H), 4.17 (s, 3H), 3.31 (t, J=5.1 Hz, 2H), 3.20 (s, 2H), 2.95-2.93 (m, 5H), 0.62-0.50 (m, 4H).

### Example 260: Synthesis of Compound 729

### Synthesis of Intermediate D190

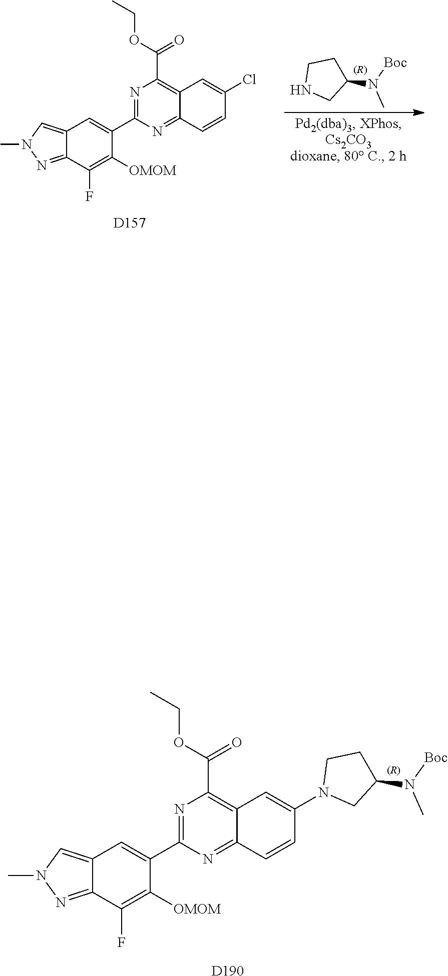

To a mixture of Cs₂CO₃ (322.2 mg, 0.990 mmol, 2.0 equiv), tert-butyl N-methyl-N-[(3R)-pyrrolidin-3-yl]carbamate (148.5 mg, 0.742 mmol, 1.5 equiv) and Intermediate D157 (220.0 mg, 0.495 mmol, 1.0 equiv) in dioxane (3.5 mL) were added XPhos (47.1 mg, 0.099 mmol, 0.2 equiv) and Pd₂(dba)₃ (45.29 mg, 0.050 mmol, 0.1 equiv). After stirring for 2 h at 80° C. under a nitrogen atmosphere, the resulting mixture was concentrated under reduced pressure. The residue was purified by silica gel column chromatography and eluted with PE/EA (1:2) to afford ethyl 6-[(3R)-3-[(tert-butoxycarbonyl)(methyl)amino]pyrrolidin-1-yl]-2-[7-fluoro-6-(methoxymethoxy)-2-methylindazol-5-yl]quinazoline-4-carboxylate (D190, 220.0 mg, 73.09%) as a yellow solid. LCMS: (ES, m/z): 609 [M+H]⁺.

### Synthesis of Intermediate D191

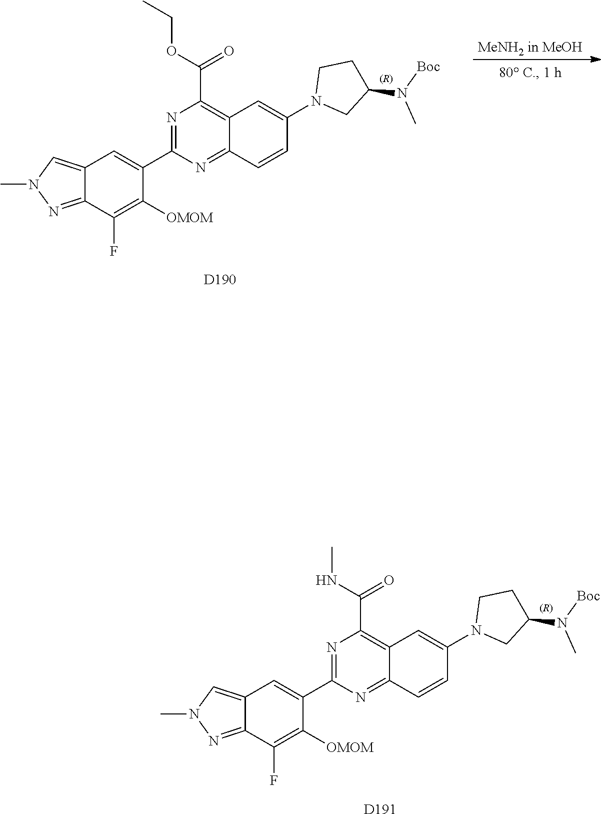

To a solution of MeNH₂ (0.5 mL, 7M in MeOH) was added Intermediate D190 (220.0 mg, 0.361 mmol, 1.0 equiv). After stirring for 2 h at 80° C., the resulting mixture was concentrated under reduced pressure. The residue was purified by silica gel column chromatography and eluted with PE/EA (1:1) to afford tert-butyl N-[(3R)-1-{2-[7-fluoro-6-(methoxymethoxy)-2-methylindazol-5-yl]-4-(methylcarbamoyl)quinazolin-6-yl}pyrrolidin-3-yl]-N-methylcarbamate (D191, 175.0 mg, 81.56%). LCMS: (ES, m/z): 594 [M+H]⁺.

### Synthesis of Compound 729

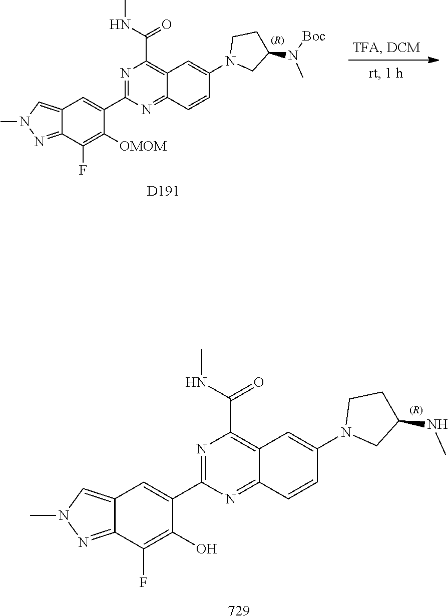

To a stirred solution of Intermediate D191 (200.0 mg, 0.337 mmol, 1.0 equiv) in DCM (2 mL) was added TFA (0.5 mL) dropwise at room temperature. The resulting mixture was stirred for 2 h and subsequently concentrated under reduced pressure. The crude product was purified by Prep-HPLC following Condition 6, Gradient 13 as previously described to afford 2-(7-fluoro-6-hydroxy-2-methylindazol-5-yl)-N-methyl-6-[(3R)-3-(methylamino)pyrrolidin-1-yl]quinazoline-4-carboxamide (Compound 729, 20.9 mg). Absolute stereochemistry of the compound was arbitrarily assigned. LCMS: (ES, m/z): 450 [M+H]⁺. ¹H NMR: (400 MHz, DMSO-d₆) δ 13.51 (s, 1H), 9.16 (d, J=5.1 Hz, 1H), 8.99 (s, 1H), 8.55 (d, J=2.6 Hz, 1H), 8.00 (d, J=9.3 Hz, 1H), 7.75 (d, J=2.7 Hz, 1H), 7.60 (dd, J=9.3, 2.7 Hz, 1H), 4.18 (s, 3H), 3.62-3.32 (m, 4H), 3.20 (dd, J=9.9, 4.4 Hz, 1H), 2.96 (d, J=4.7 Hz, 3H), 2.35 (s, 3H), 2.17 (dq, J=12.9, 6.7 Hz, 1H), 1.91 (q, J=6.4 Hz, 1H).

### Example 261: Synthesis of Compound 732

### Synthesis of Intermediate D192

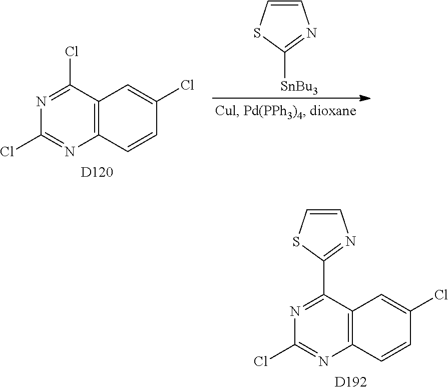

To a stirred solution of 2,4,6-trichloroquinazoline (D120, 420 mg, 1.799 mmol, 1 equiv) and 2-(tributylstannyl)-1,3-thiazole (673 mg, 1.799 mmol, 1 equiv) in 1,4-dioxane (8 mL) were added CuI (68 mg, 0.360 mmol, 0.2 equiv) and Pd(PPh₃)₄(207 mg, 0.180 mmol, 0.1 equiv) at room temperature under nitrogen atmosphere. The resulting mixture was stirred for 3 h, allowed to cool to room temperature, and then concentrated under reduced pressure. The residue was purified by silica gel column chromatography and eluted with PE/EA (1:1) to afford 2,6-dichloro-4-(1,3-thiazol-2-yl)quinazoline (D192, 420 mg). LCMS: (ES, m/z): 283 [M+H]⁺.

### Synthesis of Intermediate D193

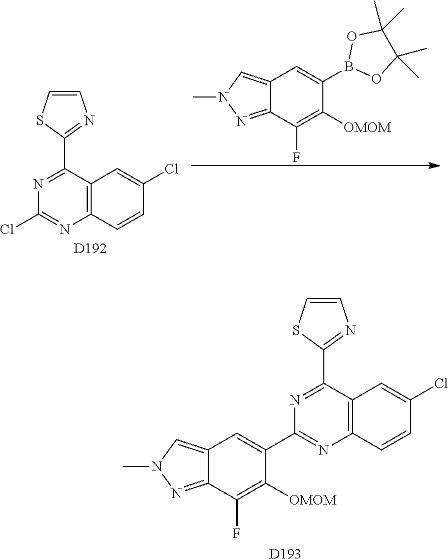

To a stirred mixture of Intermediate D192 (420 mg, 1.489 mmol, 1 equiv) and 7-fluoro-6-(methoxymethoxy)-2-methyl-5-(4,4,5,5-tetramethyl-1,3,2-dioxaborolan-2-yl) indazole (550 mg, 1.638 mmol, 1.1 equiv) in 1,4-dioxane (10 mL) and H₂O (1 mL) were added K₃PO₄ (947 mg, 4.467 mmol, 3 equiv) and Pd(dppf)Cl₂·CH₂Cl₂ (121 mg, 0.149 mmol, 0.1 equiv) at room temperature under nitrogen atmosphere. The resulting mixture was stirred for 3 h at room temperature. The resulting mixture was stirred for 2 h at 70° C. under nitrogen atmosphere, allowed to cool to room temperature, and subsequently concentrated under reduced pressure. The residue was purified by silica gel column chromatography and eluted with PE/EA (1:1) to afford 6-chloro-2-[7-fluoro-6-(methoxymethoxy)-2-methylindazol-5-yl]-4-(1,3-thiazol-2-yl)quinazoline (D193, 300 mg). LCMS: (ES, m/z): 457 [M+H]⁺.

### Synthesis of Intermediate D194

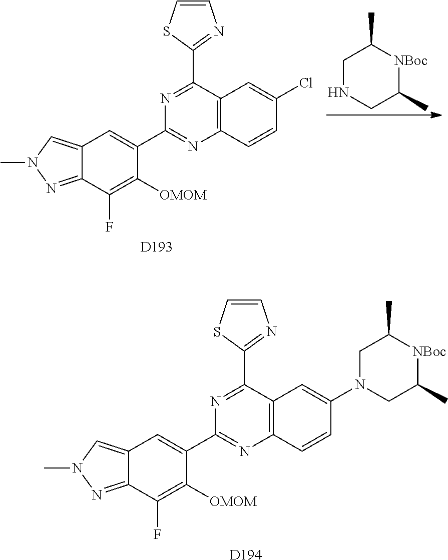

To a stirred mixture of Intermediate D193 (300 mg, 0.658 mmol, 1 equiv) and tert-butyl (2R,6S)-2,6-dimethylpiperazine-1-carboxylate (211 mg, 0.987 mmol, 1.5 equiv) in 1,4-dioxane (3 mL) were added Cs₂CO₃ (643 mg, 1.974 mmol, 3 equiv), RuPhos (61 mg, 0.132 mmol, 0.2 equiv) and Pd₂(dba)₃ (60 mg, 0.066 mmol, 0.1 equiv) at room temperature under nitrogen atmosphere. The resulting mixture was stirred for 3 h at 100° C., allowed to cool to room temperature, and subsequently concentrated under reduced pressure. The residue was purified by silica gel column chromatography and eluted with PE/EA (1:1) to afford tert-butyl (2R,6S)-4-{2-[7-fluoro-6-(methoxymethoxy)-2-methylindazol-5-yl]-4-(1,3-thiazol-2-yl)quinazolin-6-yl}-2,6-dimethylpiperazine-1-carboxylate (D194, 200 mg). LCMS: (ES, m/z): 634 [M+H]⁺.

### Synthesis of Compound 732

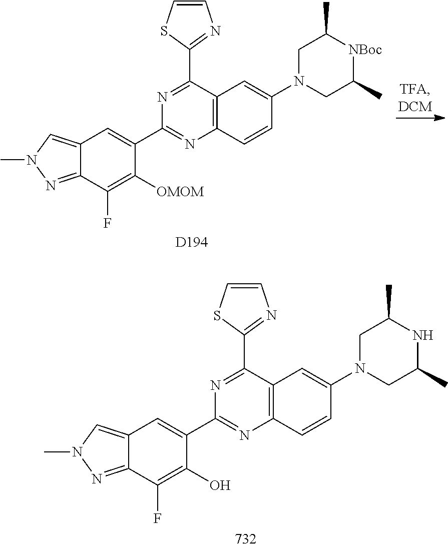

To a stirred solution of Intermediate D194 (200 mg, 0.316 mmol, 1 equiv) in DCM (3 mL) was added TFA (1 mL) at room temperature. The resulting mixture was stirred for 2 h at room temperature and then concentrated under reduced pressure. The residue was purified by reversed-phase flash chromatography following Condition 10, Gradient 10 as previously described to afford 5-{6-[(3R,5S)-3,5-dimethylpiperazin-1-yl]-4-(1,3-thiazol-2-yl)quinazolin-2-yl}-7-fluoro-2-methylindazol-6-ol (Compound 732, 50 mg). Absolute stereochemistry of the compound was arbitrarily assigned. LCMS: (ES, m/z): 490 [M+H]⁺. ¹H NMR: (300 MHz, DMSO-d₆) δ 13.46 (s, 1H), 8.94 (s, 1H), 8.81 (d, J=1.0 Hz, 1H), 8.61 (d, J=2.6 Hz, 1H), 8.34 (d, J=3.2 Hz, 1H), 8.20 (d, J=3.2 Hz, 1H), 8.01 (d, J=2.6 Hz, 2H), 4.19 (s, 3H), 3.84 (d, J=11.4 Hz, 2H), 2.91 (s, 2H), 2.40 (t, J=11.1 Hz, 2H), 1.10 (d, J=6.2 Hz, 6H).

### Example 262: Synthesis of Compound 737

### Synthesis of Intermediate D158

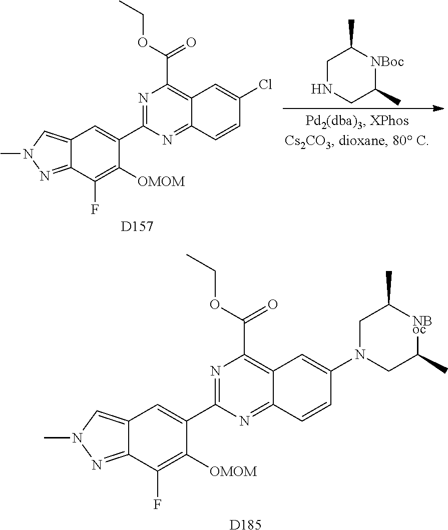

To a stirred solution of Intermediate D157 (200 mg, 0.450 mmol, 1 equiv) and tert-butyl (2R,6S)-2,6-dimethylpiperazine-1-carboxylate (144 mg, 0.675 mmol, 1.5 equiv) in 1,4-dioxane were added XPhos (42.87 mg, 0.090 mmol, 0.2 equiv), Cs₂CO₃ (292 mg, 0.900 mmol, 2 equiv) and Pd₂(dba)₃ (41 mg, 0.045 mmol, 0.1 equiv) at room temperature under nitrogen atmosphere. The resulting mixture was stirred for 3 h at 80° C., quenched with water (30 mL) at room temperature, and subsequently extracted with EtOAc (3×20 mL). The combined organic layers were washed with brine (3×10 mL), dried over anhydrous Na₂SO₄, and the filtrate was concentrated under reduced pressure. The residue was purified by silica gel column chromatography and eluted with CH₂Cl₂/MeOH (10:1) to afford ethyl 6-[(3R,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (D158, 130 mg). LCMS: (ES, m/z): 623 [M+H]⁺.

### Synthesis of Intermediate D195

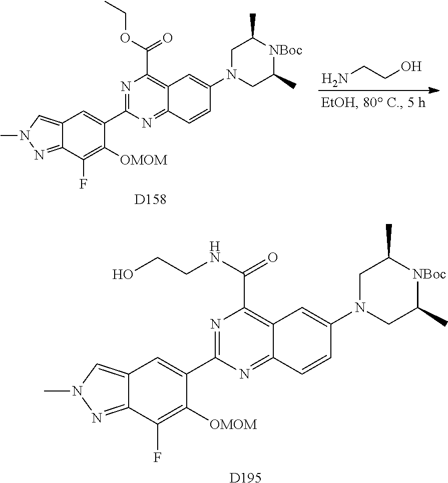

To a stirred solution of Intermediate D158 (200 mg, 0.321 mmol, 1 equiv) in EtOH (5 mL) was added 2-aminoethan-1-ol (15 mL) at room temperature. The resulting mixture was stirred for 5 h at 80° C., quenched with water at room temperature, and then extracted with EtOAc (3×10 mL). The combined organic layers were washed with brine (3×10 mL), dried over anhydrous Na₂SO₄, and the filtrate was concentrated under reduced pressure. The resulting solid was dried under infrared light to afford tert-butyl (2R,6S)-4-{2-[7-fluoro-6-(methoxymethoxy)-2-methylindazol-5-yl]-4-[(2-hydroxyethyl)carbamoyl]quinazolin-6-yl}-2,6-dimethylpiperazine-1-carboxylate (D195, 180 mg). LCMS: (ES, m/z): 638 [M+H]⁺.

### Synthesis of Compound 737

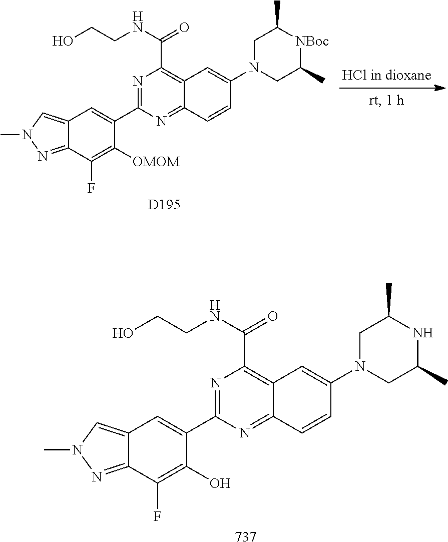

A mixture of Intermediate D195 (120 mg, 0.188 mmol, 1 equiv) and 4M HCl (gas) in 1,4-dioxane (2 mL) was stirred for 1 h at room temperature and subsequently concentrated under reduced pressure. The residue was purified by reversed-phase flash chromatography following Condition 13, Gradient 1 as previously described to afford 6-[(3R,5S)-3,5-dimethylpiperazin-1-yl]-2-(7-fluoro-6-hydroxy-2-methylindazol-5-yl)-N-(2-hydroxyethyl)quinazoline-4-carboxamide (Compound 737, 60 mg). Absolute stereochemistry of the compound was arbitrarily assigned. LCMS: (ES, m/z): 494 [M+H]⁺. ¹H NMR: (400 MHz, DMSO-d₆) δ 13.43 (s, 1H), 9.18 (t, J=5.8 Hz, 1H), 8.99 (s, 1H), 8.58 (d, J=2.6 Hz, 1H), 8.07-8.00 (m, 3H), 4.86 (t, J=5.5 Hz, 1H), 4.19 (s, 3H), 3.80 (d, J=11.6 Hz, 2H), 3.65 (q, J=5.9 Hz, 2H), 3.52 (q, J=6.0 Hz, 2H), 2.91 (d, J=9.8 Hz, 2H), 2.36 (t, J=11.1 Hz, 2H), 1.10 (d, J=6.2 Hz, 6H).

### Example 263: Synthesis of Compound 742

### Synthesis of Intermediate D196

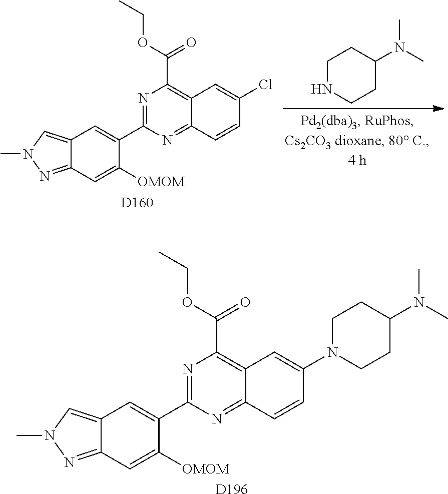

To a stirred mixture of ethyl 6-chloro-2-[6-(methoxymethoxy)-2-methylindazol-5-yl]quinazoline-4-carboxylate (D160, 120 mg, 0.281 mmol, 1 equiv) and N,N-dimethylpiperidin-4-amine (43 mg, 0.335 mmol, 1.20 equiv) in dioxane (2 mL) were added RuPhos (26 mg, 0.056 mmol, 0.20 equiv), Pd₂(dba)₃ (26 mg, 0.028 mmol, 0.10 equiv) and Cs₂CO₃ (183 mg, 0.562 mmol, 2.00 equiv) at room temperature. The resulting mixture was stirred for 4 h at 80° C. under nitrogen atmosphere and later concentrated under reduced pressure. The residue was purified by silica gel column chromatography and eluted with PE/EA (1:1) to afford ethyl 6-[4-(dimethylamino)piperidin-1-yl]-2-[6-(methoxymethoxy)-2-methylindazol-5-yl]quinazoline-4-carboxylate (D196, 110 mg). LCMS: (ES, m/z): 519 [M+H]⁺.

### Synthesis of Intermediate D197

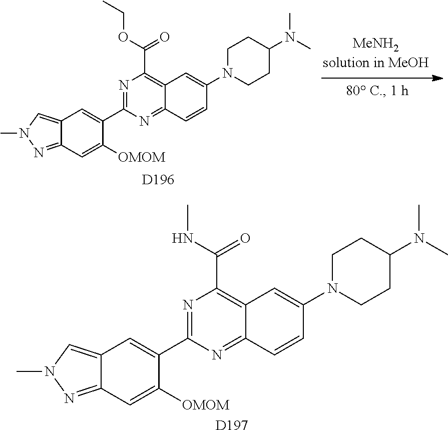

A mixture of Intermediate D196 (100 mg, 0.193 mmol, 1 equiv) and 2M MeNH₃ in MeOH (2 mL) was stirred for 2 h at 80° C. and subsequently concentrated under reduced pressure to afford 6-[4-(dimethylamino)piperidin-1-yl]-2-[6-(methoxymethoxy)-2-methylindazol-5-yl]-N-methylquinazoline-4-carboxamide (D197, 90 mg). LCMS: (ES, m/z): 504 [M+H]⁺.

### Synthesis of Compound 742

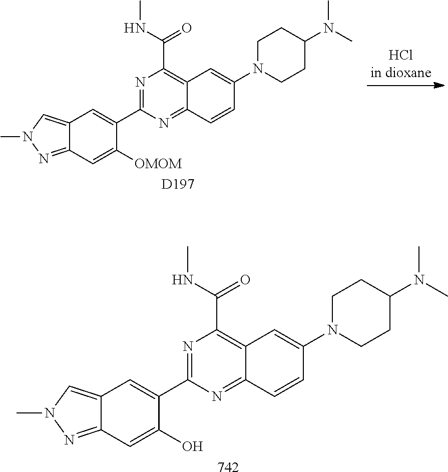

A solution of Intermediate D197 (90 mg, 0.179 mmol, 1 equiv) in DCM (2 mL) was treated with 4 M HCl (gas) in 1,4-dioxane (0.5 mL) for 1 h at room temperature and then concentrated under reduced pressure. The crude product was purified by reversed-phase flash chromatography following Condition 9, Gradient 5 as previously described to afford 6-[4-(dimethylamino)piperidin-1-yl]-2-(6-hydroxy-2-methylindazol-5-yl)-N-methylquinazoline-4-carboxamide (Compound 742, 45.5 mg). LCMS: (ES, m/z): 460 [M+H]⁺. ¹H NMR: (400 MHz, DMSO-d₆) δ 13.21 (s, 1H), 9.21 (d, J=9.2 Hz, 2H), 8.44 (s, 1H), 8.16 (s, 1H), 7.97 (d, J=2.0 Hz, 2H), 6.91 (s, 1H), 4.14 (s, 3H), 3.93 (d, J=12.8 Hz, 2H), 2.97 (d, J=4.9 Hz, 3H), 2.91 (d, J=11.3 Hz, 2H), 2.31 (d, J=10.9 Hz, 1H), 2.22 (s, 6H), 1.91 (d, J=12.5 Hz, 2H), 1.53 (q, J=10.1, 9.5 Hz, 2H).

### Example 264: Synthesis of Compound 668

### Synthesis of Intermediate D198

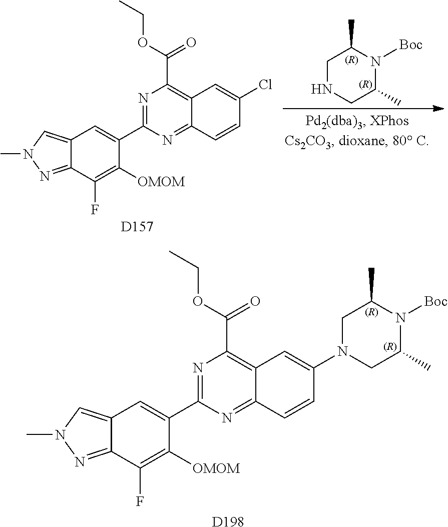

To a stirred mixture of ethyl 6-chloro-2-[7-fluoro-6-(methoxymethoxy)-2-methylindazol-5-yl]quinazoline-4-carboxylate (D157, 120 mg, 0.270 mmol, 1 equiv) and tert-butyl (2R,6R)-2,6-dimethylpiperazine-1-carboxylate (69 mg, 0.322 mmol, 1.20 equiv) in dioxane (4 mL) were added RuPhos (25 mg, 0.054 mmol, 0.20 equiv), Pd₂(dba)₃ (25 mg, 0.027 mmol, 0.10 equiv) and Cs₂CO₃ (176 mg, 0.540 mmol, 2.00 equiv) at room temperature. The resulting mixture was stirred for 4 h at 80° C. under nitrogen atmosphere and subsequently concentrated under reduced pressure. The residue was purified by silica gel column chromatography and eluted with PE/EA (1:1) to afford ethyl 6-[(3R,5R)-4-(tert-butoxycarbonyl)-3,5-dimethylpiperazin-1-yl]-2-[7-fluoro-6-(methoxymethoxy)-2-methylindazol-5-yl]quinazoline-4-carboxylate (D198, 100 mg). LCMS: (ES, m/z): 623 [M+H]⁺.

### Synthesis of Intermediate D199

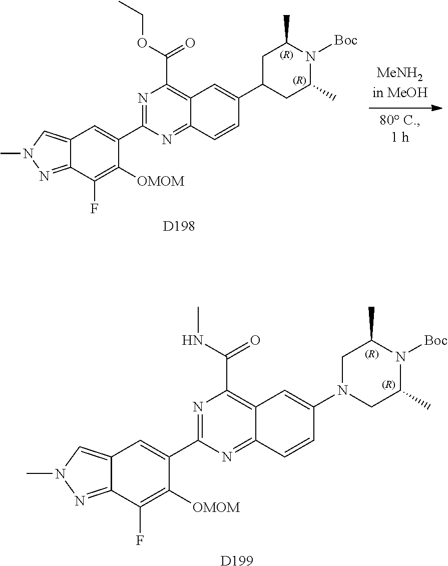

A mixture of Intermediate D198 (100 mg, 0.161 mmol, 1 equiv) and 2M MeNH₃ in MeOH (2.0 mL) was stirred for 2 h at 80° C. and subsequently concentrated under reduced pressure to afford tert-butyl (2R,6R)-4-{2-[7-fluoro-6-(methoxymethoxy)-2-methylindazol-5-yl]-4-(methylcarbamoyl)quinazolin-6-yl}-2,6-dimethylpiperazine-1-carboxylate (D199, 80 mg). LCMS: (ES, m/z): 608 [M+H]⁺.

### Synthesis of Compound 668

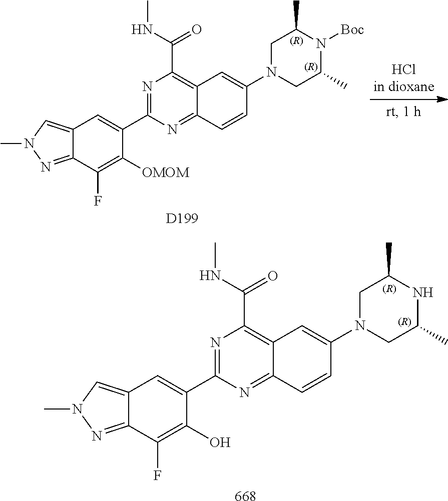

A solution of Intermediate D199 (50 mg, 0.082 mmol, 1 equiv) in DCM (2.0 mL) was treated with 4M HCl (gas) in 1,4-dioxane (0.4 mL) for 1 h at room temperature and then concentrated under reduced pressure. The crude product was purified by reversed-phase flash chromatography following Condition 9, Gradient 5 as previously described to afford 6-[(3R,5R)-3,5-dimethylpiperazin-1-yl]-2-(7-fluoro-6-hydroxy-2-methylindazol-5-yl)-N-methylquinazoline-4-carboxamide (Compound 668, 35.5 mg). Absolute stereochemistry of the compound was arbitrarily assigned. LCMS: (ES, m/z): 464 [M+H]⁺. ¹H NMR: (400 MHz, DMSO-d₆) δ 13.43 (s, 1H), 9.21 (d, J=5.1 Hz, 1H), 9.04 (d, J=1.0 Hz, 1H), 8.56 (d, J=2.6 Hz, 1H), 8.14 (d, J=2.5 Hz, 1H), 8.06-7.93 (m, 2H), 4.19 (s, 3H), 3.38 (dd, J=11.7, 3.2 Hz, 2H), 3.25 (d, J=3.5 Hz, 2H), 3.02 (dd, J=11.7, 6.3 Hz, 2H), 2.97 (d, J=4.8 Hz, 3H), 1.13 (d, J=6.4 Hz, 6H).

### Example 265: Synthesis of Compound 653

### Synthesis of Intermediate D200

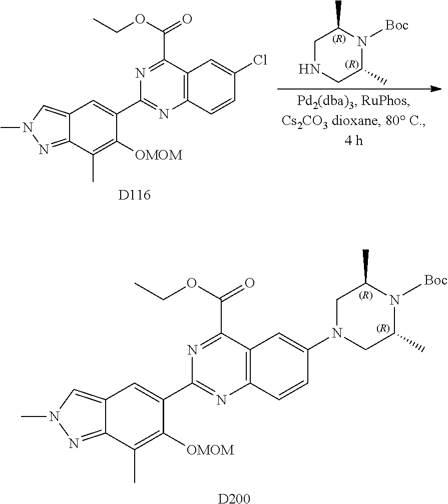

To a stirred mixture of Intermediate D116 (130 mg, 0.295 mmol, 1.0 equiv) and tert-butyl (2R,6R)-2,6-dimethylpiperazine-1-carboxylate (76 mg, 0.355 mmol, 1.2 equiv) in dioxane (3 mL) were added RuPhos (28 mg, 0.060 mmol, 0.2 equiv), Pd₂(dba)₃ (27.00 mg, 0.029 mmol, 0.1 equiv) and Cs₂CO₃ (192 mg, 0.589 mmol, 2.0 equiv) in portions. The resulting mixture was stirred for 5 h at 80° C. under nitrogen atmosphere and then concentrated under reduced pressure. The residue was purified by silica gel column chromatography and eluted with PE/EA (1:1) to afford ethyl ethyl 6-[(3R,5R)-4-(tert-butoxycarbonyl)-3,5-dimethylpiperazin-1-yl]-2-[6-(methoxymethoxy)-2,7-dimethylindazol-5-yl]quinazoline-4-carboxylate (D200, 100 mg). LCMS: (ES, m/z): 619 [M+H]⁺.

### Synthesis of Intermediate D201

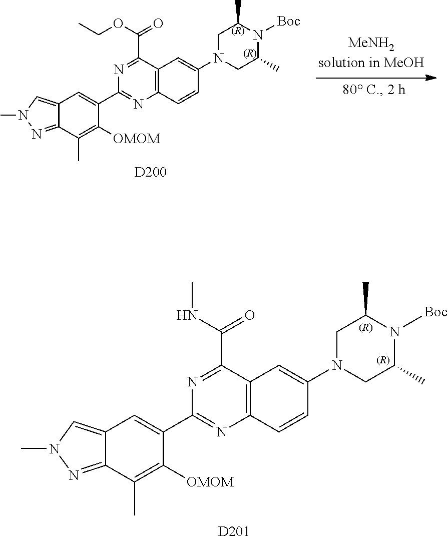

A mixture of Intermediate D200 (100 mg, 0.162 mmol, 1 equiv) and 2M MeNH₃ in MeOH (5 mL) was stirred for 2 h at 80° C. and then concentrated under reduced pressure to afford tert-butyl (2R,6R)-4-{2-[6-(methoxymethoxy)-2,7-dimethylindazol-5-yl]-4-(methylcarbamoyl)quinazolin-6-yl}-2,6-dimethylpiperazine-1-carboxylate (D201, 80 mg, crude product). LCMS: (ES, m/z): 604 [M+H]⁺.

### Synthesis of Compound 653

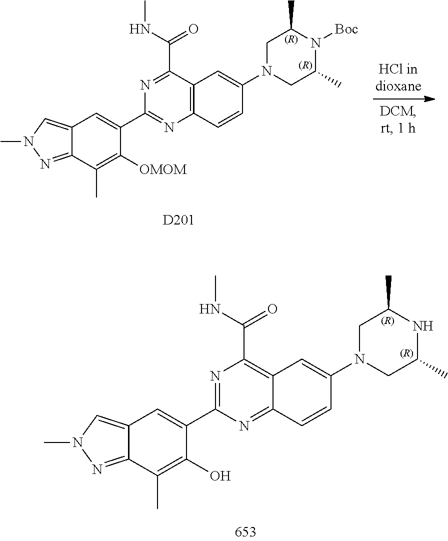

A solution of Intermediate D201 (80 mg, 0.133 mmol, 1 equiv) in DCM (0.8 mL) was treated with HCl (gas) in 1,4-dioxane (0.4 mL) for 1 h at room temperature and subsequently concentrated under reduced pressure. The crude product was purified by reversed-phase flash chromatography following Condition 9, Gradient 5 as previously described to afford 6-[(3R,5R)-3,5-dimethylpiperazin-1-yl]-2-(6-hydroxy-2,7-dimethylindazol-5-yl)-N-methylquinazoline-4-carboxamide (Compound 653, 23.6 mg). Absolute stereochemistry of the compound was arbitrarily assigned. LCMS: (ES, m/z): 460 [M+H]⁺. RT=1.361 min on chiral-SFC. ¹H NMR: (400 MHz, DMSO-d₆) δ 13.40 (s, 1H), 9.18 (d, J=5.1 Hz, 1H), 9.08 (s, 1H), 8.41 (s, 1H), 8.11 (d, J=2.3 Hz, 1H), 8.01-7.91 (m, 2H), 4.16 (s, 3H), 3.36 (dd, J=11.7, 3.4 Hz, 2H), 3.26 (s, 2H), 3.05-2.98 (m, 2H), 2.97 (d, J=4.8 Hz, 3H), 2.41 (s, 3H), 1.14 (d, J=6.5 Hz, 6H).

### Example 266: Synthesis of Compound 743

### Synthesis of Intermediate D202

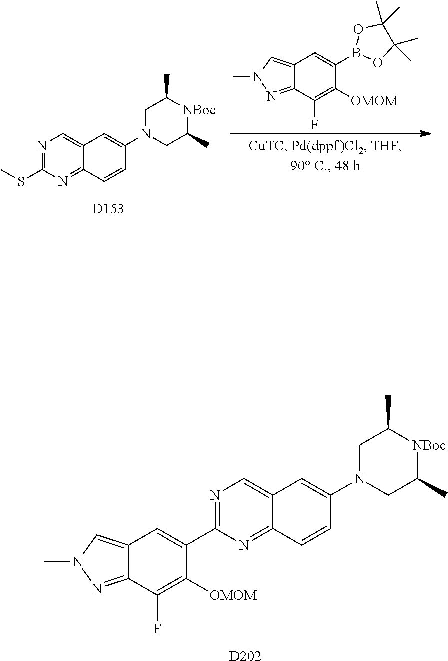

To a stirred solution of tert-butyl (2R,6S)-2,6-dimethyl-4-[2-(methylsulfanyl)quinazolin-6-yl]piperazine-1-carboxylate (D153, 750 mg, 1.93 mmol, 1 equiv) and 7-fluoro-6-(methoxymethoxy)-2-methyl-5-(4,4,5,5-tetramethyl-1,3,2-dioxaborolan-2-yl) indazole (974 mg, 2.895 mmol, 1.5 equiv) in THF (20 mL) were added CuTC (810 mg, 4.246 mmol, 2.2 equiv) and Pd(dppf)Cl₂ (142 mg, 0.193 mmol, 0.1 equiv) at room temperature under nitrogen atmosphere. The resulting mixture was stirred for 48 h at 90° C. under nitrogen atmosphere, allowed to cool to room temperature, quenched by the addition of water (100 mL) at room temperature, and then extracted with EtOAc (3×30 mL). The combined organic layers were washed with brine (1×50 mL), dried over anhydrous Na₂SO₄, and the filtrate was concentrated under reduced pressure. The residue was purified by reversed-phase flash chromatography following Condition 10, Gradient 11 as previously described to afford tert-butyl (2R,6S)-4-{2-[7-fluoro-6-(methoxymethoxy)-2-methylindazol-5-yl]quinazolin-6-yl}-2,6-dimethylpiperazine-1-carboxylate (D202, 300 mg). LCMS: (ES, m/z): 551 [M+H]⁺.

### Synthesis of Compound 743

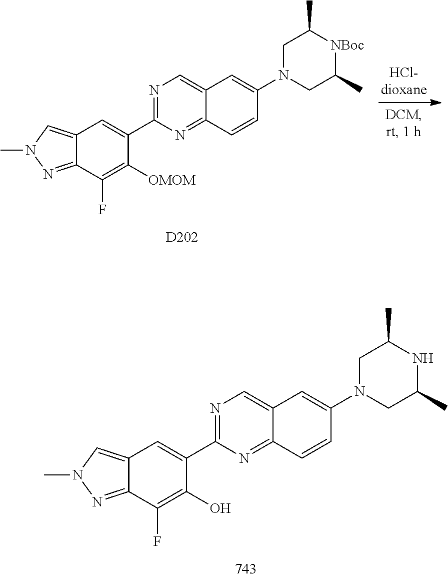

A solution of Intermediate D202 (320 mg, 0.581 mmol, 1 equiv) and HCl (gas) in 1,4-dioxane (0.7 mL) in DCM (2 mL) was stirred for 1 h at room temperature under nitrogen atmosphere and subsequently concentrated under reduced pressure. The residue was purified by reversed-phase flash chromatography following Condition 10, Gradient 17 as previously described to afford 5-{6-[(3R,5S)-3,5-dimethylpiperazin-1-yl]quinazolin-2-yl}-7-fluoro-2-methylindazol-6-ol (Compound 743, 110 mg). Absolute stereochemistry of the compound was arbitrarily assigned. LCMS: (ES, m/z): 407 [M+H]⁺. ¹H NMR: (300 MHz, DMSO-d₆) δ 9.72 (s, 1H), 9.52 (s, 1H), 8.17 (d, J=9.0 Hz, 1H), 7.96 (d, J=2.9 Hz, 2H), 7.36 (d, J=2.2 Hz, 1H), 6.96 (dd, J=9.0, 7.4 Hz, 1H), 4.63 (s, 3H), 3.82 (d, J=11.2 Hz, 2H), 2.89 (d, J=8.2 Hz, 2H), 2.32 (t, J=11.0 Hz, 3H), 1.08 (d, J=6.2 Hz, 6H).

### Example 267: Synthesis of Compound 748

### Synthesis of Intermediate D204

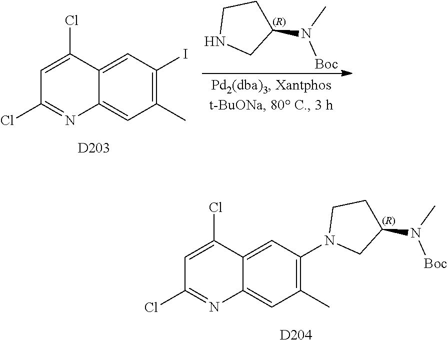

To a solution of 2,4-dichloro-6-iodo-7-methylquinoline (D203, 300 mg, 0.888 mmol, 1 equiv) and tert-butyl N-methyl-N-[(3R)-pyrrolidin-3-yl]carbamate (240 mg, 1.198 mmol, 1.35 equiv) in toluene (8 mL) were added t-BuONa (255.9 mg, 2.664 mmol, 3.0 equiv), XantPhos (102.7 mg, 0.178 mmol, 0.2 equiv) and Pd₂(dba)₃ (81.2 mg, 0.089 mmol, 0.1 equiv). After stirring for 2 h at 80° C. under a nitrogen atmosphere, the resulting mixture was concentrated under reduced pressure. The residue was purified by silica gel column chromatography and eluted with PE/EA (4:1) to afford tert-butyl N-[(3R)-1-(2,4-dichloro-7-methylquinolin-6-yl)pyrrolidin-3-yl]-N-methylcarbamate (D204, 300 mg, 82.36%). LCMS: (ES, m/z): 410 [M+H]

### Synthesis of Intermediate 205

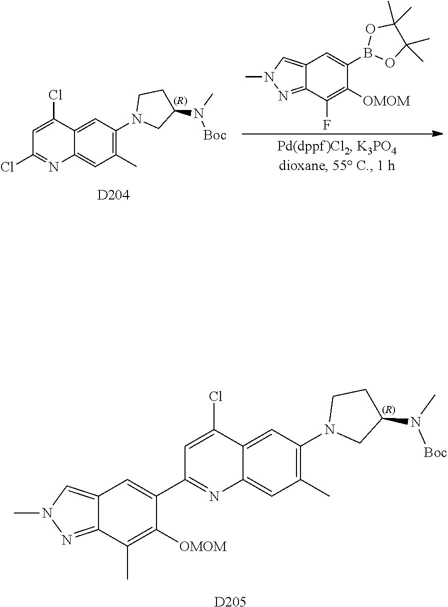

To a solution of Intermediate D204 (150 mg, 0.366 mmol, 1 equiv) and 6-(methoxymethoxy)-2,7-dimethyl-5-(4,4,5,5-tetramethyl-1,3,2-dioxaborolan-2-yl) indazole (121 mg, 0.366 mmol, 1.0 equiv) in dioxane (3 mL) and H₂O (0.8 mL) were added K₃PO₄ (232 mg, 1.098 mmol, 3.0 equiv) and Pd(dppf)Cl₂ (26.7 mg, 0.037 mmol, 0.1 equiv). After stirring for 1 h at 55° C. under a nitrogen atmosphere, the resulting mixture was concentrated under reduced pressure. The residue was purified by silica gel column chromatography and eluted with PE/EA (3:1) to afford tert-butyl N-[(3R)-1-{4-chloro-2-[6-(methoxymethoxy)-2,7-dimethylindazol-5-yl]-7-methylquinolin-6-yl}pyrrolidin-3-yl]-N-methylcarbamate (D205, 124 mg). LCMS: (ES, m/z): 580 [M+H]⁺.

### Synthesis of Compound 748

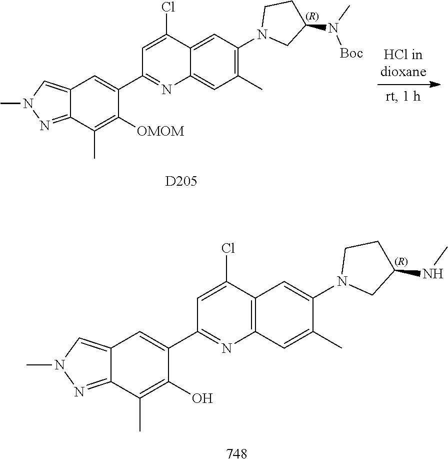

To a stirred solution of tert-butyl Intermediate D205 (100 mg, 0.172 mmol, 1 equiv) in DCM (1 mL) was added 4M HCl (gas) in 1,4-dioxane (1 mL) dropwise at room temperature. The resulting mixture was stirred for 1 h at room temperature and subsequently concentrated under reduced pressure. The residue was purified by reversed-phase flash chromatography following Condition 10, Gradient 8 as previously described to afford 5-{4-chloro-7-methyl-6-[(3R)-3-(methylamino)pyrrolidin-1-yl]quinolin-2-yl}-2,7-dimethylindazol-6-ol (Compound 748, 36 mg). Absolute stereochemistry of the compound was arbitrarily assigned. LCMS: (ES, m/z): 436 [M+H]⁺. ¹H NMR: (300 MHz, DMSO-d₆) δ 14.36 (s, 1H), 8.47 (d, J=13.3 Hz, 2H), 8.34 (s, 1H), 7.82 (d, J=1.0 Hz, 1H), 7.11 (s, 1H), 4.14 (s, 3H), 3.63-3.34 (m, 3H), 3.31-3.14 (m, 2H), 2.59-2.53 (m, 3H), 2.38 (s, 3H), 2.32 (s, 3H), 2.10 (dq, J=12.6, 6.4 Hz, 1H), 1.94 (s, 1H), 1.80 (dt, J=11.9, 6.1 Hz, 1H).

### Example 268: Synthesis of Compound 749

### Synthesis of Intermediate D206

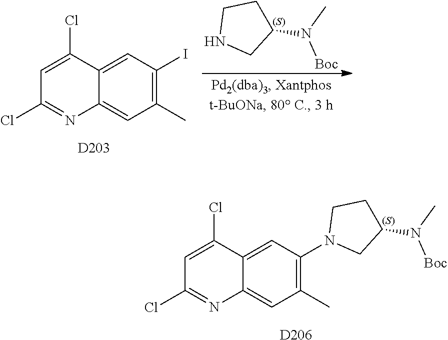

To a solution of Intermediate D203 (300 mg, 0.888 mmol, 1 equiv) and tert-butyl N-methyl-N-[(3S)-pyrrolidin-3-yl]carbamate (231 mg, 1.154 mmol, 1.3 equiv) in toluene (8 mL) were added t-BuONa (256 mg, 2.664 mmol, 3.0 equiv), XantPhos (104 mg, 0.177 mmol, 0.2 equiv) and Pd₂(dba)₃ (81.2 mg, 0.089 mmol, 0.1 equiv). After stirring for 3 h at 80° C. under a nitrogen atmosphere, the resulting mixture was concentrated under reduced pressure. The residue was purified by silica gel column chromatography and eluted with PE/EA (4:1) to afford tert-butyl N-[(3S)-1-(2,4-dichloro-7-methylquinolin-6-yl)pyrrolidin-3-yl]-N-methylcarbamate (D206, 300 mg). LCMS: (ES, m/z): 410 [M+H]⁺.

### Synthesis of Intermediate D207

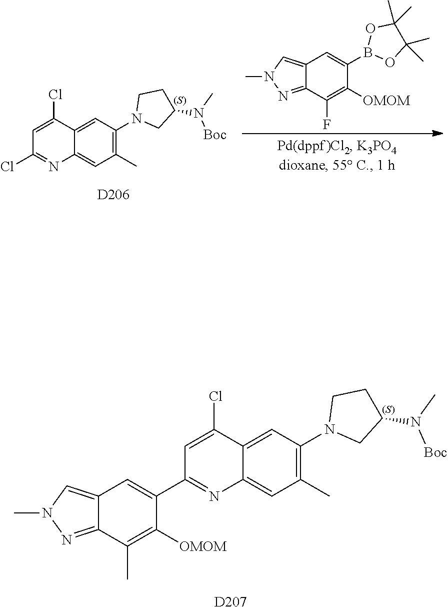

To a solution of Intermediate D206 (150 mg, 0.366 mmol, 1 equiv) and 6-(methoxymethoxy)-2,7-dimethyl-5-(4,4,5,5-tetramethyl-1,3,2-dioxaborolan-2-yl) indazole (121.4 mg, 0.366 mmol, 1.0 equiv) in dioxane (3 mL) and H₂O (0.8 mL) were added K₃PO₄ (232 mg, 1.098 mmol, 3.0 equiv) and Pd(dppf)Cl₂·CH₂Cl₂ (30 mg, 0.037 mmol, 0.1 equiv). After stirring for 1 h at 55° C. under a nitrogen atmosphere, the resulting mixture was concentrated under reduced pressure. The residue was purified by silica gel column chromatography and eluted with PE/EA (3:1) to afford tert-butyl N-[(3S)-1-{4-chloro-2-[6-(methoxymethoxy)-2,7-dimethylindazol-5-yl]-7-methylquinolin-6-yl}pyrrolidin-3-yl]-N-methylcarbamate (D207, 120 mg). LCMS: (ES, m/z): 580 [M+H]⁺.

### Synthesis of Compound 749

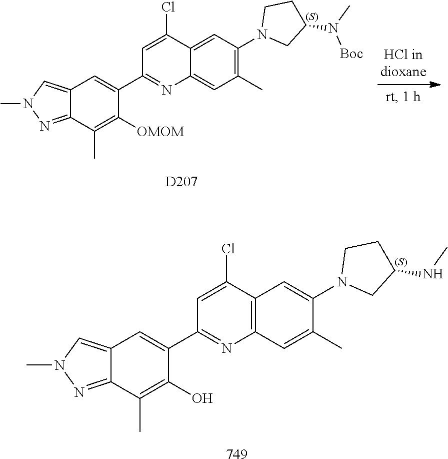

A solution of tert-butyl N-[(3S)-1-{4-chloro-2-[6-(methoxymethoxy)-2,7-dimethylindazol-5-yl]-7-methylquinolin-6-yl}pyrrolidin-3-yl]-N-methylcarbamate (D207, 100 mg, 0.172 mmol, 1 equiv) in DCM (1 mL) was treated with 4M HCl (gas) in 1,4-dioxane (1 mL) for 1 h at room temperature and then concentrated under reduced pressure. The residue was purified by reversed-phase flash chromatography following Condition 10, Gradient 8 as previously described to afford 5-{4-chloro-7-methyl-6-[(3S)-3-(methylamino)pyrrolidin-1-yl]quinolin-2-yl}-2,7-dimethylindazol-6-ol (Compound 749, 36 mg). Absolute stereochemistry of the compound was arbitrarily assigned. LCMS: (ES, m/z): 436 [M+H]⁺. ¹H NMR: (300 MHz, DMSO-d₆) δ 14.36 (s, 1H), 8.47 (d, J=13.3 Hz, 2H), 8.34 (s, 1H), 7.82 (d, J=1.0 Hz, 1H), 7.11 (s, 1H), 4.14 (s, 3H), 3.63-3.34 (m, 3H), 3.31-3.14 (m, 2H), 2.59-2.53 (m, 3H), 2.38 (s, 3H), 2.32 (s, 3H), 2.10 (dq, J=12.6, 6.4 Hz, 1H), 1.94 (s, 1H), 1.80 (dt, J=11.9, 6.1 Hz, 1H).

### Example 269: Synthesis of Compound 677

### Synthesis of Intermediate D208

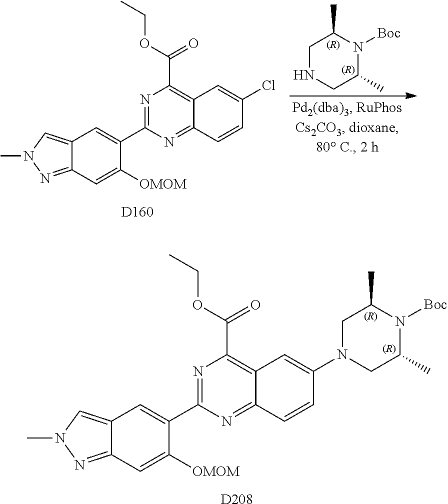

To a stirred mixture of ethyl 6-chloro-2-[6-(methoxymethoxy)-2-methylindazol-5-yl]quinazoline-4-carboxylate (D160, 180 mg, 0.422 mmol, 1 equiv) and tert-butyl (2R,6R)-2,6-dimethylpiperazine-1-carboxylate (108 mg, 0.504 mmol, 1.20 equiv) in dioxane (3 mL) were added RuPhos (39 mg, 0.084 mmol, 0.20 equiv), Pd₂(dba)₃ (39 mg, 0.043 mmol, 0.10 equiv) and Cs₂CO₃ (275 mg, 0.844 mmol, 2.00 equiv) in portions. The resulting mixture was stirred for 2 h at 80° C. under nitrogen atmosphere and then concentrated under reduced pressure. The residue was purified by silica gel column chromatography and eluted with PE/EA (1:1) to afford ethyl 6-[(3R,5R)-4-(tert-butoxycarbonyl)-3,5-dimethylpiperazin-1-yl]-2-[6-(methoxymethoxy)-2-methylindazol-5-yl]quinazoline-4-carboxylate (D208, 150 mg). LCMS: (ES, m/z): 605 [M+H]⁺.

### Synthesis of Intermediate D209

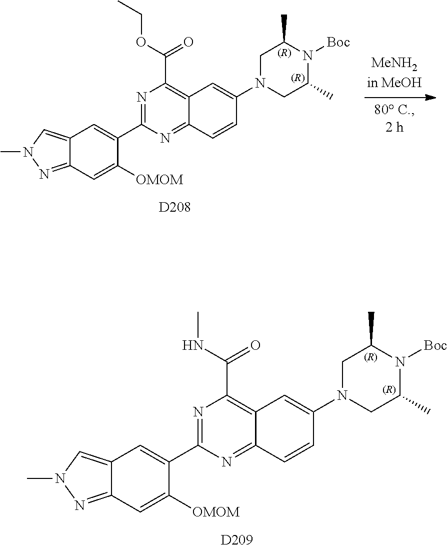

A mixture of Intermediate D208 (140 mg, 0.232 mmol, 1 equiv) and 2M MeNH₂ in MeOH (5 mL) was stirred for 2 h at 80° C. and subsequently concentrated under reduced pressure to afford tert-butyl (2R,6R)-4-{2-[6-(methoxymethoxy)-2-methylindazol-5-yl]-4-(methylcarbamoyl)quinazolin-6-yl}-2,6-dimethylpiperazine-1-carboxylate (D209, 90 mg, crude product) as an orange solid which was used in the next step directly without further purification. LCMS: (ES, m/z): 590 [M+H]⁺.

### Synthesis of Compound 677

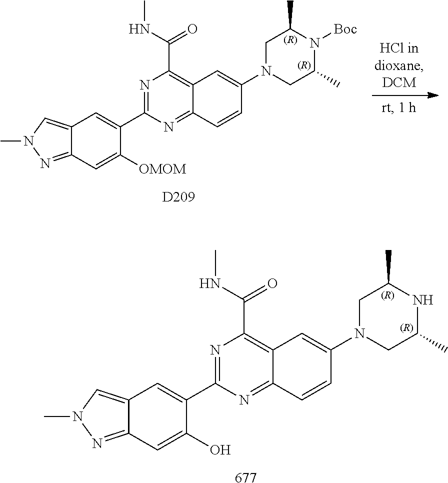

A solution of Intermediate D209 (80 mg, 0.136 mmol, 1 equiv) in DCM (0.8 mL) was treated with 4 M HCl (gas) in 1,4-dioxane (0.4 mL) for 1 h at room temperature and then concentrated under reduced pressure. The residue was purified by reversed-phase flash chromatography following Condition 9, Gradient 5 as previously described to afford 6-[(3R,5R)-3,5-dimethylpiperazin-1-yl]-2-(6-hydroxy-2-methylindazol-5-yl)-N-methylquinazoline-4-carboxamide (Compound 677, 47.3 mg). Absolute stereochemistry of the compound was arbitrarily assigned. LCMS: (ES, m/z): 446 [M+H]⁺. RT=2.303 min on chiral-SFC. ¹H NMR: (400 MHz, DMSO-d₆) δ 13.23 (s, 1H), 9.21 (d, J=8.0 Hz, 2H), 8.44 (s, 1H), 8.13 (s, 1H), 7.96 (d, J=2.3 Hz, 2H), 6.91 (s, 1H), 4.14 (s, 3H), 3.44-3.35 (m, 2H), 3.24 (td, J=6.5, 3.5 Hz, 2H), 2.99 (dd, J=16.4, 5.5 Hz, 5H), 2.17 (s, 1H), 1.13 (d, J=6.4 Hz, 6H).

### Example 270: Synthesis of Compound 676

### Synthesis of Intermediate D210

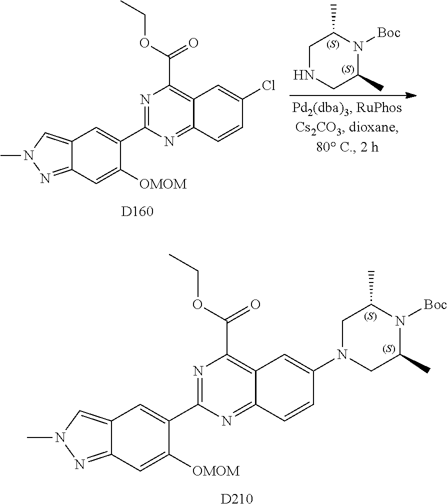

To a stirred solution of ethyl 6-chloro-2-[6-(methoxymethoxy)-2-methylindazol-5-yl]quinazoline-4-carboxylate (D160, 150 mg, 0.351 mmol, 1 equiv) and tert-butyl (2S,6S)-2,6-dimethylpiperazine-1-carboxylate (75 mg, 0.351 mmol, 1 equiv) in 1,4-dioxane (5 ml) were added Pd₂(dba)₃ (32 mg, 0.035 mmol, 0.1 equiv), RuPhos (32 mg, 0.070 mmol, 0.2 equiv) and Cs₂CO₃ (228.99 mg, 0.702 mmol, 2 equiv) at room temperature under nitrogen atmosphere. The resulting mixture was stirred for 2 h at 80° C., quenched with water (5 mL) at room temperature, and extracted with EtOAc (3×10 mL). The combined organic layers were washed with brine (3×10 mL), dried over anhydrous Na₂SO₄, and the filtrate was concentrated under reduced pressure. The residue was purified by silica gel column chromatography and eluted with PE/EA (5:4) to afford ethyl 6-[(3S,5S)-4-(tert-butoxycarbonyl)-3,5-dimethylpiperazin-1-yl]-2-[6-(methoxymethoxy)-2-methylindazol-5-yl]quinazoline-4-carboxylate (D210, 120 mg). LCMS: (ES, m/z): 605 [M+H]⁺.

### Synthesis of Intermediate D211

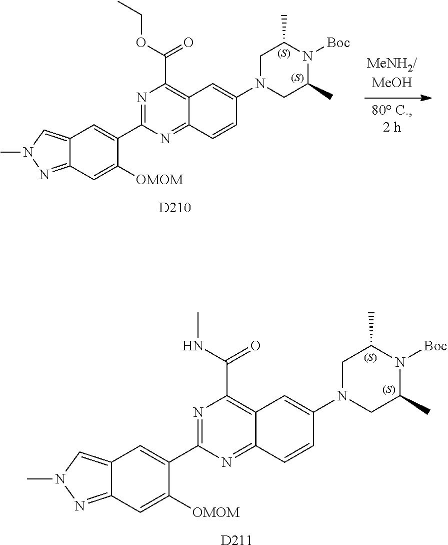

Intermediate D210 (120 mg, 0.198 mmol, 1 equiv) was added to 2M MeNH₂ in MeOH (3 mL) at room temperature, stirred for 2 h at 80° C., and subsequently concentrated under reduced pressure to afford tert-butyl (2S,6S)-4-{2-[6-(methoxymethoxy)-2-methylindazol-5-yl]-4-(methylcarbamoyl)quinazolin-6-yl}-2,6-dimethylpiperazine-1-carboxylate (D211, 85 mg). LCMS: (ES, m/z): 590 [M+H]⁺.

### Synthesis of Compound 676

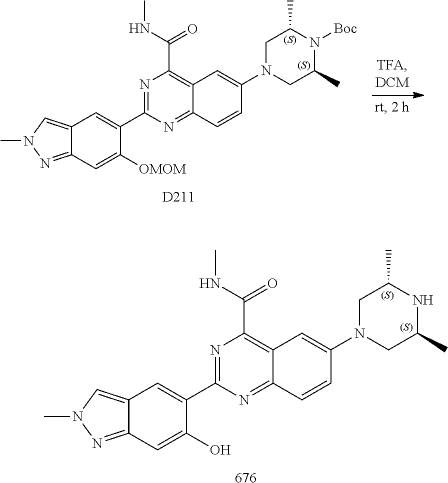

To a mixture of Intermediate D211 (80 mg, 0.136 mmol, 1 equiv) in DCM (3 mL) was added TFA (1 mL) at room temperature. The resulting mixture was stirred 2 h at room temperature and then concentrated under reduced pressure. The residue was purified by reversed-phase flash chromatography following Condition 10, Gradient 10 as previously described to afford 6-[(3S,5S)-3,5-dimethylpiperazin-1-yl]-2-(6-hydroxy-2-methylindazol-5-yl)-N-methylquinazoline-4-carboxamide (Compound 676, 48.8 mg). Absolute stereochemistry of the compound was arbitrarily assigned. LCMS: (ES, m/z): 446 [M+H]⁺. ¹H NMR: (400 MHz, DMSO-d₆) δ 13.22 (s, 1H), 9.23 (s, 2H), 8.44 (s, 1H), 8.13 (s, 1H), 7.96 (d, J=2.4 Hz, 2H), 6.91 (s, 1H), 4.15 (s, 3H), 3.40-3.32 (m, 5H), 2.99 (dd, J=17.0, 5.5 Hz, 5H), 1.13 (d, J=6.4 Hz, 6H).

### Example 271: Synthesis of Compound 652

### Synthesis of Intermediate D212

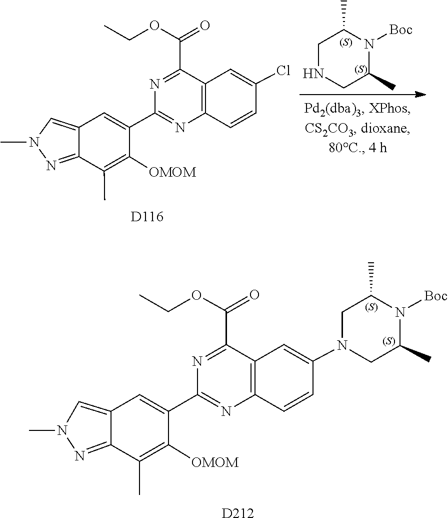

To a stirred solution of ethyl 6-chloro-2-[6-(methoxymethoxy)-2,7-dimethylindazol-5-yl]quinazoline-4-carboxylate (D116, 150 mg, 0.340 mmol, 1 equiv) and tert-butyl (2S,6S)-2,6-dimethylpiperazine-1-carboxylate (73 mg, 0.340 mmol, 1 equiv) in 1,4-dioxane (5 mL) were added Pd₂(dba)₃(31 mg, 0.034 mmol, 0.1 equiv), XPhos (32 mg, 0.068 mmol, 0.2 equiv) and Cs₂CO₃(221.71 mg, 0.680 mmol, 2 equiv) at room temperature under nitrogen atmosphere. The resulting mixture was stirred for 4 h at 80° C. and then concentrated under reduced pressure. The residue was purified by silica gel column chromatography and eluted with PE/EA (5:4) to afford ethyl 6-[(3S,5S)-4-(tert-butoxycarbonyl)-3,5-dimethylpiperazin-1-yl]-2-[6-(methoxymethoxy)-2,7-dimethylindazol-5-yl]quinazoline-4-carboxylate (D212, 160 mg). LCMS: (ES, m/z): 619 [M+H]⁺.

### Synthesis of Intermediate D213

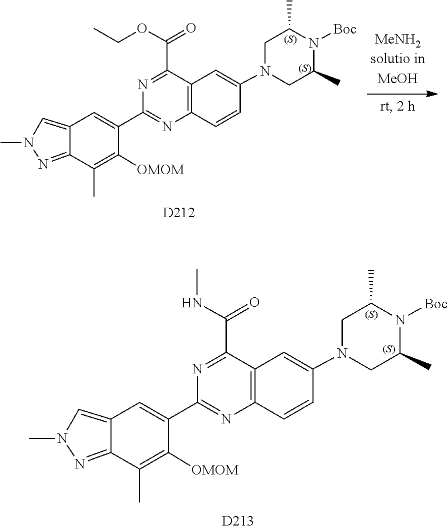

A mixture of Intermediate D212 (150 mg, 0.242 mmol, 1 equiv) and 2 M MeNH₂ solution in MeOH (3 mL) was stirred for 2 h at room temperature and subsequently concentrated under reduced pressure to afford tert-butyl (2S,6S)-4-{2-[6-(methoxymethoxy)-2,7-dimethylindazol-5-yl]-4-(methylcarbamoyl)quinazolin-6-yl}-2,6-dimethylpiperazine-1-carboxylate (D213, 120 mg). LCMS: (ES, m/z): 604 [M+H]⁺.

### Synthesis of Compound 652

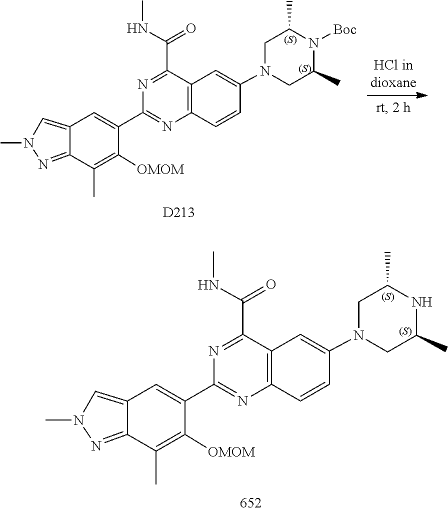

A mixture of Intermediate D213 (85 mg, 0.141 mmol, 1 equiv) and 4M HCl (gas) in 1,4-dioxane (3 ml) was stirred for 2 h at room temperature and then concentrated under reduced pressure. The residue was purified by reversed-phase flash chromatography following Condition 13, Gradient 1 as previously described to afford 6-[(3S,5S)-3,5-dimethylpiperazin-1-yl]-2-(6-hydroxy-2,7-dimethylindazol-5-yl)-N-methylquinazoline-4-carboxamide (Compound 652, 36 mg). Absolute stereochemistry of the compound was arbitrarily assigned. LCMS: (ES, m/z): 460 [M+H]⁺. ¹H NMR: (400 MHz, DMSO-d₆) δ 13.40 (s, 1H), 9.17 (brs, 1H), 9.08 (s, 1H), 8.41 (s, 1H), 8.11 (s, 1H), 7.99-7.94 (m, 2H), 4.16 (s, 3H), 3.41-3.20 (m, 2H), 3.30-3.21 (m, 2H), 3.05-2.94 (m, 5H), 2.41 (s, 3H), 1.14 (d, J=6.5 Hz, 6H).

### Example 272: Synthesis of Compound 669

### Synthesis of Intermediate D214

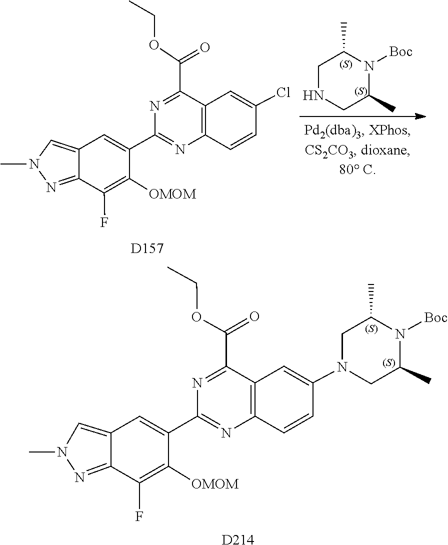

To a stirred solution of ethyl 6-chloro-2-[7-fluoro-6-(methoxymethoxy)-2-methylindazol-5-yl]quinazoline-4-carboxylate (D157, 120 mg, 0.270 mmol, 1 equiv) and tert-butyl (2S,6S)-2,6-dimethylpiperazine-1-carboxylate (58 mg, 0.270 mmol, 1 equiv) in 1,4-dioxane (5 mL) were added Pd₂(dba)₃ (25 mg, 0.027 mmol, 0.1 equiv), XPhos (26 mg, 0.054 mmol, 0.2 equiv) and Cs₂CO₃ (175.78 mg, 0.540 mmol, 2 equiv) at room temperature under nitrogen atmosphere. The resulting mixture was stirred for 4 h at 80° C. and later concentrated reduced pressure. The residue was purified by silica gel column chromatography and eluted with PE/EA (1:5) to afford ethyl 6-[(3 S,5S)-4-(tert-butoxycarbonyl)-3,5-dimethylpiperazin-1-yl]-2-[7-fluoro-6-(methoxymethoxy)-2-methylindazol-5-yl]quinazoline-4-carboxylate (D214, 90 mg). LCMS: (ES, m/z): 623 [M+H]⁺.

### Synthesis of Intermediate D215

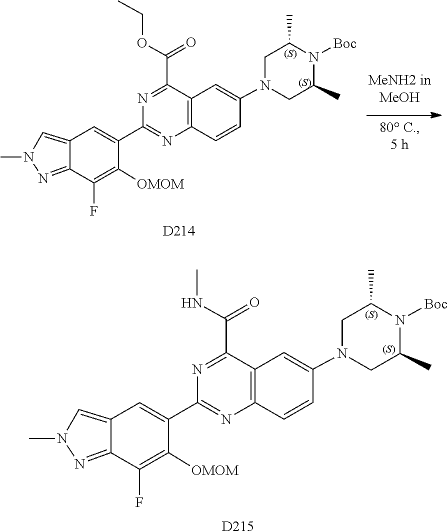

A solution of Intermediate D214 (90 mg, 0.145 mmol, 1 equiv) and 2M MeNH₂ in MeOH (3 mL) was stirred for 5 h at 80° C. and subsequently concentrated under reduced pressure to afford tert-butyl (2S,6S)-4-{2-[7-fluoro-6-(methoxymethoxy)-2-methylindazol-5-yl]-4-(methylcarbamoyl)quinazolin-6-yl}-2,6-dimethylpiperazine-1-carboxylate (D215, 55 mg). LCMS: (ES, m/z): 608 [M+H]⁺.

### Synthesis of Compound 669

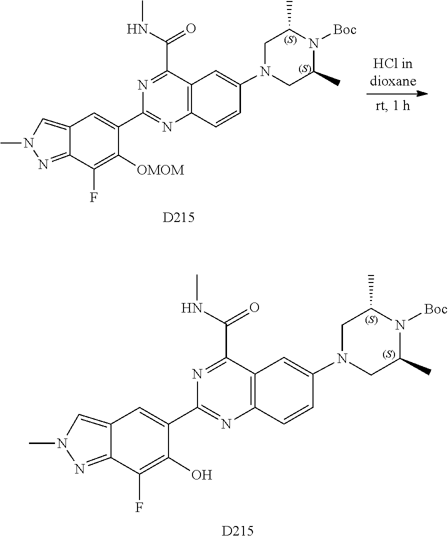

A solution of Intermediate D215 (60 mg, 0.099 mmol, 1 equiv) and 4 M HCl (gas) in 1,4-dioxane (4 mL) was stirred for 1 h at room temperature and then concentrated under reduced pressure. The residue was purified by reversed-phase flash chromatography following Condition 13, Gradient 1 as previously described to afford 6-[(3S,5S)-3,5-dimethylpiperazin-1-yl]-2-(7-fluoro-6-hydroxy-2-methylindazol-5-yl)-N-methylquinazoline-4-carboxamide (Compound 669, 45 mg). Absolute stereochemistry of the compound was arbitrarily assigned. LCMS: (ES, m/z): 464 [M+H]⁺. RT=1.665 min on chiral-SFC. ¹H NMR: (400 MHz, DMSO-d₆) δ 13.43 (s, 1H), 9.21 (d, J=5.1 Hz, 1H), 9.04 (s, 1H), 8.56 (d, J=2.6 Hz, 1H), 8.14 (d, J=2.5 Hz, 1H), 8.05-7.94 (m, 2H), 4.19 (s, 3H), 3.02 (dd, J=11.7, 6.3 Hz, 6H), 2.97 (d, J=4.8 Hz, 3H), 1.13 (d, J=6.5 Hz, 6H).

### Example 273: Synthesis of Compound 744

### Synthesis of Intermediate D217

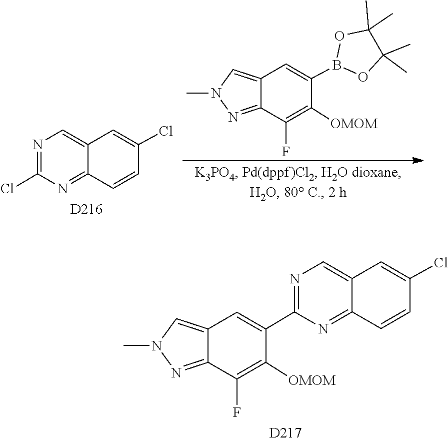

A solution of 2,6-dichloroquinazoline (D216, 420 mg, 2.110 mmol, 1 equiv) in 1,4-dioxane (5 mL)/H₂O (0.5 mL) was treated with 7-fluoro-6-(methoxymethoxy)-2-methyl-5-(4,4,5,5-tetramethyl-1,3,2-dioxaborolan-2-yl) indazole (851.28 mg, 2.532 mmol, 1.2 equiv), K₂CO₃ (874.93 mg, 6.330 mmol, 3 equiv) and Pd(dppf)Cl₂ (154.41 mg, 0.211 mmol, 0.1 equiv) for 2 h at 80° C. under nitrogen atmosphere. Water (30 mL) was then added before the mixture was extracted with DCM (2×30 mL). The combined organic layers were dried over anhydrous Na₂SO₄, and the filtrate was concentrated under reduced pressure. The residue was purified by silica gel column chromatography and eluted with PE/EA (2:3) to afford 6-chloro-2-[7-fluoro-6-(methoxymethoxy)-2-methylindazol-5-yl]quinazoline (D217, 522 mg). LCMS: (ES, m/z): 373 [M+H]⁺.

### Synthesis of Intermediate D218

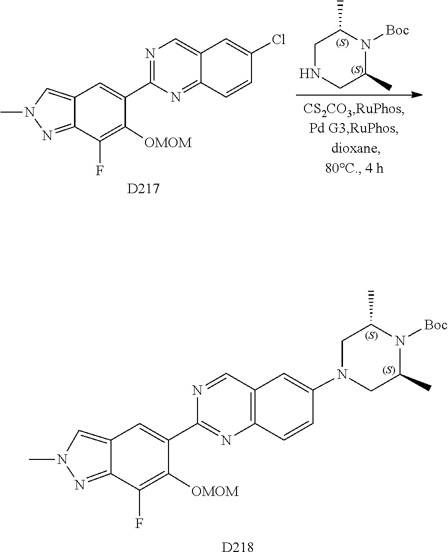

A solution of Intermediate D217 (170 mg, 0.456 mmol, 1 equiv) in 1,4-dioxane (2 mL) was treated with tert-butyl (2S,6S)-2,6-dimethylpiperazine-1-carboxylate (117.28 mg, 0.547 mmol, 1.2 equiv), Cs₂CO₃ (297.17 mg, 0.912 mmol, 2 equiv), RuPhos (42.56 mg, 0.091 mmol, 0.2 equiv) and RuPhos Palladacycle Gen.3 (38.14 mg, 0.046 mmol, 0.1 equiv) for 2 h at 80° C. under nitrogen atmosphere. Water (15 mL) was then added and the mixture was extracted with DCM (2×20 mL). The combined organic layers were dried over anhydrous Na₂SO₄, and the filtrate was concentrated under reduced pressure. The residue was purified by silica gel column chromatography and eluted with PE/EA (1:1) to afford tert-butyl (2S,6S)-4-{2-[7-fluoro-6-(methoxymethoxy)-2-methylindazol-5-yl]quinazolin-6-yl}-2,6-dimethylpiperazine-1-carboxylate (D218, 250 mg). LCMS: (ES, m/z): 551 [M+H]⁺.

### Synthesis of Compound 744

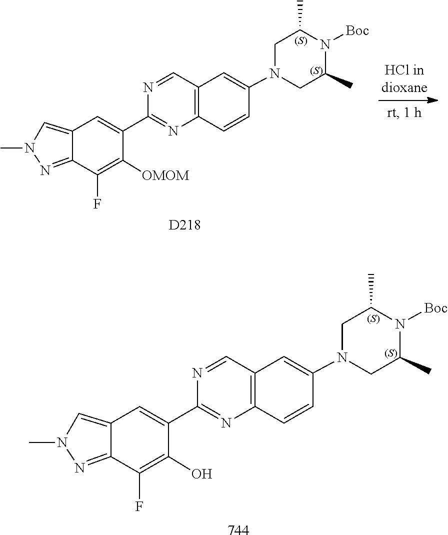

A solution of Intermediate D218 (270 mg, 0.490 mmol, 1 equiv) in DCM (2.7 mL) was treated with HCl (gas) in 1,4-dioxane (1.4 mL, 4 M) for 1 h at room temperature and subsequently concentrated under reduced pressure. The crude product was purified by Prep-HPLC following Condition 6, Gradient 12 as previously described to afford 5-{6-[(3S,5S)-3,5-dimethylpiperazin-1-yl]quinazolin-2-yl}-7-fluoro-2-methylindazol-6-ol (Compound 744, 40 mg). Absolute stereochemistry of the compound was arbitrarily assigned. LCMS: (ES, m/z): 407 [M+H]⁺. ¹H NMR: (400 MHz, DMSO-d₆) δ 13.69 (s, 1H), 9.51 (s, 1H), 8.83 (d, J=1.0 Hz, 1H), 8.55 (d, J=2.6 Hz, 1H), 7.97-7.87 (m, 2H), 7.37 (d, J=2.3 Hz, 1H), 4.17 (s, 3H), 3.38 (dd, J=11.8, 3.4 Hz, 2H), 3.23 (qd, J=6.4, 3.3 Hz, 2H), 3.02 (dd, J=11.8, 6.3 Hz, 2H), 2.13 (s, 1H), 1.13 (d, J=6.5 Hz, 6H).

### Example 274: Synthesis of Compound 745

### Synthesis of Intermediate D219

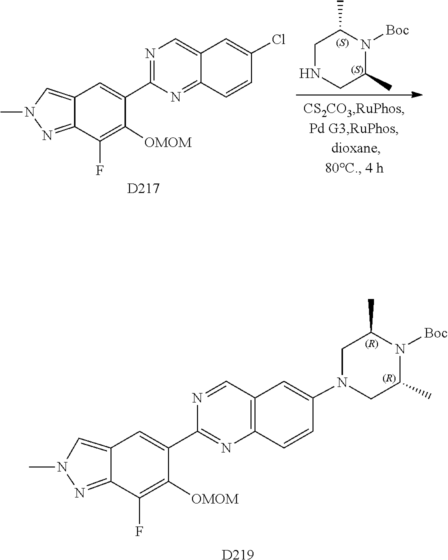

A solution of 6-chloro-2-[7-fluoro-6-(methoxymethoxy)-2-methylindazol-5-yl]quinazoline (D217, 170.0 mg, 0.456 mmol, 1.0 equiv) in 1,4-dioxane (2 mL) was treated with tert-butyl (2R,6R)-2,6-dimethylpiperazine-1-carboxylate (117.3 mg, 0.547 mmol, 1.2 equiv), Cs₂CO₃ (297.2 mg, 0.912 mmol, 2.0 equiv), RuPhos (42.5 mg, 0.091 mmol, 0.2 equiv) and RuPhos Palladacycle Gen.3 (38.14 mg, 0.046 mmol, 0.1 equiv) for 2 h at 80° C. under nitrogen atmosphere. Water (15 mL) was subsequently added, and then the mixture was extracted with DCM (2×20 mL). The combined organic layers were dried over anhydrous Na₂SO₄ and the filtrate was concentrated under reduced pressure. The residue was purified by silica gel column chromatography and eluted with PE/EA (1:1) to afford tert-butyl (2R,6R)-4-{2-[7-fluoro-6-(methoxymethoxy)-2-methylindazol-5-yl]quinazolin-6-yl}-2,6-dimethylpiperazine-1-carboxylate (D219, 220 mg). LCMS: (ES, m/z):551 [M+H]⁺.

### Synthesis of Compound 745

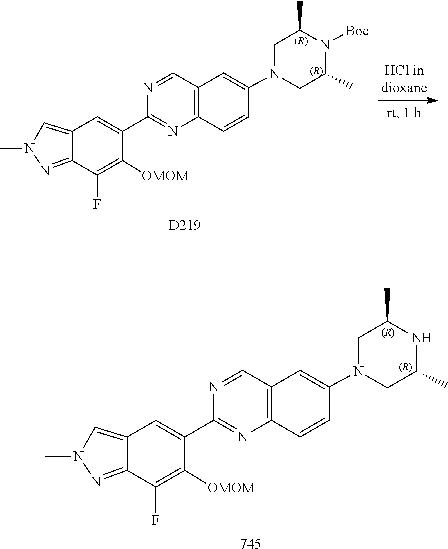

A solution of Intermediate D219 (220 mg, 0.400 mmol, 1 equiv) in DCM (2.2 mL) was treated with HCl(gas) in 1,4-dioxane (1.1 mL, 4M) for 1 h at room temperature and subsequently concentrated under reduced pressure. The crude product was purified by Prep-HPLC following Condition 20, Gradient 1 as previously described to afford 5-{6-[(3R,5R)-3,5-dimethylpiperazin-1-yl]quinazolin-2-yl}-7-fluoro-2-methylindazol-6-ol (Compound 745, 55 mg). Absolute stereochemistry of the compound was arbitrarily assigned. LCMS: (ES, m/z):407 [M+H]⁺. 1H NMR: (400 MHz, DMSO-d₆) δ 13.69 (s, 1H), 9.52 (s, 1H), 8.84 (d, J=1.0 Hz, 1H), 8.55 (d, J=2.6 Hz, 1H), 7.98-7.88 (m, 2H), 7.38 (d, J=2.3 Hz, 1H), 4.17 (s, 3H), 3.38 (dd, J=11.7, 3.4 Hz, 2H), 3.24 (qd, J=6.4, 3.3 Hz, 2H), 3.03 (dd, J=11.8, 6.3 Hz, 2H), 1.13 (d, J=6.4 Hz, 6H).

### Example 275: Synthesis of Compound 660

### Synthesis of Intermediate D220

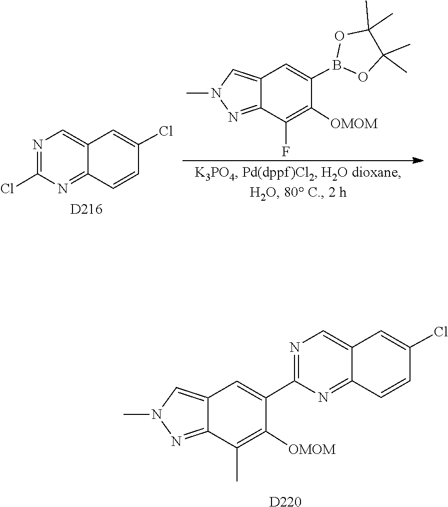

To a solution of 2,6-dichloroquinazoline (D216, 300 mg, 1.507 mmol, 1.0 equiv) and 6-(methoxymethoxy)-2,7-dimethyl-5-(4,4,5,5-tetramethyl-1,3,2-dioxaborolan-2-yl)-2H-indazole (600 mg, 1.809 mmol, 1.2 equiv) in dioxane (10 mL) and H₂O (2 mL) were added K₃PO₄ (640 mg, 3.014 mmol, 2.0 equiv) and Pd(dppf)Cl₂ (110 mg, 0.1507 mmol, 0.1 equiv) at room temperature. After stirring for 2 h at 70° C. under nitrogen atmosphere, the resulting mixture was concentrated under reduced pressure. The residue was purified by silica gel chromatography and eluted with PE/EA (1:1) to afford 6-chloro-2-(6-(methoxymethoxy)-2,7-dimethyl-2H-indazol-5-yl)quinazoline (D220, 450 mg, 81.1%) as a yellow solid. LCMS: (ES, m/z): 369 [M+H]⁺.

### Synthesis of Intermediate D221

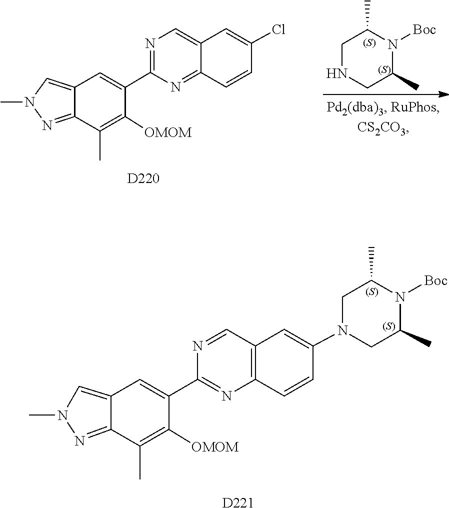

To a stirred solution of Intermediate D220 (120 mg, 0.325 mmol, 1 equiv) and tert-butyl (2S,6S)-2,6-dimethylpiperazine-1-carboxylate (84 mg, 0.390 mmol, 1.2 equiv) in dioxane (5 mL) were added RuPhos (31 mg, 0.065 mmol, 0.2 equiv), Cs₂CO₃ (212 mg, 0.650 mmol, 2 equiv) and Pd₂(dba)₃ (30 mg, 0.033 mmol, 0.1 equiv) at room temperature. The resulting mixture was stirred for 3 h at 80° C. under nitrogen atmosphere and subsequently concentrated under reduced pressure. The residue was purified by silica gel column chromatography and eluted with PE/EA (5:95) to afford tert-butyl (2S,6S)-4-{2-[6-(methoxymethoxy)-2,7-dimethylindazol-5-yl]quinazolin-6-yl}-2,6-dimethylpiperazine-1-carboxylate (D221, 100 mg). LCMS: (ES, m/z): 547 [M+H]⁺.

### Synthesis of Compound 660

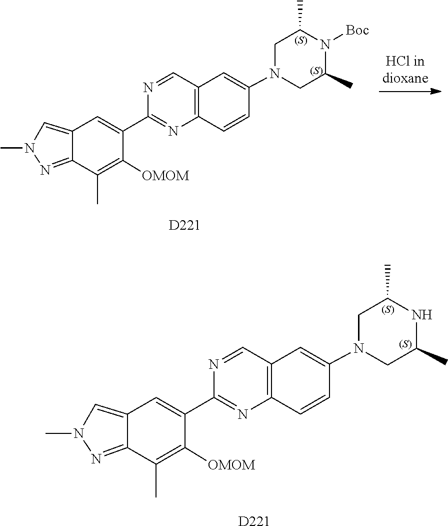

A mixture of Intermediate D221 (80 mg, 0.146 mmol, 1 equiv) and 4M HCl (gas) in 1,4-dioxane (2 mL) was stirred for 1 h at room temperature and then concentrated under reduced pressure. The residue was purified by reversed-phase flash chromatography following Condition 13, Gradient 1 as previously described to afford 5-{6-[(3S,5S)-3,5-dimethylpiperazin-1-yl]quinazolin-2-yl}-2,7-dimethylindazol-6-ol (Compound 660, 30 mg). Absolute stereochemistry of the compound was arbitrarily assigned. LCMS: (ES, m/z): 403 [M+H]⁺. ¹H NMR: (300 MHz, DMSO-d₆) δ 13.64 (s, 1H), 9.50 (s, 1H), 8.90 (s, 1H), 8.40 (s, 1H), 7.91 (s, 2H), 7.37 (s, 1H), 4.14 (s, 3H), 3.38 (s, 2H), 3.24 (s, 2H), 3.01 (dd, J=11.6, 6.2 Hz, 2H), 2.39 (s, 3H), 1.13 (d, J=6.4 Hz, 6H).

### Example 276: Synthesis of Compound 661

### Synthesis of Intermediate D222

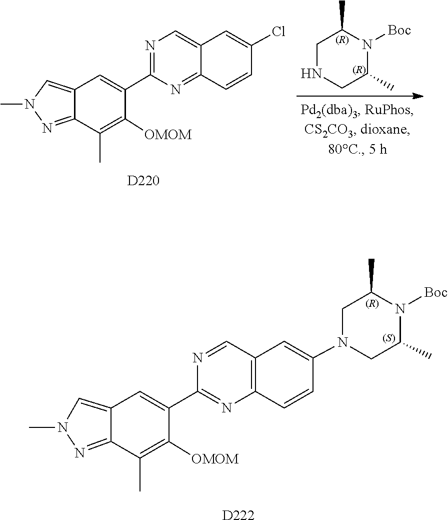

To a stirred mixture of 6-chloro-2-(6-(methoxymethoxy)-2,7-dimethyl-2H-indazol-5-yl)quinazoline (D220, 150 mg, 0.407 mmol, 1 equiv) and tert-butyl (2R,6R)-2,6-dimethylpiperazine-1-carboxylate (105 mg, 0.490 mmol, 1.2 equiv) in dioxane (3 mL) were added RuPhos (38 mg, 0.081 mmol, 0.2 equiv), Pd₂(dba)₃ (37 mg, 0.040 mmol, 0.1 equiv) and Cs₂CO₃ (265 mg, 0.813 mmol, 2.0 equiv) at room temperature under nitrogen atmosphere. The resulting mixture was stirred for 5 h at 80° C. under nitrogen atmosphere and subsequently concentrated under reduced pressure. The residue was purified by silica gel column chromatography and eluted with PE/EA (1:1) to afford tert-butyl (2R,6R)-4-{2-[6-(methoxymethoxy)-2,7-dimethylindazol-5-yl]quinazolin-6-yl}-2,6-dimethylpiperazine-1-carboxylate (D222, 120 mg). LCMS: (ES, m/z): 547 [M+H]⁺.

### Synthesis of Compound 661

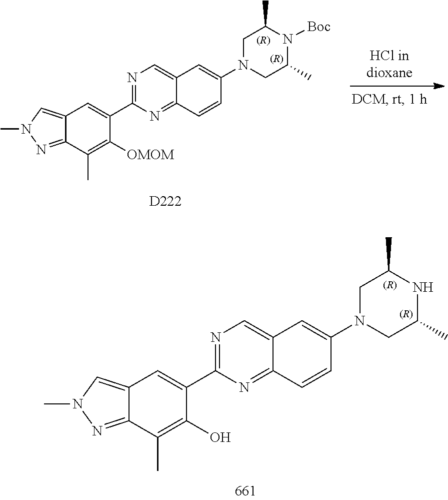

A solution of Intermediate D222 (100 mg, 0.188 mmol, 1 equiv) in DCM (2.0 mL) was treated with 4 M HCl (gas) in 1,4-dioxane (0.8 mL) for 1 h at room temperature and then concentrated under reduced pressure. The residue was purified by reverse-phase flash chromatography following Condition 9, Gradient 5 as previously described to afford 5-(6-((3R,5R)-3,5-dimethylpiperazin-1-yl)quinazolin-2-yl)-2,7-dimethyl-2H-indazol-6-ol (Compound 661, 38.8 mg). Absolute stereochemistry of the compound was arbitrarily assigned. LCMS: (ES, m/z): 403 [M+H]⁺. RT=2.374 min on chiral-SFC. ¹H NMR: (400 MHz, DMSO-d₆) δ 13.64 (s, 1H), 9.51 (s, 1H), 8.90 (s, 1H), 8.40 (s, 1H), 7.91 (d, J=2.2 Hz, 2H), 7.37 (s, 1H), 4.14 (s, 3H), 3.36 (dd, J=11.7, 3.4 Hz, 2H), 3.29-3.19 (m, 2H), 3.01 (dd, J=11.8, 6.2 Hz, 2H), 2.40 (s, 3H), 1.13 (d, J=6.4 Hz, 6H).

### Example 277: Synthesis of Compound 750

### Synthesis of Intermediate D223

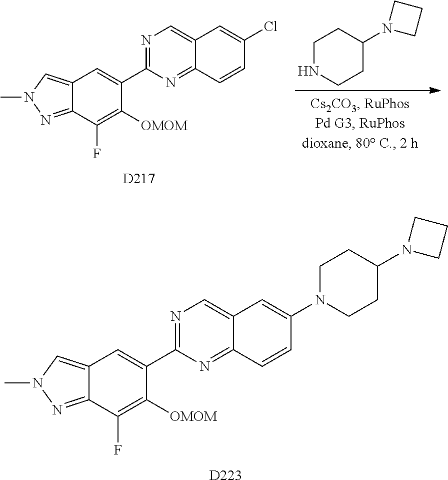

A solution of 6-chloro-2-[7-fluoro-6-(methoxymethoxy)-2-methylindazol-5-yl]quinazoline (D217, 100.0 mg, 0.268 mmol, 1.0 equiv) in 1,4-dioxane (1.5 mL) was treated with 4-(azetidin-1-yl)piperidine dihydrochloride (68.6 mg, 0.322 mmol, 1.2 equiv), Cs₂CO₃ (349.6 mg, 1.072 mmol, 4.0 equiv), RuPhos (25.0 mg, 0.054 mmol, 0.2 equiv) and RuPhos Palladacycle Gen.3 (22.4 mg, 0.027 mmol, 0.1 equiv) for 2 h at 80° C. under nitrogen atmosphere. Water (15 ml) was added to the mixture, which was subsequently extracted with DCM (2×20 mL). The combined organic layers were dried over anhydrous Na₂SO₄, and the filtrate was concentrated under reduced pressure. The residue was purified by silica gel column chromatography and eluted with DCM/MeOH (10:1) to afford 6-[4-(azetidin-1-yl)piperidin-1-yl]-2-[7-fluoro-6-(methoxymethoxy)-2-methylindazol-5-yl]quinazoline (D223, 120 mg). LCMS: (ES, m/z): 477 [M+H]⁺.

### Synthesis of Compound 750

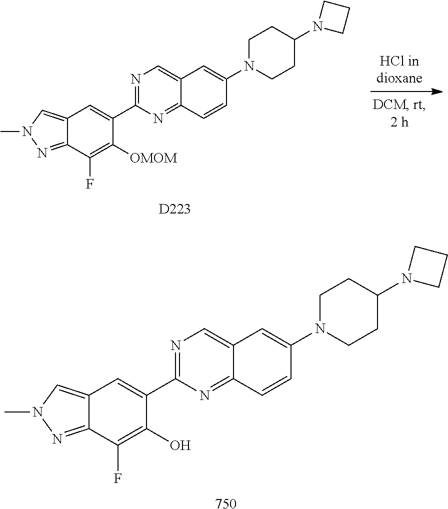

A solution of Intermediate D223 (120 mg, 0.252 mmol, 1 equiv) in DCM (1.2 mL) was treated with HCl (gas) in 1,4-dioxane (0.6 mL, 4M) for 1 h at room temperature, subsequently concentrated under reduced pressure, acidified to pH 8 with saturated NaHCO₃ (aq.), and then extracted with DCM (3×20 mL). The combined organic layers were dried over anhydrous Na₂SO₄, and the filtrate was concentrated under reduced pressure. The residue was purified by trituration with MeCN (10 mL). The resulting solid was dried by air to afford 5-{6-[4-(azetidin-1-yl)piperidin-1-yl]quinazolin-2-yl}-7-fluoro-2-methylindazol-6-ol (Compound 750, 65 mg). LCMS: (ES, m/z): 433 [M+H]⁺. ¹H NMR: (400 MHz, DMSO-d₆) δ 13.70 (s, 1H), 9.52 (s, 1H), 8.84 (d, J=1.1 Hz, 1H), 8.55 (d, J=2.7 Hz, 1H), 7.93 (d, J=1.6 Hz, 2H), 7.40 (d, J=1.7 Hz, 1H), 4.17 (s, 3H), 3.76 (dd, J=11.1, 6.5 Hz, 2H), 3.11 (t, J=6.8 Hz, 4H), 3.03 (ddd, J=12.9, 9.9, 3.0 Hz, 2H), 2.21 (dt, J=9.0, 4.9 Hz, 1H), 1.94 (p, J=6.8 Hz, 2H), 1.75 (d, J=12.8 Hz, 2H), 1.35-1.28 (m, 2H), 1.24 (d, J=6.0 Hz, 1H), 1.18 (s, 2H), 0.83-0.71 (m, 2H).

### Example 278: Synthesis of Compound 751

### Synthesis of Intermediate D224

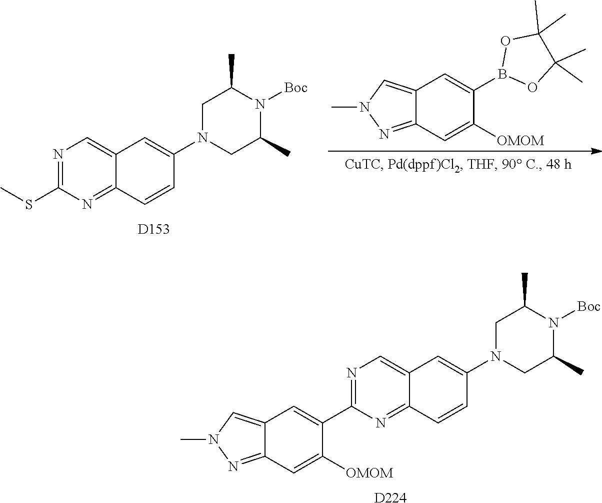

To a stirred solution of tert-butyl (2R,6S)-2,6-dimethyl-4-[2-(methylsulfanyl)quinazolin-6-yl]piperazine-1-carboxylate (D153, 250 mg, 0.643 mmol, 1 equiv) and 6-(methoxymethoxy)-2-methyl-5-(4,4,5,5-tetramethyl-1,3,2-dioxaborolan-2-yl) indazole (410 mg, 1.286 mmol, 2 equiv) in THF (10 mL) were added CuTC (304 mg, 1.415 mmol, 2.2 equiv) and Pd(dppf)Cl₂ (47 mg, 0.064 mmol, 0.1 equiv) at room temperature under nitrogen atmosphere. The resulting mixture was stirred for 48 h at 90° C. under nitrogen atmosphere, allowed to cool to room temperature, quenched by the addition of water (50 mL) at room temperature, and then extracted with CH₂Cl₂ (3×20 mL). The combined organic layers were washed with brine (1×30 mL), dried over anhydrous Na₂SO₄, and the filtrate was concentrated under reduced pressure. The residue was purified by reversed-phase flash chromatography following Condition 10, Gradient 22 as previously described to afford tert-butyl (2R,6S)-4-{2-[6-(methoxymethoxy)-2-methylindazol-5-yl]quinazolin-6-yl}-2,6-dimethylpiperazine-1-carboxylate (D224, 66 mg). LCMS: (ES, m/z): 533 [M+H]⁺.

### Synthesis of Compound 751

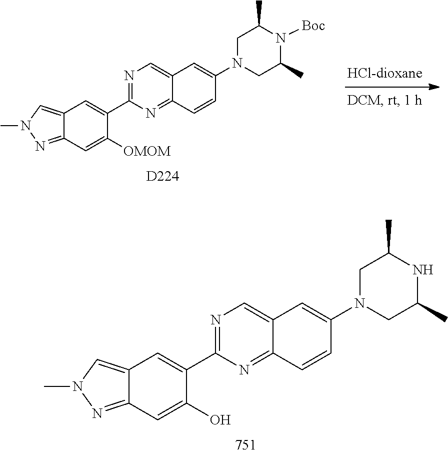

A solution of Intermediate D224 (66 mg, 0.124 mmol, 1 equiv) and HCl (gas) in 1,4-dioxane (0.35 mL) in DCM (1 mL) was stirred for 1 h at room temperature under nitrogen atmosphere and subsequently concentrated under vacuum. The residue was purified by reversed-phase flash chromatography following Condition 14, Gradient 1 as previously described to afford 5-{6-[(3R,5S)-3,5-dimethylpiperazin-1-yl]quinazolin-2-yl}-2-methylindazol-6-ol hydrochloride (Compound 751, 8 mg). Absolute stereochemistry of the compound was arbitrarily assigned. LCMS: (ES, m/z): 389 [M−HCl+H]⁺. ¹H NMR: (300 MHz, DMSO-d₆) δ 9.54 (s, 1H), 9.05 (s, 1H), 8.43 (s, 1H), 7.98 (s, 2H), 7.52 (s, 1H), 6.91 (s, 1H), 4.12 (d, J=8.0 Hz, 5H), 3.44 (s, 2H), 2.85 (t, J=12.4 Hz, 2H), 1.33 (d, J=6.5 Hz, 6H).

### Example 279: Synthesis of Compound 756

### Synthesis of Intermediate D225

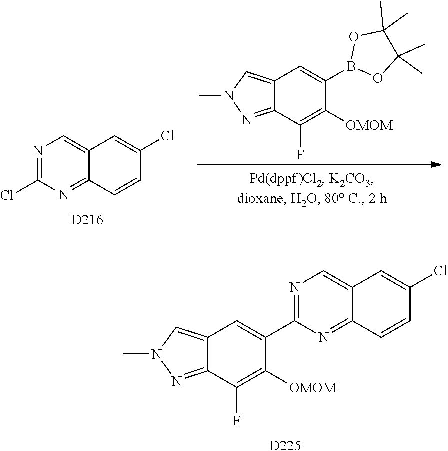

To a stirred solution of 2,6-dichloroquinazoline (D216, 300 mg, 1.507 mmol, 1 equiv), K₂CO₃ (417 mg, 3.014 mmol, 2 equiv) and 7-fluoro-6-(methoxymethoxy)-2-methyl-5-(4,4,5,5-tetramethyl-1,3,2-dioxaborolan-2-yl) indazole (608 mg, 1.808 mmol, 1.2 equiv) in dioxane (5 mL) were added H₂O (0.5 mL) and Pd(dppf)Cl₂ (111 mg, 0.151 mmol, 0.1 equiv) at room temperature under nitrogen atmosphere. The resulting mixture was stirred for 2 h at 80° C. under nitrogen atmosphere and allowed to cool to room temperature. The reaction was quenched by the addition of water (50 mL) at room temperature and subsequently extracted with CH₂Cl₂ (3×10 mL). The combined organic layers were washed with brine (1×10 mL), dried over anhydrous Na₂SO₄, and the filtrate was concentrated under reduced pressure. The residue was purified by silica gel column chromatography and eluted with PE/THE (1:1) to afford 6-chloro-2-[7-fluoro-6-(methoxymethoxy)-2-methylindazol-5-yl]quinazoline (D225, 320 mg). LCMS: (ES, m/z): 373 [M+H]⁺.

### Synthesis of Intermediate D226

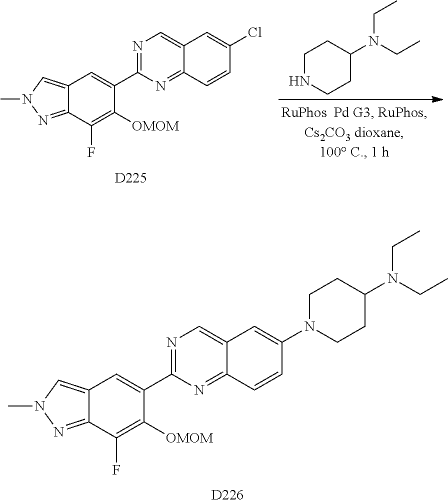

To a stirred solution of Intermediate D225 (160 mg, 0.429 mmol, 1 equiv) and N,N-diethylpiperidin-4-amine (134 mg, 0.858 mmol, 2 equiv) in dioxane (5 mL) were added Cs₂CO₃ (280 mg, 0.858 mmol, 2 equiv), RuPhos (40 mg, 0.086 mmol, 0.2 equiv) and RuPhos Palladacycle Gen.3 (36 mg, 0.043 mmol, 0.1 equiv) at room temperature under nitrogen atmosphere. The resulting mixture was stirred for 1 h at 100° C. under nitrogen atmosphere and allowed to cool to room temperature. The reaction was quenched by the addition of water (50 mL) at room temperature and subsequently extracted with CH₂Cl₂ (3×10 mL). The combined organic layers were washed with brine (1×10 mL), dried over anhydrous Na₂SO₄, and the filtrate was concentrated under reduced pressure. The residue was purified by silica gel column chromatography and eluted with THE to afford N,N-diethyl-1-{2-[7-fluoro-6-(methoxymethoxy)-2-methylindazol-5-yl]quinazolin-6-yl}piperidin-4-amine (D226, 180 mg). LCMS: (ES, m/z): 493 [M+H]⁺.

### Synthesis of Compound 756

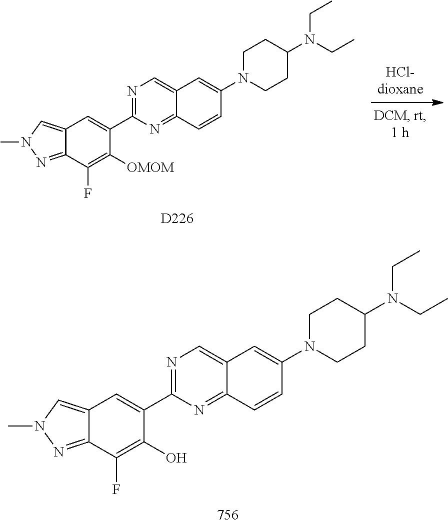

A solution of Intermediate D226 (180 mg, 0.365 mmol, 1 equiv) and 4M HCl (gas) in 1,4-dioxane (1 mL) in DCM (3 mL) was stirred for 1 h at room temperature and then concentrated under reduced pressure. The residue was purified by reversed-phase flash chromatography following Condition 10, Gradient 21 as previously described to afford 5-{6-[4-(diethylamino)piperidin-1-yl]quinazolin-2-yl}-7-fluoro-2-methylindazol-6-ol (Compound 756, 70 mg). LCMS: (ES, m/z): 449 [M+H]⁺. ¹H NMR: (300 MHz, DMSO-d₆) δ 13.70 (s, 1H), 9.53 (s, 1H), 8.84 (d, J=1.0 Hz, 1H), 8.56 (d, J=2.6 Hz, 1H), 7.95 (s, 2H), 7.42 (s, 1H), 4.17 (s, 3H), 4.02 (d, J=12.7 Hz, 2H), 3.33 (s, 2H), 2.90 (t, J=11.9 Hz, 2H), 2.73 (s, 1H), 2.55 (d, J=7.0 Hz, 2H), 1.82 (d, J=12.6 Hz, 2H), 1.57 (t, J=11.6 Hz, 2H), 0.98 (t, J=7.1 Hz, 6H).

### Example 280: Synthesis of Compound 757

### Synthesis of Intermediate D227

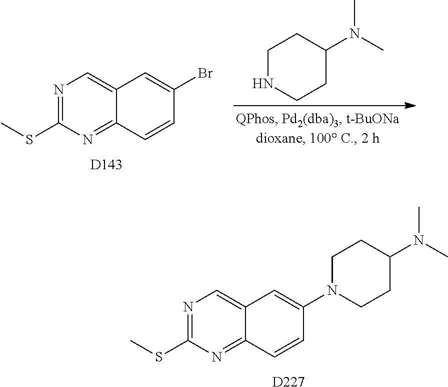

To a stirred solution of 6-bromo-2-(methylsulfanyl)quinazoline (D143, 400 mg, 1.568 mmol, 1 equiv) and N,N-dimethylpiperidin-4-amine (302 mg, 2.352 mmol, 1.5 equiv) in dioxane (10 mL) were added t-BuONa (302 mg, 3.136 mmol, 2 equiv), 1,2,3,4,5-pentaphenyl-1-(di-tert-butylphosphino)ferrocene (223 mg, 0.314 mmol, 0.2 equiv) and Pd₂(dba)₃ (144 mg, 0.157 mmol, 0.1 equiv) at room temperature under nitrogen atmosphere. The resulting mixture was stirred for 3 h at 100° C. under nitrogen atmosphere and subsequently concentrated under reduced pressure. The residue was purified by silica gel column chromatography and eluted with CH₂Cl₂/MeOH (10:1) to afford N,N-dimethyl-1-[2-(methylsulfanyl)quinazolin-6-yl]piperidin-4-amine (D227, 330 mg, 69.59%) as a red solid. LCMS: (ES, m/z): 303 [M+H]⁺.

### Synthesis of Intermediate D228

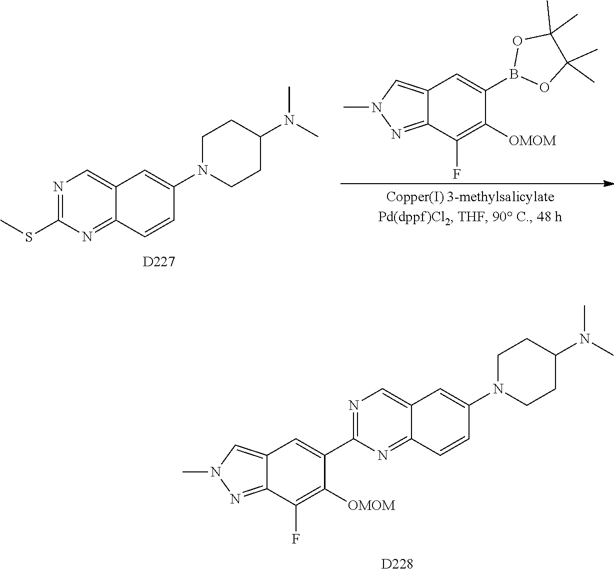

To a stirred solution of Intermediate D227 (330 mg, 1.091 mmol, 1 equiv) and 7-fluoro-6-(methoxymethoxy)-2-methyl-5-(4,4,5,5-tetramethyl-1,3,2-dioxaborolan-2-yl) indazole (367 mg, 1.091 mmol, 1 equiv) in THF (10 mL) were added Copper(I) 3-methylsalicylate (517 mg, 2.400 mmol, 2.2 equiv) and Pd(dppf)Cl₂·CH₂Cl₂ (178 mg, 0.218 mmol, 0.2 equiv) at room temperature under nitrogen atmosphere. The resulting mixture was stirred for 24 h at 90° C. under nitrogen atmosphere and then concentrated under reduced pressure. The residue was purified by silica gel column chromatography and eluted with CH₂Cl₂/MeOH (10:1) to afford 1-{2-[7-fluoro-6-(methoxymethoxy)-2-methylindazol-5-yl]quinazolin-6-yl}-N,N-dimethylpiperidin-4-amine (D228, 100 mg). LCMS: (ES, m/z): 465 [M+H]⁺.

### Synthesis of Compound 757

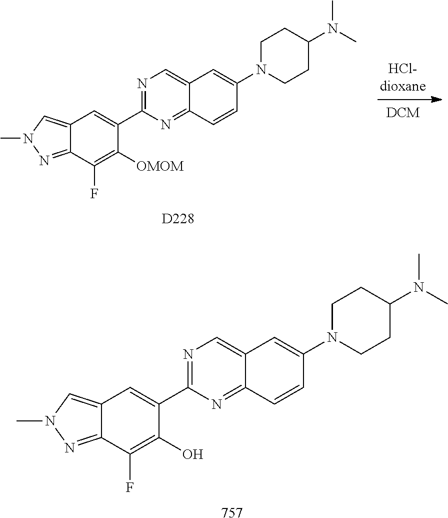

A solution of Intermediate D228 (100 mg, 0.215 mmol, 1 equiv) and HCl (gas) in 1,4-dioxane (1 mL) in DCM (5 mL) was stirred for 2 h at room temperature and subsequently concentrated under reduced pressure. The residue was purified by reversed-phase flash chromatography following Condition 10, Gradient 3 as previously described to afford 5-{6-[4-(dimethylamino)piperidin-1-yl]quinazolin-2-yl}-7-fluoro-2-methylindazol-6-ol (Compound 757, 25 mg). LCMS: (ES, m/z): 421 [M+H]⁺. ¹H NMR: (400 MHz, DMSO-d₆) δ 13.70 (s, 1H), 9.53 (s, 1H), 8.85 (s, 1H), 8.56 (d, J=2.7 Hz, 1H), 7.95 (s, 2H), 7.42 (s, 1H), 4.17 (s, 3H), 3.98 (d, J=12.6 Hz, 2H), 2.91 (t, J=12.0 Hz, 2H), 2.35-2.22 (m, 1H), 2.21 (s, 6H), 1.90 (d, J=12.5 Hz, 2H), 1.52 (q, J=11.9, 11.1 Hz, 2H).

### Example 281: Synthesis of Compound 758

### Synthesis of Intermediate D230

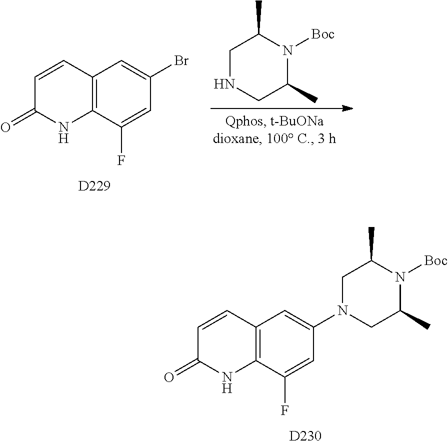

To a solution of t-BuONa (1.7 g, 18.591 mmol, 3.0 equiv), 6-bromo-8-fluoro-1H-quinolin-2-one (D229, 1.5 g, 6.197 mmol, 1.0 equiv) and tert-butyl (2R,6S)-2,6-dimethylpiperazine-1-carboxylate (1.9 g, 9.296 mmol, 1.5 equiv) in dioxane (8.0 mL) were added Q-Phos (11.8 mg, 1.859 mmol, 0.3 equiv) and Pd₂(dba)₃ (11.8 mg, 0.619 mmol, 0.1 equiv). After stirring for 2 h at 100° C. under a nitrogen atmosphere, the resulting mixture was concentrated under reduced pressure. The residue was purified by silica gel column chromatography and eluted with PE/EA (1:2) to afford tert-butyl (2R,6S)-4-(8-fluoro-2-oxo-1H-quinolin-6-yl)-2,6-dimethylpiperazine-1-carboxylate (D230, 1.9 g). LCMS: (ES, m/z): 376 [M+H]⁺.

### Synthesis of Intermediate D231

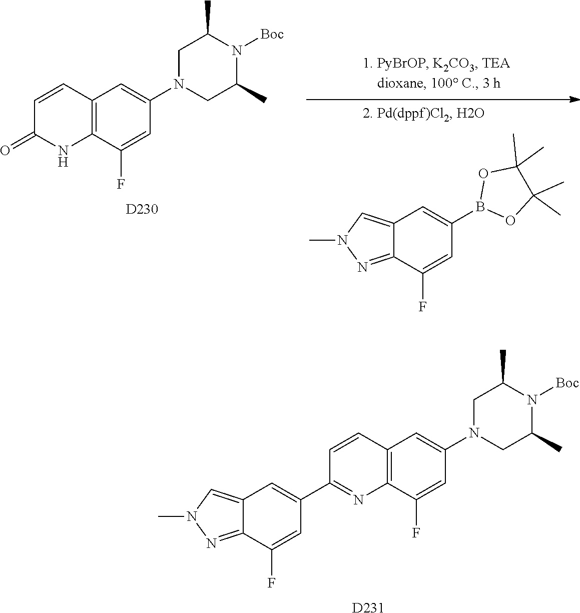

To a solution of Intermediate D230 (300.0 mg, 0.799 mmol, 1.0 equiv) in dioxane (10 mL) and TEA (242.5 mg, 2.397 mmol, 3.0 equiv) were added K₂CO₃ (331.3 mg, 2.397 mmol, 3 equiv) and PyBrOP (931.27 mg, 1.998 mmol, 2.5 equiv), which was stirred overnight at 100° C. under a nitrogen atmosphere. Then 7-fluoro-2-methyl-5-(4,4,5,5-tetramethyl-1,3,2-dioxaborolan-2-yl) indazole (330.9 mg, 1.199 mmol, 1.5 equiv), H₂O (1.0 mL) and Pd(dppf)Cl₂ (58.4 mg, 0.080 mmol, 0.1 equiv) were added, and the resulting mixture was stirred for an additional 1 h at 100° C. and subsequently concentrated off under reduced pressure. The residue was purified by silica gel column chromatography and eluted with PE/EA (1:1) to afford tert-butyl (2R,6S)-4-[8-fluoro-2-(7-fluoro-2-methylindazol-5-yl)quinolin-6-yl]-2,6-dimethylpiperazine-1-carboxylate (D231, 170.0 mg). LCMS: (ES, m/z): 508 [M+H]⁺.

### Synthesis of Compound 758

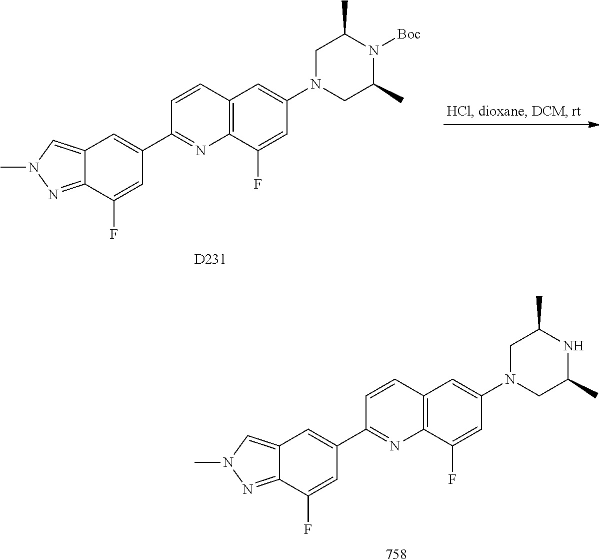

To a stirred mixture of Intermediate D231 (290.0 mg, 0.571 mmol, 1.0 equiv) was added HCl-dioxane (2.0 mL) and DCM (4.0 mL) dropwise at room temperature. The resulting mixture was stirred for 2 h and then concentrated under reduced pressure. The crude product was purified by Prep-HPLC following Condition 8, Gradient 3 as previously described to afford 6-[(3R,5S)-3,5-dimethylpiperazin-1-yl]-8-fluoro-2-(7-fluoro-2-methylindazol-5-yl)quinoline (Compound 758, 68.8 mg). Absolute stereochemistry of the compound was arbitrarily assigned. LCMS: (ES, m/z): 408 [M+H]⁺. ¹H NMR: (400 MHz, DMSO-d₆) δ 9.71 (d, J=10.4 Hz, 1H), 9.22 (d, J=11.0 Hz, 1H), 8.64 (d, J=2.8 Hz, 1H), 8.45 (d, J=1.2 Hz, 1H), 8.27 (dd, J=8.9, 1.6 Hz, 1H), 8.22 (d, J=8.9 Hz, 1H), 7.99 (dd, J=13.6, 1.3 Hz, 1H), 7.63 (dd, J=14.1, 2.6 Hz, 1H), 7.19 (d, J=2.5 Hz, 1H), 4.24 (s, 3H), 4.06 (dd, J=13.2, 3.1 Hz, 2H), 3.39 (d, J=9.8 Hz, 2H), 2.91 (dd, J=13.3, 11.2 Hz, 2H), 1.36 (d, J=6.5 Hz, 6H).

### Example 282: Synthesis of 763

### Synthesis of Intermediate D230

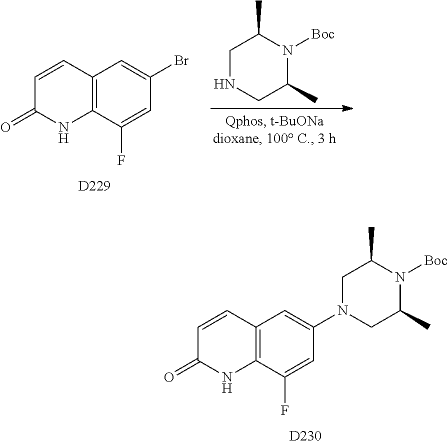

To a solution of t-BuONa (1.7 g, 18.591 mmol, 3.0 equiv), 6-bromo-8-fluoro-1H-quinolin-2-one (D229, 1.5 g, 6.197 mmol, 1.0 equiv) and tert-butyl (2R,6S)-2,6-dimethylpiperazine-1-carboxylate (1.9 g, 9.296 mmol, 1.5 equiv) in dioxane (8.0 mL) were added Q-Phos (11.8 mg, 1.859 mmol 0.3 equiv) and Pd₂(dba)₃ (11.8 mg, 0.618 mmol, 0.1 equiv). After stirring for 3 h at 100° C. under a nitrogen atmosphere, the resulting mixture was concentrated under reduced pressure. The residue was purified by silica gel column chromatography and eluted with PE/EA (1:2) to afford tert-butyl (2R,6S)-4-(8-fluoro-2-oxo-1H-quinolin-6-yl)-2,6-dimethylpiperazine-1-carboxylate (D230, 1.9 g). LCMS: (ES, m/z): 376 [M+H]⁺.

### Synthesis of Intermediate D232

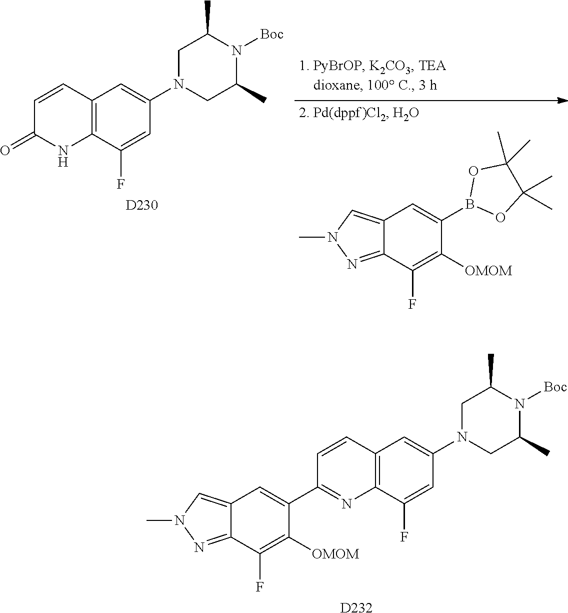

To a solution of Intermediate D230 (300.0 mg, 0.799 mmol, 1.0 equiv) in dioxane (10 mL) and TEA (242.5 mg, 2.397 mmol, 3.0 equiv) were added K₂CO₃ (331.3 mg, 2.397 mmol, 3.0 equiv) and PyBrOP (931.2 mg, 1.998 mmol, 2.5 equiv), which was stirred for 3 h at 100° C. under a nitrogen atmosphere. Then 6-(methoxymethoxy)-2-methyl-5-(4,4,5,5-tetramethyl-1,3,2-dioxaborolan-2-yl) indazole (381.3 mg, 1.199 mmol, 1.5 equiv), H₂O (1 mL) and Pd(dppf)Cl₂ (58.4 mg, 0.080 mmol, 0.1 equiv) were added. And the resulting mixture was stirred for 3 h at 100° C. and subsequently concentrated under reduced pressure. The residue was purified by silica gel column chromatography and eluted with PE/EA (1:1) to afford tert-butyl (2R,6S)-4-{8-fluoro-2-[7-fluoro-6-(methoxymethoxy)-2-methylindazol-5-yl]quinolin-6-yl}-2,6-dimethylpiperazine-1-carboxylate (D232, 200.0 mg, 44.09%). LCMS: (ES, m/z): 568 [M+H]⁺.

### Synthesis of Compound 763

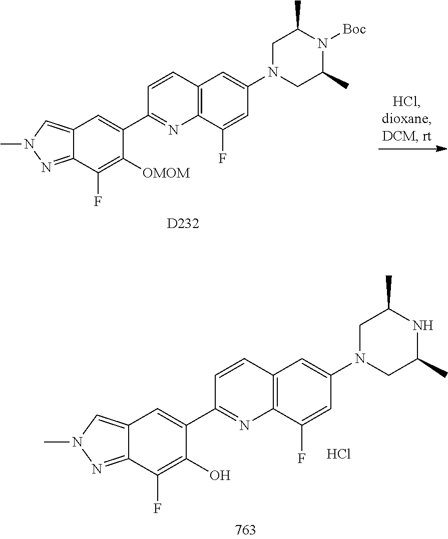

To a stirred solution of Intermediate D232 (290 mg, 0.511 mmol, 1.0 equiv) was added HCl-dioxane (2 mL, 4M) and DCM (4 mL) dropwise at room temperature. The resulting mixture was stirred for 2 h and subsequently concentrated under reduced pressure. The crude product was purified by Prep-HPLC following Condition 8, Gradient 3 as previously described to afford 5-{6-[(3R,5S)-3,5-dimethylpiperazin-1-yl]-8-fluoroquinolin-2-yl}-7-fluoro-2-methylindazol-6-ol hydrochloride (Compound 763, 32.6 mg). Absolute stereochemistry of the compound was arbitrarily assigned. LCMS: (ES, m/z): 424 [M+H]⁺. ¹H NMR: (400 MHz, DMSO-d₆) δ 14.55 (s, 1H), 9.48 (d, J=10.4 Hz, 1H), 9.00 (d, J=10.5 Hz, 1H), 8.57-8.50 (m, 2H), 8.44 (d, J=9.1 Hz, 1H), 8.39 (dd, J=9.2, 1.6 Hz, 1H), 7.74 (dd, J=13.9, 2.5 Hz, 1H), 7.26 (d, J=2.5 Hz, 1H), 4.18 (s, 3H), 4.11 (d, J=13.1 Hz, 2H), 2.91 (dd, J=13.4, 11.3 Hz, 2H), 1.35 (d, J=6.5 Hz, 6H).

### Example 283: Synthesis of Compound 768

### Synthesis of Intermediate D230

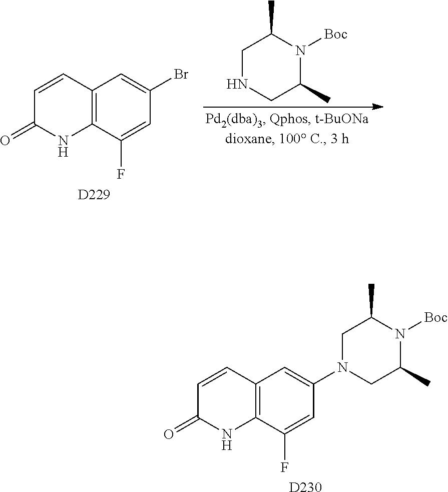

To a solution of t-BuONa (1.7 g, 18.591 mmol, 3.0 equiv), 6-bromo-8-fluoro-1H-quinolin-2-one (D229, 1.5 g, 6.197 mmol, 1.0 equiv) and tert-butyl (2R,6S)-2,6-dimethylpiperazine-1-carboxylate (1.9 g, 9.296 mmol, 1.5 equiv) in dioxane (8.0 mL) were added Q-Phos (11.8 mg, 1.859 mmol, 0.3 equiv) and Pd₂(dba)₃ (11.8 mg, 0.619 mmol, 0.1 equiv). After stirring for 2 h at 100° C. under a nitrogen atmosphere, the resulting mixture was concentrated under reduced pressure. The residue was purified by silica gel column chromatography and eluted with PE/EA (1:2) to afford tert-butyl (2R,6S)-4-(8-fluoro-2-oxo-1H-quinolin-6-yl)-2,6-dimethylpiperazine-1-carboxylate (D230, 1.9 g). LCMS: (ES, m/z): 376 [M+H]⁺.

### Synthesis of Intermediate D233

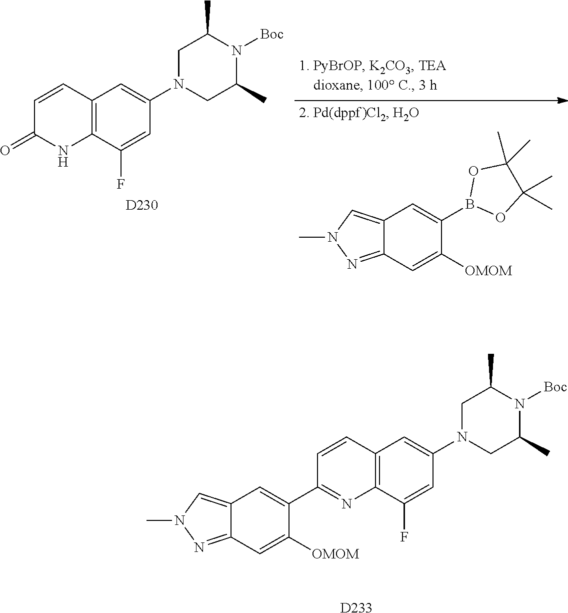

To a solution of Intermediate D230 (300.0 mg, 0.799 mmol, 1.0 equiv) in dioxane (10 mL) and TEA (242.5 mg, 2.397 mmol, 3.0 equiv) were added K₂CO₃ (331.3 mg, 2.397 mmol, 3.0 equiv) and PyBrOP (931.2 mg, 1.998 mmol, 2.5 equiv). The mixture was stirred for 3 h at 100° C. under a nitrogen atmosphere. Then 6-(methoxymethoxy)-2-methyl-5-(4,4,5,5-tetramethyl-1,3,2-dioxaborolan-2-yl) indazole (381.3 mg, 1.199 mmol, 1.5 equiv), H₂O (1 mL) and Pd(dppf)Cl₂ (58.4 mg, 0.080 mmol, 0.1 equiv) were added to the resulting mixture. After stirring for 2 h at 100° C., the reaction mixture was concentrated under reduced pressure. The residue was purified by silica gel column chromatography and eluted with PE/EA (1:1) to afford tert-butyl (2R,6S)-4-{8-fluoro-2-[6-(methoxymethoxy)-2-methylindazol-5-yl]quinolin-6-yl}-2,6-dimethylpiperazine-1-carboxylate (D233, 250 mg). LCMS: (ES, m/z): 550 [M+H]⁺.

### Synthesis of Compound 768

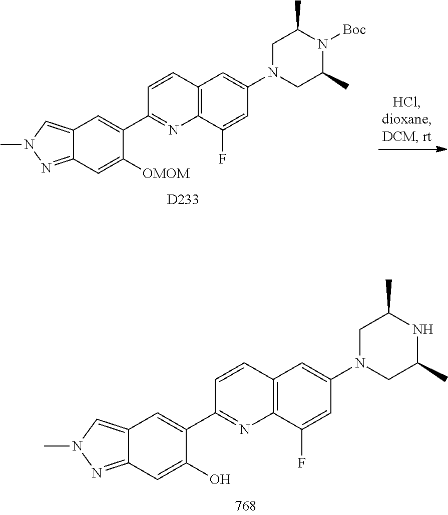

To a stirred solution/mixture of Intermediate D233 (200.0 mg, 0.364 mmol, 1 equiv) was added HCl-dioxane (2 mL, 4M) and DCM (4 mL) dropwise at room temperature. The resulting mixture was stirred for 2 h and subsequently concentrated under reduced pressure. The crude product was purified by Prep-HPLC following Condition 8, Gradient 3 as previously described to afford5-{6-[(3R,5S)-3,5-dimethylpiperazin-1-yl]-8-fluoroquinolin-2-yl}-2-methylindazol-6-ol hydrochloride (Compound 768, 68.2 mg). Absolute stereochemistry of the compound was arbitrarily assigned. LCMS: (ES, m/z): 442 [M+H]⁺. ¹H NMR: (400 MHz, DMSO-d₆) δ 8.65 (s, 1H), 8.42-8.41 (m, 2H), 8.36 (d, J=9.0 Hz, 1H), 7.70 (dd, J=13.8, 2.5 Hz, 1H), 7.24 (d, J=2.5 Hz, 1H), 6.90 (d, J=2.6 Hz, 1H), 4.13 (s, 3H), 4.13-4.09 (m, 2H), 3.45-3.44 (m, 2H), 2.84 (d, J=13.0 Hz, 2H), 1.31 (d, J=6.5 Hz, 6H).

### Example 284: Synthesis of Compound 773

### Synthesis of Intermediate D234

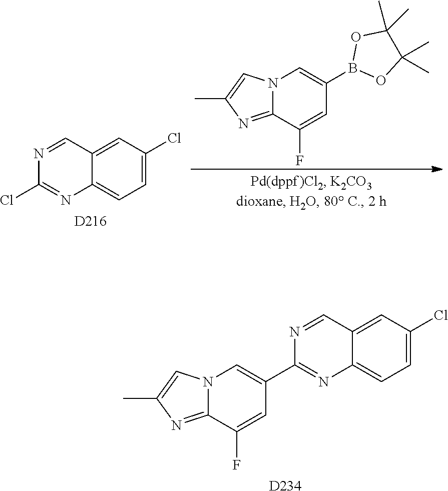

To a stirred solution of 2,6-dichloroquinazoline (D216, 100 mg, 0.502 mmol, 1 equiv), K₂CO₃ (209 mg, 1.51 mmol, 3 equiv) and 8-fluoro-2-methyl-6-(4,4,5,5-tetramethyl-1,3,2-dioxaborolan-2-yl)imidazo[1,2-a]pyridine (208 mg, 0.753 mmol, 1.5 equiv) in dioxane (2 mL) were added H₂O (0.2 mL) and Pd(dppf)Cl₂ (37 mg, 0.05 mmol, 0.1 equiv) at room temperature under nitrogen atmosphere. The resulting mixture was stirred for 2 h at 80° C. under nitrogen atmosphere and allowed to cool to room temperature. The reaction was quenched by the addition of water (30 mL) at room temperature and then extracted with EtOAc (3×10 mL). The combined organic layers were washed with brine (1×10 mL), dried over anhydrous Na₂SO₄, and the filtrate was concentrated under reduced pressure. The residue was purified by silica gel column chromatography and eluted with PE/THE (1:3) to afford 6-chloro-2-{8-fluoro-2-methylimidazo[1,2-a]pyridin-6-yl}quinazoline (D234, 60 mg). LCMS: (ES, m/z): 313 [M+H]⁺.

### Synthesis of Intermediate D235

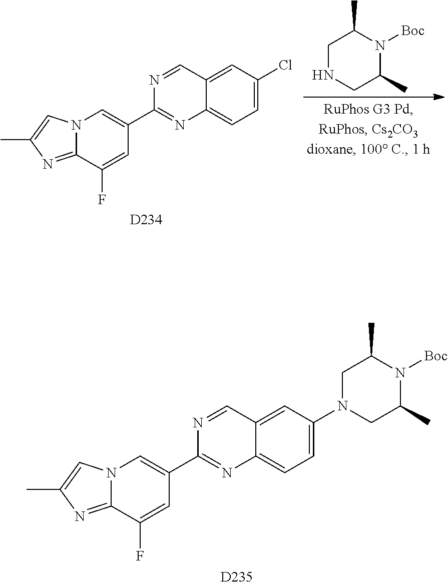

To a stirred solution of Intermediate D234 (60 mg, 0.192 mmol, 1 equiv), Cs₂CO₃ (125 mg, 0.384 mmol, 2 equiv) and tert-butyl (2R,6S)-2,6-dimethylpiperazine-1-carboxylate (82 mg, 0.384 mmol, 2 equiv) in dioxane (3 mL) were added RuPhos (18 mg, 0.038 mmol, 0.2 equiv) and RuPhos Palladacycle Gen.3 (16 mg, 0.019 mmol, 0.1 equiv) at room temperature under nitrogen atmosphere. The resulting mixture was stirred for 1 h at 100° C. under nitrogen atmosphere, allowed to cool to room temperature, quenched by the addition of water (50 mL) at room temperature, and then extracted with CH₂Cl₂ (3×10 mL). The combined organic layers were washed with brine (1×10 mL), dried over anhydrous Na₂SO₄, and the filtrate was concentrated under reduced pressure. The residue was purified by silica gel column chromatography and eluted with PE/THE (1:1) to afford tert-butyl (2R,6S)-4-(2-{8-fluoro-2-methylimidazo[1,2-a]pyridin-6-yl}quinazolin-6-yl)-2,6-dimethylpiperazine-1-carboxylate (D235, 63 mg). LCMS: (ES, m/z): 491 [M+H]⁺.

### Synthesis of Compound 773

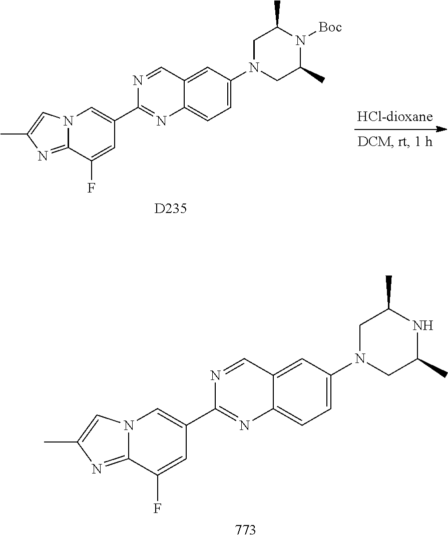

A solution of Intermediate D235 (63 mg, 0.128 mmol, 1 equiv) and 4 M HCl (gas) in 1,4-dioxane (0.5 mL) in DCM (1 mL) was stirred for 1 h at room temperature and subsequently concentrated under reduced pressure. The residue was purified by reversed-phase flash chromatography following Condition 10, Gradient 20 as previously described afford 6-[(3R,5S)-3,5-dimethylpiperazin-1-yl]-2-{8-fluoro-2-methylimidazo[1,2-a]pyridin-6-yl}quinazoline (Compound 773, 28 mg). Absolute stereochemistry of the compound was arbitrarily assigned. LCMS: (ES, m/z): 391 [M+H]⁺. ¹H NMR: (300 MHz, DMSO-d₆) δ 9.49-9.42 (m, 2H), 8.03 (dd, J=3.3, 1.0 Hz, 1H), 8.00-7.90 (m, 2H), 7.88 (d, J=9.4 Hz, 2H), 7.35 (d, J=2.5 Hz, 1H), 3.80 (d, J=10.5 Hz, 2H), 2.90 (s, 2H), 2.42-2.32 (m, 3H), 2.29 (d, J=10.7 Hz, 2H), 1.08 (d, J=6.2 Hz, 6H).

### Example 285: Synthesis of Compound 779

### Synthesis of Intermediate D113

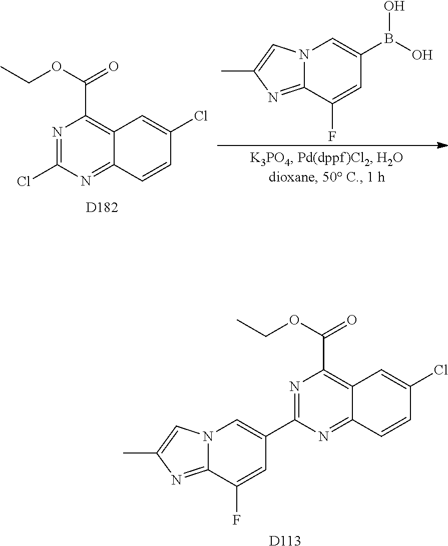

To a solution of ethyl 2,6-dichloroquinazoline-4-carboxylate (D182, 500.0 mg, 1.844 mmol, 1.0 equiv) and 8-fluoro-2-methylimidazo[1,2-a]pyridin-6-ylboronic acid (643.9 mg, 3.319 mmol, 1.8 equiv) in dioxane (8 mL) and H₂O (1.5 mL) were added K₃PO₄ (782.9 mg, 3.688 mmol, 2.0 equiv) and Pd(dppf)Cl₂ (134.9 mg, 0.184 mmol, 0.1 equiv). After stirring for 2 h at 80° C. under a nitrogen atmosphere, the resulting mixture was concentrated under reduced pressure. The residue was purified by silica gel column chromatography and eluted with PE/EA (1:1) to afford ethyl 6-chloro-2-{8-fluoro-2-methylimidazo[1,2-a]pyridin-6-yl}quinazoline-4-carboxylate (D113, 540.0 mg, 76.09%) as a yellow solid. LCMS: (ES, m/z): 385 [M+H]⁺.

### Synthesis of Intermediate D236

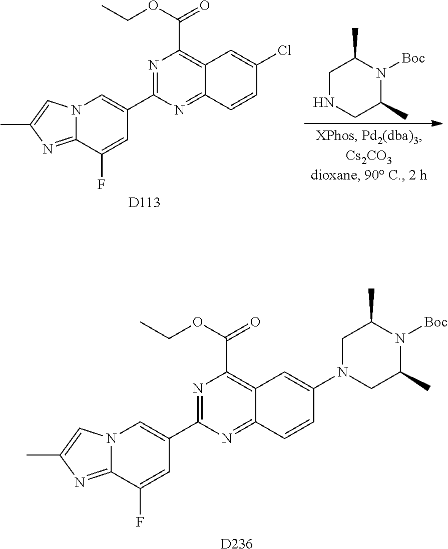

To a solution of Cs₂CO₃ (863.6 mg, 2.650 mmol, 2.0 equiv), Intermediate D113 (510.0 mg, 1.325 mmol, 1.0 equiv) and tert-butyl (2R,6S)-2,6-dimethylpiperazine-1-carboxylate (426.0 mg, 1.987 mmol, 1.5 equiv) in dioxane (10 mL) were added Pd₂(dba)₃ (121.3 mg, 0.133 mmol, 0.1 equiv) and XPhos (126.3 mg, 0.265 mmol, 0.2 equiv). After stirring for 2 h at 80° C. under a nitrogen atmosphere, the resulting mixture was concentrated under reduced pressure. The residue was purified by silica gel column chromatography and eluted with PE/EA (1:1) to afford ethyl 6-[(3R,5S)-4-(tert-butoxycarbonyl)-3,5-dimethylpiperazin-1-yl]-2-{8-fluoro-2-methylimidazo[1,2-a]pyridin-6-yl}quinazoline-4-carboxylate (D236, 600.0 mg). LCMS: (ES, m/z): 563 [M+H]⁺.

### Synthesis of Intermediate D237

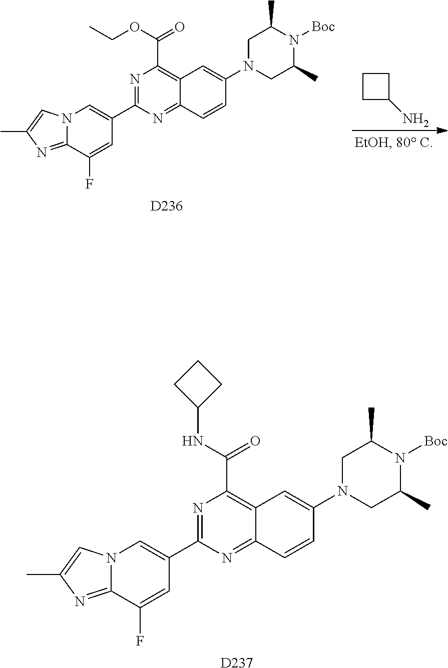 [SMILES: CCOC(=O)c1nc(-c2cc(F)c3nc(C)cn3c2)nc2ccc(N3C[C@@H](C)N(C(=O)OC(C)(C)C)[C@@H](C)C3)cc12 ; NC1CCC1 ; Cc1cn2cc(-c3nc(C(=O)NC4CCC4)c4cc(N5C[C@@H](C)N(C(=O)OC(C)(C)C)[C@@H](C)C5)ccc4n3)cc(F)c2n1 ; "D236" ; "D237"]

Intermediate D236 (325.0 mg, 0.578 mmol, 1.0 equiv) was added to cyclobutylamine (2.2 mL) and EtOH (6.5 mL), stirred for 2 h at 80° C., and subsequently concentrated under reduced pressure. The residue was purified by silica gel column chromatography and eluted with PE/EA (1:2) to afford tert-butyl (2R,6S)-4-[4-(cyclobutylcarbamoyl)-2-{8-fluoro-2-methylimidazo[1,2-a]pyridin-6-yl}quinazolin-6-yl]-2,6-dimethylpiperazine-1-carboxylate (D237, 250 mg). LCMS: (ES, m/z): 588 [M+H]⁺.

### Synthesis of Compound 779

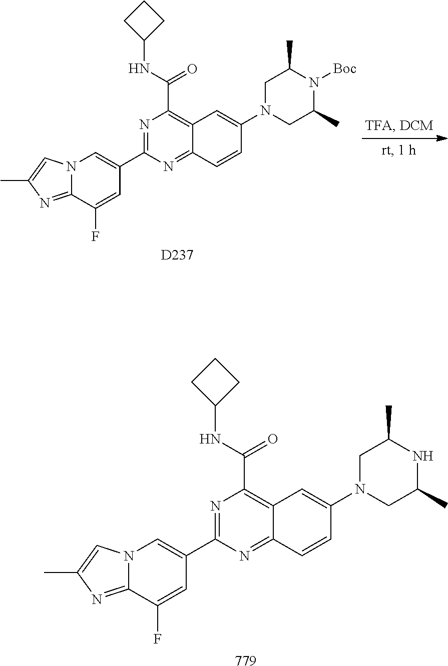

To a stirred mixture of Intermediate D237 (170.0 mg, 0.289 mmol, 1.0 equiv) was added HCl-dioxane (2 mL, 4M) and DCM (4 mL) dropwise at room temperature. The resulting mixture was stirred for 2 h and then concentrated under reduced pressure. The crude product was purified by Prep-HPLC following Condition 8, Gradient 3 as previously described to afford N-cyclobutyl-6-[(3R,5S)-3,5-dimethylpiperazin-1-yl]-2-{8-fluoro-2-methylimidazo[1,2-a]pyridin-6-yl}quinazoline-4-carboxamide (Compound 779, 64.2 mg). Absolute stereochemistry of the compound was arbitrarily assigned. LCMS: (ES, m/z): 488 [M+H]⁺. ¹H NMR: (400 MHz, DMSO-d₆) δ 9.51 (d, J=1.4 Hz, 1H), 9.30 (d, J=8.1 Hz, 1H), 8.12 (dd, J=12.5, 1.4 Hz, 1H), 8.07-8.02 (m, 1H), 8.02-7.90 (m, 3H), 4.58 (p, J=8.1 Hz, 1H), 3.74 (d, J=11.2 Hz, 2H), 2.89-2.88 (m, 2H), 2.43 (s, 3H), 2.32-2.18 (m, 7H), 1.83-1.70 (m, 2H), 1.08 (d, J=6.2 Hz, 6H).

### Example 286: Synthesis of Compound 752

### Synthesis of Intermediate D238

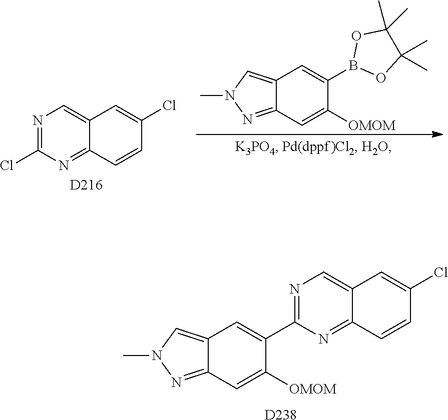

To a stirred solution of 2,6-dichloroquinazoline (D216, 350 mg, 1.759 mmol, 1 equiv) and 6-(methoxymethoxy)-2-methyl-5-(4,4,5,5-tetramethyl-1,3,2-dioxaborolan-2-yl) indazole (671 mg, 2.111 mmol, 1.2 equiv) in dioxane (10 mL) and H₂O (1 mL) were added K₃PO₄ (1119 mg, 5.277 mmol, 3 equiv) and Pd(dppf)Cl₂ (128 mg, 0.176 mmol, 0.1 equiv) at room temperature. The resulting mixture was stirred for 3 h at 80° C. under nitrogen atmosphere, quenched with water (10 mL) at room temperature, and then extracted with EtOAc (3×8 mL). The combined organic layers were washed with brine (1×5 mL), dried over anhydrous Na₂SO₄, and the filtrate was concentrated under reduced pressure. The residue was purified by silica gel column chromatography and eluted with PE/EA (1:9) to afford 6-chloro-2-[6-(methoxymethoxy)-2-methylindazol-5-yl]quinazoline (D238, 450 mg). LCMS: (ES, m/z): 355 [M+H]⁺.

### Synthesis of Intermediate D239

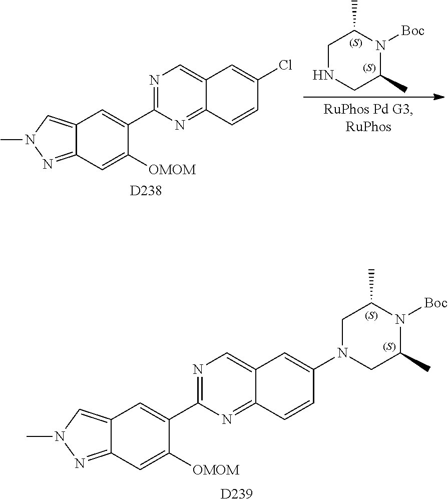

To a stirred solution of Intermediate D238 (85 mg, 0.395 mmol, 1 equiv) in dioxane (10 mL) were added RuPhos (37 mg, 0.079 mmol, 0.2 equiv), Cs₂CO₃ (385 mg, 1.185 mmol, 3 equiv) and Pd₂(dba)₃ (36 mg, 0.040 mmol, 0.1 equiv) at room temperature. The resulting mixture was stirred for 3 h at 80° C. under nitrogen atmosphere, quenched by the addition of water (10 mL) at room temperature, and then extracted with EtOAc (3×10 mL). The combined organic layers were washed with brine (1×10 mL), dried over anhydrous Na₂SO₄, and the filtrate was concentrated under reduced pressure. The residue was purified by silica gel column chromatography and eluted with PE/EA (5:95) to afford tert-butyl (2S,6S)-4-{2-[6-(methoxymethoxy)-2-methylindazol-5-yl]quinazolin-6-yl}-2,6-dimethylpiperazine-1-carboxylate (D239, 100 mg). LCMS: (ES, m/z): 533 [M+H]⁺.

### Synthesis of Compound 752

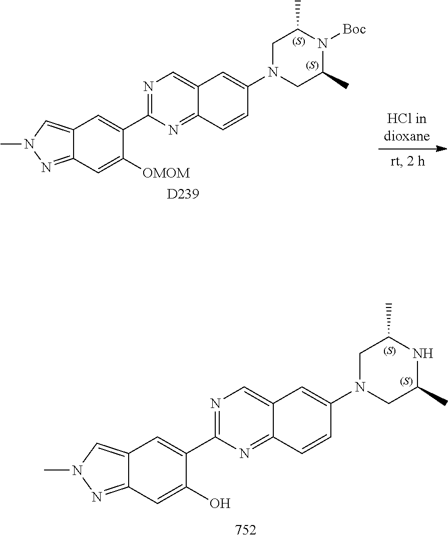

A mixture of Intermediate D239 (100 mg, 0.188 mmol, 1 equiv) and 4M HCl (gas) in 1,4-dioxane (5 mL) was stirred for 2 h at room temperature and then concentrated under reduced pressure. The residue was purified by reversed-phase flash chromatography following Condition 13, Gradient 1 as previously described to afford 5-{6-[(3S,5S)-3,5-dimethylpiperazin-1-yl]quinazolin-2-yl}-2-methylindazol-6-ol (Compound 752, 60 mg). Absolute stereochemistry of the compound was arbitrarily assigned. LCMS: (ES, m/z): 389 [M+H]⁺. ¹H NMR: (300 MHz, DMSO-d₆) δ 13.46 (s, 1H), 9.51 (s, 1H), 9.04 (s, 1H), 8.43 (s, 1H), 7.90 (d, J=1.6 Hz, 2H), 7.37 (t, J=1.7 Hz, 1H), 6.90 (s, 1H), 4.12 (s, 3H), 3.38 (dd, J=11.7, 3.3 Hz, 2H), 3.27 (td, J=6.2, 3.2 Hz, 2H), 3.03 (dd, J=11.7, 6.1 Hz, 2H), 1.15 (d, J=6.4 Hz, 6H).

### Example 287: Synthesis of Compound 753

### Synthesis of Intermediate D240

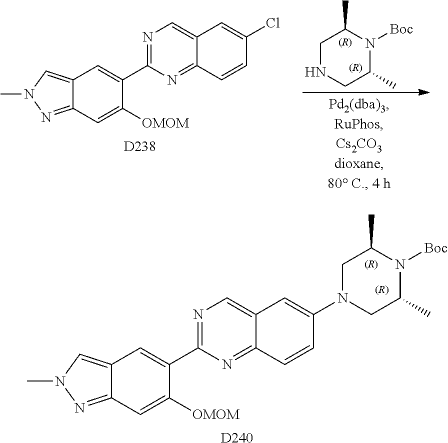

To a stirred mixture of 6-chloro-2-[6-(methoxymethoxy)-2-methylindazol-5-yl]quinazoline (D238, 150 mg, 0.423 mmol, 1 equiv) and tert-butyl (2R,6R)-2,6-dimethylpiperazine-1-carboxylate (109 mg, 0.509 mmol, 1.20 equiv) in dioxane (5 mL) were added RuPhos (39 mg, 0.084 mmol, 0.20 equiv), Pd₂(dba)₃ (39 mg, 0.043 mmol, 0.10 equiv) and Cs₂CO₃ (276 mg, 0.847 mmol, 2.00 equiv) at room temperature. The resulting mixture was stirred for 4 h at 80° C. under nitrogen atmosphere and then concentrated under reduced pressure. The residue was purified by silica gel column chromatography and eluted with PE/EA (1:1) to afford tert-butyl (2R,6R)-4-{2-[6-(methoxymethoxy)-2-methylindazol-5-yl]quinazolin-6-yl}-2,6-dimethylpiperazine-1-carboxylate (D240, 110 mg). LCMS: (ES, m/z): 533 [M+H]⁺.

### Synthesis of Compound 753

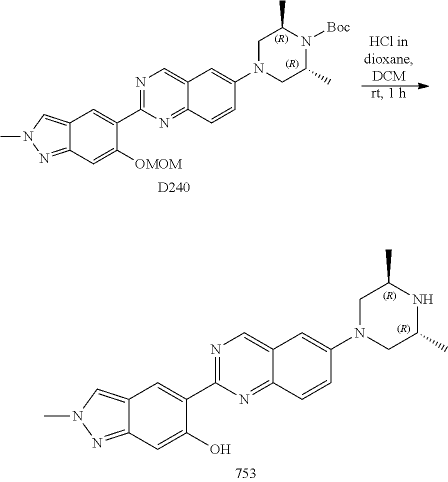

A solution of Intermediate D240 (110 mg, 0.207 mmol, 1 equiv) in DCM (2 mL) was treated with HCl (gas) in 1,4-dioxane (0.5 mL) for 1 h at room temperature and subsequently concentrated under reduced pressure. The residue was purified by reversed-phase flash chromatography following Condition 9, Gradient 5 as previously described to afford 5-{6-[(3R,5R)-3,5-dimethylpiperazin-1-yl]quinazolin-2-yl}-2-methylindazol-6-ol (Compound 753, 62.7 mg). Absolute stereochemistry of the compound was arbitrarily assigned. LCMS: (ES, m/z): 389 [M+H]⁺. RT=1.846 min on chiral-SFC. ¹H NMR: (300 MHz, DMSO-d₆) δ 13.45 (s, 1H), 9.52 (s, 1H), 9.04 (s, 1H), 8.43 (s, 1H), 7.92 (d, J=1.6 Hz, 2H), 7.39 (t, J=1.6 Hz, 1H), 6.90 (s, 1H), 4.12 (s, 3H), 3.51-3.40 (m, 2H), 3.39 (s, 2H), 3.07 (dd, J=11.9, 6.2 Hz, 2H), 1.17 (d, J=6.4 Hz, 6H).

### Example 288: Synthesis of Compound 785

### Synthesis of Intermediate D241

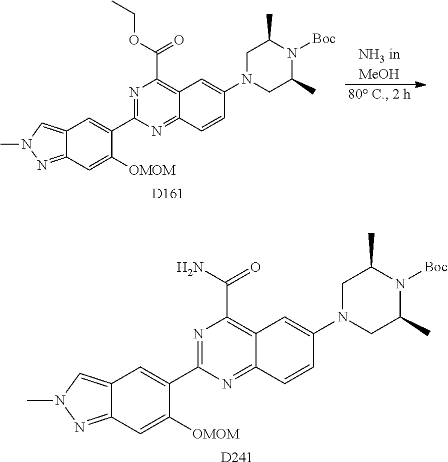

A mixture of ethyl 6-[(3R,5S)-4-(tert-butoxycarbonyl)-3,5-dimethylpiperazin-1-yl]-2-[6-(methoxymethoxy)-2-methylindazol-5-yl]quinazoline-4-carboxylate (D161, 70 mg, 0.116 mmol, 1 equiv) and NH₃(g) in MeOH (0.7 mL) was stirred for 2 h at 80° C. and subsequently concentrated under reduced pressure to afford tert-butyl (2R,6S)-4-{4-carbamoyl-2-[6-(methoxymethoxy)-2-methylindazol-5-yl]quinazolin-6-yl}-2,6-dimethylpiperazine-1-carboxylate (D241, 50 mg, crude), which was used directly in the next step without further purification. LCMS: (ES, m/z): 618 [M+H]⁺.

### Synthesis of Compound 785

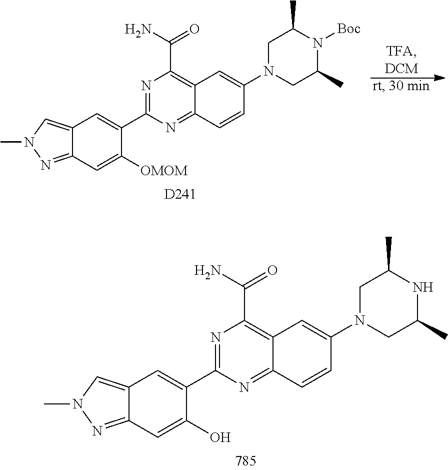

A solution of Intermediate D241 (50 mg, 0.087 mmol, 1 equiv) and TFA (0.15 mL) in DCM (2 mL) was stirred for 0.5 h at room temperature and then concentrated under reduced pressure. The residue was purified by reversed-phase flash chromatography following Condition 4, Gradient 7 as previously described to afford 6-((3R,5S)-3,5-dimethylpiperazin-1-yl)-2-(6-hydroxy-2-methyl-2H-indazol-5-yl)quinazoline-4-carboxamide 2,2,2-trifluoroacetate (Compound 785, 9 mg). Absolute stereochemistry of the compound was arbitrarily assigned. LCMS: (ES, m/z): 432 [M-TFA+H]⁺. ¹H NMR: (400 MHz, DMSO-d₆) δ 13.12 (s, 1H), 9.24 (s, 1H), 9.06 (s, 1H), 8.69 (s, 1H), 8.51 (d, J=10.6 Hz, 1H), 8.45 (s, 1H), 8.17-7.99 (m, 4H), 6.92 (s, 1H), 4.14 (s, 3H), 4.08 (d, J=12.7 Hz, 2H), 2.87-2.74 (m, 2H), 1.33 (d, J=6.5 Hz, 6H).

### Example 289: Synthesis of Compound 780

### Synthesis of Intermediate D234

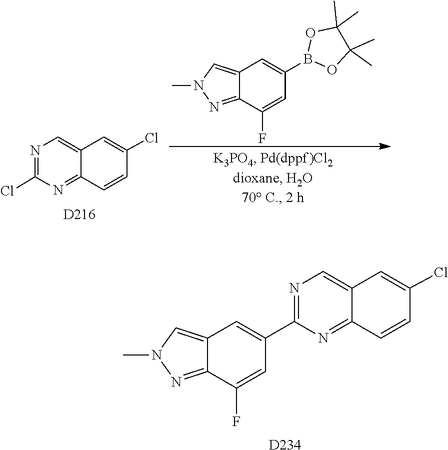

To a solution of 2,6-dichloroquinazoline (D216, 300 mg, 1.507 mmol, 1.0 equiv) and 7-fluoro-2-methyl-5-(4,4,5,5-tetramethyl-1,3,2-dioxaborolan-2-yl) indazole (457 mg, 1.658 mmol, 1.1 equiv) in dioxane (6.0 mL) and H₂O (1.0 mL) were added K₃PO₄ (959.85 mg, 4.521 mmol, 3.0 equiv) and Pd(dppf)Cl₂ (110.29 mg, 0.151 mmol, 0.1 equiv). After stirring for 2 h at 70° C. under a nitrogen atmosphere, the resulting mixture was concentrated under reduced pressure. The residue was purified by silica gel column chromatography and eluted with CH₂Cl₂/MeOH (20:1) to afford 2-(7-fluoro-2-methylindazol-5-yl)quinazoline (D234, 310 mg). LCMS: (ES, m/z): 313 [M+H]⁺.

### Synthesis of Intermediate D235

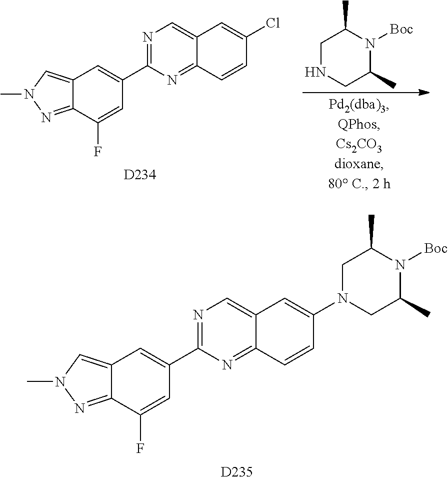

To a solution of Intermediate D234 (310 mg, 0.991 mmol, 1.0 equiv) and tert-butyl (2R,6S)-2,6-dimethylpiperazine-1-carboxylate (318 mg, 1.486 mmol, 1.5 equiv) in dioxane (6.0 mL) were added Cs₂CO₃ (645 mg, 1.982 mmol, 2 equiv), Pd₂(dba)₃ (90 mg, 0.099 mmol, 0.1 equiv) and Qphos (140 mg, 0.198 mmol, 0.2 equiv). After stirring for 2 h at 80° C. under a nitrogen atmosphere, the resulting mixture was concentrated under reduced pressure. The residue was purified by silica gel column chromatography and eluted with CH₂Cl₂/MeOH (20:1) to afford tert-butyl (2R,6S)-4-[2-(7-fluoro-2-methylindazol-5-yl)quinazolin-6-yl]-2,6-dimethylpiperazine-1-carboxylate (D235, 200.0 mg). LCMS: (ES, m/z): 491 [M+H]⁺.

### Synthesis of Compound 780

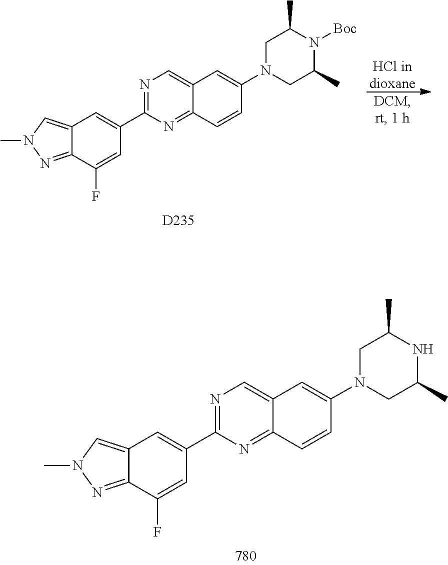

A mixture of Intermediate D235 (200 mg, 0.408 mmol, 1.0 equiv) and HCl (gas) in 1,4-dioxane (0.2 mL) in DCM (2.0 mL) was stirred for 1 h at room temperature and subsequently concentrated under reduced pressure. The crude product was purified by Prep-HPLC following Condition 19, Gradient 1 as previously described to afford 6-[(3R,5S)-3,5-dimethylpiperazin-1-yl]-2-(7-fluoro-2-methylindazol-5-yl)quinazoline (Compound 780, 67.3 mg). Absolute stereochemistry of the compound was arbitrarily assigned. LCMS: (ES, m/z): 391 [M+H]⁺. ¹H NMR: (400 MHz, DMSO-d₆) δ 9.44 (s, 1H), 8.75 (d, J=1.2 Hz, 1H), 8.65 (d, J=2.7 Hz, 1H), 8.10 (dd, J=13.5, 1.2 Hz, 1H), 7.95-7.84 (m, 2H), 7.33 (d, J=2.4 Hz, 1H), 4.24 (s, 3H), 3.83-3.75 (m, 2H), 2.90 (dd, J=9.7, 6.2 Hz, 2H), 2.30 (t, J=11.0 Hz, 2H), 1.08 (d, J=6.2 Hz, 6H).

### Example 290: Synthesis of Compound 787

### Synthesis of Intermediate D234

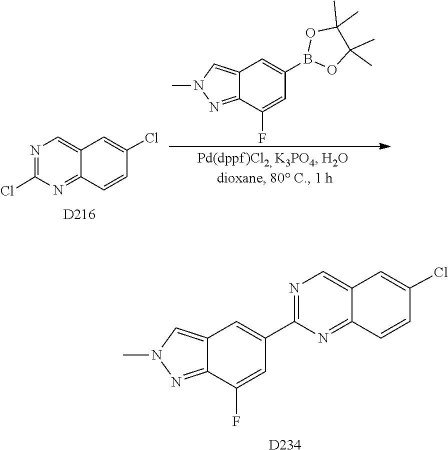

To a solution of 2,6-dichloroquinazoline (D216, 200 mg, 1.005 mmol, 1 equiv) and 7-fluoro-2-methyl-5-(4,4,5,5-tetramethyl-1,3,2-dioxaborolan-2-yl) indazole (278 mg, 1.005 mmol, 1 equiv) in dioxane (4 mL) and H₂O (1 mL) were added K₃PO₄ (640 mg, 3.015 mmol, 3 equiv) and Pd(dppf)Cl (82 mg, 0.100 mmol, 0.1 equiv). After stirring for 1 h at 80° C. under a nitrogen atmosphere, the resulting mixture was concentrated under reduced pressure. The residue was purified by reversed-phase flash chromatography following Condition 7, Gradient 6 as previously described and then concentrated under reduced pressure to afford 6-chloro-2-(7-fluoro-2-methylindazol-5-yl)quinazoline (D234, 130 mg). LCMS: (ES, m/z): 313 [M+H]⁺.

### Synthesis of Compound 787

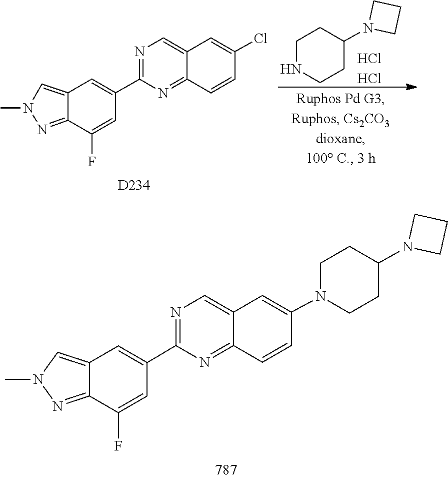

To a solution of Intermediate D234 (80 mg, 0.256 mmol, 1 equiv) and 4-(azetidin-1-yl)piperidine dihydrochloride (55 mg, 0.256 mmol, 1 equiv) in dioxane (2 mL) were added RuPhos (24 mg, 0.051 mmol, 0.2 equiv), Cs₂CO₃ (334 mg, 1.024 mmol, 4 equiv) and RuPhos Palladacycle Gen.3 (22 mg, 0.026 mmol, 0.1 equiv). After stirring for 3 h at 100° C. under a nitrogen atmosphere, the resulting mixture was concentrated under reduced pressure. The residue was purified by reversed-phase flash chromatography following Condition 10, Gradient 19 as previously described and then concentrated under reduced pressure to afford 6-[4-(azetidin-1-yl)piperidin-1-yl]-2-(7-fluoro-2-methylindazol-5-yl)quinazoline (Compound 787, 35 mg). LCMS: (ES, m/z): 417 [M+H]⁺. ¹H NMR: (300 MHz, DMSO-d₆) δ 9.43 (s, 1H), 8.75 (s, 1H), 8.65 (d, J=2.8 Hz, 1H), 8.09 (d, J=13.6 Hz, 1H), 7.87 (s, 2H), 7.34 (s, 1H), 4.24 (s, 3H), 3.76 (d, J=11.7 Hz, 2H), 3.14 (s, 4H), 2.99 (t, J=11.2 Hz, 2H), 2.28 (s, 1H), 1.94 (d, J=7.1 Hz, 2H), 1.76 (d, J=12.9 Hz, 2H), 1.28 (d, J=23.0 Hz, 2H).

### Example 291: Synthesis of Compound 781

### Synthesis of Intermediate D237

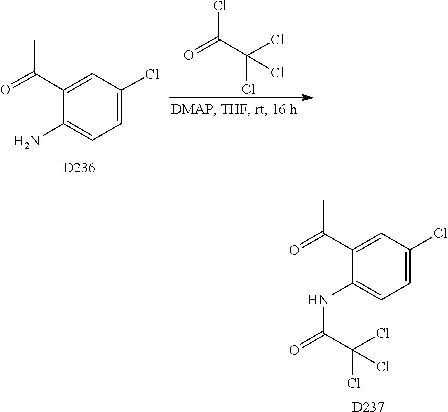

To a stirred solution of 1-(2-amino-5-chlorophenyl) ethanone (D236, 10 g, 58.959 mmol, 1 equiv) and trichloroacetyl chloride (10.72 g, 58.959 mmol, 1 equiv) in THF (200 mL) was added DMAP (10.80 g, 88.439 mmol, 1.5 equiv) at room temperature. The resulting mixture was stirred for 16 h at room temperature and subsequently concentrated under vacuum. The residue was purified by trituration with PE (50 mL) to afford N-(2-acetyl-4-chlorophenyl)-2,2,2-trichloroacetamide (D237, 12 g). LCMS: (ES, m/z): 314 [M+H]⁺.

### Synthesis of Intermediate D238

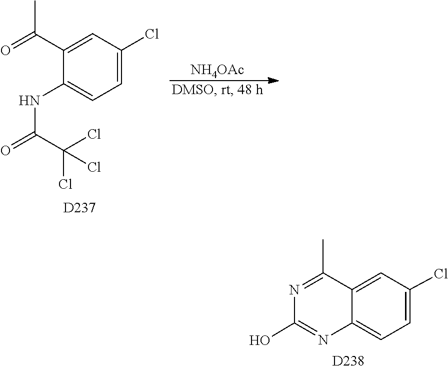

A solution of Intermediate D237 (12 g, 38.099 mmol, 1 equiv) and NH₄OAc (14.68 g, 190.495 mmol, 5 equiv) in DMSO (200 mL) was stirred for 36 h at room temperature and then quenched by water (800 mL) at room temperature. The precipitated solids were collected by filtration and washed with water (3×50 mL) to afford 6-chloro-4-methylquinazolin-2-ol (D238, 5.2 g, 70.13%). LCMS: (ES, m/z): 389 [M+H]⁺.

### Synthesis of Intermediate D239

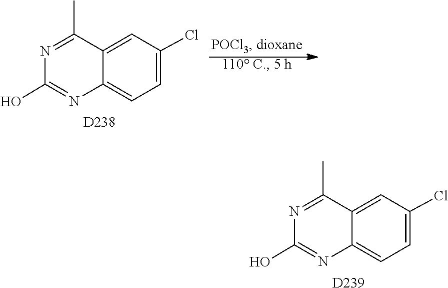

Intermediate D238 (5.2 g, 26.719 mmol, 1 equiv) and POCl₃ (20.48 g, 133.577 mmol, 5.00 equiv) were added to dioxane (100 mL), stirred for 3 h at 110° C., allowed to cool to room temperature. The reaction mixture was then quenched by the addition of water (100 mL) at 0° C. and extracted with EtOAc (3×50 mL). The combined organic layers were washed with brine (1×50 mL), dried over anhydrous Na₂SO₄, and the filtrate was concentrated under reduced pressure. The residue was purified by silica gel column chromatography and eluted with PE/EA (10:1) to afford 2,6-dichloro-4-methylquinazoline (D239, 2.1 g). LCMS: (ES, m/z): 213 [M+H]⁺.

### Synthesis of Intermediate D240

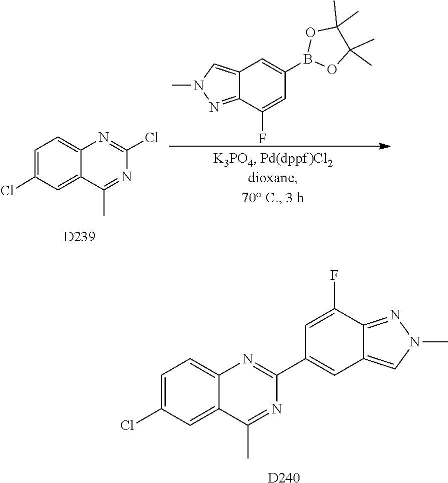

To a stirred solution of Intermediate D239 (500 mg, 2.347 mmol, 1 equiv), K₃PO₄ (1494 mg, 7.041 mmol, 3 equiv) and 7-fluoro-2-methyl-5-(4,4,5,5-tetramethyl-1,3,2-dioxaborolan-2-yl) indazole (777 mg, 2.816 mmol, 1.2 equiv) in dioxane (10 mL) were added H₂O (2 mL) and Pd(dppf)Cl₂CH₂Cl₂ (191 mg, 0.235 mmol, 0.1 equiv) at room temperature under nitrogen atmosphere. The resulting mixture was stirred for 3 h at 70° C. under nitrogen atmosphere, filtered, washed with EtOAc (3×30 mL), and concentrated under reduced pressure. The residue was purified by silica gel column chromatography and eluted with EA to afford 6-chloro-2-(7-fluoro-2-methylindazol-5-yl)-4-methylquinazoline (D240, 500 mg). LCMS: (ES, m/z): 327 [M+H]⁺.

### Synthesis of Compound 781

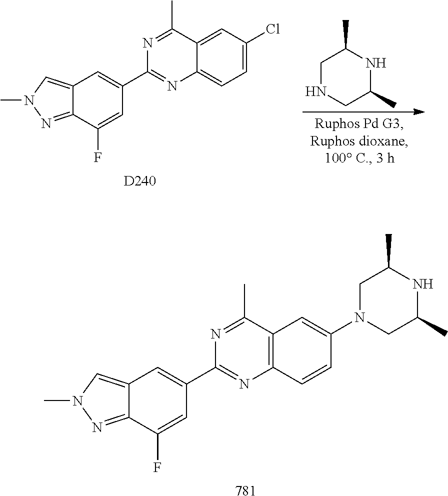

To a Intermediate D240 (200 mg, 0.612 mmol, 1 equiv) and (2R,6S)-2,6-dimethylpiperazine (104 mg, 0.918 mmol, 1.5 equiv) in dioxane (2 mL) were added Cs₂CO₃ (598 mg, 1.836 mmol, 3.0 equiv), RuPhos (57 mg, 0.122 mmol, 0.2 equiv) and RuPhos Palladacycle Gen.3 (51 mg, 0.061 mmol, 0.1 equiv). After stirring for 3 h at 100° C. under a nitrogen atmosphere, the resulting mixture was concentrated under reduced pressure. The residue was purified by reversed-phase flash chromatography following Condition 10, Gradient 18 as previously described to afford 6-[(3R,5S)-3,5-dimethylpiperazin-1-yl]-2-(7-fluoro-2-methylindazol-5-yl)-4-methylquinazoline (Compound 781, 45 mg). Absolute stereochemistry of the compound was arbitrarily assigned. LCMS: (ES, m/z): 405 [M+H]⁺. ¹H NMR: (300 MHz, DMSO-d6) δ 8.75 (s, 1H), 8.64 (d, J=2.8 Hz, 1H), 8.10 (dd, J=13.7, 1.3 Hz, 1H), 7.85 (d, J=1.9 Hz, 2H), 7.26 (s, 1H), 4.24 (s, 3H), 3.82 (d, J=11.5 Hz, 2H), 2.93 (s, 5H), 2.29 (t, J=11.0 Hz, 2H), 1.09 (d, J=6.2 Hz, 6H).

### Example 292: Synthesis of Compound 782

### Synthesis of Intermediate D242

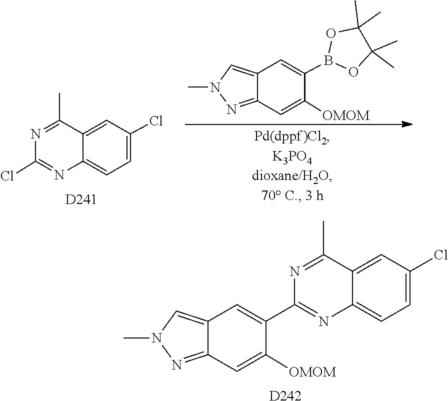

To a stirred solution of Intermediate D241 (250 mg, 1.173 mmol, 1 equiv) and 6-(methoxymethoxy)-2-methyl-5-(4,4,5,5-tetramethyl-1,3,2-dioxaborolan-2-yl) indazole (448 mg, 1.408 mmol, 1.2 equiv) in dioxane (5 mL) and H₂O (1 mL) were added K₃PO₄ (747 mg, 3.519 mmol, 3 equiv) and Pd(dppf)Cl₂ (90 mg, 0.117 mmol, 0.1 equiv) at room temperature under nitrogen atmosphere. The resulting mixture was stirred for 3 h at 70° C. under nitrogen atmosphere, allowed to cool to room temperature and then concentrated under reduced pressure. The residue was purified by silica gel column chromatography and eluted with PE/EA (1:1) to afford 6-chloro-2-[6-(methoxymethoxy)-2-methylindazol-5-yl]-4-methylquinazoline (D242, 210 mg). LCMS: (ES, m/z): 369 [M+H]⁺.

### Synthesis of Intermediate D243

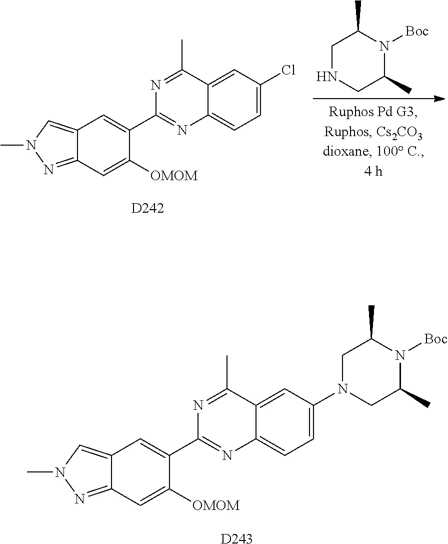

To a solution of Intermediate D242 (200 mg, 0.542 mmol, 1 equiv) and tert-butyl (2R,6S)-2,6-dimethylpiperazine-1-carboxylate (140 mg, 0.650 mmol, 1.2 equiv) in dioxane (3 mL) were added Cs₂CO₃ (530 mg, 1.626 mmol, 3.0 equiv), RuPhos (51 mg, 0.108 mmol, 0.2 equiv) and RuPhos Palladacycle Gen.3 (46 mg, 0.054 mmol, 0.1 equiv). After stirring for 4 h at 100° C. under a nitrogen atmosphere, the resulting mixture was concentrated under reduced pressure. The residue was purified by silica gel column chromatography and eluted with EA/MeOH (3:1) to afford tert-butyl (2R,6S)-4-{2-[6-(methoxymethoxy)-2-methylindazol-5-yl]-4-methylquinazolin-6-yl}-2,6-dimethylpiperazine-1-carboxylate (D243, 250 mg). LCMS: (ES, m/z): 547 [M+H]⁺.

### Synthesis of Compound 782

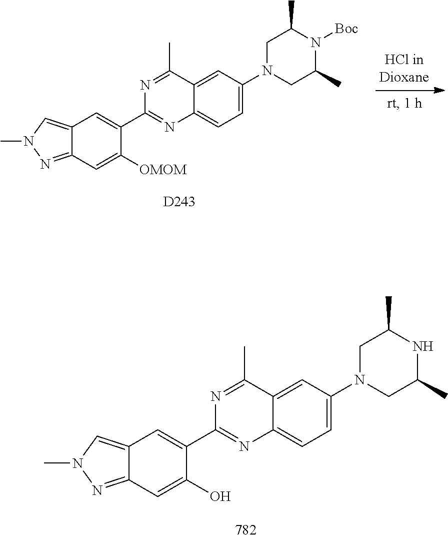

To a stirred solution of Intermediate D243 (190 mg, 0.348 mmol, 1 equiv) in DCM (2 mL) was added 4 M HCl (gas) in 1,4-dioxane (2 mL) dropwise at room temperature. The resulting mixture was stirred for 1 h at room temperature and subsequently concentrated under reduced pressure. The residue was purified by reversed-phase flash chromatography following Condition 4, Gradient 6 as previously described to afford 5-(6-((3S,5R)-3,5-dimethylpiperazin-1-yl)-4-methylquinazolin-2-yl)-2-methyl-2H-indazol-6-ol 2,2,2-trifluoroacetate (Compound 782, 38 mg). Absolute stereochemistry of the compound was arbitrarily assigned. LCMS: (ES, m/z): 403 [M+H]⁺. ¹H NMR: (300 MHz, DMSO-d₆) δ 9.14 (s, 1H), 9.06 (s, 1H), 8.56 (s, 1H), 8.45 (s, 1H), 7.96 (d, J=2.6 Hz, 2H), 7.47 (s, 1H), 6.89 (s, 1H), 4.20 (d, J=13.1 Hz, 2H), 4.13 (s, 3H), 3.48 (s, 2H), 3.01 (s, 3H), 2.82 (t, J=12.3 Hz, 2H), 1.34 (d, J=6.5 Hz, 6H).

### Example 293: Synthesis of Compound 783

### Synthesis of Intermediate D244

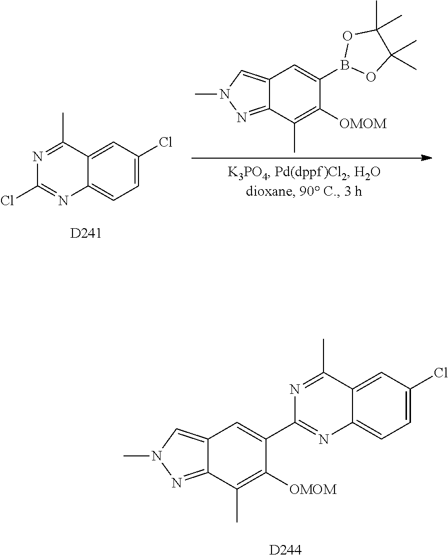

To a stirred solution of 2,6-dichloro-4-methylquinazoline (D241, 300 mg, 1.408 mmol, 1 equiv) and 6-(methoxymethoxy)-2,7-dimethyl-5-(4,4,5,5-tetramethyl-1,3,2-dioxaborolan-2-yl) indazole (561 mg, 1.690 mmol, 1.2 equiv) in dioxane (6 mL) and H₂O (0.6 mL) were added K₃PO₄ (896 mg, 4.224 mmol, 3 equiv) and Pd(dppf)Cl₂ (103 mg, 0.141 mmol, 0.1 equiv) at room temperature under nitrogen atmosphere. The resulting mixture was stirred for 3 h at 100° C. under nitrogen atmosphere and then concentrated under reduced pressure. The residue was purified by silica gel column chromatography and eluted with PE/EA (1:1) to afford 6-chloro-2-[6-(methoxymethoxy)-2,7-dimethylindazol-5-yl]-4-methylquinazoline (D244, 320 mg). LCMS: (ES, m/z): 383 [M+H]⁺.

### Synthesis of Intermediate D245

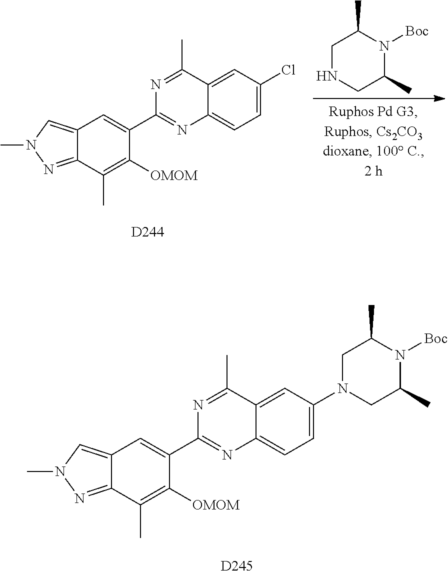

To a stirred solution of Intermediate D244 (320 mg, 0.836 mmol, 1 equiv) and tert-butyl (2R,6S)-2,6-dimethylpiperazine-1-carboxylate (268 mg, 1.254 mmol, 1.5 equiv) in dioxane (5 mL) were added RuPhos (78 mg, 0.167 mmol, 0.2 equiv) and RuPhos Palladacycle Gen.3 (69 mg, 0.084 mmol, 0.1 equiv) at room temperature under nitrogen atmosphere. The resulting mixture was stirred for 3 h at 100° C., allowed to cool to room temperature, filtered and then washed with EtOAc (3×100 mL). The filtrate was concentrated under reduced pressure. The residue was purified by silica gel column chromatography and eluted with EA to afford tert-butyl tert-butyl (2R,6S)-4-{2-[6-(methoxymethoxy)-2,7-dimethylindazol-5-yl]-4-methylquinazolin-6-yl}-2,6-dimethylpiperazine-1-carboxylate (D245, 290 mg). LCMS: (ES, m/z): 561 [M+H]⁺.

### Synthesis of Compound 783

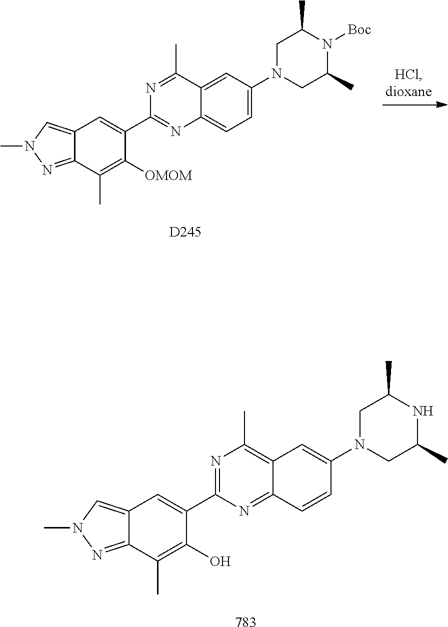

A solution of Intermediate D245 (290 mg, 0.517 mmol, 1 equiv) in 4 M HCl (gas) in 1,4-dioxane (3 mL) was stirred for 2 h at room temperature and subsequently concentrated under vacuum. The residue was purified by reversed-phase flash chromatography following Condition 10, Gradient 8 as previously described to afford 5-{6-[(3R,5S)-3,5-dimethylpiperazin-1-yl]-4-methylquinazolin-2-yl}-2,7-dimethylindazol-6-ol (Compound 783, 90 mg). Absolute stereochemistry of the compound was arbitrarily assigned. LCMS: (ES, m/z): 417 [M+H]⁺. ¹H NMR: (300 MHz, Chloroform-d) δ 14.09 (s, 1H), 9.00 (s, 1H), 7.95 (s, 1H), 7.87 (d, J=9.3 Hz, 1H), 7.67 (dd, J=9.3, 2.6 Hz, 1H), 7.17 (d, J=2.6 Hz, 1H), 4.23 (s, 3H), 3.74-3.64 (m, 2H), 3.13 (ddd, J=9.6, 6.3, 2.9 Hz, 2H), 2.99 (s, 3H), 2.61 (s, 3H), 2.46 (t, J=11.1 Hz, 2H), 1.23 (d, J=6.3 Hz, 6H).

### Example 294: Synthesis of Compound 784

### Synthesis of Intermediate D246

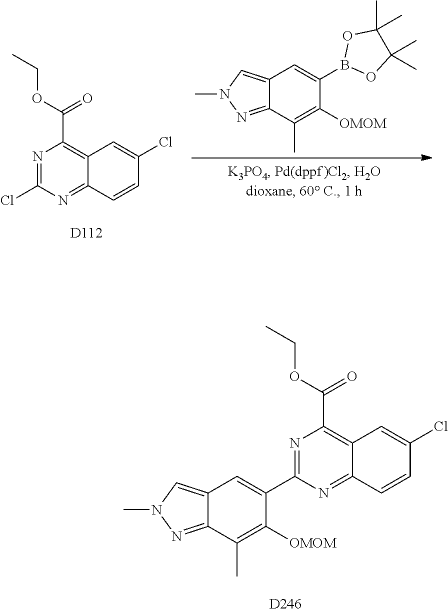

To a solution of ethyl 2,6-dichloroquinazoline-4-carboxylate (D112, 150 mg, 0.553 mmol, 1 equiv) in 1,4-dioxane (1.5 mL)/H₂O (0.15 mL) were added 6-(methoxymethoxy)-2,7-dimethyl-5-(4,4,5,5-tetramethyl-1,3,2-dioxaborolan-2-yl) indazole (183.8 mg, 0.553 mmol, 1 equiv), K₃PO₄ (234.9 mg, 1.106 mmol, 2 equiv) and Pd(dppf)Cl₂·CH₂Cl₂ (45.0 mg, 0.055 mmol, 0.1 equiv). The reaction mixture was stirred for 1 h at 60° C. under nitrogen atmosphere, then diluted with water (15 mL), and extracted with DCM (2×20 mL). The combined organic layers were washed with water (2×30 mL), dried over anhydrous Na₂SO₄, and the filtrate was concentrated under reduced pressure. The residue was purified by silica gel column chromatography and eluted with DCM/EA (1:1) to afford ethyl 6-chloro-2-[6-(methoxymethoxy)-2,7-dimethylindazol-5-yl]quinazoline-4-carboxylate (D246, 175 mg, 71.74%). LCMS: (ES, m/z): 441 [M+H]⁺.

### Synthesis of Intermediate D247

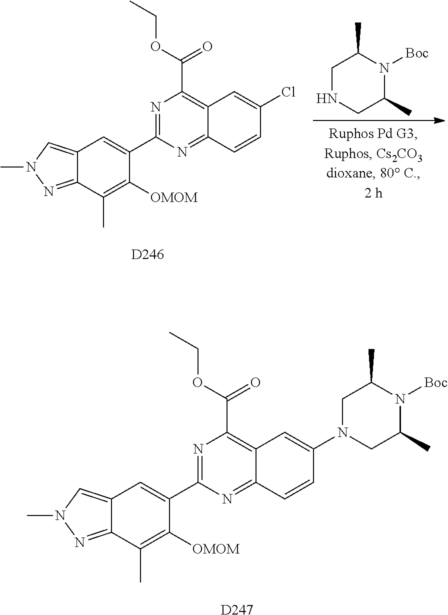

To a solution of Intermediate D246 (175 mg, 0.397 mmol, 1 equiv) in 1,4-dioxane (2 mL) were added tert-butyl (2R,6S)-2,6-dimethylpiperazine-1-carboxylate (102.1 mg, 0.476 mmol, 1.2 equiv), Cs₂CO₃ (258.6 mg, 0.794 mmol, 2 equiv), RuPhos (37.0 mg, 0.079 mmol, 0.2 equiv) and RuPhos Palladacycle Gen.3 (33.2 mg, 0.040 mmol, 0.1 equiv). The mixture was stirred for 2 h at 80° C. under nitrogen atmosphere, diluted with water (15 mL), and then extracted with DCM (2×20 mL). The combined organic layers were washed with water (2×30 mL), dried over anhydrous Na₂SO₄, and the filtrate was concentrated under reduced pressure. The residue was purified by silica gel column chromatography and eluted with PE/EA (1:9) to afford ethyl 6-[(3R,5S)-4-(tert-butoxycarbonyl)-3,5-dimethylpiperazin-1-yl]-2-[6-(methoxymethoxy)-2,7-dimethylindazol-5-yl]quinazoline-4-carboxylate (D247, 110 mg). LCMS: (ES, m/z): 619 [M+H]

### Synthesis of Intermediate D248

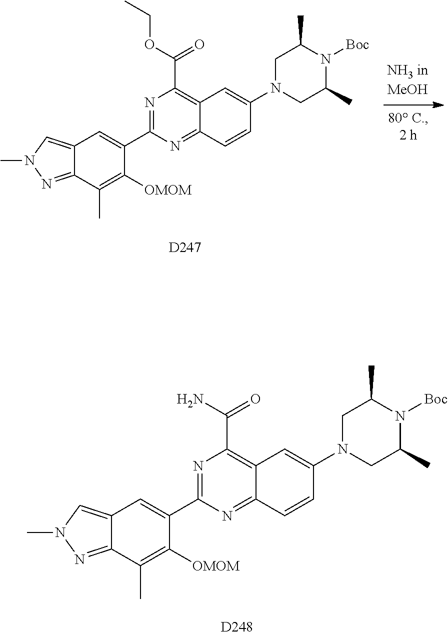

Intermediate D247 (110 mg, 0.178 mmol, 1 equiv) in a solution of NH₃ in MeOH (2 mL, 7 M) was stirred for 2 h at 80° C. and subsequently concentrated under reduced pressure. The residue was purified by silica gel column chromatography and eluted with PE/EA (1:9) to afford tert-butyl (2R,6S)-4-{4-carbamoyl-2-[6-(methoxymethoxy)-2,7-dimethylindazol-5-yl]quinazolin-6-yl}-2,6-dimethylpiperazine-1-carboxylate (D248, 90 mg). LCMS: (ES, m/z): 590 [M+H]⁺.

### Synthesis of Compound 784

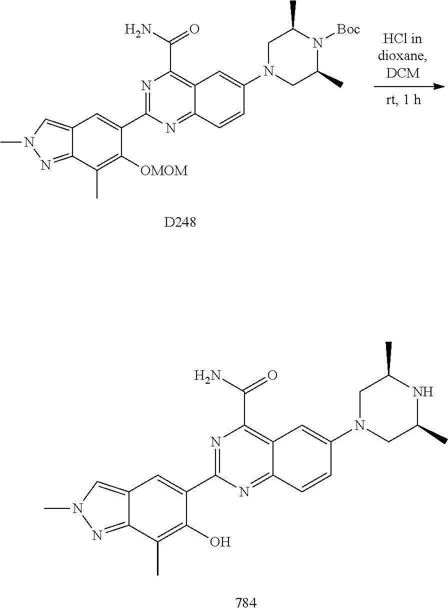

To a solution of Intermediate D248 (90 mg, 0.153 mmol, 1 equiv) in DCM (1 mL) was added HCl (gas) in 1,4-dioxane (0.45 mL, 4 M) for 1 h at room temperature. The resulting mixture was concentrated under reduced pressure, and the crude product was purified by Prep-HPLC following Condition 6, Gradient 11 as previously described to afford 6-[(3R,5S)-3,5-dimethylpiperazin-1-yl]-2-(6-hydroxy-2,7-dimethylindazol-5-yl)quinazoline-4-carboxamide (Compound 784, 15 mg). Absolute stereochemistry of the compound was arbitrarily assigned. LCMS: (ES, m/z): 446 [M+H]⁺. ¹H NMR: (400 MHz, DMSO-d₆) δ 13.37 (s, 1H), 9.05 (s, 1H), 8.62 (s, 1H), 8.40 (s, 1H), 8.06 (s, 1H), 7.99 (d, J=2.5 Hz, 2H), 7.98-7.91 (m, 1H), 4.16 (s, 3H), 3.74 (dd, J=11.8, 2.8 Hz, 2H), 2.95-2.85 (m, 2H), 2.41 (s, 3H), 2.37-2.23 (m, 3H), 1.24-1.23 (m, 1H), 1.08 (d, J=6.3 Hz, 6H).

### Example 295: Synthesis of Compound 786

### Synthesis of Intermediate D250

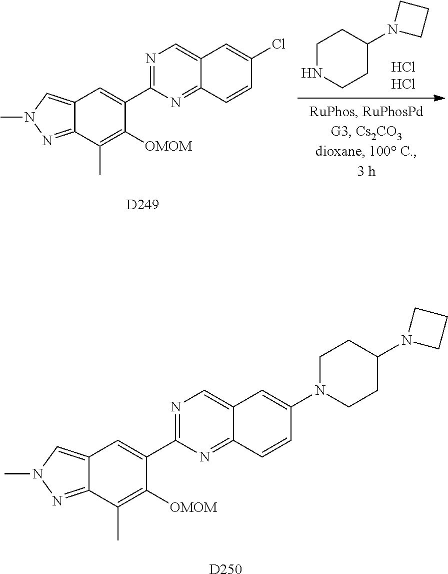

To a stirred solution of 6-chloro-2-[6-(methoxymethoxy)-2,7-dimethylindazol-5-yl]quinazoline (D249, 220 mg, 0.596 mmol, 1 equiv) and 4-(azetidin-1-yl)piperidine dihydrochloride (153 mg, 0.715 mmol, 1.2 equiv) in dioxane (5 mL) were added RuPhos (56 mg, 0.119 mmol, 0.2 equiv), RuPhos Palladacycle Gen.3 (29 mg, 0.060 mmol, 0.1 equiv) and Cs₂CO₃ (590 mg, 1.788 mmol, 3 equiv) at room temperature. The resulting mixture was stirred for 3 h at 100° C. under nitrogen atmosphere and then concentrated under reduced pressure. The residue was purified by silica gel column chromatography and eluted with CH₂Cl₂/MeOH (8:1) to afford 6-[4-(azetidin-1-yl)piperidin-1-yl]-2-[6-(methoxymethoxy)-2,7-dimethylindazol-5-yl]quinazoline) (D250). LCMS: (ES, m/z): 473 [M+H]⁺.

### Synthesis of Compound 786

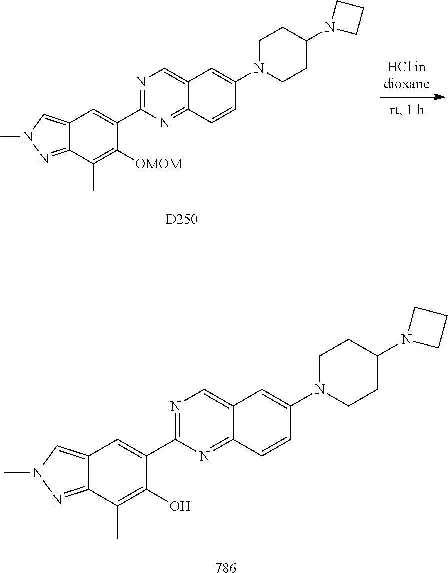

A solution of Intermediate D250 (160 mg, 0.339 mmol, 1 equiv) in 4 M HCl (gas) in 1,4-dioxane (5 mL) was stirred for 1 h at room temperature and then concentrated under reduced pressure. The residue was purified by reversed-phase flash chromatography following Condition 10, Gradient 10 as previously described to afford 5-{6-[4-(azetidin-1-yl)piperidin-1-yl]quinazolin-2-yl}-2,7-dimethylindazol-6-ol (Compound 786, 75 mg). LCMS: (ES, m/z): 429 [M+H]⁺. ¹H NMR: (300 MHz, Chloroform-d) δ 13.86 (s, 1H), 9.27 (s, 1H), 8.96 (s, 1H), 7.95 (s, 1H), 7.85 (d, J=9.3 Hz, 1H), 7.68 (dd, J=9.3, 2.7 Hz, 1H), 7.09 (d, J=2.6 Hz, 1H), 4.23 (s, 3H), 3.79 (d, J=12.5 Hz, 2H), 3.29 (d, J=7.7 Hz, 4H), 3.05-2.90 (m, 2H), 2.62 (s, 3H), 2.29 (s, 1H), 2.14 (t, J=7.1 Hz, 2H), 1.88 (d, J=12.8 Hz, 2H), 1.53 (d, J=11.4 Hz, 2H).

### Example 296: Synthesis of Compound 788

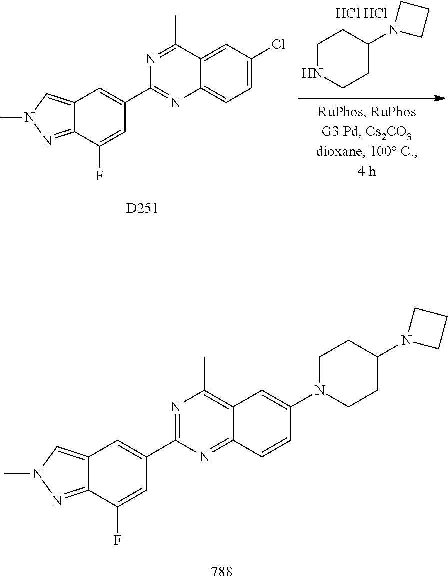

To a solution of 6-chloro-2-(7-fluoro-2-methylindazol-5-yl)-4-methylquinazoline (D251, 200.0 mg, 0.612 mmol, 1.0 equiv) and 4-(azetidin-1-yl)piperidine dihydrochloride (195 mg, 0.918 mmol, 1.5 equiv) in dioxane (5.0 mL) were added RuPhos Palladacycle Gen.3 (51 mg, 0.061 mmol, 0.1 equiv), RuPhos (28 mg, 0.061 mmol, 0.1 equiv) and Cs₂CO₃ (398 mg, 1.224 mmol, 2.0 equiv). After stirring for 4 h at 100° C. under a nitrogen atmosphere, the resulting mixture was allowed to cool to room temperature, and the filtrate was concentrated under reduced pressure. The residue was purified by Prep-HPLC following Condition 19, Gradient 1 as previously described to afford 6-[4-(azetidin-1-yl)piperidin-1-yl]-2-(7-fluoro-2-methylindazol-5-yl)-4-methylquinazoline (Compound 788, 84.7 mg). LCMS: (ES, m/z): 431 [M+H]⁺. ¹H NMR: (400 MHz, DMSO-d₆) δ 8.75 (s, 1H), 8.64 (d, J=2.8 Hz, 1H), 8.14-8.06 (m, 1H), 7.89-7.78 (m, 2H), 7.28 (d, J=2.5 Hz, 1H), 4.24 (s, 3H), 3.76 (d, J=12.6 Hz, 2H), 3.12 (t, J=6.8 Hz, 4H), 3.01 (t, J=10.5 Hz, 2H), 2.92 (s, 3H), 2.21 (d, J=5.1 Hz, 1H), 1.94 (q, J=6.8 Hz, 2H), 1.76 (d, J=12.3 Hz, 2H), 1.38-1.29 (m, 2H).

### Example 297: Synthesis of Compound 789

### Synthesis of Intermediate D253

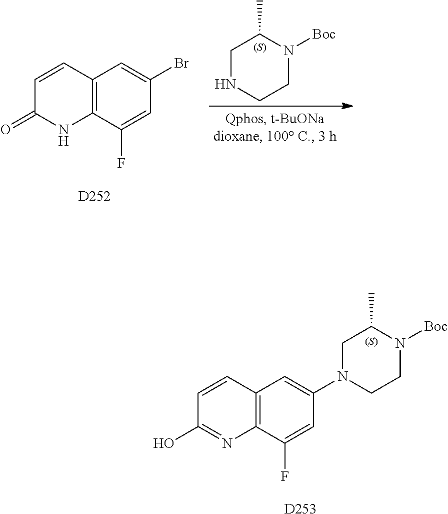

To a solution of t-BuONa (357.3 mg, 3.717 mmol, 3.0 equiv), 6-bromo-8-fluoro-1H-quinolin-2-one (D252, 300.0 mg, 1.239 mmol, 1.0 equiv) and tert-butyl (2S)-2-methylpiperazine-1-carboxylate (372.3 mg, 1.859 mmol, 1.5 equiv) in dioxane (8 mL) were added QPhos (11.9 mg, 0.372 mmol, 0.3 equiv) and Pd₂(dba)₃ (11.8 mg, 0.124 mmol, 0.1 equiv). After stirring for 2 h at 100° C. under a nitrogen atmosphere, the resulting mixture was concentrated under reduced pressure. The residue was purified by silica gel column chromatography and eluted with PE/EA (1:2) to afford tert-butyl (2S)-4-(8-fluoro-2-hydroxyquinolin-6-yl)-2-methylpiperazine-1-carboxylate (D253, 400 mg). LCMS: (ES, m/z): 362 [M+H]⁺.

### Synthesis of Intermediate D254

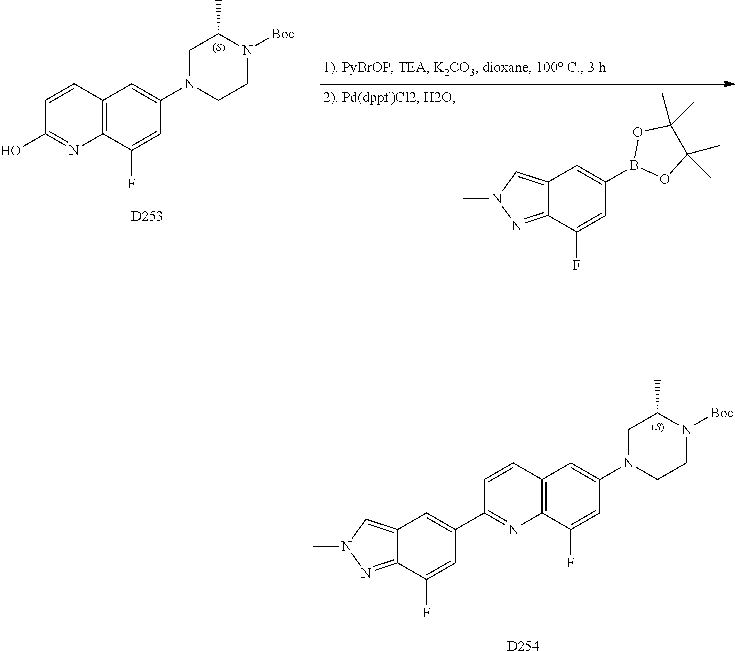

To a solution of Intermediate D253 (400.0 mg, 1.107 mmol, 1.0 equiv) and TEA (335.9 mg, 3.321 mmol, 3.0 equiv) in dioxane (10 mL) were added K₂CO₃ (611.8 mg, 4.428 mmol, 4.0 equiv) and PyBrOP (1289.8 mg, 2.768 mmol, 2.5 equiv). The reaction was stirred overnight at 100° C. under a nitrogen atmosphere. To the above mixture were added 7-fluoro-2-methyl-5-(4,4,5,5-tetramethyl-1,3,2-dioxaborolan-2-yl) indazole (458.4 mg, 1.660 mmol, 1.5 equiv), H₂O (1 mL) and Pd(dppf)Cl₂ (80.9 mg, 0.111 mmol, 0.1 equiv), which was stirred for an additional 2 h at 100° C. under a nitrogen atmosphere and subsequently concentrated under reduced pressure. The residue was purified by silica gel column chromatography and eluted with PE/EA (1:1) to afford tert-butyl (2S)-4-[8-fluoro-2-(7-fluoro-2-methylindazol-5-yl)quinolin-6-yl]-2-methylpiperazine-1-carboxylate (D254, 280.0 mg). LCMS: (ES, m/z): 494 [M+H]⁺.

### Synthesis of Compound 789

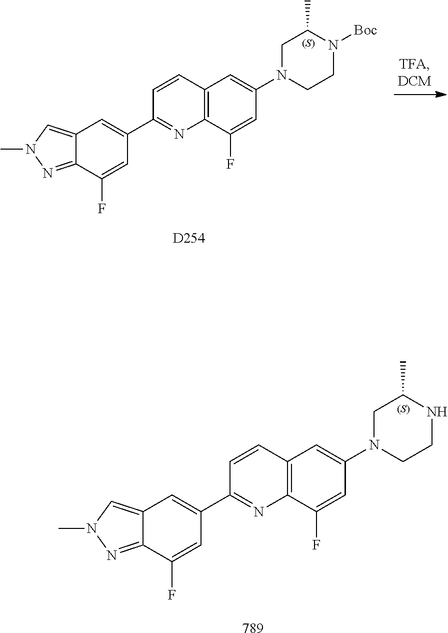

To a stirred solution of Intermediate D254 (280.0 mg, 0.567 mmol, 1.0 equiv) in DCM (5 mL) was added TFA (0.5 mL, 6.732 mmol) dropwise at room temperature. The resulting mixture was stirred for 1 h at room temperature and then concentrated under reduced pressure. The crude product was purified by Prep-HPLC following Condition 8, Gradient 3 as previously described to afford 8-fluoro-2-(7-fluoro-2-methylindazol-5-yl)-6-[(3S)-3-methylpiperazin-1-yl]quinoline (Compound 789, 65.9 mg). Absolute stereochemistry of the compound was arbitrarily assigned. LCMS: (ES, m/z): 394 [M+H]⁺. ¹H NMR: (400 MHz, DMSO-d₆) δ 8.62 (d, J=2.8 Hz, 1H), 8.45-8.40 (m, 1H), 8.24 (dd, J=8.9, 1.7 Hz, 1H), 8.16 (d, J=8.9 Hz, 1H), 7.99 (dd, J=13.6, 1.3 Hz, 1H), 7.52 (dd, J=14.5, 2.5 Hz, 1H), 7.03 (d, J=2.6 Hz, 1H), 4.24 (s, 3H), 3.74 (t, J=9.9 Hz, 2H), 3.00 (d, J=11.9 Hz, 1H), 2.82 (s, 1H), 2.88-2.77 (m, 1H), 2.71 (dd, J=11.6, 3.1 Hz, 1H), 2.39-2.29 (m, 1H), 1.07 (d, J=6.3 Hz, 3H).

### Example 298: Synthesis of Compound 790

### Synthesis of Intermediate D256

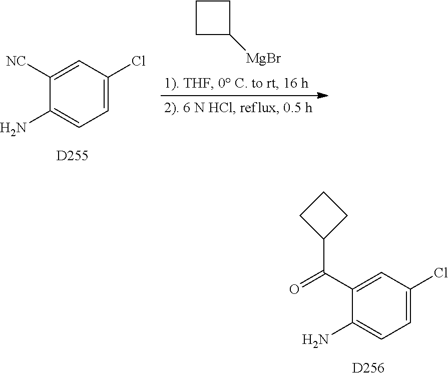

To a stirred solution of 2-amino-5-chlorobenzonitrile (D255, 3 g, 19.662 mmol, 1.0 equiv) in THF (10 mL) was added bromo(cyclobutyl)magnesium (60 mL, 60 mmol, 3.0 equiv, 1 mol/L in THF) dropwise at 0° C. The resulting mixture was stirred for 16 h at room temperature and then quenched with 6 M HCl (100 mL) at 0° C. The resulting mixture was stirred for an additional 0.5 h at reflux, basified to pH 7 with saturated aqueous NaHCO₃, and extracted with EA (3×50 mL). The combined organic layers were washed with brine (3×50 mL), dried over anhydrous Na₂SO₄, and the filtrate was concentrated under reduced pressure. The residue was purified by silica gel column chromatography and eluted with PE:EA (3:1) to afford 4-chloro-2-cyclobutanecarbonylaniline (D256, 800 mg). LCMS: (ES, m/z): 210 [M+H]⁺.

### Synthesis of Intermediate D257

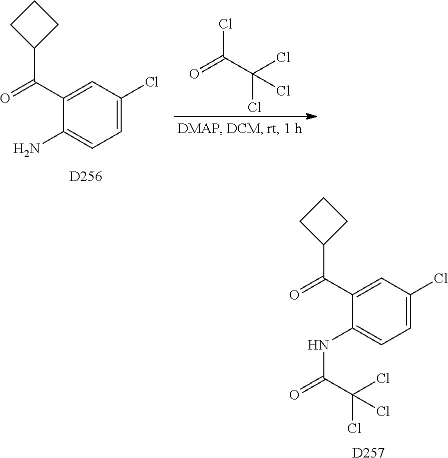

To a stirred solution of Intermediate D256 (900 mg, 4.292 mmol, 1.0 equiv) and DMAP (1.05 g, 8.584 mmol, 2.0 equiv) in DCM (20 mL) was added trichloroacetyl chloride (900 mg, 4.950 mmol, 1.2 equiv) dropwise at room temperature. The resulting mixture was stirred for 1 h at room temperature and then concentrated under reduced pressure. The residue was purified by silica gel column chromatography and eluted with PE:EA (10:1) to afford 2,2,2-trichloro-N-(4-chloro-2-cyclobutanecarbonylphenyl)acetamide (D257, 1.2 g). LCMS: (ES, m/z): 355 [M+H]⁺.

### Synthesis of Intermediate D258

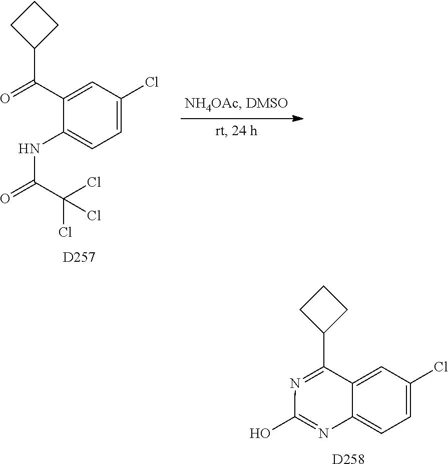

To a stirred solution of Intermediate D257 (1.2 g, 3.38 mmol, 1.0 equiv) in DMSO (20 mL) was added NH₄OAc (1.3 g, 16.9 mmol, 5.0 equiv) at room temperature. The resulting mixture was stirred for 24 h at room temperature, diluted with H₂O (20 mL), and then extracted with EA (3×20 mL). The combined organic layers were washed with brine (2×30 mL), dried over anhydrous Na₂SO₄, and the filtrate was concentrated under reduced pressure. The residue was purified by silica gel column chromatography and eluted with PE:EA (5:1) to afford 6-chloro-4-cyclobutylquinazolin-2-ol (D258, 700 mg). LCMS: (ES, m/z): 235 [M+H]⁺.

### Synthesis of Intermediate D259

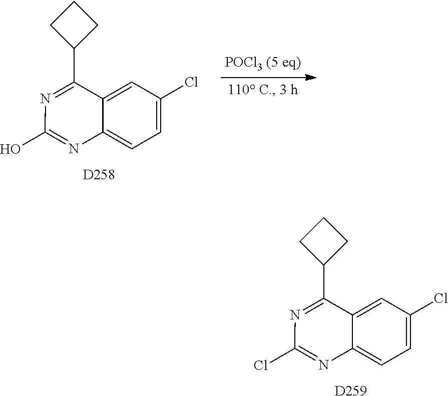

To a stirred solution of Intermediate D258 (700 mg, 2.983 mmol, 1.0 equiv) in dioxane (18 mL) was added phosphorus oxychloride (2.29 g, 14.915 mmol, 5.0 equiv) dropwise at room temperature. The resulting mixture was stirred for 3 h at 110° C., allowed to cool to room temperature, quenched by the addition of H₂O (50 mL) at room temperature, and then extracted with EA (3×30 mL). The combined organic layers were washed with brine (2×30 mL), dried over anhydrous Na₂SO₄, and the filtrate was concentrated under reduced pressure. The residue was purified by silica gel column chromatography and eluted with PE:EA (5:1) to afford 2,6-dichloro-4-cyclobutylquinazoline (D259, 550 mg). LCMS: (ES, m/z): 253 [M+H]⁺.

### Synthesis of Intermediate D260

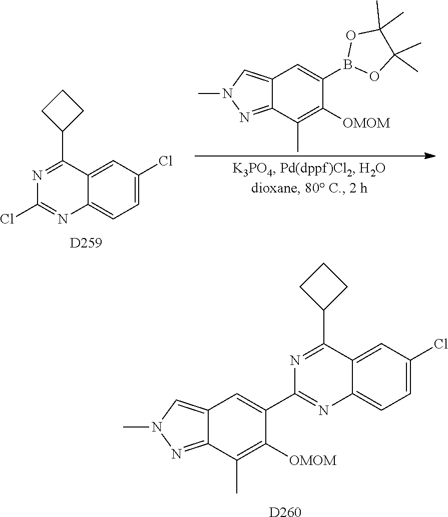

To a solution of Intermediate D259 (300 mg, 1.185 mmol, 1.0 equiv) and 6-(methoxymethoxy)-2,7-dimethyl-5-(4,4,5,5-tetramethyl-1,3,2-dioxaborolan-2-yl) indazole (590 mg, 1.778 mmol, 1.5 equiv) in dioxane (5 mL) and H₂O (1 mL) were added K₃PO₄ (754 mg, 3.555 mmol, 3.0 equiv) and Pd(dppf)Cl₂ (97 mg, 0.119 mmol, 0.1 equiv). After stirring for 2 h at 80° C. under a nitrogen atmosphere, the resulting mixture was concentrated under reduced pressure. The residue was purified by reversed-phase flash chromatography following Condition 10, Gradient 8 as previously described to afford 6-chloro-4-cyclobutyl-2-[6-(methoxymethoxy)-2,7-dimethylindazol-5-yl]quinazoline (D260, 250 mg). LCMS: (ES, m/z): 423 [M+H]⁺.

### Synthesis of Intermediate D261

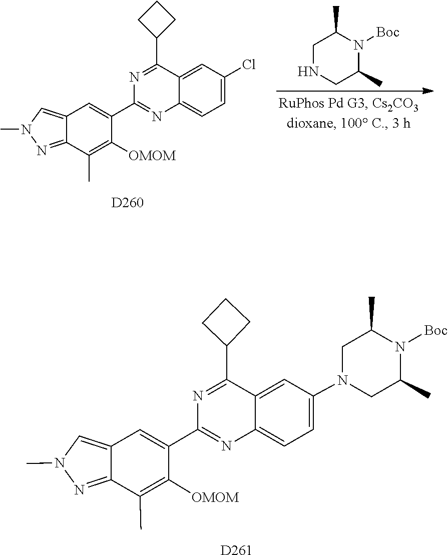

To a solution of Intermediate D260 (150 mg, 0.355 mmol, 1.0 equiv) and tert-butyl (2R,6S)-2,6-dimethylpiperazine-1-carboxylate (114 mg, 0.532 mmol, 1.5 equiv) in dioxane (3.5 mL) were added Cs₂CO₃ (346.6 mg, 1.065 mmol, 3.0 equiv), RuPhos (33.1 mg, 0.071 mmol, 0.2 equiv), and RuPhos Palladacycle Gen.3 (29.6 mg, 0.035 mmol, 0.1 equiv). After stirring for 3 h at 100° C. under a nitrogen atmosphere, the resulting mixture was concentrated under reduced pressure. The residue was purified by reversed-phase flash chromatography following Condition 10, Gradient 8 as previously described to afford tert-butyl (2R,6S)-4-{4-cyclobutyl-2-[6-(methoxymethoxy)-2,7-dimethylindazol-5-yl]quinazolin-6-yl}-2,6-dimethylpiperazine-1-carboxylate (D261, 174 mg). LCMS: (ES, m/z): 601 [M+H]⁺.

### Synthesis of Compound 790

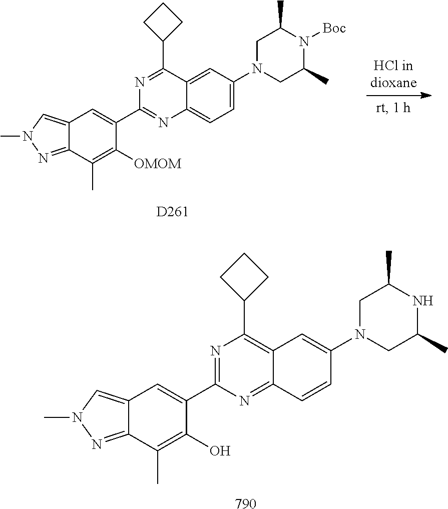

To a stirred solution of Intermediate D261 (170 mg, 0.283 mmol, 1.0 equiv) in DCM (2 mL) was added 4 M HCl (gas) in 1,4-dioxane (2 mL) dropwise at room temperature. The resulting mixture was stirred for 1 h at room temperature and then concentrated under reduced pressure. The residue was purified by reversed-phase flash chromatography following Condition 10, Gradient 10 as previously described to afford 5-{4-cyclobutyl-6-[(3R,5S)-3,5-dimethylpiperazin-1-yl]quinazolin-2-yl}-2,7-dimethylindazol-6-ol (Compound 790, 70 mg). Absolute stereochemistry of the compound was arbitrarily assigned. LCMS: (ES, m/z): 457 [M+H]⁺. ¹H NMR: (300 MHz, DMSO-d₆) δ 13.90 (s, 1H), 8.98 (s, 1H), 8.42 (s, 1H), 7.91-7.87 (m, 2H), 7.17 (s, 1H), 4.53 (p, J=8.5 Hz, 1H), 4.15 (s, 3H), 3.85-3.75 (m, 2H), 2.89 (s, 2H), 2.70-2.54 (m, 4H), 2.40 (s, 3H), 2.36-2.15 (m, 3H), 1.99 (s, 1H), 1.08 (d, J=6.2 Hz, 6H).

### Example 299: Synthesis of Compound 791

### Synthesis of Intermediate D262

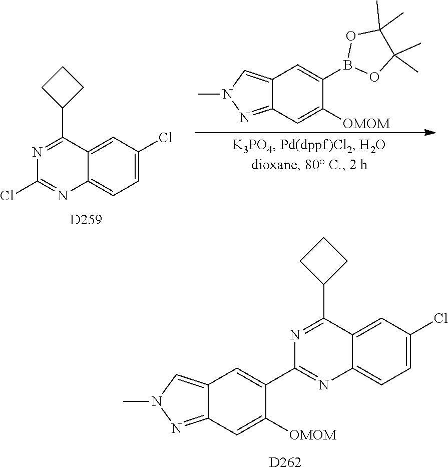

To a solution of Intermediate D259 (250 mg, 0.988 mmol, 1.0 equiv) and 6-(methoxymethoxy)-2-methyl-5-(4,4,5,5-tetramethyl-1,3,2-dioxaborolan-2-yl) indazole (471 mg, 1.482 mmol, 1.5 equiv) in dioxane (5 mL) and H₂O (1 mL) were added K₃PO₄ (628 mg, 2.964 mmol, 3.0 equiv) and Pd(dppf)Cl₂·CH₂Cl₂ (80 mg, 0.099 mmol, 0.1 equiv). After stirring for 2 h at 80° C. under a nitrogen atmosphere, the resulting mixture was concentrated under reduced pressure. The residue was purified by reversed-phase flash chromatography following Condition 10, Gradient 8 as previously described to afford 6-chloro-4-cyclobutyl-2-[6-(methoxymethoxy)-2-methylindazol-5-yl]quinazoline (D262, 200 mg). LCMS: (ES, m/z): 409 [M+H]⁺.

### Synthesis of Intermediate D263

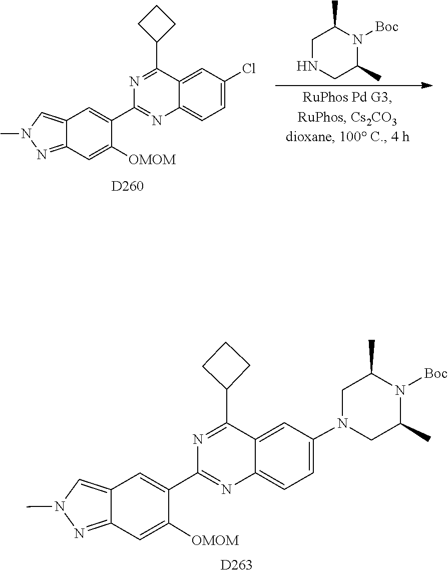

To a solution of Intermediate D262 (230 mg, 0.562 mmol, 1.0 equiv) and tert-butyl (2R,6S)-2,6-dimethylpiperazine-1-carboxylate (180 mg, 0.843 mmol, 1.5 equiv) in dioxane (3 mL) were added Cs₂CO₃ (549.8 mg, 1.686 mmol, 3.0 equiv), RuPhos (52.5 mg, 0.112 mmol, 0.2 equiv) and RuPhos Palladacycle Gen.3 (47 mg, 0.056 mmol, 0.1 equiv). After stirring for 4 h at 100° C. under a nitrogen atmosphere, the resulting mixture was concentrated under reduced pressure. The residue was purified by reversed-phase flash chromatography following Condition 10, Gradient 8 as previously described to afford tert-butyl (2R,6S)-4-{4-cyclobutyl-2-[6-(methoxymethoxy)-2-methylindazol-5-yl]quinazolin-6-yl}-2,6-dimethylpiperazine-1-carboxylate (D263, 288 mg). LCMS: (ES, m/z): 587 [M+H]⁺.

### Synthesis of Compound 791

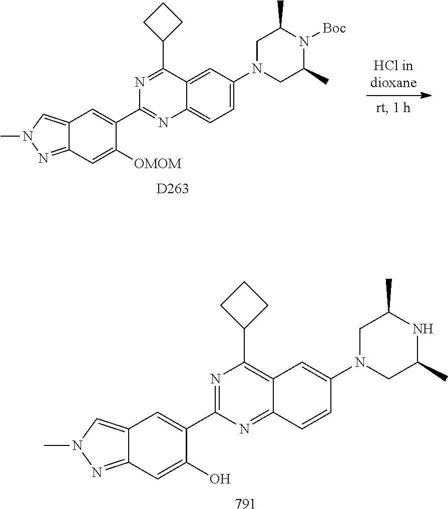

To a stirred solution of Intermediate D263 (180 mg, 0.307 mmol, 1.0 equiv) in DCM (2 mL) was added 4 M HCl (gas) in 1,4-dioxane (2 mL) dropwise at room temperature. The resulting mixture was stirred for 1 h at room temperature, basified to pH 8 with NH₃ (g) in MeOH, and then concentrated under reduced pressure. The residue was purified by reversed-phase flash chromatography following Condition 10, Gradient 10 as previously described to afford 5-{4-cyclobutyl-6-[(3R,5S)-3,5-dimethylpiperazin-1-yl]quinazolin-2-yl}-2-methylindazol-6-ol (Compound 791, 54 mg). Absolute stereochemistry of the compound was arbitrarily assigned. LCMS: (ES, m/z): 443 [M+H]⁺. ¹H NMR: (300 MHz, DMSO-d₆) δ 13.74 (s, 1H), 9.11 (s, 1H), 8.45 (s, 1H), 7.87 (s, 2H), 7.17 (s, 1H), 6.89 (s, 1H), 4.54 (p, J=8.6 Hz, 1H), 4.13 (s, 3H), 3.86-3.76 (m, 2H), 2.89 (ddd, J=9.6, 6.3, 2.8 Hz, 2H), 2.69-2.50 (m, 4H), 2.37-2.15 (m, 3H), 2.01-1.88 (m, 1H), 1.09 (d, J=6.2 Hz, 6H).

### Example 300: Synthesis of Compound 792

### Synthesis of Intermediate D264

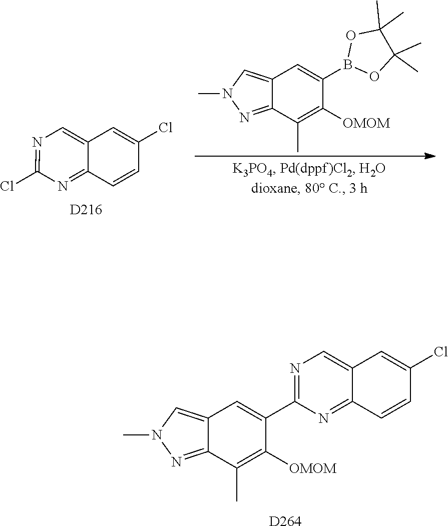

To a mixture of Intermediate D216 (210 mg, 1.055 mmol, 1.0 equiv) in 1,4-dioxane (2.5 mL)/H₂O (0.25 mL) were added 6-(methoxymethoxy)-2,7-dimethyl-5-(4,4,5,5-tetramethyl-1,3,2-dioxaborolan-2-yl) indazole (1331.9 mg, 2.004 mmol, 1.9 equiv, 50%), K₂CO₃ (437.5 mg, 3.165 mmol, 3 equiv) and Pd(dppf)Cl₂·CH₂Cl₂ (85.9 mg, 0.105 mmol, 0.1 equiv). The mixture was stirred for 3 h at 80° C. under nitrogen atmosphere, diluted with water (20 mL), and then extracted with DCM (2×20 mL). The combined organic layers were washed with water (1×40) and brine (2×40 mL), dried over anhydrous Na₂SO₄, and the filtrate was concentrated under reduced pressure. The residue was purified by silica gel column chromatography and eluted with PE/EA (1:4) to afford 6-chloro-2-[6-(methoxymethoxy)-2,7-dimethylindazol-5-yl]quinazoline (D264, 250 mg). LCMS: (ES, m/z):369 [M+H]⁺.

### Synthesis of Intermediate D265

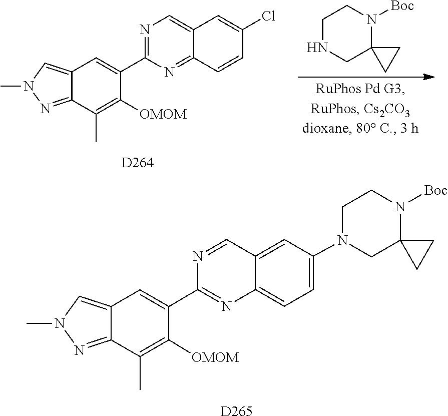

To a mixture of Intermediate D264 (250 mg, 0.678 mmol, 1.0 equiv) in 1,4-dioxane (3 mL) were added tert-butyl 4,7-diazaspiro[2.5]octane-4-carboxylate (172.68 mg, 0.814 mmol, 1.2 equiv), Cs₂CO₃ (103.21 mg, 1.356 mmol, 2.0 equiv), RuPhos (63.26 mg, 0.136 mmol, 0.20 equiv) and RuPhos Palladacycle Gen.3 (31.63 mg, 0.068 mmol, 0.10 equiv). The mixture was stirred for 3 h at 80° C. under nitrogen atmosphere, diluted with water (20 mL), and then extracted with DCM (2×20 mL). The combined organic layers were washed with brine (2×15 mL), dried over anhydrous Na₂SO₄, and the filtrate was concentrated under reduced pressure. The residue was purified by silica gel column chromatography and eluted with DCM/EA (1:2) to afford tert-butyl 7-{2-[6-(methoxymethoxy)-2,7-dimethylindazol-5-yl]quinazolin-6-yl}-4,7-diazaspiro[2.5]octane-4-carboxylate (D265, 190 mg). LCMS: (ES, m/z):545 [M+H]⁺.

### Synthesis of Compound 792

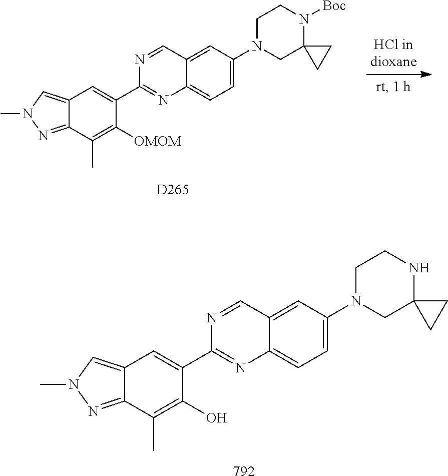

A mixture of Intermediate D265 (190 mg, 0.349 mmol, 1.0 equiv) in DCM (2 mL) was treated with HCl(gas) in 1,4-dioxane (1 mL) for 1 h at room temperature and subsequently concentrated under reduced pressure. The crude product was purified by Prep-HPLC following Condition 6, Gradient 8 as previously described to afford 5-(6-{4,7-diazaspiro[2.5]octan-7-yl}quinazolin-2-yl)-2,7-dimethylindazol-6-ol (Compound 792, 70 mg). LCMS: (ES, m/z):401 [M+H]⁺. ¹H NMR: (400 MHz, DMSO-d₆) δ 13.63 (s, 1H), 9.50 (s, 1H), 8.90 (s, 1H), 8.40 (s, 1H), 7.91 (d, J=1.8 Hz, 2H), 7.34 (d, J=2.1 Hz, 1H), 4.14 (s, 3H), 3.32-3.27 (m, 2H), 3.19 (s, 2H), 2.96 (t, J=5.1 Hz, 2H), 2.40 (s, 3H), 0.55 (dt, J=15.8, 1.8 Hz, 4H).

### Example 301: Synthesis of Compound 793

### Synthesis of Intermediate D267

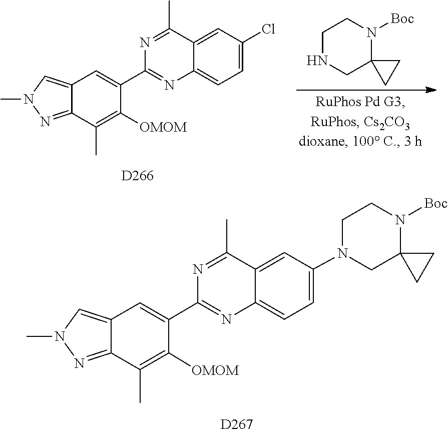

To a solution of 6-chloro-2-[6-(methoxymethoxy)-2,7-dimethylindazol-5-yl]-4-methylquinazoline (D266, 200 mg, 0.522 mmol, 1 equiv) and tert-butyl 4,7-diazaspiro[2.5]octane-4-carboxylate (166 mg, 0.783 mmol, 1.5 equiv) in dioxane (5 mL) were added Cs₂CO₃ (510 mg, 1.566 mmol, 3 equiv), RuPhos (48 mg, 0.104 mmol, 0.2 equiv) and RuPhos Palladacycle Gen.3 (43 mg, 0.052 mmol, 0.1 equiv). After stirring for 2 h at 100° C. under a nitrogen atmosphere, the resulting mixture was concentrated under reduced pressure. The residue was purified by silica gel column chromatography and eluted EA to afford tert-butyl 7-{2-[6-(methoxymethoxy)-2,7-dimethylindazol-5-yl]-4-methylquinazolin-6-yl}-4,7-diazaspiro[2.5]octane-4-carboxylate (D267, 250 mg). LCMS: (ES, m/z): 559 [M+H]⁺.

### Synthesis of Compound 793

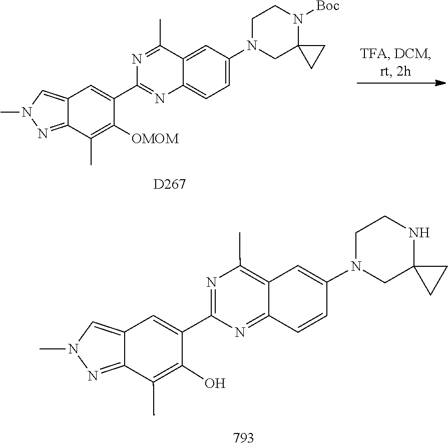

A solution of Intermediate D267 (250 mg, 0.447 mmol, 1 equiv) and trifluoroacetic acid (2 mL) in DCM (5 mL) was stirred for 2 h at room temperature and subsequently concentrated under vacuum. The residue was purified by reversed-phase flash chromatography following Condition 10, Gradient 1 as previously described to afford 5-(6-{4,7-diazaspiro[2.5]octan-7-yl}-4-methylquinazolin-2-yl)-2,7-dimethylindazol-6-ol (Compound 793, 60 mg). LCMS: (ES, m/z): 415 [M+H]⁺. ¹H NMR: (300 MHz, DMSO-d₆) δ 13.83 (s, 1H), 8.90 (s, 1H), 8.40 (s, 1H), 7.89 (d, J=9.3 Hz, 2H), 7.27 (s, 1H), 4.14 (s, 3H), 3.33 (s, 2H), 3.20 (s, 2H), 2.96 (s, 5H), 2.39 (s, 4H), 0.56 (d, J=17.4 Hz, 4H).

### Example 302: Synthesis of Compound 794

### Synthesis of Intermediate D268

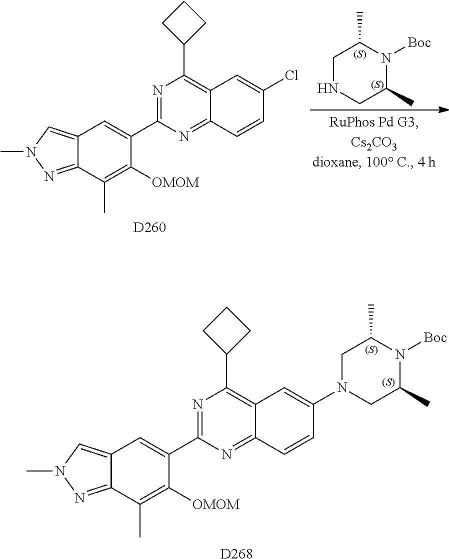

To a solution of 6-chloro-4-cyclobutyl-2-[6-(methoxymethoxy)-2,7-dimethylindazol-5-yl]quinazoline (D260, 100 mg, 0.236 mmol, 1.0 equiv) and tert-butyl (2S,6S)-2,6-dimethylpiperazine-1-carboxylate (80 mg, 0.373 mmol, 1.6 equiv) in dioxane (2 mL) were added Cs₂CO₃ (231.1 mg, 0.708 mmol, 3.0 equiv), RuPhos (22.0 mg, 0.047 mmol, 0.2 equiv) and RuPhos Palladacycle Gen.3 (19.7 mg, 0.024 mmol, 0.1 equiv). After stirring for 2 h at 100° C. under a nitrogen atmosphere, the resulting mixture was concentrated under reduced pressure. The residue was purified by reversed-phase flash chromatography following Condition 10, Gradient 8 as previously described to afford tert-butyl (2S,6S)-4-{4-cyclobutyl-2-[6-(methoxymethoxy)-2,7-dimethylindazol-5-yl]quinazolin-6-yl}-2,6-dimethylpiperazine-1-carboxylate (D268, 120 mg). LCMS: (ES, m/z): 601 [M+H]⁺.

### Synthesis of Compound 794

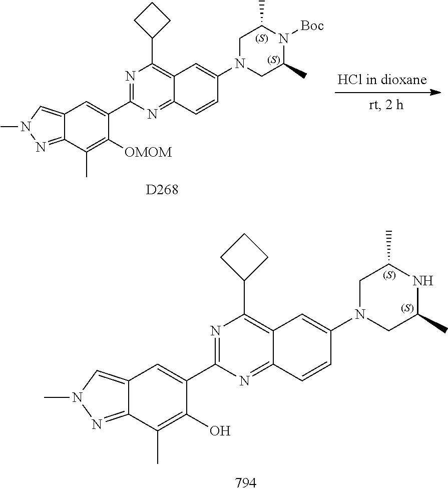

To a stirred solution of Intermediate D268 (120 mg, 0.2 mmol, 1.0 equiv) in DCM (2 mL) was added HCl (gas) in 1,4-dioxane (2 mL) dropwise at room temperature. The resulting mixture was stirred for 2 h at room temperature, basified to pH 8 with NH₃(g) in MeOH, and subsequently concentrated under reduced pressure. The residue was purified by reversed-phase flash chromatography following Condition 10, Gradient 8 as previously described to afford 5-{4-cyclobutyl-6-[(3S,5S)-3,5-dimethylpiperazin-1-yl]quinazolin-2-yl}-2,7-dimethylindazol-6-ol (Compound 794, 70 mg). Absolute stereochemistry of the compound was arbitrarily assigned. LCMS: (ES, m/z): 457 [M+H]⁺. ¹H NMR: (300 MHz, DMSO-d₆) δ 13.91 (s, 1H), 8.97 (s, 1H), 8.42 (s, 1H), 7.92-7.77 (m, 2H), 7.15 (d, J=2.5 Hz, 1H), 4.52 (p, J=8.5 Hz, 1H), 4.15 (s, 3H), 3.42-3.32 (m, 2H), 3.29-3.18 (m, 2H), 3.00 (dd, J=11.7, 6.2 Hz, 2H), 2.63-2.53 (m, 4H), 2.40 (s, 3H), 2.24 (dt, J=18.3, 9.0 Hz, 2H), 1.98 (t, J=7.5 Hz, 1H), 1.13 (d, J=6.4 Hz, 6H).

### Example 303: Synthesis of Compound 795

### Synthesis of Intermediate D270

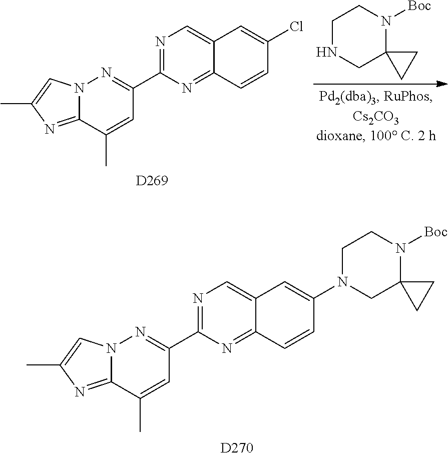

To a stirred mixture of 6-chloro-2-(2,8-dimethylimidazo[1,2-b]pyridazin-6-yl)quinazoline (D269, 200 mg, 0.647 mmol, 1.0 equiv) and tert-butyl 4,7-diazaspiro[2.5]octane-4-carboxylate (206 mg, 0.971 mmol, 1.5 equiv) in dioxane (4 mL) were added RuPhos (60 mg, 0.129 mmol, 0.20 equiv), Pd₂(dba)₃ (59 mg, 0.065 mmol, 0.10 equiv) and Cs₂CO₃ (422 mg, 1.294 mmol, 2.0 equiv) in portions. The resulting mixture was stirred for 2 h at 100° C. under nitrogen atmosphere and then concentrated under reduced pressure. The residue was purified by silica gel column chromatography and eluted with PE/EA (1:1) to afford ethyl tert-butyl 7-(2-(2,8-dimethylimidazo[1,2-b]pyridazin-6-yl)quinazolin-6-yl)-4,7-diazaspiro[2.5]octane-4-carboxylate (D270, 126 mg). LCMS: (ES, m/z): 486 [M+H]⁺.

### Synthesis of Compound 795

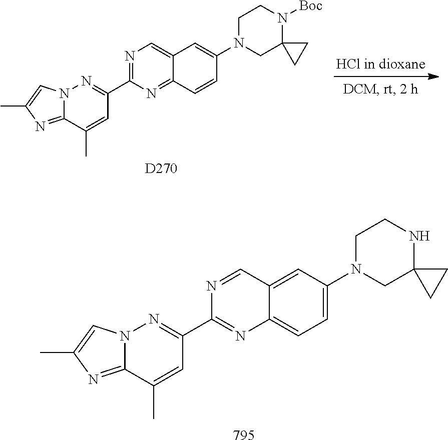 [SMILES: Cc1cn2nc(-c3ncc4cc(N5CCN(C(=O)OC(C)(C)C)C6(CC6)C5)ccc4n3)cc(C)c2n1 ; "D270" ; Cc1cn2nc(-c3ncc4cc(N5CCNC6(CC6)C5)ccc4n3)cc(C)c2n1 ; "795"]

To a stirred solution of Intermediate D270 (126 mg, 0.259 mmol, 1.0 equiv) in DCM (2.0 mL) was added HCl(gas) in 1,4-dioxane (0.4 mL) at room temperature. The resulting mixture was stirred for 1 h at room temperature and then concentrated under reduced pressure. The residue was purified by reversed-phase flash chromatography following Condition 9, Gradient 5 as previously described to afford 2-(2,8-dimethylimidazo[1,2-b]pyridazin-6-yl)-6-(4,7-diazaspiro[2.5]octan-7-yl)quinazoline (Compound 795, 41.6 mg). LCMS: (ES, m/z): 386 [M+H]⁺. ¹H NMR: (400 MHz, DMSO-d₆) δ 9.49 (s, 1H), 8.15 (d, J=1.0 Hz, 1H), 8.08 (d, J=1.3 Hz, 1H), 8.01-7.89 (m, 2H), 7.35 (d, J=2.6 Hz, 1H), 3.35 (s, 2H), 3.23 (s, 2H), 2.96 (t, J=5.1 Hz, 2H), 2.65 (d, J=1.1 Hz, 3H), 2.44 (s, 3H), 0.63-0.55 (m, 2H), 0.56-0.48 (m, 2H).

### Example 304: Synthesis of Compound 798

### Synthesis of Intermediate D269

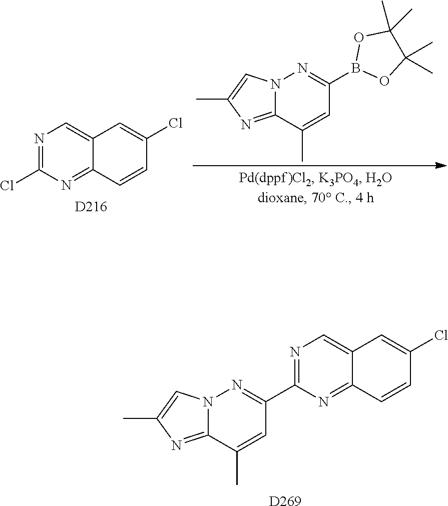

To a stirred mixture of Intermediate D216 (250 mg, 1.256 mmol, 1.0 equiv) and 2,8-dimethyl-6-(4,4,5,5-tetramethyl-1,3,2-dioxaborolan-2-yl)imidazo[1,2-b]pyridazine (412 mg, 1.508 mmol, 1.2 equiv) in H₂O (0.5 mL) and dioxane (5 mL) was added Pd(dppf)Cl₂ (92 mg, 0.126 mmol, 0.10 equiv) and K₃PO₄ (533 mg, 2.511 mmol, 2.0 equiv) at room temperature under nitrogen atmosphere. The resulting mixture was stirred for 4 h at 70° C., allowed to cool to room temperature, and then concentrated under reduced pressure. The residue was purified by silica gel column chromatography and eluted with PE/EA (1:1) to afford 6-chloro-2-{2,8-dimethylimidazo[1,2-b]pyridazin-6-yl}quinazoline (D269, 200 mg). LCMS: (ES, m/z): 310 [M+H]⁺.

### Synthesis of Intermediate D271

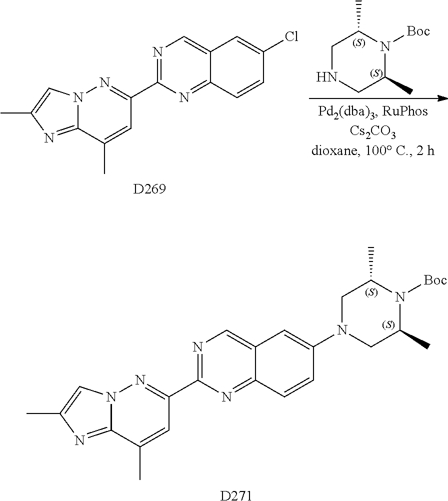

To a stirred mixture of Intermediate D269 (200 mg, 0.646 mmol, 1.0 equiv) and tert-butyl (2S,6S)-2,6-dimethylpiperazine-1-carboxylate (166 mg, 0.775 mmol, 1.2 equiv) in dioxane (4 mL) were added RuPhos (60 mg, 0.129 mmol, 0.20 equiv), Cs₂CO₃ (421 mg, 1.292 mmol, 2.0 equiv) and Pd₂(dba)₃ (59 mg, 0.064 mmol, 0.10 equiv) at room temperature. The resulting mixture was stirred for 2 h at 100° C. under nitrogen atmosphere and subsequently concentrated under reduced pressure. The residue was purified by silica gel column chromatography and eluted with PE/EA (1:1) to afford tert-butyl (2S,6S)-4-(2-{2,8-dimethylimidazo[1,2-b]pyridazin-6-yl}quinazolin-6-yl)-2,6-dimethylpiperazine-1-carboxylate (D271, 120 mg). LCMS: (ES, m/z): 488 [M+H]⁺.

### Synthesis of Compound 798

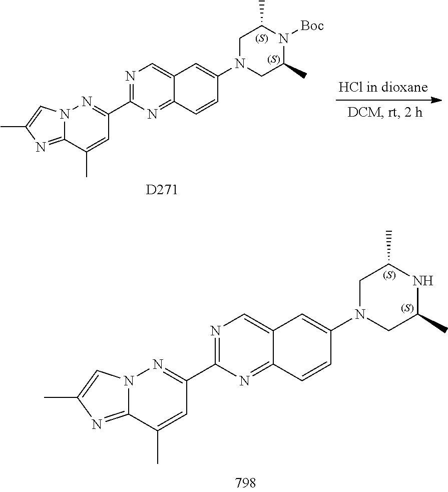

A mixture of Intermediate D271 (120 mg, 0.246 mmol, 1.0 equiv) and 4M HCl(gas) in 1,4-dioxane (1 mL) in DCM (2 mL) was stirred for 2 h at room temperature and then concentrated under reduced pressure. The crude product was purified by reversed-phase flash chromatography following Condition 9, Gradient 5 as previously described to afford 2-{2,8-dimethylimidazo[1,2-b]pyridazin-6-yl}-6-[(3S,5S)-3,5-dimethylpiperazin-1-yl]quinazoline (Compound 798, 43.5 mg). Absolute stereochemistry of the compound was arbitrarily assigned. LCMS: (ES, m/z): 388 [M+H]⁺. ¹H NMR: (400 MHz, DMSO-d₆) δ 9.49 (s, 1H), 8.15 (d, J=1.0 Hz, 1H), 8.08 (d, J=1.3 Hz, 1H), 8.00-7.89 (m, 2H), 7.37 (d, J=2.6 Hz, 1H), 3.41 (dd, J=11.9, 3.3 Hz, 2H), 3.3 (s, 1H), 3.24 (dd, J=6.4, 3.4 Hz, 2H), 3.06 (dd, J=11.9, 6.3 Hz, 2H), 2.65 (d, J=1.2 Hz, 3H), 2.44 (s, 3H), 1.13 (d, J=6.5 Hz, 6H).

### Example 305: Synthesis of Compound 705

### Synthesis of Intermediate D273

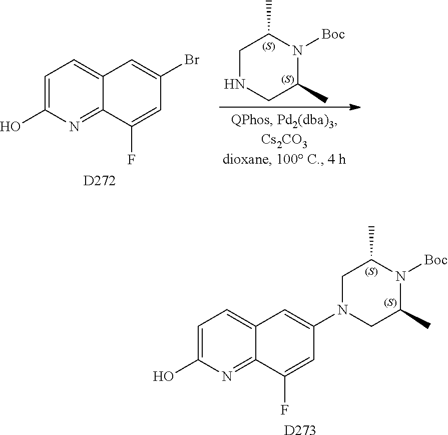

To a stirred mixture of 6-bromo-8-fluoroquinolin-2-ol (D272, 3 g, 12.394 mmol, 1.0 equiv) and tert-butyl (2S,6S)-2,6-dimethylpiperazine-1-carboxylate (3.9 g, 18.591 mmol, 1.5 equiv) in 1,4-dioxane (30 mL) were added t-BuONa (3.5 g, 37.182 mmol, 3.0 equiv), QPhos (1.7 g, 2.479 mmol, 0.2 equiv) and Pd₂(dba)₃ (1 g, 1.239 mmol, 0.1 equiv) at room temperature under nitrogen atmosphere. The resulting mixture was stirred for 4 h at 100° C., allowed to cool to room temperature, and then concentrated under reduced pressure. The residue was purified by silica gel column chromatography and eluted with PE/EA (1:1) to afford tert-butyl (2S,6S)-4-(8-fluoro-2-hydroxyquinolin-6-yl)-2,6-dimethylpiperazine-1-carboxylate (D273, 3.1 g). LCMS: (ES, m/z): 376 [M+H]⁺.

### Synthesis of Intermediate D274

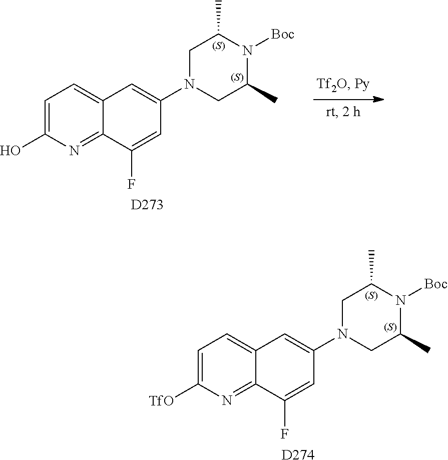

To a stirred mixture of Intermediate D273 (3.1 g, 8.257 mmol, 1.0 equiv) in pyridine (30 mL) was added Tf₂O (9.3 g, 33.028 mmol, 4 equiv) at room temperature under nitrogen atmosphere. The resulting mixture was stirred for 2 h at room temperature and then concentrated under reduced pressure. The residue was purified by silica gel column chromatography and eluted with PE/EA (5:1) to afford tert-butyl (2S,6S)-4-[8-fluoro-2-(trifluoromethanesulfonyloxy)quinolin-6-yl]-2,6-dimethylpiperazine-1-carboxylate (D274, 2.9 g). LCMS: (ES, m/z): 508 [M+H]⁺.

### Synthesis of Intermediate D275

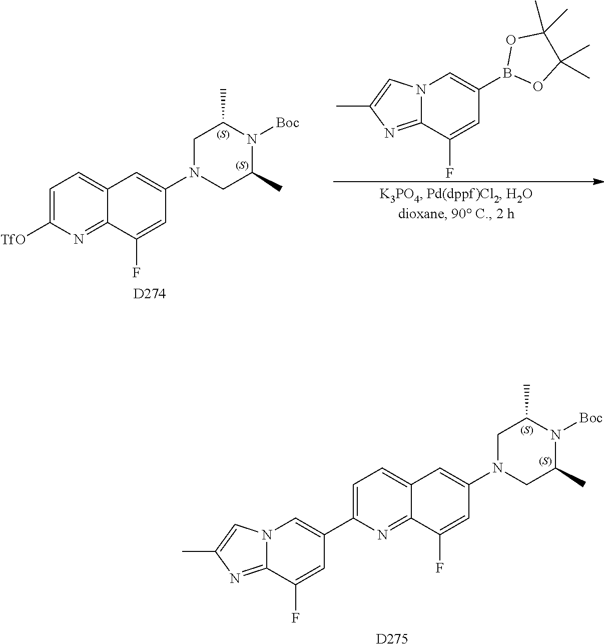

To a stirred mixture of Intermediate D274 (300 mg, 0.591 mmol, 1.0 equiv) and 8-fluoro-2-methyl-6-(4,4,5,5-tetramethyl-1,3,2-dioxaborolan-2-yl)imidazo[1,2-a]pyridine (244 mg, 0.886 mmol, 1.5 equiv) in 1,4-dioxane (10 mL) and H₂O (1 mL) were added K₃PO₄ (376 mg, 1.773 mmol, 3.0 equiv) and Pd(dppf)Cl₂ (43 mg, 0.059 mmol, 0.1 equiv) at room temperature under nitrogen atmosphere. The resulting mixture was stirred for 2 h at 90° C. under nitrogen atmosphere and subsequently concentrated under reduced pressure. The residue was purified by silica gel column chromatography and eluted with PE/EA (1:1) to afford tert-butyl (2S,6S)-4-(8-fluoro-2-{8-fluoro-2-methylimidazo[1,2-a]pyridin-6-yl}quinolin-6-yl)-2,6-dimethylpiperazine-1-carboxylate (D275, 270 mg). LCMS: (ES, m/z):508 [M+H]⁺.

### Synthesis of Compound 705

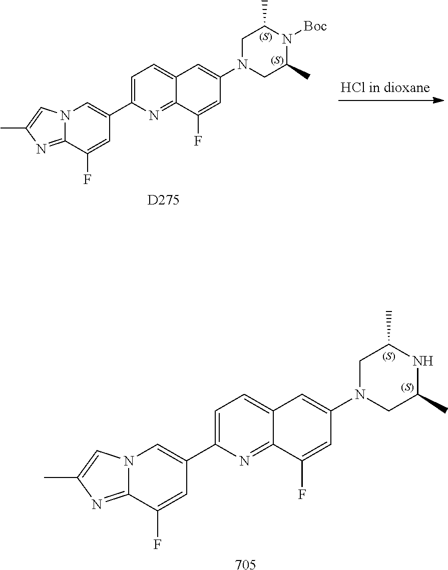

A mixture of Intermediate D275 (270 mg, 0.532 mmol, 1.0 equiv) and 4M HCl(gas) in 1,4-dioxane (3 mL) was stirred for 2 h at room temperature and then concentrated under reduced pressure. The residue was purified by reversed-phase flash chromatography following Condition 12, Gradient 1 as previously described to afford 6-((3S,5S)-3,5-dimethylpiperazin-1-yl)-8-fluoro-2-(8-fluoro-2-methylimidazo[1,2-a]pyridin-6-yl)quinoline 2,2,2-trifluoroacetate (Compound 705, 200 mg). Absolute stereochemistry of the compound was arbitrarily assigned. LCMS: (ES, m/z):522 [M+H]⁺. ¹H NMR: (300 MHz, DMSO-d₆) δ 9.35 (d, J=1.4 Hz, 1H), 8.96 (s, 1H), 8.33 (dd, J=8.9, 1.6 Hz, 1H), 8.15 (d, J=8.8 Hz, 1H), 8.09 (d, J=12.4 Hz, 1H), 8.04 (d, J=2.9 Hz, 1H), 7.65 (dd, J=13.9, 2.5 Hz, 1H), 7.18 (d, J=2.5 Hz, 1H), 3.82-3.57 (m, 4H), 3.35 (dd, J=13.4, 6.5 Hz, 2H), 2.47-2.40 (m, 3H), 1.36 (d, J=6.5 Hz, 6H).

### Example 306: Synthesis of Compound 799

### Synthesis of Intermediate D276

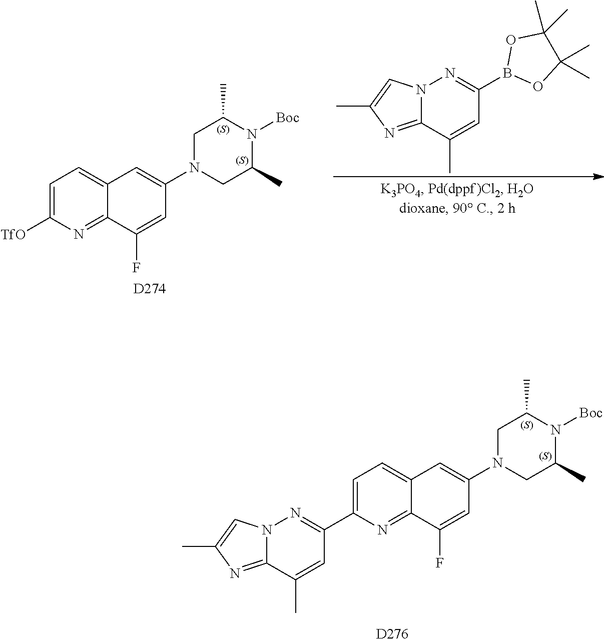

To a stirred mixture of tert-butyl (2S,6S)-4-[8-fluoro-2-(trifluoromethanesulfonyloxy)quinolin-6-yl]-2,6-dimethylpiperazine-1-carboxylate (D274, 300 mg, 0.591 mmol, 1.0 equiv) and 2,8-dimethyl-6-(4,4,5,5-tetramethyl-1,3,2-dioxaborolan-2-yl)imidazo[1,2-b]pyridazine (193 mg, 0.709 mmol, 1.2 equiv) in 1,4-dioxane (10 mL) and H₂O (1 mL) were added K₃PO₄ (376 mg, 1.773 mmol, 3.0 equiv) and Pd(dppf)Cl₂ (43 mg, 0.059 mmol, 0.1 equiv) at room temperature under nitrogen atmosphere. The resulting mixture was stirred for 2 h at 90° C. and subsequently concentrated under reduced pressure. The residue was purified by silica gel column chromatography and eluted with PE/EA (1:1) to afford tert-butyl (2S,6S)-4-(2-{2,8-dimethylimidazo[1,2-b]pyridazin-6-yl}-8-fluoroquinolin-6-yl)-2,6-dimethylpiperazine-1-carboxylate (D276, 260 mg). LCMS: (ES, m/z): 505 [M+H]⁺.

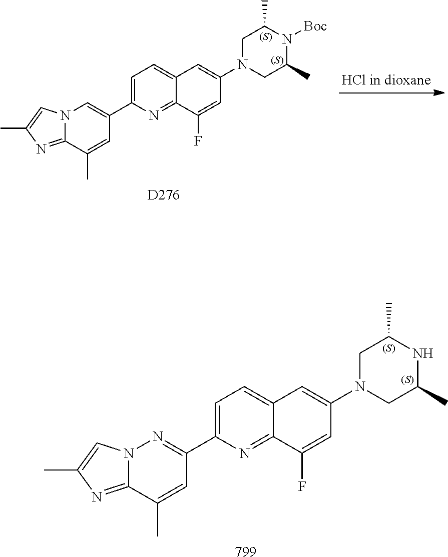

### Synthesis of Compound 799

A mixture of Intermediate D276 (260 mg, 0.515 mmol, 1.0 equiv) and 4M HCl(gas) in 1,4-dioxane (6 mL) was stirred for 2 h at room temperature and then concentrated under reduced pressure. The residue was purified by reversed-phase flash chromatography following Condition 10, Gradient 10 as previously described to afford 2-{2,8-dimethylimidazo[1,2-b]pyridazin-6-yl}-6-[(3S,5S)-3,5-dimethylpiperazin-1-yl]-8-fluoroquinoline (Compound 799, 80 mg). Absolute stereochemistry of the compound was arbitrarily assigned. LCMS: (ES, m/z): 405 [M+H]⁺. ¹H NMR: (300 MHz, DMSO-d₆) δ 8.37-8.23 (m, 2H), 8.08 (dd, J=8.4, 1.1 Hz, 2H), 7.56 (dd, J=14.5, 2.5 Hz, 1H), 7.06 (d, J=2.5 Hz, 1H), 3.45-3.35 (m, 2H), 3.30-3.18 (m, 2H), 3.03 (dd, J=11.9, 6.2 Hz, 2H), 2.66 (d, J=1.1 Hz, 3H), 2.43 (d, J=0.8 Hz, 3H), 1.13 (d, J=6.5 Hz, 6H).

### Example 307: Synthesis of Compound 760

### Synthesis of Intermediate D277

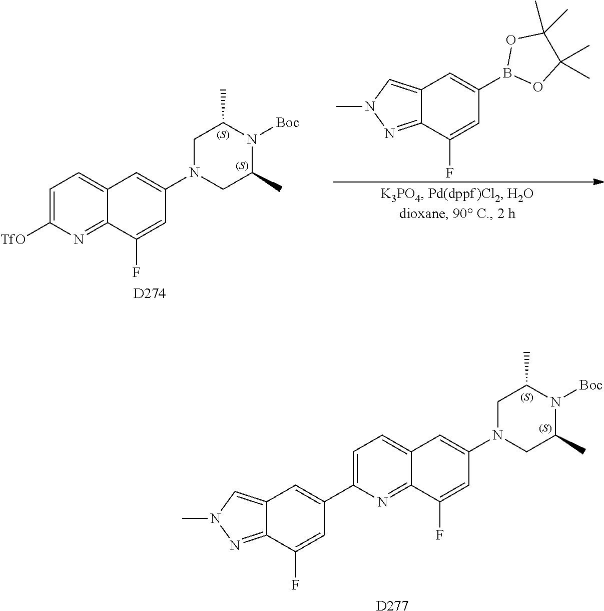

To a stirred mixture of tert-butyl (2S,6S)-4-[8-fluoro-2-(trifluoromethanesulfonyloxy)quinolin-6-yl]-2,6-dimethylpiperazine-1-carboxylate (D274, 300 mg, 0.591 mmol, 1.0 equiv) and 7-fluoro-2-methyl-5-(4,4,5,5-tetramethyl-1,3,2-dioxaborolan-2-yl) indazole (195 mg, 0.709 mmol, 1.2 equiv) in 1,4-dioxane (10 mL) and H₂O (1 mL) were added K₃PO₄ (376 mg, 1.773 mmol, 3.0 equiv) and Pd(dppf)Cl₂ (43 mg, 0.059 mmol, 0.10 equiv) at room temperature under nitrogen atmosphere. The resulting mixture was stirred for 2 h at 90° C. and then concentrated under reduced pressure. The residue was purified by silica gel column chromatography and eluted with PE/EA (1:1) to afford tert-butyl (2S,6S)-4-[8-fluoro-2-(7-fluoro-2-methylindazol-5-yl)quinolin-6-yl]-2,6-dimethylpiperazine-1-carboxylate (D277, 200 mg). LCMS: (ES, m/z): 508 [M+H]⁺.

### Synthesis of Compound 760

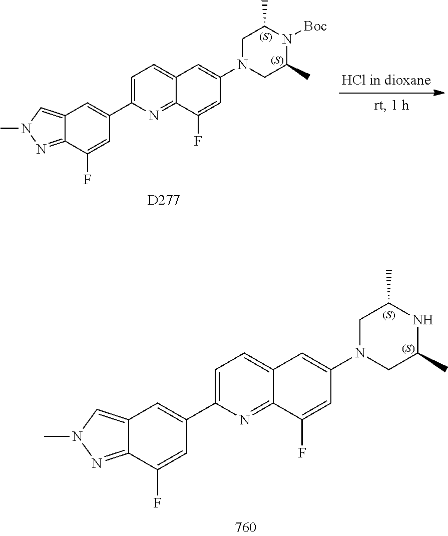

A mixture of Intermediate D277 (200 mg, 0.394 mmol, 1.0 equiv) and 4M HCl (gas) in 1,4-dioxane (4 mL) was stirred for 1 h at room temperature and subsequently concentrated under reduced pressure. The residue was purified by reversed-phase flash chromatography following Condition 10, Gradient 10 as previously described to afford 6-[(3S,5S)-3,5-dimethylpiperazin-1-yl]-8-fluoro-2-(7-fluoro-2-methylindazol-5-yl)quinoline (Compound 760, 80 mg). Absolute stereochemistry of the compound was arbitrarily assigned. LCMS: (ES, m/z): 408 [M+H]⁺. ¹H NMR: (300 MHz, DMSO-d₆) δ 8.62 (d, J=2.8 Hz, 1H), 8.43 (s, 1H), 8.23 (dd, J=8.9, 1.6 Hz, 1H), 8.16 (d, J=8.8 Hz, 1H), 7.99 (dd, J=13.7, 1.3 Hz, 1H), 7.49 (dd, J=14.5, 2.5 Hz, 1H), 7.02 (d, J=2.4 Hz, 1H), 4.24 (s, 3H), 3.39-3.18 (m, 4H), 2.97 (dd, J=11.6, 6.1 Hz, 2H), 1.13 (d, J=6.4 Hz, 6H).

### Example 308: Synthesis of Compound 800

### Synthesis of Intermediate D278

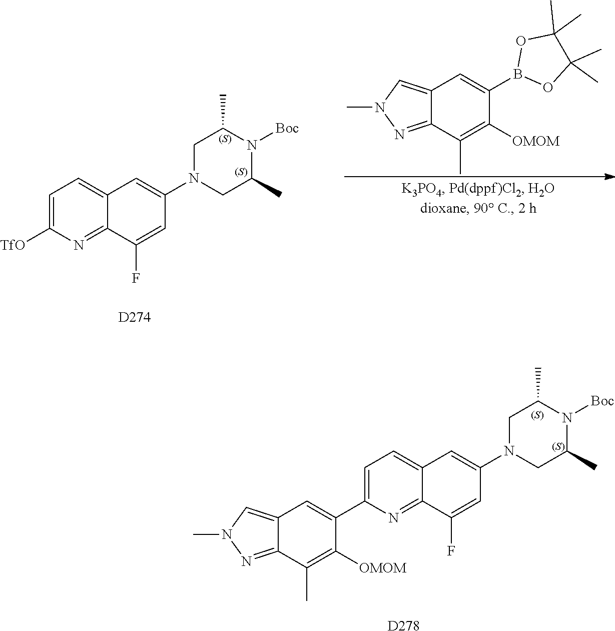

To a stirred solution of tert-butyl (2S,6S)-4-[8-fluoro-2-(trifluoromethanesulfonyloxy)quinolin-6-yl]-2,6-dimethylpiperazine-1-carboxylate (D274, 400 mg, 0.788 mmol, 1.0 equiv), K₃PO₄ (502 mg, 2.364 mmol, 3.0 equiv) and 6-(methoxymethoxy)-2,7-dimethyl-5-(4,4,5,5-tetramethyl-1,3,2-dioxaborolan-2-yl) indazole (393 mg, 1.182 mmol, 1.5 equiv) in dioxane (5 mL) were added H₂O (0.5 mL) and Pd(dppf)Cl₂ (60 mg, 0.079 mmol, 0.1 equiv) at room temperature under nitrogen atmosphere. The resulting mixture was stirred for 2 h at 90° C. under nitrogen atmosphere and then concentrated under reduced pressure. The residue was purified by silica gel column chromatography and eluted with PE/EA (1:4) to afford tert-butyl (2S,6S)-4-{8-fluoro-2-[6-(methoxymethoxy)-2,7-dimethylindazol-5-yl]quinolin-6-yl}-2,6-dimethylpiperazine-1-carboxylate (D278, 300 mg). LCMS: (ES, m/z): 564 [M+H]⁺.

### Synthesis of Compound 800

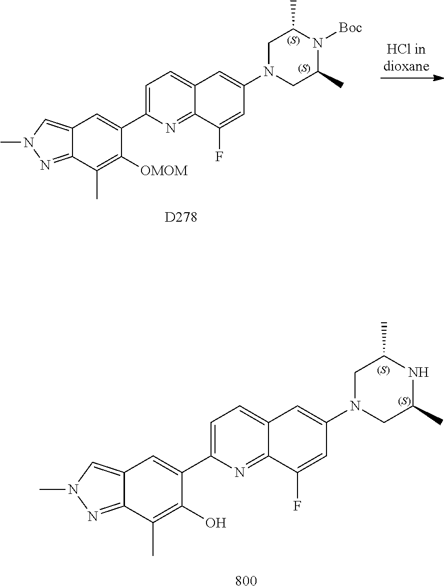

A solution of Intermediate D278 (290 mg, 0.514 mmol, 1.0 equiv) and 4 M HCl(gas) in 1,4-dioxane (4 mL) in DCM (6 mL) was stirred for 1 h at room temperature and subsequently concentrated under vacuum. The residue was purified by reversed-phase flash chromatography following Condition 4, Gradient 5 as previously described to afford 5-(6-((3S,5S)-3,5-dimethylpiperazin-1-yl)-8-fluoroquinolin-2-yl)-2,7-dimethyl-2H-indazol-6-ol 2,2,2-trifluoroacetate (Compound 800, 120 mg). Absolute stereochemistry of the compound was arbitrarily assigned. LCMS: (ES, m/z): 420 [M+H]⁺. ¹H NMR: (300 MHz, DMSO-d₆) δ 14.43 (s, 1H), 8.86 (s, 2H), 8.52 (s, 1H), 8.40-8.31 (m, 3H), 7.69 (dd, J=13.9, 2.5 Hz, 1H), 7.22 (d, J=2.4 Hz, 1H), 4.15 (s, 3H), 3.76 (s, 2H), 3.56 (s, 2H), 3.36 (dd, J=13.3, 6.5 Hz, 2H), 2.39 (s, 3H), 1.36 (d, J=6.6 Hz, 6H).

### Example 309: Synthesis of Compound 770

### Synthesis of Intermediate D279

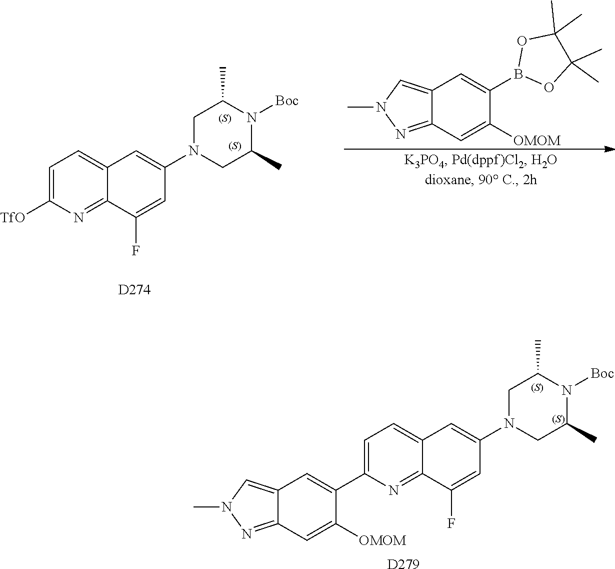

To a stirred solution of tert-butyl (2S,6S)-4-[8-fluoro-2-(trifluoromethanesulfonyloxy)quinolin-6-yl]-2,6-dimethylpiperazine-1-carboxylate (D274, 400 mg, 0.788 mmol, 1 equiv), K₃PO₄ (501 mg, 2.364 mmol, 3 equiv) and 6-(methoxymethoxy)-2-methyl-5-(4,4,5,5-tetramethyl-1,3,2-dioxaborolan-2-yl) indazole (37 mg, 1.182 mmol, 1.5 equiv) in dioxane (5 mL) were added H₂O (0.5 mL) and Pd(dppf)Cl₂ (57 mg, 0.079 mmol, 0.1 equiv) at room temperature under nitrogen atmosphere. The resulting mixture was stirred for 2 h at 90° C. under nitrogen atmosphere and then concentrated under reduced pressure. The residue was purified by silica gel column chromatography and eluted with PE/EA (1:4) to afford tert-butyl (2S,6S)-4-{8-fluoro-2-[6-(methoxymethoxy)-2-methylindazol-5-yl]quinolin-6-yl}-2,6-dimethylpiperazine-1-carboxylate (D279, 260 mg). LCMS: (ES, m/z): 550 [M+H]⁺.

### Synthesis of Compound 770

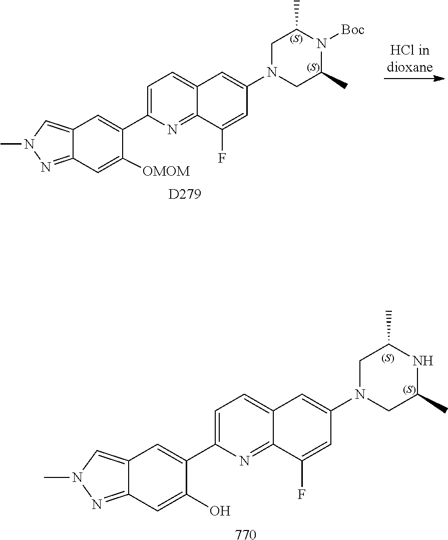

A solution of Intermediate D279 (260 mg, 0.473 mmol, 1 equiv) in 4 M HCl (gas) in 1,4-dioxane (5 mL) was stirred for 2 h at room temperature and subsequently concentrated under vacuum. The residue was purified by reversed-phase flash chromatography following Condition 10, Gradient 8 as previously described to afford 5-{6-[(3S,5S)-3,5-dimethylpiperazin-1-yl]-8-fluoroquinolin-2-yl}-2-methylindazol-6-ol (Compound 770, 40 mg). Absolute stereochemistry of the compound was arbitrarily assigned. LCMS: (ES, m/z): 405 [M+H]⁺. ¹H NMR: (300 MHz, DMSO-d₆) δ 14.27 (s, 1H), 8.63 (s, 1H), 8.42-8.28 (m, 3H), 7.57 (d, J=12.6 Hz, 1H), 7.08 (d, J=2.5 Hz, 1H), 6.88 (s, 1H), 4.12 (s, 3H), 3.37 (s, 2H), 3.25-3.17 (m, 2H), 2.99 (dd, J=11.7, 6.2 Hz, 2H), 2.13 (s, 1H), 1.12 (d, J=6.4 Hz, 6H).

### Example 310: Synthesis of Compound 801

### Synthesis of Intermediate D281

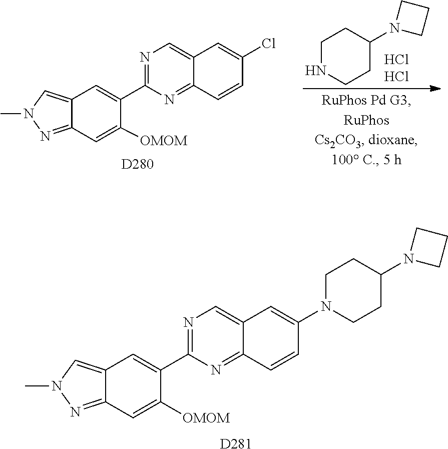

To a stirred mixture of 6-chloro-2-[6-(methoxymethoxy)-2-methylindazol-5-yl]quinazoline (D280, 300.0 mg, 0.846 mmol, 1 equiv), Cs₂CO₃ (268.6 mg, 0.825 mmol, 2.2 equiv) and 4-(azetidin-1-yl)piperidine (177.8 mg, 1.269 mmol, 1.5 equiv) in dioxane (8 mL) were added RuPhos (78.9 mg, 0.169 mmol, 0.2 equiv) and RuPhos Pd G3 (57.9 mg, 0.1 equiv) at room temperature under nitrogen atmosphere. The resulting mixture was stirred for 5 h at 100° C. and then concentrated under reduced pressure. The residue was purified by silica gel column chromatography and eluted with PE/EA (1:1) to afford 6-[4-(azetidin-1-yl)piperidin-1-yl]-2-[6-(methoxymethoxy)-2-methylindazol-5-yl]quinazoline (D281, 180 mg). LCMS: (ES, m/z): 459 [M+H]⁺.

### Synthesis of Compound 801

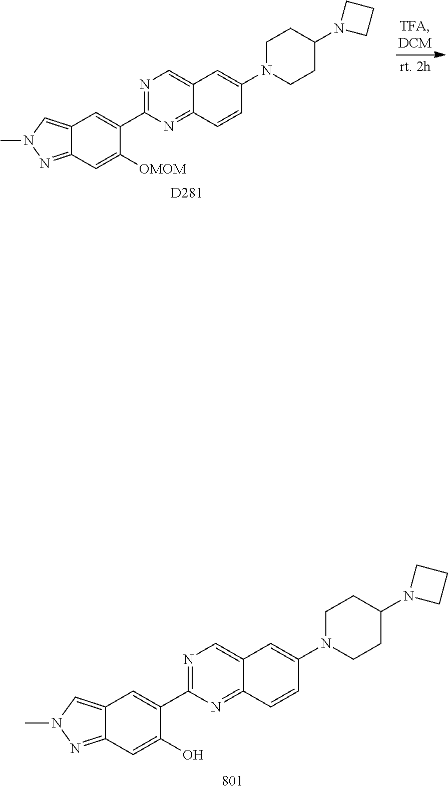

To a stirred solution of Intermediate D281 (180.0 mg, 0.393 mmol, 1 equiv) in DCM (2 mL) was added TFA (1 mL) dropwise at room temperature, stirred for 2 h, and then concentrated under reduced pressure. The crude product was purified by Prep-HPLC following Condition 6, Gradient 10 as previously described to afford 5-{6-[4-(azetidin-1-yl)piperidin-1-yl]quinazolin-2-yl}-2-methylindazol-6-ol (Compound 801, 4.9 mg). LCMS: (ES, m/z): 415 [M+H]⁺. ¹H NMR: (400 MHz, DMSO-d₆) δ 9.51 (s, 1H), 9.04 (s, 1H), 8.43 (s, 1H), 7.92 (d, J=2.6 Hz, 2H), 7.40 (s, 1H), 6.89 (s, 1H), 4.12 (s, 3H), 3.77 (d, J=13.0 Hz, 3H), 3.13 (d, J=6.8 Hz, 4H), 3.08-2.98 (m, 2H), 2.23-2.21 (m, 1H), 1.95-1.94 (m, 2H), 1.76 (d, J=12.6 Hz, 2H), 1.30-1.25 (m, 3H).

### Example 311: Synthesis of Compound 802

### Synthesis of Intermediate D283

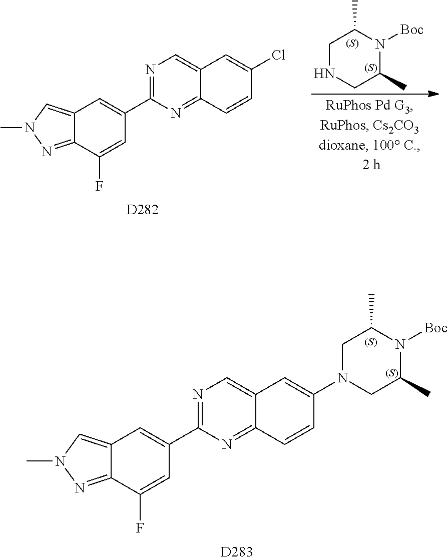

To a stirred solution of 6-chloro-2-(7-fluoro-2-methylindazol-5-yl)quinazoline (D282, 200 mg, 0.640 mmol, 1 equiv) and tert-butyl (2S,6S)-2,6-dimethylpiperazine-1-carboxylate (165 mg, 0.768 mmol, 1.2 equiv) in dioxane (3 mL) were added RuPhos (60 mg, 0.128 mmol, 0.2 equiv), Cs₂CO₃ (625 mg, 1.920 mmol, 3 equiv) and RuPhos Palladacycle Gen.3 (53 mg, 0.064 mmol, 0.1 equiv) at room temperature. The resulting mixture was stirred for 2 h at 100° C. under nitrogen atmosphere and then concentrated under reduced pressure. The residue was purified by silica gel column chromatography and eluted with PE/EA (1:1) to afford tert-butyl (2S,6S)-4-[2-(7-fluoro-2-methylindazol-5-yl)quinazolin-6-yl]-2,6-dimethylpiperazine-1-carboxylate (D283, 150 mg). LCMS: (ES, m/z): 491 [M+H]⁺.

### Synthesis of Compound 802

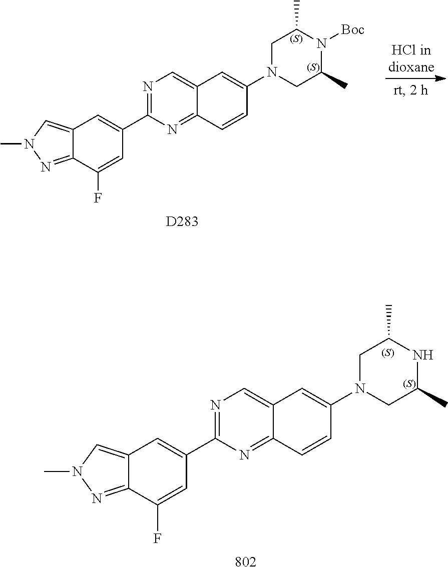

To a stirred mixture of Intermediate D283 (150 mg, 0.306 mmol, 1 equiv) in 4 M HCl (gas) in 1,4-dioxane (3 mL) was stirred for 2 h at room temperature and subsequently concentrated under reduced pressure. The residue was purified by reversed-phase flash chromatography following Condition 10, Gradient 10 as previously described to afford 6-[(3S,5S)-3,5-dimethylpiperazin-1-yl]-2-(7-fluoro-2-methylindazol-5-yl)quinazoline (Compound 802, 80 mg). Absolute stereochemistry of the compound was arbitrarily assigned. LCMS: (ES, m/z): 391 [M+H]⁺. ¹H NMR: (300 MHz, DMSO-d₆) δ 9.42 (s, 1H), 8.75 (d, J=1.2 Hz, 1H), 8.64 (d, J=2.8 Hz, 1H), 8.09 (dd, J=13.6, 1.2 Hz, 1H), 7.86 (d, J=3.3 Hz, 2H), 7.30 (d, J=2.3 Hz, 1H), 4.24 (s, 3H), 3.39-3.18 (m, 3H), 2.99 (dd, J=11.6, 6.1 Hz, 2H), 2.10 (s, 1H), 1.13 (d, J=6.4 Hz, 6H).

### Example 312: Synthesis of Compound 803

### Synthesis of Intermediate D285

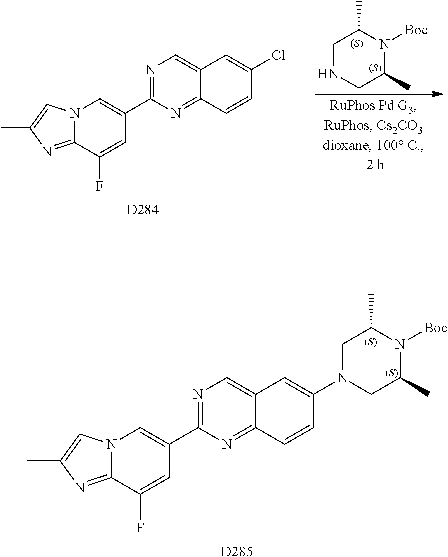

To a stirred solution of 6-chloro-2-{8-fluoro-2-methylimidazo[1,2-a]pyridin-6-yl}quinazoline (D284, 150 mg, 0.480 mmol, 1.0 equiv) and tert-butyl (2S,6S)-2,6-dimethylpiperazine-1-carboxylate (125 mg, 0.576 mmol, 1.2 equiv) in dioxane (5 mL) were added Cs₂CO₃ (470 mg, 1.440 mmol, 3 equiv), RuPhos (45 mg, 0.096 mmol, 0.2 equiv) and RuPhos Palladacycle Gen.3 (22 mg, 0.048 mmol, 0.1 equiv) at room temperature. The resulting mixture was stirred for 2 h at 100° C. under nitrogen atmosphere and then concentrated under reduced pressure. The residue was purified by silica gel column chromatography and eluted with PE/EA (1:1) to afford tert-butyl (2S,6S)-4-(2-{8-fluoro-2-methylimidazo[1,2-a]pyridin-6-yl}quinazolin-6-yl)-2,6-dimethylpiperazine-1-carboxylate (D285, 120 mg). LCMS: (ES, m/z): 491 [M+H]⁺.

### Synthesis of Compound 803

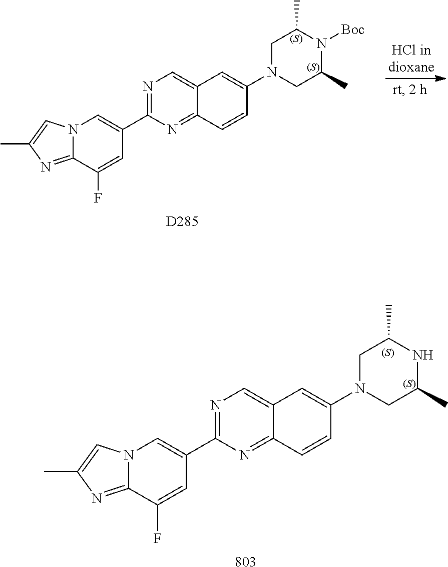

A solution of Intermediate D285 (120 mg, 0.245 mmol, 1.0 equiv) in 4M HCl(gas) in 1,4-dioxane (3 mL) was stirred for 2 h at room temperature and subsequently concentrated under reduced pressure. The residue was purified by reversed-phase flash chromatography following Condition 10, Gradient 10 as previously described to afford 6-[(3S,5S)-3,5-dimethylpiperazin-1-yl]-2-{8-fluoro-2-methylimidazo[1,2-a]pyridin-6-yl}quinazoline (Compound 803, 60 mg). Absolute stereochemistry of the compound was arbitrarily assigned. LCMS: (ES, m/z): 391 [M+H]⁺. ¹H NMR: (300 MHz, DMSO-d₆) δ 9.44 (s, 2H), 8.02 (d, J=3.1 Hz, 1H), 7.96 (d, J=12.6 Hz, 1H), 7.88 (s, 2H), 7.33 (s, 1H), 3.41-3.39 (m, 2H), 3.24 (dd, J=8.6, 5.2 Hz, 3H), 3.00 (dd, J=11.7, 6.2 Hz, 2H), 2.39 (s, 3H), 1.13 (d, J=6.4 Hz, 6H).

### Example 313: Synthesis of Compound 804

### Synthesis of Intermediate D286

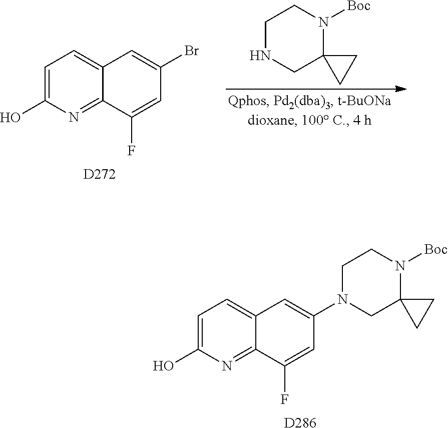

To a stirred solution of 6-bromo-8-fluoroquinolin-2-ol (D272, 300 mg, 1.239 mmol, 1.0 equiv) and tert-butyl 4,7-diazaspiro[2.5]octane-4-carboxylate (395 mg, 1.859 mmol, 1.5 equiv) in dioxane (6 mL) were added t-BuONa (240 mg, 2.478 mmol, 2.0 equiv), 1,2,3,4,5-pentaphenyl-1-(di-tert-butylphosphino)ferrocene (177 mg, 0.248 mmol, 0.20 equiv) and Pd₂(dba)₃ (114 mg, 0.124 mmol, 0.10 equiv) at room temperature under nitrogen atmosphere. The resulting mixture was stirred for 4 h at 100° C. under nitrogen atmosphere, allowed to cool to room temperature, and then concentrated under reduced pressure. The residue was purified by silica gel column chromatography and eluted with PE/EA (5:1) to afford tert-butyl 7-(8-fluoro-2-hydroxyquinolin-6-yl)-4,7-diazaspiro[2.5]octane-4-carboxylate (D286, 300 mg). LCMS: (ES, m/z): 374 [M+H]⁺.

### Synthesis of Intermediate D287

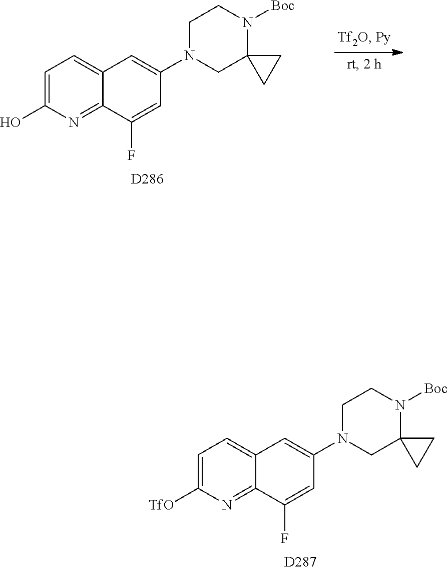

To a stirred solution of Intermediate D286 (300 mg, 0.803 mmol, 1.0 equiv) in pyridine (10 mL) was added Tf₂O (454 mg, 1.606 mmol, 2.0 equiv) dropwise at 0° C. The resulting mixture was stirred for 2 h at room temperature, quenched with water (20 mL) at room temperature, and subsequently extracted with EA (3×10 mL). The combined organic layers were washed with water (3×5 mL), dried over anhydrous Na₂SO₄, and the filtrate was concentrated under reduced pressure. The residue was purified by silica gel column chromatography and eluted with PE/EA (1:1) to afford tert-butyl 7-[8-fluoro-2-(trifluoromethanesulfonyloxy)quinolin-6-yl]-4,7-diazaspiro[2.5]octane-4-carboxylate (D287, 340 mg). LCMS: (ES, m/z): 506 [M+H]⁺.

### Synthesis of Intermediate D288

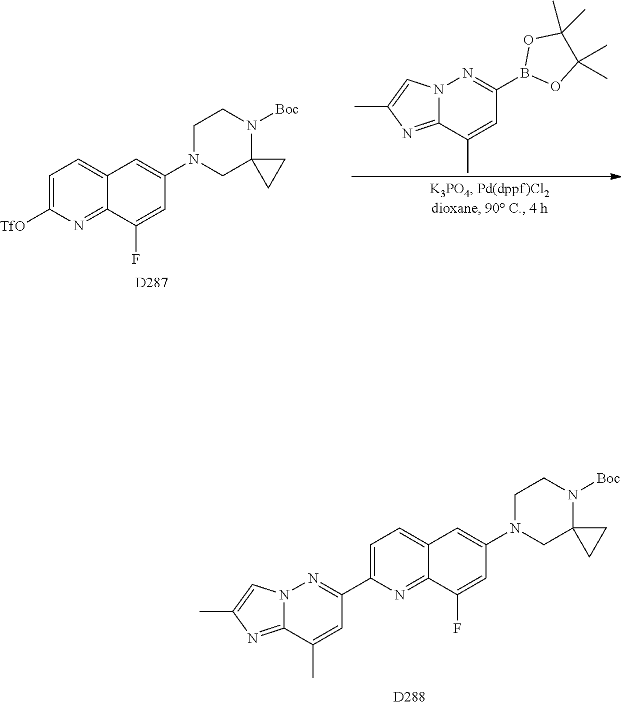

To a stirred solution of Intermediate D287 (340 mg, 0.673 mmol, 1.0 equiv) and 2,8-dimethyl-6-(4,4,5,5-tetramethyl-1,3,2-dioxaborolan-2-yl)imidazo[1,2-b]pyridazine (276 mg, 1.010 mmol, 1.5 equiv) in dioxane (6 mL) and H₂O (0.5 mL) were added K₃PO₄ (286 mg, 1.346 mmol, 2.0 equiv) and Pd(dppf)Cl₂ (55 mg, 0.067 mmol, 0.10 equiv) at room temperature under nitrogen atmosphere. The resulting mixture was stirred for 4 h at 90° C. under nitrogen atmosphere, allowed to cool to room temperature, and then concentrated under reduced pressure. The residue was purified by silica gel column chromatography and eluted with PE/EA (3:1) to afford tert-butyl 7-(2-{2,8-dimethylimidazo[1,2-b]pyridazin-6-yl}-8-fluoroquinolin-6-yl)-4,7-diazaspiro[2.5]octane-4-carboxylate (D288, 240 mg). LCMS: (ES, m/z): 503 [M+H]⁺.

### Synthesis of Compound 804

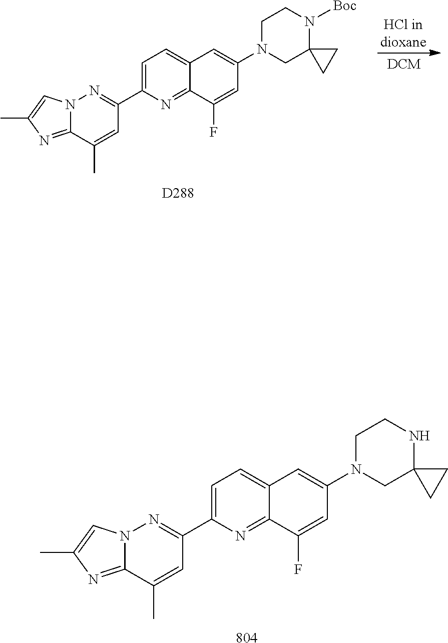 [SMILES: Cc1cn2nc(-c3ccc4cc(N5CCN(C(=O)OC(C)(C)C)C6(CC6)C5)cc(F)c4n3)cc(C)c2n1 ; "D288" ; Cc1cn2nc(-c3ccc4cc(N5CCNC6(CC6)C5)cc(F)c4n3)cc(C)c2n1 ; "804"]

A solution of Intermediate D288 (240 mg, 0.478 mmol, 1. equiv) and HCl(gas) in 1,4-dioxane (1 mL) in DCM (5 mL) was stirred for 2 h at room temperature and subsequently concentrated under reduced pressure. The residue was purified by reversed-phase flash chromatography following Condition 10, Gradient 14 as previously described to afford 6-{4,7-diazaspiro[2.5]octan-7-yl}-2-{2,8-dimethylimidazo[1,2-b]pyridazin-6-yl}-8-fluoroquinoline (Compound 804, 115 mg). LCMS: (ES, m/z): 403 [M+H]⁺. ¹H NMR: (300 MHz, Chloroform-d) δ 8.44 (d, J=8.7 Hz, 1H), 8.22 (d, J=1.3 Hz, 1H), 8.15-8.06 (m, 1H), 7.79 (s, 1H), 7.21 (dd, J=13.4, 2.5 Hz, 1H), 6.84 (s, 1H), 3.47-3.38 (m, 2H), 3.31-3.20 (m, 4H), 2.77 (d, J=1.1 Hz, 3H), 2.57 (s, 3H), 0.91 (s, 2H), 0.75 (s, 2H).

### Example 314: Synthesis of Compound 805

### Synthesis of Intermediate D289

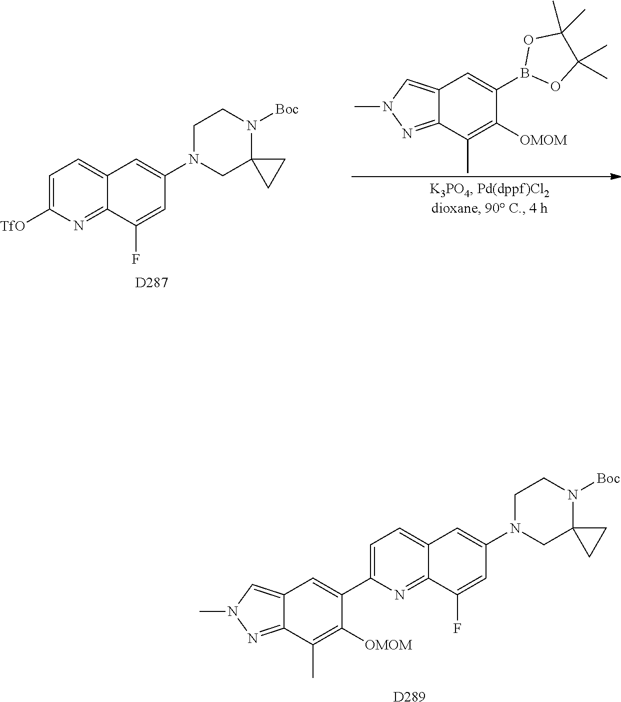

To a stirred solution of tert-butyl 7-[8-fluoro-2-(trifluoromethanesulfonyloxy)quinolin-6-yl]-4,7-diazaspiro[2.5]octane-4-carboxylate (D287, 350 mg, 0.692 mmol, 1.0 equiv) and 6-(methoxymethoxy)-2,7-dimethyl-5-(4,4,5,5-tetramethyl-1,3,2-dioxaborolan-2-yl) indazole (346 mg, 1.038 mmol, 1.5 equiv) in dioxane (6 mL) and H₂O (0.5 mL) were added K₃PO₄ (293.4 mg, 1.384 mmol, 2.0 equiv) and Pd(dppf)Cl₂ (57 mg, 0.069 mmol, 0.10 equiv) at room temperature under nitrogen atmosphere. The resulting mixture was stirred for 4 h at 90° C. under nitrogen atmosphere, allowed to cool to room temperature, and then concentrated under reduced pressure. The residue was purified by silica gel column chromatography and eluted with PE/EA (3:1) to afford tert-butyl 7-{8-fluoro-2-[6-(methoxymethoxy)-2,7-dimethylindazol-5-yl]quinolin-6-yl}-4,7-diazaspiro[2.5]octane-4-carboxylate (D289, 230 mg). LCMS: (ES, m/z): 562 [M+H]⁺.

### Synthesis of Compound 805

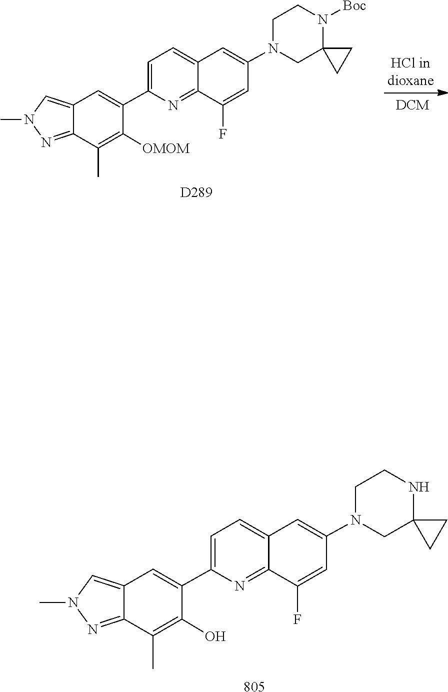

A solution of Intermediate D289 (230 mg, 0.410 mmol, 1.0 equiv) and HCl(gas) in 1,4-dioxane (1 mL) in DCM (6 mL) was stirred for 2 h at room temperature and subsequently concentrated under reduced pressure. The residue was purified by reversed-phase flash chromatography following Condition 10, Gradient 10 as previously described to afford 5-(6-{4,7-diazaspiro[2.5]octan-7-yl}-8-fluoroquinolin-2-yl)-2,7-dimethylindazol-6-ol (Compound 805, 115 mg). LCMS: (ES, m/z): 418 [M+H]⁺. ¹H NMR: (300 MHz, DMSO-d₆) δ 14.51 (s, 1H), 8.49 (s, 1H), 8.36 (d, J=3.1 Hz, 2H), 8.31 (dd, J=9.2, 1.6 Hz, 1H), 7.58 (dd, J=14.4, 2.4 Hz, 1H), 7.05 (d, J=2.4 Hz, 1H), 4.15 (s, 3H), 3.27 (d, J=5.5 Hz, 2H), 3.16 (s, 2H), 2.94 (t, J=5.1 Hz, 2H), 2.40 (s, 1H), 2.38 (s, 3H), 0.57 (t, J=2.3 Hz, 2H), 0.52 (t, J=2.3 Hz, 2H).

### Example 315: Synthesis of Compound 806

### Synthesis of Intermediate D290

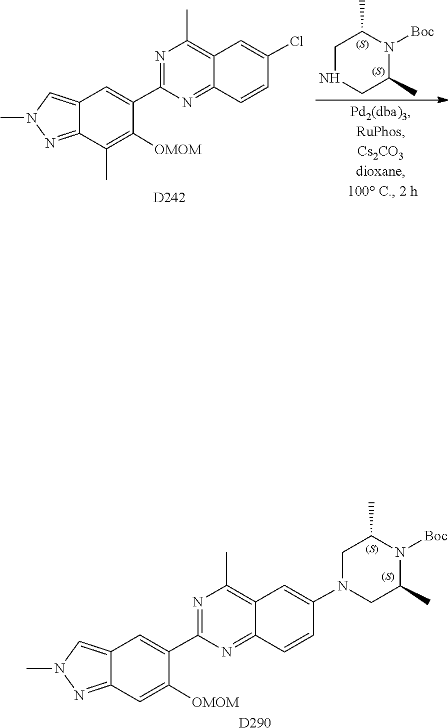

To a stirred solution of 6-chloro-2-[6-(methoxymethoxy)-2-methylindazol-5-yl]-4-methylquinazoline (D242, 200 mg, 0.542 mmol, 1 equiv) and tert-butyl (2S,6S)-2,6-dimethylpiperazine-1-carboxylate (140 mg, 0.650 mmol, 1.2 equiv) in dioxane (4 mL) were added Cs₂CO₃ (353 mg, 1.084 mmol, 2 equiv), RuPhos (51 mg, 0.108 mmol, 0.2 equiv) and Pd₂(dba)₃ (50 mg, 0.054 mmol, 0.1 equiv) at room temperature under nitrogen atmosphere. The resulting mixture was stirred for 2 h at 100° C. under nitrogen atmosphere, allowed to cool to room temperature, and subsequently concentrated under reduced pressure. The residue was purified by silica gel column chromatography and eluted with PE/EA (1:1) to afford tert-butyl (2S,6S)-4-{2-[6-(methoxymethoxy)-2-methylindazol-5-yl]-4-methylquinazolin-6-yl}-2,6-dimethylpiperazine-1-carboxylate (D290, 190 mg). LCMS: (ES, m/z): 547 [M+H]⁺.

### Synthesis of Compound 806

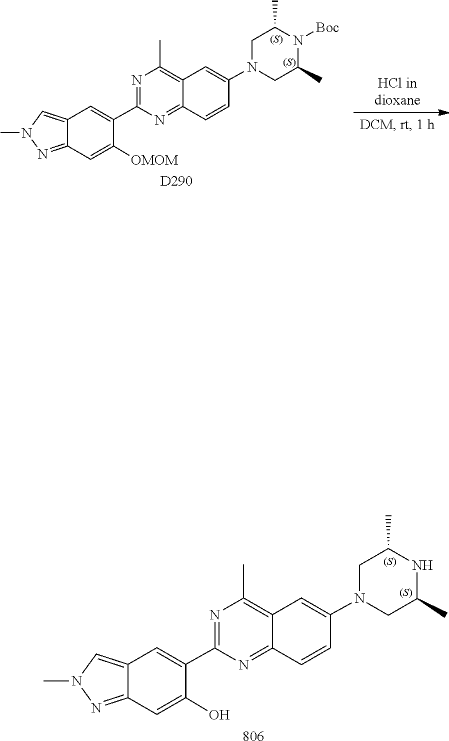

A solution of Intermediate D290 (190 mg, 0.348 mmol, 1 equiv) and 4 M HCl(gas) in 1,4-dioxane (1 mL) in DCM (4 mL) was stirred for 1 h at room temperature and then concentrated under reduced pressure. The residue was purified by reversed-phase flash chromatography following Condition 10, Gradient 17 as previously described to afford 5-{6-[(3S,5S)-3,5-dimethylpiperazin-1-yl]-4-methylquinazolin-2-yl}-2-methylindazol-6-ol (Compound 806, 59.1 mg). Absolute stereochemistry of the compound was arbitrarily assigned. LCMS: (ES, m/z): 403 [M+H]⁺. ¹H NMR: (400 MHz, DMSO-d₆) δ 13.65 (s, 1H), 9.03 (s, 1H), 8.43 (s, 1H), 7.91-7.80 (m, 2H), 7.28 (d, J=2.3 Hz, 1H), 6.88 (s, 1H), 4.12 (s, 3H), 3.40 (dd, J=11.7, 3.4 Hz, 2H), 3.26 (td, J=6.3, 3.4 Hz, 2H), 3.03 (dd, J=11.8, 6.3 Hz, 2H), 2.97 (s, 3H), 1.15 (d, J=6.5 Hz, 6H).

### Example 316: Synthesis of Compound 807

### Synthesis of Intermediate D291

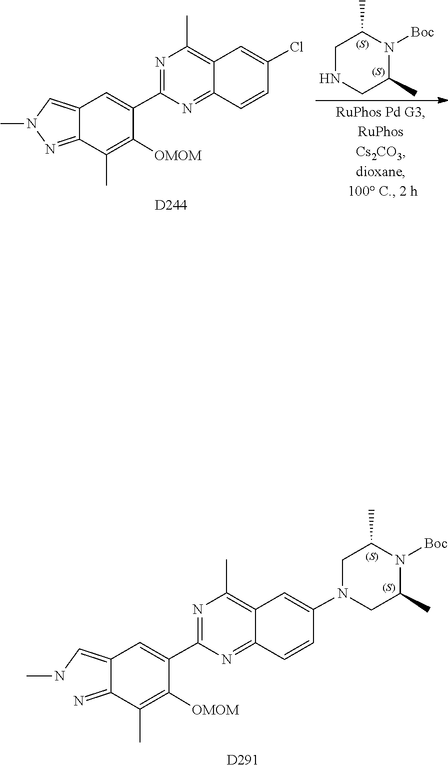

To a solution of 6-chloro-2-[6-(methoxymethoxy)-2,7-dimethylindazol-5-yl]-4-methylquinazoline (D244, 320.0 mg, 0.836 mmol, 1.0 equiv) and tert-butyl (2S,6S)-2,6-dimethylpiperazine-1-carboxylate (268.69 mg, 1.254 mmol, 1.5 equiv) in dioxane (6.0 mL) were added RuPhos Palladacycle Gen.3 (69.91 mg, 0.084 mmol, 0.1 equiv), RuPhos (78.01 mg, 0.167 mmol, 0.2 equiv) and Cs₂CO₃ (816.99 mg, 2.508 mmol, 3.0 equiv). After stirring for 2 h at 100° C. under a nitrogen atmosphere, the resulting mixture was concentrated under reduced pressure. The residue was purified by silica gel column chromatography and eluted with PE/EA (1:1) to afford tert-butyl (2S,6S)-4-{2-[6-(methoxymethoxy)-2,7-dimethylindazol-5-yl]-4-methylquinazolin-6-yl}-2,6-dimethylpiperazine-1-carboxylate (D291, 200.0 mg). LCMS: (ES, m/z): 561 [M+H]⁺.

### Synthesis of Compound 807

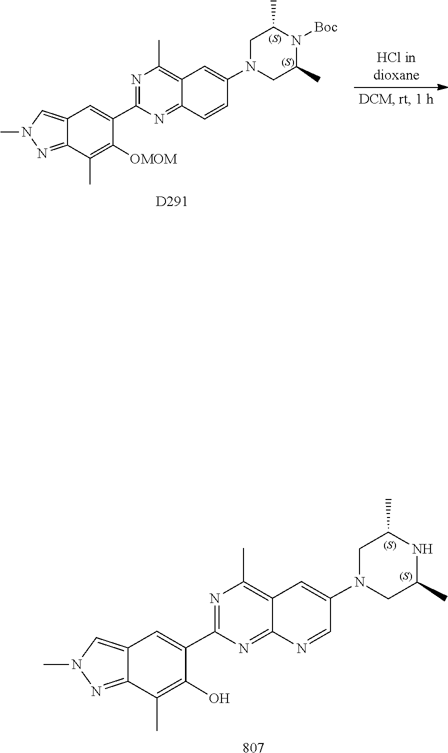

To a stirred solution of Intermediate D291 (200.0 mg, 0.357 mmol, 1.0 equiv) in DCM (3.0 mL) was added HCl(gas) in 1,4-dioxane (0.53 mL, 17.568 mmol, 49.21 equiv) dropwise at 0° C. The resulting mixture was stirred for 1 h at room temperature and subsequently concentrated under reduced pressure. The crude product was purified by Prep-HPLC following Condition 19, Gradient 1 as previously described to afford 5-{6-[(3S,5S)-3,5-dimethylpiperazin-1-yl]-4-methylquinazolin-2-yl}-2,7-dimethylindazol-6-ol (Compound 807, 100.0 mg). Absolute stereochemistry of the compound was arbitrarily assigned. LCMS: (ES, m/z): 417 [M+H]⁺. ¹H NMR: (400 MHz, DMSO-d₆) δ 13.83 (s, 1H), 8.90 (d, J=0.8 Hz, 1H), 8.40 (s, 1H), 7.92-7.81 (m, 2H), 7.28 (d, J=2.4 Hz, 1H), 4.14 (s, 3H), 3.39 (dd, J=11.7, 3.4 Hz, 2H), 3.27-3.21 (m, 2H), 3.02 (dd, J=11.7, 6.2 Hz, 2H), 2.97 (s, 3H), 2.39 (s, 3H), 1.14 (d, J=6.4 Hz, 6H).

### Example 317: Synthesis of Compounds 808 and 809

### Synthesis of Intermediate D293

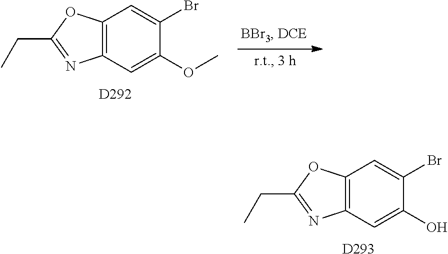

A solution of 6-bromo-2-ethyl-5-methoxy-1,3-benzoxazole (D292, 700 mg, 2.733 mmol, 1 equiv) in DCE (10 mL) was treated with BBr₃ (700 uL, 7.405 mmol, 2.71 equiv) in DCE (4 mL) for 3 h at room temperature. The reaction was quenched with MeOH (10 mL) at 0° C., basified to pH 10 with saturated Na₂CO₃ (aq.), and the aqueous layer was extracted with EtOAc (3×3 mL). The combined organic layers were dried over anhydrous Na₂SO₄, and the filtrate was concentrated under reduced pressure. This resulted in 6-bromo-2-ethyl-1,3-benzoxazol-5-ol (D293, 600 mg). LCMS:(ESI, m/z):242 [M+H]⁺.

### Synthesis of Intermediate D294

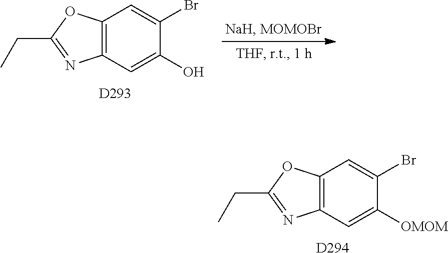

To a stirred solution of Intermediate D293 (500 mg, 2.066 mmol, 1 equiv) in THF (2 mL) was added NaH (247.84 mg, 6.198 mmol, 3 equiv, 60%) in portions at 0° C. and then stirred for 30 min at room temperature. To the above mixture was added bromomethoxymethane (309.74 mg, 2.479 mmol, 1.2 equiv) and stirred for an additional 1 h at room temperature, before quenching with water (10 mL) at 0° C. The aqueous layer was extracted with EtOAc (3×5 mL), and the combined organic layers were dried over anhydrous Na₂SO₄. After filtration, the filtrate was concentrated under reduced pressure. The residue was purified by silica gel column chromatography and eluted with PE/EA (10:1) to afford 6-bromo-2-ethyl-5-(methoxymethoxy)-1,3-benzoxazole (D294, 500 mg). LCMS:(ESI, m/z):286 [M+H]⁺.

### Synthesis of Intermediate D295

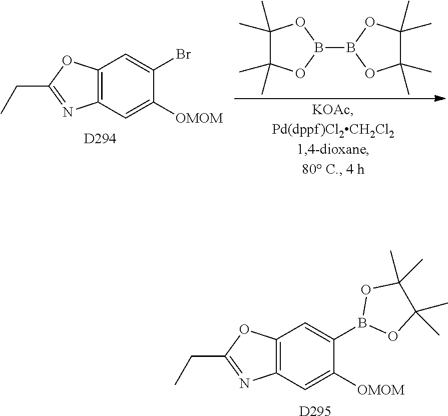

A solution of Intermediate D294 (500 mg, 1.747 mmol, 1 equiv) in dioxane (15 mL) was treated with bis(pinacolato)diboron (887.51 mg, 3.494 mmol, 2 equiv), KOAc (514.51 mg, 5.241 mmol, 3 equiv) and Pd(dppf)Cl₂·CH₂Cl₂ (142.35 mg, 0.175 mmol, 0.1 equiv) for 4 h at 80° C. under nitrogen atmosphere. The resulting mixture was filtered, washed with EtOAc (3×5 mL), and concentrated under reduced pressure. The residue was purified by silica gel column chromatography and eluted with PE/EA (7:1) to afford 2-ethyl-5-(methoxymethoxy)-6-(4,4,5,5-tetramethyl-1,3,2-dioxaborolan-2-yl)-1,3-benzoxazole (D295, 300 mg). LCMS:(ESI, m/z):334[M+H]⁺

### Synthesis of Intermediate D296

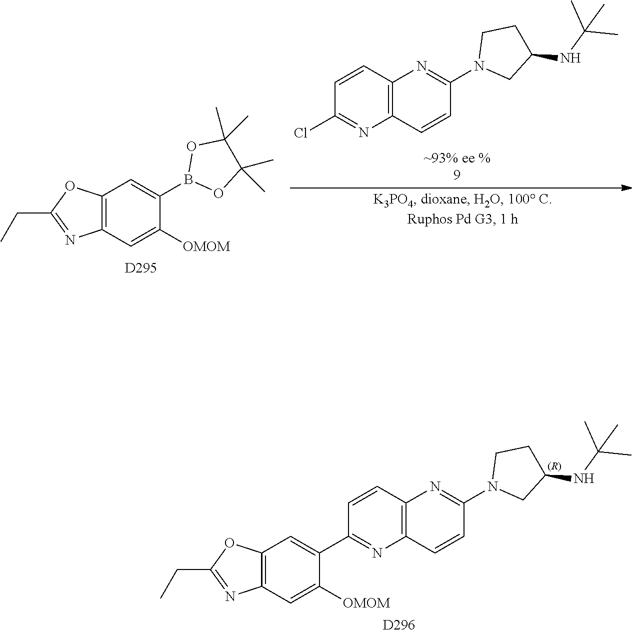

A solution of Intermediate D295 (10 mg, 0.030 mmol, 1 equiv) in dioxane (1 mL)/H₂O (0.2 mL) was treated with (3R)-N-tert-butyl-1-(6-chloro-1,5-naphthyridin-2-yl)pyrrolidin-3-amine (9.15 mg, 0.030 mmol, 1 equiv), K₃PO₄ (19.11 mg, 0.090 mmol, 3 equiv) and RuPhos Palladacycle Gen.3 (2.51 mg, 0.003 mmol, 0.1 equiv) for 1 h at 100° C. under nitrogen atmosphere. The aqueous layer was extracted with EtOAc (3×2 mL), the combined organic layers were dried over anhydrous Na₂SO₄, and the filtrate was concentrated under reduced pressure. The residue was purified by Prep-TLC (PE/EA 1:1) to afford (3R)-N-tert-butyl-1-{6-[2-ethyl-5-(methoxymethoxy)-1,3-benzoxazol-6-yl]-1,5-naphthyridin-2-yl}pyrrolidin-3-amine (D296, 220 mg) as a yellow solid. LCMS:(ESI, m/z): 476[M+H]⁺.

### Synthesis of Compound 808

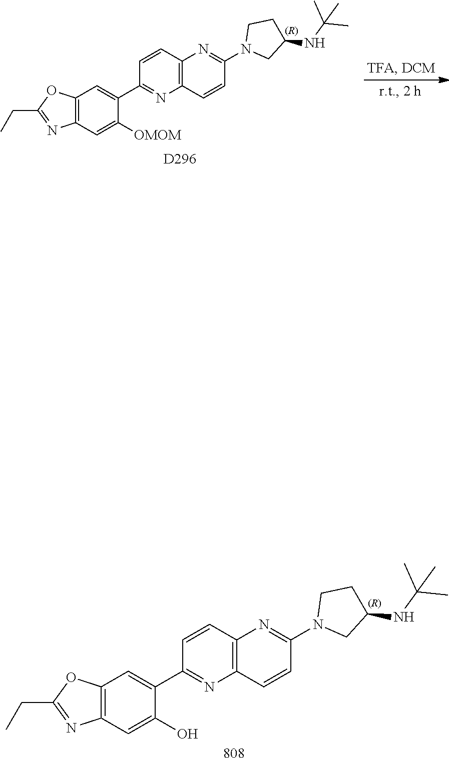

A solution of Intermediate D296 (10 mg, 0.021 mmol, 1 equiv) in TFA (0.3 mL) DCM (1 mL) was stirred for 2 h at room temperature and subsequently concentrated under reduced pressure. The crude product was purified by Chiral-Prep-HPLC following Condition 12, Gradient 2 as previously described to afford 6-{6-[(3R)-3-(tert-butylamino)pyrrolidin-1-yl]-1,5-naphthyridin-2-yl}-2-ethyl-1,3-benzoxazol-5-ol (60 mg), then the product was purified by Chiral-Prep-HPLC following Condition 4, Gradient 15 as previously described to afford 6-{6-[(3R)-3-(tert-butylamino)pyrrolidin-1-yl]-1,5-naphthyridin-2-yl}-2-ethyl-1,3-benzoxazol-5-ol (Compound 808, 39.4 mg). Absolute stereochemistry of the compound was arbitrarily assigned. LCMS: (ESI, m/z): 432[M+H]⁺. ¹H NMR:(400 MHz, DMSO-d₆) δ 14.55 (s, 1H), 8.44-8.37 (m, 2H), 8.08 (ddd, J=12.5, 9.2, 0.8 Hz, 2H), 7.14 (t, J=4.6 Hz, 2H), 3.85 (s, 1H), 3.70 (s, 1H), 3.49 (ddd, J=18.9, 12.5, 7.3 Hz, 2H), 3.16-3.07 (m, 1H), 2.96 (q, J=7.5 Hz, 2H), 2.17 (ddt, J=9.8, 6.5, 3.5 Hz, 1H), 1.81-1.70 (m, 1H), 1.35 (t, J=7.5 Hz, 3H), 1.09 (s, 9H).

### Synthesis of Intermediate D297

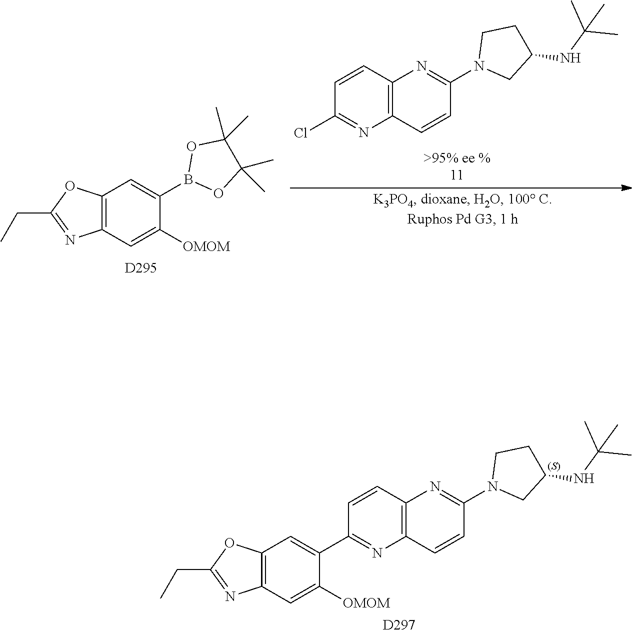

A solution of Intermediate D295 (71.05 mg, 0.213 mmol, 1 equiv) in dioxane (5 mL)/H₂O (1 mL) was treated with (3S)—N-tert-butyl-1-(6-chloro-1,5-naphthyridin-2-yl)pyrrolidin-3-amine (65 mg, 0.213 mmol, 1.00 equiv), K₃PO₄ (135.79 mg, 0.639 mmol, 3 equiv) and RuPhos Palladacycle Gen.3 (17.84 mg, 0.021 mmol, 0.1 equiv) for 1 h at 100° C. under nitrogen atmosphere. The resulting mixture was filtered and washed with EtOAc (3×2 mL). The aqueous layer was extracted with EtOAc (3×2 mL), the combined organic layers were dried over anhydrous Na₂SO₄, and the filtrate was concentrated under reduced pressure. The residue was purified by Prep-TLC (PE/EA 1:1) to afford (3S)—N-tert-butyl-1-{6-[2-ethyl-5-(methoxymethoxy)-1,3-benzoxazol-6-yl]-1,5-naphthyridin-2-yl}pyrrolidin-3-amine (D297, 80 mg). LCMS: (ESI, m/z): 476[M+H]⁺.

### Synthesis of Compound 809

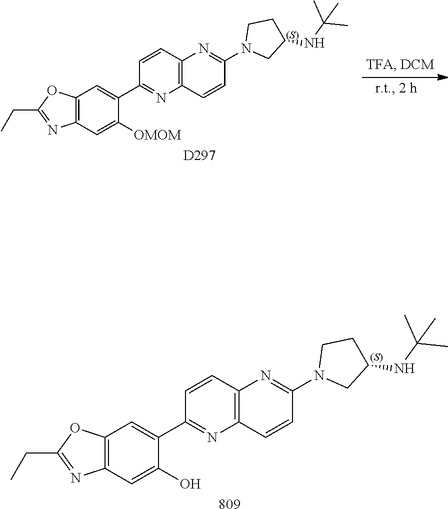

A solution of Intermediate D297 (70 mg, 0.147 mmol, 1 equiv) in TFA (1.2 mL)/DCM (3 mL) was stirred for 2 h at room temperature and subsequently concentrated under reduced pressure. The crude product was purified by Chiral-Prep-HPLC following Condition 21, Gradient 1 as previously described to afford 6-{6-[(3S)-3-(tert-butylamino)pyrrolidin-1-yl]-1,5-naphthyridin-2-yl}-2-ethyl-1,3-benzoxazol-5-ol (Compound 809, 34.7 mg). Absolute stereochemistry of the compound was arbitrarily assigned. LCMS:(ESI, m/z):432[M+H]⁺. ¹H NMR:(400 MHz, DMSO-d₆) δ 14.53 (s, 1H), 8.43-8.37 (m, 2H), 8.08 (dd, J=12.1, 9.2 Hz, 2H), 7.16-7.11 (m, 2H), 3.85 (s, 1H), 3.70 (s, 1H), 3.55-3.44 (m, 2H), 3.12 (t, J=9.0 Hz, 1H), 2.95 (q, J=7.5 Hz, 2H), 2.22-2.12 (m, 1H), 1.82-1.67 (m, 2H), 1.35 (t, J=7.5 Hz, 3H), 1.09 (s, 9H).

### Example 318: Synthesis of Compound 810

### Synthesis of Intermediate D298

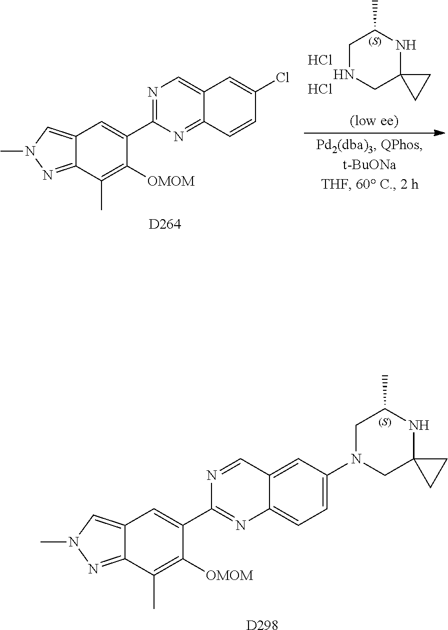

To a solution of 6-chloro-2-[6-(methoxymethoxy)-2,7-dimethylindazol-5-yl]quinazoline (D264, 200 mg, 0.542 mmol, 1 equiv) and (5S)-5-methyl-4,7-diazaspiro[2.5]octane dihydrochloride (100 mg, 0.502 mmol, 0.93 equiv) in THF (5 mL) were added t-BuONa (208.4 mg, 2.168 mmol, 4.0 equiv), 1,2,3,4,5-pentaphenyl-1-(di-tert-butylphosphino)ferrocene (77 mg, 0.108 mmol, 0.2 equiv) and Pd₂(dba)₃ (50 mg, 0.054 mmol, 0.1 equiv). After stirring for 2 h at 60° C. under a nitrogen atmosphere, the resulting mixture was concentrated under reduced pressure. The residue was purified by reversed-phase flash chromatography following Condition 10, Gradient 4 as previously described to afford 2-[6-(methoxymethoxy)-2,7-dimethylindazol-5-yl]-6-[(5S)-5-methyl-4,7-diazaspiro[2.5]octan-7-yl]quinazoline (D298, 175 mg). LCMS: (ES, m/z): 459[M+H]⁺.

### Synthesis of Compound 810

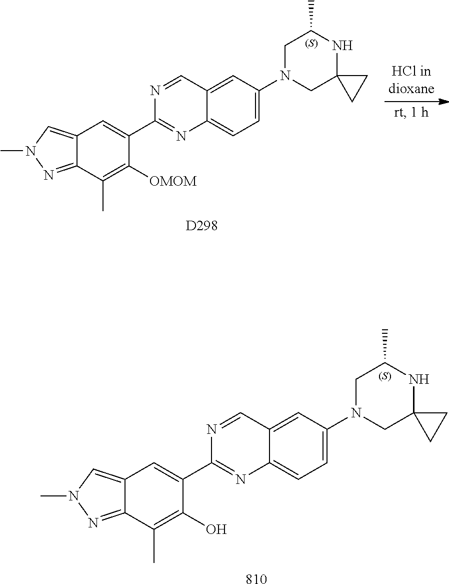

To a stirred solution of Intermediate D298 (175 mg, 0.382 mmol, 1 equiv) in DCM (2 mL) were added HCl (gas) in 1,4-dioxane (1 mL, 4M) dropwise at room temperature. The resulting mixture was stirred for 1 h and subsequently concentrated under reduced pressure. The residue was purified by reversed-phase flash chromatography following Condition 10, Gradient 8 as previously described to afford 2,7-dimethyl-5-(6-(5-methyl-4,7-diazaspiro[2.5]octan-7-yl)quinazolin-2-yl)-2H-indazol-6-ol (120 mg) as a solid, which was further separated by Prep-Chiral-HPLC following Condition 20, Gradient 1 as previously described to afford 2,7-dimethyl-5-{6-[(5S)-5-methyl-4,7-diazaspiro[2.5]octan-7-yl]quinazolin-2-yl}indazol-6-ol (Compound 810, 36 mg). Absolute stereochemistry of the compound was arbitrarily assigned. RT=4.373 min on chiral-HPLC. LCMS: (ES, m/z):415 [M+H]⁺. ¹H NMR: (300 MHz, DMSO-d₆) δ 13.64 (s, 1H), 9.50 (s, 1H), 8.90 (s, 1H), 8.40 (s, 1H), 7.91 (d, J=1.4 Hz, 2H), 7.34 (s, 1H), 4.14 (s, 3H), 3.90-3.80 (m, 1H), 3.16 (q, J=12.0 Hz, 2H), 3.00 (s, 1H), 2.45 (d, J=11.1 Hz, 1H), 2.40 (s, 3H), 1.07 (d, J=6.4 Hz, 3H), 0.57 (dq, J=15.9, 7.2 Hz, 3H), 0.46 (d, J=7.7 Hz, 1H).

### Example 319: Synthesis of Compound 811

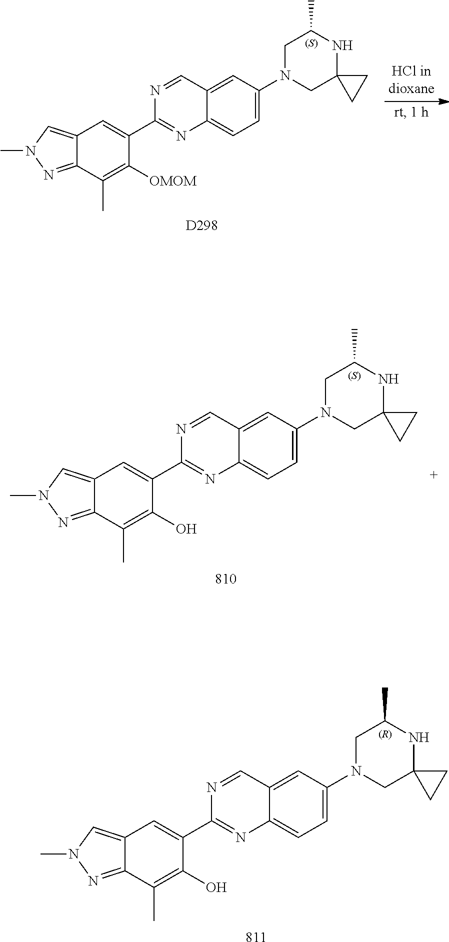

To a stirred solution of 2-[6-(methoxymethoxy)-2,7-dimethylindazol-5-yl]-6-[(5S)-5-methyl-4,7-diazaspiro[2.5]octan-7-yl]quinazoline (D298, 175 mg, 0.382 mmol, 1 equiv) in DCM (2 mL) were added HCl (gas) in 1,4-dioxane (1 mL, 4M) dropwise at room temperature, stirred for 1 h, and then concentrated under reduced pressure. The residue was purified by reversed-phase flash chromatography following Condition 10, Gradient 8 as previously described to afford 2,7-dimethyl-5-(6-(5-methyl-4,7-diazaspiro[2.5]octan-7-yl)quinazolin-2-yl)-2H-indazol-6-ol (120 mg) as a solid, which was further separated by Prep-Chiral-HPLC following Condition 20, Gradient 1 to afford 2,7-dimethyl-5-{6-[(5R)-5-methyl-4,7-diazaspiro[2.5]octan-7-yl]quinazolin-2-yl}indazol-6-ol (Compound 811, 20 mg). Absolute stereochemistry of the compound was arbitrarily assigned. RT=4.980 min on chiral-HPLC. LCMS: (ES, m/z):415 [M+H]⁺. ¹H NMR: (300 MHz, DMSO-d₆) δ 13.64 (s, 1H), 9.50 (s, 1H), 8.90 (d, J=0.8 Hz, 1H), 8.40 (s, 1H), 7.91 (d, J=1.5 Hz, 2H), 7.34 (s, 1H), 4.14 (s, 3H), 3.85 (d, J=10.5 Hz, 1H), 3.16 (q, J=12.0 Hz, 2H), 3.00 (s, 1H), 2.50-2.37 (m, 4H), 2.18 (s, 1H), 1.07 (d, J=6.4 Hz, 3H), 0.58 (td, J=12.0, 9.9, 6.9 Hz, 3H), 0.46 (d, J=7.4 Hz, 1H).

### Example 320: Synthesis of Compound 812

### Synthesis of Intermediate D299

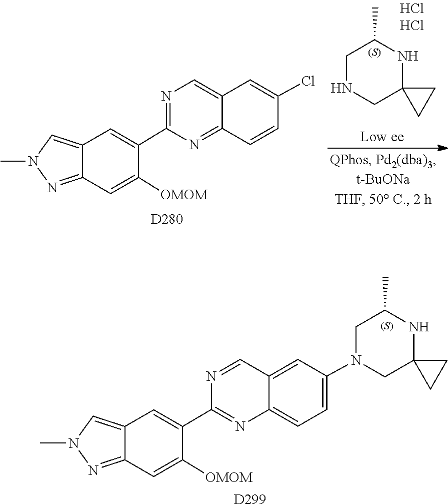

To a solution of 6-chloro-2-[6-(methoxymethoxy)-2-methylindazol-5-yl]quinazoline (D280, 230 mg, 0.648 mmol, 1.0 equiv) and 5-methyl-4,7-diazaspiro[2.5]octane dihydrochloride (155 mg, 0.778 mmol, 1.2 equiv) in THF (3.0 mL) were added Pd₂(dba)₃ (59 mg, 0.065 mmol, 0.1 equiv), Qphos (92 mg, 0.130 mmol, 0.2 equiv) and t-BuONa (125 mg, 1.296 mmol, 2.0 equiv). After stirring for 2 h at 50° C. under a nitrogen atmosphere, the resulting mixture was concentrated under reduced pressure. The residue was purified by silica gel column chromatography and eluted with CH₂Cl₂/MeOH (10:1) to afford 2-(6-(methoxymethoxy)-2-methyl-2H-indazol-5-yl)-6-(5-methyl-4,7-diazaspiro[2.5]octan-7-yl)quinazoline (D299, 200.0 mg). LCMS: (ES, m/z): 445 [M+H]⁺.

### Synthesis of Compound 812

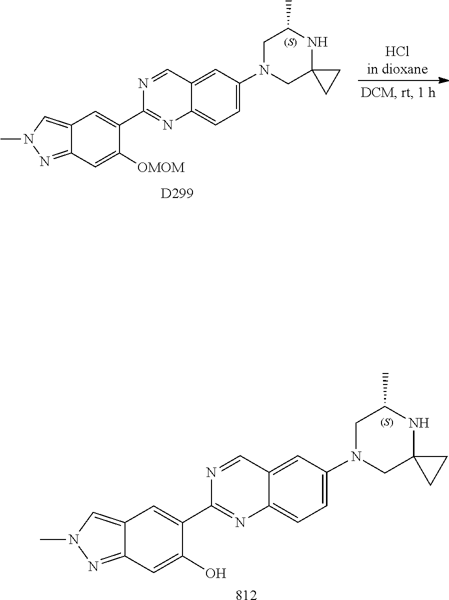

To a stirred solution of Intermediate D299 (200 mg, 0.450 mmol, 1 equiv) in DCM (3.0 mL) was added HCl(gas) in 1,4-dioxane (0.2 mL) dropwise at 0° C. The resulting mixture was stirred for 1 h at room temperature and then concentrated under reduced pressure. The crude product was purified by Prep-HPLC following Condition 19, Gradient as previously described and separated by prep-chiral-HPLC following Condition 19, Gradient 1 as previously described to afford(S)-2-methyl-5-(6-(5-methyl-4,7-diazaspiro[2.5]octan-7-yl)quinazolin-2-yl)-2H-indazol-6-ol (Compound 812, 23.0 mg). Absolute stereochemistry of the compound was arbitrarily assigned. LCMS: (ES, m/z): 401 [M+H]⁺. ¹H NMR: (400 MHz, DMSO-d₆) δ 13.46 (s, 1H), 9.51 (s, 1H), 9.04 (s, 1H), 8.43 (s, 1H), 7.92 (d, J=2.2 Hz, 2H), 7.36 (d, J=2.0 Hz, 1H), 6.89 (s, 1H), 4.12 (s, 3H), 3.90-3.82 (m, 1H), 3.21 (d, J=11.9 Hz, 1H), 3.13 (d, J=11.8 Hz, 1H), 3.01 (s, 1H), 2.44 (t, J=11.1 Hz, 1H), 2.12 (d, J=30.3 Hz, 1H), 1.08 (d, J=6.5 Hz, 3H), 0.65-0.52 (m, 3H), 0.46 (s, 1H).

### Example 321: Synthesis of Compound 813

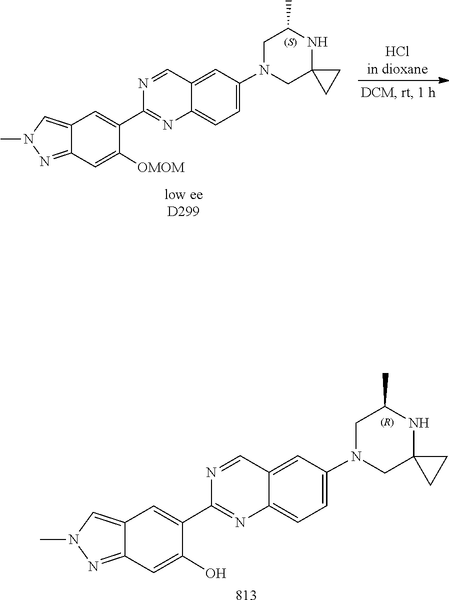

To a stirred solution of 2-(6-(methoxymethoxy)-2-methyl-2H-indazol-5-yl)-6-(5-methyl-4,7-diazaspiro[2.5]octan-7-yl)quinazoline (D299, 200.0 mg, 0.450 mmol, 1 equiv) in DCM (3.0 mL) was added HCl(gas) in 1,4-dioxane (0.2 mL, 6.583 mmol, 14.63 equiv) dropwise at 0° C. The resulting mixture was stirred for 1 h at room temperature and then concentrated under reduced pressure. The crude product was purified by Prep-HPLC following Condition 19, Gradient 1 as previously described to afford 2-methyl-5-(6-(5-methyl-4,7-diazaspiro[2.5]octan-7-yl)quinazolin-2-yl)-2H-indazol-6-ol (50.0 mg, 25.43%) as a solid, which was further separated by prep-chiral-HPLC following Condition 19, Gradient 1 as previously described (to afford (R)-2-methyl-5-(6-(5-methyl-4,7-diazaspiro[2.5]octan-7-yl)quinazolin-2-yl)-2H-indazol-6-ol (Compound 813, 14.0 mg). Absolute stereochemistry of the compound was arbitrarily assigned. LCMS: (ES, m/z): 401 [M+H]⁺. ¹H NMR: (400 MHz, DMSO-d₆) δ 13.46 (s, 1H), 9.51 (s, 1H), 9.04 (s, 1H), 8.43 (s, 1H), 7.91 (d, J=2.2 Hz, 2H), 7.36 (d, J=1.9 Hz, 1H), 6.89 (s, 1H), 4.12 (s, 3H), 3.90-3.82 (m, 1H), 3.19 (s, 1H), 3.13 (d, J=11.9 Hz, 1H), 3.01 (s, 1H), 2.48-2.40 (m, 1H), 2.15 (s, 1H), 1.07 (d, J=6.5 Hz, 3H), 0.65-0.52 (m, 3H), 0.46 (d, J=7.8 Hz, 1H).

### Example 322: Synthesis of Compounds 814 and 815

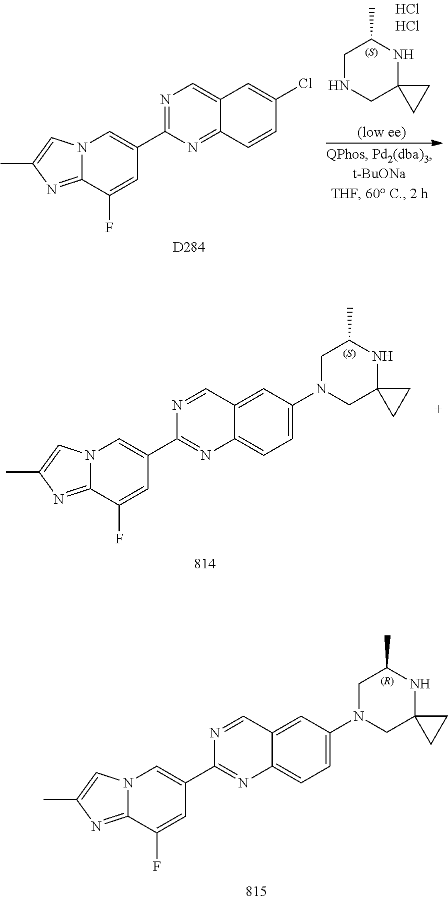

To a stirred mixture of 6-chloro-2-{8-fluoro-2-methylimidazo[1,2-a]pyridin-6-yl}quinazoline (D284, 200 mg, 0.640 mmol, 1 equiv) and (5S)-5-methyl-4,7-diazaspiro[2.5]octane (96.8 mg, 0.768 mmol, 1.2 equiv) in THF (5 mL) were added t-BuONa (184.4 mg, 1.920 mmol, 3 equiv), Q-Phos (90.6 mg, 0.128 mmol, 0.2 equiv) and Pd₂(dba)₃ (58 mg, 0.064 mmol, 0.1 equiv) at room temperature under nitrogen atmosphere. The resulting mixture was stirred for 2 h at 60° C. under nitrogen atmosphere and then concentrated under reduced pressure. The residue was purified by reversed-phase flash chromatography following Condition 10, Gradient 16 as previously described to yield 90 mg of the target compound. The target compound was separated by chiral-prep-HPLC following Condition 18, Gradient 1 as previously described to afford 2-{8-fluoro-2-methylimidazo[1,2-a]pyridin-6-yl}-6-[(5S)-5-methyl-4,7-diazaspiro[2.5]octan-7-yl]quinazoline (Compound 814, 35.4 mg) and 2-{8-fluoro-2-methylimidazo[1,2-a]pyridin-6-yl}-6-[(5R)-5-methyl-4,7-diazaspiro[2.5]octan-7-yl]quinazoline (Compound 815, 23.8 mg). Absolute stereochemistry of the compounds was arbitrarily assigned. Compound 814: LCMS: (ES, m/z): 403 [M+H]⁺. RT=1.741 min on chiral-SFC. ¹H NMR: (300 MHz, DMSO-d₆) δ 9.43 (d, J=1.3 Hz, 2H), 8.01 (d, J=3.1 Hz, 1H), 7.95 (dd, J=12.6, 1.3 Hz, 1H), 7.88 (d, J=2.8 Hz, 2H), 7.31 (d, J=2.1 Hz, 1H), 3.84 (d, J=10.9 Hz, 1H), 3.19 (d, J=12.0 Hz, 1H), 3.12 (d, J=12.0 Hz, 1H), 3.01 (s, 1H), 2.45 (d, J=11.1 Hz, 1H), 2.39 (s, 3H), 1.07 (d, J=6.4 Hz, 3H), 0.67-0.65 (m, 3H), 0.50-0.42 (m, 1H). Compound 815: LCMS: (ES, m/z) 403 [M+H]⁺. RT=1.629 min on chiral-SFC. ¹H NMR: (300 MHz, DMSO-d₆) δ 9.43 (d, J=1.3 Hz, 2H), 8.01 (d, J=3.1 Hz, 1H), 7.95 (dd, J=12.6, 1.3 Hz, 1H), 7.88 (d, J=2.8 Hz, 2H), 7.31 (d, J=2.1 Hz, 1H), 3.84 (d, J=10.9 Hz, 1H), 3.19 (d, J=12.0 Hz, 1H), 3.12 (d, J=12.0 Hz, 1H), 3.01 (s, 1H), 2.45 (d, J=11.1 Hz, 1H), 2.39 (s, 3H), 1.07 (d, J=6.4 Hz, 3H), 0.62 (d, J=6.7 Hz, 1H), 0.61-0.42 (m, 3H).

### Example 323: Synthesis of Compound 818

### Synthesis of Intermediate D301

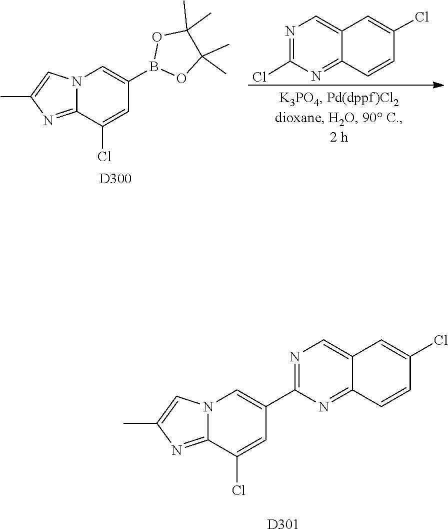

To a stirred solution of 8-chloro-2-methyl-6-(4,4,5,5-tetramethyl-1,3,2-dioxaborolan-2-yl)imidazo[1,2-a]pyridine (D300, 200 mg, 0.684 mmol, 1 equiv) and 2,6-dichloroquinazoline (163 mg, 0.821 mmol, 1.2 equiv) in dioxane (5 mL) and H₂O (0.5 mL) were added K₃PO₄ (290 mg, 1.368 mmol, 2 equiv) and Pd(dppf)Cl₂ (50 mg, 0.068 mmol, 0.1 equiv) at room temperature. The resulting mixture was stirred for 2 h at 90° C. under nitrogen atmosphere and then concentrated under reduced pressure. The residue was purified by silica gel column chromatography and eluted with PE/EA (1:10) to afford 6-chloro-2-{8-chloro-2-methylimidazo[1,2-a]pyridin-6-yl}quinazoline (D301, 180 mg). LCMS: (ES, m/z): 329 [M+H]⁺.

### Synthesis of Intermediate D302

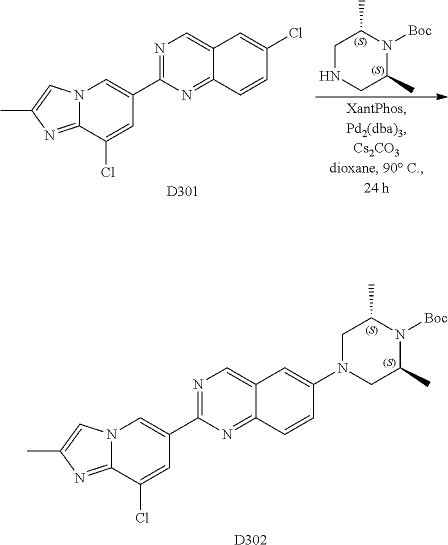

To a stirred solution of Intermediate D301 (160 mg, 0.486 mmol, 1 equiv) and tert-butyl (2S,6S)-2,6-dimethylpiperazine-1-carboxylate (125 mg, 0.583 mmol, 1.2 equiv) in dioxane (5 mL) were added XantPhos (56 mg, 0.097 mmol, 0.2 equiv), Cs₂CO₃ (475 mg, 1.458 mmol, 3 equiv) and Pd₂(dba)₃ (45 mg, 0.049 mmol, 0.1 equiv) at room temperature and stirred for 24 h at 90° C. under nitrogen atmosphere. The residue was purified by silica gel column chromatography and eluted with PE/EA (1:10) to afford tert-butyl (2S,6S)-4-(2-{8-chloro-2-methylimidazo[1,2-a]pyridin-6-yl}quinazolin-6-yl)-2,6-dimethylpiperazine-1-carboxylate (D302, 25 mg). LCMS: (ES, m/z): 507 [M+H]⁺.

### Synthesis of Compound 818

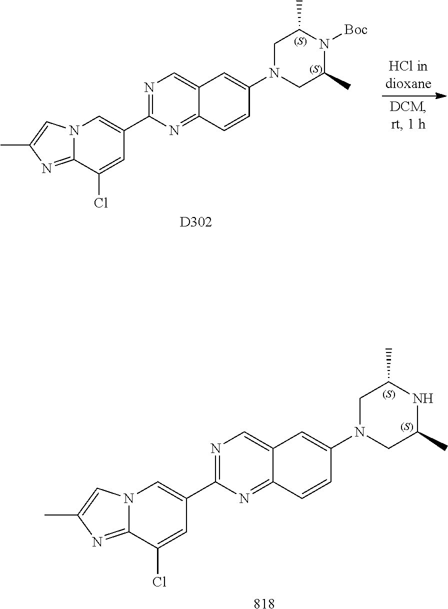

To a stirred mixture of Intermediate D302 (25 mg, 0.049 mmol, 1 equiv) in DCM (1 mL) was added HCl(gas) in 1,4-dioxane (0.2 mL, 4 M) dropwise at room temperature. The resulting mixture was stirred for 1 h and subsequently concentrated under vacuum. The crude product was purified by Prep-HPLC following Condition 6, Gradient 3 to afford 2-{8-chloro-2-methylimidazo[1,2-a]pyridin-6-yl}-6-[(3S,5S)-3,5-dimethylpiperazin-1-yl]quinazoline (Compound 818, 3 mg). Absolute stereochemistry of the compound was arbitrarily assigned. LCMS: (ES, m/z): 407 [M+H]⁺. RT=5.989 min on chiral-SFC. ¹H NMR: (300 MHz, DMSO-d₆) δ 9.55 (d, J=1.5 Hz, 1H), 9.45 (s, 1H), 8.31 (d, J=1.5 Hz, 1H), 8.02 (d, J=1.1 Hz, 1H), 7.89 (s, 2H), 7.34 (s, 1H), 3.38 (m, 2H), 3.27 (m, 2H), 3.03 (dd, J=11.7, 6.2 Hz, 2H), 2.40 (d, J=0.9 Hz, 3H), 1.14 (d, J=6.4 Hz, 6H).

### Example 324: Synthesis of Compound 819

### Synthesis of Intermediate D304

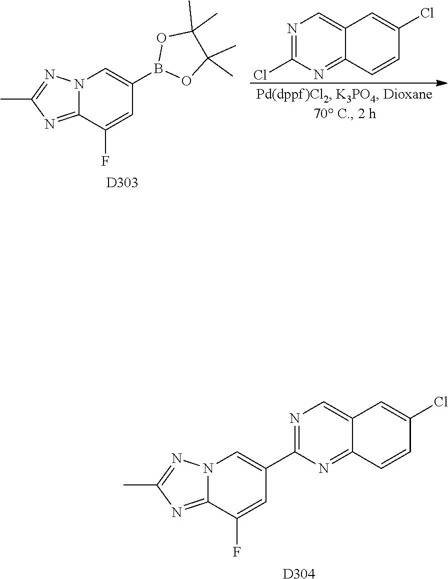

To a solution of 8-fluoro-2-methyl-6-(4,4,5,5-tetramethyl-1,3,2-dioxaborolan-2-yl)-[1,2,4]triazolo[1,5-a]pyridine (D303, 200 mg, 0.722 mmol, 1.0 equiv) and 2,6-dichloroquinazoline (144 mg, 0.722 mmol, 1 equiv) in dioxane (4 mL) and H₂O (1 mL) were added K₃PO₄ (306 mg, 1.444 mmol, 2.0 equiv) and Pd(dppf)Cl₂ (53 mg, 0.072 mmol, 0.1 equiv). After stirring for 2 h at 70° C. under a nitrogen atmosphere, the resulting mixture was concentrated under reduced pressure. The residue was purified by silica gel column chromatography and eluted with PE/EA (1:1) to afford 6-chloro-2-{8-fluoro-2-methyl-[1,2,4]triazolo[1,5-a]pyridin-6-yl}quinazoline (D304, 200 mg). LCMS: (ES, m/z): 314 [M+H]⁺.

### Synthesis of Intermediate D305

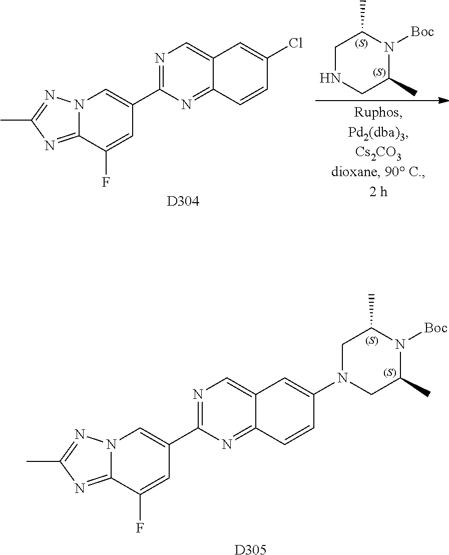

To a solution of Intermediate D304 (200 mg, 0.638 mmol, 1.0 equiv) and tert-butyl (2S,6S)-2,6-dimethylpiperazine-1-carboxylate (178 mg, 0.829 mmol, 1.3 equiv) in dioxane (5 mL) were added Cs₂CO₃ (415 mg, 1.276 mmol, 2.0 equiv), Pd₂(dba)₃ (58 mg, 0.064 mmol, 0.1 equiv), Ruphos (60 mg, 0.128 mmol, 0.2 equiv). After stirring for 2 h at 90° C. under a nitrogen atmosphere, the resulting mixture was concentrated under reduced pressure. The residue was purified by silica gel column chromatography and eluted with PE/EA (1:2) to afford tert-butyl (2S,6S)-4-(2-{8-fluoro-2-methyl-[1,2,4]triazolo[1,5-a]pyridin-6-yl}quinazolin-6-yl)-2,6-dimethylpiperazine-1-carboxylate (D305, 190 mg). LCMS: (ES, m/z): 492[M+H]⁺.

### Synthesis of Compound 819

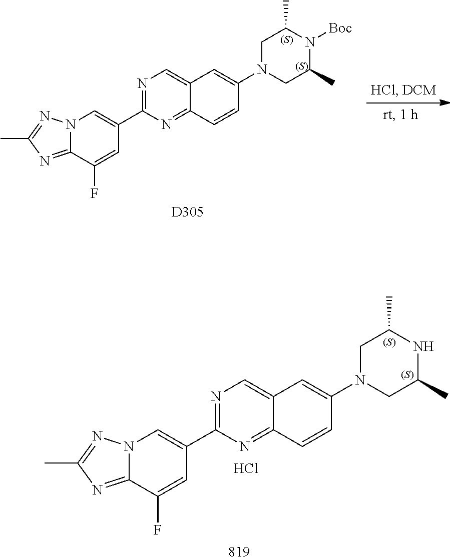

To a stirred mixture of Intermediate D305 (200 mg, 0.407 mmol, 1.0 equiv) in DCM (4 mL) was added HCl(gas) in 1,4-dioxane (2 mL) at room temperature and stirred for 1 h. The reaction, monitoring by LCMS. The resulting mixture was concentrated under reduced pressure and diluted with MeOH (4 mL) and H₂O (1 ml). The precipitated solids were collected by filtration and washed with water (1×1 mL) to afford 6-[(3S,5S)-3,5-dimethylpiperazin-1-yl]-2-{8-fluoro-2-methyl-[1,2,4]triazolo[1,5-a]pyridin-6-yl}quinazoline hydrochloride (Compound 819, 81 mg). Absolute stereochemistry of the compound was arbitrarily assigned. LCMS: (ES, m/z): 392[M+H]⁺. ¹H NMR: (400 MHz, DMSO-d₆) δ 9.51 (t, J=3.1 Hz, 2H), 9.34 (s, 2H), 8.44-8.35 (m, 1H), 8.03-7.92 (m, 2H), 7.50 (d, J=2.4 Hz, 1H), 3.77-3.62 (m, 4H), 3.40 (dd, J=13.2, 6.5 Hz, 2H), 2.56 (s, 3H), 1.39 (d, J=6.5 Hz, 6H).

### Example 325: Synthesis of Compound 820

### Synthesis of Intermediate D307

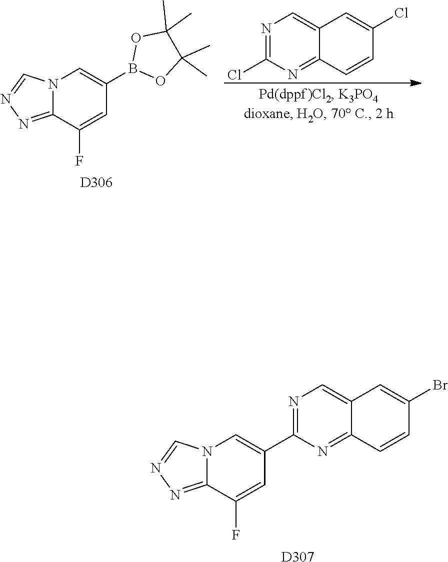

To a solution of 2,6-dichloroquinazoline (252.1 mg, 1.267 mmol, 1.0 equiv) and 8-fluoro-6-(4,4,5,5-tetramethyl-1,3,2-dioxaborolan-2-yl)-[1,2,4]triazolo[4,3-a]pyridine (D306, 500.0 mg, 1.901 mmol, 1.5 equiv) in dioxane (3.0 mL) and H₂O (0.5 mL) were added Pd(dppf)Cl₂ (116.0 mg, 0.127 mmol, 0.1 equiv) and K₃PO₄ (1.24 g, 3.802 mmol, 3.0 equiv). After stirring for 2 h at 70° C. under a nitrogen atmosphere, the resulting mixture was concentrated under reduced pressure. The residue was purified by silica gel column chromatography and eluted with PE/EA (1:1) to afford 6-bromo-2-{8-fluoro-[1,2,4]triazolo[4,3-a]pyridin-6-yl}quinazoline (D307, 350.0 mg). LCMS: (ES, m/z): 344 [M+H]⁺.

### Synthesis of Intermediate D308

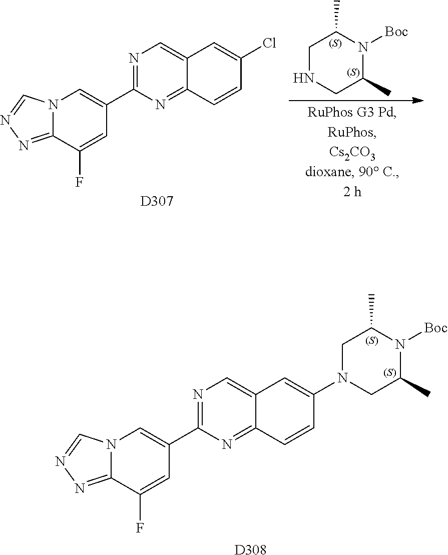

To a solution of Intermediate D307 (350.0 mg, 1.017 mmol, 1.0 equiv) and tert-butyl (2S,6S)-2,6-dimethylpiperazine-1-carboxylate (261.5 mg, 1.220 mmol, 1.2 equiv) in dioxane (4.0 mL) were added RuPhos Palladacycle Gen.3 (85.0 mg, 0.102 mmol, 0.1 equiv), RuPhos (94.9 mg, 0.203 mmol, 0.2 equiv) and Cs₂CO₃ (994.0 mg, 3.051 mmol, 3.0 equiv). After stirring for 2 h at 90° C. under a nitrogen atmosphere, the resulting mixture was concentrated under reduced pressure. The residue was purified by silica gel column chromatography and eluted with PE/EA (1:1) to afford tert-butyl (2S,6S)-4-(2-{8-fluoro-[1,2,4]triazolo[4,3-a]pyridin-6-yl}quinazolin-6-yl)-2,6-dimethylpiperazine-1-carboxylate (D308, 200.0 mg). LCMS: (ES, m/z): 478 [M+H]⁺.

### Synthesis of Compound 820

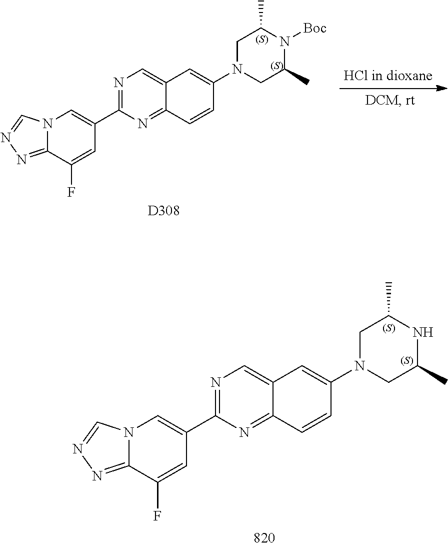

To a stirred solution of Intermediate D308 (200.0 mg, 0.419 mmol, 1.0 equiv) in DCM (3.0 mL) was added HCl(gas) in 1,4-dioxane (0.20 mL, 6.587 mmol, 15.72 equiv) dropwise at 0° C., stirred for 1 h at room temperature, and then concentrated under reduced pressure. The crude product was purified by Prep-HPLC following Condition 19, Gradient 1 to afford 6-[(3S,5S)-3,5-dimethylpiperazin-1-yl]-2-{8-fluoro-[1,2,4]triazolo[4,3-a]pyridin-6-yl}quinazoline (Compound 820, 128.7 mg). Absolute stereochemistry of the compound was arbitrarily assigned. LCMS: (ES, m/z): 378 [M+H]⁺. ¹H NMR: (400 MHz, DMSO-d₆) δ 9.56-9.50 (m, 2H), 9.47 (s, 1H), 8.14 (d, J=12.2 Hz, 1H), 7.90 (d, J=1.5 Hz, 2H), 7.35 (d, J=1.7 Hz, 1H), 3.38 (dd, J=11.8, 3.4 Hz, 2H), 3.24 (d, J=6.4, 3.3 Hz, 2H), 3.03 (dd, J=11.8, 6.3 Hz, 2H), 2.20 (s, 1H), 1.13 (d, J=6.4 Hz, 6H).

### Example 326: Synthesis of Compound 822

### Synthesis of Intermediate D310

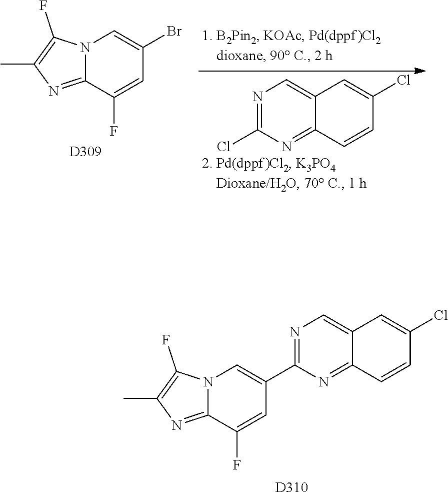

To a solution of 6-bromo-3,8-difluoro-2-methylimidazo[1,2-a]pyridine (D309, 100 mg, 0.405 mmol, 1 equiv) and 4,4,5,5-tetramethyl-2-(4,4,5,5-tetramethyl-1,3,2-dioxaborolan-2-yl)-1,3,2-dioxaborolane (123 mg, 0.486 mmol, 1.2 equiv) in dioxane (4 mL) were added KOAc (119 mg, 1.215 mmol, 3.0 equiv) and Pd(dppf)Cl₂ (33 mg, 0.041 mmol, 0.1 equiv). After stirring for 2 h at 90° C. under a nitrogen atmosphere, the mixture was allowed to cool to room temperature. To the above mixture were added 2,6-dichloroquinazoline (100 mg, 0.502 mmol, 1.24 equiv), Pd(dppf)Cl₂ (33 mg, 0.041 mmol, 0.1 equiv), K₃PO₄ (257 mg, 1.215 mmol, 3.0 equiv) and H₂O (1 mL). The resulting mixture was stirred for 1 h at 70° C. under a nitrogen atmosphere and then concentrated under reduced pressure. The residue was purified by silica gel column chromatography and eluted with PE:EA (1:1) to afford 6-chloro-2-{3,8-difluoro-2-methylimidazo[1,2-a]pyridin-6-yl}quinazoline (D310, 80 mg). LCMS: (ES, m/z): 331[M+H]⁺.

### Synthesis of Intermediate D311

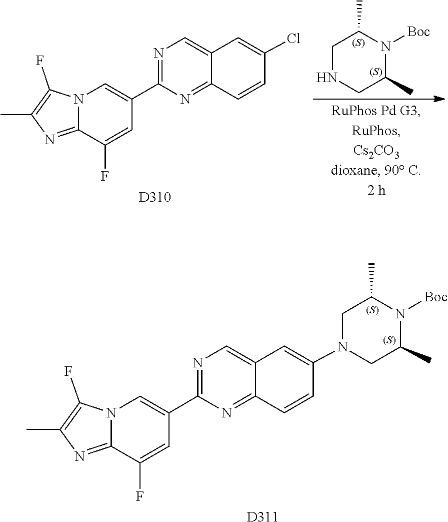

To a solution of Intermediate D310 (70 mg, 0.212 mmol, 1 equiv) and tert-butyl (2S,6S)-2,6-dimethylpiperazine-1-carboxylate (54.4 mg, 0.254 mmol, 1.2 equiv) in dioxane (2 mL) were added Cs₂CO₃ (206.8 mg, 0.636 mmol, 3.0 equiv), RuPhos (19.7 mg, 0.042 mmol, 0.2 equiv) and RuPhos Palladacycle Gen.3 (17.7 mg, 0.021 mmol, 0.1 equiv). After stirring for 2 h at 90° C. under a nitrogen atmosphere, the resulting mixture was concentrated under reduced pressure. The residue was purified by reversed-phase flash chromatography following Condition 10, Gradient 8 as previously described to afford tert-butyl (2S,6S)-4-(2-{3,8-difluoro-2-methylimidazo[1,2-a]pyridin-6-yl}quinazolin-6-yl)-2,6-dimethylpiperazine-1-carboxylate (D311, 70 mg). LCMS: (ES, m/z): 509[M+H]⁺.

### Synthesis of Compound 822

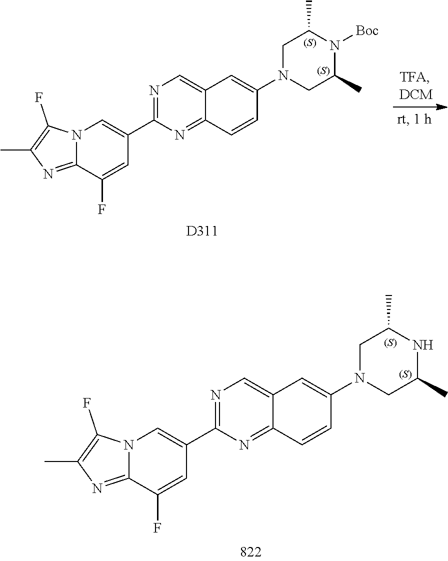

TFA (0.2 mL) was added dropwise at room temperature to solution of Intermediate D311 (60 mg, 0.118 mmol, 1 equiv) in DCM (2 mL). The resulting mixture was stirred for 1 h and then concentrated under reduced pressure. The residue was purified by reversed-phase flash chromatography following Condition 10, Gradient 6 as previously described to afford 2-{3,8-difluoro-2-methylimidazo[1,2-a]pyridin-6-yl}-6-[(3S,5S)-3,5-dimethylpiperazin-1-yl]quinazoline (Compound 822, 20 mg). Absolute stereochemistry of the compound was arbitrarily assigned. LCMS: (ES, m/z): 409[M+H]⁺. ¹H NMR: (300 MHz, DMSO-d₆) δ 9.44 (s, 1H), 8.92 (d, J=1.8 Hz, 1H), 7.99 (d, J=12.8 Hz, 1H), 7.89 (s, 2H), 7.33 (s, 1H), 3.42-3.32 (m, 2H), 3.24 (td, J=6.4, 3.3 Hz, 2H), 3.01 (dd, J=11.7, 6.2 Hz, 2H), 2.38 (d, J=1.5 Hz, 3H), 1.13 (d, J=6.4 Hz, 6H).

### Example 327: Synthesis of Compound 825

### Synthesis of Intermediate D312

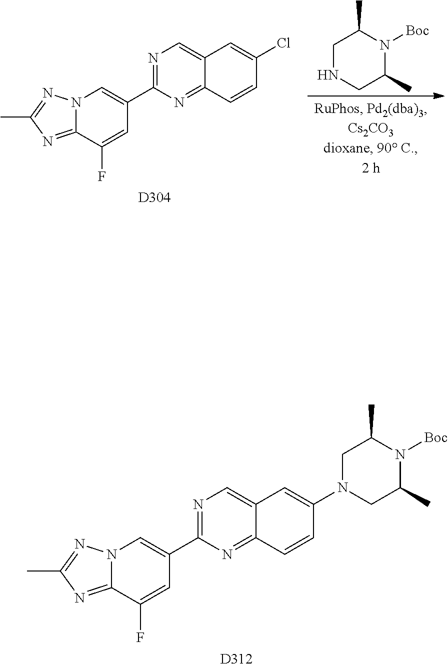 [SMILES: Cc1nc2c(F)cc(-c3ncc4cc(Cl)ccc4n3)cn2n1 ; C[C@@H]1CNC[C@H](C)N1C(=O)OC(C)(C)C ; "D304" ; Cc1nc2c(F)cc(-c3ncc4cc(N5C[C@@H](C)N(C(=O)OC(C)(C)C)[C@@H](C)C5)ccc4n3)cn2n1 ; "D312"]

To a solution of 6-chloro-2-{8-fluoro-2-methyl-[1,2,4]triazolo[1,5-a]pyridin-6-yl}quinazoline (D304, 300.0 mg, 0.956 mmol, 1.0 equiv) and tert-butyl (2R,6S)-2,6-dimethylpiperazine-1-carboxylate (245.92 mg, 1.147 mmol, 1.2 equiv) in dioxane (3.0 mL) were added Pd₂(dba)₃ (87.57 mg, 0.096 mmol, 0.1 equiv), RuPhos (89.25 mg, 0.191 mmol, 0.2 equiv) and Cs₂CO₃ (934.71 mg, 2.868 mmol, 3.0 equiv). After stirring for 2 h at 90° C. under a nitrogen atmosphere, the resulting mixture was concentrated under reduced pressure. The residue was purified by silica gel column chromatography and eluted with PE/EA (1:1) to afford tert-butyl (2R,6S)-4-(2-{8-fluoro-2-methyl-[1,2,4]triazolo[1,5-a]pyridin-6-yl}quinazolin-6-yl)-2,6-dimethylpiperazine-1-carboxylate (D312, 200.0 mg). LCMS: (ES, m/z): 492 [M+H]⁺.

### Synthesis of Compound 825

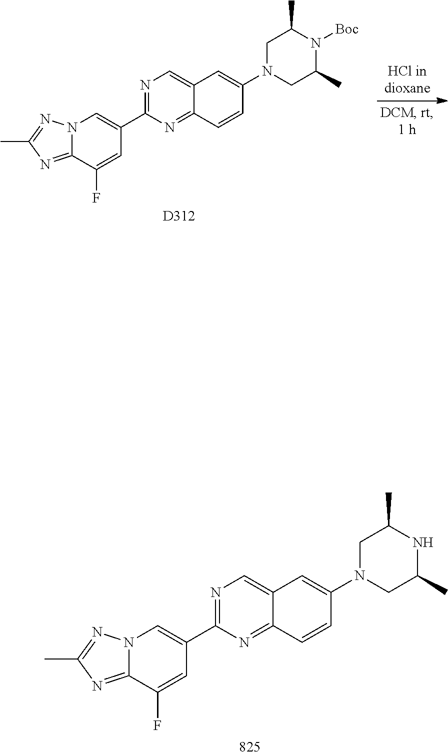

To a stirred solution of Intermediate D312 (200.0 mg, 0.407 mmol, 1 equiv) in DCM (3.0 mL) was added HCl(gas) in 1,4-dioxane (0.2 mL, 6.583 mmol, 16.18 equiv) dropwise at 0° C. The resulting mixture was stirred for 1 h at room temperature and subsequently concentrated under reduced pressure. The crude product was purified by Prep-HPLC following Condition 19, Gradient 1 as previously described to afford 6-[(3R,5S)-3,5-dimethylpiperazin-1-yl]-2-{8-fluoro-2-methyl-[1,2,4]triazolo[1,5-a]pyridin-6-yl}quinazoline (Compound 825, 65.6 mg). Absolute stereochemistry of the compound was arbitrarily assigned. LCMS: (ES, m/z): 392 [M+H]⁺. ¹H NMR: (400 MHz, DMSO-d₆) δ 9.52-9.46 (m, 2H), 8.40 (dd, J=11.5, 1.3 Hz, 1H), 7.99-7.88 (m, 2H), 7.36 (d, J=2.5 Hz, 1H), 3.82 (d, J=11.2 Hz, 2H), 2.90 (s, 2H), 2.56 (s, 3H), 2.34 (d, J=11.0 Hz, 2H), 1.08 (d, J=6.3 Hz, 6H).

### Example 328: Synthesis of Compound 826

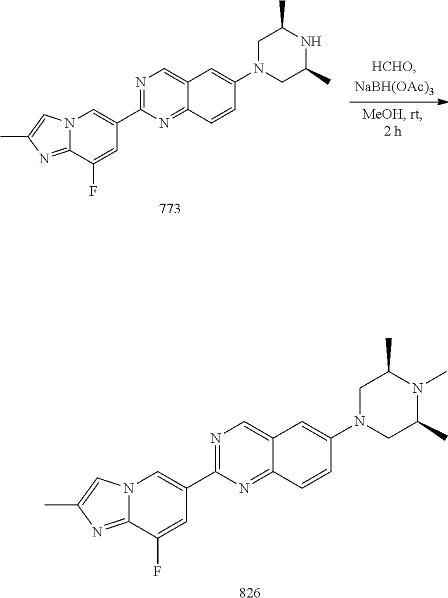

To a stirred solution of 6-[(3R,5S)-3,5-dimethylpiperazin-1-yl]-2-{8-fluoro-2-methylimidazo[1,2-a]pyridin-6-yl}quinazoline (Compound 773, 150.0 mg, 0.384 mmol, 1.0 equiv) and HCHO (93.5 mg, 1.152 mmol, 3.0 equiv, 37%) in MeOH (2.0 mL) was added NaBH(OAc)₃ (407.1 mg, 1.920 mmol, 5.0 equiv) in portions at room temperature. The resulting mixture was stirred for 2 h at room temperature and subsequently concentrated under reduced pressure. The residue was purified by silica gel column chromatography and eluted with CH₂Cl₂/MeOH (10:1) to afford 2-{8-fluoro-2-methylimidazo[1,2-a]pyridin-6-yl}-6-[(3R,5S)-3,4,5-trimethylpiperazin-1-yl]quinazoline (Compound 826, 95.0 mg). Absolute stereochemistry of the compound was arbitrarily assigned. LCMS: (ES, m/z): 405 [M+H]⁺. ¹H NMR: (400 MHz, DMSO-d₆) δ 9.48-9.42 (m, 2H), 8.05-8.00 (m, 1H), 8.00-7.92 (m, 2H), 7.89 (d, J=9.3 Hz, 1H), 7.37 (d, J=2.6 Hz, 1H), 3.82 (d, J=11.9 Hz, 2H), 2.58 (t, J=11.4 Hz, 2H), 2.42-2.37 (m, 3H), 2.31 (d, J=9.2 Hz, 2H), 2.22 (s, 3H), 1.13 (d, J=6.1 Hz, 6H).

### Example 329: Synthesis of Compound 828

### Synthesis of Intermediate D314

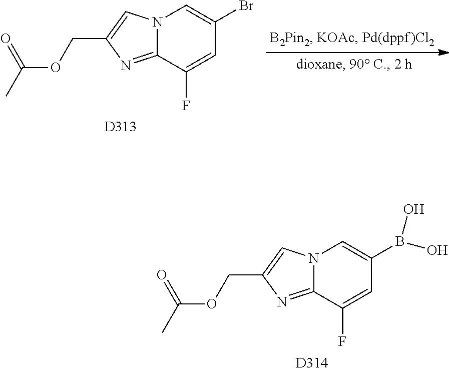

To a solution of {6-bromo-8-fluoroimidazo[1,2-a]pyridin-2-yl}methyl acetate (D313, 320 mg, 1.115 mmol, 1 equiv) in 1,4-dioxane (5 mL) were added B₂Pin₂ (311 mg, 1.227 mmol, 1.1 equiv), KOAc (328 mg, 3.345 mmol, 3 equiv) and Pd(dppf)Cl₂·CH₂Cl₂ (91 mg, 0.112 mmol, 0.1 equiv). The mixture was stirred for 2 h at 90° C. under nitrogen atmosphere and then concentrated under reduced pressure to afford 2-[(acetyloxy)methyl]-8-fluoroimidazo[1,2-a]pyridin-6-ylboronic acid (D314, 260 mg). LCMS: (ES, m/z):253 [M+H]⁺.

### Synthesis of Intermediate D315

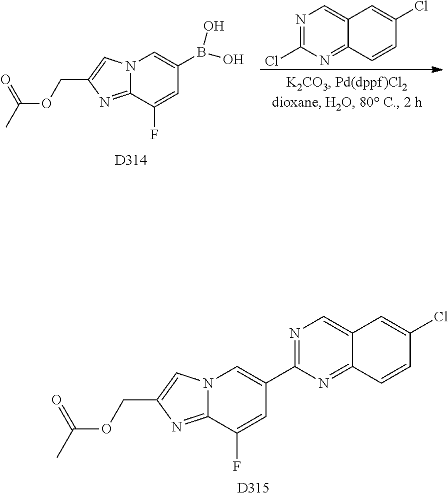

To a solution of Intermediate D314 (260 mg, 1.032 mmol, 1 equiv) in 1,4-dioxane (4 mL)/H₂O (0.4 mL) were added 2,6-dichloroquinazoline (205 mg, 1.032 mmol, 1 equiv), K₂CO₃ (428 mg, 3.096 mmol, 3 equiv) and Pd(dppf)Cl₂·CH₂Cl₂ (84 mg, 0.103 mmol, 0.1 equiv). The mixture was stirred for 2 h at 80° C. under nitrogen atmosphere, then diluted with water (20 mL), and extracted with DCM (2×20 mL). The combined organic layers were washed with brine (2×30 mL), dried over anhydrous Na₂SO₄, and the filtrate was concentrated under reduced pressure. The residue was purified by silica gel column chromatography and eluted with DCM/EA (30:1) to afford [6-(6-chloroquinazolin-2-yl)-8-fluoroimidazo[1,2-a]pyridin-2-yl]methyl acetate (D315, 210 mg). LCMS: (ES, m/z):371 [M+H]⁺.

### Synthesis of Intermediate D316

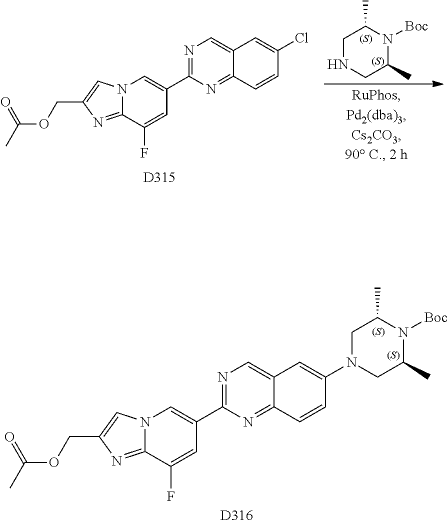

To a solution of Intermediate D315 (100 mg, 0.270 mmol, 1 equiv) in 1,4-dioxane (2 mL) were added tert-butyl (2S,6S)-2,6-dimethylpiperazine-1-carboxylate (64 mg, 0.297 mmol, 1.1 equiv), Cs₂CO₃ (176 mg, 0.540 mmol, 2 equiv), RuPhos (25 mg, 0.054 mmol, 0.2 equiv) and RuPhos Pd Gen.3 (13 mg, 0.027 mmol, 0.1 equiv). The mixture was stirred for 2 h at 90° C. under nitrogen atmosphere and subsequently concentrated under reduced pressure. The residue was purified by silica gel column chromatography and eluted with PE/EA (3:2) to afford tert-butyl (2S,6S)-4-(2-{2-[(acetyloxy)methyl]-8-fluoroimidazo[1,2-a]pyridin-6-yl}quinazolin-6-yl)-2,6-dimethylpiperazine-1-carboxylate (D316, 75 mg). LCMS: (ES, m/z):549 [M+H]⁺.

### Synthesis of Intermediate D317

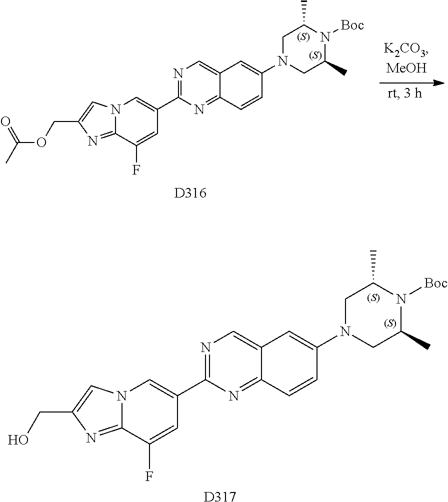

To a solution of Intermediate D316 (75 mg, 0.137 mmol, 1 equiv) in MeOH (1.5 mL) was added K₂CO₃ (57 mg, 0.411 mmol, 3 equiv), The mixture was stirred for 3 h at room temperature, concentrated under reduced pressure and diluted with DCM (10 mL). The resulting mixture was filtered, washed with DCM (2×10 mL), and finally concentrated under reduced pressure to afford tert-butyl (2S,6S)-4-{2-[8-fluoro-2-(hydroxymethyl)imidazo[1,2-a]pyridin-6-yl]quinazolin-6-yl}-2,6-dimethylpiperazine-1-carboxylate (D317, 69 mg). LCMS: (ES, m/z):507 [M+H]⁺.

### Synthesis of Compound 828

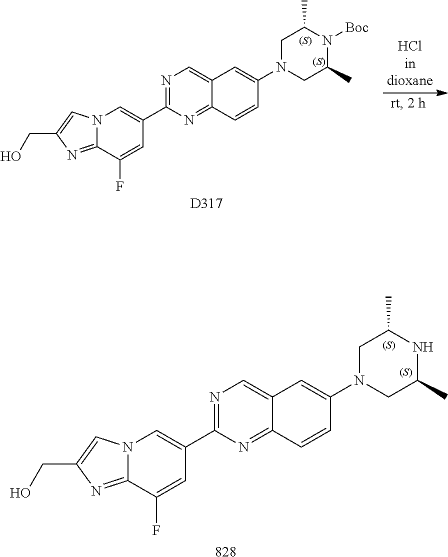

To a solution of Intermediate D317 (70 mg, 0.138 mmol, 1 equiv) in DCM (1 mL) was added HCl(gas) in 1,4-dioxane (0.3 mL). The mixture was stirred for 2 h at room temperature and subsequently concentrated under reduced pressure. The crude product was purified by Prep-HPLC following Condition 6, Gradient 9 to afford (6-{6-[(3S,5S)-3,5-dimethylpiperazin-1-yl]quinazolin-2-yl}-8-fluoroimidazo[1,2-a]pyridin-2-yl)methanol (Compound 828, 18 mg). Absolute stereochemistry of the compound was arbitrarily assigned. LCMS: (ES, m/z):407 [M+H]⁺. ¹H NMR: (400 MHz, DMSO-d₆) δ 9.51 (d, J=1.3 Hz, 1H), 9.45 (s, 1H), 8.15 (d, J=3.1 Hz, 1H), 8.00 (dd, J=12.5, 1.4 Hz, 1H), 7.88 (d, J=1.5 Hz, 2H), 7.33 (t, J=1.6 Hz, 1H), 5.33 (t, J=5.7 Hz, 1H), 4.65 (d, J=5.4 Hz, 2H), 3.36 (dd, J=11.7, 3.4 Hz, 2H), 3.24 (td, J=6.4, 3.4 Hz, 2H), 3.01 (dd, J=11.7, 6.2 Hz, 2H), 2.18-2.17 (m, 1H), 1.13 (d, J=6.5 Hz, 6H).

### Example 330: Synthesis of Compound 829

### Synthesis of Intermediate D319

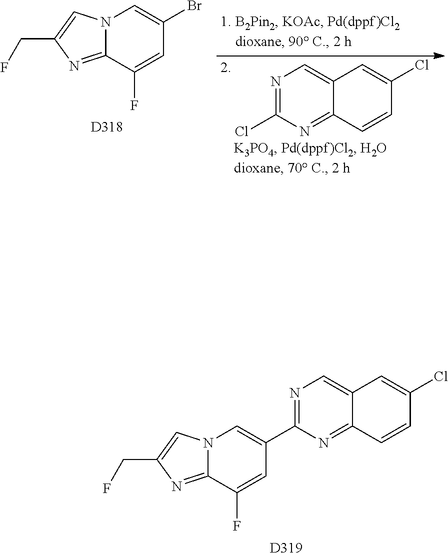

To a solution of 6-bromo-8-fluoro-2-(fluoromethyl)imidazo[1,2-a]pyridine (D318, 200 mg, 0.810 mmol, 1 equiv) and 4,4,5,5-tetramethyl-2-(4,4,5,5-tetramethyl-1,3,2-dioxaborolan-2-yl)-1,3,2-dioxaborolane (247 mg, 0.972 mmol, 1.2 equiv) in dioxane (4 mL) were added KOAc (238 mg, 2.430 mmol, 3.0 equiv) and Pd(dppf)Cl₂ (66 mg, 0.081 mmol, 0.1 equiv). After stirring for 2 h at 90° C. under a nitrogen atmosphere, the resulting mixture was allowed to cool to room temperature. Then 2,6-dichloroquinazoline (200 mg, 1.005 mmol, 1.24 equiv), Pd(dppf)Cl₂ (66 mg, 0.081 mmol, 0.1 equiv), K₃PO₄ (516 mg, 2.430 mmol, 3.0 equiv) and H₂O (1 mL) were added, and the resulting mixture was stirred for an additional 2 h at 70° C. under nitrogen atmosphere and subsequently concentrated under reduced pressure. The residue was purified by silica gel column chromatography and eluted with PE:EA (1:1) to afford 6-chloro-2-[8-fluoro-2-(fluoromethyl)imidazo[1,2-a]pyridin-6-yl]quinazoline (D319, 100 mg). LCMS: (ES, m/z): 331[M+H]⁺.

### Synthesis of Intermediate D320

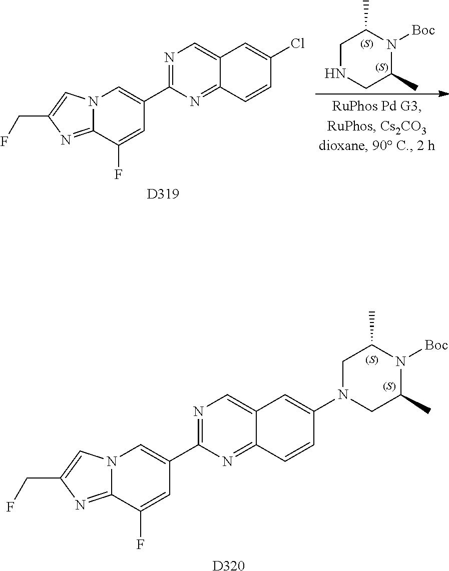

To a solution of Intermediate D319 (100 mg, 0.302 mmol, 1 equiv) and tert-butyl (2S,6S)-2,6-dimethylpiperazine-1-carboxylate (78 mg, 0.362 mmol, 1.2 equiv) in dioxane (3 mL) were added Cs₂CO₃ (296 mg, 0.906 mmol, 3.0 equiv), RuPhos (28 mg, 0.060 mmol, 0.2 equiv) and RuPhos Palladacycle Gen.3 (25 mg, 0.030 mmol, 0.1 equiv). After stirring for 2 h at 90° C. under a nitrogen atmosphere, the resulting mixture was concentrated under reduced pressure. The residue was purified by reversed-phase flash chromatography following Condition 10, Gradient 8 to afford tert-butyl (2S,6S)-4-{2-[8-fluoro-2-(fluoromethyl)imidazo[1,2-a]pyridin-6-yl]quinazolin-6-yl}-2,6-dimethylpiperazine-1-carboxylate (D320, 76 mg). LCMS: (ES, m/z):509 [M+H]⁺.

### Synthesis of Compound 829

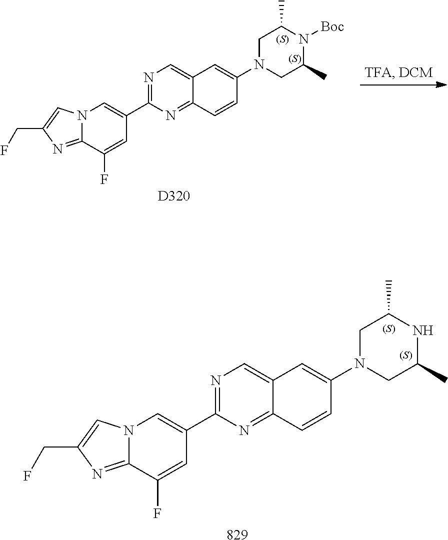

To a stirred solution of Intermediate D320 (70 mg, 0.138 mmol, 1 equiv) in DCM (2 mL) was added TFA (0.2 mL) dropwise at room temperature. The resulting mixture was stirred for 1 h at room temperature, neutralized to pH 8 with NH₃ in MeOH, and then concentrated under reduced pressure. The residue was purified by reversed-phase flash chromatography following Condition 10, Gradient 15 as previously described to afford 6-[(3S,5S)-3,5-dimethylpiperazin-1-yl]-2-[8-fluoro-2-(fluoromethyl)imidazo[1,2-a]pyridin-6-yl]quinazoline (Compound 829, 20 mg). Absolute stereochemistry of the compound was arbitrarily assigned. LCMS: (ES, m/z):409 [M+H]⁺. ¹H NMR: (300 MHz, DMSO-d₆) δ 9.56 (d, J=1.3 Hz, 1H), 9.46 (s, 1H), 8.42 (d, J=3.2 Hz, 1H), 8.08 (d, J=12.7 Hz, 1H), 7.90 (s, 2H), 7.35 (s, 1H), 5.64 (s, 1H), 5.48 (s, 1H), 3.37 (d, J=12.0 Hz, 2H), 3.24 (s, 2H), 3.02 (dd, J=11.8, 6.2 Hz, 2H), 1.13 (d, J=6.5 Hz, 6H).

### Example 331: Synthesis of Compound 834

### Synthesis of Intermediate D321

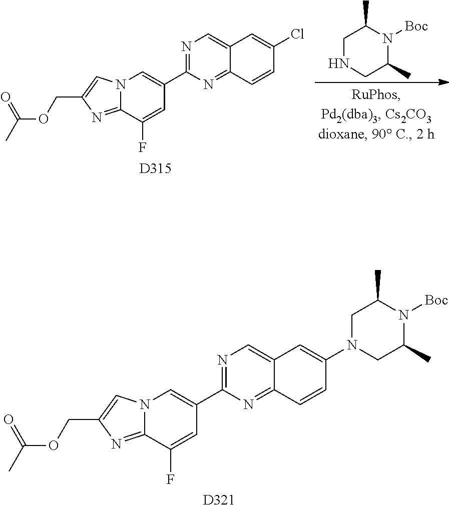

To a solution of [6-(6-chloroquinazolin-2-yl)-8-fluoroimidazo[1,2-a]pyridin-2-yl]methyl acetate (D315, 110 mg, 0.297 mmol, 1 equiv) in 1,4-dioxane (2 mL) were added tert-butyl (2R,6S)-2,6-dimethylpiperazine-1-carboxylate (70 mg, 0.327 mmol, 1.1 equiv), Cs₂CO₃ (193 mg, 0.594 mmol, 2 equiv), RuPhos (28 mg, 0.059 mmol, 0.2 equiv) and RuPhos Pd Gen.3 (25 mg, 0.030 mmol, 0.1 equiv). The mixture was stirred for 2 h at 90° C. under nitrogen atmosphere and then concentrated under reduced pressure. The residue was purified by silica gel column chromatography and eluted with PE/EA (3:2) to afford tert-butyl (2R,6S)-4-(2-{2-[(acetyloxy)methyl]-8-fluoroimidazo[1,2-a]pyridin-6-yl}quinazolin-6-yl)-2,6-dimethylpiperazine-1-carboxylate (D321, 95 mg). LCMS: (ES, m/z):549 [M+H]⁺.

### Synthesis of Intermediate D322

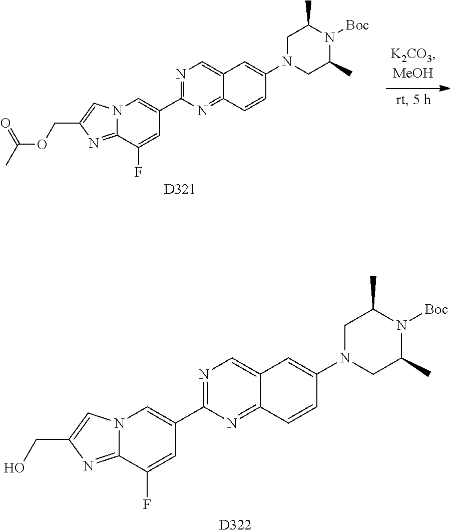 [SMILES: CC(=O)OCc1cn2cc(-c3ncc4cc(N5C[C@@H](C)N(C(=O)OC(C)(C)C)[C@@H](C)C5)ccc4n3)cc(F)c2n1 ; "D321" ; C[C@@H]1CN(c2ccc3nc(-c4cc(F)c5nc(CO)cn5c4)ncc3c2)C[C@H](C)N1C(=O)OC(C)(C)C ; "D322"]

To a solution of Intermediate D321 (95 mg, 0.173 mmol, 1 equiv) in MeOH (1.5 mL) was added K₂CO₃ (72 mg, 0.519 mmol, 3 equiv). The mixture was stirred for 5 h at room temperature, concentrated under reduced pressure and then diluted with DCM (10 mL). The resulting mixture was filtered, washed with DCM (2×5 mL), and the filtrate was concentrated under reduced pressure to afford tert-butyl (2R,6S)-4-{2-[8-fluoro-2-(hydroxymethyl)imidazo[1,2-a]pyridin-6-yl]quinazolin-6-yl}-2,6-dimethylpiperazine-1-carboxylate (D322, 87 mg). LCMS: (ES, m/z):507 [M+H]⁺.

### Synthesis of Compound 834

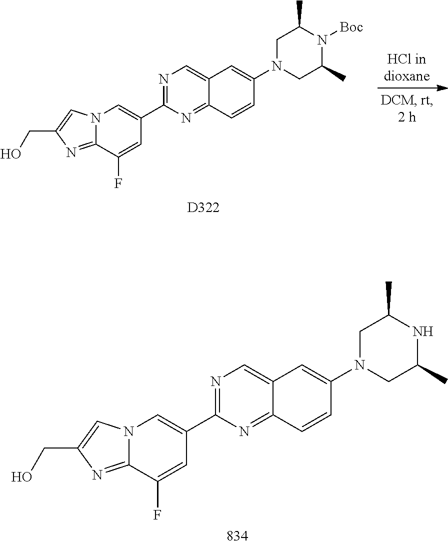 [SMILES: C[C@@H]1CN(c2ccc3nc(-c4cc(F)c5nc(CO)cn5c4)ncc3c2)C[C@H](C)N1C(=O)OC(C)(C)C ; "D322" ; C[C@@H]1CN(c2ccc3nc(-c4cc(F)c5nc(CO)cn5c4)ncc3c2)C[C@H](C)N1 ; "834"]

To a solution of Intermediate D322 (87 mg, 0.171 mmol, 1 equiv) in DCM (1.5 mL) was added HCl(gas) in 1,4-dioxane (0.4 mL, 4M). The mixture was stirred for 2 h at room temperature and subsequently concentrated under reduced pressure. The crude product was purified by Prep-HPLC following Condition 6, Gradient 8 as previously described to afford (6-{6-[(3R,5S)-3,5-dimethylpiperazin-1-yl]quinazolin-2-yl}-8-fluoroimidazo[1,2-a]pyridin-2-yl)methanol (Compound 834, 25 mg). Absolute stereochemistry of the compound was arbitrarily assigned. LCMS: (ES, m/z):407 [M+H]⁺. ¹H NMR: (400 MHz, DMSO-d₆) δ 9.51 (d, J=1.3 Hz, 1H), 9.46 (s, 1H), 8.15 (d, J=3.1 Hz, 1H), 7.99 (dd, J=12.5, 1.4 Hz, 1H), 7.95-7.85 (m, 2H), 7.35 (d, J=2.6 Hz, 1H), 5.33 (t, J=5.7 Hz, 1H), 4.65 (d, J=5.2 Hz, 2H), 3.80 (dd, J=11.9, 2.7 Hz, 2H), 2.89 (ddt, J=11.4, 7.9, 3.9 Hz, 2H), 2.35-2.27 (m, 2H), 1.08 (d, J=6.3 Hz, 6H).

### Example 332: Synthesis of Compound 835

### Synthesis of Intermediate D323

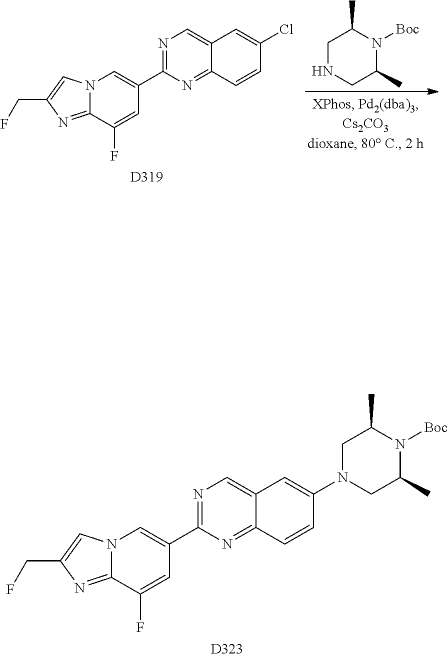

To a solution of 6-chloro-2-[8-fluoro-2-(fluoromethyl)imidazo[1,2-a]pyridin-6-yl]quinazoline (D319, 80 mg, 0.242 mmol, 1 equiv) and tert-butyl (2R,6S)-2,6-dimethylpiperazine-1-carboxylate (104 mg, 0.484 mmol, 2.0 equiv) in dioxane (2 mL) were added Cs₂CO₃ (236 mg, 0.726 mmol, 3.0 equiv), XPhos (23 mg, 0.048 mmol, 0.2 equiv) and Pd₂(dba)₃ (25 mg, 0.024 mmol, 0.1 equiv). After stirring for 2 h at 80° C. under a nitrogen atmosphere, the resulting mixture was concentrated under reduced pressure. The residue was purified by reversed-phase flash chromatography following Condition 10, Gradient 8 as previously described to afford tert-butyl (2R,6S)-4-{2-[8-fluoro-2-(fluoromethyl)imidazo[1,2-a]pyridin-6-yl]quinazolin-6-yl}-2,6-dimethylpiperazine-1-carboxylate (D323, 120 mg). LCMS: (ES, m/z): 509[M+H]⁺.

### Synthesis of Compound 835

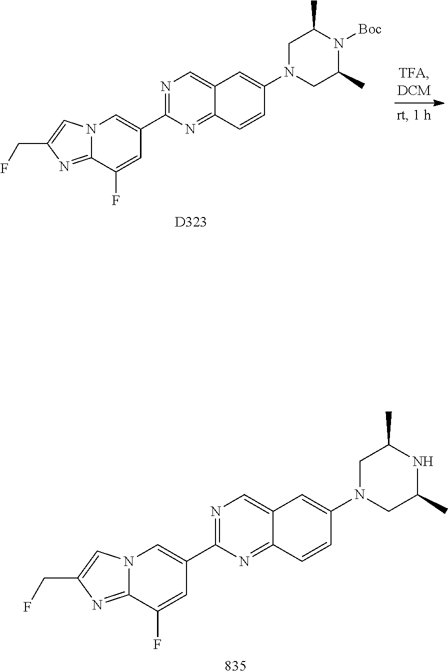 [SMILES: C[C@@H]1CN(c2ccc3nc(-c4cc(F)c5nc(CF)cn5c4)ncc3c2)C[C@H](C)N1C(=O)OC(C)(C)C ; "D323" ; C[C@@H]1CN(c2ccc3nc(-c4cc(F)c5nc(CF)cn5c4)ncc3c2)C[C@H](C)N1 ; "835"]

To a stirred solution of Intermediate D323 (120 mg, 0.197 mmol, 1 equiv) in DCM (4 mL) was added TFA (0.4 mL) dropwise at room temperature. The resulting mixture was stirred for 1 h and then concentrated under reduced pressure. The residue was purified by reversed-phase flash chromatography following Condition 10, Gradient 8 as previously described to afford 6-[(3R,5S)-3,5-dimethylpiperazin-1-yl]-2-[8-fluoro-2-(fluoromethyl)imidazo[1,2-a]pyridin-6-yl]quinazoline (Compound 835, 60 mg). Absolute stereochemistry of the compound was arbitrarily assigned. LCMS: (ES, m/z):409 [M+H]⁺. ¹H NMR: (300 MHz, DMSO-d₆) δ 9.55 (d, J=1.3 Hz, 1H), 9.47 (s, 1H), 8.42 (t, J=3.2 Hz, 1H), 8.07 (dd, J=12.4, 1.3 Hz, 1H), 7.99-7.85 (m, 2H), 7.36 (d, J=2.4 Hz, 1H), 5.63 (s, 1H), 5.47 (s, 1H), 3.82 (d, J=11.4 Hz, 2H), 2.92 (s, 2H), 2.33 (t, J=11.1 Hz, 2H), 1.09 (d, J=6.2 Hz, 6H).

### Example 333: Synthesis of Compound 837

### Synthesis of Intermediate D324

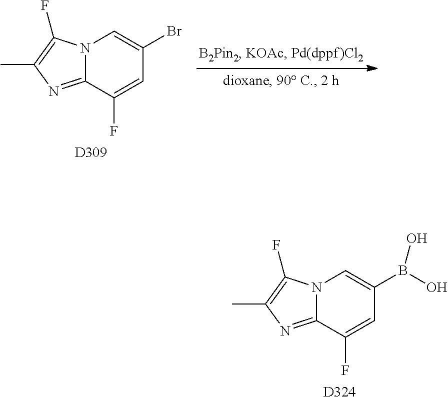

To a solution of 6-bromo-3,8-difluoro-2-methylimidazo[1,2-a]pyridine (D309, 180 mg, 0.729 mmol, 1 equiv) in 1,4-dioxane (2.5 mL) were added B₂Pin₂ (241 mg, 0.948 mmol, 1.3 equiv), KOAc (215 mg, 2.187 mmol, 3 equiv) and Pd(dppf)Cl₂·CH₂Cl₂ (59 mg, 0.073 mmol, 0.1 equiv). The mixture was stirred for 2 h at 90° C. under nitrogen atmosphere, concentrated under reduced pressure, and used directly in the next step without further purification. LCMS: (ES, m/z):213 [M+H]⁺.

### Synthesis of Intermediate D325

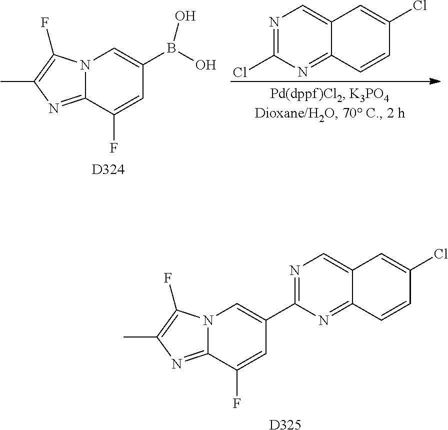

To a solution of 3,8-difluoro-2-methylimidazo[1,2-a]pyridin-6-ylboronic acid (D324, crude from previous step) in 1,4-dioxane (2.5 mL)/H₂O (0.25 mL) were added 2,6-dichloroquinazoline (132 mg, 0.661 mmol, 1 equiv), K₃PO₄ (421 mg, 1.983 mmol, 3 equiv) and Pd(dppf)Cl₂·CH₂Cl₂ (54 mg, 0.066 mmol, 0.1 equiv). The mixture was stirred for 2 h at 70° C. under nitrogen atmosphere, diluted with water (15 mL), and then extracted with DCM (2×20 mL). The combined organic layers were washed with brine (2×30 mL), dried over anhydrous Na₂SO₄, and the filtrate was concentrated under reduced pressure. The residue was purified by silica gel column chromatography and eluted with DCM/EA (20:1) to afford 6-chloro-2-{3,8-difluoro-2-methylimidazo[1,2-a]pyridin-6-yl}quinazoline (D325, 115 mg). LCMS: (ES, m/z):331 [M+H]⁺.

### Synthesis of Intermediate D326

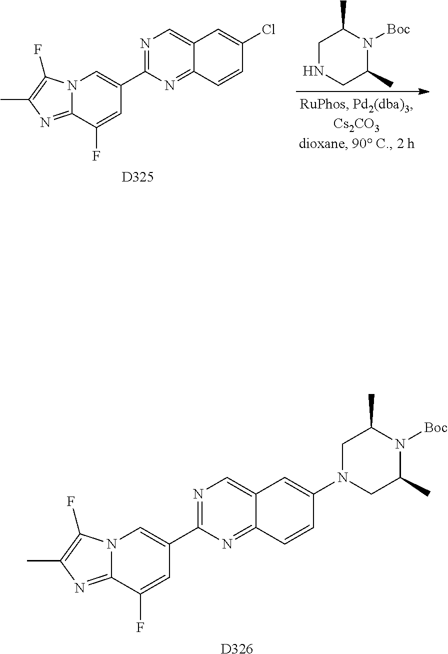

To a solution of Intermediate D325 (115 mg, 0.348 mmol, 1 equiv) in 1,4-dioxane (2 mL) were added tert-butyl (2R,6S)-2,6-dimethylpiperazine-1-carboxylate (89 mg, 0.418 mmol, 1.2 equiv), Cs₂CO₃ (227 mg, 0.696 mmol, 2 equiv), RuPhos (32 mg, 0.070 mmol, 0.2 equiv) and Pd₂(dba)₃ (32 mg, 0.035 mmol, 0.1 equiv). The mixture was stirred for 2 h at 90° C. under nitrogen atmosphere and then concentrated under reduced pressure. The residue was purified by silica gel column chromatography and eluted with DCM/MeOH (20:1) to afford tert-butyl (2R,6S)-4-(2-{3,8-difluoro-2-methylimidazo[1,2-a]pyridin-6-yl}quinazolin-6-yl)-2,6-dimethylpiperazine-1-carboxylate (D326, 95 mg). LCMS: (ES, m/z):509 [M+H]⁺.

### Synthesis of Compound 837

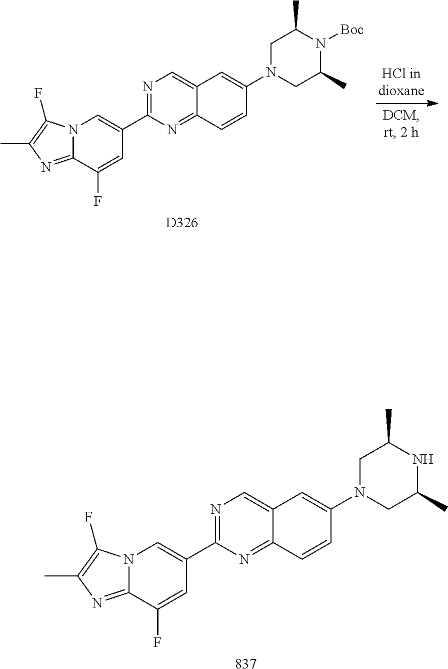

To a solution of Intermediate D326 (95 mg, 0.187 mmol, 1 equiv) in DCM (1 mL) was added HCl(gas) in 1,4-dioxane (0.5 mL). The mixture was stirred for 2 h at room temperature, concentrated under reduced pressure, and adjusted to pH 8 with saturated NaHCO₃ (aq.). The precipitated solids were collected by filtration and washed with water (2×10 mL). The residue was purified by trituration with hexane, and the resulting solid was dried by air to afford 2-{3,8-difluoro-2-methylimidazo[1,2-a]pyridin-6-yl}-6-[(3R,5S)-3,5-dimethylpiperazin-1-yl]quinazoline (Compound 837, 50 mg). Absolute stereochemistry of the compound was arbitrarily assigned. LCMS: (ES, m/z):409 [M+H]⁺. ¹H NMR: (400 MHz, DMSO-d₆) δ 9.46 (s, 1H), 8.94 (d, J=1.3 Hz, 1H), 8.03-7.87 (m, 3H), 7.36 (d, J=2.4 Hz, 1H), 3.83 (d, J=11.5 Hz, 2H), 2.93-2.92 (m, 2H), 2.43-2.27 (m, 5H), 1.10 (d, J=6.2 Hz, 6H).

### Example 332: Exemplary Splicing Assay for Monitoring Expression Levels of Splice Variants

Compounds described herein were used to modulate RNA transcript abundance in cells. The expression of a target mRNA was measured by detecting the formation of an exon-exon junction in the canonical transcript (CJ). A compound mediated exon-inclusion event was detected by observing an increase in formation of a new junction with an alternative exon (AJ). Real-time qPCR assays were used to detect these splicing switches and interrogate the potency of various compounds towards different target genes. A high-throughput real time quantitative PCR (RT-qPCR) assay was developed to measure these two isoforms of the mRNA (CJ and AJ) for exemplary genes, such as HTT, SMN2, and MYB, together with a control housekeeping gene, GAPDH or GUSB or PPIA, used for normalization. Briefly, the A673 or K562 cell line was treated with various compounds described herein (e.g., compounds of Formula (I)). After treatment, the levels of the HTT, MYB, or SMN2 mRNA targets were determined from each sample of cell lysate by cDNA synthesis followed by qPCR.

### Materials:

Cells-to-C_{T} 1-step kit: ThermoFisher A25602, Cells-to-C_{T} lysis reagent: ThermoFisher 4391851C, TaqMan™ Fast Virus 1-Step Master Mix: ThermoFisher 4444436
GAPDH: VIC-PL, ThermoFisher 4326317E (Assay: Hs99999905—m1)—used for K562/suspension cell lines
GUSB: VIC-PL, ThermoFisher 4326320E (Assay: Hs99999908—m1)—used for K562/suspension cell lines
PPIA: VIC-PL, ThermoFisher 4326316E (Assay: Hs99999904—m1)—used for A673/adherent cell lines

### Probe/Primer Sequences

Canonical junction (CJ)
HTT Primer 1:
TCCTCCTGAGAAAGAGAAGGAC
HTT Primer 2:
GCCTGGAGATCCAGACTCA
HTT CY5-Probe:
/5Cy5/TGGCAACCCTTGAGGCCCTGTCCT/3IAbRQSp/
MYB Primer 1:
CCTCATTGGTCACAAATTGACTG
MYB Primer 2:
TGGAGAGCTTTCTAAGATTGACC
MYB CY5-Probe:
/5Cy5/AGGAAAATACTGTTTTTAGAACCCCAG/3IAbRQSp/
Alternative junction (AJ)
HTT Primer 1:
TCCTGAGAAAGAGAAGGACATTG
HTT Primer 2:
CTGTGGGCTCCTGTAGAAATC
HTT FAM-Probe:
/56-FAM/TGGCAACCC/ZEN/TTGAGAGGCAAGCCCT/3IABKFQ/
MYB Primer 1:
CAACACCATTICATAGAGACCAGAC
MYB Primer 2:
GTTCTAAAATCATCCCTTGGCTTCTAAT
MYB FAM-Probe:
/56-FAM/AAATACTGT/ZEN/ATAGGACCTCTTCTGACATCC/
3IABKFQ/

### Description:

The A673 cell line was cultured in DMEM with 10% FBS. Cells were diluted with full growth media and plated in a 96-well plate (15,000 cells in 100 ul media per well). The plate was incubated at 37° C. with 5% CO₂ for 24 hours to allow cells to adhere. An 11-point 3-fold serial dilution of the compounds was made in DMSO then diluted in media in an intermediate plate. Compounds were transferred from the intermediate plate to the cell plate with the top dose at a final concentration of 10 uM in the well. Final DMSO concentration was kept at or below 0.25%. The cell plate was returned to the incubator at 37° C. with 5% CO₂ for an additional 24 hours.

The K562 cell line was cultured in IMDM with 10% FBS. For K562, cells were diluted with full growth media and plated in either a 96-well plate (50,000 cells in 50 uL media per well) or a 384-well plate (8,000-40,000 cells in 45 uL media per well). An 11-point 3-fold serial dilution of the compounds were made in DMSO then diluted in media in an intermediate plate. Compound was transferred from the intermediate plate to the cell plate with the top dose at a final concentration of 10 uM in the well. Final DMSO concentration was kept at or below 0.25%. Final volume was 100 uL for 96-well plate and 50 uL for 384-well plate. The cell plate was then placed in an incubator at 37° C. with 5% CO₂ for 24 hours.

The cells were then gently washed with 50 uL-100 uL cold PBS before proceeding to addition of lysis buffer. 30 uL-50 μL of room temperature lysis buffer with DNAse I (and optionally RNAsin) was added to each well. Cells were shaken/mixed thoroughly at room temperature for 5-10 minutes for lysis to take place and then 3 uL-5 μL of room temperature stop solution was added and wells were shaken/mixed again. After 2-5 minutes, the cell lysate plate was transferred to ice for RT-qPCR reaction setup. The lysates could also be frozen at −80° C. for later use.

In some cases, a direct lysis buffer was used. An appropriate volume of 3× lysis buffer (10 mM Tris, 150 mM NaCl, 1.5%-2.5% Igepal and 0.1-1 U/uL RNAsin, pH 7.4) was directly added to either K562 or A673 cells in media and mixed by pipetting 3 times. The plates were then incubated at room temperature with shaking/rocking for 20-50 minutes to allow for lysis to take place. After this time, the cell lysate plate was transferred to ice to set up for the RT-qPCR reactions. The lysates could also be frozen at −80° C. for later use.

To set up 10 uL RT-qPCR reactions, cell lysates were transferred to 384-well qPCR plates containing the master mix according to the table below. The plates were sealed, gently vortexed, and spun down before the run. The volumes were adjusted accordingly in some instances where the reaction was carried in 20 μL. The table below summarizes the components of the RT-qPCR reactions:

| Component | 1X |
|---|---|
| Taqman 1-step RT-qPCR mix (4X) | 2.5 |
| 20X AJ Primers + Probe (FAM) | 0.5 |
| 20X CJ Primers + Probe (CY5) | 0.5 |
| 20X PPIA Control (VIC) | 0.5 |
| Cell lysate (1X) | 1-2 |
| H₂O | 4-5 |
| Total volume | 10 |

The RT-qPCR reaction was performed using a QuantStudio (ThermoFisher) under the following fast cycling conditions. All samples and standards were analyzed at least in duplicate. In some instances, bulk room temperature (RT) step of 5-10 minutes was completed for all plates before proceeding with qPCR. The table below summarizes the PCR cycle:

| Step | # cycles | Temp. | Time |
|---|---|---|---|
| RT step | 1 | 50° C. | 5 min |
| RT inactivation/initial | 1 | 95° C. | 20 sec |
| denaturation | | | |
| Amplification | 40 | 95° C. | 3 sec |
| | | 60° C. | 30 sec |

The data analysis was performed by first determining the ΔCt vs the housekeeper gene. This ΔCt was then normalized against the DMSO control (ΔΔCt) and converted to RQ (relative quantification) using the 2\hat{ }(−ΔΔCt) equation. The RQ were then converted to a percentage response by arbitrarily setting an assay window of 3.5 and 4.0 ΔCt for HTT-CJ and MYB-CJ respectively and an assay window of 9 and 3 ΔCt for HTT-AJ and MYB-AJ in 96 well format (50,000 K562 cells/well and 15,000 A673 cells per well) and an assay window of 3 and 4 ΔCt for HTT-CJ and MYB-CJ respectively and an assay window of 5 and 3 ΔCt for HTT-AJ and MYB-AJ respectively in 384 well format (8,000 K562 cells/well example). These assay windows correspond to the maximal modulation observed at high concentration of the most active compounds. The percentage response was then fitted to the 4 parametric logistic equation to evaluate the concentration dependence of compound treatment. The increase in AJ mRNA is reported as AC₅₀ (compound concentration having 50% response in AJ increase) while the decrease in CJ mRNA levels is reported as IC₅₀ (compound concentration having 500 response in CJ decrease).

A summary of these results is illustrated in Table 3, wherein “A” represents an AC₅₀/IC₅₀ of less than 100 nM; “B” represents an AC₅₀/IC₅₀ of between 100 nM and 1 μM; and “C” represents an AC₅₀/IC₅₀ of between 1 μM and 10 μM; and “D” represents an AC₅₀/IC₅₀ of greater than 10 μM.

TABLE 3
Modulation of RNA Splicing by Exemplary Compounds

| Cmpnd | HTT | HTT | MYB | MYB |
|---|---|---|---|---|
| No. | CJ | AJ | CJ | AJ |
| 100 | A | B | A | C |
| 101 | A | A | A | A |
| 102 | A | A | A | A |
| 104 | C | C | C | C |
| 106 | C | C | C | B |
| 107 | B | B | B | B |
| 108 | C | C | C | C |
| 109 | A | B | A | B |
| 115 | C | C | B | B |
| 116 | C | C | B | C |
| 117 | C | C | B | B |
| 118 | C | C | B | C |
| 119 | B | C | B | B |
| 120 | C | C | B | B |
| 121 | C | C | B | B |
| 124 | A | B | B | B |
| 125 | B | B | B | A |
| 126 | B | B | B | B |
| 127 | A | A | A | A |
| 129 | B | B | B | B |
| 130 | D | D | D | D |
| 131 | C | D | C | C |
| 132 | D | D | D | D |
| 133 | C | C | C | B |
| 134 | C | C | B | B |
| 135 | C | C | B | B |
| 136 | C | C | B | B |
| 137 | B | C | B | B |
| 138 | B | B | B | B |
| 139 | A | A | A | A |
| 140 | A | B | A | B |
| 141 | C | C | B | C |
| 142 | B | B | B | B |
| 143 | D | D | D | D |
| 144 | D | D | D | D |
| 145 | D | D | D | D |
| 146 | C | C | C | C |
| 148 | C | C | B | B |
| 149 | D | D | C | D |
| 150 | A | A | A | A |
| 151 | B | B | B | B |
| 152 | D | D | D | D |
| 153 | A | A | A | A |
| 154 | A | A | B | A |
| 155 | A | A | A | A |
| 156 | A | A | A | A |
| 157 | A | A | A | A |
| 158 | A | A | A | A |
| 159 | C | C | C | C |
| 160 | B | B | B | B |
| 161 | D | D | D | D |
| 162 | D | D | D | D |
| 163 | D | D | C | C |
| 164 | B | B | B | B |
| 165 | C | C | C | C |
| 166 | D | D | D | D |
| 167 | D | D | D | D |
| 168 | D | D | D | D |
| 169 | D | D | D | D |
| 170 | A | B | B | B |
| 171 | A | A | A | A |
| 172 | A | B | A | A |
| 173 | A | A | A | A |
| 174 | D | D | D | D |
| 175 | A | A | A | A |
| 176 | C | C | C | C |
| 177 | C | C | C | C |
| 178 | B | B | B | B |
| 179 | A | A | B | A |
| 180 | A | B | A | A |
| 182 | C | D | C | D |
| 184 | B | B | B | B |
| 185 | C | C | C | C |
| 186 | A | A | B | A |
| 187 | A | A | B | A |
| 188 | B | B | B | B |
| 189 | C | B | B | B |
| 190 | D | D | D | D |
| 191 | D | D | D | D |
| 193 | C | C | C | C |
| 194 | A | A | A | A |
| 195 | B | B | B | A |
| 196 | D | D | D | D |
| 197 | A | A | B | B |
| 198 | A | A | A | A |
| 199 | B | B | B | B |
| 200 | C | C | C | C |
| 201 | B | B | B | B |
| 202 | A | A | A | A |
| 203 | A | A | B | B |
| 204 | C | C | C | C |
| 205 | C | C | C | C |
| 206 | C | C | C | C |
| 207 | C | C | C | C |
| 208 | D | D | D | C |
| 209 | A | A | A | A |
| 210 | B | C | C | C |
| 211 | A | A | A | A |
| 212 | A | A | B | B |
| 214 | A | A | B | B |
| 215 | A | A | A | A |
| 216 | A | A | A | A |
| 217 | A | A | A | A |
| 218 | D | D | D | C |
| 219 | A | A | A | A |
| 220 | A | A | A | A |
| 221 | A | A | A | A |
| 222 | A | A | A | A |
| 223 | A | A | A | A |
| 224 | A | A | A | A |
| 225 | A | A | A | A |
| 226 | A | A | A | B |
| 228 | A | A | B | A |
| 229 | A | A | A | A |
| 230 | A | A | B | B |
| 231 | A | A | A | A |
| 232 | A | A | A | A |
| 233 | A | A | A | A |
| 234 | B | A | B | A |
| 235 | A | A | A | A |
| 236 | A | A | A | A |
| 237 | A | A | A | A |
| 238 | A | A | A | A |
| 241 | A | A | A | A |
| 245 | D | D | D | D |
| 246 | A | A | A | A |
| 247 | A | B | A | A |
| 248 | C | B | C | C |
| 249 | A | A | A | A |
| 250 | A | A | A | A |
| 251 | D | D | D | D |
| 252 | A | B | B | B |
| 253 | A | A | A | A |
| 254 | A | A | A | A |
| 255 | A | A | A | A |
| 256 | A | A | A | A |
| 257 | A | A | A | A |
| 258 | A | A | B | B |
| 259 | A | A | A | A |
| 260 | A | A | A | A |
| 275 | A | A | A | A |
| 284 | A | A | A | B |
| 285 | A | A | A | A |
| 286 | A | A | A | A |
| 287 | A | A | A | A |
| 288 | A | A | A | A |
| 289 | A | A | A | A |
| 290 | A | A | A | A |
| 291 | B | B | B | B |
| 292 | B | B | A | B |
| 293 | A | A | A | A |
| 294 | A | A | A | A |
| 295 | A | A | A | A |
| 296 | A | A | B | A |
| 297 | C | C | C | B |
| 298 | A | A | A | A |
| 299 | A | B | A | A |
| 300 | A | A | A | A |
| 302 | B | B | B | B |
| 303 | A | A | A | A |
| 304 | A | A | A | A |
| 305 | A | A | A | A |
| 306 | A | A | A | A |
| 307 | A | A | A | A |
| 308 | A | A | A | A |
| 309 | A | A | A | A |
| 310 | A | A | A | A |
| 311 | A | A | B | B |
| 312 | A | A | A | A |
| 313 | A | A | A | A |
| 314 | A | A | A | A |
| 315 | A | A | A | A |
| 316 | A | A | A | A |
| 317 | A | A | A | A |
| 318 | A | A | A | A |
| 319 | A | A | A | A |
| 320 | A | A | A | A |
| 321 | A | A | A | A |
| 322 | A | A | A | A |
| 323 | A | A | A | A |
| 324 | A | A | A | A |
| 325 | A | A | A | A |
| 326 | A | A | A | A |
| 327 | A | A | A | A |
| 328 | A | A | A | A |
| 329 | A | A | A | A |
| 330 | A | A | A | A |
| 331 | B | B | A | B |
| 332 | B | B | B | B |
| 333 | B | B | B | B |
| 334 | B | B | B | B |
| 335 | C | C | C | C |
| 336 | A | B | B | B |
| 337 | B | B | A | A |
| 338 | B | C | A | B |
| 339 | B | B | B | B |
| 340 | B | B | B | B |
| 341 | B | B | B | B |
| 342 | B | B | B | B |
| 343 | C | C | C | C |
| 344 | C | C | C | C |
| 346 | C | C | C | C |
| 347 | A | A | A | A |
| 348 | B | B | A | A |
| 349 | A | A | A | A |
| 350 | A | A | B | B |
| 351 | A | A | A | A |
| 352 | A | A | A | A |
| 353 | A | A | A | A |
| 354 | A | A | A | A |
| 355 | A | A | A | A |
| 356 | A | A | A | A |
| 357 | A | A | A | A |
| 358 | A | A | A | A |
| 360 | A | A | A | A |
| 362 | A | A | A | A |
| 363 | A | A | A | A |
| 364 | D | D | C | C |
| 368 | B | B | A | A |
| 369 | B | B | B | B |
| 370 | B | C | B | B |
| 372 | A | A | A | A |
| 373 | A | A | A | A |
| 374 | A | A | A | A |
| 375 | A | A | A | A |
| 376 | A | B | A | A |
| 377 | A | A | A | A |
| 378 | A | A | A | A |
| 379 | A | B | B | B |
| 380 | A | B | A | A |
| 382 | B | B | A | A |
| 383 | A | A | A | A |
| 384 | C | C | B | B |
| 385 | A | B | B | B |
| 386 | D | D | B | C |
| 387 | A | A | A | A |
| 388 | A | A | A | A |
| 389 | A | B | A | A |
| 390 | A | A | A | A |
| 391 | A | A | A | A |
| 392 | A | A | A | A |
| 392 | A | A | A | A |
| 393 | B | B | A | A |
| 395 | B | B | B | B |
| 396 | A | A | B | B |
| 397 | C | C | B | B |
| 398 | C | C | C | C |
| 399 | D | D | C | C |
| 403 | B | B | B | A |
| 404 | A | A | B | B |
| 405 | C | C | C | C |
| 406 | B | C | C | B |
| 407 | B | C | B | B |
| 408 | B | C | B | B |
| 409 | B | B | B | B |
| 410 | B | B | B | B |
| 411 | C | D | B | B |
| 412 | B | B | B | B |
| 413 | A | A | A | A |
| 414 | A | B | C | C |
| 415 | A | A | A | A |
| 416 | A | A | A | A |
| 417 | B | C | B | B |
| 418 | B | B | B | B |
| 419 | B | B | B | B |
| 420 | B | C | B | B |
| 421 | B | B | B | B |
| 422 | B | B | B | B |
| 424 | C | C | C | C |
| 425 | C | C | C | C |
| 432 | A | A | A | B |
| 433 | A | A | B | B |
| 434 | A | A | B | B |
| 435 | B | B | B | B |
| 436 | A | A | A | A |
| 437 | A | A | B | B |
| 438 | A | A | B | A |
| 439 | A | A | B | B |
| 440 | A | B | B | B |
| 441 | A | B | B | B |
| 444 | A | B | B | B |
| 445 | C | C | C | C |
| 448 | B | C | B | C |
| 449 | C | C | B | B |
| 450 | C | C | C | C |
| 456 | A | A | A | A |
| 458 | C | C | B | B |
| 482 | A | B | A | A |
| 484 | D | D | B | B |
| 486 | C | C | B | B |
| 488 | A | A | A | A |
| 489 | A | A | A | A |
| 491 | A | A | A | A |
| 492 | B | B | C | B |
| 493 | A | A | A | A |
| 495 | A | A | A | A |
| 496 | B | A | B | A |
| 497 | A | A | A | A |
| 498 | A | A | B | A |
| 502 | C | C | C | C |
| 503 | A | A | A | A |
| 504 | B | B | B | B |
| 505 | B | B | A | A |
| 506 | A | B | A | A |
| 507 | B | C | A | A |
| 508 | D | D | C | C |
| 509 | D | D | D | D |
| 510 | A | B | A | A |
| 511 | B | B | A | A |
| 512 | B | B | A | A |
| 513 | C | C | A | A |
| 514 | B | C | A | A |
| 515 | A | A | A | A |
| 516 | B | B | A | B |
| 517 | B | B | A | A |
| 518 | C | C | A | B |
| 519 | D | D | B | B |
| 520 | B | B | B | A |
| 521 | A | B | A | A |
| 522 | C | D | C | C |
| 523 | B | B | A | A |
| 524 | D | D | D | D |
| 525 | B | B | A | A |
| 526 | B | C | A | B |
| 527 | A | B | A | A |
| 528 | B | C | A | A |
| 529 | B | B | A | A |
| 530 | C | C | A | A |
| 531 | C | D | A | A |
| 532 | B | B | A | A |
| 533 | C | C | B | B |
| 534 | D | D | B | B |
| 535 | A | B | A | A |
| 536 | A | A | A | A |
| 537 | A | B | A | A |
| 538 | C | C | C | C |
| 539 | A | A | A | A |
| 540 | C | B | A | A |
| 541 | C | C | C | C |
| 542 | C | C | A | A |
| 543 | A | A | A | A |
| 544 | B | B | B | B |
| 652 | B | B | A | A |
| 653 | A | B | A | A |
| 660 | B | B | A | A |
| 661 | B | C | B | B |
| 668 | B | C | A | A |
| 669 | B | B | A | A |
| 676 | B | B | A | A |
| 677 | B | C | A | A |
| 705 | B | B | A | B |
| 706 | B | C | A | A |
| 707 | B | B | A | A |
| 712 | C | C | A | A |
| 717 | B | B | A | A |
| 722 | A | B | A | A |
| 723 | A | B | A | A |
| 728 | B | B | A | A |
| 729 | A | A | A | A |
| 732 | D | D | C | D |
| 737 | C | C | B | B |
| 742 | B | D | B | B |
| 743 | D | C | C | D |
| 744 | A | A | A | A |
| 745 | B | B | A | A |
| 748 | D | D | B | C |
| 749 | D | D | B | B |
| 750 | B | B | A | B |
| 751 | D | D | B | B |
| 752 | C | C | A | A |
| 753 | C | C | B | B |
| 756 | B | B | B | B |
| 757 | C | C | A | B |
| 758 | B | C | B | B |
| 760 | A | B | A | B |
| 763 | A | B | A | A |
| 768 | C | C | B | B |
| 770 | B | B | A | A |
| 773 | C | C | A | A |
| 779 | C | C | A | A |
| 780 | C | C | B | B |
| 781 | D | D | C | C |
| 782 | C | D | B | B |
| 783 | D | D | B | B |
| 784 | C | D | A | A |
| 785 | C | D | A | B |
| 786 | B | B | B | B |
| 787 | C | C | B | B |
| 788 | C | C | C | B |
| 789 | B | B | A | A |
| 790 | C | D | C | C |
| 791 | C | D | C | C |
| 792 | B | C | B | B |
| 793 | C | C | B | B |
| 794 | D | C | B | B |
| 795 | D | D | C | C |
| 796 | B | B | A | A |
| 797 | C | B | A | A |
| 798 | D | D | C | B |
| 799 | B | B | A | B |
| 800 | A | A | A | A |
| 801 | C | C | B | C |
| 802 | B | B | B | B |
| 803 | B | B | A | A |
| 804 | B | B | B | B |
| 805 | A | B | A | A |
| 806 | D | C | A | B |
| 807 | D | C | A | A |
| 808 | C | C | C | C |
| 809 | C | C | C | C |
| 810 | C | C | B | B |
| 811 | C | C | A | A |
| 812 | C | C | B | B |
| 813 | D | D | B | B |
| 814 | B | C | B | B |
| 815 | C | C | B | B |
| 816 | C | C | B | A |
| 817 | B | C | A | A |
| 818 | B | C | A | A |
| 819 | C | C | B | B |
| 820 | D | D | C | C |
| 821 | D | D | C | D |
| 822 | C | D | B | C |
| 823 | C | C | B | B |
| 824 | B | C | B | B |
| 825 | C | C | B | B |
| 826 | C | C | B | A |
| 827 | C | C | B | B |
| 828 | B | C | A | A |
| 829 | B | B | A | B |
| 830 | C | D | B | B |
| 831 | B | C | A | A |
| 832 | D | D | B | C |
| 833 | C | C | C | C |
| 834 | C | C | B | B |
| 835 | C | C | B | B |
| 836 | C | D | D | C |
| 837 | B | C | C | C |
| 838 | D | D | B | B |
| 924 | D | C | C | C |
| 925 | C | B | C | C |
| 929 | C | B | A | A |
| 930 | C | D | B | B |

Additional studies were carried out for a larger panel of genes using the protocol provided above. The junction between flanking upstream and downstream exons was used to design canonical junction Qpcr assays. At least one of the forward primer, reverse primer or the CY5-labeled 5′ nuclease probe (with 3′ quencher such as ZEN/Iowa Black FQ) was designed to overlap with the exon junction to capture the CJ Mrna transcript. BLAST was used to confirm the specificity of the probeset and parameters suCH As melting temperature, GC content, amplicon size, and primer dimer formation are considered during their design. Data for the decrease in CJ Mrna levels for four exemplary genes (HTT, SMN2, MYB, and Target C) analyzed in this panel are reported as IC₅₀ (compound concentration having 5000 response in CJ decrease).

A summary of the results from the panel is illustrated in Table 4, wherein “A” represents an IC₅₀ of less than 100 Nm; “B” represents an IC₅₀ of between 100 Nm and 1 μM; and “C” represents an IC₅₀ of between 1 μM and 10 μM; and “D” represents an IC₅₀ of greater than 10 μM.

TABLE 4
Modulation of RNA Splicing by Exemplary Compounds

| Cmpnd | | | Target | |
|---|---|---|---|---|
| No | HTT | MYB | C | SMN2 |
| 100 | A | A | A | A |
| 101 | A | A | A | A |
| 102 | A | A | A | A |
| 104 | C | C | C | B |
| 106 | C | C | C | B |
| 107 | B | B | C | A |
| 108 | C | C | C | A |
| 109 | A | A | B | A |
| 115 | C | B | C | A |
| 116 | C | B | C | A |
| 117 | C | B | C | A |
| 118 | C | B | C | A |
| 119 | B | B | C | A |
| 120 | C | B | C | A |
| 121 | C | B | C | C |
| 124 | A | B | B | A |
| 125 | B | B | B | A |
| 126 | B | B | A | A |
| 127 | A | A | A | A |
| 129 | B | B | B | A |
| 130 | D | D | D | B |
| 131 | C | C | D | A |
| 132 | D | D | D | D |
| 133 | C | C | C | A |
| 134 | C | B | C | A |
| 135 | C | B | C | A |
| 136 | C | B | C | A |
| 137 | B | B | C | A |
| 138 | B | B | C | A |
| 139 | A | A | B | A |
| 140 | A | A | B | A |
| 141 | C | B | D | B |
| 142 | B | B | B | A |
| 143 | D | D | D | D |
| 144 | D | D | D | D |
| 145 | D | D | D | D |
| 146 | C | C | C | B |
| 148 | C | B | C | A |
| 149 | D | C | D | B |
| 150 | A | A | A | A |
| 151 | B | B | B | B |
| 152 | D | D | D | C |
| 153 | A | A | A | A |
| 154 | A | B | A | A |
| 155 | A | A | B | A |
| 156 | A | A | B | A |
| 157 | A | A | B | A |
| 158 | A | A | A | A |
| 159 | C | C | C | B |
| 160 | B | B | C | A |
| 161 | D | D | D | C |
| 162 | D | D | D | C |
| 163 | D | C | D | B |
| 164 | B | B | B | B |
| 165 | C | C | C | C |
| 166 | D | D | D | D |
| 167 | D | D | D | D |
| 168 | D | D | D | D |
| 169 | D | D | D | C |
| 170 | A | B | B | A |
| 171 | A | A | B | A |
| 172 | A | A | B | A |
| 173 | A | A | A | A |
| 174 | D | D | D | D |
| 175 | A | A | A | A |
| 176 | C | C | C | C |
| 177 | C | C | C | C |
| 178 | B | B | C | A |
| 179 | A | B | B | A |
| 180 | A | A | B | A |
| 182 | C | C | D | C |
| 184 | B | B | B | A |
| 185 | C | C | C | B |
| 186 | A | B | A | A |
| 187 | A | B | A | A |
| 188 | B | B | C | A |
| 189 | C | B | C | A |
| 190 | D | D | D | A |
| 191 | D | D | D | D |
| 193 | C | C | C | C |
| 194 | A | A | B | A |
| 195 | B | B | B | A |
| 196 | D | D | D | C |
| 197 | A | B | B | A |
| 198 | A | A | B | A |
| 199 | B | B | B | B |
| 200 | C | C | C | C |
| 201 | B | B | B | A |
| 202 | A | A | A | A |
| 203 | A | B | A | A |
| 204 | C | C | D | B |
| 205 | C | C | C | C |
| 206 | C | C | C | C |
| 207 | C | C | C | C |
| 208 | D | D | D | C |
| 209 | A | A | A | A |
| 210 | B | C | C | A |
| 211 | A | A | B | A |
| 212 | A | B | B | A |
| 214 | A | B | A | A |
| 215 | A | A | A | A |
| 216 | A | A | A | A |
| 217 | A | A | A | A |
| 218 | D | D | D | B |
| 219 | A | A | A | A |
| 220 | A | A | A | A |
| 221 | A | A | A | A |
| 222 | A | A | A | A |
| 223 | A | A | A | A |
| 224 | A | A | A | A |
| 225 | A | A | A | A |
| 226 | A | A | A | A |
| 228 | A | B | B | A |
| 229 | A | A | B | A |
| 230 | A | B | B | A |
| 231 | A | A | A | A |
| 232 | A | A | A | A |
| 233 | A | A | A | A |
| 234 | B | B | B | A |
| 235 | A | A | B | A |
| 236 | A | A | B | A |
| 237 | A | A | A | A |
| 238 | A | A | A | A |
| 24 | A | A | A | A |
| 245 | D | D | D | C |
| 246 | A | A | A | A |
| 247 | A | A | B | A |
| 248 | C | C | C | B |
| 249 | A | A | A | A |
| 250 | A | A | A | A |
| 251 | D | D | D | D |
| 252 | A | B | B | A |
| 253 | A | A | A | A |
| 254 | A | A | A | A |
| 255 | A | A | A | A |
| 256 | A | A | A | A |
| 257 | A | A | A | A |
| 258 | A | B | B | A |
| 259 | A | A | A | A |
| 260 | A | A | A | A |
| 275 | A | A | A | A |
| 284 | A | A | B | A |
| 285 | A | A | A | A |
| 286 | A | A | A | A |
| 287 | A | A | A | A |
| 288 | A | A | B | A |
| 289 | A | A | A | A |
| 290 | A | A | A | A |
| 291 | B | B | B | A |
| 292 | B | A | B | A |
| 293 | A | A | A | A |
| 294 | A | A | A | A |
| 295 | A | A | B | A |
| 296 | A | B | A | A |
| 297 | C | C | D | B |
| 298 | A | A | B | A |
| 299 | A | A | C | A |
| 300 | A | A | A | A |
| 302 | B | B | C | A |
| 303 | A | A | A | A |
| 304 | A | A | A | A |
| 305 | A | A | A | A |
| 306 | A | A | A | A |
| 307 | A | A | A | A |
| 308 | A | A | A | A |
| 309 | A | A | A | A |
| 310 | A | A | A | A |
| 311 | A | B | B | A |
| 312 | A | A | A | A |
| 313 | A | A | A | A |
| 314 | A | A | A | A |
| 315 | A | A | A | A |
| 316 | A | A | A | A |
| 317 | A | A | A | A |
| 318 | A | A | A | A |
| 319 | A | A | A | A |
| 320 | A | A | A | A |
| 321 | A | A | A | A |
| 322 | A | A | A | A |
| 323 | A | A | A | A |
| 324 | A | A | A | A |
| 325 | A | A | A | A |
| 326 | A | A | A | A |
| 327 | A | A | A | A |
| 328 | A | A | A | A |
| 329 | A | A | A | A |
| 330 | A | A | A | A |
| 331 | B | A | B | A |
| 332 | B | B | B | A |
| 333 | B | B | B | B |
| 334 | B | B | B | B |
| 335 | C | C | C | C |
| 336 | A | B | B | A |
| 337 | B | A | D | A |
| 338 | B | A | C | A |
| 339 | B | B | B | B |
| 340 | B | B | B | B |
| 341 | B | B | B | A |
| 342 | B | B | B | A |
| 343 | C | C | D | A |
| 344 | C | C | D | A |
| 346 | C | C | C | B |
| 347 | A | A | A | A |
| 348 | B | A | B | A |
| 349 | A | A | A | A |
| 350 | A | B | B | A |
| 351 | A | A | A | A |
| 352 | A | A | A | A |
| 353 | A | A | A | A |
| 354 | A | A | A | A |
| 355 | A | A | B | A |
| 356 | A | A | B | A |
| 357 | A | A | A | A |
| 358 | A | A | B | A |
| 360 | A | A | A | A |
| 362 | A | A | B | A |
| 363 | A | A | A | A |
| 364 | D | C | D | B |
| 368 | B | A | C | A |
| 369 | B | B | C | A |
| 370 | B | B | C | A |
| 372 | A | A | A | A |
| 373 | A | A | A | A |
| 374 | A | A | A | A |
| 375 | A | A | A | A |
| 376 | A | A | B | A |
| 377 | A | A | A | A |
| 378 | A | A | A | A |
| 379 | A | B | B | A |
| 380 | A | A | C | A |
| 382 | B | A | C | A |
| 383 | A | A | B | A |
| 384 | C | B | D | A |
| 385 | A | B | B | A |
| 386 | D | B | D | A |
| 387 | A | A | A | A |
| 388 | A | A | A | A |
| 389 | A | A | B | A |
| 390 | A | A | A | A |
| 391 | A | A | A | A |
| 392 | A | A | A | A |
| 392 | A | A | A | A |
| 393 | B | A | B | A |
| 395 | B | B | C | A |
| 396 | A | B | B | A |
| 397 | C | B | D | A |
| 398 | C | C | D | A |
| 399 | D | C | D | B |
| 403 | B | B | C | A |
| 404 | A | B | A | A |
| 405 | C | C | C | C |
| 406 | B | C | C | B |
| 407 | B | B | C | A |
| 408 | B | B | C | A |
| 409 | B | B | C | A |
| 410 | B | B | B | A |
| 411 | C | B | C | A |
| 412 | B | B | B | B |
| 413 | A | A | A | A |
| 414 | A | C | C | B |
| 415 | A | A | A | A |
| 416 | A | A | A | A |
| 417 | B | B | C | A |
| 418 | B | B | B | B |
| 419 | B | B | B | A |
| 420 | B | B | C | A |
| 421 | B | B | B | B |
| 422 | B | B | C | A |
| 424 | C | C | C | A |
| 425 | C | C | D | B |
| 432 | A | A | A | A |
| 433 | A | B | B | A |
| 434 | A | B | B | A |
| 435 | B | B | B | A |
| 436 | A | A | A | A |
| 437 | A | B | B | B |
| 438 | A | B | B | A |
| 439 | A | B | B | A |
| 440 | A | B | B | A |
| 441 | A | B | B | A |
| 444 | A | B | B | A |
| 445 | C | C | C | B |
| 448 | B | B | C | A |
| 449 | C | B | C | A |
| 450 | C | C | C | C |
| 456 | A | A | B | A |
| 458 | C | B | D | A |
| 482 | A | A | C | A |
| 484 | D | B | C | A |
| 486 | C | B | C | A |
| 488 | A | A | A | A |
| 489 | A | A | B | A |
| 491 | A | A | A | A |
| 492 | B | C | C | A |
| 493 | A | A | A | A |
| 495 | A | A | B | A |
| 496 | B | B | B | A |
| 497 | A | A | A | A |
| 498 | A | B | B | A |
| 502 | C | C | C | B |
| 503 | A | A | B | A |
| 504 | B | B | B | A |
| 505 | B | A | B | A |
| 506 | A | A | B | A |
| 507 | B | A | C | A |
| 508 | D | C | D | B |
| 509 | D | D | D | B |
| 510 | A | A | B | A |
| 511 | B | A | C | A |
| 512 | B | A | C | A |
| 513 | C | A | C | A |
| 514 | B | A | C | A |
| 515 | A | A | B | A |
| 516 | B | A | B | A |
| 517 | B | A | B | A |
| 518 | C | A | D | A |
| 519 | D | B | D | A |
| 520 | B | B | C | A |
| 521 | A | A | B | A |
| 522 | C | C | D | A |
| 523 | B | A | C | A |
| 524 | D | D | D | C |
| 525 | B | A | C | A |
| 526 | B | A | C | A |
| 527 | A | A | B | A |
| 528 | B | A | C | A |
| 529 | B | A | C | A |
| 530 | C | A | D | A |
| 531 | C | A | D | A |
| 532 | B | A | D | A |
| 533 | C | B | D | A |
| 534 | D | B | D | A |
| 535 | A | A | C | A |
| 536 | A | A | A | A |
| 537 | A | A | B | A |
| 538 | C | C | C | B |
| 539 | A | A | B | A |
| 540 | C | A | D | A |
| 541 | C | C | C | C |
| 542 | C | A | D | A |
| 543 | A | A | B | A |
| 544 | B | B | C | A |
| 652 | B | A | D | A |
| 653 | A | A | B | A |
| 660 | B | A | C | A |
| 661 | B | B | B | A |
| 668 | B | A | C | A |
| 669 | B | A | C | A |
| 676 | B | A | D | A |
| 677 | B | A | C | A |
| 705 | B | A | B | |
| 706 | B | A | D | A |
| 707 | B | A | B | A |
| 712 | C | A | D | A |
| 717 | B | A | B | A |
| 722 | A | A | B | A |
| 723 | A | A | B | A |
| 728 | B | A | C | A |
| 729 | A | A | A | A |
| 732 | D | C | D | B |
| 737 | C | B | C | A |
| 742 | B | B | D | A |
| 743 | D | C | C | C |
| 744 | A | A | C | A |
| 745 | B | A | C | A |
| 748 | D | B | D | B |
| 749 | D | B | C | A |
| 750 | B | A | B | A |
| 751 | D | B | D | A |
| 752 | C | A | D | A |
| 753 | C | B | D | A |
| 756 | B | B | B | A |
| 757 | C | A | C | A |
| 758 | B | B | C | A |
| 760 | A | A | B | |
| 763 | A | A | C | A |
| 768 | C | B | C | A |
| 770 | B | A | B | |
| 773 | C | A | C | A |
| 779 | C | A | C | A |
| 780 | C | B | C | A |
| 781 | D | C | D | B |
| 782 | C | B | D | A |
| 783 | D | B | D | A |
| 784 | C | A | D | A |
| 785 | C | A | C | A |
| 786 | B | B | B | A |
| 787 | C | B | C | A |
| 788 | C | C | C | A |
| 789 | B | A | B | A |
| 790 | C | C | D | |
| 791 | C | C | D | |
| 792 | B | B | C | |
| 793 | C | B | D | |
| 794 | D | B | D | |
| 795 | D | C | D | |
| 796 | B | A | D | |
| 797 | C | A | D | |
| 798 | D | C | D | |
| 799 | B | A | C | |
| 800 | A | A | B | |
| 801 | C | B | C | |
| 802 | B | B | B | |
| 803 | B | A | B | A |
| 804 | B | B | B | |
| 805 | A | A | B | |
| 806 | D | A | D | |
| 807 | D | A | D | |
| 808 | C | C | C | B |
| 809 | C | C | C | C |
| 810 | C | B | C | A |
| 811 | C | A | D | A |
| 812 | C | B | C | A |
| 813 | D | B | D | A |
| 814 | B | B | D | A |
| 815 | C | B | D | A |
| 816 | C | B | D | A |
| 817 | B | A | C | A |
| 818 | B | A | C | A |
| 819 | C | B | C | A |
| 820 | D | C | D | B |
| 821 | D | C | D | B |
| 822 | C | B | C | B |
| 823 | C | B | D | A |
| 824 | B | B | C | A |
| 825 | C | B | C | A |
| 826 | C | B | C | A |
| 827 | C | B | | A |
| 828 | B | A | | A |
| 829 | B | A | | A |
| 830 | C | B | | A |
| 831 | B | A | | A |
| 832 | D | B | | A |
| 833 | C | C | | B |
| 834 | C | B | | A |
| 835 | C | B | | A |
| 836 | C | D | | B |
| 837 | B | C | | B |
| 838 | D | B | | A |
| 924 | D | C | | B |
| 925 | C | C | | B |
| 929 | C | A | | A |
| 930 | C | B | | A |

### Example 335: Investigating Effect of Exemplary Compounds on Cell Viability

Compounds described herein were screened for toxicity in K562 (human chronic myelogenous leukemia) and SH-SY5Y (human neuroblastoma) cells using a Cell Titer Glo 2.0 assay.

### Materials:

### Promega CellTiter-Glo® 2.0 Cell Viability Assay (cat #G9241)

Corning 384-well TC-treated microplates (cat #3570)

### Description:

Cells were plated at 500 cells/well (K562 cells) in 45 μL of IMDM supplemented with 10% FBS in a 384-well opaque plate. Wells containing only medium were used as a blank control. Test compounds (e.g., compounds of Formula (I) or (II)) were first serially diluted in DMSO then diluted 1:100 with EIDM+10% FBS. The final concentration of DMSO was 0.1% in each well. The cells were incubated for 72 hours at 37° C. and 5% CO₂ before assaying with Cell Titer Glo 2.0 reagent.

A summary of the results for viability is illustrated in Table 4, wherein A represents <100 nM; B represents 100-1000 nM; C represents 1000-9999 nM; and D represents greater than 10 μM in K562 cells.

TABLE 5
Effect of Exemplary Compounds on Cell Viability

| Cmpd | |
|---|---|
| No. | K562 |
| 100 | B |
| 101 | A |
| 102 | A |
| 104 | C |
| 106 | B |
| 107 | B |
| 108 | B |
| 109 | B |
| 110 | |
| 111 | |
| 112 | |
| 115 | B |
| 116 | C |
| 117 | B |
| 118 | C |
| 119 | B |
| 120 | B |
| 121 | C |
| 123 | |
| 124 | B |
| 125 | B |
| 126 | B |
| 127 | B |
| 129 | B |
| 130 | B |
| 131 | B |
| 132 | C |
| 133 | B |
| 134 | C |
| 135 | B |
| 136 | B |
| 137 | B |
| 138 | B |
| 139 | B |
| 140 | B |
| 141 | C |
| 142 | B |
| 143 | B |
| 144 | D |
| 145 | D |
| 146 | C |
| 147 | |
| 148 | B |
| 149 | C |
| 150 | A |
| 151 | |
| 152 | D |
| 153 | B |
| 154 | A |
| 155 | A |
| 156 | B |
| 157 | B |
| 158 | B |
| 159 | C |
| 160 | B |
| 161 | C |
| 162 | C |
| 163 | C |
| 164 | B |
| 165 | B |
| 166 | B |
| 167 | B |
| 168 | D |
| 169 | B |
| 170 | B |
| 171 | B |
| 172 | B |
| 173 | A |
| 174 | A |
| 175 | B |
| 176 | C |
| 177 | C |
| 178 | B |
| 179 | B |
| 180 | A |
| 181 | |
| 182 | D |
| 183 | |
| 184 | B |
| 185 | C |
| 186 | B |
| 187 | B |
| 188 | C |
| 189 | D |
| 190 | C |
| 191 | A |
| 192 | |
| 193 | C |
| 194 | B |
| 195 | B |
| 196 | D |
| 197 | B |
| 198 | B |
| 199 | B |
| 200 | C |
| 201 | B |
| 202 | A |
| 203 | B |
| 204 | D |
| 205 | C |
| 206 | C |
| 207 | C |
| 208 | D |
| 209 | C |
| 210 | A |
| 211 | B |
| 212 | B |
| 214 | A |
| 215 | A |
| 216 | B |
| 217 | B |
| 218 | C |
| 219 | A |
| 220 | A |
| 221 | B |
| 222 | B |
| 223 | A |
| 224 | B |
| 225 | B |
| 226 | B |
| 228 | B |
| 229 | B |
| 230 | B |
| 231 | A |
| 232 | A |
| 233 | B |
| 234 | A |
| 235 | B |
| 236 | B |
| 237 | B |
| 238 | A |
| 241 | A |
| 245 | D |
| 246 | B |
| 247 | B |
| 248 | C |
| 249 | A |
| 250 | B |
| 251 | B |
| 252 | |
| 253 | A |
| 254 | A |
| 255 | A |
| 256 | A |
| 257 | B |
| 258 | C |
| 259 | B |
| 260 | B |
| 283 | |
| 284 | B |
| 285 | A |
| 286 | A |
| 287 | A |
| 288 | B |
| 289 | A |
| 290 | A |
| 291 | B |
| 292 | B |
| 293 | A |
| 294 | A |
| 295 | B |
| 296 | B |
| 297 | D |
| 298 | B |
| 299 | B |
| 300 | B |
| 301 | |
| 302 | C |
| 303 | A |
| 304 | A |
| 305 | A |
| 306 | A |
| 307 | A |
| 308 | A |
| 309 | B |
| 310 | A |
| 311 | C |
| 312 | A |
| 313 | A |
| 314 | A |
| 315 | A |
| 316 | A |
| 317 | A |
| 318 | A |
| 319 | A |
| 320 | B |
| 321 | B |
| 322 | B |
| 323 | A |
| 324 | A |
| 325 | B |
| 326 | A |
| 327 | B |
| 328 | A |
| 329 | A |
| 330 | A |
| 331 | B |
| 332 | B |
| 333 | B |
| 334 | B |
| 335 | C |
| 336 | A |
| 337 | A |
| 338 | B |
| 339 | B |
| 340 | A |
| 341 | B |
| 342 | B |
| 343 | |
| 344 | D |
| 345 | |
| 346 | C |
| 347 | A |
| 348 | C |
| 349 | B |
| 350 | B |
| 351 | B |
| 352 | B |
| 353 | A |
| 354 | B |
| 355 | B |
| 356 | A |
| 357 | A |
| 358 | B |
| 359 | |
| 360 | B |
| 361 | |
| 362 | A |
| 363 | A |
| 364 | B |
| 365 | |
| 366 | |
| 367 | |
| 368 | A |
| 369 | B |
| 370 | C |
| 371 | |
| 373 | A |
| 374 | A |
| 375 | A |
| 376 | B |
| 377 | B |
| 378 | B |
| 379 | B |
| 380 | A |
| 381 | |
| 382 | A |
| 383 | A |
| 384 | D |
| 385 | D |
| 386 | C |
| 387 | A |
| 388 | A |
| 389 | B |
| 390 | A |
| 391 | A |
| 392 | A |
| 393 | B |
| 395 | B |
| 396 | B |
| 397 | B |
| 398 | C |
| 399 | C |
| 400 | |
| 403 | B |
| 404 | B |
| 405 | C |
| 406 | B |
| 407 | B |
| 408 | B |
| 409 | B |
| 410 | B |
| 411 | C |
| 412 | B |
| 413 | B |
| 414 | B |
| 415 | A |
| 416 | A |
| 417 | B |
| 418 | B |
| 419 | B |
| 420 | B |
| 421 | B |
| 422 | B |
| 424 | B |
| 425 | B |
| 432 | B |
| 433 | B |
| 434 | B |
| 435 | B |
| 436 | A |
| 437 | B |
| 438 | B |
| 439 | B |
| 440 | B |
| 441 | B |
| 444 | B |
| 445 | C |
| 448 | C |
| 449 | B |
| 450 | C |
| 453 | |
| 456 | A |
| 458 | B |
| 482 | A |
| 484 | B |
| 486 | B |
| 487 | |
| 488 | B |
| 489 | A |
| 490 | |
| 491 | A |
| 492 | C |
| 493 | A |
| 494 | |
| 495 | B |
| 496 | B |
| 497 | A |
| 498 | B |
| 499 | |
| 500 | |
| 501 | A |
| 502 | C |
| 503 | A |
| 504 | B |
| 505 | B |
| 506 | |
| 507 | A |
| 508 | |
| 509 | |
| 510 | A |
| 511 | A |
| 512 | A |
| 513 | A |
| 514 | A |
| 515 | A |
| 516 | B |
| 517 | A |
| 518 | B |
| 519 | B |
| 520 | B |
| 521 | A |
| 522 | C |
| 523 | A |
| 524 | D |
| 525 | B |
| 526 | B |
| 527 | A |
| 528 | A |
| 529 | A |
| 530 | A |
| 531 | A |
| 532 | B |
| 533 | B |
| 534 | B |
| 535 | A |
| 536 | A |
| 537 | A |
| 538 | C |
| 539 | A |
| 540 | A |
| 541 | C |
| 542 | B |
| 543 | A |
| 544 | B |
| 615 | |
| 617 | |
| 652 | A |
| 653 | A |
| 660 | A |
| 661 | B |
| 668 | A |
| 669 | A |
| 676 | A |
| 677 | B |
| 705 | A |
| 706 | A |
| 707 | A |
| 712 | A |
| 717 | A |
| 722 | A |
| 723 | A |
| 728 | A |
| 729 | A |
| 732 | B |
| 737 | A |
| 742 | A |
| 743 | C |
| 744 | A |
| 745 | A |
| 748 | B |
| 749 | B |
| 750 | B |
| 751 | B |
| 752 | B |
| 753 | A |
| 756 | B |
| 757 | A |
| 758 | B |
| 760 | B |
| 763 | A |
| 768 | C |
| 770 | B |
| 773 | B |
| 779 | A |
| 780 | B |
| 781 | C |
| 782 | B |
| 783 | B |
| 784 | A |
| 785 | A |
| 786 | B |
| 787 | B |
| 788 | B |
| 789 | B |
| 790 | C |
| 791 | B |
| 792 | B |
| 793 | B |
| 794 | B |
| 795 | C |
| 796 | A |
| 797 | A |
| 798 | C |
| 799 | B |
| 800 | A |
| 801 | B |
| 802 | B |
| 803 | A |
| 804 | B |
| 805 | B |
| 806 | B |
| 807 | B |
| 808 | C |
| 809 | C |
| 810 | B |
| 811 | B |
| 812 | B |
| 813 | B |
| 814 | B |
| 815 | B |
| 816 | B |
| 817 | B |
| 818 | A |
| 819 | B |
| 820 | C |
| 821 | C |
| 822 | B |
| 823 | B |
| 824 | B |
| 825 | C |
| 826 | B |
| 827 | B |
| 828 | B |
| 829 | B |
| 830 | B |
| 831 | B |
| 832 | C |
| 833 | C |
| 834 | C |
| 835 | B |
| 836 | C |
| 837 | C |
| 838 | C |
| 896 | B |
| 924 | C |
| 925 | C |

### Example 336: Evaluating Effect of Exemplary Compounds on Protein Abundance

Compounds described herein were used to screen for effects on quantitative protein abundance using a HiBit assay system (Promega). Quantitative protein abundance was determined by measuring the protein levels of HiBit-tagged protein targets expressed in cell culture via luminescence using the Nano-Glo HiBiT Lytic Detection System, which uses a split complementation assay format to reconstitute NanoBiT enzyme to generate a luminescent signal. A protein abundance assay was developed such that endogenous protein targets could be modified with the HiBiT peptide tag and their abundance could be assessed after compound treatment. Briefly, K562 cell lines containing a HiBiT-modification were treated with various compounds described herein (e.g., compounds of Formulas (I), or (II)). After treatment for 24 hours, the protein abundance of a specific target was determined by measuring luminescence.

### Materials:

Promega Nano-Glo HiBiT Lytic Detection System (cat #N3030)
Corning 384-well TC-treated microplates (cat #3570)

### Synthego Engineered Cells Knock-In Clones

TABLE 5
Design of genetically modified HiBiT cell lines

| | | | Guide | Guide | |
|---|---|---|---|---|---|
| Cell | | | RNA | RNA cut | |
| Line | Gene | Modification | Sequence | location | Donor Sequence |
| K562 | MYB | HiBiT | GCGCCATG | chr6:135, | CGGTGCGGTCCCCGCGGCTC |
| | | | GCCCGAAG | 181, 526 | TCGGCGGAGCCCCGCGCCCG |
| | | | ACCC | | CCGCGCCATGgtgagcggct |
| | | | | | ggcggctgttcaagaagatt |
| | | | | | agcGGCAGCTCCGGAGGATC |
| | | | | | TAGCGGCGCCCGAAGACCCC |
| | | | | | GGCACAGgtaacggggagcc |
| | | | | | ggggggggccgaggg |
| K562 | HTT | HiBiT | CAGCTTTT | chr4:3, | CGAGTCGGCCCGAGGCCTCC |
| | | | CCAGGGTC | 074, 830 | GGGGACTGCCGTGCCGGGCG |
| | | | GCCA | | GGAGACCGCCATGgtgagcg |
| | | | | | gctggcggctgttcaagaag |
| | | | | | attagcGGCAGCTCCGGAGG |
| | | | | | ATCTAGCGGCGCGACCCTGG |
| | | | | | AAAAGCTGATGAAGGCCTTC |
| | | | | | GAGTCCCTCAAGTCCTTCCA |

### Description:

Cells were maintained in IMIDM with 10% FBS. Before the assay, cells were diluted with phenolphthalein-free growth media (IMIDM+100 FBS media) and were seeded in a 384-well plate at a density of 10000 cells/well (for each cell line listed in Table 6). Each compound was prepared as a 10-point 3-fold serial dilution in DMSO with the top dose at a final concentration of 10 μM in the well. Unmodified K562 cells were added at the previously specified density with DMSO to serve as an assay baseline and positive control (PC) and DMSO only with the respective modified cell lines was added to the negative control (NC) columns. Final DMSO concentration was kept at or below 0.25%. Treated cell plates were placed in an incubator at 37° C. with 500 CO₂ for 24 hours. After 24 hours, 25 μL of Complete HiBit Lytic reagent was added to each well at room temperature (e.g. one plate requiring 10 mL Lytic Buffer, 100 μL LgBiT Protein, 200 μL Lytic Substrate), shaken for 5 minutes at 600 RPM, then left to sit for 10 minutes for signal to stabilize before reading on a Spark Cyto plate reader (Tecan) with a 500 ms measurement time.

To determine compound effects on protein abundance of each target in Table 6, the percent response for each respective cell line was calculated at each compound concentration as follows:

% response=100*(S−PC)(NC−PC)

For the normalized response at each concentration, a four-parameter logistical regression was fit to the data and the response was interpolated at the 5000 value to determine a concentration for protein abundance at 50% (IC₅₀) the untreated control.

A summary of the results for protein abundance is illustrated in Table 6, wherein A represents <100 nM; B represents 100-1000 nM; C represents 1000-9999 nM; and D represents greater than 10 μM.

TABLE 6
Effect of Exemplary Compounds on Protein Abundance

| Cmpd No. | MYB | Target C | HTT |
|---|---|---|---|
| 100 | A | A | A |
| 101 | A | A | A |
| 102 | A | A | A |
| 104 | C | C | C |
| 106 | C | C | C |
| 107 | B | B | B |
| 108 | B | B | B |
| 109 | A | A | A |
| 110 | D | D | D |
| 111 | D | D | D |
| 112 | D | D | D |
| 115 | A | B | B |
| 116 | C | C | C |
| 117 | B | C | B |
| 118 | B | D | C |
| 119 | B | B | B |
| 120 | C | C | C |
| 121 | C | C | C |
| 124 | B | A | A |
| 125 | B | B | B |
| 126 | B | B | B |
| 127 | A | A | A |
| 129 | B | B | B |
| 131 | C | C | C |
| 132 | D | D | D |
| 133 | C | C | C |
| 135 | B | C | C |
| 136 | B | B | B |
| 137 | B | B | B |
| 138 | B | B | B |
| 139 | B | A | B |
| 140 | B | B | B |
| 141 | C | C | C |
| 142 | B | B | B |
| 143 | D | C | D |
| 144 | D | D | D |
| 145 | D | D | D |
| 146 | C | C | C |
| 148 | C | C | C |
| 149 | C | D | C |
| 150 | A | A | A |
| 151 | B | B | B |
| 152 | D | D | D |
| 153 | A | A | A |
| 154 | A | A | A |
| 155 | A | A | A |
| 156 | A | A | A |
| 157 | A | B | A |
| 158 | A | B | A |
| 159 | B | C | B |
| 160 | B | B | B |
| 161 | D | D | D |
| 162 | D | D | D |
| 163 | C | D | C |
| 164 | B | B | B |
| 165 | C | C | C |
| 166 | D | C | D |
| 167 | D | D | D |
| 168 | D | D | D |
| 169 | C | D | C |
| 170 | B | B | A |
| 171 | A | A | A |
| 172 | A | B | A |
| 173 | A | A | A |
| 174 | D | D | D |
| 175 | A | A | A |
| 176 | C | C | C |
| 177 | C | C | C |
| 178 | B | B | B |
| 179 | B | B | A |
| 180 | A | B | A |
| 182 | D | D | D |
| 184 | B | B | B |
| 185 | C | D | C |
| 186 | B | A | A |
| 187 | B | A | A |
| 188 | C | C | B |
| 189 | C | C | C |
| 190 | D | D | D |
| 191 | A | A | A |
| 193 | C | C | C |
| 194 | A | A | A |
| 195 | A | B | B |
| 196 | D | D | D |
| 197 | B | B | A |
| 198 | A | A | A |
| 199 | B | B | B |
| 200 | C | C | C |
| 201 | B | B | A |
| 202 | A | A | A |
| 203 | A | A | A |
| 204 | C | C | C |
| 205 | C | C | C |
| 206 | C | C | C |
| 207 | C | C | C |
| 208 | D | D | C |
| 209 | C | A | A |
| 210 | A | A | A |
| 211 | A | A | A |
| 212 | B | B | A |
| 214 | A | A | A |
| 215 | A | A | A |
| 216 | A | A | A |
| 217 | A | A | A |
| 218 | C | D | D |
| 219 | A | A | A |
| 220 | A | A | A |
| 221 | A | A | A |
| 222 | A | A | A |
| 223 | A | A | A |
| 224 | A | A | A |
| 225 | A | A | A |
| 226 | A | A | A |
| 228 | A | B | A |
| 229 | A | A | A |
| 230 | B | A | A |
| 231 | A | A | A |
| 232 | A | A | A |
| 233 | A | A | A |
| 234 | A | A | A |
| 235 | A | A | A |
| 236 | A | A | A |
| 237 | A | A | A |
| 238 | A | A | A |
| 241 | A | A | A |
| 245 | D | D | C |
| 246 | A | A | A |
| 247 | B | B | B |
| 248 | C | B | B |
| 249 | A | A | A |
| 250 | A | A | A |
| 251 | D | B | D |
| 252 | B | B | B |
| 253 | A | A | A |
| 254 | A | A | A |
| 255 | A | A | A |
| 256 | A | A | A |
| 257 | A | A | A |
| 258 | B | B | B |
| 259 | A | A | A |
| 260 | A | A | A |
| 284 | A | A | A |
| 285 | A | A | A |
| 286 | A | A | A |
| 287 | A | A | A |
| 288 | A | A | A |
| 289 | A | A | A |
| 290 | A | A | A |
| 291 | B | B | A |
| 292 | B | A | A |
| 293 | A | A | A |
| 294 | A | A | A |
| 295 | A | A | A |
| 296 | A | A | A |
| 297 | C | C | C |
| 298 | A | A | A |
| 299 | A | B | A |
| 300 | A | A | A |
| 302 | B | B | B |
| 303 | A | A | A |
| 304 | A | A | A |
| 305 | A | A | A |
| 306 | A | A | A |
| 307 | A | A | A |
| 308 | A | A | A |
| 309 | A | A | A |
| 310 | A | A | A |
| 311 | B | A | A |
| 312 | A | A | A |
| 313 | A | A | A |
| 314 | A | A | A |
| 315 | A | A | A |
| 316 | A | A | A |
| 317 | A | A | A |
| 318 | A | A | A |
| 319 | A | A | A |
| 320 | A | A | A |
| 321 | A | A | A |
| 322 | A | A | A |
| 323 | A | A | A |
| 324 | A | A | A |
| 325 | A | A | A |
| 326 | A | A | A |
| 327 | A | A | A |
| 328 | A | A | A |
| 329 | A | A | A |
| 330 | A | A | A |
| 331 | B | B | B |
| 332 | B | B | B |
| 333 | B | B | B |
| 334 | B | B | B |
| 335 | C | C | C |
| 336 | A | A | A |
| 337 | A | B | B |
| 338 | A | B | B |
| 339 | B | B | B |
| 340 | B | B | B |
| 341 | B | A | B |
| 342 | B | B | B |
| 344 | C | C | C |
| 346 | C | C | C |
| 347 | A | A | A |
| 348 | A | B | A |
| 349 | A | A | A |
| 350 | B | B | A |
| 351 | A | A | A |
| 352 | A | A | A |
| 353 | A | A | A |
| 354 | A | A | A |
| 355 | A | A | A |
| 356 | A | A | A |
| 357 | A | A | A |
| 358 | A | A | A |
| 359 | | | |
| 360 | A | A | A |
| 361 | | | |
| 362 | A | A | A |
| 363 | A | A | A |
| 364 | C | C | D |
| 368 | A | B | B |
| 369 | A | B | B |
| 370 | B | B | B |
| 373 | A | A | A |
| 374 | A | A | A |
| 375 | A | A | A |
| 376 | A | A | A |
| 377 | A | A | A |
| 378 | A | A | A |
| 379 | A | B | A |
| 380 | A | B | A |
| 382 | A | C | B |
| 383 | A | A | A |
| 384 | C | C | C |
| 385 | B | B | A |
| 386 | B | C | C |
| 387 | A | A | A |
| 388 | A | A | A |
| 389 | A | A | A |
| 390 | A | A | A |
| 391 | A | A | A |
| 392 | A | A | A |
| 393 | A | B | B |
| 395 | B | B | B |
| 396 | B | A | A |
| 397 | B | C | C |
| 398 | B | C | C |
| 399 | C | D | D |
| 403 | A | B | B |
| 404 | B | A | A |
| 405 | C | C | C |
| 406 | B | C | C |
| 407 | B | C | B |
| 408 | B | B | B |
| 409 | B | B | B |
| 410 | B | B | B |
| 411 | B | C | C |
| 412 | B | B | B |
| 413 | A | A | A |
| 414 | C | B | B |
| 415 | A | A | A |
| 416 | A | A | A |
| 417 | A | C | B |
| 418 | B | B | B |
| 419 | B | B | B |
| 420 | B | B | B |
| 421 | B | B | B |
| 422 | B | B | B |
| 424 | C | B | C |
| 425 | C | C | C |
| 432 | B | A | A |
| 433 | B | A | A |
| 434 | B | A | B |
| 435 | B | B | B |
| 436 | A | A | A |
| 437 | B | A | A |
| 438 | B | A | A |
| 439 | B | A | A |
| 440 | B | A | A |
| 441 | C | B | B |
| 444 | A | B | A |
| 445 | B | C | B |
| 448 | B | B | B |
| 449 | B | B | B |
| 450 | C | C | C |
| 456 | A | A | A |
| 458 | B | C | C |
| 482 | A | B | B |
| 484 | B | C | C |
| 486 | B | C | C |
| 488 | B | A | A |
| 489 | A | A | A |
| 491 | A | A | A |
| 492 | B | C | B |
| 493 | A | A | A |
| 495 | A | A | A |
| 496 | A | A | A |
| 497 | A | A | A |
| 498 | A | A | A |
| 501 | A | B | B |
| 502 | C | C | C |
| 503 | A | B | A |
| 504 | B | B | B |
| 505 | A | B | B |
| 506 | A | B | B |
| 507 | A | C | B |
| 508 | C | D | D |
| 509 | C | D | D |
| 510 | A | B | A |
| 511 | A | C | B |
| 512 | A | B | B |
| 513 | A | C | B |
| 514 | A | B | B |
| 515 | A | A | A |
| 516 | A | B | A |
| 517 | A | B | A |
| 518 | A | C | C |
| 519 | B | C | C |
| 520 | A | B | B |
| 521 | A | B | A |
| 522 | B | C | C |
| 523 | A | C | B |
| 524 | D | D | D |
| 525 | A | B | B |
| 526 | B | B | B |
| 527 | A | B | A |
| 528 | A | B | B |
| 529 | A | B | A |
| 530 | A | B | B |
| 531 | A | C | B |
| 532 | A | B | B |
| 533 | B | C | C |
| 534 | B | C | C |
| 535 | A | B | A |
| 536 | A | A | A |
| 537 | A | A | A |
| 538 | C | C | C |
| 539 | A | A | A |
| 540 | A | B | B |
| 541 | D | D | D |
| 542 | A | B | C |
| 543 | A | B | A |
| 544 | B | B | B |
| 652 | A | B | A |
| 653 | A | B | A |
| 660 | A | B | B |
| 661 | A | B | B |
| 668 | A | B | B |
| 669 | A | B | B |
| 676 | A | B | B |
| 677 | A | B | B |
| 705 | A | B | B |
| 706 | A | B | B |
| 707 | A | B | B |
| 712 | A | C | B |
| 717 | A | B | B |
| 722 | A | B | A |
| 723 | A | A | A |
| 728 | A | B | B |
| 729 | A | A | A |
| 732 | C | C | C |
| 737 | B | C | C |
| 742 | B | D | C |
| 743 | C | C | C |
| 744 | A | A | A |
| 745 | A | B | B |
| 748 | B | D | D |
| 749 | B | D | D |
| 750 | B | B | B |
| 751 | B | D | D |
| 752 | A | B | B |
| 753 | B | B | B |
| 756 | B | B | B |
| 757 | A | C | C |
| 758 | B | B | B |
| 760 | A | B | B |
| 763 | A | A | A |
| 768 | A | C | C |
| 770 | A | B | A |
| 773 | A | B | B |
| 779 | A | B | B |
| 796 | A | B | B |
| 797 | A | C | B |
| 799 | B | B | B |
| 800 | A | A | A |
| 803 | A | B | B |
| 805 | A | B | A |
| 806 | A | C | B |
| 807 | A | D | B |

### EQUIVALENTS AND SCOPE

This application refers to various issued patents, published patent applications, journal articles, and other publications, all of which are incorporated herein by reference. If there is a conflict between any of the incorporated references and the instant specification, the specification shall control. In addition, any particular embodiment of the present invention that falls within the prior art may be explicitly excluded from any one or more of the claims. Because such embodiments are deemed to be known to one of ordinary skill in the art, they may be excluded even if the exclusion is not set forth explicitly herein. Any particular embodiment of the invention can be excluded from any claim, for any reason, whether or not related to the existence of prior art.

Those skilled in the art will recognize or be able to ascertain using no more than routine experimentation many equivalents to the specific embodiments described herein. The scope of the present embodiments described herein is not intended to be limited to the above Description, Figures, or Examples but rather is as set forth in the appended claims. Those of ordinary skill in the art will appreciate that various changes and modifications to this description may be made without departing from the spirit or scope of the present invention, as defined in the following claims.

1. A compound of Formula (I): 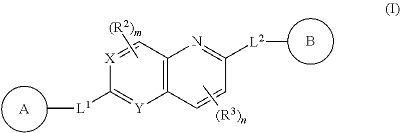
or a pharmaceutically acceptable salt, solvate, hydrate, tautomer, or stereoisomer thereof, wherein:
A and B are each independently heterocyclyl or heteroaryl, wherein each heterocyclyl and heteroaryl is optionally substituted with one or more R¹;
L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵;
X and Y are each N or C(R⁶);
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, heteroaryl, aryl, C₁-C₆ alkylene-aryl, C₂-C₆ alkenylene-aryl, C₁-C₆ alkylene-heteroaryl, C₂-C₆ alkenylene-heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkenylene, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
each R² and R³ is independently C₁-C₆-alkyl, C₂-C₆-alkenyl, C₂-C₆-alkynyl, C₁-C₆-heteroalkyl, C₁-C₆-haloalkyl, halo, cyano, cycloalkyl, heterocyclyl, aryl, heteroaryl, —OR^{A}, —NR^{B}R^{C}, NR^{B}C(O)R^{D}, —NO₂, —C(O)NR^{B}R^{C}, —C(O)R^{D}, —C(O)OR^{D}, or —S(O)ₓR^{D};
each R⁴ is independently hydrogen, C₁-C₆-alkyl, or C₁-C₆-haloalkyl;
each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C};
each R⁶ is independently hydrogen, halo, cyano, C₁-C₆-alkyl, C₁-C₆-haloalkyl, or —OR^{A};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸;
each R^{A} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-aryl, C₁-C₆ alkylene-heteroaryl, —C(O)R^{D}, or —S(O)ₓR^{D}, wherein each alkyl, alkylene, heteroalkyl, haloalkyl, cycloalkyl, heterocyclyl, aryl, and heteroaryl is optionally substituted with one or more R⁹;
each R^{B} and R^{C} is independently hydrogen, C₁-C₆ alkyl, C₁-C₆ heteroalkyl, C₁-C₆ haloalkyl, cycloalkyl, heterocyclyl, aryl, heteroaryl, C₁-C₆ alkylene-cycloalkyl, C₁-C₆ alkylene-heterocyclyl, —OR^{A}, wherein each alkyl, alkylene, heteroalkyl, haloalkyl, cycloalkyl, heterocyclyl, aryl, and heteroaryl is optionally substituted with one or more R⁹; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, wherein each alkyl, alkenyl, alkynyl, heteroalkyl, and haloalkyl is optionally substituted with one or more R⁹;
each R⁸ is independently C₁-C₆ alkyl, C₁-C₆ heteroalkyl, C₁-C₆ haloalkyl, halo, cyano, oxo, or —OR^{A};
each R⁹ is C₁-C₆-alkyl, C₁-C₆-alkenyl, C₁-C₆-alkynyl, C₁-C₆-heteroalkyl, C₁-C₆-haloalkyl, halo, cyano, oxo, or —OR^{A1};
each R^{A1} is hydrogen or C₁-C₆-alkyl;
n is 0, 1, or 2;
m is 0 or 1; and
x is 0, 1, or 2.

2. The compound of claim 1, wherein one of A and B is independently a monocyclic heteroaryl or bicyclic heteroaryl, each of which is optionally substituted with one or more R¹.

3. The compound of any one of the preceding claims, wherein one of A and B is independently a bicyclic heteroaryl optionally substituted with one or more R¹.

4. The compound of any one of the preceding claims, wherein one of A and B is independently a nitrogen-containing heteroaryl optionally substituted with one or more R¹.

5. The compound of any one of the preceding claims, wherein one of A and B is a 5-10 membered heteroaryl optionally substituted with one or more R¹.

6. The compound of any one of the preceding claims, wherein one of A and B is independently selected from 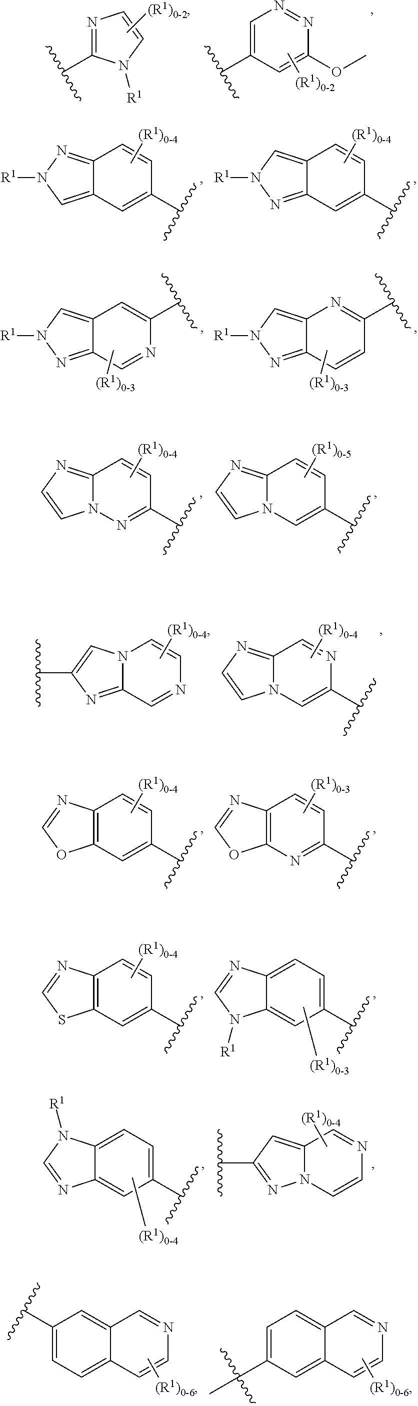 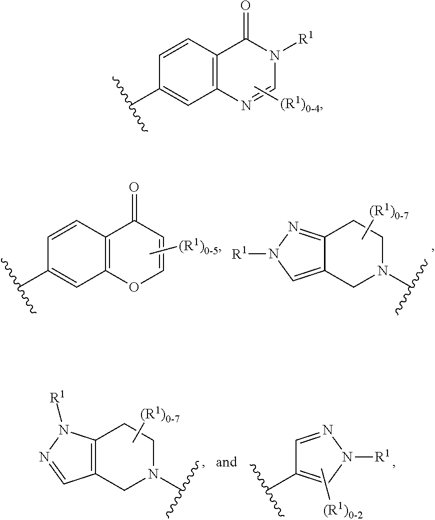
wherein R¹ is as described in claim 1.

7. The compound of any one of the preceding claims, wherein one of A and B is independently selected from 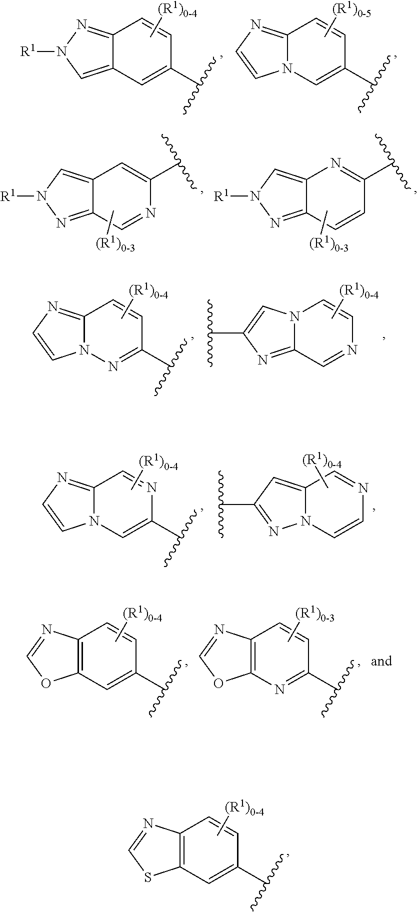
wherein R¹ is as described in claim 1.

8. The compound of any one of the preceding claims, wherein one of A and B is independently selected from 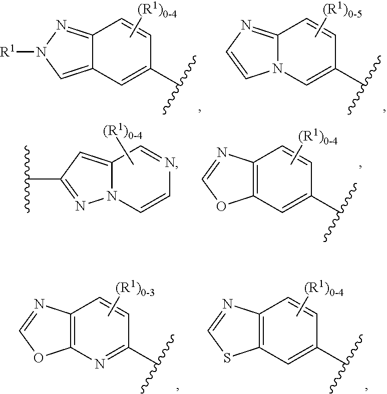
wherein R¹ is as described in claim 1.

9. The compound of any one of the preceding claims, wherein one of A and B is independently selected from 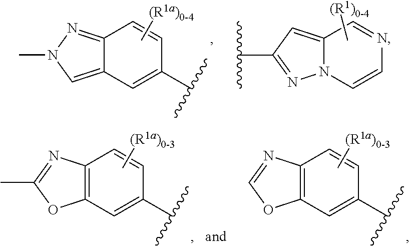
wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

10. The compound of any one of the preceding claims, wherein one of A and B is independently selected from 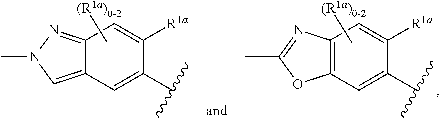
and wherein each R^{1a} is independently C₁-C₆-alkyl, C₁-C₆-heteroalkyl, C₁-C₆-haloalkyl, halo, cyano, or —OR^{A}, and each alkyl, heteroalkyl, and haloalkyl is optionally substituted with one or more R⁷.

11. The compound of claim 10, wherein at least one of R^{1a} is C₁-C₆-alkyl, halo, or —OR^{A}.

12. The compound of any one of claims 10-11, wherein R^{1a} is —OR^{A} and R^{A} is H.

13. The compound of any one of the preceding claims, wherein A is independently selected from 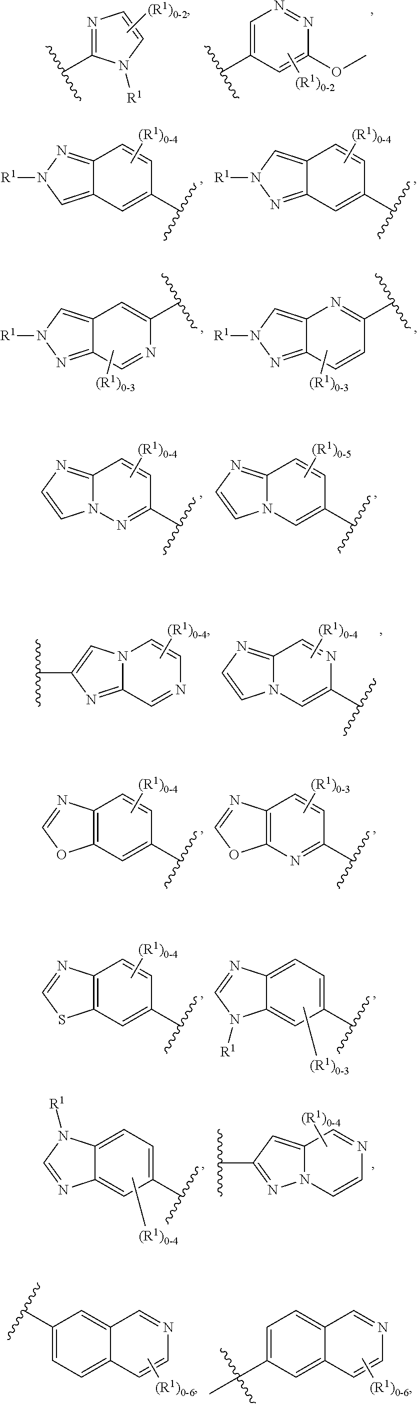 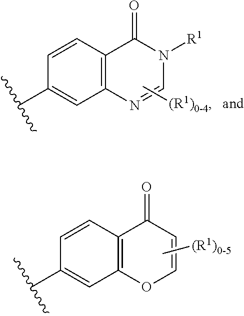
wherein R¹ is as described in claim 1.

14. The compound of any one of the preceding claims, wherein B is independently selected from 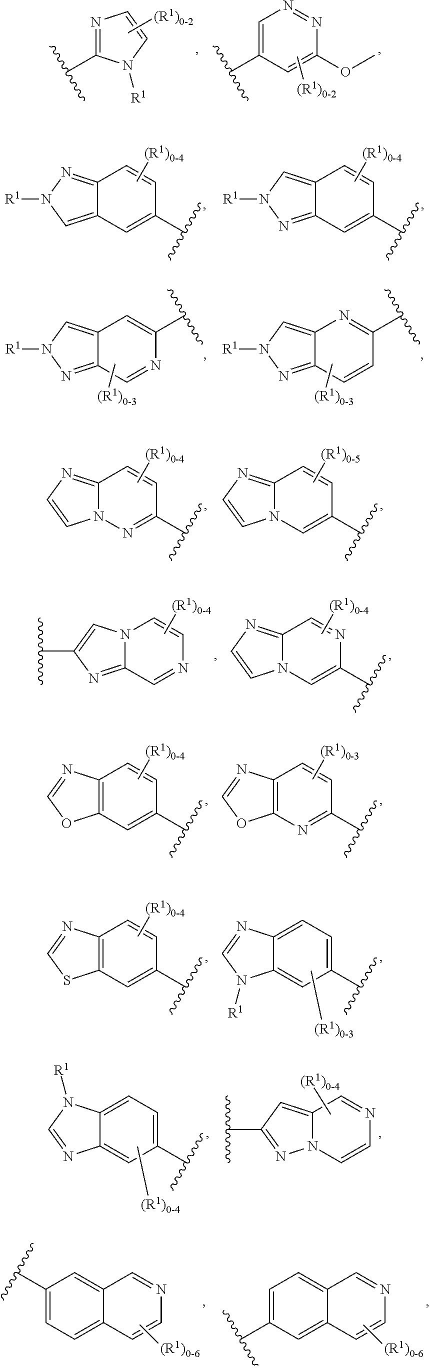 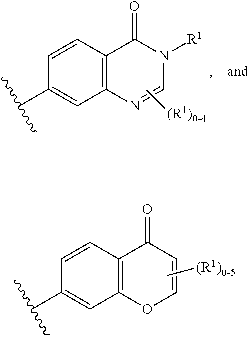
wherein R¹ is as described in claim 1.

15. The compound of any one of the preceding claims, wherein one of A and B is independently selected from

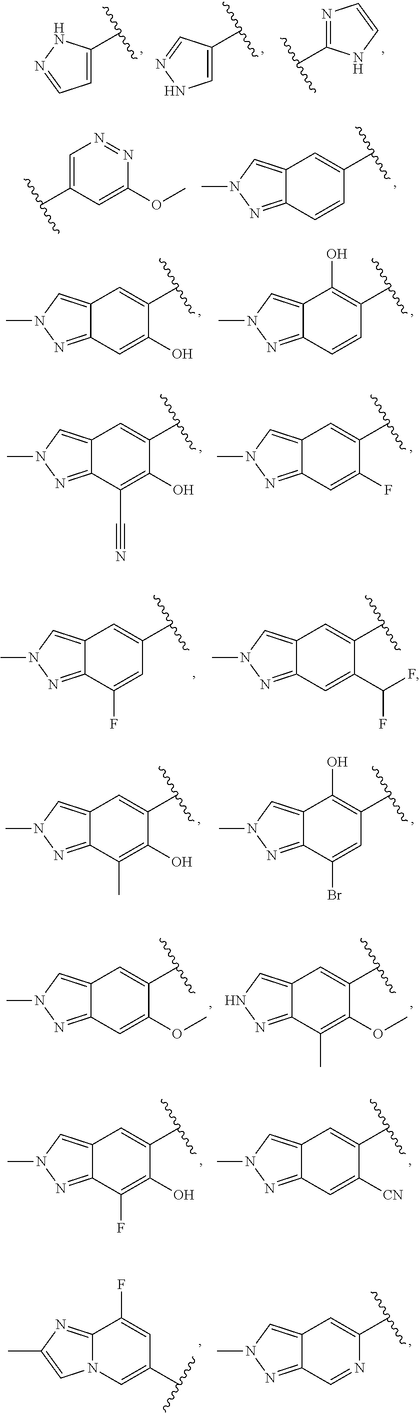

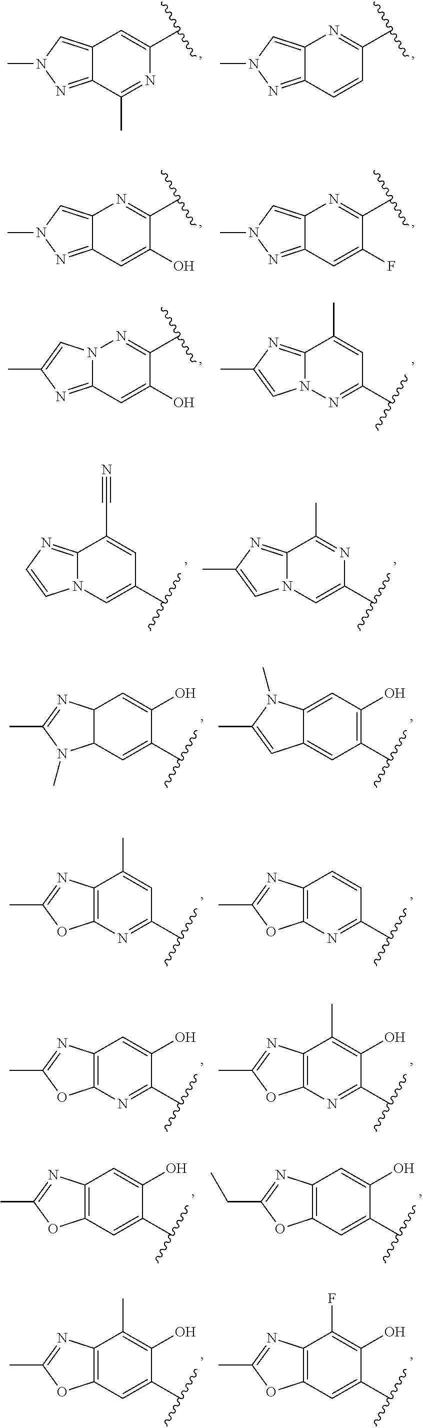

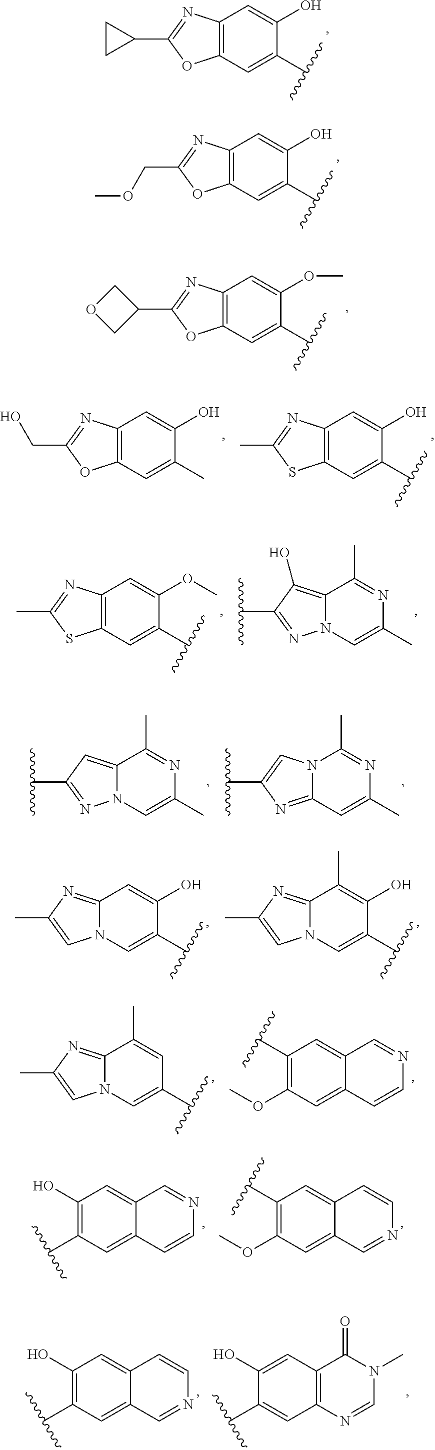

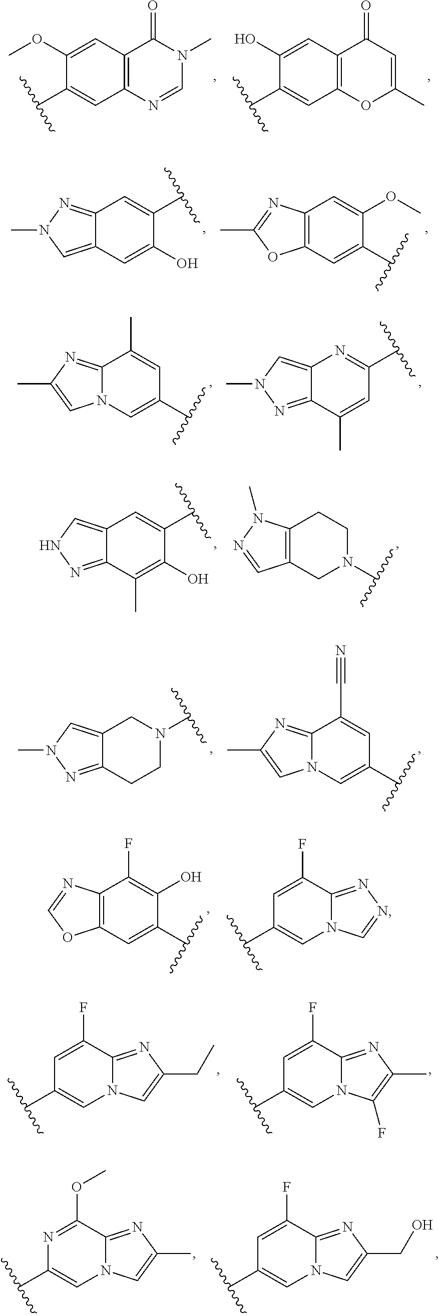

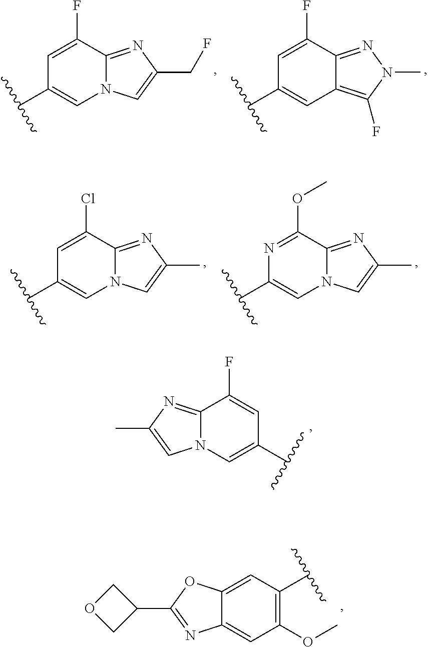 [SMILES: *c1cc(F)c2nc(CF)cn2c1 ; *c1cc(F)c2nn(C)c(F)c2c1 ; *c1cc(Cl)c2nc(C)cn2c1 ; *c1cn2cc(C)nc2c(OC)n1 ; *c1cc(F)c2nc(C)cn2c1 ; *c1cc2oc(C3COC3)nc2cc1OC]

16. The compound of any one of the preceding claims, wherein one of A and B is independently selected from

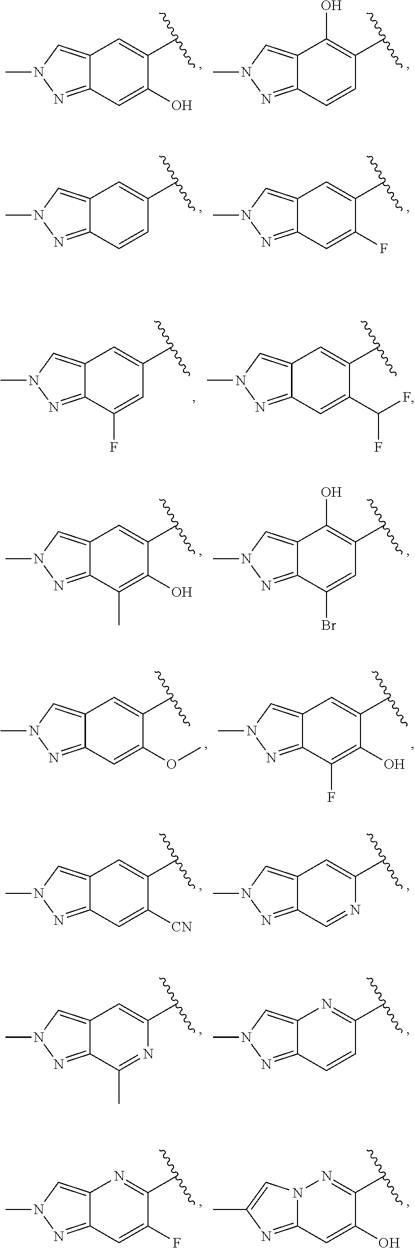

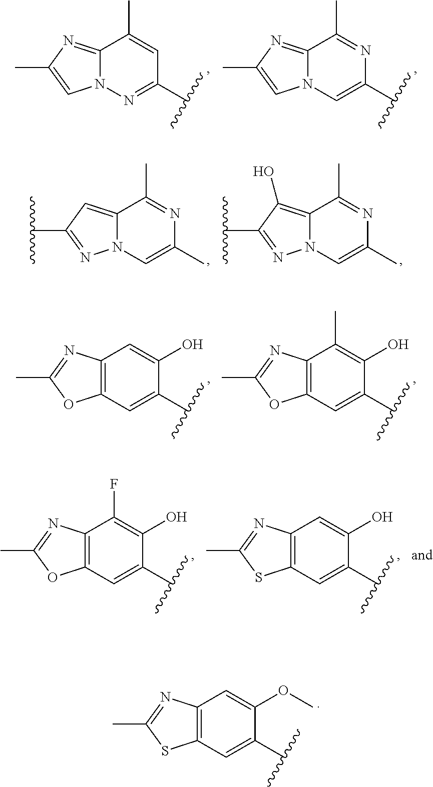

17. The compound of any one of the preceding claims, wherein one of A and B is independently selected from

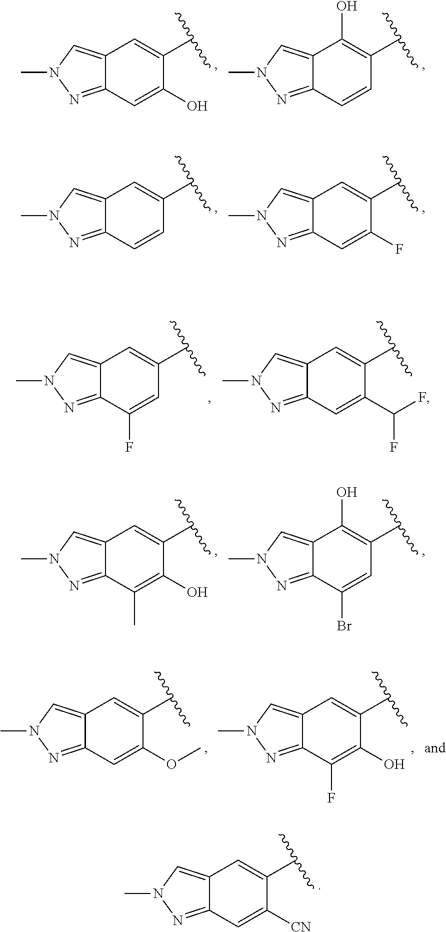

18. The compound of any one of the preceding claims, wherein one of A and B is independently selected from

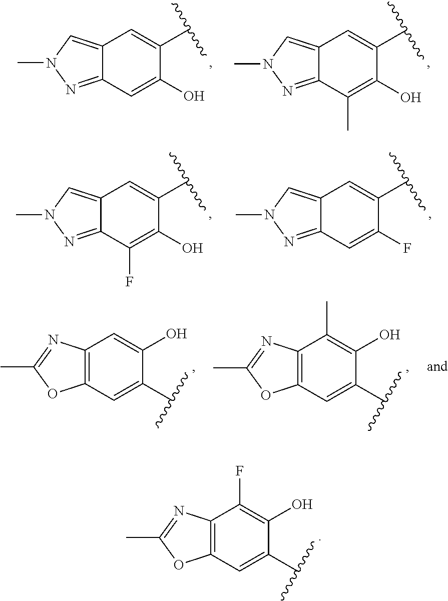

19. The compound of any one of the preceding claims, wherein A is selected from

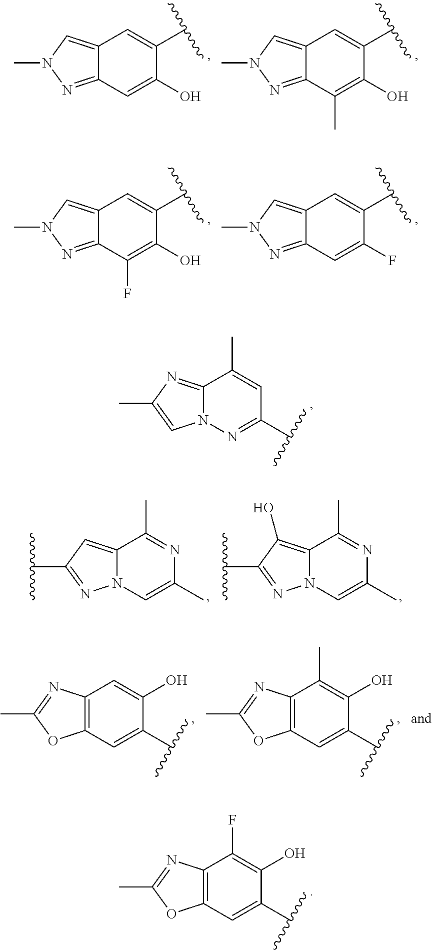

20. The compound of any one of the preceding claims, wherein B is selected from

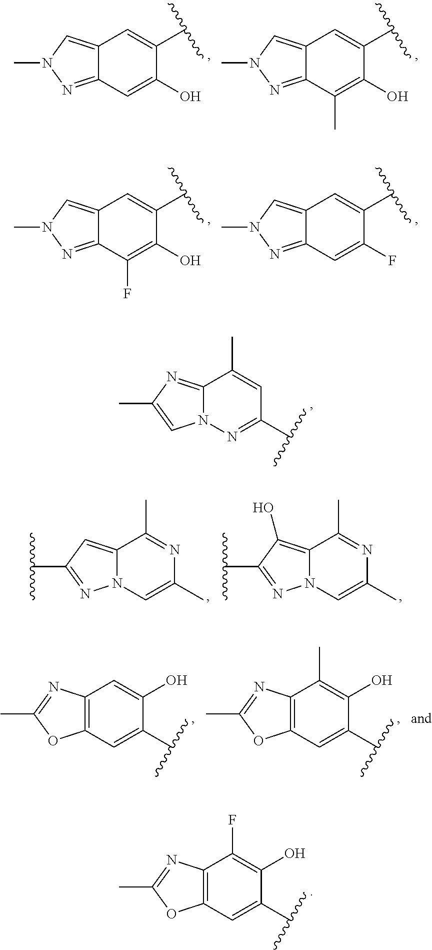

21. The compound of any one of the preceding claims, wherein one of A and B is independently a monocyclic heterocyclyl or bicyclic heterocyclyl, each of which is optionally substituted with one or more R¹.

22. The compound of any one of the preceding claims, wherein one of A and B is independently a nitrogen-containing heterocyclyl optionally substituted with one or more R¹.

23. The compound of any one of the preceding claims, wherein one of A and B is independently a 4-8 membered heterocyclyl optionally substituted with one or more R¹.

24. The compound of any one of the preceding claims, wherein one of A and B is independently selected from 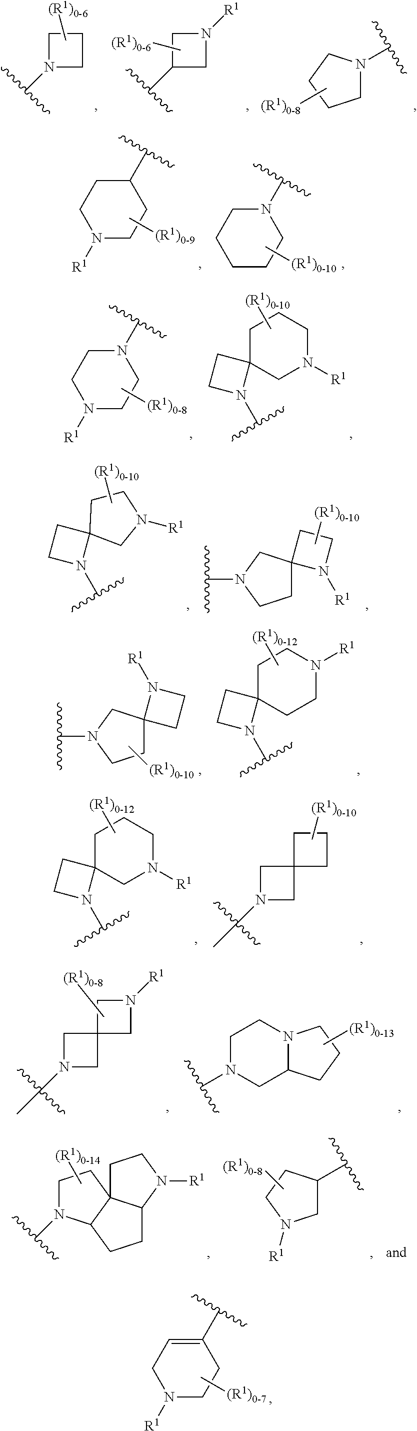
wherein R¹ is as described in claim 1.

25. The compound of any one of the preceding claims, wherein one of A and B is independently selected from 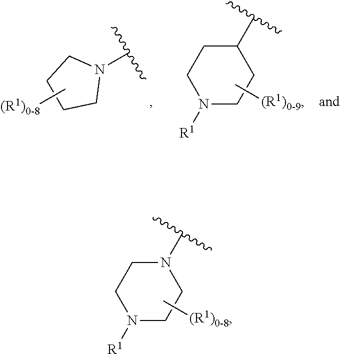
wherein R¹ is as described in claim 1.

26. The compound of any one of the preceding claims, wherein one of A and B is independently 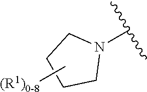
and R¹ is as described in claim 1.

27. The compound of any one of the preceding claims, wherein one of A and B is independently 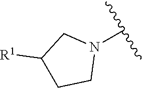
and R¹ is as described in claim 1.

28. The compound of any one of the preceding claims, wherein one of A and B is independently selected from 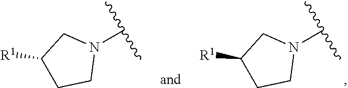
and R¹ is as described in claim 1.

29. The compound of any one of the preceding claims, wherein one of A and B is independently 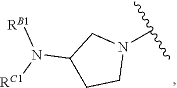
and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

30. The compound of claim 29, wherein R^{B1} is hydrogen and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

31. The compound of any one of the preceding claims, wherein one of A and B is independently selected from 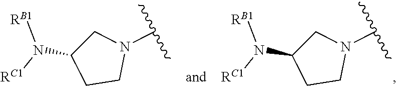
and each of R^{B1} and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

32. The compound of claim 31, wherein R^{B1} is hydrogen and R^{C1} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

33. The compound of any one of the preceding claims, wherein A is selected from 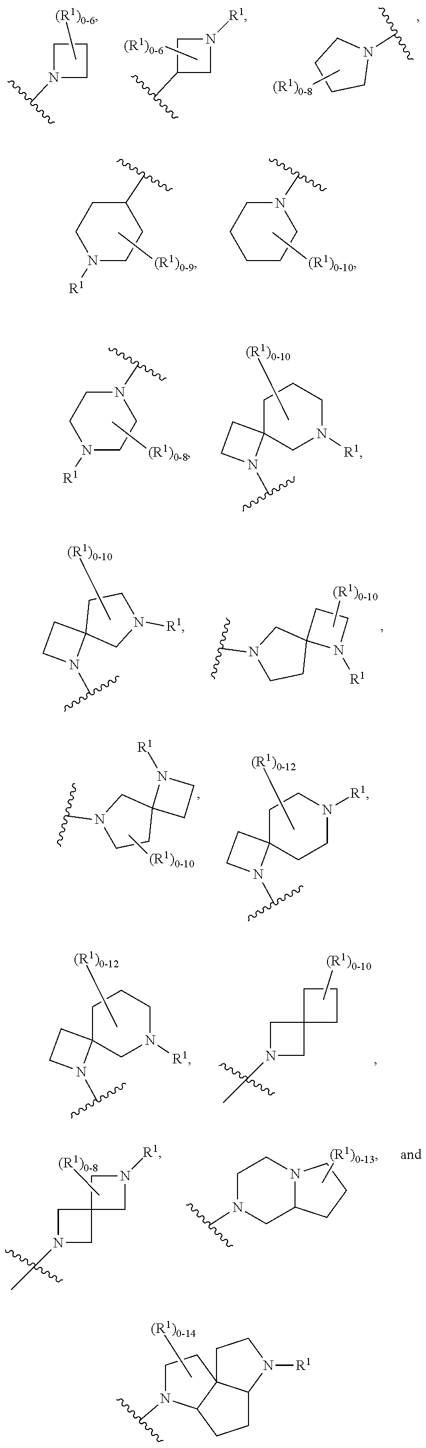
wherein R¹ is as described in claim 1.

34. The compound of any one of the preceding claims, wherein B is selected from 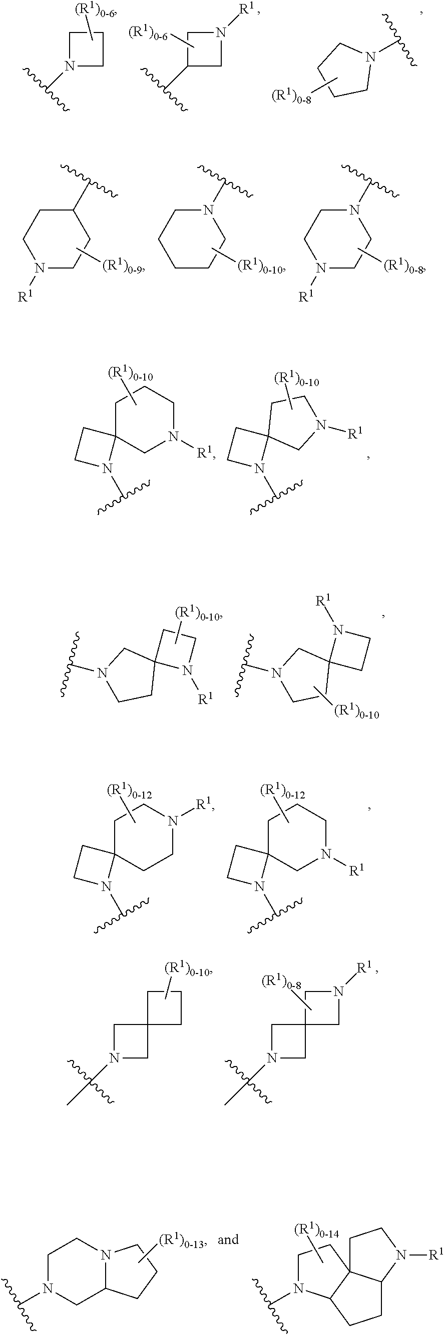
wherein R¹ is as described in claim 1.

35. The compound of any one of the preceding claims, wherein one of A and B is independently is selected from

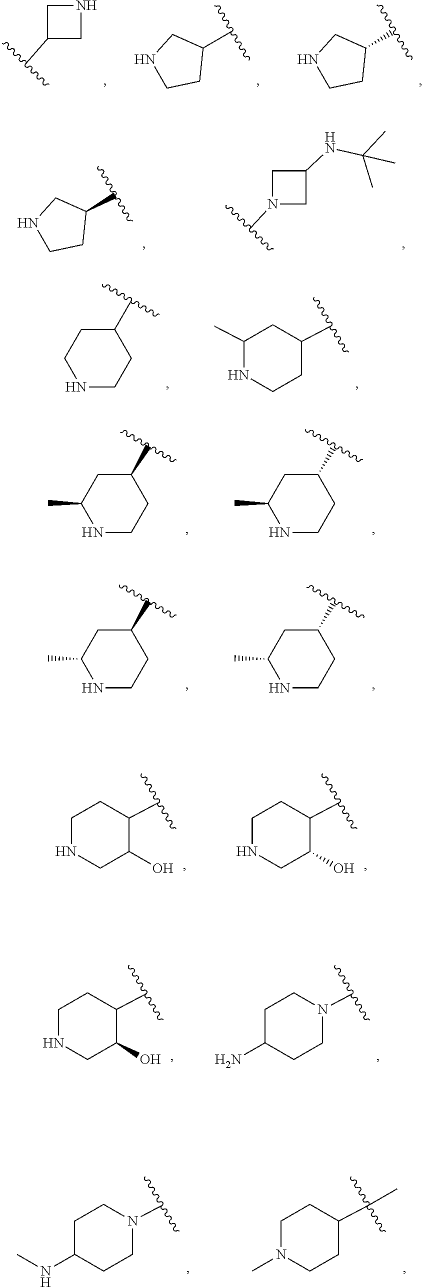

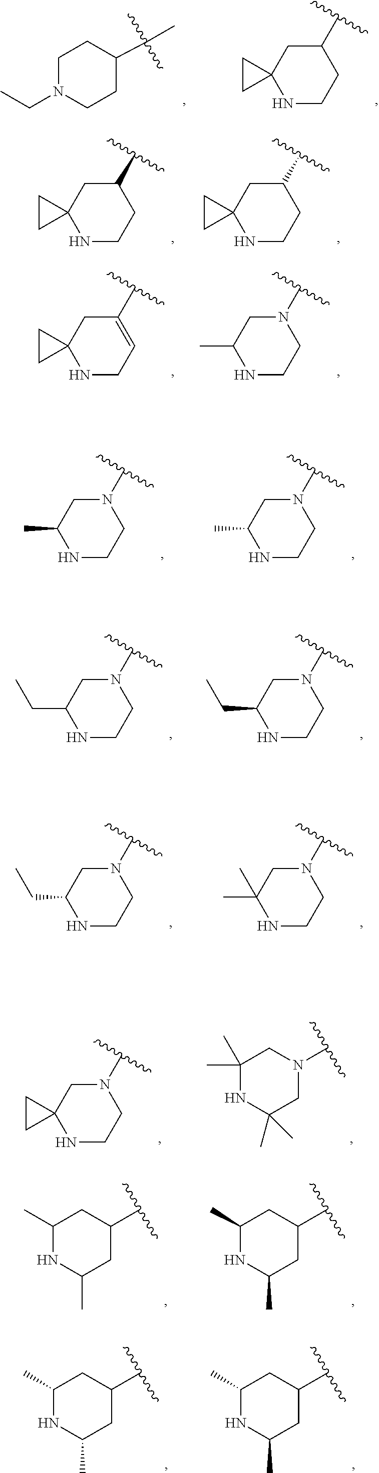

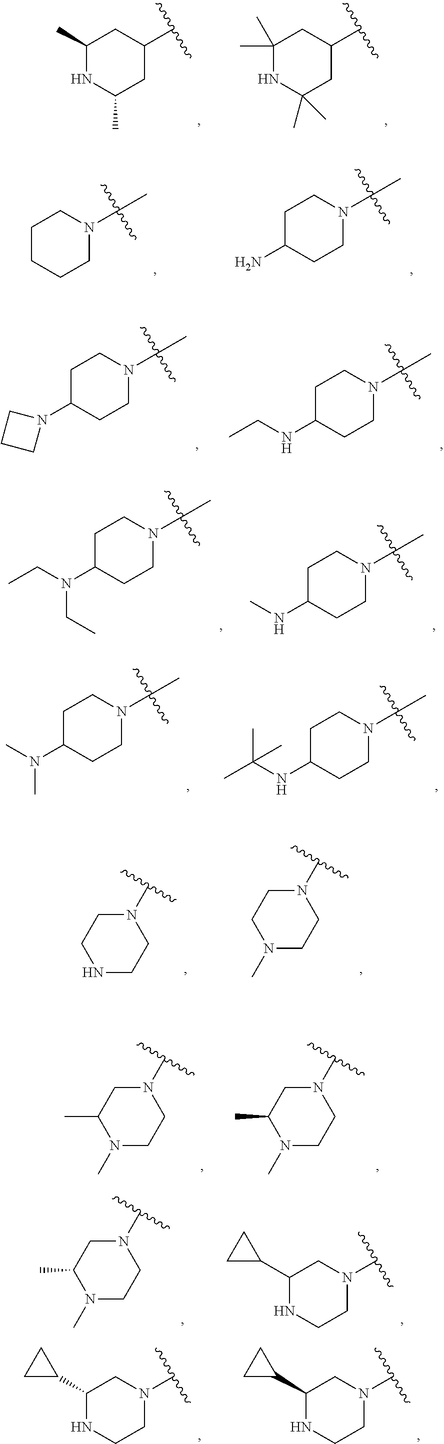

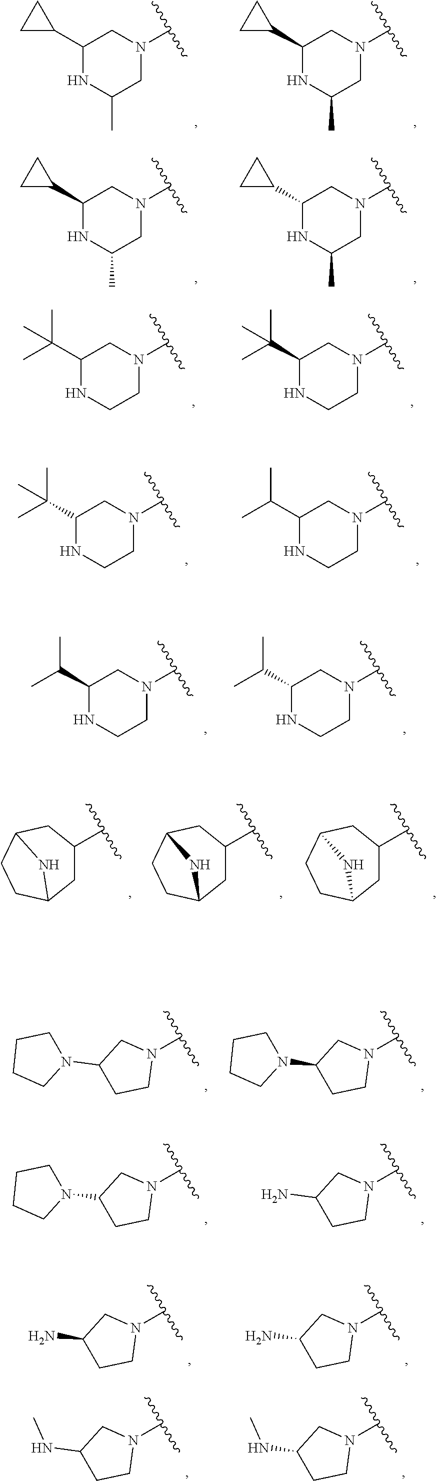

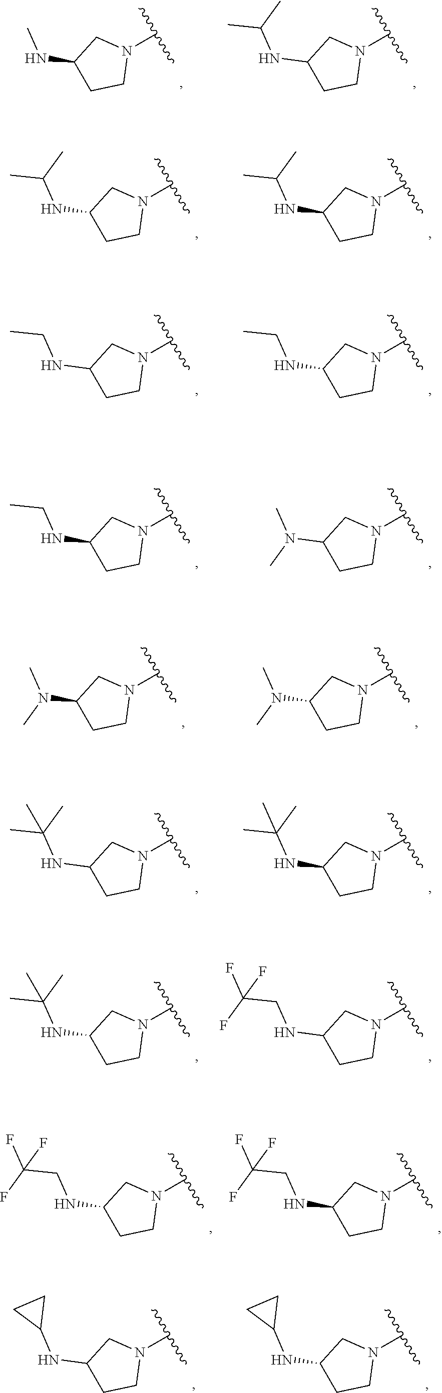

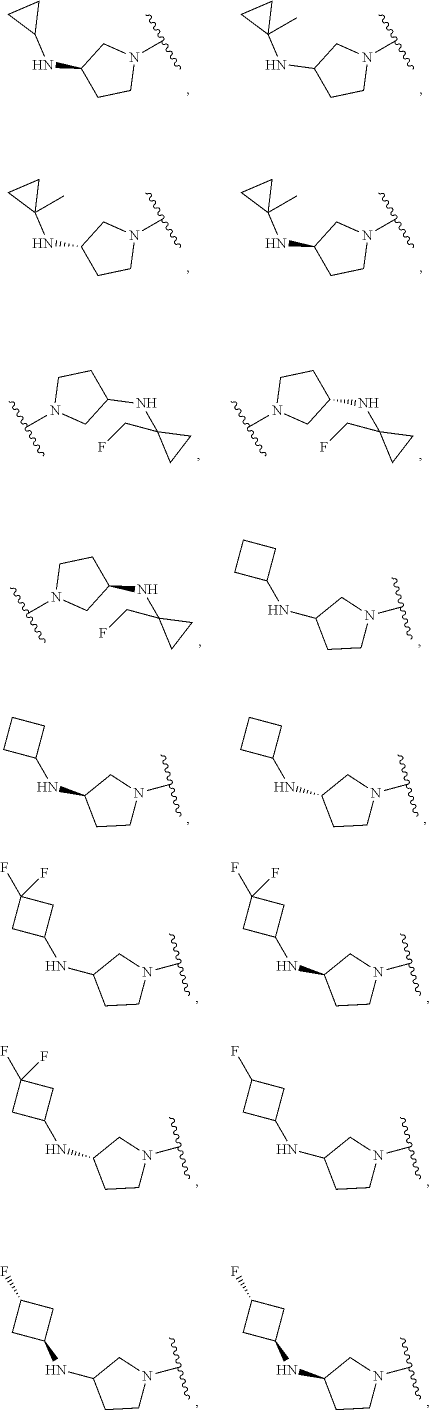

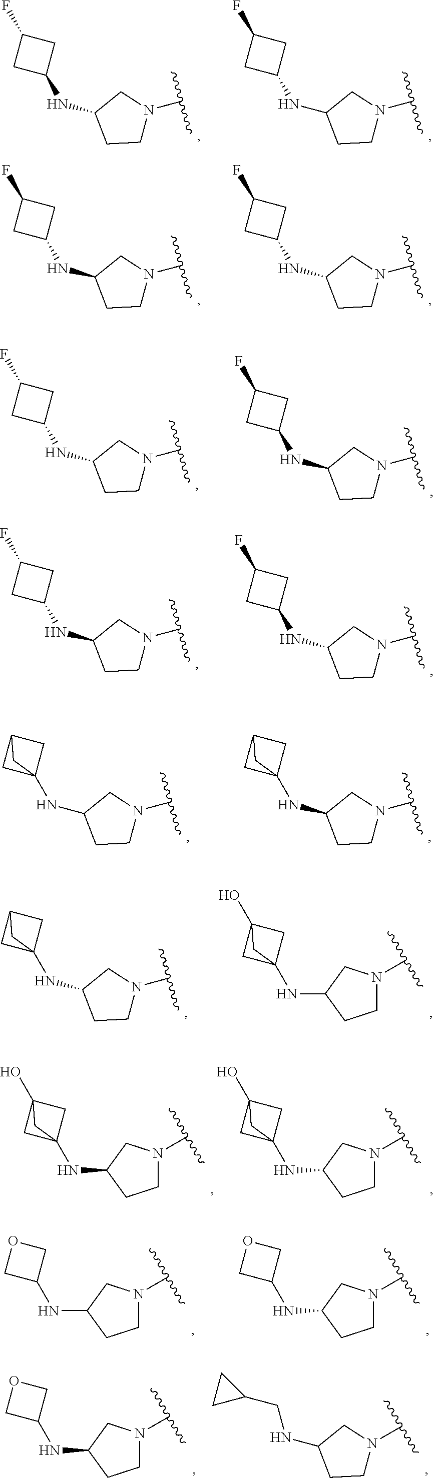

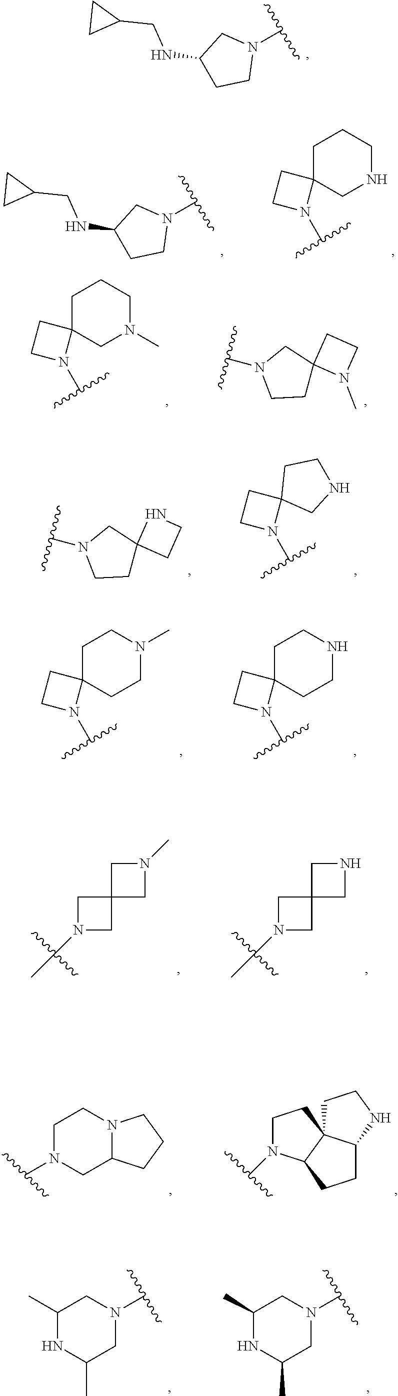

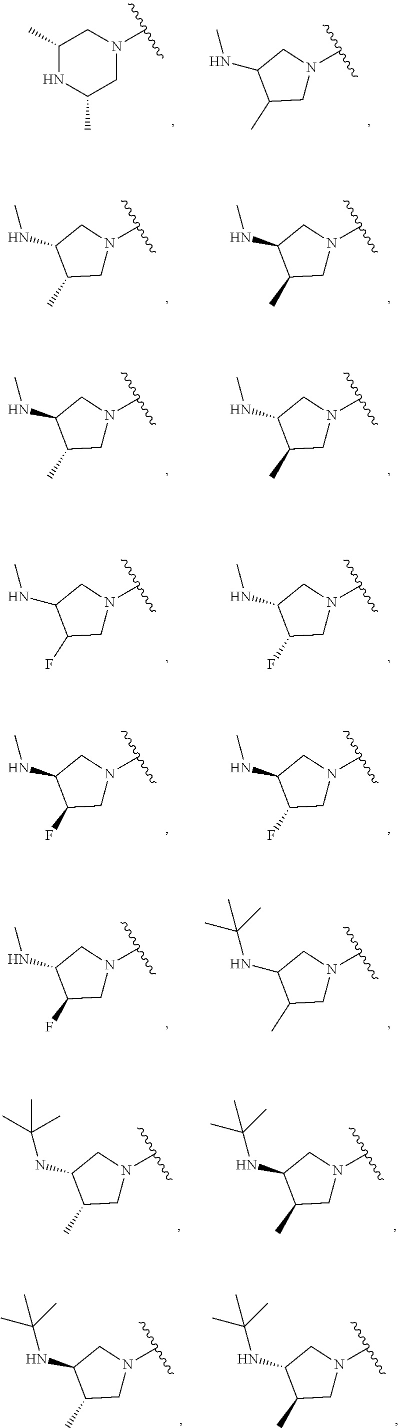

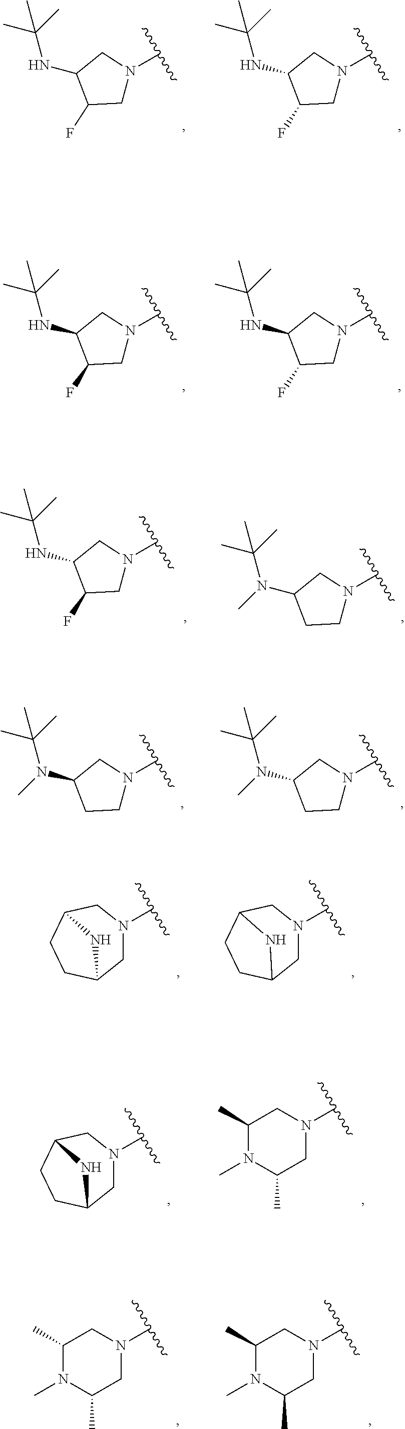

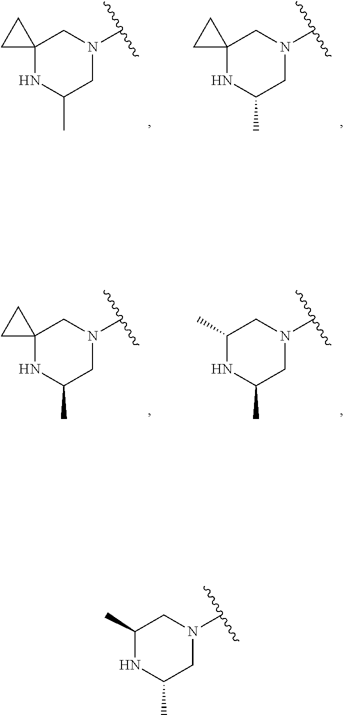

36. The compound of any one of the preceding claims, wherein one of A and B is independently is selected from

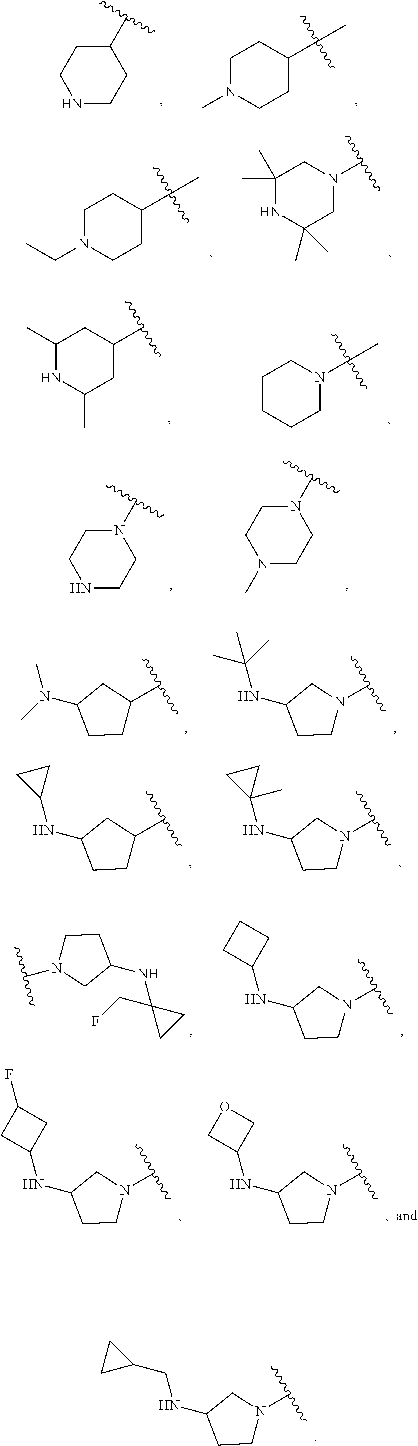

37. The compound of any one of the preceding claims, wherein one of A and B is independently is selected from

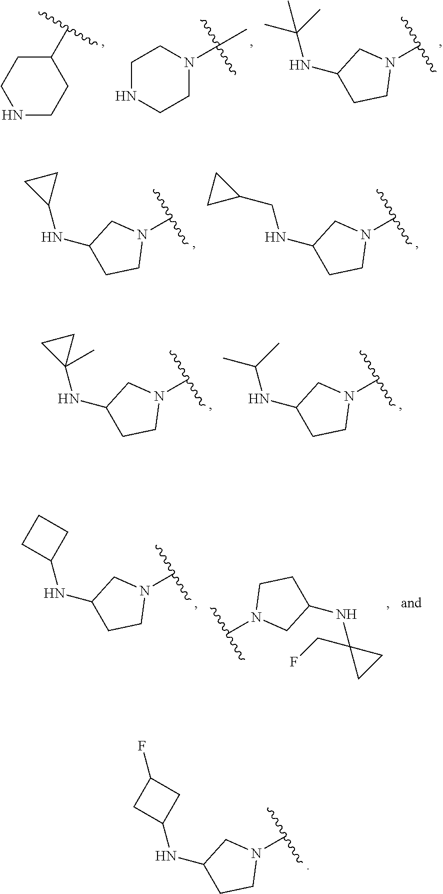

38. The compound of any one of the preceding claims, wherein A is selected from

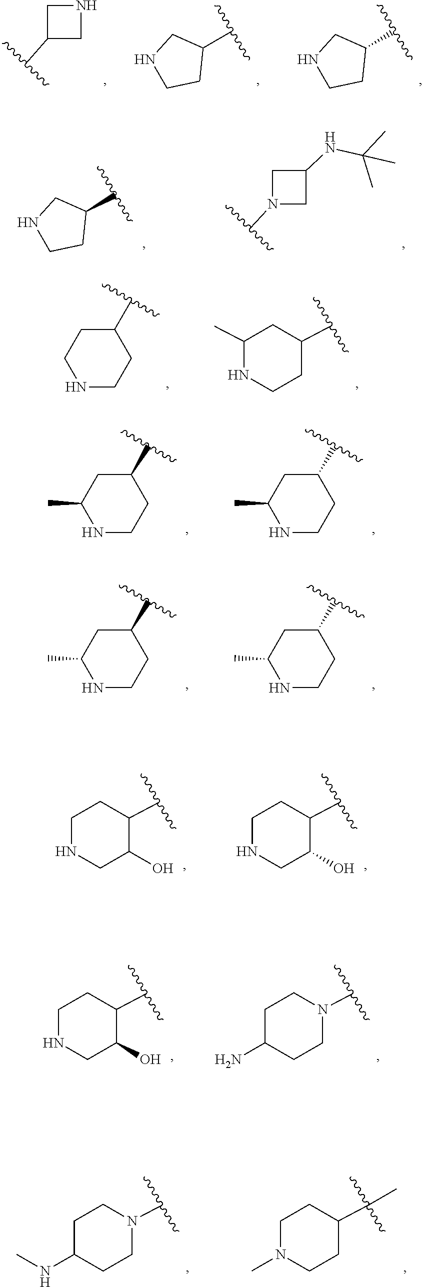

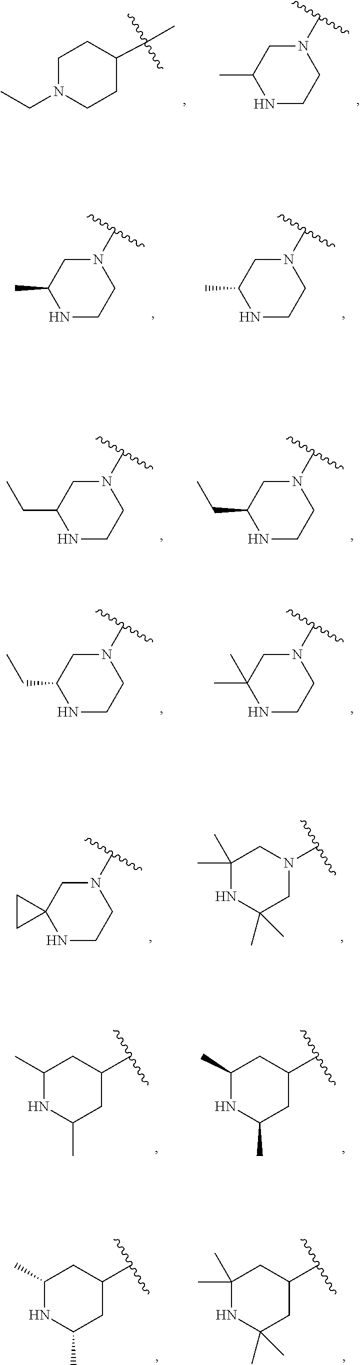

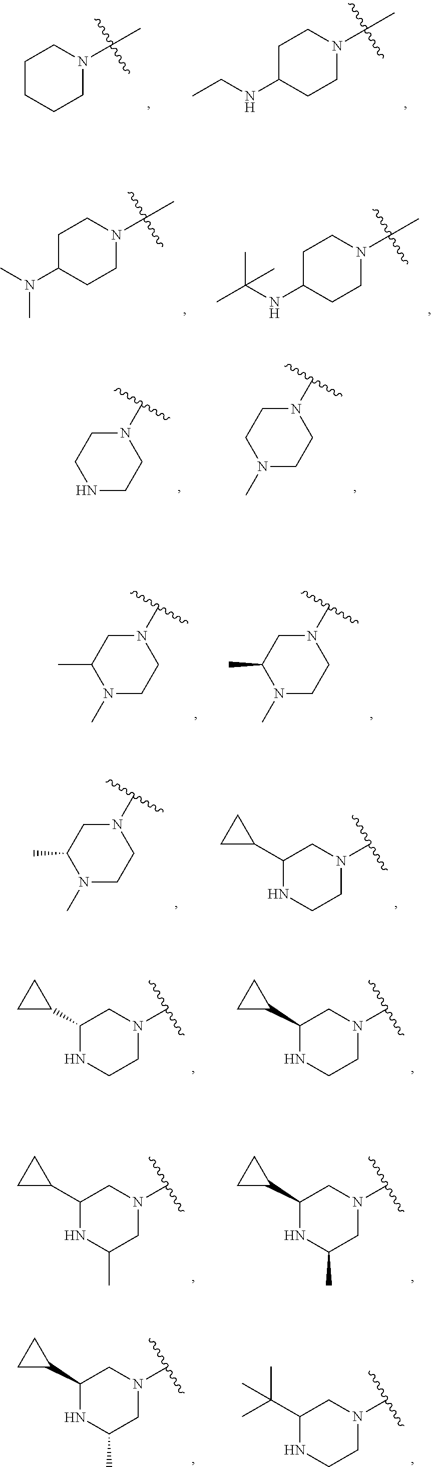

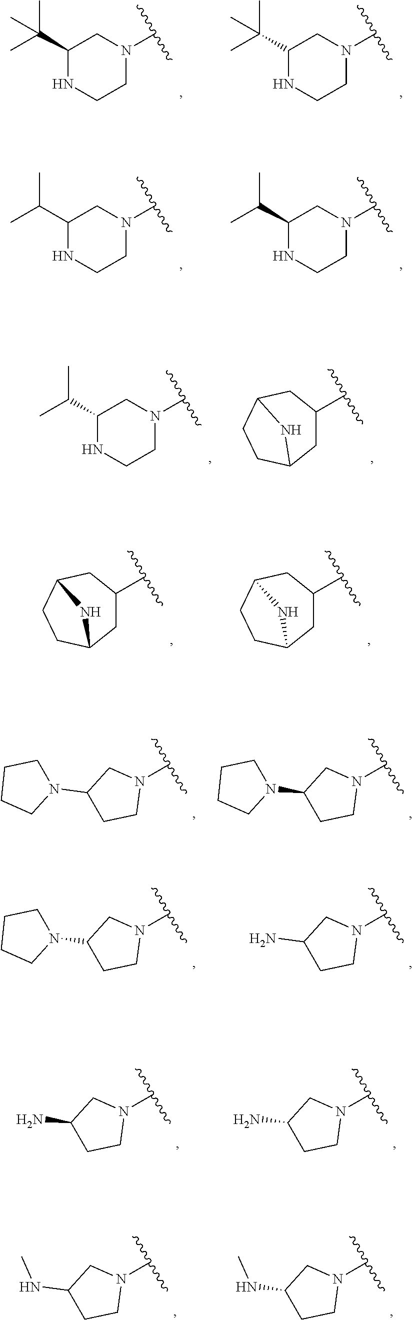

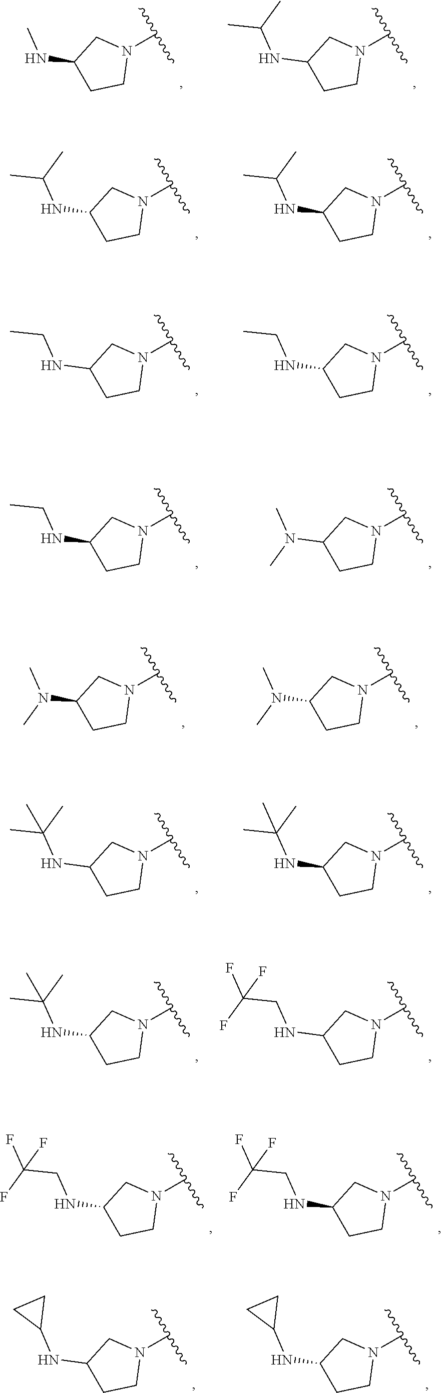

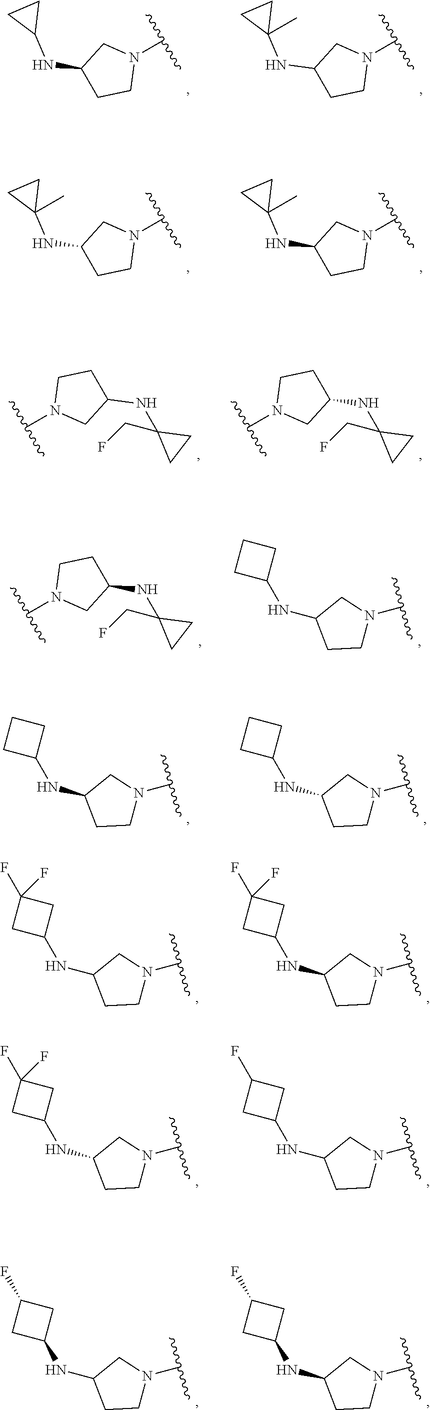

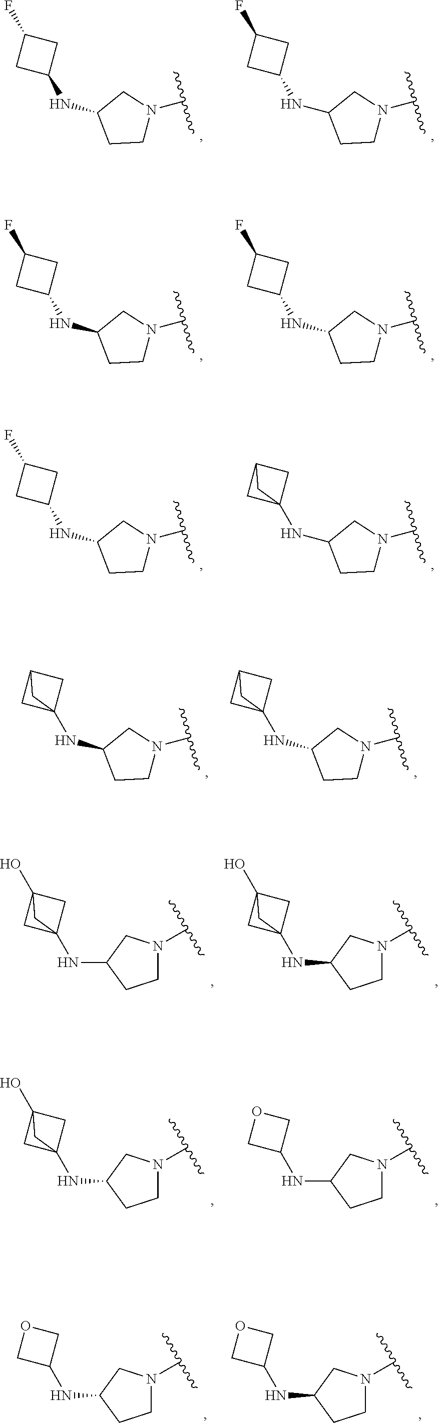

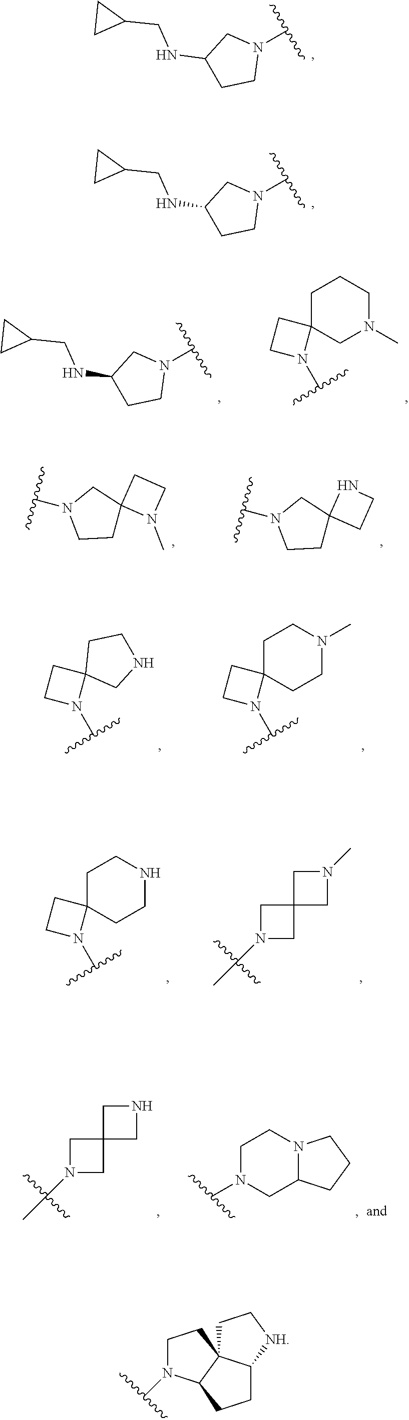

39. The compound of any one of the preceding claims, wherein B is selected

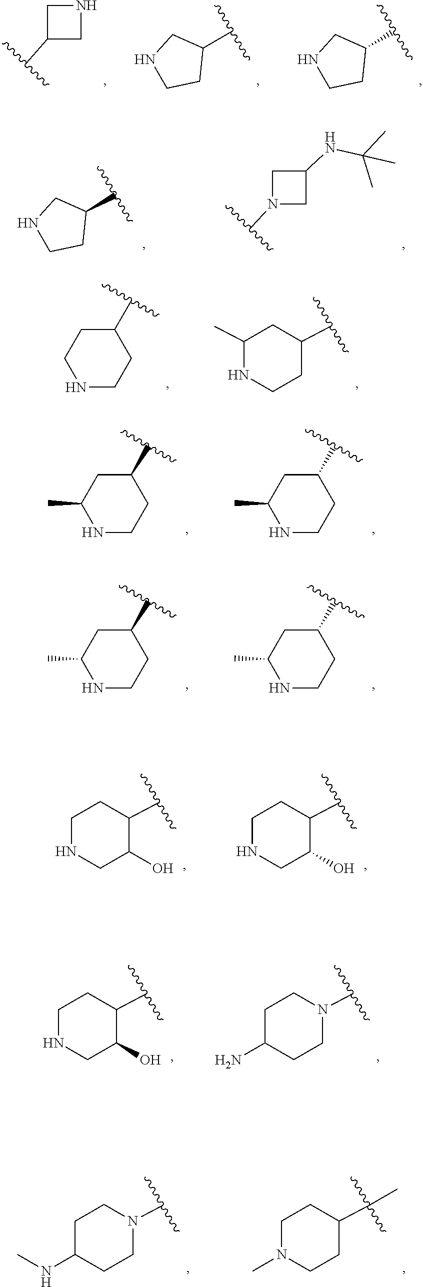

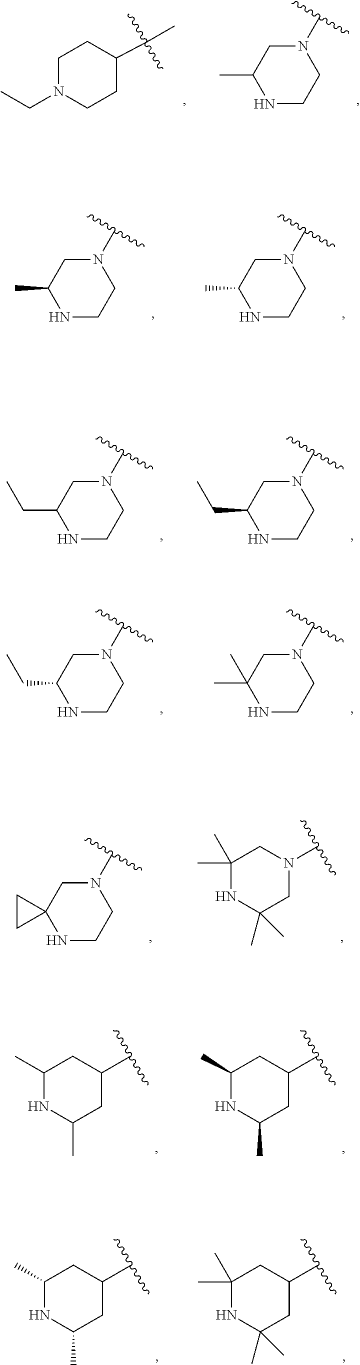

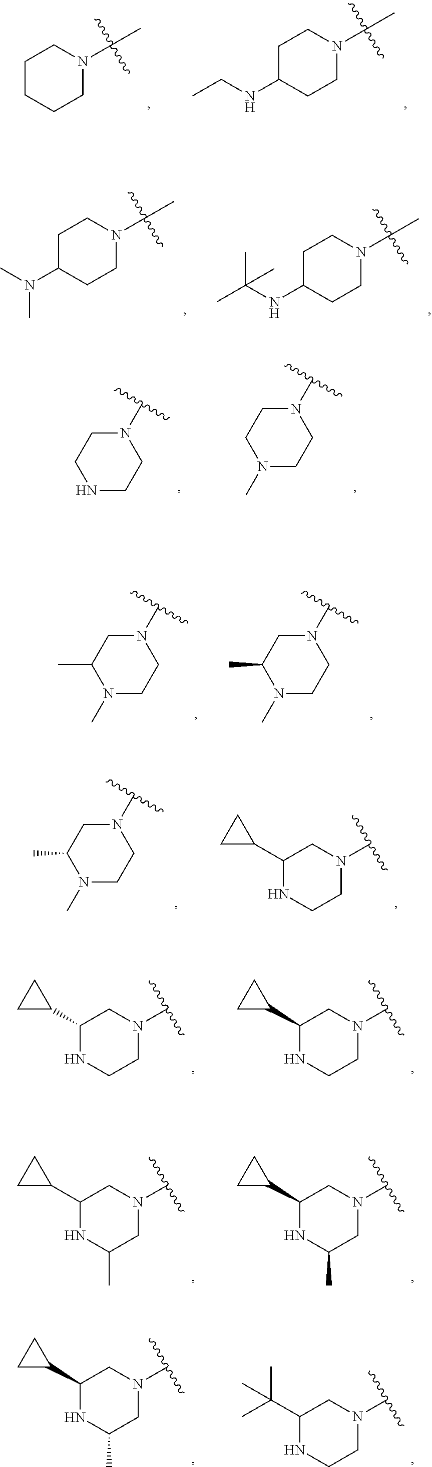

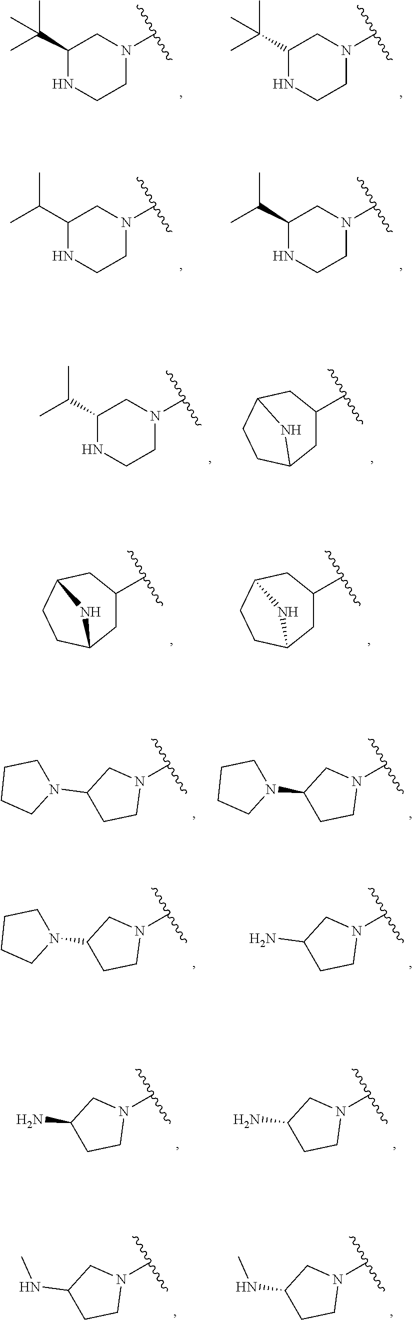

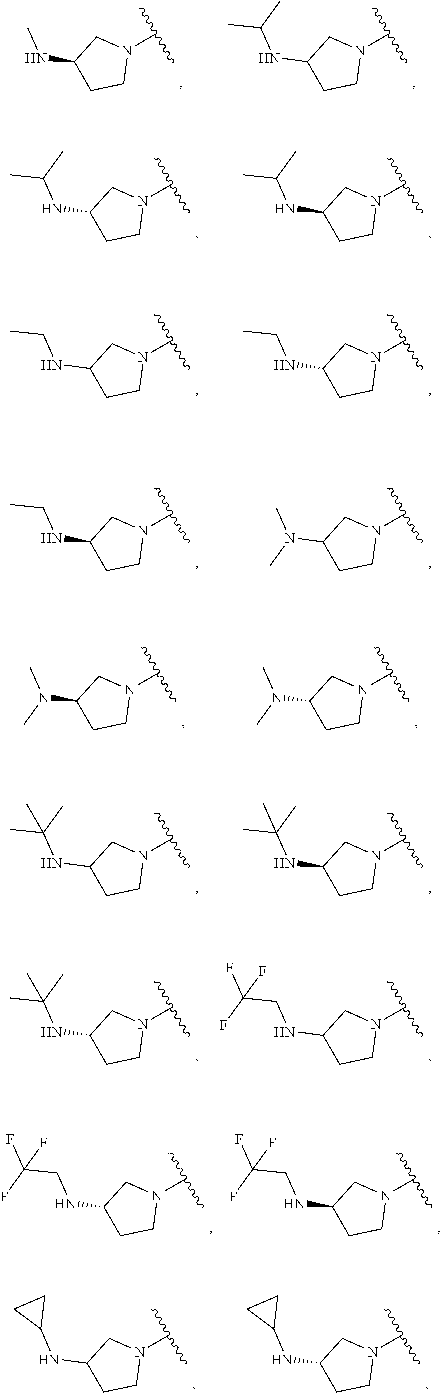

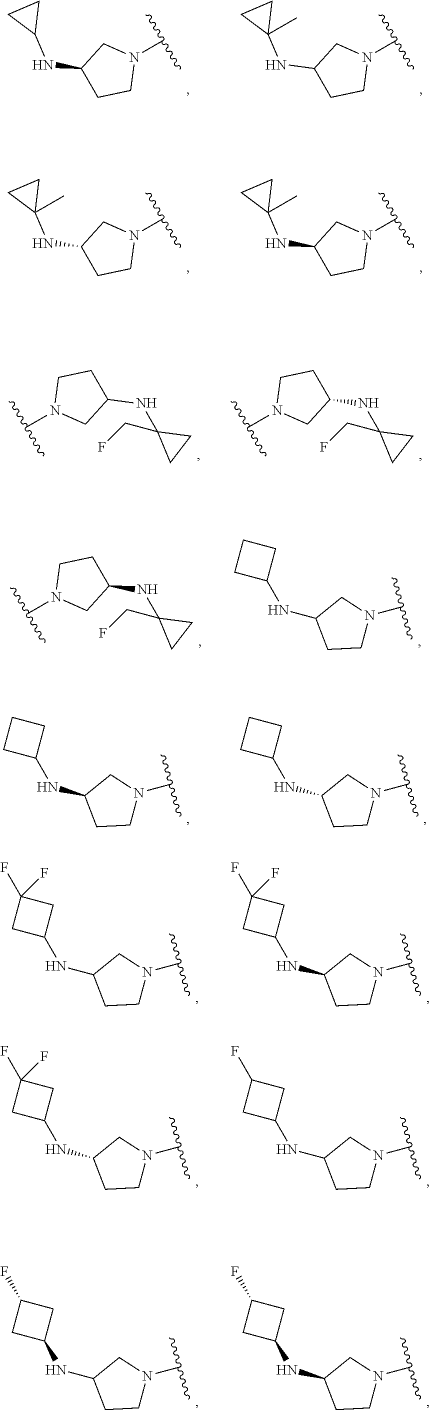

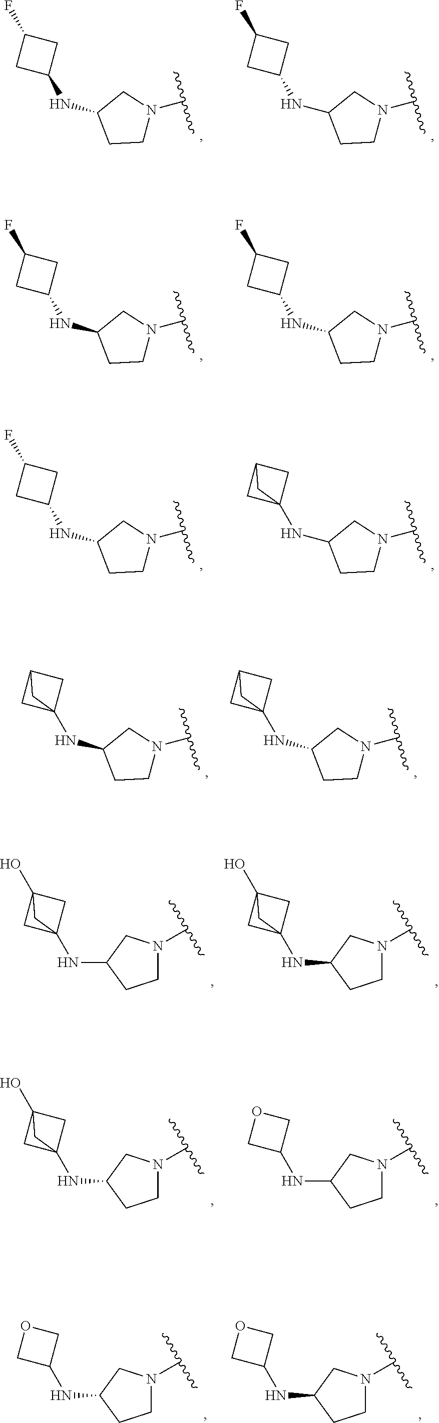

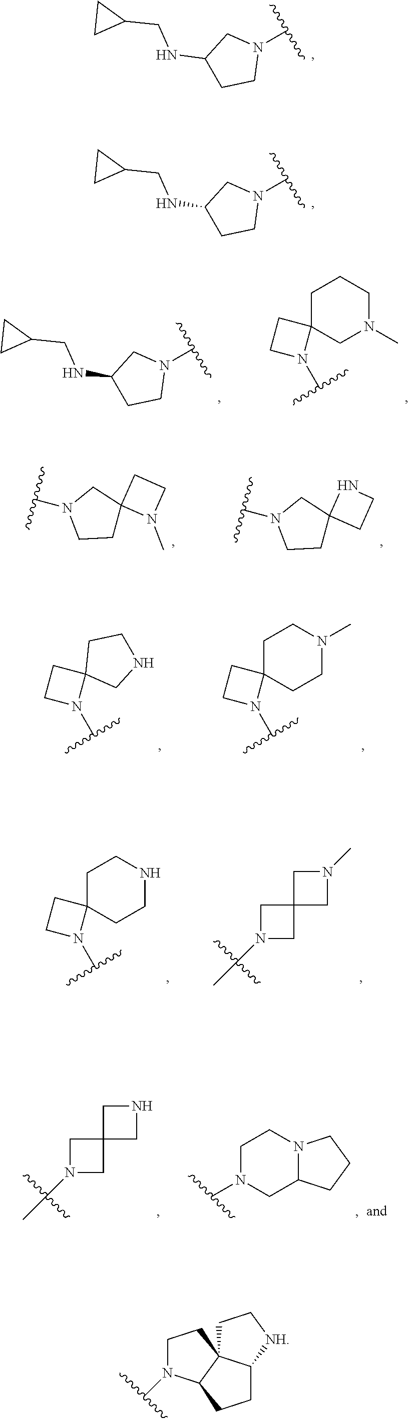

40. The compound of any one of the preceding claims, wherein both A and B are not independently heteroaryl.

41. The compound of any one of the preceding claims, wherein both of A and B are not independently heterocyclyl.

42. The compound of any one of the preceding claims, wherein one of L¹ and L² is independently absent.

43. The compound of any one of the preceding claims, wherein each of L¹ and L² is independently absent.

44. The compound of any one of claims 1-42, wherein one of L¹ and L² is independently absent and the other of L¹ and L² is independently —O— or —N(R⁴)—.

45. The compound of any one of the preceding claims, wherein X is N.

46. The compound of any one of the preceding claims, wherein Y is N.

47. The compound of any one of claims 1-44 and 46, wherein X is CH.

48. The compound of any one of claims 1-45 and 47, wherein Y is CH.

49. The compound of any one of claims 1-44, wherein one of X and Y is independently N.

50. The compound of any one of claims 1-44, wherein each of X and Y is independently N.

51. The compound of any one of claims 1-44, wherein one of X and Y is independently CH.

52. The compound of any one of the preceding claims, wherein m is 0 or 1.

53. The compound of any one of the preceding claims, wherein n is 0 or 1.

54. The compound of any one of the preceding claims, wherein each of m and n is independently 0.

55. The compound of any one of the preceding claims, wherein one of R² and R³ is independently halo (e.g., fluoro).

56. The compound of any one of the preceding claims, wherein the compound of Formula (I) is a compound of Formula (I-c): 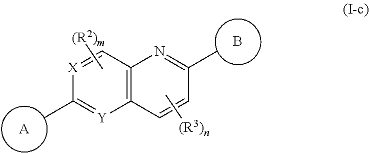
or a pharmaceutically acceptable salt, solvate, hydrate, tautomer, or stereoisomer thereof, wherein A, B, X, Y, R², R³, m, n, and subvariables thereof are as defined in claim 1.

57. The compound of claim 56, wherein A is bicyclic, nitrogen-containing heteroaryl (e.g., 6-5 bicyclic nitrogen-containing heteroaryl) optionally substituted with R¹.

58. The compound of claim 56, wherein B is bicyclic, nitrogen-containing heteroaryl (e.g., 6-5 bicyclic nitrogen-containing heteroaryl) optionally substituted with R¹.

59. The compound of claim 56, wherein A is monocyclic, nitrogen-containing heterocyclyl (e.g., 6-5 bicyclic nitrogen-containing heterocyclyl) optionally substituted with R¹.

60. The compound of claim 56, wherein B is monocyclic, nitrogen-containing heterocyclyl (e.g., 6-5 bicyclic nitrogen-containing heterocyclyl) optionally substituted with R¹.

61. The compound of any one of the preceding claims, wherein the compound of Formula (I) is a compound of Formula (I-e): 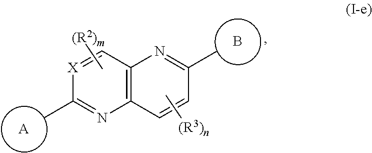
or a pharmaceutically acceptable salt, solvate, hydrate, tautomer, or stereoisomer thereof, wherein A, B, X, R², R³, m, n, and subvariables thereof are as defined in claim 1.

62. The compound of any one of the preceding claims, wherein the compound of Formula (I) is a compound of Formula (I-f): 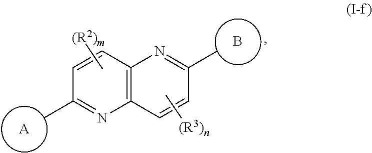
or a pharmaceutically acceptable salt, solvate, hydrate, tautomer, or stereoisomer thereof, wherein m is 0, 1 or 2; and A, B, R², R³, m, n, and subvariables thereof are as defined in claim 1.

63. The compound of any one of the preceding claims, wherein the compound of Formula (I) is a compound of Formula (I-h): 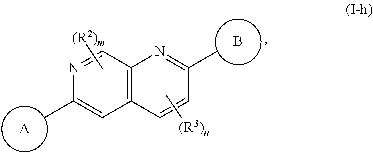
or a pharmaceutically acceptable salt, solvate, hydrate, tautomer, or stereoisomer thereof, wherein m is 0, 1 or 2; and A, B, R², R³, m, n, and subvariables thereof are as defined in claim 1.

64. The compound of any one of claims 1-58, wherein the compound of Formula (I) is a compound of Formula (I-g): 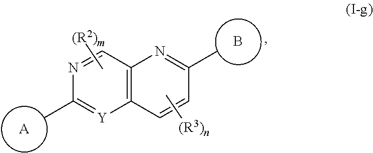
or a pharmaceutically acceptable salt, solvate, hydrate, tautomer, or stereoisomer thereof, wherein A, B, Y, R², R³, m, n, and subvariables thereof are as defined in claim 1.

65. The compound of any one of the preceding claims, wherein the compound of Formula (I) is a compound of Formula (I-h-i): 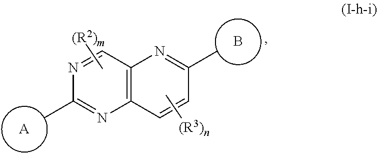
or a pharmaceutically acceptable salt, solvate, hydrate, tautomer, or stereoisomer thereof, wherein A, B, Y, R², R³, m, n, and subvariables thereof are as defined in claim 1.

66. The compound of any one of the preceding claims, wherein the compound of Formula (I) is a compound of Formula (I-k): 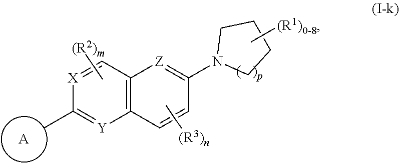
or a pharmaceutically acceptable salt, solvate, hydrate, tautomer, or stereoisomer thereof, wherein p is 0, 1, 2 or 3, and A, X, Y, Z, R², R³, m, n, and subvariables thereof are as defined in claim 1.

67. The compound of claim 66, wherein A is bicyclic, nitrogen-containing heteroaryl (e.g., 6-5 bicyclic nitrogen-containing heteroaryl) optionally substituted with R¹.

68. The compound of any one of the preceding claims, wherein the compound of Formula (I) is a compound of Formula (I-1): 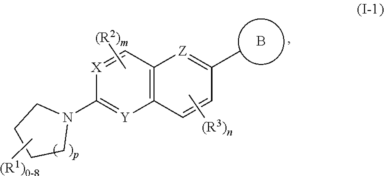
or a pharmaceutically acceptable salt, solvate, hydrate, tautomer, or stereoisomer thereof, wherein p is 0, 1, 2 or 3, and B, X, Y, Z, R², R³, m, n, and subvariables thereof are as defined in claim 1.

69. The compound of claim 68, wherein B is bicyclic, nitrogen-containing heteroaryl (e.g., 6-5 bicyclic nitrogen-containing heteroaryl) optionally substituted with R¹.

70. The compound of any one of claims 68-69, wherein R¹ is —NR^{B}R^{C} or OR^{A}.

71. The compound of any one of claims 68-70, wherein R¹ is —NR^{B}R^{C}.

72. The compound of any one of claims 68-71, wherein each of R^{B} and R^{C} is selected from hydrogen, C₁-C₆-alkyl, C₁-C₆-heteroalkyl, C₁-C₆-haloalkyl, cycloalkyl, heterocyclyl, C₁-C₆ alkylene-cycloalkyl, and C₁-C₆ alkylene-heterocyclyl, wherein each alkyl, alkylene, heteroalkyl, haloalkyl, cycloalkyl, and heterocyclyl is optionally substituted with one or more R⁹.

73. The compound of any one of the preceding claims, wherein the compound is selected from any one of the compounds shown in Table 1 or a pharmaceutically acceptable salt, solvate, hydrate, tautomer, or stereoisomer thereof.

74. A compound of Formula (II): 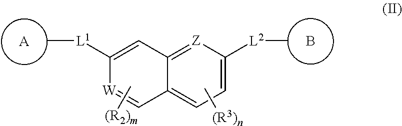
or a pharmaceutically acceptable salt, solvate, hydrate, tautomer, or stereoisomer thereof,
wherein:
A and B are each independently cycloalkyl, heterocyclyl, aryl, or heteroaryl, each of which is optionally substituted with one or more R¹;
L¹ and L² are each independently absent, C₁-C₆-alkylene, C₁-C₆-heteroalkylene, —O—, —C(O)—, —N(R⁴)—, —N(R⁴)C(O)—, —C(O)N(R⁴)—, —N(R⁴)C(O)N(R⁴)—, or C₁-C₆-alkylene-N(R⁴)C(O)N(R⁴)—, wherein each alkylene and heteroalkylene is optionally substituted with one or more R⁵;
W and Z are each N or C(R⁶), wherein at least one of W and Z is N;
each R¹ is independently hydrogen, C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, C₁-C₆ alkylene-aryl, C₁-C₆ alkenylene-aryl, C₁-C₆ alkylene-heteroaryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each alkyl, alkylene, alkenyl, alkynyl, heteroalkyl, haloalkyl, cycloalkyl, heterocyclyl, aryl, and heteroaryl is optionally substituted with one or more R⁷; or
two R¹ groups, together with the atoms to which they are attached, form a 3-7-membered cycloalkyl, heterocyclyl, aryl, or heteroaryl, wherein each cycloalkyl, heterocyclyl, aryl, and heteroaryl is optionally substituted with one or more R⁷;
each R² and R³ is independently C₁-C₆-alkyl, C₂-C₆-alkenyl, C₂-C₆-alkynyl, C₁-C₆-heteroalkyl, C₁-C₆-haloalkyl, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D};
each R⁴ is independently hydrogen, C₁-C₆-alkyl, or C₁-C₆-haloalkyl;
each R⁵ is C₁-C₆-alkyl, C₂-C₆-alkenyl, C₂-C₆-alkynyl, C₁-C₆-heteroalkyl, C₁-C₆-haloalkyl, halo, cyano, oxo, —OR^{A}, or —NR^{B}R^{C};
each R⁶ is independently hydrogen, halo, C₁-C₆-alkyl, C₁-C₆-haloalkyl, or —OR^{A};
each R⁷ is independently C₁-C₆-alkyl, C₂-C₆-alkenyl, C₂-C₆-alkynyl, C₁-C₆-heteroalkyl, C₁-C₆-haloalkyl, cycloalkyl, heterocyclyl, aryl, heteroaryl, halo, cyano, oxo, —OR^{A}, —NR^{B}R^{C}, —NR^{B}C(O)R^{D}, —NO₂, —C(O)NR^{B}R^{C}, —C(O)R^{D}, —C(O)OR^{D}, or —S(O)ₓR^{D}, wherein each of alkyl, alkenyl, alkynyl, heteroalkyl, haloalkyl, cycloalkyl, heterocyclyl, aryl, and heteroaryl is optionally substituted with one or more R⁸;
each R^{A} is independently hydrogen, C₁-C₆ alkyl, C₁-C₆ haloalkyl, aryl, heteroaryl, C₁-C₆ alkylene-aryl, C₁-C₆ alkylene-heteroaryl, —C(O)R^{D}, or —S(O)ₓR^{D};
each R^{B} and R^{C} is independently hydrogen, C₁-C₆ alkyl, C₁-C₆ heteroalkyl, cycloalkyl, heterocyclyl, or —OR^{A}; or
R^{B} and R^{C} together with the atom to which they are attached form a 3-7-membered heterocyclyl ring optionally substituted with one or more R⁹;
each R^{D} is independently hydrogen, C₁-C₆ alkyl, C₂-C₆ alkenyl, C₂-C₆ alkynyl, C₁-C₆ heteroalkyl, C₁-C₆ haloalkyl, cycloalkyl, heterocyclyl, aryl, heteroaryl, C₁-C₆ alkylene-aryl, or C₁-C₆ alkylene-heteroaryl;
each R⁸ is independently C₁-C₆ alkyl, C₁-C₆ heteroalkyl, C₁-C₆ haloalkyl, cycloalkyl, heterocyclyl, aryl, heteroaryl, halo, cyano, oxo, or —OR^{A};
each R⁹ is C₁-C₆-alkyl, halo, cyano, oxo, or —OR^{A1};
each R^{A1} is hydrogen or C₁-C₆-alkyl;
m and n are each independently 0, 1, or 2; and
x is 0, 1, or 2.

75. The compound of claim 74, wherein one of A and B is independently a monocyclic heteroaryl or bicyclic heteroaryl, each of which is optionally substituted with one or more R¹.

76. The compound of any one of claims 74-75, wherein one of A and B is independently a bicyclic heteroaryl optionally substituted with one or more R¹.

77. The compound of any one of claims 74-76, wherein one of A and B is independently selected from 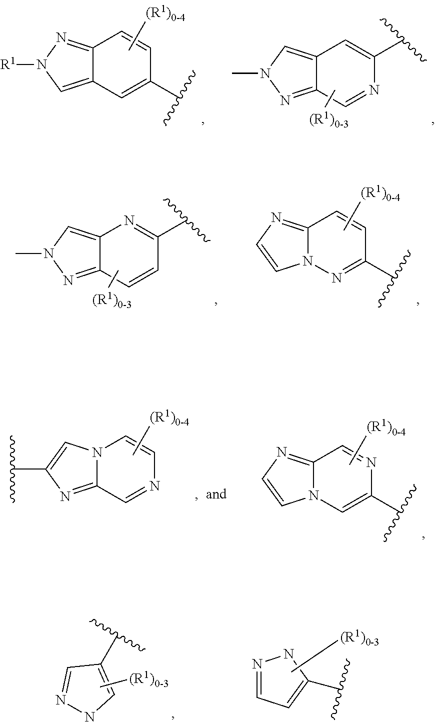
wherein R¹ is as described in claim 74.

78. The compound of any one of claims 74-77, wherein one of A and B is independently is

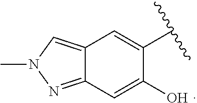 [SMILES: *c1cc2cn(C)nc2cc1O]

79. The compound of any one of claims 74-78, wherein one of A and B is independently a nitrogen-containing heterocyclyl optionally substituted with one or more R¹.

80. The compound of any one of claims 74-79, wherein one of A and B is independently a 4-8 membered heterocyclyl optionally substituted with one or more R¹.

81. The compound of any one of claims 74-80, wherein one of A and B is independently selected from 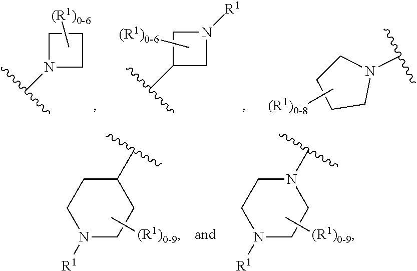
wherein R¹ is as described in claim 74.

82. The compound of any one of claims 74-81, wherein one of L¹ and L² is independently absent.

83. The compound of any one of claims 74-82, wherein each of L¹ and L² is independently absent.

84. The compound of any one of claims 74-81, wherein each of L¹ and L² is absent, —N(R⁴)C(O)N(R⁴)—, or C₁-C₆-alkylene-N(R⁴)C(O)N(R⁴)—.

85. The compound of any one of claims 74-84, wherein W is N.

86. The compound of any one of claims 74-84, wherein Z is N.

87. The compound of any one of claims 74-86, wherein each of W and Z is independently N.

88. The compound of any one of claims 74-87, wherein the compound of Formula (II) is a compound of Formula (II-a): 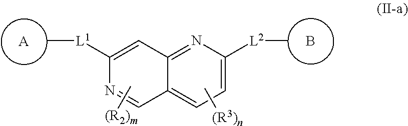
or a pharmaceutically acceptable salt, solvate, hydrate, tautomer, or stereoisomer thereof, wherein A, B, L¹, L², R², R³, m, n, and subvariables thereof are as defined in claim 69.

89. The compound of any one of claims 74-87, wherein the compound of Formula (II) is a compound of Formula (II-b): 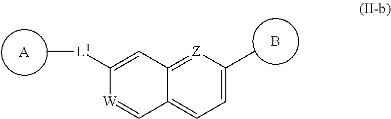
or a pharmaceutically acceptable salt, solvate, hydrate, tautomer, or stereoisomer thereof, wherein A, B, L¹, W, Z, and subvariables thereof are as defined in claim 74.

90. The compound of any one of claims 74-89, wherein the compound of Formula (II) is a compound of Formula (II-c): 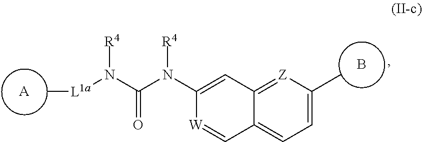
or a pharmaceutically acceptable salt, solvate, hydrate, tautomer, or stereoisomer thereof, wherein Lia is absent or C₁-C₆-alkylene, and A, B, L^{1a}, W, Z, R⁴, and subvariables thereof are as defined in claim 74.

91. The compound of any one of claims 74-90, wherein the compound is selected from any one of the compounds shown in Table 2 or a pharmaceutically acceptable salt, solvate, hydrate, tautomer, or stereoisomer thereof.

92. A pharmaceutical composition comprising a compound of any one of claims 1-91 and a pharmaceutically acceptable excipient.

93. The compound of any one of claims 1-91 or the pharmaceutical composition of claim 92, wherein the compound alters a target nucleic acid (e.g., an RNA, e.g., a pre-mRNA).

94. The compound of any one of claims 1-91 or the pharmaceutical composition of claim 92, wherein the compound binds to a target nucleic acid (e.g., an RNA, e.g., a pre-mRNA).

95. The compound of any one of claims 1-91 or the pharmaceutical composition of claim 92, wherein the compound stabilizes a target nucleic acid (e.g., an RNA, e.g., a pre-mRNA).

96. The compound of any one of claims 1-91 or the pharmaceutical composition of claim 92, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9% 10%, 15%, 20%, 25%, 30%, 35%, 40%, 45%, 50%, 55%, 60%, 65%, 70%, 75%, 80%, 85%, 90%, 95%, or more, e.g., as determined by qPCR.

97. The compound of any one of claims 1-91 or the pharmaceutical composition of claim 92, wherein the compound decreases splicing at splice site on a target nucleic acid (e.g., an RNA, e.g., a pre-mRNA), by about 0.5%, 1%, 2%, 3% 4%, 5%, 6%, 7%, 8%, 9% 10%, 15%, 20%, 25%, 30%, 35%, 40%, 45%, 50%, 55%, 60%, 65%, 70%, 75%, 80%, 85%, 90%, 95%, or more, e.g., as determined by qPCR %.

98. A method of modulating splicing of a nucleic acid (e.g., DNA, RNA, e.g., a pre-mRNA) comprising contacting the nucleic acid with a compound of Formula (I) or (II) as described in any one of claims 1-91 or a pharmaceutical composition of claim 92.

99. The method of claim 98, wherein the compound increases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%, 40%, 45%, 50%, 55%, 60%, 65%, 70%, 75%, 80%, 85%, 90%, 95%, or more, e.g., as determined by qPCR.

100. The method of claim 98, wherein the compound decreases splicing at splice site on a target nucleic acid (e.g., an RNA, e.g., a pre-mRNA), by about 0.5%, 1%, 2%, 3%, 4%, 5%, 6%, 7%, 8%, 9%, 10%, 15%, 20%, 25%, 30%, 35%0, 40%, 45%, 50%, 55%, 60%, 65%, 70%, 75%, 80%, 85%, 90%, 95%, or more, e.g., as determined by qPCR.

101. A method of forming a complex comprising a component of a spliceosome (e.g., a major spliceosome component or a minor spliceosome component), a nucleic acid (e.g., a DNA, RNA, e.g., a pre-mRNA), and a compound of Formula (I) or Formula (II):
comprising contacting the nucleic acid (e.g., a DNA, RNA, e.g., a pre-mRNA) with a compound of Formula (I) or (II), according to any one of claims 1-91 or the pharmaceutical composition of claim 92.

102. The method of claim 101, wherein the component of a spliceosome is recruited to the nucleic acid in the presence of the compound of Formula (I) or (II).

103. A method of altering the conformation of a nucleic acid (e.g., a DNA, RNA, e.g., a pre-mRNA) comprising contacting the nucleic acid with a compound of Formula (I) or (II), according to any one of claims 1-91 or the pharmaceutical composition of claim 92.

104. The method of claim 103, wherein the altering comprises forming a bulge in the nucleic acid.

105. The method of claim 103, wherein the altering comprises stabilizing a bulge in the nucleic acid.

106. The method of claim 103, wherein the altering comprises reducing a bulge in the nucleic acid.

107. The method of any one of claims 103-106, wherein the nucleic acid comprises a splice site.

108. A composition for use in treating a disease or disorder in a subject comprising administering to the subject a compound of Formula (I) or (II), according to any one of claims 1-91 or the pharmaceutical composition of claim 92.

109. The composition for use of claim 103, wherein the disease or disorder comprises a proliferative disease (e.g., cancer, a benign neoplasm, or angiogenesis).

110. The composition for use of claim 103, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

111. The composition for use of claim 103, wherein the disease or disorder comprises neurological disease or disorder.

112. The composition for use of claim 103, wherein the disease or disorder comprises Huntington's disease.

113. A method for treating a disease or disorder in a subject comprising administering to the subject a compound of Formula (I) or (II), according to any one of claims 1-91 or the pharmaceutical composition of claim 92.

114. The method of claim 113, wherein the disease or disorder comprises a proliferative disease (e.g., cancer, a benign neoplasm, or angiogenesis).

115. The method of claim 114, wherein the disease or disorder comprises a neurological disease or disorder, autoimmune disease or disorder, immunodeficiency disease or disorder, lysosomal storage disease or disorder, cardiovascular disease or disorder, metabolic disease or disorder, respiratory disease or disorder, renal disease or disorder, or infectious disease.

116. The method of claim 115, wherein the disease or disorder comprises neurological disease or disorder.

117. The method of claim 115, wherein the disease or disorder comprises Huntington's disease.
